(19) United States
(12) Patent Application Publication
(10) Pub. No.: US 2026/0015350 A1
(43) Pub. Date: Jan. 15, 2026

(54) AZAQUINAZOLINE PAN-KRAS INHIBITORS

(71) Applicant: Mirati Therapeutics, Inc., Princeton, NJ (US)

(72) Inventors: Xiaolun WANG, San Diego, CA (US); Svitlana KULYK, San Diego, CA (US); John David LAWSON, Carlsbad, CA (US); Matthew Arnold MARX, San Diego, CA (US); Christopher Ronald SMITH, San Diego, CA (US)

(21) Appl. No.: 18/875,610

(22) PCT Filed: Jun. 13, 2023
(86) PCT No.: PCT/US2023/025214

§ 371 Date: Dec. 16, 2024

Related U.S. Application Data
(60) Provisional application No. 63/352,496, filed on Jun. 15, 2022.
(60) Provisional application No. 63/438,715, filed on Jan. 12, 2023.
(60) Provisional application No. 63/459,559, filed on Apr. 14, 2023.

(51) Int. Cl. C07D 471/04; A61K 31/519; A61K 31/5355; A61K 31/537; A61K 31/5377; A61K 31/5383; A61K 31/541; A61K 31/547; A61K 31/549; A61K 31/55; A61K 31/551; A61K 31/553; A61K 31/554; A61K 31/675; C07D 519/00; C07F 9/6561

(52) U.S. Cl. CPC C07D 471/04; A61K 31/519; A61K 31/5355; A61K 31/537; A61K 31/5377; A61K 31/5383; A61K 31/541; A61K 31/547; A61K 31/549; A61K 31/55; A61K 31/551; A61K 31/553; A61K 31/554; A61K 31/675; C07D 519/00; C07F 9/6561

(57) ABSTRACT

The present invention relates to compounds that inhibit at least one of KRas G12A, KRas G12C, KRas G12D, KRas G12R, KRas G12S, KRas G12V, KRas G13D and KRas Q61H, pharmaceutical compositions comprising the compounds and methods of use therefor.

### FIELD OF THE INVENTION

The present invention relates to compounds that inhibit multiple mutated forms of KRas, i.e., pan-KRas inhibitors. In particular, the present invention relates to pan-KRas compounds, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas. KRas mutations at codons 12, 13, 61 and other positions of the KRas primary amino acid sequence are present in 88% of all pancreatic adenocarcinoma patients, 50% of all colon/rectal adenocarcinoma patients, and 32% lung adenocarcinoma patients (e.g., see Prior et all., (2020) Cancer Res 80:2969-74). A recent publication also suggested wild type Kras inhibition could be a viable therapeutic strategy to treat KRas^{WT} dependent cancers (e.g., see Bery et al., (2020) Nat. Commun. 11:3233).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8): 1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51 (25): 6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants-.

Thus, there is a need to develop new pan-KRas inhibitors that demonstrate sufficient efficacy for treating KRas-mediated cancers.

### SUMMARY OF THE INVENTION

In one aspect of the invention, compounds are provided that inhibit KRas activity. In certain embodiments, the compounds are represented by Formula (I):

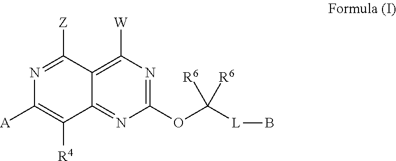

or a pharmaceutically acceptable salt thereof, wherein:

W is:

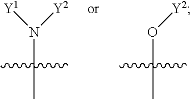

A is aryl or heteroaryl, wherein the aryl or the heteroaryl is optionally substituted with 1-4 R¹;
B is:

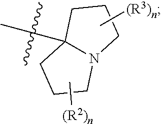

Y¹ is hydrogen, L-hydroxy optionally substituted with 1-4 R⁸, L-alkoxy optionally substituted with 1-4 R⁸, halogen, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle substituted with 1-2 oxo (═O) or oxo-containing substituent, and optionally further substituted with 1-2 R⁸;
Y² is hydrogen or C1-C4 alkyl;
or Y¹ and Y² join to form:

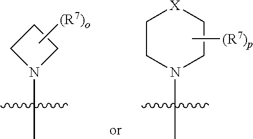

where X is selected from: a bond, —S—, —O—, —N<bound to a fused ring, —CH₂—, —CH₂—N—, —CH₂—N—CH₂—, —CH₂—CH₂—CH₂—, —CH₂—CH₂—, —O—CH₂— and —S—CH₂—;
or Y² and Z join to form V, where Vis:

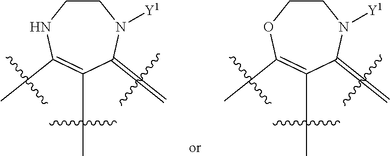

optionally substituted with 1-4 R⁸;
Z is hydrogen or joins with Y²;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, cyano, C1-C3 alkyl, C1-C3 cyanoalkyl, C1-C3 hydroxyalkyl, HC(═O)—, -L-OC(O)N(R⁵)₂, —CO₂R⁵, —CON(R⁵)₂, ═CH₂, ═CHR¹¹ or ═C(R¹¹)₂;
each R³ is independently hydrogen, hydroxy, halogen, cyano, C1-C3 alkyl, C1-C3 cyanoalkyl, C1-C3 hydroxyalkyl, HC(═O)—, -L-OC(O)N(R⁵)₂, —CO₂R⁵, —CO₂N(R⁵)₂, ═CH₂, —CHR¹¹ or ═C(R¹¹)₂;
wherein if V is not present, at least one of R² and R³ are ═CH₂, ═CHR¹¹ or ═C(R¹¹)₂;
each R⁴ is independently hydrogen, halogen or C1-C3 alkyl;
each R⁵ is independently hydrogen or C1-C3 alkyl, or two R⁵ join to form cycloalkyl or heterocycle;
each R⁶ is independently hydrogen, hydroxy, C1-C4 hydroxyalkyl or heteroaryl,
or two R⁶ join to form C3-C6 cycloalkyl or heterocycle;
each R⁷ is independently hydrogen, C1-C3 alkyl, hydroxy, halogen, C1-C3 haloalkyl, -L-NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)N(R¹⁰)₂, —NHC(O)H, —CN, aryl, —(CH₂)₁₋₂S(O)₂N(R¹⁰)₂, —NH—S(O)₂N(R¹⁰)₂, —O—S(O)₂N(R¹⁰)₂, S(O)₂R¹⁰, or heteroaryl or heterocycle optionally independently substituted with 1-2 substituents independently selected from C1-C3 alkyl, —CN and C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a 1-2 carbon bridge;
each R⁸ is independently C1-C3 alkyl, hydroxy, halogen, —N(R¹⁰)₂, —N(R¹⁰)C(O)R¹⁰, oxo (═O), —O—(C1-C3 alkyl), —(C1-C3 alkyl)-OH, —C(O)OH, —C(O)O(C1-C3 alkyl), —C(O)N(R¹⁰)₂, heteroaryl or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
each R¹⁰ is independently hydrogen, halogen, C1-C3 alkyl, or two R¹⁰ join to form cycloalkyl or heterocycle optionally substituted with 1-2 C1-C3 alkyl;
each R¹¹ is independently halogen;
each L is independently a bond, —C1-C4 alkyl-, —C1-C4 alkyl-NH—, —NH—, —N(C1-C3 alkyl)- or cyclopropyl-CH₂—;
each n is 0-3;
is 1-6; and
p is 1-8.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting the activity of cells containing wild type KRas or one or more KRas mutations, for instance the KRas mutations G12A, G12C, G12D, G12R, G12S, G12V, G13D and/or Q61H, in a in a cell, comprising contacting the cell with a compound of Formula (I), or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a KRas wild type, KRas G12A, G12C, G12D, G12R, G12S, G12V, G13D and/or Q61H-associated disease or disorder in a patient in need of such treatment, the method comprising administering to the patient a therapeutically effective amount of a compound of Formula (I), or a pharmaceutically acceptable salt thereof, or a pharmaceutical composition thereof as defined herein.

Also provided herein is a compound of Formula (I), or a pharmaceutically acceptable salt thereof, or a pharmaceutical composition thereof as defined herein for use in therapy.

Also provided herein is a compound of Formula (I), or a pharmaceutically acceptable salt thereof or a pharmaceutical composition thereof as defined herein for use in the treatment of cancer.

Also provided herein is a compound of Formula (I), or a pharmaceutically acceptable salt thereof for use in the inhibition of KRas wild type or multiple types of KRas mutations, for instance KRas G12A, G12C, G12D, G12R, G12S, G12V, G13D and/or Q61H mutations.

Also provided herein is a compound of Formula (I), or a pharmaceutically acceptable salt thereof or a pharmaceutical composition thereof as defined herein, for use in the treatment of a KRas wild type associated disease or disorder or a KRas mutation G12A, G12C, G12D, G12R, G12S, G12V, G13D and/or Q61H-associated disease or disorder.

Also provided herein is the use of a compound of Formula (I), or a pharmaceutically acceptable salt thereof, as defined herein in the manufacture of a medicament for the treatment of cancer.

Also provided herein is a use of a compound of Formula (I), or a pharmaceutically acceptable salt thereof, as defined herein in the manufacture of a medicament for the inhibition of activity of the wild type form of KRas or mutated forms of KRas, including the mutations: G12A, G12C, G12D, G12R, G12S, G12V, G13D and/or Q61H.

Also provided herein is the use of a compound of Formula (I), or a pharmaceutically acceptable salt thereof, as defined herein, in the manufacture of a medicament for the treatment of a KRas wild type associated disease or disorder or a KRas G12A, G12C, G12D, G12R, G12S, G12V, G13D and/or Q61H-associated disease or disorder.

Also provided herein is a method for treating cancer in a patient in need thereof, the method comprising (a) determining that the cancer is associated with KRas wild type or a KRas G12A, G12C, G12D, G12R, G12S, G12V, G13D and/or Q61H mutation (i.e., a KRas G12A, G12C, G12D, G12R, G12S, G12V, G13D and/or Q61H-associated cancer); and (b) administering to the patient a therapeutically effective amount of a compound of Formula (I), or a pharmaceutically acceptable salt thereof, or a pharmaceutical composition thereof.

One potential utility of the herein-described pan-KRas inhibitors, including pan-KRas inhibitors such as (R)-1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol (Example 5 herein), is for the treatment of cancers that develop resistance following long-term treatment with KRas G12C inhibitors. Thus, embodiments of the invention include those wherein a patient suffering from cancer is treated with a herein-described pan-KRas inhibitor such as Example 5 after treatment with a G12C inhibitor becomes ineffective or less effective due to the emergence of resistance-imparting mutations.

Treatment of KRas G12C mutant cancers with covalent KRas G12C inhibitors such as adagrasib (MRTX849) or sotorasib (AMG510) may result in the incorporation of additional mutations that confer resistance to adagrasib. These mutations could confer resistance through numerous mechanisms.

Mutations that change the mutant cysteine at codon 12 to another amino acid would render the current covalent KRas G12C inhibitors ineffective since current inhibitors make a covalent bond with the mutant cysteine amino acid side chain. Likewise, in patients that have one wild type KRas allele in addition to the KRas G12C-mutant allele, mutations in the wild type codon 12 glycine to another codon would allow bypass signaling in these tumors through the novel mutant protein. The repertoire of codon 12 mutations that can occur with a single nucleotide substitution in the wild type gene (glycine codon) includes mutations commonly observed in cancer such as G12S, G12V, G12R, G12C. The repertoire of codon 12 mutations that can occur with single nucleotide base substitutions of the cysteine codon 12 include mutations not frequently observed in cancer, G12Y, G12F and G12W, in addition to G12S and G12R.

Second-site mutations may also occur in another location in the KRas G12C mutant gene that confers resistance to KRas G12C inhibitor treatment. These mutations may confer resistance through different mechanisms. RAS proteins are small GTPases that normally cycle between an active, GTP-bound state and an inactive, GDP-bound state. RAS proteins are loaded with GTP through guanine nucleotide exchange factors (GEFs; e.g., SOS1) which are activated by upstream receptor tyrosine kinases, triggering subsequent interaction with effector proteins that activate RAS-dependent signaling. RAS proteins hydrolyze GTP to GDP through their intrinsic GTPase activity which is dramatically enhanced by GTPase-activating proteins (GAPs). Mutations at codons 12 and 13 in RAS proteins impair GAP-stimulated GTP hydrolysis leaving RAS predominantly in the GTP-bound, active state. Covalent KRas G12C inhibitors in current clinical development only bind GDP-bound KRas G12C. Mutations such as Q61 codon mutations, which may or may not occur on the same allele as the G12C mutation, reduce the intrinsic GTPase activity of KRas and may represent a mechanism of resistance to KRas G12C inhibitor treatment by shifting KRas into the GTP-loaded state where it is not susceptible to covalent inhibition. Co-mutations such as R68, H95 and Y96 may be present along with the KRas G12C mutation and may diminish the binding affinity of KRas G12C inhibitors to the Switch II binding pocket.

The herein-described pan-KRas inhibitors may demonstrate activity against common as well as uncommon codon 12 mutations or mutations that occur in the KRas protein that diminish binding of KRas G12C inhibitors to the KRas protein.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

### DETAILED DESCRIPTION OF THE INVENTION

The present invention relates to inhibitors of KRas wild type and/or multiple mutated forms of KRas, for instance KRas G12A, G12C, G12D, G12R, G12S, G12V, G13D and/or Q61H mutations. In particular, the present invention relates to compounds that inhibit the activity of KRas wild type and/or KRas mutations such as G12A, G12C, G12D, G12R, G12S, G12V, G13D and/or Q61H, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “wild type KRas” refers to a non-mutant form of a mammalian KRas protein. The assignment of amino acid codon and residue positions for human KRas is based on the amino acid sequence identified by UniProtKB/Swiss-Prot P01116: Variantp.Gly12Asp. As used herein, a “wild type KRas inhibitor” refers to compounds of the present invention that are represented by Formula (I), as described herein. These compounds are capable of negatively modulating or inhibiting all or a portion of the enzymatic activity of wild type KRas G12A. A “wild type KRas-associated disease or disorder” as used herein refers to diseases or disorders associated with or mediated by or having wild type KRas. A non-limiting example of a wild type KRas-associated disease or disorder is a wild type KRas-associated cancer.

As used herein, “KRas G12A” refers to a mutant form of a mammalian KRas protein that contains an amino acid substitution of an alanine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12A inhibitor” refers to compounds of the present invention that are represented by Formula (I), as described herein. These compounds are capable of negatively modulating or inhibiting all or a portion of the enzymatic activity of KRas G12A. A “KRas G12A-associated disease or disorder” as used herein refers to diseases or disorders associated with or mediated by or having a KRas G12A mutation. A non-limiting example of a KRas G12A-associated disease or disorder is a KRas G12A-associated cancer.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12C inhibitor” refers to compounds of the present invention that are represented by Formula (I), as described herein. These compounds are capable of negatively modulating or inhibiting all or a portion of the enzymatic activity of KRas G12C. A “KRas G12C-associated disease or disorder” as used herein refers to diseases or disorders associated with or mediated by or having a KRas G12C mutation. A non-limiting example of a KRas G12C-associated disease or disorder is a KRas G12CD-associated cancer.

As used herein, “KRas G12D” refers to a mutant form of a mammalian KRas protein that contains an amino acid substitution of an aspartic acid for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12D inhibitor” refers to compounds of the present invention that are represented by Formula (I), as described herein. These compounds are capable of negatively modulating or inhibiting all or a portion of the enzymatic activity of KRas G12D. A “KRas G12D-associated disease or disorder” as used herein refers to diseases or disorders associated with or mediated by or having a KRas G12D mutation. A non-limiting example of a KRas G12D-associated disease or disorder is a KRas G12D-associated cancer.

As used herein, “KRas G12R” refers to a mutant form of a mammalian KRas protein that contains an amino acid substitution of an arginine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12R inhibitor” refers to compounds of the present invention that are represented by Formula (I), as described herein. These compounds are capable of negatively modulating or inhibiting all or a portion of the enzymatic activity of KRas G12R. A “KRas G12R-associated disease or disorder” as used herein refers to diseases or disorders associated with or mediated by or having a KRas G12R mutation. A non-limiting example of a KRas G12R-associated disease or disorder is a KRas G12R-associated cancer.

As used herein, “KRas G12S” refers to a mutant form of a mammalian KRas protein that contains an amino acid substitution of a serine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12S inhibitor” refers to compounds of the present invention that are represented by Formula (I), as described herein. These compounds are capable of negatively modulating or inhibiting all or a portion of the enzymatic activity of KRas G12S. A “KRas G12S-associated disease or disorder” as used herein refers to diseases or disorders associated with or mediated by or having a KRas G12S mutation. A non-limiting example of a KRas G12S-associated disease or disorder is a KRas G12S-associated cancer.

As used herein, “KRas G12V” refers to a mutant form of a mammalian KRas protein that contains an amino acid substitution of a valine for a glycine at amino acid position 12. The assignment of amino acid codon and residue positions for human KRas is based on the amino acid sequence identified by UniProtKB/Swiss-Prot P01116: Variantp.Gly12Asp. As used herein, a “KRas G12V inhibitor” refers to compounds of the present invention that are represented by Formula (I), as described herein. These compounds are capable of negatively modulating or inhibiting all or a portion of the enzymatic activity of KRas G12V. A “KRas G12V-associated disease or disorder” as used herein refers to diseases or disorders associated with or mediated by or having a KRas G12V mutation. A non-limiting example of a KRas G12V-associated disease or disorder is a KRas G12V-associated cancer.

As used herein, “KRas G13D” refers to a mutant form of a mammalian KRas protein that contains an amino acid substitution of an aspartic acid for a glycine at amino acid position 13. The assignment of amino acid codon and residue positions for human KRas is based on the amino acid sequence identified by UniProtKB/Swiss-Prot P01116: Variantp.Gly12Asp. As used herein, a “KRas G13D inhibitor” refers to compounds of the present invention that are represented by Formula (I), as described herein. These compounds are capable of negatively modulating or inhibiting all or a portion of the enzymatic activity of KRas G13D. A “KRas G13D-associated disease or disorder” as used herein refers to diseases or disorders associated with or mediated by or having a KRas G13D mutation. A non-limiting example of a KRas G13D-associated disease or disorder is a KRas G13D-associated cancer.

As used herein, “KRas Q61H” refers to a mutant form of a mammalian KRas protein that contains an amino acid substitution of a histidine for a glutamine at amino acid position 61. The assignment of amino acid codon and residue positions for human KRas is based on the amino acid sequence identified by UniProtKB/Swiss-Prot P01116: Variantp.Gly12Asp. As used herein, a “KRas Q61H inhibitor” refers to compounds of the present invention that are represented by Formula (I), as described herein. These compounds are capable of negatively modulating or inhibiting all or a portion of the enzymatic activity of KRas Q61H. A “KRas Q61H-associated disease or disorder” as used herein refers to diseases or disorders associated with or mediated by or having a KRas Q61H mutation. A non-limiting example of a KRas Q61H-associated disease or disorder is a KRas Q61H-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having wild type KRas or a KRas G12A, G12C, G12D, G12R, G12S, G12V, G13D and/or Q61H mutation (e.g., as determined using a regulatory agency-approved, e.g., FDA-approved, assay or kit). In some embodiments, the subject has a tumor that is positive for wild type KRas or a KRas G12A, G12C, G12D, G12R, G12S, G12V, G13D and/or Q61H mutation (e.g., as determined using a regulatory agency-approved assay or kit). The subject can be a subject with a tumor(s) that is positive for wild type KRas or a KRas G12A, G12C, G12D, G12R, G12S, G12V, G13D and/or Q61H mutation (e.g., identified as positive using a regulatory agency-approved, e.g., FDA-approved, assay or kit). The subject can be a subject whose tumors have wild type KRas or a KRas G12A, G12C, G12D, G12R, G12S, G12V, G13D and/or Q61H mutation (e.g., where the tumor is identified as such using a regulatory agency-approved, e.g., FDA-approved, kit or assay). In some embodiments, the subject is suspected of having wild type KRas or a KRas G12A, KRas G12C, KRas G12D, KRas G12R, KRas G12S, KRas G12V, KRas G13D or KRas Q61H gene-associated cancer. In some embodiments, the subject has a clinical record indicating that the subject has a tumor that has wild type KRas or a KRas G12A, G12C, G12D, G12R, G12S, G12V, G13D and/or Q61H mutation (and optionally the clinical record indicates that the subject should be treated with any of the compositions provided herein).

In some embodiments of any of the methods or uses described herein, an assay is used to determine whether the patient has wild type KRas or a KRas G12A, G12C, G12D, G12R, G12S, G12V, G13D and/or Q61H mutation using a sample (e.g., a biological sample or a biopsy sample (e.g., a paraffin-embedded biopsy sample) from a patient (e.g., a patient suspected of having wild type KRas-associated or a KRas G12A, G12C, G12D, G12R, G12S, G12V, G13D and/or Q61H-associated cancer, a patient having one or more symptoms of wild type KRas-associated or a KRas G12A, G12C, G12D, G12R, G12S, G12V, G13D and/or Q61H-associated cancer, and/or a patient that has an increased risk of developing wild type KRas-associated or a KRas G12A, G12C, G12D, G12R, G12S, G12V, G13D and/or Q61H-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH₃.

The terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, propylene, and butylene.

The terms “C1-C3 alkoxy” and “C1-C4 alkoxy” refer to —OC1-C3 alkyl and —OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with one or more R⁸ or R′ groups as defined herein. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to —C1-C3 alkylene-OH and —C1-C4 alkylene-OH, respectively.

As used herein, the term “C2-C4 hydroxyalkynyl” refers to —C2-C4 alkynylene-OH.

An “aryl” group is a C₆-C₁₄ aromatic moiety comprising one to three aromatic rings, which is optionally substituted with one or more R⁸ or R⁹ groups as defined herein. As one embodiment, the aryl group is a C₆-C₁₀ aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the following ring system:

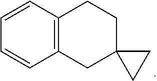 [SMILES: c1ccc2c(c1)CCC1(CC1)C2]

An “araC1-C6 alkyl” or “arylalkyl” group comprises an aryl group covalently linked to an alkyl group, either of which may independently be optionally substituted or unsubstituted. An example of an aralkyl group is (C6-C10) aryl(C1-C6)alkyl-, including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic or a bridged ring system. The heterocyclic group is optionally substituted with one or more R⁸ or R⁹ groups on ring carbon or ring nitrogen at one or more positions, wherein R⁶ is as defined for Formula I. The heterocyclic group is also independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14π electrons shared in a cyclic array; and having, in addition to carbon atoms, from one to three heteroatoms per ring, or from one to three heteroatoms in at least one ring, selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of one or more of wild type KRas, KRas G12A, KRas G12C, KRas G12D, KRas G12R, KRas G12S, KRas G12V, KRas G13D or KRas Q61H. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

In certain embodiments of the invention there are provided compounds of Formula (I):

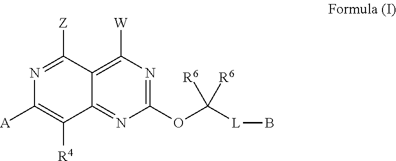

or a pharmaceutically acceptable salt thereof, wherein:
W is:

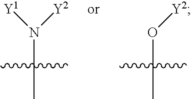

A is aryl or heteroaryl, wherein the aryl or the heteroaryl is optionally substituted with 1-4 R¹;
B is:

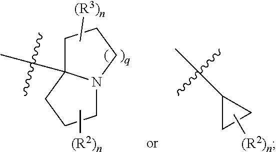

Y¹ is hydrogen, L-hydroxy optionally substituted with 1-4 R⁸, L-alkoxy optionally substituted with 1-4 R⁸, halogen, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle substituted with 1-2 oxo (═O) or oxo-containing substituent, and optionally further substituted with 1-2 R⁸,
Y² is hydrogen or C1-C4 alkyl;
or Y¹ and Y² join to form:

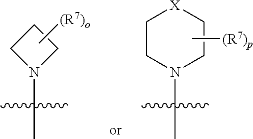

where X is selected from: a bond, —S—, —O—, —N<bound to a fused ring, —CH₂—, —CH₂—N—, —CH₂—N—CH₂—, —CH₂—CH₂—CH₂—, —CH₂—CH₂—, —O—CH₂— and —S—CH₂—;
or Y² and Z join to form V, where V is:

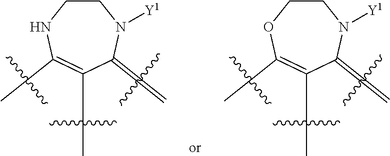

optionally substituted with 1-4 R⁸,
Z is hydrogen or joins with Y²;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, —CHR¹¹ or ═C(R¹¹)₂, or two R² join to form a heterocycle or cycloalkyl optionally substituted with C1-C3 alkyl;
each R³ is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, —CH₂, —CHR¹¹ or ═C(R¹¹)₂, or two R² join to form a heterocycle or cycloalkyl optionally substituted with C1-C3 alkyl;
wherein if V is not present at least one of R² and R³ are ═CH₂, —CHR¹¹ or ═C(R¹¹)₂;
each R⁴ is independently hydrogen, halogen or C1-C3 alkyl;
each R⁵ is independently hydrogen or C1-C3 alkyl, or two R⁵ join to form cycloalkyl or heterocycle;
each R⁶ is independently hydrogen, hydroxy, C1-C4 hydroxyalkyl or heteroaryl,
or two R⁶ join to form C3-C6 cycloalkyl or heterocycle;
each R⁷ is independently hydrogen, C1-C3 alkyl, C2 alkenyl, hydroxy, halogen, C1-C3 haloalkyl, -L-NH₂, —NH(C1-C3 alkyl), —N(C1-C3 alkyl)₂, oxo (═O), L-O—(C1-C3 alkyl), L-O—(C1-C3 alkyl)-OR⁵, —(C1-C3 alkyl)-OH, —C(O)OH, —C(O)O(C1-C3 alkyl), L-C(O)N(R¹⁰)₂, —NHC(O)H, —CN, aryl, —(CH₂)₁₋₂S(O)₂N(R¹⁰)₂, —NH—S(O)₂N(R¹⁰)₂, —O—S(O)₂N(R¹⁰)₂, S(O)₂R¹⁰, —P(O)(R⁵)₂ or L-heteroaryl or L-heterocycle optionally independently substituted with 1-2 substituents independently selected from C1-C3 alkyl, —CN and C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl, cyano and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a 1-2 carbon bridge;
each R⁸ is independently C1-C3 alkyl, hydroxy, halogen, —N(R¹⁰)₂, —N(R¹⁰)C(O)R¹⁰, oxo (═O), —O—(C1-C3 alkyl), —(C1-C3 alkyl)-OH, —C(O)OH, —C(O)N(R¹⁰)₂, —C(O)O(C1-C3 alkyl), —C(O)N(R¹⁰)₂, heteroaryl, heterocycle or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
each R¹⁰ is independently hydrogen, halogen, C1-C3 alkyl, C3-C4 cycloalkyl optionally substituted with 1-2 substituents independently selected from halogen and hydroxy, or two R¹⁰ join to form cycloalkyl or heterocycle optionally substituted with 1-2 C1-C3 alkyl;
each R¹¹ is independently halogen or methyl;
each L is independently a bond, —O—, —C1-C4 alkyl-, —C1-C4 alkyl-NH—, —NH—, —N(C1-C3 alkyl)- or cyclopropyl-CH₂—;
each n is 0-3;
is 1-6;
p is 1-8; and
q is 1-2.

In a preferred embodiment, R¹¹ is fluorine.

Embodiments of the invention also include compounds of Formula (IA):

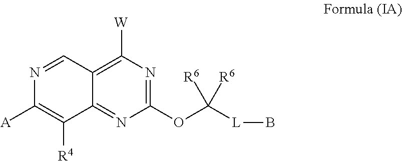

or a pharmaceutically acceptable salt thereof, wherein:
W is:

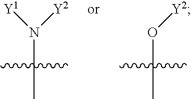

A is aryl or heteroaryl, wherein the aryl or the heteroaryl is optionally substituted with 1-4R¹;
B is:

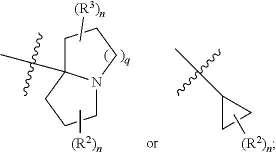

Y¹ is hydrogen, L-hydroxy optionally substituted with 1-4 R⁸, L-alkoxy optionally substituted with 1-4 R⁸, halogen, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle substituted with 1-2 oxo (═O) or oxo-containing substituent, and optionally further substituted with 1-2 R⁸,
Y² is hydrogen or C1-C4 alkyl;
or Y¹ and Y² join to form:

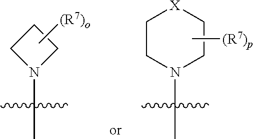

where X is selected from: a bond, —S—, —O—, —N<bound to a fused ring, —CH₂—, —CH₂—N—, —CH₂—N—CH₂—, —CH₂—CH₂—CH₂—, —CH₂—CH₂—, —O—CH₂— and —S—CH₂—;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
each R³ is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
each R⁴ is independently hydrogen, halogen or C1-C3 alkyl;
each R⁵ is independently hydrogen or C1-C3 alkyl, or two R⁵ join to form cycloalkyl or heterocycle;
each R⁶ is independently hydrogen, hydroxy, C1-C4 hydroxyalkyl or heteroaryl,
or two R⁶ join to form C3-C6 cycloalkyl or heterocycle;
each R⁷ is independently hydrogen, C1-C3 alkyl, C2 alkenyl, hydroxy, halogen, C1-C3 haloalkyl, -L-NH₂, —NH(C1-C3 alkyl), —N(C1-C3 alkyl)₂, oxo (═O), L-O—(C1-C3 alkyl), L-O—(C1-C3 alkyl)-OR⁵, —(C1-C3 alkyl)-OH, —C(O)OH, —C(O)O(C1-C3 alkyl), L-C(O)N(R¹⁰)₂, —NHC(O)H, —CN, aryl, —(CH₂)₁₋₂S(O)₂N(R¹⁰)₂, —NH—S(O)₂N(R¹⁰)₂, —O—S(O)₂N(R¹⁰)₂, S(O)₂R¹⁰, —P(O)(R⁵)₂ or L-heteroaryl or L-heterocycle optionally independently substituted with 1-2 substituents independently selected from C1-C3 alkyl, —CN and C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl, cyano and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a 1-2 carbon bridge;
each R⁸ is independently C1-C3 alkyl, hydroxy, halogen, —N(R¹⁰)₂, —N(R¹⁰)C(O)R¹⁰, oxo (═O), —O—(C1-C3 alkyl), —(C1-C3 alkyl)-OH, —C(O)OH, —C(O)N(R¹⁰)₂, —C(O)O(C1-C3 alkyl), —C(O)N(R¹⁰)₂, heteroaryl, heterocycle or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
each R¹⁰ is independently hydrogen, halogen, C1-C3 alkyl, C3-C4 cycloalkyl optionally substituted with 1-2 substituents independently selected from halogen and hydroxy, or two R¹⁰ join to form cycloalkyl or heterocycle optionally substituted with 1-2 C1-C3 alkyl;
each R¹¹ is independently halogen or methyl;
each L is independently a bond, —O—, —C1-C4 alkyl-, —C1-C4 alkyl-NH—, —NH—, —N(C1-C3 alkyl)- or cyclopropyl-CH₂—;
each n is 0-3;
is 1-6;
p is 1-8; and
q is 1-2.

Embodiments also include such compounds or salts wherein each R¹ is independently selected from halogen, hydroxy, C1-C3 alkoxy and C1-C4 alkyl.

Embodiments also include such compounds or salts wherein each R², if present, is selected from hydrogen and halogen, and wherein each R³, if present, is selected from hydrogen and halogen.

Embodiments also include such compounds or salts wherein each R⁷ is independently selected from hydrogen, C1-C4 alkyl, hydroxy, C1-C3 alkoxy, and wherein two R⁷ on non-adjacent atoms optionally join to form a 1-2 carbon bridge.

Embodiments also include such compounds or salts wherein each R⁶ is independently hydrogen or hydroxy.

Embodiments also include such compounds or salts wherein B is:

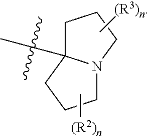

Embodiments also include such compounds or salts wherein Y¹ and Y² join to form:

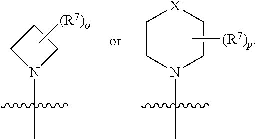

Embodiments also include such compounds or salts wherein Y¹ and Y² join to form:

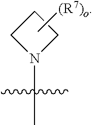

Embodiments also include such compounds or salts wherein Y¹ and Y² join to form:

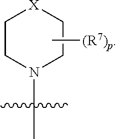

Embodiments also include such compounds or salts wherein A is naphthyl.

Embodiments also include such compounds or salts wherein A is indazolyl.

Embodiments also include such compounds or salts wherein A is phenyl.

Embodiments also include such compounds or salts wherein A is pyridyl.

In certain embodiments of the invention at least one R¹ is C1-C4 alkyl.

In certain embodiments of the invention at least one R¹ is halogen, preferably fluorine or chlorine.

In certain embodiments of the invention at least one R¹ is hydroxy

In certain embodiments of the invention at least one R² is C1-C4 alkyl.

In certain embodiments of the invention at least one R² is halogen, preferably fluorine or chlorine.

In certain embodiments of the invention at least one R² is hydroxy.

In certain embodiments of the invention at least one R² is ═CH₂, ═CHR¹¹ or ═C(R¹¹)₂.

In certain embodiments of the invention at least one R³ is C1-C4 alkyl.

In certain embodiments of the invention at least one R³ is halogen, preferably fluorine or chlorine.

In certain embodiments of the invention at least one R³ is hydroxy.

In certain embodiments of the invention at least one R³ is ═CH₂, ═CHR¹¹ or ═C(R¹¹)₂.

In certain embodiments of the invention R⁴ is halogen, preferably fluorine.

In certain embodiments of the invention at least one R⁵ is C1-C4 alkyl.

In certain embodiments of the invention at least one R⁵ is hydrogen.

In certain embodiments of the invention at least one R⁶ is C1-C4 alkyl.

In certain embodiments of the invention, two R⁶ join to form C3-C6 cycloalkyl or heterocycle.

In certain embodiments of the invention at least one R⁶ is hydrogen.

In certain embodiments of the invention both R⁶ are C1-C4 alkyl.

In certain embodiments of the invention both R⁶ are hydrogen.

In certain embodiments Y¹ is L-C3-C6 cycloalkyl, L-heteroaryl, L-aryl, or L-heterocycle. In certain of these embodiments, L is a bond. In certain of these embodiments L is C1-C4 alkyl. In certain of these embodiments L is NH or N(C1-C3)alkyl.

In certain embodiments Y¹ is L-heteroaryl where the heteroaryl is thietane dioxide, iso-thiazolidine dioxide, imidazopyrazine, pyridine or pyrimidine.

In certain embodiments Y¹ is L-C3-C6 cycloalkyl where the cycloalkyl is preferably cyclobutane, cyclopentane, cyclohexane or cycloheptane.

In certain embodiments Y¹ is L-heterocycle where the heterocycle is preferably pyrrolidinone.

In certain embodiments of the invention Y² is hydrogen.

In certain embodiments of the invention Y² is C1-C4 alkyl;

In certain embodiments of the invention at least one R⁸ is C1-C4 alkyl, preferably methyl.

In certain embodiments of the invention at least one R⁸ is hydroxy or C1-C3 alkyl-hydroxy.

In certain embodiments of the invention one or two R⁸ is oxo (═O).

In certain embodiments of the invention at least one R⁸ is aryl or heteroaryl.

In certain embodiments of the invention at least one R⁸ is C(O)OH.

In certain embodiments of the invention at least one R⁸ is —C(O)NH₂, —C(O)NH(C1-C3 alkyl) or —C(O)N(C1-C3 alkyl)₂.

In certain embodiments of the invention R⁸ is —NH₂, —NH(C1-C3 alkyl); —N(C1-C3 alkyl)₂.

In certain embodiments of the invention at least one R⁹ is C1-C4 alkyl, preferably methyl.

In certain embodiments of the invention at least one R⁹ is hydroxy or C1-C3 alkyl-hydroxy.

In certain embodiments of the invention one or two R⁹ is oxo (═O).

In certain embodiments of the invention at least one R⁹ is aryl or heteroaryl.

In certain embodiments of the invention at least one R⁹ is C(O)OH.

In certain embodiments of the invention at least one R⁹ is —C(O)NH₂, —C(O)NH(C1-C3 alkyl) or —C(O)N(C1-C3 alkyl)₂.

In certain embodiments of the invention at least one R¹¹ is F.

In certain embodiments of the invention Y¹ and Y² join to form a piperidine, azepane, azocane, thiazepine, diazepane, oxazepane, azetidine, pyrrolidine, piperazine bound to a fused ring via nitrogen or thiomorpholine.

In certain embodiments of the invention, two R⁷ on the same atom join to form a spirocyclic ring selected from C3-C6 cycloalkyl and heterocycle, where said spirocyclic ring is optionally substituted with oxo (═O), halogen, hydroxy, C1-C3 alkyl and —O—(C1-C3 alkyl).

In certain embodiments of the invention, two R⁷ on adjacent atoms optionally join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R⁸.

In certain embodiments of the invention, two R⁷ on non-adjacent atoms optionally join to form a 1-2 carbon bridge.

Nonlimiting examples of the compounds of the invention include:

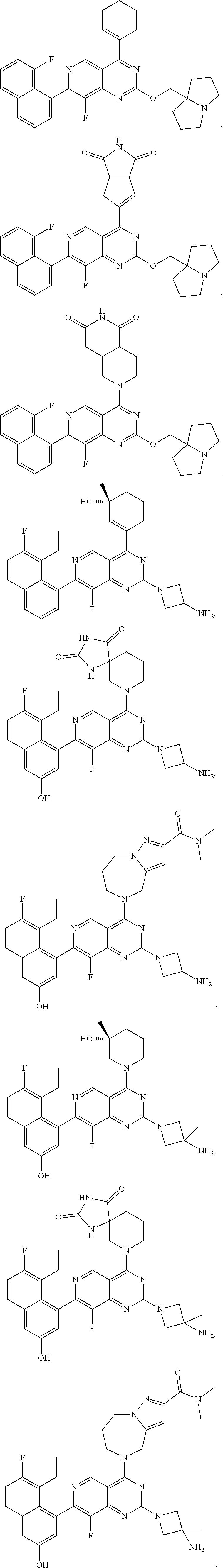

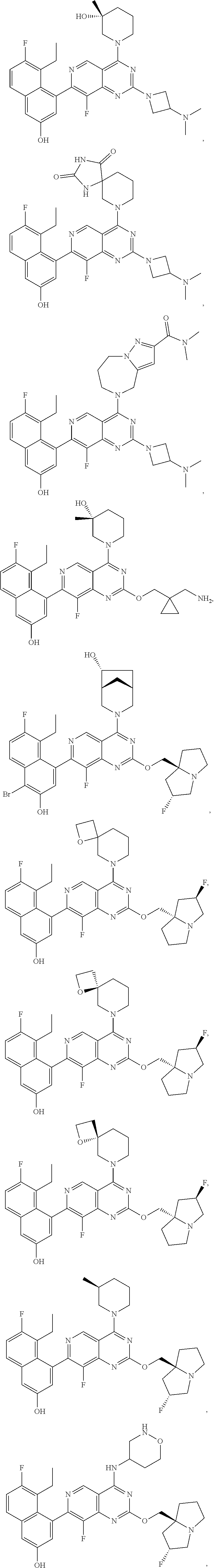

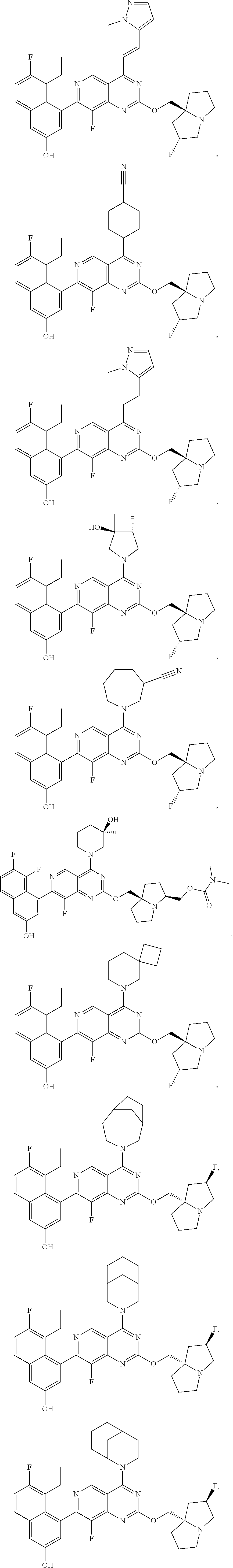

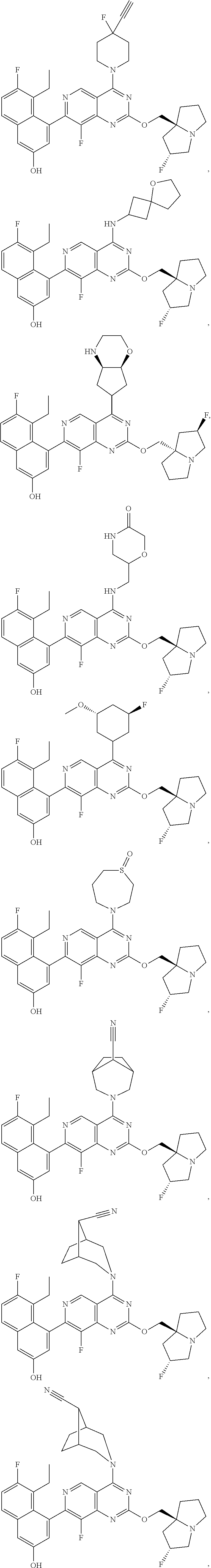

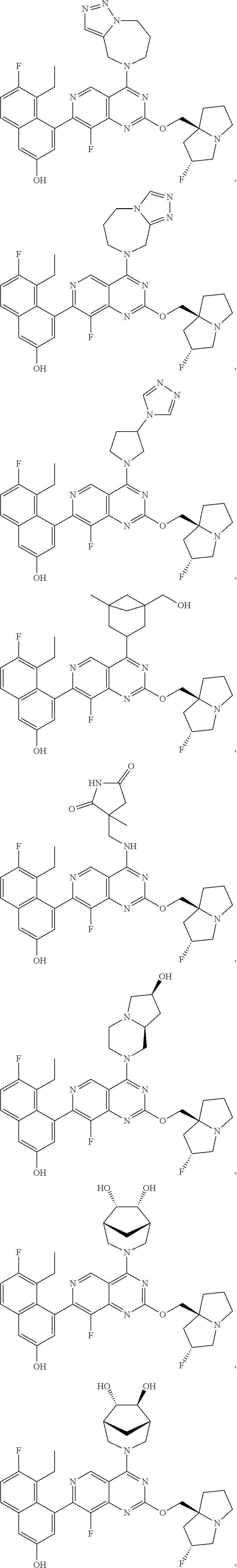

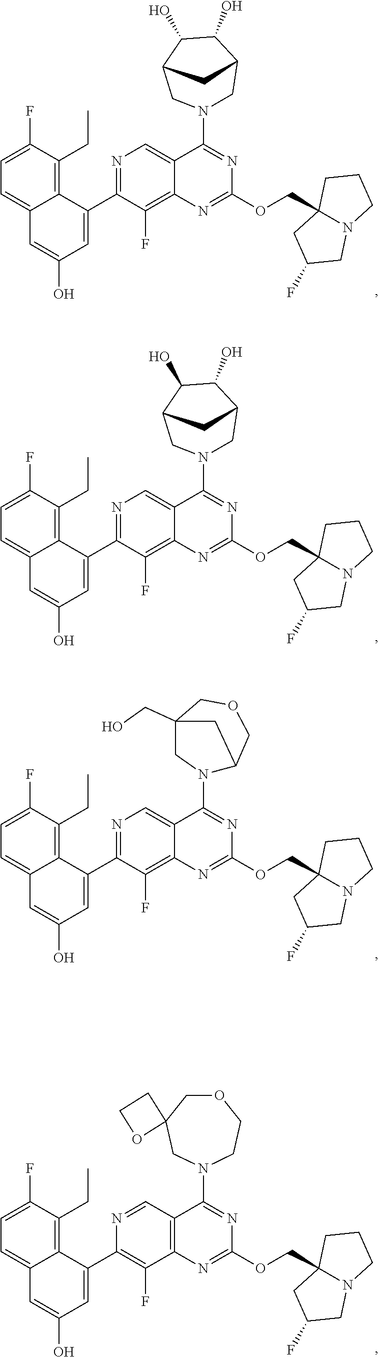 [SMILES: CCc1c(F)ccc2cc(O)cc(-c3ncc4c(N5C[C@@H]6C[C@H](C5)[C@H](O)[C@@H]6O)nc(OC[C@@]56CCCN5C[C@H](F)C6)nc4c3F)c12 ; CCc1c(F)ccc2cc(O)cc(-c3ncc4c(N5C[C@H]6C[C@@H](C5)[C@@H](O)[C@@H]6O)nc(OC[C@@]56CCCN5C[C@H](F)C6)nc4c3F)c12 ; CCc1c(F)ccc2cc(O)cc(-c3ncc4c(N5CC6(CO)COCC5C6)nc(OC[C@@]56CCCN5C[C@H](F)C6)nc4c3F)c12 ; CCc1c(F)ccc2cc(O)cc(-c3ncc4c(N5CCOCC6(CCO6)C5)nc(OC[C@@]56CCCN5C[C@H](F)C6)nc4c3F)c12]

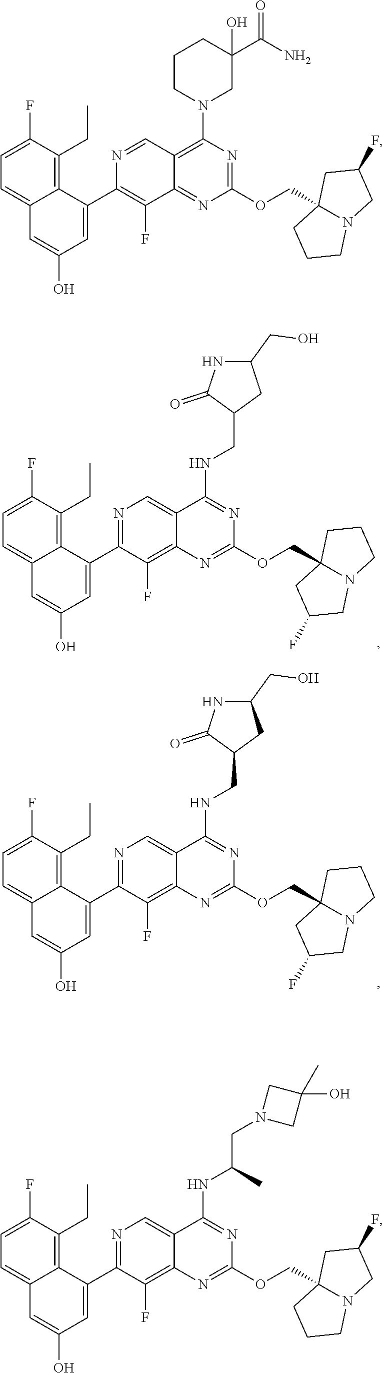

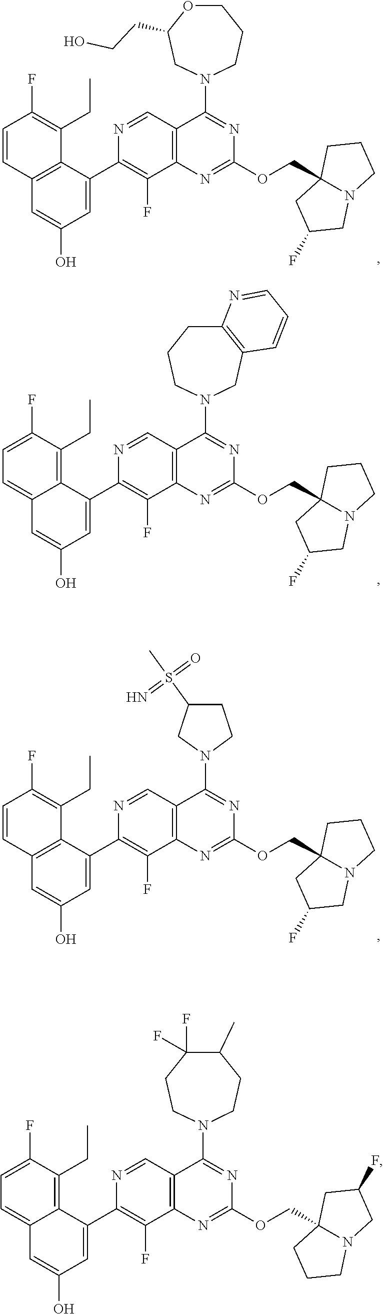

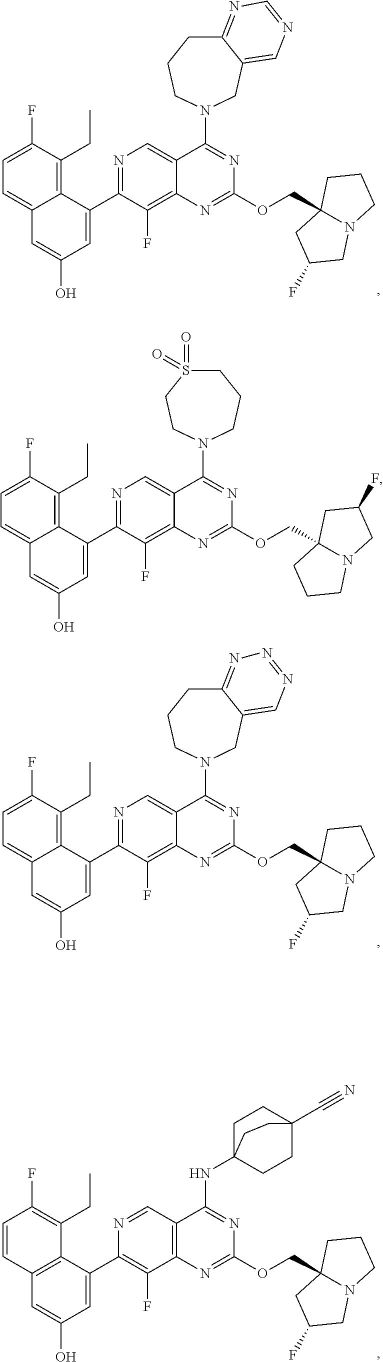 [SMILES: CCc1c(F)ccc2cc(O)cc(-c3ncc4c(N5CCCc6ncncc6C5)nc(OC[C@@]56CCCN5C[C@H](F)C6)nc4c3F)c12 ; CCc1c(F)ccc2cc(O)cc(-c3ncc4c(N5CCCS(=O)(=O)CC5)nc(OC[C@@]56CCCN5C[C@H](F)C6)nc4c3F)c12 ; CCc1c(F)ccc2cc(O)cc(-c3ncc4c(N5CCCc6nnncc6C5)nc(OC[C@@]56CCCN5C[C@H](F)C6)nc4c3F)c12 ; CCc1c(F)ccc2cc(O)cc(-c3ncc4c(NC56CCC(C#N)(CC5)CC6)nc(OC[C@@]56CCCN5C[C@H](F)C6)nc4c3F)c12]

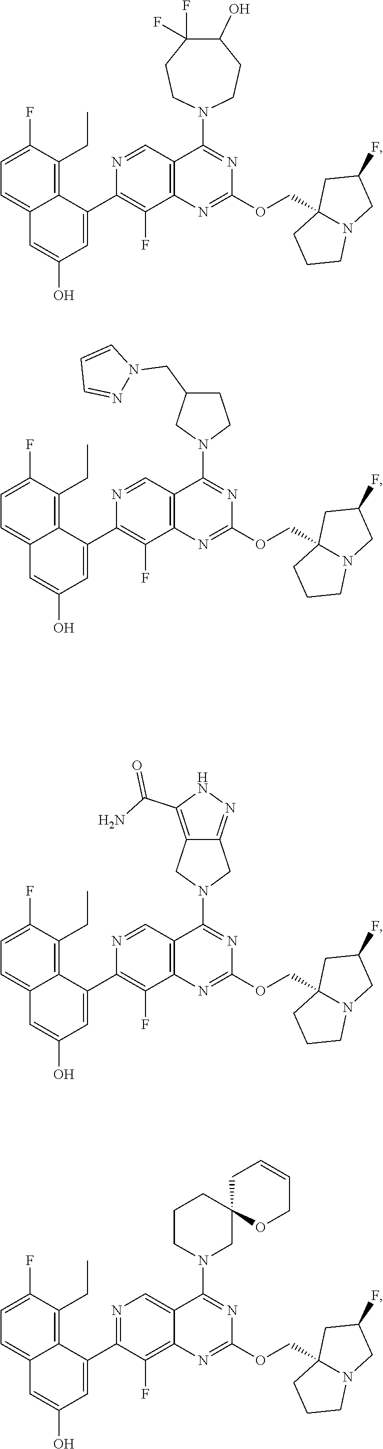 [SMILES: CCc1c(F)ccc2cc(O)cc(-c3ncc4c(N5CCC(O)C(F)(F)CC5)nc(OC[C@@]56CCCN5C[C@H](F)C6)nc4c3F)c12 ; CCc1c(F)ccc2cc(O)cc(-c3ncc4c(N5CCC(Cn6cccn6)C5)nc(OC[C@@]56CCCN5C[C@H](F)C6)nc4c3F)c12 ; CCc1c(F)ccc2cc(O)cc(-c3ncc4c(N5Cc6n[nH]c(C(N)=O)c6C5)nc(OC[C@@]56CCCN5C[C@H](F)C6)nc4c3F)c12 ; CCc1c(F)ccc2cc(O)cc(-c3ncc4c(N5CCC[C@]6(CC=CCO6)C5)nc(OC[C@@]56CCCN5C[C@H](F)C6)nc4c3F)c12]

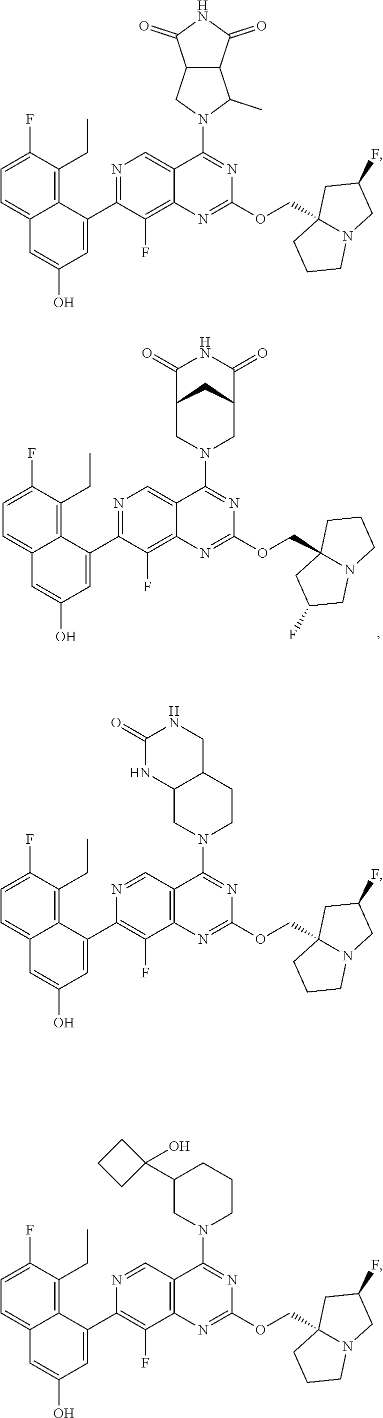 [SMILES: CCc1c(F)ccc2cc(O)cc(-c3ncc4c(N5CC6C(=O)NC(=O)C6C5C)nc(OC[C@@]56CCCN5C[C@H](F)C6)nc4c3F)c12 ; CCc1c(F)ccc2cc(O)cc(-c3ncc4c(N5C[C@H]6C[C@@H](C5)C(=O)NC6=O)nc(OC[C@@]56CCCN5C[C@H](F)C6)nc4c3F)c12 ; CCc1c(F)ccc2cc(O)cc(-c3ncc4c(N5CCC6CNC(=O)NC6C5)nc(OC[C@@]56CCCN5C[C@H](F)C6)nc4c3F)c12 ; CCc1c(F)ccc2cc(O)cc(-c3ncc4c(N5CCCC(C6(O)CCC6)C5)nc(OC[C@@]56CCCN5C[C@H](F)C6)nc4c3F)c12]

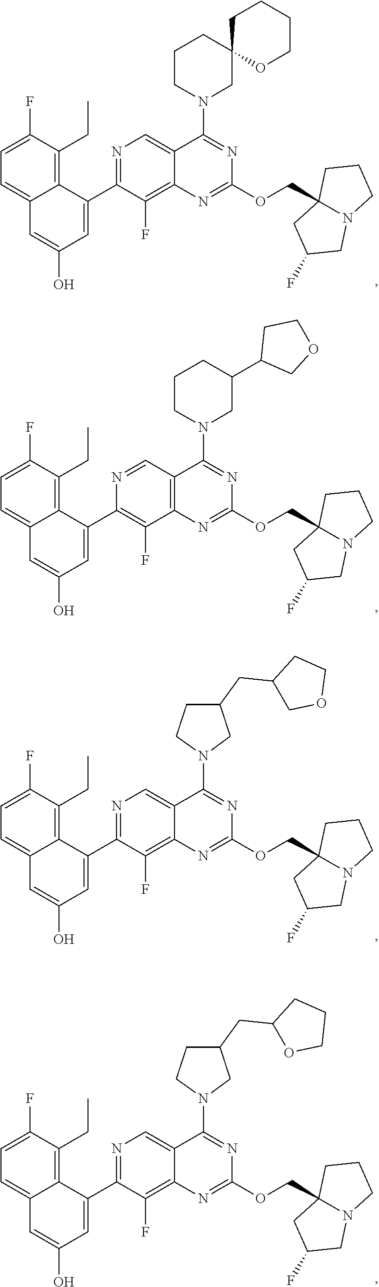

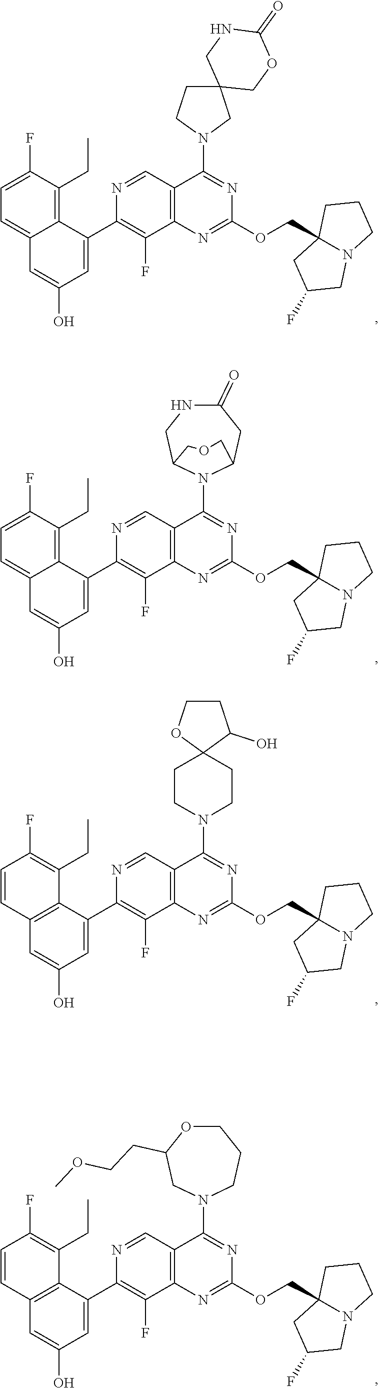 [SMILES: CCc1c(F)ccc2cc(O)cc(-c3ncc4c(N5CCC6(CNC(=O)OC6)C5)nc(OC[C@@]56CCCN5C[C@H](F)C6)nc4c3F)c12 ; CCc1c(F)ccc2cc(O)cc(-c3ncc4c(N5C6CNC(=O)CC5COC6)nc(OC[C@@]56CCCN5C[C@H](F)C6)nc4c3F)c12 ; CCc1c(F)ccc2cc(O)cc(-c3ncc4c(N5CCC6(CC5)OCCC6O)nc(OC[C@@]56CCCN5C[C@H](F)C6)nc4c3F)c12 ; CCc1c(F)ccc2cc(O)cc(-c3ncc4c(N5CCCOC(CCOC)C5)nc(OC[C@@]56CCCN5C[C@H](F)C6)nc4c3F)c12]

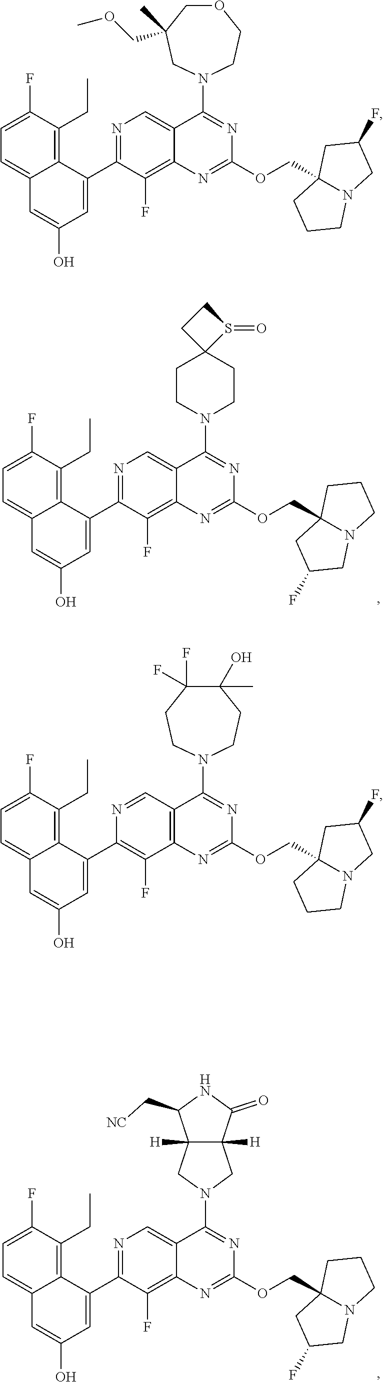 [SMILES: CCc1c(F)ccc2cc(O)cc(-c3ncc4c(N5CCOC[C@@](C)(COC)C5)nc(OC[C@@]56CCCN5C[C@H](F)C6)nc4c3F)c12 ; CCc1c(F)ccc2cc(O)cc(-c3ncc4c(N5CCC6(CC5)CC[S@]6=O)nc(OC[C@@]56CCCN5C[C@H](F)C6)nc4c3F)c12 ; CCc1c(F)ccc2cc(O)cc(-c3ncc4c(N5CCC(C)(O)C(F)(F)CC5)nc(OC[C@@]56CCCN5C[C@H](F)C6)nc4c3F)c12 ; CCc1c(F)ccc2cc(O)cc(-c3ncc4c(N5C[C@@H]6[C@@H](CC#N)NC(=O)[C@@H]6C5)nc(OC[C@@]56CCCN5C[C@H](F)C6)nc4c3F)c12]

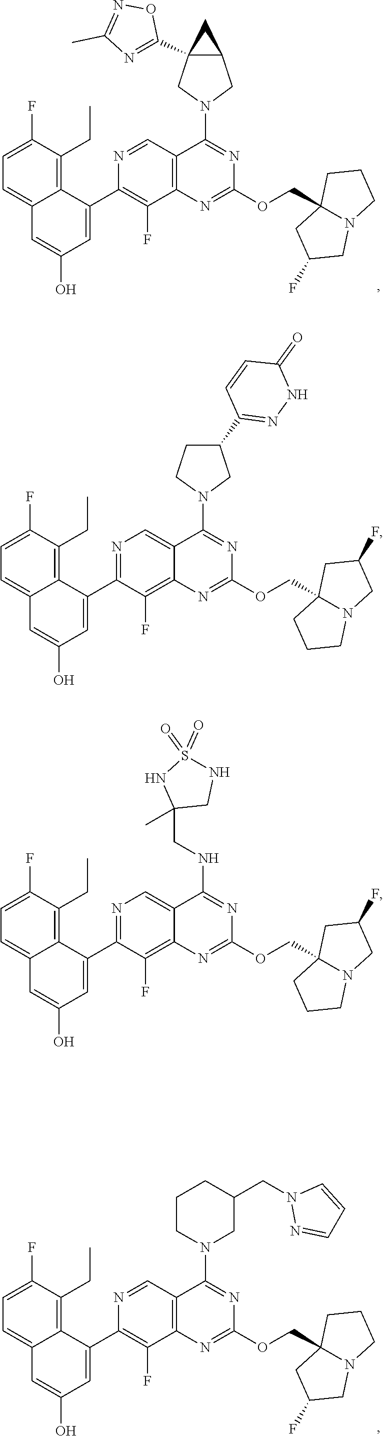 [SMILES: CCc1c(F)ccc2cc(O)cc(-c3ncc4c(N5C[C@@H]6C[C@]6(c6nc(C)no6)C5)nc(OC[C@@]56CCCN5C[C@H](F)C6)nc4c3F)c12 ; CCc1c(F)ccc2cc(O)cc(-c3ncc4c(N5CC[C@H](c6ccc(=O)[nH]n6)C5)nc(OC[C@@]56CCCN5C[C@H](F)C6)nc4c3F)c12 ; CCc1c(F)ccc2cc(O)cc(-c3ncc4c(NCC5(C)CNS(=O)(=O)N5)nc(OC[C@@]56CCCN5C[C@H](F)C6)nc4c3F)c12 ; CCc1c(F)ccc2cc(O)cc(-c3ncc4c(N5CCCC(Cn6cccn6)C5)nc(OC[C@@]56CCCN5C[C@H](F)C6)nc4c3F)c12]

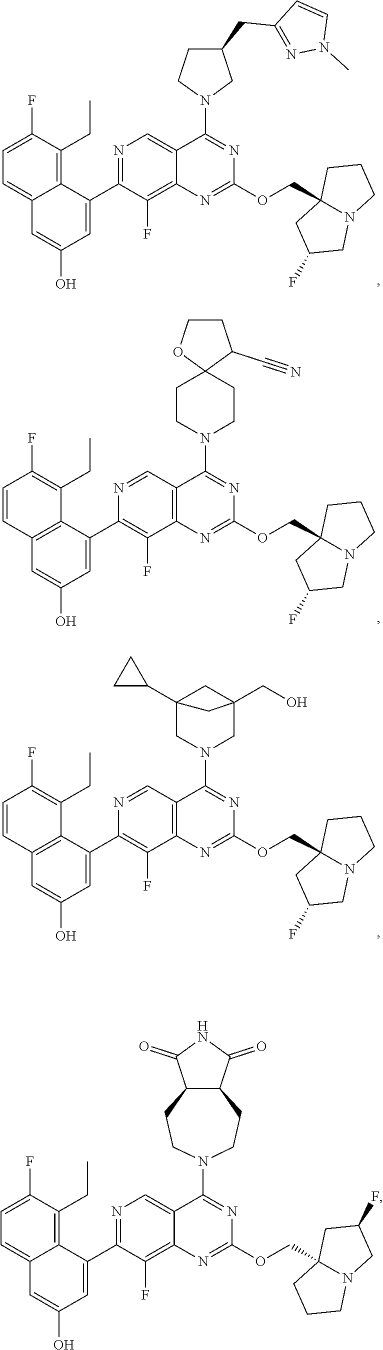 [SMILES: CCc1c(F)ccc2cc(O)cc(-c3ncc4c(N5CC[C@@H](Cc6ccn(C)n6)C5)nc(OC[C@@]56CCCN5C[C@H](F)C6)nc4c3F)c12 ; CCc1c(F)ccc2cc(O)cc(-c3ncc4c(N5CCC6(CC5)OCCC6C#N)nc(OC[C@@]56CCCN5C[C@H](F)C6)nc4c3F)c12 ; CCc1c(F)ccc2cc(O)cc(-c3ncc4c(N5CC6(CO)CC(C7CC7)(C5)C6)nc(OC[C@@]56CCCN5C[C@H](F)C6)nc4c3F)c12 ; CCc1c(F)ccc2cc(O)cc(-c3ncc4c(N5CC[C@@H]6C(=O)NC(=O)[C@@H]6CC5)nc(OC[C@@]56CCCN5C[C@H](F)C6)nc4c3F)c12]

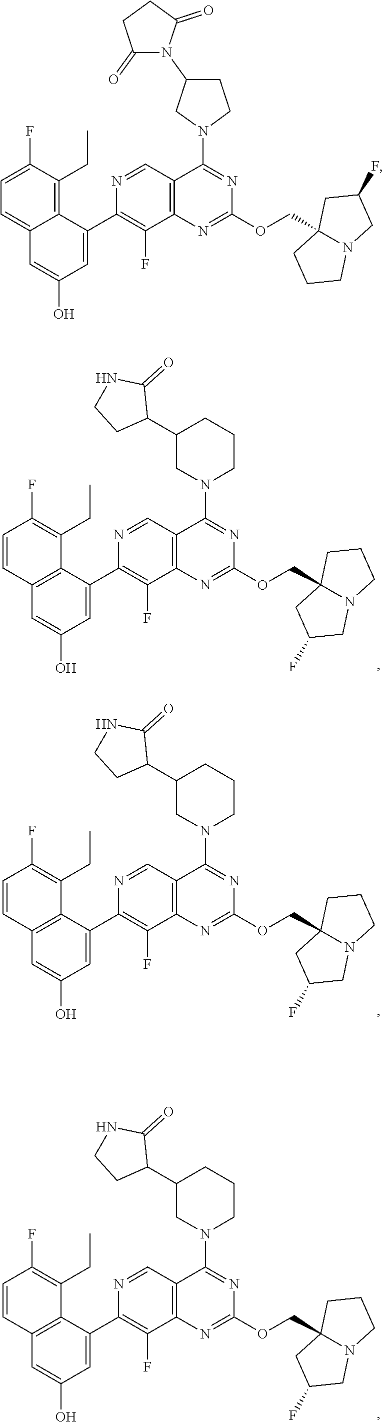 [SMILES: CCc1c(F)ccc2cc(O)cc(-c3ncc4c(N5CCC(N6C(=O)CCC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12]

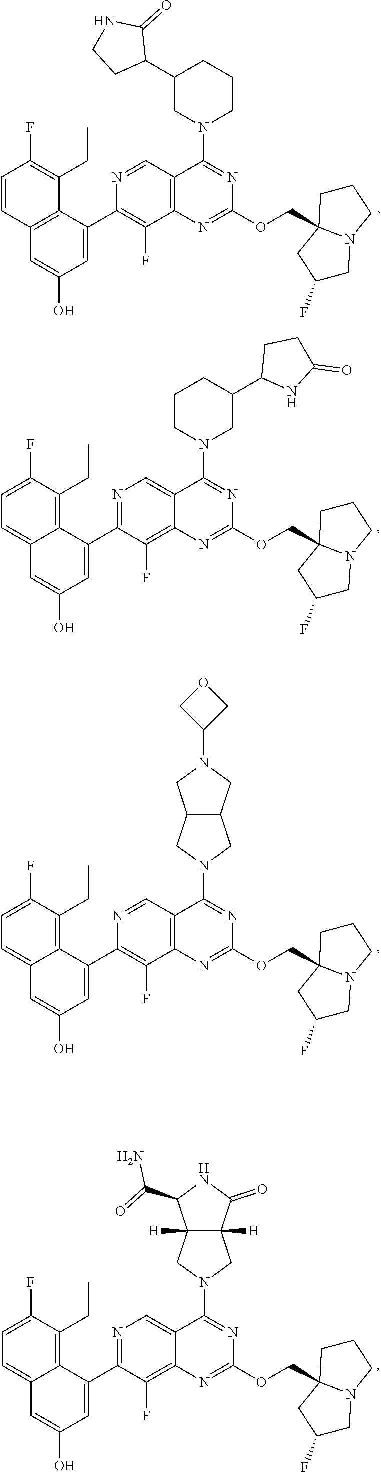

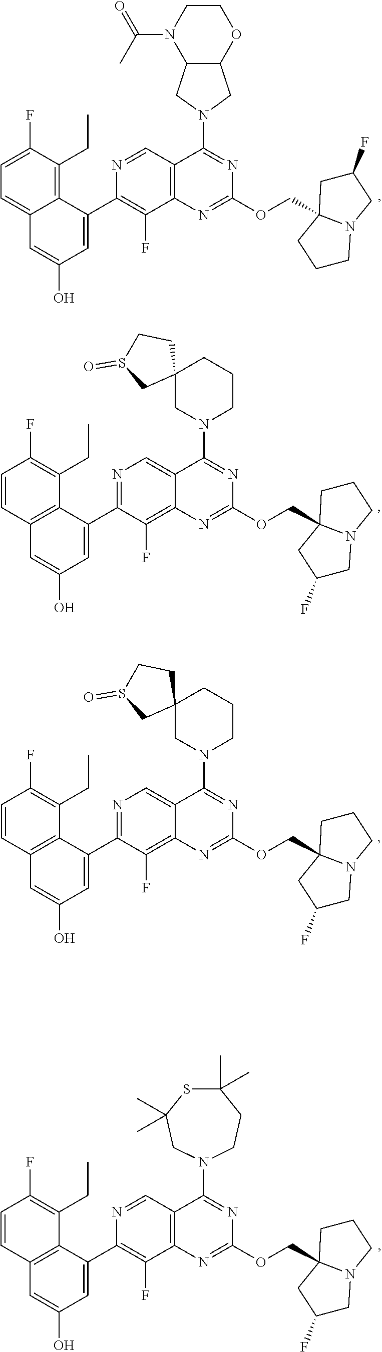 [SMILES: CCc1c(F)ccc2cc(O)cc(-c3ncc4c(N5CC6OCCN(C(C)=O)C6C5)nc(OC[C@@]56CCCN5C[C@H](F)C6)nc4c3F)c12 ; CCc1c(F)ccc2cc(O)cc(-c3ncc4c(N5CCC[C@@]6(CC[S@](=O)C6)C5)nc(OC[C@@]56CCCN5C[C@H](F)C6)nc4c3F)c12 ; CCc1c(F)ccc2cc(O)cc(-c3ncc4c(N5CCC[C@]6(CC[S@](=O)C6)C5)nc(OC[C@@]56CCCN5C[C@H](F)C6)nc4c3F)c12 ; CCc1c(F)ccc2cc(O)cc(-c3ncc4c(N5CCC(C)(C)SC(C)(C)C5)nc(OC[C@@]56CCCN5C[C@H](F)C6)nc4c3F)c12]

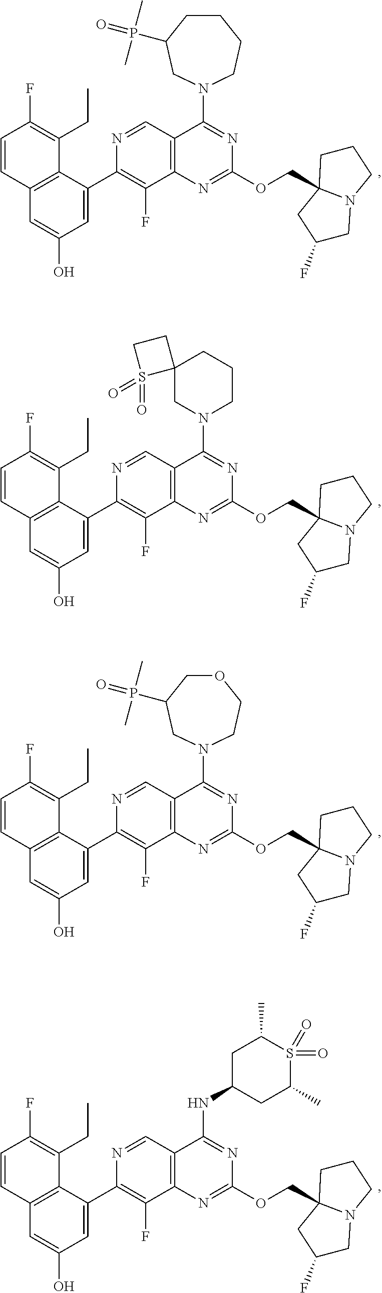 [SMILES: CCc1c(F)ccc2cc(O)cc(-c3ncc4c(N5CCCCC(P(C)(C)=O)C5)nc(OC[C@@]56CCCN5C[C@H](F)C6)nc4c3F)c12 ; CCc1c(F)ccc2cc(O)cc(-c3ncc4c(N5CCCC6(CCS6(=O)=O)C5)nc(OC[C@@]56CCCN5C[C@H](F)C6)nc4c3F)c12 ; CCc1c(F)ccc2cc(O)cc(-c3ncc4c(N5CCOCC(P(C)(C)=O)C5)nc(OC[C@@]56CCCN5C[C@H](F)C6)nc4c3F)c12 ; CCc1c(F)ccc2cc(O)cc(-c3ncc4c(N[C@@H]5C[C@@H](C)S(=O)(=O)[C@@H](C)C5)nc(OC[C@@]56CCCN5C[C@H](F)C6)nc4c3F)c12]

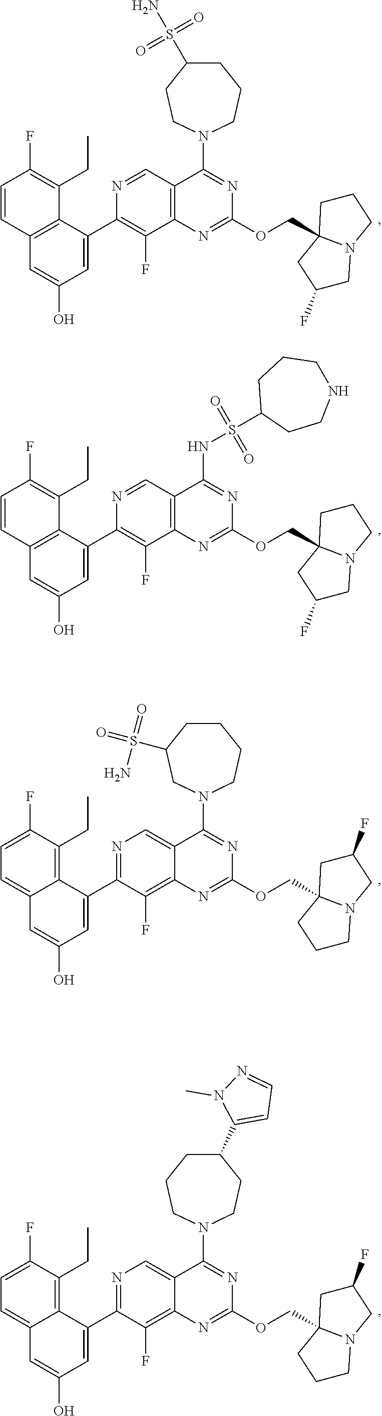

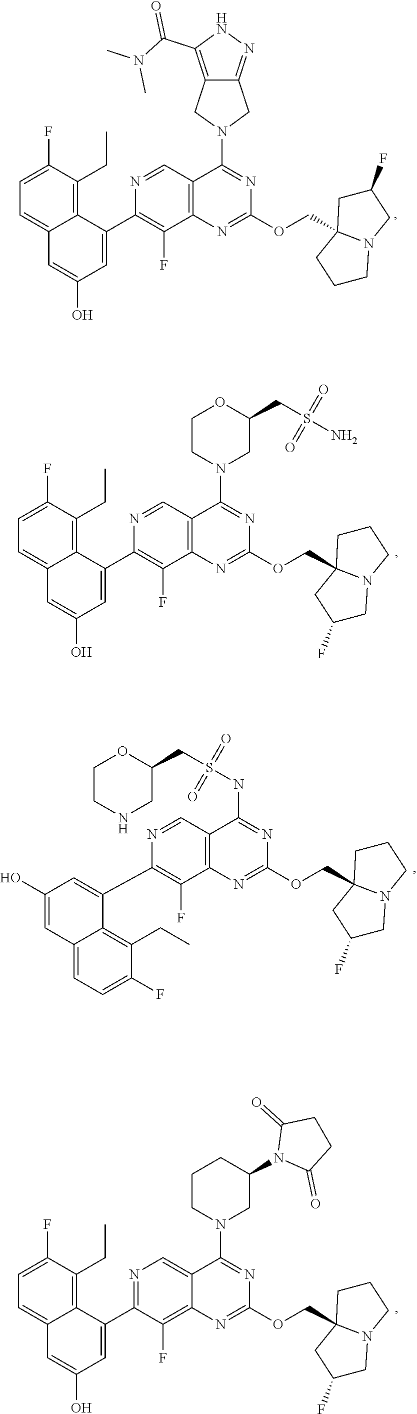 [SMILES: CCc1c(F)ccc2cc(O)cc(-c3ncc4c(N5Cc6n[nH]c(C(=O)N(C)C)c6C5)nc(OC[C@@]56CCCN5C[C@H](F)C6)nc4c3F)c12 ; CCc1c(F)ccc2cc(O)cc(-c3ncc4c(N5CCO[C@@H](CS(N)(=O)=O)C5)nc(OC[C@@]56CCCN5C[C@H](F)C6)nc4c3F)c12 ; CCc1c(F)ccc2cc(O)cc(-c3ncc4c(NS(=O)(=O)C[C@H]5CNCCO5)nc(OC[C@@]56CCCN5C[C@H](F)C6)nc4c3F)c12 ; CCc1c(F)ccc2cc(O)cc(-c3ncc4c(N5CCC[C@@H](N6C(=O)CCC6=O)C5)nc(OC[C@@]56CCCN5C[C@H](F)C6)nc4c3F)c12]

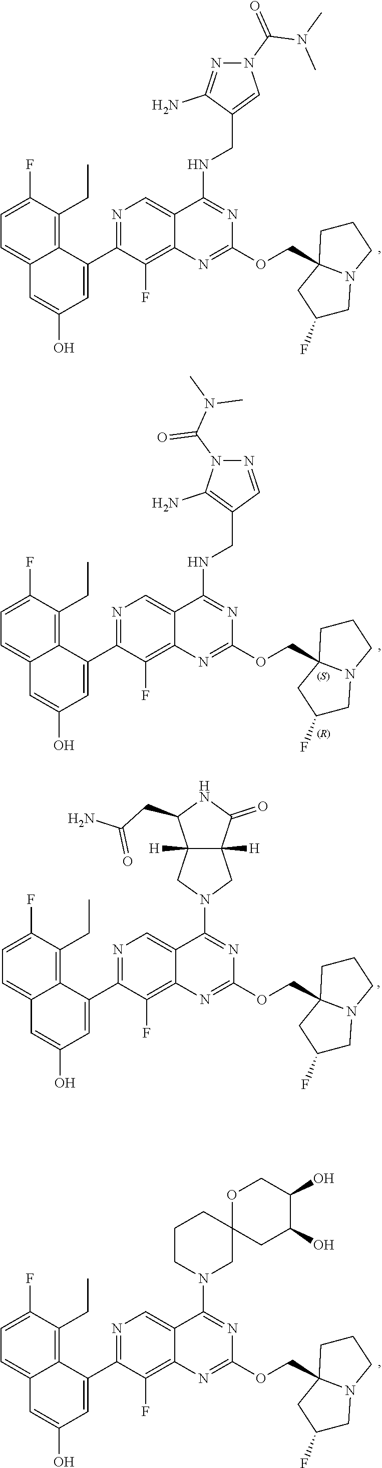

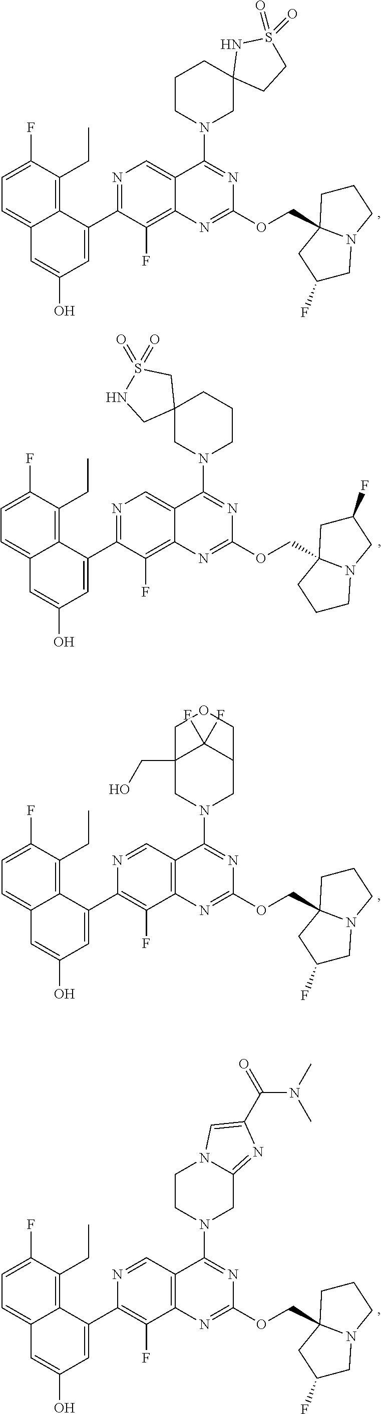 [SMILES: CCc1c(F)ccc2cc(O)cc(-c3ncc4c(N5CCCC6(CCS(=O)(=O)N6)C5)nc(OC[C@@]56CCCN5C[C@H](F)C6)nc4c3F)c12 ; CCc1c(F)ccc2cc(O)cc(-c3ncc4c(N5CCCC6(CNS(=O)(=O)C6)C5)nc(OC[C@@]56CCCN5C[C@H](F)C6)nc4c3F)c12 ; CCc1c(F)ccc2cc(O)cc(-c3ncc4c(N5CC6COCC(CO)(C5)C6(F)F)nc(OC[C@@]56CCCN5C[C@H](F)C6)nc4c3F)c12 ; CCc1c(F)ccc2cc(O)cc(-c3ncc4c(N5CCn6cc(C(=O)N(C)C)nc6C5)nc(OC[C@@]56CCCN5C[C@H](F)C6)nc4c3F)c12]

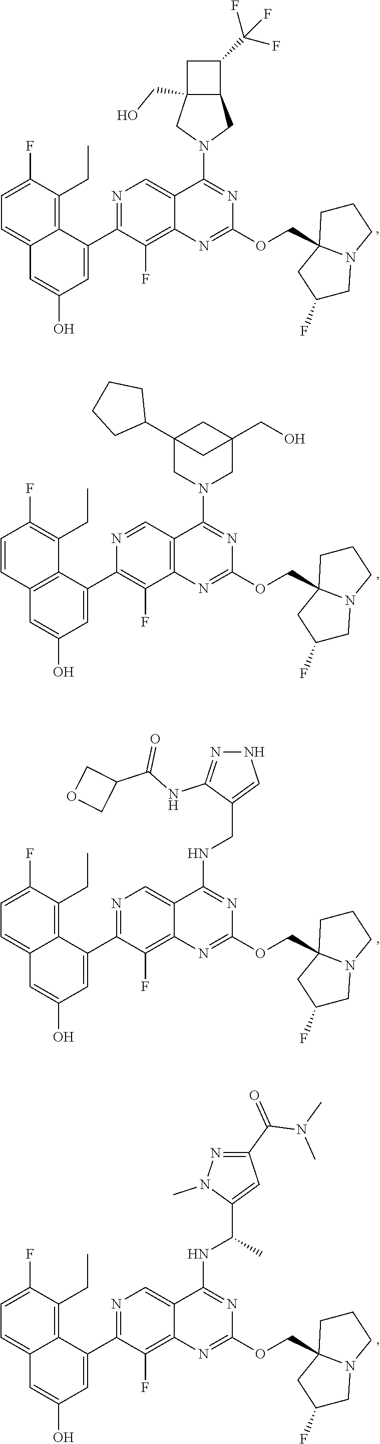 [SMILES: CCc1c(F)ccc2cc(O)cc(-c3ncc4c(N5C[C@@H]6[C@@H](C(F)(F)F)C[C@]6(CO)C5)nc(OC[C@@]56CCCN5C[C@H](F)C6)nc4c3F)c12 ; CCc1c(F)ccc2cc(O)cc(-c3ncc4c(N5CC6(CO)CC(C7CCCC7)(C5)C6)nc(OC[C@@]56CCCN5C[C@H](F)C6)nc4c3F)c12 ; CCc1c(F)ccc2cc(O)cc(-c3ncc4c(NCc5c[nH]nc5NC(=O)C5COC5)nc(OC[C@@]56CCCN5C[C@H](F)C6)nc4c3F)c12 ; CCc1c(F)ccc2cc(O)cc(-c3ncc4c(N[C@@H](C)c5cc(C(=O)N(C)C)nn5C)nc(OC[C@@]56CCCN5C[C@H](F)C6)nc4c3F)c12]

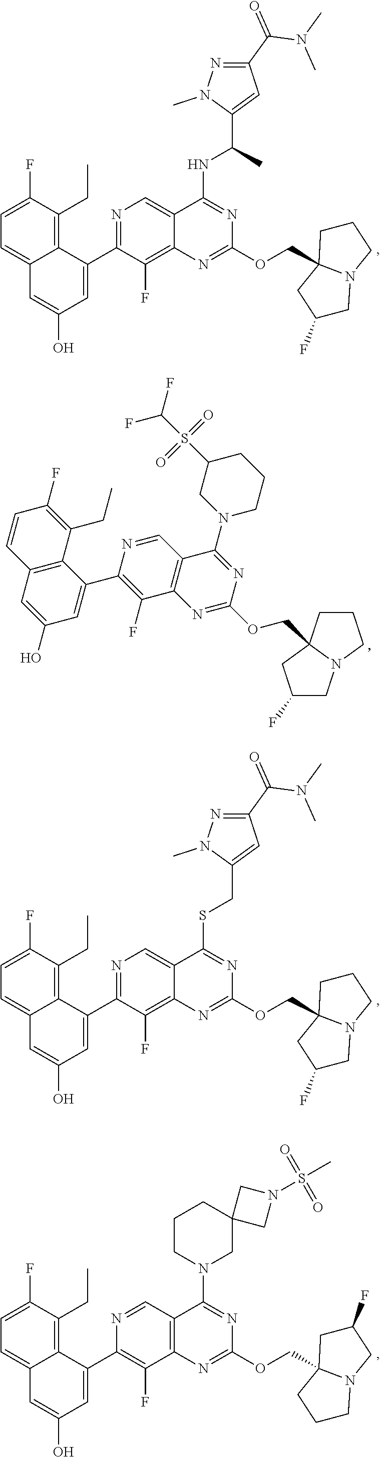

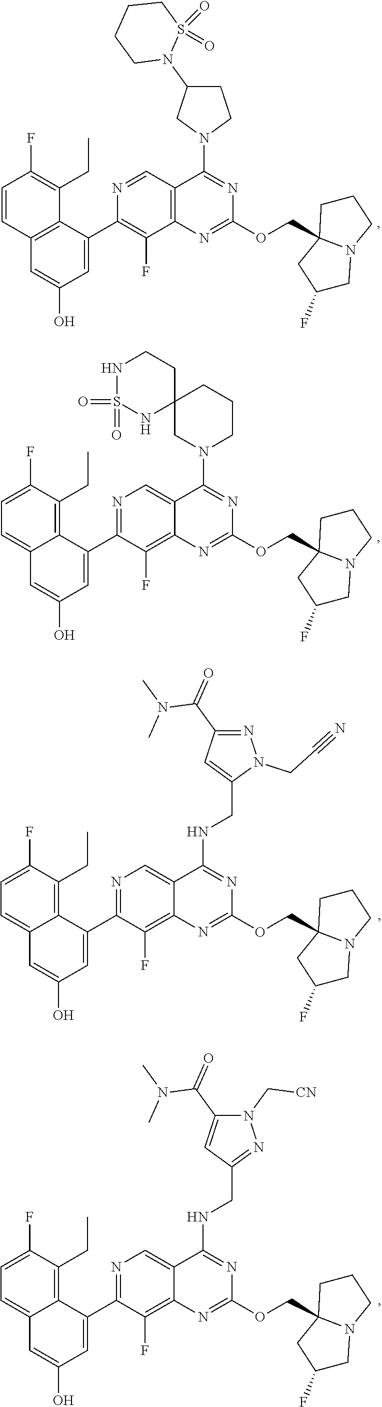 [SMILES: CCc1c(F)ccc2cc(O)cc(-c3ncc4c(N5CCC(N6CCCCS6(=O)=O)C5)nc(OC[C@@]56CCCN5C[C@H](F)C6)nc4c3F)c12 ; CCc1c(F)ccc2cc(O)cc(-c3ncc4c(N5CCCC6(CCNS(=O)(=O)N6)C5)nc(OC[C@@]56CCCN5C[C@H](F)C6)nc4c3F)c12 ; CCc1c(F)ccc2cc(O)cc(-c3ncc4c(NCc5cc(C(=O)N(C)C)nn5CC#N)nc(OC[C@@]56CCCN5C[C@H](F)C6)nc4c3F)c12 ; CCc1c(F)ccc2cc(O)cc(-c3ncc4c(NCc5cc(C(=O)N(C)C)n(CC#N)n5)nc(OC[C@@]56CCCN5C[C@H](F)C6)nc4c3F)c12]

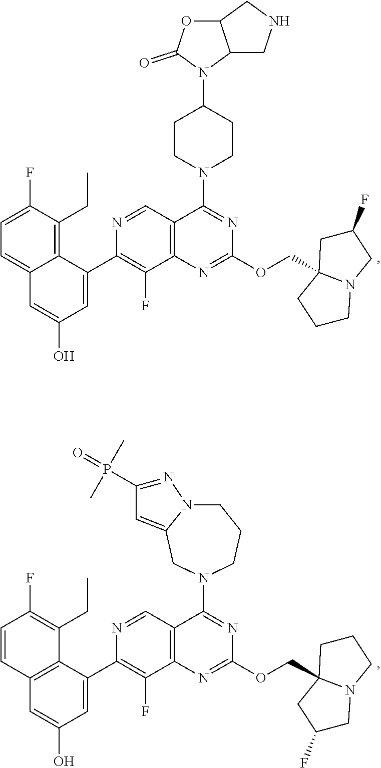 [SMILES: CCc1c(F)ccc2cc(O)cc(-c3ncc4c(N5CCC(N6C(=O)OC7CNCC76)CC5)nc(OC[C@@]56CCCN5C[C@H](F)C6)nc4c3F)c12 ; CCc1c(F)ccc2cc(O)cc(-c3ncc4c(N5CCCn6nc(P(C)(C)=O)cc6C5)nc(OC[C@@]56CCCN5C[C@H](F)C6)nc4c3F)c12]

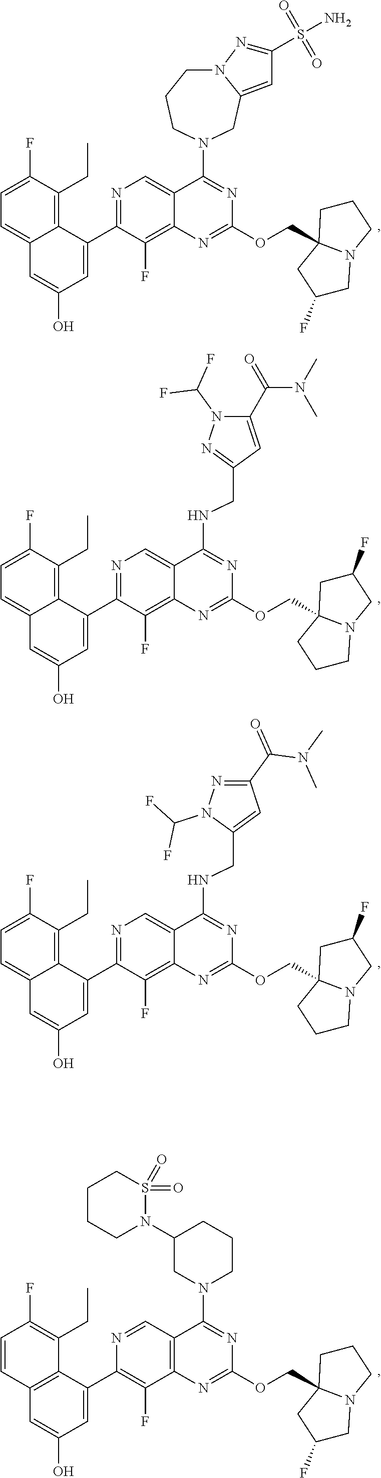

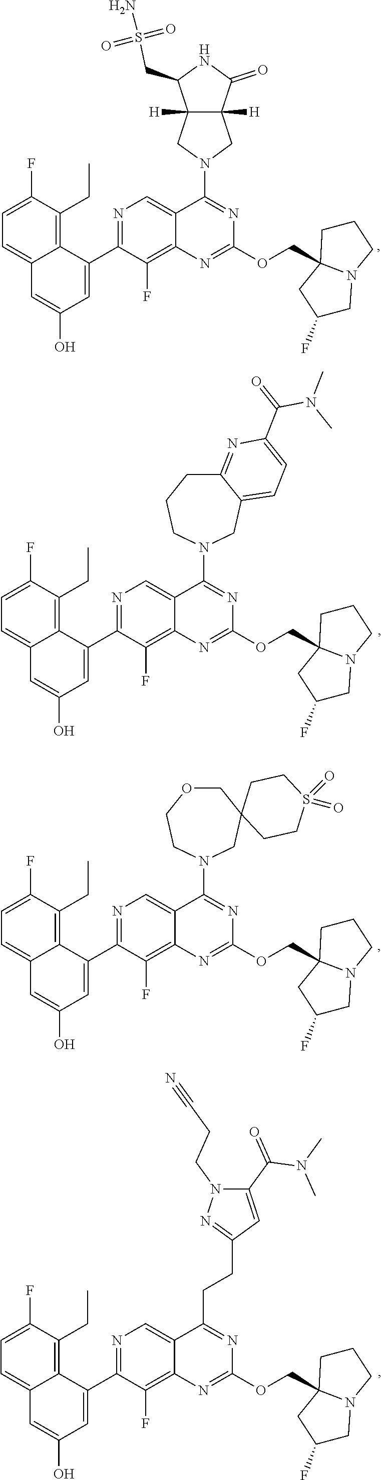

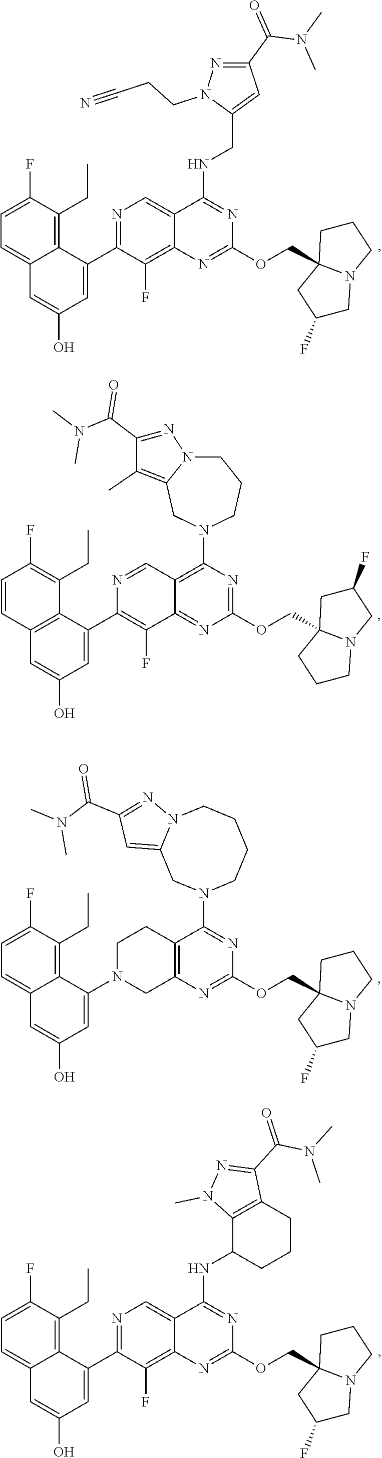

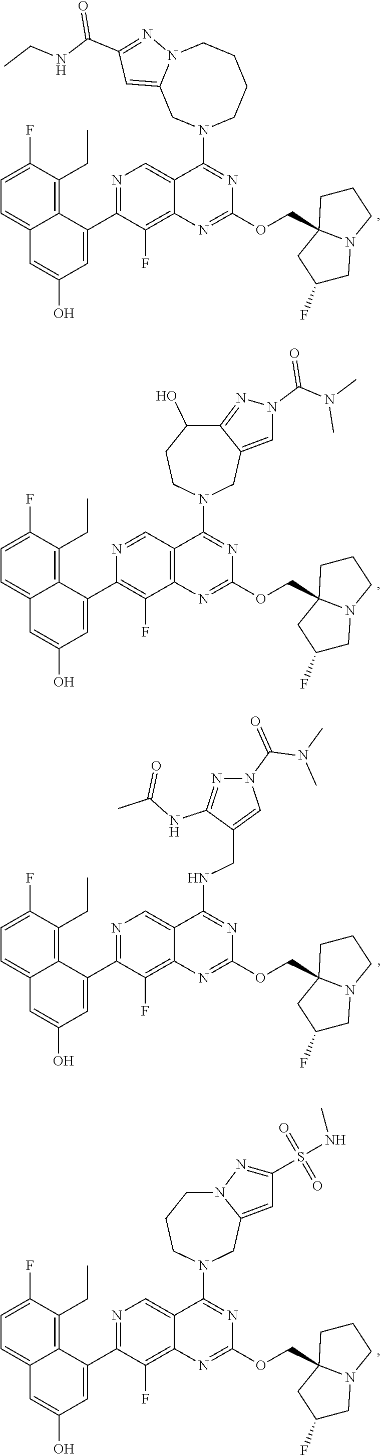

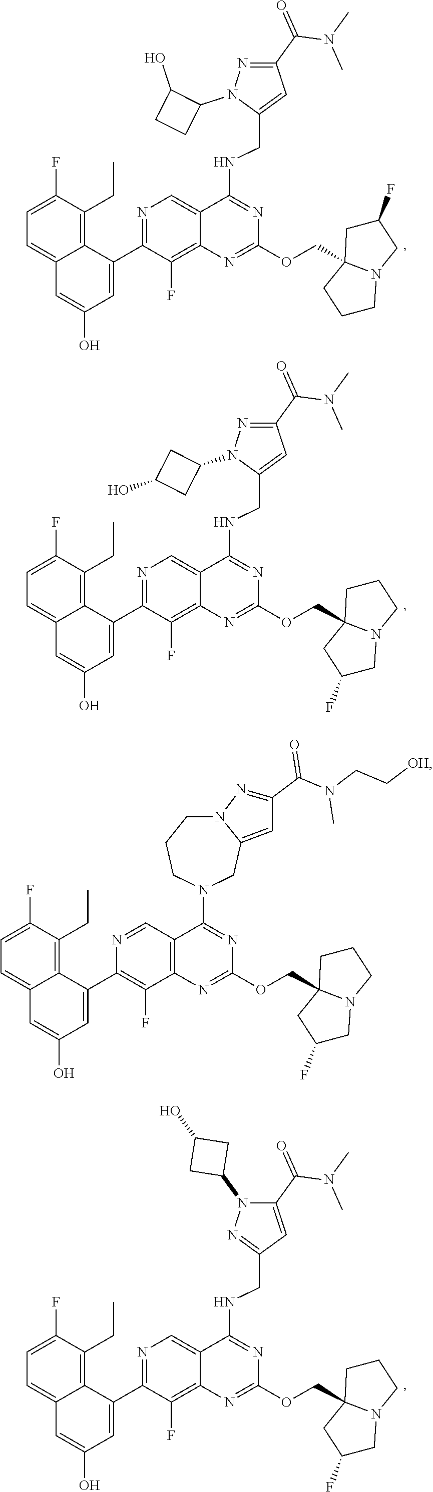

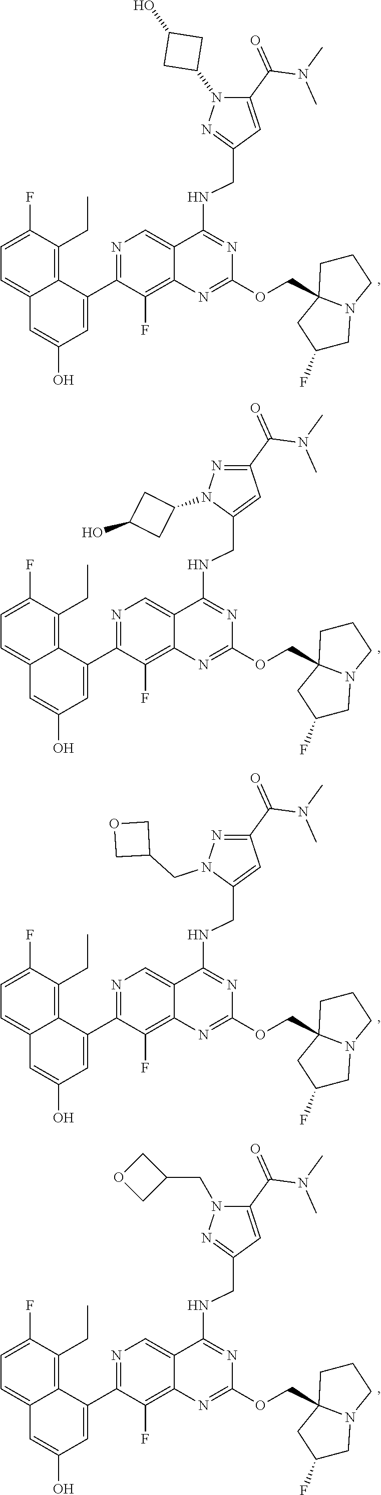

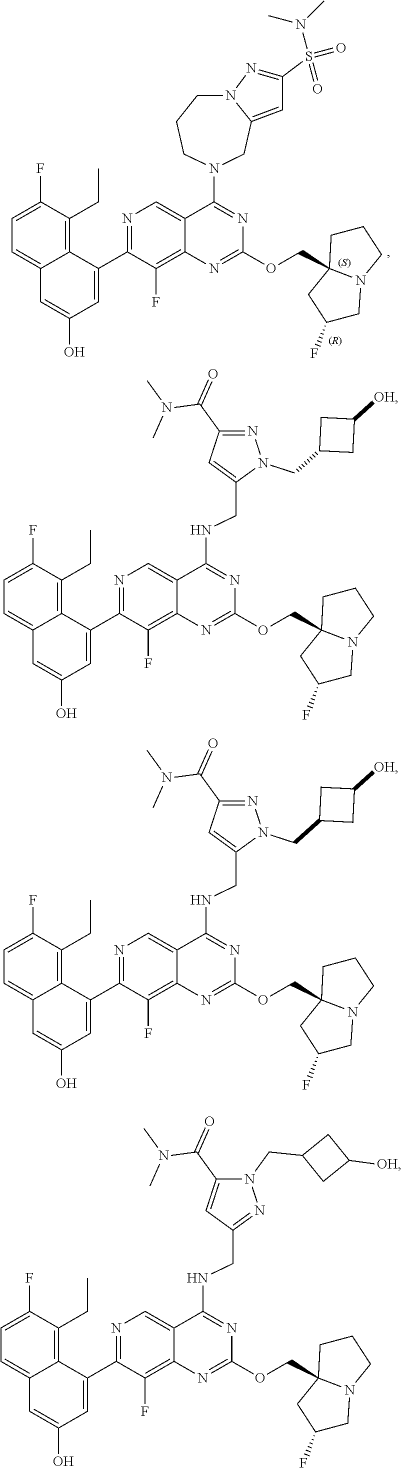

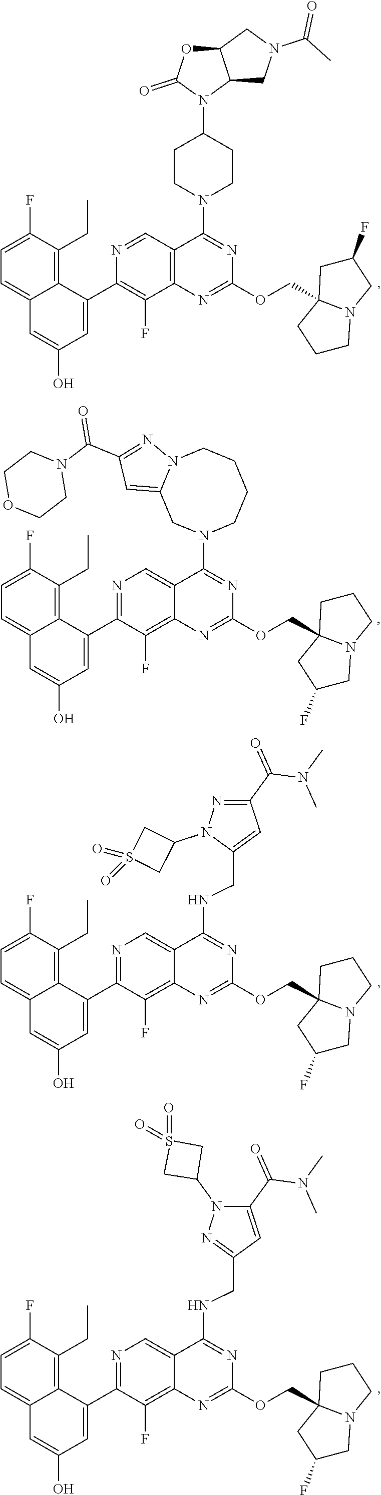

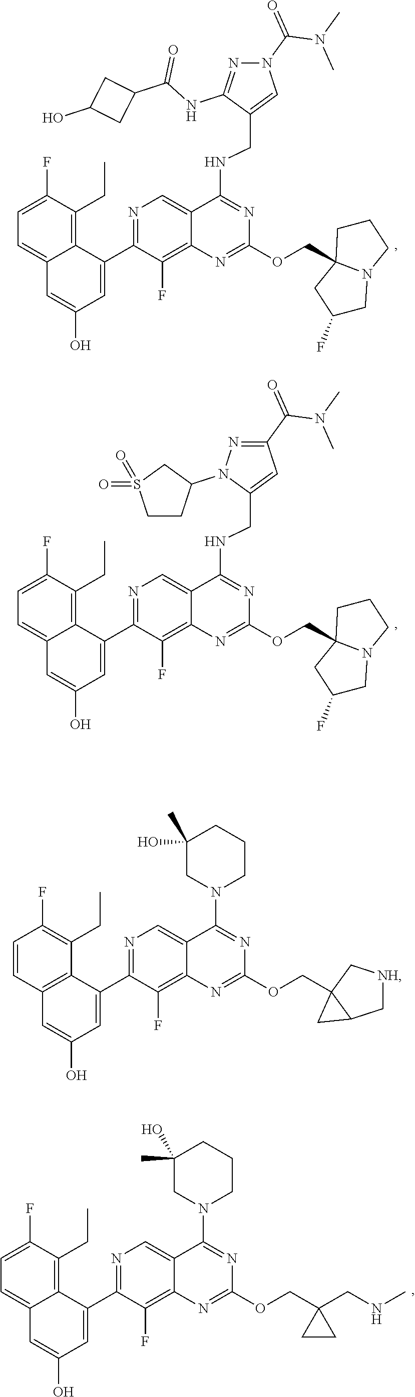 [SMILES: CCc1c(F)ccc2cc(O)cc(-c3ncc4c(NCc5cn(C(=O)N(C)C)nc5NC(=O)C5CC(O)C5)nc(OC[C@@]56CCCN5C[C@H](F)C6)nc4c3F)c12 ; CCc1c(F)ccc2cc(O)cc(-c3ncc4c(NCc5cc(C(=O)N(C)C)nn5C5CCS(=O)(=O)C5)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OCC56CNCC5C6)nc4c3F)c12 ; CCc1c(F)ccc2cc(O)cc(-c3ncc4c(N5CCC[C@@](C)(O)C5)nc(OCC5(CNC)CC5)nc4c3F)c12]

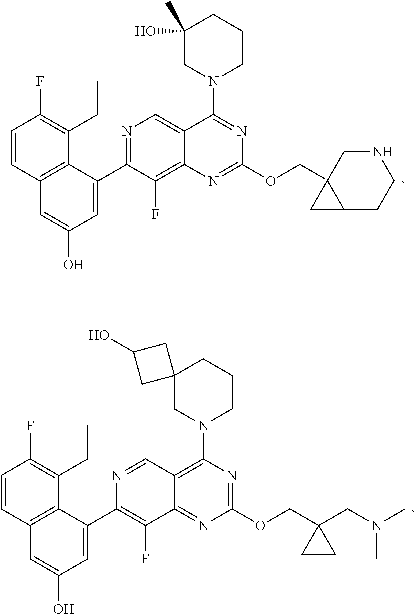

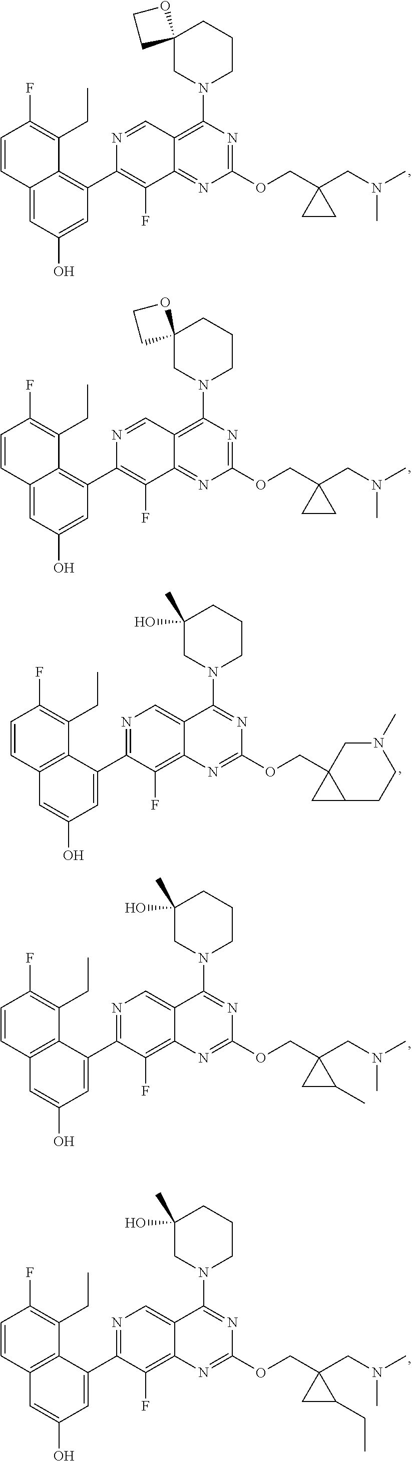 [SMILES: CCc1c(F)ccc2cc(O)cc(-c3ncc4c(N5CCC[C@]6(CCO6)C5)nc(OCC5(CN(C)C)CC5)nc4c3F)c12 ; CCc1c(F)ccc2cc(O)cc(-c3ncc4c(N5CCC[C@@]6(CCO6)C5)nc(OCC5(CN(C)C)CC5)nc4c3F)c12 ; CCc1c(F)ccc2cc(O)cc(-c3ncc4c(N5CCC[C@@](C)(O)C5)nc(OCC56CC5CCN(C)C6)nc4c3F)c12 ; CCc1c(F)ccc2cc(O)cc(-c3ncc4c(N5CCC[C@@](C)(O)C5)nc(OCC5(CN(C)C)CC5C)nc4c3F)c12 ; CCc1c(F)ccc2cc(O)cc(-c3ncc4c(N5CCC[C@@](C)(O)C5)nc(OCC5(CN(C)C)CC5CC)nc4c3F)c12]

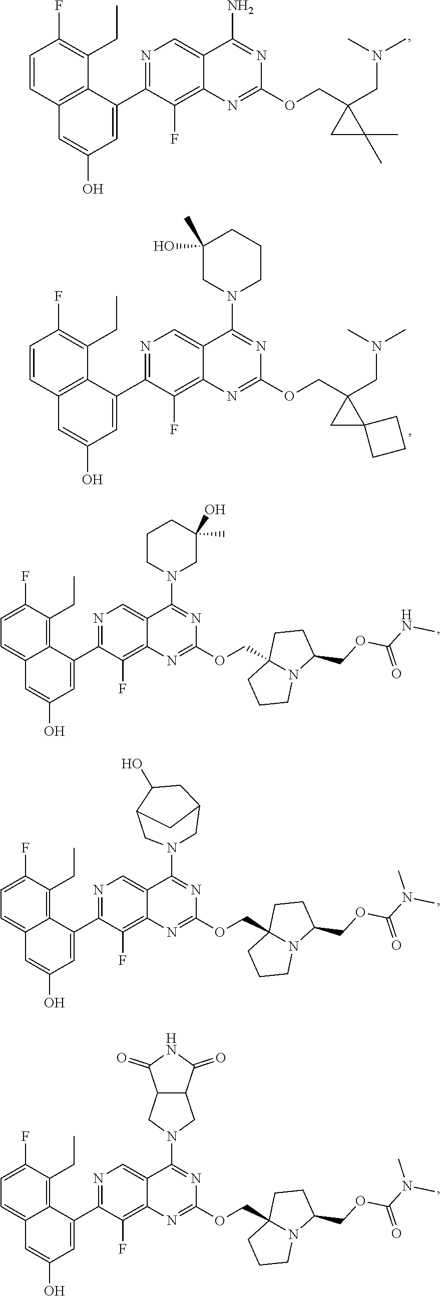 [SMILES: CCc1c(F)ccc2cc(O)cc(-c3ncc4c(N)nc(OCC5(CN(C)C)CC5(C)C)nc4c3F)c12 ; CCc1c(F)ccc2cc(O)cc(-c3ncc4c(N5CCC[C@@](C)(O)C5)nc(OCC5(CN(C)C)CC56CCC6)nc4c3F)c12 ; CCc1c(F)ccc2cc(O)cc(-c3ncc4c(N5CCC[C@@](C)(O)C5)nc(OC[C@@]56CCCN5[C@H](COC(=O)NC)CC6)nc4c3F)c12 ; CCc1c(F)ccc2cc(O)cc(-c3ncc4c(N5CC6CC(O)C(C6)C5)nc(OC[C@]56CCCN5[C@H](COC(=O)N(C)C)CC6)nc4c3F)c12 ; CCc1c(F)ccc2cc(O)cc(-c3ncc4c(N5CC6C(=O)NC(=O)C6C5)nc(OC[C@]56CCCN5[C@H](COC(=O)N(C)C)CC6)nc4c3F)c12]

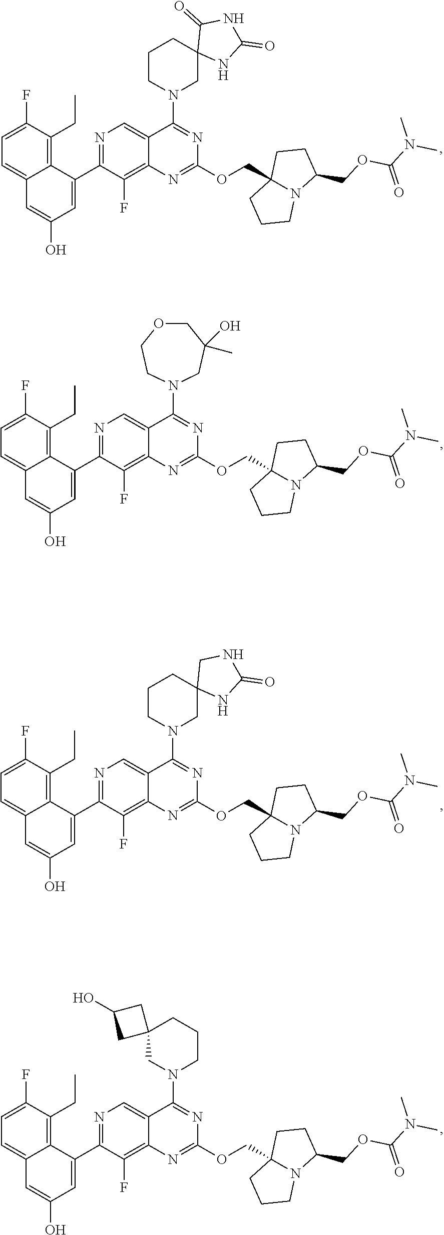 [SMILES: CCc1c(F)ccc2cc(O)cc(-c3ncc4c(N5CCCC6(C5)NC(=O)NC6=O)nc(OC[C@]56CCCN5[C@H](COC(=O)N(C)C)CC6)nc4c3F)c12 ; CCc1c(F)ccc2cc(O)cc(-c3ncc4c(N5CCOCC(C)(O)C5)nc(OC[C@@]56CCCN5[C@H](COC(=O)N(C)C)CC6)nc4c3F)c12 ; CCc1c(F)ccc2cc(O)cc(-c3ncc4c(N5CCCC6(CNC(=O)N6)C5)nc(OC[C@]56CCCN5[C@H](COC(=O)N(C)C)CC6)nc4c3F)c12 ; CCc1c(F)ccc2cc(O)cc(-c3ncc4c(N5CCC[C@]6(C5)C[C@H](O)C6)nc(OC[C@@]56CCCN5[C@H](COC(=O)N(C)C)CC6)nc4c3F)c12]

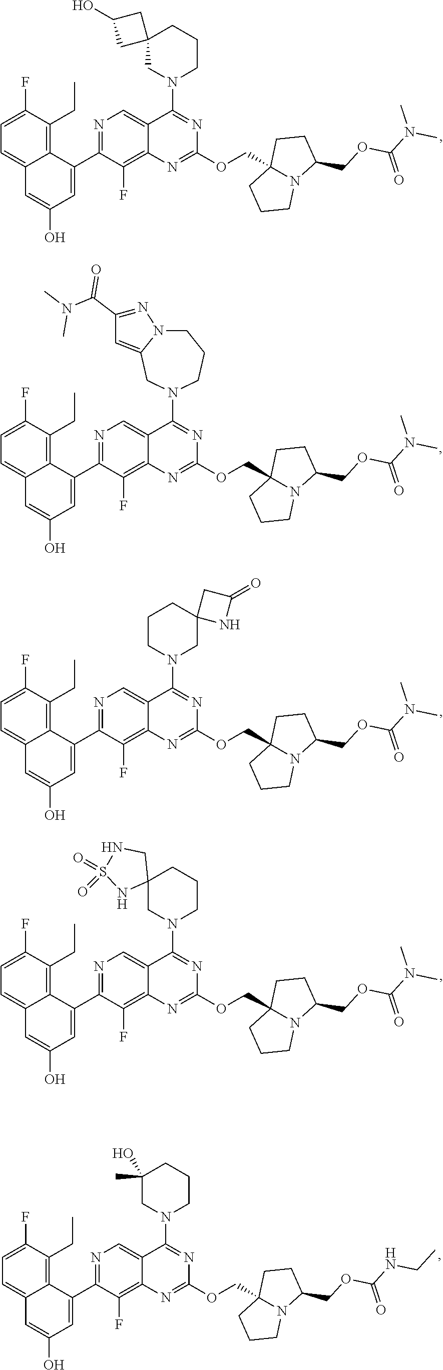

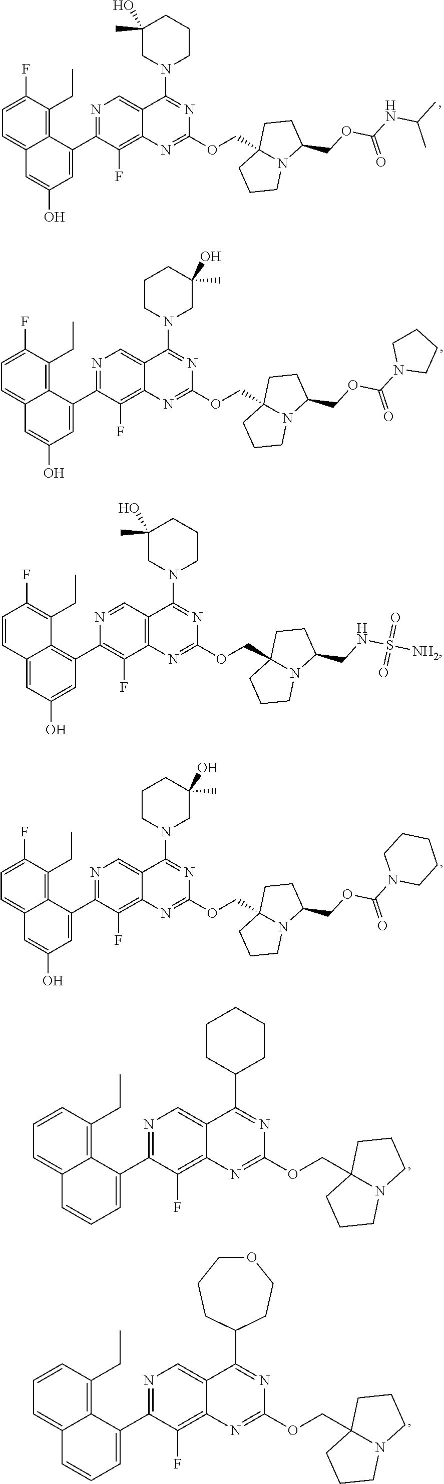

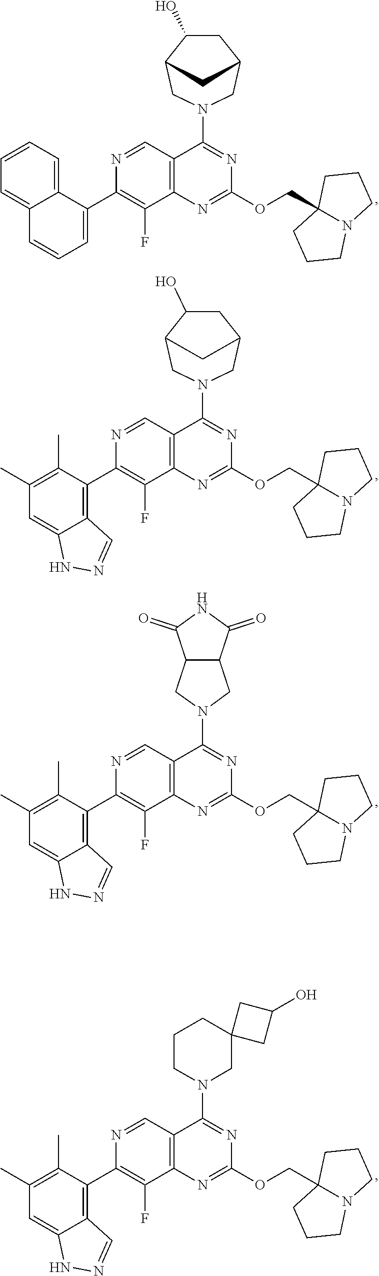

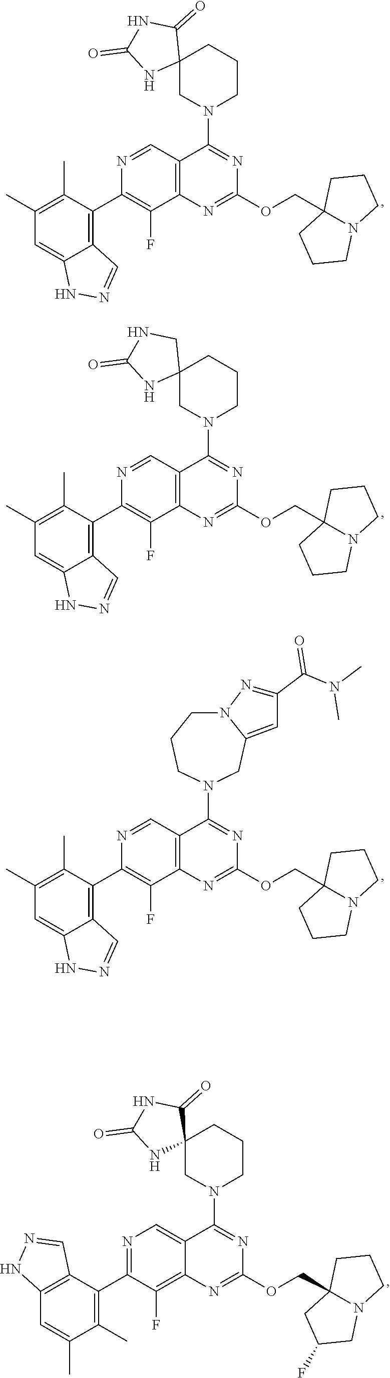 [SMILES: Cc1cc2[nH]ncc2c(-c2ncc3c(N4CCCC5(C4)NC(=O)NC5=O)nc(OCC45CCCN4CCC5)nc3c2F)c1C ; Cc1cc2[nH]ncc2c(-c2ncc3c(N4CCCC5(CNC(=O)N5)C4)nc(OCC45CCCN4CCC5)nc3c2F)c1C ; Cc1cc2[nH]ncc2c(-c2ncc3c(N4CCCn5nc(C(=O)N(C)C)cc5C4)nc(OCC45CCCN4CCC5)nc3c2F)c1C ; Cc1cc2[nH]ncc2c(-c2ncc3c(N4CCC[C@]5(C4)NC(=O)NC5=O)nc(OC[C@@]45CCCN4C[C@H](F)C5)nc3c2F)c1C]

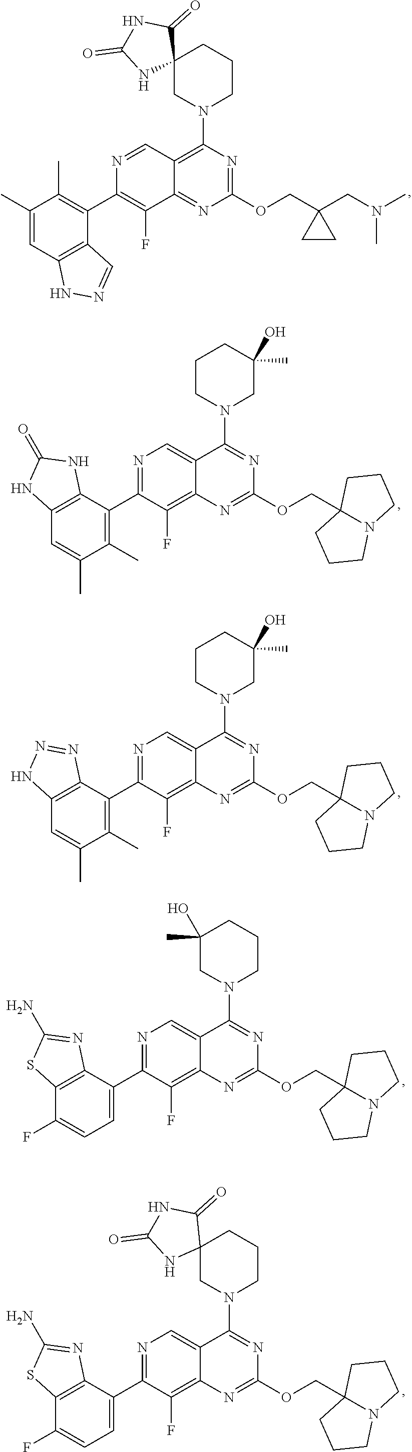

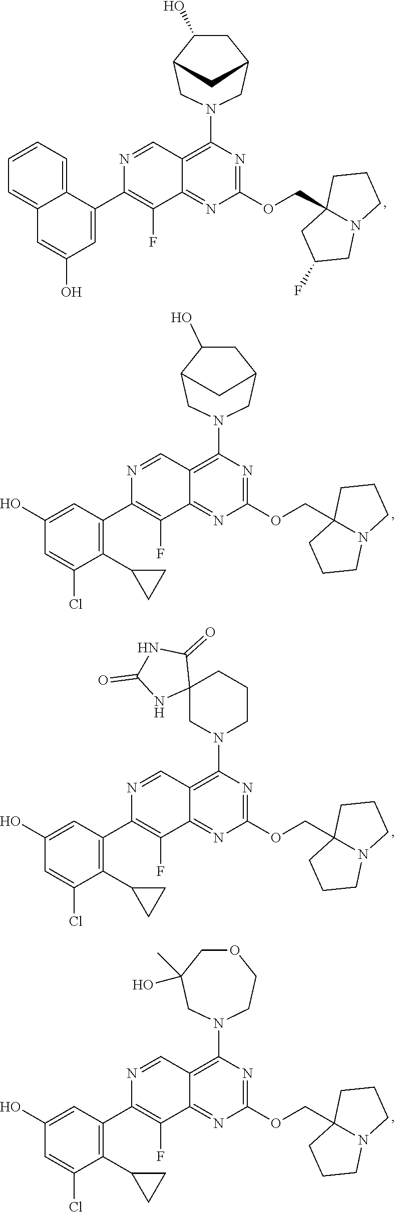

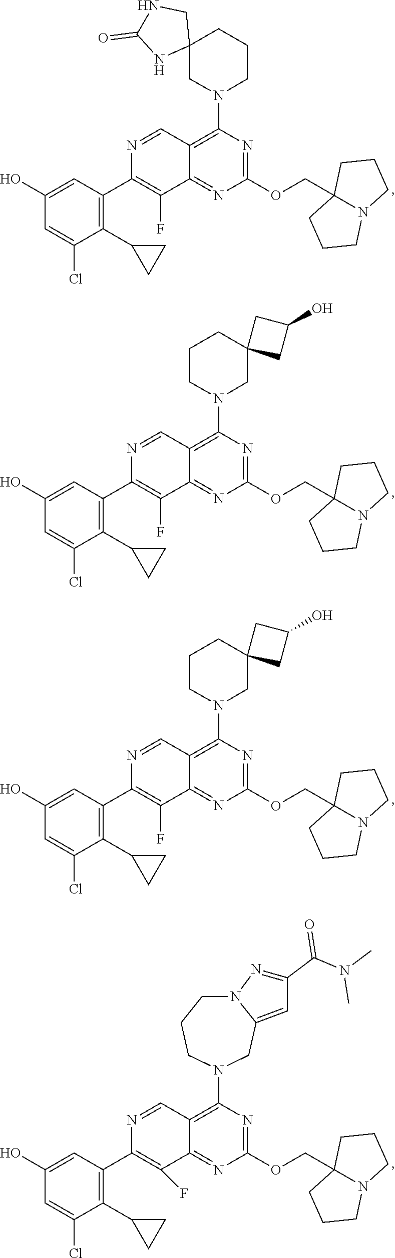 [SMILES: O=C1NCC2(CCCN(c3nc(OCC45CCCN4CCC5)nc4c(F)c(-c5cc(O)cc(Cl)c5C5CC5)ncc34)C2)N1 ; Oc1cc(Cl)c(C2CC2)c(-c2ncc3c(N4CCC[C@]5(C4)C[C@H](O)C5)nc(OCC45CCCN4CCC5)nc3c2F)c1 ; Oc1cc(Cl)c(C2CC2)c(-c2ncc3c(N4CCC[C@]5(C4)C[C@@H](O)C5)nc(OCC45CCCN4CCC5)nc3c2F)c1 ; CN(C)C(=O)c1cc2n(n1)CCCN(c1nc(OCC34CCCN3CCC4)nc3c(F)c(-c4cc(O)cc(Cl)c4C4CC4)ncc13)C2]

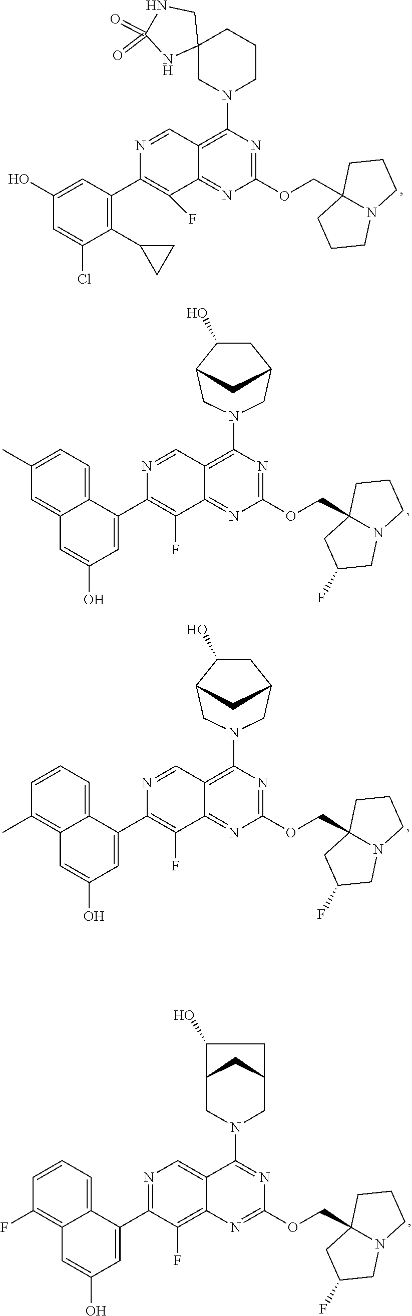

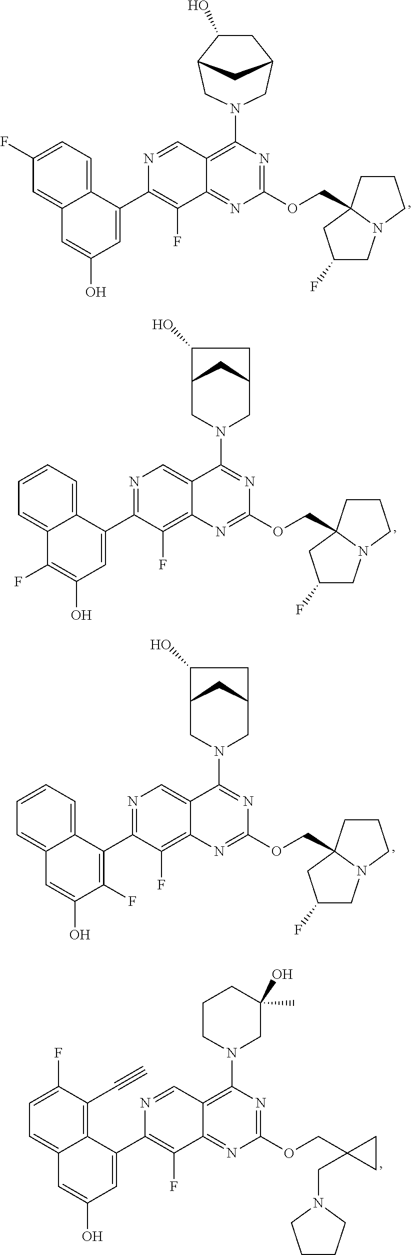 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccc(F)cc2c1 ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1F ; Oc1cc2ccccc2c(-c2ncc3c(N4C[C@@H]5C[C@H](C4)[C@H](O)C5)nc(OC[C@@]45CCCN4C[C@H](F)C5)nc3c2F)c1F ; C#Cc1c(F)ccc2cc(O)cc(-c3ncc4c(N5CCC[C@@](C)(O)C5)nc(OCC5(CN6CCCC6)CC5)nc4c3F)c12]

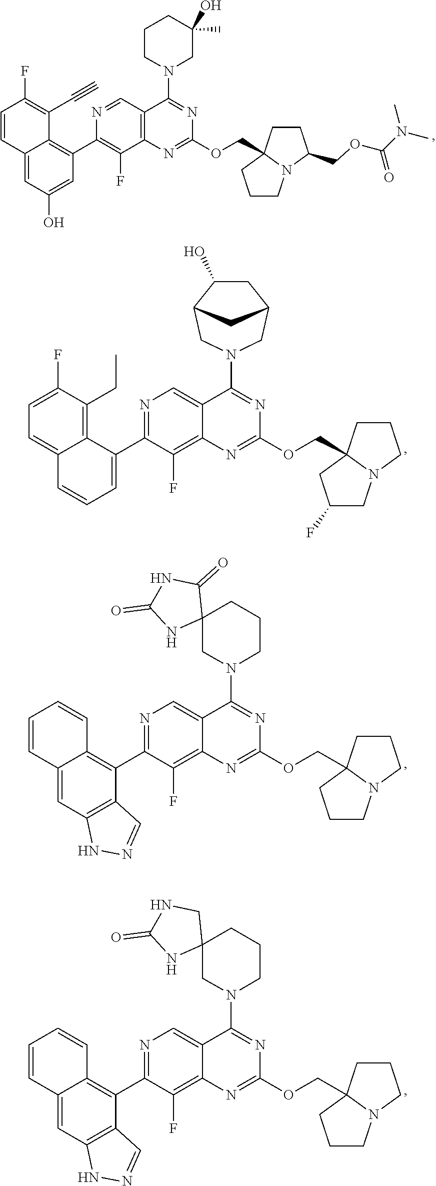 [SMILES: C#Cc1c(F)ccc2cc(O)cc(-c3ncc4c(N5CCC[C@@](C)(O)C5)nc(OC[C@]56CCCN5[C@H](COC(=O)N(C)C)CC6)nc4c3F)c12 ; CCc1c(F)ccc2cccc(-c3ncc4c(N5C[C@@H]6C[C@H](C5)[C@H](O)C6)nc(OC[C@@]56CCCN5C[C@H](F)C6)nc4c3F)c12 ; O=C1NC(=O)C2(CCCN(c3nc(OCC45CCCN4CCC5)nc4c(F)c(-c5c6ccccc6cc6[nH]ncc56)ncc34)C2)N1 ; O=C1NCC2(CCCN(c3nc(OCC45CCCN4CCC5)nc4c(F)c(-c5c6ccccc6cc6[nH]ncc56)ncc34)C2)N1]

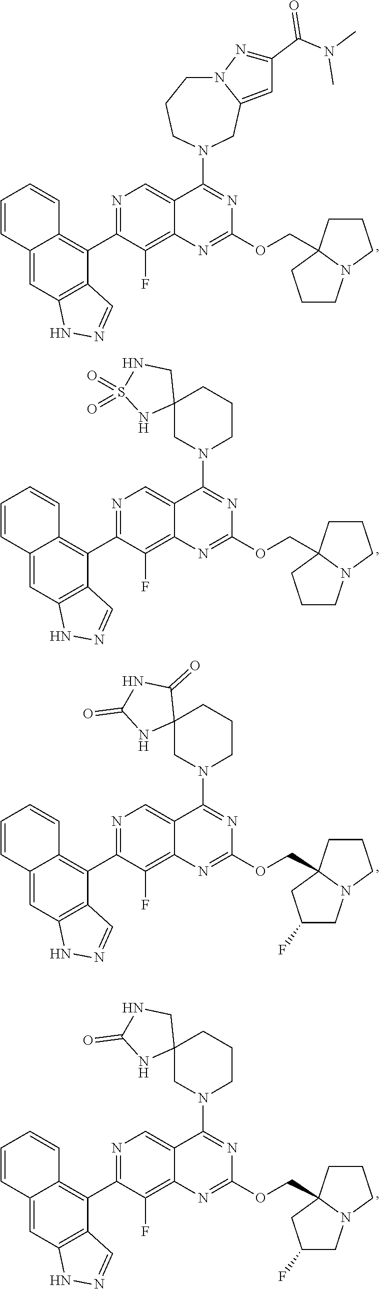

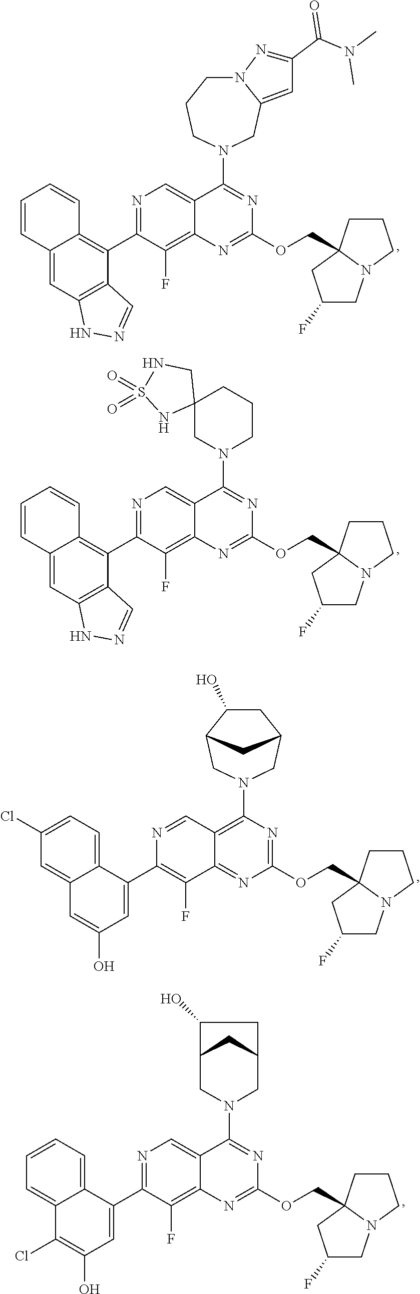

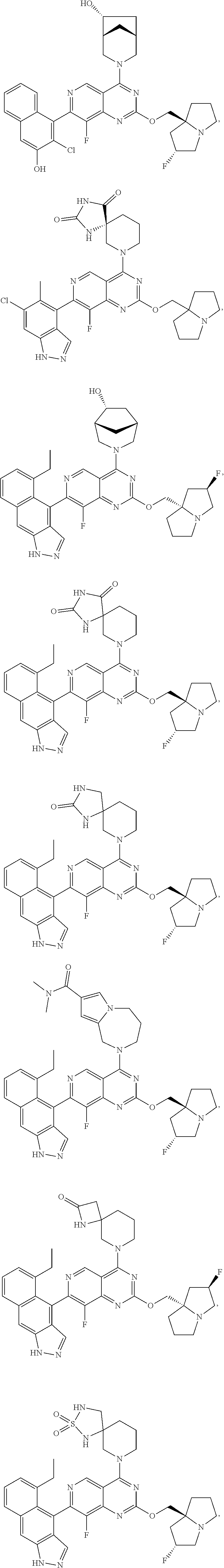

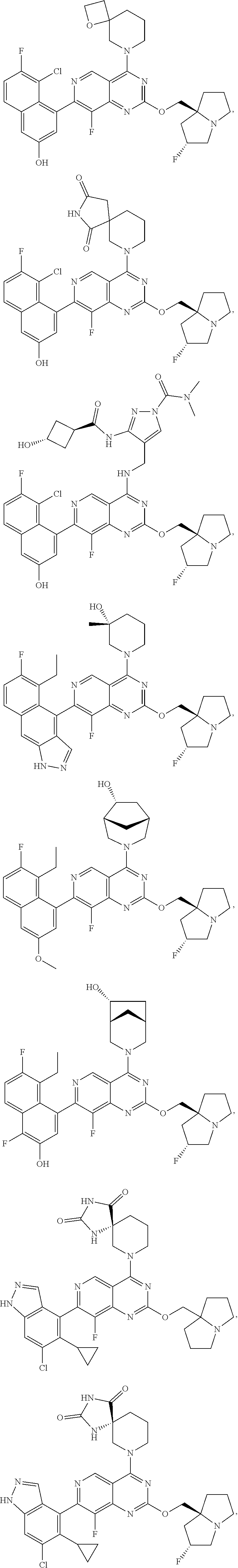

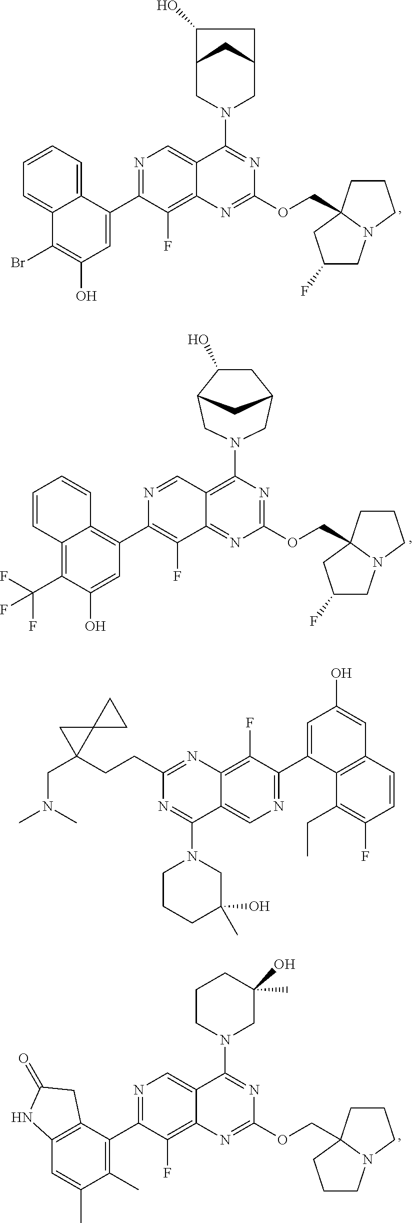

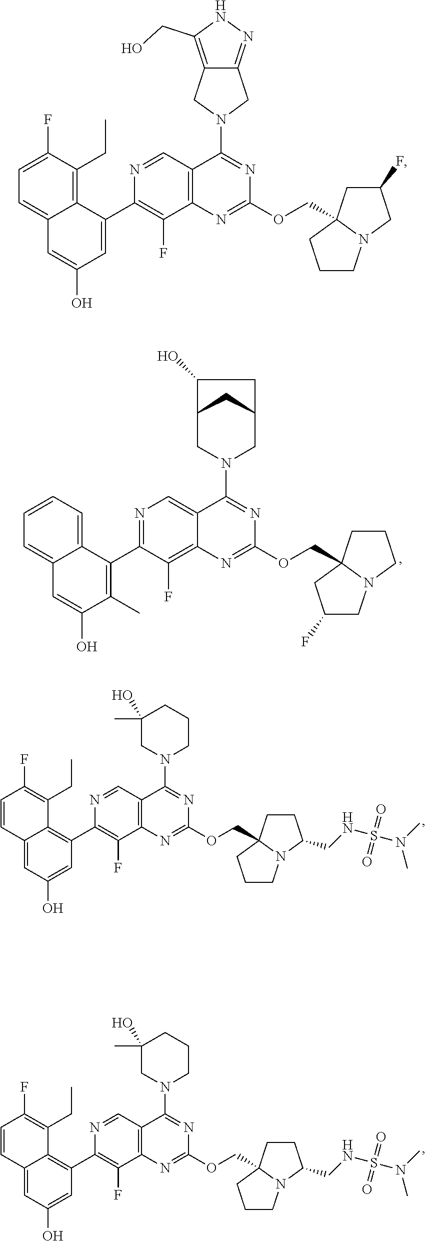 [SMILES: CCc1c(F)ccc2cc(O)cc(-c3ncc4c(N5Cc6n[nH]c(CO)c6C5)nc(OC[C@@]56CCCN5C[C@H](F)C6)nc4c3F)c12 ; Cc1c(O)cc2ccccc2c1-c1ncc2c(N3C[C@@H]4C[C@H](C3)[C@H](O)C4)nc(OC[C@@]34CCCN3C[C@H](F)C4)nc2c1F ; CCc1c(F)ccc2cc(O)cc(-c3ncc4c(N5CCC[C@@](C)(O)C5)nc(OC[C@]56CCCN5[C@@H](CNS(=O)(=O)N(C)C)CC6)nc4c3F)c12 ; CCc1c(F)ccc2cc(O)cc(-c3ncc4c(N5CCC[C@@](C)(O)C5)nc(OC[C@@]56CCCN5[C@@H](CNS(=O)(=O)N(C)C)CC6)nc4c3F)c12]

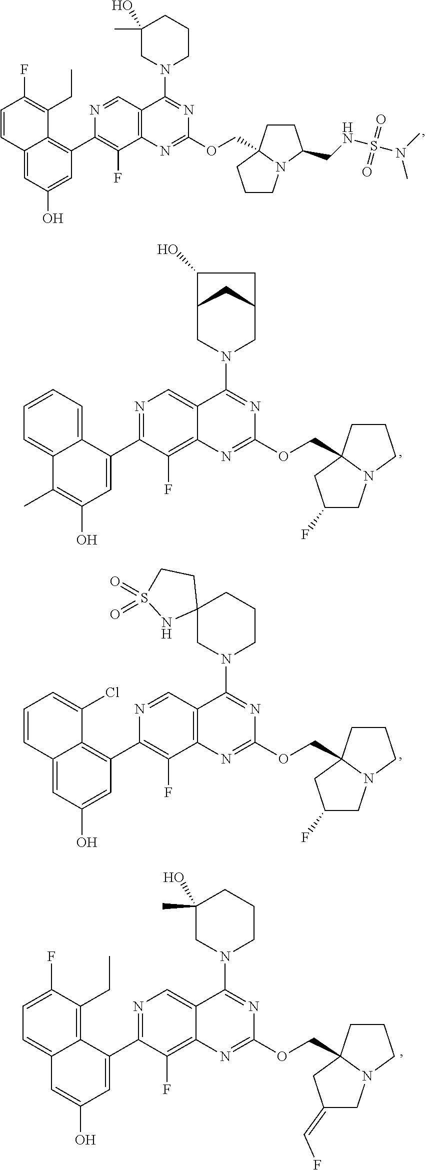 [SMILES: CCc1c(F)ccc2cc(O)cc(-c3ncc4c(N5CCC[C@@](C)(O)C5)nc(OC[C@@]56CCCN5[C@H](CNS(=O)(=O)N(C)C)CC6)nc4c3F)c12 ; Cc1c(O)cc(-c2ncc3c(N4C[C@@H]5C[C@H](C4)[C@H](O)C5)nc(OC[C@@]45CCCN4C[C@H](F)C5)nc3c2F)c2ccccc12 ; O=S1(=O)CCC2(CCCN(c3nc(OC[C@@]45CCCN4C[C@H](F)C5)nc4c(F)c(-c5cc(O)cc6cccc(Cl)c56)ncc34)C2)N1 ; CCc1c(F)ccc2cc(O)cc(-c3ncc4c(N5CCC[C@@](C)(O)C5)nc(OC[C@@]56CCCN5C/C(=C\F)C6)nc4c3F)c12]

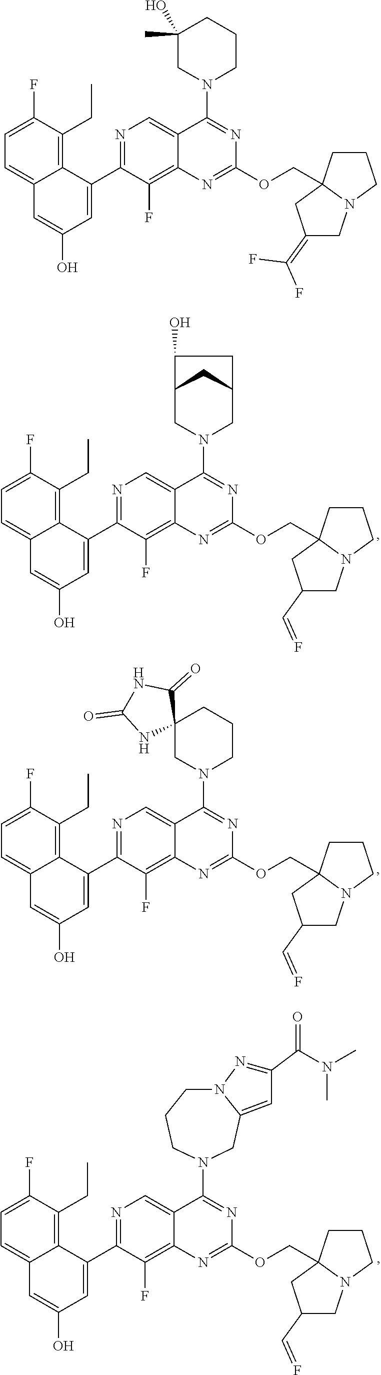

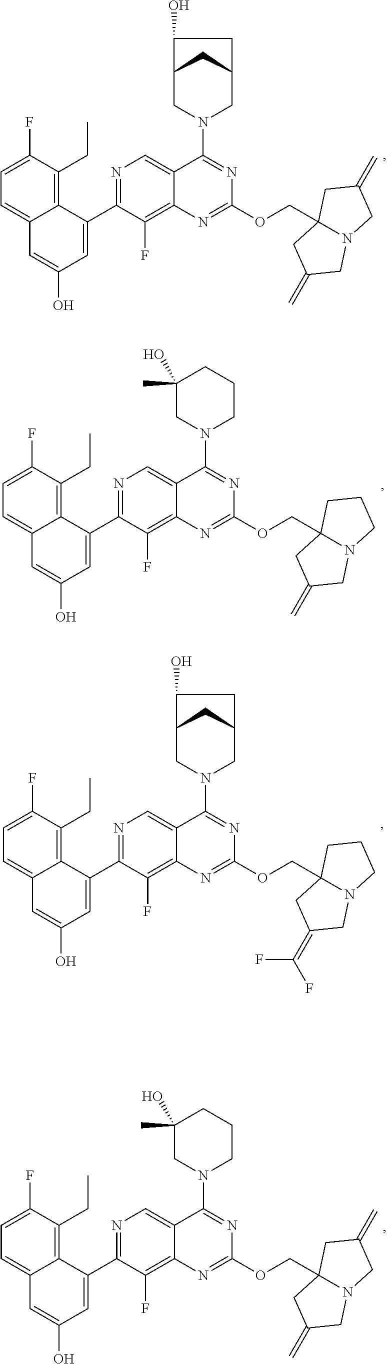 [SMILES: C=C1CN2CC(=C)CC2(COc2nc(N3C[C@@H]4C[C@H](C3)[C@H](O)C4)c3cnc(-c4cc(O)cc5ccc(F)c(CC)c45)c(F)c3n2)C1 ; C=C1CN2CCCC2(COc2nc(N3CCC[C@@](C)(O)C3)c3cnc(-c4cc(O)cc5ccc(F)c(CC)c45)c(F)c3n2)C1 ; CCc1c(F)ccc2cc(O)cc(-c3ncc4c(N5C[C@@H]6C[C@H](C5)[C@H](O)C6)nc(OCC56CCCN5CC(=C(F)F)C6)nc4c3F)c12 ; C=C1CN2CC(=C)CC2(COc2nc(N3CCC[C@@](C)(O)C3)c3cnc(-c4cc(O)cc5ccc(F)c(CC)c45)c(F)c3n2)C1]

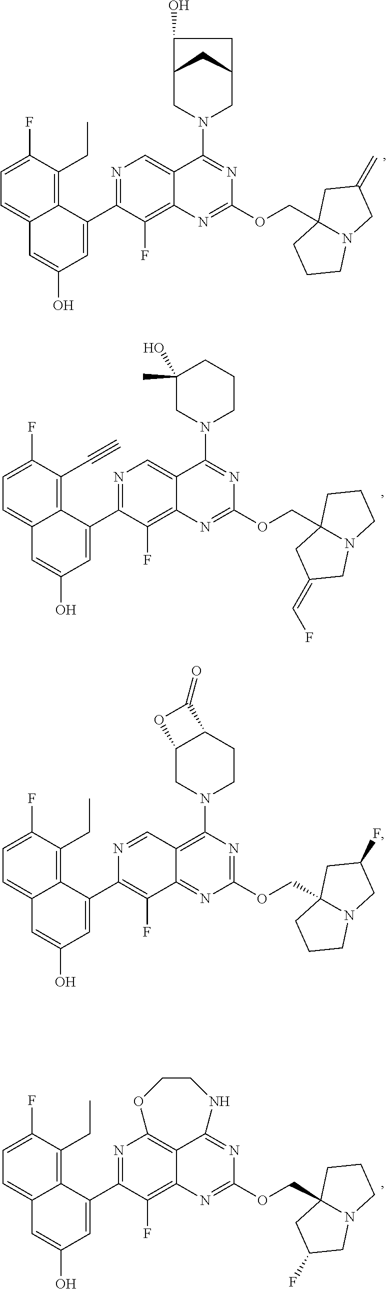 [SMILES: C=C1CN2CCCC2(COc2nc(N3C[C@@H]4C[C@H](C3)[C@H](O)C4)c3cnc(-c4cc(O)cc5ccc(F)c(CC)c45)c(F)c3n2)C1 ; C#Cc1c(F)ccc2cc(O)cc(-c3ncc4c(N5CCC[C@@](C)(O)C5)nc(OCC56CCCN5C/C(=C\F)C6)nc4c3F)c12 ; CCc1c(F)ccc2cc(O)cc(-c3ncc4c(N5CC[C@H]6C(=O)O[C@H]6C5)nc(OC[C@@]56CCCN5C[C@H](F)C6)nc4c3F)c12 ; CCc1c(F)ccc2cc(O)cc(-c3nc4c5c(nc(OC[C@@]67CCCN6C[C@H](F)C7)nc5c3F)NCCO4)c12]

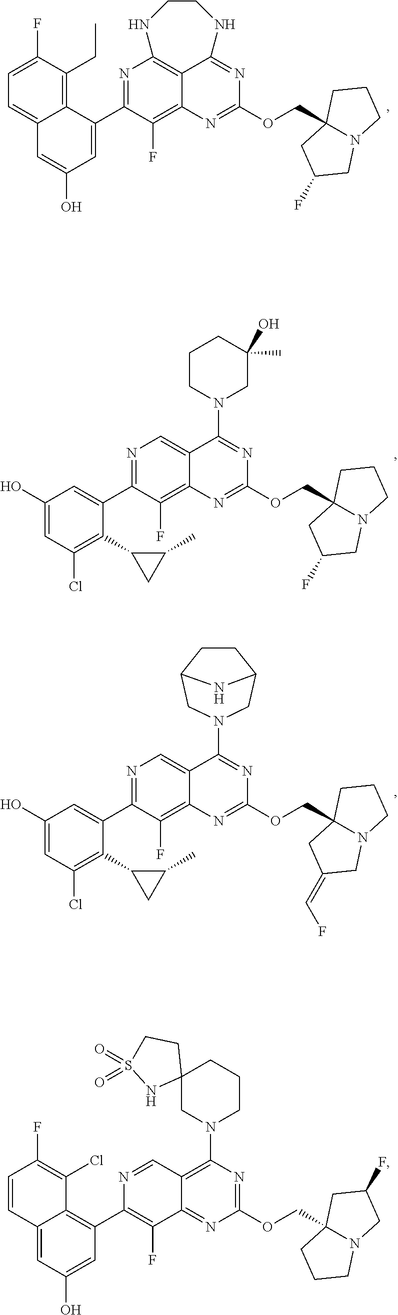 [SMILES: CCc1c(F)ccc2cc(O)cc(-c3nc4c5c(nc(OC[C@@]67CCCN6C[C@H](F)C7)nc5c3F)NCCN4)c12 ; C[C@@H]1C[C@@H]1c1c(Cl)cc(O)cc1-c1ncc2c(N3CCC[C@@](C)(O)C3)nc(OC[C@@]34CCCN3C[C@H](F)C4)nc2c1F ; C[C@@H]1C[C@@H]1c1c(Cl)cc(O)cc1-c1ncc2c(N3CC4CCC(C3)N4)nc(OC[C@@]34CCCN3C/C(=C\F)C4)nc2c1F ; O=S1(=O)CCC2(CCCN(c3nc(OC[C@@]45CCCN4C[C@H](F)C5)nc4c(F)c(-c5cc(O)cc6ccc(F)c(Cl)c56)ncc34)C2)N1]

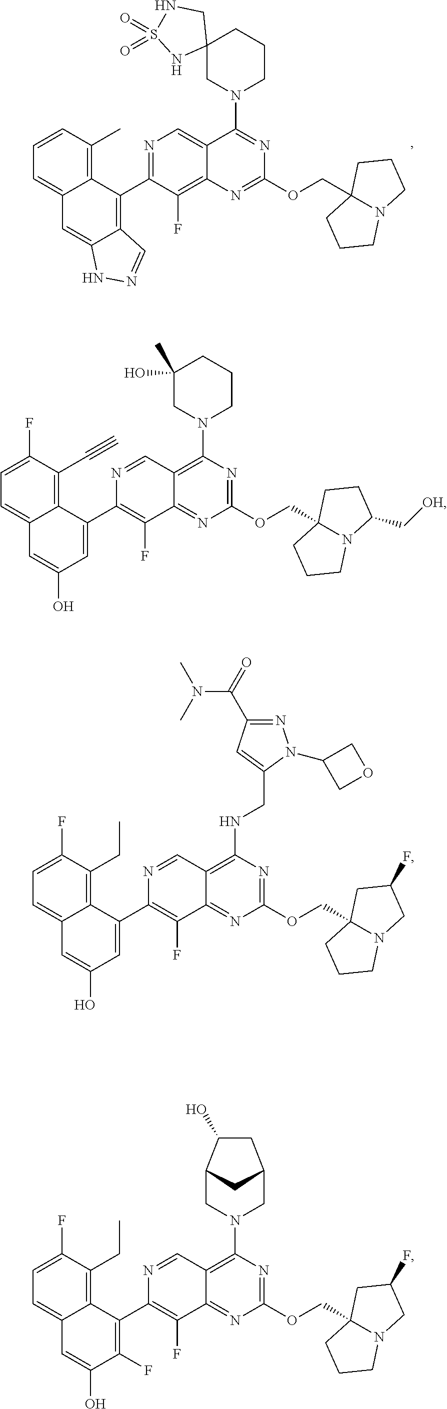 [SMILES: Cc1cccc2cc3[nH]ncc3c(-c3ncc4c(N5CCCC6(CNS(=O)(=O)N6)C5)nc(OCC56CCCN5CCC6)nc4c3F)c12 ; C#Cc1c(F)ccc2cc(O)cc(-c3ncc4c(N5CCC[C@@](C)(O)C5)nc(OC[C@@]56CCCN5[C@@H](CO)CC6)nc4c3F)c12 ; CCc1c(F)ccc2cc(O)cc(-c3ncc4c(NCc5cc(C(=O)N(C)C)nn5C5COC5)nc(OC[C@@]56CCCN5C[C@H](F)C6)nc4c3F)c12 ; CCc1c(F)ccc2cc(O)c(F)c(-c3ncc4c(N5C[C@@H]6C[C@H](C5)[C@H](O)C6)nc(OC[C@@]56CCCN5C[C@H](F)C6)nc4c3F)c12]

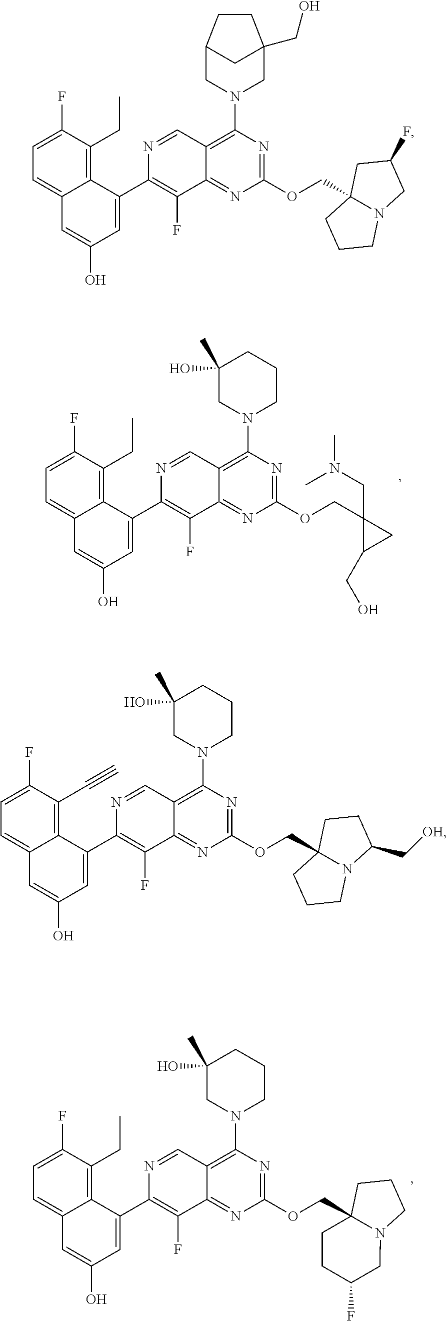 [SMILES: CCc1c(F)ccc2cc(O)cc(-c3ncc4c(N5CC6CCC(CO)(C6)C5)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OCC5(CN(C)C)CC5CO)nc4c3F)c12 ; C#Cc1c(F)ccc2cc(O)cc(-c3ncc4c(N5CCC[C@@](C)(O)C5)nc(OC[C@]56CCCN5[C@H](CO)CC6)nc4c3F)c12 ; CCc1c(F)ccc2cc(O)cc(-c3ncc4c(N5CCC[C@@](C)(O)C5)nc(OC[C@@]56CCCN5C[C@H](F)CC6)nc4c3F)c12]

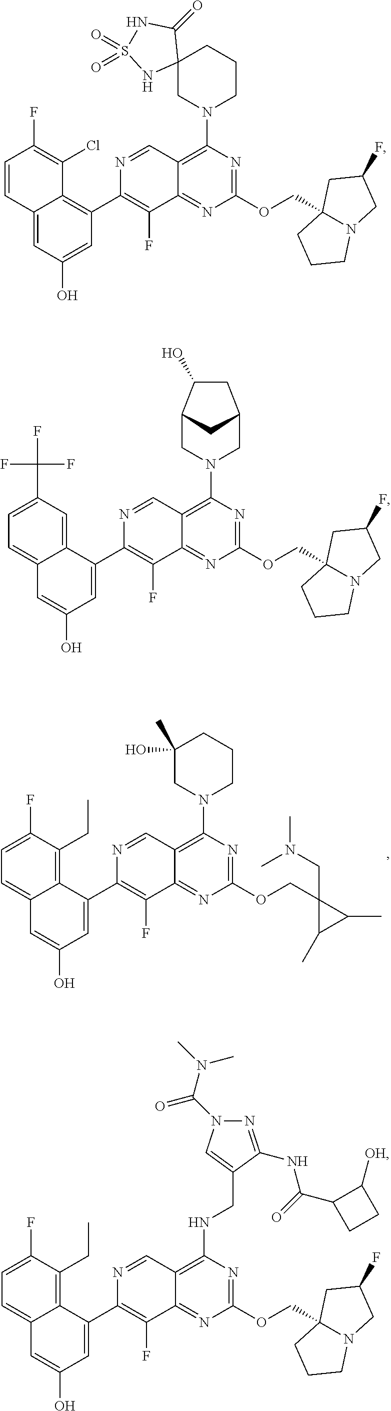 [SMILES: O=C1NS(=O)(=O)NC12CCCN(c1nc(OC[C@@]34CCCN3C[C@H](F)C4)nc3c(F)c(-c4cc(O)cc5ccc(F)c(Cl)c45)ncc13)C2 ; Oc1cc(-c2ncc3c(N4C[C@@H]5C[C@H](C4)[C@H](O)C5)nc(OC[C@@]45CCCN4C[C@H](F)C5)nc3c2F)c2cc(C(F)(F)F)ccc2c1 ; CCc1c(F)ccc2cc(O)cc(-c3ncc4c(N5CCC[C@@](C)(O)C5)nc(OCC5(CN(C)C)C(C)C5C)nc4c3F)c12 ; CCc1c(F)ccc2cc(O)cc(-c3ncc4c(NCc5cn(C(=O)N(C)C)nc5NC(=O)C5CCC5O)nc(OC[C@@]56CCCN5C[C@H](F)C6)nc4c3F)c12]

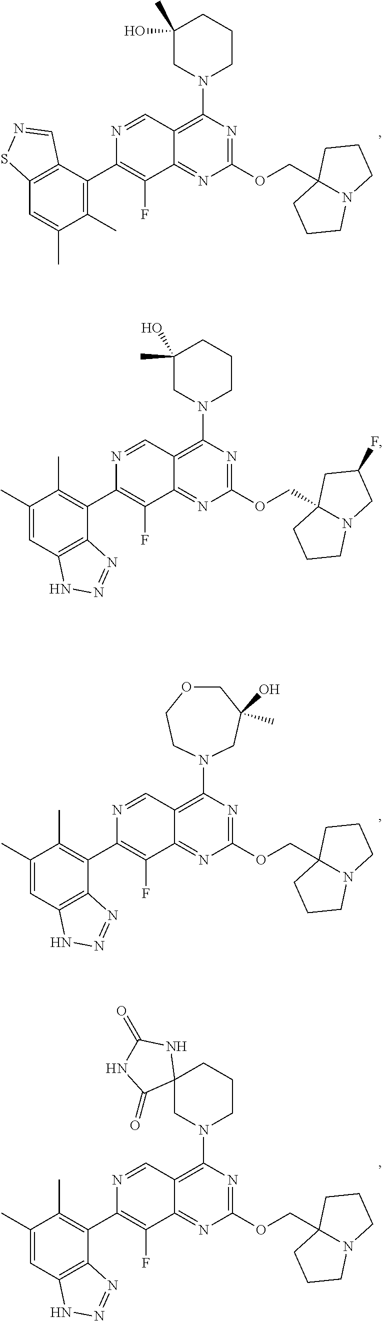 [SMILES: Cc1cc2sncc2c(-c2ncc3c(N4CCC[C@@](C)(O)C4)nc(OCC45CCCN4CCC5)nc3c2F)c1C ; Cc1cc2[nH]nnc2c(-c2ncc3c(N4CCC[C@@](C)(O)C4)nc(OC[C@@]45CCCN4C[C@H](F)C5)nc3c2F)c1C ; Cc1cc2[nH]nnc2c(-c2ncc3c(N4CCOC[C@@](C)(O)C4)nc(OCC45CCCN4CCC5)nc3c2F)c1C ; Cc1cc2[nH]nnc2c(-c2ncc3c(N4CCCC5(C4)NC(=O)NC5=O)nc(OCC45CCCN4CCC5)nc3c2F)c1C]

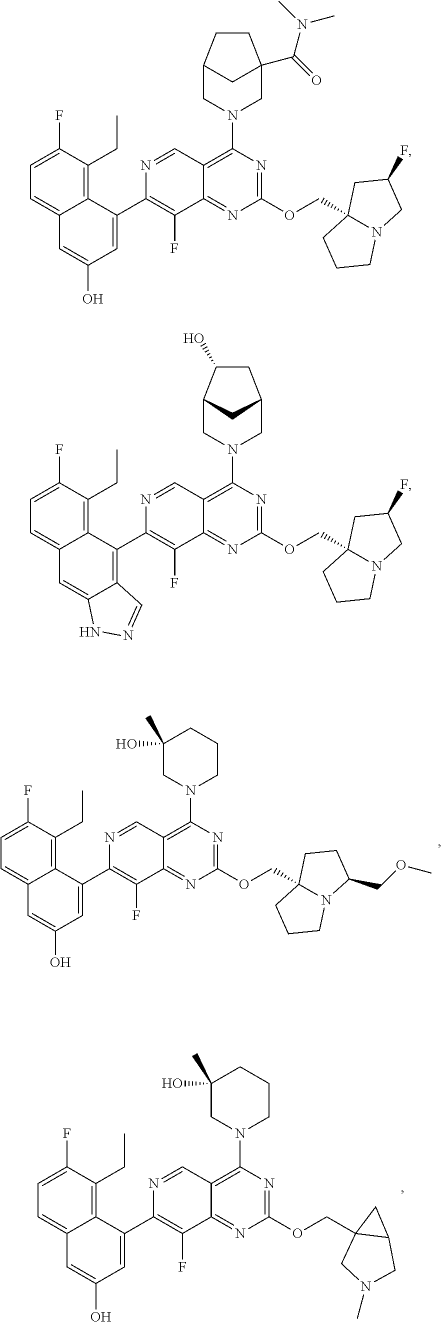 [SMILES: CCc1c(F)ccc2cc(O)cc(-c3ncc4c(N5CC6CCC(C(=O)N(C)C)(C6)C5)nc(OC[C@@]56CCCN5C[C@H](F)C6)nc4c3F)c12 ; CCc1c(F)ccc2cc3[nH]ncc3c(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OC[C@@]56CCCN5[C@H](COC)CC6)nc4c3F)c12 ; CCc1c(F)ccc2cc(O)cc(-c3ncc4c(N5CCC[C@@](C)(O)C5)nc(OCC56CC5CN(C)C6)nc4c3F)c12]

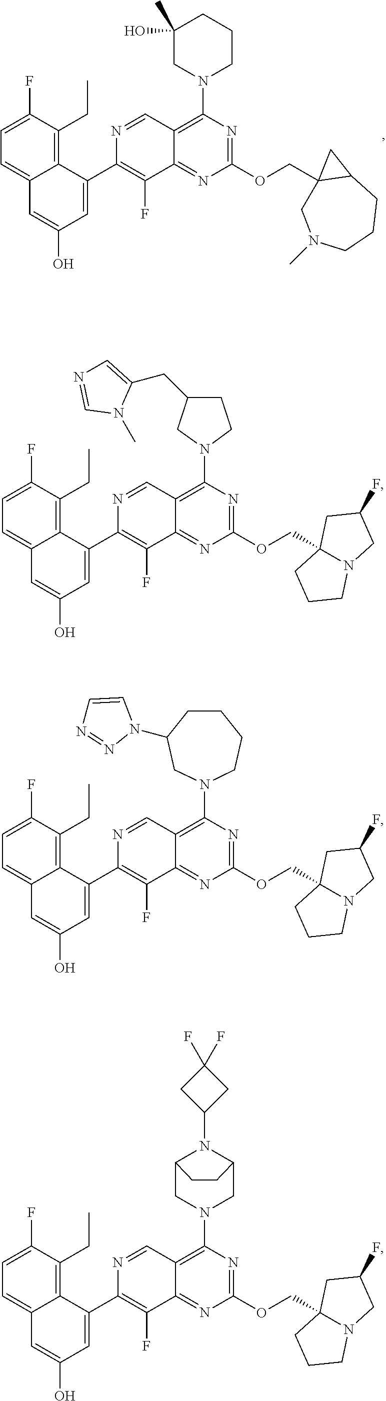 [SMILES: CCc1c(F)ccc2cc(O)cc(-c3ncc4c(N5CCC[C@@](C)(O)C5)nc(OCC56CC5CCCN(C)C6)nc4c3F)c12 ; CCc1c(F)ccc2cc(O)cc(-c3ncc4c(N5CCC(Cc6cncn6C)C5)nc(OC[C@@]56CCCN5C[C@H](F)C6)nc4c3F)c12 ; CCc1c(F)ccc2cc(O)cc(-c3ncc4c(N5CCCCC(n6ccnn6)C5)nc(OC[C@@]56CCCN5C[C@H](F)C6)nc4c3F)c12 ; CCc1c(F)ccc2cc(O)cc(-c3ncc4c(N5CC6CCC(C5)N6C5CC(F)(F)C5)nc(OC[C@@]56CCCN5C[C@H](F)C6)nc4c3F)c12]

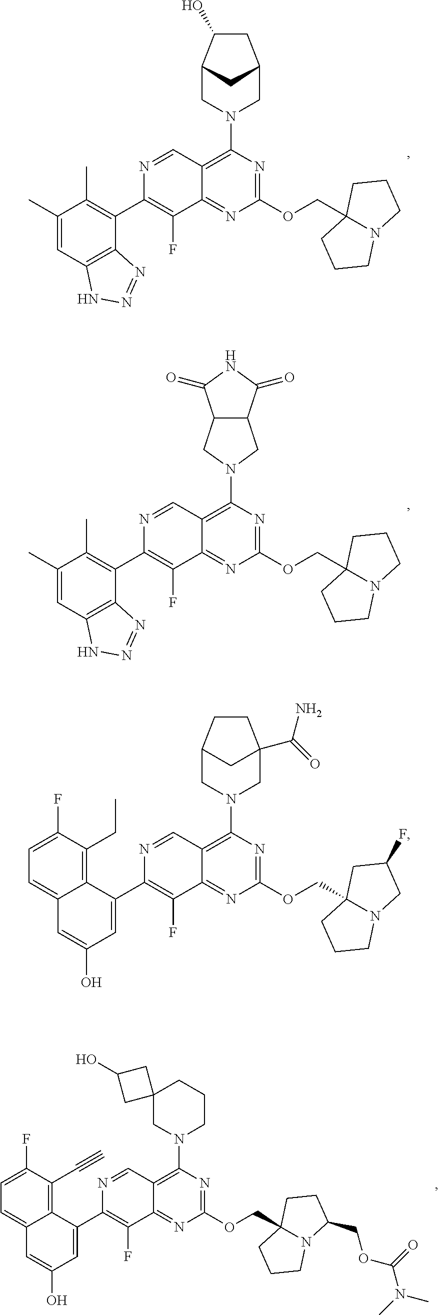 [SMILES: Cc1cc2[nH]nnc2c(-c2ncc3c(N4C[C@@H]5C[C@H](C4)[C@H](O)C5)nc(OCC45CCCN4CCC5)nc3c2F)c1C ; Cc1cc2[nH]nnc2c(-c2ncc3c(N4CC5C(=O)NC(=O)C5C4)nc(OCC45CCCN4CCC5)nc3c2F)c1C ; CCc1c(F)ccc2cc(O)cc(-c3ncc4c(N5CC6CCC(C(N)=O)(C6)C5)nc(OC[C@@]56CCCN5C[C@H](F)C6)nc4c3F)c12 ; C#Cc1c(F)ccc2cc(O)cc(-c3ncc4c(N5CCCC6(CC(O)C6)C5)nc(OC[C@]56CCCN5[C@H](COC(=O)N(C)C)CC6)nc4c3F)c12]

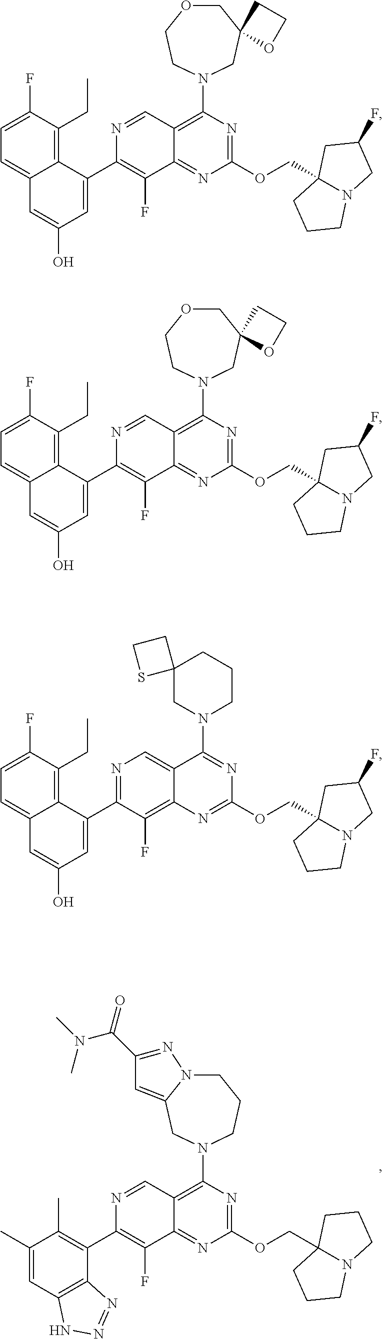 [SMILES: CCc1c(F)ccc2cc(O)cc(-c3ncc4c(N5CCOC[C@@]6(CCO6)C5)nc(OC[C@@]56CCCN5C[C@H](F)C6)nc4c3F)c12 ; CCc1c(F)ccc2cc(O)cc(-c3ncc4c(N5CCOC[C@]6(CCO6)C5)nc(OC[C@@]56CCCN5C[C@H](F)C6)nc4c3F)c12 ; CCc1c(F)ccc2cc(O)cc(-c3ncc4c(N5CCCC6(CCS6)C5)nc(OC[C@@]56CCCN5C[C@H](F)C6)nc4c3F)c12 ; Cc1cc2[nH]nnc2c(-c2ncc3c(N4CCCn5nc(C(=O)N(C)C)cc5C4)nc(OCC45CCCN4CCC5)nc3c2F)c1C]

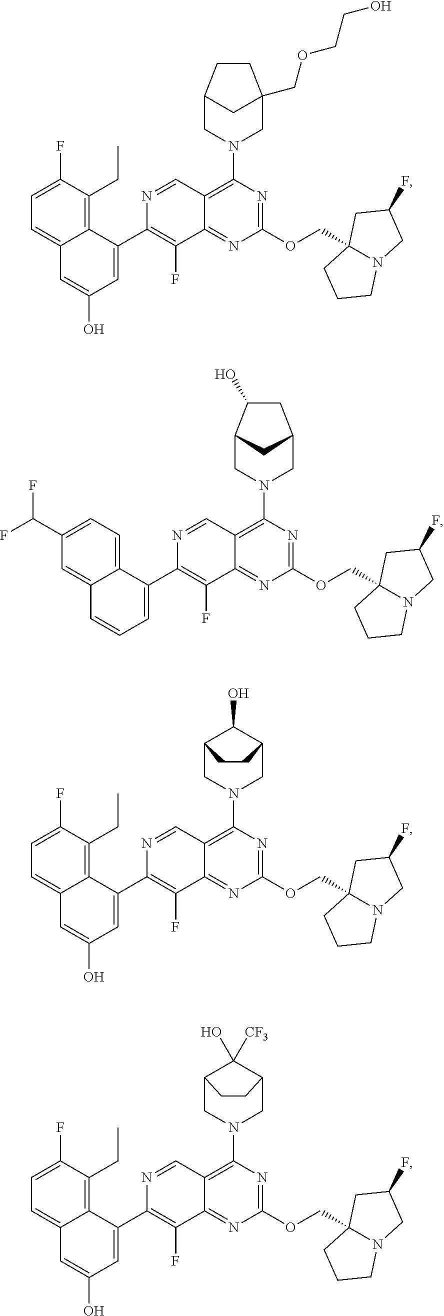 [SMILES: CCc1c(F)ccc2cc(O)cc(-c3ncc4c(N5CC6CCC(COCCO)(C6)C5)nc(OC[C@@]56CCCN5C[C@H](F)C6)nc4c3F)c12 ; O[C@@H]1C[C@H]2C[C@@H]1CN(c1nc(OC[C@@]34CCCN3C[C@H](F)C4)nc3c(F)c(-c4cccc5cc(C(F)F)ccc45)ncc13)C2 ; CCc1c(F)ccc2cc(O)cc(-c3ncc4c(N5C[C@H]6CC[C@@H](C5)[C@@H]6O)nc(OC[C@@]56CCCN5C[C@H](F)C6)nc4c3F)c12 ; CCc1c(F)ccc2cc(O)cc(-c3ncc4c(N5CC6CCC(C5)C6(O)C(F)(F)F)nc(OC[C@@]56CCCN5C[C@H](F)C6)nc4c3F)c12]

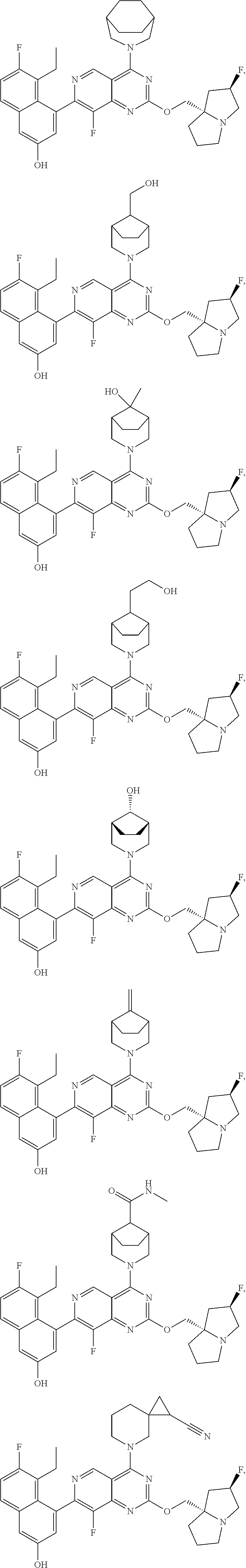

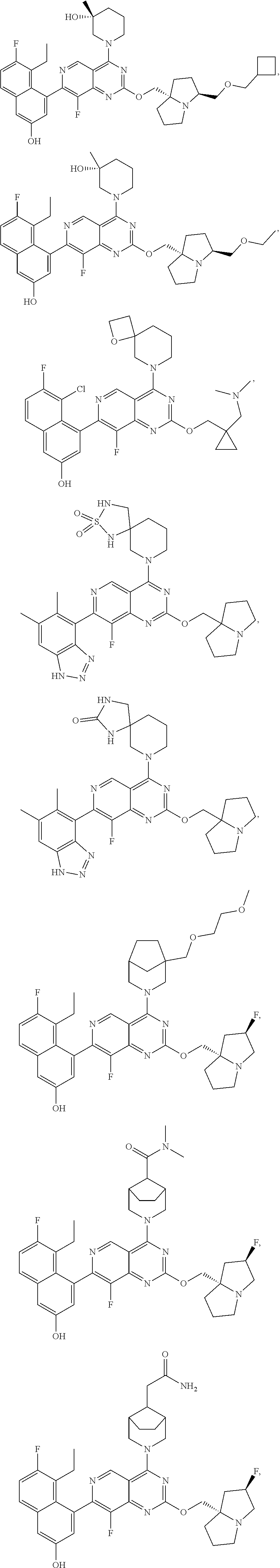

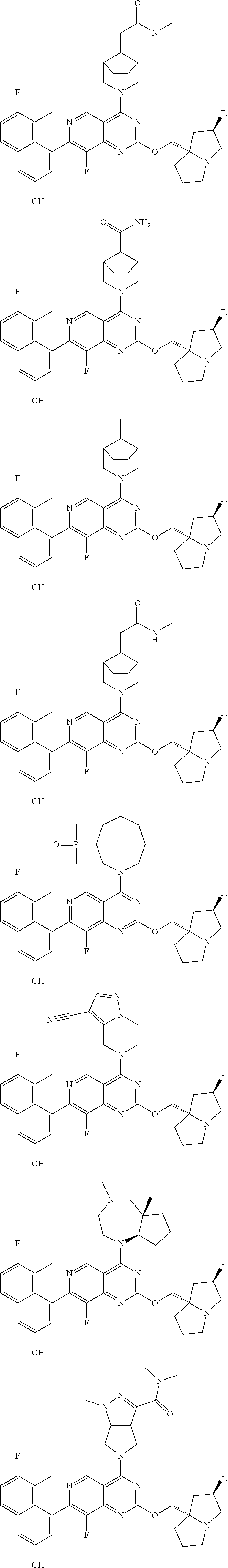

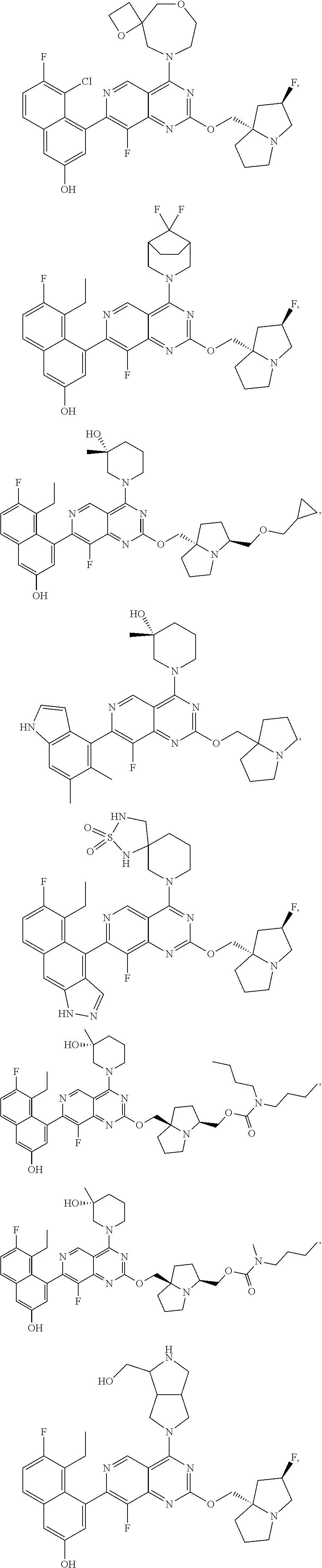

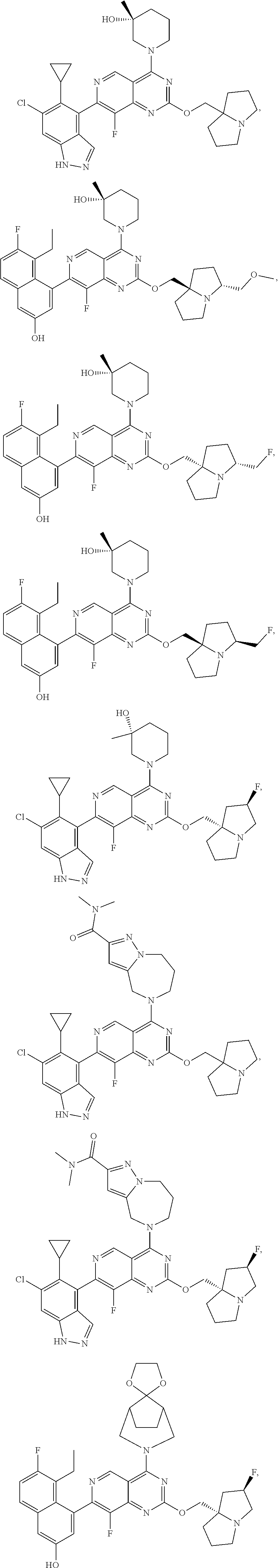

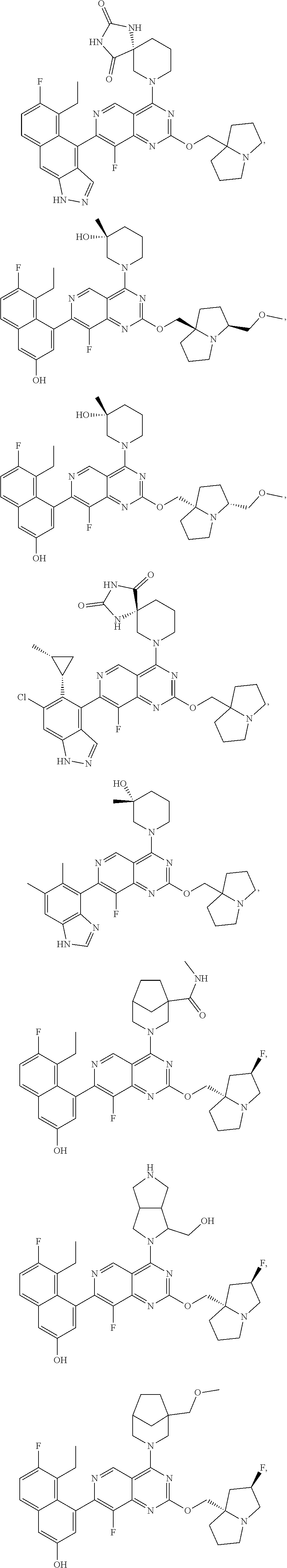

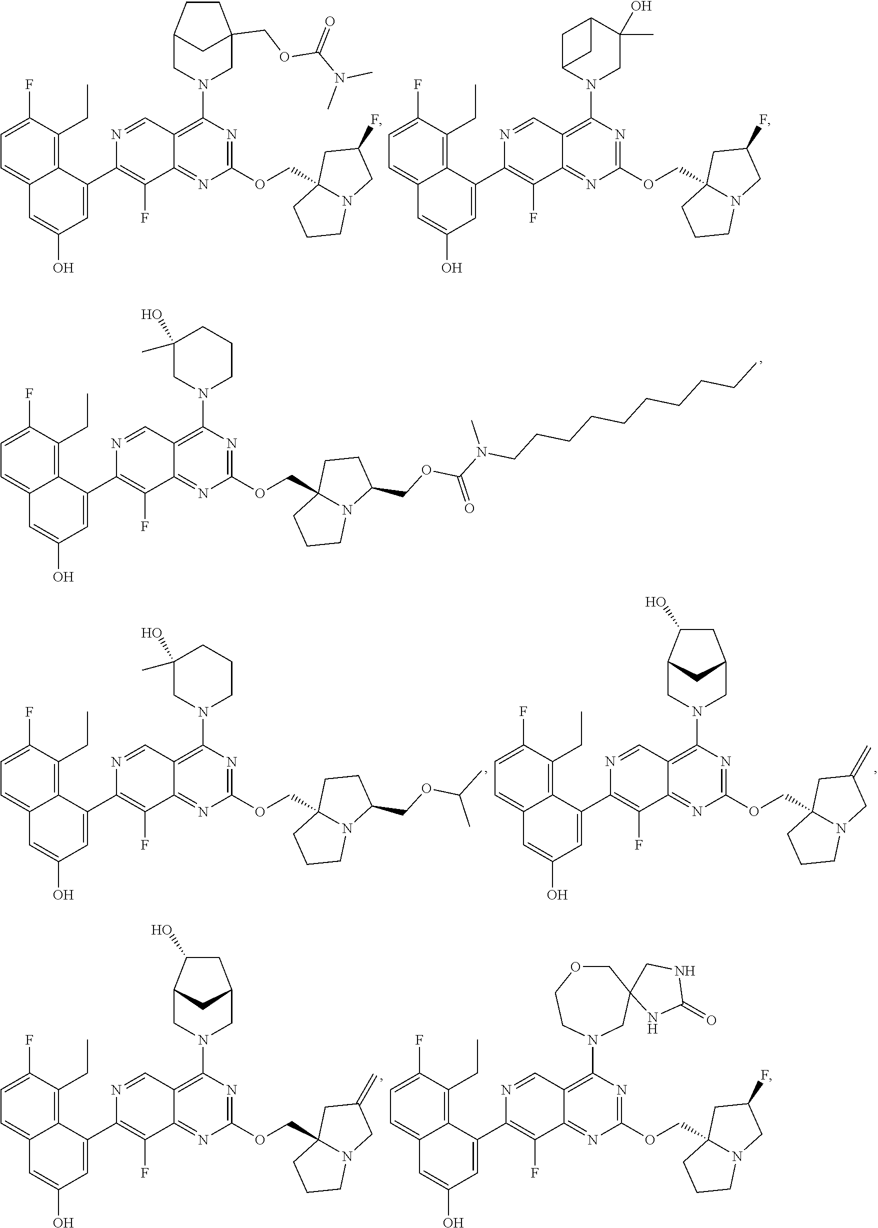

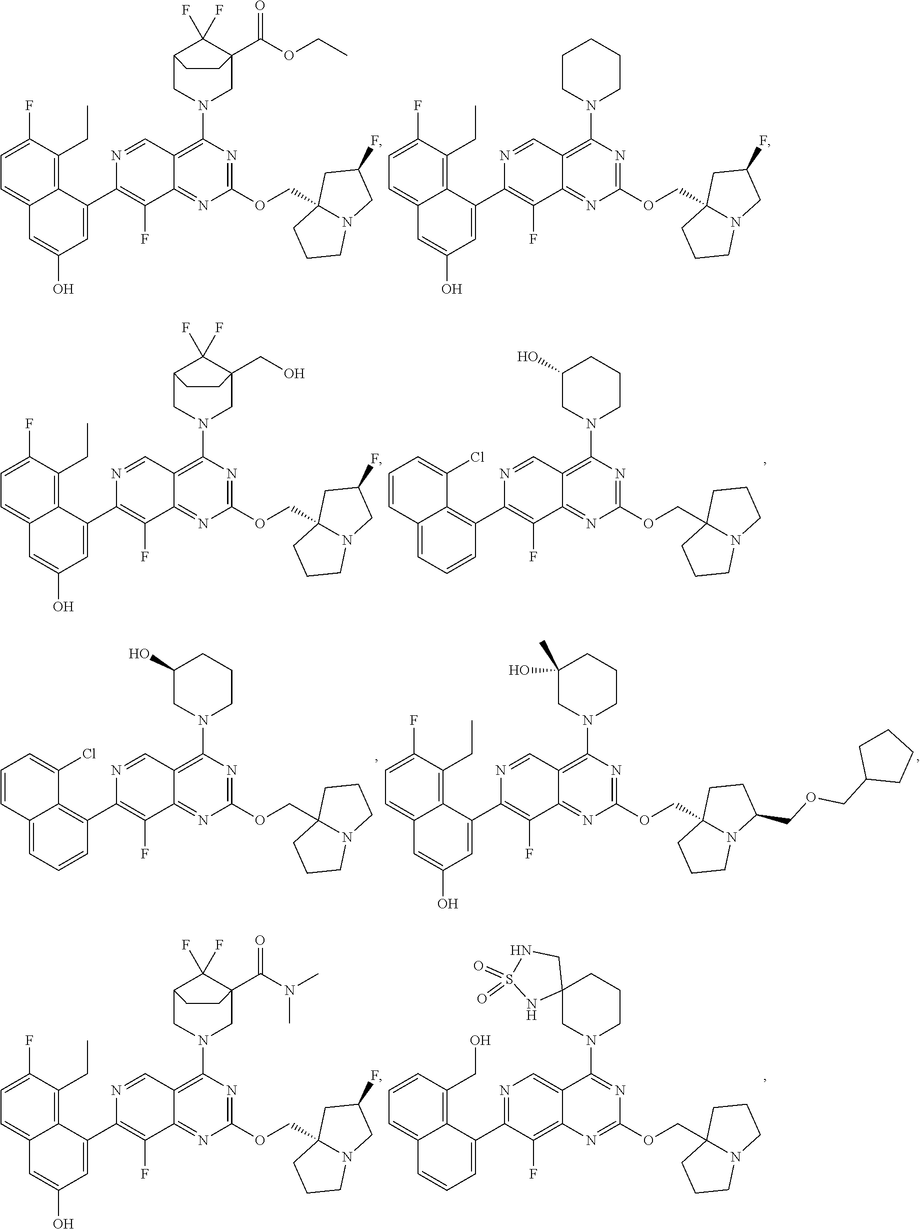

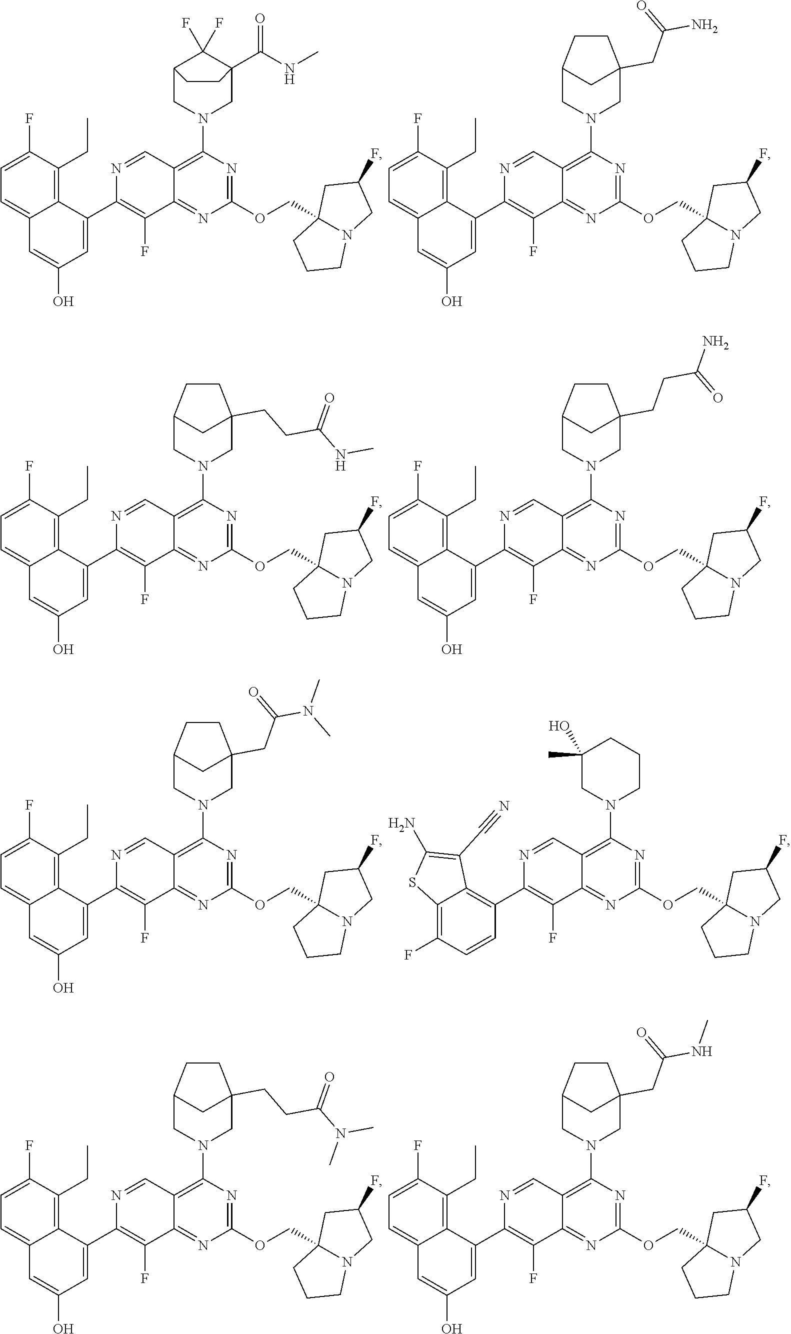

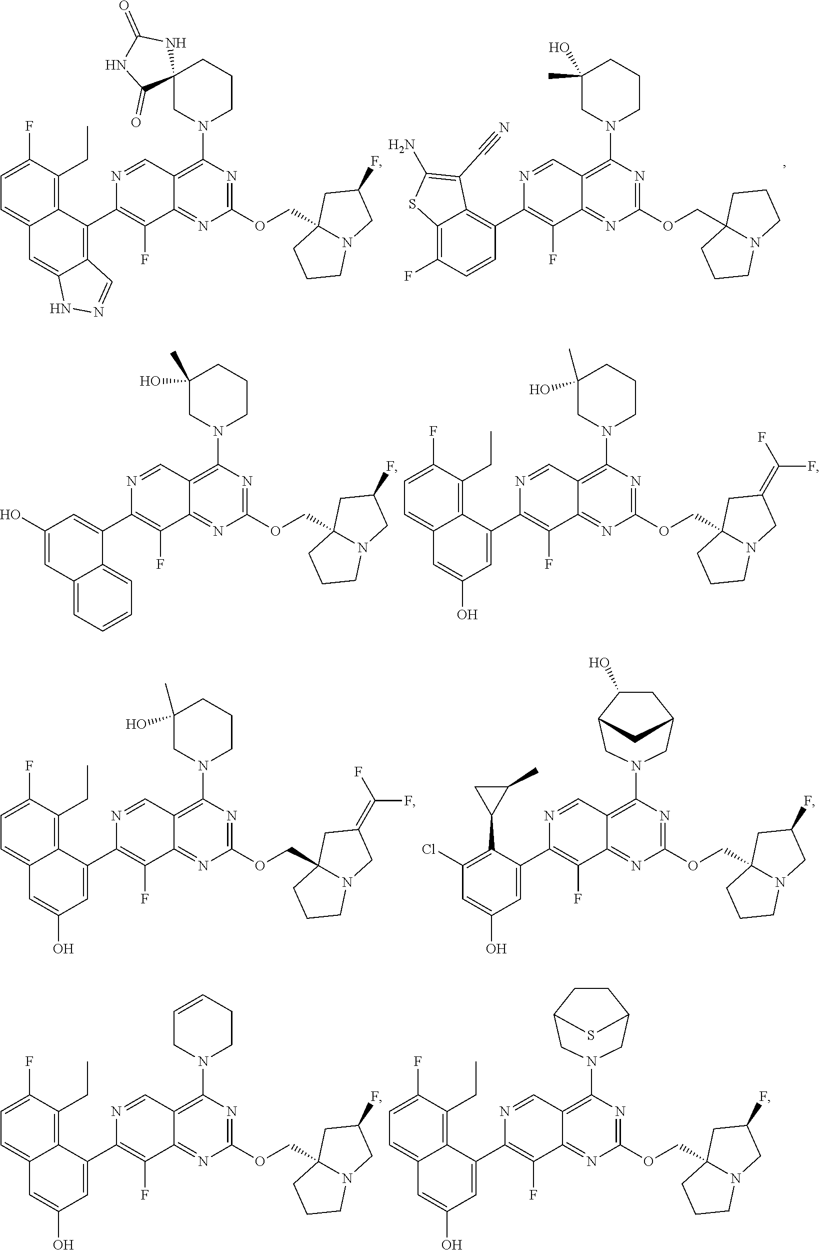

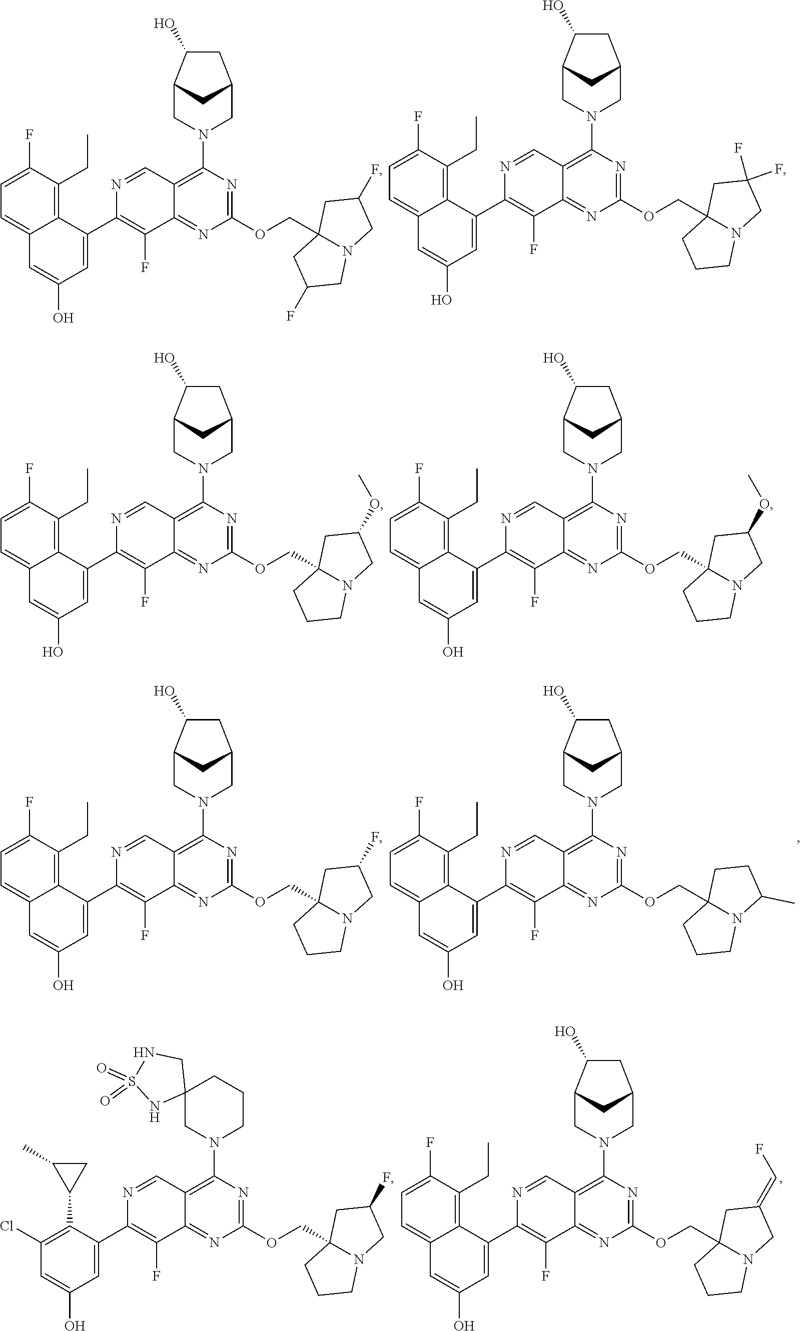

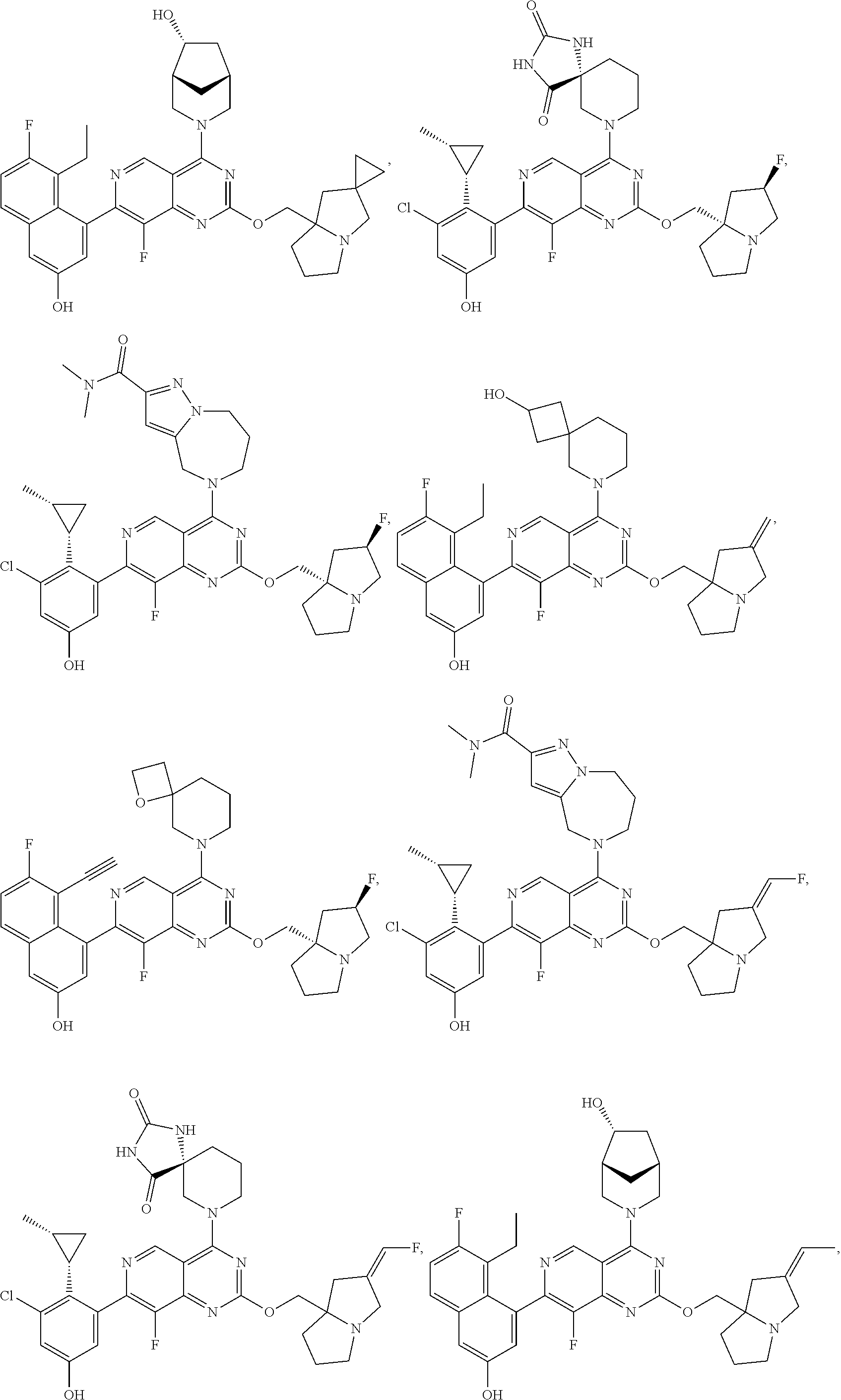

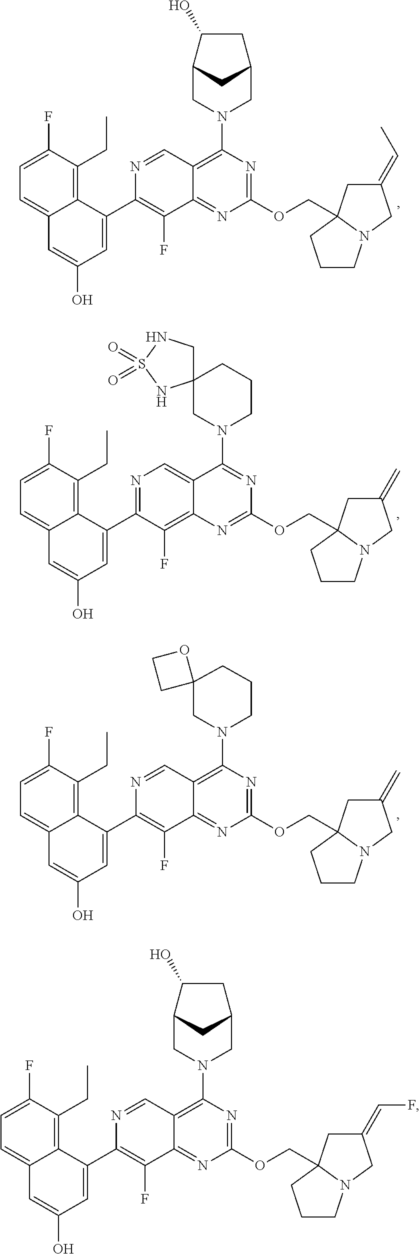 [SMILES: C/C=C1/CN2CCCC2(COc2nc(N3C[C@@H]4C[C@H](C3)[C@H](O)C4)c3cnc(-c4cc(O)cc5ccc(F)c(CC)c45)c(F)c3n2)C1 ; C=C1CN2CCCC2(COc2nc(N3CCCC4(CNS(=O)(=O)N4)C3)c3cnc(-c4cc(O)cc5ccc(F)c(CC)c45)c(F)c3n2)C1 ; C=C1CN2CCCC2(COc2nc(N3CCCC4(CCO4)C3)c3cnc(-c4cc(O)cc5ccc(F)c(CC)c45)c(F)c3n2)C1 ; CCc1c(F)ccc2cc(O)cc(-c3ncc4c(N5C[C@@H]6C[C@H](C5)[C@H](O)C6)nc(OCC56CCCN5C/C(=C\F)C6)nc4c3F)c12]

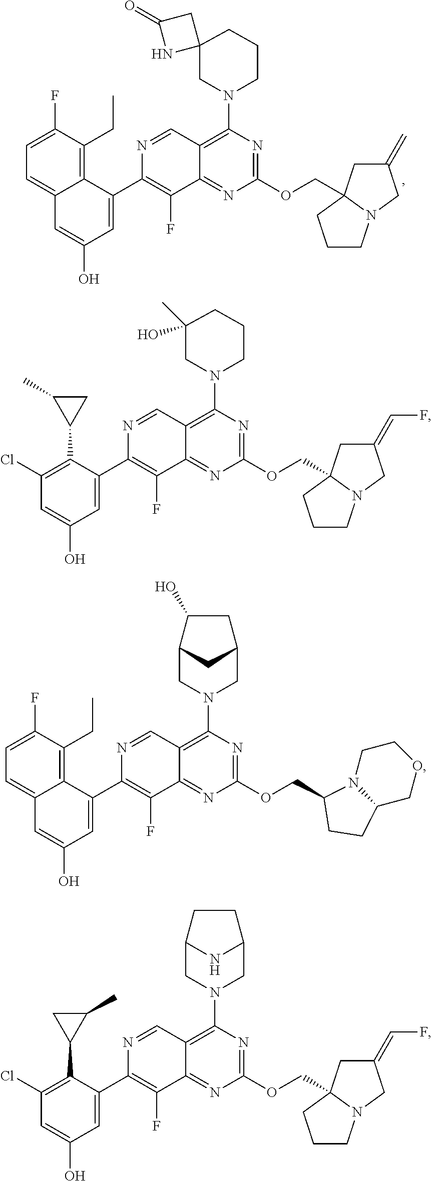 [SMILES: C=C1CN2CCCC2(COc2nc(N3CCCC4(CC(=O)N4)C3)c3cnc(-c4cc(O)cc5ccc(F)c(CC)c45)c(F)c3n2)C1 ; C[C@@H]1C[C@@H]1c1c(Cl)cc(O)cc1-c1ncc2c(N3CCC[C@@](C)(O)C3)nc(OC[C@@]34CCCN3C/C(=C\F)C4)nc2c1F ; CCc1c(F)ccc2cc(O)cc(-c3ncc4c(N5C[C@@H]6C[C@H](C5)[C@H](O)C6)nc(OC[C@@H]5CC[C@H]6COCCN65)nc4c3F)c12 ; C[C@@H]1C[C@@H]1c1c(Cl)cc(O)cc1-c1ncc2c(N3CC4CCC(C3)N4)nc(OC[C@@]34CCCN3C/C(=C\F)C4)nc2c1F]

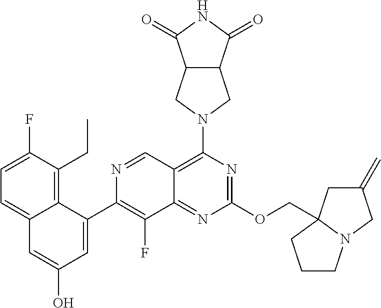 [SMILES: C=C1CN2CCCC2(COc2nc(N3CC4C(=O)NC(=O)C4C3)c3cnc(-c4cc(O)cc5ccc(F)c(CC)c45)c(F)c3n2)C1]

and pharmaceutically acceptable salts thereof.

Non-limiting examples of compounds of Formula (I) are selected from the group consisting of the compounds described in the below Examples, and pharmaceutically acceptable salts thereof.

In one embodiment, the compounds of Formula (I) include bis-hydrochloride, tris-hydrochloride, trifluoroacetic acid, bis-trifluoroacetic acid, and tris-trifluoracetic acid salts of the above compounds. The compounds of Formula (I) or pharmaceutically acceptable salt thereof may be formulated into pharmaceutical compositions.

### Pharmaceutical Compositions

In another aspect, the invention provides pharmaceutical compositions comprising a wild type Kras, Kras G12A, Kras G12C, Kras G12D, Kras G12R, Kras G12S, Kras G12V, Kras G13D and/or Kras Q61H inhibitor according to the invention and a pharmaceutically acceptable carrier, excipient, or diluent. Compounds of the invention may be formulated by any method well known in the art and may be prepared for administration by any route, including, without limitation, parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intranasal, intratracheal, intrarectal, subcutaneous, and topical administration. In certain embodiments, compounds of the invention are administered intravenously in a hospital setting. In one embodiment, administration may be by the oral route. In some embodiments, the provided pharmaceutical compositions may be administered to a subject in need of treatment by injection systemically, such as by intravenous injection; or by injection or application to the relevant site, such as by direct injection via syringe, or direct application to the site when the site is exposed in surgery; or by topical administration.

Parenteral administration can be by bolus injection or continuous infusion. Pharmaceutical compositions for injection may be presented in unit dosage form, e.g., in ampoules or in multi-dose containers, with an added preservative.

The provided pharmaceutical compositions can also be formulated as a depot preparation. Such long acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may, if desired, be presented in a vial, pack or a medical device, including but not limited to a dispenser device which may contain one or more unit dosage forms containing the active ingredient. In one embodiment the dispenser device can comprise a syringe having a single dose of the liquid formulation ready for injection. The syringe can be accompanied by instructions for administration.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 18^{th} Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z²—, wherein R is hydrogen, alkyl, or benzyl, and Z² is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting wild type KRas, KRas G12A, KRas G12C, KRas G12D, KRas G12R, KRas G12S, KRas G12V and/or KRas Q61H activity in a cell, comprising contacting the cell in which inhibition of wild type KRas, KRas G12A, KRas G12C, KRas G12D, KRas G12R, KRas G12S, KRas G12V and/or Q61H activity is desired with an effective amount of a compound of Formula (I), pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” wild type KRas, KRas G12A, KRas G12C, KRas G12D, KRas G12R, KRas G12S, KRas G12V, KRas G13D and/or KRas Q61H with a compound provided herein includes the administration of a compound provided herein to an individual or patient, such as a human, having wild type KRas or a KRas G12A, KRas G12C, KRas G12D, KRas G12R, KRas G12S, KRas G12V, KRas G13D and/or KRas Q61H mutation, as well as, for example, introducing a compound provided herein into a sample containing a cellular or purified preparation containing wild type KRas or a KRas G12A, KRas G12C, KRas G12D, KRas G12R, KRas G12S, KRas G12V, KRas G13D or KRas Q61H mutation.

In one embodiment, a cell in which inhibition of wild type KRas or KRas G12A, KRas G12C, KRas G12D, KRas G12R, KRas G12S, KRas G12V, KRas G13D and/or KRas Q61H activity is desired is contacted with an effective amount of a compound of Formula (I) or pharmaceutically acceptable salt thereof to negatively modulate the activity of one or more of wild type KRas or KRas G12A, KRas G12C, KRas G12D, KRas G12R, KRas G12S, KRas G12V, KRas G13D and KRas Q61H.

By negatively modulating the activity of one or more of wild type KRas or KRas G12A, KRas G12C, KRas G12D, KRas G12R, KRas G12S, KRas G12V, KRas G13D and KRas Q61H, the methods described herein are designed to inhibit undesired cellular proliferation resulting from enhanced wild type KRas or KRas G12A, KRas G12C, KRas G12D, KRas G12R, KRas G12S, KRas G12V, KRas G13D and/or KRas Q61H activity within the cell. The cells may be contacted in a single dose or multiple doses in accordance with a particular treatment regimen to affect the desired negative modulation of wild type KRas or KRas G12A, KRas G12C, KRas G12D, KRas G12R, KRas G12S, KRas G12V, KRas G13D and/or KRas Q61H. The ability of compounds to bind one or more of wild type KRas or KRas G12A, KRas G12C, KRas G12D, KRas G12R, KRas G12S, KRas G12V, KRas G13D and KRas Q61H may be monitored in vitro using well known methods, including those described in Examples A and B below. In addition, the inhibitory activity of exemplary compounds in cells may be monitored, for example, by measuring the inhibition of one or more of wild type KRas or KRas G12A, KRas G12C, KRas G12D, KRas G12R, KRas G12S, KRas G12V, KRas G13D and/or KRas Q61H activity of the amount of phosphorylated ERK, for example using the method described in Example C below.

In another aspect, methods of treating cancer in a patient in need thereof, comprising administering to said patient a therapeutically effective amount of a compound of Formula (I) or a pharmaceutically acceptable salt thereof, or a pharmaceutical composition comprising the compound or pharmaceutically acceptable salt thereof are provided.

The compositions and methods provided herein may be used for the treatment of a wild type KRas-associated or KRas G12A, KRas G12C, KRas G12D, KRas G12R, KRas G12S, KRas G12V, KRas G13D and/or KRas Q61H-associated cancer in a patient in need thereof, comprising administering to said patient a therapeutically effective amount of a compound of Formula (I), a pharmaceutically acceptable salt thereof, or a pharmaceutical composition comprising the compound or pharmaceutically acceptable salt thereof are provided. In one embodiment, the wild type KRas-associated or KRas G12A, KRas G12C, KRas G12D, KRas G12R, KRas G12S, KRas G12V, KRas G13D and/or KRas Q61H-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (I) or a pharmaceutically acceptable salt thereof, or a pharmaceutical composition thereof as defined herein for use in therapy.

Also provided herein is a compound of Formula (I) or a pharmaceutically acceptable salt thereof, or a pharmaceutical composition thereof as defined herein for use in the treatment of cancer.

Also provided herein is a compound of Formula (I) or a pharmaceutically acceptable salt thereof, or a pharmaceutical composition thereof for use in the inhibition of wild type KRas or KRas G12A, KRas G12C, KRas G12D, KRas G12R, KRas G12S, KRas G12V, KRas G13D and/or KRas Q61H.

Also provided herein is a compound of Formula (I) or a pharmaceutically acceptable salt thereof, or a pharmaceutical composition thereof as defined herein, for use in the treatment of wild type KRas-associated or a KRas G12A, KRas G12C, KRas G12D, KRas G12R, KRas G12S, KRas G12V, KRas G13D and/or KRas Q61H-associated disease or disorder.

Also provided herein is the use of a compound of Formula (I) or a pharmaceutically acceptable salt thereof, as defined herein in the manufacture of a medicament for the treatment of cancer.

Also provided herein is a use of a compound of Formula (I) or a pharmaceutically acceptable salt thereof, as defined herein in the manufacture of a medicament for the inhibition of activity of wild type KRas or KRas G12A, KRas G12C, KRas G12D, KRas G12R, KRas G12S, KRas G12V, KRas G13D and/or KRas Q61H.

Also provided herein is the use of a compound of Formula (I) or a pharmaceutically acceptable salt thereof, as defined herein, in the manufacture of a medicament for the treatment of wild type KRas-associated or a KRas G12A, KRas G12C, KRas G12D, KRas G12R, KRas G12S, KRas G12V, KRas G13D and/or KRas Q61H-associated disease or disorder.

Also provided herein is a method for treating cancer in a patient in need thereof, the method comprising (a) determining that cancer is associated with wild type KRas or a KRas G12A, KRas G12C, KRas G12D, KRas G12R, KRas G12S, KRas G12V, KRas G13D and/or KRas Q61H mutation (e.g., as determined using a regulatory agency-approved, e.g., FDA-approved, assay or kit); and (b) administering to the patient a therapeutically effective amount of a compound of Formula (I), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### REACTION SCHEMES AND EXAMPLES

The compounds of the present invention may be prepared from commercially available reagents using the synthetic methods and reaction schemes described herein, or using other reagents and conventional methods well known to those skilled in the art. For instance, compounds of the present invention may be prepared according to the reaction schemes and examples outlines below.

The compounds of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

The compounds of the present invention may be in anhydrous, solvated or hydrated forms, and all such forms are included within the scope of the invention.

The following Intermediates are intended to illustrate further certain embodiments of the invention and are not intended to limit the scope of the invention.

### Intermediate 1

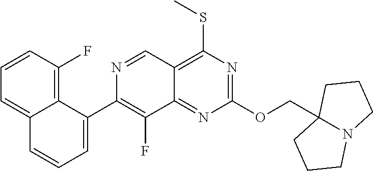 [SMILES: CSc1nc(OCC23CCCN2CCC3)nc2c(F)c(-c3cccc4cccc(F)c34)ncc12]

### 8-fluoro-7-(8-fluoronaphthalen-1-yl)-4-(methylthio)-2-((tetrahydro-1H-pyrrolizin-7a(5H)-yl)methoxy)pyrido[4,3-d]pyrimidine

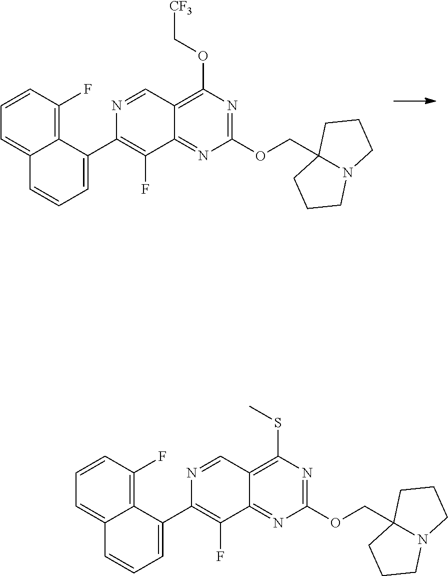 [SMILES: Fc1c(-c2cccc3cccc(F)c23)ncc2c(OCC(F)(F)F)nc(OCC34CCCN3CCC4)nc12 ; CSc1nc(OCC23CCCN2CCC3)nc2c(F)c(-c3cccc4cccc(F)c34)ncc12]

Step A. 8-fluoro-7-(8-fluoronaphthalen-1-yl)-4-(methylthio)-2-((tetrahydro-1H-pyrrolizin-7a(5H)-yl)methoxy)pyrido[4,3-d]pyrimidine: To a solution of 8-fluoro-7-(8-fluoronaphthalen-1-yl)-2-((hexahydro-1H-pyrrolizin-7a-yl)methoxy)-4-(2,2,2-trifluoroethoxy)pyrido[4,3-d]pyrimidine (500 mg, 1.0 equiv) in DCM (8 mL) was added NaSMe (119 mg, 1.8 equiv). The mixture was stirred at 20° C. for 2 hours. The mixture was filtered, concentrated and purified by column chromatography (SiO₂, petroleum ether/ethyl acetate 10:1 to 0:1) to afford the title compound (260 mg, 58% yield) as a white solid; ¹H NMR (400 MHZ, CHLOROFORM-d) δ=9.22 (s, 1H), 8.01 (br d, J=8.0 Hz, 1H), 7.75 (d, J=8.0 Hz, 1H), 7.67-7.61 (m, 1H), 7.60-7.56 (m, 1H), 7.46 (dt, J=5.2, 8.0 Hz, 1H), 7.16-7.08 (m, 1H), 4.40 (br s, 2H), 3.19 (br s, 2H), 2.79 (s, 3H), 2.75-2.64 (m, 2H), 2.18-2.07 (m, 2H), 1.98-1.86 (m, 4H), 1.77-1.66 (m, 2H). m/z=479.2.

### Intermediate 2

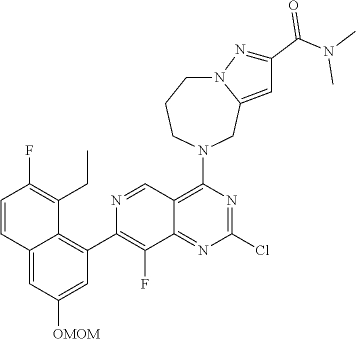

### 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide

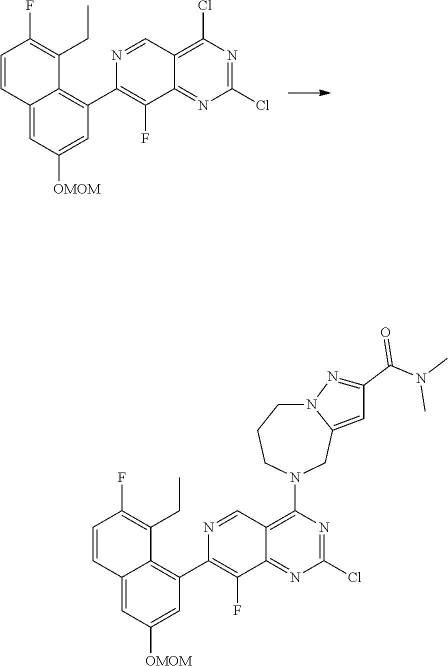

To a solution of 2,4-dichloro-7-(8-ethyl-7-fluoro-3-(methoxymethoxy)naphthalen-1-yl)-8-fluoropyrido[4,3-d]pyrimidine (4.55 g, 1.0 equiv) in DCM (50 mL) was added DIPEA (3.92 g, 3.0 equiv) and N,N-dimethyl-5,6,7,8-tetrahydro-4H-pyrazolo[1,5-a][1,4]diazepine-2-carboxamide (2.0 g, 0.95 equiv). The mixture was stirred at −40° C. for 0.5 hr. The reaction mixture was quenched by H₂O (20 mL) and extracted with ethyl acetate (20 mL×3). The combined organic layers were dried over anhydrous Na₂SO₄, concentrated and purified by reversed-phase flash chromatography (C18, 0.1% FA condition) to afford the title compound (5.00 g, 78% yield) as a white solid. LCMS (ESI, M+1): m/z=622.2.

### Intermediate 3

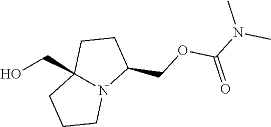 [SMILES: CN(C)C(=O)OC[C@@H]1CC[C@@]2(CO)CCCN12]

### ((3S,7aR)-7a-(hydroxymethyl)hexahydro-1H-pyrrolizin-3-yl)methyl dimethylcarbamate

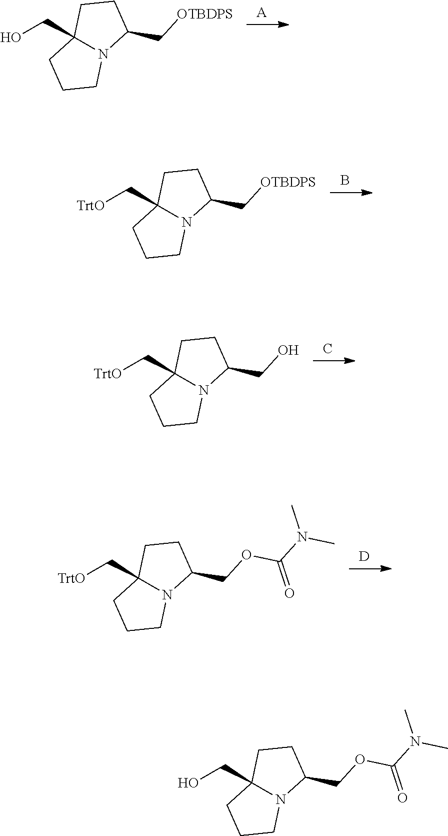

Step A. (3S,7aR)-3-(((tert-butyldiphenylsilyl)oxy)methyl)-7a-((trityloxy)methyl)hexahydro-1H-pyrrolizine: To a solution of ((3S,7aR)-3-(((tert-butyldiphenylsilyl)oxy)methyl)hexahydro-1H-pyrrolizin-7a-yl)methanol (2.00 g, 1.0 equiv) and TEA (1.24 g, 2.5 equiv) in DCM (20 mL) was added TrtCl (2.72 g, 2.0 equiv) at 0° C. The mixture was stirred at 15° C. for 10 hours. The mixture was diluted with water (200 mL) and extracted with ethyl acetate (2×100 mL). The organic layer was dried over Na₂SO₄ and concentrated to give a residue. The residue was purified by column chromatography [SiO₂, Petroleum ether/Ethyl acetate 20:1 to 5:1] to afford the title compound (3.40 g, crude) as a colorless oil; LCMS (ESI, M+1): m/z=652.5.

Step B. ((3S,7aR)-7a-((trityloxy)methyl)hexahydro-1H-pyrrolizin-3-yl)methanol: To a solution of (3S,7aR)-3-(((tert-butyldiphenylsilyl)oxy)methyl)-7a-((trityloxy)methyl)hexahydro-1H-pyrrolizine (3.40 g, 1.0 equiv) in DMF (20 mL) was added CsF (7.92 g, 10 equiv). The mixture was stirred at 25° C. for 6 hours. The mixture was diluted with water (200 mL) and extracted with ethyl acetate (2×50 mL). The organic layer was dried over Na₂SO₄ and concentrated to give a residue. The residue was purified by reversed phase flash chromatography [C18, 0.1% formic acid condition] to afford the title compound (980 mg, 45% yield) as a yellow oil; ¹H NMR (400 MHZ, CHLOROFORM-d) δ=7.53-7.19 (m, 15H), 3.52-3.24 (m, 2H), 2.96-2.60 (m, 5H), 2.03 (m, 1H), 1.93-1.83 (m, 1H), 1.82-1.49 (m, 6H); LCMS (ESI, M+1): m/z=414.2.

Step C. ((3S,7aR)-7a-((trityloxy)methyl)hexahydro-1H-pyrrolizin-3-yl)methyl dimethylcarbamate: To a solution of ((3S,7aR)-7a-((trityloxy)methyl)hexahydro-1H-pyrrolizin-3-yl)methanol (930 mg, 1.0 equiv) in THF (20 mL) was added NaH (360 mg, 60% purity, 4.0 equiv) at 0° C. The mixture was stirred at 0° C. for 10 minutes. To the mixture was added dimethylcarbamic chloride (484 mg, 2.0 equiv) at 0° C. The mixture was stirred at 15° C. for 20 hours. The mixture was quenched by water (20 mL) and extracted with ethyl acetate (50 mL). The organic layer was dried over Na₂SO₄ and concentrated to give a residue. The residue was purified by reversed phase flash chromatography [C18, 0.1% formic acid condition] to afford the title compound (1.00 g, 83% yield) as a yellow oil; LCMS (ESI, M+1): m/z=485.3.

Step D. ((3S,7aR)-7a-(hydroxymethyl)hexahydro-1H-pyrrolizin-3-yl)methyl dimethylcarbamate: To a solution of ((3S,7aR)-7a-((trityloxy)methyl)hexahydro-1H-pyrrolizin-3-yl)methyl dimethylcarbamate (620 mg, 1.0 equiv) in DCM (5 mL) was added TFA (1.46 g, 10 equiv) at 0° C. The mixture was stirred at 15° C. for 3 hours. The mixture was concentrated. To the residue was added MeOH (5 mL) and NaHCO₃ (300 mg). The mixture was filtered and concentrated to give a residue which was purified by column chromatography [Al₂O₃, Petroleum ether/Ethyl acetate 2:1 to 0:1, ethyl acetate/MeOH 12:1] to afford the title compound (220 mg, 71% yield) as a yellow oil; ¹H NMR (400 MHZ, METHANOL-d₄) δ=4.00 (dq, J=6.0, 10.8 Hz, 2H), 3.31-3.22 (m, 2H), 3.03-2.96 (m, 2H), 2.95-2.86 (m, 6H), 2.80 (td, J=5.2, 10.8 Hz, 1H), 2.09-1.46 (m, 8H).

### Intermediate 4

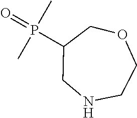

### dimethyl(1,4-oxazepan-6-yl)phosphine oxide

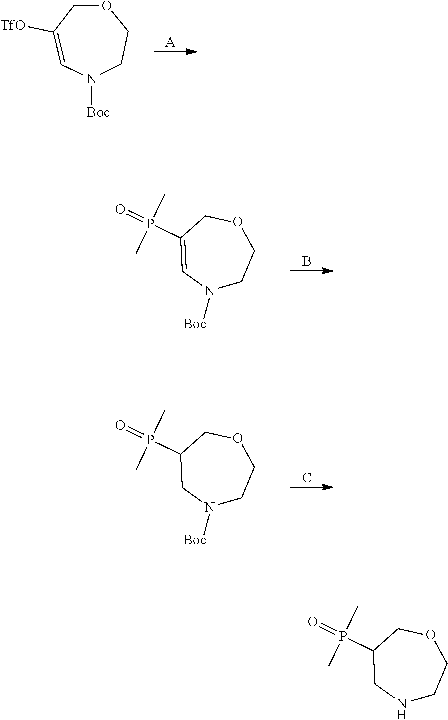

Step A: tert-butyl 6-(dimethylphosphoryl)-2,3-dihydro-1,4-oxazepine-4(7H)-carboxylate: A mixture of tert-butyl 6-(((trifluoromethyl) sulfonyl)oxy)-2,3-dihydro-1,4-oxazepine-4(7H)-carboxylate (350 mg, 1 equiv), methylphosphonoylmethane (86.5 mg, 1.1 equiv), Pd(PPh₃)₄ (58.2 mg, 0.05 equiv), Et₃N (204 mg, 2 equiv) and methylphosphonoylmethane (86.5 mg, 1.1 equiv) in MeCN (10 mL) was degassed and stirred at 90° C. for 10 hours under N₂ atmosphere. The reaction mixture was concentrated and purified by prep-HPLC (column: Waters Xbridge 150×25 mm×5 um; mobile phase: [water (ammonia hydroxide v/v)-ACN]; B %: 12%-42%, 9 min). ¹H NMR (400 MHZ, CHLOROFORM-d) δ=7.51 (d, J=15.6 Hz, 1H), 4.35 (br d, J=7.6 Hz, 2H), 3.99-3.84 (m, 4H), 1.59 (s, 3H), 1.55 (s, 3H), 1.52 (s, 9H)

Step B tert-butyl 6-(dimethylphosphoryl)-1,4-oxazepane-4-carboxylate: A mixture of tert-butyl 6-(dimethylphosphoryl)-2,3-dihydro-1,4-oxazepine-4(7H)-carboxylate (100 mg, 1.0 equiv), Pd/C (50 mg, 60% purity, 1.0 equiv) in MeOH (1.0 mL) was degassed and purged with H₂ for 3 times, and then the mixture was stirred at 25° C. for 2 hours under H₂ atmosphere. The reaction mixture was filtered with MeOH (20 mL) and concentrated under reduced pressure to afford the title compound (100 mg) as a white solid.

Step C dimethyl(1,4-oxazepan-6-yl)phosphine oxide: To a solution of tert-butyl 6-(dimethylphosphoryl)-1,4-oxazepane-4-carboxylate (100 mg, 1.0 equiv) in DCM (2.0 mL) was added TFA (411 mg, 10.0 equiv). The mixture was stirred at 25° C. for 1 hour. The reaction mixture was concentrated under reduced pressure to afford the title compound (50 mg) as a yellow oil.

### Intermediate 5

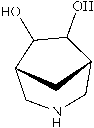

Three isomeric 3-azabicyclo[3.2.1]octane-6,7-diol (stereochemistry was arbitrarily assigned)

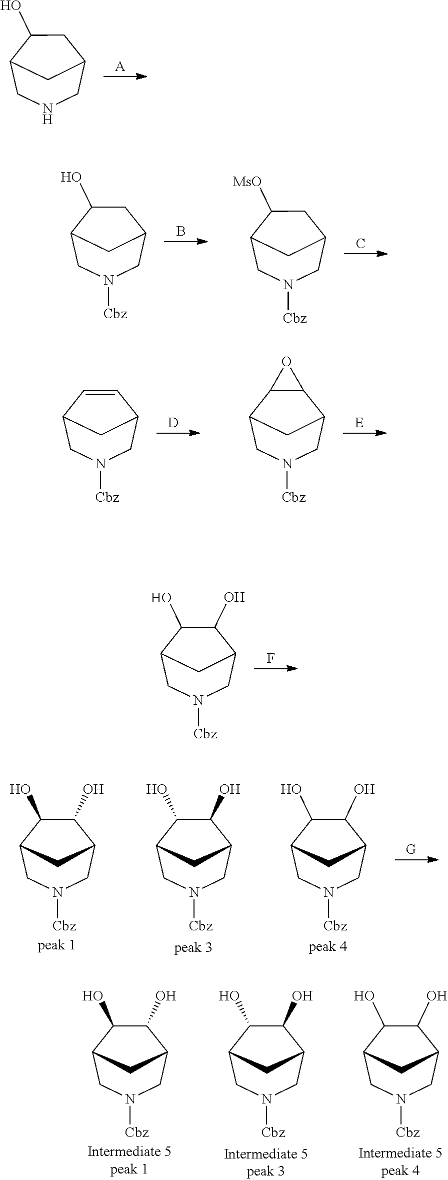

Step A. benzyl 6-hydroxy-3-azabicyclo[3.2.1]octane-3-carboxylate To a mixture of 3-azabicyclo[3.2.1]octan-6-ol (4.50 g, 1.0 equiv) and NaHCO₃ (11.9 g, 4.0 equiv) in EtOAc (54.0 mL) and water (36.0 mL) were added benzyl carbonochloridate (8.45 g, 1.4 equiv) and TBAB (1.14 g, 0.1 equiv) at 0° C. The reaction was stirred at 20° C. for 12 hours. The mixture was extracted with EtOAc (2×50 mL). The combined organic layers were dried over anhydrous sodium sulfate, concentrated, and purified by silica gel chromatography (petroleum ether/ethyl acetate 3:1 to ethyl acetate/methanol 30:1) to afford the title compound (8.30 g, 90% yield) as a colorless oil.

Step B. benzyl 6-((methylsulfonyl)oxy)-3-azabicyclo[3.2.1]octane-3-carboxylate: To a mixture of benzyl 6-hydroxy-3-azabicyclo[3.2.1]octane-3-carboxylate (7.50 g, 1.0 equiv) in pyridine (70 mL) was added methanesulfonyl chloride (5.84 g, 1.8 equiv) drop-wise at 0° C. The reaction was stirred at 20° C. for 0.5 hour. The mixture was diluted with water (300 mL) and extracted with EtOAc (3×100 mL). The combined organic layers were dried over anhydrous sodium sulfate, concentrated to afford the title compound (10 g, 94% yield) as a light-yellow solid.

Step C. benzyl 3-azabicyclo[3.2.1]oct-6-ene-3-carboxylate: To a mixture of benzyl 6-((methylsulfonyl)oxy)-3-azabicyclo[3.2.1]octane-3-carboxylate (9.00 g, 1.0 equiv) in 2,3,5-trimethylpyridine (25 mL) was added DBU (12.1 g, 3.0 equiv). The reaction was stirred at 170° C. for 3 hours. The mixture was acidified with 1N HCl (15 mL) and extracted with EtOAc (3×10 mL). The combined organic layers were washed with NaHCO₃ (10 mL), dried over anhydrous sodium sulfate, concentrated to afford the title compound (3.37 g, 51% yield) as a colorless oil; ¹H NMR (400 MHZ, chloroform-d) δ=7.40-7.28 (m, 5H), 6.02-5.91 (m, 2H), 5.12 (d, J=2.8 Hz, 2H), 3.85-3.70 (m, 2H), 3.09-2.95 (m, 2H), 2.70-2.63 (m, 1H), 2.61-2.54 (m, 1H), 2.10-2.01 (m, 1H), 1.60-1.57 (m, 1H).

Step D. benzyl 3-oxa-7-azatricyclo[3.3.1.02,4]nonane-7-carboxylate: To a mixture of benzyl 3-azabicyclo[3.2.1]oct-6-ene-3-carboxylate (3.47 g, 1.0 equiv) in DCM (70 mL) was added m-CPBA (6.15 g, 2.0 equiv). The reaction was stirred at 20° C. for 5 hours. The mixture was quenched by addition of saturated aqueous Na₂SO₃ (70 mL), neutralized with solid NaHCO₃ and extracted with DCM (2×70 mL). The organic layers were dried over anhydrous sodium sulfate and concentrated. The mixture was purified by silica gel chromatography (petroleum ether/ethyl acetate 20/1 to 3/1) to afford the title compound (2.50 g, 61% yield) as a light yellow solid; ¹H NMR (400 MHZ, chloroform-d) δ=7.43-7.30 (m, 5H), 5.19-5.08 (m, 2H), 4.02-3.88 (m, 2H), 3.40-3.28 (m, 2H), 3.16-3.04 (m, 2H), 2.45-2.31 (m, 2H), 1.64-1.54 (m, 1H), 1.20-1.13 (m, 1H).

Step E. benzyl 6,7-dihydroxy-3-azabicyclo[3.2.1]octane-3-carboxylate: To a mixture of benzyl 3-oxa-7-azatricyclo[3.3.1.02,4]nonane-7-carboxylate (2.50 g, 1.0 equiv) in THF (35 mL) was added H₂SO₄ (2 M in water, 72.3 mL, 5.0 equiv). The reaction was stirred at 60° C. for 12 hours. The mixture was basified with 40% NaOH under ice bath and extracted with EtOAc (4×50 mL). The combined organic layers were dried over anhydrous sodium sulfate, concentrated and purified by prep-HPLC [Phenomenex luna C18 250×50 mm×10 μm; A: water (TFA); B: ACN; B %: 14%-44% over 20 min]. The desired fractions were neutralized with solid K₂CO₃ and extracted with EtOAc (4×50 mL). The organic layers were dried over anhydrous sodium sulfate and concentrated to afford the title compound (1.1 g, 37% yield) as a yellow oil; SFC: Chiralpak AD-3 50×4.6 mm I.D., 3 μm; gradient elution: IPA (0.05% DEA) in CO₂ from 5% to 40%, t_{R}: 1.411 min, 1.468 min, 1.599 min, 1.707 min; LCMS (ESI, M+23): m/z=278.0.

Step F. Isomers of benzyl 6,7-dihydroxy-3-azabicyclo[3.2.1]octane-3-carboxylate: benzyl 6,7-dihydroxy-3-azabicyclo[3.2.1]octane-3-carboxylate (1.1 g) was separated by SFC [Daicel Chiralpak AD 250 mm×30 mm, 10 um; mobile phase: (0.1% NH₃·H₂O IPA); B %: 30%-30%, C8. 4; 244 min]. The four peaks were obtained. Sufficient purity for peak 2 was not achieved. SFC: Chiralpak AD-3 50×4.6 mm I.D., 3 μm; Gradient elution: 5% to 40% IPA (0.05% DEA) in CO₂, 3 mL/min, 220 nm, t_{R}: 1.713 min. (stereochemistry was arbitrarily assigned)

(1R,5S,6R,7R)-benzyl 6,7-dihydroxy-3-azabicyclo[3.2.1]octane-3-carboxylate (peak 1, 260 mg, 23% yield) as a colorless oil.

(1R,5S,6S,7S)-benzyl 6,7-dihydroxy-3-azabicyclo[3.2.1]octane-3-carboxylate (peak 3, 260 mg, 23% yield) as a colorless oil.

(1R,5S)-benzyl 6,7-dihydroxy-3-azabicyclo[3.2.1]octane-3-carboxylate (peak 4, 230 mg, 20% yield) as a colorless oil.

Step G. Isomers of 3-azabicyclo[3.2.1]octane-6,7-diol, Intermediate 5 peak 1: To a mixture of (1R,5S,6S,7S)-benzyl 6,7-dihydroxy-3-azabicyclo[3.2.1]octane-3-carboxylate (80.0 mg, 1.0 equiv) in MeOH (1.5 mL) was added Pd/C (30 mg, 10% purity, 1.0 equiv) under nitrogen atmosphere. The reaction was degassed and purged with hydrogen several times. The reaction was stirred under hydrogen (15 psi) at 20° C. for 2 hours. The mixture was filtered and concentrated to afford Intermediate 5 peak 1 (58.0 mg, 94% yield) as a white solid; LCMS (ESI, M+1): m/z=144.2.

Intermediate 5 peak 3 and Intermediate 5 peak 4 were obtained using the same procedure as described in Step G

### Intermediate 6

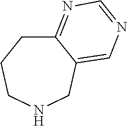 [SMILES: c1ncc2c(n1)CCCNC2]

### 6,7,8,9-tetrahydro-5H-pyrimido[5,4-c]azepine

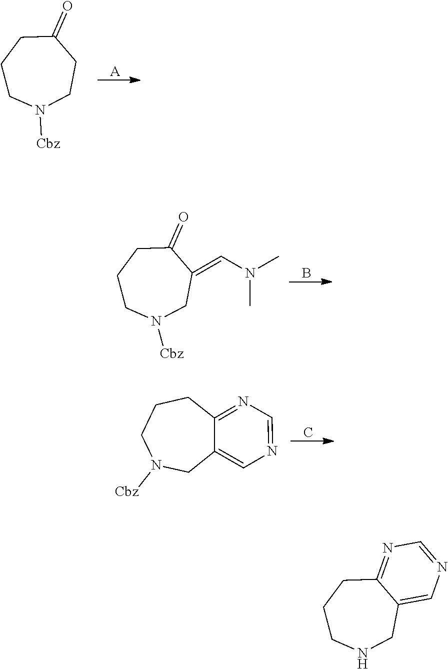

Step A. (E)-benzyl 3-((dimethylamino)methylene)-4-oxoazepane-1-carboxylate: A mixture of benzyl 4-oxoazepane-1-carboxylate (10 g, 1.0 equiv) in DMF-DMA (50 mL) was stirred at 100° C. for 12 hours. The mixture was concentrated to afford the title compound (12.0 g, crude) as a brown oil and used directly in the next step without further purification.

Step B. benzyl 8,9-dihydro-5H-pyrimido[5,4-c]azepine-6(7H)-carboxylate: To a solution of formimidamide (2.46 g, 1.1 equiv, AcOH) in EtOH (70 mL) was added EtONa (4.39 g, 3.0 equiv). The reaction was stirred at 20° C. for 0.5 hour. The (E)-benzyl 3-((dimethylamino)methylene)-4-oxoazepane-1-carboxylate (6.50 g, 1.0 equiv) was added into the mixture. The reaction was stirred at 80° C. for 2 hours. The mixture was diluted with H₂O (20 mL) and extracted with EtOAc (3×20 mL). The combined organic layers were washed with brine, dried over anhydrous sodium sulfate, concentrated, and purified with prep-HPLC (column: Phenomenex Luna C18 200×40 mm×10 um; mobile phase: [water (FA)-ACN]; B %: 25%-55%, 10 min) to afford the title compound (5.60 mg, 91% yield) as a yellow oil; LCMS (ESI, M+1): m/z=284.0.

Step C. 6,7,8,9-tetrahydro-5H-pyrimido[5,4-c]azepine: To a solution of benzyl 8,9-dihydro-5H-pyrimido[5,4-c]azepine-6(7H)-carboxylate (300 mg, 1.0 equiv) in MeOH (0.5 mL) was added Pd/C (300 mg, 10% purity) under nitrogen atmosphere. The suspension was degassed and purged with H₂ for 3 times. The reaction was stirred under H₂ (15 Psi) at 20° C. for 0.5 hour. The mixture was filtered and concentrated to afford the title compound (150 mg, 94% yield) as a yellow oil and used directly in the next step without further purification.

### Intermediate 7

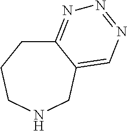 [SMILES: c1nnnc2c1CNCCC2]

### 6,7,8,9-tetrahydro-5H-[1,2,3]triazino[5,4-c]azepine

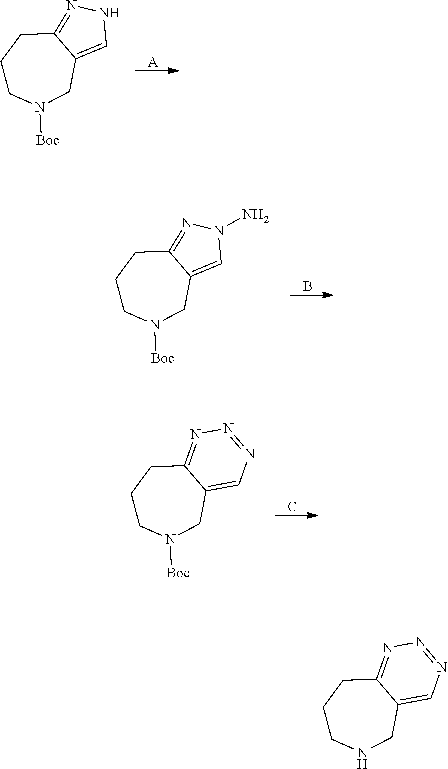

Step A. tert-butyl 2-amino-4,6,7,8-tetrahydropyrazolo[4,3-c]azepine-5 (2H)-carboxylate: To a solution of tert-butyl 4,6,7,8-tetrahydropyrazolo[4,3-c]azepine-5 (2H)-carboxylate (2.00 g, 1.0 equiv) in DMF (60 mL) was added t-BuONa (4.05 g, 5.0 equiv) at 0° C. in portions. The mixture was stirred at 0° C. for 0.5 hour. Amino hydrogen sulfate (2.86 g, 3.0 equiv) was added to the mixture at 0° C. in portions. The reaction was stirred at 0° C. for 2 hours. The mixture was quenched with water (200 mL) carefully at 0° C. and extracted with ethyl acetate (3×80 mL). The combined organic layers were washed with brine, dried over anhydrous sodium sulfate, concentrated and purified with prep-HPLC (column: Phenomenex luna C18 250×50 mm×10 μm; mobile phase: [water (FA)-ACN]; B %: 14%-44% over 20 min) to afford the title compound (250 mg, 12% yield) as a yellow solid; LCMS (ESI, M−55, M+1): m/z=196.8, 252.9.

Step B. tert-butyl 8,9-dihydro-5H-[1,2,3]triazino[5,4-c]azepine-6(7H)-carboxylate: To a solution of tert-butyl 2-amino-4,6,7,8-tetrahydropyrazolo[4,3-c]azepine-5 (2H)-carboxylate (200 mg, 1.0 equiv) in DCM (3 mL) and H₂O (3 mL) was added NaIO₄ (254 mg, 1.5 equiv). The reaction was stirred at 20° C. for 2 hours. The mixture was quenched with saturated aqueous Na₂S₂O₃ (10 mL) at 0° C. and extracted with DCM (2×5 mL). The combined organic layers were washed with brine, dried over anhydrous sodium sulfate, concentrated and purified by reversed phase flash chromatography (C18, 0.1% formic acid condition) to afford the title compound (120 mg, 60% yield) as a yellow solid; ¹H NMR (400 MHZ, DMSO-d₆) δ=9.15-8.94 (m, 1H), 4.48 (br s, 2H), 3.67 (br s, 2H), 3.31-3.27 (m, 2H), 1.90-1.65 (m, 2H), 1.34-1.22 (m, 9H).

Step C. 6,7,8,9-tetrahydro-5H-[1,2,3]triazino[5,4-c]azepine: A solution of tert-butyl 8,9-dihydro-5H-[1,2,3]triazino[5,4-c]azepine-6(7H)-carboxylate (60.0 mg, 1.0 equiv) in HCOOH (1 mL) was stirred at 20° C. for 0.5 hour. The mixture was concentrated to afford the title compound (50 mg, crude) as a yellow oil and used for the next step directly; LCMS (ESI, M+1): m/z=151.2.

### Intermediate 8

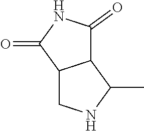

### 4-methyltetrahydropyrrolo[3,4-c]pyrrole-1,3(2H,3aH)-dione

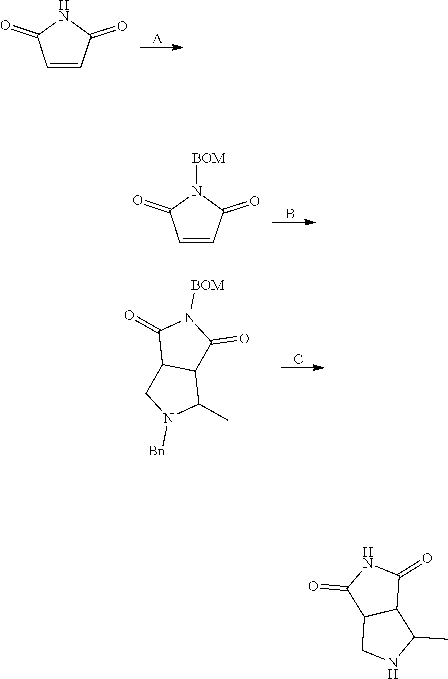

Step A. 1-((benzyloxy)methyl)-1H-pyrrole-2,5-dione: To a solution of pyrrole-2,5-dione (5.00 g, 1.0 equiv) in THF (100 mL) were added DIPEA (20.0 g, 3.0 equiv) and ((chloromethoxy)methyl)benzene (16.1 g, 2.0 equiv) at 0° C. The reaction was stirred at 0° C. for 2 hours. The reaction mixture was quenched by water (100 mL) and extracted with ethyl acetate (3×100 mL). The combined organic layers were dried over Na₂SO₄, concentrated and purified by column chromatography (SiO₂, petroleum ether/ethyl acetate 1:0 to 3:1) to afford the title compound (4.00 g, 36% yield) as a white solid; ¹H NMR (400 MHZ, chloroform-d) δ=7.33-7.28 (m, 3H), 7.27-7.19 (m, 2H), 6.66 (s, 2H), 4.98 (s, 2H), 4.54 (s, 2H).

Step B. 5-benzyl-2-((benzyloxy)methyl)-4-methyltetrahydropyrrolo[3,4-c]pyrrole-1,3(2H,3aH)-dione: To a solution of 1-((benzyloxy)methyl)-1H-pyrrole-2,5-dione (1.00 g, 1.0 equiv) in THF (100 mL) was added N-benzyl-1-(trimethylsilyl) methanamine (1.78 g, 2.0 equiv) and acetaldehyde (1.01 g, 40% purity, 2.0 equiv). The reaction was stirred at 70° C. for 7 hours. The reaction mixture was concentrated and purified by column chromatography (SiO₂, petroleum ether/ethyl acetate 1:0 to 0:1) to afford the title compound (225 mg, 13% yield) as a colorless oil; ¹H NMR (400 MHZ, chloroform-d) δ=7.26-7.09 (m, 10H), 4.93 (s, 2H), 4.53 (s, 2H), 3.69-3.64 (m, 1H), 3.33 (d, J=13.2 Hz, 1H), 3.13-3.03 (m, 2H), 3.01-2.94 (m, 1H), 2.81-2.71 (m, 1H), 2.66-2.60 (m, 1H), 1.14 (d, J=6.4 Hz, 3H).

Step C. 4-methyltetrahydropyrrolo[3,4-c]pyrrole-1,3(2H,3aH)-dione: To a mixture of 5-benzyl-2-((benzyloxy)methyl)-4-methyltetrahydropyrrolo[3,4-c]pyrrole-1,3(2H,3aH)-dione (400 mg, 1.0 equiv) in ethanol (8 mL) were added NH₃·MeOH (12 M, 4 mL, 44 equiv) and Pd(OH)₂/C (50.0 mg, 10% purity) under N₂ atmosphere. The suspension was degassed and purged with H₂ for 3 times. The reaction was stirred under H₂ (50 Psi) at 50° C. for 24 hours. The reaction mixture was filtered under N₂. The filtrate was concentrated and purified by prep-HPLC [Phenomenex luna C18 150×25 mm×10 μm; A: water (FA), B: ACN, B %: 1%-25% over 15 min] to afford the title compound (159 mg, 94% yield) as a white solid; ¹H NMR (400 MHZ, dimethylsulfoxide-d₆) δ=8.20 (br s, 1H), 3.43-3.11 (m, 3H), 3.06-2.61 (m, 3H), 1.15-0.99 (m, 3H).

### Intermediate 9

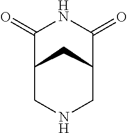 [SMILES: O=C1NC(=O)[C@H]2CNC[C@@H]1C2]

### (1R,5S)-3,7-diazabicyclo[3.3.1]nonane-2,4-dione

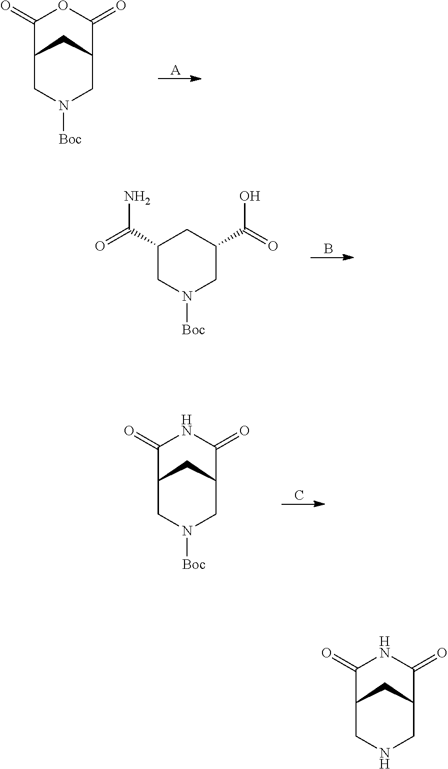

Step A. (3S,5R)-1-(tert-butoxycarbonyl)-5-carbamoylpiperidine-3-carboxylic acid: A solution of tert-butyl (1R,5S)-2,4-dioxo-3-oxa-7-azabicyclo[3.3.1]nonane-7-carboxylate (500 mg, 1.96 mmol, 1.00 eq) in NH₃·H₂O (32.5 g, 260 mmol, 35.7 mL, 28.0% purity, 132 eq), was stirred at 80° C. for 4 hrs under N₂ atmosphere. The solvent was removed under reduced pressure to give the crude (3S,5R)-1-(tert-butoxycarbonyl)-5-carbamoylpiperidine-3-carboxylic acid (450 mg, crude) as a white solid.

Step B. tert-butyl (1R,5S)-6,8-dioxo-3,7-diazabicyclo[3.3.1]nonane-3-carboxylate: To a solution of (3S,5R)-1-(tert-butoxycarbonyl)-5-carbamoylpiperidine-3-carboxylic acid (3.30 g, 12.1 mmol, 1.00 eq) in THF (10.0 mL) was added CDI (7.86 g, 48.5 mmol, 4.00 eq). The reaction was stirred at 75° C. for 12 hrs under N₂. The mixture was poured into ice water (20 mL), and then 1 N HCl was added until pH was adjusted to 4. The mixture was extracted with DCM (10.0 mL×3). The combined organic layers were dried over Na₂SO₄, filtered, and concentrated under reduced pressure to give a residue. The residue was triturated with MTBE (3V) at 25° C. for 30 min to give product compound 7 (mmol, 14.6% yield) as a white solid. ¹H NMR: (400 MHZ, DMSO) δ10.9 (s, 1H), 4.12-4.10 (m, 2H), 3.07-3.04 (m, 2H), 2.62 (s, 2H), 3.30-2.27 (m, 1H), 1.87-1.83 (m, 1H), 1.34 (s, 9H).

Step C. (1R,5S)-3,7-diazabicyclo[3.3.1]nonane-2,4-dione: To a solution of tert-butyl (1R,5S)-6,8-dioxo-3,7-diazabicyclo[3.3.1]nonane-3-carboxylate (250 mg, 983 μmol, 1.00 eq) in DCM (2.50 mL) was added TFA (673 mg, 5.90 mmol, 437 μL, 6.00 eq) at 0° C. The reaction was stirred at 20° C. for 5 hrs. The mixture was concentrated in vacuum. The crude product was triturated with EtOAc (10.0 ml) at 25° C. for 40 min to give (140 mg, 889 μmol, 90.4% yield, 98.0% purity) as a white solid. LCMS (ESI, M+1): m/z=155.1 (M+H)⁺

### Intermediate 10

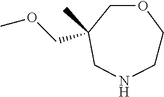

### (R)-6-(methoxymethyl)-6-methyl-1,4-oxazepane

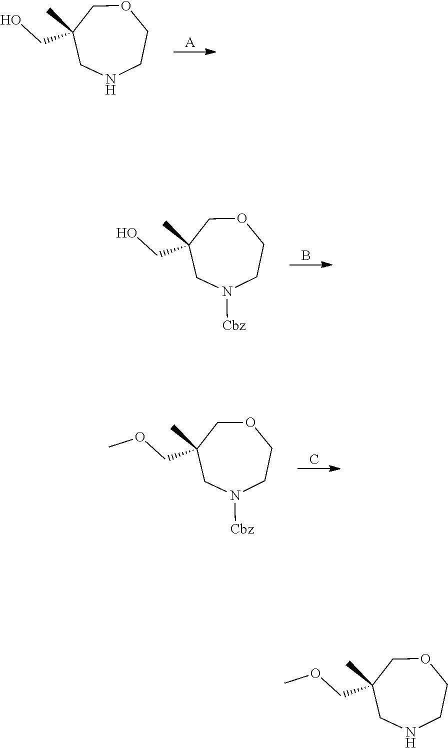

Step A. (R)-benzyl 6-(hydroxymethyl)-6-methyl-1,4-oxazepane-4-carboxylate: A mixture of [(6R)-6-methyl-1,4-oxazepan-6-yl]methanol (300 mg, 1.0 equiv), CbzCl (881 mg, 2.5 equiv), K₂CO₃ (857 mg, 3.0 equiv) in EtOAc (4 mL) and H₂O (4 mL) was stirred at 28° C. for 5 hours. The mixture was extracted with EtOAc 15 mL (5 mL×3), washed with brine, dried over Na₂SO₄, filtered and purified by column chromatography (SiO₂, Petroleum ether/Ethyl acetate 20:1 to 3:1) and lyophilized to afford the title compound (300 mg, 44% yield) as a yellow solid; ¹H NMR (400 MHZ, METHANOL-d₄) δ=7.44-7.26 (m, 5H), 5.16-5.11 (m, 2H), 3.80-3.68 (m, 2H), 3.65-3.57 (m, 2H), 3.56-3.50 (m, 1H), 3.46-3.42 (m, 1H), 3.42-3.36 (m, 2H), 3.36-3.32 (m, 1H), 3.32-3.28 (m, 2H), 0.89-0.83 (m, 3H) LCMS (ESI, M+1): m/z=280.2.

Step B. (R)-benzyl 6-(methoxymethyl)-6-methyl-1,4-oxazepane-4-carboxylate: To a solution of benzyl (6R)-6-(hydroxymethyl)-6-methyl-1,4-oxazepane-4-carboxylate (300 mg, 1.0 equiv) in THF (8 mL) was added NaH (85.9 mg, 60% purity, 2.0 equiv) at 0° C. The mixture was stirred for 0.5 hour, then CH₃I (305 mg, 2.0 equiv) was added dropwise and the mixture was stirred at 0° C. for 2 hours. The mixture was quenched by H₂O (3 mL) at 0° C., filtered and purified by column chromatography (SiO₂, Petroleum ether/Ethyl acetate 10:1 to 3:1) to afford the title compound (100 mg, 27% yield) as a white solid; ¹H NMR (400 MHZ, METHANOL-d₄) δ=7.44-7.26 (m, 5H), 5.20-5.08 (m, 2H), 3.77-3.69 (m, 3H), 3.63-3.51 (m, 5H), 3.38-3.32 (m, 2H), 3.21-3.16 (m, 2H), 3.16-3.09 (m, 1H), 0.87 (br d, J=18.0 Hz, 3H) LCMS (ESI, M+1): m/z=294.1

Step C. (R)-6-(methoxymethyl)-6-methyl-1,4-oxazepane: A mixture of benzyl (6R)-6-(methoxymethyl)-6-methyl-1,4-oxazepane-4-carboxylate (50 mg, 1.0 equiv), Pd/C (10 mg, 0.1 equiv) in MeOH (5 mL) was degassed and purged with H₂ for 3 times, and then the mixture was stirred at 20° C. for 2 hours under H₂ atmosphere (15 psi). The mixture was filtered and concentrated under reduced pressure to afford the title (20 mg, 74% yield) as a yellow oil; ¹H NMR (400 MHZ, CHLOROFORM-d) δ=3.70-3.40 (m, 5H), 3.34 (s, 3H), 3.18 (br s, 2H), 3.06-2.35 (m, 4H), 0.95-0.88 (m, 3H)

### Intermediate 11

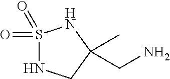

### 3-(aminomethyl)-3-methyl-1,2,5-thiadiazolidine 1,1-dioxide

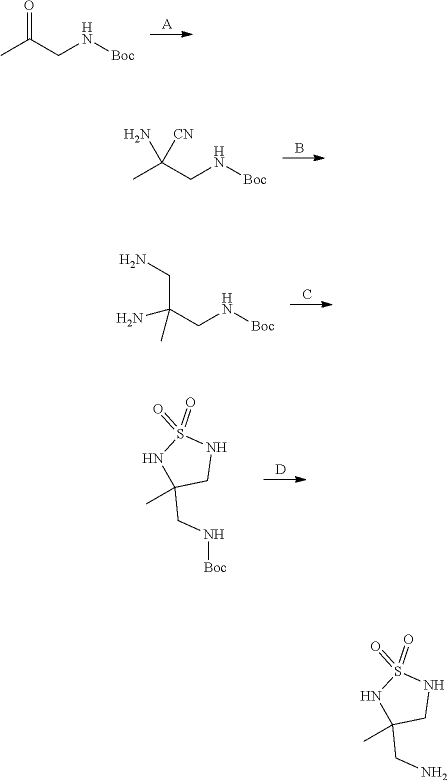

Step A. tert-butyl (2-amino-2-cyanopropyl)carbamate: To a solution of tert-butyl (2-oxopropyl)carbamate (1.00 g, 1.0 equiv) in MeOH (10 mL) were added Ti(i-PrO)₄ (1.64 g, 1.0 equiv) and NH₃·MeOH (7.00 M, 2.0 equiv). TMSCN (1.15 g, 2.0 equiv) was added dropwise to the resulting mixture at 0° C. The reaction was stirred at 25° C. for 16 hours. The mixture was diluted with water (50 mL), filtered and extracted with ethyl acetate (3×100 mL). The combined organic phase was dried over Na₂SO₄, filtered, concentrated and purified by flash silica gel chromatography (0-100% ethyl acetate/petroleum ether) to afford the title compound (800 mg, 69% yield) as a white solid; ¹H NMR (400 MHZ, dimethylsulfoxide-d6) δ=7.16 (br t, J=5.6 Hz, 1H), 3.10 (br d, J=6.0 Hz, 2H), 2.56 (s, 2H), 1.39 (s, 9H), 1.28 (s, 3H).

Step B. tert-butyl (2,3-diamino-2-methylpropyl)carbamate: To a mixture of tert-butyl (2-amino-2-cyanopropyl)carbamate (200 mg, 1.0 equiv) and in MeOH (2 mL) and NH₃·MeOH (0.5 mL) was added Raney-Ni (257 mg, 3.0 equiv). The reaction was degassed and purged with hydrogen for 3 times. The reaction was stirred at 25° C. for 5 hours under hydrogen (15 psi) atmosphere. The mixture was filtered and concentrated to afford the title compound (80.0 mg, crude) as a yellow oil.

Step C. tert-butyl ((3-methyl-1,1-dioxido-1,2,5-thiadiazolidin-3-yl)methyl)carbamate: To a solution of tert-butyl (2,3-diamino-2-methylpropyl)carbamate (80.0 mg, 1.0 equiv) in pyridine (3 mL) was added a solution of sulfamide (37.8 mg, 1.0 equiv) in pyridine (3 mL) dropwise at 25° C. The reaction was stirred at 115° C. for 20 hours. The mixture was filtered, concentrated, and purified by prep-TLC (dichloromethane/methanol 10:1) to afforded title compound (50 mg, 19% yield) as a yellow oil; 1H NMR (400 MHZ, dimethylsulfoxide-d6) δ=7.06 (br t, J=7.2 Hz, 1H), 6.92 (br t, J=5.6 Hz, 1H), 6.76 (s, 1H), 3.22 (br dd, J=7.6, 11.6 Hz, 1H), 3.15-3.08 (m, 1H), 3.06-2.97 (m, 1H), 2.96-2.88 (m, 1H), 1.38 (s, 9H), 1.16 (s, 3H).

Step D. 3-(aminomethyl)-3-methyl-1.2.5 thiadiazolidine 1,1-dioxide: To a solution of tert-butyl ((3-methyl-1,1-dioxido-1,2,5-thiadiazolidin-3-yl)methyl)carbamate (40 mg, 1.0 equiv) in dichloromethane (0.5 mL) was added dropwise HCl·dioxane (4 M, 1 mL 1.0 equiv) at 0° C. The reaction was stirred at 25° C. for 1 hour. The mixture was concentrated to afford the title compound (30.0 mg, crude, HCl) as a brown solid.

### Intermediate 12

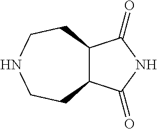 [SMILES: O=C1NC(=O)[C@@H]2CCNCC[C@H]12]

### (3aR,8aS)-hexahydropyrrolo[3,4-d]azepine-1,3(2H,3aH)-dione

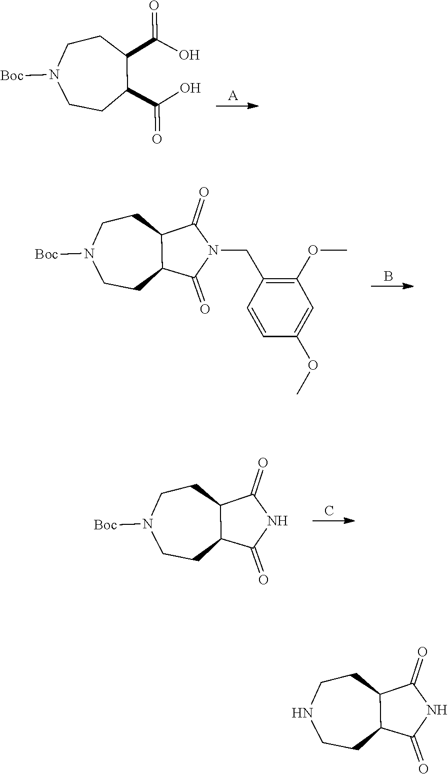

Step A. (3aR,8aS)-tert-butyl 2-(2,4-dimethoxybenzyl)-1,3-dioxooctahydropyrrolo[3,4-d]azepine-6 (2H)-carboxylate: To a solution of 1-tert-butoxycarbonylazepane-4,5-dicarboxylic acid (500, 1.0 equiv) in MeCN (5 mL) was added di(1H-imidazol-1-yl) methanone (564 mg, 2.0 equiv) at 20° C. After addition, the mixture was stirred at 50° C. for 1 hour, and (2,4-dimethoxyphenyl) methanamine (291 mg, 1.0 equiv) was added at 20° C. The resulting mixture was stirred at 90° C. for 18 hours. The mixture was concentrated under reduced pressure to give a residue. The residue was purified by column chromatography (SiO₂, petroleum ether/ethyl acetate 10:1 to 1:1) to afford the title compound (500 mg, 69% yield) as a colorless oil; LCMS (ESI, M−55): m/z=419.2

Step B. (3aR,8aS)-tert-butyl 1,3-dioxooctahydropyrrolo[3,4-d]azepine-6 (2H)-carboxylate: To a solution of tert-butyl (3aR,8aS)-2-[(2,4-dimethoxyphenyl)methyl]-1,3-dioxo-3a,4,5,7,8,8a-hexahydropyrrolo[3,4-d]azepine-6-carboxylate (100 mg, 1.0 equiv) in ACN (5 mL) was added CAN (262 mg, 2.0 equiv) in water (0.5 mL) at 0° C. Then the mixture was stirred at 20° C. for 0.5 hour. The mixture was diluted with ethyl acetate (20 mL) and washed with brine (10 mL×2). The organic layers were dried over anhydrous sodium sulfate, filtered and concentrated under reduced pressure to give a residue. The residue was purified by prep-TLC (SiO₂, petroleum ether/ethyl acetate 1:1) to afford the title compound (25 mg, 39% yield) as a colorless oil; ¹H NMR (400 MHZ, CHLOROFORM-d) δ=8.07 (br s, 1H), 3.82-3.66 (m, 1H), 3.61-3.48 (m, 1H), 3.47-3.36 (m, 1H), 3.30 (ddd, J=6.0, 8.0, 14.2 Hz, 1H), 2.76-2.59 (m, 2H), 2.56-2.35 (m, 2H), 1.76-1.62 (m, 2H), 1.50-1.42 (m, 9H).

Step C. (3aR,8aS)-hexahydropyrrolo[3,4-d]azepine-1,3(2H,3aH)-dione: To a solution of tert-butyl (3aR,8aS)-1,3-dioxo-3a,4,5,7,8,8a-hexahydropyrrolo[3,4-d]azepine-6-carboxylate (25 mg, 1.0 equiv) in DCM (1.5 mL) was added trifluoroacetic acid (770 mg, 72.5 equiv). The mixture was stirred at 20° C. for 0.5 hour. The mixture was concentrated under reduced pressure to afford the title compound (25 mg, 95% yield, TFA) as a white solid. The crude product was used for the next step without further purification; LCMS (ESI, M+1): m/z=169.2

### Intermediate 13A

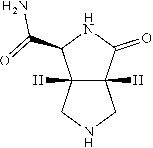 [SMILES: NC(=O)[C@H]1NC(=O)[C@@H]2CNC[C@H]12]

### (1S,3aS,6aR)-3-oxooctahydropyrrolo[3,4-c]pyrrole-1-carboxamide

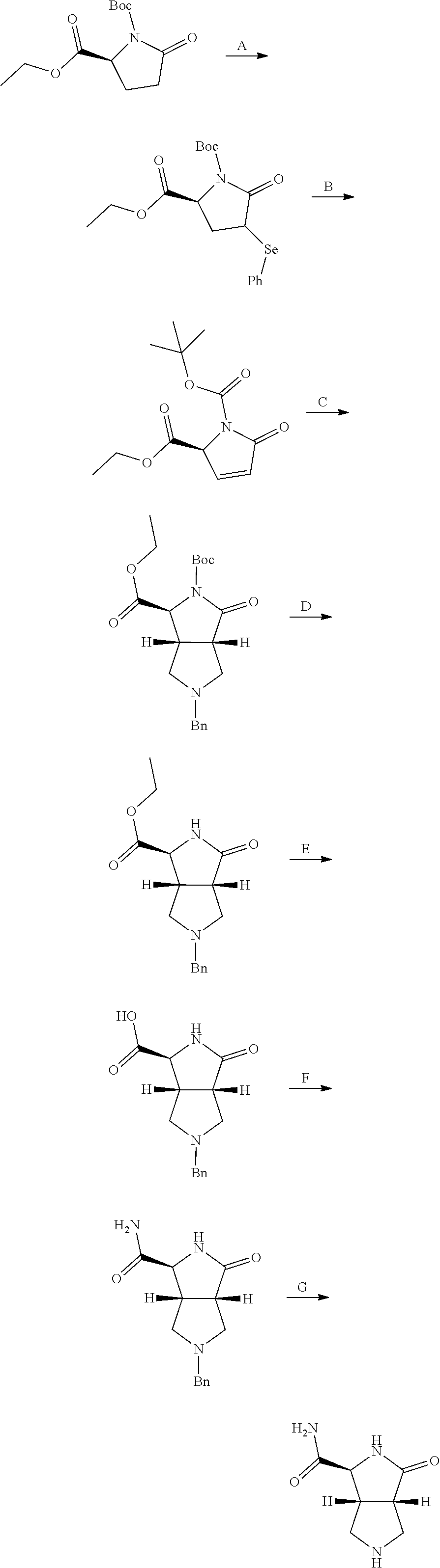

Step A. (2S)-1-tert-butyl 2-ethyl 5-oxo-4-(phenylselanyl)pyrrolidine-1,2-dicarboxylate: To a solution of(S)-1-tert-butyl 2-ethyl 5-oxopyrrolidine-1,2-dicarboxylate (2.00 g, 1.0 equiv) in THF (40 mL) was added LiHMDS (1 M, 8.55 mL, 1.1 equiv) dropwise at −78° C. The mixture was stirred at −78° C. for 1 hour under N₂ atmosphere. Then a solution of phenyl hypobromoselenoite (2.20 g, 1.2 equiv) in THF (10 mL) was added dropwise to the mixture at −78° C. The mixture was stirred at −78° C. for 2 hours under N₂ atmosphere. The reaction mixture was quenched with ammonium chloride saturated aqueous solution (50 mL) at −78° C. and extracted with ethyl acetate (20 mL×3). The combined organic layers were washed with brine (50 mL), dried over anhydrous sodium sulfate, filtered and concentrated in vacuo. The residue was purified by reversed-phase MPLC [C18, 0.1% formic acid condition] to afford the title compound (0.80 g, 25% yield) as a yellow oil. 1H NMR (400 MHZ, CHLOROFORM-d) δ=7.64 (dd, J=1.2, 8.0 Hz, 2H), 7.35-7.25 (m, 3H), 4.22 (dd, J=5.0, 8.6 Hz, 1H), 4.15 (d, J=7.2 Hz, 2H), 3.98 (dd, J=8.0, 8.6 Hz, 1H), 2.38 (dd, J=7.2, 8.2 Hz, 2H), 1.45-1.43 (m, 9H), 1.22 (t, J=7.2 Hz, 4H).

Step B: (S)-1-tert-butyl 2-ethyl 5-oxo-1H-pyrrole-1,2(2H,5H)-dicarboxylate: To a solution of (2S)-1-tert-butyl 2-ethyl 5-oxo-4-(phenylselanyl)pyrrolidine-1,2-dicarboxylate (2.00 g, 1.0 equiv) in THF (40 mL) was added H2O2 (1.15 g, 30% purity, 2.1 equiv) slowly dropwise. The resulting mixture was stirred at 35° C. for 2 hours. The reaction mixture was quenched with sodium bicarbonate saturated aqueous solution (50 mL) and extracted with ethyl acetate (50 mL×3). The combined organic layers were washed with brine (50 mL), dried over anhydrous sodium sulfate, filtered and concentrated in vacuo. The residue was purified by reversed-phase MPLC [C18, 0.1% formic acid condition] to afford the title compound (720 mg, 58% yield) as a brown oil. 1H NMR (400 MHZ, CHLOROFORM-d) δ=7.09 (dd, J=2.4, 6.0 Hz, 1H), 6.24 (dd, J=2.0, 6.0 Hz, 1H), 5.16 (t, J=2.2 Hz, 1H), 4.26 (dq, J=1.2, 7.2 Hz, 2H), 1.53 (s, 9H), 1.31 (t, J=7.2 Hz, 3H).

Step C. (1S,3aS,6aR)-2-tert-butyl 1-ethyl 5-benzyl-3-oxohexahydropyrrolo[3,4-c]pyrrole-1,2(1H)-dicarboxylate: To a solution of (S)-1-tert-butyl 2-ethyl 5-oxo-1H-pyrrole-1,2(2H,5H)-dicarboxylate (450 mg, 1.0 equiv) in DCM (3 mL) was added a solution of TFA (80.4 mg, 0.4 equiv) DCM in (5 mL). Then N-(methoxymethyl)-1-phenyl-N-(trimethylsilylmethyl) methanamine (1.26 g, 3.0 equiv) in DCM (2 mL) was added dropwise to the mixture at 0° C. The resulting mixture was allowed to warm to 25° C. and stirred at 25° C. for 3 hours. The reaction mixture was quenched with sodium bicarbonate saturated aqueous solution (50 mL) and extracted with ethyl acetate (50 mL×3). The combined organic layers were washed with brine (50 mL), dried over anhydrous sodium sulfate, filtered and concentrated in vacuo. The residue was purified by reversed-phase MPLC [C18, 0.1% formic acid condition] to afford the title compound (460 mg, 67% yield). 1H NMR (400 MHZ, METHANOL-d4) δ=7.34-7.23 (m, 5H), 4.37 (d, J=2.4 Hz, 1H), 4.29-4.19 (m, 2H), 3.69-3.52 (m, 2H), 3.18-3.08 (m, 2H), 2.94 (br d, J=10.0 Hz, 1H), 2.80-2.71 (m, 1H), 2.60 (dd, J=7.8, 9.6 Hz, 1H), 2.48-2.38 (m, 1H), 1.48 (s, 9H), 1.29 (t, J=7.2 Hz, 3H).

Step D. (1S,3aS,6aR)-ethyl 5-benzyl-3-oxooctahydropyrrolo[3,4-c]pyrrole-1-carboxylate: To a solution of (1S,3aS,6aR)-2-tert-butyl 1-ethyl 5-benzyl-3-oxohexahydropyrrolo[3,4-c]pyrrole-1,2(1H)-dicarboxylate (300 mg, 1.0 equiv) in EtOAc (2 mL) was added HCl/dioxane (4 M, 10 mL, 51.8 equiv). The mixture was stirred at 25° C. for 1 hour. The reaction mixture was concentrated under reduced pressure to afford the title compound (220 mg, 99% yield) as a white solid; LCMS (ESI, M+1): m/z=289.0.

Step E. (1S,3aS,6aR)-5-benzyl-3-oxooctahydropyrrolo[3,4-c]pyrrole-1-carboxylic acid: To a solution of (1S,3aS,6aR)-ethyl 5-benzyl-3-oxooctahydropyrrolo[3,4-c]pyrrole-1-carboxylate (220 mg, 1.0 equiv) in MeOH (9 mL) was added NaOH aqueous solution (1 M, 3.05 mL, 4.0 equiv). The mixture was stirred at 40° C. for 1 hour. The reaction mixture was poured into water (10 mL) and then pH of the mixture was adjusted to 5 with formic acid. The mixture was filtered and concentrated under reduced pressure. The residue was purified by prep-HPLC [C18, 0.1% formic acid condition] to afford the title compound (110 mg, 55% yield) as a white solid. 1H NMR (400 MHZ, METHANOL-d4) δ=7.45-7.37 (m, 5H), 4.06 (s, 2H), 3.89 (d, J=2.2 Hz, 1H), 3.07 (s, 6H); LCMS (ESI, M+1): m/z=261.0.

Step F. (1S,3aS,6aR)-5-benzyl-3-oxooctahydropyrrolo[3,4-c]pyrrole-1-carboxamide: To a mixture of (1S,3aS,6aR)-5-benzyl-3-oxooctahydropyrrolo[3,4-c]pyrrole-1-carboxylic acid (90.0 mg, 1.0 equiv, FA salt) and NH₄Cl (47.2 mg, 3.0 equiv) in DMAc (1 mL) was added PYBROP (164 mg, 1.2 equiv) and TEA (89.2 mg, 3.0 equiv), then the mixture was stirred at 40° C. for 12 hours under N₂ atmosphere. MeOH (1 mL) was added into the mixture. The mixture was directly purified by prep-HPLC [C18, 0.1% NH3·H2O condition] to afford the title compound (21.0 mg, 28% yield). 1H NMR (400 MHZ, CHLOROFORM-d) δ=7.53-7.28 (m, 5H), 6.56 (br s, 1H), 6.29 (br s, 1H), 5.75-5.48 (m, 1H), 3.88 (s, 1H), 3.70 (br d, J=13.0 Hz, 1H), 3.64-3.48 (m, 1H), 3.22 (br d, J=9.2 Hz, 1H), 3.06-2.86 (m, 3H), 2.61-2.38 (m, 2H).

Step G. (1S,3aS,6aR)-3-oxooctahydropyrrolo[3,4-c]pyrrole-1-carboxamide: To a solution of (1S,3aS,6aR)-5-benzyl-3-oxooctahydropyrrolo[3,4-c]pyrrole-1-carboxamide (21.0 mg, 1.0 equiv) in MeOH (0.5 mL) was added Pd/C (10.0 mg, 10% purity) under N₂ atmosphere. The suspension was degassed under vacuum and purged with H₂ several times. The mixture was stirred at 40° C. for 12 hours under H2 (15 Psi) atmosphere. The reaction mixture was filtered and concentrated under reduced pressure to afford the tittle compound (9.79 mg, crude), which was used directly in next step.

### Intermediate 13B

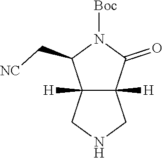 [SMILES: CC(C)(C)OC(=O)N1C(=O)[C@@H]2CNC[C@@H]2[C@H]1CC#N]

### (1R,3aS,6aR)-tert-butyl 1-(cyanomethyl)-3-oxohexahydropyrrolo[3,4-c]pyrrole-2(1H)-carboxylate

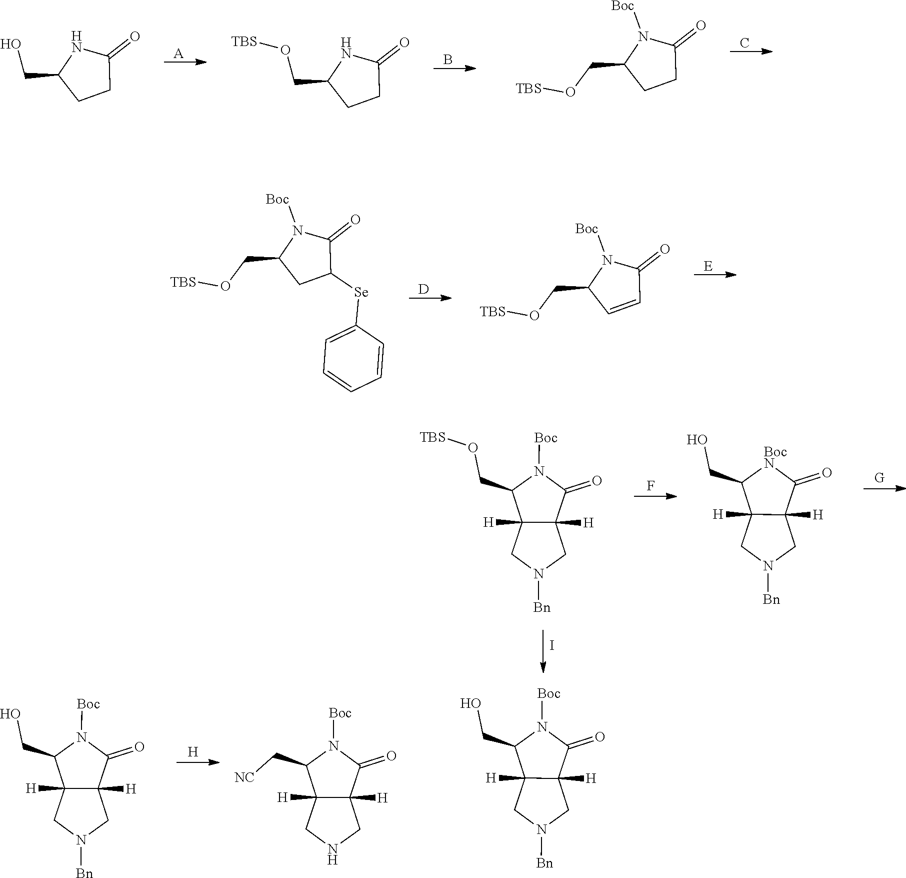

Step A. (S)-5-(((tert-butyldimethylsilyl)oxy)methyl)pyrrolidin-2-one: To a mixture of (5S)-5-(hydroxymethyl)pyrrolidin-2-one (10.0 g, 1.0 equiv) and imidazole (8.80 g, 1.5 equiv) in dichloromethane (100 mL) was added tert-butyldimethylsilyl chloride (15.7 g, 1.2 equiv) portionwise at 0-5° C. After completing the addition, the resulting mixture was allowed to warm to 25-30° C. and stirred for 12 hours. The mixture was diluted with water (150 mL) and separated. The aqueous phase was extracted with dichloromethane (2×50 mL). The combined organic layers were washed with brine, dried over sodium sulfate, filtered, and concentrated in vacuum. The residue was purified by flash silica gel chromatography [50-100% Ethyl acetate/Petroleum ether] to afford the title compound (17.5 g, 83% yield) as a colorless liquid; 1H NMR (400 MHZ, CDCl3-d4) δ=5.98 (br s, 1H), 3.81-3.71 (m, 1H), 3.62 (dd, J=4.0, 10.0 Hz, 1H), 3.44 (dd, J=7.6, 10.0 Hz, 1H), 2.40-2.30 (m, 2H), 2.22-2.11 (m, 1H), 1.81-1.69 (m, 1H), 0.88 (s, 9H), 0.06 (s, 6H); LCMS (ESI, M+1): m/z=230.2.

Step B. tert-butyl(S)-2-(((tert-butyldimethylsilyl)oxy)methyl)-5-oxopyrrolidine-1-carboxylate: To a solution of (5S)-5-[[tert-butyl(dimethyl)silyl]oxymethyl]pyrrolidin-2-one (2.0 g, 1.0 equiv) in dichloromethane (20 mL) was added tert-butyldicarbonate (2.8 g. 1.5 equiv), triethylamine (1.7 g, 2.0 equiv) and 4-dimethylaminopyridine (106 mg, 0.1 equiv). The resulting mixture was stirred at 25° C. for 12 hours. The mixture was concentrated in vacuum. The residue was purified by flash silica gel chromatography [ISCOR; 80 g SepaFlash® Silica Flash Column, Eluent of 20% Ethyl acetate/Petroleum ethergradient@100 mL/min] to afford the title compound (2.25 g, 78% yield) as a colorless oil; 1H NMR (400 MHZ, CDCl3-d4) δ=4.22-4.13 (m, 1H), 3.92 (dd, J=4.0, 10.4 Hz, 1H), 3.69 (dd, J=2.0, 10.4 Hz, 1H), 2.78-2.64 (m, 1H), 2.38 (ddd, J=2.0, 9.6, 17.6 Hz, 1H), 2.16-1.96 (m, 2H), 1.54 (s, 9H), 0.88 (s, 9H), 0.04 (d, J=5.2 Hz, 6H).

Step C. tert-butyl (5S)-5-(((tert-butyldimethylsilyl)oxy)methyl)-2-oxo-3-(phenylselanyl)pyrrolidine-1-carboxylate: To a solution of tert-butyl (2S)-2-[[tert-butyl(dimethyl)silyl]oxymethyl]-5-oxo-pyrrolidine-1-carboxylate (1.0 g, 1.0 equiv) in tetrahydrofuran (25 mL) was added lithium hexamethyldisilazide (1 M, 3.3 mL, 1.1 equiv) dropwise at −60° C. under nitrogen. The solution was stirred at −60° C. for 0.5 hour and then a solution of phenyl selenohypochlorite (1.0 g, 1.75 equiv) in tetrahydrofuran (5.0 mL) was added at −60° C. The resulting mixture was stirred at −60° C. for additional 1 hour and then warmed to 25° C. and stirred at 25° C. for 12 hours. The mixture was quenched by saturated ammonium chloride (40 mL) at 0-5° C. under nitrogen, and allowed to warm to 25° C. stirred for 0.5 hour. The mixture was extracted with ethyl acetate (3×25 mL). The combined organic layers were washed with brine, dried over sodium sulfate, filtered concentrated and purified with flash silica gel chromatography [10-15% Ethyl acetate/Petroleum ether] to afford the title compound (740 mg, 50% yield) as a yellow oil.

Step D. tert-butyl(S)-2-(((tert-butyldimethylsilyl)oxy)methyl)-5-oxo-2,5-dihydro-1H-pyrrole-1-carboxylate: To a solution of tert-butyl (5S)-5-[[tert-butyl(dimethyl)silyl]oxymethyl]-2-oxo-3-phenylselanyl-pyrrolidine-1-carboxylate (740 mg, 1.0 equiv) in dichloromethane (10.0 mL) was added pyridine (362 mg, 3.0 equiv) at −70° C., followed by slowly addition of hydrogen peroxide (606 mg, 30% purity, 3.5 equiv). The resulting mixture was allowed to warm to 25° C. and stirred at 25° C. for 12 hours. The mixture was diluted with water (20 mL) and extracted with dichloromethane (2×10 mL). The combined organic layers were washed with saturated sodium sulfite and brine, dried over sodium sulfate, filtered and concentrated in vacuum. The residue was purified by flash silica gel chromatography [10-15% Ethyl acetate/Petroleum] to afford the title compound (230 mg, 34% yield) as a colorless oil.

Step E. tert-butyl (1S,3aS,6aR)-5-benzyl-1-(((tert-butyldimethylsilyl)oxy)methyl)-3-oxohexahydropyrrolo[3,4-c]pyrrole-2(1H)-carboxylate: To a solution of tert-butyl (2S)-2-[[tert-butyl(dimethyl)silyl]oxymethyl]-5-oxo-2H-pyrrole-1-carboxylate (180 mg, 1.0 equiv) and N-(methoxymethyl)-1-phenyl-N-(trimethylsilylmethyl) methanamine (391 mg, 3.0 equiv) in dichloromethane (5.0 mL) was added TFA (25 mg, 0.4 equiv) at 0° C. After addition, the resulting mixture was stirred at 25° C. for 12 hours. Another batch of N-(methoxymethyl)-1-phenyl-N-(trimethylsilylmethyl) methanamine (391 mg, 3.0 equiv) was added, followed by TFA (25 mg, 0.4 equiv). The resulting mixture was stirred at 25° C. for another 12 hours. The mixture was concentrated in vacuum. The residue was purified by prep-HPLC [3_Phenomenex Luna C18 75×30 mm×3 um; A: water (HCl), B: CH3CN, B %: 39%-59% over 6 min], followed by lyophilization. The title compound (102 mg, 37% yield) was obtained as a yellow solid; LCMS (ESI, M+1): m/z=461.4.

Step F. (1S,3aS,6aR)-tert-butyl 5-benzyl-1-(hydroxymethyl)-3-oxohexahydropyrrolo[3,4-c]pyrrole-2(1H)-carboxylate: A mixture of (1S,3aS,6aR)-tert-butyl 5-benzyl-1-(((tert-butyldimethylsilyl)oxy)methyl)-3-oxohexahydropyrrolo[3,4-c]pyrrole-2(1H)-carboxylate (10 g, 1.0 equiv) and 4-methylbenzenesulfonic acid; hydrate (4.54 g, 1.1 uiv eq) in THF (120 mL) was stirred at 30° C. for 16 hours. The mixture was concentrated and purified by Prep-HPLC (column: Kromasil Eternity XT 250*80 mm*10 um; mobile phase: [water (ammonia hydroxide v/v)-ACN]; B %: 30%-60%, 16 min) to afford the title compound (6.2 g, 80% yield) as white solid; LCMS (ESI, M+1): m/z=347.1.

Step G. tert-butyl (1R,3aS,6aR)-5-benzyl-1-(cyanomethyl)-3-oxohexahydropyrrolo[3,4-c]pyrrole-2(1H)-carboxylate: To a solution of tert-butyl (3aS,6S,6aR)-2-benzyl-6-(hydroxymethyl)-4-oxo-3,3a,6,6a-tetrahydro-1H-pyrrolo[3,4-c]pyrrole-5-carboxylate (1.00 g, 1.0 equiv) in THF (10 mL) were added 2-hydroxy-2-methyl-propanenitrile (737 mg, 1.0 equiv) and tributylphosphine (2.04 g, 3.5 equiv). Then ADDP (2.55 g, 3.5 equiv) was added to the mixture at 0° C. The mixture was stirred at 50° C. for 2 hours. The reaction mixture was poured into saturated NH₄Cl aqueous solution (50 mL) and extracted with EtOAc (20 mL×3). The combined organic layers were washed with brine (50 mL), dried over anhydrous sodium sulfate and concentrated. The residue was purified by flash silica gel chromatography (0˜60% ethyl acetate/petroleum ether) and concentrated under vacuum to afford the title compound (600 mg, 58% yield) as a yellow solid; ¹H NMR (400 MHZ, CHLOROFORM-d) δ=7.32-7.23 (m, 5H), 7.13-7.13 (m, 1H), 4.07 (ddd, J=2.1, 4.0, 6.1 Hz, 1H), 3.67 (d, J=13.2 Hz, 1H), 3.50 (d, J=13.2 Hz, 1H), 3.25-3.18 (m, 2H), 2.90-2.80 (m, 2H), 2.77 (dd, J=2.0, 9.2 Hz, 1H), 2.64 (br d, J=8.8 Hz, 1H), 2.61-2.52 (m, 2H), 1.57 (s, 9H)

Step H. (1R,3aS,6aR)-tert-butyl 1-(cyanomethyl)-3-oxohexahydropyrrolo[3,4-c]pyrrole-2(1H)-carboxylate: To a solution of (1R,3aS,6aR)-tert-butyl 5-benzyl-1-(cyanomethyl)-3-oxohexahydropyrrolo[3,4-c]pyrrole-2(1H)-carboxylate (70.0 mg, 1.0 equiv) in MeOH (2 mL) was added Pd/C (50.0 mg, 5% purity, wet). The suspension was degassed and purged with H₂ for three times. The mixture was stirred under hydrogen atmosphere (15 Psi) at 25° C. for 16 hours. The reaction mixture was filtered and concentrated to afford the title compound (50.0 mg, 96% yield) as a yellow oil;

Step I. (1S,3aS,6aR)-tert-butyl 5-benzyl-1-(hydroxymethyl)-3-oxohexahydropyrrolo[3,4-c]pyrrole-2(1H)-carboxylate: To a solution of (1S,3aS,6aR)-tert-butyl 5-benzyl-1-(((tert-butyldimethylsilyl)oxy)methyl)-3-oxohexahydropyrrolo[3,4-c]pyrrole-2(1H)-carboxylate (10.0 g, 1.0 equiv) in THF (120 mL) was added 4-methylbenzenesulfonic acid; hydrate (4.54 g, 1.1 equiv). The mixture was stirred at 45° C. for 5 hours. The mixture was concentrated and purified with prep-HPLC [column: Kromasil Eternity XT 250×80 mm×10 um; mobile phase: water (ammonia hydroxide v/v)-CAN; B %: 30%-60%, 16 min] to afford the title compound as a white solid (6.20 g, 80% yield); 1H NMR (400 MHZ, CHLOROFORM-d) δ=7.27 (br s, 5H), 3.96 (br s, 1H), 3.84 (dd, J=3.6, 11.2 Hz, 1H), 3.73-3.61 (m, 2H), 3.56-3.48 (m, 1H), 3.18-3.11 (m, 1H), 3.07 (dd, J=2.4, 9.6 Hz, 1H), 2.65-2.59 (m, 2H), 1.55 (s, 9H); LCMS (ESI, M+1): m/z=347.2.

### Intermediate 13C

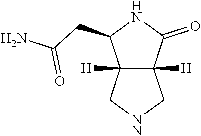

### 2-((1R,3aS,6aR)-3-oxooctahydropyrrolo[3,4-c]pyrrol-1-yl)acetamide

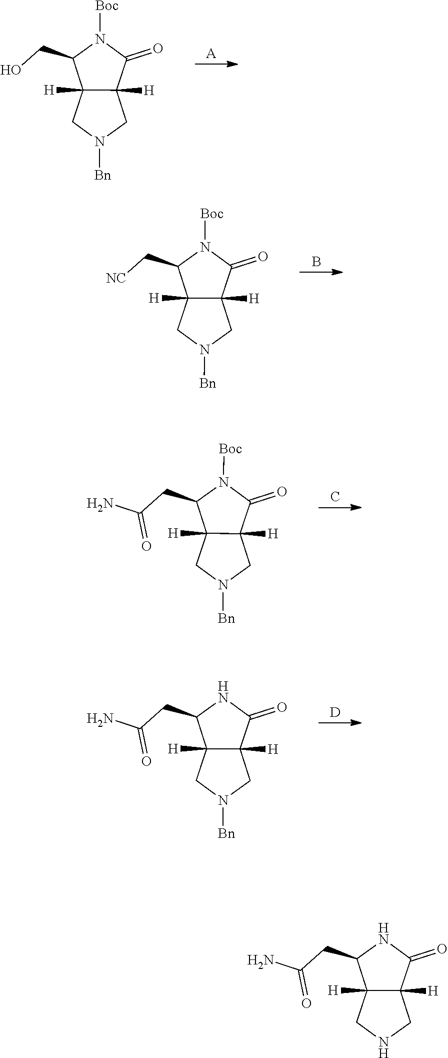

Step A. tert-butyl (1R,3aS,6aR)-5-benzyl-1-(cyanomethyl)-3-oxohexahydropyrrolo[3,4-c]pyrrole-2(1H)-carboxylate: To a solution of tert-butyl (3aS,6S,6aR)-2-benzyl-6-(hydroxymethyl)-4-oxo-3,3a,6,6a-tetrahydro-1H-pyrrolo[3,4-c]pyrrole-5-carboxylate (1.00 g, 1.0 equiv) in THF (10 mL) were added 2-hydroxy-2-methyl-propanenitrile (737 mg, 1.0 equiv) and tributylphosphane (2.04 g, 3.5 equiv). Then ADDP (2.55 g, 3.5 equiv) was added to the mixture at 0° C. The mixture was stirred at 50° C. for 2 hours. The reaction mixture was poured into saturated NH₄Cl aqueous solution (50 mL) and extracted with EtOAc (20 mL×3). The combined organic layers were washed with brine (50 mL), dried over anhydrous sodium sulfate and concentrated. The residue was purified by flash silica gel chromatography (0˜60% ethyl acetate/petroleum ether) and concentrated under vacuum to afford the title compound (600 mg, 58% yield) as a yellow solid; ¹H NMR (400 MHZ, CHLOROFORM-d) δ=7.32-7.23 (m, 5H), 7.13-7.13 (m, 1H), 4.07 (ddd, J=2.1, 4.0, 6.1 Hz, 1H), 3.67 (d, J=13.2 Hz, 1H), 3.50 (d, J=13.2 Hz, 1H), 3.25-3.18 (m, 2H), 2.90-2.80 (m, 2H), 2.77 (dd, J=2.0, 9.2 Hz, 1H), 2.64 (br d, J=8.8 Hz, 1H), 2.61-2.52 (m, 2H), 1.57 (s, 9H)

Step B. tert-butyl (1R,3aS,6aR)-1-(2-amino-2-oxoethyl)-5-benzyl-3-oxohexahydropyrrolo[3,4-c]pyrrole-2(1H)-carboxylate: To a solution of tert-butyl (3aS,6aR)-2-benzyl-6-(cyanomethyl)-4-oxo-3,3a,6,6a-tetrahydro-1H-pyrrolo[3,4-c]pyrrole-5-carboxylate (500 mg, 79.7% purity, 1.0 equiv) in EtOH (9 mL) and H₂O (1 mL) was added dimethylphosphinite dimethylphosphinous acid platinum (2+) (479 mg, 1.0 equiv). The mixture was stirred at 60° C. for 2 hours. The reaction mixture was filtered, concentrated and purified by reversed phase flash chromatography (C18, 0.1% ammonium hydroxide) to afford the title compound (420 mg, 90% yield) as a white solid; ¹H NMR (400 MHZ, METHANOL-d₄) δ=7.37-7.28 (m, 5H), 4.23 (dd, J=2.8, 8.0 Hz, 1H), 3.67-3.53 (m, 2H), 3.29-3.23 (m, 1H), 2.95 (br d, J=9.6 Hz, 1H), 2.82-2.56 (m, 6H), 1.52 (s, 9H)

Step C. 2-((1R,3aS,6aR)-5-benzyl-3-oxooctahydropyrrolo[3,4-c]pyrrol-1-yl)acetamide: To a solution of tert-butyl (3aS,6aR)-6-(2-amino-2-oxo-ethyl)-2-benzyl-4-oxo-3,3a,6,6a-tetrahydro-1H-pyrrolo[3,4-c]pyrrole-5-carboxylate (100 mg, 90.2% purity, 1.0 equiv) in MeOH (1 mL) was added HCl/dioxane (4 M, 3 mL, 49.7 equiv). The mixture was stirred at 20° C. for 0.5 hour. The reaction mixture was concentrated under reduced pressure to afford the title compound (70.0 mg, 93% yield, HCl) as a white solid.

Step D. 2-((1R,3aS,6aR)-3-oxooctahydropyrrolo[3,4-c]pyrrol-1-yl)acetamide: To a solution of 2-((1R,3aS,6aR)-5-benzyl-3-oxooctahydropyrrolo[3,4-c]pyrrol-1-yl)acetamide (50.0 mg, 1.0 equiv, HCl) in MeOH (2 mL) was added Pd/C (50.0 mg, 5% purity, wet). The suspension was degassed and purged with H₂ for three times. The mixture was stirred under H₂ atmosphere (15 Psi) at 25° C. for 2 hours. The reaction mixture was filtered and concentrated to afford the title compound (40.0 mg, crude, HCl) as a white solid.

### Intermediate 13D

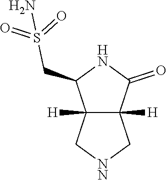

### 1-[(3aS,6S,6aR)-4-oxo-2,3,3a,5,6,6a-hexahydro-1H-pyrrolo[3,4-c]pyrrol-6-yl]methanesulfonamide

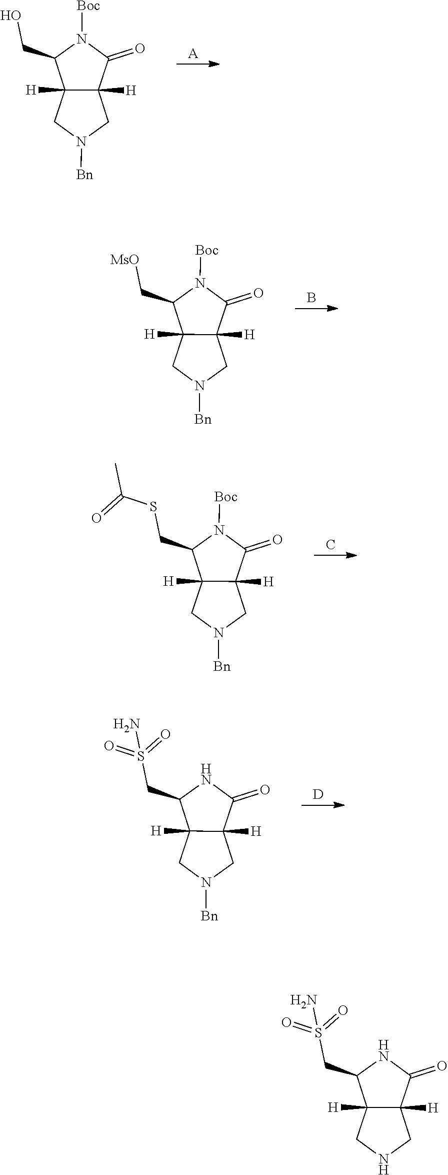

Step A. tert-butyl (3aS,6S,6aR)-2-benzyl-6-(methylsulfonyloxymethyl)-4-oxo-3,3a,6,6a-tetrahydro-1H-pyrrolo[3,4-c]pyrrole-5-carboxylate: To a solution of tert-butyl (3aS,6S,6aR)-2-benzyl-6-(hydroxymethyl)-4-oxo-3,3a,6,6a-tetrahydro-1H-pyrrolo[3,4-c]pyrrole-5-carboxylate (1.20 g, 1.0 equiv) and Ms₂O (1.81 g, 3.0 equiv) in DCM (10 mL) was added TEA (1.75 g, 5.0 equiv) and DMAP (4.23 mg, 0.01 equiv) at 0° C. The mixture was stirred at 25° C. for 2 hours. The mixture was diluted with H₂O (20 mL) and was extracted with DCM (25 mL×2). The combined organic layers were washed with brine (10 mL), dried over anhydrous sodium sulfate and concentrated. The residue was purified by flash silica gel chromatography (10˜30% EtOAc/PE) to afford the title compound (1.30 g, 79% yield) as a yellow oil; LCMS (ESI, M+1): m/z=425.2

Step B. tert-butyl (3aS,6S,6aR)-6-(acetylsulfanylmethyl)-2-benzyl-4-oxo-3,3a,6,6a-tetrahydro-1H-pyrrolo[3,4-c]pyrrole-5-carboxylate: To a solution of tert-butyl (3aS,6S,6aR)-2-benzyl-6-(methylsulfonyloxymethyl)-4-oxo-3,3a,6,6a-tetrahydro-1H-pyrrolo[3,4-c]pyrrole-5-carboxylate (1.30 g, 1.0 equiv) and TEA (1.55 g, 5.0 equiv) in DMF (10 mL) was added ethanethioic S-acid (699 mg, 3.0 equiv) at 0° C. The mixture was stirred at 25° C. for 2 hours. The mixture was diluted with H₂O (20 mL) and extracted with EtOAc (25 mL×2). The combined organic layers were washed with brine (10 mL), dried over anhydrous sodium sulfate and concentrated. The residue was purified by flash silica gel chromatography (10˜30% EtOAc/PE) to afford the title compound (1.10 g, 88% yield) as a yellow oil; LCMS (ESI, M+1): m/z=405.2

Step C. 1-[(1S,3aS,6aR)-5-benzyl-3-oxo-1,2,3a,4,6,6a-hexahydropyrrolo[3,4-c]pyrrol-1-yl]methanesulfonamide); To a solution of NCS (495 mg, 5.0 equiv) in ACN (18 mL) and HCl (2 M, 0.5 mL, 1.5 equiv) was added dropwise a solution of tert-butyl (3aS,6S,6aR)-6-(acetylsulfanylmethyl)-2-benzyl-4-oxo-3,3a,6,6a-tetrahydro-1H-pyrrolo[3,4-c]pyrrole-5-carboxylate (300 mg, 1.0 equiv) in ACN (12 mL) at 0° C. The mixture was stirred at 25° C. for 1 hour. Then NH₃·H₂O (18 mL) was added and the resulting mixture was stirred at 25° C. for 12 hours. The mixture was concentrated. The crude was purified by prep-HPLC [Phenomenex Synergi Polar-RP 100×25 mm×4 um; A: water (TFA), B: ACN, B %: 18%-38% over 7 min] and lyophilized to afford the title compound (100 mg, 32% yield, TFA) as a white solid; LCMS (ESI, M+1): m/z=310.1

Step D. 1-[(3aS,6S,6aR)-4-oxo-2,3,3a,5,6,6a-hexahydro-1H-pyrrolo[3,4-c]pyrrol-6-yl]methanesulfonamide: To a solution of 1-[(1S,3aS,6aR)-5-benzyl-3-oxo-1,2,3a,4,6,6a-hexahydropyrrolo[3,4-c]pyrrol-1-yl]methanesulfonamide (100 mg, 1.0 equiv, TFA) in MeOH (2 mL) was added Pd/C (50.0 mg, 10% purity, wet). The mixture was stirred at 25° C. under H₂ atmosphere for 2 hours. The mixture was filtered to remove the insoluble material. The filtrate was concentrated to afford the title compound (70.0 mg, 89% yield, TFA) as a white solid; LCMS (ESI, M+1): m/z=220.1.

### Intermediate 14

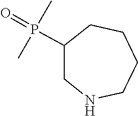

### azepan-3-yldimethylphosphine oxide

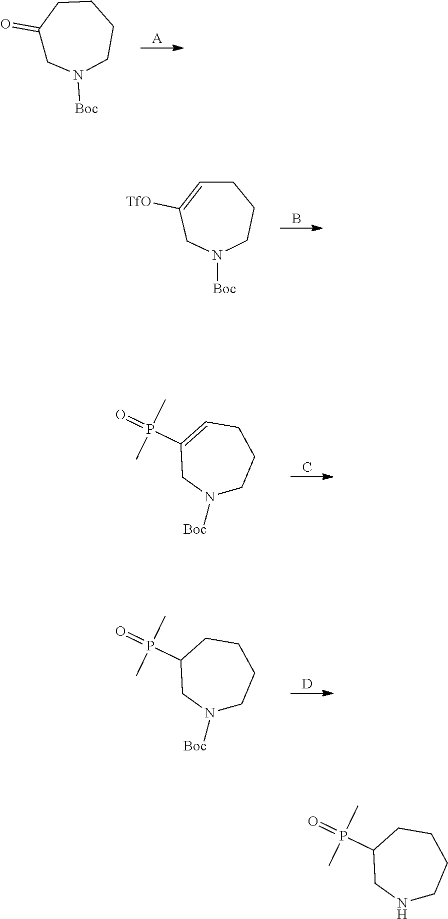

Step A. tert-butyl 6-(((trifluoromethyl) sulfonyl)oxy)-2,3,4,7-tetrahydro-1H-azepine-1-carboxylate: To a solution of tert-butyl 3-oxoazepane-1-carboxylate (5 g, 1.0 equiv) in THF (15 mL) was slowly added LiHMDS (1.0 M, 28.1 mL, 1.2 equiv) at −78° C., and the resulting mixture was stirred at −78° C. for 1 hour. The reaction mixture was concentrated and purified by column chromatography (SiO₂, petroleum ether/ethyl acetate=1/0 to 10/1) to afford the title compound (6.6 g, 78% yield) as a yellow oil. ¹H NMR (400 MHZ, CHLOROFORM-d) δ=7.14-6.79 (m, 1H), 3.70 (br t, J=5.6 Hz, 2H), 2.65-2.55 (m, 2H), 1.92-1.75 (m, 4H), 1.50 (s, 9H).

Step B. tert-butyl 6-(dimethylphosphoryl)-2,3,4,7-tetrahydro-1H-azepine-1-carboxylate: A mixture of tert-butyl 6-(((trifluoromethyl) sulfonyl)oxy)-2,3,4,7-tetrahydro-1H-azepine-1-carboxylate (500 mg, 1.0 equiv), methylphosphonoylmethane (136 mg, 1.2 equiv), TEA (220 mg, 302 μL, 1.5 equiv) and Pd(PPh₃)₄ (50.2 mg, 0.03 equiv) in ACN (5 mL) was degassed and purged with N₂ for 3 times, and then the mixture was stirred at 90° C. for 3 hours under N₂ atmosphere. The reaction mixture was filtered. The filtrate was concentrated and purified by prep-HPLC [C18, 0.1% TFA condition] to afford the title compound (180 mg, 43.1% yield, 94.8% purity) as a yellow gum. ¹H NMR (400 MHZ, CHLOROFORM-d) δ=7.37 (d, J=16.0 Hz, 1H), 3.80 (br s, 2H), 2.33 (br d, J=4.0 Hz, 2H), 1.88 (br s, 4H), 1.64 (d, J=12.8 Hz, 6H), 1.51 (s, 9H); LCMS (ESI, M+1): m/z=274.3.

Step C. tert-butyl 3-(dimethylphosphoryl)azepane-1-carboxylate: To a solution of tert-butyl 6-(dimethylphosphoryl)-2,3,4,7-tetrahydro-1H-azepine-1-carboxylate (180 mg, 1.0 equiv) in MeOH (2 mL) was added Pd/C (10%, 20 mg) under N₂ atmosphere. The suspension was degassed and purged with H₂ for 3 times. The mixture was stirred under H₂ (15 Psi) at 25° C. for 12 hours. The reaction mixture was filtered and concentrated under reduced pressure to afford the title compound (160 mg) as a yellow oil. ¹H NMR (400 MHZ, CHLOROFORM-d) δ=4.02-3.85 (m, 1H), 3.33-3.24 (m, 1H), 3.21-3.06 (m, 1H), 2.58-2.28 (m, 4H), 2.19-1.89 (m, 4H), 1.64-1.57 (m, 6H), 1.49-1.44 (m, 9H).

Step D. azepan-3-yldimethylphosphine oxide: To a solution of tert-butyl 3-(dimethylphosphoryl)azepane-1-carboxylate (160 mg, 1.0 equiv) in DCM (1.0 mL) was added HCl/dioxane (4 M, 1 mL, 6.9 equiv). The mixture was stirred at 25° C. for 2 hours. The reaction mixture was concentrated under reduce pressure to afford the title compound (120 mg, HCl salt) as a yellow oil.

### Intermediate 15

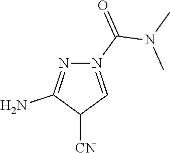

### 3-amino-4-cyano-N,N-dimethyl-1H-pyrazole-1-carboxamide

### Intermediate 15A

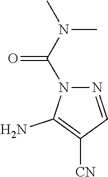

### 5-amino-4-cyano-N,N-dimethyl-1H-pyrazole-1-carboxamide

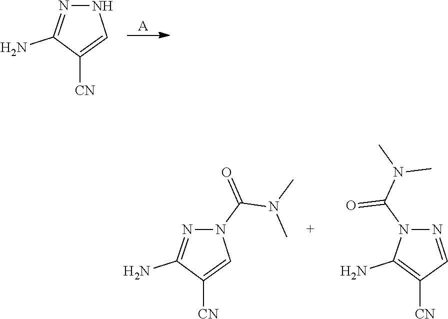

Step A. 3-amino-4-cyano-N,N-dimethyl-1H-pyrazole-1-carboxamide: To a mixture of 3-amino-1H-pyrazole-4-carbonitrile (10.0 g, 1.0 equiv), DIPEA (35.9 g, 3.0 equiv) in THF (200 mL) was added DMAP (2.26 g, 0.2 equiv) and dimethylcarbamic chloride (14.9 g, 1.5 equiv). The reaction was stirred at 60° C. for 12 hours. The mixture was quenched with water (100 ml) and extracted with EtOAc (3×100 mL). The combined organic layers were dried over anhydrous sodium sulfate, concentrated, and purified with silica gel chromatography (petroleum ether/ethyl acetate 1:0 to 0:1) to afford Intermediate 15 (5.40 g, 32% yield) as white solid; ¹H NMR (400 MHZ, dimethylsulfoxide-d₆) δ=8.62 (s, 1H), 5.99 (s, 2H), 3.07 (br s, 6H); LCMS (ESI, M−1): m/z=178.1, and Intermediate 15A (4.44 g, 26% yield) as light pink solid; ¹H NMR (400 MHZ, dimethylsulfoxide-d₆) δ=7.76 (s, 1H), 7.19 (s, 2H), 3.02 (s, 6H).

### Intermediate 16

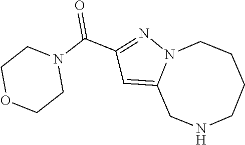 [SMILES: O=C(c1cc2n(n1)CCCCNC2)N1CCOCC1]

### (4,5,6,7,8,9-hexahydropyrazolo[1,5-a][1,4]diazocin-2-yl)(morpholino)methanone

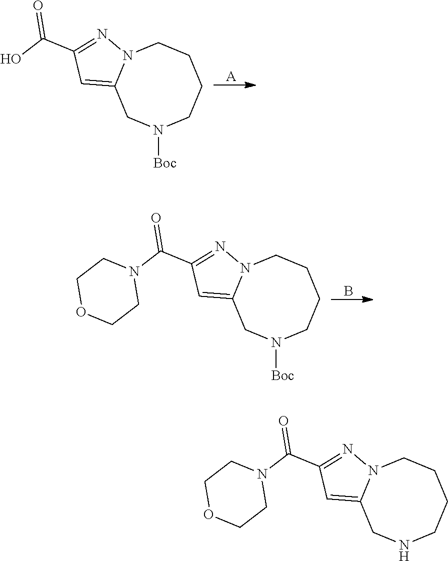

Step A: tert-butyl 2-(morpholine-4-carbonyl)-6,7,8,9-tetrahydropyrazolo[1,5-a][1,4]diazocine-5(4H)-carboxylate: To a solution of 5-tert-butoxycarbonyl-6,7,8,9-tetrahydro-4H-pyrazolo[1,5-a][1,4]diazocine-2-carboxylic acid (50.0 mg, 1.00 equiv) in DMF (1.00 mL) were added morpholine (14.8 mg, 1.00 equiv), HATU (193 mg, 3.00 equiv) and DIPEA (219 mg, 10.0 equiv). The mixture was stirred at 20° C. for 1 hour. After the reaction mixture was diluted with water (10.0 mL) and extracted with ethyl acetate (10.0 mL×2). The combined organic layers were washed with brine (10.0 mL), dried over anhydrous sodium sulfate, filtered and concentrated under reduced pressure to give a residue. The residue was purified with prep-TLC [SiO₂, PE/EA=0/1] to afford the title compound (25.0 mg, 40% yield, 99% purity) as a brown oil; ¹H NMR (400 MHZ, CDCl₃) δ=6.57 (s, 1H), 4.69-4.50 (m, 2H), 4.31 (br s, 2H), 4.05 (br d, J=18.8 Hz, 2H), 3.76 (br s, 4H), 3.70 (br s, 2H), 3.48-3.24 (m, 2H), 1.89 (br s, 2H), 1.69-1.57 (m, 2H), 1.47 (s, 9H). LCMS (ESI, M+1): m/z=365.2

Step B: (4,5,6,7,8,9-hexahydropyrazolo[1,5-a][1,4]diazocin-2-yl)(morpholino)methanone: A solution of tert-butyl 2-(morpholine-4-carbonyl)-6,7,8,9-tetrahydro-4H-pyrazolo[1,5-a][1,4]diazocine-5-carboxylate (240 mg, 1.00 equiv) in MeCN (5.00 mL) and HCl/dioxane (4 M, 5.00 mL, 30.37 equiv) was stirred at 20° C. for 1 hour. The reaction mixture was concentrated under reduced pressure to remove solvent and afford the title compound (330 mg, crude) as a colorless oil. LCMS (ESI, M+1): m/z=265.1

### Intermediate 16A

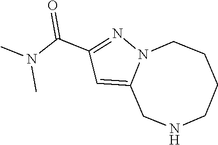 [SMILES: CN(C)C(=O)c1cc2n(n1)CCCCNC2]

### N,N-dimethyl-4,5,6,7,8,9-hexahydropyrazolo[1,5-a][1,4]diazocine-2-carboxamide

### Intermediate 16B

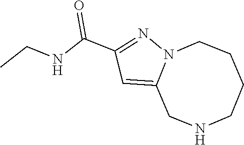 [SMILES: CCNC(=O)c1cc2n(n1)CCCCNC2]

### N-ethyl-4,5,6,7,8,9-hexahydropyrazolo[1,5-a][1,4]diazocine-2-carboxamide

Intermediates 16A and 16B were synthesized following the 2-step procedure described for Intermediate 16.

### Intermediate 17

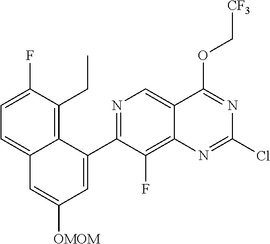

### 2-chloro-7-(8-ethyl-7-fluoro-3-(methoxymethoxy)naphthalen-1-yl)-8-fluoro-4-(2,2,2-trifluoroethoxy)pyrido[4,3-d]pyrimidine

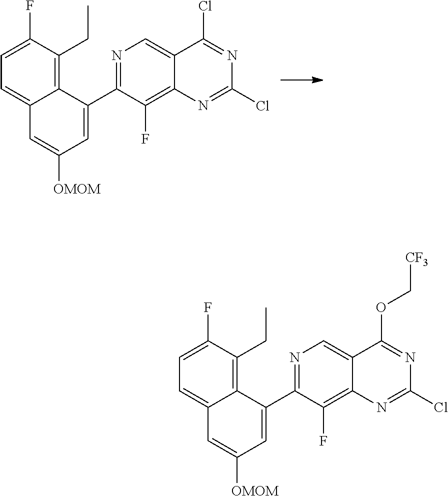

To a solution of CF₃CH₂OH (2.57 g, 1.05 equiv) in THF (50 mL) was added t-BuONa (2 M, 12.8 mL, 1.05 equiv). The mixture was stirred at 20° C. for 1 hour. To the mixture was added dropwise a solution of 2,4-dichloro-7-(8-ethyl-7-fluoro-3-(methoxymethoxy)naphthalen-1-yl)-8-fluoropyrido[4,3-d]pyrimidine (11.0 g, 1.0 equiv) in THF (100 mL) at −40° C. The reaction was stirred at −40° C. for 0.5 hour. The mixture was diluted with water (200 mL) and extracted with ethyl acetate (100 mL). The organic layer was dried over Na₂SO₄ and concentrated to afford the title compound (12.0 g, 92% yield) as a yellow solid; LCMS (ESI, M+1, M+3): m/z=514.2, 516.2.

### Intermediate 18

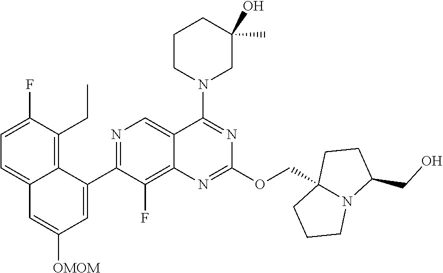

### (R)-1-(7-(8-ethyl-7-fluoro-3-(methoxymethoxy)naphthalen-1-yl)-8-fluoro-2-(((3S,7aS)-3-(hydroxymethyl)hexahydro-1H-pyrrolizin-7a-yl)methoxy)pyrido[4,3-d]pyrimidin-4-yl)-3-methylpiperidin-3-ol

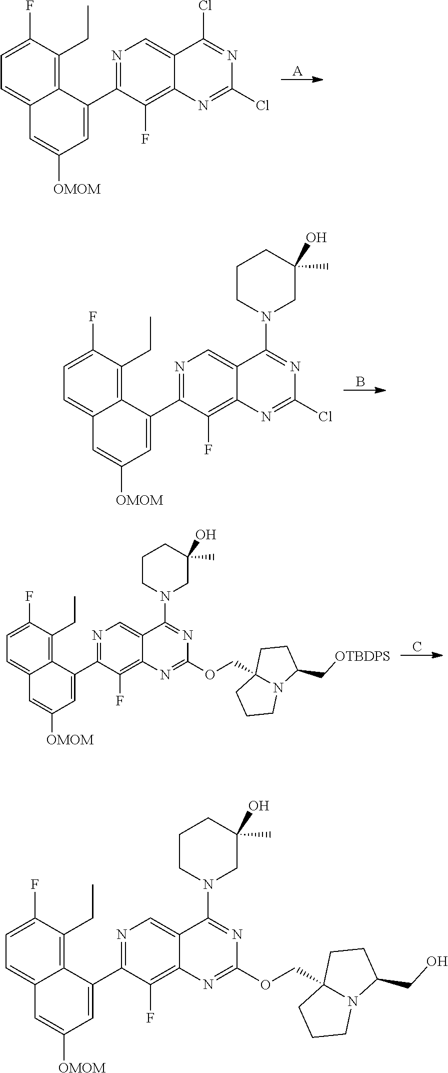

Step A. (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution 2,4-dichloro-7-[8-ethyl-7-fluoro-3-(methoxymethoxy)-1-naphthyl]-8-fluoro-pyrido[4,3-d]pyrimidine (10.0 g, 1.0 equiv) and (R)-3-methylpiperidin-3-ol (4.04 g, 1.2 equiv, HCl) in dichloromethane (200 mL) was added DIPEA (11.5 g, 4.0 equiv) and 4 Å molecular sieves (1.0 g, 1.0 equiv). The mixture was stirred at −40° C. for 15 minutes. The reaction mixture was filtered, washed with EtOAc (100 mL) and extracted with EtOAc (2×100 mL). The combined organic layers were dried over anhydrous sodium sulfate, concentrated and purified by reversed phase flash chromatography [C18, 0.1% formic acid condition] to afford the title compound (10.0 g, 84% yield) as yellow liquid; LCMS (ESI, M+1): m/z=529.2.

Step B. (R)-1-(2-(((3S,7aS)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of ((3S,7aS)-3-(((tert-butyldiphenylsilyl)oxy)methyl)tetrahydro-1H-pyrrolizin-7a(5H)-yl)methanol (4.26 g, 1.1 equiv) and 4 Å molecular sieves (150 mg, 1.0 equiv) in toluene (50 mL) was added t-BuONa (2.73 g, 3.0 equiv) at 0° C. The mixture was stirred at 0° C. for 10 minutes. Then (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (5.0 g, 1.0 equiv) was added to the mixture and the mixture was stirred at 0° C. for 1 hour. The reaction mixture was filtered, washed with EtOAc (100 mL). The filtrate was quenched with water (30 mL) and extracted with EtOAc (2×50 mL). The combined organic layers were dried over anhydrous sodium sulfate, concentrated and purified by reversed phase flash chromatography [C18, 0.1% formic acid condition] to afford the title compound (6.60 g, 76% yield) as yellow liquid; LCMS (ESI, M+1): m/z=902.6.

Step C. (R)-1-(7-(8-ethyl-7-fluoro-3-(methoxymethoxy)naphthalen-1-yl)-8-fluoro-2-(((3S,7aS)-3-(hydroxymethyl)hexahydro-1H-pyrrolizin-7a-yl)methoxy)pyrido[4,3-d]pyrimidin-4-yl)-3-methylpiperidin-3-ol: To a solution of (R)-1-(2-(((3S,7aS)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (6.60 g, 1.0 equiv) in DMF (66 mL) was added CsF (16.7 g, 15 equiv). The mixture was stirred at 40° C. for 12 hours. The reaction mixture was filtered, washed with EtOAc (30 mL). The filtrate was concentrated and purified by reversed phase flash chromatography [C18, 0.1% formic acid condition] to afford the title compound (4.0 g, 80% yield) as yellow liquid; LCMS (ESI, M+1): m/z=664.4

### Intermediate 19

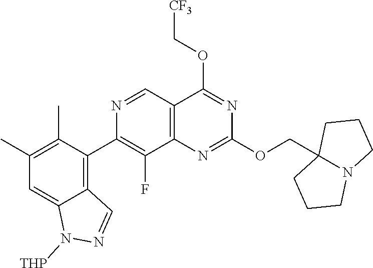

### 7-(5,6-dimethyl-1-(tetrahydro-2H-pyran-2-yl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine

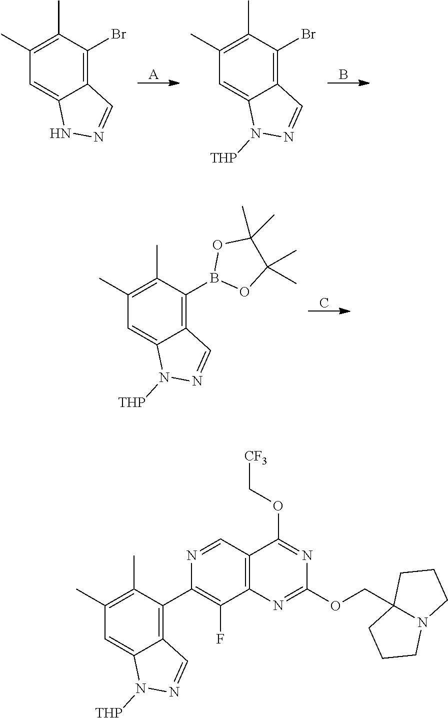

Step A. 4-bromo-5,6-dimethyl-1-(tetrahydro-2H-pyran-2-yl)-1H-indazole: To a solution of 4-bromo-5,6-dimethyl-1H-indazole (4.5 g, 1.0 equiv) and TsOH (104 mg, 0.03 equiv) in DCM (100 mL) was added 3,4-dihydro-2H-pyran (2.56 g, 1.5 equiv) dropwise at 0° C. The mixture was stirred at 25° C. for 6 hours. The mixture was diluted with H₂O (30 mL) and saturated NaHCO₃ aqueous (30 mL), extracted with DCM (3×20 mL). The combined organic layer was dried over anhydrous Na₂SO₄, concentrated and purified by column chromatography [SiO₂, petroleum ether/ethyl acetate 15:1 to 4:1] to afford the title compound (5.7 g, 91% yield) as a yellow oil; ¹H NMR (400 MHZ, methanol-d₄) δ=7.88 (s, 1H), 7.46 (s, 1H), 5.74 (dd, J=2.8, 9.6 Hz, 1H), 4.04-3.95 (m, 1H), 3.84-3.76 (m, 1H), 2.48 (s, 3H), 2.44 (s, 3H), 2.16-2.08 (m, 1H), 2.05-1.95 (m, 1H), 1.90-1.72 (m, 2H), 1.64-1.49 (m, 2H).

Step B. 5,6-dimethyl-1-(tetrahydro-2H-pyran-2-yl)-4-(4,4,5,5-tetramethyl-1,3,2-dioxaborolan-2-yl)-1H-indazole: To a mixture of 4-bromo-5,6-dimethyl-1-(tetrahydro-2H-pyran-2-yl)-1H-indazole (5.7 g, 1.0 equiv), 4,4,4′,4′,5,5,5′,5′-octamethyl-2,2′-bi (1,3,2-dioxaborolane) (7.49 g, 1.6 equiv) in DMSO (60 mL) were added AcOK (5.43 g, 3.0 equiv) and Pd(dppf)Cl₂ (809 mg, 0.06 equiv) under N₂ atmosphere. The mixture was stirred at 110° C. for 0.75 hours. The mixture was filtered through a pad of celite. The filter cake was washed with ethyl acetate (200 mL). The mixture was diluted with H₂O (200 mL) and extracted with ethyl acetate (4×30 mL). The combined organic layer was dried over anhydrous Na₂SO₄, concentrated and purified with column chromatography [SiO₂, petroleum ether/ethyl acetate 20:1 to 15:1] to afford the title compound (3.3 g, 50% yield) as a light yellow oil; ¹H NMR (400 MHZ, methanol-d₄) δ=8.21 (s, 1H), 7.53 (s, 1H), 5.72 (dd, J=2.4, 10.0 Hz, 1H), 4.04-3.95 (m, 1H), 3.80 (dt, J=2.8, 11.2 Hz, 1H), 2.54 (s, 3H), 2.52-2.44 (m, 1H), 2.42 (s, 3H), 2.16-2.06 (m, 1H), 2.00-1.92 (m, 1H), 1.87-1.61 (m, 3H), 1.42 (s, 12H).

Step C. 7-(5,6-dimethyl-1-(tetrahydro-2H-pyran-2-yl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine: To a mixture of 7-chloro-8-fluoro-2-((hexahydro-1H-pyrrolizin-7a-yl)methoxy)-4-(2,2,2-trifluoroethoxy)pyrido[4,3-d]pyrimidine (1 g, 1.0 equiv), 5,6-dimethyl-1-(tetrahydro-2H-pyran-2-yl)-4-(4,4,5,5-tetramethyl-1,3,2-dioxaborolan-2-yl)-1H-indazole (1.00 g, 1.2 equiv) in methoxycyclopentane (15 mL) were added Cs₂CO₃ (1 M in H₂O, 3.0 equiv) and CataCXium A Pd G3 (173 mg, 0.1 equiv) under N₂ atmosphere. The mixture was stirred at 80° C. for 6 hours. The mixture was diluted with H₂O (10 mL) and extracted with ethyl acetate (5×10 mL). The combined organic layer was dried over anhydrous Na₂SO₄, concentrated and purified with reversed phase flash chromatography [water (FA, 0.1%)/acetonitrile=3/1 to 2/1] to afford the title compound (0.66 g, 44% yield) as a light red solid; LCMS (ESI, M+1): m/z=615.4.

### Intermediate 20

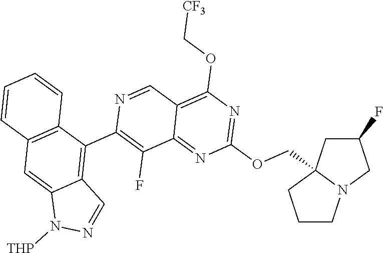

### 4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole

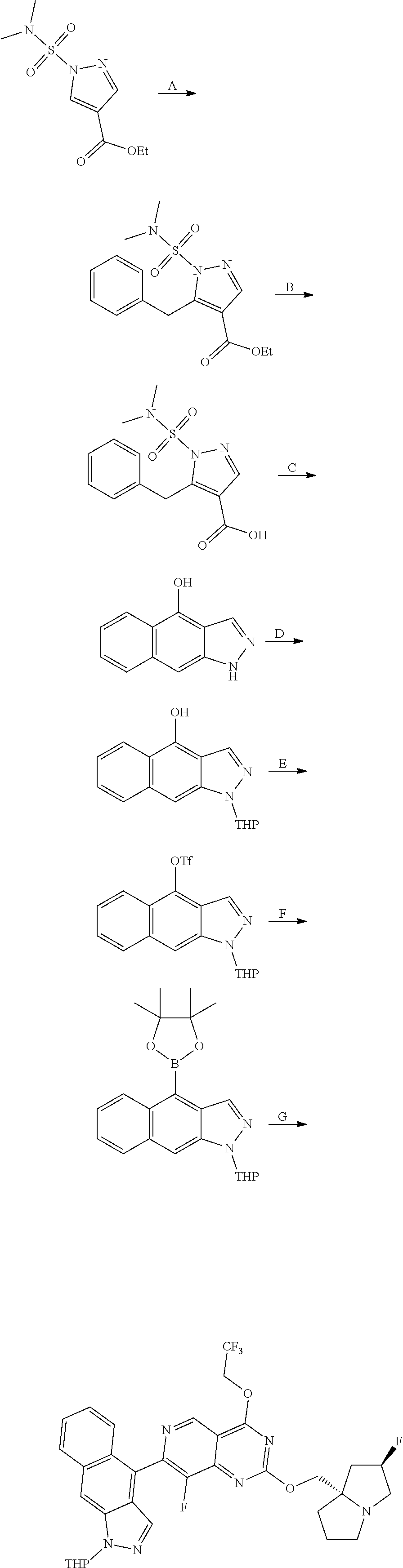

Step A. ethyl 5-benzyl-1-(N,N-dimethylsulfamoyl)-1H-pyrazole-4-carboxylate: To a mixture of ethyl 1-(dimethylsulfamoyl) pyrazole-4-carboxylate (150 g, 1.0 equiv) in THF (1000 mL) were added HMPA (130 g, 1.2 equiv) and LDA (2 M, 1.2 equiv) at −65° C. under nitrogen atmosphere. After stirring at −65° C. for 0.5 hour, bromomethylbenzene (124 g, 1.2 equiv) was added. The reaction was stirred at −65° C. for 0.5 hour under nitrogen atmosphere. The mixture was quenched with water (500 mL) and extracted with ethyl acetate (500 mL×2). The combined organic layers were washed with brine (500 mL), dried over anhydrous sodium sulfate, concentrated and purified by reversed phase flash chromatography [water (FA, 0.1%)/acetonitrile] to afford the title compound (130 g, 45% yield) as a yellow solid.

Step B. 5-benzyl-1-(N,N-dimethylsulfamoyl)-1H-pyrazole-4-carboxylic acid: A mixture of ethyl 5-benzyl-1-(dimethylsulfamoyl) pyrazole-4-carboxylate (110 g, 1.0 equiv) and NaOH (195 g, 15 equiv) in dioxane (600 mL) and H₂O (600 mL) was stirred at 25° C. for 1 hour. The mixture was extracted with ethyl acetate (2×1000 mL). The combined organic layers were dried over anhydrous sodium sulfate and concentrated to afford the title compound (95 g, crude) as a yellow solid. LCMS (ESI, M−1): m/z=310.0.

Step C. 1H-benzo[f]indazol-4-ol: A mixture of 5-benzyl-1-(dimethylsulfamoyl) pyrazole-4-carboxylic acid (30.0 g, 1.0 eq) in CF₃SO₃H (150 mL) was stirred at 90° C. for 2 hours. The mixture was poured into ice water (1000 mL) and extracted with ethyl acetate (2×1000 mL). The combined organic layers were dried over anhydrous sodium sulfate and concentrated to afford the title compound (43.5 g, crude) as a yellow solid. LCMS (ESI, M+1): m/z=184.9.

Step D. 1-(tetrahydro-2H-pyran-2-yl)-1H-benzo[f]indazol-4-ol: To a mixture of 1H-benzo[f]indazol-4-ol (12.0 g, 1.0 equiv) and TsOH·H₂O (123 mg, 0.01 equiv) in THF (120 mL) was added DHP (10.9 g, 2.0 equiv). The reaction was stirred at 20° C. for 1 hours. The mixture was diluted with water (300 mL) and extracted with ethyl acetate (2×500 mL). The combined organic layers were dried over anhydrous sodium sulfate, concentrated and purified by reversed phase flash chromatography [water (FA, 0.1%)/acetonitrile] to afford the title compound (16 g, 74% yield) as a yellow solid.

Step E. 1-(tetrahydro-2H-pyran-2-yl)-1H-benzo[f]indazol-4-yl trifluoromethanesulfonate: To a mixture of 1-tetrahydropyran-2-ylbenzo[f]indazol-4-ol (9.2 g, 1.0 equiv), DIEA (17.3 g, 4.0 equiv) and 4 Å molecular sieves (1.00 g) in DCM (100 mL) was added trifluoromethanesulfonic anhydride (19.3 g, 2.0 equiv) at −40° C. The reaction was stirred at −40° C. for 0.5 hour. The mixture was quenched with H₂O (200 mL) and extracted with DCM (3×100 mL). The combined organic layers were washed with brine (100 mL), dried over anhydrous sodium sulfate, concentrated and purified with reversed-phase HPLC (0.1% FA condition) to afford the title compound (2.8 g, 20% yield) as a black solid;

Step F. 1-(tetrahydro-2H-pyran-2-yl)-4-(4,4,5,5-tetramethyl-1,3,2-dioxaborolan-2-yl)-1H-benzo[f]indazole: To a solution of 1-(tetrahydro-2H-pyran-2-yl)-1H-benzo[f]indazol-4-yl trifluoromethanesulfonate (2.80 g, 1.0 equiv), 4,4,5,5-tetramethyl-1,3,2-dioxaborolane (8.95 g, 10 equiv) and TEA (2.12 g, 3.0 equiv) in MeCN (30 mL) was added (1,1′-bis(diphenylphosphino)ferrocene)palladium (II) dichloride (511 mg, 0.10 equiv). The reaction was stirred at 80° C. for 5 hours. The mixture was concentrated, dissolved in water (20 mL) and extracted with ethyl acetate (3×20 mL). The combined organic layers were washed with brine (30 mL), dried over anhydrous sodium sulfate, concentrated and purified with reversed-phase HPLC (0.1% FA condition) to afford the title compound (800 mg, 30% yield) as a brown solid.

Step G. 4-(8-fluoro-2-(((2R,7aS)-2-fluorohexahydro-1H-pyrrolizin-7a-yl)methoxy)-4-(2.2.2 trifluoroethoxy)pyrido[4,3-d]pyrimidin-7-yl)-1-(tetrahydro-2H-pyran-2-yl)-1H-benzo[f]indazole: To a solution of 1-(tetrahydro-2H-pyran-2-yl)-4-(4,4,5,5-tetramethyl-1,3,2-dioxaborolan-2-yl)-1H-benzo[f]indazole (200 mg, 1.0 equiv) 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (232 mg, 1.0 equiv) and Cs₂CO₃ (517 mg, 1.5 M, 3.0 equiv) in methoxycyclopentane (2 mL) was added [2-(2-aminophenyl)phenyl]palladium bis(1-adamantyl)butylphosphane methanesulfonate (38.5 mg, 0.10 equiv). The reaction was stirred at 90° C. for 2 hours. The mixture was filtered and purified with reversed-phase HPLC (0.1% FA condition) to afford the title compound (320 g, 92% yield) as a brown solid; LCMS (ESI, M+1): m/z=655.3.

### Intermediate 21

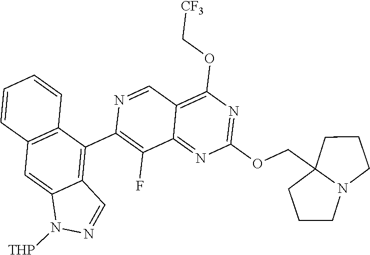

### 4-(8-fluoro-2-((hexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole

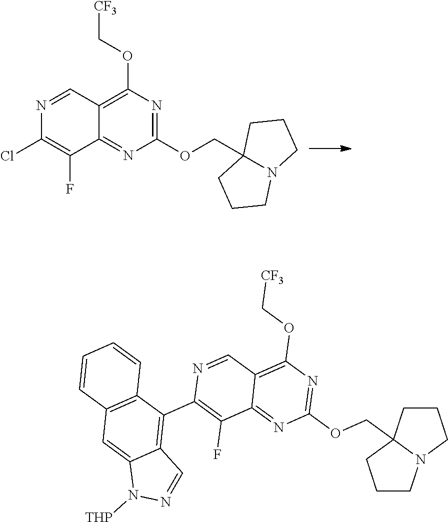

To a solution of 1-(tetrahydro-2H-pyran-2-yl)-4-(4,4,5,5-tetramethyl-1,3,2-dioxaborolan-2-yl)-1H-benzo[f]indazole (250 mg, 1.0 equiv), 7-chloro-8-fluoro-2-((tetrahydro-1H-pyrrolizin-7a(5H)-yl)methoxy)-4-(2,2,2-trifluoroethoxy)pyrido[4,3-d]pyrimidine (278 mg, 1.0 equiv) and Cs₂CO₃ (646 mg, 1.5 M, 3.0 equiv) in methoxycyclopentane (2 mL) was added [2-(2-aminophenyl)phenyl]palladium bis(1-adamantyl)butylphosphane methanesulfonate (48.1 mg, 0.10 equiv). The reaction was stirred at 90° C. for 2 hours. The mixture was filtered and purified by reversed-phase HPLC (0.1% FA condition) to afford the title compound (120 mg, 28% yield) as a yellow solid; LCMS (ESI, M+1): m/z=637.3.

### Intermediate 22

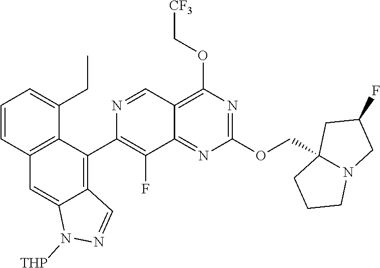

### 5-ethyl-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole

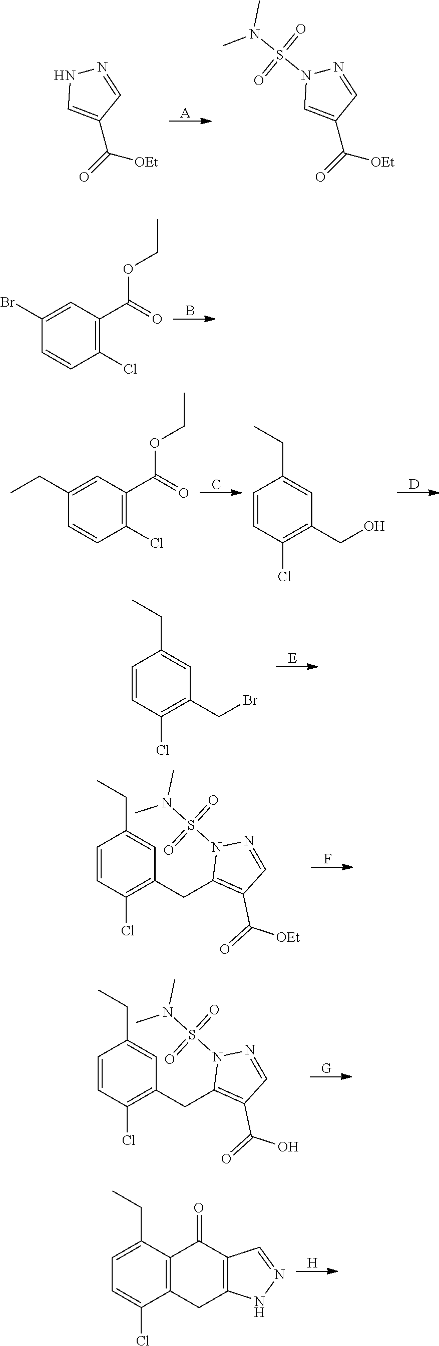

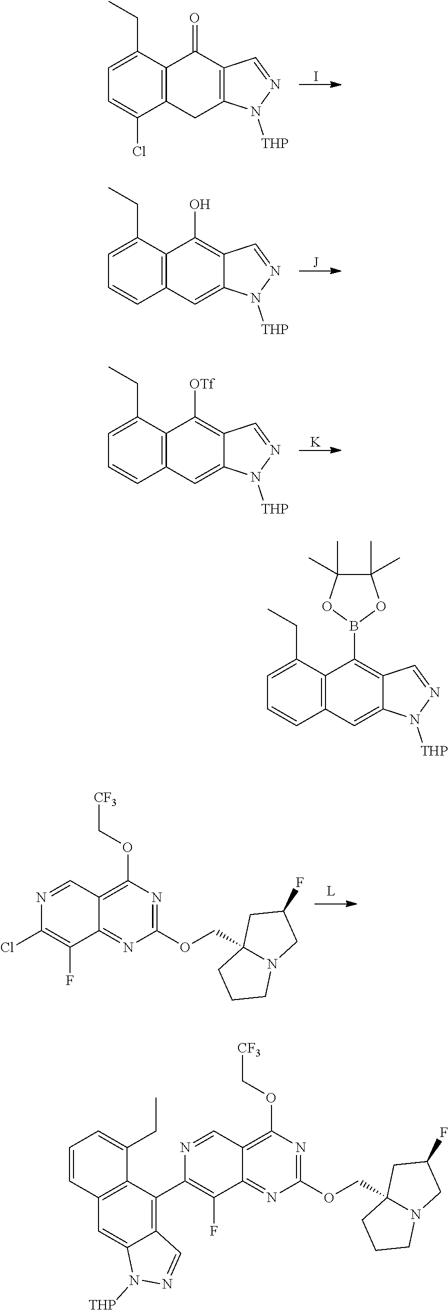

Step A. ethyl 1-(N,N-dimethylsulfamoyl)-1H-pyrazole-4-carboxylate: To a solution of ethyl 1H-pyrazole-4-carboxylate (20.0 g, 1.0 equiv) and DABCO (17.6 g, 1.1 equiv) in MeCN (200 mL) was added N,N-dimethylsulfamoyl chloride (22.5 g, 1.1 equiv). The mixture was stirred at 25° C. for 1 hour. The mixture was concentrated and purified by column chromatography [SiO₂, Petroleum ether/Ethyl acetate 8:1] to afford the title compound (29.0 g, 82% yield) as a white solid; ¹H NMR (400 MHZ, DMSO-d6) δ=8.66 (s, 1H), 8.22 (s, 1H), 4.28-4.23 (m, 2H), 2.90 (s, 6H), 1.30-1.26 (m, 3H); LCMS (ESI, M+1): m/z=248.2.

Step B. ethyl 2-chloro-5-ethylbenzoate: A mixture of ethyl 5-bromo-2-chlorobenzoate (66.0 g, 1.0 equiv), triethylborane (1 M, 501 mL, 2.0 equiv), K₂CO₃ (69.2 g, 2.0 equiv) and Pd(PPh₃)₄ (28.9 g, 0.1 equiv) in DMF (600 mL) and THF (600 mL) was degassed and purged with N₂ for 3 times. The mixture was stirred at 70° C. for 12 hours under N₂ atmosphere. The mixture was filtered and diluted with ethyl acetate (3000 mL). The organic layer was washed by brine (5×2000 mL), dried over anhydrous sodium sulfate, concentrated and purified by column chromatography [SiO₂, Petroleum ether/Ethyl acetate 10:1] to afford the title compound (98.0 g, 92% yield) as a yellow oil; ¹H NMR (400 MHZ, CHLOROFORM-d) δ=7.58 (d, J=2.0 Hz, 1H), 7.30-7.20 (m, 1H), 7.19-7.16 (m, 1H), 4.36 (dd, J=7.2, 14.4 Hz, 2H), 2.63-2.57 (m, 2H), 1.38-1.34 (m, 3H), 3.00 (s, 6H), 1.21-1.17 (m, 3H).

Step C. (2-chloro-5-ethylphenyl)methanol: To a solution of ethyl 2-chloro-5-ethylbenzoate (73.0 g, 1.0 equiv) in THF (500 mL) was added DIBAL-H (1 M, 700 mL, 2.0 equiv) at 0° C. under N₂ atmosphere. The solution was stirred at 0-25° C. for 12 hours. The mixture was quenched by ice water (1000 mL) and extracted with ethyl acetate (3×500 mL). The organic layer was dried over anhydrous sodium sulfate, concentrated and purified by column chromatography [SiO₂, Petroleum ether/Ethyl acetate 10:1] to afford the title compound (49.0 g, 82% yield) as a yellow oil; ¹H NMR (400 MHZ, CHLOROFORM-d) δ=7.32 (d, J=1.6 Hz, 1H), 7.29-7.22 (m, 1H), 7.07 (dd, J=2.0, 8.0 Hz, 1H), 4.74 (br d, J=3.6 Hz, 2H), 2.64 (q, J=7.6 Hz, 2H), 1.26-1.21 (m, 3H)

Step D. 2-(bromomethyl)-1-chloro-4-ethylbenzene: To a solution of (2-chloro-5-ethylphenyl)methanol (98.0 g, 1.0 equiv) in DCM (600 mL) was added PBr₃ (171 g, 1.1 equiv) at 0° C. slowly. The mixture was stirred at 0° C. for 2 hours. The mixture was added into saturated NaHCO₃ solution (2 L) at 0° C. and extracted with ethyl acetate (3×500 mL). The organic layer was dried over Na₂SO₄, concentrated and purified by column chromatography [SiO₂, Petroleum ether/Ethyl acetate 1:0] to afford the title compound (78.0 g, 58% yield) as a colorless oil.

Step E. ethyl 5-(2-chloro-5-ethylbenzyl)-1-(N,N-dimethylsulfamoyl)-1H-pyrazole-4-carboxylate: To a mixture of ethyl 1-(N,N-dimethylsulfamoyl)-1H-pyrazole-4-carboxylate (52.9 g, 1.0 equiv) and HMPA (46.0 g, 1.2 equiv) in THF (550 mL) was added LDA (2 M, 128 mL, 1.2 equiv) at −60° C. The mixture was stirred at −60° C. for 1 hour. To the mixture was added 2-(bromomethyl)-1-chloro-4-ethylbenzene (60.0 g, 1.2 equiv) at −60° C. The mixture was stirred at −60° C. for 1 hour and warmed to 15° C. for 12 hours. The mixture was diluted with water (300 mL) and extracted with ethyl acetate (2×300 mL). The organic layer was washed with brine (100 ml) and dried over Na₂SO₄. The organic phase was concentrated and purified by column chromatography [SiO₂, Petroleum ether/Ethyl acetate 50:1 to 8:1] to afford the title compound (35.0 g, 38% yield) as a white solid; ¹H NMR (400 MHZ, CHLOROFORM-d) δ=8.12 (s, 1H), 7.34-7.22 (m, 1H), 6.97 (dd, J=1.2, 8.0 Hz, 1H), 6.43 (s, 1H), 4.77 (s, 2H), 4.25 (q, J=7.2 Hz, 2H), 3.00 (s, 6H), 2.47 (q, J=7.6 Hz, 2H), 1.24 (t, J=7.2 Hz, 3H), 1.09 (t, J=7.6 Hz, 3H); LCMS (ESI, M+1): m/z=400.1.

Step F. 5-(2-chloro-5-ethylbenzyl)-1-(N,N-dimethylsulfamoyl)-1H-pyrazole-4-carboxylic acid: A mixture of ethyl 5-(2-chloro-5-ethylbenzyl)-1-(N,N-dimethylsulfamoyl)-1H-pyrazole-4-carboxylate (5.00 g, 1.0 equiv) and NaOH (10.0 g, 20 equiv) in dioxane (30 mL) and H₂O (30 mL) was stirred at 90° C. for 2 hours. The mixture was diluted with ethyl acetate (100 mL). The organic layer was dried over Na₂SO₄ and concentrated to to afford the title compound (6.00 g, crude) as a yellow solid; LCMS (ESI, M+1): m/z=372.1.

Step G. 8-chloro-5-ethyl-1H-benzo[f]indazol-4(9H)-one: A mixture of 5-(2-chloro-5-ethylbenzyl)-1-(N,N-dimethylsulfamoyl)-1H-pyrazole-4-carboxylic acid (6.00 g. 1.0 equiv) in CF₃SO₃H (102 g, 42 equiv) was stirred at 90° C. for 1.5 hours. The mixture was quenched by ice water (200 mL) and filtered. The filter cake was partitioned between ethyl acetate (100 mL) and sat. NaHCO₃ (100 mL). The organic layer was dried over Na₂SO₄ and concentrated to afford the title compound (3.20 g, 74 yield) as a yellow solid; LCMS (ESI, M+1): m/z=247.0.

Step H. 8-chloro-5-ethyl-1-(tetrahydro-2H-pyran-2-yl)-1H-benzo[f]indazol-4(9H)-one: To a solution of 8-chloro-5-ethyl-1H-benzo[f]indazol-4(9H)-one (12.0 g, 1.0 equiv) and TsOH (838 mg, 0.1 equiv) in THF (120 mL) was added DHP (5.32 g, 1.3 equiv) at 0° C. The mixture was stirred at 15° C. for 0.5 hour. The mixture was diluted with water (200 mL) and extracted with ethyl acetate (2×100 mL). The organic layer was dried and concentrated to give a residue. The residue was purified by column chromatography [SiO₂, Petroleum ether/Ethyl acetate 8:1 to 2:1] to afford the title compound (8.00 g, 42% yield) as a yellow solid.

Step I. 5-ethyl-1-(tetrahydro-2H-pyran-2-yl)-1H-benzo[f]indazol-4-ol: To a suspension of Pd/C (500 mg, 10% purity) and NaHCO₃ (1.02 g, 1.0 equiv) in MeOH (40 mL) was added 8-chloro-5-ethyl-1-(tetrahydro-2H-pyran-2-yl)-1H-benzo[f]indazol-4(9H)-one (4.00 g, 1.0 equiv) under N₂. The suspension was degassed under vacuum and purged with H₂ several times. The mixture was stirred under H₂ (15 psi) at 15° C. for 20 hours. The mixture was filtered and concentrated to give a residue. The residue was purified by column chromatography [SiO₂, Petroleum ether/Ethyl acetate 30:1 to 4:1] to afford the title compound (2.40 g, 55% yield) as a yellow oil: LCMS (ESI, M+1): m/z=297.2.

Step J. 5-ethyl-1-(tetrahydro-2H-pyran-2-yl)-1H-benzo[f]indazol-4-yl trifluoromethanesulfonate: To a solution of 5-ethyl-1-(tetrahydro-2H-pyran-2-yl)-1H-benzo[f]indazol-4-ol (2.40 g, 1.0 equiv) and DIPEA (3.14 g, 3.0 equiv) in DCM (30 mL) was added Tf₂O (4.57 g, 2.0 equiv) at −40° C. The mixture was stirred at −40° C. for 15 minutes. The mixture was concentrated and purified by column chromatography [SiO₂, Petroleum ether/Ethyl acetate 100:1 to 20:1] to afford the title compound (1.00 g, 26% yield) as a yellow solid; LCMS (ESI, M+1): m/z=429.1.

Step K. 5-ethyl-1-(tetrahydro-2H-pyran-2-yl)-4-(4,4,5,5-tetramethyl-1,3,2-dioxaborolan-2-yl)-1H-benzo[f]indazole: To the mixture of 5-ethyl-1-(tetrahydro-2H-pyran-2-yl)-1H-benzo[f]indazol-4-yl trifluoromethanesulfonate (1.00 g, 1.0 equiv), Pd(dppf)Cl₂ (171 mg, 0.1 equiv) and TEA (945 mg, 4.0 equiv) in ACN (20 mL) was added 4,4,5,5-tetramethyl-1,3,2-dioxaborolane (1.19 g, 4.0 equiv) under N₂. The mixture was stirred at 80° C. for 2 hours. The mixture was quenched by MeOH (3 mL) and concentrated to give a residue. The residue was purified by column chromatography [SiO₂, Petroleum ether/Ethyl acetate 80:1 to 10:1] to afford the title compound (190 mg, 18% yield) as a yellow oil; LCMS (ESI, M+1): m/z=407.2.

Step L. 5-ethyl-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole: To a mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (142 mg, 1.2 equiv), 5-ethyl-1-(tetrahydro-2H-pyran-2-yl)-4-(4,4,5,5-tetramethyl-1,3,2-dioxaborolan-2-yl)-1H-benzo[f]indazole (110 mg, 1.0 equiv) and Cs₂CO₃ (1.5 M, 3.0 equiv) in methoxycyclopentane (2 mL) was added CataCXium A Pd G3 (19.7 mg, 0.1 equiv) under N₂. The mixture was stirred at 90° C. for 2 hours. The mixture was diluted with water (5 mL) and extracted with ethyl acetate (2× 10 mL). The organic layer was dried over Na₂SO₄ and concentrated to give a residue. The residue was purified by reversed phase flash chromatography [C18, 0.1% formic acid condition] to afford the title compound (60.0 mg, 28% yield) as a yellow solid; LCMS (ESI, M+1): m/z=683.4.

### Intermediate 23

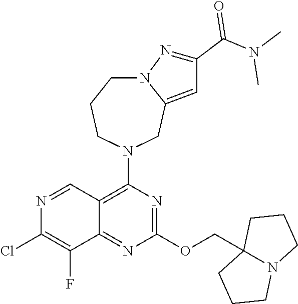 [SMILES: CN(C)C(=O)c1cc2n(n1)CCCN(c1nc(OCC34CCCN3CCC4)nc3c(F)c(Cl)ncc13)C2]

### 5-(7-chloro-8-fluoro-2-((hexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

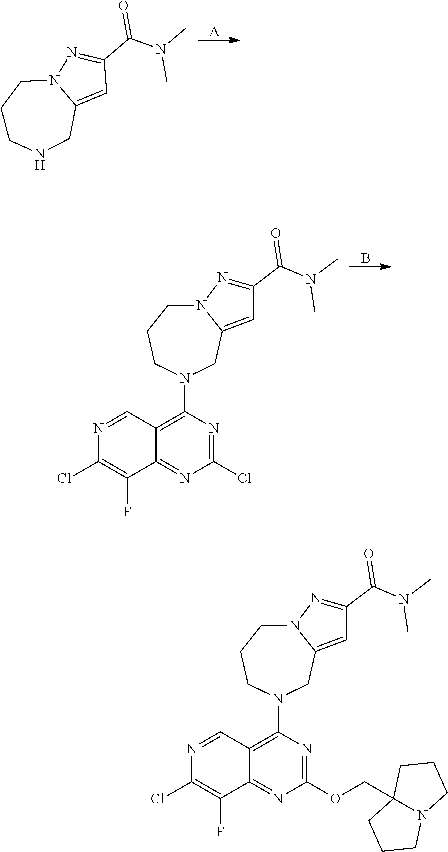

Step A. 5-(2,7-dichloro-8-fluoropyrido[4,3-d]pyrimidin-4-yl)-N,N-dimethyl-5,6,7,8-tetrahydro-4H-pyrazolo[1,5-a][1,4]diazepine-2-carboxamide: To a solution of 2,4,7-trichloro-8-fluoropyrido[4,3-d]pyrimidine (0.3 g, 1.0 equiv) and DIPEA (307 mg, 2.0 equiv) in DCM (6 mL) was added N,N-dimethyl-5,6,7,8-tetrahydro-4H-pyrazolo[1,5-a][1,4]diazepine-2-carboxamide (223 mg, 0.9 equiv) and DIPEA (614 mg, 4.0 equiv) in DMF (2 mL) dropwise at −40° C. The mixture was stirred at −40° C. for 0.5 hour. The mixture was quenched with H₂O (30 mL) at −40° C. The mixture was extracted with DCM (3×10 mL). The combined organic layer was dried over anhydrous Na₂SO₄ and concentrated. The residue was dispersed in petroleum ether/ethyl acetate 2:1 (30 mL). The mixture was stirred for 0.5 hour. The mixture was filtered and the solid was dried under reduced pressure to afford the title compound (0.48 g, 94% yield) as a yellow solid; LCMS (ESI, M+1, M+3, M+5): m/z=424.1, 426.1, 428.1.

Step B. 5-(7-chloro-8-fluoro-2-((hexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a mixture of 5-(2,7-dichloro-8-fluoropyrido[4,3-d]pyrimidin-4-yl)-N,N-dimethyl-5,6,7,8-tetrahydro-4H-pyrazolo[1,5-a][1,4]diazepine-2-carboxamide (240 mg, 1.0 equiv) and (hexahydro-1H-pyrrolizin-7a-yl)methanol (88 mg, 1.1 equiv) in dioxane (1 mL) and DMSO (1 mL) was added DIPEA (183 mg, 2.5 equiv) and 4A MS (30 mg). The mixture was stirred at 90° C. for 14 hours under N₂ atmosphere. The mixture was filtered and purified with reversed phase flash chromatography [water (FA, 0.1%)/acetonitrile 4:1] to afford the title compound (60 mg, 19% yield) as light yellow solid; LCMS (ESI, M+1, M+3): m/z=529.3, 531.3.

### Intermediate 24

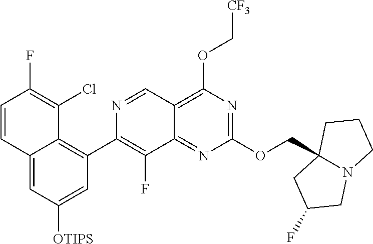

### 5-chloro-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol

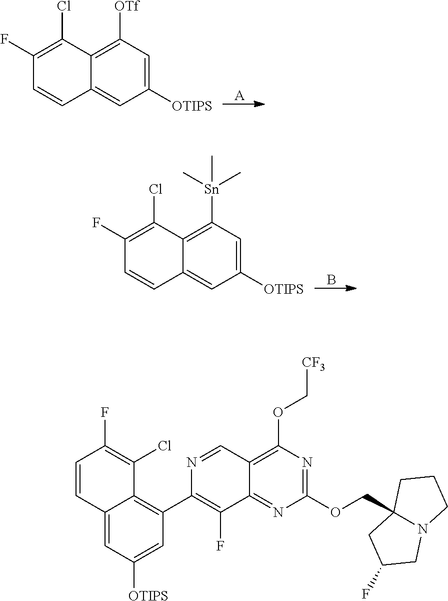

Step A. (5-chloro-6-fluoro-4-trimethylstannyl-2-naphthyl)oxy-triisopropyl-silane: A mixture of (8-chloro-7-fluoro-3-triisopropylsilyloxy-1-naphthyl)trifluoromethanesulfonate (800 mg, 1.0 equiv), trimethyl(trimethylstannyl) stannane (8.0 g, 15 equiv), Pd(PPh₃)₄ (184 mg, 0.1 equiv) and LiCl (203 mg, 3.0 equiv) in toluene (10 mL) was degassed and purged with N₂ for 3 times. And the mixture was stirred at 105° C. for 12 hours under N₂ atmosphere. The reaction was filtered and concentrated, purified by prep-HPLC [C18, 0.1% formic acid condition] and lyophilized to afford the title compound (150 mg, 18% yield) as a colorless liquid; ¹H NMR (400 MHz, CHLOROFORM-d) δ=7.62-7.57 (m, 1H), 7.52 (d, J=2.4 Hz, 1H), 7.30-7.25 (m, 2H), 7.19 (d, J=2.4 Hz, 1H), 1.37-1.27 (m, 5H), 1.15 (d, J=7.2 Hz, 18H), 0.50-0.36 (m, 9H).

Step B. 5-chloro-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol: A mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (300 mg, 1.0 equiv), (5-chloro-6-fluoro-4-trimethylstannyl-2-naphthyl)oxy-triisopropyl-silane (423 mg, 1.2 equiv), CuI (39.1 mg, 0.3 equiv), Pd(dppf)Cl₂ (50.0 mg, 0.1 equiv) and BINAP (85.2 mg, 0.2 equiv) in toluene (4.0 mL) was degassed and purged with N₂ for 3 times, and then the mixture was stirred at 20° C. for 12 hours under N₂ atmosphere. The reaction solution was filtered, and the filtrate was concentrated under reduced pressure to remove solvent. The crude product was purified by prep-HPLC [C18, 0.1% formic acid condition] to afford the title compound (80 mg, 15.5% yield) as a white solid. ¹H NMR (400 MHZ, CHLOROFORM-d) δ=9.25 (s, 1H), 7.70 (dd, J=5.4, 9.1 Hz, 1H), 7.37-7.30 (m, 2H), 7.24 (d, J=2.0 Hz, 1H), 5.41-5.19 (m, 1H), 5.17-4.95 (m, 2H), 4.45-4.25 (m, 2H), 4.13 (q, J=7.2 Hz, 1H), 3.31-3.18 (m, 2H), 3.04-2.96 (m, 1H), 2.32-2.22 (m, 1H), 2.18-2.11 (m, 1H), 2.02-1.93 (m, 2H), 1.83-1.51 (m, 3H), 1.36-1.30 (m, 3H), 1.13 (d, J=7.2 Hz, 17H), 0.91-0.78 (m, 2H).

### Intermediate 25

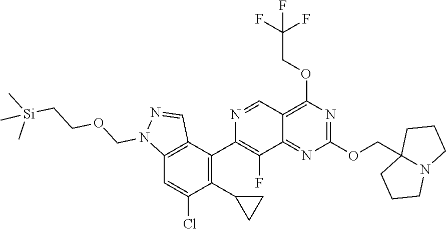 [SMILES: C[Si](C)(C)CCOCn1ncc2c(-c3ncc4c(OCC(F)(F)F)nc(OCC56CCCN5CCC6)nc4c3F)c(C3CC3)c(Cl)cc21]

### 7-(6-chloro-5-cyclopropyl-1-((2-(trimethylsilyl)ethoxy)methyl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine

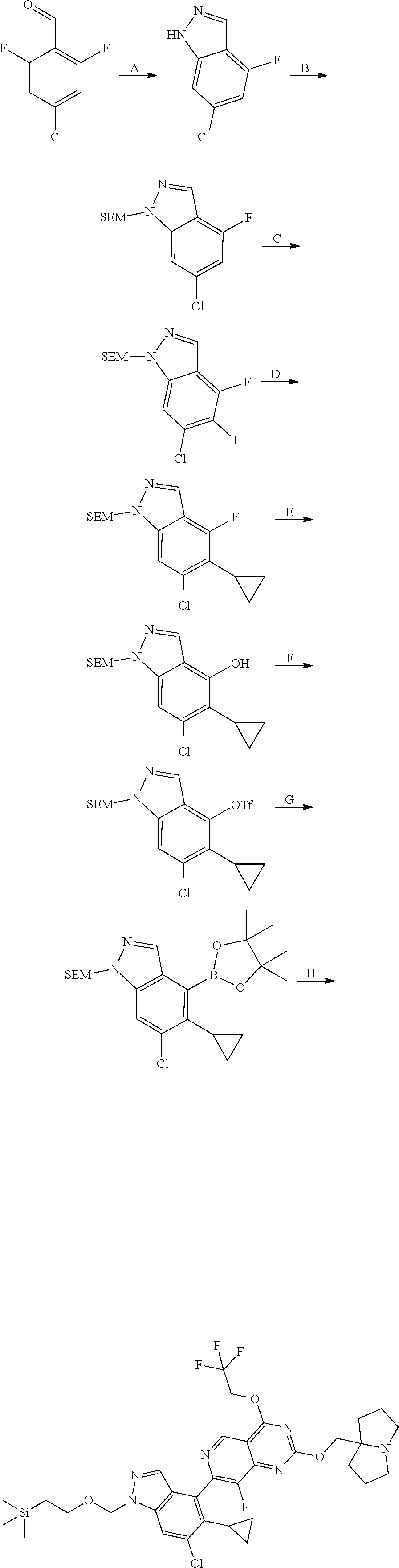

Step A. 6-chloro-4-fluoro-1H-indazole: To a solution of 4-chloro-2,6-difluoro-benzaldehyde (100 g, 1.0 equiv) in dioxane (1.0 L) was added N₂H₄·H₂O (58.1 g, 2.0 equiv) in dropwise at 25° C. for 10 minutes. The mixture was stirred at 25° C. for 0.5 hour, and 95° C. for 15.5 hours. The reaction mixture was diluted with H₂O (500 mL) and extracted with EtOAc (2×500 mL). The combined organic layers were washed with brine (200 mL), dried over anhydrous sodium sulfate, concentrated to afford the title compound (95.0 g, crude) as a white solid; ¹H NMR (400 MHZ, DMSO-d₆) δ=14.08-12.38 (m, 1H), 8.23 (d, J=0.4 Hz, 1H), 7.51 (s, 1H), 7.06 (dd, J=1.2, 9.6 Hz, 1H); LCMS (ESI, M+1): m/z=171.0.

Step B. 6-chloro-4-fluoro-1-((2-(trimethylsilyl)ethoxy)methyl)-1H-indazole: To a solution of 6-chloro-4-fluoro-1H-indazole (40.0 g, 1.0 equiv) in THF (200 mL) was added NaH (14.1 g, 60% purity 1.5 equiv) portionwise at 0° C. for 30 minutes. The mixture was stirred at 25° C. for 0.5 hour. Then SEM-Cl (46.9 g, 1.2 equiv) was added to the mixture in dropwise at 0° C. for 20 minutes. The mixture was stirred at 25° C. for 1 hour. The mixture was quenched by slow addition of H₂O (300 mL) at 0° C. over the course of 30 minutes. The mixture was extracted with EtOAc (2×300 mL). The combined organic layers were dried over anhydrous sodium sulfate, concentrated and purified by column chromatography [SiO₂, petroleum ether/ethyl acetate 1:0 to 10:1] to afford the title compound (42.0 g, 60% yield) as a yellow solid; ¹H NMR (400 MHZ, CDCl₃-d) δ=8.09-8.00 (m, 1H), 7.45-7.37 (m, 1H), 6.88 (dd, J=1.2, 9.6 Hz, 1H), 5.70 (s, 2H), 3.63-3.46 (m, 2H), 0.96-0.84 (m, 2H), −0.04 (s, 9H); LCMS (ESI, M+1): m/z=301.3.

Step C. 6-chloro-4-fluoro-5-iodo-1-((2-(trimethylsilyl)ethoxy)methyl)-1H-indazole: To a solution of 2-[(6-chloro-4-fluoro-indazol-1-yl)methoxy]ethyl-trimethyl-silane (20.0 g, 1.0 equiv) in THF (100 mL) was added LDA (43.2 mL, 1.3 equiv) in dropwise at −65° C. for 5 minutes. The mixture was stirred at −65° C. for 55 minutes. Then solution of 12 (21.9 g, 1.3 equiv) in THF (50.0 mL) was added to the mixture slowly at 15 minutes and stirred at −65° C. for 1 hour. The mixture was quenched with H₂O (100 mL) at 0° C. for 15 minutes. The mixture was extracted with EtOAc (2×100 mL). The combined organic layers were dried over anhydrous sodium sulfate, concentrated and purified by reversed phase flash chromatography [0.1% formic acid condition] to afford the title compound (12.0 g, 42% yield) as a yellow solid; ¹H NMR (400 MHz, CDCl₃-d) δ=8.03 (s, 1H), 7.61 (s, 1H), 5.70 (s, 2H), 3.58-3.50 (m, 2H), 0.93-0.86 (m, 2H), −0.04 (s, 9H). LCMS (ESI, M+1): m/z=427.2.

Step D. 6-chloro-5-cyclopropyl-4-fluoro-1-((2-(trimethylsilyl)ethoxy)methyl)-1H-indazole: To a solution of 2-[(6-chloro-4-fluoro-5-iodo-indazol-1-yl)methoxy]ethyl-trimethyl-silane (8.0 g, 1.0 equiv) and cyclopropylboronic acid (3.22 g, 2.0 equiv) in dioxane (80 mL) was added Pd(dppf)Cl₂ (1.37 g, 0.1 equiv) and K₃PO₄ (1.5 M, 37.5 mL, 3.0 equiv). The mixture was degassed and purged with N₂ three times and stirred at 100° C. for 12 hours under N₂. The mixture was diluted with H₂O (80 mL) and extracted with EtOAc (2×60 mL). The combined organic layers were dried over anhydrous sodium sulfate, concentrated and purified by column chromatography [SiO₂, petroleum ether/ethyl acetate 1:0 to 10:1] to afford the title compound (4.10 g, 58% yield) as a yellow oil. 1H NMR (400 MHZ, CDCl₃-d) δ=8.00 (d, J=0.8 Hz, 1H), 7.44 (s, 1H), 5.79-5.55 (m, 2H), 3.62-3.48 (m, 2H), 1.90-1.75 (m, 1H), 1.16-1.02 (m, 2H), 0.96-0.80 (m, 4H), 0.01 (s, 9H); LCMS (ESI, M+1): m/z=341.1.

Step E. 6-chloro-5-cyclopropyl-1-((2-(trimethylsilyl)ethoxy)methyl)-1H-indazol-4-ol: To a solution of 2-[(6-chloro-5-cyclopropyl-4-fluoro-indazol-2-yl)methoxy]ethyl-trimethyl-silane (4.10 g, 1.0 equiv) and 2-methylsulfonylethanol (2.20 g, 1.5 equiv) in DMF (50 mL) was added NaH (2.40 g, 60% purity, 5.0 equiv) portionwise at 0° C. for 15 minutes. Then the mixture was stirred at 25° C. for 0.5 hour and 40° C. for 11.5 hours. The mixture was quenched by slow addition of H₂O (50 mL) at 0° C. over the course of 15 minutes. The mixture was extracted with EtOAc (2×10 mL). The combined organic layers were dried over anhydrous sodium sulfate, concentrated and purified by reversed phase flash chromatography [0.1% formic acid condition] to afford the title compound (2.0 g, 50% yield) as a yellow oil. 1H NMR (400 MHZ, CDCl₃-d) δ=8.12-7.98 (m, 1H), 7.20 (s, 1H), 6.54 (s, 1H), 5.80-5.58 (m, 2H), 3.66-3.41 (m, 2H), 1.78-1.60 (m, 1H), 1.27-1.14 (m, 2H), 0.98-0.83 (m, 2H), 0.80-0.69 (m, 2H), −0.04 (s, 9H); LCMS (ESI, M+1): m/z=339.3.

Step F. 6-chloro-5-cyclopropyl-1-((2-(trimethylsilyl)ethoxy)methyl)-1H-indazol-4-yl trifluoromethanesulfonate: To a solution of 6-chloro-5-cyclopropyl-1-(2-trimethylsilylethoxymethyl) indazol-4-ol (0.80 g, 1.0 equiv) 4 Å molecular sieves (100 mg), DIPEA (915 mg, 3.0 equiv) in DCM (8 mL) was added Tf₂O (999 mg, 1.5 equiv). The mixture was degassed and purged with N₂ three times, and stirred at −40° C. for 0.5 hour. The mixture was diluted with H₂O (3 mL) and extracted with EtOAc (2×5 mL). The combined organic layers were dried over anhydrous sodium sulfate, concentrated and purified by column chromatography [SiO₂, petroleum ether/ethyl acetate 1:0 to 10:1] to afford the title compound (740 mg, 62% yield) as a yellow oil. ¹H NMR (400 MHZ, CDCl₃-d) δ=7.98 (s, 1H), 7.70 (s, 1H), 5.70 (s, 2H), 3.55 (t, J=8.4 Hz, 2H), 1.94-1.82 (m, 1H), 1.31-1.19 (m, 2H), 0.90 (t, J=8.4 Hz, 2H), 0.81-0.70 (m, 2H), −0.05 (s, 9H); LCMS (ESI, M+1): m/z=471.1.

Step G. 6-chloro-5-cyclopropyl-4-(4,4,5,5-tetramethyl-1,3,2-dioxaborolan-2-yl)-1-((2-(trimethylsilyl)ethoxy)methyl)-1H-indazole: To a solution of 6-chloro-5-cyclopropyl-1-((2-(trimethylsilyl)ethoxy)methyl)-1H-indazol-4-yl trifluoromethanesulfonate (300 mg, 1.0 equiv) and Pin₂B₂ (243 mg, 1.5 equiv) in ACN (6 mL) were added KOAc (125 mg, 2.0 equiv), P(Cy₃)-Pd-G3 (46.8 mg, 0.1 equiv) under N₂. The mixture was stirred at 90° C. for 12 hours. The mixture was filtered, concentrated and purified by reversed phase flash chromatography [C18, 0.1% formic acid condition] to afford the title compound (150 mg, 51% yield) as a yellow liquid; ¹H NMR (400 MHZ, METHANOL-d₄) δ=8.13 (d, J=0.8 Hz, 1H), 7.65 (d, J=0.4 Hz, 1H), 5.67 (s, 2H), 3.55-3.50 (m, 2H), 2.19-2.10 (m, 1H), 1.47 (s, 12H), 1.10-1.04 (m, 2H), 0.9-0.86 (m, 2H), 0.62-0.56 (m, 2H), −0.05 (s, 9H); LCMS (ESI, M+1): m/z=449.2.

Step H. 7-(6-chloro-5-cyclopropyl-1-((2-(trimethylsilyl)ethoxy)methyl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine: To a solution of 7-chloro-8-fluoro-2-((hexahydro-1H-pyrrolizin-7a-yl)methoxy)-4-(2,2,2-trifluoroethoxy)pyrido[4,3-d]pyrimidine (150 mg, 1.0 equiv) and 6-chloro-5-cyclopropyl-4-(4,4,5,5-tetramethyl-1,3,2-dioxaborolan-2-yl)-1-((2-(trimethylsilyl)ethoxy)methyl)-1H-indazole (192 mg, 1.2 equiv) in toluene (3 mL) were added K₃PO₄ (1.5 M, 713 μL, 3.0 equiv) and APhos-Pd-G3 (22.6 mg, 0.1 equiv). The mixture was stirred at 60° C. for 12 hours under N₂. The reaction mixture was diluted with H₂O (10 mL) and extracted with EtOAc (3×10 mL). The combined organic layers were dried over anhydrous sodium sulfate, concentrated and purified by column chromatography [SiO₂, Petroleum ether/Ethyl acetate 10:1 to 1:0] and reversed phase flash chromatography [C18, 0.1% formic acid condition] to afford the title compound (70.0 mg, 28% yield) as yellow liquid.

### Intermediate 26

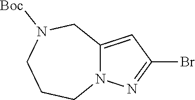 [SMILES: CC(C)(C)OC(=O)N1CCCn2nc(Br)cc2C1]

### tert-butyl 2-bromo-7,8-dihydro-4H-pyrazolo[1,5-a][1,4]diazepine-5(6H)-carboxylate

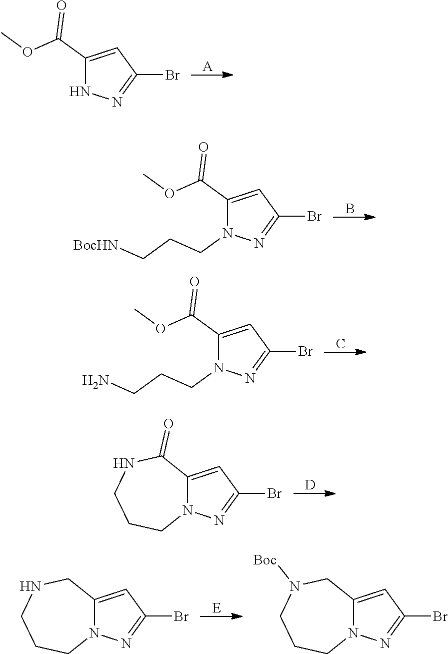

Step A. methyl 3-bromo-1-(3-((tert-butoxycarbonyl)amino) propyl)-1H-pyrazole-5-carboxylate: To a solution of methyl 3-bromo-1H-pyrazole-5-carboxylate (1.0 g, 1.0 equiv) in ACN (10.0 mL) were added Cs₂CO₃ (3.2 g, 2.0 equiv) and tert-butyl N-(3-bromopropyl)carbamate (1.4 g, 1.2 equiv). The reaction was stirred at 15° C. for 2 hours. The mixture was filtered and purified by flash silica gel chromatography (ISCO®; 12 g SepaFlash® Silica Flash Column, Eluent of 0˜30% Ethyl acetate/Petroleum ethergradient @36 mL/min) to afford the title compound (1.3 g, 68.3% yield, 95% purity) as a colorless clear liquid. 1H NMR (400 MHz, CHLOROFORM-d) δ=6.81 (s, 1H), 4.88 (br s, 1H), 4.67-4.57 (m, 2H), 3.96-3.85 (m, 3H), 3.10 (br d, J=5.6 Hz, 2H), 2.09-1.98 (m, 3H), 1.45 (s, 10H)

Step B. methyl 1-(3-aminopropyl)-3-bromo-1H-pyrazole-5-carboxylate: To a solution methyl 3-bromo-1-(3-((tert-butoxycarbonyl)amino) propyl)-1H-pyrazole-5-carboxylate (1.3 g, 1.0 equiv) in dioxane (5.0 mL) was added HCl·dioxane (4 M, 15 mL, 17.1 equiv). The reaction was stirred at 20° C. for 12 hours. The mixture was concentrated under reduced pressure to afford the title compound (1.1 g, HCl) as a white solid.

Step C. 2-bromo-5,6,7,8-tetrahydro-4H-pyrazolo[1,5-a][1,4]diazepin-4-one; A solution of methyl 1-(3-aminopropyl)-3-bromo-1H-pyrazole-5-carboxylate hydrochloride (1.1 g, 1.0 equiv) in saturated Na₂CO₃ (20 mL) solution was stirred at 20° C. for 12 hours. The reaction was diluted with H₂O (30 mL) and extracted with DCM 100 mL (20 mL×5). The combined organic layers were washed with brine (30 mL), dried over Na₂SO₄, filtered, concentrated, and triturated with DCM/PE at 20° C. for 5 minutes to afford the title compound (690 mg. 79.3% yield). ¹H NMR (400 MHZ, CHLOROFORM-d) δ=7.00-6.93 (m, 1H), 6.84 (s, 1H), 4.49 (t, J=6.8 Hz, 2H), 3.43-3.38 (m, 3H), 2.32-2.22 (m, 3H); LCMS (ESI, M+1): m/z=232.0.

Step D. 2-bromo-5,6,7,8-tetrahydro-4H-pyrazolo[1,5-a][1,4]diazepine: A mixture of 2-bromo-5,6,7,8-tetrahydro-4H-pyrazolo[1,5-a][1,4]diazepin-4-one (610 mg, 1.0 equiv) in THF (5.0 mL) was degassed and purged with N₂ 3 times. BH₃·Me₂S (10 M, 3.0 equiv) was added at 0° C. and the reaction was stirred at 20° C. for 30 minutes then at 60° C. for 12 hours. The mixture was concentrated under reduced pressure to afford the title compound (650 mg, 97% yield, HCl) as a white solid. ¹H NMR (400 MHZ, DMSO-d6) δ=6.55 (s, 1H), 4.48-4.34 (m, 5H), 3.38 (br dd, J=6.0, 12.0 Hz, 3H), 2.01 (br d, J=4.0 Hz, 2H).

Step E. tert-butyl 2-bromo-7,8-dihydro-4H-pyrazolo[1,5-a][1,4]diazepine-5(6H)-carboxylate: To a solution of 2-bromo-5,6,7,8-tetrahydro-4H-pyrazolo[1,5-a][1,4]diazepine (650 mg, 1.0 equiv, HCl) in DCM (8 mL) were added TEA (520 mg, 716 μL 2.0 equiv) and Boc₂O (842 mg, 1.5 equiv). The mixture was stirred at 25° C. for 2 hours. The reaction was concentrated and purified by flash silica gel chromatography (ISCO®; 12 g SepaFlash® Silica Flash Column, Eluent of 0˜30% Ethyl acetate/Petroleum ethergradient @ 30 mL/min) to afford the title compound (250 mg, 28.9% yield, 94.0% purity) as a white solid. 1H NMR (400 MHZ, CHLOROFORM-d) δ=6.32-6.12 (m, 1H), 4.48-4.35 (m, 4H), 3.78-3.65 (m, 2H), 1.91 (br d, J=4.4 Hz, 2H), 1.51-1.40 (m, 9H); LCMS (ESI, M+1): m/z=318.0.

### Intermediate 27

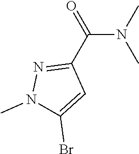

### 5-bromo-N,N,1-trimethyl-1H-pyrazole-3-carboxamide

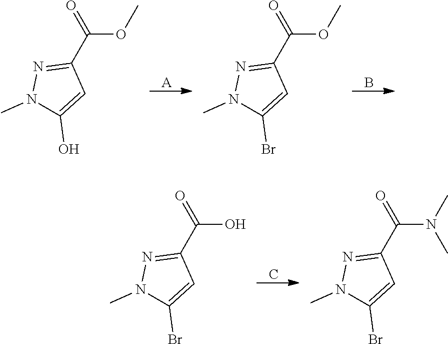

Step A. methyl 5-bromo-1-methyl-1H-pyrazole-3-carboxylate: To a solution of methyl 5-hydroxy-1-methyl-1H-pyrazole-3-carboxylate (25.0 g, 1.0 equiv) in acetonitrile (250 mL) was added POBr₃ (184 g, 4.0 equiv) in portions under N₂ at 0° C. The reaction was stirred at 80° C. for 12 hours. The mixture was diluted with ethyl acetate (500 mL) and washed with 5% NaHCO₃ solution (3×300 mL) and brine (200 mL). The organic layer was dried over anhydrous sodium sulfate, concentrated, and purified with column chromatography (SiO₂, petroleum ether/ethyl acetate=1/0 to 4/1) to afford the title compound (13.0 g, 34% yield) as yellow solid.

Step B. 5-bromo-1-methyl-1H-pyrazole-3-carboxylic acid: To a solution of methyl 5-bromo-1-methyl-1H-pyrazole-3-carboxylate (13.0 g, 1.0 equiv) in THF (237 mL) was added NaOH (2 M in water, 119 mL, 4.0 equiv) in one portion. The reaction was stirred at 25° C. for 2 hours. The mixture was quenched with conc. HCl (150 mL) and extracted with ethyl acetate (3×50 mL). The combined organic layers were washed with brine (50 mL), dried over anhydrous sodium sulfate, concentrated to afford the title compound (5.00 g, crude) as white solid; LCMS (ESI, M+1, M+3): m/z=204.9, 206.9.

Step C. 5-bromo-N,N,1-trimethyl-1H-pyrazole-3-carboxamide: To a mixture of 5-bromo-1-methyl-1H-pyrazole-3-carboxylic acid (5.00 g, 1.0 equiv) in THF (50 mL) was added dimethylamine (2 M in THF, 24.4 mL, 2.0 equiv) and DIEA (15.8 g, 5.0 equiv) at 0° C. HATU (18.5 g, 2.0 equiv) was added. The reaction was stirred at 25° C. 0.5 hours. The mixture was diluted with water (100 mL) and extracted with ethyl acetate (3×50 mL). The combined organic layers were washed with brine (30 mL), dried over anhydrous sodium sulfate, concentrated, and purified by column chromatography (SiO₂, petroleum ether/ethyl acetate=I/O to 2/1) to afford the title compound (4.10 g, 72% yield) as yellow solid; LCMS (ESI, M+1, M+3): m/z=232.0, 234.0.

### Intermediate 28

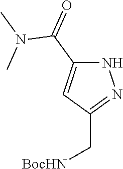 [SMILES: CN(C)C(=O)c1cc(CNC(=O)OC(C)(C)C)n[nH]1]

### tert-butyl ((5-(dimethylcarbamoyl)-1H-pyrazol-3-yl)methyl)carbamate

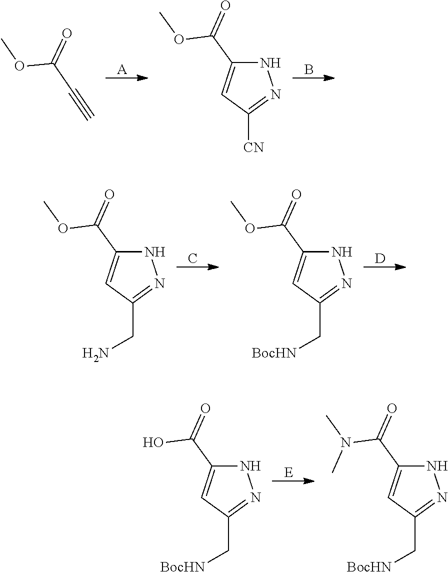

Step A. methyl 3-cyano-1H-pyrazole-5-carboxylate: To a solution of methyl prop-2-ynoate (5.00 g, 1.0 equiv) and 2-aminoacetonitrile (9.91 g, 1.8 equiv, HCl) in CHCl₃ (500 mL) and H₂O (16 mL) was added NaNO₂ (12.3 g, 3.0 equiv) at 25° C. slowly. The reaction was stirred at 25° C. for 3 hours and warmed to 60° C. for 12 hours. The reaction mixture was filtered, concentrated, and purified by flash silica gel chromatography [ISCO®; 80 g Sepaflash® silica flash column, eluent of 30˜50% EtOAc/PE gradient] to afford the title compound (5.00 g, 54% yield) as yellow solid; 1H NMR (400 MHZ, CHLOROFORM-d) δ=11.59 (br s, 1H), 7.23 (s, 1H), 4.01 (s, 3H).

Step B. methyl 3-(aminomethyl)-1H-pyrazole-5-carboxylate: To a solution of methyl 3-cyano-1H-pyrazole-5-carboxylate (500 mg, 1.0 equiv) in MeOH (10 mL) were added HCl (0.5 mL) and Pd/C (50.0 mg, 10% purity, wet). The reaction was stirred at 25° C. under H₂ atmosphere for 2 hours. The mixture was filtered and concentrated to afford the title compound (513 mg, 99% yield) as yellow oil.

Step C. methyl 3-[(tert-butoxycarbonylamino)methyl]-1H-pyrazole-5-carboxylate: To a solution of methyl 3-(aminomethyl)-1H-pyrazole-5-carboxylate (400 mg, 79.7% purity, 1.0 equiv) in DCM (10 mL) were added TEA (424 mg, 3.0 equiv) and Boc₂O (336 mg, 1.1 equiv). The reaction was stirred at 25° C. for 1 hour. The mixture was filtered, concentrated, and purified by flash silica gel chromatography [ISCOR; 20 g Sepaflash® silica flash column, eluent of 0˜65% PE/EtOAc gradient @20 mL/min] to afford the title compound (300 mg, 83% yield) as white solid; LCMS (ESI, M+1): m/z=256.2.

Step D. 3-[(tert-butoxycarbonylamino)methyl]-1H-pyrazole-5-carboxylic acid: To a solution of methyl 3-[(tert-butoxycarbonylamino)methyl]-1H-pyrazole-5-carboxylate (300 mg, 1.0 equiv) in MeOH (10 mL) was added NaOH (1 M, 3.5 mL, 3.0 equiv) in water. The reaction was stirred at 60° C. for 2 hours. The mixture was poured into water (20 mL) and the pH was adjusted to 5 with HCl (1 M). The mixture was extracted with EtOAc (3×20 mL). The combined organic layers were dried over anhydrous sodium sulfate and concentrated to afford the title compound (280 mg, 99% yield) as white solid; ¹H NMR (400 MHZ, DMSO-d₆) δ=13.32-12.96 (m, 1H), 7.35-7.26 (m, 1H), 6.53 (br s, 1H), 4.11 (br d, J=5.2 Hz, 2H), 1.39 (s, 9H).

Step E. tert-butyl N-[[5-(dimethylcarbamoyl)-1H-pyrazol-3-yl]methyl]carbamate: To a solution of 3-[(tert-butoxycarbonylamino)methyl]-1H-pyrazole-5-carboxylic acid (260 mg, 1.0 equiv) and N-methylmethanamine (439 mg, 5.0 equiv, HCl) in DMAc (10 mL) was added dropwise TEA (240 mg, 2.2 equiv). Then bromo(tripyrrolidin-1-yl)phosphonium;hexafluorophosphate (653 mg, 1.3 equiv) was added to the mixture. The reaction was stirred at 40° C. for 12 hours. The mixture was filtered and purified with prep-HPLC [Waters Xbridge 150×25 mm×5 um; A: water (NH₃·H₂O), B: ACN, B %: 10%-40% over 10 min] to afford the title compound (170 mg, 59% yield) as yellow gum; ¹H NMR (400 MHZ, METHANOL-d₄) δ=6.56-6.43 (m, 1H), 4.28 (br s, 2H), 3.27 (s, 3H), 3.09 (s, 3H), 1.45 (s, 9H).

### Intermediate 29

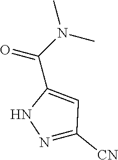

### 3-cyano-N,N-dimethyl-1H-pyrazole-5-carboxamide

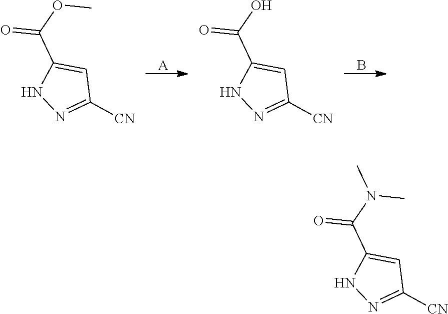

Step A. 3-cyano-1H-pyrazole-5-carboxylic acid: To a solution of methyl 3-cyano-1H-pyrazole-5-carboxylate (500 mg, 1.0 equiv) in water (15.0 mL) and tetrahydrofuran (15.0 mL) was added lithium hydroxide (158 mg, 2.0 equiv). The reaction was stirred at 50° C. for 2 hours. The mixture was quenched with water (20.0 mL) and extracted with ethyl acetate (3×20.0 mL). The combined organic layers were dried over anhydrous sodium sulfate and concentrated to give the title compound (400 mg, 88% yield) as yellow oil; ¹H NMR (400 MHZ, DMSO-d₆) δ ppm 7.27-7.51 (m, 1H).

Step B. 3-cyano-N,N-dimethyl-1H-pyrazole-5-carboxamide: To a solution of 3-cyano-N,N-dimethyl-1H-pyrazole-5-carboxamide (500 mg, 1.0 equiv) and N-methylmethanamine (2 M, 2.0 equiv, tetrahydrofuran) in N,N-dimethylformamide (25 mL) were added N,N-diisopropylethylamine (1.41 g, 3.0 equiv) and [dimethylamino (triazolo[4,5-b]pyridin-3-yloxy)methylidene]-dimethylazanium; hexafluorophosphate (2.08 g, 1.50 equiv). The reaction was stirred at 25° C. for 1 hour. The mixture was concentrated and purified with prep-HPLC [neutral condition; column: Waters Xbridge C18 150×50 mm×10 um; mobile phase: [water (NH₄HCO₃)-ACN]; B %: 1%-26%, 10 min] to afford the title compound (500 mg, 84% yield) as yellow solid; ¹HNMR (400 MHz, CHLOROFORM-d) δ=2.82 (s, 3H) 2.91 (s, 3H) 7.95 (s, 1H).

### Intermediate 30

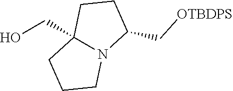

### ((3R,7aS)-3-(((tert-butyldiphenylsilyl)oxy)methyl)hexahydro-1H-pyrrolizin-7a-yl)methanol

### Intermediate 31

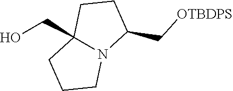

### ((3S,7aR)-3-(((tert-butyldiphenylsilyl)oxy)methyl)hexahydro-1H-pyrrolizin-7a-yl)methanol

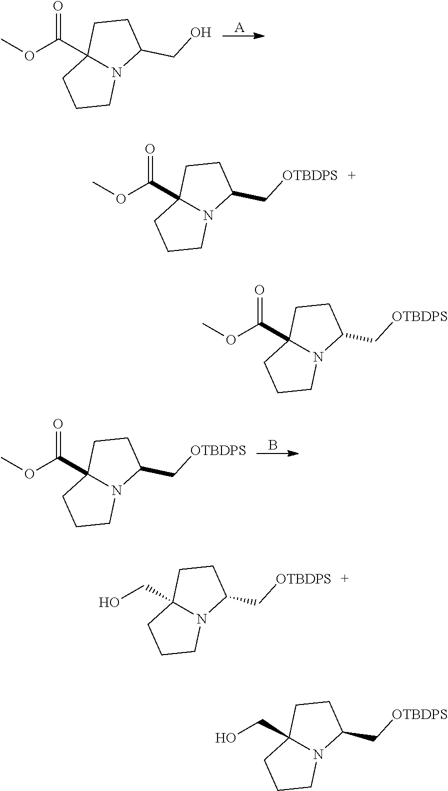

Step A. methyl cis-3-(((tert-butyldiphenylsilyl)oxy)methyl)tetrahydro-1H-pyrrolizine-7a(5H)-carboxylate methyl and trans-3-(((tert-butyldiphenylsilyl)oxy)methyl)tetrahydro-1H-pyrrolizine-7a(5H)-carboxylate: To a solution of methyl 3-(hydroxymethyl)tetrahydro-1H-pyrrolizine-7a(5H)-carboxylate (10 g, 1 equiv) and imidazole (10.2 g, 3 equiv) in DCM (150 mL) were added TBDPSCl (20.7 g, 1.5 equiv) and DMAP (613 mg, 0.1 equiv). The reaction was stirred at 25° C. for 2 hours. The mixture was washed with H₂O (2×50 mL). The organic layer was dried over anhydrous sodium sulfate, filtered, concentrated, and purified with column chromatography (SiO₂, Petroleum ether: Ethyl acetate=50:1-1:1) and reversed phase flash chromatography (C18, 0.1% formic acid condition) to afford the two title compounds: Peak 1 (3.4 g, 12% yield) as yellow oil; LCMS (ESI, M+1): m/z=438.3. Peak 2 (4 g, 13% yield) as yellow oil; LCMS (ESI, M+1): m/z=438.3.

Step B. ((3R,7aS)-3-(((tert-butyldiphenylsilyl)oxy)methyl)tetrahydro-1H-pyrrolizin-7a(5H)-yl)methanol and ((3S,7aR)-3-(((tert-butyldiphenylsilyl)oxy)methyl)tetrahydro-1H-pyrrolizin-7a(5H)-yl)methanol: To a solution of methyl cis-3-(((tert-butyldiphenylsilyl)oxy)methyl)tetrahydro-1H-pyrrolizine-7a(5H)-carboxylate (5.0 g, 1.0 equiv) in THF (60 mL) was added LiAlH₄ (1.30 g, 3.0 equiv) in portions at −40° C. The reaction was stirred at −40° C. for 1 hour. The mixture was quenched with saturated anhydrous sodium sulfate aqueous solution (4 mL), filtered, concentrated and purified with reversed phase flash chromatography (C18, 0.1% formic acid condition) followed with SFC [column: Phenomenex-Cellulose-2 (250 mm×30 mm, 10 um); mobile phase: (0.1% NH₃H₂O IPA); B %: 50%-50%, 7.7 min; 470 min] to afford the two title compounds:

Intermediate 30 (900 mg, 19% yield) as yellow oil; ¹H NMR (400 MHZ, chloroform-d) δ=7.70 (br t, J=5.6 Hz, 4H), 7.50-7.32 (m, 6H), 3.73-3.52 (m, 2H), 3.33-3.11 (m, 2H), 2.98-2.71 (m, 3H), 1.98-1.84 (m, 2H), 1.83-1.73 (m, 2H), 1.71-1.64 (m, 2H), 1.63-1.54 (m, 2H), 1.07 (s, 9H).

Intermediate 31 (900 mg, 19% yield) as yellow oil; ¹H NMR (400 MHZ, chloroform-d) δ=7.74-7.67 (m, 4H), 7.46-7.37 (m, 6H), 3.74-3.52 (m, 2H), 3.37-3.11 (m, 2H), 3.03-2.71 (m, 3H), 2.01-1.88 (m, 2H), 1.87-1.78 (m, 2H), 1.74-1.65 (m, 2H), 1.63-1.52 (m, 2H), 1.07 (s, 9H).

### Intermediate 32

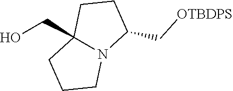

### ((3R,7aR)-3-(((tert-butyldiphenylsilyl)oxy)methyl)hexahydro-1H-pyrrolizin-7a-yl)methanol

### Intermediate 33

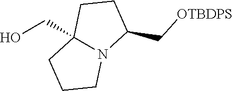

### ((3S,7aS)-3-(((tert-butyldiphenylsilyl)oxy)methyl)hexahydro-1H-pyrrolizin-7a-yl)methanol

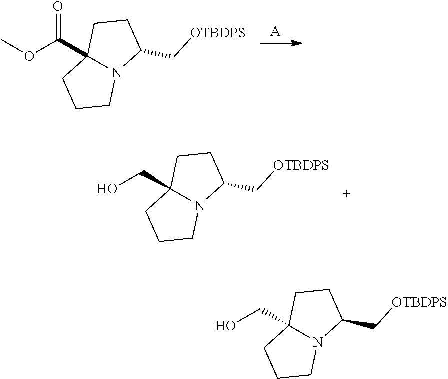

Step A. ((3R,7aR)-3-(((tert-butyldiphenylsilyl)oxy)methyl)tetrahydro-1H-pyrrolizin-7a(5H)-yl)methanol and ((3S,7aS)-3-(((tert-butyldiphenylsilyl)oxy)methyl)tetrahydro-1H-pyrrolizin-7a(5H)-yl)methanol: To a solution of methyl trans-3-(((tert-butyldiphenylsilyl)oxy)methyl)tetrahydro-1H-pyrrolizine-7a(5H)-carboxylate (3.0 g, 1.0 equiv) in THF (40 mL) was added LiAlH₄ (781 mg, 3.0 equiv) at −40° C. in portions. The reaction was stirred at −40° C. for 1 hour. The mixture was quenched with saturated anhydrous sodium sulfate aqueous solution (3 mL), filtered, concentrated and purified with reversed phase flash chromatography (C18, 0.1% formic acid condition) followed by SFC [column: DAICEL CHIRALPAK IG (250 mm×30 mm, 10 um); mobile phase: (0.1% NH₃H₂O MeOH); B %: 30%-30%, 3; 800 min] to afford the two title compounds:

Intermediate 32 (1.0 g, 36% yield) as yellow oil; 1H NMR (400 MHZ, chloroform-d) δ=7.68 (td, J=1.6, 8.0 Hz, 4H), 7.49-7.35 (m, 6H), 3.84-3.65 (m, 2H), 3.33 (br s, 2H), 3.26-3.15 (m, 1H), 2.93-2.89 (m, 1H), 2.77 (br d, J=6.4 Hz, 1H), 2.03-1.95 (m, 1H), 1.84-1.72 (m, 4H), 1.71-1.59 (m, 3H), 1.07 (s, 9H).

Intermediate 33 (1.0 g, 36% yield) as yellow oil; ¹H NMR (400 MHZ, chloroform-d) δ=7.67 (br d, J=7.6 Hz, 4H), 7.48-7.37 (m, 6H), 3.95-3.88 (m, 1H), 3.80 (br dd, J=5.6, 10.8 Hz, 1H), 3.34 (br s, 2H), 3.29-3.19 (m, 1H), 3.00-2.88 (m, 1H), 2.85-2.72 (m, 1H), 2.04-1.95 (m, 1H), 1.84-1.72 (m, 4H), 1.71-1.57 (m, 3H), 1.07 (s, 9H).

### Intermediate 34

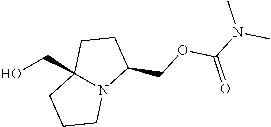 [SMILES: CN(C)C(=O)OC[C@@H]1CC[C@@]2(CO)CCCN12]

### ((3S,7aR)-7a-(hydroxymethyl)hexahydro-1H-pyrrolizin-3-yl)methyl dimethylcarbamate

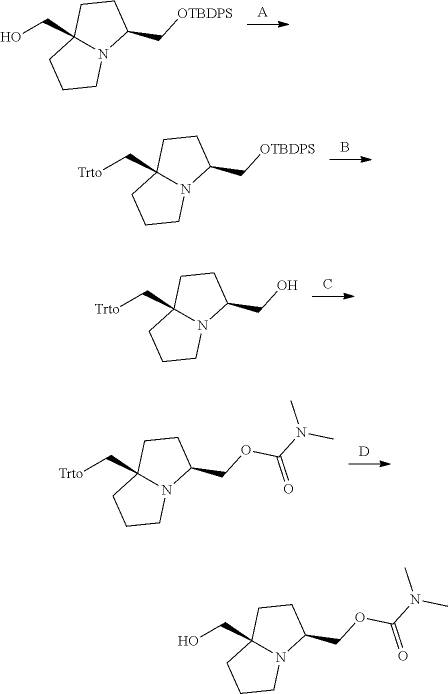

Step A. (3S,7aR)-3-(((tert-butyldiphenylsilyl)oxy)methyl)-7a-((trityloxy)methyl)hexahydro-1H-pyrrolizine: To a solution of ((3S,7aR)-3-(((tert-butyldiphenylsilyl)oxy)methyl)tetrahydro-1H-pyrrolizin-7a(5H)-yl)methanol (6.44 g, 1.0 equiv) and TEA (3.98 g, 2.5 equiv) in DCM (64.4 mL) was added TrtCl (8.77 g, 2.0 equiv) at 0° C. The reaction was stirred at 15° C. for 12 hours. The mixture was diluted with water (100 mL) and extracted with ethyl acetate (3×50 mL). The combined organic layers were washed with brine (2× 40 mL), dried over Na₂SO₄, filtered, and concentrated to afford the title compound (10.3 g, crude) as a yellow oil; LCMS (ESI, M+1): m/z=652.8.

Step B. ((3S,7aR)-7a-((trityloxy)methyl)hexahydro-1H-pyrrolizin-3-yl)methanol: To a solution of (3S,7aR)-3-(((tert-butyldiphenylsilyl)oxy)methyl)-7a-((trityloxy)methyl)hexahydro-1H-pyrrolizine (10.3 g, 1.0 equiv) in DMF (20.3 mL) was added CsF (23.9 g, 10 equiv). The reaction was stirred at 25° C. for 12 hours. The mixture was diluted with water (60 mL) and extracted with ethyl acetate (3×20 mL). The combined organic layers were washed with brine (2×20 mL), dried over anhydrous sodium sulfate, filtered, concentrated, and purified with reversed-phase flash chromatography (0.1% FA condition) to afford the title compound (4.15 g, 57% yield) as a yellow oil; ¹H NMR (400 MHz, CDCl₃) δ=7.49-7.38 (m, 6H), 7.29-7.17 (m, 9H), 3.44 (dd, J=4.4, 10.4 Hz, 1H), 3.27 (br dd, J=3.6, 10.8 Hz, 1H), 2.95-2.83 (m, 3H), 2.82-2.73 (m, 1H), 2.62 (td, J=6.0, 11.2 Hz, 1H), 2.02 (s, 1H), 1.89-1.81 (m, 1H), 1.78-1.48 (m, 6H).

Step C. ((3S,7aR)-7a-((trityloxy)methyl)hexahydro-1H-pyrrolizin-3-yl)methyl dimethylcarbamate: To a solution of ((3S,7aR)-7a-((trityloxy)methyl)hexahydro-1H-pyrrolizin-3-yl)methanol (4.15 g, 1.0 equiv) and TEA (3.05 g, 3.0 equiv) in DCM (42 mL) was added (4-nitrophenyl) carbonochloridate (3.03 g, 1.5 equiv) at 0-5° C. under N₂. The reaction was stirred at 20° C. for 2 hours. N-methylmethanamine (2.0 M, 7.5 mL, 1.5 equiv) was added to the mixture at 0° C. under N₂. The reaction was stirred at 0° C. for 0.5 hour. The mixture was diluted with water (50 mL) and extracted with DCM (3×50 mL). The combined organic layers were dried over anhydrous sodium sulfate, filtered, concentrated, and purified by reversed-phase flash chromatography (0.1% FA condition) to afford the title compound (2.03 g, 41% yield) as a yellow oil; LCMS (ESI, M+1): m/z=485.7.

Step D. ((3S,7aR)-7a-(hydroxymethyl)hexahydro-1H-pyrrolizin-3-yl)methyl dimethylcarbamate: To a solution of ((3S,7aR)-7a-((trityloxy)methyl)hexahydro-1H-pyrrolizin-3-yl)methyl dimethylcarbamate (2.03 g, 1.0 equiv) in DCM (20 mL) was added TFA (4.78 g, 10 equiv). The reaction was stirred at 0-25° C. for 12 hours. The mixture was concentrated, dissolved in MeOH (10 mL), neutralized with solid NaHCO₃ and purified by column chromatography [Al₂O₃, petroleum ether/ethyl acetate=10/1 to 1/1, dichloromethane/methanol=15:1] to afford the title compound (834 mg, 82% yield) as a yellow oil; ¹H NMR (400 MHZ, METHANOL-d4) δ=4.03-3.93 (m, 2H), 3.41-3.18 (m, 3H), 3.04-2.97 (m, 1H), 2.97-2.84 (m, 6H), 2.80 (td, J=4.8, 10.4 Hz, 1H), 2.09-1.93 (m, 2H), 1.91-1.82 (m, 1H), 1.82-1.71 (m, 2H), 1.69-1.47 (m, 3H).

### Intermediate 35

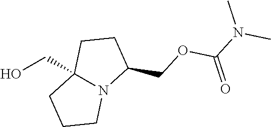 [SMILES: CN(C)C(=O)OC[C@@H]1CC[C@]2(CO)CCCN12]

### ((3S,7aS)-7a-(hydroxymethyl)hexahydro-1H-pyrrolizin-3-yl)methyl dimethylcarbamate

Synthesized according to Intermediate 34 from ((3S,7aS)-3-(((tert-butyldiphenylsilyl)oxy)methyl)hexahydro-1H-pyrrolizin-7a-yl)methanol. The title compound was obtained as yellow oil; ¹H NMR (400 MHZ, METHANOL-d₄) δ=4.00 (dq, J=6.0, 10.8 Hz, 2H), 3.31-3.22 (m, 2H), 3.03-2.96 (m, 2H), 2.95-2.86 (m, 6H), 2.80 (td, J=5.2, 10.8 Hz, 1H), 2.09-1.46 (m, 8H).

### Intermediate 36

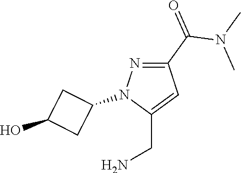 [SMILES: CN(C)C(=O)c1cc(CN)n([C@H]2C[C@H](O)C2)n1]

### 5-(aminomethyl)-1-(trans-3-hydroxycyclobutyl)-N,N-dimethyl-1H-pyrazole-3-carboxamide

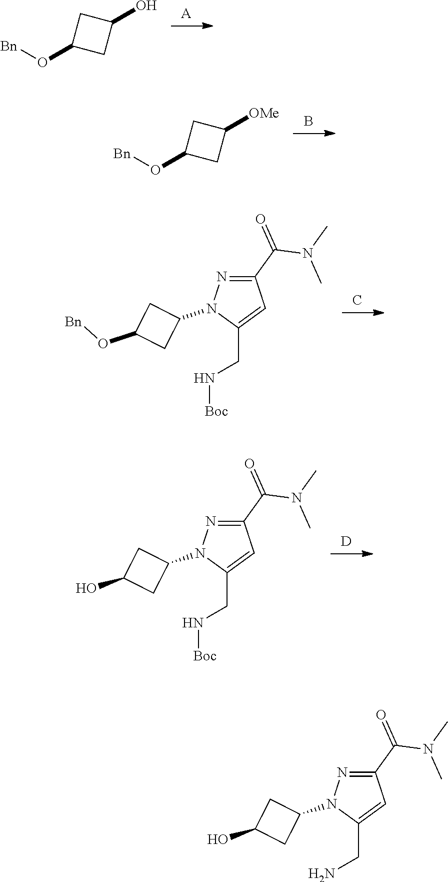

Step A. cis-3-(benzyloxy)cyclobutyl methanesulfonate: To a solution of cis-3-(benzyloxy)cyclobutan-1-ol (1.0 g, 1.0 equiv), DMAP (68.6 mg, 0.1 equiv) and TEA (1.7 g, 3.0 equiv) in DCM (10 mL) was added methylsulfonyl methanesulfonate (2.0 g, 11.2 mmol, 2.0 equiv) drop-wise at 0° C. under N₂. The reaction was stirred at 25° C. for 2 hours. The mixture was quenched by ice slowly and then extracted with DCM (20 mL×3). The combined organic phases were washed with brine (25 mL), dried over anhydrous Na₂SO₄, concentrated, and purified by column chromatography (SiO₂, Petroleum ether/Ethyl acetate=10/1 to 5/1) to afford the title compound (1.4 g, 97.4% yield) as yellow liquid. ¹H NMR (400 MHZ, CHLOROFORM-d) δ=7.39-7.28 (m, 5H), 4.66 (quin, J=7.2 Hz, 1H), 4.44 (s, 2H), 3.75 (quin, J=6.8 Hz, 1H), 2.99 (s, 3H), 2.89-2.79 (m, 2H), 2.39-2.30 (m, 2H).

Step B. tert-butyl ((1-(trans-3-(benzyloxy)cyclobutyl)-3-(dimethylcarbamoyl)-1H-pyrazol-5-yl)methyl)carbamate: To a mixture of tert-butyl N-[[3-(dimethylcarbamoyl)-1H-pyrazol-5-yl]methyl]carbamate (100 mg, 1.0 equiv) and cis-3-(benzyloxy)cyclobutyl methanesulfonate (143 mg, 1.5 equiv) in DMF (2 mL) was added Cs₂CO₃ (364 mg, 3.0 equiv) in one portion at 25° C. under N₂. The reaction was heated to 90° C. and stirred for 12 hours. The mixture was poured into ice-water (3 mL) and extracted with ethyl acetate (4 mL×3). The combined organic phases were washed with brine (5 mL), dried with anhydrous Na₂SO₄, filtered, and purified by prep-TLC (SiO₂, PE/EA=3:1) to afford the title compound (38 mg, 20% yield) as yellow oil. ¹H NMR (400 MHZ, CHLOROFORM-d) δ=7.28-7.21 (m, 5H), 6.49 (s, 1H), 4.93 (br s, 1H), 4.70 (brs, 1H), 4.42-4.39 (m, 3H), 4.26 (br d, J=5.6 Hz, 2H), 3.27 (s, 3H), 3.02 (s, 3H), 2.71-2.66 (m, 2H), 2.54-2.50 (m, 2H), 1.37 (s, 9H). The other regioisomer was also observed.

Step C. tert-butyl ((3-(dimethylcarbamoyl)-1-(trans-3-hydroxycyclobutyl)-1H-pyrazol-5-yl)methyl)carbamate: To a solution of tert-butyl ((1-(trans-3-(benzyloxy)cyclobutyl)-3-(dimethylcarbamoyl)-1H-pyrazol-5-yl)methyl)carbamate (175 mg, 1.0 equiv) in MeOH (3 mL) was added Pd/C (100 mg, 10% purity) under N₂. The suspension was degassed under vacuum and purged with H₂ several times. The reaction was stirred under H₂ (15 psi) at 25° C. for 1 hour. The mixture was filtered, and the filtrate was concentrated to afford the title compound (100 mg, crude) as white solid. ¹H NMR (400 MHZ, CHLOROFORM-d) δ=6.48 (s, 1H), 5.01-4.89 (m, 1H), 4.60 (br s, 1H), 4.22 (br s, 2H), 4.12-3.92 (m, 3H), 3.35 (s, 2H), 3.32-3.15 (m, 3H), 3.13-2.98 (m, 3H), 2.71 (td, J=6.0, 12.0 Hz, 2H), 2.47-2.32 (m, 2H), 1.36 (s, 9H).

Step D. 5-(aminomethyl)-1-(trans-3-hydroxycyclobutyl)-N,N-dimethyl-1H-pyrazole-3-carboxamide: To a solution of tert-butyl ((3-(dimethylcarbamoyl)-1-(trans-3-hydroxycyclobutyl)-1H-pyrazol-5-yl)methyl)carbamate (100 mg, 1.0 equiv) in dioxane (1 mL) was added HCl/dioxane (4 M, 4.0 equiv) at 25° C. The reaction was stirred at 25° C. for 1 hour. The mixture was concentrated to give the crude product (70 mg, crude, HCl) as yellow solid. LCMS (ESI, M+1): m/z=239.2.

### Intermediate 37

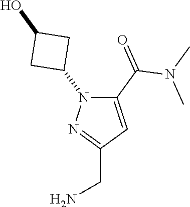

### 3-(aminomethyl)-1-(trans-3-hydroxycyclobutyl)-N,N-dimethyl-1H-pyrazole-5-carboxamide

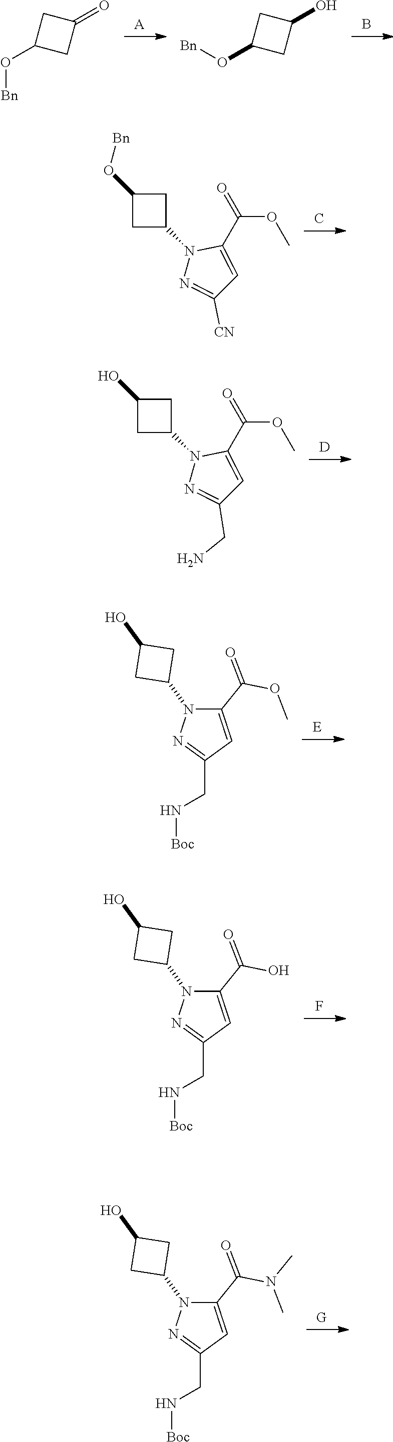

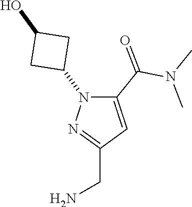 [SMILES: CN(C)C(=O)c1cc(CN)nn1[C@H]1C[C@H](O)C1]

Step A. cis-3-(benzyloxy)cyclobutanol: To a solution of 3-(benzyloxy)cyclobutanone (7.00 g, 1.0 equiv) in MeOH (120 mL) was added NaBH₄ (1.80 g, 1.2 equiv) in portions at 0° C. under nitrogen atmosphere. The reaction was stirred at 0° C. for 1 hour. The mixture was quenched with saturated ammonium chloride solution (300 mL) slowly at 0-5° C., concentrated to remove MeOH, and extracted with EtOAc (70 mL×3). The combined organic layers were washed with brine (100 mL), dried over anhydrous sodium sulfate and concentrated to afford the title compound (6.88 g, 94% yield) as colorless liquid; ¹H NMR (400 MHZ, CHLOROFORM-d) δ=7.39-7.29 (m, 5H), 4.49-4.38 (m, 2H), 3.96-3.87 (m, 1H), 3.64 (t, J=6.8 Hz, 1H), 2.76-2.68 (m, 2H), 1.98-1.92 (m, 2H).

Step B. methyl 1-(trans-3-(benzyloxy)cyclobutyl)-3-cyano-1H-pyrazole-5-carboxylate: To a mixture of methyl 5-cyano-1H-pyrazole-3-carboxylate (1.80 g, 1.0 equiv), cis-3-(benzyloxy)cyclobutanol (2.12 g, 1.0 equiv) and PPh₃ (6.25 g, 2.0 equiv) in THF (40 mL) was added DIAD (4.82 g, 4.6 mL, 2.0 equiv). The reaction was stirred at 0° C. for 0.5 hours and 25° C. for 12 hours. The mixture was concentrated and purified with flash silica gel chromatography (ISCO®; 80 g Sepaflash® silica flash column, eluent of 30% ethyl acetate/petroleum ether gradient @ 100 mL/min) to afford the title compound (3.00 g, 71% yield) as yellow oil; 1H NMR (400 MHZ, CHLOROFORM-d) o=7.37 (d, J=4.4 Hz, 4H), 7.34-7.29 (m, 1H), 7.20 (s, 1H), 5.95-5.78 (m, 1H), 4.49 (s, 2H), 4.45 (ddd, J=2.4, 4.4, 6.8 Hz, 1H), 3.91 (s, 3H), 2.88-2.76 (m, 2H), 2.74-2.62 (m, 2H).

Step C. methyl 3-(aminomethyl)-1-(trans-3-hydroxycyclobutyl)-1H-pyrazole-5-carboxylate: To a solution of methyl 1-(trans-3-(benzyloxy)cyclobutyl)-3-cyano-1H-pyrazole-5-carboxylate (600 mg, 1.0 equiv) in MeOH (20 mL) and HCl solution (1 mL, 2 M in MeOH) was added Pd/C (100 mg, 10% purity, wet). The reaction was degassed and purged with H₂ 3 times. The reaction was stirred at 25° C. under H₂ atmosphere (15 psi) for 2 hours. The mixture was filtered and concentrated to afford the title compound (500 mg, 99% yield, HCl) as white solid.

Step D. methyl 3-(((tert-butoxycarbonyl)amino)methyl)-1-(trans-3-hydroxycyclobutyl)-1H-pyrazole-5-carboxylate: To a mixture of methyl 3-(aminomethyl)-1-(trans-3-hydroxycyclobutyl)-1H-pyrazole-5-carboxylate (500 mg, 1.0 equiv, HCl) and TEA (580 mg, 0.8 mL, 3.0 equiv) in dichloromethane (10 mL) was added BoczO (625 mg, 0.6 mL, 1.5 equiv). The reaction was stirred at 30° C. for 2 hours. The mixture was concentrated and purified with flash silica gel chromatography (ISCO®; 40 g Sepaflash® silica flash column, eluent of 50-70% EtOAc/PE gradient @ 50 mL/min) to afford the title compound (200 mg, 31% yield, 95% purity) as white solid; ¹H NMR (400 MHZ, CHLOROFORM-d) δ=6.76 (s, 1H), 5.82 (br s, 1H), 5.06-4.92 (m, 1H), 4.81-4.69 (m, 1H), 4.34 (br d, J=4.4 Hz, 2H), 3.86 (s, 3H), 2.93-2.81 (m, 2H), 2.50 (ddd, J=4.0, 8.4, 13.2 Hz, 2H), 1.48 (s, 9H); LCMS (ESI, M+1): m/z=326.1.

Step E: 3-(((tert-butoxycarbonyl)amino)methyl)-1-(trans-3-hydroxycyclobutyl)-1H-pyrazole-5-carboxylic acid: To a solution of methyl 3-(((tert-butoxycarbonyl)amino)methyl)-1-(trans-3-hydroxycyclobutyl)-1H-pyrazole-5-carboxylate (200 mg, 1 equiv) in MeOH (2 mL) and H₂O (1 mL) was added KOH (51.7 mg, 1.5 equiv). The mixture was stirred at 40° C. for 2 hours. The reaction was quenched by ice slowly and then extracted with DCM (5 mL×3). The combined organic phase was washed with brine (10 mL), dried over anhydrous Na₂SO₄, filtered and concentrated to afford the title compound (166 mg, crude) as white solid; 1H NMR (400 MHZ, DMSO-d6) δ=13.43-13.08 (m, 1H), 7.29 (br t, J=5.6 Hz, 1H), 6.63 (s, 1H), 5.76-5.66 (m, 1H), 5.15 (br d, J=4.4 Hz, 1H), 4.40 (br d, J=3.6 Hz, 1H), 4.09 (br d, J=5.6 Hz, 2H), 2.70-2.57 (m, 2H), 2.29 (ddd, J=4.0, 8.5, 12.6 Hz, 2H), 1.39 (s, 9H).

Step F. tert-butyl ((5-(dimethylcarbamoyl)-1-(trans-3-hydroxycyclobutyl)-1H-pyrazol-3-yl)methyl)carbamate: To a solution of 3-(((tert-butoxycarbonyl)amino)methyl)-1-(trans-3-hydroxycyclobutyl)-1H-pyrazole-5-carboxylic acid (143 mg, 1.0 equiv) and N-methylmethanamine (93.6 mg, 2.5 equiv, HCl) in DMF (2 mL) were added DIEA (297 mg, 5.0 equiv) and HATU (524 mg, 3.0 equiv). The reaction was stirred at 25° C. for 12 hours. The residue was poured into ice-water (5 mL) and extracted with ethyl acetate (5 mL×3). The combined organic phases were washed with brine (5 mL), dried with anhydrous Na₂SO₄, filtered, and concentrated in vacuum to afford the tittle compound (155 mg, 94.7% yield,) as yellow liquid.

Step G. 3-(aminomethyl)-1-(trans-3-hydroxycyclobutyl)-N,N-dimethyl-1H-pyrazole-5-carboxamide: To a solution of tert-butyl ((5-(dimethylcarbamoyl)-1-(trans-3-hydroxycyclobutyl)-1H-pyrazol-3-yl)methyl)carbamate (155 mg, 1.0 equiv) in dioxane (1 mL) was added HCl/dioxane (4 M, 1 mL). The reaction was stirred at 25° C. for 2 hours. The mixture was concentrated to afford the title compound (100 mg, crude, HCl) as yellow solid. LCMS (ESI, M+1): m/z=239.0.

### Example 524

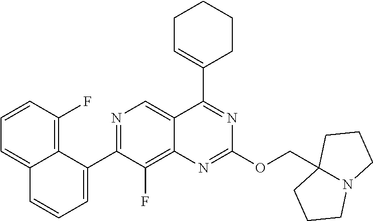 [SMILES: Fc1c(-c2cccc3cccc(F)c23)ncc2c(C3=CCCCC3)nc(OCC34CCCN3CCC4)nc12]

### 4-(cyclohex-1-en-1-yl)-8-fluoro-7-(8-fluoronaphthalen-1-yl)-2-((hexahydro-1H-pyrrolizin-7a-yl)methoxy)pyrido[4,3-d]pyrimidine

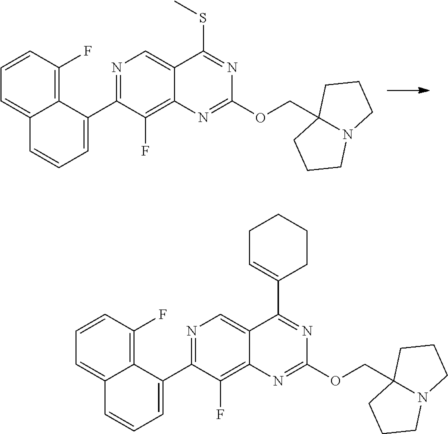 [SMILES: CSc1nc(OCC23CCCN2CCC3)nc2c(F)c(-c3cccc4cccc(F)c34)ncc12 ; Fc1c(-c2cccc3cccc(F)c23)ncc2c(C3=CCCCC3)nc(OCC34CCCN3CCC4)nc12]

A mixture of Intermediate 1 (60 mg, 1 equiv), tributyl(cyclohexen-1-yl) stannane (93.1 mg, 2 equiv), cuprous; 2-hydroxy-3-methyl-benzoate (80.8 mg, 3 equiv), Pd(PPh₃)₄ (14.5 mg, 0.1 equiv) in THF (2 mL) was degassed and purged with N₂ for 3 times. The mixture was stirred at 60° C. for 15 hours under N₂ atmosphere. The reaction mixture was diluted with water (20 mL) and extracted with ethyl acetate (20 mL×3). The combined organic layers were washed with brine (20 mL), dried over sodium sulfate, filtered, concentrated and purified by prep-HPLC (column: Phenomenex Luna C18 150×25 mm×10 um; mobile phase: [water (FA)-ACN]; B %: 23%-53%, 10 min) to afford the title compound (1.27 mg, 2.45 μmol, 1.95% yield) as an off-white solid (0.23 formic acid salt). 1H NMR (400 MHZ, METHANOL-d₄) δ=9.27 (s, 1H), 8.67-8.47 (m, 1H), 8.14 (br d, J=8.0 Hz, 1H), 7.87 (d, J=8.0 Hz, 1H), 7.74-7.69 (m, 1H), 7.65-7.62 (m, 1H), 7.54 (dt, J=5.0, 8.0 Hz, 1H), 7.20 (dd, J=7.0, 13.2 Hz, 1H), 6.49 (br t, J=3.6 Hz, 1H), 4.45 (s, 2H), 3.19 (br dd, J=5.8, 10.8 Hz, 2H), 2.81 (td, J=6.5, 10.8 Hz, 2H), 2.70 (br s, 2H), 2.51-2.39 (m, 2H), 2.13 (br dd, J=6.5, 12.4 Hz, 2H), 2.00-1.81 (m, 10H). LCMS (ESI, M+1): m/z=513.3.

### Example 525

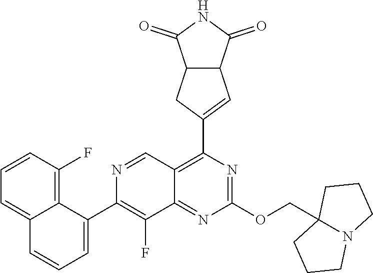 [SMILES: O=C1NC(=O)C2CC(c3nc(OCC45CCCN4CCC5)nc4c(F)c(-c5cccc6cccc(F)c56)ncc34)=CC12]

### 5-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-4,6a-dihydrocyclopenta[c]pyrrole-1,3(2H,3aH)-dione

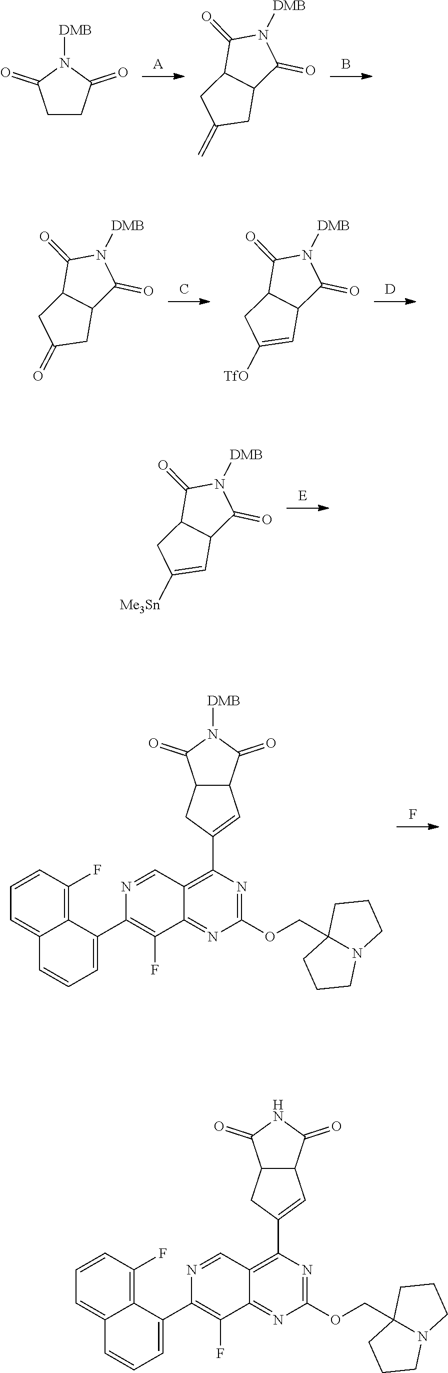

Step A. 2-[(2,4-dimethoxyphenyl)methyl]-5-methylene-3a,4,6,6a-tetrahydrocyclopenta[c]pyrrole-1,3-dione: To a solution of 1-[(2,4-dimethoxyphenyl)methyl]pyrrolidine-2,5-dione (12.0 g, 1.0 equiv) in THF (200 mL) was added LDA (2 M, 144 mL, 6.0 equiv) at −78° C. under nitrogen atmosphere. The mixture was stirred at −78° C. for 15 min and stirred at 0° C. for 1 hour. The mixture was cooled to −78° C. and a solution of 3-chloro-2-(chloromethyl) prop-1-ene (24.1 g, 4.0 equiv) in THF (100 mL) was added dropwise. The mixture was allowed to warm to 20° C. and stirred at 20° C. for 24 hours. The mixture was quenched with saturated ammonium chloride solution (500 mL) and water (500 mL). The mixture was extracted with EtOAc (300 mL×2). The organic layers were washed with brine (300 mL), dried over anhydrous sodium sulfate, concentrated and purified by column chromatography (SiO₂, petroleum ether/ethyl acetate 10:1 to 1:1) to afford the title compound (5.50 g, 21% yield, 55% purity) as a light yellow solid; ¹H NMR (400 MHZ, CHLOROFORM-d) δ=6.97 (d, J=8.4 Hz, 1H), 6.44-6.34 (m, 2H), 4.93 (s, 2H), 4.61 (s, 2H), 3.79 (s, 3H), 3.78 (s, 3H), 3.28-3.20 (m, 2H), 2.79-2.69 (m, 2H), 2.69-2.61 (m, 2H)

Step B. 2-[(2,4-dimethoxyphenyl)methyl]-3a,4,6,6a-tetrahydrocyclopenta[c]pyrrole-1,3,5-trione: To a solution of 2-[(2,4-dimethoxyphenyl)methyl]-5-methylene-3a,4,6,6a-tetrahydrocyclopenta[c]pyrrole-1,3-dione (2.00 g, 1.0 equiv) in THF (20 mL) and H₂O (20 mL) was added NaIO₄ (5.68 g, 4.0 equiv) and K₂OsO₄·2H₂O (122 mg, 0.05 equiv). The mixture was stirred at 20° C. for 3 hours. The mixture was diluted with water (200 mL) and extracted with EtOAc (50 mL×3). The combined organic layers were washed with brine (100 mL), dried over anhydrous sodium sulfate and concentrated and purified by column chromatography (SiO₂, petroleum ether/ethyl acetate 20:1 to 1:1) to afford the title compound (850 mg, 42% yield) as a light yellow oil; ¹H NMR (400 MHZ, CHLOROFORM-d) δ=7.14 (d, J=8.0 Hz, 1H), 6.46-6.41 (m, 2H), 4.66 (s, 2H), 3.79 (s, 3H), 3.77 (s, 3H), 3.56-3.49 (m, 2H), 2.84-2.73 (m, 2H), 2.62-2.51 (m, 2H)

Step C. [2-[(2,4-dimethoxyphenyl)methyl]-1,3-dioxo-6,6a-dihydro-3aH-cyclopenta[c]pyrrol-5-yl]trifluoromethanesulfonate: To a solution of 2-[(2,4-dimethoxyphenyl)methyl]-3a,4,6,6a-tetrahydrocyclopenta[c]pyrrole-1,3,5-trione (850 mg, 1.0 equiv) in THF (12 mL) was added LDA (2 M, 1.7 mL, 1.2 equiv) at −78° C. under nitrogen atmosphere. The mixture was stirred at −78° C. for 0.5 hour. A solution of 1,1,1-trifluoro-N-phenyl-N-(trifluoromethylsulfonyl)methanesulfonamide (1.00 g, 1.0 equiv) in THF (6 mL) was added. The reaction mixture was stirred at 20° C. for 16 hours. The mixture was quenched with saturated ammonium chloride solution (100 mL) and extracted with EtOAc (30 mL). The organic layer was dried over anhydrous sodium sulfate, concentrated and purified by column chromatography (SiO₂, petroleum ether/ethyl acetate 20:1 to 3:1) to afford the title compound (410 mg, 34% yield) as a light yellow oil; ¹H NMR (400 MHZ, CHLOROFORM-d) δ=7.04 (d, J=9.2 Hz, 1H), 6.44-6.39 (m, 2H), 5.81 (d, J=2.0 Hz, 1H), 4.73-4.57 (m, 2H), 3.89 (qd, J=2.8, 8.1 Hz, 1H), 3.79 (s, 6H), 3.46 (ddd, J=2.8, 7.8, 10.5 Hz, 1H), 3.16-3.06 (m, 1H), 3.03-2.93 (m, 1H)

Step D. 2-[(2,4-dimethoxyphenyl)methyl]-5-trimethylstannyl-6,6a-dihydro-3aH-cyclopenta[c]pyrrole-1,3-dione: To a solution of [2-[(2,4-dimethoxyphenyl)methyl]-1,3-dioxo-6,6a-dihydro-3aH-cyclopenta[c]pyrrol-5-yl]trifluoromethanesulfonate (100 mg, 1.0 equiv) and trimethyl(trimethylstannyl) stannane (90.3 mg, 1.2 equiv) in THF (2 mL) were added LiCl (29.2 mg, 3.0 equiv) and Pd(PPh₃)₄ (53.1 mg, 0.2 equiv). The mixture was stirred at 60° C. for 16 hours under nitrogen atmosphere. The mixture was filtered and purified by column chromatography (SiO₂, petroleum ether/ethyl acetate 10:1 to 2:1) to afford the title compound (50.0 mg, 48% yield) as a light yellow oil; ¹H NMR (400 MHZ, CHLOROFORM-d) δ=6.93 (d, J=8.0 Hz, 1H), 6.43-6.35 (m, 2H), 5.86 (d, J=2.4 Hz, 1H), 4.59 (d, J=3.6 Hz, 2H), 3.95 (dt, J=2.4, 5.0 Hz, 1H), 3.78 (d, J=1.6 Hz, 6H), 3.50-3.41 (m, 1H), 2.97-2.91 (m, 2H), 0.18 (s, 9H)

Step E. 2-(3,4-dimethoxybenzyl)-5-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-4,6a-dihydrocyclopenta[c]pyrrole-1,3(2H,3aH)-dione: To a solution of Intermediate 1 (40.0 mg, 1.0 equiv) and 2-[(2,4-dimethoxyphenyl)methyl]-5-trimethylstannyl-6,6a-dihydro-3aH-cyclopenta[c]pyrrole-1,3-dione (48.9 mg, 1.3 equiv) in THF (1 mL) were added thiophene-2-carbonyloxy copper (23.9 mg, 1.5 equiv), Pd₂(dba) 3 (7.65 mg, 0.1 equiv) and tris(2-furyl)phosphine (5.82 mg, 0.3 equiv). The mixture was stirred at 60° C. for 12 hours under nitrogen atmosphere. The mixture was diluted with water (20 mL) and extracted with EtOAc (10 mL×2). The combined organic layers were washed with brine (20 mL), dried over anhydrous sodium sulfate, concentrated and purified by column chromatography (SiO₂, petroleum ether/ethyl acetate 10:1 to 0:1 and ethyl acetate/MeOH 10:1) to afford the title compound (50.0 mg, 76% yield) as a light yellow solid; ¹H NMR (400 MHz, CHLOROFORM-d) δ=9.23 (d, J=2.8 Hz, 1H), 8.03 (br d, J=8.4 Hz, 1H), 7.77 (d, J=8.0 Hz, 1H), 7.69-7.63 (m, 1H), 7.62-7.57 (m, 1H), 7.47 (dt, J=5.2, 8.0 Hz, 1H), 7.17-7.08 (m, 2H), 6.66 (br s, 1H), 6.46-6.38 (m, 2H), 4.68 (s, 2H), 4.54-4.31 (m, 2H), 4.29-4.22 (m, 1H), 3.82-3.77 (m, 3H), 3.71 (d, J=16.0 Hz, 4H), 3.67-3.59 (m, 2H), 3.58-3.48 (m, 1H), 3.36-3.02 (m, 2H), 2.86-2.59 (m, 3H), 2.22-2.09 (m, 2H), 2.00-1.86 (m, 4H), 1.82-1.68 (m, 2H);

Step F. 5-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-4,6a-dihydrocyclopenta[c]pyrrole-1,3(2H,3aH)-dione: To a solution of 2-(3,4-dimethoxybenzyl)-5-(8-fluoro-7-(8-fluoronaphthalen-1-yl)-2-((tetrahydro-1H-pyrrolizin-7a(5H)-yl)methoxy)pyrido[4,3-d]pyrimidin-4-yl)-4,6a-dihydrocyclopenta[c]pyrrole-1,3(2H,3aH)-dione (40.0 mg, 1.0 equiv) in ACN (1 mL) was added a solution of CAN (153 mg, 5.0 equiv) in H₂O (1 mL) at 0° C. The mixture was stirred at 20° C. for 12 hours. The mixture was diluted with water (30 mL) and extracted with EtOAc (10 mL×5). The combined organic layers were washed with brine (30 mL), dried over anhydrous sodium sulfate and concentrated and purified by column chromatography (SiO₂, petroleum ether/ethyl acetate 10:1 to 0:1 and ethyl acetate/MeOH 10:1) and prep-HPLC [Phenomenex luna C18 150×25 mm×10 um; A: water (FA), B: ACN, B %: 12%-42% over 10 min] to afford the title compound (0.80 mg, 2.2% yield) as a yellow solid (0.40 formic acid salt); ¹H NMR (400 MHZ, CHLOROFORM-d) δ=9.34 (s, 1H), 8.30-8.23 (m, 1H), 8.03 (br d, J=8.4 Hz, 1H), 7.77 (d, J=8.0 Hz, 1H), 7.69-7.64 (m, 1H), 7.63-7.59 (m, 1H), 7.47 (dt, J=5.2, 8.0 Hz, 1H), 7.18-7.09 (m, 1H), 6.73 (br s, 1H), 4.95-4.85 (m, 1H), 4.84-4.75 (m, 1H), 4.39-4.26 (m, 1H), 3.94-3.82 (m, 2H), 3.75-3.67 (m, 1H), 3.67-3.54 (m, 2H), 3.04-2.91 (m, 2H), 2.41 (br dd, J=6.0, 13.6 Hz, 2H), 2.33-2.26 (m, 2H), 2.12 (br dd, J=6.4, 12.8 Hz, 2H), 2.01 (br dd, J=6.4, 13.2 Hz, 2H); LCMS (ESI, M+1): m/z=568.2.

### Example 526

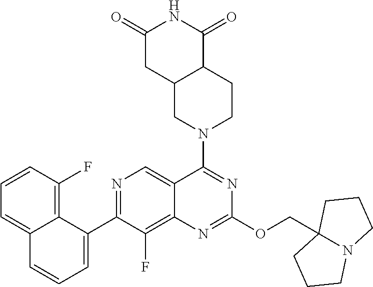 [SMILES: O=C1CC2CN(c3nc(OCC45CCCN4CCC5)nc4c(F)c(-c5cccc6cccc(F)c56)ncc34)CCC2C(=O)N1]

### 6-(8-fluoro-7-(8-fluoronaphthalen-1-yl)-2-((hexahydro-1H-pyrrolizin-7a-yl)methoxy)pyrido[4,3-d]pyrimidin-4-yl)hexahydro-2,6-naphthyridine-1,3(2H,4H)-dione

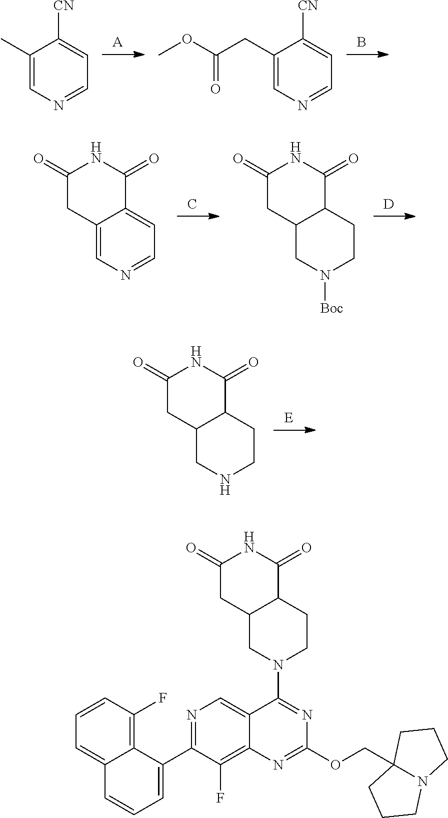

Step A. methyl 2-(4-cyanopyridin-3-yl)acetate: To a mixture of 3-methylisonicotinonitrile (2.2 g, 1.0 equiv) and dimethyl carbonate (2.52 g, 1.5 equiv) in THF (20 mL) was added dropwise KHMDS (1 M, 37.2 mL, 2.0 equiv) at −60° C. The reaction was stirred at −60° C. for 0.5 hour. The mixture was quenched with saturated NH₄Cl solution (50 ml) and extracted with EtOAc (100 mL). The organic layer was dried over anhydrous sodium sulfate, concentrated and purified by column chromatography (SiO₂, petroleum ether/ethyl acetate 20:1 to 2:1) to afford the title compound (600 mg, 17% yield) as a yellow oil; LCMS (ESI, M+1): m/z=177.1.

Step B. 2,6-naphthyridine-1,3(2H,4H)-dione: To a mixture of methyl 2-(4-cyanopyridin-3-yl)acetate (300 mg, 1.0 equiv) in toluene (6 mL) were added tris(triphenylphosphine) rhodium (I) chloride (78.8 mg, 0.05 equiv) and (E)-acetaldehyde oxime (503 mg, 5 equiv). The mixture was degassed and purged with nitrogen 3 times. The reaction was stirred at 110° C. for 6 hours under nitrogen atmosphere. The mixture was concentrated and triturated with methanol (5 ml) at 25° C. for 1 hour to afford the title compound (170 mg, 45% yield) as a brown solid; LCMS (ESI, M+1): m/z=163.0.

Step C. tert-butyl 5,7-dioxooctahydro-2,6-naphthyridine-2(1H)-carboxylate: To a mixture of 2,6-naphthyridine-1,3(2H,4H)-dione (170 mg, 1.0 equiv) and (Boc)₂O (333 mg, 2.0 equiv) in MeOH (10 mL) was added Pd/C (30 mg, 10% purity). The reaction was degassed and purged with hydrogen 3 times. The reaction was stirred at 40° C. for 20 hours under H₂ (50 psi) atmosphere. The mixture was filtered and concentrated to afford the title compound (147 mg, 45% yield) as a brown solid, LCMS (ESI, M−55): m/z=213.0.

Step D. hexahydro-2,6-naphthyridine-1,3(2H,4H)-dione: To a mixture of tert-butyl 5,7-dioxooctahydro-2,6-naphthyridine-2(1H)-carboxylate (147 mg, 1.0 equiv) in dichloromethane (2 mL) was added TFA (770 mg, 12 equiv). The reaction was stirred at 25° C. for 0.5 hour. The mixture was concentrated to afford the title compound (160 mg, crude, TFA salt) as a brown oil.

Step E. 6-(8-fluoro-7-(8-fluoronaphthalen-1-yl)-2-((hexahydro-1H-pyrrolizin-7a-yl)methoxy)pyrido[4,3-d]pyrimidin-4-yl)hexahydro-2,6-naphthyridine-1,3(2H,4H)-dione: To a mixture of hexahydro-2,6-naphthyridine-1,3(2H,4H)-dione (160 mg, 5.0 equiv, TFA) and 8-fluoro-7-(8-fluoronaphthalen-1-yl)-2-((hexahydro-1H-pyrrolizin-7a-yl)methoxy)-4-(2,2,2-trifluoroethoxy)pyrido[4,3-d]pyrimidine (60 mg, 1.0 equiv) in DMF (0.05 mL) were added DIPEA (223 mg, 15 equiv) and 4 Å molecular sieves (50 mg). The reaction was stirred at 40° C. for 16 hours. The mixture was filtered and purified with prep-HPLC [Phenomenex Luna C18 75×30 mm×3 μm; A: water (FA), B: ACN, B %: 5%-35% over 10 min] and lyophilized to afford the title compound (7.54 mg, 9.3% yield over two steps,) as a brown solid (0.95 formic acid salt); ¹H NMR (400 MHZ, methanol-d₄) δ=9.18-9.10 (m, 1H), 8.18-8.11 (m, 1H), 7.91-7.85 (m, 1H), 7.76-7.69 (m, 1H), 7.67-7.60 (m, 1H), 7.59-7.52 (m, 1H), 7.27-7.16 (m, 1H), 4.67 (s, 2H), 4.30-3.89 (m, 3H), 3.74-3.62 (m, 2H), 3.32-3.06 (m, 4H), 2.92-2.78 (m, 1H), 2.77-2.42 (m, 3H), 2.40-2.31 (m, 2H), 2.30-2.15 (m, 4H), 2.12 (br dd, J=6.4, 12.4 Hz, 3H); LCMS (ESI, M+1): m/z=599.2.

### Example 527

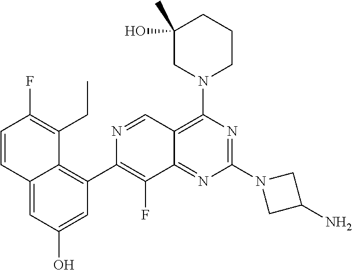 [SMILES: CCc1c(F)ccc2cc(O)cc(-c3ncc4c(N5CCC[C@@](C)(O)C5)nc(N5CC(N)C5)nc4c3F)c12]

### (R)-1-(2-(3-aminoazetidin-1-yl)-7-(8-ethyl-7-fluoro-3-hydroxynaphthalen-1-yl)-8-fluoropyrido[4,3-d]pyrimidin-4-yl)-3-methylpiperidin-3-ol

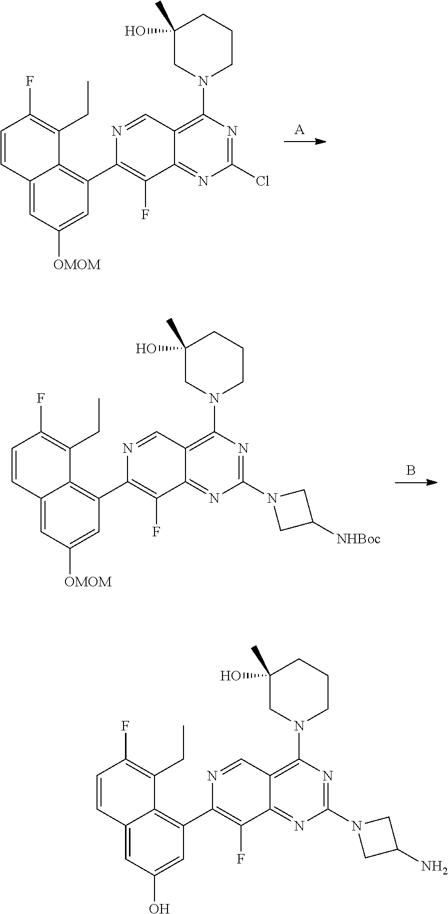

Step A. (R)-tert-butyl (1-(7-(8-ethyl-7-fluoro-3-(methoxymethoxy)naphthalen-1-yl)-8-fluoro-4-(3-hydroxy-3-methylpiperidin-1-yl)pyrido[4,3-d]pyrimidin-2-yl) azetidin-3-yl)carbamate: To a mixture of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (200 mg, 1.0 equiv), tert-butyl N-(azetidin-3-yl)carbamate (130 mg, 2.0 equiv) in dioxane (2 mL) was added DIPEA (244 mg, 5.0 equiv), and then the mixture was stirred at 90° C. for 12 hours under N₂ atmosphere. The reaction mixture was diluted (40 mL) and extracted with ethyl acetate (2×30 mL). The combined organic layers were washed with brine (40 mL), dried over sodium sulfate and concentrated to afford the title compound (200 mg, 80% yield) as a yellow solid; LCMS (ESI, M+1): m/z=665.3.

Step B. (R)-1-(2-(3-aminoazetidin-1-yl)-7-(8-ethyl-7-fluoro-3-hydroxynaphthalen-1-yl)-8-fluoropyrido[4,3-d]pyrimidin-4-yl)-3-methylpiperidin-3-ol: To a solution of tert-butyl N-[1-[7-[8-ethyl-7-fluoro-3-(methoxymethoxy)-1-naphthyl]-8-fluoro-4-[(3R)-3-hydroxy-3-methyl-1-piperidyl]pyrido[4,3-d]pyrimidin-2-yl]azetidin-3-yl]carbamate (150 mg, 1.0 equiv) in dioxane (1.5 mL) was added HCl/dioxane (4 M, 564 μL, 10.0 equiv) at 0° C. The mixture was stirred at 0° C. for 2 hours. The reaction was quenched with saturated sodium bicarbonate (20 mL). The mixture was extracted with ethyl acetate (3×30 mL). The combined organic layers were washed with brine (20 mL), dried over sodium sulfate, concentrated and purified with prep-HPLC (C18, A: water [(0.1% FA)-ACN]; B: ACN, B %: 45%-65%, over 25 min) to afford the title compound (96.3 mg, 82% yield) as a white solid (0.52 formic acid salt); 1H NMR (400 MHZ, METHANOL-d4) δ=8.98 (d, J=1.6 Hz, 1H), 8.51 (br s, 1H), 7.67 (dd, J=6.0, 9.2 Hz, 1H), 7.29 (d, J=2.4 Hz, 1H), 7.24 (t, J=9.2 Hz, 1H), 7.04 (s, 1H), 4.58-4.48 (m, 2H), 4.31 (br d, J=13.2 Hz, 1H), 4.18-4.01 (m, 4H), 3.64-3.50 (m, 1H), 3.47-3.39 (m, 1H), 2.59-2.42 (m, 1H), 2.33-2.19 (m, 1H), 2.18-2.05 (m, 1H), 1.89-1.68 (m, 3H), 1.26 (d, J=13.6 Hz, 3H), 0.82 (q, J=7.2 Hz, 3H); LCMS (ESI, M+1): m/z=521

### Example 528

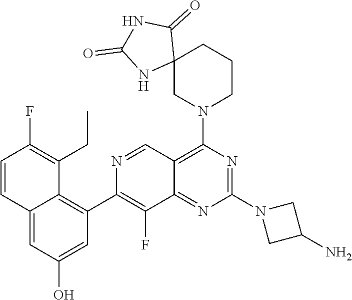 [SMILES: CCc1c(F)ccc2cc(O)cc(-c3ncc4c(N5CCCC6(C5)NC(=O)NC6=O)nc(N5CC(N)C5)nc4c3F)c12]

### 7-(2-(3-aminoazetidin-1-yl)-7-(8-ethyl-7-fluoro-3-hydroxynaphthalen-1-yl)-8-fluoropyrido[4,3-d]pyrimidin-4-yl)-1,3,7-triazaspiro[4.5]decane-2,4-dione

The title compound was synthesized from 7-(2-chloro-7-(8-ethyl-7-fluoro-3-(methoxymethoxy)naphthalen-1-yl)-8-fluoropyrido[4,3-d]pyrimidin-4-yl)-1,3,7-triazaspiro[4.5]decane-2,4-dione according to the 2-step procedure described for example 527 as a white solid. (0.54 formic acid salt) ¹H NMR (400 MHZ, DMSO-d₆) δ=8.87 (d, J=1.2 Hz, 1H), 8.68 (d, J=2.0 Hz, 1H), 7.74 (dd, J=6.0, 9.2 Hz, 1H), 7.39-7.27 (m, 2H), 6.99 (dd, J=2.4, 6.0 Hz, 1H), 4.35-4.16 (m, 4H), 3.93-3.69 (m, 4H), 3.55-3.25 (m, 3H), 2.39-2.30 (m, 1H), 2.25-2.12 (m, 1H), 2.10-1.96 (m, 2H), 1.94-1.77 (m, 2H), 0.81-0.68 (m, 3H); LCMS (ESI, M+1): m/z=575.3.

### Example 529

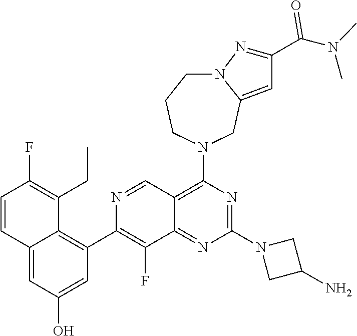 [SMILES: CCc1c(F)ccc2cc(O)cc(-c3ncc4c(N5CCCn6nc(C(=O)N(C)C)cc6C5)nc(N5CC(N)C5)nc4c3F)c12]

### 5-(2-(3-aminoazetidin-1-yl)-7-(8-ethyl-7-fluoro-3-hydroxynaphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide according to the 2-step procedure described for example 527 as a white solid. (0.40 formic acid salt) ¹H NMR (400 MHZ, METHANOL-d₄) δ=8.95 (s, 1H), 7.66 (dd, J=6.0, 9.2 Hz, 1H), 7.31-7.19 (m, 2H), 7.04 (d, J=2.4 Hz, 1H), 6.65 (s, 1H), 5.22-5.01 (m, 2H), 4.57-4.43 (m, 4H), 4.33 (td, J=5.6, 14.4 Hz, 2H), 4.15-4.06 (m, 3H), 3.32 (s, 3H), 2.56-2.44 (m, 1H), 2.34 (br s, 2H), 2.28-2.14 (m, 1H), 0.79 (t, J=7.2 Hz, 3H); LCMS (ESI, M+1): m/z=614.4.

### Example 530

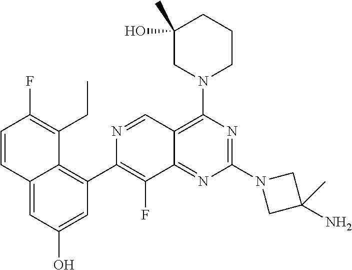 [SMILES: CCc1c(F)ccc2cc(O)cc(-c3ncc4c(N5CCC[C@@](C)(O)C5)nc(N5CC(C)(N)C5)nc4c3F)c12]

### (R)-1-(2-(3-amino-3-methylazetidin-1-yl)-7-(8-ethyl-7-fluoro-3-hydroxynaphthalen-1-yl)-8-fluoropyrido[4,3-d]pyrimidin-4-yl)-3-methylpiperidin-3-ol

The title compound was synthesized from (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol and tert-butyl N-(3-methylazetidin-3-yl)carbamate according to the 2-step procedure described for example 527 except for HCl·MeOH was used in Step B to produce the desired compound as as a white solid. (0.37 formic acid salt) ¹H NMR (400 MHZ, METHANOL-d4) δ=8.99 (d, J=2.0 Hz, 1H), 7.67 (dd, J=6.0, 9.2 Hz, 1H), 7.29 (d, J=2.4 Hz, 1H), 7.27-7.20 (m, 1H), 7.03 (s, 1H), 4.32 (br d, J=13.2 Hz, 1H), 4.18 (s, 4H), 4.08 (br dd, J=8.4, 13.2 Hz, 1H), 3.46 (br s, 2H), 2.50 (ddd, J=2.0, 7.2, 14.4 Hz, 1H), 2.34-2.19 (m, 1H), 2.17-2.03 (m, 1H), 1.90-1.70 (m, 3H), 1.60 (s, 3H), 1.26 (d, J=13.2 Hz, 3H), 0.88-0.78 (m, 3H); LCMS (ESI, M+1): m/z=535.3.

### Example 531

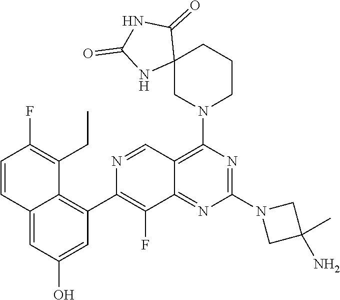 [SMILES: CCc1c(F)ccc2cc(O)cc(-c3ncc4c(N5CCCC6(C5)NC(=O)NC6=O)nc(N5CC(C)(N)C5)nc4c3F)c12]

### 7-(2-(3-amino-3-methylazetidin-1-yl)-7-(8-ethyl-7-fluoro-3-hydroxynaphthalen-1-yl)-8-fluoropyrido[4,3-d]pyrimidin-4-yl)-1,3,7-triazaspiro[4.5]decane-2,4-dione

The title compound was synthesized from 7-(2-chloro-7-(8-ethyl-7-fluoro-3-(methoxymethoxy)naphthalen-1-yl)-8-fluoropyrido[4,3-d]pyrimidin-4-yl)-1,3,7-triazaspiro[4.5]decane-2,4-dione and tert-butyl N-(3-methylazetidin-3-yl)carbamate according to the 2-step procedure described for example 527 to produce the desired compound as as a white solid. (0.7 formic acid salt) ¹H NMR (400 MHZ, DMSO-d₆) δ=8.87 (s, 1H), 8.25 (s, 1H), 7.74 (dd, J=6.0, 8.8 Hz, 1H), 7.39-7.26 (m, 2H), 6.99 (dd, J=2.4, 6.4 Hz, 1H), 4.39-4.15 (m, 3H), 3.97-3.89 (m, 4H), 3.51-3.29 (m, 3H), 2.31-1.96 (m, 4H), 1.93-1.76 (m, 2H), 1.41 (s, 3H), 0.81-0.67 (m, 3H); LCMS (ESI, M+1): m/z=589.3

### Example 532

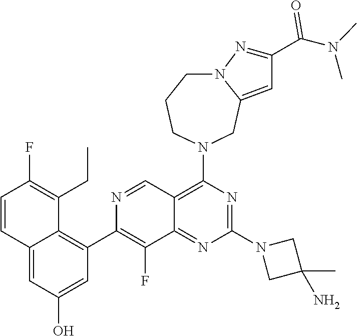 [SMILES: CCc1c(F)ccc2cc(O)cc(-c3ncc4c(N5CCCn6nc(C(=O)N(C)C)cc6C5)nc(N5CC(C)(N)C5)nc4c3F)c12]

### 5-(2-(3-amino-3-methylazetidin-1-yl)-7-(8-ethyl-7-fluoro-3-hydroxynaphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide and tert-butyl N-(3-methylazetidin-3-yl)carbamate according to the 2-step procedure described for example 527 to produce the desired compound as as a white solid. (0.31 formic acid salt). ¹H NMR (400 MHZ, METHANOL-d₄) δ=8.95 (s, 1H), 7.66 (dd, J=6.0, 9.2 Hz, 1H), 7.29 (d, J=2.4 Hz, 1H), 7.24 (t, J=9.2 Hz, 1H), 7.03 (d, J=2.4 Hz, 1H), 6.65 (s, 1H), 5.25-5.03 (m, 2H), 4.56-4.48 (m, 2H), 4.35 (br d, J=15.6 Hz, 2H), 4.12 (s, 4H), 3.33 (s, 3H), 3.32-3.30 (m, 6H), 3.08 (s, 3H), 2.62-2.45 (m, 1H), 2.40-2.30 (m, 2H), 2.27-2.16 (m, 1H), 1.56 (s, 3H), 0.80 (t, J=7.2 Hz, 3H); LCMS (ESI, M+1): m/z=628.4.

### Example 533

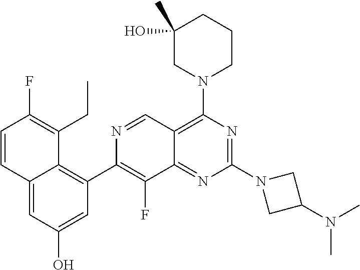 [SMILES: CCc1c(F)ccc2cc(O)cc(-c3ncc4c(N5CCC[C@@](C)(O)C5)nc(N5CC(N(C)C)C5)nc4c3F)c12]

### (R)-1-(2-(3-(dimethylamino) azetidin-1-yl)-7-(8-ethyl-7-fluoro-3-hydroxynaphthalen-1-yl)-8-fluoropyrido[4,3-d]pyrimidin-4-yl)-3-methylpiperidin-3-ol

The title compound was synthesized from (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol and N,N-dimethylazetidin-3-amine dihydrochloride according to the 2-step procedure described for example 527 to produce the desired compound as as a white solid. (0.28 formic acid salt) ¹H NMR (400 MHZ, METHANOL-d₄) δ=8.97 (d, J=2.4 Hz, 1H), 8.35 (br s, 1H), 7.66 (dd, J=5.9, 9.0 Hz, 1H), 7.29 (d, J=2.6 Hz, 1H), 7.24 (t, J=9.4 Hz, 1H), 7.08-7.01 (m, 1H), 4.41-4.25 (m, 3H), 4.15-4.02 (m, 3H), 3.64-3.48 (m, 1H), 3.47-3.36 (m, 2H), 3.31 (s, 2H), 2.56-2.46 (m, 1H), 2.36 (s, 6H), 2.24 (dqd, J=4.6, 7.4, 14.5 Hz, 1H), 2.17-2.03 (m, 1H), 1.82 (br s, 1H), 1.79-1.69 (m, 2H), 1.26 (d, J=13.4 Hz, 3H), 0.89-0.77 (m, 3H); LCMS (ESI, M+1): m/z=: 549.2.

### Example 534

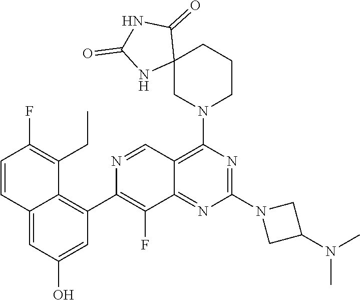 [SMILES: CCc1c(F)ccc2cc(O)cc(-c3ncc4c(N5CCCC6(C5)NC(=O)NC6=O)nc(N5CC(N(C)C)C5)nc4c3F)c12]

### 7-(2-(3-(dimethylamino) azetidin-1-yl)-7-(8-ethyl-7-fluoro-3-hydroxynaphthalen-1-yl)-8-fluoropyrido[4,3-d]pyrimidin-4-yl)-1,3,7-triazaspiro[4.5]decane-2,4-dione

The title compound was synthesized from 7-(2-chloro-7-(8-ethyl-7-fluoro-3-(methoxymethoxy)naphthalen-1-yl)-8-fluoropyrido[4,3-d]pyrimidin-4-yl)-1,3,7-triazaspiro[4.5]decane-2,4-dione and N,N-dimethylazetidin-3-amine dihydrochloride according to the 2-step procedure described for example 527 to produce the desired compound as a white solid. (0.32 formic acid salt) ¹H NMR (400 MHZ, DMSO-d₆) δ=10.87-10.62 (m, 1H), 10.17-9.53 (m, 1H), 8.87 (s, 1H), 8.66 (1H), 7.75 (dd, J=6.0, 9.2 Hz, 1H), 7.40-7.26 (m, 2H), 6.99 (dd, J=2.4, 6.0 Hz, 1H), 4.37-4.09 (m, 4H), 3.99-3.87 (m, 2H), 3.46-3.38 (m, 2H), 3.18-3.10 (m, 1H), 2.47-2.17 (m, 2H), 2.13 (s, 6H), 2.09-1.94 (m, 2H), 1.83 (br t, J=9.6 Hz, 2H), 0.80-0.68 (m, 3H); LCMS (ESI, M+1): m/z=603.4.

### Example 535

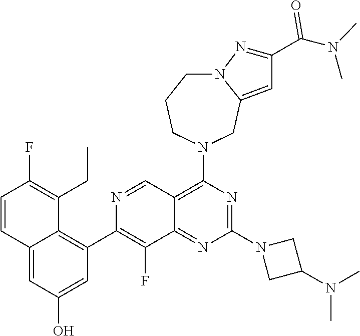 [SMILES: CCc1c(F)ccc2cc(O)cc(-c3ncc4c(N5CCCn6nc(C(=O)N(C)C)cc6C5)nc(N5CC(N(C)C)C5)nc4c3F)c12]

### 5-(2-(3-(dimethylamino) azetidin-1-yl)-7-(8-ethyl-7-fluoro-3-hydroxynaphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-5,6,7,8-tetrahydro-4H-pyrazolo[1,5-a][1,4]diazepine-2-carboxamide and N,N-dimethylazetidin-3-amine dihydrochloride according to the 2-step procedure described for example 527 to produce the desired compound as a yellow solid. ¹H NMR (400 MHZ, METHANOL-d₄) δ=8.94 (s, 1H), 7.66 (dd, J=6.0, 8.8 Hz, 1H), 7.29 (d, J=2.8 Hz, 1H), 7.24 (t, J=9.2 Hz, 1H), 7.03 (d, J=2.4 Hz, 1H), 6.69 (s, 1H), 5.28-5.02 (m, 2H), 4.52 (br d, J=5.6 Hz, 2H), 4.41-4.27 (m, 4H), 4.16-4.01 (m, 2H), 3.44-3.36 (m, 1H), 3.36-3.32 (m, 3H), 3.08 (s, 3H), 2.57-2.47 (m, 1H), 2.41-2.28 (m, 8H), 2.27-2.16 (m, 1H), 0.80 (t, J=7.2 Hz, 3H); LCMS (ESI, M+1): m/z=642.4.

### Example 536

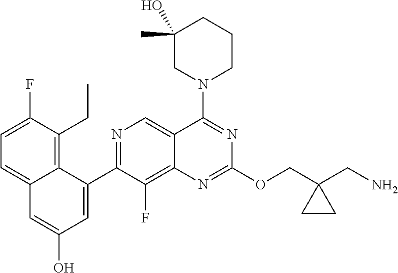 [SMILES: CCc1c(F)ccc2cc(O)cc(-c3ncc4c(N5CCC[C@@](C)(O)C5)nc(OCC5(CN)CC5)nc4c3F)c12]

### (R)-1-(2-((1-(aminomethyl)cyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

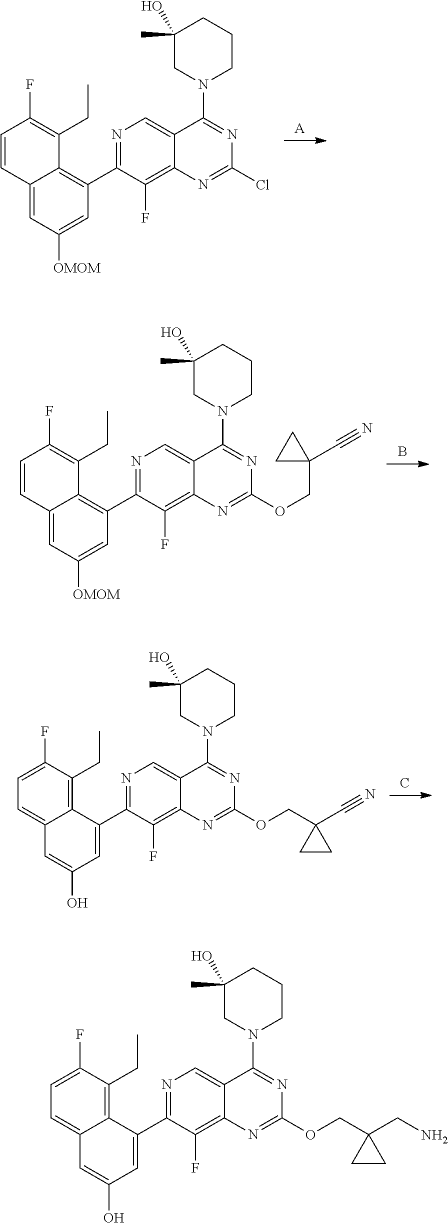

Step A. (R)-1-(((7-(8-ethyl-7-fluoro-3-(methoxymethoxy)naphthalen-1-yl)-8-fluoro-4-(3-hydroxy-3-methylpiperidin-1-yl)pyrido[4,3-d]pyrimidin-2-yl)oxy)methyl)cyclopropanecarbonitrile: To a solution of t-BuONa (2 M, 3.40 mL, 2.0 equiv) in THF was added 1-(hydroxymethyl)cyclopropanecarbonitrile (661 mg, 2.0 equiv). The reaction was stirred at 0° C. for 0.5 hour under N₂ atmosphere. The mixture was added into a solution of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (1.80 g, 1.0 equiv) in THF (10 mL) at 0° C. The reaction was stirred at 25° C. for 2 hours. The reaction mixture was quenched by addition of H₂O (10 mL) at 0° C. The mixture was extracted with EtOAc (3×20 mL). The combined organic layers were dried over anhydrous sodium sulfate, concentrated and purified by reversed phase flash chromatography [C18, 0.1% formic acid condition] to afford the title compound (900 mg, 45% yield) as a white solid; LCMS (ESI, M+1): m/z=590.2.

Step B. (R)-1-(((7-(8-ethyl-7-fluoro-3-hydroxynaphthalen-1-yl)-8-fluoro-4-(3-hydroxy-3-methylpiperidin-1-yl)pyrido[4,3-d]pyrimidin-2-yl)oxy)methyl)cyclopropanecarbonitrile: To a solution of (R)-1-(((7-(8-ethyl-7-fluoro-3-(methoxymethoxy)naphthalen-1-yl)-8-fluoro-4-(3-hydroxy-3-methylpiperidin-1-yl)pyrido[4,3-d]pyrimidin-2-yl)oxy)methyl)cyclopropanecarbonitrile (400 mg, 1.0 equiv) in DCM (4 mL) was added TFA (6.16 g, 4.00 mL) at 0° C. The mixture was stirred at 25° C. for 0.5 hour. The reaction mixture was quenched with saturated NaHCO₃ solution (8 mL) at 0° C. The mixture was extracted with EtOAc (3×5 mL). The combined organic layers were dried over anhydrous sodium sulfate, concentrated and purified by reversed phase flash chromatography [C18, 0.1% formic acid condition] to afford the title compound (15.4 mg, 11% yield) as a white solid; LCMS (ESI, M+1): m/z=546.3.

Step C. (R)-1-(2-((1-(aminomethyl)cyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of PtO₂ (83.2 mg, 1.0 equiv) in MeOH (2.5 mL) were added (R)-1-(((7-(8-ethyl-7-fluoro-3-hydroxynaphthalen-1-yl)-8-fluoro-4-(3-hydroxy-3-methylpiperidin-1-yl)pyrido[4,3-d]pyrimidin-2-yl)oxy)methyl)cyclopropanecarbonitrile (200 mg, 1.0 equiv) and HCl·MeOH (4 M, 1 mL) under N₂ atmosphere. The suspension was degassed and purged with H₂ for 3 times. The mixture was stirred under H₂ (15 Psi) at 25° C. for 3 hours. The reaction mixture was filtered, concentrated and purified with prep-HPLC [Phenomenex luna C18 150×25 mm×10 μm; A: water (10 mM FA), B: ACN, B %: 13%-43% over 10 min] to afford the title compound (14.3 mg, 6.9% yield) as a light yellow solid (0.9 formic acid salt); ¹H NMR (400 MHZ, DMSO-d₆) δ=9.23 (s, 1H), 8.38 (s, 1H), 7.77-7.73 (m, 1H), 7.40-7.25 (m, 2H), 7.10-6.97 (m, 1H), 4.43-4.00 (m, 4H), 3.69-3.43 (m, 1H), 3.41-3.25 (m, 1H), 2.82 (d, J=1.6 Hz, 1H), 2.53-2.51 (m, 2H), 2.44-2.26 (m, 3H), 2.25-1.92 (m, 2H), 1.74-1.61 (m, 3H), 1.17 (d, J=9.6 Hz, 3H), 0.81-0.53 (m, 7H); LCMS (ESI, M+1): m/z=550.2.

### Example 537

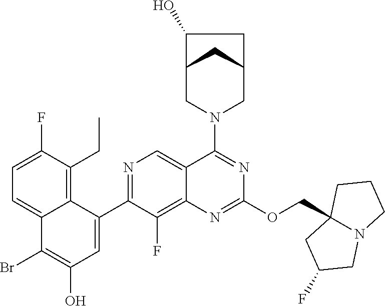 [SMILES: CCc1c(F)ccc2c(Br)c(O)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(4-bromo-8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

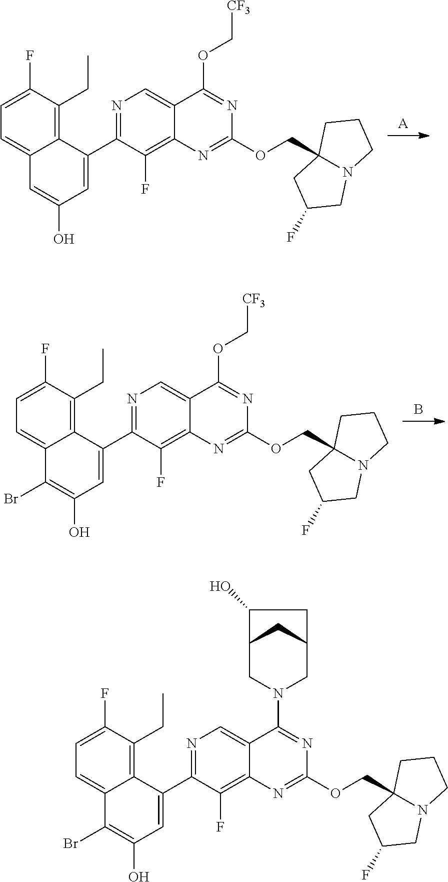

Step A. 1-bromo-5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (340 mg, 1.0 equiv) in DMF (5.0 mL) was added a solution of NBS (112 mg, 1.1 equiv) in DMF (1.5 mL). The mixture was stirred at 20° C. for 2 hours. The reaction mixture was diluted with NazSO₃ (25 mL) and extracted with EtOAc (3×25 mL). The combined organic layers were washed with NaHCO₃ (2×20 mL) and brine (20 mL). The organic was dried over Na₂SO₄, concentrated and purified with prep-HPLC [Phenomenex Synergi Polar-RP 100×25 mm×4 μm; A: water (TFA), B: ACN; B %: 44%-64% over 7 min] to afford the title compound (140 mg, 34% yield) as a yellow solid; ¹H NMR (400 MHZ, DMSO-d₆) δ=10.85 (br s, 1H), 9.22 (s, 1H), 8.21 (dd, J=6.0, 9.6 Hz, 1H), 7.56 (t, J=9.6 Hz, 1H), 7.23 (s, 1H), 5.47-5.17 (m, 3H), 4.34-4.14 (m, 2H), 3.18-3.05 (m, 2H), 3.03 (s, 1H), 2.91-2.78 (m, 1H), 2.39-2.31 (m, 1H), 2.17-2.01 (m, 4H), 1.88-1.77 (m, 3H), 0.71 (br t, J=7.2 Hz, 3H); LCMS (ESI, M+3): m/z=673.0.

Step B. (1R,5R,6R)-3-(7-(4-bromo-8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 1-bromo-5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50 mg, 1.0 equiv) and (1S,5S,6R)-3-azabicyclo[3.2.1]octan-6-ol (18.3 mg, 1.5 equiv, HCl) in DMF (0.15 mL) was added DIPEA (48.1 mg, 5.0 equiv) and 4 Å molecular sieves (20 mg). Then the mixture was stirred at 60° C. for 12 hours. The mixture was filtered, concentrated and purified by prep-HPLC [Phenomenex luna C18 150×25 mm×10 μm; A: water (FA), B: ACN; B %: 18%-48% over 10 min] to afford the title compound (22 mg, 39% yield, FA) as a yellow solid (0.19 formic acid salt); ¹H NMR (400 MHz, DMSO-d₆) δ=10.83 (br s, 1H), 9.39-9.24 (m, 1H), 8.20 (dd, J=6.0, 9.6 Hz, 1H), 8.16 (s, 1H), 7.55 (dt, J=2.8, 9.6 Hz, 1H), 7.23 (d, J=16.0 Hz, 1H), 5.39-5.18 (m, 1H), 4.93-4.66 (m, 2H), 4.64-4.50 (m, 1H), 4.20-4.11 (m, 2H), 4.08-3.95 (m, 1H), 3.80-3.68 (m, 1H), 3.37 (br d, J=13.2 Hz, 1H), 3.10 (br d, J=8.8 Hz, 2H), 3.03 (br s, 1H), 2.84 (br d, J=6.4 Hz, 1H), 2.43-2.31 (m, 2H), 2.20-1.99 (m, 6H), 1.89-1.74 (m, 4H), 1.67 (br s, 1H), 1.30-1.21 (m, 1H), 0.73 (br t, J=7.2 Hz, 3H); LCMS (ESI, M+3): m/z=698.0, 700.0.

### Example 538

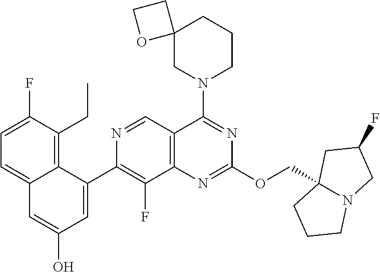 [SMILES: CCc1c(F)ccc2cc(O)cc(-c3ncc4c(N5CCCC6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(1-oxa-6-azaspiro[3.5]nonan-6-yl)pyrido[4,3-d]pyrimidin-7-yl)naphthalen-2-ol

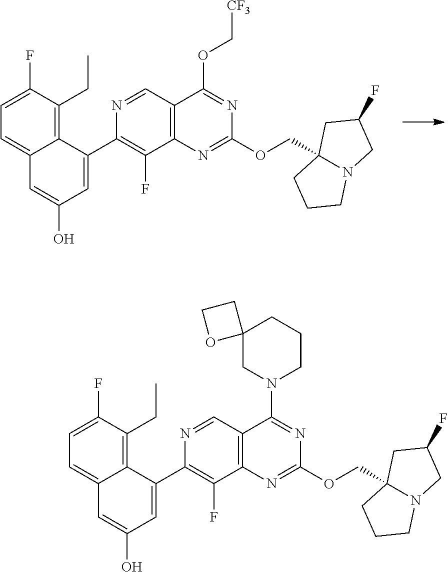 [SMILES: CCc1c(F)ccc2cc(O)cc(-c3ncc4c(OCC(F)(F)F)nc(OC[C@@]56CCCN5C[C@H](F)C6)nc4c3F)c12 ; CCc1c(F)ccc2cc(O)cc(-c3ncc4c(N5CCCC6(CCO6)C5)nc(OC[C@@]56CCCN5C[C@H](F)C6)nc4c3F)c12]

A mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (350 mg, 1.00 equiv), 1-oxa-8-azaspiro[3.5]nonane oxalate (305 mg, 1.5 equiv), K₃PO₄ (1.25 g, 10 equiv) and 4 Å molecular sieves (100 mg) in DMF (2 mL) and ACN (2 mL) was stirred at 60° C. for 2 hours under N₂ atmosphere. The mixture was filtered and purified by prep-HPLC [column: Waters Xbridge 150×25 mm×5 um; mobile phase: [water (ammonia hydroxide)-ACN]; B %: 41%-71%, 9 min] to afford the title compound (140 mg, 36% yield) as a white solid. ¹H NMR (400 MHZ, METHANOL-d4) δ=9.29-9.21 (m, 1H), 7.67 (dd, J=6.0, 9.2 Hz, 1H), 7.30 (d, J=2.8 Hz, 1H), 7.24 (t, J=9.6 Hz, 1H), 7.08 (d, J=2.8 Hz, 1H), 5.44-5.16 (m, 1H), 4.68-4.52 (m, 3H), 4.49-4.36 (m, 1H), 4.35-4.29 (m, 1H), 4.28-4.20 (m, 1H), 3.82 (ddd, J=2.4, 13.6, 19.4 Hz, 1H), 3.54-3.37 (m, 1H), 3.25-3.13 (m, 3H), 3.00 (dt, J=6.0, 9.2 Hz, 1H), 2.54-2.44 (m, 3H), 2.37-2.12 (m, 5H), 2.02-1.76 (m, 6H), 0.87-0.69 (m, 3H). LCMS (ESI, M+1): m/z=620.1

### Example 539

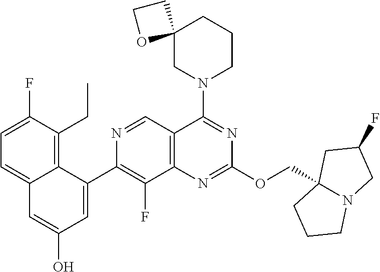 [SMILES: CCc1c(F)ccc2cc(O)cc(-c3ncc4c(N5CCC[C@@]6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-((R)-1-oxa-6-azaspiro[3.5]nonan-6-yl)pyrido[4,3-d]pyrimidin-7-yl)naphthalen-2-ol

### Example 540

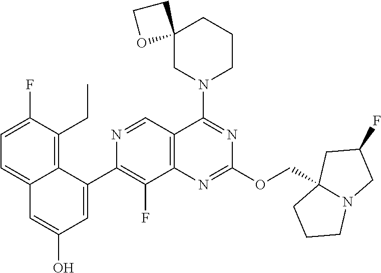 [SMILES: CCc1c(F)ccc2cc(O)cc(-c3ncc4c(N5CCC[C@]6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-((S)-1-oxa-6-azaspiro[3.5]nonan-6-yl)pyrido[4,3-d]pyrimidin-7-yl)naphthalen-2-ol

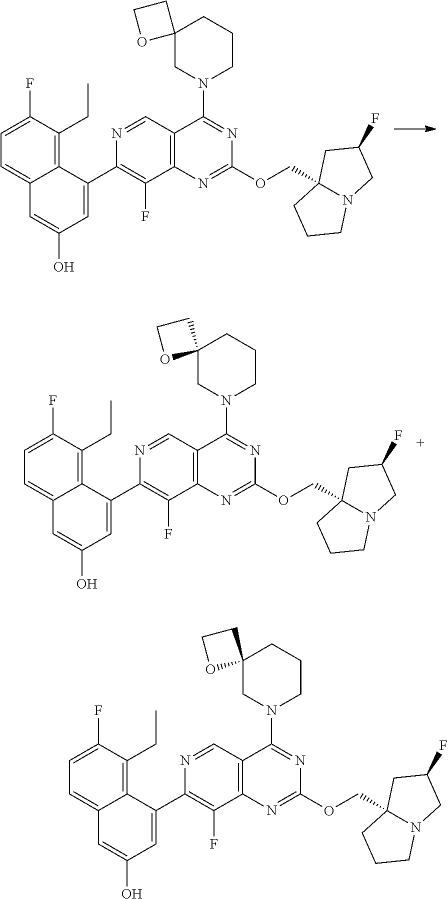 [SMILES: CCc1c(F)ccc2cc(O)cc(-c3ncc4c(N5CCCC6(CCO6)C5)nc(OC[C@@]56CCCN5C[C@H](F)C6)nc4c3F)c12 ; CCc1c(F)ccc2cc(O)cc(-c3ncc4c(N5CCC[C@@]6(CCO6)C5)nc(OC[C@@]56CCCN5C[C@H](F)C6)nc4c3F)c12 ; CCc1c(F)ccc2cc(O)cc(-c3ncc4c(N5CCC[C@]6(CCO6)C5)nc(OC[C@@]56CCCN5C[C@H](F)C6)nc4c3F)c12]

5-ethyl-6-fluoro-4-(8-fluoro-2-(((2R,7aS)-2-fluorohexahydro-1H-pyrrolizin-7a-yl)methoxy)-4-(1-oxa-6-azaspiro[3.5]nonan-6-yl)pyrido[4,3-d]pyrimidin-7-yl)naphthalen-2-ol was separated by SFC [condition: column: REGIS (S,S) WHELK-01 (250 mm×25 mm, 10 um); mobile phase: [0.1% NH₃H₂O ETOH]; B %: 40%-40%, 5.5 min] to afford

Example 539 (51.3 mg, 36% yield) as a yellow solid. ¹H NMR (400 MHZ, METHANOL-d4) δ=9.26 (d, J=4.4 Hz, 1H), 7.68 (dd, J=6.0, 8.8 Hz, 1H), 7.30 (d, J=2.8 Hz, 1H), 7.25 (t, J=9.6 Hz, 1H), 7.08 (d, J=2.8 Hz, 1H), 5.40-5.20 (m, 1H), 4.70-4.53 (m, 3H), 4.50-4.37 (m, 1H), 4.37-4.29 (m, 1H), 4.29-4.20 (m, 1H), 3.84 (dd, J=13.6, 17.6 Hz, 1H), 3.57-3.37 (m, 1H), 3.25-3.12 (m, 3H), 3.05-2.96 (m, 1H), 2.56-2.43 (m, 3H), 2.38-2.12 (m, 5H), 2.03-1.79 (m, 6H), 0.81 (q, J=7.6 Hz, 3H), LCMS (ESI, M+1): m/z=620.3.

Example 540 (49.5 mg, 34.7% yield,) as a yellow solid. ¹H NMR (400 MHZ, METHANOL-d4) δ=9.26 (d, J=4.0 Hz, 1H), 7.67 (dd, J=6.0, 9.2 Hz, 1H), 7.29 (d, J=2.4 Hz, 1H), 7.24 (t, J=9.6 Hz, 1H), 7.07 (d, J=2.4 Hz, 1H), 5.39-5.22 (m, 1H), 4.69-4.52 (m, 3H), 4.50-4.39 (m, 1H), 4.36-4.31 (m, 1H), 4.29-4.23 (m, 1H), 3.84 (dd, J=13.6, 18.0 Hz, 1H), 3.52-3.39 (m, 1H), 3.26-3.18 (m, 3H), 3.05-2.96 (m, 1H), 2.54-2.44 (m, 3H), 2.34-2.13 (m, 5H), 2.03-1.82 (m, 6H), 0.81 (dt, J=3.6, 7.6 Hz, 3H), LCMS (ESI, M+1): m/z=620.3.

### Example 541

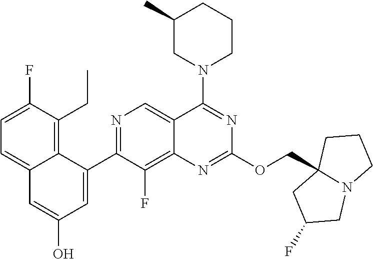 [SMILES: CCc1c(F)ccc2cc(O)cc(-c3ncc4c(N5CCC[C@H](C)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-((S)-3-methylpiperidin-1-yl)pyrido[4,3-d]pyrimidin-7-yl)naphthalen-2-ol

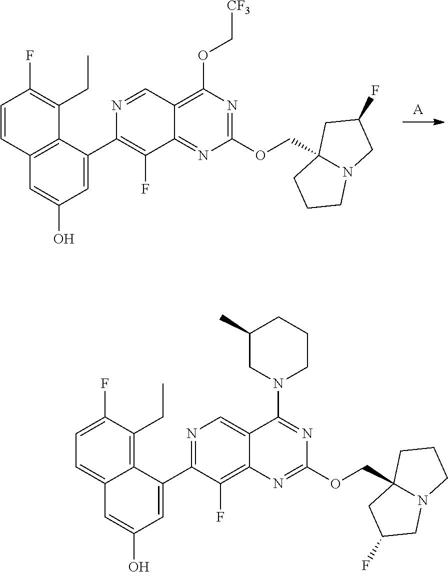

To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (100 mg, 1 equiv) and(S)-3-methylpiperidine (25.1 mg, 1.5 equiv) in DMF (0.8 mL) was added DIPEA (109 mg, 5 equiv) and 4 Å molecular sieves (20 mg). The mixture was stirred at 40° C. for 12 hours. The mixture was diluted with H₂O (10 mL) and extracted with ethyl acetate (4×5 mL). The combined organic layers were dried over anhydrous Na₂SO₄, concentrated and purified with prep-HPLC [column: Phenomenex Synergi C18 150×25 mm×10 um; mobile phase: water (FA)-ACN; B %: 20%-50%, 10 minutes] to afford the title compound (44.5 mg, 41% yield, 0.2FA) as a white solid (1 formic acid salt). ¹H NMR (400 MHZ, METHANOL-d₄) δ=9.02 (s, 1H), 7.68 (dd, J=6.0, 9.2 Hz, 1H), 7.35-7.19 (m, 2H), 7.06 (s, 1H), 5.48-5.24 (m, 1H), 4.68-4.49 (m, 2H), 4.44-4.27 (m, 2H), 3.56-3.32 (m, 4H), 3.18-3.04 (m, 2H), 2.49-1.75 (m, 12H), 1.40 (br d, J=11.2 Hz, 1H), 1.03 (br d, J=6.4 Hz, 3H), 0.80 (t, J=7.2 Hz, 3H); LCMS (ESI, M+1): m/z=592.3

### Example 542

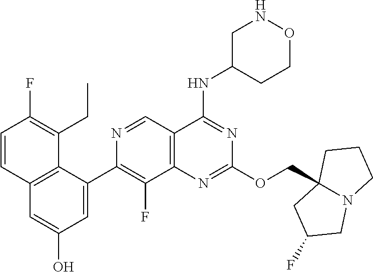 [SMILES: CCc1c(F)ccc2cc(O)cc(-c3ncc4c(NC5CCONC5)nc(OC[C@@]56CCCN5C[C@H](F)C6)nc4c3F)c12]

### 4-(4-((1,2-oxazinan-4-yl)amino)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

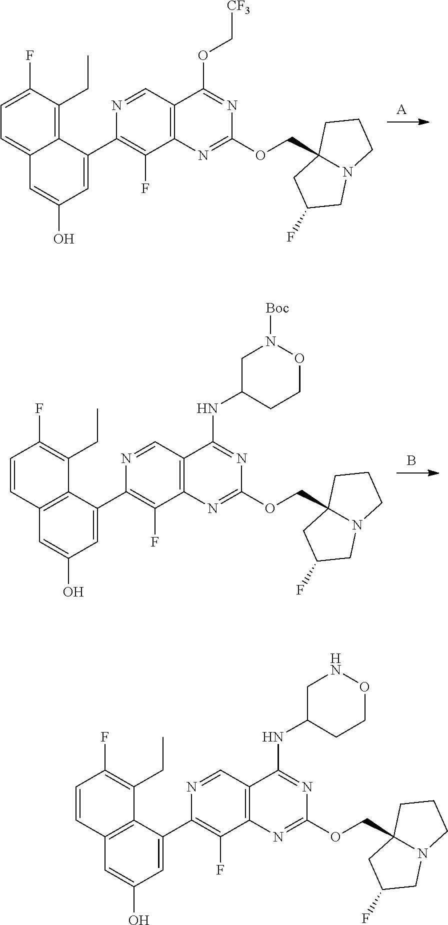

Step A tert-butyl 4-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)-1,2-oxazinane-2-carboxylate: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30.0 mg, 1.0 equiv) in dimethyl formamide (1.0 mL) were added K₃PO₄ (107 mg, 10.0 equiv) and tert-butyl 4-aminooxazinane-2-carboxylate (20.5 mg, 2.0 equiv). The mixture was stirred at 60° C. for 2 hours. The reaction mixture was filtered and concentrated under reduced pressure to afford the title compound (40.0 mg, crude), LCMS (ESI, M+1): m/z=695.3.

Step B. 4-(4-((1,2-oxazinan-4-yl)amino)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol: To a solution of tert-butyl tert-butyl 4-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)-1,2-oxazinane-2-carboxylate (30.0 mg, 1.0 equiv) in DCM (1.0 mL) was added TFA (0.2 mL). The mixture was stirred at 25° C. for 1 hours. The reaction mixture was concentrated to give a residue which was purified by prep-HPLC [column: Waters Xbridge 150×25 mm×5 μm; mobile phase: [water (NH4HCO3)-ACN]; B %: 35%-65%, 10 minutes] and lyophilized to afford the title compound (8.04 mg, 30% yield, 97% purity) as a white solid; 1H NMR (400 MHZ, DMSO-d6) δ=9.96 (s, 1H), 9.43 (s, 1H), 7.77 (dd, J=6.0, 9.2 Hz, 1H), 7.42-7.31 (m, 2H), 7.2-6.94 (m, 2H), 5.70-5.23 (m, 1H), 4.55-4.41 (m, 2H), 4.11-3.98 (m, 1H), 3.80 (br t, J=10.8 Hz, 1H), 3.26-3.11 (m, 3H), 3.06-2.96 (m, 1H), 2.96-2.84 (m, 1H), 2.81-2.59 (m, 1H), 2.41-2.26 (m, 4H), 2.15-1.84 (m, 7H), 0.71 (t, J=7.2 Hz, 3H), LCMS (ESI, M+1): m/z=595.4.

### Example 543

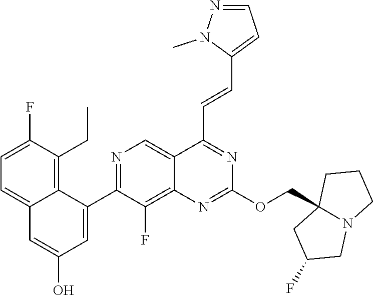 [SMILES: CCc1c(F)ccc2cc(O)cc(-c3ncc4c(/C=C/c5ccnn5C)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((E)-2-(1-methyl-1H-pyrazol-5-yl) vinyl)pyrido[4,3-d]pyrimidin-7-yl)naphthalen-2-ol

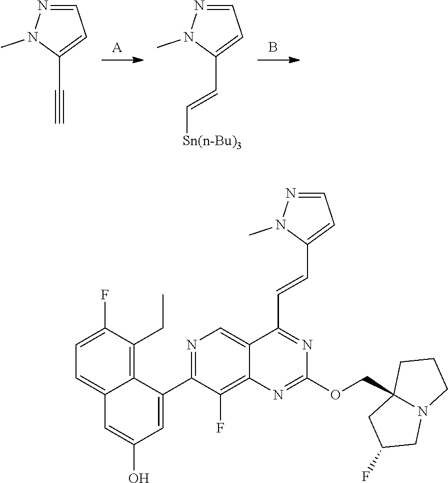

Step A. (E)-1-methyl-5-(2-(tributylstannyl) vinyl)-1H-pyrazole: To a solution of 5-ethynyl-1-methyl-pyrazole (477 mg, 1.0 equiv) and Bu₃SnH (1.7 g, 1.3 equiv) in toluene (7 mL) was added AIBN (36.9 mg, 0.05 equiv). The mixture was stirred at 80° C. for 2 hours. The reaction mixture was concentrated under reduced pressure to give a residue. The residue was purified by flash silica gel chromatography (0˜35% Ethyl acetate/Petroleum ether) to afford the title compound (150 mg, 5.4% yield) as a yellow oil. ¹H NMR (400 MHZ, CHLOROFORM-d) δ=7.44 (d, J=1.6 Hz, 1H), 6.49-6.45 (m, 1H), 6.37 (d, J=1.6 Hz, 1H), 5.71 (d, J=2.8 Hz, 1H), 3.79 (s, 3H), 1.56-1.52 (m, 6H), 1.49-1.43 (m, 6H), 1.34-1.31 (m, 6H), 0.92-0.90 (m, 9H).

Step B. 5-ethyl-6-fluoro-4-(8-fluoro-2-(((2R,7aS)-2-fluorotetrahydro-1H-pyrrolizin-7a(5H)-yl)methoxy)-4-((E)-2-(1-methyl-1H-pyrazol-5-yl) vinyl)pyrido[4,3-d]pyrimidin-7-yl)naphthalen-2-ol: A mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (35 mg, 1.0 equiv), (E)-1-methyl-5-(2-(tributylstannyl) vinyl)-1H-pyrazole (33.4 mg, 1.3 equiv), thiophene-2-carbonyloxycopper (18.5 mg, 1.5 equiv), tris(2-furyl)phosphane (1.5 mg, 0.1 equiv) and Pd₂(dba)₃ (17.8 mg, 0.3 equiv) in THF (1 mL) was stirred at 60° C. for 12 hours under N₂ atmosphere. The mixture was diluted with water (5 mL) and extracted with EtOAc (5 mL×3). The combined organic layers were washed with brine (10 mL), dried over Na₂SO₄, filtered and purified by prep-TLC (SiO₂, DCM/MeOH 10:1), followed by prep-HPLC [C18, 0.1% formic acid condition] and lyophilized to afford the title compound (4 mg, 10.0% yield) as a yellow solid. ¹H NMR (400 MHZ, CHLOROFORM-d) δ=9.31 (d, J=6.0 Hz, 1H), 8.26-8.12 (m, 1H), 7.63 (dd, J=11.2, 15.2 Hz, 1H), 7.57-7.48 (m, 2H), 7.23-7.16 (m, 2H), 7.10-6.86 (m, 1H), 6.78 (dd, J=1.6, 14.0 Hz, 1H), 5.48-5.24 (m, 1H), 4.67-4.46 (m, 2H), 4.05 (d, J=6.0 Hz, 3H), 3.64-3.48 (m, 2H), 3.44-3.25 (m, 2H), 3.14-3.07 (m, 1H), 2.50-2.17 (m, 6H), 0.85-0.67 (m, 4H); LCMS (ESI, M+1): m/z=601.2.

### Example 544

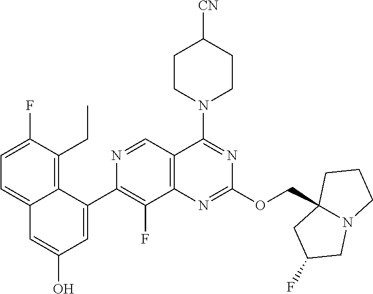 [SMILES: CCc1c(F)ccc2cc(O)cc(-c3ncc4c(N5CCC(C#N)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)piperidine-4-carbonitrile

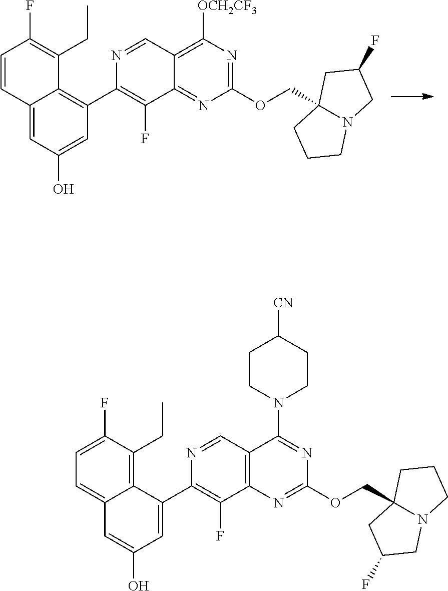

To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (60.0 mg, 1.0 equiv) and piperidine-4-carbonitrile;hydrochloride (55.6 mg, 3.0 equiv, HCl) in DMF (2 mL) was added potassium phosphate (214 mg, 10 equiv) and 4 Å molecular sieves (10 mg). The mixture was stirred at 60° C. for 2 hours. The mixture was filtered and purified with prep-HPLC [column: Waters Xbridge 150×25 mm×5 μm; mobile phase: [water (ammonia hydroxide v/v)-ACN]; B %: 43%-73%, 9 minutes] and lyophilized to afford the title compound (16.0 mg, 25% yield,) as a yellow solid; ¹H NMR (400 MHz, CD₃OD) δ=9.04 (s, 1H), 7.65 (dd, J=6.0, 8.8 Hz, 1H), 7.27 (d, J=2.4 Hz, 1H), 7.23 (t, J=9.6 Hz, 1H), 7.04 (d, J=2.4 Hz, 1H), 5.40-5.21 (m, 1H), 4.34-4.24 (m, 4H), 3.96-3.87 (m, 2H), 3.28-3.17 (m, 4H), 3.04-2.98 (m, 1H), 2.93 (s, 1H), 2.53-2.43 (m, 1H), 2.29-2.04 (m, 8H), 2.02-1.95 (m, 2H), 1.92-1.84 (m, 1H), 0.79 (t, J=6.8 Hz, 3H); LCMS (ESI, M+1): m/z=603.2.

### Example 545

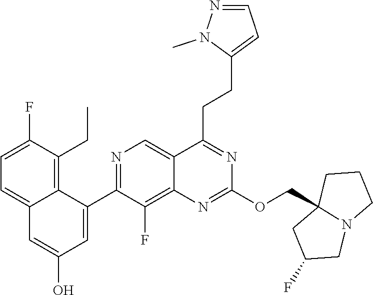 [SMILES: CCc1c(F)ccc2cc(O)cc(-c3ncc4c(CCc5ccnn5C)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(2-(1-methyl-1H-pyrazol-5-yl)ethyl)pyrido[4,3-d]pyrimidin-7-yl)naphthalen-2-ol

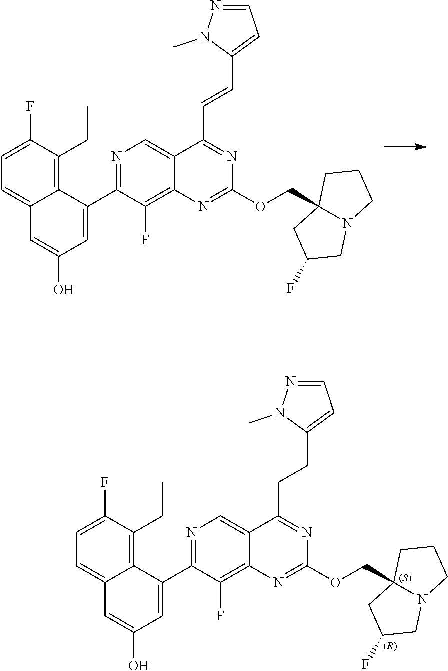

To a solution of 5-ethyl-6-fluoro-4-(8-fluoro-2-(((2R,7aS)-2-fluorotetrahydro-1H-pyrrolizin-7a(5H)-yl)methoxy)-4-((E)-2-(1-methyl-1H-pyrazol-5-yl) vinyl)pyrido[4,3-d]pyrimidin-7-yl)naphthalen-2-ol (10 mg, 1 equiv) in THF (0.5 mL) was added Pd/C (5 mg, 10% purity). The mixture was degassed and purged with H₂ for 3 times, and then the mixture was stirred at 20° C. for 12 hours under H₂ atmosphere (15 Psi). The reaction mixture was filtered and purified by prep-HPLC [C18, 0.1% NH₄HCO₃ condition] and lyophilized to afford the title compound (1.4 mg, 12.8% yield) as a white solid. 1H NMR (400 MHZ, CHLOROFORM-d) δ=7.14 (d, J=8.0 Hz, 1H), 6.48-6.34 (m, 2H), 4.66 (s, 2H), 3.78 (d, J=8.4 Hz, 6H), 3.58-3.46 (m, 2H), 2.86-2.73 (m, 2H), 2.65-2.50 (m, 2H); LCMS (ESI, M+1): m/z=603.1.

### Example 546

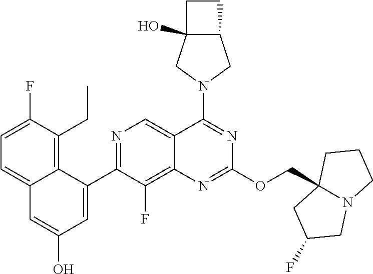 [SMILES: CCc1c(F)ccc2cc(O)cc(-c3ncc4c(N5C[C@H]6CC[C@@]6(O)C5)nc(OC[C@@]56CCCN5C[C@H](F)C6)nc4c3F)c12]

(1S,5R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.0]heptan-1-ol The title compound was synthesized according to the procedure described for example 538 as a yellow solid (0.38 formic acid salt). ¹H NMR (400 MHZ, CD₃OD) δ=9.29 (d, J=2.8 Hz, 1H), 7.68 (dd, J=6.0, 9.2 Hz, 1H), 7.31 (d, J=2.4 Hz, 1H), 7.25 (t, J=9.6 Hz, 1H), 7.06 (dd, J=2.4, 4.8 Hz, 1H), 5.50-5.32 (m, 1H), 4.57-4.46 (m, 2H), 4.45-4.37 (m, 1H), 4.36-4.24 (m, 1H), 4.23-4.12 (m, 2H), 3.63-3.41 (m, 3H), 3.22-3.14 (m, 1H), 3.05-2.96 (m, 1H), 2.52-2.39 (m, 2H), 2.38-2.30 (m, 2H), 2.29-2.21 (m, 2H), 2.19-2.07 (m, 4H), 2.06-1.97 (m, 1H), 1.50-1.36 (m, 1H), 0.83-0.77 (m, 3H); LCMS (ESI, M+1): m/z=606.3.

### Example 547

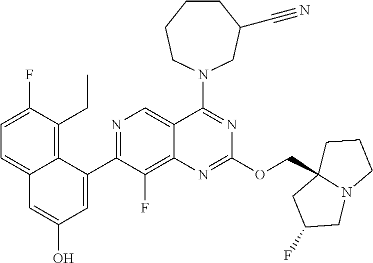 [SMILES: CCc1c(F)ccc2cc(O)cc(-c3ncc4c(N5CCCCC(C#N)C5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)azepane-3-carbonitrile

The title compound was synthesized according to the procedure described for example 538 except for heating of the reaction mixture was carried out at 40° C. for 15 hours to afford the desired compound as a yellow solid. ¹HNMR (400 MHZ, methanol-d4) δ=9.21-9.24 (m, 1H), 8.50 (s, 1H), 7.68 (dd, J=9.2, 6.0 Hz, 1H), 7.31 (d, J=2.4 Hz, 1H), 7.25 (t, J=9.2 Hz, 1H), 7.02-7.08 (m, 1H), 5.28-5.51 (m, 1H), 4.43-4.58 (m, 3H), 4.03-4.35 (m, 3H), 3.39-3.65 (m, 4H), 3.13-3.23 (m, 1H), 2.04-2.59 (m, 11H), 1.94-2.01 (m, 1H), 1.81-1.92 (m, 1H), 1.48-1.65 (m, 1H), 0.75-0.84 ppm (m, 3H); LCMS (ESI, M+1): m/z=617.4.

### Example 548

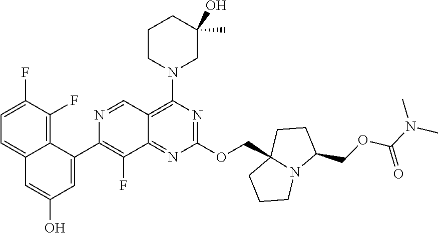 [SMILES: CN(C)C(=O)OC[C@@H]1CC[C@@]2(COc3nc(N4CCC[C@@](C)(O)C4)c4cnc(-c5cc(O)cc6ccc(F)c(F)c56)c(F)c4n3)CCCN12]

### ((3S,7aR)-7a-(((7-(7,8-difluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate

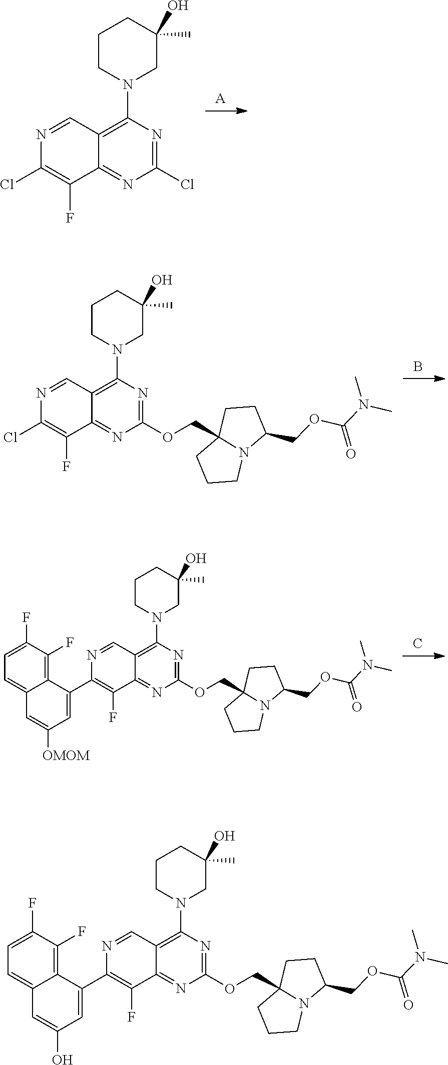

Step A ((3S,7aR)-7a-(((7-chloro-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: To a solution of (R)-1-(2,7-dichloro-8-fluoropyrido[4,3-d]pyrimidin-4-yl)-3-methylpiperidin-3-ol (165 mg, 1.1 equiv) in toluene (5.0 mL) were added ((3S,7aR)-7a-(hydroxymethyl)hexahydro-1H-pyrrolizin-3-yl)methyl dimethylcarbamate (110 mg, 1.0 equiv), t-BuONa (131 mg, 3.0 equiv) and 4 Å molecular sieves (20.0 mg). The mixture was stirred at 0° C. for 1 hour. The reaction mixture was partitioned between ethyl acetate (50 mL) and water (30 mL). The organic phase was separated and concentrated under reduced pressure to give a residue. The residue was purified with prep-TLC (SiO₂, dichloromethane/methyl alcohol 10:1) to afford the title compound (70.0 mg, 29% yield), LCMS (ESI, M+1): m/z=537.1

Step B ((3S,7aR)-7a-(((7-(7,8-difluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: To a solution of ((3S,7aR)-7a-(((7-chloro-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate) (140 mg, 1.0 equiv) in dioxane (2.00 mL) and H₂O (0.50 mL) was added K₃PO₄ (166 mg, 3.0 equiv) CataCXium A Pd G3 (19.0 mg, 0.1 equiv) and 2-(7,8-difluoro-3-(methoxymethoxy)naphthalen-1-yl)-4,4,5,5-tetramethyl-1,3,2-dioxaborolane (100 mg, 1.1 equiv). The mixture was stirred at 90° C. for 2 hours. The reaction mixture was concentrated under reduced pressure to give a residue. The residue was purified by prep-TLC (SiO2, DCM/MeOH 10:1) to afford the title compound (80.0 mg, 42% yield) as a yellow solid. LCMS (ESI, M+1): m/z=725.4

Step C ((3S,7aR)-7a-(((7-(7,8-difluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: To a solution of ((3S,7aR)-7a-(((7-(7,8-difluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate (70.0 mg, 1.0 equiv) in EtOAc (3.00 mL) was added HCl/MeOH (4 M, 3.00 mL). The mixture was stirred at 0° C. for 1 hour. The reaction mixture was concentrated under reduced pressure to give a residue. The residue was purified with prep-HPLC (column: Phenomenex C18 75×30 mm×3 um; mobile phase: [water (FA)-ACN]; B %: 15%-45%, 7 min) to afford the title compound (5.15 mg, 7% yield,) as a white solid (1 formic acid salt); ¹H NMR (400 MHZ, METHANOL-d4) δ=9.22 (d, J=4.4 Hz, 1H), 8.53 (s, 1H), 7.61 (dd, J=4.4, 8.8 Hz, 1H), 7.44-7.35 (m, 1H), 7.33 (s, 1H), 7.24 (dd, J=2.4, 6.4 Hz, 1H), 4.61-4.53 (m, 1H), 4.40-4.26 (m, 3H), 4.16 (td, J=5.2, 10.4 Hz, 1H), 4.03 (ddd, J=4.0, 6.8, 10.8 Hz, 1H), 3.65-3.59 (m, 1H), 3.48-3.39 (m, 1H), 3.25-3.13 (m, 2H), 3.02-2.94 (m, 1H), 2.93-2.84 (m, 6H), 2.28 (td, J=6.4, 12.6 Hz, 1H), 2.21 (br d, J=2.8 Hz, 1H), 2.12-1.90 (m, 5H), 1.89-1.76 (m, 5H), 1.31-1.27 (m, 3H), LCMS (ESI, M+1): m/z=681.4

### Example 549

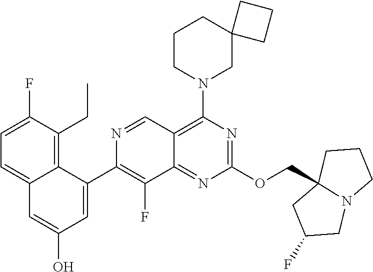 [SMILES: CCc1c(F)ccc2cc(O)cc(-c3ncc4c(N5CCCC6(CCC6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(6-azaspiro[3.5]nonan-6-yl)pyrido[4,3-d]pyrimidin-7-yl)naphthalen-2-ol

The title compound was synthesized according to the procedure described for example 541 to produce the desired compound as a yellow solid (0.21 formic acid salt); ¹H NMR (400 MHZ, methanol-d4) δ=9.08 (s, 1H), 7.68 (dd, J=6.0, 8.8 Hz, 1H), 7.31 (d, J=2.4 Hz, 1H), 7.25 (t, J=9.2 Hz, 1H), 7.08 (d, J=2.4 Hz, 1H), 5.35 (d, J=54.0 Hz, 1H), 4.42-4.32 (m, 2H), 4.17 (br d, J=12.8 Hz, 1H), 4.13-4.05 (m, 1H), 4.00 (br d, J=12.8 Hz, 1H), 3.91-3.81 (m, 1H), 3.36-3.33 (m, 3H), 3.15-3.05 (m, 1H), 2.54-2.26 (m, 3H), 2.24-2.13 (m, 2H), 2.11-2.00 (m, 2H), 1.99-1.89 (m, 3H), 1.88-1.73 (m, 8H), 1.74-1.73 (m, 1H), 0.80 (br t, J=7.2 Hz, 3H); LCMS (ESI, M+1): m/z=618.3.

### Example 550

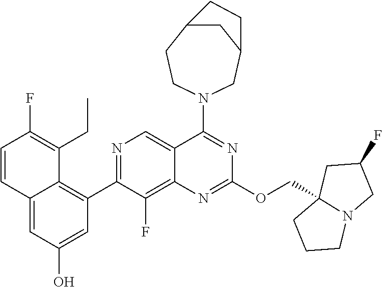 [SMILES: CCc1c(F)ccc2cc(O)cc(-c3ncc4c(N5CCC6CCC(C6)C5)nc(OC[C@@]56CCCN5C[C@H](F)C6)nc4c3F)c12]

### 4-(4-(3-azabicyclo[4.2.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

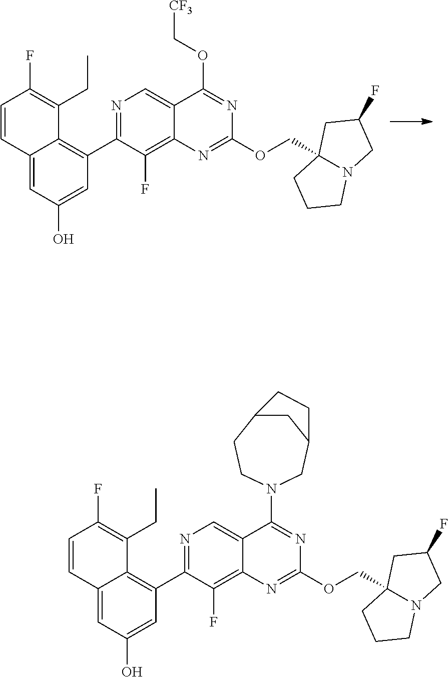 [SMILES: CCc1c(F)ccc2cc(O)cc(-c3ncc4c(OCC(F)(F)F)nc(OC[C@@]56CCCN5C[C@H](F)C6)nc4c3F)c12 ; CCc1c(F)ccc2cc(O)cc(-c3ncc4c(N5CCC6CCC(C6)C5)nc(OC[C@@]56CCCN5C[C@H](F)C6)nc4c3F)c12]

To a solution of 3-azabicyclo[4.2.1]nonane (20.5 mg, 3.0 equiv, HCl) in DMF (0.1 mL) was added K₃PO₄ (44.8 mg, 5.0 equiv). The mixture was stirred at 25° C. for 0.5 hour. Then 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (25.0 mg, 1.0 equiv) and ACN (0.1 mL) was added. The mixture was stirred at 40° C. for 11.5 hours. The resulting mixture was filtered and purified by prep-HPLC [Waters Xbridge 150×25 mm×5 um; A: water (0.1% NH₄HCO₃), B: ACN, B %: 56%-86% over 8 min] and lyophilized to afford the title compound (6.07 mg, 23% yield) as a white solid; ¹H NMR (400 MHZ, DMSO-d₆) δ=10.19-9.66 (m, 1H), 9.11 (s, 1H), 7.77 (dd, J=6.0, 9.2 Hz, 1H), 7.38-7.32 (m, 2H), 7.04 (d, J=2.8 Hz, 1H), 5.38-5.21 (m, 1H), 4.32-3.76 (m, 6H), 3.70-3.62 (m, 1H), 3.09 (br d, J=6.4 Hz, 2H), 3.01 (s, 1H), 2.83 (br d, J=6.0 Hz, 1H), 2.65-2.61 (m, 1H), 2.13 (br d, J=4.8 Hz, 2H), 2.03-1.71 (m, 10H), 1.67-1.54 (m, 2H), 1.46-1.38 (m, 1H), 1.24 (s, 1H), 0.77-0.70 (m, 3H); LCMS (ESI, M+1): m/z=618.3.

### Example 551

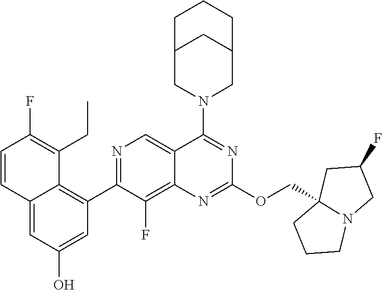 [SMILES: CCc1c(F)ccc2cc(O)cc(-c3ncc4c(N5CC6CCCC(C6)C5)nc(OC[C@@]56CCCN5C[C@H](F)C6)nc4c3F)c12]

### 4-(4-(3-azabicyclo[3.3.1]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 550 to produce the desired compound as a white solid; ¹H NMR (400 MHZ, DMSO-d₆) δ=9.95 (br s, 1H), 9.16 (s, 1H), 7.77 (dd, J=6.0, 9.2 Hz, 1H), 7.39-7.31 (m, 2H), 7.04 (d, J=2.4 Hz, 1H), 5.39-5.21 (m, 1H), 4.74-4.64 (m, 2H), 4.19-4.08 (m, 2H), 3.78-3.65 (m, 4H), 3.12-3.07 (m, 2H), 2.83 (br d, J=6.0 Hz, 2H), 2.07 (br d, J=10.8 Hz, 5H), 1.91-1.72 (m, 8H), 1.67 (br s, 2H), 1.48-1.41 (m, 1H), 0.73 (t, J=7.2 Hz, 3H); LCMS (ESI, M+1): m/z=618.5.

### Example 552

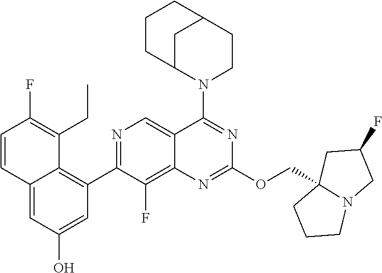 [SMILES: CCc1c(F)ccc2cc(O)cc(-c3ncc4c(N5CCC6CCCC5C6)nc(OC[C@@]56CCCN5C[C@H](F)C6)nc4c3F)c12]

### 4-(4-(2-azabicyclo[3.3.1]nonan-2-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 550 to produce the desired compound as a white solid; ¹H NMR (400 MHZ, DMSO-d₆) δ=10.03-9.86 (m, 1H), 9.16 (d, J=5.2 Hz, 1H), 7.76 (dd, J=6.0, 9.0 Hz, 1H), 7.38-7.32 (m, 2H), 7.03-6.99 (m, 1H), 5.36-5.20 (m, 1H), 5.04 (br d, J=9.9 Hz, 1H), 4.38-4.27 (m, 1H), 4.19-3.93 (m, 4H), 3.13-3.00 (m, 4H), 2.82 (br d, J=6.4 Hz, 1H), 2.33 (br s, 2H), 2.12 (br d, J=3.5 Hz, 4H), 2.02-1.86 (m, 5H), 1.83-1.72 (m, 6H), 0.73 (br t, J=6.8 Hz, 3H); LCMS (ESI, M+1): m/z=618.5.

### Example 553

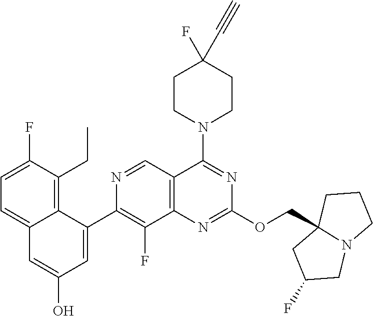 [SMILES: C#CC1(F)CCN(c2nc(OC[C@@]34CCCN3C[C@H](F)C4)nc3c(F)c(-c4cc(O)cc5ccc(F)c(CC)c45)ncc23)CC1]

### 5-ethyl-4-(4-(4-ethynyl-4-fluoropiperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-6-fluoronaphthalen-2-ol

The title compound was synthesized according to the procedure described for example 544 to produce the desired compound as a yellow solid. ¹H NMR (400 MHZ, METHANOL-d4) δ=9.08 (s, 1H), 7.67 (dd, J=5.8, 9.2 Hz, 1H), 7.30 (d, J=2.4 Hz, 1H), 7.24 (t, J=9.6 Hz, 1H), 7.05 (d, J=2.4 Hz, 1H), 5.47-5.14 (m, 1H), 4.37-4.22 (m, 3H), 4.22-4.07 (m, 4H), 3.28-3.16 (m, 3H), 3.01 (dt, J=5.6, 9.2 Hz, 1H), 2.57-2.40 (m, 1H), 2.33-2.09 (m, 8H), 2.03-1.85 (m, 3H), 0.79 (t, J=7.2 Hz, 3H); LCMS (ESI, M+1): m/z=620.3.

### Example 554

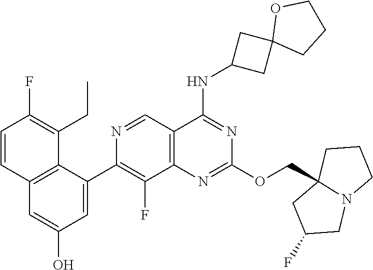 [SMILES: CCc1c(F)ccc2cc(O)cc(-c3ncc4c(NC5CC6(CCCO6)C5)nc(OC[C@@]56CCCN5C[C@H](F)C6)nc4c3F)c12]

### 4-(4-(5-oxaspiro[3.4]octan-2-ylamino)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized according to the procedure (except for no 4A Molecular sieves were added) described for example 538 to produce the desired compound as a white solid; ¹H NMR (400 MHZ, METHANOL-d4) δ=9.31-9.19 (m, 1H), 7.68 (dd, J=6.0, 9.2 Hz, 1H), 7.31 (d, J=2.8 Hz, 1H), 7.25 (t, J=9.6 Hz, 1H), 7.04 (d, J=2.8 Hz, 1H), 5.62-5.31 (m, 1H), 4.62-4.35 (m, 4H), 3.84 (t, J=6.4 Hz, 2H), 3.69-3.49 (m, 3H), 2.70-2.61 (m, 2H), 2.52-2.39 (m, 5H), 2.36-2.27 (m, 1H), 2.23-2.12 (m, 3H), 2.07-1.98 (m, 5H), 0.78 (t, J=7.6 Hz, 3H); LCMS [ESI, M+1]: m/z=620.3.

### Example 555

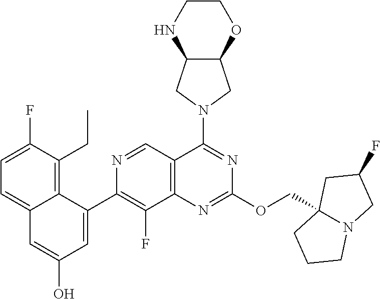 [SMILES: CCc1c(F)ccc2cc(O)cc(-c3ncc4c(N5C[C@@H]6OCCN[C@@H]6C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(hexahydropyrrolo[3,4-b][1,4]oxazin-6 (2H)-yl)pyrido[4,3-d]pyrimidin-7-yl)naphthalen-2-ol

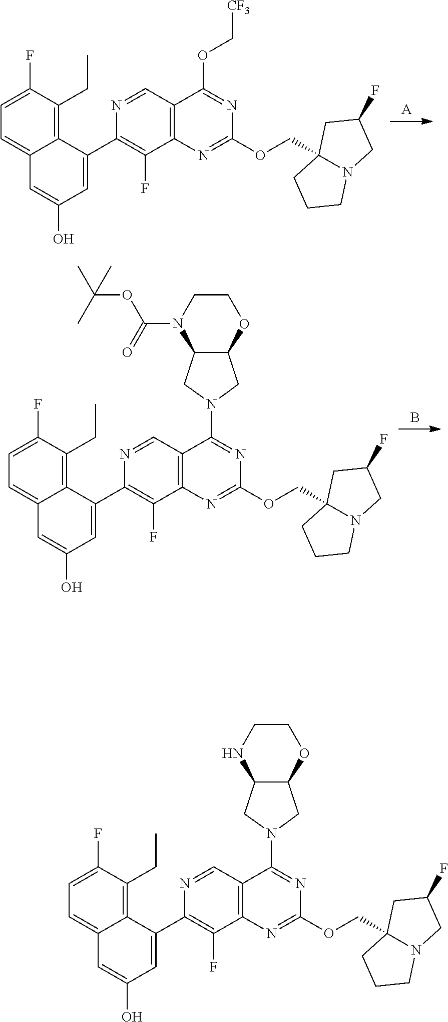

Step A. tert-butyl (4aR,7aS)-6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)hexahydropyrrolo[3,4-b][1,4]oxazine-4(4aH)-carboxylate: A mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (20.0 mg, 1.0 equiv), tert-butyl (4aS,7aR)-3,4a,5,6,7,7a-hexahydro-2H-pyrrolo[3,4-b][1,4]oxazine-4-carboxylate (9.80 mg, 1.1 equiv, HCl), K₃PO₄ (35.8 mg, 5.0 equiv) in DMF (0.5 mL) was degassed and stirred at 40° C. for 2 hours under N₂ atmosphere. The reaction mixture was diluted with water (5 mL) and extracted with EtOAc (3 mL×3). The combined organic layers were washed with brine (8 mL), dried over Na₂SO₄, filtered and purified with prep-HPLC [C18, 0.1% formic acid condition] to afford the tittle compound (20 mg, 82.0% yield) as a white solid; 1H NMR (400 MHZ, CHLOROFORM-d) δ=9.22-9.03 (m, 1H), 7.51 (br d, J=4.4 Hz, 1H), 7.21-7.09 (m, 2H), 7.07-6.95 (m, 1H), 5.41-5.16 (m, 1H), 4.81-4.49 (m, 1H), 4.35-4.02 (m, 6H), 4.01-3.72 (m, 3H), 3.66-3.50 (m, 1H), 3.40-3.13 (m, 4H), 3.05-2.95 (m, 1H), 2.53-2.36 (m, 1H), 2.34-2.20 (m, 2H), 2.19-2.07 (m, 2H), 2.01-1.88 (m, 3H), 0.87-0.74 (m, 3H).

Step B. 5-ethyl-6-fluoro-4-(8-fluoro-2-(((2R,7aS)-2-fluorotetrahydro-1H-pyrrolizin-7a(5H)-yl)methoxy)-4-((4aR,7aS)-hexahydropyrrolo[3,4-b][1.4]oxazin-6 (2H)-yl)pyrido[4,3-d]pyrimidin-7-yl)naphthalen-2-ol: To a solution of tert-butyl (4aR,7aS)-6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)hexahydropyrrolo[3,4-b][1,4]oxazine-4(4aH)-carboxylate (20.0 mg, 1.0 equiv) in DCM (0.3 mL) was added HCl·dioxane (0.3 mL). The mixture was stirred at 20° C. for 1 hour. The reaction mixture was concentrated under reduced pressure to afford the title compound (17 mg, HCl salt) as a yellow solid. ¹H NMR (400 MHZ, methanol-d₄) δ=9.54-9.34 (m, 1H), 7.74 (dd, J=6.0, 9.2 Hz, 1H), 7.41 (d, J=2.4 Hz, 1H), 7.32 (t, J=9.2 Hz, 1H), 7.19 (br d, J=2.8 Hz, 1H), 5.72-5.48 (m, 1H), 5.05-4.91 (m, 2H), 4.74-4.50 (m, 3H), 4.48-4.24 (m, 3H), 4.20-4.02 (m, 2H), 4.02-3.84 (m, 4H), 3.71-3.62 (m, 1H), 3.60 (s, 1H), 3.49 (dt, J=6.0, 10.4 Hz, 1H), 2.83-2.62 (m, 2H), 2.58-2.44 (m, 2H), 2.42-2.33 (m, 2H), 2.29-2.15 (m, 2H), 0.91-0.82 (m, 3H); ¹⁹F NMR (376 MHz, methanol-d₄) δ=−120, −136, −174; LCMS (ESI, M+1): m/z=621.4.

### Example 556

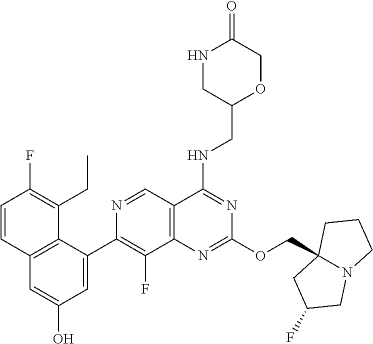 [SMILES: CCc1c(F)ccc2cc(O)cc(-c3ncc4c(NCC5CNC(=O)CO5)nc(OC[C@@]56CCCN5C[C@H](F)C6)nc4c3F)c12]

### 6-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl) morpholin-3-one

The title compound was synthesized according to the procedure described for example 538 to produce the desired compound as a white solid; ¹H NMR (400 MHZ, METHANOL-d4) δ=0.79 (t, J=7.2 Hz, 3H) 1.96-2.07 (m, 1H) 2.09-2.20 (m, 3H) 2.25 (br d, J=8.8 Hz, 1H) 2.30-2.54 (m, 3H) 3.16-3.25 (m, 1H) 3.33-3.41 (m, 1H) 3.43-3.54 (m, 4H) 3.78-3.99 (m, 2H) 4.13-4.29 (m, 3H) 4.40-4.53 (m, 2H) 5.32-5.52 (m, 1H) 7.04 (d, J=2.4 Hz, 1H) 7.25 (t, J=9.2 Hz, 1H) 7.31 (d, J=2.8 Hz, 1H) 7.68 (dd, J=9.2, 5.6 Hz, 1H) 8.49 (br s, 1H) 9.22 (s, 1H); LCMS (ESI, M+1): m/z=623.2 The title compound was synthesized according to the procedure described for example 538 to produce the desired compound as a white solid (0.34 formic acid salt); ¹H NMR (400 MHZ, METHANOL-d4) δ=0.79 (t, J=7.2 Hz, 3H) 1.96-2.07 (m, 1H) 2.09-2.20 (m, 3H) 2.25 (br d, J=8.8 Hz, 1H) 2.30-2.54 (m, 3H) 3.16-3.25 (m, 1H) 3.33-3.41 (m, 1H) 3.43-3.54 (m, 4H) 3.78-3.99 (m, 2H) 4.13-4.29 (m, 3H) 4.40-4.53 (m, 2H) 5.32-5.52 (m, 1H) 7.04 (d, J=2.4 Hz, 1H) 7.25 (t, J=9.2 Hz, 1H) 7.31 (d, J=2.8 Hz, 1H) 7.68 (dd, J=9.2, 5.6 Hz, 1H) 8.49 (br s, 1H) 9.22 (s, 1H).

### Example 557

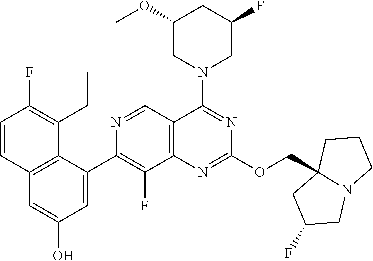 [SMILES: CCc1c(F)ccc2cc(O)cc(-c3ncc4c(N5C[C@H](F)C[C@@H](OC)C5)nc(OC[C@@]56CCCN5C[C@H](F)C6)nc4c3F)c12]

### 5-ethyl-6-fluoro-4-(8-fluoro-4-((3R,5R)-3-fluoro-5-methoxypiperidin-1-yl)-2-(((2R,7aS)-2-fluorohexahydro-1H-pyrrolizin-7a-yl)methoxy)pyrido[4,3-d]pyrimidin-7-yl)naphthalen-2-ol

The title compound was synthesized according to the procedure (except for no 4A Molecular sieves were added) described for example 538 to produce the desired compound as a white solid; ¹H NMR (400 MHZ, METHANOL-d4) δ=9.22 (d, J=5.6 Hz, 1H), 7.68 (dd, J=5.6, 9.2 Hz, 1H), 7.31 (d, J=2.8 Hz, 1H), 7.25 (t, J=9.6 Hz, 1H), 7.07 (dd, J=2.8, 10.8 Hz, 1H), 5.55-5.39 (m, 1H), 5.15-4.99 (m, 1H), 4.61-4.43 (m, 3H), 4.34-4.15 (m, 2H), 3.90-3.65 (m, 3H), 3.65-3.54 (m, 2H), 3.42 (s, 1H), 3.37 (s, 2H), 2.63-2.39 (m, 3H), 2.37-2.26 (m, 2H), 2.25-1.95 (m, 6H), 0.78 (t, J=7.6 Hz, 3H); LCMS (ESI, M+1): m/z=626.4

### Example 558

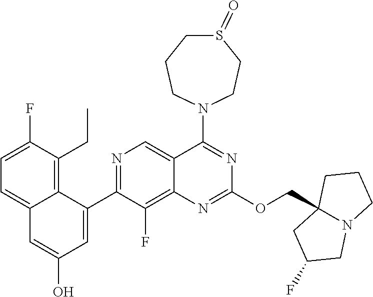 [SMILES: CCc1c(F)ccc2cc(O)cc(-c3ncc4c(N5CCCS(=O)CC5)nc(OC[C@@]56CCCN5C[C@H](F)C6)nc4c3F)c12]

### 4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1,4-thiazepane 1-oxide

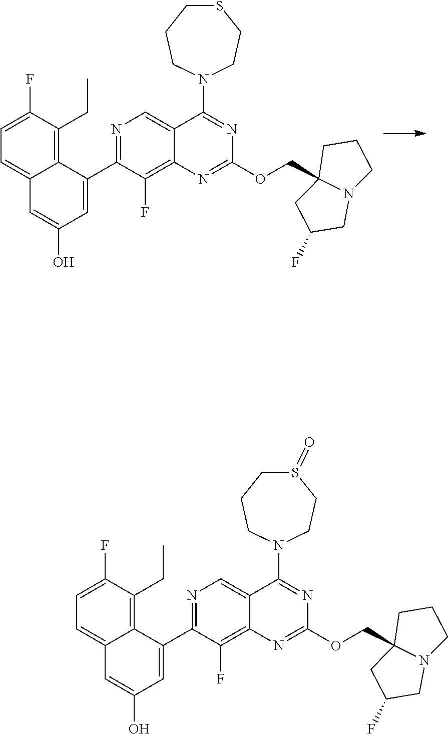 [SMILES: CCc1c(F)ccc2cc(O)cc(-c3ncc4c(N5CCCSCC5)nc(OC[C@@]56CCCN5C[C@H](F)C6)nc4c3F)c12 ; CCc1c(F)ccc2cc(O)cc(-c3ncc4c(N5CCCS(=O)CC5)nc(OC[C@@]56CCCN5C[C@H](F)C6)nc4c3F)c12]

To a solution of NaIO₄ (33.2 mg, 1.1 equiv) in water (1 mL) was added a solution of 5-ethyl-6-fluoro-4-(8-fluoro-2-(((2R,7aS)-2-fluorohexahydro-1H-pyrrolizin-7a-yl)methoxy)-4-(1,4-thiazepan-4-yl)pyrido[4,3-d]pyrimidin-7-yl)naphthalen-2-ol (90.0 mg, 1.0 equiv) in MeOH (0.8 mL) and dioxane (0.6 mL) at 0° C. and the reaction was stirred at 15° C. for 12 hours. The mixture was diluted with water (10 mL) and extracted with ethyl acetate (2×10 mL). The organic layer was washed with brine (10 mL) and dried over Na₂SO₄. The solvent was concentrated to and purified by prep-HPLC [column: Waters Xbridge 150×25 mm×5 μm; A: water (10 mM NH₄HCO₃), B: ACN, B %: 33%-63% over 8 minutes] to afford the title compound (45.1 mg, 49% yield) as a yellow oil. ¹H NMR (400 MHZ, METHANOL-d₄) δ=9.21 (s, 1H), 7.68 (dd, J=5.6, 9.2 Hz, 1H), 7.40-7.17 (m, 2H), 7.04 (dd, J=2.4, 17.2 Hz, 1H), 5.43-5.21 (m, 1H), 4.51 (br d, J=1.8 Hz, 1H), 4.43-4.04 (m, 5H), 3.40 (br s, 2H), 3.35 (br s, 1H), 3.29-3.09 (m, 3H), 3.08-2.77 (m, 3H), 2.59-2.41 (m, 1H), 2.39-2.09 (m, 5H), 2.08-1.78 (m, 3H), 0.87-0.72 (m, 3H); LCMS (ESI, M+1): m/z=626.3.

### Example 559

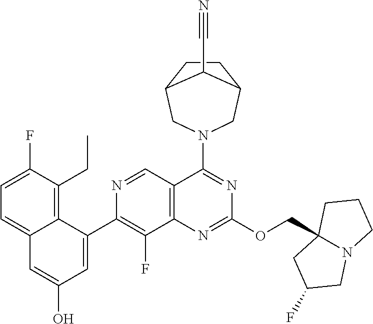 [SMILES: CCc1c(F)ccc2cc(O)cc(-c3ncc4c(N5CC6CCC(C5)C6C#N)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carbonitrile

The title compound was synthesized according to the procedure described for example 544 to produce the desired compound as a white solid (0.31 formic acid salt); ¹H NMR (400 MHZ, METHANOL-d4) δ=9.17-9.03 (m, 1H), 8.51 (br s, 1H), 7.68 (dd, J=6.0, 8.8 Hz, 1H), 7.31 (d, J=2.4 Hz, 1H), 7.25 (t, J=9.6 Hz, 1H), 7.05 (br s, 1H), 5.54-5.22 (m, 1H), 4.77-4.68 (m, 2H), 4.47-4.33 (m, 2H), 3.99-3.85 (m, 1H), 3.73-3.59 (m, 1H), 3.56-3.36 (m, 3H), 3.25-3.02 (m, 2H), 2.80-2.63 (m, 2H), 2.51-2.30 (m, 3H), 2.24-2.03 (m, 5H), 2.00-1.83 (m, 2H), 1.75 (br t, J=8.4 Hz, 2H), 0.79 (br t, J=7.2 Hz, 3H); LCMS [ESI, M+1]: m/z=629.3.

### Example 560

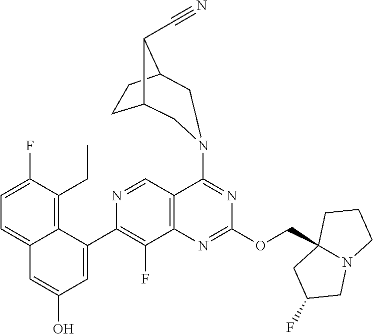 [SMILES: CCc1c(F)ccc2cc(O)cc(-c3ncc4c(N5CC6CCC(C5)C6C#N)nc(OC[C@@]56CCCN5C[C@H](F)C6)nc4c3F)c12]

### (1R,5S,8R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carbonitrile

### Example 561

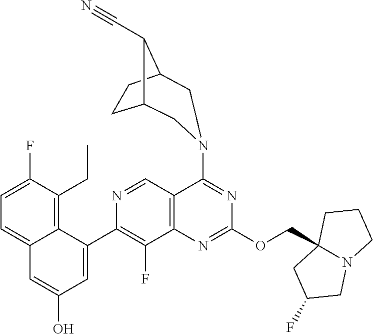 [SMILES: CCc1c(F)ccc2cc(O)cc(-c3ncc4c(N5CC6CCC(C5)C6C#N)nc(OC[C@@]56CCCN5C[C@H](F)C6)nc4c3F)c12]

### (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carbonitrile

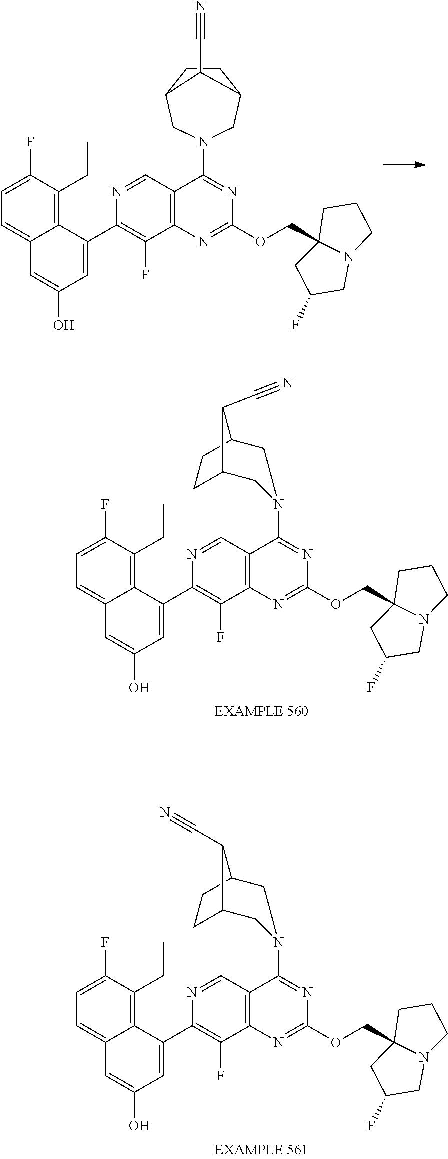 [SMILES: CCc1c(F)ccc2cc(O)cc(-c3ncc4c(N5CC6CCC(C5)C6C#N)nc(OC[C@@]56CCCN5C[C@H](F)C6)nc4c3F)c12 ; CCc1c(F)ccc2cc(O)cc(-c3ncc4c(N5CC6CCC(C5)C6C#N)nc(OC[C@@]56CCCN5C[C@H](F)C6)nc4c3F)c12 ; CCc1c(F)ccc2cc(O)cc(-c3ncc4c(N5CC6CCC(C5)C6C#N)nc(OC[C@@]56CCCN5C[C@H](F)C6)nc4c3F)c12]

3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-8-carbonitrile was purified by SFC (column: DAICEL CHIRALCEL OJ (250 mm×30 mm, 10 um); mobile phase: [0.1% NH₃H₂O ETOH]; B %: 50%-50%, 4.0 minutes) to afford Example 560 (5.41 mg, 13% yield) as a yellow solid. ¹H NMR (400 MHZ, CD₃OD) δ=9.11 (s, 1H), 7.64 (dd, J=6.0, 9.2 Hz, 1H), 7.26 (d, J=2.4 Hz, 1H), 7.22 (t, J=9.6 Hz, 1H), 7.04 (d, J=2.8 Hz, 1H), 5.39-5.21 (m, 1H), 4.79-4.62 (m, 2H), 4.36-4.17 (m, 2H), 3.98-3.86 (m, 2H), 3.29-3.13 (m, 3H), 3.05-2.97 (m, 2H), 2.68 (br s, 2H), 2.54-2.42 (m, 1H), 2.38-2.20 (m, 2H), 2.19-2.07 (m, 2H), 2.07-1.90 (m, 3H), 1.89-1.82 (m, 2H), 1.80-1.67 (m, 2H), 0.79 (br t, J=6.0 Hz, 3H). LCMS ([ESI, M+1):]: m/z=629.3. and Example 561 (13.8 mg, 33.8% yield) as a white solid. ¹H NMR (400 MHz, CD₃OD) δ=9.04 (s, 1H), 7.67 (dd, J=6.0, 9.6 Hz, 1H), 7.30 (d, J=2.8 Hz, 1H), 7.25 (t, J=9.6 Hz, 1H), 7.05 (d, J=2.8 Hz, 1H), 5.41-5.22 (m, 1H), 4.79-4.69 (m, 2H), 4.35-4.22 (m, 2H), 3.70-3.59 (m, 2H), 3.29-3.16 (m, 4H), 3.07-2.99 (m, 1H), 2.75 (br s, 2H), 2.54-2.42 (m, 1H), 2.40-2.20 (m, 2H), 2.19-2.10 (m, 2H), 2.06-1.96 (m, 4H), 1.95-1.87 (m, 1H), 1.81-1.73 (m, 2H), 0.79 (dt, J=2.0, 6.8 Hz, 3H). LCMS ([ESI, M+1):]: m/z=629.3.

### Example 562

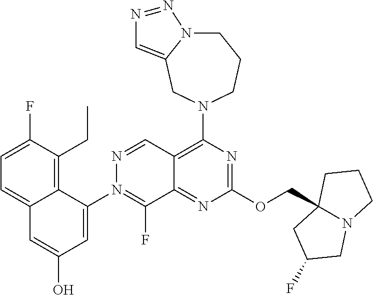

### 4-(4-(7,8-dihydro-4H-[1,2,3]triazolo[1,5-a][1,4]diazepin-5(6H)-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized according to the procedure described for example 541 to produce the desired compound as a white solid as white solid (0.09 formic acid salt); ¹H NMR (400 MHZ, METHANOL-d₄) δ=9.27-9.15 (m, 1H), 7.94-7.82 (m, 1H), 7.74-7.60 (m, 1H), 7.39-7.20 (m, 2H), 7.11-6.96 (m, 1H), 5.66-5.48 (m, 1H), 5.46 (s, 2H), 4.82-4.72 (m, 2H), 4.70-4.45 (m, 4H), 4.06-3.75 (m, 3H), 3.53-3.36 (m, 1H), 2.40 (br s, 6H), 2.38-2.25 (m, 2H), 2.23-2.07 (m, 2H), 0.84-0.70 (m, 3H); LCMS (ESI, M+1): m/z=631.3.

### Example 563

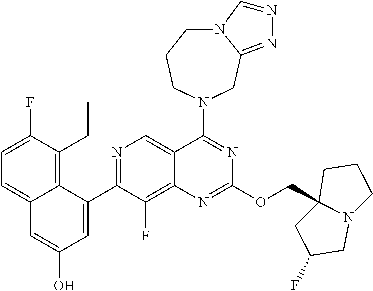 [SMILES: CCc1c(F)ccc2cc(O)cc(-c3ncc4c(N5CCCn6cnnc6C5)nc(OC[C@@]56CCCN5C[C@H](F)C6)nc4c3F)c12]

### 4-(4-(6,7-dihydro-5H-[1,2,4]triazolo[4,3-a][1,4]diazepin-8 (9H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

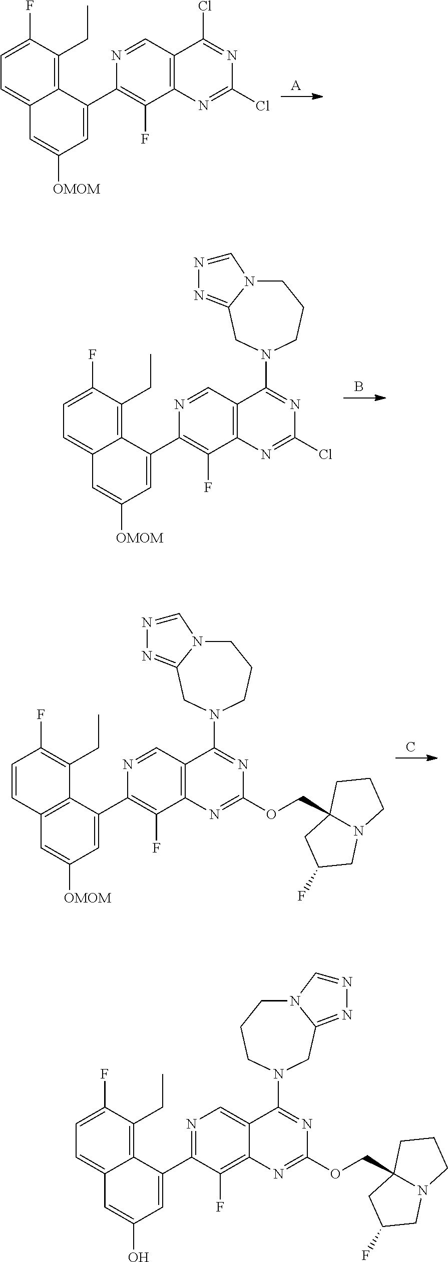

Step A. 8-(2-chloro-7-(8-ethyl-7-fluoro-3-(methoxymethoxy)naphthalen-1-yl)-8-fluoropyrido[4,3-d]pyrimidin-4-yl)-6,7,8,9-tetrahydro-5H-[1,2,4]triazolo[4,3-a][1,4]diazepine: A mixture of 2,4-dichloro-7-[8-ethyl-7-fluoro-3-(methoxymethoxy)-1-naphthyl]-8-fluoro-pyrido[4,3-d]pyrimidine (146 mg, 0.80 equiv) and 6,7,8,9-tetrahydro-5H-[1,2,4]triazolo[4,3-a][1,4]diazepine (56.0 mg, 1.0 equiv) in DMF (1.0 mL) was added DIPEA (157 mg, 3.0 equiv) and 4 Å molecular sieves (22.81 mg). The mixture was stirred at −40° C. for 0.2 hour. The mixture was purified by column chromatography [SiO₂, petroleum ether/ethyl acetate 1:1] to afford the tittle compound (200 mg, 89% yield) as a white solid. LCMS (ESI, M+1): m/z=552.1.

Step B. 8-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6,7,8,9-tetrahydro-5H-[1,2,4]triazolo[4,3-a][1,4]diazepine: A mixture of ((2R,7aS)-2-fluorohexahydro-1H-pyrrolizin-7a-yl)methanol (115 mg, 2.0 equiv) and 8-(2-chloro-7-(8-ethyl-7-fluoro-3-(methoxymethoxy)naphthalen-1-yl)-8-fluoropyrido[4,3-d]pyrimidin-4-yl)-6,7,8,9-tetrahydro-5H-[1,2,4]triazolo[4,3-a][1,4]diazepine (200 mg, 1.0 equiv) in dioxane (5.0 mL) was added DIPEA (140 mg, 3.0 equiv) and 4 Å molecular sieves (200 mg, 11 equiv). The mixture was stirred at 100° C. for 12 hours. The mixture was concentrated and purified by prep-HPLC [column: Waters Xbridge 150×25 mm×5 μm; phase: [water (ammonia hydroxide v/v)-ACN]; B %: 36%-66%, 9 minutes] to afford the title compound (50 mg, 20% yield) as a white solid. LCMS (ESI, M+1): m/z=675.2.

Step C. 4-(4-(6,7-dihydro-5H-[1.2.4]triazolo[4,3-a][1,4]diazepin-8 (9H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a solution of 8-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-6,7,8,9-tetrahydro-5H-[1,2,4]triazolo[4,3-a][1,4]diazepine (20.0 mg, 1.0 equiv) in dichloromethane (5.0 mL) was added HCl/MeOH (0.5 mL). The mixture was stirred at 0° C. for 1 hour. The mixture was concentrated and purified by prep-HPLC [column: waters Xbridge 150×25 mm×5 μm; mobile phase: [water (ammonia hydroxide v/v)-ACN]; B %: 27%-57%, 9 minutes] and lyophilized to afford the title compound (4.0 mg, 21% yield, 99% purity,) as a white oil (0.26 formic acid salt). ¹H NMR (400 MHZ, METHANOL-d₄) δ=9.17 (s, 1H), 8.41 (br s, 1H), 8.34 (s, 1H), 7.57 (dd, J=6.0, 9.2 Hz, 1H), 7.20 (d, J=2.4 Hz, 1H), 7.15 (t, J=9.2 Hz, 1H), 6.94 (d, J=2.0 Hz, 1H), 5.41-5.18 (m, 3H), 4.43-4.33 (m, 2H), 4.30-4.18 (m, 4H), 3.52-3.24 (m, 3H), 3.11-2.99 (m, 1H), 2.38-1.90 (m, 11H), 0.67 (t, J=7.2 Hz, 3H); LCMS (ESI, M+1): m/z=631.2.

### Example 564

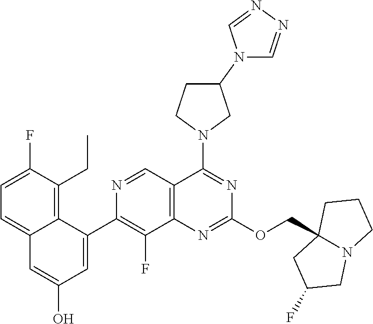 [SMILES: CCc1c(F)ccc2cc(O)cc(-c3ncc4c(N5CCC(n6cnnc6)C5)nc(OC[C@@]56CCCN5C[C@H](F)C6)nc4c3F)c12]

### 4-(4-(3-(4H-1,2,4-triazol-4-yl)pyrrolidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 544 to produce the desired compound as a yellow solid. ¹H NMR (400 MHZ, METHANOL-d4) δ=9.16 (s, 1H), 8.67 (s, 2H), 7.50 (dd, J=6.0, 8.8 Hz, 1H), 7.12 (d, J=2.4 Hz, 1H), 7.08 (t, J=9.2 Hz, 1H), 6.92 (d, J=2.8 Hz, 1H), 5.38-5.04 (m, 2H), 4.59-4.39 (m, 1H), 4.31-4.05 (m, 5H), 3.17-3.03 (m, 3H), 2.90 (dt, J=6.0, 9.2 Hz, 1H), 2.64 (br dd, J=4.8, 6.8 Hz, 1H), 2.55-2.32 (m, 2H), 2.29-2.07 (m, 2H), 2.02 (br dd, J=8.0, 9.6 Hz, 2H), 1.93-1.70 (m, 3H), 0.68 (br t, J=7.2 Hz, 3H); LCMS (ESI, M+1): m/z=631.3.

### Example 565

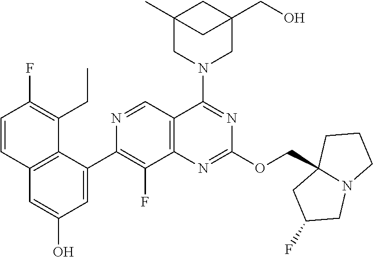 [SMILES: CCc1c(F)ccc2cc(O)cc(-c3ncc4c(N5CC6(C)CC(CO)(C5)C6)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(1-(hydroxymethyl)-5-methyl-3-azabicyclo[3.1.1]heptan-3-yl)pyrido[4,3-d]pyrimidin-7-yl)naphthalen-2-ol

The title compound was synthesized according to the procedure described for example 538 to produce the desired compound as a white solid. ¹H NMR (400 MHZ, METHANOL-d4) δ=9.49 (s, 1H), 7.68 (dd, J=6.0, 8.8 Hz, 1H), 7.35-7.20 (m, 2H), 7.06 (d, J=2.4 Hz, 1H), 5.57-5.25 (m, 1H), 4.65-4.37 (m, 3H), 4.28-3.98 (m, 4H), 3.72-3.38 (m, 4H), 3.25-3.04 (m, 1H), 2.55-2.25 (m, 4H), 2.20-1.96 (m, 4H), 1.75 (br d, J=6.8 Hz, 2H), 1.64 (br d, J=6.4 Hz, 2H), 1.27 (s, 3H), 0.80 (br t, J=7.2 Hz, 3H); LCMS (ESI, M+1): m/z=634.5

### Example 566

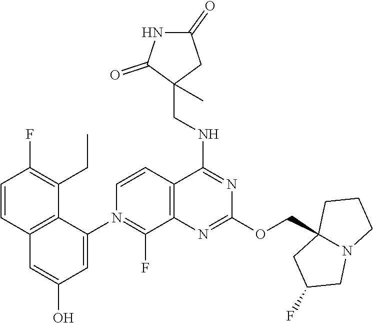

### 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-3-methylpyrrolidine-2,5-dione

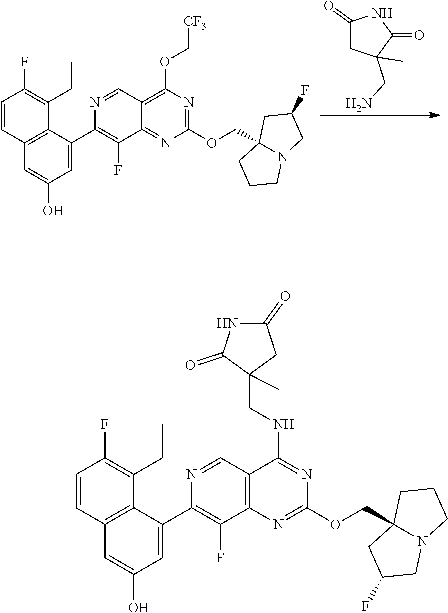

To a solution of 3-(aminomethyl)-3-methyl-pyrrolidine-2,5-dione (11.0 mg, 1.01 equiv, 2HCl) in DMF (1 mL) was added K₃PO₄ (53.7 mg, 5.0 equiv) and 4 Å molecular sieves (30 mg). The mixture was stirred at 40° C. for 0.5 hour. A solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30.0 mg, 1.0 equiv) in ACN (0.4 mL) was added. The mixture was stirred at 40° C. for 15.5 hours. The reaction mixture was diluted with DMF (1 mL) and filtered. The filtrate was purified by prep-HPLC [Phenomenex Luna C18 150×25 mm×10 um; A: water (FA), B: ACN, B %: 12%-42% over 11 min] and further re-purified by prep-HPLC [Waters Xbridge 150×25 mm×5 um; A: water (NH₄HCO₃), B: ACN, B %: 33%-63% over 9 min] and lyophilized to afford the title compound (3.95 mg, 12% yield) as an off-white solid; ¹H NMR (400 MHZ, METHANOL-d₄) δ=9.23 (s, 1H), 7.70 (dd, J=5.9, 9.0 Hz, 1H), 7.33 (d, J=2.6 Hz, 1H), 7.27 (t, J=9.4 Hz, 1H), 7.08-7.05 (m, 1H), 5.63-5.39 (m, 1H), 4.67-4.48 (m, 2H), 4.17 (dd, J=13.8, 15.7 Hz, 1H), 3.91-3.83 (m, 1H), 3.80 (br s, 3H), 3.08 (d, J=18.0 Hz, 1H), 2.66 (br d, J=18.0 Hz, 1H), 2.61-2.42 (m, 3H), 2.41-2.08 (m, 6H), 1.47 (s, 3H), 0.80 (br t, J=7.2 Hz, 3H); LCMS (ESI, M+1): m/z=635.4.

### Example 567

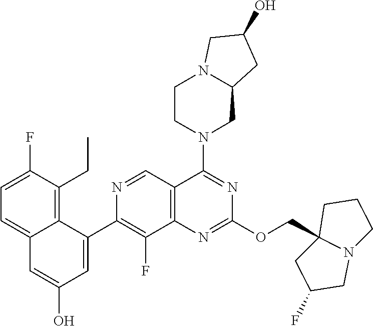 [SMILES: CCc1c(F)ccc2cc(O)cc(-c3ncc4c(N5CCN6C[C@@H](O)C[C@H]6C5)nc(OC[C@@]56CCCN5C[C@H](F)C6)nc4c3F)c12]

### (7S,8aS)-2-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) octahydropyrrolo[1,2-a]pyrazin-7-ol

The title compound was synthesized according to the procedure described for example 544 to produce the desired compound as a white solid. ¹H NMR (400 MHZ, METHANOL-d₄) δ=9.07 (s, 1H), 7.67 (dd, J=6.0, 8.8 Hz, 1H), 7.30 (d, J=2.4 Hz, 1H), 7.25 (t, J=9.2 Hz, 1H), 7.04 (dd, J=2.4, 6.8 Hz, 1H), 5.41-5.17 (m, 1H), 4.77 (br d, J=12.4 Hz, 1H), 4.68 (br d, J=13.2 Hz, 1H), 4.41-4.34 (m, 1H), 4.33-4.19 (m, 2H), 3.66 (br t, J=12.4 Hz, 1H), 3.43-3.34 (m, 1H), 3.28-3.14 (m, 4H), 3.03 (br d, J=10.0 Hz, 2H), 2.50-2.36 (m, 4H), 2.33-2.10 (m, 5H), 2.03-1.87 (m, 3H), 1.56-1.41 (m, 1H), 0.79 (dt, J=2.4, 7.2 Hz, 3H); LCMS (ESI, M+1): m/z=635.4

### Example 568

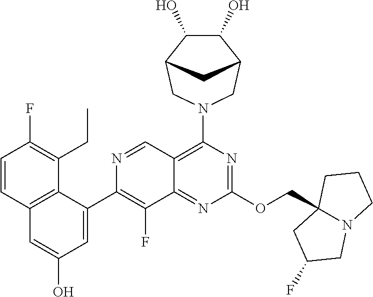 [SMILES: CCc1c(F)ccc2cc(O)cc(-c3ncc4c(N5C[C@@H]6C[C@H](C5)[C@H](O)[C@@H]6O)nc(OC[C@@]56CCCN5C[C@H](F)C6)nc4c3F)c12]

### (1R,5S,6R,7S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-6,7-diol

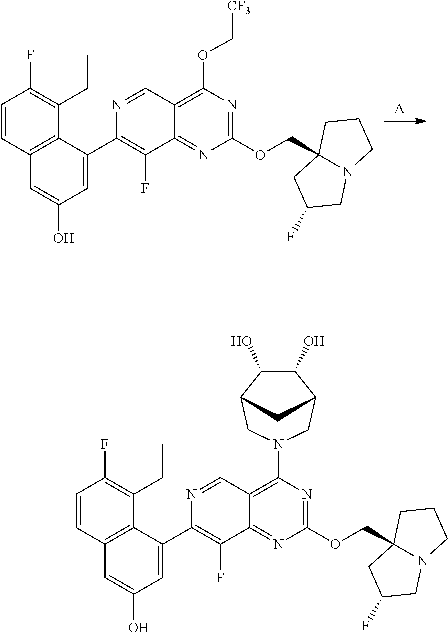

To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (100 mg, 1.0 equiv) in DMF (0.5 mL) and DCM (0.5 mL) were added K₃PO₄ (107 mg, 3.0 equiv), (1S,5R,6S,7R)-3-azabicyclo[3.2.1]octane-6,7-diol (purchased from Enamine Ltd) (46 mg, 1.5 eq, HCl) and 4 Å molecular sieves (20 mg). The mixture was stirred at 40° C. for 12 hours. The reaction mixture was filtered and purified by prep-HPLC [Phenomenex Synergi C18 150×25 mm×10 μm; A: water (10 mM FA); B: ACN; B %: 11%-41% over 10 min] to afford the title compound (25.9 mg, 24% yield) as a white solid (0.2 formic acid salt). ¹H NMR (400 MHZ, METHANOL-d₄) δ=9.27 (s, 1H), 7.69-7.66 (m, 1H), 7.31 (d, J=2.4 Hz, 1H), 7.25 (t, J=9.6 Hz, 1H), 7.06 (d, J=2.4 Hz, 1H), 5.55-5.35 (m, 1H), 4.58-4.42 (m, 2H), 4.21-4.13 (m, 2H), 3.59 (d, J=6.4 Hz, 6H), 3.30-3.19 (m, 2H), 2.62-2.49 (m, 1H), 2.48-2.37 (m, 4H), 2.33-2.25 (m, 1H), 2.23-2.12 (m, 3H), 2.10-2.02 (m, 1H), 1.93 (d, J=12.4 Hz, 1H), 1.73-1.63 (m, 1H), 0.83-0.76 (m, 3H); LCMS (ESI, M+1): m/z=636.3.

### Example 569

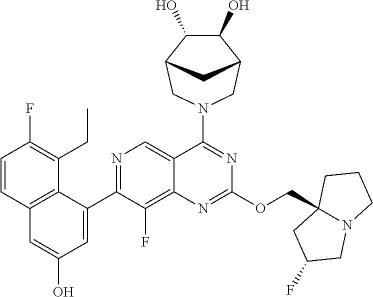 [SMILES: CCc1c(F)ccc2cc(O)cc(-c3ncc4c(N5C[C@H]6C[C@@H](C5)[C@H](O)[C@H]6O)nc(OC[C@@]56CCCN5C[C@H](F)C6)nc4c3F)c12]

### (1R,5S,6S,7S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-6,7-diol (stereochemistry was arbitrarily assigned)

The title compound was synthesized from Intermediate 5 peak 3 and 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol according to the procedure described for example 568 to produce the desired compound as a yellow solid. ¹H NMR (400 MHZ, dimethylsulfoxide-d₆) δ=9.43-9.14 (m, 1H), 7.70 (dd, J=6.2, 8.8 Hz, 1H), 7.30 (br t, J=9.6 Hz, 1H), 7.25 (d, J=2.0 Hz, 1H), 7.06-6.93 (m, 1H), 5.41-5.16 (m, 1H), 5.02-4.69 (m, 3H), 4.52-4.42 (m, 1H), 4.22-4.08 (m, 1H), 4.01 (dd, J=4.0, 10.4 Hz, 1H), 3.82 (br t, J=5.2 Hz, 1H), 3.76-3.63 (m, 2H), 3.24 (br d, J=13.2 Hz, 1H), 3.09 (br d, J=9.2 Hz, 2H), 3.01 (br s, 1H), 2.86-2.79 (m, 1H), 2.25 (br s, 1H), 2.20-1.90 (m, 7H), 1.89-1.74 (m, 3H), 1.69 (br dd, J=4.8, 11.6 Hz, 1H), 0.70 (t, J=7.2 Hz, 3H); ¹H NMR (400 MHZ, chloroform-d) δ=9.22-8.84 (m, 1H), 7.58-7.41 (m, 1H), 7.21-7.02 (m, 2H), 6.98-6.57 (m, 1H), 5.38-5.08 (m, 1H), 4.87-4.10 (m, 5H), 4.00-3.65 (m, 2H), 3.55-3.37 (m, 1H), 3.29 (br s, 2H), 3.23-2.92 (m, 4H), 2.52-2.02 (m, 8H), 1.91-1.74 (m, 3H), 1.61-1.51 (m, 1H), 1.17-1.05 (m, 3H); ¹⁹F NMR (400 MHZ, dimethylsulfoxide-d₆) δ=−139.353, −172.075; LCMS (ESI, M+1): m/z=636.4.

### Example 570

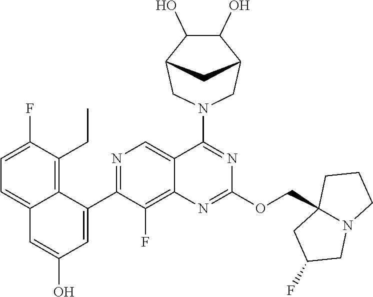 [SMILES: CCc1c(F)ccc2cc(O)cc(-c3ncc4c(N5C[C@H]6C[C@@H](C5)C(O)C6O)nc(OC[C@@]56CCCN5C[C@H](F)C6)nc4c3F)c12]

### (1R,5S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-6,7-diol (Stereochemistry was Arbitrarily Assigned)

The title compound was synthesized from Intermediate 5 peak 4 and 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol according to the procedure described for example 568 to produce the desired compound as a white solid. ¹H NMR (400 MHZ, dimethylsulfoxide-d₆) δ=9.97-9.90 (m, 1H), 9.11-9.06 (m, 1H), 7.80-7.73 (m, 1H), 7.39-7.30 (m, 2H), 7.04-7.00 (m, 1H), 5.38-5.19 (m, 1H), 4.91-4.84 (m, 1H), 4.71-4.62 (m, 1H), 4.47-4.38 (m, 1H), 4.28-4.18 (m, 1H), 4.17-4.10 (m, 1H), 4.07-4.02 (m, 2H), 4.01-3.94 (m, 1H), 3.61-3.44 (m, 2H), 3.15-3.05 (m, 2H), 3.02-2.99 (m, 1H), 2.88-2.79 (m, 1H), 2.26-2.20 (m, 1H), 2.20-2.11 (m, 2H), 2.11-1.96 (m, 3H), 1.95-1.85 (m, 2H), 1.85-1.73 (m, 4H), 0.75-0.69 (m, 3H); ¹⁹F NMR (400 MHz, dimethylsulfoxide-d₆) δ=−119.629, −139.308, −172.150; LCMS (ESI, M+1): m/z=636.1.

### Example 571

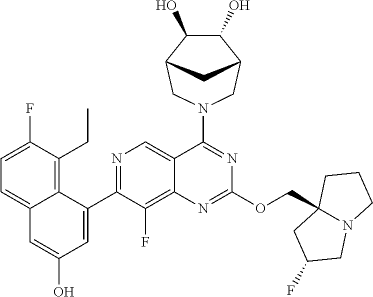 [SMILES: CCc1c(F)ccc2cc(O)cc(-c3ncc4c(N5C[C@H]6C[C@@H](C5)[C@@H](O)[C@@H]6O)nc(OC[C@@]56CCCN5C[C@H](F)C6)nc4c3F)c12]

### (1R,5S,6R,7R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-6,7-diol (Stereochemistry was Arbitrarily Assigned)

The title compound was synthesized from Intermediate 5 peak 1 and 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol according to the procedure described for example 568 to produce the desired compound as a white solid. ¹H NMR (400 MHZ, dimethylsulfoxide-d₆) δ=9.93-9.86 (m, 1H), 9.33-9.24 (m, 1H), 7.79-7.73 (m, 1H), 7.38-7.30 (m, 2H), 7.05-6.99 (m, 1H), 5.37-5.18 (m, 1H), 5.02-4.69 (m, 3H), 4.52-4.42 (m, 1H), 4.15-4.09 (m, 1H), 4.07-4.00 (m, 1H), 3.86-3.79 (m, 1H), 3.75-3.65 (m, 2H), 3.28-3.22 (m, 1H), 3.15-3.06 (m, 2H), 3.03-2.99 (m, 1H), 2.86-2.79 (m, 1H), 2.34-2.31 (m, 1H), 2.28-2.22 (m, 1H), 2.15-2.04 (m, 4H), 2.02-1.91 (m, 2H), 1.89-1.74 (m, 3H), 1.72-1.65 (m, 1H), 0.75-0.68 (m, 3H); ¹⁹F NMR (400 MHz, dimethylsulfoxide-d₆) δ=−119.622, −139.405, −172.127; LCMS (ESI, M+1): m/z=636.7.

### Example 572

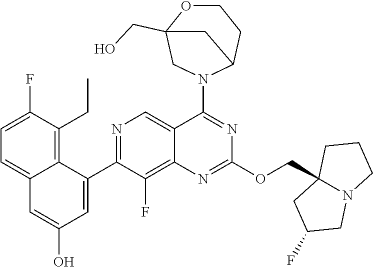 [SMILES: CCc1c(F)ccc2cc(O)cc(-c3ncc4c(N5CC6(CO)CC5CCO6)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(1-(hydroxymethyl)-2-oxa-6-azabicyclo[3.2.1]octan-6-yl)pyrido[4,3-d]pyrimidin-7-yl)naphthalen-2-ol

The title compound was synthesized according to the procedure described for example 538 to produce the desired compound as a yellow solid (0.34 formic acid salt). ¹H NMR (400 MHZ, CD₃OD) δ=9.56-9.11 (m, 1H), 7.68 (dd, J=6.0, 8.8 Hz, 1H), 7.31 (d, J=2.8 Hz, 1H), 7.25 (t, J=9.6 Hz, 1H), 7.05 (s, 1H), 5.57-5.27 (m, 2H), 4.50-4.30 (m, 3H), 4.28-4.13 (m, 1H), 4.05-3.95 (m, 1H), 3.92-3.80 (m, 1H), 3.78-3.69 (m, 2H), 3.46-3.38 (m, 2H), 3.20-3.09 (m, 1H), 2.58-2.39 (m, 2H), 2.38-2.25 (m, 2H), 2.24-2.18 (m, 2H), 2.15-2.03 (m, 4H), 2.02-1.90 (m, 2H), 1.89-1.77 (m, 1H), 0.84-0.77 (m, 3H); LCMS (ESI, M+1): m/z=636.3.

### Example 573

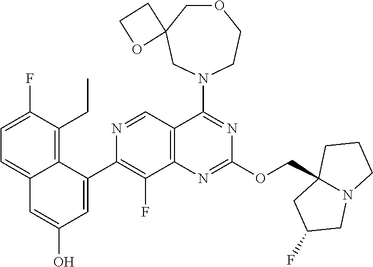 [SMILES: CCc1c(F)ccc2cc(O)cc(-c3ncc4c(N5CCOCC6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1,6-dioxa-9-azaspiro[3.6]decan-9-yl)pyrido[4,3-d]pyrimidin-7-yl)naphthalen-2-ol

The title compound was synthesized according to the procedure described for example 544 to produce the desired compound as a white solid (0.22 formic acid salt). ¹H NMR (400 MHZ, CD₃OD) δ=9.56 (s, 1H), 7.68 (dd, J=6.0, 8.8 Hz, 1H), 7.34-7.21 (m, 2H), 7.06 (dd, J=2.4, 8.8 Hz, 1H), 5.46-5.24 (m, 1H), 4.70-4.51 (m, 4H), 4.44-3.95 (m, 8H), 3.92-3.81 (m, 1H), 3.14-3.07 (m, 1H), 2.76-2.66 (m, 1H), 2.63-2.37 (m, 3H), 2.35-2.13 (m, 4H), 2.11-2.03 (m, 2H), 1.99-1.88 (m, 1H), 0.86-0.75 (m, 3H); LCMS (ESI, M+1): m/z=636.4.

### Example 574

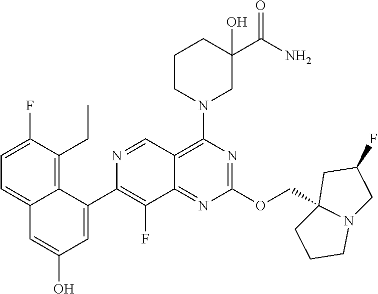 [SMILES: CCc1c(F)ccc2cc(O)cc(-c3ncc4c(N5CCCC(O)(C(N)=O)C5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-hydroxypiperidine-3-carboxamide

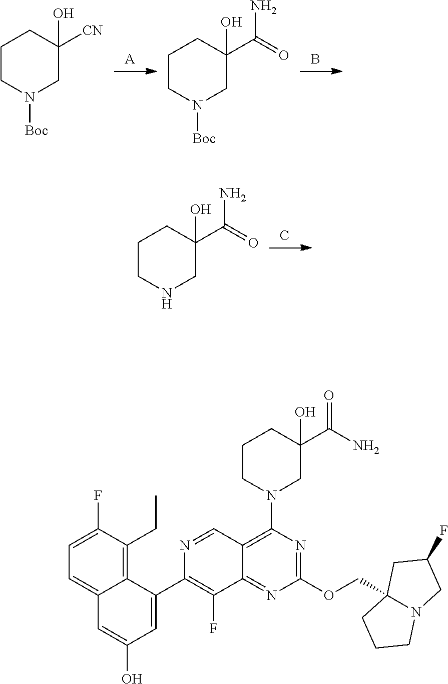

Step A. tert-butyl 3-carbamoyl-3-hydroxypiperidine-1-carboxylate: To a solution of tert-butyl 3-cyano-3-hydroxy-piperidine-1-carboxylate (4.00 g, 1 equiv) in DCM (50 mL) was added H₂SO₄ (9.54 g, 5.5 equiv) at 0° C. The reaction mixture was stirred at 0° C. for 2 hours. The pH of the mixture was adjusted with aq. NaOH (7.00 g, 9.9 equiv, 40% in water) to ˜7. (Boc)₂O (19.3 g, 5.0 equiv) was added. The reaction mixture was stirred at 20° C. for 16 hours. The reaction mixture was diluted with DCM (50 mL) and water (50 mL), extracted with DCM (50 mL). The combined organic layers were washed with brine (30 mL), dried over anhydrous sodium sulfate, concentrated and purified by reversed phase flash chromatography [water (0.1% FA)/acetonitrile] to afford the title compound (3.9 g, 43% yield) as a yellow oil; ¹H NMR (400 MHZ, CHLOROFORM-d) δ=6.86 (br s, 1H), 4.04-3.84 (m, 2H), 3.27 (br d, J=14.0 Hz, 1H), 3.10 (br d, J=6.4 Hz, 1H), 2.83 (br t, J=12.6 Hz, 1H), 2.12-1.96 (m, 1H), 1.81-1.62 (m, 2H), 1.61-1.52 (m, 1H), 1.47 (s, 9H), 1.35 (s, 9H); LCMS (ESI, M+1): m/z=245.3.

Step B. 3-hydroxypiperidine-3-carboxamide: To a solution of tert-butyl 3-carbamoyl-3-hydroxy-piperidine-1-carboxylate (1.00 g, 1.0 equiv) in MeCN (5 mL) was added HCl·dioxane (4 M, 10 mL). The reaction mixture was stirred at 20° C. for 1 hour. The mixture was concentrated under vacuum to afford the title compound (590 mg, crude, HCl) as a yellow solid.

Step C. 1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-hydroxypiperidine-3-carboxamide: To a solution of 3-hydroxypiperidine-3-carboxamide (157 mg, 2 equiv, HCl), 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (257 mg, 1.0 equiv) and DIEA (280 mg, 5.0 equiv) in DMF (1 mL) was added 4 Å molecular sieve (10 mg). The reaction mixture was stirred at 40° C. for 16 hours. The mixture was filtered and the filtrate was purified by prep-HPLC [column: Unisil 3-100 C18 Ultra 150×50 mm×3 um; A: water (FA), B: ACN, B %: 8%-38% over 7 min], followed by prep-HPLC [column: Welch Ultimate XB-SiOH 250×50×10 um; A: Hexane, B: EtOH, B %: 10%-50% over 15 min] and lyophilized to afford the title compound (8.91 mg, 3.2% yield) as a white solid (0.50 formic acid salt); ¹HNMR (400 MHZ, METHANOL-d4) δ=9.23 (t, J=2.8 Hz, 1H), 7.68 (dd, J=6.0, 9.2 Hz, 1H), 7.31 (d, J=2.8 Hz, 1H), 7.25 (t, J=9.4 Hz, 1H), 7.06 (t, J=2.8 Hz, 1H), 5.49-5.24 (m, 1H), 4.71 (br d, J=13.2 Hz, 1H), 4.61-4.29 (m, 3H), 3.94-3.84 (m, 1H), 3.52-3.33 (m, 4H), 3.17-3.04 (m, 1H), 2.52-2.27 (m, 4H), 2.26-2.13 (m, 3H), 2.11-2.02 (m, 2H), 2.01-1.83 (m, 3H), 0.85-0.73 (m, 3H); LCMS (ESI, M+1): m/z=637.3.

### Example 575

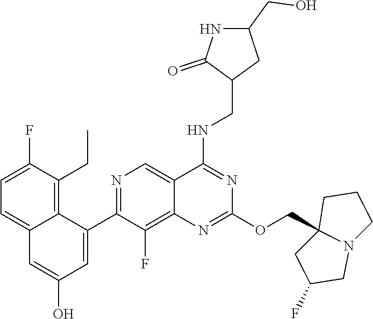 [SMILES: CCc1c(F)ccc2cc(O)cc(-c3ncc4c(NCC5CC(CO)NC5=O)nc(OC[C@@]56CCCN5C[C@H](F)C6)nc4c3F)c12]

### 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-5-(hydroxymethyl)pyrrolidin-2-one

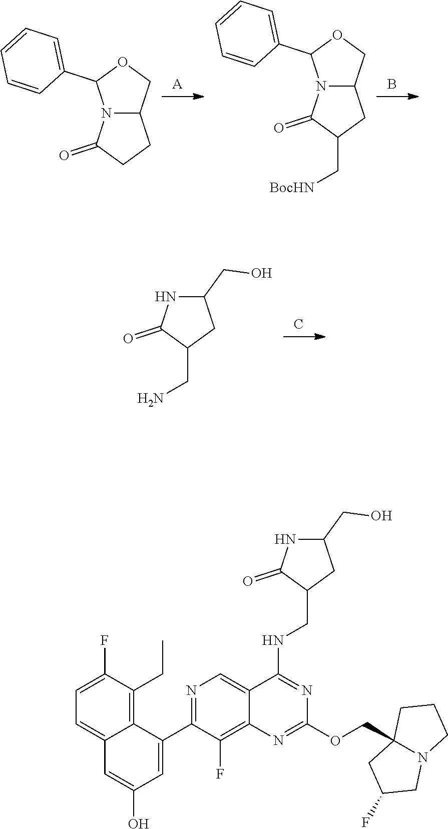

Step A. tert-butyl ((5-oxo-3-phenyltetrahydro-1H,3H-pyrrolo[1,2-c]oxazol-6-yl)methyl)carbamate: A mixture of tert-butyl N-(benzenesulfonylmethyl)carbamate (293.71 mg, 1.1 equiv) in THF (15 mL) was degassed and purged with N₂ for 3 times, and then LiHMDS (1 M, 2.07 mL, 2.1 equiv) was added to the mixture before it was stirred at −78° C. for 30 min under N₂ atmosphere. 3-phenyltetrahydro-3H,5H-pyrrolo[1,2-c]oxazol-5-one (200 mg, 1.0 equiv) in THF (3 mL) was added to the mixture and the resulting was stirred at −78° C. for 30 min under N₂ atmosphere. The reaction mixture was quenched by addition of aqueous NH₄Cl (5 mL) at −78° C., and then diluted with water 30 mL, extracted with ethyl acetate 60 mL (20 mL×3). The combined organic layers were dried over Na₂SO₄, filtered and purified by column chromatography (SiO₂, Petroleum ether/Ethyl acetate 1:1) to afford the title compound (100 mg, 30.6% yield) as a yellow oil. ¹H NMR (400 MHZ, DMSO-d₆) δ=1.38 (s, 9H) 1.59-1.76 (m, 1H) 2.44 (br s, 2H) 2.95-3.06 (m, 2H) 3.43-3.50 (m, 1H) 4.07-4.16 (m, 1H) 4.17-4.24 (m, 1H) 6.04-6.10 (m, 1H) 6.77-6.91 (m, 1H) 7.30-7.44 (m, 5H).

Step B. 3-(aminomethyl)-5-(hydroxymethyl)pyrrolidin-2-one: To a solution of tert-butyl ((5-oxo-3-phenyltetrahydro-1H,3H-pyrrolo[1,2-c]oxazol-6-yl)methyl)carbamate (100 mg, 1.0 equiv) in dioxane (3.0 mL) was added HCl/dioxane (4 M, 1.13 mL, 15.0 equiv). The mixture was stirred at 25° C. for 2 hours. The reaction mixture was filtered to give a white solid, which was used into the next step without further purification.

Step C. 3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-5-(hydroxymethyl)pyrrolidin-2-one: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (80 mg, 1.0 equiv), 3-(aminomethyl)-5-(hydroxymethyl)pyrrolidin-2-one (40 mg, 1.6 equiv) and CH₃CN (1.0 mL) in DMF (1 mL) was added K₃PO₄ (286 mg, 1.35 mmol, 10 equiv) and 4 Å molecular sieves (100 mg, 25 equiv). The mixture was stirred at 60° C. for 2 hours. The reaction mixture was filtered and purified by prep-HPLC [Waters Xbridge 150×25 mm×5 um; mobile phase: [water (ammonia hydroxide v/v)-ACN]; B %: 44%-74%, 9 min] to afford the title compound (6.1 mg, 6.71% yield) as a white solid; ¹H NMR (400 MHZ, METHANOL-d₄) δ=0.81 (br t, J=7.6 Hz, 3H) 1.77-1.90 (m, 1H) 1.92-2.07 (m, 3H) 2.11-2.29 (m, 4H) 2.37 (br d, J=4.4 Hz, 1H) 2.40-2.54 (m, 2H) 2.99-3.13 (m, 2H) 3.15-3.29 (m, 3H) 3.47-3.58 (m, 2H) 3.67 (dd, J=11.2, 4.4 Hz, 1H) 3.77 (br d, J=4.4 Hz, 1H) 3.86-3.98 (m, 1H) 3.99-4.08 (m, 1H) 4.29-4.39 (m, 2H) 5.26 (br s, 1H) 7.06 (s, 1H) 7.23-7.35 (m, 2H) 7.69 (dd, J=9.2, 6.0 Hz, 1H) 9.16 (s, 1H); LCMS (ESI, M+1): m/z=637.3.

### Example 576

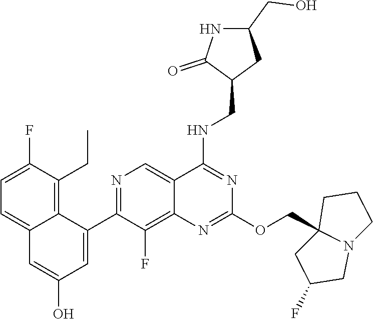 [SMILES: CCc1c(F)ccc2cc(O)cc(-c3ncc4c(NC[C@@H]5C[C@H](CO)NC5=O)nc(OC[C@@]56CCCN5C[C@H](F)C6)nc4c3F)c12]

### (3S,5R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-5-(hydroxymethyl)pyrrolidin-2-one

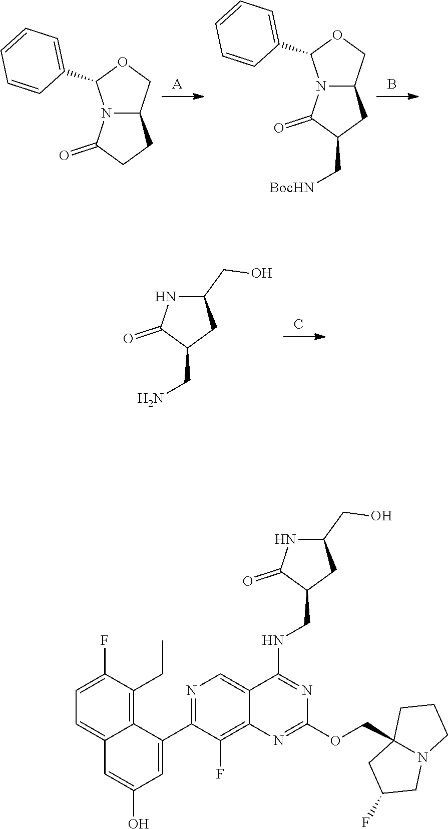

Step A. (((3S,6S,7aR)-5-oxo-3-phenyltetrahydro-1H,3H-pyrrolo[1,2-c]oxazol-6-yl)methyl)carbamate: A mixture of tert-butyl N-(p-tolylsulfonylmethyl)carbamate (232 mg, 1.1 equiv) in THF (3.0 mL) was degassed and purged with nitrogen for 3 times, and then LDA (2 M, 2.0 equiv) was added. Then the mixture was stirred at −78° C. for 30 minutes under nitrogen atmosphere. And then (3S,7aR)-3-phenyltetrahydropyrrolo[1,2-c]oxazol-5 (3H)-one (150 mg, 1.0 equiv) in THF (3.0 mL) was added to the mixture and stirred at −78° C. for 30 minutes under nitrogen atmosphere. The mixture was quenched with aqueous NH₄Cl (5.0 mL) at −78° C. and extracted with ethyl acetate (3×20 mL). The combined organic layers were dried over Na₂SO₄, filtered, concentrated and purified with column chromatography [SiO₂, Petroleum ether/Ethyl acetate 1:1] to afford the title compound (30.0 mg, 12% yield) as a yellow oil; LCMS (ESI, M−55): m/z=277.0

Step B. (3S,5R)-3-(aminomethyl)-5-(hydroxymethyl)pyrrolidin-2-one: To a solution of (((3S,6S,7aR)-5-oxo-3-phenyltetrahydro-1H,3H-pyrrolo[1,2-c]oxazol-6-yl)methyl)carbamate (30.0 mg, 1.0 equiv) in ACN (1.0 mL) was added HCl/dioxane (4.0 M, 1.0 mL). The mixture was stirred at 25° C. for 6 hours. The reaction mixture was concentrated and purified by prep-TLC [SiO₂, DCM/MeOH 10/1] to afford the title compound (10.0 mg, 76% yield) as a yellow solid.

Step C. (3S,5R)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-5-(hydroxymethyl)pyrrolidin-2-one: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (10.0 mg, 1.0 equiv) in dimethyl formamide (1.0 mL) were added K₃PO₄ (20.0 mg, 1.0 equiv), (3S,5R)-3-(aminomethyl)-5-(hydroxymethyl)pyrrolidin-2-one (5.00 mg, 2.5 equiv) and 4 Å molecular sieves (10.0 mg). The mixture was stirred at 60° C. for 2 hours. The mixture was filtered, concentrated and purified by prep-HPLC [column: Waters Xbridge 150×25 mm×5 um; mobile phase: water (ammonia hydroxide v/v)-ACN; B %: 22%-52%, 9 min] and lyophilized to afford a title compound (2.70 mg, 24% yield) as yellow solid; ¹H NMR (400 MHZ, DMSO-d₆) δ=9.92 (br s, 1H), 9.30 (s, 1H), 7.83-7.73 (m, 2H), 7.38-7.31 (m, 2H), 7.00 (d, J=1.2 Hz, 1H), 5.48-5.09 (m, 1H), 4.94-4.71 (m, 1H), 4.21-4.03 (m, 2H), 3.97-3.86 (m, 1H), 3.62-3.50 (m, 2H), 3.15-2.99 (m, 3H), 2.91-2.77 (m, 2H), 2.73-2.62 (m, 1H), 2.36-2.30 (m, 2H), 2.15-1.99 (m, 4H), 1.88-1.75 (m, 3H), 1.66-1.56 (m, 1H), 1.23 (s, 1H), 1.05 (t, J=7.2 Hz, 1H), 0.71 (br t, J=7.2 Hz, 3H), LCMS (ESI, M+1): m/z=637.2

### Example 577

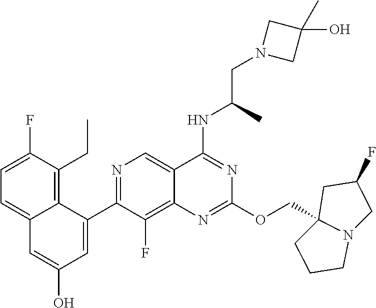 [SMILES: CCc1c(F)ccc2cc(O)cc(-c3ncc4c(N[C@H](C)CN5CC(C)(O)C5)nc(OC[C@@]56CCCN5C[C@H](F)C6)nc4c3F)c12]

### 1-((R)-2-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino) propyl)-3-methylazetidin-3-ol

The title compound was synthesized according to the procedure (except for no 4A Molecular sieves were added) described for example 544 to produce the desired compound as a white solid (1 formic acid salt); ¹H NMR (400 MHZ, METHANOL-d₄) δ=9.24 (s, 1H), 8.40 (br s, 1H), 7.69 (dd, J=6.0, 8.8 Hz, 1H), 7.32 (d, J=2.4 Hz, 1H), 7.29-7.22 (m, 1H), 7.09-7.02 (m, 1H), 5.65-5.25 (m, 1H), 4.83-4.70 (m, 1H), 4.65-4.53 (m, 2H), 4.19-3.51 (m, 8H), 3.24-3.12 (m, 1H), 2.71-2.31 (m, 4H), 2.39-2.20 (m, 1H), 2.29-2.04 (m, 4H), 1.49 (d, J=5.6 Hz, 3H), 1.42 (dd, J=4.4, 6.6 Hz, 3H), 0.90-0.66 (m, 3H); LCMS (ESI, M+1): m/z=637.4.

### Example 578

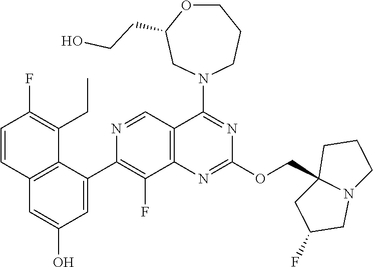 [SMILES: CCc1c(F)ccc2cc(O)cc(-c3ncc4c(N5CCCO[C@@H](CCO)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-((S)-2-(2-hydroxyethyl)-1,4-oxazepan-4-yl)pyrido[4,3-d]pyrimidin-7-yl)naphthalen-2-ol

The title compound was synthesized according to the procedure described for example 538 to produce the desired compound as a brown solid; ¹H NMR (400 MHZ, METHANOL-d₄) δ=9.14 (d, J=4.4 Hz, 1H), 7.67 (dd, J=5.6, 9.2 Hz, 1H), 7.30 (d, J=2.8 Hz, 1H), 7.25 (t, J=9.2 Hz, 1H), 7.06 (dd, J=2.8, 9.6 Hz, 1H), 5.45-5.14 (m, 1H), 4.56 (br t, J=14.8 Hz, 1H), 4.37-4.20 (m, 4H), 4.17-4.03 (m, 2H), 3.75 (br t, J=5.2 Hz, 2H), 3.70-3.56 (m, 2H), 3.28-3.12 (m, 3H), 3.08-2.96 (m, 1H), 2.55-2.42 (m, 1H), 2.35-2.08 (m, 6H), 2.04-1.77 (m, 5H), 0.85-0.71 (m, 3H); LCMS (ESI, M+1): m/z=638.3.

### Example 579

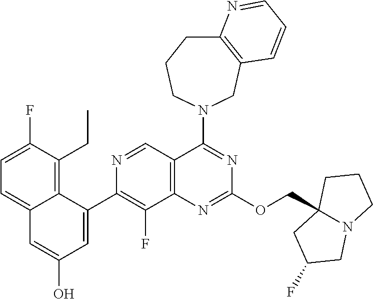 [SMILES: CCc1c(F)ccc2cc(O)cc(-c3ncc4c(N5CCCc6ncccc6C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(5,7,8,9-tetrahydro-6H-pyrido[3,2-c]azepin-6-yl)pyrido[4,3-d]pyrimidin-7-yl)naphthalen-2-ol

The title compound was synthesized according to the procedure described for example 541 to produce the desired compound as a white solid (0.88 formic acid salt); ¹H NMR (400 MHZ, METHANOL-d4) δ=9.17 (s, 1H), 8.37 (dd, J=1.2, 4.0 Hz, 1H), 7.96 (d, J=7.6 Hz, 1H), 7.68 (dd, J=6.0, 9.2 Hz, 1H), 7.36-7.29 (m, 2H), 7.25 (t, J=9.2 Hz, 1H), 7.04 (d, J=2.8 Hz, 1H), 5.57-5.37 (m, 1H), 5.25-5.21 (m, 2H), 4.51-4.33 (m, 4H), 3.81-3.56 (m, 3H), 3.30-3.19 (m, 3H), 2.60-2.06 (m, 10H), 0.77 (t, J=7.6 Hz, 3H). LCMS (ESI, M+1): m/z=641.4.

### Example 580

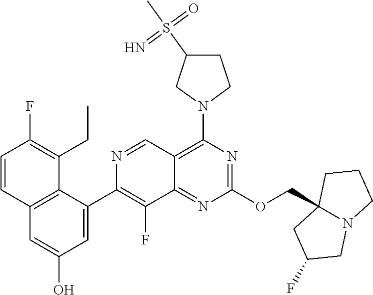 [SMILES: CCc1c(F)ccc2cc(O)cc(-c3ncc4c(N5CCC(S(C)(=N)=O)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy) 4-(3-(S-methylsulfonimidoyl)pyrrolidin-1-yl)pyrido[4,3-d]pyrimidin-7-yl)naphthalen-2-ol

The title compound was synthesized according to the procedure described for example 538 to produce the desired compound as an off-white solid; ¹H NMR (400 MHZ, METHANOL-d₄) δ=9.26 (s, 1H), 7.62 (br dd, J=6.0, 8.8 Hz, 1H), 7.24 (d, J=2.4 Hz, 1H), 7.19 (t, J=9.6 Hz, 1H), 7.04 (br d, J=2.4 Hz, 1H), 5.41-5.20 (m, 1H), 4.45 (br s, 2H), 4.34 (br dd, J=4.0, 10.4 Hz, 2H), 4.30-4.24 (m, 1H), 4.22-4.04 (m, 2H), 3.27-3.15 (m, 3H), 3.12 (s, 3H), 3.04-2.97 (m, 1H), 2.61 (br d, J=4.0 Hz, 2H), 2.45 (br d, J=4.8 Hz, 1H), 2.39-2.19 (m, 2H), 2.17-2.07 (m, 2H), 2.02-1.85 (m, 3H), 0.78 (br t, J=7.2 Hz, 3H); LCMS (ESI, M+1): m/z=641.3.

### Example 581

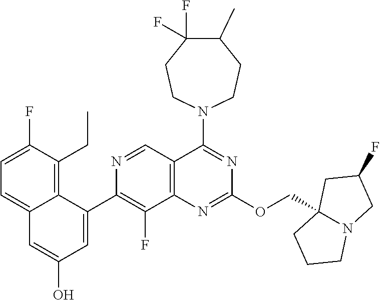 [SMILES: CCc1c(F)ccc2cc(O)cc(-c3ncc4c(N5CCC(C)C(F)(F)CC5)nc(OC[C@@]56CCCN5C[C@H](F)C6)nc4c3F)c12]

### 4-(4-(4,4-difluoro-5-methylazepan-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 544 to produce the desired compound as a white solid. ¹H NMR (400 MHZ, METHANOL-d4) δ=9.16 (s, 1H), 7.67 (dd, J=5.6, 8.8 Hz, 1H), 7.30 (d, J=2.4 Hz, 1H), 7.25 (s, 1H), 7.05 (br s, 1H), 5.50-5.15 (m, 1H), 4.44-3.89 (m, 7H), 3.17-2.98 (m, 1H), 2.64-2.41 (m, 3H), 2.37-1.67 (m, 12H), 1.12-1.00 (m, 3H), 0.84-0.75 (m, 3H); LCMS (ESI, M+1): m/z=642.3.

### Example 582

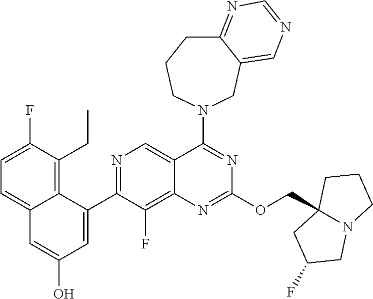 [SMILES: CCc1c(F)ccc2cc(O)cc(-c3ncc4c(N5CCCc6ncncc6C5)nc(OC[C@@]56CCCN5C[C@H](F)C6)nc4c3F)c12]

### 4-(4-(8,9-dihydro-5H-pyrimido[5,4-c]azepin-6(7H)-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized using Intermediate 6 according to the procedure described for Example 541 to produce the desired compound as a white solid (0.3 formic acid salt); ¹H NMR (400 MHZ, METHANOL-d₄) δ=9.19 (s, 1H), 8.95 (s, 1H), 8.89 (s, 1H), 7.68 (dd, J=5.6, 9.2 Hz, 1H), 7.33-7.21 (m, 2H), 7.04 (d, J=2.8 Hz, 1H), 5.48-5.30 (m, 1H), 5.27-5.13 (m, 2H), 4.54-4.38 (m, 2H), 4.35-4.19 (m, 2H), 3.56-3.38 (m, 3H), 3.27-3.10 (m, 3H), 2.54-2.27 (m, 5H), 2.26-2.07 (m, 4H), 2.05-1.91 (m, 1H), 0.78 (t, J=7.2 Hz, 3H); LCMS (ESI, M+1): m/z=642.3.

### Example 583

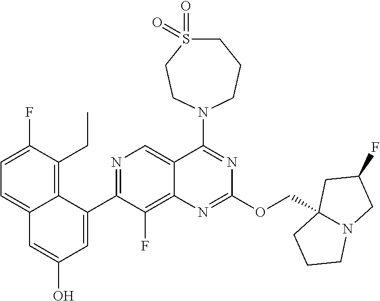 [SMILES: CCc1c(F)ccc2cc(O)cc(-c3ncc4c(N5CCCS(=O)(=O)CC5)nc(OC[C@@]56CCCN5C[C@H](F)C6)nc4c3F)c12]

### 4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1,4-thiazepane 1,1-dioxide

The title compound was synthesized according to the procedure described for example 541 to produce the desired compound as a white solid (0.3 formic acid salt). ¹H NMR (400 MHZ, METHANOL-d₄) δ=9.20 (s, 1H), 7.68 (dd, J=6.0, 9.2 Hz, 1H), 7.31 (d, J=2.8 Hz, 1H), 7.26 (t, J=9.2 Hz, 1H), 7.05 (d, J=2.8 Hz, 1H), 5.51-5.26 (m, 1H), 4.54-4.20 (m, 6H), 3.73 (brt, J=5.2 Hz, 2H), 3.61-3.34 (m, 5H), 3.24-3.12 (m, 1H), 2.56-2.45 (m, 3H), 2.44-2.29 (m, 2H), 2.27-2.05 (m, 4H), 2.05-1.95 (m, 1H), 0.86-0.71 (m, 3H); (ESI, M+1): m/z=642.3.

### Example 584

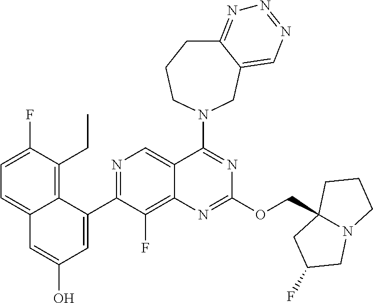 [SMILES: CCc1c(F)ccc2cc(O)cc(-c3ncc4c(N5CCCc6nnncc6C5)nc(OC[C@@]56CCCN5C[C@H](F)C6)nc4c3F)c12]

### 4-(4-(8,9-dihydro-5H-[1,2,3]triazino[5,4-c]azepin-6(7H)-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized using Intermediate 7 according to the procedure described for example 541 to produce the desired compound as a yellow solid; ¹H NMR (400 MHZ, METHANOL-d₄) δ=9.32 (s, 1H), 9.19 (s, 1H), 7.68 (dd, J=6.0, 9.2 Hz, 1H), 7.30 (s, 1H), 7.25 (t, J=9.6 Hz, 1H), 7.07-7.01 (m, 1H), 5.42-5.22 (m, 1H), 5.22-5.10 (m, 1H), 4.60-4.13 (m, 3H), 4.10-3.95 (m, 1H), 3.49-3.33 (m, 2H), 3.13 (br s, 4H), 3.09-2.95 (m, 1H), 2.55-2.41 (m, 2H), 2.39 (br s, 1H), 2.22-2.10 (m, 3H), 2.08-1.95 (m, 3H), 1.94-1.80 (m, 1H), 0.84-0.73 (m, 3H); LCMS (ESI, M+1): m/z=643.1.

### Example 585

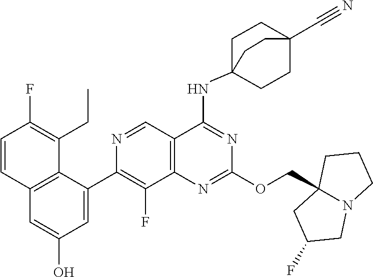 [SMILES: CCc1c(F)ccc2cc(O)cc(-c3ncc4c(NC56CCC(C#N)(CC5)CC6)nc(OC[C@@]56CCCN5C[C@H](F)C6)nc4c3F)c12]

### 4-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)bicyclo[2.2.2]octane-1-carbonitrile

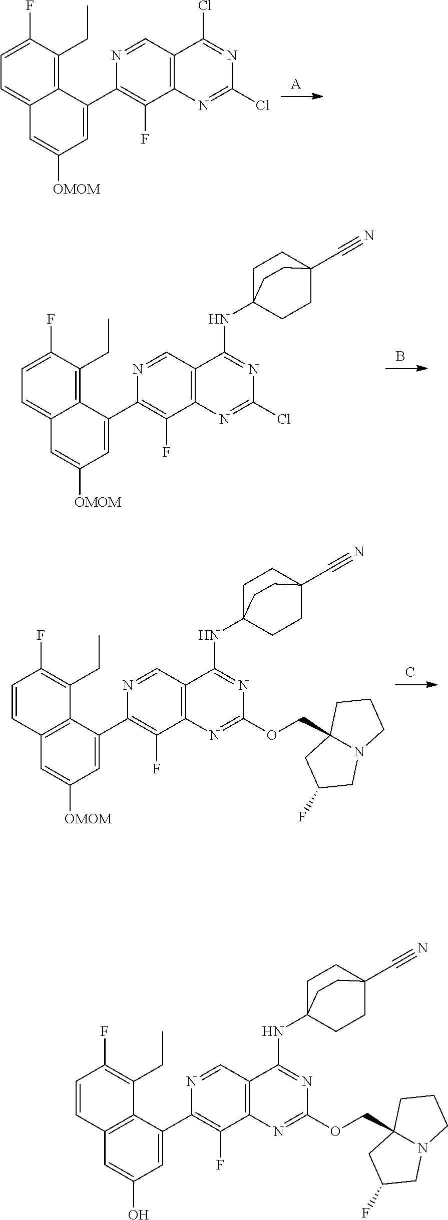

Step A. 4-((2-chloro-7-(8-ethyl-7-fluoro-3-(methoxymethoxy)naphthalen-1-yl)-8-fluoropyrido[4,3-d]pyrimidin-4-yl)amino)bicyclo[2.2.2]octane-1-carbonitrile: To a solution of 2,4-dichloro-7-(8-ethyl-7-fluoro-3-(methoxymethoxy)naphthalen-1-yl)-8-fluoropyrido[4,3-d]pyrimidine (15 mg, 1.0 equiv) and excess 4-aminobicyclo[2.2.2]octane-1-carbonitrile in DMF (3.0 mL) was added DIPEA (38.7 mg, 3.0 equiv) and 4 Å molecular sieves (45 mg, 9.0 equiv). The mixture was stirred at −40° C. for 0.2 hour. The mixture was diluted with ethyl acetate (30 mL) and filtered. The filtrate was washed with brine (30 mL×2), dried over anhydrous sodium sulfate, filtered and concentrated under reduced pressure to afford the title compound (70 mg, crude). The crude product was used for the next step without further purification; LCMS (ESI, M+1): m/z=564.2

Step B. 4-((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)bicyclo[2.2.2]octane-1-carbonitrile: To a solution of 4-((2-chloro-7-(8-ethyl-7-fluoro-3-(methoxymethoxy)naphthalen-1-yl)-8-fluoropyrido[4,3-d]pyrimidin-4-yl)amino)bicyclo[2.2.2]octane-1-carbonitrile (55 mg, 1.0 equiv) and ((2R,7aS)-2-fluorohexahydro-1H-pyrrolizin-7a-yl)methanol (46.6 mg, 3.0 equiv) in THF (3 mL) were added DIPEA (63 mg, 5.0 equiv) and 4 Å molecular sieves (10 mg, 2.1 equiv). The mixture was stirred at 60° C. for 12 hours and then at 70° C. for 5 hours. The mixture was diluted with ethyl acetate (30 mL) and filtered. The filtrate was washed with brine (20 mL×3). The organic phase was filtered, concentrated and purified by prep-HPLC (column: Phenomenex C18 75×30 mm×3 um; mobile phase: [water (FA)-ACN]; B %: 20%-50%, 7 min) to afford the title compound (25 mg, 35% yield) as a white solid; LCMS (ESI, M+1): m/z=687.2

Step C. 4-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)bicyclo[2.2.2]octane-1-carbonitrile: To a solution of 4-((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)bicyclo[2.2.2]octane-1-carbonitrile (20 mg, 1.0 equiv) in MeOH (1.5 mL) was added HCl/MeOH (4 M, 1.5 mL, 206 equiv). The mixture was stirred at 0° C. for 0.5 hour. The mixture was concentrated and purified by prep-HPLC (column: Phenomenex luna C18 150×25 mm×10 um; mobile phase: [water (FA)-ACN]; B %: 16%-46%, 10 min) and lyophilized to afford the title compound (3.8 mg, 20% yield,) as a white solid; ¹H NMR (400 MHZ, METHANOL-d4) δ=9.27 (s, 1H), 7.67 (dd, J=5.6, 9.2 Hz, 1H), 7.30 (d, J=2.8 Hz, 1H), 7.24 (t, J=9.2 Hz, 1H), 7.02 (d, J=2.4 Hz, 1H), 5.54-5.26 (m, 1H), 4.52-4.26 (m, 2H), 3.58-3.38 (m, 3H), 3.16 (dt, J=6.4, 9.6 Hz, 1H), 2.48-2.42 (m, 1H), 2.41-2.31 (m, 8H), 2.25-2.07 (m, 11H), 2.05-1.91 (m, 1H), 0.77 (t, J=7.2 Hz, 3H); LCMS (ESI, M+1): m/z=643.4

### Example 586

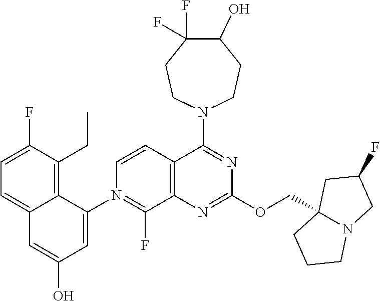

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5,5-difluoroazepan-4-ol

The title compound was synthesized according to the procedure described for example 538 to produce the desired compound as a yellow solid. ¹H NMR (400 MHZ, METHANOL-d4) δ=0.74-0.86 (m, 3H) 1.44 (t, J=7.2 Hz, 2H) 1.89 (br s, 1H) 2.00 (br s, 2H) 2.11-2.28 (m, 4H) 2.31-2.41 (m, 2H) 2.42-2.54 (m, 1H) 2.70-2.90 (m, 1H) 2.96-3.05 (m, 1H) 3.16-3.26 (m, 2H) 3.43 (q, J=7.2 Hz, 1H) 3.87-3.96 (m, 1H) 4.06 (br s, 1H) 4.20-4.34 (m, 4H) 5.18-5.41 (m, 1H) 7.06 (dd, J=7.6, 2.4 Hz, 1H) 7.18-7.34 (m, 2H) 7.66 (dd, J=8.8, 5.6 Hz, 1H) 9.17 (d, J=4.0 Hz, 1H); LCMS (ESI, M+1): m/z=644.2.

### Example 587

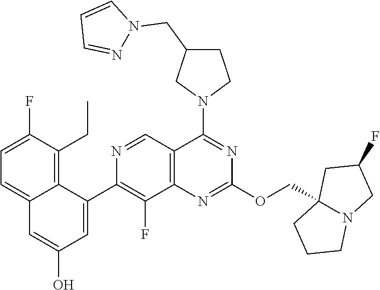 [SMILES: CCc1c(F)ccc2cc(O)cc(-c3ncc4c(N5CCC(Cn6cccn6)C5)nc(OC[C@@]56CCCN5C[C@H](F)C6)nc4c3F)c12]

### 4-(4-(3-((1H-pyrazol-1-yl)methyl)pyrrolidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 541 to produce the desired compound as a white solid (0.44 formic acid salt). ¹H NMR (400 MHZ, DMSO-d6) δ 0.73 (br t, J=6.8 Hz, 3H) 1.76-1.88 (m, 4H) 1.99-2.16 (m, 6H) 2.31-2.36 (m, 1H) 2.80-2.91 (m, 3H) 3.10 (br d, J=8.4 Hz, 2H) 4.06 (br d, J=10.4 Hz, 2H) 4.16 (br dd, J=10.4, 2.44 Hz, 2H) 4.25-4.36 (m, 3H) 5.19-5.38 (m, 1H) 6.27 (br d, J=2.0 Hz, 1H) 7.01 (br d, J=2.8 Hz, 1H) 7.30-7.40 (m, 2H) 7.48 (br s, 1H) 7.73-7.86 (m, 2H) 8.18 (s, 1H) 9.26 (s, 1H); LCMS (ESI, M+1): m/z=644.6.

### Example 588

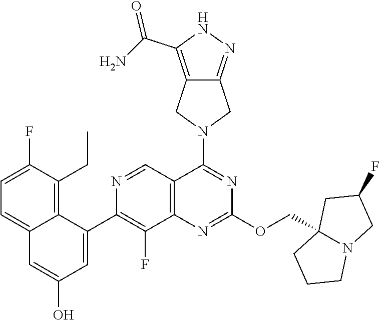 [SMILES: CCc1c(F)ccc2cc(O)cc(-c3ncc4c(N5Cc6n[nH]c(C(N)=O)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,4,5,6-tetrahydropyrrolo[3,4-c]pyrazole-3-carboxamide

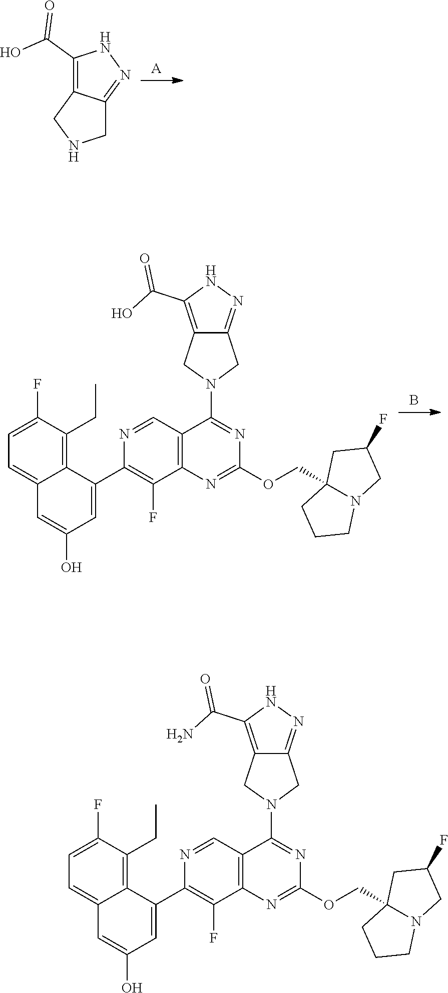

Step A 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,4,5,6-tetrahydropyrrolo[3,4-c]pyrazole-3-carboxylic acid: To a solution of 2,4,5,6-tetrahydropyrrolo[3,4-c]pyrazole-3-carboxylic acid (25.0 mg, 1.1 equiv) in dimethyl formamide (1.0 mL) were added K₃PO₄ (94.5 mg, 3.0 equiv), 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (87.9 mg, 1.0 equiv) and 4 Å molecular sieves (10.0 mg). The mixture was stirred at 60° C. for 2 hours. The reaction mixture was partitioned between ethyl acetate (40 mL) and water (30 mL). The organic phase was separated and dried over Na₂SO₄. Then it was filtered and concentrated under reduced pressure to give a yellow solid; LCMS (ESI, M+1): m/z=646.1.

Step B 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,4,5,6-tetrahydropyrrolo[3,4-c]pyrazole-3-carboxamide: To a solution of 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,4,5,6-tetrahydropyrrolo[3,4-c]pyrazole-3-carboxylic acid (30 mg, 1 equiv) in DMF (1 mL) were added DIPEA (12.01 mg, 2 equiv) NH₄Cl (24.8 mg, 10 equiv) and HATU (53.0 mg, 3 equiv). The mixture was stirred at 25° C. for 1 hr. The reaction mixture was concentrated and purified with prep-HPLC [column: Waters Xbridge 150×25 mm×5 um; mobile phase: [water (ammonia hydroxide v/v)-ACN]; B %: 28%-58%, 8.5 min] to afford the title compound (5.6 mg, 18% yield) as a gray solid (0.6 formic acid salt); ¹H NMR (400 MHZ, DMSO-d₆) δ=9.73-9.28 (m, 1H), 8.24 (s, 1H), 7.82-7.57 (m, 3H), 7.41-7.30 (m, 2H), 7.03 (d, J=1.6 Hz, 1H), 5.49-5.20 (m, 3H), 5.15-4.90 (m, 2H), 4.25-4.10 (m, 2H), 3.13-3.06 (m, 2H), 2.84 (br d, J=6.0 Hz, 2H), 2.40-2.33 (m, 1H), 2.20-2.02 (m, 4H), 1.88-1.75 (m, 3H), 0.74 (br t, J=7.2 Hz, 3H), LCMS (ESI, M+1): m/z=645.3

### Example 589

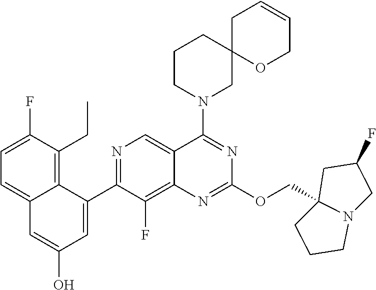 [SMILES: CCc1c(F)ccc2cc(O)cc(-c3ncc4c(N5CCCC6(CC=CCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-oxa-8-azaspiro[5.5]undec-3-en-8-yl)pyrido[4,3-d]pyrimidin-7-yl)naphthalen-2-ol

The title compound was synthesized according to the procedure described for example 538 to produce the desired compound as a yellow solid; ¹H NMR (400 MHZ, METHANOL-d4) δ=9.24-9.14 (m, 1H), 7.68 (dd, J=6.0, 9.2 Hz, 1H), 7.30 (d, J=2.4 Hz, 1H), 7.25 (t, J=9.6 Hz, 1H), 7.06 (dd, J=2.8, 8.0 Hz, 1H), 5.83-5.67 (m, 2H), 5.42-5.24 (m, 1H), 4.79-4.47 (m, 4H), 4.37-4.23 (m, 2H), 4.06 (br d, J=18.0 Hz, 1H), 3.94-3.82 (m, 1H), 3.70-3.58 (m, 1H), 3.23-3.18 (m, 1H), 3.09-2.97 (m, 1H), 2.54-2.31 (m, 2H), 2.29-2.18 (m, 2H), 2.17-2.05 (m, 5H), 2.04-1.98 (m, 2H), 1.95-1.87 (m, 1H), 1.80-1.64 (m, 2H), 1.39-1.23 (m, 1H), 0.80 (br t, J=7.2 Hz, 3H). LCMS (ESI, M+1): m/z=646.4,

### Example 590

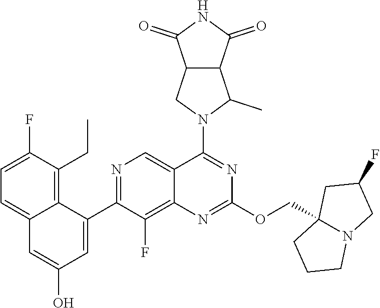 [SMILES: CCc1c(F)ccc2cc(O)cc(-c3ncc4c(N5CC6C(=O)NC(=O)C6C5C)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-4-methyltetrahydropyrrolo[3,4-c]pyrrole-1,3(2H,3aH)-dione

The title compound was synthesized using Intermediate 8 according to the procedure described for example 541 to produce the desired compound as a white solid; (0.10 formic acid salt). SFC: Chiralcel OD-3 50×4.6 mm I.D., 3 μm, Isocratic elution: 40% methanol+ACN (0.05% DEA) in CO₂, 3 mL/min, t_{R}=0.553 min, 1.073 min; ¹H NMR (400 MHz, methanol-d₄) δ=9.18 (d, J=2.4 Hz, 1H), 7.76-7.59 (m, 1H), 7.31 (d, J=2.4 Hz, 1H), 7.25 (t, J=9.2 Hz, 1H), 7.08-7.04 (m, 1H), 5.55-5.22 (m, 1H), 4.75-4.43 (m, 3H), 4.38-4.24 (m, 2H), 3.90-3.50 (m, 2H), 3.43-3.34 (m, 2H), 3.07-2.98 (m, 1H), 2.54-2.11 (m, 7H), 1.96-1.86 (m, 1H), 1.66-1.50 (m, 4H), 0.84-0.76 (m, 3H); ¹⁹F NMR (400 MHZ, methanol-d₄) δ=−121.084, −138.457, −73.675; LCMS (ESI, M+1): m/z=647.3.

### Example 591

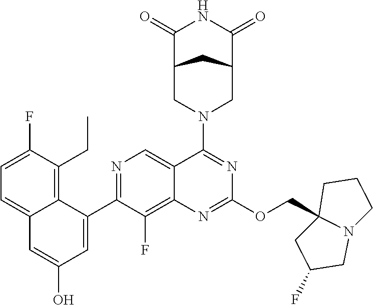 [SMILES: CCc1c(F)ccc2cc(O)cc(-c3ncc4c(N5C[C@H]6C[C@@H](C5)C(=O)NC6=O)nc(OC[C@@]56CCCN5C[C@H](F)C6)nc4c3F)c12]

### (1R,5S)-7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3,7-diazabicyclo[3.3.1]nonane-2,4-dione

The title compound was synthesized using Intermediate 9 according to the procedure described for example 541 to produce the desired compound as an off-white solid (0.1 formic acid salt); ¹H NMR (400 MHZ, METHANOL-d₄) δ=9.04 (s, 1H), 7.70-7.66 (m, 1H), 7.34-7.19 (m, 2H), 7.07-7.06 (m, 1H), 5.45-5.31 (m, 1H), 5.01-4.97 (m, 1H), 4.42-4.25 (m, 2H), 4.01-3.78 (m, 2H), 3.55-3.33 (m, 3H), 3.16-3.07 (m, 1H), 2.95-2.93 (m, 2H), 2.58-2.36 (m, 3H), 2.35-2.15 (m, 4H), 2.10-2.01 (m, 4H), 0.79-0.75 (m, 3H); LCMS (ESI, M+1): m/z=647.4.

### Example 592

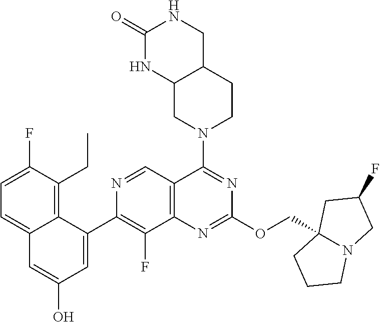 [SMILES: CCc1c(F)ccc2cc(O)cc(-c3ncc4c(N5CCC6CNC(=O)NC6C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)octahydropyrido[3,4-d]pyrimidin-2(1H)-one

The title compound was synthesized according to the procedure described for example 538 to produce the desired compound as a yellow solid. ¹H NMR (400 MHZ, METHANOL-d4) δ=9.05 (s, 1H), 7.65 (dd, J=6.0, 8.8 Hz, 1H), 7.30-7.19 (m, 2H), 7.04 (d, J=2.8 Hz, 1H), 5.45-5.13 (m, 1H), 4.56-4.37 (m, 2H), 4.36-4.21 (m, 2H), 3.93-3.70 (m, 3H), 3.44 (dd, J=4.2, 12.4 Hz, 1H), 3.28-3.13 (m, 4H), 3.05-2.96 (m, 1H), 2.57-2.43 (m, 1H), 2.41-2.26 (m, 2H), 2.23-2.09 (m, 3H), 2.08-1.94 (m, 3H), 1.93-1.78 (m, 2H), 0.85-0.72 (m, 3H). LCMS (ESI, M+1): m/z=648.2.

### Example 593

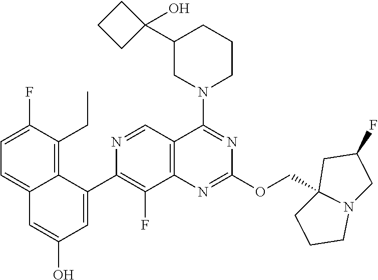 [SMILES: CCc1c(F)ccc2cc(O)cc(-c3ncc4c(N5CCCC(C6(O)CCC6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(3-(1-hydroxycyclobutyl) piperidin-1-yl)pyrido[4,3-d]pyrimidin-7-yl)naphthalen-2-ol

The title compound was synthesized according to the procedure described for example 544 to produce the desired compound as a white solid. ¹H NMR (400 MHZ, METHANOL-d4) δ=9.05 (s, 1H), 7.66 (dd, J=6.0, 8.8 Hz, 1H), 7.29 (d, J=2.8 Hz, 1H), 7.23 (t, J=9.2 Hz, 1H), 7.06 (dd, J=2.8, 4.4 Hz, 1H), 5.45-5.16 (m, 1H), 4.82-4.65 (m, 2H), 4.38-4.18 (m, 2H), 3.27-2.92 (m, 6H), 2.62-2.41 (m, 2H), 2.37-2.14 (m, 6H), 2.10-2.01 (m, 4H), 1.89-1.58 (m, 7H), 1.41-1.24 (m, 1H), 0.87-0.70 (m, 3H); LCMS (ESI, M+1): m/z=648.2.

### Example 594

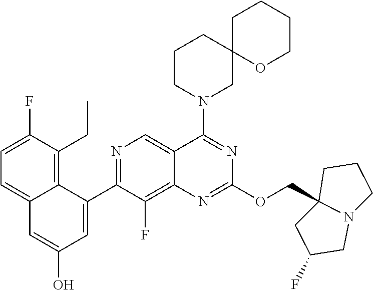 [SMILES: CCc1c(F)ccc2cc(O)cc(-c3ncc4c(N5CCCC6(CCCCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-oxa-8-azaspiro[5.5]undecan-8-yl)pyrido[4,3-d]pyrimidin-7-yl)naphthalen-2-ol

The title compound was synthesized according to the procedure described for example 538 to produce the desired compound as a yellow solid (0.29 formic acid salt); ¹H NMR (400 MHZ, methanol-d₄) δ=9.38-9.12 (m, 1H), 8.57 (s, 1H), 7.69 (dd, J=6.0, 8.8 Hz, 1H), 7.31 (d, J=2.0 Hz, 1H), 7.26 (t, J=9.2 Hz, 1H), 7.09 (br dd, J=2.4, 5.2 Hz, 1H), 5.49-5.13 (m, 1H), 4.74-4.49 (m, 2H), 4.39-4.19 (m, 2H), 3.69-3.55 (m, 2H), 3.50-3.41 (m, 1H), 3.22 (br d, J=20.0 Hz, 2H), 3.09-2.95 (m, 1H), 2.57-2.21 (m, 5H), 2.17-1.88 (m, 6H), 1.77-1.42 (m, 9H), 0.82 (br t, J=7.2 Hz, 3H); LCMS (ESI, M+1): m/z=648.3.

### Example 595

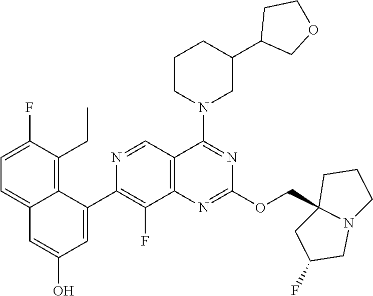 [SMILES: CCc1c(F)ccc2cc(O)cc(-c3ncc4c(N5CCCC(C6CCOC6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(3-(tetrahydrofuran-3-yl) piperidin-1-yl)pyrido[4,3-d]pyrimidin-7-yl)naphthalen-2-ol

The title compound was synthesized according to the procedure described for example 538 to produce the desired compound as a yellow solid; ¹H NMR (400 MHZ, METHANOL-d₄) δ=9.02 (dd, J=1.6, 6.0 Hz, 1H), 7.67 (dd, J=6.0, 8.8 Hz, 1H), 7.30 (d, J=2.4 Hz, 1H), 7.24 (t, J=9.2 Hz, 1H), 7.14-6.98 (m, 1H), 5.39-5.22 (m, 1H), 4.71-4.47 (m, 2H), 4.36-4.20 (m, 2H), 4.02-3.95 (m, 1H), 3.92-3.83 (m, 2H), 3.77-3.71 (m, 1H), 3.55-3.49 (m, 1H), 3.43-3.36 (m, 1H), 3.24-3.18 (m, 2H), 3.08-2.95 (m, 2H), 2.69-2.53 (m, 1H), 2.50-2.34 (m, 2H), 2.26-2.18 (m, 2H), 2.14-2.10 (m, 2H), 1.96 (br dd, J=8.8, 16.8 Hz, 3H), 1.79-1.68 (m, 3H), 1.60-1.47 (m, 2H), 1.34-1.14 (m, 1H), 0.89-0.73 (m, 3H). LCMS (ESI, M+1): m/z=648.2.

### Example 596

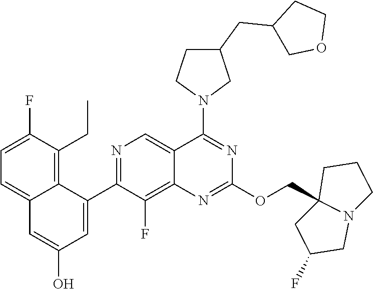 [SMILES: CCc1c(F)ccc2cc(O)cc(-c3ncc4c(N5CCC(CC6CCOC6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(3-((tetrahydrofuran-3-yl)methyl)pyrrolidin-1-yl)pyrido[4,3-d]pyrimidin-7-yl)naphthalen-2-ol

The title compound was synthesized according to the procedure described for example 538 to produce the desired compound as a white solid (0.1 formic acid salt); ¹H NMR (400 MHZ, DMSO-d₆) δ=9.29 (d, J=5.6 Hz, 1H), 7.77 (dd, J=6.0, 8.8 Hz, 1H), 7.49-7.29 (m, 2H), 7.08 (m, 1H), 5.10 (m, 1H), 4.28-3.99 (m, 5H), 3.92-3.57 (m, 5H), 3.16-2.95 (m, 4H), 2.83 (br d, J=6.2 Hz, 3H), 2.45-1.94 (m, 11H), 1.89-1.37 (m, 8H), 0.73 (q, J=7.6 Hz, 3H); LCMS (ESI, M+1): m/z=648.4.

### Example 597

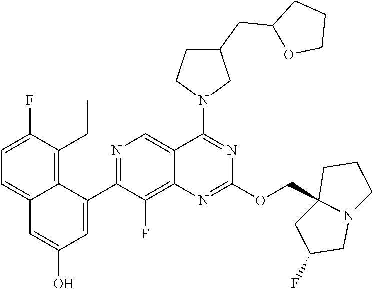 [SMILES: CCc1c(F)ccc2cc(O)cc(-c3ncc4c(N5CCC(CC6CCCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(3-((tetrahydrofuran-2-yl)methyl)pyrrolidin-1-yl)pyrido[4,3-d]pyrimidin-7-yl)naphthalen-2-ol

The title compound was synthesized according to the procedure described for example 538 to produce the desired compound as an off-white solid (0.2 formic acid salt); ¹HNMR (400 MHZ, METHANOL-d₄) δ ppm=0.80 (q, J=7.6 Hz, 3H) 1.48-1.60 (m, 1H) 1.68-1.85 (m, 3H) 1.94 (br d, J=6.4 Hz, 3H) 1.99-2.06 (m, 2H) 2.07-2.20 (m, 3H) 2.20-2.41 (m, 3H) 2.41-2.60 (m, 2H) 3.00-3.11 (m, 1H) 3.20-3.31 (m, 3H) 3.33-3.46 (m, 1H) 3.68-3.74 (m, 1H) 3.68-3.92 (m, 3H) 3.92-4.02 (m, 1H) 4.03-4.25 (m, 2H) 4.26-4.33 (m, 1H) 4.34-4.43 (m, 1H) 5.23-5.44 (m, 1H) 7.05 (dd, J=6.8, 2.32 Hz, 1H) 7.25 (t, J=9.4 Hz, 1H) 7.30 (d, J=2.4 Hz, 1H) 7.68 (dd, J=9.2, 5.6 Hz, 1H) 8.49-8.59 (m, 1H) 9.26 (d, J=4.4 Hz, 1H). LCMS (ESI, M+1): m/z=648.4

### Example 598

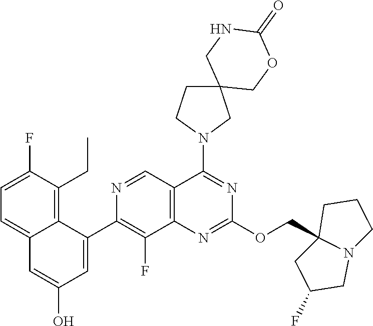 [SMILES: CCc1c(F)ccc2cc(O)cc(-c3ncc4c(N5CCC6(CNC(=O)OC6)C5)nc(OC[C@@]56CCCN5C[C@H](F)C6)nc4c3F)c12]

### 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-7-oxa-2,9-diazaspiro[4.5]decan-8-one

The title compound was synthesized according to the procedure described for example 544 to produce the desired compound as a white solid; ¹H NMR (400 MHZ, CD₃OD) δ=9.26 (s, 1H), 7.67 (dd, J=6.0, 8.8 Hz, 1H), 7.29 (d, J=2.4 Hz, 1H), 7.24 (t, J=9.6 Hz, 1H), 7.04 (d, J=2.8 Hz, 1H), 5.41-5.19 (m, 1H), 4.35-4.22 (m, 6H), 4.12-3.98 (m, 2H), 3.45-3.39 (m, 2H), 3.27-3.12 (m, 3H), 3.05-2.97 (m, 1H), 2.56-2.29 (m, 2H), 2.28-2.08 (m, 6H), 2.01-1.86 (m, 3H), 0.79 (t, J=6.8 Hz, 3H). LCMS (ESI, M+1): m/z=649.1.

### Example 599

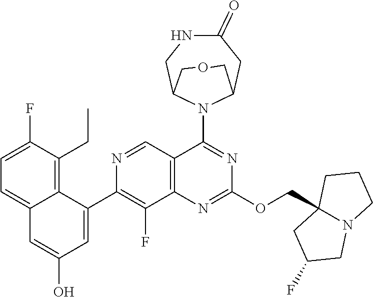 [SMILES: CCc1c(F)ccc2cc(O)cc(-c3ncc4c(N5C6CNC(=O)CC5COC6)nc(OC[C@@]56CCCN5C[C@H](F)C6)nc4c3F)c12]

### 10-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-8-oxa-3,10-diazabicyclo[4.3.1]decan-4-one

The title compound was synthesized using according to the 3-step procedure described for example 585, except for in Step B the mixture was stirred at 100° C. for 1 hour, to produce the desired compound as a yellow solid (0.12 formic acid salt). 1H NMR (400 MHZ, CD₃OD) δ=9.02 (s, 1H), 7.68 (dd, J=5.6, 8.8 Hz, 1H), 7.39-7.17 (m, 2H), 7.04 (dd, J=2.4, 6.0 Hz, 1H), 5.42-5.27 (m, 1H), 5.14-4.91 (m, 4H), 4.45-4.27 (m, 2H), 4.11-3.93 (m, 4H), 3.86-3.74 (m, 2H), 3.20-3.01 (m, 3H), 2.61-1.88 (m, 9H), 0.79 (br t, J=6.8 Hz, 3H); LCMS (ESI, M+1): m/z=649.3.

### Example 600

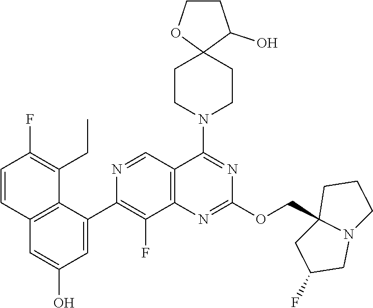 [SMILES: CCc1c(F)ccc2cc(O)cc(-c3ncc4c(N5CCC6(CC5)OCCC6O)nc(OC[C@@]56CCCN5C[C@H](F)C6)nc4c3F)c12]

### 8-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-oxa-8-azaspiro[4.5]decan-4-ol

The title compound was synthesized according to the procedure described for example 538 to produce the desired compound as a white solid (0.41 formic acid salt); ¹H NMR (400 MHZ, METHANOL-d4) δ=0.73-0.86 (m, 3H) 1.75 (br d, J=12.8 Hz, 1H) 1.87-2.06 (m, 5H) 2.09-2.26 (m, 4H) 2.32-2.42 (m, 2H) 2.45-2.54 (m, 1H) 3.14-3.25 (m, 1H) 3.42-3.66 (m, 3H) 3.78-3.95 (m, 3H) 3.99-4.10 (m, 2H) 4.37-4.56 (m, 4H) 5.19-5.63 (m, 1H) 7.06 (d, J=2.4 Hz, 1H) 7.25 (t, J=9.2 Hz, 1H) 7.31 (d, J=2.4 Hz, 1H) 7.68 (dd, J=8.8, 6.0 Hz, 1H) 8.50 (br s, 1H) 9.08 (s, 1H); LCMS (ESI, M+1): m/z=650.3.

### Example 601

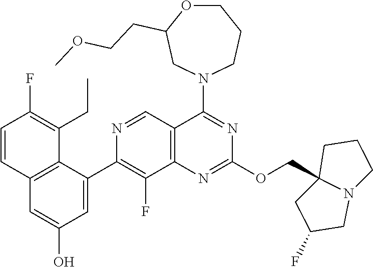 [SMILES: CCc1c(F)ccc2cc(O)cc(-c3ncc4c(N5CCCOC(CCOC)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(2-(2-methoxyethyl)-1,4-oxazepan-4-yl)pyrido[4,3-d]pyrimidin-7-yl)naphthalen-2-ol

The title compound was synthesized according to the procedure described for example 544 to produce the desired compound as a white solid; ¹H NMR (400 MHZ, DMSO-d₆) δ=9.15 (d, J=7.6 Hz, 1H), 7.75-7.58 (m, 1H), 7.36-0.17 (m, 2H), 6.99 (dd, J=2.0, 9.6 Hz, 1H), 5.37-5.17 (m, 1H), 4.46 (br t, J=12.4 Hz, 1H), 4.23-3.95 (m, 6H), 3.65-3.41 (m, 6H), 3.15-2.96 (m, 4H), 2.87-2.78 (m, 1H), 2.33 (br s, 1H), 2.19-1.99 (m, 6H), 1.93-1.64 (m, 6H), 0.73 (td, J=7.2, 12.4 Hz, 3H), LCMS (ESI, M+1): m/z=652.4.

### Example 602

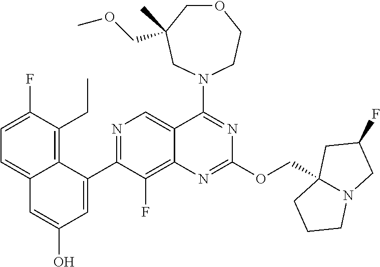 [SMILES: CCc1c(F)ccc2cc(O)cc(-c3ncc4c(N5CCOC[C@@](C)(COC)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((R)-6-(methoxymethyl)-6-methyl-1,4-oxazepan-4-yl)pyrido[4,3-d]pyrimidin-7-yl)naphthalen-2-ol

The title compound was synthesized according to the procedure described for example 538 to produce the desired compound as a yellow solid (1.05 formic acid salt); 1H NMR (400 MHZ, METHANOL-d4) δ=9.28-9.15 (m, 1H), 8.50 (br s, 1H), 7.68 (dd, J=6.0, 8.8 Hz, 1H), 7.31 (d, J=2.4 Hz, 1H), 7.25 (t, J=9.2 Hz, 1H), 7.05 (dd, J=2.4, 6.0 Hz, 1H), 5.51-5.33 (m, 1H), 4.67-4.54 (m, 1H), 4.51-4.36 (m, 3H), 4.34-4.20 (m, 2H), 4.19-4.08 (m, 3H), 3.71 (br dd, J=3.2, 12.8 Hz, 1H), 3.66-3.53 (m, 2H), 3.52-3.42 (m, 2H), 3.19 (br d, J=9.6 Hz, 4H), 2.54-2.32 (m, 3H), 2.27-2.11 (m, 4H), 2.06-1.97 (m, 1H), 1.44-1.24 (m, 1H), 0.98 (d, J=14.8 Hz, 3H), 0.86-0.76 (m, 3H) LCMS [ESI, M+1]: m/z=652.4

### Example 603

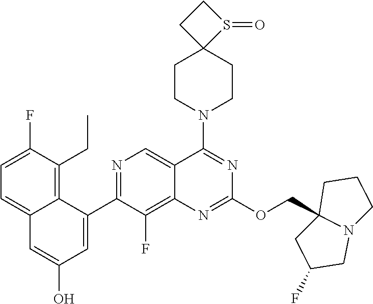 [SMILES: CCc1c(F)ccc2cc(O)cc(-c3ncc4c(N5CCC6(CC5)CCS6=O)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-thia-7-azaspiro[3.5]nonane 1-oxide

The title compound was synthesized according to the procedure described for example 538 to produce the desired compound as a yellow solid. ¹H NMR (400 MHZ, CD₃OD) δ=9.07 (s, 1H), 7.68 (dd, J=6.0, 8.8 Hz, 1H), 7.30 (d, J=2.8 Hz, 1H), 7.25 (t, J=9.6 Hz, 1H), 7.05 (s, 1H), 5.42-5.20 (m, 1H), 4.37-4.20 (m, 4H), 4.14-3.94 (m, 2H), 3.65 (ddd, J=2.8, 8.0, 11.6 Hz, 1H), 3.29-3.09 (m, 4H), 3.06-2.98 (m, 9.2 Hz, 1H), 2.74-2.65 (m, 1H), 2.57-2.44 (m, 2H), 2.39-2.26 (m, 2H), 2.25-2.12 (m, 6H), 2.04-1.88 (m, 3H), 0.80 (br t, J=7.6 Hz, 3H). LCMS (ESI, M+1): m/z=652.3.

### Example 604

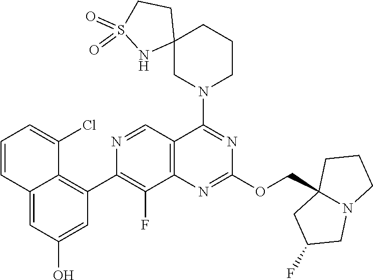 [SMILES: O=S1(=O)CCC2(CCCN(c3nc(OC[C@@]45CCCN4C[C@H](F)C5)nc4c(F)c(-c5cc(O)cc6cccc(Cl)c56)ncc34)C2)N1]

### 7-(7-(8-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide

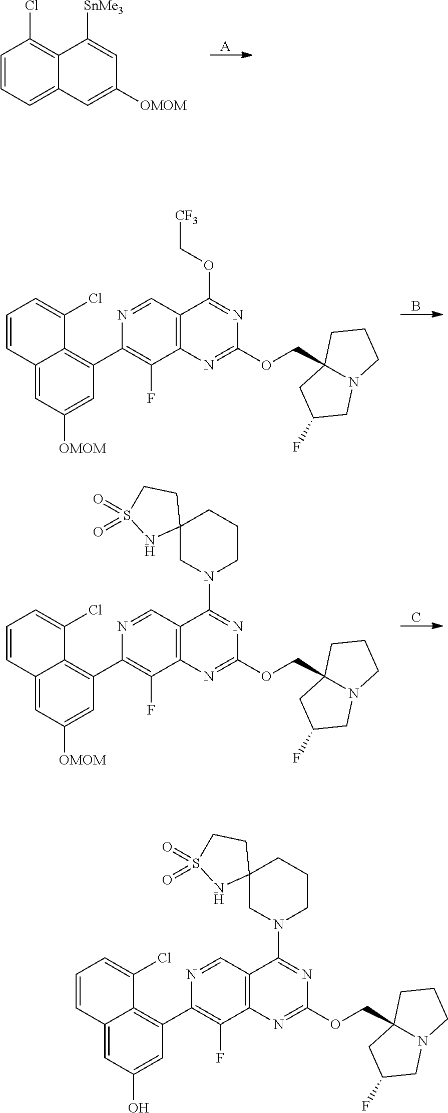

Step A. 7-(8-chloro-3-(methoxymethoxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (500 mg, 1.0 eqniv) and (8-chloro-3-(methoxymethoxy)naphthalen-1-yl)trimethylstannane (528 mg, 1.2 eqniv) in toluene (5 mL) was added AdanBup-Pd-G3 (83.2 mg, 0.10 eqniv) under N₂. The reaction mixture was stirred at 90° C. for 12 hours. The reaction mixture was diluted with water (15 mL) and extracted with ethyl acetate (4×10 mL). The combined organic layers were washed with brine (30 mL), dried over anhydrous sodium sulfate, concentrated and purified by column chromatography [SiO₂, Petroleum ether/Ethyl acetate 5:1 to 1:1] and reversed phase flash chromatography [C18, 0.1% formic acid condition] to afford the title compound (50.0 mg, 7.0% yield) as a yellow oil; LCMS (ESI, M+1): m/z=625.3.

Step B. 7-(7-(8-chloro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide: To a solution of 7-(8-chloro-3-(methoxymethoxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (70.0 mg, 1.0 equiv) and 2-thia-1,7-diazaspiro[4.5]decane 2,2-dioxide (25.6 mg, 1.2 equiv) in dimethyl formamide (0.3 mL) were added DIEA (43.4 mg, 3.0 equiv) and 4 Å molecular sieves (30 mg). The reaction mixture was stirred at 40° C. for 12 hours. The reaction mixture was filtered and purified with reversed phase flash chromatography [C18, 0.1% formic acid condition] to afford the title compound (30.0 mg, 37% yield) as a white solid.

Step C. 7-(7-(8-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide: To a solution of 7-(7-(8-chloro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide (30.0 mg, 1.0 equiv) in acetonitrile (0.5 mL) was added HCl·dioxane (4 M, 1.0 mL). The reaction mixture was stirred at 0° C. for 0.5 hours. The reaction mixture was quenched with saturated aqueous NaHCO₃ (5 mL) at 0° C. and extracted with ethyl acetate (4×5 mL). The combined organic layers were washed with brine (10 mL), dried over anhydrous sodium sulfate, concentrated and purified with prep-HPLC [Waters Xbridge C18 150×50 mm×10 μm; A: water (NH₄HCO₃), B: ACN; B %: 34%-64% over 10 min] to afford the title compound (5.92 mg, 21% yield) as a yellow gum; ¹H NMR (400 MHZ, DMSO-d₆) δ=10.42-10.09 (m, 1H), 9.09 (d, J=4.8 Hz, 1H), 7.85-7.82 (m, 1H), 7.44-7.33 (m, 4H), 7.14-7.12 (m, 1H), 5.43-5.11 (m, 1H), 4.21-3.98 (m, 4H), 3.83-3.71 (m, 1H), 3.67-3.52 (m, 1H), 3.25-3.16 (m, 2H), 3.14 (br s, 2H), 3.02-2.98 (m, 1H), 2.86-2.77 (m, 1H), 2.30-2.10 (m, 3H), 2.07-2.03 (m, 1H), 2.02-1.91 (m, 3H), 1.87-1.74 (m, 5H); LCMS (ESI, M+1): m/z=671.1.

### Example 605

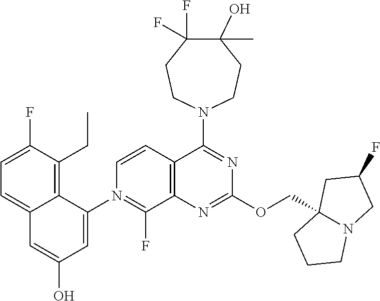

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5,5-difluoro-4-methylazepan-4-ol

The title compound was synthesized according to the procedure described for example 538 to produce the desired compound as a yellow solid. ¹H NMR (400 MHZ, METHANOL-d₄) δ=0.72-0.86 (m, 3H) 1.34 (br s, 3H) 1.93-2.07 (m, 4H) 2.11-2.40 (m, 7H) 2.43-2.54 (m, 1H) 3.03 (br d, J=5.6 Hz, 1H) 3.18-3.28 (m, 3H) 3.89 (br d, J=1.2 Hz, 1H) 4.11-4.41 (m, 6H) 5.38 (br s, 1H) 7.07 (br d, J=9.6 Hz, 1H) 7.21-7.33 (m, 2H) 7.68 (br dd, J=8.4, 6.0 Hz, 1H) 9.17 (d, J=4.0 Hz, 1H). LCMS (ESI, M+1): m/z=658.3.

### Example 606

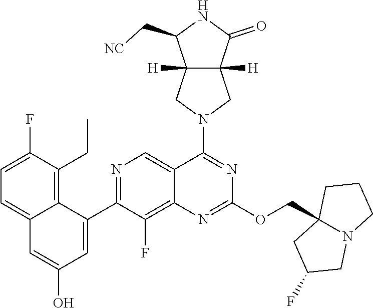 [SMILES: CCc1c(F)ccc2cc(O)cc(-c3ncc4c(N5C[C@@H]6[C@@H](CC#N)NC(=O)[C@@H]6C5)nc(OC[C@@]56CCCN5C[C@H](F)C6)nc4c3F)c12]

### 2-((1R,3aS,6aR)-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-oxooctahydropyrrolo[3,4-c]pyrrol-1-yl) acetonitrile

The title compound was synthesized using Intermediate 13B ((1R,3aS,6aR)-tert-butyl 1-(cyanomethyl)-3-oxohexahydropyrrolo[3,4-c]pyrrole-2(1H)-carboxylate) according to the procedure described for example 566 to produce the desired compound as a white solid (0.5 formic acid salt). ¹H NMR (400 MHZ, METHANOL-d₄) δ=9.34 (d, J=4.8 Hz, 1H), 8.51-8.45 (m, 1H), 7.71 (dd, J=6.0, 9.2 Hz, 1H), 7.33 (d, J=2.4 Hz, 1H), 7.28 (t, J=9.2 Hz, 1H), 7.07 (t, J=3.2 Hz, 1H), 5.56-5.41 (m, 1H), 4.63-4.58 (m, 2H), 4.55-4.48 (m, 2H), 4.38-4.28 (m, 1H), 4.08-3.95 (m, 2H), 3.77-3.56 (m, 4H), 3.30-3.26 (m, 2H), 2.92 (d, J=5.6 Hz, 2H), 2.59-2.43 (m, 3H), 2.36-2.29 (m, 1H), 2.25-2.16 (m, 3H), 2.04 (br s, 1H), 0.81 (q, J=7.2 Hz, 3H); LCMS (ESI, M+1): m/z=658.2.

### Example 607

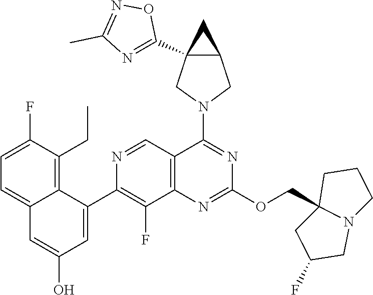 [SMILES: CCc1c(F)ccc2cc(O)cc(-c3ncc4c(N5C[C@@H]6C[C@]6(c6nc(C)no6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5R)-1-(3-methyl-1,2,4-oxadiazol-5-yl)-3-azabicyclo[3.1.0]hexan-3-yl)pyrido[4,3-d]pyrimidin-7-yl)naphthalen-2-ol

The title compound was synthesized according to the procedure described for example 544 to produce the desired compound as a yellow solid. ¹H NMR (400 MHZ, METHANOL-d4) δ=9.27 (d, J=2.4 Hz, 1H), 7.67 (dd, J=6.0, 8.8 Hz, 1H), 7.29 (d, J=2.4 Hz, 1H), 7.24 (t, J=9.2 Hz, 1H), 7.04 (s, 1H), 5.44-5.16 (m, 1H), 4.77-4.46 (m, 3H), 4.39-4.18 (m, 3H), 3.27-3.11 (m, 3H), 3.01 (dt, J=5.6, 9.2 Hz, 1H), 2.64 (br s, 1H), 2.55-2.42 (m, 1H), 2.39-2.35 (m, 3H), 2.31-2.09 (m, 4H), 2.05-1.80 (m, 5H), 1.32-1.28 (m, 1H), 0.79 (q, J=7.2 Hz, 3H). LCMS (ESI, M+1): m/z=658.3.

### Example 608

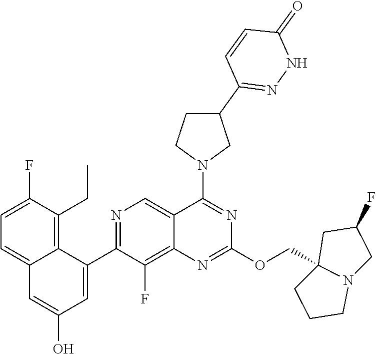 [SMILES: CCc1c(F)ccc2cc(O)cc(-c3ncc4c(N5CCC(c6ccc(=O)[nH]n6)C5)nc(OC[C@@]56CCCN5C[C@H](F)C6)nc4c3F)c12]

### 6-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)pyrrolidin-3-yl)pyridazin-3 (2H)-one

The title compound was synthesized according to the procedure described for example 538 to produce the desired compound as a white solid; 1H NMR (400 MHZ, METHANOL-d4) δ=9.32 (s, 1H), 7.70 (dd, J=6.0, 9.2 Hz, 1H), 7.63 (d, J=9.6 Hz, 1H), 7.33 (d, J=2.8 Hz, 1H), 7.27 (t, J=9.2 Hz, 1H), 7.09-7.00 (m, 2H), 5.57-5.31 (m, 1H), 4.55-4.33 (m, 4H), 3.79-3.56 (m, 2H), 3.55-3.42 (m, 3H), 3.26-3.11 (m, 2H), 2.59-2.46 (m, 2H), 2.43-2.31 (m, 2H), 2.29-2.22 (m, 1H), 2.21-2.09 (m, 3H), 2.03-1.87 (m, 1H), 1.41-1.31 (m, 1H), 0.82 (br t, J=7.6 Hz, 3H). LCMS (ESI, M+1): m/z=658.3.

### Example 609

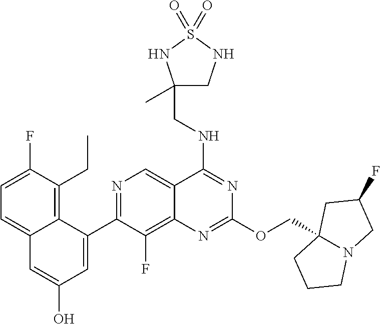 [SMILES: CCc1c(F)ccc2cc(O)cc(-c3ncc4c(NCC5(C)CNS(=O)(=O)N5)nc(OC[C@@]56CCCN5C[C@H](F)C6)nc4c3F)c12]

### 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-3-methyl-1,2,5-thiadiazolidine 1,1-dioxide

The title compound was synthesized using Intermediate 11 according to the procedure described for example 541 to produce the desired compound as a white solid. ¹H NMR (400 MHZ, dimethylsulfoxide-d₆) δ=9.94 (br s, 1H), 9.36 (s, 1H), 8.92 (br d, J=5.2 Hz, 1H), 7.77 (dd, J=2.4, 8.8 Hz, 1H), 7.40-7.30 (m, 2H), 7.22-7.09 (m, 2H), 6.99 (d, J=2.4 Hz, 1H), 5.41-5.17 (m, 1H), 4.21-4.05 (m, 2H), 3.92-3.80 (m, 1H), 3.78-3.66 (m, 1H), 3.49 (dt, J=3.6, 7.6 Hz, 1H), 3.15-3.03 (m, 4H), 2.88-2.81 (m, 1H), 2.39-2.34 (m, 1H), 2.18-1.99 (m, 4H), 1.88-1.71 (m, 3H), 1.34 (s, 3H), 0.72 (br t, J=7.2 Hz, 3H); LCMS (ESI, M+1): m/z=658.2.

### Example 610

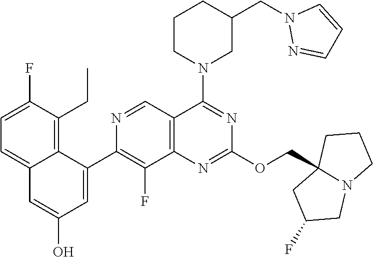 [SMILES: CCc1c(F)ccc2cc(O)cc(-c3ncc4c(N5CCCC(Cn6cccn6)C5)nc(OC[C@@]56CCCN5C[C@H](F)C6)nc4c3F)c12]

### 4-(4-(3-((1H-pyrazol-1-yl)methyl) piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 544 to produce the desired compound as a white solid. ¹H NMR (400 MHZ, CD₃OD) δ=8.93-8.84 (m, 1H), 7.70-7.64 (m, 2H), 7.52 (s, 1H), 7.30 (d, J=2.8 Hz, 1H), 7.25 (t, J=9.6 Hz, 1H), 7.04 (t, J=2.4 Hz, 1H), 6.30-6.28 (m, 1H), 5.41-5.20 (m, 1H), 4.62-4.50 (m, 1H), 4.42-4.31 (m, 1H), 4.27-4.12 (m, 4H), 3.56-3.43 (m, 1H), 3.23-3.16 (m, 2H), 3.03-2.97 (m, 1H), 2.66-2.04 (m, 8H), 2.02-1.90 (m, 4H), 1.86-1.62 (m, 2H), 1.55-1.46 (m, 1H), 0.77 (br t, J=7.6 Hz, 3H); LCMS (ESI, M+1): m/z=658.2.

### Example 611

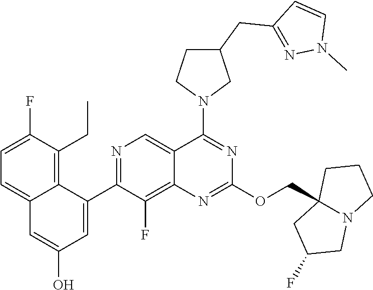 [SMILES: CCc1c(F)ccc2cc(O)cc(-c3ncc4c(N5CCC(Cc6ccn(C)n6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(3-((1-methyl-1H-pyrazol-3-yl)methyl)pyrrolidin-1-yl)pyrido[4,3-d]pyrimidin-7-yl)naphthalen-2-ol

The title compound was synthesized according to the procedure described for example 538 to produce the desired compound as a white solid. ¹H NMR (400 MHZ, METHANOL-d₄) δ=9.22 (br s, 1H), 7.66 (dd, J=6.0, 9.2 Hz, 1H), 7.49 (s, 1H), 7.31-7.20 (m, 2H), 7.04 (dd, J=2.4, 4.8 Hz, 1H), 6.18 (br s, 1H), 5.42-5.21 (m, 1H), 4.35-4.29 (m, 1H), 4.26-4.21 (m, 1H), 4.20-4.06 (m, 2H), 3.89-3.78 (m, 4H), 3.23-3.16 (m, 2H), 3.04-2.96 (m, 1H), 2.92-2.59 (m, 4H), 2.56-2.42 (m, 1H), 2.40-1.82 (m, 10H), 0.79 (br d, J=3.6 Hz, 3H); LCMS (ESI, M+1): m/z=658.3

### Example 612

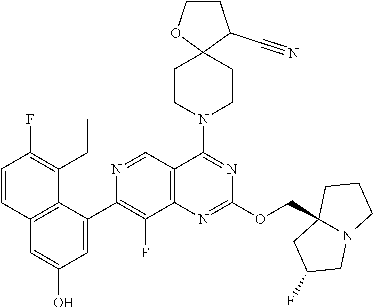 [SMILES: CCc1c(F)ccc2cc(O)cc(-c3ncc4c(N5CCC6(CC5)OCCC6C#N)nc(OC[C@@]56CCCN5C[C@H](F)C6)nc4c3F)c12]

### 8-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-oxa-8-azaspiro[4.5]decane-4-carbonitrile

The title compound was synthesized according to the procedure described for example 538 to produce the desired compound as a white solid. ¹H NMR (400 MHZ, METHANOL-d4) δ=9.08 (s, 1H), 7.68 (br dd, J=6.0, 8.8 Hz, 1H), 7.30 (d, J=2.4 Hz, 1H), 7.25 (brt, J=9.2 Hz, 1H), 7.10-7.03 (m, 1H), 5.48-5.16 (m, 1H), 4.72-4.49 (m, 2H), 4.41-4.22 (m, 2H), 4.18-3.95 (m, 2H), 3.80 (br t, J=10.4 Hz, 2H), 3.31-3.14 (m, 4H), 3.08-2.94 (m, 1H), 2.69-2.44 (m, 2H), 2.41-2.12 (m, 6H), 2.11-1.97 (m, 4H), 1.91 (br d, J=12.4 Hz, 2H), 0.82 (br t, J=7.2 Hz, 3H); LCMS (ESI, M+1): m/z=659.3

### Example 613

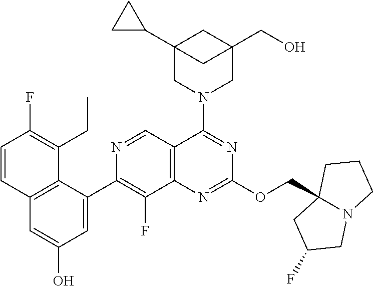 [SMILES: CCc1c(F)ccc2cc(O)cc(-c3ncc4c(N5CC6(CO)CC(C7CC7)(C5)C6)nc(OC[C@@]56CCCN5C[C@H](F)C6)nc4c3F)c12]

### 4-(4-(1-cyclopropyl-5-(hydroxymethyl)-3-azabicyclo[3.1.1]heptan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized according to the procedure described for example 544 to produce the desired compound as a white solid. ¹H NMR (400 MHZ, DMSO-d₆) δ=9.94 (s, 1H), 9.51 (s, 1H), 8.14 (s, 1H), 7.77 (dd, J=6.0, 9.2 Hz, 1H), 7.43-7.24 (m, 2H), 7.02 (d, J=2.4 Hz, 1H), 5.46-5.19 (m, 1H), 4.78 (br s, 1H), 4.36-4.08 (m, 4H), 3.41 (br s, 2H), 3.22-3.10 (m, 2H), 2.98-2.89 (m, 1H), 2.79-2.53 (m, 1H), 2.40-2.32 (m, 1H), 2.24-2.03 (m, 4H), 1.97-1.80 (m, 3H), 1.46 (br d, J=8.4 Hz, 2H), 1.32 (br d, J=7.6 Hz, 2H), 0.95 (br d, J=4.8 Hz, 1H), 0.74 (t, J=7.2 Hz, 3H), 0.47-0.35 (m, 2H), 0.25 (q, J=5.2 Hz, 2H), LCMS (ESI, M+1): m/z=660.4.

### Example 614

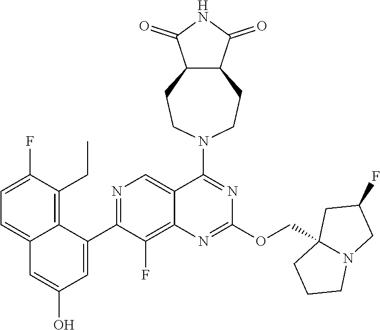 [SMILES: CCc1c(F)ccc2cc(O)cc(-c3ncc4c(N5CC[C@@H]6C(=O)NC(=O)[C@@H]6CC5)nc(OC[C@@]56CCCN5C[C@H](F)C6)nc4c3F)c12]

### (3aR,8aS)-6-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)hexahydropyrrolo[3,4-d]azepine-1,3(2H,3aH)-dione

The title compound was synthesized using Intermediate 12 according to the procedure described for example 538 to produce the desired compound as a white solid; 1H NMR (400 MHZ, DMSO+D2O) δ=9.18-8.98 (m, 1H), 7.75 (dd, J=6.0, 8.8 Hz, 1H), 7.39-7.30 (m, 2H), 7.02 (d, J=2.4 Hz, 1H), 5.49-5.11 (m, 1H), 4.24-4.00 (m, 5H), 3.96-3.80 (m, 1H), 3.21-2.95 (m, 5H), 2.82 (br d, J=6.0 Hz, 1H), 2.40-2.28 (m, 3H), 2.26-2.10 (m, 3H), 2.07-1.90 (m, 3H), 1.88-1.72 (m, 3H), 0.72 (br t, J=7.2 Hz, 3H); LCMS (ESI, M+1): m/z=661.4

### Example 615

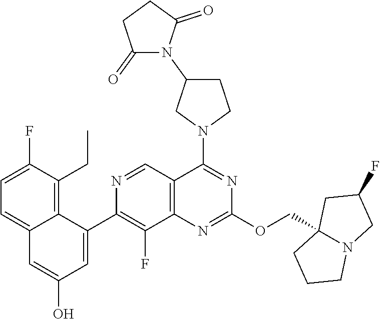 [SMILES: CCc1c(F)ccc2cc(O)cc(-c3ncc4c(N5CCC(N6C(=O)CCC6=O)C5)nc(OC[C@@]56CCCN5C[C@H](F)C6)nc4c3F)c12]

### 1′-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-[1,3′-bipyrrolidine]-2,5-dione

The title compound was synthesized according to the procedure described for example 544 to produce the desired compound as a white solid. ¹H NMR (400 MHZ, DMSO-d6) δ ppm 0.66-0.79 (m, 3H) 1.70-1.90 (m, 3H) 1.95-2.18 (m, 4H) 2.20-2.40 (m, 3H) 2.60-2.68 (m, 5H) 2.79-2.87 (m, 1H) 2.99-3.14 (m, 4H) 4.00-4.19 (m, 4H) 4.79-4.91 (m, 1H) 5.18-5.37 (m, 1H) 7.01 (s, 1H) 7.31-7.39 (m, 2H) 7.76 (dd, J=8.8, 6.0 Hz, 1H) 8.15 (s, 1H) 9.24 (br d, J=8.0 Hz, 1H); LCMS (ESI, M+1): m/z=661.6.

### Example 616

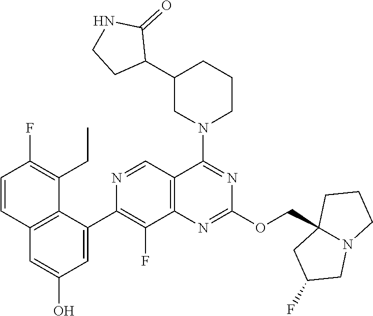 [SMILES: CCc1c(F)ccc2cc(O)cc(-c3ncc4c(N5CCCC(C6CCNC6=O)C5)nc(OC[C@@]56CCCN5C[C@H](F)C6)nc4c3F)c12]

### 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one

The title compound was synthesized according to the procedure described for example 538 to produce the desired compound as a white solid. 1H NMR (400 MHZ, METHANOL-d4) δ=9.21-9.01 (m, 1H), 7.68 (dd, J=6.0, 9.2 Hz, 1H), 7.30 (d, J=2.4 Hz, 1H), 7.25 (t, J=9.2 Hz, 1H), 7.09-7.03 (m, 1H), 5.48-5.19 (m, 1H), 5.11 (br d, J=13.2 Hz, 1H), 4.69 (br d, J=10.0 Hz, 1H), 4.47-4.18 (m, 2H), 3.41-3.35 (m, 2H), 3.31 (br s, 1H), 3.28-3.13 (m, 3H), 3.08-2.95 (m, 1H), 2.60-2.39 (m, 2H), 2.36-2.14 (m, 6H), 2.10-1.53 (m, 9H), 0.88-0.73 (m, 3H); LCMS (ESI, M+1): m/z=661.3.

### Example 617

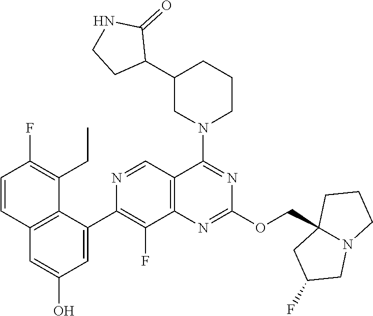 [SMILES: CCc1c(F)ccc2cc(O)cc(-c3ncc4c(N5CCCC(C6CCNC6=O)C5)nc(OC[C@@]56CCCN5C[C@H](F)C6)nc4c3F)c12]

Peak 1 stereoisomer of 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one

### Example 618

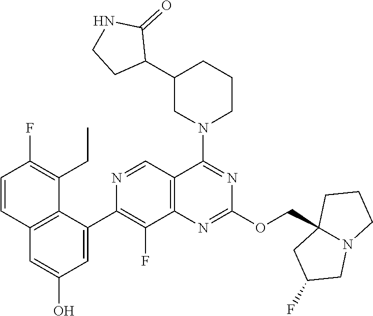 [SMILES: CCc1c(F)ccc2cc(O)cc(-c3ncc4c(N5CCCC(C6CCNC6=O)C5)nc(OC[C@@]56CCCN5C[C@H](F)C6)nc4c3F)c12]

Peak 2 stereoisomer of 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one

### Example 619

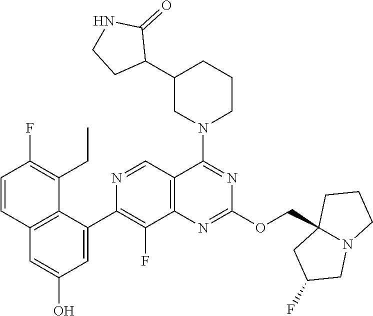 [SMILES: CCc1c(F)ccc2cc(O)cc(-c3ncc4c(N5CCCC(C6CCNC6=O)C5)nc(OC[C@@]56CCCN5C[C@H](F)C6)nc4c3F)c12]

Peak 3 stereoisomer of 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one

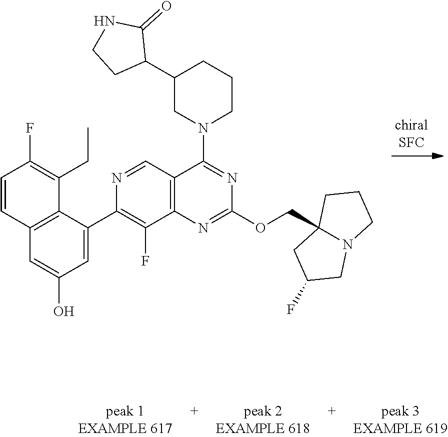

3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one (30 mg, 1.0 equiv) was purified by chiral SFC (column: REGIS (S,S) WHELK-O1 (250 mm×25 mm, 10 um); mobile phase: [0.1% NH₃H₂O MEOH]; B %: 40%-40%, 5.0 min) and lyophilized to afford peak 1, Example 617 (6 mg, 9.08 μmol, 20% yield) as a white solid; 1H NMR (400 MHz, METHANOL-d4) δ=9.09 (d, J=1.2 Hz, 1H), 7.69 (dd, J=6.0, 8.8 Hz, 1H), 7.32 (d, J=2.8 Hz, 1H), 7.26 (t, J=9.2 Hz, 1H), 7.12-7.03 (m, 1H), 5.50-5.22 (m, 1H), 4.79-4.57 (m, 2H), 4.41 (d, J=10.8 Hz, 1H), 4.26 (d, J=10.4 Hz, 1H), 3.32-3.14 (m, 3H), 3.13-2.96 (m, 1H), 2.61-2.43 (m, 2H), 2.40 (br d, J=1.6 Hz, 8H), 2.10-1.82 (m, 7H), 1.79-1.53 (m, 3H), 1.43-1.36 (m, 1H), 0.82 (q, J=6.8 Hz, 3H); LCMS (ESI, M+1): m/z=661.4, peak 2, Example 618 (6 mg, 20.0% yield) as a white solid; ¹H NMR (400 MHZ, METHANOL-d4) δ=9.11 (s, 1H), 7.67 (dd, J=6.0, 8.8 Hz, 1H), 7.30 (d, J=2.8 Hz, 1H), 7.25 (t, J=9.2 Hz, 1H), 7.06 (dd, J=2.4, 4.8 Hz, 1H), 5.51-5.26 (m, 1H), 5.15 (br d, J=13.6 Hz, 1H), 4.68 (br d, J=12.4 Hz, 1H), 4.38 (d, J=3.2 Hz, 2H), 3.56-3.38 (m, 3H), 3.16-3.03 (m, 1H), 2.54-2.42 (m, 2H), 2.39-2.05 (m, 8H), 2.02-1.86 (m, 4H), 1.84-1.50 (m, 3H), 1.34-1.27 (m, 2H), 1.21-1.12 (m, 1H), 0.84-0.73 (m, 3H); LCMS (ESI, M+1): m/z=661.4, and peak 3, Example 619 (5 mg, 50% yield) as a white solid; ¹H NMR (400 MHZ, METHANOL-d4) δ=9.11 (s, 1H), 7.69 (dd, J=6.0, 9.2 Hz, 1H), 7.31 (d, J=2.4 Hz, 1H), 7.26 (t, J=9.6 Hz, 1H), 7.09-7.04 (m, 1H), 5.41-5.23 (m, 1H), 5.11 (br d, J=11.6 Hz, 1H), 4.79-4.51 (m, 2H), 4.41-4.21 (m, 2H), 3.29-3.15 (m, 3H), 3.09-2.98 (m, 1H), 2.57-2.42 (m, 2H), 2.39-2.09 (m, 8H), 2.08-1.88 (m, 7H), 1.86-1.53 (m, 3H), 1.28 (s, 1H), 0.87-0.77 (m, 3H); LCMS (ESI, M+1): m/z=661.4.

### Example 620

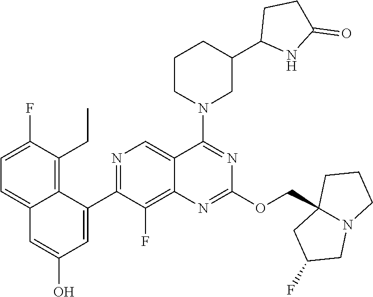 [SMILES: CCc1c(F)ccc2cc(O)cc(-c3ncc4c(N5CCCC(C6CCC(=O)N6)C5)nc(OC[C@@]56CCCN5C[C@H](F)C6)nc4c3F)c12]

### 5-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)pyrrolidin-2-one

The title compound was synthesized according to the procedure described for example 538 to produce the desired compound as a white solid (0.24 formic acid salt). ¹H NMR (400 MHZ, DMSO-d₆) δ=9.08 (d, J=14.2 Hz, 1H), 8.13-7.98 (m, 1H), 7.77 (dd, J=6.0, 8.8 Hz, 1H), 7.44-7.30 (m, 2H), 7.03 (s, 1H), 5.45-5.09 (m, 1H), 4.70-4.46 (m, 2H), 4.07 (s, 2H), 3.16-3.05 (m, 3H), 3.02 (br s, 2H), 2.83 (br d, J=6.0 Hz, 2H), 2.23-1.94 (m, 9H), 1.93-1.58 (m, 10H), 1.48-1.14 (m, 2H), 0.73 (br d, J=7.2 Hz, 3H); LCMS (ESI, M+1): m/z=661.3

### Example 621

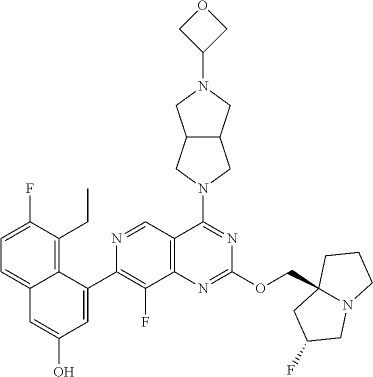 [SMILES: CCc1c(F)ccc2cc(O)cc(-c3ncc4c(N5CC6CN(C7COC7)CC6C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(5-(oxetan-3-yl)hexahydropyrrolo[3,4-c]pyrrol-2(1H)-yl)pyrido[4,3-d]pyrimidin-7-yl)naphthalen-2-ol

The title compound was synthesized according to the procedure described for example 544 to produce the desired compound as a yellow solid; ¹H NMR (400 MHZ, CHLOROFORM-d) δ=9.26 (s, 1H), 7.68-7.61 (m, 1H), 7.27 (br s, 1H), 7.23 (br t, J=9.6 Hz, 1H), 7.04 (d, J=2.8 Hz, 1H), 5.43-5.19 (m, 1H), 4.73 (t, J=6.4 Hz, 2H), 4.66-4.55 (m, 2H), 4.43-4.22 (m, 4H), 4.14-4.00 (m, 2H), 3.71 (quin, J=6.0 Hz, 1H), 3.25-3.12 (m, 4H), 3.08-2.93 (m, 1H), 2.80-2.63 (m, 4H), 2.57-2.43 (m, 1H), 2.41-2.09 (m, 4H), 2.04-1.84 (m, 3H), 0.80 (br t, J=7.2 Hz, 3H); LCMS (ESI, M+1): m/z=661.3.

### Example 622

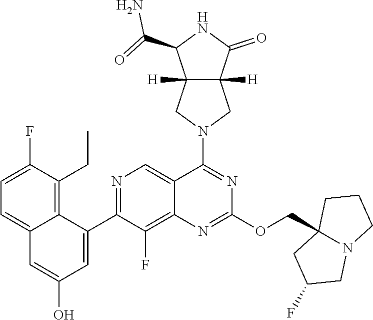 [SMILES: CCc1c(F)ccc2cc(O)cc(-c3ncc4c(N5C[C@@H]6[C@@H](C(N)=O)NC(=O)[C@@H]6C5)nc(OC[C@@]56CCCN5C[C@H](F)C6)nc4c3F)c12]

### (3aS,6aR)-5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxooctahydropyrrolo[3,4-c]pyrrole-1-carboxamide

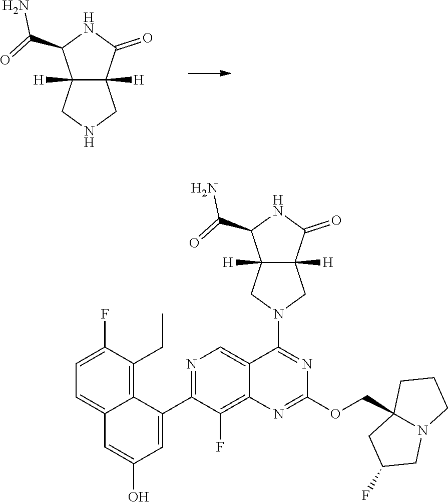 [SMILES: NC(=O)[C@H]1NC(=O)[C@@H]2CNC[C@H]12 ; CCc1c(F)ccc2cc(O)cc(-c3ncc4c(N5C[C@@H]6[C@@H](C(N)=O)NC(=O)[C@@H]6C5)nc(OC[C@@]56CCCN5C[C@H](F)C6)nc4c3F)c12]

A mixture of (1S,3aS,6aR)-3-oxooctahydropyrrolo[3,4-c]pyrrole-1-carboxamide, Intermediate 13A (13.0 mg, 1.0 equiv), 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (54.6 mg, 1.2 equiv), K₃PO₄ (48.9 mg, 230.5 μmol, 3.0 equiv) in DMF (0.5 mL) was degassed and purged with N₂ for 3 times, and then the mixture was stirred at 40° C. for 12 hours under N₂ atmosphere. The mixture was filtered and purified by prep-HPLC [C18, 0.1% formic acid condition] and lyophilized to afford the title compound (10 mg, 19.5% yield) as a white solid. ¹H NMR (400 MHZ, DMSO-d6) δ=9.32 (d, J=5.6 Hz, 1H), 8.20 (s, 2H), 7.77 (dd, J=6.0, 9.2 Hz, 1H), 7.66-7.53 (m, 1H), 7.39-7.32 (m, 2H), 7.27 (br s, 1H), 7.03 (t, J=2.8 Hz, 1H), 5.39-5.17 (m, 1H), 4.52-4.39 (m, 1H), 4.17 (td, J=4.8, 10.0 Hz, 3H), 4.11-4.05 (m, 1H), 4.02 (s, 1H), 3.95-3.85 (m, 1H), 3.23-3.05 (m, 5H), 3.03 (s, 1H), 2.88-2.80 (m, 1H), 2.40-2.28 (m, 1H), 2.18-1.97 (m, 4H), 1.92-1.73 (m, 3H), 0.83-0.63 (m, 3H). ¹⁹F NMR (376 MHz, DMSO-d₆) δ=−119.67 (br d, J=14.0 Hz, 1F), −134.92-−141.96 (m, 1F), −169.38-−176.42 (m, 1F). LCMS (ESI, M+1): m/z=662.5.

### Example 623

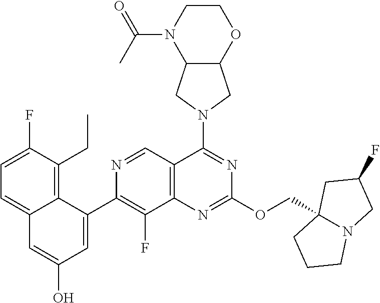 [SMILES: CCc1c(F)ccc2cc(O)cc(-c3ncc4c(N5CC6OCCN(C(C)=O)C6C5)nc(OC[C@@]56CCCN5C[C@H](F)C6)nc4c3F)c12]

### 1-(6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)hexahydropyrrolo[3,4-b][1,4]oxazin-4(4aH)-yl) ethan-1-one

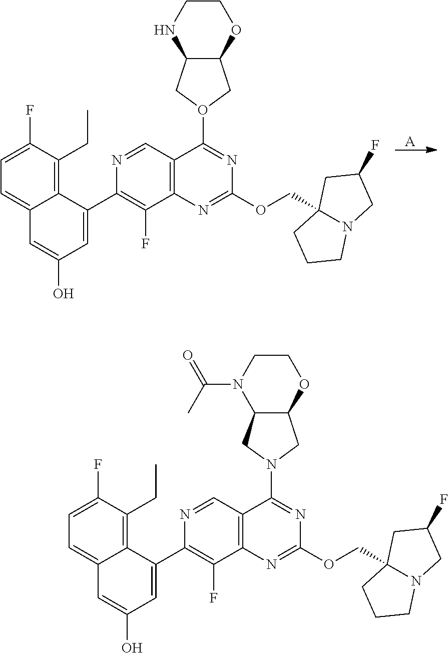

To a solution of acetyl chloride (1.70 mg, 1.2 equiv) in a mixed solvent of DCM (0.5 mL) and MeOH (0.1 mL) were added TEA (5.50 mg, 3.0 equiv) and 5-ethyl-6-fluoro-4-(8-fluoro-2-(((2R,7aS)-2-fluorotetrahydro-1H-pyrrolizin-7a(5H)-yl)methoxy)-4-(hexahydropyrrolo[3,4-b][1,4]oxazin-6 (2H)-yl)pyrido[4,3-d]pyrimidin-7-yl)naphthalen-2-ol (12.0 mg, 1.0 equiv, HCl). The mixture was stirred at 0° C. for 0.5 hour. The reaction mixture was diluted with water (5 mL) and extracted with EtOAc (3 mL×3). The combined organic layers were washed with brine (8 mL), dried over Na₂SO₄, filtered and concentrated. The residue was purified with prep-HPLC [C18, 0.1% formic acid condition] to afford the title compound (2.00 mg, 16% yield) as a white solid. ¹H NMR (400 MHZ, methanol-d₄) δ=9.27 (br d, J=6.4 Hz, 1H), 8.50 (s, 1H), 7.68 (dd, J=5.6, 8.8 Hz, 1H), 7.31 (d, J=2.8 Hz, 1H), 7.25 (t, J=9.6 Hz, 1H), 7.09-6.99 (m, 1H), 5.52-5.31 (m, 1H), 5.16-5.05 (m, 1H), 4.53-4.44 (m, 1H), 4.44-4.37 (m, 1H), 4.33-4.18 (m, 3H), 4.13 (ddd, J=6.0, 9.2, 13.6 Hz, 1H), 4.05-3.98 (m, 1H), 3.76-3.66 (m, 1H), 3.65-3.54 (m, 2H), 3.52-3.34 (m, 4H), 3.23-3.12 (m, 1H), 2.54-2.31 (m, 3H), 2.24 (br s, 2H), 2.21-2.15 (m, 3H), 2.14-2.07 (m, 2H), 2.06-1.92 (m, 1H), 0.84-0.74 (m, 3H); ¹⁹F NMR (376 MHz, methanol-d₄) δ=−121, −139, −174; LCMS (ESI, M+1): m/z=663.3.

### Example 624

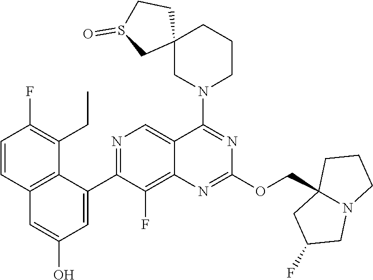 [SMILES: CCc1c(F)ccc2cc(O)cc(-c3ncc4c(N5CCC[C@@]6(CC[S@](=O)C6)C5)nc(OC[C@@]56CCCN5C[C@H](F)C6)nc4c3F)c12]

### (2S,5R)-7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-7-azaspiro[4.5]decane 2-oxide (Stereochemistry was Arbitrarily Assigned)

### Example 625

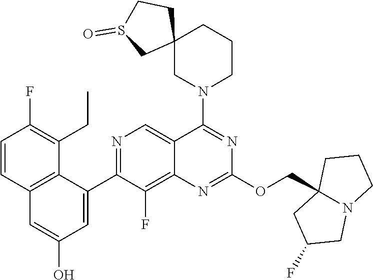 [SMILES: CCc1c(F)ccc2cc(O)cc(-c3ncc4c(N5CCC[C@]6(CC[S@](=O)C6)C5)nc(OC[C@@]56CCCN5C[C@H](F)C6)nc4c3F)c12]

### (2S,5S)-7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-7-azaspiro[4.5]decane 2-oxide (Stereochemistry was Arbitrarily Assigned)

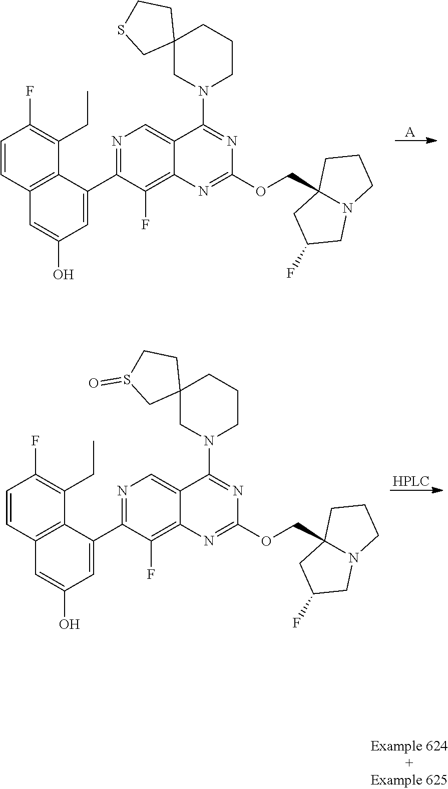

Step A: 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-7-azaspiro[4.5]decane 2-oxide: To a solution of NaIO₄ (69.1 mg, 1.0 equiv) in H₂O (1.5 mL) was added a solution of 5-ethyl-6-fluoro-4-(8-fluoro-2-(((2R,7aS)-2-fluorohexahydro-1H-pyrrolizin-7a-yl)methoxy)-4-(2-thia-7-azaspiro[4.5]decan-7-yl)pyrido[4,3-d]pyrimidin-7-yl)naphthalen-2-ol (0.20 g, 1.0 equiv) in MeOH (1.2 mL) and dioxane (0.9 mL) at 0° C. The reaction was stirred at 15° C. for 12 hours. The mixture was diluted with H₂O (10 mL) and extracted with EtOAc (3×30 mL). The combined organic layer was washed with brine (3×50 mL), dried over anhydrous sodium sulfate, concentrated and purified by prep-HPLC; column: Unisil 3-100 C18 Ultra 150×50 mm×3 μm, mobile phase: A: water (10 mM formic acid), B: ACN, B %: 13%-43% over 7 min to afford peak 1, Example 625 (24.4 mg, 10% yield). White solid; ¹HNMR (400 MHZ, MeOH-d4) δ 9.11 (s, 1H), 7.71 (dd, J=6.0, 9.2 Hz, 1H), 7.33 (d, J=2.4 Hz, 1H), 7.31-7.25 (m, 1H), 7.08 (d, J=2.4 Hz, 1H), 5.51-5.27 (m, 1H), 4.46-4.25 (m, 2H), 4.21-4.05 (m, 2H), 4.01-3.88 (m, 2H), 3.52-3.36 (m, 4H), 3.19-3.10 (m, 2H), 2.78 (br d, J=14.4 Hz, 1H), 2.56-2.47 (m, 2H), 2.45-2.29 (m, 3H), 2.25-2.16 (m, 2H), 2.14-2.07 (m, 4H), 2.03-1.83 (m, 4H), 0.83 (t, J=7.2 Hz, 3H); LCMS (ESI, M+1): m/z=666.4. and peak 2, Example 624 (42.2 mg, 19% yield) as a white solid; ¹HNMR (400 MHZ, MeOH-d₄) δ 9.11 (s, 1H), 7.68 (dd, J=5.6, 9.2 Hz, 1H), 7.31 (d, J=2.4 Hz, 1H), 7.25 (t, J=9.2 Hz, 1H), 7.06 (dd, J=2.8, 4.4 Hz, 1H), 5.61-5.22 (m, 1H), 4.62-4.46 (m, 4H), 4.22-3.99 (m, 2H), 3.67-3.36 (m, 3H), 3.26-3.03 (m, 4H), 2.92-2.79 (m, 1H), 2.71-2.56 (m, 1H), 2.54-2.24 (m, 4H), 2.22-1.99 (m, 5H), 1.99-1.77 (m, 4H), 0.80 (br t, J=7.2 Hz, 3H); LCMS (ESI, M+1): m/z=666.3.

### Example 626

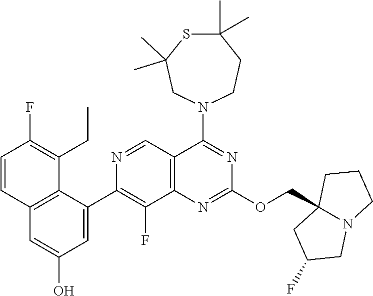 [SMILES: CCc1c(F)ccc2cc(O)cc(-c3ncc4c(N5CCC(C)(C)SC(C)(C)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(2,2,7,7-tetramethyl-1,4-thiazepan-4-yl)pyrido[4,3-d]pyrimidin-7-yl)naphthalen-2-ol

The title compound was synthesized according to the procedure described for example 538 to produce the desired compound as a white solid; ¹H NMR (400 MHZ, METHANOL-d4) δ=9.12 (s, 1H), 7.68 (dd, J=5.6, 9.2 Hz, 1H), 7.31 (d, J=2.8 Hz, 1H), 7.26 (t, J=9.6 Hz, 1H), 7.08 (d, J=2.4 Hz, 1H), 5.47-5.21 (m, 1H), 4.65-4.48 (m, 2H), 4.43-4.33 (m, 2H), 4.32-4.18 (m, 3H), 3.50-3.33 (m, 2H), 3.15-3.03 (m, 1H), 2.58-2.31 (m, 4H), 2.30-2.12 (m, 3H), 2.10-1.84 (m, 3H), 1.50-1.40 (m, 12H), 0.81 (br t, J=6.8 Hz, 3H); LCMS [ESI, M+1]: m/z=666.4

### Example 627

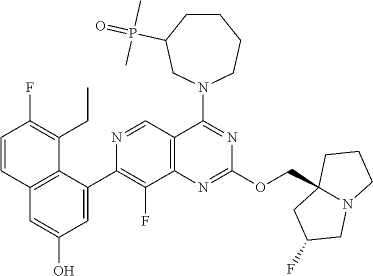 [SMILES: CCc1c(F)ccc2cc(O)cc(-c3ncc4c(N5CCCCC(P(C)(C)=O)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) azepan-3-yl)dimethylphosphine oxide

The title compound was synthesized using intermediate 14 according to the procedure described for example 544 to produce the desired compound as a white solid. ¹H NMR (400 MHZ, METHANOL-d₄) δ=9.39 (br s, 1H), 7.74 (dd, J=6.0, 8.8 Hz, 1H), 7.40 (br s, 1H), 7.31 (t, J=9.2 Hz, 1H), 7.21-7.07 (m, 1H), 5.72-5.52 (m, 1H), 5.51-5.28 (m, 1H), 5.24-5.05 (m, 1H), 4.76-4.56 (m, 1H), 4.48-4.17 (m, 2H), 4.09-3.92 (m, 2H), 3.91-3.77 (m, 2H), 3.45 (br d, J=7.6 Hz, 1H), 2.70 (br s, 5H), 2.40-2.23 (m, 5H), 2.17-1.98 (m, 3H), 1.73 (br d, J=12.4 Hz, 3H), 1.61 (br d, J=12.0 Hz, 3H), 1.58-1.40 (m, 2H), 0.93-0.83 (m, 3H); LCMS (ESI, M+1): m/z=668.3.

### Example 628

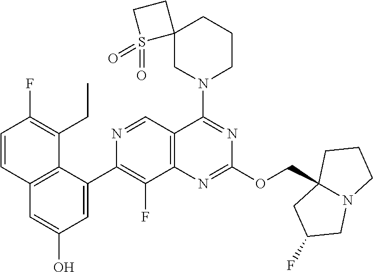 [SMILES: CCc1c(F)ccc2cc(O)cc(-c3ncc4c(N5CCCC6(CCS6(=O)=O)C5)nc(OC[C@@]56CCCN5C[C@H](F)C6)nc4c3F)c12]

### 6-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-thia-6-azaspiro[3.5]nonane 1,1-dioxide

The title compound was synthesized according to the procedure described for example 538 to produce the desired compound as a white solid; ¹H NMR (400 MHZ, METHANOL-d4) δ=9.13 (br d, J=4.4 Hz, 1H), 7.75-7.62 (m, 1H), 7.31 (d, J=2.4 Hz, 1H), 7.25 (dt, J=2.8, 9.6 Hz, 1H), 7.12-7.01 (m, 1H), 5.54-5.35 (m, 1H), 5.24-4.98 (m, 2H), 4.59 (s, 1H), 4.56-4.38 (m, 2H), 4.07-3.95 (m, 3H), 3.55-3.40 (m, 4H), 3.27-3.11 (m, 1H), 2.76-2.62 (m, 1H), 2.54-2.34 (m, 3H), 2.31-2.23 (m, 1H), 2.16-2.08 (m, 4H), 2.06-1.90 (m, 4H), 0.84-0.75 (m, 3H); LCMS (ESI, M+1): m/z=668.3.

### Example 629

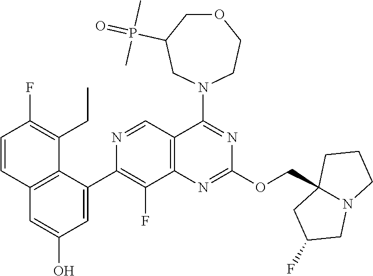 [SMILES: CCc1c(F)ccc2cc(O)cc(-c3ncc4c(N5CCOCC(P(C)(C)=O)C5)nc(OC[C@@]56CCCN5C[C@H](F)C6)nc4c3F)c12]

### (4-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1,4-oxazepan-6-yl)dimethylphosphine oxide

The title compound was synthesized using Intermediate 4 according to the procedure described for example 541 to produce the desired compound as a yellow solid; ¹H NMR (400 MHZ, DMSO-d₆) δ=9.15 (d, J=2.0 Hz, 1H), 7.77 (dd, J=6.0, 8.8 Hz, 1H), ¹H NMR (400 MHz, DMSO-d₆) δ=9.21 (d, J=2.4 Hz, 1H), 7.76 (dd, J=6.0, 8.8 Hz, 1H), 7.39-7.31 (m, 2H), 7.02 (dd, J=2.0, 11.6 Hz, 1H), 5.36-5.18 (m, 1H), 4.78-4.62 (m, 1H), 4.19-4.02 (m, 6H), 3.88-3.75 (m, 2H), 3.15-2.97 (m, 4H), 2.83 (br d, J=6.8 Hz, 2H), 2.14-1.99 (m, 4H), 1.90-1.71 (m, 4H), 1.60-1.51 (m, 6H), 0.76-0.68 (m, 3H), LCMS (ESI, M+1): m/z=670.1.

### Example 630

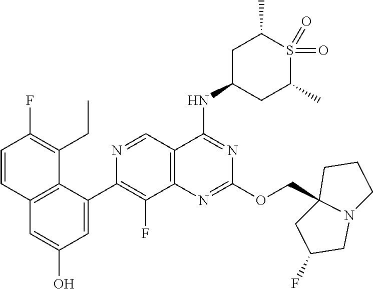 [SMILES: CCc1c(F)ccc2cc(O)cc(-c3ncc4c(N[C@@H]5C[C@@H](C)S(=O)(=O)[C@@H](C)C5)nc(OC[C@@]56CCCN5C[C@H](F)C6)nc4c3F)c12]

### (2R,4s,6S)-4-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)-2,6-dimethyltetrahydro-2H-thiopyran 1,1-dioxide

The title compound was synthesized according to the procedure described for example 538 to produce the desired compound as a white solid; ¹H NMR (400 MHZ, methanol-d₄) δ=9.41 (s, 1H), 7.68 (dd, J=6.0, 9.2 Hz, 1H), 7.31 (d, J=2.4 Hz, 1H), 7.25 (t, J=9.2 Hz, 1H), 7.03 (d, J=2.4 Hz, 1H), 5.40-5.22 (m, 1H), 4.72 (br d, J=2.8 Hz, 1H), 4.38-4.23 (m, 2H), 3.75-3.54 (m, 2H), 3.24-3.17 (m, 2H), 3.06-2.97 (m, 1H), 2.60-2.42 (m, 3H), 2.28-2.10 (m, 6H), 2.06-1.84 (m, 4H), 1.35 (dd, J=4.8, 6.5 Hz, 6H), 0.79 (t, J=7.2 Hz, 3H); LCMS (ESI, M+1): m/z=670.3.

### Example 631

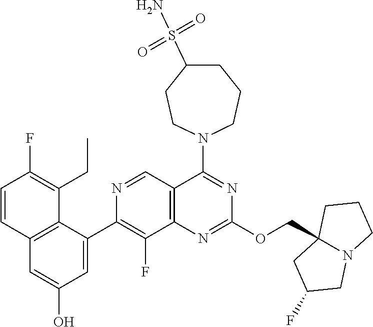 [SMILES: CCc1c(F)ccc2cc(O)cc(-c3ncc4c(N5CCCC(S(N)(=O)=O)CC5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azepane-4-sulfonamide

The title compound was synthesized according to the procedure described for example 544 to produce the desired compound as a white solid (0.38 formic acid salt); ¹H NMR (400 MHZ, DMSO-d₆) δ=0.73 (dt, J=10.8, 7.2 Hz, 3H) 1.54-1.67 (m, 1H) 1.73-1.90 (m, 3H) 1.97-2.21 (m, 6H) 2.26-2.43 (m, 2H) 2.67 (br d, J=1.6 Hz, 1H) 2.78-2.94 (m, 1H) 3.02-3.10 (m, 2H) 3.10-3.16 (m, 2H) 3.90 (br s, 1H) 4.07-4.34 (m, 4H) 5.23 (br s, 1H) 5.36 (br s, 1H) 6.80 (s, 1H) 6.95-7.05 (m, 1H) 7.27-7.41 (m, 2H) 7.76 (dd, J=8.8, 6.0 Hz, 1H) 8.14 (s, 1H) 8.97 (s, 1H) 9.16 (s, 1H) 9.94 (br s, 1H); LCMS (ESI, M+1): m/z=671.3.

### Example 632

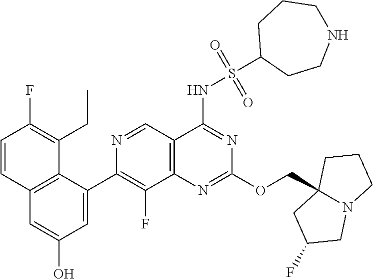 [SMILES: CCc1c(F)ccc2cc(O)cc(-c3ncc4c(NS(=O)(=O)C5CCCNCC5)nc(OC[C@@]56CCCN5C[C@H](F)C6)nc4c3F)c12]

### N-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azepane-4-sulfonamide

The title compound was synthesized according to the procedure described for example 538 to produce the desired compound as a a white solid; (0.49 formic acid salt) ¹H NMR (400 MHZ, DMSO-d₆) δ=0.71 (t, J=7.2 Hz, 3H) 1.71-1.94 (m, 4H) 2.02-2.26 (m, 7H) 2.30-2.41 (m, 3H) 2.58-2.76 (m, 1H) 2.85-2.95 (m, 1H) 3.12 (br s, 3H) 3.17-3.26 (m, 3H) 3.61 (br d, J=4.4 Hz, 1H) 4.06-4.19 (m, 2H) 5.22-5.41 (m, 1H) 6.97 (s, 1H) 7.28-7.36 (m, 2H) 7.75 (dd, J=8.8, 6.0 Hz, 1H) 8.13-8.16 (m, 1H) 8.14 (s, 1H) 9.04 (s, 1H) 9.88 (br s, 1H); LCMS (ESI, M+1): m/z=671.3.

### Example 633

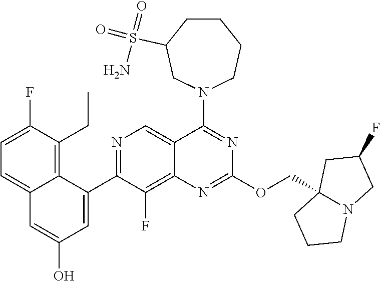 [SMILES: CCc1c(F)ccc2cc(O)cc(-c3ncc4c(N5CCCCC(S(N)(=O)=O)C5)nc(OC[C@@]56CCCN5C[C@H](F)C6)nc4c3F)c12]

### 1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)azepane-3-sulfonamide

The title compound was synthesized according to the procedure described for example 538 to produce the desired compound as a a white solid (0.49 formic acid salt); ¹H NMR (400 MHZ, METHANOL-d₄) δ=9.18-9.15 (m, 1H), 9.16 (s, 1H), 8.52 (s, 1H), 7.67 (dd, J=6.0, 9.2 Hz, 1H), 7.30 (d, J=2.8 Hz, 1H), 7.24 (t, J=9.2 Hz, 1H), 7.05 (d, J=2.4 Hz, 1H), 5.41-5.41 (m, 1H), 5.55-5.39 (m, 1H), 4.73-4.66 (m, 1H), 4.65-4.58 (m, 1H), 4.04-3.83 (m, 3H), 3.74-3.57 (m, 3H), 3.24-3.12 (m, 1H), 2.60-2.39 (m, 5H), 2.32-2.19 (m, 2H), 2.18-2.05 (m, 3H), 2.04-1.86 (m, 3H), 1.81-1.64 (m, 1H), 0.78 (br t, J=6.8 Hz, 3H); LCMS (ESI, M+1): m/z=671.4.

### Example 634

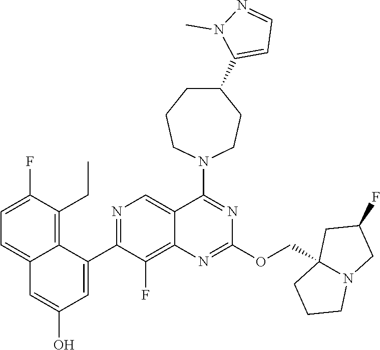 [SMILES: CCc1c(F)ccc2cc(O)cc(-c3ncc4c(N5CCC[C@H](c6ccnn6C)CC5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((S)-4-(1-methyl-1H-pyrazol-5-yl) azepan-1-yl)pyrido[4,3-d]pyrimidin-7-yl)naphthalen-2-ol

The title compound was synthesized according to the procedure described for example 544 to produce the desired compound as a white solid; ¹H NMR (400 MHZ, DMSO-d₆) δ=9.17 (br s, 1H), 8.19 (s, 1H), 7.76 (br dd, J=6.0, 8.5 Hz, 1H), 7.45-7.17 (m, 3H), 7.01 (br d, J=11.2 Hz, 1H), 6.03 (br d, J=14.8 Hz, 1H), 5.45-5.13 (m, 1H), 4.34-3.89 (m, 5H), 3.76 (s, 3H), 3.15-2.97 (m, 4H), 2.87-2.64 (m, 2H), 2.28-1.96 (m, 8H), 1.91-1.44 (m, 5H), 0.88-0.61 (m, 3H), LCMS (ESI, M+1): m/z=672.4.

### Example 635

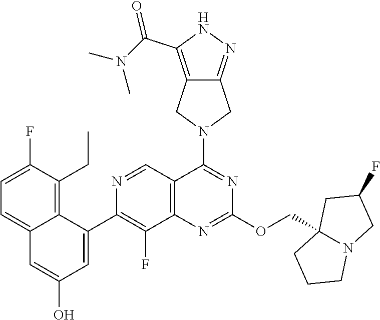 [SMILES: CCc1c(F)ccc2cc(O)cc(-c3ncc4c(N5Cc6n[nH]c(C(=O)N(C)C)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-2,4,5,6-tetrahydropyrrolo[3,4-c]pyrazole-3-carboxamide

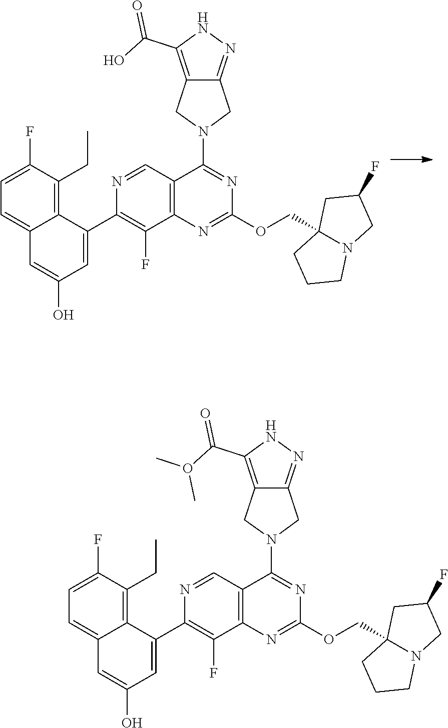

To a solution of 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2,4,5,6-tetrahydropyrrolo[3,4-c]pyrazole-3-carboxylic acid (30 mg, 1 equiv) in DMF (1 mL) was added DIPEA (12.0 mg, 2 equiv) dimethylamine (24.8 mg, 3 equiv) and HATU (53.0 mg, 3 equiv). The mixture was stirred at 25° C. for 1 hr. The reaction mixture was concentrated and purified by prep-HPLC [column: Waters Xbridge 150×25 mm×5 um; mobile phase: [water (ammonia hydroxide v/v)-ACN]; B %: 33%-63%, 8.5 min] to afford the title compound (5.6 mg, 18.5% yield) as a gray solid; ¹H NMR (400 MHZ, DMSO-d₆) δ=9.42 (s, 1H), 7.73 (dd, J=6.0, 8.9 Hz, 1H), 7.35-7.27 (m, 2H), 7.04 (br d, J=2.0 Hz, 1H), 5.39-5.19 (m, 3H), 4.97 (br d, J=1.6 Hz, 2H), 4.28-4.03 (m, 3H), 3.37 (br d, J=2.4 Hz, 6H), 3.12 (br s, 2H), 3.03 (br s, 2H), 2.19-2.04 (m, 4H), 1.94-1.69 (m, 4H), 0.73 (br t, J=7.2 Hz, 3H), LCMS (ESI, M+1): m/z=673.1.

### Example 636

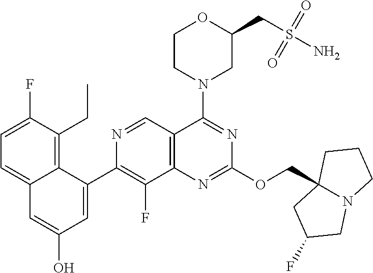 [SMILES: CCc1c(F)ccc2cc(O)cc(-c3ncc4c(N5CCO[C@@H](CS(N)(=O)=O)C5)nc(OC[C@@]56CCCN5C[C@H](F)C6)nc4c3F)c12]

### 1-((R)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) morpholin-2-yl)methanesulfonamide

The title compound was synthesized according to the procedure described for example 541 to produce the desired compound as a yellow solid; ¹H NMR (400 MHZ, DMSO-d₆) δ=9.15 (d, J=2.0 Hz, 1H), 7.77 (dd, J=6.0, 8.8 Hz, 1H), 7.42-7.26 (m, 2H), 7.08-6.85 (m, 3H), 5.42-5.14 (m, 1H), 4.62 (br d, J=13.2 Hz, 1H), 4.40 (br d, J=14.0 Hz, 1H), 4.23-4.12 (m, 2H), 4.11-4.01 (m, 2H), 3.79 (br t, J=11.2 Hz, 1H), 3.61-3.50 (m, 1H), 3.47-3.39 (m, 2H), 3.11-2.98 (m, 3H), 2.82 (br d, J=5.6 Hz, 1H), 2.33 (br s, 2H), 2.18-1.95 (m, 5H), 1.86-1.75 (m, 3H), 0.72 (br t, J=7.2 Hz, 3H), LCMS (ESI, M+1): m/z=673.3.

### Example 637

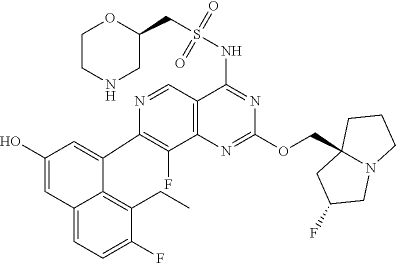 [SMILES: CCc1c(F)ccc2cc(O)cc(-c3ncc4c(NS(=O)(=O)C[C@H]5CNCCO5)nc(OC[C@@]56CCCN5C[C@H](F)C6)nc4c3F)c12]

### N-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-((R)-morpholin-2-yl)methanesulfonamide

The title compound was synthesized according to the procedure described for example 544 to produce the desired compound as a white solid (0.53 formic acid salt); ¹H NMR (400 MHZ, DMSO-d₆) δ=9.02 (s, 1H), 8.15 (s, 1H), 7.75 (dd, J=6.0, 9.2 Hz, 1H), 7.41-7.25 (m, 2H), 6.98 (d, J=2.4 Hz, 1H), 5.45-5.19 (m, 1H), 4.16-4.12 (m, 2H), 3.91 (br dd, J=2.4, 12.4 Hz, 2H), 3.73-3.64 (m, 3H), 3.51 (br s, 2H), 3.33-3.26 (m, 2H), 3.14 (br s, 2H), 3.00-2.79 (m, 4H), 2.69 (s, 1H), 2.25-2.05 (m, 4H), 1.93-1.78 (m, 3H), 0.72 (t, J=7.2 Hz, 3H), LCMS (ESI, M+1): m/z=673.3.

### Example 638

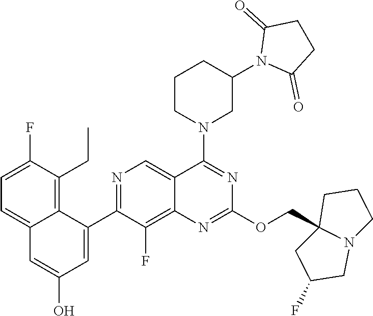 [SMILES: CCc1c(F)ccc2cc(O)cc(-c3ncc4c(N5CCCC(N6C(=O)CCC6=O)C5)nc(OC[C@@]56CCCN5C[C@H](F)C6)nc4c3F)c12]

### 1-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperidin-3-yl)pyrrolidine-2,5-dione

The title compound was synthesized according to the procedure described for example 544, except for the heating of the mixture was conducted at 40° C. for 15 hours, to produce the desired compound as a yellow solid. ¹HNMR (400 MHZ, methanol-d4) δ=9.08 (s, 1H), 7.68 (dd, J=9.2, 5.6 Hz, 1H), 7.30 (d, J=2.4 Hz, 1H), 7.25 (t, J=9.2 Hz, 1H), 7.05 (dd, J=6.0, 2.4 Hz, 1H), 5.23-5.40 (m, 1H), 4.58-4.71 (m, 2H), 4.32-4.48 (m, 2H), 4.20 (dd, J=16.4, 10.4 Hz, 1H), 3.93-4.04 (m, 1H), 3.49-3.62 (m, 1H), 3.12-3.27 (m, 3H), 2.98-3.05 (m, 1H), 2.71 (s, 4H), 2.56-2.66 (m, 1H), 2.42-2.54 (m, 1H), 2.07-2.36 (m, 4H), 1.77-2.02 (m, 6H), 0.75-0.84 (m, 3H); LCMS (ESI, M+1): m/z=675.2.

### Example 639

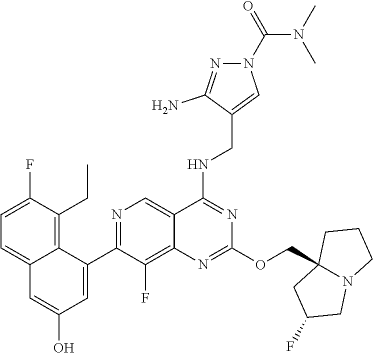 [SMILES: CCc1c(F)ccc2cc(O)cc(-c3ncc4c(NCc5cn(C(=O)N(C)C)nc5N)nc(OC[C@@]56CCCN5C[C@H](F)C6)nc4c3F)c12]

### 3-amino-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide

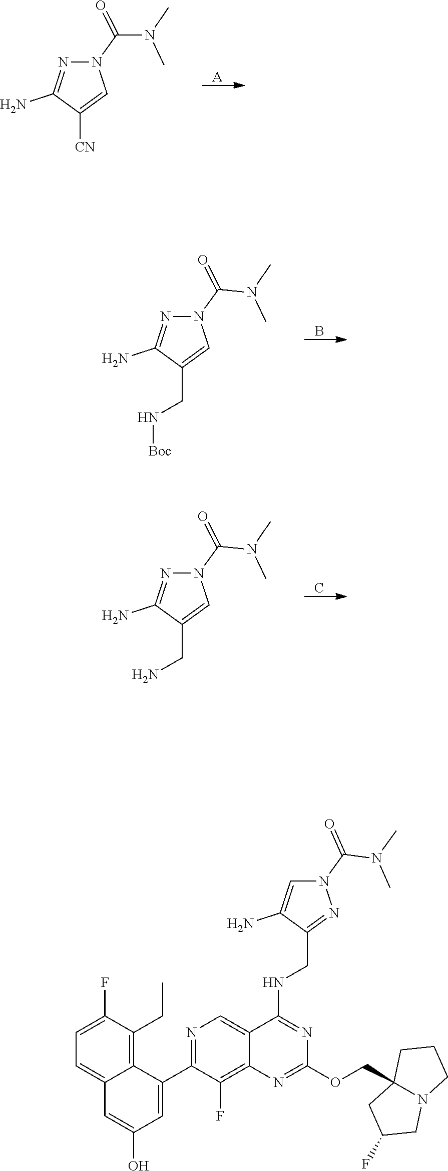

Step A. tert-butyl ((3-amino-1-(dimethylcarbamoyl)-1H-pyrazol-4-yl)methyl)carbamate: To a mixture of Intermediate 15 (500 mg, 1.0 equiv) in MeOH (5.00 mL) was added (Boc)₂O (1.83 g, 3.0 equiv) and Pd/C (150 mg, 10 equiv) under nitrogen atmosphere. The mixture was degassed and purged with hydrogen 3 times. The reaction was stirred under hydrogen (15 psi) at 25° C. for 13 hours. The mixture was filtered and purified by reversed phase flash chromatography [C18, 0.1% formic acid condition] to afford the title compound (460 mg, 58% yield) as a white solid; ¹H NMR (400 MHZ, dimethylsulfoxide-d₆) δ=7.68 (s, 1H), 5.19 (s, 2H), 3.84 (br d, J=6.0 Hz, 2H), 3.08 (s, 6H), 1.38 (s, 9H); LCMS (ESI, M+1): m/z=284.0.

Step B. 3-amino-4-(aminomethyl)-N,N-dimethyl-1H-pyrazole-1-carboxamide: To a mixture of tert-butyl ((3-amino-1-(dimethylcarbamoyl)-1H-pyrazol-4-yl)methyl)carbamate (400 mg, 1.0 equiv) in DCM (4.00 mL) was added TFA (6.44 g, 40 equiv) drop-wise at 0° C. The reaction was stirred at 25° C. for 0.5 hour. The mixture was concentrated to afford the title compound (250 mg, 91% yield) as a yellow oil; ¹H NMR (400 MHZ, dimethylsulfoxide-d₆) δ=8.00 (s, 1H), 3.82 (br d, J=5.2 Hz, 2H), 3.10 (s, 8H).

Step C. 3-amino-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) and 3-amino-4-(aminomethyl)-N,N-dimethyl-1H-pyrazole-1-carboxamide (155 mg, 10 equiv) in DMF (2.0 mL) were added DIPEA (109 mg, 10 equiv) and 4 Å molecular sieves (50.0 mg). The reaction was stirred at 40° C. for 18 hours. The mixture was filtered and purified with prep-HPLC [Phenomenex Luna C18 150×25 mm×10 μm; A: water (FA), B: ACN; B %: 18%-48% over 15 min] to afford the title compound (6.45 mg, 11% yield) as a white solid; ¹H NMR (400 MHZ, dimethylsulfoxide-d₆) δ=9.93 (s, 1H), 9.36-9.24 (m, 2H), 7.93 (s, 1H), 7.76 (dd, J=6.0, 9.2 Hz, 1H), 7.39-7.30 (m, 2H), 6.99 (d, J=2.4 Hz, 1H), 5.50-5.40 (m, 2H), 5.39-5.20 (m, 1H), 4.58-4.41 (m, 2H), 4.25-4.00 (m, 2H), 3.19-2.76 (m, 10H), 2.37-2.30 (m, 1H), 2.22-1.95 (m, 4H), 1.92-1.74 (m, 3H), 0.70 (t, J=7.2 Hz, 3H); ¹⁹F NMR (400 MHZ, dimethylsulfoxide-d₆) δ=−119.672, −139.430, −172.020; LCMS (ESI, M+1): m/z=676.2.

### Example 640

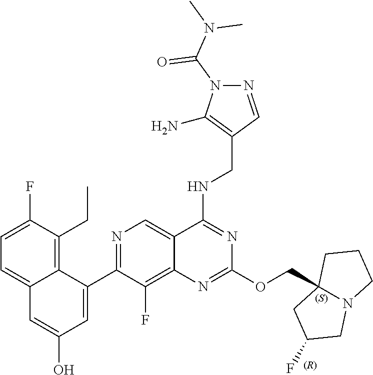

### 5-amino-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide

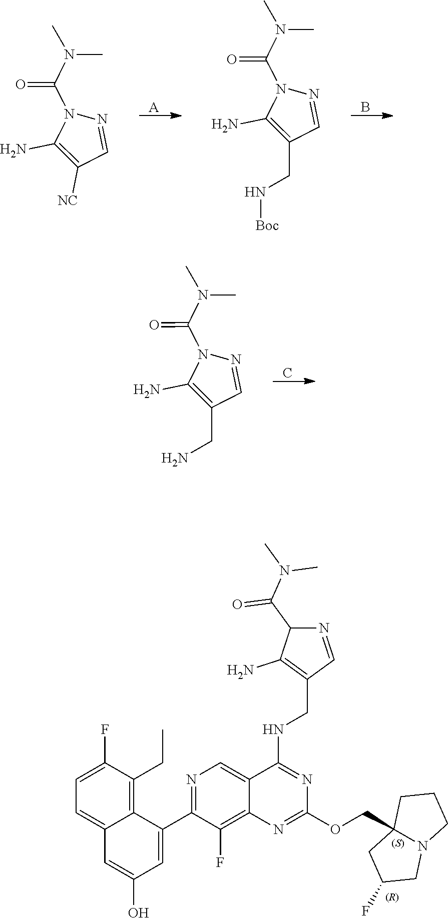

Step A. tert-butyl ((5-amino-1-(dimethylcarbamoyl)-1H-pyrazol-4-yl)methyl)carbamate; To a mixture of 5-amino-4-cyano-N,N-dimethyl-1H-pyrazole-1-carboxamide (500 mg, 1.0 equiv) in MeOH (5.00 mL) was added (Boc)₂O (1.83 g, 3.0 equiv) and Pd/C (150 mg, 10 equiv) under nitrogen atmosphere. The mixture was degassed and purged with hydrogen 3 times. The reaction was stirred under hydrogen (15 psi) at 25° C. for 13 hours. The mixture was filtered and purified by reversed phase flash chromatography [C18, 0.1% formic acid condition] to afford the title compound (460 mg, 58% yield) as a white solid.

Step B. 5-amino-4-(aminomethyl)-N,N-dimethyl-1H-pyrazole-1-carboxamide: To a mixture of tert-butyl ((5-amino-1-(dimethylcarbamoyl)-1H-pyrazol-4-yl)methyl)carbamate (130 mg, 1.0 equiv) in ACN (1.30 mL) was added HCl·dioxane (1.15 mL, 10 equiv) drop-wise at 0° C. The reaction was stirred at 25° C. for 1 hour. The mixture was filtered and concentrated to afford the title compound (90.0 mg, 98% yield) as a yellow oil; ¹H NMR (400 MHZ, dimethylsulfoxide-d₆) δ=7.46 (s, 1H), 3.76 (q, J=5.6 Hz, 2H), 3.04 (br s, 8H).

Step C. 5-amino-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) and 5-amino-4-(aminomethyl)-N,N-dimethyl-1H-pyrazole-1-carboxamide (77.3 mg. 5.0 equiv) in DMF (2.0 mL) were added DIPEA (109 mg, 10 equiv) and 4 Å molecular sieves (50.0 mg). The reaction was stirred at 40° C. for 18 hours. The mixture was filtered and purified with prep-HPLC [Phenomenex Luna C18 150×25 mm×10 μm; A: water (FA), B: ACN; B %: 15%-45% over 10 min] to afford the title compound (8.83 mg, 15% yield) as a white solid (0.77 formic acid salt); ¹H NMR (400 MHz, dimethylsulfoxide-d₆) δ=9.34 (br d, J=3.2 Hz, 1H), 9.29 (s, 1H), 7.76 (dd, J=6.0, 8.8 Hz, 1H), 7.41 (s, 1H), 7.40-7.27 (m, 2H), 6.99 (d, J=2.0 Hz, 1H), 6.08 (s, 2H), 5.40-5.16 (m, 1H), 4.44 (br d, J=5.2 Hz, 2H), 4.25-4.01 (m, 2H), 3.18-2.98 (m, 10H), 2.89-2.79 (m, 1H), 2.36-2.29 (m, 1H), 2.12-1.99 (m, 3H), 1.88-1.75 (m, 3H), 0.70 (br t, J=7.2 Hz, 3H). LCMS (ESI, M+1): m/z=676.3

### Example 641

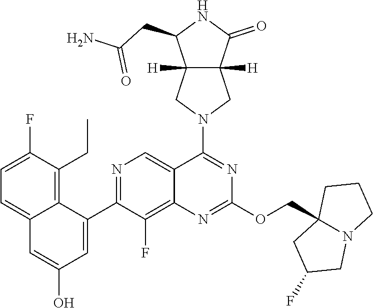 [SMILES: CCc1c(F)ccc2cc(O)cc(-c3ncc4c(N5C[C@@H]6[C@@H](CC(N)=O)NC(=O)[C@@H]6C5)nc(OC[C@@]56CCCN5C[C@H](F)C6)nc4c3F)c12]

### 2-((1R,3aS,6aR)-5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxooctahydropyrrolo[3,4-c]pyrrol-1-yl)acetamide

The title compound was synthesized using Intermediate 13C according to the procedure described for example 566, except for heating of the mixture was conducted at 60° C., to produce the desired compound as a white solid (0.1 formic acid salt); ¹H NMR (400 MHZ, METHANOL-d₄) δ=9.30 (d, J=5.2 Hz, 1H), 8.53 (s, 1H), 7.68 (dd, J=5.6, 9.2 Hz, 1H), 7.32-7.22 (m, 2H), 7.05 (dd, J=2.8, 5.2 Hz, 1H), 5.46-5.27 (m, 1H), 4.60-4.50 (m, 1H), 4.48-4.27 (m, 4H), 4.02-3.95 (m, 2H), 3.46-3.36 (m, 2H), 3.18-3.07 (m, 2H), 2.66 (s, 2H), 2.60 (d, J=7.0 Hz, 2H), 2.53-2.52 (m, 1H), 2.54-2.34 (m, 1H), 2.33-2.26 (m, 1H), 2.22-2.13 (m, 2H), 2.09-2.03 (m, 2H), 1.99-1.91 (m, 1H), 0.79 (q, J=7.2 Hz, 3H); LCMS (ESI, M+1): m/z=676.5.

### Example 642

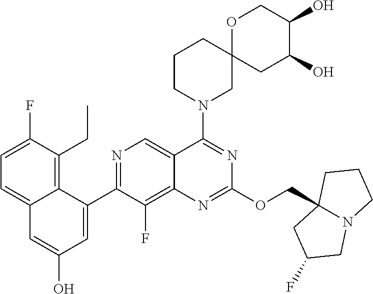 [SMILES: CCc1c(F)ccc2cc(O)cc(-c3ncc4c(N5CCCC6(C[C@H](O)[C@H](O)CO6)C5)nc(OC[C@@]56CCCN5C[C@H](F)C6)nc4c3F)c12]

### (3R,4S)-8-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-1-oxa-8-azaspiro[5.5]undecane-3,4-diol

The title compound was synthesized according to the procedure described for example 538 to produce the desired compound as a yellow solid; ¹H NMR (400 MHZ, methanol-d₄) δ=9.23 (d, J=5.2 Hz, 1H), 7.78-7.65 (m, 1H), 7.34 (s, 1H), 7.28 (t, J=9.2 Hz, 1H), 7.14-7.03 (m, 1H), 5.72-5.41 (m, 1H), 4.77-4.51 (m, 5H), 4.01-3.38 (m, 11H), 2.61-2.29 (m, 6H), 2.27-2.06 (m, 4H), 1.89-1.70 (m, 4H), 0.82 (br t, J=6.8 Hz, 3H); LCMS (ESI, M+1): m/z=680.3.

### Example 643

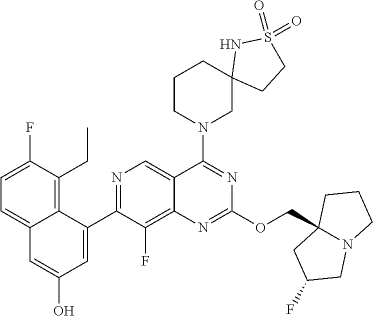 [SMILES: CCc1c(F)ccc2cc(O)cc(-c3ncc4c(N5CCCC6(CCS(=O)(=O)N6)C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide

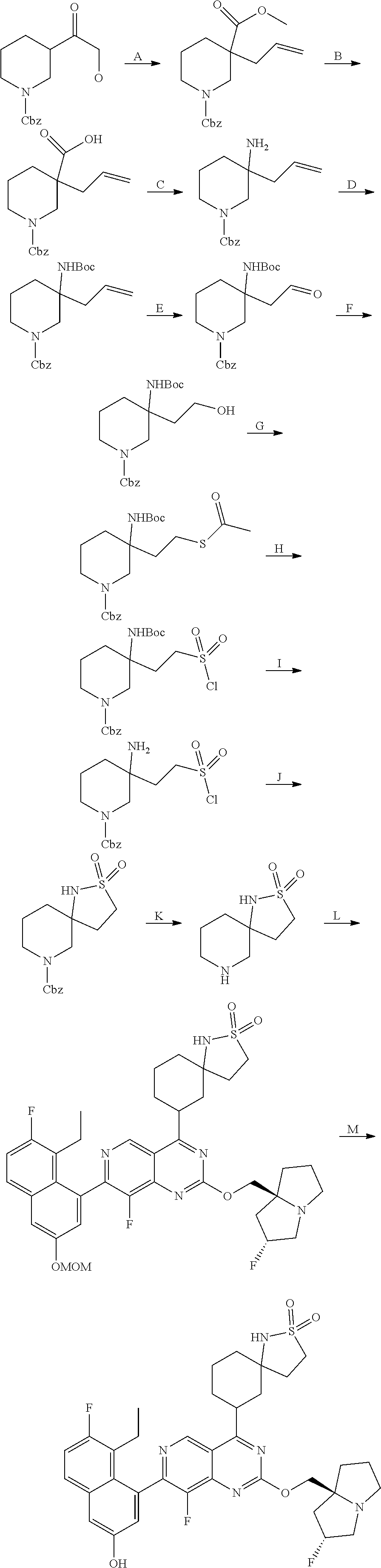

Step A. 1-benzyl 3-methyl 3-allylpiperidine-1,3-dicarboxylate: A solution of 1-benzyl 3-methyl piperidine-1,3-dicarboxylate (50.0 g, 1.0 equiv) in THF (500 mL) was cooled to −78° C. under argon atmosphere. Following that LiHMDS (1 M, 234 mL, 1.3 equiv) was added over a period of 30 minutes. The reaction was stirred at −78° C. for 1 hour. To the reaction mixture was added 3-bromoprop-1-ene (26.2 g, 1.2 equiv) at −78° C. The reaction was stirred at 20° C. for 5 hours. The reaction was quenched by saturated aqueous NH₄Cl (200 mL) and water (100 mL). The mixture was extracted with ethyl acetate (3×200 mL) and washed with 10% aqueous citric acid (2×200 mL). The combined organic layers were washed with brine (200 mL) and dried over anhydrous sodium sulfate. The residue was concentrated to afford the title compound (55.0 g, 96% yield) as a brown oil; ¹H NMR (400 MHZ, CHLOROFORM-d) δ=7.47-7.28 (m, 5H), 5.81-5.54 (m, 1H), 5.19-5.00 (m, 4H), 4.00 (br d, J=13.2 Hz, 1H), 3.70-3.53 (m, 4H), 3.21 (br d, J=13.4 Hz, 2H), 2.40-2.27 (m, 1H), 2.27-2.16 (m, 1H), 2.05 (s, 1H), 1.60 (br d, J=4.0 Hz, 3H)

Step B. 3-allyl-1-((benzyloxy)carbonyl)piperidine-3-carboxylic acid: A solution of 1-benzyl 3-methyl 3-allylpiperidine-1,3-dicarboxylate (55.0 g, 1.0 equiv) and LiOH·H₂O (29.1 g, 4.0 equiv) in MeOH (300 mL) and H₂O (100 mL) was stirred at 60° C. for 12 hours under N₂ atmosphere. pH of the mixture was adjusted to 2 by adding 2M HCl and extracted with ethyl acetate (500 mL). The organic layer was concentrated to afford the title compound (49.0 g, 93% yield) as a yellow oil; ¹H NMR (400 MHZ, CHLOROFORM-d) δ=7.43-7.27 (m, 5H), 5.87-5.51 (m, 1H), 5.28-4.98 (m, 5H), 3.68-3.52 (m, 1H), 3.37-3.06 (m, 3H), 2.46-2.13 (m, 4H), 2.08-1.98 (m, 2H), 1.68-1.61 (m, 2H), 1.60-1.48 (m, 2H)

Step C. benzyl 3-allyl-3-aminopiperidine-1-carboxylate: To a solution of 3-allyl-1-((benzyloxy)carbonyl)piperidine-3-carboxylic acid (49.0 g, 1.0 equiv), DPPA (48.9 g, 1.1 equiv) and TEA (32.7 g, 2.0 equiv) in toluene (500 mL) was stirred at 80° C. for 4 hours. To the mixture was added 4M HCl·dioxane to adjust pH to 2. The mixture was diluted with water (150 mL) and the layers were separated. To the aqueous layer was added solid NaHCO₃ to adjust pH to 9 and the resulting was extracted with ethyl acetate (2×30 mL). The combined organic layer was washed with brine (40 mL) and dried over sodium sulfate. The residue was concentrated to afford the title compound (43.0 g, 97% yield) as a yellow oil; LCMS (ESI, M−99): m/z=275.2.

Step D. benzyl 3-allyl-3-((tert-butoxycarbonyl)amino)piperidine-1-carboxylate: A solution of benzyl 3-allyl-3-aminopiperidine-1-carboxylate (42.0 g, 1.0 equiv) and (Boc)₂O (334 g, 10 equiv) was stirred at 60° C. for 4 hours under N₂ atmosphere. The mixture was concentrated and purified by column chromatography [SiO₂, Petroleum ether/Ethyl acetate 12:1] to afford the title compound (36.0 g, 62% yield) as a white solid; LCMS (ESI, M−99): m/z=319.1.

Step E. benzyl 3-((tert-butoxycarbonyl)amino)-3-(2-oxoethyl)piperidine-1-carboxylate: To a solution of benzyl 3-allyl-3-((tert-butoxycarbonyl)amino)piperidine-1-carboxylate (4.00 g, 1.0 equiv) in THF (40 mL) and H₂O (40 mL) was added NaIO₄ (4.57 g, 2.0 equiv) and K₂OsO₄×2H₂O (197 mg, 0.05 equiv) portionwise at 0° C. The mixture was stirred at 20° C. for 2 hours. The mixture was quenched by adding saturated Na₂S₂O₃ aqueous solution (2×50 mL). The mixture was extracted with ethyl acetate (3×50 mL). The organic layer was dried over Na₂SO₄ and concentrated under vacuum. The residue was purified by column chromatography [SiO₂, Petroleum ether/Ethyl acetate 10:1 to 2:1] to afford the title compound (1.70 g, 41% yield) as a white solid; ¹H NMR (400 MHZ, CHLOROFORM-d) δ=9.79 (br s, 1H), 7.40-7.35 (m, 4H), 7.34-7.30 (m, 1H), 5.29-5.04 (m, 2H), 4.16-3.85 (m, 2H), 3.20-2.71 (m, 4H), 1.78-1.47 (m, 4H), 1.41 (s, 9H); LCMS (ESI, M−99): m/z=277.2.

Step F. benzyl 3-((tert-butoxycarbonyl)amino)-3-(2-hydroxyethyl)piperidine-1-carboxylate: To a solution of benzyl 3-((tert-butoxycarbonyl)amino)-3-(2-oxoethyl)piperidine-1-carboxylate (5.60 g, 1.0 equiv) in EtOH (30 mL) and THF (30 mL) was added NaBH₄ (1.18 g, 2.1 equiv) portionwise at 0° C. The mixture was stirred at 0° C. for 1 hour. The mixture was quenched by adding sat. aq. NH₄Cl (30 mL) slowly and extracted with ethyl acetated (100 mL). The organic layer was concentrated and purified by column chromatography [SiO₂, Petroleum ether/Ethyl acetate 10:1 to 1:1] to afford the title compound (5.30 g, 93% yield) as a white solid; ¹H NMR (400 MHz, CHLOROFORM-d) δ=7.41-7.35 (m, 4H), 7.35-7.29 (m, 1H), 5.28-5.05 (m, 2H), 4.98-4.65 (m, 1H), 4.11-3.96 (m, 1H), 3.90-3.63 (m, 3H), 3.14-2.94 (m, 2H), 2.65-2.33 (m, 1H), 1.74-1.45 (m, 4H), 1.42 (s, 9H); LCMS (ESI, M−99): m/z=279.1.

Step G. benzyl 3-(2-(acetylthio)ethyl)-3-((tert-butoxycarbonyl)amino)piperidine-1-carboxylate: To a solution of PPh₃ (3.05 g. 2.0 equiv) in THF (40 mL) was added DIAD (2.35 g, 2.0 equiv) at 0° C. The mixture was stirred at 0° C. for 0.5 hour. To the mixture was added a solution of benzyl 3-((tert-butoxycarbonyl)amino)-3-(2-hydroxyethyl)piperidine-1-carboxylate (2.20 g, 1.0 equiv) and ethanethioic S-acid (885 mg, 2.0 equiv) in THF (20 mL) slowly at 0° C. The mixture was stirred at 0° C. for 1 hour and then at 15° C. for 1 hour. The mixture was diluted with water (100 mL) and extracted with ethyl acetate (200 mL). The organic layer was concentrated and purified by column chromatography [SiO₂, Petroleum ether/Ethyl acetate 20:1 to 7:1] to afford the title compound (5.00 g, crude) as a white solid; LCMS (ESI, M−99): m/z=337.1.

Step H. benzyl 3-((tert-butoxycarbonyl)amino)-3-(2-(chlorosulfonyl)ethyl)piperidine-1-carboxylate: To a solution of benzyl 3-(2-(acetylthio)ethyl)-3-((tert-butoxycarbonyl)amino)piperidine-1-carboxylate (1.00 g, 1.0 equiv) in AcOH (6 mL) and H₂O (0.6 mL) was added NCS (918 mg, 3.0 equiv). The mixture was stirred at 15° C. for 0.5 hour. The mixture was diluted with water (50 mL) and extracted with ethyl acetate (50 mL). The organic phase was dried over Na₂SO₄ and concentrated to afford the title compound (1.5 g, crude) as a yellow oil.

Step I. benzyl 3-amino-3-(2-(chlorosulfonyl)ethyl)piperidine-1-carboxylate: To a solution of benzyl 3-((tert-butoxycarbonyl)amino)-3-(2-(chlorosulfonyl)ethyl)piperidine-1-carboxylate (1.50 g, 1.0 equiv) in DCM (2 mL) was added TFA (7.70 g, 21 equiv) at 0° C. The mixture was stirred at 0° C. for 15 minutes. The mixture was concentrated to afford the title compound (1.2 g, crude) as a yellow oil.

Step J. benzyl 2-thia-1,7-diazaspiro[4.5]decane-7-carboxylate 2,2-dioxide: To a solution of benzyl 3-amino-3-(2-(chlorosulfonyl)ethyl)piperidine-1-carboxylate (1.2 g, crude) in THF (12 mL) was added K₂CO₃ (2.00 g, 14.5 mmol). The mixture was stirred at 15° C. for 2 hours. The mixture was added water (10 mL) and extracted with ethyl acetate (20 mL). The organic layer was dried over Na₂SO₄ and concentrated to give a residue. The residue was purified by reversed phase flash chromatography [C18, 0.1% formic acid condition] and column chromatography [SiO₂, Petroleum ether/Ethyl acetate 5:1 to 1:2] to afford the title compound (100 mg, 26% yield over four steps) as a yellow oil; ¹H NMR (400 MHZ, CHLOROFORM-d) δ=7.42-7.29 (m, 5H), 5.23-5.06 (m, 2H), 4.55-4.24 (m, 1H), 3.72-3.29 (m, 4H), 3.18 (br s, 2H), 2.37-2.09 (m, 2H), 1.89 (br s, 1H), 1.78-1.63 (m, 3H); LCMS (ESI, M+1): m/z=325.2.

Step K. 2-thia-1,7-diazaspiro[4.5]decane 2,2-dioxide: To a suspension of Pd/C (30.0 mg, 10% purity) in MeOH (2 mL) was added benzyl 2-thia-1,7-diazaspiro[4.5]decane-7-carboxylate 2,2-dioxide (100 mg, 1.0 equiv) under N₂. The suspension was degassed under vacuum and purged with H₂ several times. The mixture was stirred under H₂ (15 psi) at 15° C. for 3 hours. The mixture was filtered and concentrated to afford the title compound (70 mg, crude) as a yellow gum.

Step L. 7-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide: To a mixture of 7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (130 mg, 1.0 equiv), 2-thia-1,7-diazaspiro[4.5]decane 2,2-dioxide (57.9 mg, 1.5 equiv) and 4 Å molecular sieves (20.0 mg) in DMF (0.6 mL) was added DIPEA (79.2 mg, 3.0 equiv). The mixture was stirred at 40° C. for 10 hours. The mixture was filtered and purified by reversed phase flash chromatography [C18, 0.1% formic acid condition] to afford the title compound (110 mg, 74% yield) as a white solid; LCMS (ESI, M+1): m/z=727.3.

Step M. 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide: To a solution of 7-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,7-diazaspiro[4.5]decane 2,2-dioxide (100 mg, 1.0 equiv) in ACN (1 mL) was added HCl·dioxane (4 M, 2 mL, 58 equiv) at 0° C. The mixture was stirred at 0° C. for 15 minutes. The mixture was concentrated and diluted with sat. NaHCO₃ to adjust the pH to 9. The mixture was extracted with ethyl acetate (2×20 mL). The organic layer was concentrated and purified by prep-HPLC [column: Waters Xbridge 150×25 mm×5 μm; A: water (NH₄HCO₃), B: ACN, B %: 36%-66% over 9 min] to afford the title compound (30.7 mg, 32% yield) as a white solid; ¹H NMR (400 MHZ, DMSO-d₆) δ=9.92 (br s, 1H), 9.11 (d, J=3.6 Hz, 1H), 7.76 (ddd, J=3.2, 6.0, 9.2 Hz, 1H), 7.41-7.36 (m, 1H), 7.35-7.29 (m, 2H), 7.03-6.98 (m, 1H), 5.42-5.15 (m, 1H), 4.27-3.97 (m, 4H), 3.89-3.44 (m, 2H), 3.31-3.05 (m, 5H), 3.04-2.99 (m, 1H), 2.88-2.76 (m, 1H), 2.41-2.27 (m, 1H), 2.25 (m, 3H), 2.07 (m, 4H), 1.89-1.70 (m, 5H), 0.72 (t, J=7.2 Hz, 3H); LCMS (ESI, M+1): m/z=683.3.

### Example 644

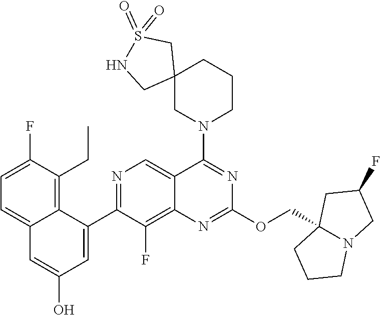 [SMILES: CCc1c(F)ccc2cc(O)cc(-c3ncc4c(N5CCCC6(CNS(=O)(=O)C6)C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-3,7-diazaspiro[4.5]decane 2,2-dioxide

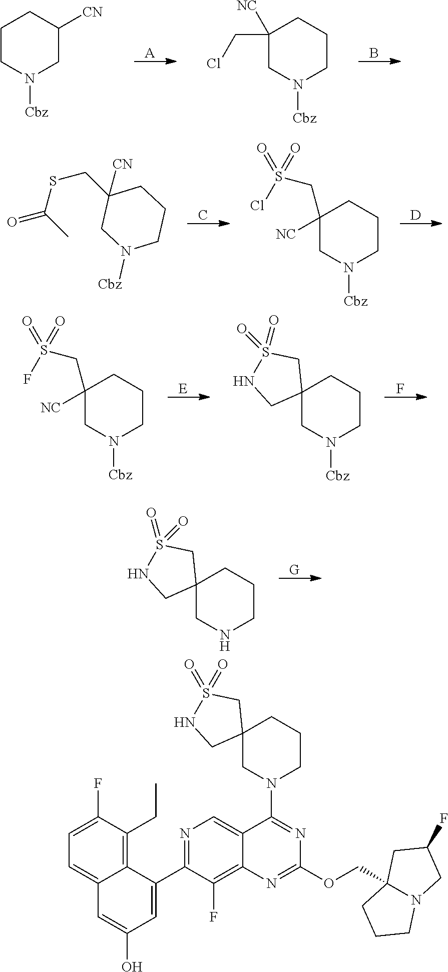

Step A. benzyl 3-(chloromethyl)-3-cyanopiperidine-1-carboxylate: To a solution of benzyl 3-cyanopiperidine-1-carboxylate (5.00 g, 1.0 equiv) in tetrahydrofuran (250 mL) was added dropwise LiHMDS (22.5 mL, 1.0 M, 1.1 equiv) at −78° C. and the resulting was stirred for 1 hour. Then chloro(iodo) methane (4.69 g, 1.3 equiv) in tetrahydrofuran (30 mL) was added dropwise at −78° C. and the mixture was stirred at −78° C. for 1 hour. The reaction mixture was allowed to warm up to 20° C. and stirred for 12 hours. The reaction mixture was quenched with aq. ammonium chloride (6 mL) at 0° C. and diluted with water (200 mL). The mixture was extracted with ethyl acetate (2×200 mL). The combined organic layers were dried over anhydrous sodium sulfate, concentrated and purified with column chromatography [SiO₂, petroleum ether/ethyl acetate 50:1 to 10:1] to afford the title compound (5.00 g, 76% yield) as a colorless oil. LCMS (ESI, M+1): m/z=293.2.

Step B. benzyl 3-((acetylthio)methyl)-3-cyanopiperidine-1-carboxylate: To a solution of acetylsulfanylpotassium (2.73 g, 1.4 equiv) in DMF (90 mL) was added benzyl 3-(chloromethyl)-3-cyanopiperidine-1-carboxylate (5.00 g, 1.0 equiv). The mixture was stirred at 85° C. for 12 hours. The reaction mixture was quenched with water (200 mL) at 0° C., and then diluted with water (200 mL) and extracted with ethyl acetate (2×200 mL). The combined organic layer was dried over anhydrous Na₂SO₄, concentrated and purified by column chromatography [SiO₂, petroleum ether/ethyl acetate 10:1 to 0:1] to afford the title compound (5.20 g, 89% yield) as a red oil. LCMS (ESI, M+1): m/z=333.1.

Step C. benzyl 3-((chlorosulfonyl)methyl)-3-cyanopiperidine-1-carboxylate: To a solution of NCS (8.03 g, 4.0 equiv) in HCl (7.52 mL, 2.0 M, 1.0 equiv) was added dropwise benzyl 3-(acetylsulfanylmethyl)-3-cyano-piperidine-1-carboxylate (5.00 g, 1.0 equiv) in MeCN (15 mL) at 10° C. The mixture was stirred at 25° C. for 0.5 hour. The residue was diluted with water (10 mL) and extracted with ethyl acetate (3×20 mL). The combined organic layers were dried over anhydrous sodium sulfate and concentrated to afford the title compound (6.20 g, 95% yield) as white solid. LCMS (ESI, M+1): m/z=356.0.

Step D. benzyl 3-cyano-3-((fluorosulfonyl)methyl)piperidine-1-carboxylate: To a solution of benzyl 3-((chlorosulfonyl)methyl)-3-cyanopiperidine-1-carboxylate (6.20 g, 1.0 equiv) in MeCN (10 mL) were added KF (1.67 g, 2.0 equiv) and 18-CROWN-6 (1.14 g, 0.3 equiv). The mixture was stirred at 25° C. for 14 hours. The residue was diluted with water (10 mL) and extracted with ethyl acetate (3×10 mL). The combined organic layers were dried over anhydrous sodium sulfate, concentrated and purified with column chromatography [SiO₂, petroleum ether/ethyl acetate 1:0 to 2:1] to afford the title compound (4.10 g, 84% yield) as a white solid. ¹H NMR (400 MHz, methanol-d₄) δ=7.27-7.44 (m, 6H), 5.14 (br d, J=5.6 Hz, 3H), 4.84 (s, 3H), 4.18-4.33 (m, 1H), 3.64-3.78 (m, 1H), 3.37-3.58 (m, 1H), 2.76-3.05 (m, 2H), 2.45-2.64 (m, 1H), 2.14-2.37 (m, 1H), 2.10-2.13 (m, 1H), 1.75-1.93 (m, 2H).

Step E. benzyl 2-thia-3,7-diazaspiro[4.5]decane-7-carboxylate 2,2-dioxide: A mixture of benzyl 3-cyano-3-((fluorosulfonyl)methyl)piperidine-1-carboxylate (3.60 g, 1.0 equiv) and NiCl₂·6H₂O (2.77 g, 1.1 equiv) in MeOH (5 mL) was cooled to −20° C. Then NaBH₄ (1.56 g, 3.9 equiv) was added portionwise at −20° C. The reaction mixture was warmed up to 25° C. and stirred for 14 hours. The reaction mixture was quenched with anhydrous ammonium chloride (10 mL). The residue was diluted with water (100 mL) and extracted with ethyl acetate (2×50.0 mL). The combined organic layers were dried over anhydrous sodium sulfate, concentrated and purified by prep-HPLC [neutral condition, column: Waters X bridge 150×25 mm×5 μm; mobile phase: water(NH₄HCO₃)-ACN]; B %: 25%-55%, 10 minutes] to afford the title compound (140 mg, 3.8% yield) as a white solid. LCMS (ESI, M+1): m/z=325.2.

Step F. 2-thia-3,7-diazaspiro[4.5]decane 2,2-dioxide: To a solution of benzyl 2-thia-3,7-diazaspiro[4.5]decane-7-carboxylate 2,2-dioxide (140 mg, 1.0 equiv) in MeOH (2 mL) was added Pd/C (30.0 mg, 0.6 equiv.). The reaction mixture was degassed and purged with H₂ for three times. The mixture was stirred under H₂ (15 Psi) at 20° C. for 2 hours. The reaction mixture was filtered, concentrated and purified by prep-HPLC [neutral condition, column: Waters X bridge 150×25 mm×5 μm; mobile phase: water (NH₄HCO₃)-ACN]; B %: 1%-10%, 10 minutes] to afford the title compound (33.0 mg, 40% yield) as a white solid. LCMS (ESI, M): m/z=190.7.

Step G. 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-3,7-diazaspiro[4.5]decane 2,2-dioxide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (25.0 mg, 1.0 equiv) and 2-thia-3,7-diazaspiro[4.5]decane 2,2-dioxide (24.1 mg, 3.0 equiv) in DMF (1 mL) was added 4 Å molecular sieves (20.0 mg) and DIPEA (54.5 mg, 10 equiv). The mixture was stirred at 40° C. for 24 hours. The mixture was filtered and purified with prep-HPLC [neutral condition, column: Waters X bridge 150×25 mm×5 μm; mobile phase: water (NH₄HCO₃)-ACN]; B %: 28%-58%, 10 minutes] to afford the title compound (5.13 mg, 17.3% yield) as a yellow solid. ¹H NMR (400 MHZ, methanol-d₄) δ=9.09 (s, 1H), 7.67 (dd, J=8.8, 6.0 Hz, 1H), 7.30 (d, J=2.0 Hz, 1H), 7.25 (t, J=9.2 Hz, 1H), 6.99-7.09 (m, 1H), 5.21-5.41 (m, 1H), 4.50-4.69 (m, 1H), 4.39 (dd, J=10.8, 5.6 Hz, 1H), 4.18-4.35 (m, 2H), 3.86-4.02 (m, 1H), 3.67-3.86 (m, 1H), 3.33-3.41 (m, 1H), 3.13-3.28 (m, 3H), 2.96-3.12 (m, 3H), 2.32-2.55 (m, 2H), 2.11-2.25 (m, 3H), 1.94-2.04 (m, 4H), 1.75-1.93 (m, 4H), 0.80 (q, J=7.2 Hz, 3H); LCMS (ESI, M+1): m/z=683.5.

### Example 645

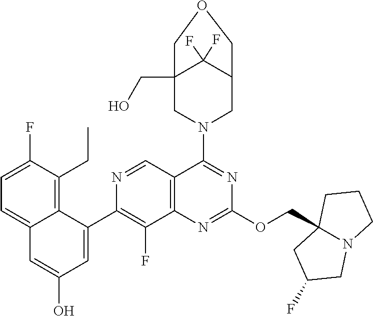 [SMILES: CCc1c(F)ccc2cc(O)cc(-c3ncc4c(N5CC6COCC(CO)(C5)C6(F)F)nc(OC[C@@]56CCCN5C[C@H](F)C6)nc4c3F)c12]

### 4-(4-(9,9-difluoro-1-(hydroxymethyl)-3-oxa-7-azabicyclo[3.3.1]nonan-7-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 544 to produce the desired compound as a yellow solid. ¹H NMR (400 MHZ, METHANOL-d4) δ=9.21 (s, 1H), 7.69 (dd, J=6.0, 9.2 Hz, 1H), 7.32 (d, J=2.4 Hz, 1H), 7.26 (t, J=9.6 Hz, 1H), 7.05 (br d, J=1.6 Hz, 1H), 5.42-5.27 (m, 1H), 4.75 (br d, J=2.4 Hz, 2H), 4.48-4.40 (m, 3H), 4.23 (br dd, J=3.2, 11.6 Hz, 1H), 4.10-4.02 (m, 2H), 3.67 (td, J=4.2, 13.2 Hz, 1H), 3.45-3.33 (m, 3H), 3.29-3.21 (m, 3H), 3.08 (dt, J=6.4, 9.6 Hz, 1H), 2.45-2.32 (m, 2H), 2.31-2.26 (m, 1H), 2.25-2.08 (m, 4H), 2.03-1.88 (m, 2H), 0.76 (t, J=7.2 Hz, 3H); LCMS (ESI, M+1): m/z=686.2.

### Example 646

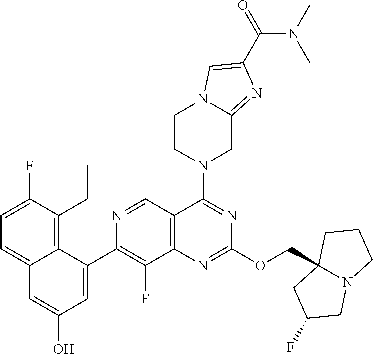 [SMILES: CCc1c(F)ccc2cc(O)cc(-c3ncc4c(N5CCn6cc(C(=O)N(C)C)nc6C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydroimidazo[1,2-a]pyrazine-2-carboxamide

The title compound was synthesized according to the procedure described for example 544 to produce the desired compound as a white solid (0.90 formic acid salt); ¹H NMR (400 MHZ, METHANOL-d4) δ=9.21 (s, 1H), 8.55 (s, 1H), 7.68 (dd, J=6.0, 9.2 Hz, 1H), 7.61 (s, 1H), 7.31 (d, J=2.4 Hz, 1H), 7.25 (t, J=9.2 Hz, 1H), 7.06 (d, J=2.4 Hz, 1H), 5.39-5.23 (m, 3H), 4.55-4.47 (m, 2H), 4.45 (br s, 2H), 4.40-4.31 (m, 2H), 3.41-3.35 (m, 3H), 3.12-3.02 (m, 4H), 2.52-2.43 (m, 1H), 2.35-2.09 (m, 5H), 2.07-1.85 (m, 4H), 0.80 (t, J=7.4 Hz, 3H); LCMS (ESI, M+1): m/z=687.3

### Example 647

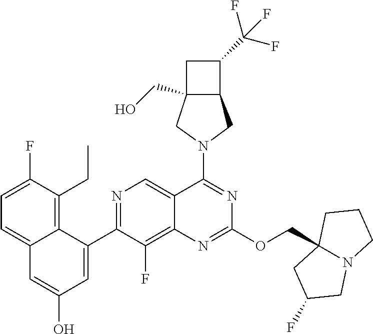 [SMILES: CCc1c(F)ccc2cc(O)cc(-c3ncc4c(N5C[C@@H]6[C@@H](C(F)(F)F)C[C@]6(CO)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-((1R,5R,6S)-1-(hydroxymethyl)-6-(trifluoromethyl)-3-azabicyclo[3.2.0]heptan-3-yl)pyrido[4,3-d]pyrimidin-7-yl)naphthalen-2-ol

The title compound was synthesized according to the procedure described for example 550 except for 4 Å molecular sieves (10.0 mg) were added to the mixture, to produce the desired compound as a yellow solid (0.31 formic acid salt); ¹H NMR (400 MHZ, methanol-d4) δ=9.31 (s, 1H), 8.50 (s, 1H), 7.68 (dd, J=9.2, 6.0 Hz, 1H), 7.31 (d, J=2.4 Hz, 1H), 7.25 (t, J=9.6 Hz, 1H), 7.05 (d, J=2.0 Hz, 1H), 5.31-5.51 (m, 1H), 4.38-4.56 (m, 4H), 4.04-4.25 (m, 2H), 3.69 (s, 2H), 3.40-3.64 (m, 3H), 3.09-3.23 (m, 2H), 2.99 (dt, J=16.8, 8.4 Hz, 1H), 2.09-2.54 (m, 9H), 1.94-2.06 (m, 1H), 0.80 ppm (q, J=6.0 Hz, 3H); LCMS (ESI, M+1): m/z=688.4.

### Example 648

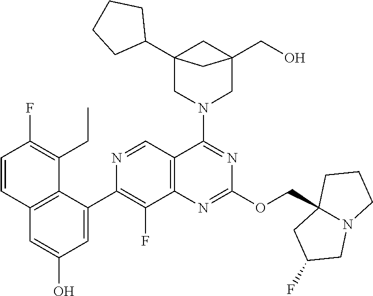 [SMILES: CCc1c(F)ccc2cc(O)cc(-c3ncc4c(N5CC6(CO)CC(C7CCCC7)(C5)C6)nc(OC[C@@]56CCCN5C[C@H](F)C6)nc4c3F)c12]

### 4-(4-(1-cyclopentyl-5-(hydroxymethyl)-3-azabicyclo[3.1.1]heptan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol

The title compound was synthesized according to the procedure described for example 544 to produce the desired compound as a white solid; ¹H NMR (400 MHZ, DMSO-d6) δ=9.46 (s, 1H), 7.73 (dd, J=6.0, 8.9 Hz, 1H), 7.40-7.22 (m, 2H), 7.01 (d, J=2.4 Hz, 1H), 5.37-5.17 (m, 1H), 4.23-4.05 (m, 4H), 3.43 (s, 3H), 3.14-2.95 (m, 4H), 2.82 (br d, J=6.0 Hz, 1H), 2.21-2.10 (m, 2H), 2.08-1.99 (m, 2H), 1.95-1.74 (m, 6H), 1.68 (br s, 4H), 1.60-1.49 (m, 4H), 1.41-1.26 (m, 4H), 0.73 (br t, J=7.2 Hz, 3H), LCMS (ESI, M+1): m/z=688.5.

### Example 649

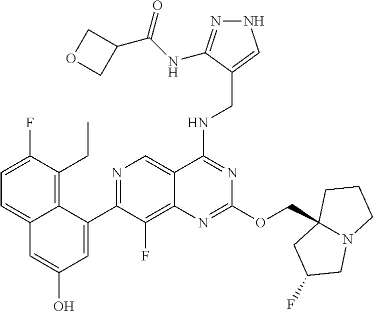 [SMILES: CCc1c(F)ccc2cc(O)cc(-c3ncc4c(NCc5c[nH]nc5NC(=O)C5COC5)nc(OC[C@@]56CCCN5C[C@H](F)C6)nc4c3F)c12]

### N-(4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1H-pyrazol-3-yl) oxetane-3-carboxamide

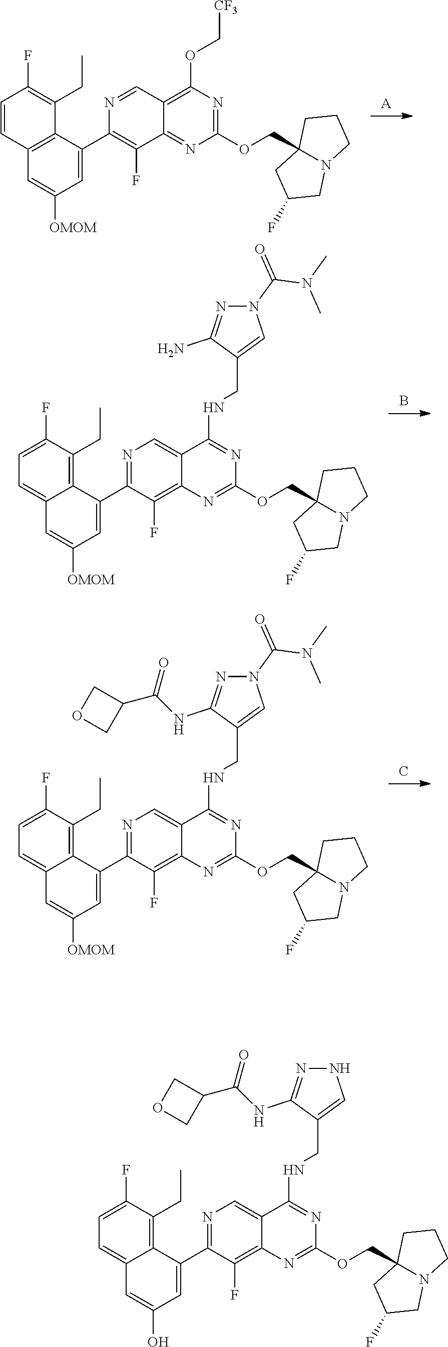

Step A. 3-amino-4-(((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide: To a mixture of 7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (730 mg, 1.0 equiv) and K₃PO₄ (730 mg, 3 equiv) in ACN (3.5 mL) and DMF (3.5 mL) was added 3-amino-4-(aminomethyl)-N,N-dimethyl-1H-pyrazole-1-carboxamide (420 mg, 2.0 equiv) and 4 Å molecular sieves (100 mg). The reaction was stirred at 40° C. for 12 hrs. The mixture was filtered, diluted with water (10 mL) and extracted with ethyl acetate (3×15 mL). The combined organic layers were dried over anhydrous sodium sulfate, concentrated, and purified by reversed phase flash chromatography [C18, 0.1% formic acid condition] to afford the title compound (640 mg, 74% yield) as a yellow oil; ¹H NMR (400 MHZ, dimethylsulfoxide-d₆) δ=9.32-9.29 (m, 2H), 7.94-7.86 (m, 2H), 7.68-7.65 (m, 1H), 7.48-7.38 (m, 1H), 7.19 (d, J=2.0 Hz, 1H), 5.47-5.30 (m, 4H), 5.24-5.18 (m, 1H), 4.57-4.43 (m, 2H), 4.21-4.14 (m, 1H), 4.13-4.06 (m, 1H), 3.47-3.38 (m, 3H), 3.14-3.06 (m, 7H), 3.04-3.00 (m, 1H), 2.87-2.79 (m, 1H), 2.43-2.30 (m, 1H), 2.09 (br s, 2H), 2.08-2.03 (m, 1H), 2.03-1.99 (m, 1H), 1.88-1.73 (m, 3H), 0.77 (br s, 3H); LCMS (ESI, M+1): m/z=720.3.

Step B. 4-(((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-3-(oxetane-3-carboxamido)-1H-pyrazole-1-carboxamide: To a mixture of oxetane-3-carboxylic acid (125 mg, 2.0 equiv) in DMF (6 mL) were added HOBt (124 mg, 1.5 equiv) and EDCI (176 mg, 1.5 equiv). After stirring at 20° C. for 2 hours, 3-amino-4-(((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide (440 mg, 1.0 equiv) was added. The reaction was stirred at 20° C. for 16 hours. The mixture was diluted with water (10 ml) and extracted with EtOAc (3×20 mL). The combined organic layers were dried over anhydrous sodium sulfate, concentrated, and purified by reversed phase flash chromatography [C18, 0.1% formic acid condition] to afford the title compound (102 mg, 20% yield) as a yellow oil; LCMS (ESI, M+1): m/z=804.3.

Step C N-(4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1H-pyrazol-3-yl) oxetane-3-carboxamide: To a mixture of 4-(((7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-3-(oxetane-3-carboxamido)-1H-pyrazole-1-carboxamide (92 mg, 1.0 equiv) in DCM (0.2 mL) was added TFA (196 mg, 15 equiv). The reaction was stirred at 20° C. for 0.5 hour. The pH of the mixture was adjusted to 6 with sodium bicarbonate, filtered, and purified with prep-HPLC [Waters xbridge C18 150×25 mm×5 μm; A: water (NH₄HCO₃), B: ACN, B %: 27%-57% over 8 min] and lyophilized to afford the title compound (1.71 mg, 2.0% yield) as an off-white solid (0.67 formic acid salt); SFC: >99% ee, Chiralpak IC-3 50×4.6 mm I.D., 3 μm, Isocratic elution: 50% MeOH (0.05% DEA) in CO₂, 3 mL/min; 220 nm, t_{R}: 0.662 min; ¹H NMR (400 MHZ, methanol-d₄) δ=9.11 (s, 1H), 8.55 (s, 1H), 7.71-7.63 (m, 1H), 7.50 (s, 1H), 7.30 (d, J=2.8 Hz, 1H), 7.24 (t, J=9.2 Hz, 1H), 7.03 (d, J=2.8 Hz, 1H), 5.43-5.37 (m, 0.5H), 5.23 (br d, J=4.8 Hz, 0.5H), 4.67-4.54 (m, 3H), 4.39-4.25 (m, 3H), 3.97-3.82 (m, 2H), 3.28-3.12 (m, 4H), 3.11-2.99 (m, 2H), 2.50-2.37 (m, 2H), 2.10-1.94 (m, 5H), 0.95-0.84 (m, 3H); ¹⁹F NMR (400 MHZ, methanol-d₄) δ=−76.948, −121.228, −138.977, −173.771; LCMS (ESI, M+1): m/z=689.1.

### Example 650

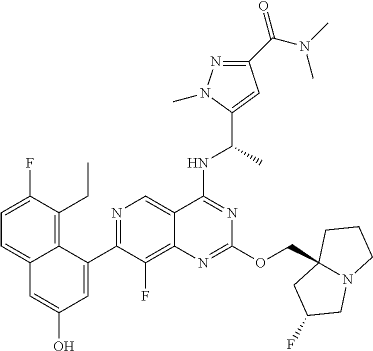 [SMILES: CCc1c(F)ccc2cc(O)cc(-c3ncc4c(N[C@@H](C)c5cc(C(=O)N(C)C)nn5C)nc(OC[C@@]56CCCN5C[C@H](F)C6)nc4c3F)c12]

### 5-((S)-1-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)ethyl)-N,N,1-trimethyl-1H-pyrazole-3-carboxamide

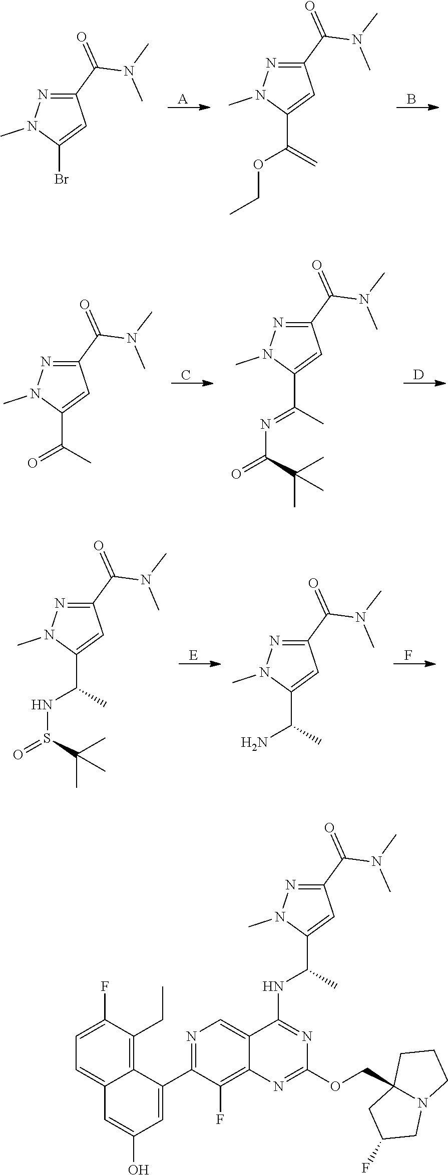

Step A. 5-(1-ethoxyvinyl)-N,N,1-trimethyl-1H-pyrazole-3-carboxamide: To a mixture of 5-bromo-N,N,1-trimethyl-1H-pyrazole-3-carboxamide (0.3 g, 1.0 equiv) and tributyl(1-ethoxyvinyl) stannane (700 mg, 1.5 equiv) in toluene (5.0 mL) was added Pd(PPh₃)₂Cl₂ (90.7 mg, 0.1 equiv) in one portion under N₂. The mixture was degassed and stirred at 100° C. for 12 hours. The reaction mixture was diluted by water (10 mL) extracted with ethyl acetate (3×10 mL), dried over anhydrous sodium sulfate, filtered and concentrated to afford the title compound (288 mg, 99% yield) as a black oil; LCMS (ESI, M+1): m/z=224.0.

Step B. 5-acetyl-N,N,1-trimethyl-1H-pyrazole-3-carboxamide: To a mixture of 5-(1-ethoxyvinyl)-N,N,1-trimethyl-1H-pyrazole-3-carboxamide (280 mg, 1.0 equiv) in THF (9.4 mL) was added HCl (4 N, 6.0 equiv) at 25° C. under nitrogen. The mixture was degassed and purged with nitrogen 3 times. The reaction was stirred at 25° C. for 2 hours. The reaction mixture was diluted with water (20 mL) and extracted with ethyl acetate (3×30 mL). The combined organic layers were dried over anhydrous sodium sulfate, filtered, concentrated and purified by reversed phase flash chromatography [water (0.1% formic acid)/acetonitrile)] to afford the title compound (60 mg, 24% yield) as a white solid; LCMS (ESI, M+1): m/z=196.1.

Step C. (R,E)-5-(1-((tert-butylsulfinyl)imino)ethyl)-N,N,1-trimethyl-1H-pyrazole-3-carboxamide: To a mixture of 5-acetyl-N,N,1-trimethyl-1H-pyrazole-3-carboxamide (60.0 mg, 1.0 equiv) and (R)-2-methylpropane-2-sulfinamide (44.7 mg, 1.2 equiv) in THF (10 mL) was added tetraisopropoxytitanium (262 mg, 3.0 equiv) at 25° C. under nitrogen. The mixture was degassed and purged with nitrogen 3 times. The reaction was heated to 80° C. and stirred for 12 hours. The reaction mixture was diluted with water (10 mL) and extracted with ethyl acetate (3×5 mL). The combined organic layers were dried over anhydrous sodium sulfate, filtered and concentrated to afford the title compound (90 mg, 98% yield) as a white solid; LCMS (ESI, M+1): m/z=299.1.

Step D. 5-((S)-1-((R)-1,1-dimethylethylsulfinamido)ethyl)-N,N,1-trimethyl-1H-pyrazole-3-carboxamide: To a mixture of (R,E)-5-(1-((tert-butylsulfinyl)imino)ethyl)-N,N,1-trimethyl-1H-pyrazole-3-carboxamide (330 mg, 1.0 equiv) in THF (3 mL) was added NaBH₄ (83.7 mg, 2.0 equiv) portionwise at −15° C. under N₂. The reaction was degassed and stirred at 0° C. for 2 hours. The reaction mixture was acidified with HCl (2 N) to pH=5 and extracted with ethyl acetate (3×10 mL). The combined organic layers were dried over anhydrous sodium sulfate, filtered and concentrated and purified by reversed phase flash chromatography [water (0.1% formic acid)/acetonitrile)] followed by SFC [DAICEL Chiralpak IC 250 mm×30 mm, 10 μm; A: (0.1% NH₃·H₂O MeOH); B %: 30%-30% over 8.1; 113 min] to afford the title compound (60 mg, 18% yield) as a yellow oil; LCMS (ESI, M+1): m/z=300.9.

Step E. (S)-5-(1-aminoethyl)-N,N,1-trimethyl-1H-pyrazole-3-carboxamide: To a mixture of 5-((S)-1-((R)-1,1-dimethylethylsulfinamido)ethyl)-N,N,1-trimethyl-1H-pyrazole-3-carboxamide (50 mg, 1.0 equiv) in ACN (0.5 mL) was added HCl·dioxane (4 M, 0.5 mL, 12 equiv) at 0° C. under N₂. The reaction was degassed and stirred at 20° C. for 1 hour. The reaction mixture was concentrated to afford the title compound (50 mg, 98% yield) as a white solid; LCMS (ESI, M+1): m/z=197.0.

Step F. 5-((S)-1-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)ethyl)-N,N,1-trimethyl-1H-pyrazole-3-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50 mg, 1.0 equiv) and(S)-5-(1-aminoethyl)-N,N,1-trimethyl-1H-pyrazole-3-carboxamide (30 mg, 1.16 equiv) in DMF (0.01 mL) was added DIPEA (218 mg, 1.69 mmol, 20 equiv) under nitrogen. The reaction was degassed and purged with nitrogen 3 times. The reaction was stirred at 40° C. for 24 hours. The mixture was purified by prep-HPLC [Phenomenex luna C18 150×25 mm×10 μm; A: water (FA), B: ACN, B %: 18%-48% over 10 min] and lyophilized to afford the title compound (7.73 mg, 12% yield) as a yellow solid (0.96 formic acid salt); ¹H NMR (400 MHZ, dimethylsulfoxide-d₆) δ=9.40 (d, J=4.0 Hz, 1H), 9.30-9.18 (m, 1H), 7.82-7.70 (m, 1H), 7.34 (s, 1H), 7.32 (d, J=2.0 Hz, 1H), 6.98 (d, J=2.0 Hz, 1H), 6.65 (d, J=13.2 Hz, 1H), 5.71 (br t, J=7.2 Hz, 1H), 5.42-5.16 (m, 1H), 4.17-4.04 (m, 2H), 4.00-3.90 (m, 3H), 3.30 (br s, 3H), 3.08 (br s, 2H), 2.99 (br s, 1H), 2.95 (br s, 3H), 2.86-2.79 (m, 1H), 2.38-2.31 (m, 1H), 2.16-1.96 (m, 4H), 1.87-1.72 (m, 3H), 1.66 (br t, J=6.4 Hz, 3H), 0.75-0.67 (m, 3H); ¹⁹F NMR (400 MHZ, dimethylsulfoxide-d₆) δ=−119.694, −139.400, −171.990; LCMS (ESI, M+1): m/z=689.3.

### Example 651

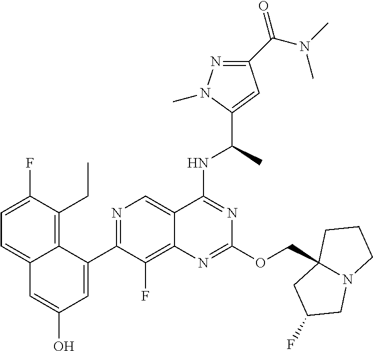 [SMILES: CCc1c(F)ccc2cc(O)cc(-c3ncc4c(N[C@H](C)c5cc(C(=O)N(C)C)nn5C)nc(OC[C@@]56CCCN5C[C@H](F)C6)nc4c3F)c12]

### 5-((R)-1-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)ethyl)-N,N,1-trimethyl-1H-pyrazole-3-carboxamide

The title compound was synthesized according to the 6-step procedure described for example 650 except for(S)-2-methylpropane-2-sulfinamide was used in step C instead of (R)-2-methylpropane-2-sulfinamide to produce the desired compound as a white solid (0.37 formic acid salt); ¹H NMR (400 MHz, dimethylsulfoxide-d₆) δ=10.0-9.85 (m, 1H), 9.39 (d, J=4.0 Hz, 1H), 9.25 (br t, J=8.4 Hz, 1H), 7.80-7.71 (m, 1H), 7.39-7.34 (m, 1H), 7.32 (d, J=2.0 Hz, 1H), 6.98 (d, J=2.4 Hz, 1H), 6.65 (d, J=13.2 Hz, 1H), 5.77-5.65 (m, 1H), 5.39-5.17 (m, 1H), 4.20-4.11 (m, 1H), 4.10-4.00 (m, 1H), 3.94 (d, J=15.6 Hz, 3H), 3.28 (br s, 3H), 3.12-3.01 (m, 3H), 2.99-2.92 (m, 3H), 2.86-2.79 (m, 1H), 2.17-1.96 (m, 5H), 1.88-1.75 (m, 3H), 1.66 (t, J=6.4 Hz, 3H), 0.76-0.64 (m, 3H); ¹⁹F NMR (400 MHZ, dimethylsulfoxide-d₆) δ=−119.667, −139.414, −171.986; LCMS (ESI, M+1): m/z=689.4.

### Example 652

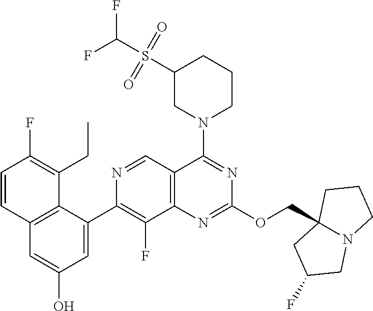 [SMILES: CCc1c(F)ccc2cc(O)cc(-c3ncc4c(N5CCCC(S(=O)(=O)C(F)F)C5)nc(OC[C@@]56CCCN5C[C@H](F)C6)nc4c3F)c12]

### 4-(4-(3-((difluoromethyl) sulfonyl) piperidin-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

The title compound was synthesized according to the procedure described for example 550 except for heating of the mixture was conducted at 80° C. for 5 hours, to produce the desired compound as a white solid; ¹H NMR (400 MHZ, METHANOL-d₄) δ=0.79 (q, J=7.2 Hz, 3H) 1.87 (br s, 2H) 1.96-2.04 (m, 2H) 2.07-2.30 (m, 6H) 2.34-2.50 (m, 3H) 2.96-3.05 (m, 1H) 3.18-3.27 (m, 4H) 3.71-3.88 (m, 1H) 3.90-4.01 (m, 1H) 4.30-4.37 (m, 2H) 4.97 (br d, J=14.0 Hz, 1H) 5.31 (br d, J=53.2 Hz, 1H) 6.86 (td, J=52.0, 7.6 Hz, 1H) 7.06 (dd, J=11.2, 2.4 Hz, 1H) 7.21-7.28 (m, 1H) 7.30 (d, J=2.4 Hz, 1H) 7.68 (dd, J=8.8, 6.0 Hz, 1H) 9.10 (d, J=3.2 Hz, 1H); LCMS (ESI, M+1): m/z=692.3.

### Example 653

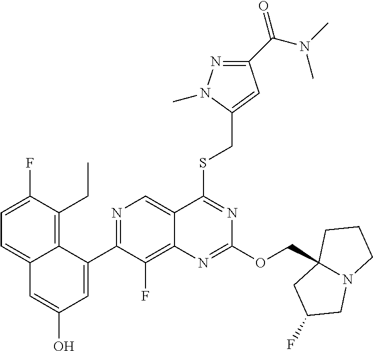 [SMILES: CCc1c(F)ccc2cc(O)cc(-c3ncc4c(SCc5cc(C(=O)N(C)C)nn5C)nc(OC[C@@]56CCCN5C[C@H](F)C6)nc4c3F)c12]

### 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)thio)methyl)-N,N,1-trimethyl-1H-pyrazole-3-carboxamide

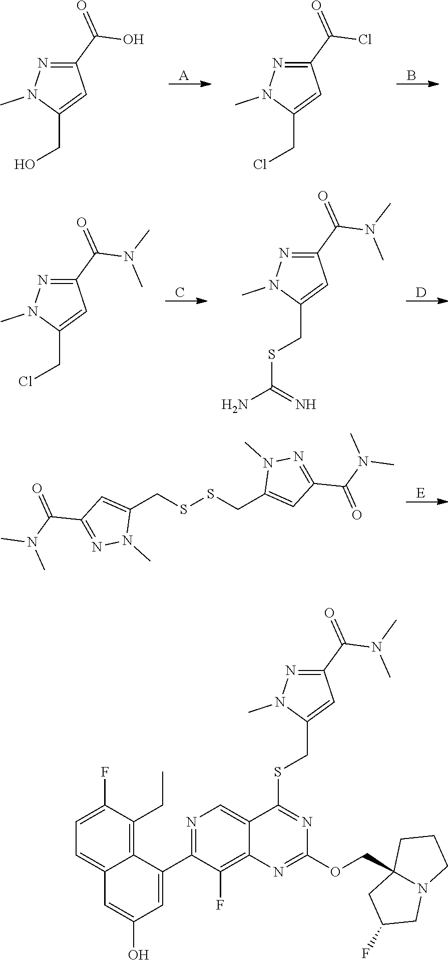

Step A. 5-(chloromethyl)-1-methyl-1H-pyrazole-3-carbonyl chloride: A solution of 5-(hydroxymethyl)-1-methyl-1H-pyrazole-3-carboxylic acid (480 mg, 1.0 equiv) in SOCl₂ (7.87 g, 4.80 mL, 5.0 equiv) was stirred at 80° C. for 1 hour. The mixture was concentrated to afford the title compound (500 mg, crude) as a yellow oil.

Step B. 5-(chloromethyl)-N,N,1-trimethyl-1H-pyrazole-3-carboxamide: To a solution of 5-(chloromethyl)-1-methyl-1H-pyrazole-3-carbonyl chloride (500 mg, 1.0 equiv) in DCM (4 mL) was added N-methylmethanamine (2 M in THF, 7.77 mL, 6.0 equiv) at 0° C. The reaction was stirred at 0° C. for 0.5 hour. The mixture was concentrated and purified by silica gel chromatography (petroleum ether/ethyl acetate 50/1 to 1/1) to afford the title compound (350 mg, 66% yield) as a white solid; LCMS (ESI, M+1): m/z=201.9.

Step C. (3-(dimethylcarbamoyl)-1-methyl-1H-pyrazol-5-yl)methyl carbamimidothioate: To a solution of 5-(chloromethyl)-N,N,1-trimethyl-pyrazole-3-carboxamide (100 mg, 1.0 equiv) in DMSO (1 mL) was added thiourea (113 mg, 3.0 equiv). The reaction was stirred at 25° C. for 6 hours and then concentrated to afford the title compound (100 mg, crude) as a yellow solid.

Step D. 5,5′-(disulfanediylbis(methylene)bis(N,N, 1-trimethyl-1H-pyrazole-3-carboxamide): A solution of (3-(dimethylcarbamoyl)-1-methyl-1H-pyrazol-5-yl)methyl carbamimidothioate (250 mg, 1.0 equiv), K₂CO₃ (430 mg, 3.0 equiv) in DMSO (1 mL) was stirred at 25° C. for 2 hours. The reaction mixture was filtered and concentrated. The residue was purified by reversed phase flash chromatography [water (0.1% FA)/acetonitrile] to afford the title compound (55 mg, 12% yield) as white solid; LCMS (ESI, M+1): m/z=397.3.

Step E. 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)thio)methyl)-N,N, 1-trimethyl-1H-pyrazole-3-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (40.0 mg, 1.0 equiv) and 5,5′-(disulfanediylbis(methylene))bis(N,N, 1-trimethyl-1H-pyrazole-3-carboxamide) (53.5 mg, 2.0 equiv) in DMF (0.1 mL) were added tributylphosphine (27.3 mg, 2.0 equiv), 4 Å molecular sieves (5 mg, 1.0 equiv) and DIPEA (175 mg, 20.0 equiv). The reaction was stirred at 40° C. for 16 hours. The reaction mixture was concentrated and purified by prep-HPLC [Welch Xtimate C18 150×25 mm×5 μm; A: water (NH₃·H₂O), B: ACN, B %: 38%-68% over 8 min] to afford the title compound (7.89 mg, 16% yield) as a yellow solid; SFC: Chiralpak AS-3 50×4.6 mm I.D., 3 μm, Gradient elution: MeOH (0.05% DEA) in CO₂ from 5% to 40%, 3 mL/min, 220 nm, t_{R}: 1.808 min, 2.018 min; ¹H NMR (400 MHZ, dimethylsulfoxide-d₆+deuterium oxide-d₂) δ=9.23 (s, 1H), 7.76 (dd, J=6.0, 9.2 Hz, 1H), 7.40-7.32 (m, 2H), 7.03 (d, J=2.4 Hz, 1H), 6.65 (s, 1H), 5.40-5.20 (m, 1H), 4.78 (s, 2H), 4.27-4.17 (m, 2H), 4.00 (s, 3H), 3.23 (s, 2H), 3.08 (br d, J=6.8 Hz, 2H), 3.01 (s, 1H), 2.93 (s, 3H), 2.86-2.79 (m, 1H), 2.57-2.55 (m, 1H), 2.3-2.24 (m, 1H), 2.22-2.12 (m, 1H), 2.11-1.96 (m, 3H), 1.89-1.70 (m, 3H), 0.67 (t, J=7.6 Hz, 3H); ¹⁹F NMR (400 MHZ, dimethylsulfoxide-d₆+deuterium oxide-d₂) δ=−119.262, −138.040, −171.834; LCMS (ESI, M+1): m/z=692.2.

### Example 654

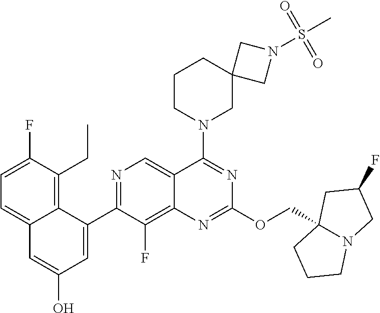 [SMILES: CCc1c(F)ccc2cc(O)cc(-c3ncc4c(N5CCCC6(C5)CN(S(C)(=O)=O)C6)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(2-(methylsulfonyl)-2,6-diazaspiro[3.5]nonan-6-yl)pyrido[4,3-d]pyrimidin-7-yl)naphthalen-2-ol

The title compound was synthesized according to the procedure described for example 538 to produce the desired compound as a yellow solid. ¹H NMR (400 MHZ, METHANOL-d₄) δ=9.06 (s, 1H), 7.65 (dd, J=5.9, 9.2 Hz, 1H), 7.27 (d, J=2.4 Hz, 1H), 7.22 (t, J=9.2 Hz, 1H), 7.05 (d, J=2.4 Hz, 1H), 5.39-5.22 (m, 1H), 4.36-4.19 (m, 4H), 4.12-3.96 (m, 2H), 3.82 (dd, J=7.0, 13.6 Hz, 2H), 3.71 (dd, J=2.1, 8.0 Hz, 2H), 3.28-3.11 (m, 3H), 2.97 (s, 3H), 2.54-2.42 (m, 1H), 2.40-2.27 (m, 1H), 2.27-2.22 (m, 1H), 2.22-2.11 (m, 3H), 2.04-1.92 (m, 5H), 1.85-1.77 (m, 2H), 0.80 (dt, J=1.7, 7.2 Hz, 3H). LCMS (ESI, M+1): 697.3.

### Example 655

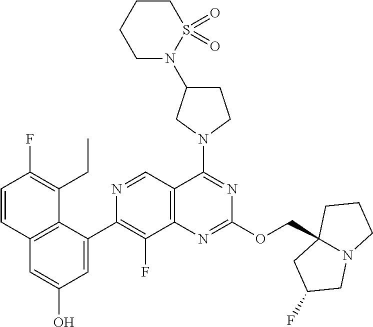 [SMILES: CCc1c(F)ccc2cc(O)cc(-c3ncc4c(N5CCC(N6CCCCS6(=O)=O)C5)nc(OC[C@@]56CCCN5C[C@H](F)C6)nc4c3F)c12]

### 2-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)pyrrolidin-3-yl)-1,2-thiazinane 1,1-dioxide

The title compound was synthesized according to the procedure described for example 538 to produce the desired compound as a white solid. ¹H NMR (400 MHZ, METHANOL-d₄) δ=9.26 (d, J=7.2 Hz, 1H), 8.53 (s, 1H), 7.68 (dd, J=6.0, 9.2 Hz, 1H), 7.30 (d, J=2.4 Hz, 1H), 7.25 (t, J=9.2 Hz, 1H), 7.05 (t, J=2.4 Hz, 1H), 5.51-5.21 (m, 1H), 4.65 (br d, J=6.8 Hz, 1H), 4.44-4.38 (m, 1H), 4.36-4.31 (m, 1H), 4.28-3.90 (m, 4H), 3.48-3.34 (m, 4H), 3.17-3.07 (m, 3H), 2.48-1.89 (m, 13H), 1.74 (br d, J=3.6 Hz, 2H), 0.78 (td, J=7.2, 10.8 Hz, 3H). LCMS (ESI, M+1): 697.3.

### Example 656

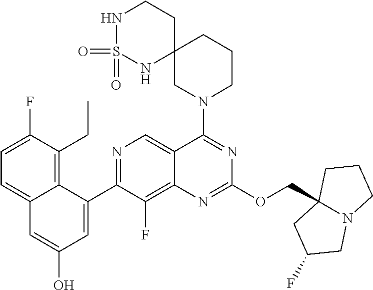 [SMILES: CCc1c(F)ccc2cc(O)cc(-c3ncc4c(N5CCCC6(CCNS(=O)(=O)N6)C5)nc(OC[C@@]56CCCN5C[C@H](F)C6)nc4c3F)c12]

### 8-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,8-triazaspiro[5.5]undecane 2,2-dioxide

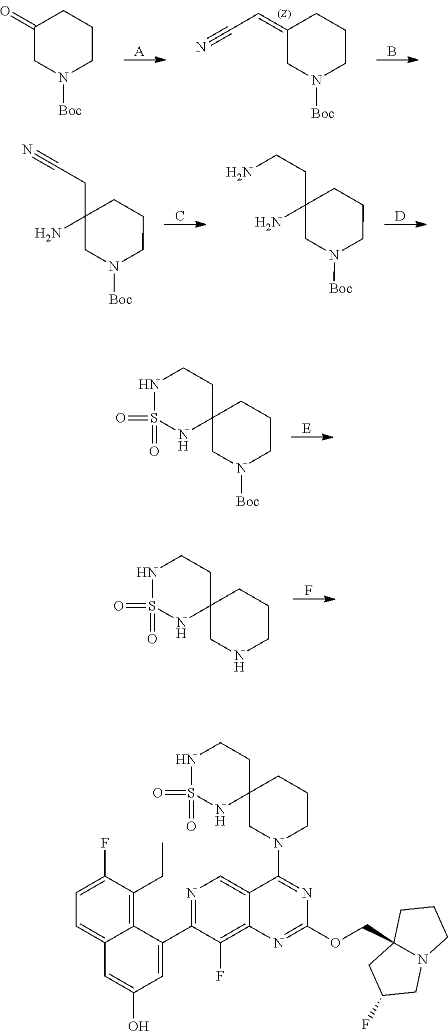

Step A. (Z)-tert-butyl 3-(cyanomethylene)piperidine-1-carboxylate: NaH (2.41 g, 60% purity, 1.2 equiv) was added portionwise to 2-diethoxyphosphorylacetonitrile (10.7 g, 1.2 equiv) in anhydrous THF (200 mL) under hydrogen at 25° C. and stirred for 30 minutes. Following that was added tert-butyl 3-oxopiperidine-1-carboxylate (10 g, 1.0 equiv). The reaction was stirred at 25° C. for 12 hours. The mixture was quenched with water (200 mL) and concentrated to remove THE, extracted with EtOAc (2×300 mL). The combined organic layers were washed with brine, dried over anhydrous sodium sulfate, concentrated, and purified by silica gel chromatography (petroleum ether/ethyl acetate 30:1 to 1:1) to afford the title compound (10 g, 81% yield) as a colorless oil; 1H NMR (400 MHZ, chloroform-d) δ=5.28-5.13 (m, 1H), 4.28-4.22 (m, 1H), 4.02-3.95 (m, 1H), 3.55-3.45 (m, 2H), 2.67-2.58 (m, 1H), 2.46-2.36 (m, 1H), 1.82-1.68 (m, 2H), 1.53-1.41 (m, 9H).

Step B. tert-butyl 3-amino-3-(cyanomethyl)piperidine-1-carboxylate: To a mixture of tert-butyl (3Z)-3-(cyanomethylene)piperidine-1-carboxylate (3 g, 1.0 equiv) in MeOH (9 mL) was added NH₃·MeOH (36 mL, 20% purity). The reaction was heated to 100° C. with stirring for 16 hours in a sealed tube. The mixture was concentrated and purified by Al₂O₃ chromatography (petroleum ether/ethyl acetate 10:1 to 0:1) to afford the title compound (1.08 g, 30% yield) as a yellow oil; 1H NMR (400 MHZ, chloroform-d) δ=3.65-3.03 (m, 4H), 2.64-2.32 (m, 2H), 1.86-1.67 (m, 2H), 1.66-1.58 (m, 2H), 1.48 (s, 9H); LCMS (ESI, M−55): m/z=184.1.

Step C. tert-butyl 3-amino-3-(2-aminoethyl)piperidine-1-carboxylate: To a mixture of tert-butyl 3-amino-3-(cyanomethyl)piperidine-1-carboxylate (1.25 g, 1.0 equiv) in methanol (20 mL) was added Raney-Ni (1.34 g, 3.0 equiv) and NH₃·MeOH (5 mL, 20% purity) under nitrogen. The reaction was degassed under vacuum and purged with hydrogen several times. The reaction was stirred under hydrogen (15 psi) at 20° C. for 16 hours. The mixture was filtered and concentrated to afford the title compound (1.4 g, 99% yield) as a light blue oil; ¹H NMR (400 MHz, dimethylsulfoxide-d₆) δ=3.75-3.19 (m, 4H), 3.14-2.72 (m, 4H), 2.52 (br s, 2H), 1.64-1.11 (m, 15H); LCMS (ESI, M+1): m/z=244.1.

Step D. tert-butyl 2-thia-1,3,8-triazaspiro[5.5]undecane-8-carboxylate 2,2-dioxide: To a mixture of tert-butyl 3-amino-3-(2-aminoethyl)piperidine-1-carboxylate (1.49 g, 1.0 equiv) in pyridine (15 mL) was added sulfuric diamide (706 mg, 1.2 equiv) at 25° C. The reaction was stirred at 125° C. for 12 hours. The mixture was concentrated and purified by prep-TLC (dichloromethane/methanol 10:1) to afford the title compound (443 mg, 23% yield) as a yellow oil; ¹H NMR (400 MHZ, dimethylsulfoxide-d₆) δ=6.54-6.42 (m, 1H), 6.42-6.30 (m, 1H), 4.26-4.16 (m, 1H), 3.78-3.60 (m, 1H), 3.45-3.34 (m, 1H), 3.30-3.21 (m, 1H), 2.93-2.69 (m, 2H), 1.75-1.66 (m, 1H), 1.60-1.42 (m, 4H), 1.42-1.35 (m, 9H), 1.33-1.21 (m, 1H); LCMS (ESI, M−55): m/z=250.2.

Step E. 2-thia-1,3,8-triazaspiro[5.5]undecane 2,2-dioxide; To a mixture of tert-butyl 2-thia-1,3,8-triazaspiro[5.5]undecane-8-carboxylate 2,2-dioxide (200 mg, 1.0 equiv) in ACN (1.23 mL) was added dropwise HCl·dioxane (4 M, 2.46 mL, 15 equiv) at 0° C. The reaction was stirred at 25° C. for 1 hour. The mixture was concentrated to afford the title compound (150 mg, HCl) as a yellow solid; LCMS (ESI, M+1): m/z=206.2.

Step F. 8-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,8-triazaspiro[5.5]undecane 2,2-dioxide: The title compound was synthesized according to the procedure described for example 541 to produce the desired compound as an off-white solid; SFC: Chiralpak IC-3 50×4.6 mm I.D., 3 μm; Gradient elution: 40% MeOH+ACN (0.05% DEA) in CO₂, 3 mL/min, 220 nm, t_{R}: 1.207 mim, 1.996 min; ¹H NMR (400 MHZ, dimethylsulfoxide-d₆) δ=9.96 (s, 1H), 9.14-9.05 (m, 1H), 7.81-7.68 (m, 1H), 7.40-7.25 (m, 2H), 7.04-6.95 (m, 1H), 6.73-6.60 (m, 1H), 6.46-6.35 (m, 1H), 5.37-5.18 (m, 1H), 4.56-3.98 (m, 4H), 3.98-3.66 (m, 2H), 3.36 (br s, 1H), 3.12-2.97 (m, 4H), 2.91-2.51 (m, 1H), 2.39-2.31 (m, 1H), 2.22-1.90 (m, 6H), 1.88-1.70 (m, 5H), 1.65-1.54 (m, 1H), 1.48-1.32 (m, 1H), 0.82-0.65 (m, 3H); ¹⁹F NMR (400 MHZ, dimethylsulfoxide-d₆) δ=−119.619, −139.505, −172.020; LCMS (ESI, M+1): m/z=698.0.

### Example 657

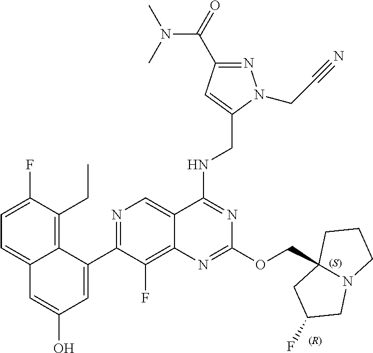

### 1-(cyanomethyl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide

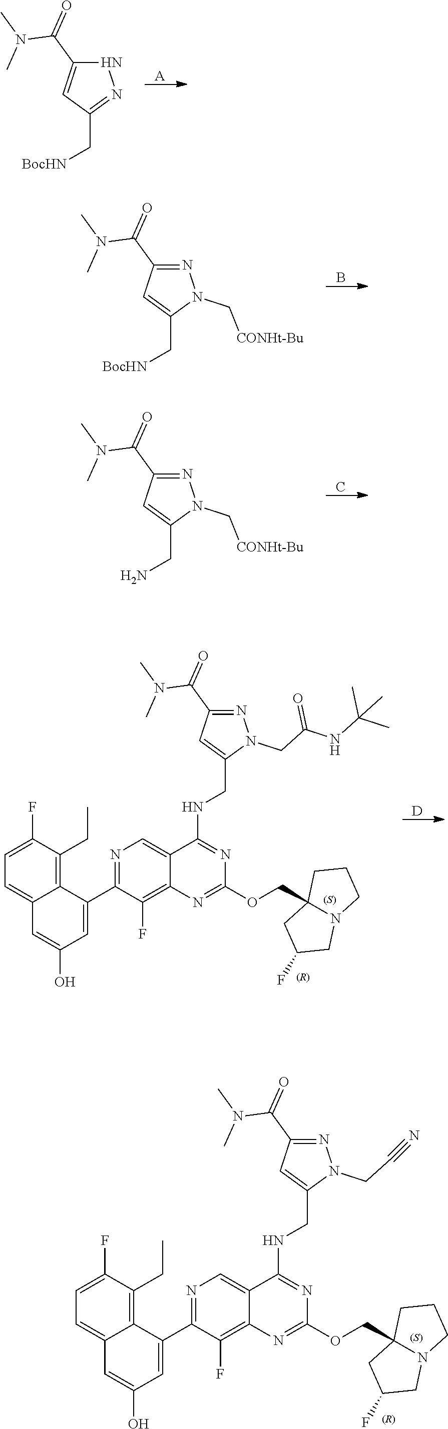

Step A. tert-butyl N-[[2-[2-(tert-butylamino)-2-oxo-ethyl]-5-(dimethylcarbamoyl) pyrazol-3-yl]methyl]carbamate: To a solution of tert-butyl N-[[5-(dimethylcarbamoyl)-1H-pyrazol-3-yl]methyl]carbamate (650 mg, 1.0 equiv) and 2-bromo-N-tert-butyl-acetamide (940 mg, 2 equiv) in DMF (7 mL) was added Cs₂CO₃ (2.4 g, 3.0 equiv) at 25° C. The mixture was stirred at 80° C. for 12 hours before being poured into ice- water (20 mL) and stirred for 2 mins. The aqueous phase was extracted with ethyl acetate (20 mL×3). The combined organic phases were washed with brine (30 mL), dried with anhydrous Na₂SO₄, filtered and concentrated under vacuum. The residue was purified by prep-HPLC [C18, 0.1% formic acid condition], which was further separated by SFC (column: DAICEL CHIRALPAK AD (250 mm×30 mm 10 um); mobile phase: [0.1% NH₃·H₂O IPA]; B %: 35%-35%, 2.3 min) to afford the title compound (369 mg, 39.5% yield) as a yellow solid. ¹HNMR (400 MHZ, METHANOL-d₄) δ 1.35 (s, 9H) 1.44 (s, 9H) 3.07 (s, 3H) 3.30 (br s, 3H) 4.28 (s, 2H) 4.86 (br s, 2H) 6.53 (s, 1H).

Step B. 5-(aminomethyl)-1-[2-(tert-butylamino)-2-oxo-ethyl]-N,N-dimethyl-pyrazole-3-carboxamide: To a solution of tert-butyl N-[[2-[2-(tert-butylamino)-2-oxo-ethyl]-5-(dimethylcarbamoyl) pyrazol-3-yl]methyl]carbamate (341 mg, 1.0 equiv) in dioxane (5 mL) was added HCl/dioxane (4 M, 223 μL, 1.0 equiv) at 25° C. The mixture was stirred at 25° C. for 1 hour. The mixture was directly concentrated under vacuum to afford the title compound (270 mg, crude, HCl) as a white solid.

Step C. 1-(2-(tert-butylamino)-2-oxoethyl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (100 mg, 1.0 equiv) and 5-(aminomethyl)-1-[2-(tert-butylamino)-2-oxo-ethyl]-N,N-dimethyl-pyrazole-3-carboxamide (134 mg, 2.5 equiv, HCl) in DMF (1.5 mL) was added K₃PO₄ (179 mg, 5.0 equiv) at 25° C. The mixture was stirred at 60° C. for 12 hours. The residue was poured into ice-water (20 mL) and stirred for 2 mins. The aqueous phase was extracted with ethyl acetate (20 mL×3). The combined organic phases were washed with brine (30 mL), dried with anhydrous Na₂SO₄, filtered and concentrated under vacuum. The residue was purified by prep-HPLC [C18, 0.1% formic acid condition] to afford the title compound (78 mg, 59% yield) as white solid. LCMS (ESI, M+1): m/z=774.4.

Step D. 1-(cyanomethyl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide: To a solution of 1-(2-(tert-butylamino)-2-oxoethyl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide (28 mg, 1.0 equiv) in DMF (0.5 mL) was added POCl₃ (6.8 mg, 1.23 equiv) at 25° C. The mixture was stirred at 55° C. for 2 hours. The residue was purified by prep-HPLC [C18, 0.1% NH₄HCO₃ condition] to afford the title compound (4.8 mg, 18.8% yield) as a white solid. ¹H NMR (400 MHZ, METHANOL-d₄) δ 0.76 (t, J=7.38 Hz, 3H) 1.84-2.05 (m, 3H) 2.07-2.49 (m, 5H) 2.97-3.07 (m, 1H) 3.10 (s, 3H) 3.36 (s, 3H) 4.28-4.36 (m, 2H) 4.59 (br s, 2H) 4.94-5.10 (m, 3H) 5.22-5.41 (m, 1H) 5.63 (s, 2H) 6.80 (s, 1H) 7.04 (d, J=2.50 Hz, 1H) 7.24 (t, J=9.2 Hz, 1H) 7.30 (d, J=2.4 Hz, 1H) 7.67 (dd, J=9.2, 5.82 Hz, 1H) 9.17 (s, 1H); LCMS (ESI, M+1): m/z=700.3.

### Example 658

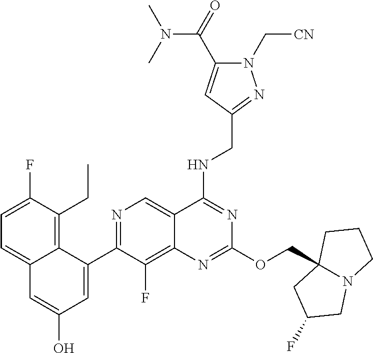 [SMILES: CCc1c(F)ccc2cc(O)cc(-c3ncc4c(NCc5cc(C(=O)N(C)C)n(CC#N)n5)nc(OC[C@@]56CCCN5C[C@H](F)C6)nc4c3F)c12]

### 1-(cyanomethyl)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-5-carboxamide

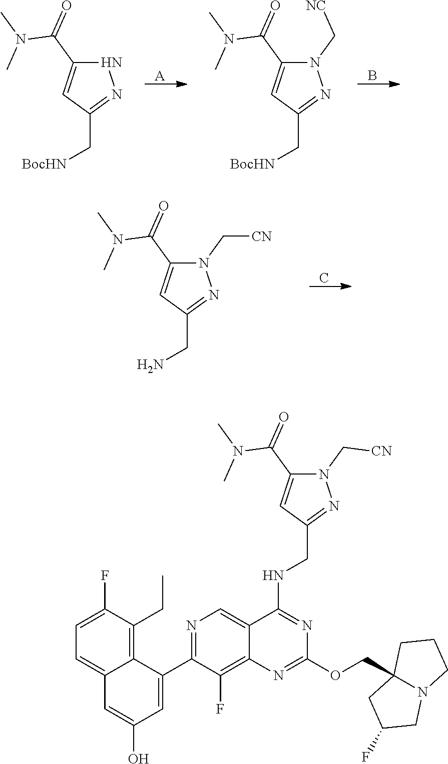

Step A. tert-butyl N-[[1-(cyanomethyl)-5-(dimethylcarbamoyl) pyrazol-3-yl]methyl]carbamate: To a solution of tert-butyl N-[[5-(dimethylcarbamoyl)-1H-pyrazol-3-yl]methyl]carbamate (150 mg, 1.0 equiv) in DMF (5 mL) was added Cs₂CO₃ (546 mg, 3.0 equiv) and 2-bromoacetonitrile (134 mg, 2.0 equiv). The mixture was stirred at 25° C. for 2 hours. The reaction mixture was filtered, concentrated and purified by reversed phase flash chromatography (C18, 0.1% NH₃·H₂O in water, 10-50% ACN) to afford the title compound (70.0 mg 33% yield) as a brown oil; ¹H NMR (400 MHZ, DMSO-d₆) δ=7.39-7.25 (m, 1H), 6.49 (s, 1H), 5.39 (s, 2H), 4.10 (br d, J=5.0 Hz, 2H), 3.08 (s, 3H), 2.99 (s, 3H), 1.40-1.36 (m, 9H)

Step B. 5-(aminomethyl)-2-(cyanomethyl)-N,N-dimethyl-pyrazole-3-carboxamide: To a mixture of tert-butyl N-[[1-(cyanomethyl)-5-(dimethylcarbamoyl) pyrazol-3-yl]methyl]carbamate (45.0 mg, 90.2% purity, 1.0 equiv) in DCM (0.2 mL) was added dropwise TFA (0.1 mL, 10.2 equiv) in DCM (0.1 mL). The mixture was stirred at 0° C. for 1 hour. The reaction mixture was poured into water (2 mL) and pH was adjusted to ˜8 by NH₃·H₂O. The mixture was concentrated and purified with prep-HPLC [Waters Xbridge 150×25 mm×5 um; A: water (NH₄HCO₃), B: ACN, B %: 1%-20% over 10 min] to afford the title compound (12.0 mg, 44% yield) as a yellow oil; ¹H NMR (400 MHZ, METHANOL-d₄) δ=6.69 (s, 1H), 5.40-5.37 (m, 2H), 3.92 (s, 2H), 3.23 (s, 3H), 3.13 (br s, 3H)

Step C. 1-(cyanomethyl)-3-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-5-carboxamide: The title compound was synthesized according to the procedure described for example 622 to produce the desired compound as a yellow gum; ¹H NMR (400 MHZ, DMSO-d₆) δ=10.01-9.83 (m, 1H), 9.61-9.49 (m, 1H), 9.33 (s, 1H), 7.76 (dd, J=6.4, 8.8 Hz, 1H), 7.39-7.31 (m, 2H), 6.99 (s, 1H), 6.75 (s, 1H), 5.45 (s, 2H), 5.36-5.17 (m, 1H), 4.88-4.70 (m, 2H), 4.18-4.01 (m, 2H), 3.10 (s, 3H), 3.00 (s, 3H), 2.97-2.71 (m, 2H), 2.17-1.97 (m, 5H), 1.87-1.73 (m, 3H), 0.71 (br t, J=7.3 Hz, 3H); LCMS (ESI, M+1): m/z=700.5.

### Example 659

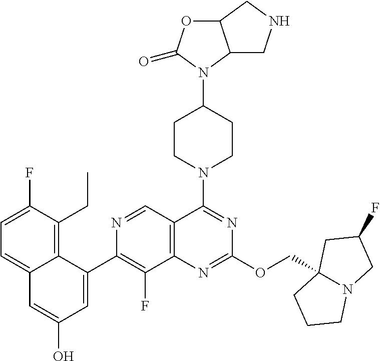 [SMILES: CCc1c(F)ccc2cc(O)cc(-c3ncc4c(N5CCC(N6C(=O)OC7CNCC76)CC5)nc(OC[C@@]56CCCN5C[C@H](F)C6)nc4c3F)c12]

### 3-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperidin-4-yl)hexahydro-2H-pyrrolo[3,4-d]oxazol-2-one

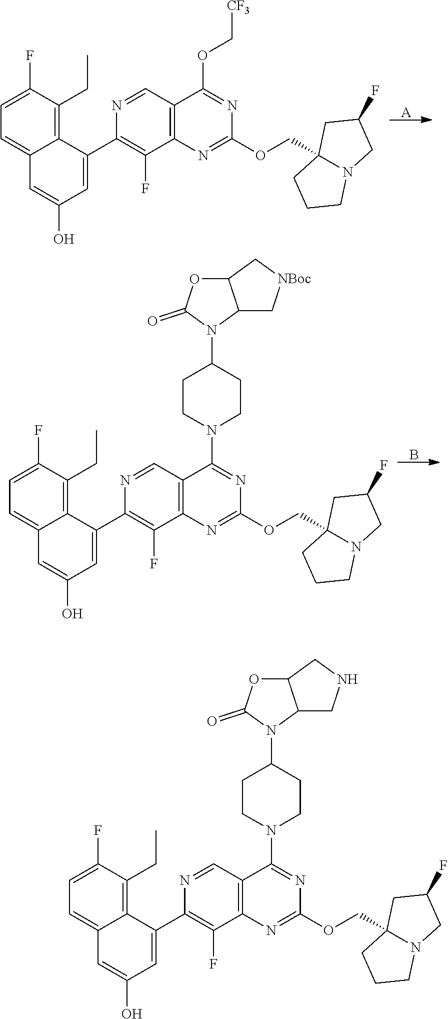

Step A. tert-butyl 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-4-yl)-2-oxotetrahydro-2H-pyrrolo[3,4-d]oxazole-5 (3H)-carboxylate: A mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (142 mg, 1.0 equiv), tert-butyl N-[[4-methyl-2-oxo-3-(4-piperidyl) oxazolidin-5-yl]methyl]carbamate (92.2 mg, 1.1 equiv, HCl), K₃PO₄ (50.9 mg, 1.0 equiv) in DMF (1 mL) was degassed and purged with N₂ for 3 times, and then the mixture was stirred at 25° C. for 12 hours under N₂ atmosphere. The mixture was filtered and concentrated and purified with prep-HPLC [C18, 0.1% formic acid condition] to afford the title compound (72 mg, 37% yield). ¹H NMR (400 MHZ, CHLOROFORM-d) δ=8.98-8.84 (m, 1H), 7.59-7.52 (m, 1H), 7.24-7.12 (m, 2H), 6.97-6.63 (m, 1H), 5.49-5.24 (m, 1H), 4.98-4.80 (m, 1H), 4.75-4.45 (m, 3H), 4.39-4.31 (m, 1H), 4.19-3.96 (m, 1H), 3.94-3.82 (m, 2H), 3.68-3.35 (m, 5H), 3.33-3.01 (m, 4H), 2.54-1.78 (m, 11H), 1.56-1.39 (m, 9H), 0.87-0.70 (m, 3H).

Step B. 3-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperidin-4-yl)hexahydro-2H-pyrrolo[3,4-d]oxazol-2-one: A mixture of tert-butyl 3-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-4-yl)-2-oxotetrahydro-2H-pyrrolo[3,4-d]oxazole-5 (3H)-carboxylate (20.1 mg, 1.0 equiv) in HCl/dioxane (0.5 mL) and MeOH (0.5 mL) stirred at 25° C. for 1 hour under N₂ atmosphere. The mixture was concentrated and purified with prep-HPLC [C18, 0.1% formic acid condition] to afford the title compound (16 mg, 86% yield, hydrochloride). ¹H NMR (400 MHZ, METHANOL-d₄) δ=9.13 (s, 1H), 8.44 (br s, 1H), 7.71 (dd, J=6.0, 9.0 Hz, 1H), 7.34 (d, J=2.8 Hz, 1H), 7.28 (t, J=9.6 Hz, 1H), 7.08 (s, 1H), 5.65-5.42 (m, 1H), 5.13 (br dd, J=4.8, 6.9 Hz, 1H), 4.86 (br s, 1H), 4.70-4.51 (m, 3H), 4.02-3.34 (m, 10H), 3.13-2.90 (m, 2H), 2.70-2.02 (m, 12H), 0.82 (t, J=7.2 Hz, 3H); ¹⁹F NMR (377 MHz, METHANOL-d4) δ=−121.07 (br s, 1F), −139.19 (br s, 1F), −173.99 (br s, 1F); LCMS (ESI, M+1): m/z=704.3.

### Example 660

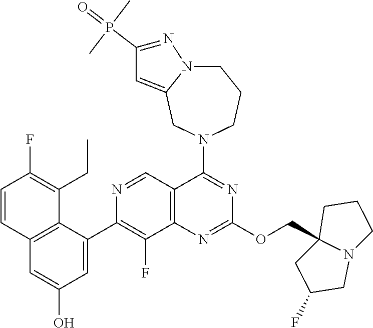 [SMILES: CCc1c(F)ccc2cc(O)cc(-c3ncc4c(N5CCCn6nc(P(C)(C)=O)cc6C5)nc(OC[C@@]56CCCN5C[C@H](F)C6)nc4c3F)c12]

### (5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-5,6,7,8-tetrahydro-4H-pyrazolo[1,5-a][1,4]diazepin-2-yl)dimethylphosphine oxide

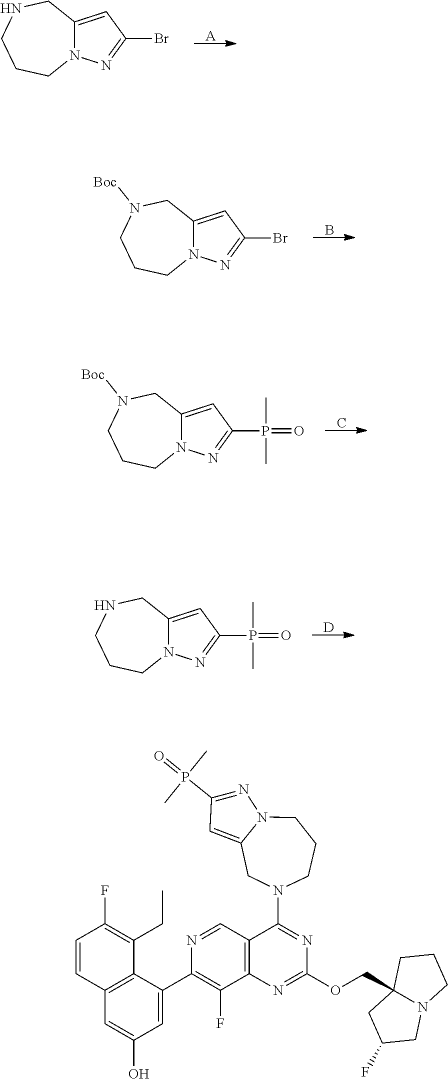

Step A. tert-butyl 2-bromo-7,8-dihydro-4H-pyrazolo[1,5-a][1,4]diazepine-5(6H)-carboxylate: To a solution of 2-bromo-5,6,7,8-tetrahydro-4H-pyrazolo[1,5-a][1,4]diazepine (650 mg, 1.0 equiv, HCl) in DCM (8 mL) was added TEA (520 mg, 716 μL, 2.0 equiv) and Boc₂O (842 mg, 1.5 equiv). The mixture was stirred at 25° C. for 2 hours. The reaction mixture was concentrated under reduced pressure to remove DCM. The residue was purified by flash silica gel chromatography (ISCOR; 12 g SepaFlash® Silica Flash Column, Eluent of 0˜30% Ethyl acetate/Petroleum ethergradient @ 30 mL/min) to afford the title compound (250 mg, 28.9% yield, 94.0% purity) as a white solid. ¹H NMR (400 MHZ, CHLOROFORM-d) δ=6.32-6.12 (m, 1H), 4.48-4.35 (m, 4H), 3.78-3.65 (m, 2H), 1.91 (br d, J=4.4 Hz, 2H), 1.51-1.40 (m, 9H); LCMS (ESI, M+1): m/z=318.0.

Step B. tert-butyl 2-(dimethylphosphoryl)-7,8-dihydro-4H-pyrazolo[1,5-a][1,4]diazepine-5(6H)-carboxylate: A mixture of tert-butyl 2-bromo-7,8-dihydro-4H-pyrazolo[1,5-a][1,4]diazepine-5(6H)-carboxylate (250 mg, 1.0 equiv), methylphosphonoylmethane (74.1 mg, 1.2 equiv), Pd(OAc)₂ (17.7 mg, 0.1 equiv), K₃PO₄ (201 mg, 1.2 equiv) and (5-diphenylphosphanyl-9,9-dimethyl-xanthen-4-yl)-diphenyl-phosphane (45.7 mg, 1.0 equiv) in DMF (4.0 mL) was degassed and purged with N₂ for 3 times, and then the mixture was stirred at 140° C. for 12 hours under N₂ atmosphere. The mixture was cooled to 20° C. and filtered through a pad of celite. The filter cake was washed with EtOAc and the filtrate was evaporated and purified with prep-HPLC [C18, 0.1% formic acid condition] to afford the title compound (120 mg, 48.4% yield) as a yellow solid. LCMS (ESI, M−1): m/z=314.2.

Step C. dimethyl(5,6,7,8-tetrahydro-4H-pyrazolo[1,5-a][1,4]diazepin-2-yl)phosphine oxide: To tert-butyl 2-(dimethylphosphoryl)-7,8-dihydro-4H-pyrazolo[1,5-a][1,4]diazepine-5(6H)-carboxylate (120 mg, 1.0 equiv) in dioxane (1.0 mL) was added HCl/dioxane (4 M, 2.4 mL, 25.1 equiv). The mixture was stirred at 20° C. for 2 hours. The reaction mixture was concentrated under reduced pressure to afford the title compound (90 mg, 94.1% yield, HCl) as a white solid. ¹H NMR (400 MHZ, METHANOL-d₄) δ=6.94 (s, 1H), 4.69-4.63 (m, 2H), 4.58 (s, 2H), 3.68 (s, 3H), 3.62-3.58 (m, 2H), 2.23-2.15 (m, 2H), 1.82 (dd, J=1.6, 14.0 Hz, 6H).

Step D. (5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-5,6,7,8-tetrahydro-4H-pyrazolo[1,5-a][1,4]diazepin-2-yl)dimethylphosphine oxide: A mixture of 2-dimethylphosphoryl-5,6,7,8-tetrahydro-4H-pyrazolo[1,5-a][1,4]diazepine (30 mg, 1.0 equiv), 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (75.0 mg, 0.9 equiv), K₃PO₄ (89.6 mg, 3.0 equiv), 4 Å molecular sieves (10 mg) in DMF (0.2 mL) and MeCN (0.2 mL) was degassed and purged with N₂ for 3 times, and then the mixture was stirred at 60° C. for 12 hours under N₂ atmosphere. K₃PO₄, 4 Å molecular sieves was filtered. The filtrate was concentrated and purified by prep-HPLC [C18, 0.1% formic acid condition] to afford the title compound (10 mg, 8.9% yield, 88% purity) as a white solid (0.09 formic acid salt). ¹H NMR (400 MHZ, METHANOL-d₄) δ=9.17 (s, 1H), 8.57-8.51 (m, 1H), 7.68 (dd, J=6.0, 9.2 Hz, 1H), 7.31 (d, J=2.4 Hz, 1H), 7.26 (t, J=9.2 Hz, 1H), 7.05 (d, J=2.4 Hz, 1H), 6.91 (s, 1H), 5.46-5.37 (m, 1H), 5.36-5.27 (m, 2H), 4.63 (br d, J=5.6 Hz, 2H), 4.45 (br d, J=4.4 Hz, 2H), 4.40-4.30 (m, 2H), 3.37 (br s, 1H), 3.16-3.06 (m, 1H), 2.54-2.37 (m, 4H), 2.36-2.26 (m, 2H), 2.25-2.03 (m, 5H), 2.00-1.93 (m, 1H), 1.81 (s, 3H), 1.78 (s, 3H), 1.38-1.24 (m, 1H), 0.79 (t, J=6.8 Hz, 3H); LCMS (ESI, M+1): m/z=706.2.

### Example 661

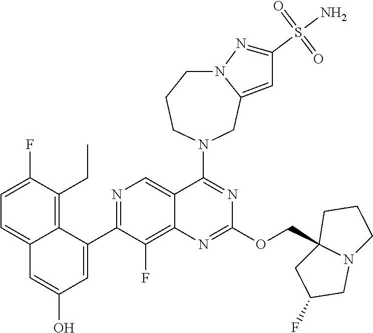 [SMILES: CCc1c(F)ccc2cc(O)cc(-c3ncc4c(N5CCCn6nc(S(N)(=O)=O)cc6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-5,6,7,8-tetrahydro-4H-pyrazolo[1,5-a][1,4]diazepine-2-sulfonamide

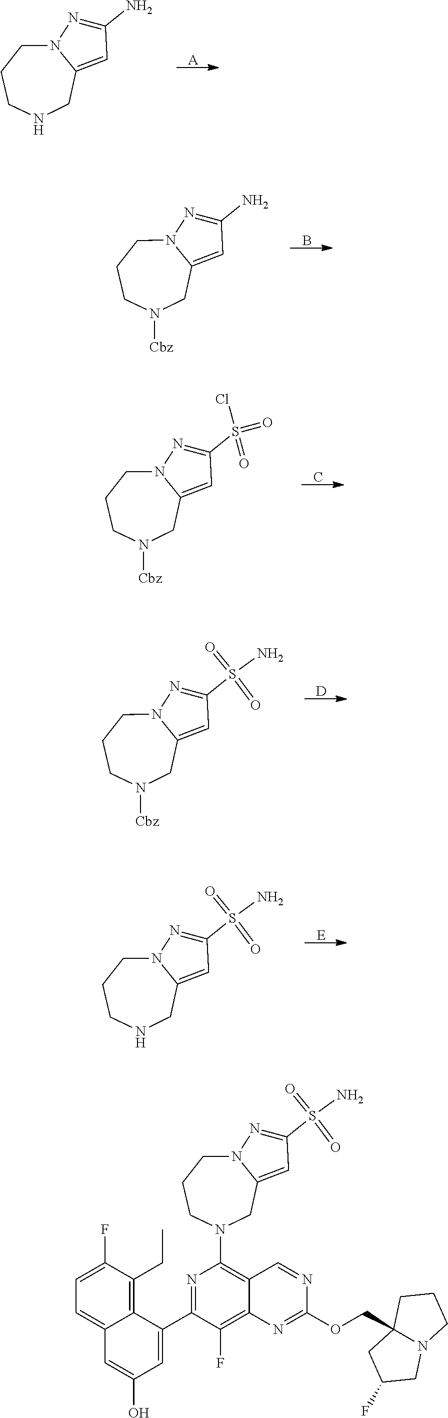

Step A. benzyl 2-amino-7,8-dihydro-4H-pyrazolo[1,5-a][1,4]diazepine-5(6H)-carboxylate: To a mixture of 5,6,7,8-tetrahydro-4H-pyrazolo[1,5-a][1,4]diazepin-2-amine (500 mg, 1.0 equiv, HCl), TEA (1.07 g, 4.0 equiv) in THF (10 mL) was added CbzCl (452 mg, 1.0 equiv) at 0° C. The reaction was stirred at 20° C. for 2 hours. The mixture was diluted with water (10 mL) and extracted with EtOAc (3×10 mL). The combined organic layers were washed with brine, dried over anhydrous sodium sulfate, concentrated, and purified by reversed phase flash chromatography [C18, 0.1% formic acid condition] to afford the title compound (450 mg, 56% yield) as a yellow oil; ¹H NMR (400 MHZ, chloroform-d) δ=7.39-7.29 (m, 5H), 5.67-5.45 (m, 1H), 5.13-5.09 (m, 2H), 4.46-4.39 (m, 2H), 4.23-4.18 (m, 2H), 3.77-3.71 (m, 2H), 1.97-1.85 (m, 2H); LCMS (ESI, M+1): m/z=286.7.

Step B. benzyl 2-(chlorosulfonyl)-7,8-dihydro-4H-pyrazolo[1,5-a][1,4]diazepine-5(6H)-carboxylate: To a mixture of benzyl 2-amino-7,8-dihydro-4H-pyrazolo[1,5-a][1,4]diazepine-5(6H)-carboxylate (500 mg, 1.0 equiv) in ACN (40 mL) at 0° C. was added a solution of HCl (2.55 g, 37% purity, 14.8 equiv) in H₂O (0.9 mL) followed by aqueous solution of NaNO₂ (241 mg, 2.0 equiv) in H₂O (0.8 mL). After stirring at 0° C. for 45 minutes, the mixture was treated with AcOH (945 mg, 9.0 equiv), CuCl (86.4 mg, 0.5 equiv) and CuCl₂ (141 mg, 0.6 equiv) and purged with SO₂ gas at 0° C. for 25 minutes. The reaction was stirred at 0-15° C. for 12 hours. The mixture was poured into ice-water (10 mL) and extracted with EtOAc (3×15 mL). The combined organic layers were washed with brine, dried over anhydrous sodium sulfate, concentrated to afford the title compound (600 mg, crude) as a dark green oil.

Step C. benzyl 2-sulfamoyl-7,8-dihydro-4H-pyrazolo[1,5-a][1,4]diazepine-5(6H)-carboxylate: A mixture of benzyl 2-(chlorosulfonyl)-7,8-dihydro-4H-pyrazolo[1,5-a][1,4]diazepine-5(6H)-carboxylate (400 mg, 1 equiv) in NH₃·THF (9 mL, 20% purity, 1.0 equiv) was stirred at 20° C. for 2 hours. The mixture was concentrated, diluted with saturated NH₄Cl aqueous solution (5 mL), extracted with EtOAc (3×10 mL), concentrated, and purified by reversed phase flash chromatography [C18, 0.1% formic acid condition] to afford the title compound (90 mg, 23% yield) as a yellow oil; ¹H NMR (400 MHZ, chloroform-d) δ=7.42-7.29 (m, 5H), 6.72-6.51 (m, 1H), 5.17-5.07 (m, 2H), 4.97-4.88 (m, 2H), 4.60-4.51 (m, 2H), 4.51-4.46 (m, 2H), 3.86-3.77 (m, 2H), 2.04-1.92 (m, 2H); LCMS (ESI, M+1): m/z=350.8.

Step D. 5,6,7,8-tetrahydro-4H-pyrazolo[1,5-a][1,4]diazepine-2-sulfonamide; To a solution of benzyl 2-sulfamoyl-7,8-dihydro-4H-pyrazolo[1,5-a][1,4]diazepine-5(6H)-carboxylate (80 mg, 1.0 equiv) in MeOH (2 mL) was added NH₃·MeOH (4 M, 0.5 mL, 1.0 equiv) and Pd(OH)₂ (30 mg, 10%) under nitrogen. The reaction was degassed and purged with hydrogen 3 times. The reaction was stirred at 20° C. for 1 hours under hydrogen (15 psi) atmosphere. The mixture was filtered and concentrated to afford the title compound (35 mg, crude) as yellow oil.

Step E. 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-5,6,7,8-tetrahydro-4H-pyrazolo[1,5-a][1,4]diazepine-2-sulfonamide: To a mixture of 5,6,7,8-tetrahydro-4H-pyrazolo[1,5-a][1,4]diazepine-2-sulfonamide (29 mg, 2.0 equiv) and 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (40 mg, 1.0 equiv) in DMF (0.5 mL) and ACN (0.5 mL) were added 4 Å molecular sieves (20 mg) and K₃PO₄ (43 mg, 3.0 equiv). The reaction mixture was stirred at 40° C. for 36 hours. The mixture was filtered and purified by prep-HPLC [Waters xbridge 150×25 mm×10 um; mobile phase: A: water (NH₄HCO₃), B: ACN; B %: 20%-50% over 8 min] and followed by prep-HPLC [Waters xbridge 150×25 mm×10 μm; mobile phase: A: water (FA), B: CAN; B %: 8%-38% over 8 min] to afford the title compound (6.89 mg, 13.6% yield, 0.38 FA) as a white solid (0.38 formic acid salt); SFC: >99% ee, Chiralcel OD-3 50×4.6 mm I.D., 3 μm; Isocratic elution: 40% MeOH+ACN (0.05% DEA) in CO₂, 3 mL/min; 220 nm; t_{R}: 0.604 min; ¹H NMR (400 MHZ, dimethylsulfoxide-d₆) δ=9.91-9.85 (m, 1H), 9.07-9.03 (m, 1H), 7.78-7.71 (m, 1H), 7.39-7.23 (m, 2H), 6.99-6.92 (m, 1H), 6.65-6.57 (m, 1H), 5.52-5.32 (m, 1H), 4.44-4.34 (m, 2H), 4.27-4.10 (m, 2H), 4.09-4.00 (m, 2H), 3.82-3.66 (m, 2H), 3.19-3.15 (m, 3H), 3.10-3.04 (m, 2H), 2.31-2.27 (m, 1H), 2.24-2.19 (m, 1H), 2.18-2.06 (m, 2H), 2.03-1.91 (m, 2H), 1.90-1.75 (m, 3H), 0.75-0.63 (m, 3H); ¹H NMR (400 MHZ, dimethylsulfoxide-d₆+deuterium oxide-d₂) δ=9.10-9.03 (m, 1H), 7.75-7.68 (m, 1H), 7.37-7.31 (m, 1H), 7.30-7.25 (m, 1H), 6.97-6.92 (m, 1H), 6.65-6.59 (m, 1H), 5.57-5.34 (m, 1H), 4.40-4.35 (m, 2H), 4.35-4.13 (m, 2H), 4.05-3.97 (m, 2H), 3.77-3.65 (m, 2H), 3.61-3.45 (m, 2H), 3.18-3.08 (m, 3H), 2.34-2.30 (m, 1H), 2.29-2.15 (m, 2H), 2.12-1.99 (m, 3H), 1.88-1.74 (m, 3H), 0.71-0.63 (m, 3H); ¹⁹F NMR (400 MHZ, dimethylsulfoxide-d₆) δ=−119.825, −141.484, −172.667; LCMS (ESI, M+1): m/z=709.3.

### Example 662

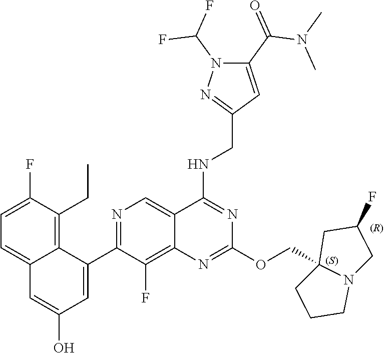

### 1-(difluoromethyl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide

### Example 663

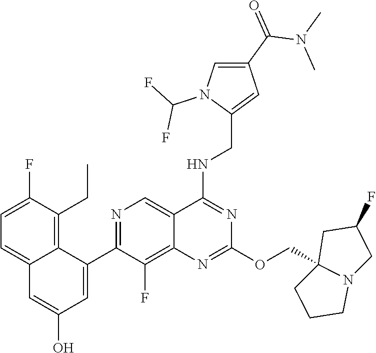 [SMILES: CCc1c(F)ccc2cc(O)cc(-c3ncc4c(NCc5cc(C(=O)N(C)C)cn5C(F)F)nc(OC[C@@]56CCCN5C[C@H](F)C6)nc4c3F)c12]

### 1-(difluoromethyl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide

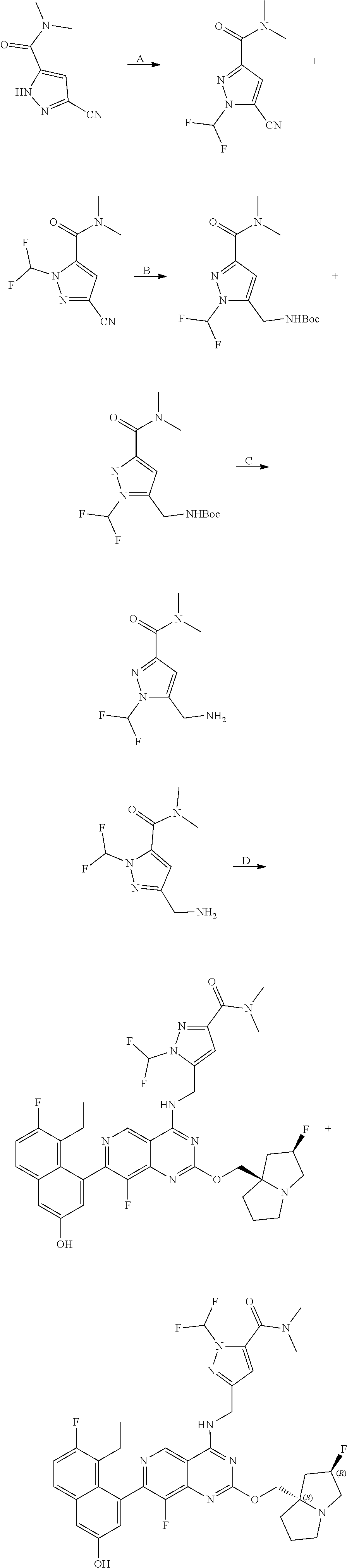

Step A. 5-cyano-1-(difluoromethyl)-N,N-dimethyl-1H-pyrazole-3-carboxamide: A mixture of 1-[[bromo(difluoro)methyl]-ethoxy-phosphoryl]oxyethane (195 mg, 1.0 equiv), 3-cyano-N,N-dimethyl-1H-pyrazole-5-carboxamide (120 mg, 1.0 equiv), potassium fluoride (84.9 mg, 2.0 equiv) in acetonitrile (5.0 mL) was degassed and purged with nitrogen for 3 times, and stirred at 20° C. for 12 hours under nitrogen atmosphere. The reaction mixture was partitioned between water (5.00 mL) and ethyl acetate (5.00 mL). The organic phase was separated, dried over anhydrous sodium sulfate filtered and concentrated under reduced pressure to afford the title compound, a mixture of regioisomers (95.0 mg, 73% yield) as a yellow solid; ¹H NMR (400 MHZ, CHLOROFORM-d) δ=3.14 (s, 3H) 3.33 (s, 3H) 7.42 (s, 1H) 7.44-7.76 (m, 1H)

Step B. tert-butyl ((1-(difluoromethyl)-3-(dimethylcarbamoyl)-1H-pyrazol-5-yl)methyl)carbamate: A mixture of 5-cyano-1-(difluoromethyl)-N,N-dimethyl-pyrazole-3-carboxamide (95.0 mg, 1.0 equiv), di-tert-butyl dicarbonate (193 mg, 2.0 equiv) and Palladium 10% on carbon (20.0 mg, 10% purity, 1.0 equiv) in methanol (10 mL) was degassed and purged with hydrogen (15.0 psi) for 3 times, and stirred at 20° C. for 14 hours under hydrogen (15.0 psi) atmosphere. The mixture was filtered and concentrated in vacuum to afford the title compound, a mixture of regioisomers (110 mg, 78% yield) as a yellow solid; LCMS (ESI, M+1): m/z=319.1

Step C. 5-(aminomethyl)-1-(difluoromethyl)-N,N-dimethyl-1H-pyrazole-3-carboxamide: To a solution of tert-butyl N-[[2-(difluoromethyl)-5-(dimethylcarbamoyl) pyrazol-3-yl]methyl]carbamate (100 mg, 1.0 equiv) in acetonitrile (1.0 mL) was added HCl/dioxane (4M, 1.0 mL). The mixture was stirred at 20° C. for 0.5 hour. The mixture was concentrated under vacuum. The residue was purified by prep-HPLC (neutral condition; column: Waters Xbridge 150×25 mm×5 um; mobile phase: [water (ammonia hydroxide v/v)-ACN]; B %: 1%-30%, 10 min) to afford the title compound, a mixture of regioisomers (10.0 mg, 15% yield) as a white solid.

Step D. 1-(difluoromethyl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide and 1-(difluoromethyl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide: To a solution of 5-(aminomethyl)-1-(difluoromethyl)-N,N-dimethyl-pyrazole-3-carboxamide (70.0 mg, 1.0 equiv) and 5-ethyl-6-fluoro-4-[8-fluoro-2-[[(2R,8S)-2-fluoro-1,2,3,5,6,7-hexahydropyrrolizin-8-yl]methoxy]-4-(2,2,2-trifluoroethoxy)pyrido[4,3-d]pyrimidin-7-yl]naphthalen-2-ol (190 mg, 1.0 equiv) in N,N-dimethylformamide (1.0 mL) and acetonitrile (1.0 mL) was added potassium phosphate (68.1 mg, 1.00 equiv). The mixture was stirred at 60° C. for 3 hours. The mixture was concentrated and purified with SFC separation [column: DAICEL CHIRALPAK AS (250 mm×30 mm, 10 μm); mobile phase: [0.1% NH₃H₂O EtOH]; B %: 30%-30%, 6 minutes] and prep-HPLC [column: Unisil 3-100 C18 Ultra 150×50 mm×3 μm; mobile phase: [water (FA)-ACN]; B %: 13%-43%, 7 minutes] to afford the title compound EXAMPLE 662 (3.96 mg, 1.7% yield, 0.4FA) and EXAMPLE 663 (3.82 mg, 1.6% yield, 0.4FA) as yellow solids.

EXAMPLE 662: ¹H NMR (400 MHZ, METHANOL-d₄) δ ppm 0.78 (br t, J=7.19 Hz, 3H) 1.86-2.04 (m, 2H) 2.04-2.12 (m, 2H) 2.13-2.20 (m, 2H) 2.21-2.37 (m, 2H) 2.37-2.52 (m, 2H) 3.09 (s, 3H) 3.32-3.34 (m, 3H) 3.41 (br d, J=10.8 Hz, 2H) 4.29-4.43 (m, 2H) 5.05-5.18 (m, 2H) 5.27-5.47 (m, 1H) 6.83 (s, 1H) 7.03-7.07 (m, 1H) 7.25 (t, J=9.4 Hz, 1H) 7.31 (d, J=2.4 Hz, 1H) 7.61-8.02 (m, 2H) 8.48-8.56 (m, 1H) 9.22 (s, 1H); LCMS (ESI, M+1): m/z=711.3.

EXAMPLE 663: ¹H NMR (400 MHZ, METHANOL-d₄) δ ppm 0.79 (br t, J=7.2 Hz, 3H) 1.87-2.55 (m, 10H) 3.10 (s, 3H) 3.12 (s, 3H) 3.36-3.44 (m, 2H) 4.26-4.44 (m, 2H) 4.91-4.98 (m, 2H) 5.30 (br s, 1H) 5.26-5.47 (m, 1H) 5.43 (br s, 1H) 6.79 (s, 1H) 7.00-7.09 (m, 1H) 7.25 (br t, J=9.6 Hz, 1H) 7.31 (d, J=2.4 Hz, 1H) 7.61 (br t, J=5.6 Hz, 1H) 7.68 (br dd, J=8.88, 5.88 Hz, 1H) 8.49-8.56 (m, 1H) 9.20 (s, 1H); LCMS (ESI, M+1): m/z=711.3.

### Example 664

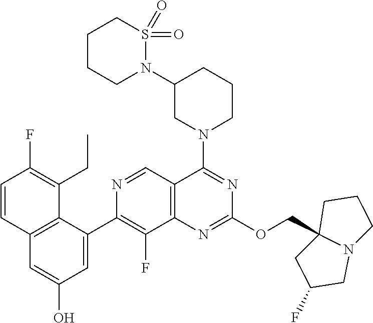 [SMILES: CCc1c(F)ccc2cc(O)cc(-c3ncc4c(N5CCCC(N6CCCCS6(=O)=O)C5)nc(OC[C@@]56CCCN5C[C@H](F)C6)nc4c3F)c12]

### 2-(1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) piperidin-3-yl)-1,2-thiazinane 1,1-dioxide

The title compound was synthesized according to the procedure described for example 538 to produce the desired compound as a white solid (0.18 formic acid salt). ¹H NMR (400 MHZ, METHANOL-d₄) δ=9.09 (s, 1H), 8.54 (s, 1H), 7.68 (dd, J=6.0, 9.2 Hz, 1H), 7.30 (d, J=2.4 Hz, 1H), 7.25 (t, J=9.2 Hz, 1H), 7.06 (dd, J=2.4, 12.4 Hz, 1H), 5.49-5.25 (m, 1H), 4.64-4.47 (m, 2H), 4.40-4.16 (m, 2H), 3.54-3.34 (m, 6H), 3.16-3.02 (m, 3H), 2.63-1.53 (m, 18H), 0.85-0.72 (m, 3H). LCMS (ESI, M+1): m/z=711.3.

### Example 665

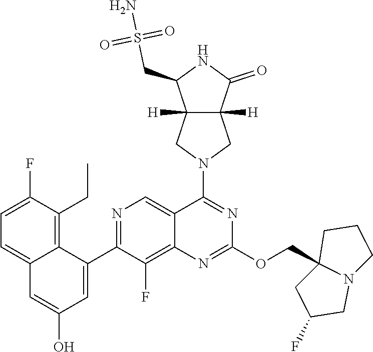 [SMILES: CCc1c(F)ccc2cc(O)cc(-c3ncc4c(N5C[C@@H]6[C@@H](CS(N)(=O)=O)NC(=O)[C@@H]6C5)nc(OC[C@@]56CCCN5C[C@H](F)C6)nc4c3F)c12]

### 1-((1S,3aS,6aR)-5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-oxooctahydropyrrolo[3,4-c]pyrrol-1-yl)methanesulfonamide

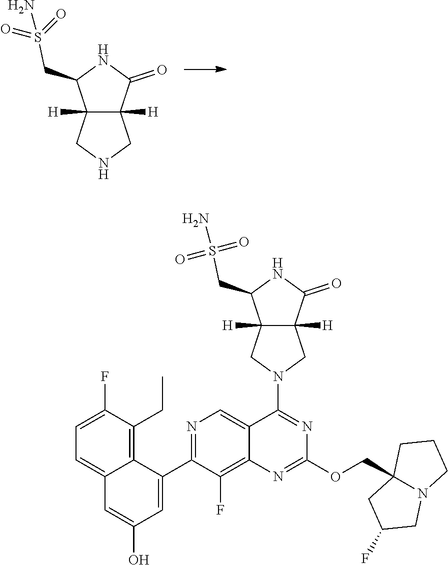 [SMILES: NS(=O)(=O)C[C@H]1NC(=O)[C@@H]2CNC[C@H]12 ; CCc1c(F)ccc2cc(O)cc(-c3ncc4c(N5C[C@@H]6[C@@H](CS(N)(=O)=O)NC(=O)[C@@H]6C5)nc(OC[C@@]56CCCN5C[C@H](F)C6)nc4c3F)c12]

To a solution of 1-((1S,3aS,6aR)-3-oxooctahydropyrrolo[3,4-c]pyrrol-1-yl)methanesulfonamide, Intermediate 13D (33.7 mg, 2.0 equiv, TFA) in DMF (1 mL) was added K₃PO₄ (53.7 mg, 5.0 equiv) and 4 Å molecular sieves (30.0 mg). The mixture was stirred at 25° C. for 0.5 hour. Then 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30.0 mg, 1.0 equiv) was added and the mixture was stirred at 25° C. for 1.5 hours. The resulting mixture was filtered and purified by prep-HPLC [Phenomenex Synergi Polar-RP 100×25 mm×4 um; A: water (TFA), B: ACN, B %: 26%-46% over 7 min] and then further re-purified by prep-HPLC [Waters Xbridge 150×25 mm×5 um; A: water (NH₄HCO₃), B: ACN, B %: 23%-53% over 8 min] and lyophilized to afford the title compound (7.34 mg, 20% yield) as a white solid; ¹H NMR (400 MHZ, DMSO-d₆) δ=9.97 (s, 1H), 9.36 (d, J=3.4 Hz, 1H), 8.09 (br s, 1H), 7.78 (dd, J=6.4, 9.0 Hz, 1H), 7.40-7.34 (m, 2H), 7.08 (s, 2H), 7.02 (d, J=2.8 Hz, 1H), 5.56-5.34 (m, 1H), 4.50-4.32 (m, 3H), 4.21 (br d, J=8.4 Hz, 2H), 3.96 (br d, J=6.8 Hz, 2H), 3.45 (br d, J=7.6 Hz, 4H), 3.28 (br s, 3H), 3.13-3.03 (m, 1H), 2.41-2.29 (m, 3H), 2.21-2.11 (m, 2H), 2.06-1.90 (m, 3H), 0.73 (d, J=7.2 Hz, 3H); LCMS (ESI, M+1): m/z=712.3. SFC: Rt=2.068 min; Method details: column: Chiralpak IC-3 50×4.6 mm I.D., 3 um; mobile phase: phase A for CO₂, and phase B for MeOH+ACN (0.05% DEA); gradient elution: 40% MeOH+ACN (0.05% DEA) in CO₂; flow rate: 3 mL/min; detector: PDA; column temp: 35° C.; back pressure: 100 Bar.

### Example 666

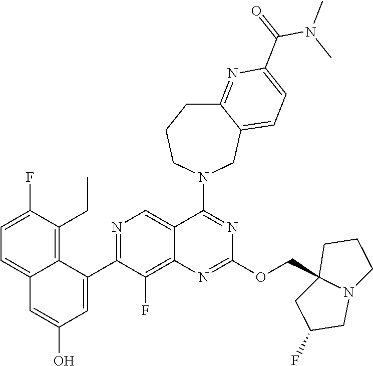 [SMILES: CCc1c(F)ccc2cc(O)cc(-c3ncc4c(N5CCCc6nc(C(=O)N(C)C)ccc6C5)nc(OC[C@@]56CCCN5C[C@H](F)C6)nc4c3F)c12]

### 6-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-6,7,8,9-tetrahydro-5H-pyrido[3,2-c]azepine-2-carboxamide

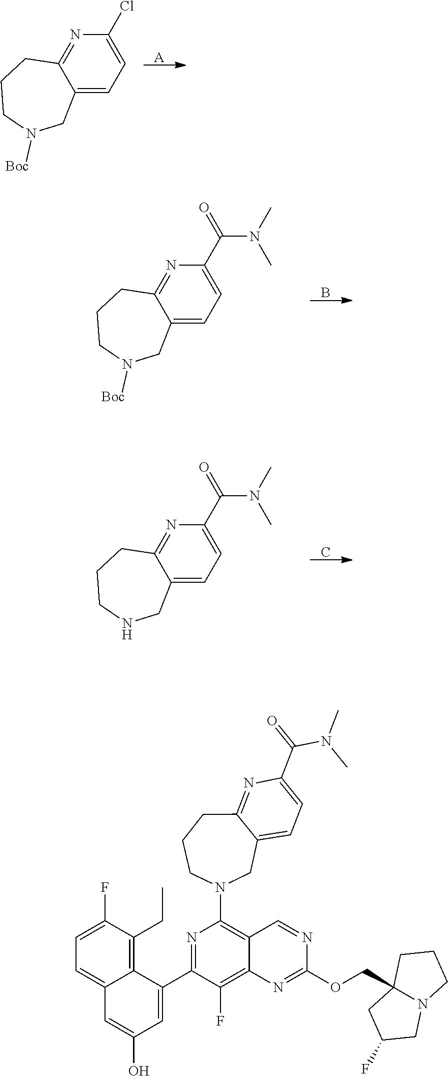

Step A. tert-butyl 2-(dimethylcarbamoyl)-8,9-dihydro-5H-pyrido[3,2-c]azepine-6(7H)-carboxylate: To a mixture of tert-butyl 2-chloro-8,9-dihydro-5H-pyrido[3,2-c]azepine-6(7H)-carboxylate (1.00 g, 1.0 equiv) and TEA (1.07 g, 3.0 equiv) in EtOH (10 mL) was added (1,1′-bis(diphenylphosphino)ferrocene)palladium (II) dichloride (258 mg, 0.1 equiv) and dimethylamine (478 mg, 3.0 equiv). The mixture was degassed and purged with argon for 3 times, and then the mixture was degassed and purged with CO for 3 times. The reaction was stirred at 80° C. for 24 hours under CO at 50 psi. The mixture was concentrated under vacuum to give a residue. The residue was diluted with water (15 mL) and extracted with ethyl acetate (3×30 mL). The combined organic layers were dried over anhydrous sodium sulfate, concentrated, and purified by reversed phase flash chromatography [C18, water (FA, 0.1%)/acetonitrile] to afford the title compound (700 mg, 62% yield) as a black oil. LCMS (ESI, M+1): m/z=320.2

Step B. N,N-dimethyl-6,7,8,9-tetrahydro-5H-pyrido[3,2-c]azepine-2-carboxamide: To a mixture of tert-butyl 2-(dimethylcarbamoyl)-8,9-dihydro-5H-pyrido[3,2-c]azepine-6(7H)-carboxylate (130 mg, 1.0 equiv) in MeCN (0.5 mL) was added HCl·dioxane (4 M, 0.6 mL). The reaction was stirred at 25° C. for 2 hours. The mixture was concentrated to afford the title compound (150 mg, crude, HCl) as a colorless oil and used into next step without further purification.

Step C. 6-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-6,7,8,9-tetrahydro-5H-pyrido[3,2-c]azepine-2-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (100 mg, 1.0 equiv), N,N-dimethyl-6,7,8,9-tetrahydro-5H-pyrido[3,2-c]azepine-2-carboxamide (74.0 mg, 1.7 equiv, HCl) in DMF (0.2 mL) and MeCN (0.2 mL) were added K₃PO₄ (179 mg, 5.0 equiv) and 4 Å molecular sieves (10 mg). The reaction was stirred at 40° C. for 12 hours. The mixture was filtered and purified by prep-HPLC (column: Phenomenex luna C18 150*25 mm*10 um; mobile phase: [water (FA)-ACN]; B %: 12%-42% over 10 min) to afford the title compound (52.0 mg, 42% yield, FA 0.26) as an off-white solid. ¹H NMR (400 MHZ, METHANOL-d₄) δ=9.15 (s, 1H), 8.05 (d, J=7.6 Hz, 1H), 7.68 (dd, J=6.0, 8.8 Hz, 1H), 7.45 (d, J=7.6 Hz, 1H), 7.33-7.19 (m, 2H), 7.04 (s, 1H), 5.47-5.29 (m, 1H), 5.29-5.18 (m, 2H), 4.49-4.36 (m, 2H), 4.34-4.16 (m, 2H), 3.50-3.33 (m, 3H), 3.26 (br d, J=7.2 Hz, 2H), 3.12 (s, 4H), 3.02 (s, 3H), 2.55-2.43 (m, 1H), 2.42-2.23 (m, 4H), 2.16 (br d, J=9.2 Hz, 2H), 2.11-2.02 (m, 2H), 2.01-1.88 (m, 1H), 0.78 (br t, J=7.2 Hz, 3H); LCMS (ESI, M+1): m/z=712.6.

### Example 667

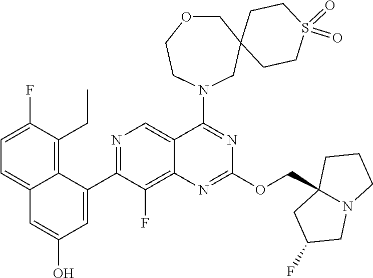 [SMILES: CCc1c(F)ccc2cc(O)cc(-c3ncc4c(N5CCOCC6(CCS(=O)(=O)CC6)C5)nc(OC[C@@]56CCCN5C[C@H](F)C6)nc4c3F)c12]

### 11-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-8-oxa-3-thia-11-azaspiro[5.6]dodecane 3,3-dioxide

The title compound was synthesized according to the procedure described for example 538 to produce the desired compound as a yellow solid (0.89 formic acid salt); ¹H NMR (400 MHZ, METHANOL-d4) δ=9.20 (s, 1H), 8.46 (br s, 1H), 7.69 (dd, J=5.6, 9.2 Hz, 1H), 7.31 (d, J=2.8 Hz, 1H), 7.26 (t, J=9.6 Hz, 1H), 7.04 (d, J=2.4 Hz, 1H), 5.63-5.36 (m, 1H), 4.72-4.37 (m, 4H), 4.37-4.23 (m, 3H), 4.21 (br d, J=2.8 Hz, 2H), 3.81-3.68 (m, 3H), 3.67-3.56 (m, 2H), 3.50-3.34 (m, 2H), 3.17-3.04 (m, 2H), 2.65-2.43 (m, 3H), 2.40-2.31 (m, 1H), 2.26-2.10 (m, 6H), 2.06-1.95 (m, 2H), 0.80 (t, J=7.6 Hz, 3H). LCMS (ESI, M+1): m/z=712.3

### Example 668

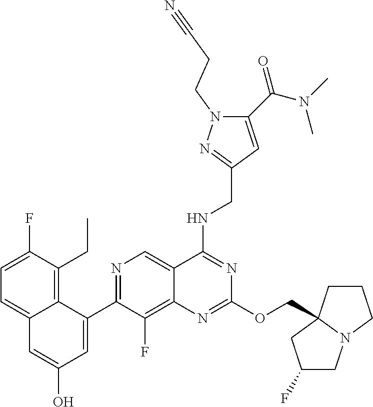 [SMILES: CCc1c(F)ccc2cc(O)cc(-c3ncc4c(NCc5cc(C(=O)N(C)C)n(CCC#N)n5)nc(OC[C@@]56CCCN5C[C@H](F)C6)nc4c3F)c12]

### 1-(2-cyanoethyl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide

### Example 669

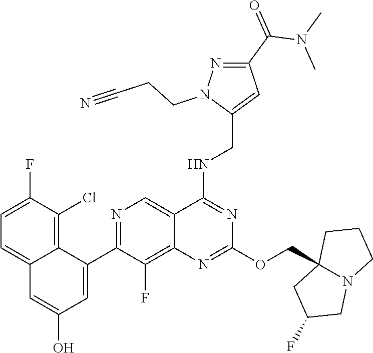 [SMILES: CN(C)C(=O)c1cc(CNc2nc(OC[C@@]34CCCN3C[C@H](F)C4)nc3c(F)c(-c4cc(O)cc5ccc(F)c(Cl)c45)ncc23)n(CCC#N)n1]

### 1-(2-cyanoethyl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide

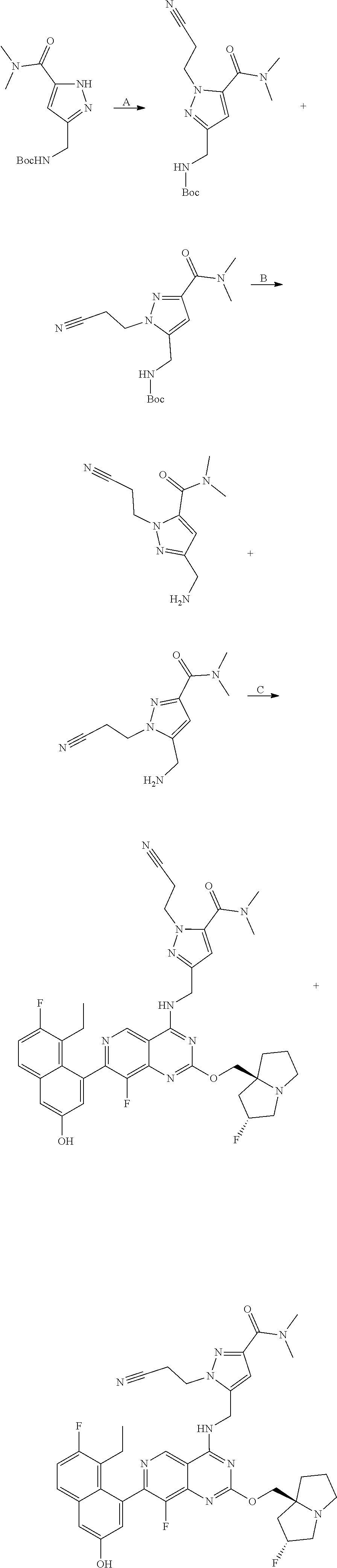

Step A. tert-butyl ((1-(2-cyanoethyl)-5-(dimethylcarbamoyl)-1H-pyrazol-3-yl)methyl)carbamate: To a mixture of tert-butyl ((5-(dimethylcarbamoyl)-1H-pyrazol-3-yl)methyl)carbamate (150 mg, 1.0 equiv) 3-bromopropanenitrile (149 mg, 2.0 equiv) in ACN (3 mL) was added Cs₂CO₃ (364 mg, 2.0 equiv). The reaction was stirred at 80° C. for 12 hours. The mixture was filtered and purified by prep-HPLC [Phenomenex luna C18 150×25 mm×10 μm; A: water (FA), B: ACN; B %: 20%-50% over 10 min] to afford the title compound, a mixture of regioisomers (60 mg, 33% yield) as a white oil; ¹H NMR (400 MHZ, chloroform-d) δ=6.39-6.31 (m, 1H), 5.05-4.89 (m, 1H), 4.61-4.53 (m, 2H), 4.35-4.29 (m, 2H), 3.20-3.09 (m, 6H), 2.98-2.93 (m, 2H), 1.47-1.45 (m, 9H); LCMS (ESI, M+1): m/z=322.2.

Step B. 3-(aminomethyl)-1-(2-cyanoethyl)-N,N-dimethyl-1H-pyrazole-5-carboxamide: To a mixture of tert-butyl ((1-(2-cyanoethyl)-5-(dimethylcarbamoyl)-1H-pyrazol-3-yl)methyl)carbamate (25 mg, 1.0 equiv) in DCM (43 μL) was added TFA (133 mg, 15 equiv). The reaction was stirred at 20° C. for 1 hour. The mixture was concentrated to afford the title compound, a mixture of regioisomers (26 mg, crude, TFA) as a yellow oil.

Step C. 1-(2-cyanoethyl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide and 1-(2-cyanoethyl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30 mg, 1.0 equiv) and 3-(aminomethyl)-1-(2-cyanoethyl)-N,N-dimethyl-1H-pyrazole-5-carboxamide (25 mg, 1.5 equiv, TFA) in ACN (0.2 mL) and DMF (0.2 mL) were added K₃PO₄ (32 mg, 3.0 equiv) and 4 Å molecular sieves (20 mg). The reaction was stirred at 40° C. for 12 hours. The mixture was filtered and purified by prep-HPLC [Phenomenex luna C18 150×25 mm×10 μm; A: water (FA), B: ACN; B %: 19%-49% over 10 min] followed by prep-HPLC [Phenomenex Luna C18 150×25 mm×10 μm; A: water (NH₄HCO₃), B: ACN; B %: 37%-67% over 8 min] to afford Example 668 (4.82 mg, 13% yield) as an off-white solid; SFC: Chiralpak AS-3 50×4.6 mm I.D., 3 μm; Isocratic elution: 20% MeOH (0.05% DEA) in CO₂, 3 mL/min; t_{R}: 1.224 min, 1.888 min; ¹H NMR (400 MHZ, dimethylsulfoxide-d₆) δ=9.95-9.89 (m, 1H), 9.57-9.49 (m, 1H), 9.35-9.31 (m, 1H), 7.79-7.73 (m, 1H), 7.39-7.31 (m, 2H), 7.00-6.97 (m, 1H), 6.64-6.61 (m, 1H), 5.36-5.17 (m, 1H), 4.87-4.70 (m, 2H), 4.50-4.40 (m, 2H), 4.16-4.10 (m, 1H), 4.08-4.01 (m, 1H), 3.11-3.04 (m, 5H), 3.03 (s, 2H), 3.01-2.95 (m, 4H), 2.87-2.76 (m, 1H), 2.37 (br s, 1H), 2.15-1.97 (m, 4H), 1.88-1.72 (m, 3H), 0.74-0.67 (m, 3H); ¹⁹F NMR (400 MHZ, dimethylsulfoxide-d₆) δ=−119.682, −139.503; −172.082; LCMS (ESI, M+1): m/z=714.4. and Example 669 (1.66 mg, 2.8% yield, 0.64FA) as a white solid; SFC: >99% ee, Chiralcel OD-3 50×4.6 mm I.D., 3 μm; Isocratic elution: 50% MeOH+ACN (0.05% DEA) in CO₂, 3 mL/min, 220 nm, t_{R}: 0.891 min; ¹H NMR (400 MHZ, methanol-d₄) δ=9.23-9.16 (m, 1H), 7.72-7.64 (m, 1H), 7.34-7.28 (m, 1H), 7.28-7.20 (m, 1H), 7.07-7.01 (m, 1H), 6.78-6.71 (m, 1H), 5.49-5.31 (m, 1H), 5.07-5.02 (m, 2H), 4.70-4.63 (m, 2H), 4.51-4.38 (m, 2H), 3.61-3.44 (m, 3H), 3.37 (s, 3H), 3.23-3.13 (m, 3H), 3.12-3.06 (m, 3H), 2.53-2.31 (m, 3H), 2.30-2.21 (m, 1H), 2.19-2.05 (m, 3H), 2.04-1.91 (m, 1H), 0.82-0.73 (m, 3H); ¹⁹F NMR (400 MHZ, methanol-d₄) δ=−121.153, −139.106, −173.885; LCMS (ESI, M+1): m/z=714.3.

### Example 670

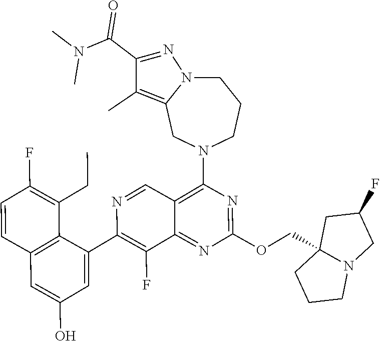 [SMILES: CCc1c(F)ccc2cc(O)cc(-c3ncc4c(N5CCCn6nc(C(=O)N(C)C)c(C)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N,3-trimethyl-5,6,7,8-tetrahydro-4H-pyrazolo[1,5-a][1,4]diazepine-2-carboxamide

The title compound was synthesized according to the procedure described for example 541 to produce the desired compound as a pink solid (0.14 formic acid salt); ¹H NMR (400 MHZ, METHANOL-d₄) δ=9.14-9.08 (m, 1H), 9.10 (s, 1H), 8.55-8.51 (m, 1H), 7.68 (dd, J=5.8, 9.0 Hz, 1H), 7.30 (d, J=2.6 Hz, 1H), 7.28-7.21 (m, 1H), 7.04 (d, J=2.4 Hz, 1H), 5.41 (br s, 1H), 5.26-5.12 (m, 2H), 4.50 (br d, J=9.4 Hz, 2H), 4.42 (br s, 2H), 4.38-4.28 (m, 2H), 3.44-3.32 (m, 4H), 3.30-3.27 (m, 1H), 3.17-3.06 (m, 7H), 2.52-2.27 (m, 5H), 2.23-1.89 (m, 6H), 0.78 (t, J=7.4 Hz, 3H); LCMS (ESI, M+1): m/z=715.3.

### Example 671

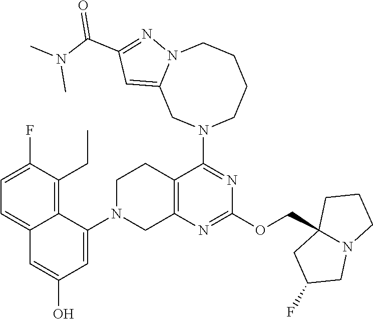 [SMILES: CCc1c(F)ccc2cc(O)cc(N3CCc4c(nc(OC[C@@]56CCCN5C[C@H](F)C6)nc4N4CCCCn5nc(C(=O)N(C)C)cc5C4)C3)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-4,5,6,7,8,9-hexahydropyrazolo[1,5-a][1,4]diazocine-2-carboxamide

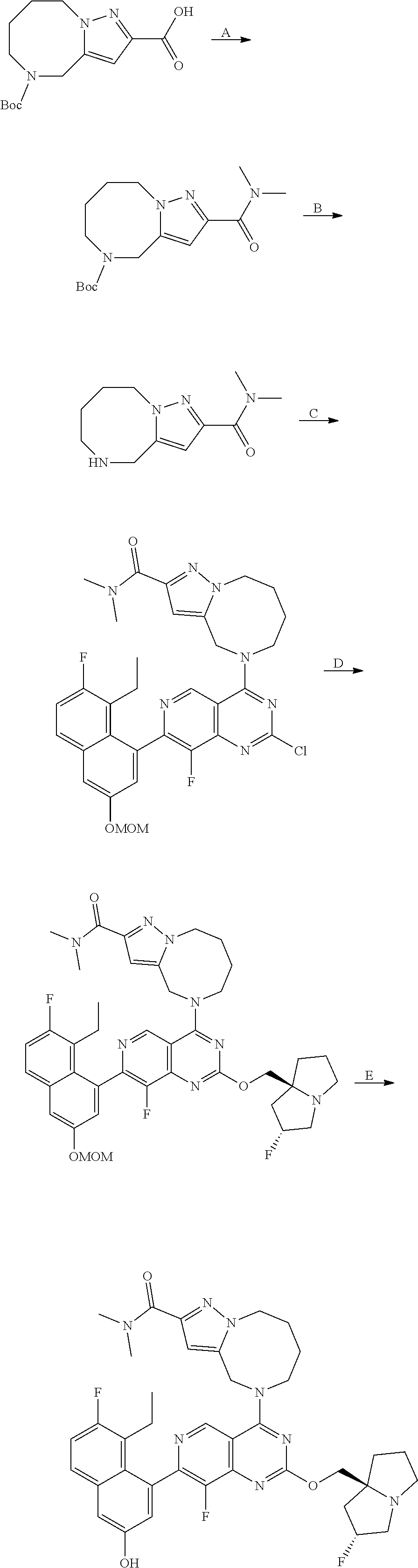

Step A tert-butyl 2-(dimethylcarbamoyl)-6,7,8,9-tetrahydropyrazolo[1,5-a][1,4]diazocine-5(4H)-carboxylate: To a solution of 5-(tert-butoxycarbonyl)-4,5,6,7,8,9-hexahydropyrazolo[1,5-a][1,4]diazocine-2-carboxylic acid (300 mg, 1.0 equiv) in DMF (5.0 mL) were added DIEA (1.3 g, 10 equiv), HATU (1.2 g, 3.0 equiv) and methanamine hydrochloride (172 mg, 1.5 equiv). The reaction was stirred at 25° C. for 1 hour. The mixture was diluted with ethyl acetate (100 mL) and filtered. The filtrate was washed with brine (50 mL×3). The organic phase was dried over anhydrous sodium sulfate, concentrated, and purified by column chromatography (SiO₂, petroleum ether/ethyl acetate=2/1 to 1/0) to afford the title compound (210 mg, 64.1% yield) as a colorless oil; LCMS (ESI, M+1): m/z=323.2.

Step B N,N-dimethyl-4,5,6,7,8,9-hexahydropyrazolo[1,5-a][1,4]diazocine-2-carboxamide: A solution of tert-butyl 2-(dimethylcarbamoyl)-6,7,8,9-tetrahydropyrazolo[1,5-a][1,4]diazocine-5(4H)-carboxylate (190 mg, 1.0 equiv) in MeCN (1.00 mL) and HCl/dioxane (4 M, 1.0 mL) was stirred at 25° C. for 1 hour. The reaction was concentrated to afford the title compound (120 mg, crude), LCMS (ESI, M+1): m/z=223.2.

Step C: 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-4,5,6,7,8,9-hexahydropyrazolo[1,5-a][1,4]diazocine-2-carboxamide: To a solution of 2,4-dichloro-7-[8-ethyl-7-fluoro-3-(methoxymethoxy)-1-naphthyl]-8-fluoro-pyrido[4,3-d]pyrimidine (203 mg, 1.00 equiv) in DCM (2.00 mL) were added N,N-dimethyl-4,5,6,7,8,9-hexahydropyrazolo[1,5-a][1,4]diazocine-2-carboxamide (100 mg, 1.00 equiv) and DIPEA (174 mg, 3.00 equiv). The mixture was stirred at −40° C. for 1 hour. The reaction mixture was partitioned between ethyl acetate (20.0 mL) and water (10.0 mL). The organic phase was separated, concentrated under reduced pressure to give a residue and to afford the title compound (120 mg, 42% yield, 60% purity), LCMS (ESI, M+1): m/z=636.1

Step D: 5-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-4,5,6,7,8,9-hexahydropyrazolo[1,5-a][1.4]diazocine-2-carboxamide: To a solution of 5-(2-chloro-7-(8-ethyl-7-fluoro-3-(methoxymethoxy)naphthalen-1-yl)-8-fluoropyrido[4,3-d]pyrimidin-4-yl)-N,N-dimethyl-4,5,6,7,8,9-hexahydropyrazolo[1,5-a][1,4]diazocine-2-carboxamide (90.0 mg, 1.00 equiv) in THF (3.00 mL) was added t-BuONa (40.8 mg, 3.00 equiv), 4 Å molecular sieves (20.0 mg, 1.0 equiv) and ((2R,7aS)-2-fluorotetrahydro-1H-pyrrolizin-7a(5H)-yl)methanol (22.5 mg, 1.00 equiv). The mixture was stirred at 0° C. for 1 hour. The reaction mixture was partitioned between ethyl acetate (50.0 mL) and water (30.0 mL). The organic phase was separated, concentrated under reduced pressure to give a residue. The residue was purified by prep-TLC (SiO2, DCM/MeOH=10/1) to afford the title compound (60.0 mg, 56% yield), LCMS (ESI, M+1): m/z=759.4

Step E: 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-4,5,6,7,8,9-hexahydropyrazolo[1,5-a][1,4]diazocine-2-carboxamide: To 5-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-4,5,6,7,8,9-hexahydropyrazolo[1,5-a][1,4]diazocine-2-carboxamide (50.0 mg, 1.00 equiv) was added HCl/MeOH (4 M, 3.00 mL). The mixture was stirred at 0° C. for 1 hour. The reaction mixture was concentrated and purified with prep-HPLC (column: Phenomenex C18×75×3 um; mobile phase: [water (FA)-ACN]; B %: 12%-42%, 7 min) to afford the title compound (2.51 mg, 4.80% yield, 96.6% purity) as a white solid; ¹H NMR (400 MHZ, METHANOL-d4) δ=9.06 (s, 1H), 7.67 (dd, J=6.0, 8.8 Hz, 1H), 7.33-7.22 (m, 2H), 7.04 (d, J=2.4 Hz, 1H), 6.71 (s, 1H), 5.42-5.23 (m, 3H), 4.48 (br d, J=2.8 Hz, 2H), 4.34-4.21 (m, 2H), 4.15 (br s, 2H), 3.35 (s, 3H), 3.22 (br d, J=18.6 Hz, 2H), 3.09 (s, 3H), 3.06-2.98 (m, 1H), 2.54-2.42 (m, 1H), 2.38-2.10 (m, 4H), 2.09-1.98 (m, 8H), 1.94-1.83 (m, 1H), 1.35-1.27 (m, 1H), 0.79 (br t, J=7.4 Hz, 3H), LCMS (ESI, M+1): m/z=715.2.

### Example 672

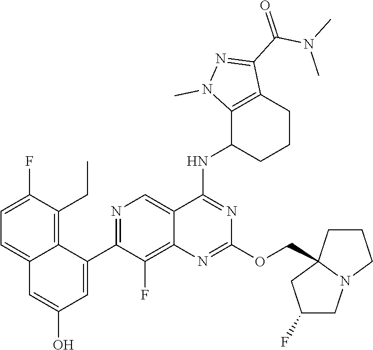 [SMILES: CCc1c(F)ccc2cc(O)cc(-c3ncc4c(NC5CCCc6c(C(=O)N(C)C)nn(C)c65)nc(OC[C@@]56CCCN5C[C@H](F)C6)nc4c3F)c12]

### 7-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)-N,N,1-trimethyl-4,5,6,7-tetrahydro-1H-indazole-3-carboxamide

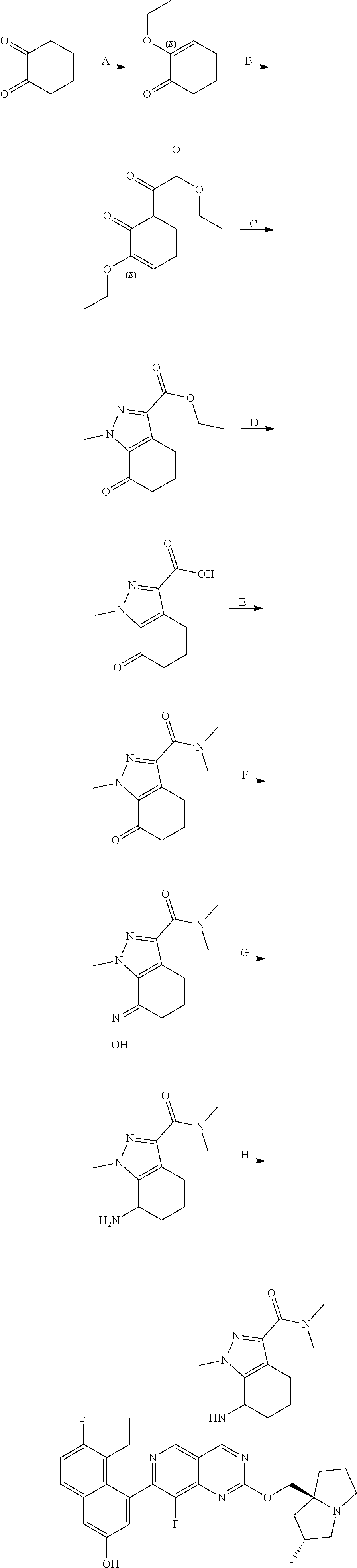

Step A. 2-ethoxycyclohex-2-enone: To a mixture of cyclohexane-1,2-dione (15.0 g, 1.0 equiv) and EtOH (122 g, 19.9 equiv) in toluene (300 mL) was added TsOH (2.80 g, 0.12 equiv) at 25° C. under nitrogen atmosphere. The reaction was stirred at 90° C. for 12 hours. The reaction mixture was diluted with water (20.0 mL) and extracted with ethyl acetate (3×30.0 mL). The combined organic layers were washed with brine (30.0 mL), dried over anhydrous sodium sulfate, filtered, concentrated and purified by silica gel chromatography (petroleum ether/ethyl acetate 1:0 to 20:1) to afford the title compound (9.0 g, 42% yield) as a yellow liquid; ¹H NMR (400 MHZ, dimethylsulfoxide-d₆) δ=5.98 (t, J=4.8 Hz, 1H), 3.71-3.63 (m, 2H), 2.41-2.31 (m, 4H), 1.89-1.81 (m, 2H), 1.22 (t, J=7.2 Hz, 3H); LCMS (ESI, M+1): m/z=141.2.

Step B. ethyl 2-(3-ethoxy-2-oxocyclohex-3-en-1-yl)-2-oxoacetate: To a solution of 2-ethoxycyclohex-2-en-1-one (2.00 g, 1.0 equiv) in THF (20.0 mL) was added LiHMDS (3.1 g, 1.3 equiv) at −60° C. After stirring at −60° C. for 30 minutes, a solution of diethyl oxalate (2.7 g, 1.3 equiv) in THF (5.0 mL) was added to the solution. The reaction was stirred at 20° C. for 1 hour. The reaction mixture was poured into ice-water (50 mL), extracted with ethyl acetate (3×30 mL). The combined organic layers were washed with brine (100.0 mL), dried over anhydrous sodium sulfate, filtered, concentrated and purified by silica gel chromatography (petroleum ether/ethyl acetate 1:0 to 20:1) to afford the title compound (2.30 g, 67% yield) as a yellow oil; LCMS (ESI, M+1): m/z=241.1

Step C. ethyl 1-methyl-7-oxo-4,5,6,7-tetrahydro-1H-indazole-3-carboxylate: To a mixture of ethyl 2-(3-ethoxy-2-oxo-cyclohex-3-en-1-yl)-2-oxo-acetate (1.80 g, 1.0 equiv) in AcOH (10.0 mL) was added methylhydrazine (949 mg, 1.1 equiv) under nitrogen. The reaction was stirred at 25° C. for 1 hour. The solvent was concentrated. The residue was dissolved in water (30 mL), adjusted to pH=8 with 30% NH₄OH and extracted with DCM (3×40 mL). The organic layer was dried over anhydrous sodium sulfate, concentrated and purified by silica gel chromatography (petroleum ether/ethyl acetate 10:1 to 4:1) to afford the title compound (1.50 g, 90% yield) as a white solid; ¹H NMR (400 MHZ, dimethylsulfoxide-d₆) δ=4.35-4.21 (m, 2H), 4.10 (s, 3H), 2.92 (t, J=6.0 Hz, 2H), 2.53 (br d, J=4.8 Hz, 2H), 2.11-1.99 (m, 2H), 1.30 (t, J=7.2 Hz, 3H); LCMS (ESI, M+1): m/z=222.9.

Step D. 1-methyl-7-oxo-4,5,6,7-tetrahydro-1H-indazole-3-carboxylic acid: To a mixture of ethyl 1-methyl-7-oxo-5,6-dihydro-4H-indazole-3-carboxylate (1.0 g, 1.0 equiv) in THF (18.0 mL) was added NaOH (2.0 M, 9.0 mL, 4.0 equiv) at 20° C. under nitrogen. The reaction was stirred at 20° C. for 1 hour. The mixture was diluted with water (2 mL) and adjusted to pH=5 with 1N HCl aqueous solution and extracted with ethyl acetate (3×3.0 mL). The combined organic layers were filtered and concentrated to afford the title compound (0.9 g, 97% yield) as a yellow solid.

Step E. N,N,1-trimethyl-7-oxo-4,5,6,7-tetrahydro-1H-indazole-3-carboxamide: To a mixture of 1-methyl-7-oxo-5,6-dihydro-4H-indazole-3-carboxylic acid (0.90 g, 1.0 equiv) in DMF (8.0 mL) were added DIPEA (2.80 g, 5.0 equiv) and HATU (3.30 g, 2.0 equiv) at 20° C. under nitrogen. After stirring at 20° C. for 5 minutes, N-methylmethanamine (2.0 M, 4.4 mL, 2.0 equiv) was added. The reaction was stirred at 20° C. for 2 hours. The reaction mixture was diluted with water (20 mL) and extracted with ethyl acetate (3×30.0 mL). The combined organic layers were washed with brine (30.0 mL), dried over anhydrous sodium sulfate, filtered, concentrated and purification by silica gel chromatography (petroleum ether/ethyl acetate 10:1 to 1:1) to afford the title compound (1.20 g, 99% yield) as yellow oil; ¹H NMR (400 MHZ, dimethylsulfoxide-d₆) δ=4.06 (s, 3H), 3.18 (s, 3H), 2.97 (s, 3H), 2.78 (t, J=6.0 Hz, 2H), 2.54-2.51 (m, 2H), 2.05-1.97 (m, 2H); LCMS (ESI, M+1): m/z=222.2.

Step F. (E)-7-(hydroxyimino)-N,N,1-trimethyl-4.5,6,7-tetrahydro-1H-indazole-3-carboxamide: To a mixture of N,N, 1-trimethyl-7-oxo-5,6-dihydro-4H-indazole-3-carboxamide (0.6 g, 1.0 equiv) and hydroxylamine hydrochloride (376.9 mg. 2.0 equiv) in EtOH (8.0 mL) was added Pyridine (643.5 mg, 3.0 equiv) at 20° C. under nitrogen. The reaction was stirred at 80° C. for 1 hour. The mixture was diluted with water (20.0 mL) and extracted with ethyl acetate (3×30.0 mL). The combined organic layers were washed with brine (30.0 mL), dried over anhydrous sodium sulfate, filtered, concentrated and purified by reversed phase chromatography [water (0.1% formic acid)/acetonitrile)] to afford the title compound (385 mg, 58% yield) as a white solid; ¹H NMR (400 MHZ, dimethylsulfoxide-d₆) δ=11.33 (s, 1H), 4.00 (s, 3H), 3.18 (s, 3H), 2.95 (s, 3H), 2.68-2.55 (m, 4H), 1.78-1.69 (m, 2H), LCMS (ESI, M+1): m/z=236.8.

Step G. 7-amino-N,N,1-trimethyl-4,5,6,7-tetrahydro-1H-indazole-3-carboxamide: To a solution of (7E)-7-hydroxyimino-N,N,1-trimethyl-5,6-dihydro-4H-indazole-3-carboxamide (300 mg, 1.0 equiv) in MeOH (2.0 mL) and THF (1.0 mL) was added Pd(OH)₂/C (100 mg, 20% purity, 0.56 equiv) under nitrogen. The reaction was stirred at 60° C. for 60 hours under hydrogen (45 psi). The mixture was filtered, concentrated and purified by reversed phase chromatography [water (0.1% formic acid)/acetonitrile)] to afford the title compound (65.0 mg, 23% yield) as a white solid; LCMS (ESI, M+1): m/z=223.1

Step H. 7-((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)-N,N,1-trimethyl-4,5,6,7-tetrahydro-1H-indazole-3-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) in DMF (0.1 mL) were added DIPEA (218 mg, 20.0 equiv) and 7-amino-N,N,1-trimethyl-4,5,6,7-tetrahydroindazole-3-carboxamide (37.5 mg, 2.0 equiv) under nitrogen. The reaction was stirred at 40° C. for 2 hours. The mixture was purified by prep-HPLC [Phenomenex luna C18 150×25 mm×10 μm; A: water (FA), B: ACN; B %: 20%-50% over 10 min] and lyophilized to afford the title compound (15.0 mg, 22% yield, 0.46FA) as a white solid; SFC: Chiralcel OJ-3 50×4.6 mm I.D., 3 μm, Gradient elution: MeOH (0.05% DEA) in CO₂ from 5% to 40%, 3 mL/min, 220 nm, t_{R}: 1.548 min, 1.617 min; ¹H NMR (400 MHZ, dimethylsulfoxide-d₆) δ=9.41 (d, J=2.4 Hz, 1H), 9.34-9.21 (m, 1H), 7.84-7.70 (m, 1H), 7.42-7.28 (m, 2H), 6.98 (d, J=2.4 Hz, 1H), 5.83-5.68 (m, 1H), 5.42-5.16 (m, 1H), 4.25-4.06 (m, 2H), 3.71 (d, J=16.4 Hz, 3H), 3.25 (br s, 3H), 3.13-3.00 (m, 4H), 2.97 (s, 3H), 2.87-2.64 (m, 3H), 2.41-2.35 (m, 1H), 2.23-2.10 (m, 2H), 2.06-1.92 (m, 3H), 1.90-1.74 (m, 5H), 0.78-0.67 (m, 3H); ¹⁹F NMR (400 MHZ, dimethylsulfoxide-d₆)=−119.679, −139.505, −172.081; LCMS (ESI, M+1): m/z=715.1.

### Example 673

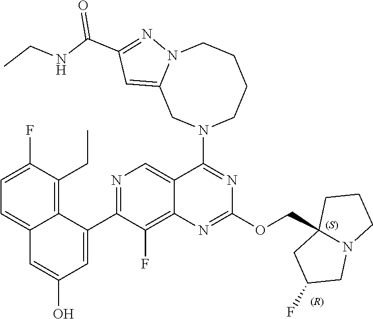

### N-ethyl-5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,5,6,7,8,9-hexahydropyrazolo[1,5-a][1,4]diazocine-2-carboxamide

Synthesized according to Example 671. The title compound was obtained as a white solid; ¹H NMR (400 MHZ, METHANOL-d₄) δ=9.08 (s, 1H), 7.68 (dd, J=6.0, 9.1 Hz, 1H), 7.30 (d, J=2.8 Hz, 1H), 7.25 (t, J=9.2 Hz, 1H), 7.05 (d, J=2.4 Hz, 1H), 6.80 (s, 1H), 5.42-5.23 (m, 3H), 4.45 (br d, J=4.4 Hz, 2H), 4.31-4.11 (m, 4H), 3.39 (q, J=7.2 Hz, 2H), 3.28-3.17 (m, 2H), 3.10-2.98 (m, 1H), 2.56-1.76 (m, 13H), 1.21 (t, J=7.2 Hz, 3H), 0.79 (t, J=7.6 Hz, 3H); LCMS (ESI, M+1): m/z=715.5.

### Example 674

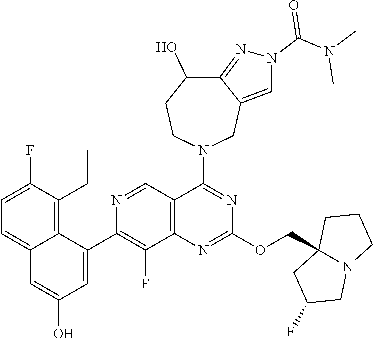 [SMILES: CCc1c(F)ccc2cc(O)cc(-c3ncc4c(N5CCC(O)c6nn(C(=O)N(C)C)cc6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-8-hydroxy-N,N-dimethyl-5,6,7,8-tetrahydropyrazolo[4,3-c]azepine-2(4H)-carboxamide

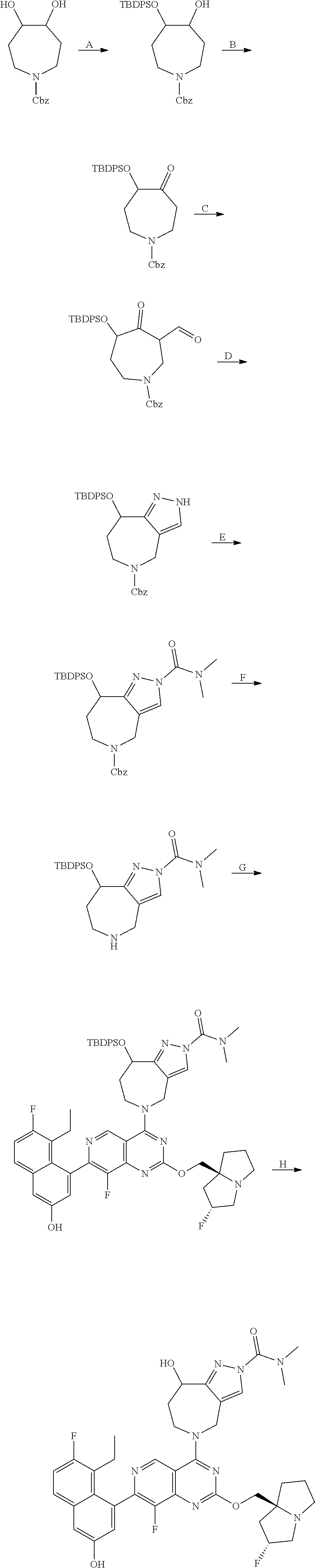

Step A. benzyl 4-((tert-butyldiphenylsilyl)oxy)-5-hydroxyazepane-1-carboxylate: To a solution of benzyl 4,5-dihydroxyazepane-1-carboxylate (1.50 g, 1.0 equiv), imidazole (1.15 g, 3.0 equiv) and DMAP (6.91 mg, 0.01 equiv) in DCM (1 mL) was added TBDPS-Cl (1.09 g, 0.7 equiv) at 0° C. The mixture was stirred at 25° C. for 2 hours. The mixture was diluted with water (20 mL) and extracted with ethyl acetate (4×20 mL). The combined organic layers were dried over anhydrous sodium sulfate, concentrated and purified by column chromatography [SiO₂, Petroleum ether/Ethyl acetate 3:1 to 1:1] to afford the title compound (1.20 g, 41% yield) as a yellow liquid; ¹H NMR (400 MHZ, CHLOROFORM-d) δ=7.66-7.17 (m, 15H), 5.12-4.93 (m, 2H), 3.82-3.79 (m, 2H), 3.67-3.43 (m, 1H), 3.38-3.06 (m, 3H), 2.29-2.19 (m, 1H), 2.11-2.01 (m, 1H), 1.97-1.87 (m, 1H), 1.62-1.39 (m, 2H), 1.03 (br d, J=2.0 Hz, 9H); LCMS (ESI, M+1): m/z=504.3.

Step B. benzyl 4-((tert-butyldiphenylsilyl)oxy)-5-oxoazepane-1-carboxylate: To a solution of benzyl 4-((tert-butyldiphenylsilyl)oxy)-5-hydroxyazepane-1-carboxylate (1.20 g, 1.0 equiv) in DCM (12 mL) was added Dess-Martin reagent (3.03 g, 3.0 equiv). The mixture was stirred at 25° C. for 2 hours. The reaction mixture was filtered. The filtrate was diluted with water (15 mL) and extracted with ethyl acetate (4×15 mL). The combined organic layers were dried over anhydrous sodium sulfate, concentrated and purified by column chromatography [SiO₂, Petroleum ether/Ethyl acetate 5:1] to afford the title compound (900 mg, 75% yield) as a white liquid; ¹H NMR (400 MHZ, CHLOROFORM-d) δ=7.57-7.44 (m, 15H), 5.05-4.94 (m, 2H), 4.41-4.27 (m, 1H), 3.95-3.57 (m, 3H), 3.46-3.25 (m, 1H), 2.80-2.40 (m, 2H), 1.71-1.41 (m, 2H), 1.05 (s, 9H).

Step C. benzyl 5-((tert-butyldiphenylsilyl)oxy)-3-formyl-4-oxoazepane-1-carboxylate: A solution of 1-tert-butoxy-N,N,N′,N′-tetramethylmethanediamine and benzyl 4-((tert-butyldiphenylsilyl)oxy)-5-oxoazepane-1-carboxylate (2.0 g, 1.0 equiv) in THF (25 mL) was stirred at 60° C. for 8 hours. The mixture was diluted with water (10 mL) and extracted with ethyl acetate (4×10 mL). The organic phase was dried over anhydrous sodium sulfate, concentrated to afford the title compound (2.0 g, crude) as a yellow oil.

Step D. benzyl 8-((tert-butyldiphenylsilyl)oxy)-4,6,7,8-tetrahydropyrazolo[4,3-c]azepine-5 (2H)-carboxylate: To a solution of benzyl 5-((tert-butyldiphenylsilyl)oxy)-3-formyl-4-oxoazepane-1-carboxylate (200 mg, 1.0 equiv) in EtOH (3 mL) was added N₂H₄·H₂O (630 mg, 85% purity, 28 equiv). The mixture was stirred at 75° C. for 12 hours. The mixture was diluted with water (5 mL) and extracted with ethyl acetate (4×5 mL). The organic phase was dried over anhydrous sodium sulfate, concentrated and purified by reversed phase flash chromatography [C18, 0.1% formic acid condition] to afford the title compound (20.0 mg, 10% yield) as a white oil; ¹H NMR (400 MHZ, CHLOROFORM-d) δ=7.60-7.20 (m, 16H), 5.08-4.88 (m, 3H), 4.78-4.48 (m, 1H), 4.29-4.08 (m, 1H), 3.86 (br d, J=6.4 Hz, 2H), 1.88-1.61 (m, 2H), 0.97-0.96 (m, 9H).

Step E. benzyl 8-((tert-butyldiphenylsilyl)oxy)-2-(dimethylcarbamoyl)-4,6,7,8-tetrahydropyrazolo[4,3-c]azepine-5 (2H)-carboxylate: To a solution of benzyl 8-((tert-butyldiphenylsilyl)oxy)-4,6,7,8-tetrahydropyrazolo[4,3-c]azepine-5 (2H)-carboxylate (10.0 mg, 1.0 equiv) in THF (0.5 mL) was added NaH (2.28 mg, 60% purity, 3.0 equiv) at 0° C. The mixture was stirred at 0° C. for 0.5 hour. Then dimethylcarbamic chloride (4.09 mg, 2.0 equiv) was added into the mixture. The mixture was stirred at 0° C. for 0.5 hour. The mixture was diluted with water (5 mL) and extracted with ethyl acetate (4×5 mL). The organic phase was dried over anhydrous sodium sulfate, concentrated and purified by reversed phase flash chromatography [C18, 0.1% formic acid condition] to afford the title compound (10.0 mg, crude) as a yellow oil; ¹H NMR (400 MHz, CHLOROFORM-d) δ=7.96-7.75 (m, 1H), 7.61-7.60 (m, 2H), 7.40-7.11 (m, 13H), 5.09-4.75 (m, 4H), 4.19-4.07 (m, 1H), 4.03-3.97 (m, 1H), 3.87-3.69 (m, 1H), 2.91 (s, 6H), 1.89-1.79 (m, 1H), 1.77-1.62 (m, 1H), 0.96 (br s, 9H); LCMS (ESI, M+1): m/z=597.4.

Step F. 8-((tert-butyldiphenylsilyl)oxy)-N,N-dimethyl-5,6,7,8-tetrahydropyrazolo[4,3-c]azepine-2(4H)-carboxamide: To a solution of Pd/C (50.0 mg, 10% purity) in MeOH (2 mL) was added benzyl 8-((tert-butyldiphenylsilyl)oxy)-2-(dimethylcarbamoyl)-4,6,7,8-tetrahydropyrazolo[4,3-c]azepine-5 (2H)-carboxylate (200 mg, 1.0 equiv) under N₂ atmosphere. The suspension was degassed and purged with H₂ for 3 times. The mixture was stirred under H₂ (15 Psi) at 25° C. for 2 hours. The reaction mixture was filtered and concentrated to afford the title compound (300 mg, crude) as a yellow oil.

Step G. 8-((tert-butyldiphenylsilyl)oxy)-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydropyrazolo[4,3-c]azepine-2(4H)-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (130 mg, 1.0 equiv) and 8-((tert-butyldiphenylsilyl)oxy)-N,N-dimethyl-5,6,7,8-tetrahydropyrazolo[4,3-c]azepine-2(4H)-carboxamide (152 mg, 1.5 equiv) in DMF (1.5 mL) were added DIPEA (85.0 mg, 3.0 equiv) and 4 Å MS (30 mg). The mixture was stirred at 40° C. for 24 hours. The reaction mixture was filtered and purified by reversed phase flash chromatography [C18, 0.1% formic acid condition] to afford the title compound (50.0 mg, 21% yield) as a yellow solid; LCMS (ESI, M+1): m/z=955.4.

Step H. 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-8-hydroxy-N,N-dimethyl-5,6,7,8-tetrahydropyrazolo[4,3-c]azepine-2(4H)-carboxamide: To a solution of 8-((tert-butyldiphenylsilyl)oxy)-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydropyrazolo[4,3-c]azepine-2(4H)-carboxamide (50.0 mg, 1.0 equiv) in DMF (0.5 mL) was added CsF (80.0 mg, 10 equiv). The mixture was stirred at 40° C. for 12 hours. The reaction mixture was filtered and purified by prep-HPLC [Waters Xbridge 150×25 mm×5 μm; A: water (10 mM NH₄HCO₃), B: ACN, B %: 33%-60% over 9 min] and lyophilized to afford the title compound (7.51 mg, 20% yield) as a white solid; ¹H NMR (400 MHZ, METHANOL-d₄) δ−9.18 (d, J=2.4 Hz, 1H), 8.25 (d, J=6.0 Hz, 1H), 7.69-7.65 (m, 1H), 7.34-7.18 (m, 2H), 7.07-7.04 (m, 1H), 5.40-5.22 (m, 2H), 5.11-5.06 (m, 1H), 4.98-4.90 (m, 2H), 4.54-4.44 (m, 1H), 4.34-4.19 (m, 2H), 3.39-3.33 (m, 1H), 3.25 (br s, 1H), 3.20 (br s, 6H), 3.28-3.12 (m, 1H), 3.06-2.98 (m, 1H), 2.52-2.34 (m, 3H), 2.32-2.24 (m, 1H), 2.23-2.12 (m, 3H), 2.04-1.95 (m, 2H), 1.94-1.83 (m, 1H), 0.78 (br t, J=7.1 Hz, 3H); LCMS (ESI, M+1): m/z=717.4.

### Example 675

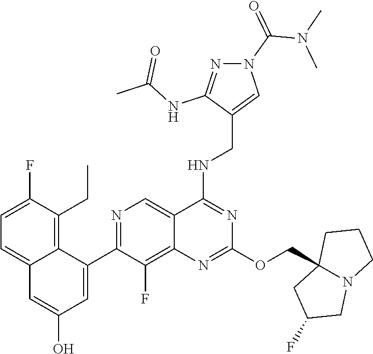 [SMILES: CCc1c(F)ccc2cc(O)cc(-c3ncc4c(NCc5cn(C(=O)N(C)C)nc5NC(C)=O)nc(OC[C@@]56CCCN5C[C@H](F)C6)nc4c3F)c12]

### 3-acetamido-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide

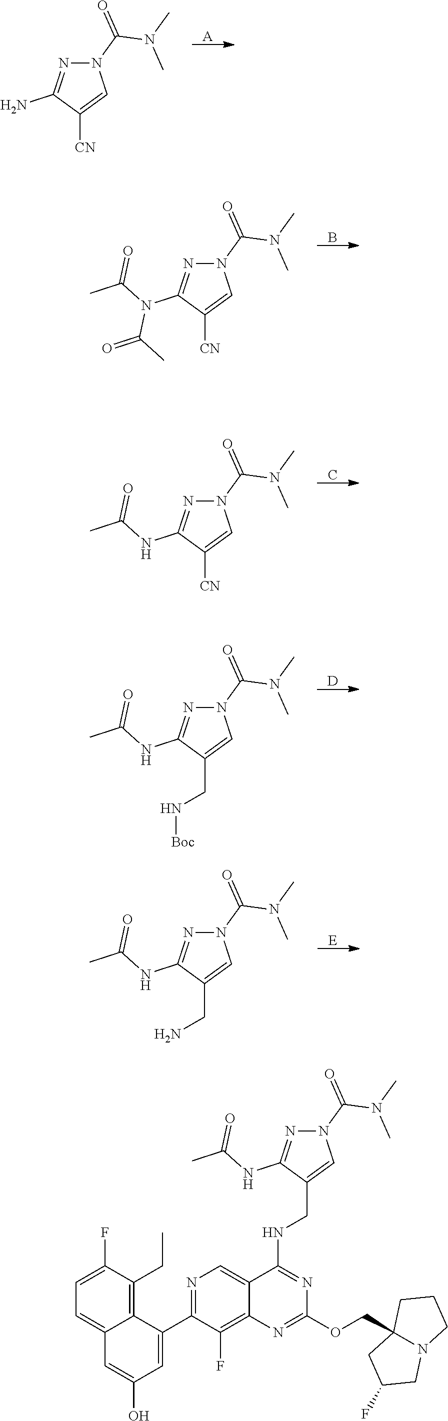

Step A. 3-(N-acetylacetamido)-4-cyano-N,N-dimethyl-1H-pyrazole-1-carboxamide: To a mixture of 3-amino-4-cyano-N,N-dimethyl-1H-pyrazole-1-carboxamide, Intermediate 15 (500 mg, 1.0 equiv) in Ac₂O (5.00 mL) was added TEA (847 mg, 3.0 equiv) dropwise at 0° C. The reaction was stirred at 60° C. for 4 hours. The mixture was quenched with water (10.0 ml) and extracted with EtOAc (3×10.0 mL). The combined organic layers were dried over anhydrous sodium sulfate and concentrated to afford the title compound (644 mg, crude) as a yellow solid; ¹H NMR (400 MHZ, dimethylsulfoxide-d₆) δ=9.18 (s, 1H), 3.07 (s, 6H), 2.31 (s, 6H).

Step B. 3-acetamido-4-cyano-N,N-dimethyl-1H-pyrazole-1-carboxamide: To a mixture of 3-(N-acetylacetamido)-4-cyano-N,N-dimethyl-1H-pyrazole-1-carboxamide (644 mg, 1.0 equiv) in MeOH (6.50 mL) was added NH₃·H₂O (1.30 mL). The reaction was degassed and stirred at 25° C. for 2 hours. The mixture was concentrated to afford the title compound (570 mg, 99% yield) as a yellow solid.

Step C. tert-butyl ((3-acetamido-1-(dimethylcarbamoyl)-1H-pyrazol-4-yl)methyl)carbamate: To a mixture of 3-acetamido-4-cyano-N,N-dimethyl-1H-pyrazole-1-carboxamide (570 mg, 1.0 equiv) in MeOH (8.00 mL) were added (Boc)₂O (1.69 g, 3.0 equiv) and Pd/C (200 mg, 10% purity) under nitrogen atmosphere. The reaction was degassed and purged with hydrogen 3 times. The reaction was stirred under hydrogen (15 psi) at 25° C. for 24 hours. The mixture was filtered and purified by silica gel chromatography (petroleum ether/ethyl acetate 10:1 to dichloromethane/methanol 10:1) followed by reversed phase flash chromatography [C18, 0.1% formic acid condition] to afford the title compound (160 mg, 17% yield) as a yellow solid; ¹H NMR (400 MHZ, dimethylsulfoxide-d₆) δ=10.05 (br s, 1H), 7.88 (s, 1H), 7.12 (br s, 1H), 3.88 (br d, J=5.6 Hz, 2H), 3.10 (br s, 6H), 2.03 (s, 3H), 1.38 (s, 9H).

Step D. 3-acetamido-4-(aminomethyl)-N,N-dimethyl-1H-pyrazole-1-carboxamide: To a mixture of tert-butyl ((3-acetamido-1-(dimethylcarbamoyl)-1H-pyrazol-4-yl)methyl)carbamate (140 mg, 1.0 equiv) in DCM (1.50 mL) was added TFA (2.45 g, 50 equiv) dropwise at 0° C. The reaction was stirred at 25° C. for 0.5 hour. The mixture was filtered and concentrated to afford the title compound (169 mg, TFA) as a yellow oil; ¹H NMR (400 MHZ, dimethylsulfoxide-d₆) δ=10.51 (br s, 1H), 8.23 (s, 1H), 7.93 (br s, 2H), 3.83 (br d, J=5.6z, 2H), 3.11 (br s, 6H), 2.08 (s, 3H).

Step E. 3-acetamido-4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-1-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (100 mg, 1.0 equiv) and 3-acetamido-4-(aminomethyl)-N,N-dimethyl-1H-pyrazole-1-carboxamide (114 mg, 3.0 equiv) in DMF (0.50 mL) and ACN (0.50 mL) were added K₃PO₄ (107 mg, 3.0 equiv) and 4 Å molecular sieves (50.0 mg, 1.0 equiv). The reaction was stirred at 40° C. for 24 hours. The mixture was filtered and purified with prep-HPLC [Phenomenex Luna C18 150×25 mm×5 μm; A: water (FA), B: ACN; B %: 28%-58% over 10 min] to afford the title compound (24.8 mg, 20% yield) as a white solid; SFC: >99% ee, Chiralcel OJ-3 50×4.6 mm I.D., 3 μm; Gradient elution: MeOH (0.05% DEA) in CO₂ from 5% to 40%, 3 mL/min, 220 nm, t_{R}: 1.535 min; ¹H NMR (400 MHZ, dimethylsulfoxide-d₆) δ=10.79-9.37 (m, 1H), 9.33-9.12 (m, 1H), 8.15 (s, 1H), 7.68 (dd, J=6.0, 9.2 Hz, 1H), 7.29 (t, J=9.6 Hz, 1H), 7.23 (d, J=2.4 Hz, 1H), 6.96 (d, J=2.4 Hz, 1H), 5.41-5.11 (m, 1H), 4.58 (br d, J=8.4 Hz, 2H), 4.17-4.11 (m, 1H), 4.09-4.02 (m, 1H), 3.19-2.98 (m, 9H), 2.87-2.75 (m, 1H), 2.40-2.28 (m, 1H), 2.21-1.94 (m, 7H), 1.88-1.69 (m, 3H), 0.70 (t, J=7.2 Hz, 3H); ¹⁹F NMR (400 MHZ, dimethylsulfoxide-d₆) δ=−139.519, −171.851; LCMS (ESI, M+1): m/z=718.3.

### Example 676

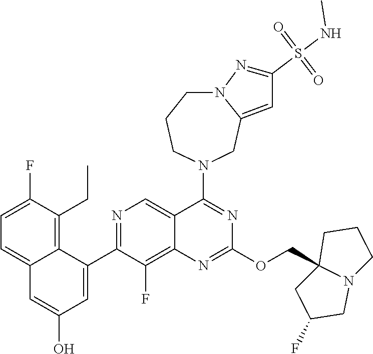 [SMILES: CCc1c(F)ccc2cc(O)cc(-c3ncc4c(N5CCCn6nc(S(=O)(=O)NC)cc6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N-methyl-5,6,7,8-tetrahydro-4H-pyrazolo[1,5-a][1,4]diazepine-2-sulfonamide

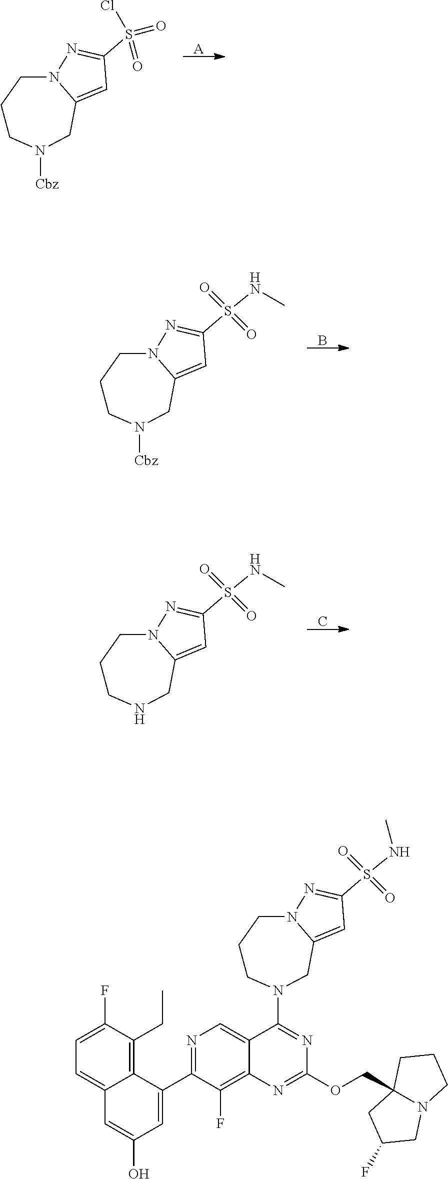

Step A. benzyl 2-(N-methylsulfamoyl)-7,8-dihydro-4H-pyrazolo[1,5-a][1,4]diazepine-5(6H)-carboxylate: To a mixture of methylamine hydrochloride (109 mg, 3.0 equiv) and K₂CO₃ (448 mg, 6.0 equiv) in THF (1.2 mL) and H₂O (3.6 mL) was added a solution of benzyl 2-(chlorosulfonyl)-7,8-dihydro-4H-pyrazolo[1,5-a][1,4]diazepine-5(6H)-carboxylate (200 mg, 1.0 equiv) in THF (2.3 mL) dropwise below 5° C. The reaction was stirred at 5-15° C. for 14 hours. The mixture was diluted with H₂O (10 mL) and extracted with EtOAc (3×10 mL). The combined organic layers were washed with 1N HCl (10 mL) and brine (10 mL), dried over anhydrous sodium sulfate, concentrated, and purified by reversed phase flash chromatography [C18, 0.1% formic acid condition] to afford the title compound (70 mg, 35% yield) as a yellow oil; ¹H NMR (400 MHZ, chloroform-d) δ=7.42-7.28 (m, 5H), 6.71-6.49 (m, 1H), 5.15-5.08 (m, 2H), 4.60-4.52 (m, 2H), 4.52-4.41 (m, 3H), 3.87-3.77 (m, 2H), 2.81-2.73 (m, 3H), 2.04-1.93 (m, 2H); LCMS (ESI, M−1): m/z=364.9.

Step B. N-methyl-5,6,7,8-tetrahydro-4H-pyrazolo[1,5-a][1,4]diazepine-2-sulfonamide: To a mixture of benzyl 2-(N-methylsulfamoyl)-7,8-dihydro-4H-pyrazolo[1,5-a][1,4]diazepine-5(6H)-carboxylate (80 mg, 1.0 equiv) in MeOH (5 mL) were added NH₃·MeOH (12 M, 0.5 mL) and Pd/C (30 mg, 10% purity) under nitrogen. The reaction was degassed and purged with hydrogen 3 times. The reaction was stirred at 20° C. for 1 hours under hydrogen (15 psi) atmosphere. The mixture was filtered and concentrated to afford the title compound (43 mg, crude) as a yellow oil.

Step C. 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N-methyl-5,6,7,8-tetrahydro-4H-pyrazolo[1,5-a][1,4]diazepine-2-sulfonamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (45 mg, 1.0 equiv) and N-methyl-5,6,7,8-tetrahydro-4H-pyrazolo[1,5-a][1,4]diazepine-2-sulfonamide (35 mg, 2.0 equiv) in DMF (0.2 mL) and ACN (0.2 mL) were added 4 Å molecular sieves (20 mg) and K₃PO₄ (48 mg, 3.0 equiv). The reaction was stirred at 40° C. for 24 hours. The mixture was filtered and purified with HPLC [Phenomenex luna C18 150×25 mm×10 μm; A: water (NH₄HCO₃), B: ACN; B %: 36%-66% over 8 min] to afford the title compound (16.1 mg, 29% yield) as an off-white solid; SFC: >99% ee, Chiralcel OJ-3 50×4.6 mm I.D., 3 μm; Gradient elution: MeOH (0.05% DEA) in CO₂ from 5% to 40%; 3 mL/min; 220 nm; t_{R}: 1.947 min; ¹H NMR (400 MHZ, methanol-d₄) δ=9.19-9.16 (m, 1H), 7.71-7.65 (m, 1H), 7.32-7.29 (m, 1H), 7.28-7.21 (m, 1H), 7.06-7.03 (m, 1H), 6.85-6.82 (m, 1H), 5.41-5.16 (m, 3H), 4.63-4.53 (m, 2H), 4.52-4.38 (m, 2H), 4.30-4.25 (m, 1H), 4.25-4.20 (m, 1H), 3.28 (br d, J=1.6 Hz, 2H), 3.16 (s, 1H), 3.06-2.96 (m, 1H), 2.69-2.62 (m, 3H), 2.54-2.40 (m, 3H), 2.35-2.23 (m, 1H), 2.23-2.09 (m, 3H), 2.05-1.87 (m, 3H), 0.83-0.74 (m, 3H); ¹⁹F NMR (400 MHZ, methanol-d₄) δ=−121.145, −138.407, −173.621; LCMS (ESI, M+1): m/z=723.4.

### Example 677

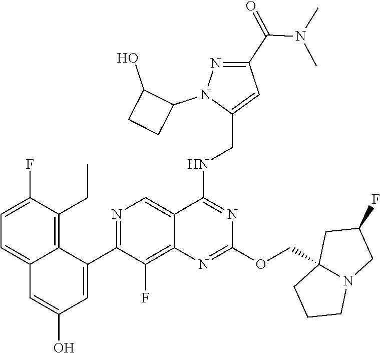 [SMILES: CCc1c(F)ccc2cc(O)cc(-c3ncc4c(NCc5cc(C(=O)N(C)C)nn5C5CCC5O)nc(OC[C@@]56CCCN5C[C@H](F)C6)nc4c3F)c12]

### 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1-(2-hydroxycyclobutyl)-N,N-dimethyl-1H-pyrazole-3-carboxamide

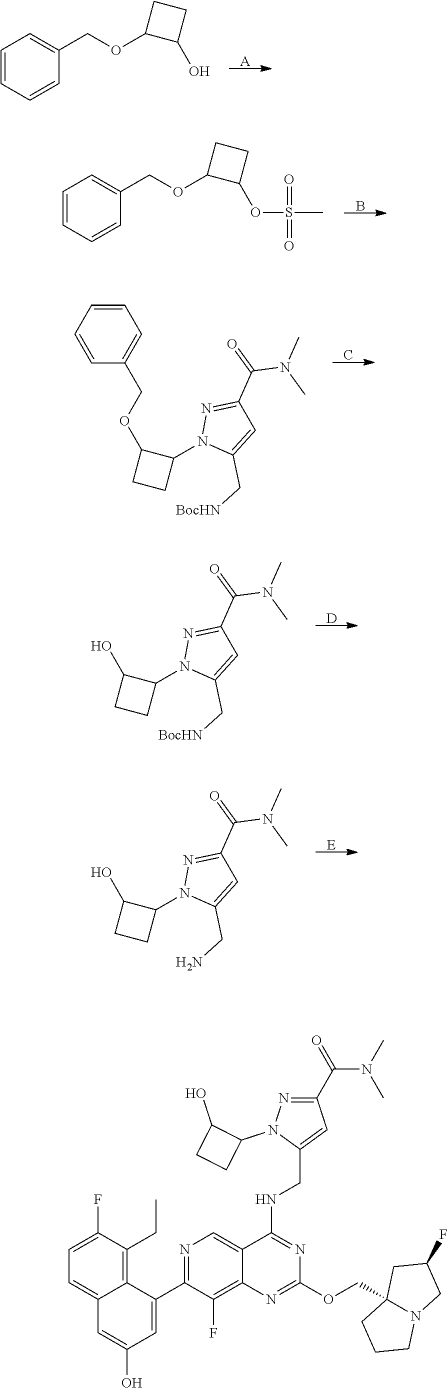

Step A. (2-benzyloxycyclobutyl) methanesulfonate: To a solution of 2-benzyloxycyclobutanol (1 g, 1.0 equiv) and TEA (1.7 g, 2.3 mL, 3.0 equiv) in DCM (10 mL) was added MsCl (964 mg, 1.5 equiv) dropwise at 0° C. under N₂. The reaction mixture was warmed to 25° C. and stirred at 25° C. for 2 hours. The reaction was quenched by iced water slowly and then extracted with DCM (20 mL×3). The combined organic phases were washed with brine (25 mL), dried over anhydrous Na₂SO₄, filtered and concentrated in vacuo to afford the title compound (1.3 g, crude) as a yellow oil. ¹H NMR (400 MHZ, CHLOROFORM-d) δ 2.04-2.15 (m, 1H) 2.18-2.29 (m, 2H) 2.30-2.45 (m, 1H) 3.04 (s, 3H) 4.22-4.28 (m, 1H) 4.59 (q, J=11.6 Hz, 2H) 5.11-5.18 (m, 1H) 7.33-7.40 (m, 5H).

Step B. tert-butyl N-[[2-[(1R,2S)-2-benzyloxycyclobutyl]-5-(dimethylcarbamoyl) pyrazol-3-yl]methyl]carbamate: To a solution of tert-butyl N-[[3-(dimethylcarbamoyl)-1H-pyrazol-5-yl]methyl]carbamate (325 mg, 1.0 equiv) and (2-benzyloxycyclobutyl) methanesulfonate (620.9 mg, 2.0 equiv) in DMF (4 mL) was added 4 Å molecular sieves (250 mg) and Cs₂CO₃ (1.2 g, 3.0 equiv) at 25° C. The mixture was stirred at 80° C. for 12 hours. The residue was purified by prep-HPLC [C18, 0.1% NH₄HCO₃ condition] to afford the title compound (76 mg, 8% yield) as a yellow solid. ¹H NMR (400 MHZ, METHANOL-d₄) δ 1.43 (s, 9H) 1.68-1.80 (m, 1H) 1.91-2.01 (m, 1H) 2.14-2.27 (m, 2H) 3.07 (s, 3H) 3.26-3.30 (m, 3H) 4.18-4.33 (m, 2H) 4.39-4.47 (m, 2H) 4.47-4.56 (m, 1H) 4.70-4.78 (m, 1H) 6.50 (s, 1H) 7.18-7.31 (m, 5H).

Step C. tert-butyl N-[[5-(dimethylcarbamoyl)-2-(2-hydroxycyclobutyl) pyrazol-3-yl]methyl]carbamate: To a solution of tert-butyl N-[[2-(2-benzyloxycyclobutyl)-5-(dimethylcarbamoyl) pyrazol-3-yl]methyl]carbamate (76.0 mg, 1.0 equiv) in MeOH (2 mL) was added Pd/C (100 mg, 10% purity) under N₂ at 25° C. The suspension was degassed under vacuum and purged with H₂ several times. The mixture was stirred under H₂ (15 Psi) at 40° C. for 2 hours. The reaction mixture was filtered, and the filterate was concentrated to afford the title compound (54 mg, crude) as a white solid.

Step D. 5-(aminomethyl)-1-(2-hydroxycyclobutyl)-N,N-dimethyl-pyrazole-3-carboxamide: To a solution of tert-butyl N-[[5-(dimethylcarbamoyl)-2-(2-hydroxycyclobutyl) pyrazol-3-yl]methyl]carbamate (60.0 mg, 1.0 equiv) in dioxane (2 mL) was added HCl/dioxane (4 M, 44.3 μL, 1.0 equiv) at 25° C. The mixture was stirred at 25° C. for 0.5 hour. The reaction mixture was filtered, and the filterate was concentrated to afford the title compound (42 mg, crude, HCl) as a white solid.

Step E. 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1-(2-hydroxycyclobutyl)-N,N-dimethyl-1H-pyrazole-3-carboxamide: To a solution of 5-ethyl-4-[8-fluoro-2-[[(2R,8S)-2-fluoro-1,2,3,5,6,7-hexahydropyrrolizin-8-yl]methoxy]-4-(2,2,2-trifluoroethoxy)pyrido[4,3-d]pyrimidin-7-yl]naphthalen-2-ol (20 mg, 1.0 equiv) and 5-(aminomethyl)-1-(2-hydroxycyclobutyl)-N,N-dimethyl-pyrazole-3-carboxamide (38.3 mg, 4.0 equiv, HCl) in DMF (1.5 mL) was added K₃PO₄ (37 mg, 5.0 equiv) at 25° C. The mixture was stirred at 60° C. for 6 hours. The residue was purified by prep-HPLC [C18, 0.1% formic acid condition] to afford the title compound (5.2 mg, 20.1% yield) as a white solid (0.33 formic acid salt). ¹H NMR (400 MHZ, METHANOL-d₄) δ 0.78 (br t, J=6.8 Hz, 3H) 1.74 (br t, J=9.2 Hz, 1H) 1.86-2.29 (m, 9H) 2.29-2.54 (m, 3H) 3.10 (s, 3H) 3.13-3.20 (m, 1H) 3.39 (s, 3H) 3.41 (br s, 2H) 4.35-4.62 (m, 3H) 4.92-5.16 (m, 3H) 5.35 (s, 1H) 6.71 (s, 1H) 7.04 (d, J=2.32 Hz, 1H) 7.22-7.32 (m, 2H) 7.68 (dd, J=9.2, 5.87 Hz, 1H) 8.50-8.53 (m, 1H) 9.19 (s, 1H); LCMS (ESI, M+1): m/z=731.3.

### Example 678

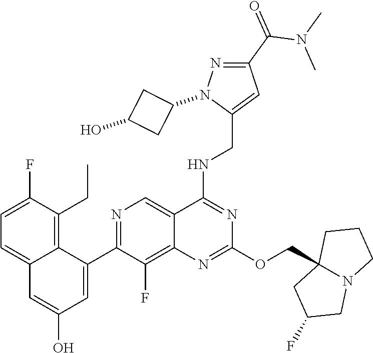

### 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-(cis-3-hydroxycyclobutyl)-N,N-dimethyl-1H-pyrazole-3-carboxamide

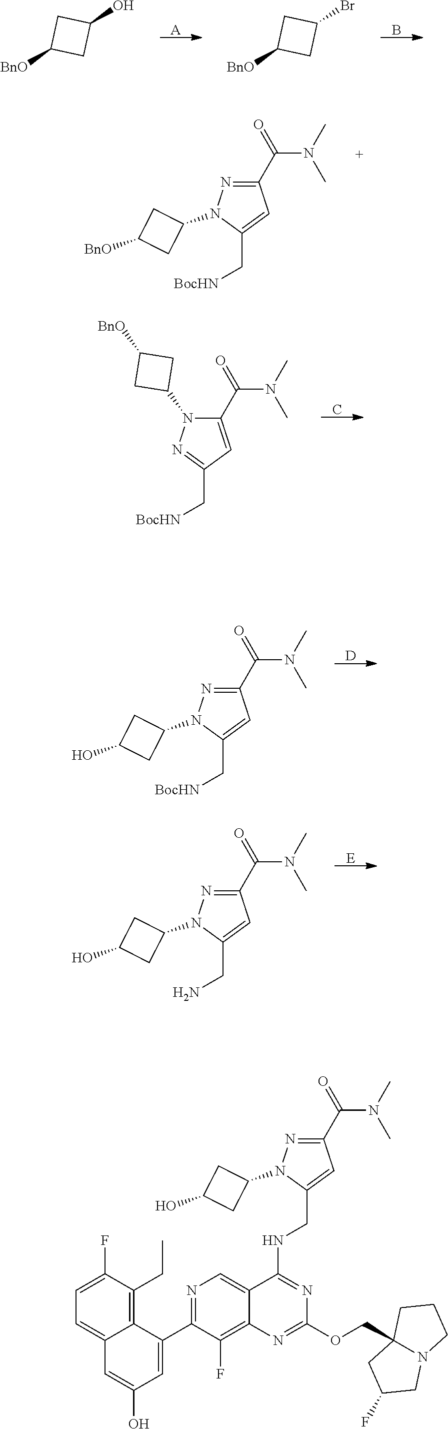

Step A. ((cis-3-bromocyclobutoxy)methyl)benzene: To a solution of 3-benzyloxycyclobutanol (1.0 g, 1 equiv), CBr₄ (4.5 g, 2.4 equiv) and TEA (1.3 g, 2.3 equiv) in DCM (10 mL) was added PPh₃ (3.4 g, 2.4 equiv) in DCM (5 mL) at 0° C. The mixture was stirred at 50° C. for 5 hours. The residue was poured into ice-NaHCO₃ (sat.) (20 mL) and stirred for 5 mins. The aqueous phase was extracted with DCM (50 mL×3). The combined organic phases were washed with brine (100 mL), dried with anhydrous Na₂SO₄, filtered and concentrated under vacuum. The residue was purified by silica gel chromatography (Petroleum ether/Ethyl acetate 1:0, 20:1) to afford the title compound (1.1 g, 81.3% yield) as a yellow liquid. ¹H NMR (400 MHZ, CHLOROFORM-d) δ=7.41-7.30 (m, 5H), 4.59-4.49 (m, 2H), 4.45 (s, 2H), 2.78-2.64 (m, 4H).

Step B. tert-butyl ((1-(cis-3-(benzyloxy)cyclobutyl)-3-(dimethylcarbamoyl)-1H-pyrazol-5-yl)methyl)carbamate and tert-butyl ((1-(cis-3-(benzyloxy)cyclobutyl)-5-(dimethylcarbamoyl)-1H-pyrazol-3-yl)methyl)carbamate: To a mixture of tert-butyl N-[[3-(dimethylcarbamoyl)-1H-pyrazol-5-yl]methyl]carbamate (500 mg, 1.0 equiv) and (3-bromocyclobutoxy)methylbenzene (674 mg, 1.5 equiv) in DMF (10 mL) was added Cs₂CO₃ (1.8 g, 3.0 equiv) at 25° C. under N₂. The mixture was stirred at 80° C. for 2 hours. The residue was poured into ice-water (20 mL) and stirred for 2 mins. The aqueous phase was extracted with ethyl acetate (30 mL×3). The combined organic phases were washed with brine (25 mL), dried with anhydrous Na₂SO₄, filtered, concentrated and purified with prep-TLC (SiO₂, Petroleum ether/Ethyl acetate 1:2) and SFC separation (column: DAICEL CHIRALPAK AD (250 mm×30 mm, 10 um); mobile phase: [0.1% NH₃H₂O ETOH]; B %: 25%-25%, 3.5 min) to afford the title compound tert-butyl ((1-(cis-3-(benzyloxy)cyclobutyl)-3-(dimethylcarbamoyl)-1H-pyrazol-5-yl)methyl)carbamate (385 mg, 47.2% yield) as yellow solid and tert-butyl ((1-(cis-3-(benzyloxy)cyclobutyl)-5-(dimethylcarbamoyl)-1H-pyrazol-3-yl)methyl)carbamate (193 mg, 23.0% yield) as yellow solid.

Step C. tert-butyl ((3-(dimethylcarbamoyl)-1-(cis-3-hydroxycyclobutyl)-1H-pyrazol-5-yl)methyl)carbamate: To a solution of tert-butyl ((1-(cis-3-(benzyloxy)cyclobutyl)-3-(dimethylcarbamoyl)-1H-pyrazol-5-yl)methyl)carbamate (385 mg, 1.0 equiv) in MeOH (10 mL) was added Pd/C (200 mg, 10% purity, 1.0 equiv) under N₂ at 25° C. The suspension was degassed under vacuum and purged with H₂ several times. The mixture was stirred under H₂ (15 Psi) at 40° C. for 2 hours. The reaction mixture was filtered and the filterate was concentrated to afford the title compound (193 mg, crude) as a white solid.

Step D. 5-(aminomethyl)-1-(cis-3-hydroxycyclobutyl)-N,N-dimethyl-1H-pyrazole-3-carboxamide: To a solution of tert-butyl ((3-(dimethylcarbamoyl)-1-(cis-3-hydroxycyclobutyl)-1H-pyrazol-5-yl)methyl)carbamate (152 mg, 1.0 equiv) in dioxane (3.0 mL) was added HCl/dioxane (4 M, 561 μL,5.0 equiv) at 25° C. The mixture was stirred at 25° C. for 1 hour. The reaction mixture was concentrated to afford the title compound (100 mg, crude, HCl) as a a white solid.

Step E. 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-(cis-3-hydroxycyclobutyl)-N,N-dimethyl-1H-pyrazole-3-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50 mg, 1.0 equiv) and 5-(aminomethyl)-1-(cis-3-hydroxycyclobutyl)-N,N-dimethyl-1H-pyrazole-3-carboxamide (46.4 mg, 2.0 equiv, HCl) in DMF (2.0 mL) was added K₃PO₄ (89.5 mg, 5.0 equiv) at 25° C. The mixture was stirred at 40° C. for 12 hours. The reaction mixture was diluted with DMF (5 mL), filtered and purified with prep-HPLC [C18, 0.1% formic acid condition] to afford the title compound (24.1 mg, 37.2% yield, 95.1% purity) as a white solid (0.33 formic acid salt). ¹H NMR (400 MHZ, METHANOL-d₄) δ=9.23-9.19 (m, 1H), 8.53 (br s, 1H), 7.70 (dd, J=5.8, 9.0 Hz, 1H), 7.33 (d, J=2.7 Hz, 1H), 7.27 (t, J=9.4 Hz, 1H), 7.06 (d, J=2.6 Hz, 1H), 6.72 (s, 1H), 5.50-5.31 (m, 1H), 4.99-4.92 (m, 2H), 4.69 (t, J=7.6 Hz, 1H), 4.49-4.37 (m, 2H), 4.16 (br t, J=7.2 Hz, 1H), 3.50-3.39 (m, 5H), 3.22-3.07 (m, 4H), 2.91-2.81 (m, 2H), 2.69-2.57 (m, 2H), 2.54-1.80 (m, 10H), 0.80 (t, J=7.3 Hz, 3H); LCMS (ESI, M+1): m/z=731.4.

### Example 679

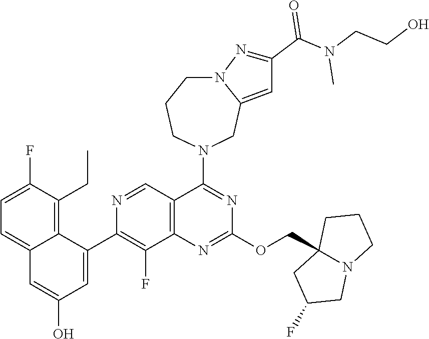 [SMILES: CCc1c(F)ccc2cc(O)cc(-c3ncc4c(N5CCCn6nc(C(=O)N(C)CCO)cc6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N-(2-hydroxyethyl)-N-methyl-5,6,7,8-tetrahydro-4H-pyrazolo[1,5-a][1,4]diazepine-2-carboxamide

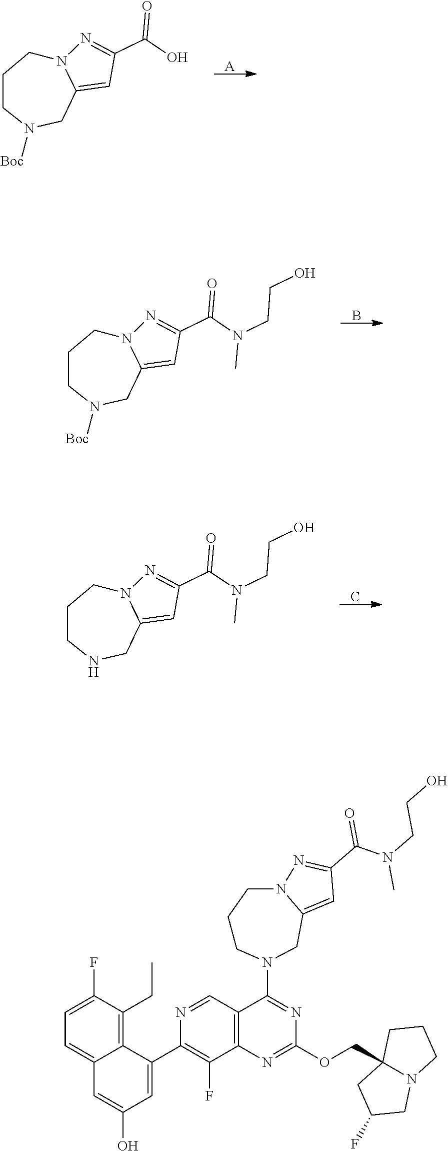

Step A: Tert-butyl 2-((2-hydroxyethyl)(methyl) carbamoyl)-7,8-dihydro-4H-pyrazolo[1,5-a][1,4]diazepine-5(6H)-carboxylate: To a solution of 5-(tert-butoxycarbonyl)-5,6,7,8-tetrahydro-4H-pyrazolo[1,5-a][1,4]diazepine-2-carboxylic acid (400 mg, 1 equiv) in dimethyl formamide (10 mL) was added dropwise triethylamine (287 mg, 395 μL, 2 equiv) and HATU (193 mg, 3.00 equiv) at 0° C. and the mixture was stirred for 0.5 hour. Then 2-(methylamino) ethanol (117 mg, 125 μL, 1.1 equiv) in tetramethylurea hexafluorophosphate (540 mg, 1 equiv) was added dropwise at 0° C. The resulting mixture was stirred at 25° C. for 1 hour. The reaction mixture was diluted with water (20 mL) and extracted with EtOAc (3×20 mL). The combined organic layers were washed with brine (3×15 mL), dried over anhydrous sodium sulfate, concentrated and purified with column choursomatography [SiO₂, petroleum ether/ethyl acetate 3:1 to 0:1] to afford the title compound (350 mg, 73% yield) as a colorless oil. LCMS (ESI, M+1): m/z=339.2

Step B: N-(2-hydroxyethyl)-N-methyl-5,6,7,8-tetrahydro-4H-pyrazolo[1,5-a][1,4]diazepine-2-carboxamide: To a solution of tert-butyl 2-((2-hydroxyethyl)(methyl) carbamoyl)-7,8-dihydro-4H-pyrazolo[1,5-a][1,4]diazepine-5(6H)-carboxylate (350 mg, 1 equiv) in DCM (3 mL) was added TFA (1 mL) at 0° C. and stirred for 0.5 hour. The reaction mixture was concentrated to give a residue. The residue was purified with prep-HPLC [column: Waters Xbridge 150×25 mm×5 um; mobile phase: [water (ammonia hydroxide v/v)-ACN]; B %: 2%-30%, 9 minutes] to afford the title compound (210 mg, 85% yield) as a white solid. LCMS (ESI, M+1): m/z=239.2

Step C: 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N-(2-hydroxyethyl)-N-methyl-5,6,7,8-tetrahydro-4H-pyrazolo[1,5-a][1,4]diazepine-2-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (90 mg, 1.0 equiv) and N-(2-hydroxyethyl)-N-methyl-5,6,7,8-tetrahydro-4H-pyrazolo[1,5-a][1,4]diazepine-2-carboxamide (181 mg, 5.0 equiv) in DMF (3 mL) was added K₃PO₄ (96.7 mg, 3.0 equiv). The mixture was stirred at 60° C. for 5 hours. The reaction mixture was concentrated and purified with prep-HPLC [column: Phenomenex Luna C18 150×25 mm×10 um; mobile phase: [water (FA)-ACN]; B %: 7%-37%, 10 minutes] to afford the title compound (12.8 mg, 11% yield) as a white solid (0.68 formic acid salt). ¹H NMR (400 MHZ, DMSO-d₆) δ=9.15-8.95 (m, 1H), 8.13 (s, 1H), 7.76 (dd, J=6.0, 9.0 Hz, 1H), 7.38-7.28 (m, 2H), 7.04 (d, J=2.4 Hz, 1H), 6.49 (br s, 1H), 5.44-5.20 (m, 1H), 4.89 (br d, J=4.0 Hz, 2H), 4.43-4.21 (m, 6H), 4.08 (br s, 3H), 2.96-2.87 (m, 3H), 2.68 (br s, 1H), 2.36-2.27 (m, 2H), 2.26-2.14 (m, 3H), 2.13-2.00 (m, 3H), 1.96-1.78 (m, 6H), 0.72 (t, J=7.2 Hz, 3H); LCMS (ESI, M+1): m/z=731.6.

### Example 680

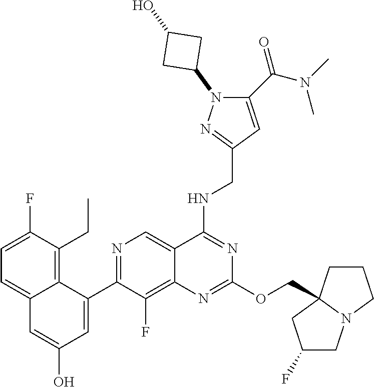

### 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-(trans-3-hydroxycyclobutyl)-N,N-dimethyl-1H-pyrazole-5-carboxamide

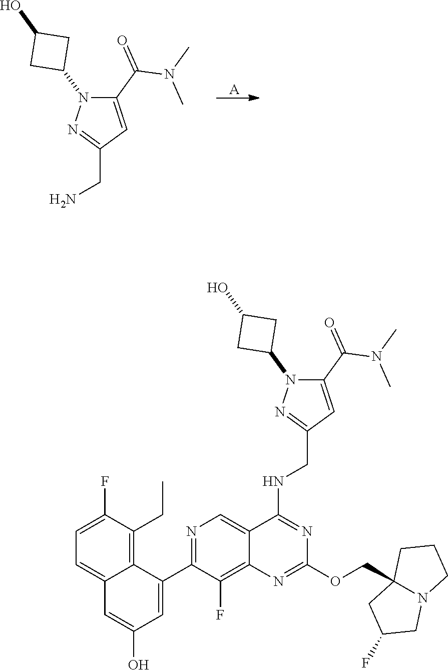

Step A. 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-(trans-3-hydroxycyclobutyl)-N,N-dimethyl-1H-pyrazole-5-carboxamide: The title compound was synthesized according to the procedure described for example 622 using 3-(aminomethyl)-1-(trans-3-hydroxycyclobutyl)-N,N-dimethyl-1H-pyrazole-5-carboxamide to produce the desired compound as a white solid (0.22 formic acid salt). ¹H NMR (400 MHZ, DMSO-d₆) δ=10.19-9.72 (m, 1H), 9.52 (br t, J=5.6 Hz, 1H), 9.34 (s, 1H), 8.17 (s, 1H), 7.76 (dd, J=6.0, 9.1 Hz, 1H), 7.39-7.29 (m, 2H), 6.99 (d, J=2.1 Hz, 1H), 6.50-6.44 (m, 1H), 5.37-5.18 (m, 1H), 5.08-4.95 (m, 1H), 4.88-4.67 (m, 2H), 4.49-4.38 (m, 1H), 4.19-4.12 (m, 1H), 4.06 (dd, J=1.6, 10.3 Hz, 1H), 3.08 (br d, J=5.8 Hz, 2H), 3.00 (br s, 1H), 2.96 (br d, J=5.2 Hz, 6H), 2.86-2.78 (m, 1H), 2.72-2.59 (m, 3H), 2.38-2.23 (m, 4H), 2.20-1.94 (m, 5H), 1.87-1.71 (m, 3H), 0.70 (br t, J=7.2 Hz, 3H); LCMS (ESI, M+1): m/z=731.1.

### Example 681

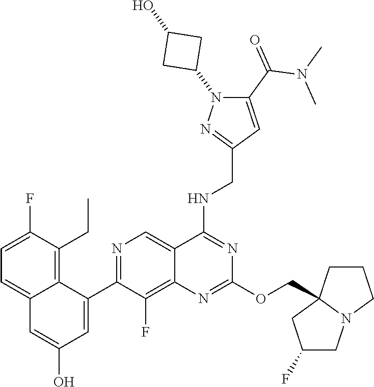

### 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-(cis-3-hydroxycyclobutyl)-N,N-dimethyl-1H-pyrazole-5-carboxamide

The title compound was synthesized from t-butyl ((1-(cis-3-(benzyloxy)cyclobutyl)-5-(dimethylcarbamoyl)-1H-pyrazol-3-yl)methyl)carbamate according to the 3-step procedure described in Steps C,D,E for Example 678 to produce the desired compound as a white solid (0.23 formic acid salt). ¹H NMR (400 MHZ, METHANOL-d4) δ=9.21 (s, 1H), 8.59-8.50 (m, 1H), 7.70 (dd, J=6.0, 9.1 Hz, 1H), 7.32 (d, J=2.6 Hz, 1H), 7.27 (t, J=9.4 Hz, 1H), 7.06 (d, J=2.4 Hz, 1H), 6.52 (s, 1H), 5.47-5.28 (m, 1H), 4.95 (s, 2H), 4.50-4.35 (m, 3H), 4.10 (t, J=6.8 Hz, 1H), 3.53-3.39 (m, 2H), 3.15-3.03 (m, 7H), 2.87-2.73 (m, 2H), 2.63-2.55 (m, 2H), 2.55-1.88 (m, 10H), 0.81 (t, J=7.3 Hz, 3H); LCMS (ESI, M+1): m/z=731.4.

### Example 682

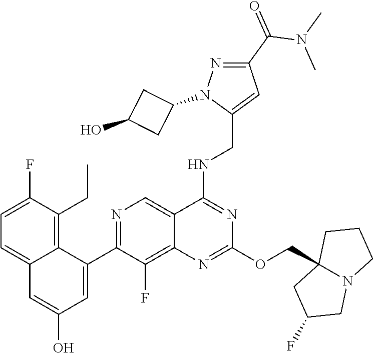 [SMILES: CCc1c(F)ccc2cc(O)cc(-c3ncc4c(NCc5cc(C(=O)N(C)C)nn5[C@H]5C[C@H](O)C5)nc(OC[C@@]56CCCN5C[C@H](F)C6)nc4c3F)c12]

### 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1-(trans-3-hydroxycyclobutyl)-N,N-dimethyl-1H-pyrazole-3-carboxamide

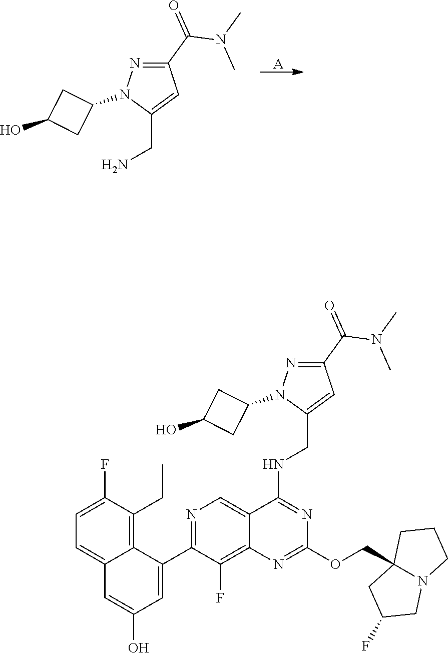

Step A. 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1-(trans-3-hydroxycyclobutyl)-N,N-dimethyl-1H-pyrazole-3-carboxamide: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (80 mg, 1.0 equiv) and 5-(aminomethyl)-1-(trans-3-hydroxycyclobutyl)-N,N-dimethyl-1H-pyrazole-3-carboxamide (66.8 mg, 1.8 equiv, HCl salt) in DMF (1 mL) were added 4 Å MS (50 mg, 1.0 equiv) and K₃PO₄ (143 mg, 5.0 equiv) drop-wise at 25° C. under N₂. The reaction was stirred at 40° C. for 12 hours. The reaction mixture was diluted with DMF (1 mL), filtered, and purified by prep-HPLC [C18, 0.1% formic acid condition] to afford the title compound (2.5 mg, 3% yield,) as yellow solid; ¹H NMR (400 MHZ, METHANOL-d₄) δ=9.30 (s, 1H), 8.57-8.52 (m, 1H), 7.72 (dd, J=5.9, 9.0 Hz, 1H), 7.34 (d, J=2.4 Hz, 1H), 7.29 (t, J=9.6 Hz, 1H), 7.08 (s, 1H), 6.71 (s, 1H), 6.03-5.93 (m, 1H), 5.44-5.23 (m, 2H), 4.46-4.36 (m, 2H), 4.08 (s, 2H), 3.48 (s, 3H), 3.26-3.18 (m, 3H), 3.16 (s, 3H), 3.13-3.02 (m, 4H), 2.53-1.85 (m, 9H), 0.80 (t, J=7.6 Hz, 3H); LCMS (ESI, M+1): m/z=731.0.

### Example 683

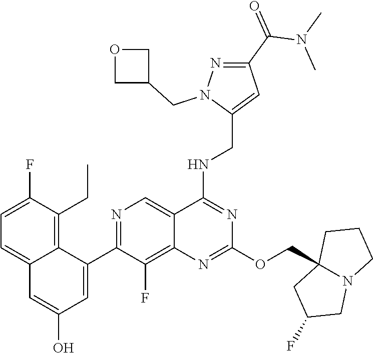 [SMILES: CCc1c(F)ccc2cc(O)cc(-c3ncc4c(NCc5cc(C(=O)N(C)C)nn5CC5COC5)nc(OC[C@@]56CCCN5C[C@H](F)C6)nc4c3F)c12]

### 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1-(oxetan-3-ylmethyl)-1H-pyrazole-3-carboxamide

### Example 684A

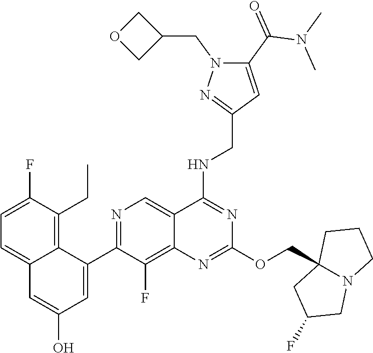 [SMILES: CCc1c(F)ccc2cc(O)cc(-c3ncc4c(NCc5cc(C(=O)N(C)C)n(CC6COC6)n5)nc(OC[C@@]56CCCN5C[C@H](F)C6)nc4c3F)c12]

### 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1-(oxetan-3-ylmethyl)-1H-pyrazole-5-carboxamide

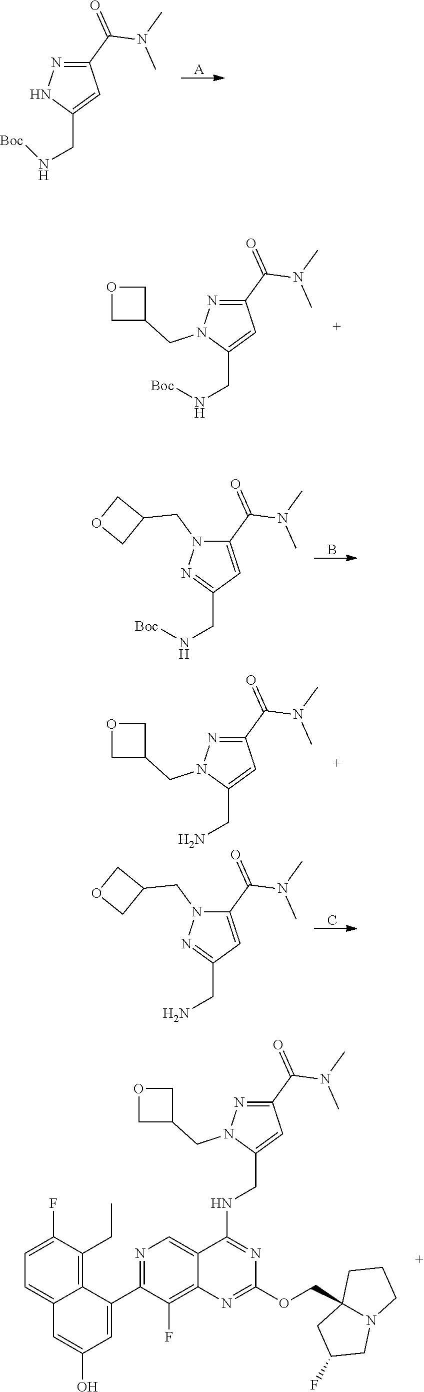

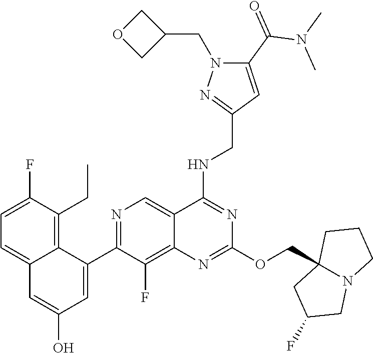 [SMILES: CCc1c(F)ccc2cc(O)cc(-c3ncc4c(NCc5cc(C(=O)N(C)C)n(CC6COC6)n5)nc(OC[C@@]56CCCN5C[C@H](F)C6)nc4c3F)c12]

Step A. tert-butyl ((3-(dimethylcarbamoyl)-1-(oxetan-3-ylmethyl)-1H-pyrazol-5-yl)methyl)carbamate and tert-butyl ((5-(dimethylcarbamoyl)-1-(oxetan-3-ylmethyl)-1H-pyrazol-3-yl)methyl)carbamate: To a mixture of tert-butyl ((5-(dimethylcarbamoyl)-1H-pyrazol-3-yl)methyl)carbamate (250 mg, 1 equiv) and 3-(bromomethyl) oxetane (281 mg, 2.0 equiv) in ACN (3 mL) was added Cs₂CO₃ (607 mg, 2.0 equiv). The reaction was stirred at 80° C. for 12 hours. The mixture was filtered and purified with prep-HPLC [Phenomenex luna C18 150×25 mm×10 μm; A: water (FA), B: ACN; B %: 20%-50% over 10 min] to afford the title compound (140 mg, 43% yield) as a yellow solid; LCMS (ESI, M+1): m/z=339.1.

Step B. 5-(aminomethyl)-N,N-dimethyl-1-(oxetan-3-ylmethyl)-1H-pyrazole-3-carboxamide and 3-(aminomethyl)-N,N-dimethyl-1-(oxetan-3-ylmethyl)-1H-pyrazole-5-carboxamide: To a mixture of tert-butyl N-[[5-(dimethylcarbamoyl)-2-(oxetan-3-ylmethyl) pyrazol-3-yl]methyl]carbamate (140 mg, 1.0 equiv) in DCM (229 μL) was added TFA (707 mg, 15 equiv). The reaction was stirred at 20° C. for 0.5 hour. The mixture was concentrated to afford the title compound (140 mg, TFA) as a yellow oil.

Step C. 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1-(oxetan-3-ylmethyl)-1H-pyrazole-3-carboxamide and 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1-(oxetan-3-ylmethyl)-1H-pyrazole-5-carboxamide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (40.0 mg, 1.0 equiv) and 5-(aminomethyl)-N,N-dimethyl-1-(oxetan-3-ylmethyl)-1H-pyrazole-3-carboxamide (47.6 mg, 2.0 equiv, TFA) in DMF (0.3 mL) and ACN (0.3 mL) were added 4 Å molecular sieves (20 mg, 1.0 equiv) and K₃PO₄ (43.0 mg, 3.0 equiv). The mixture was stirred at 40° C. for 36 hours. The mixture was filtered and purified with prep-HPLC [Phenomenex luna C18 150×25 mm×10 um; A: water (1.26FA), B: ACN; B %: 9%-39% over 10 min] followed by chiral SFC to afford Example 683 (3.82 mg, 7% yield) as an off-white solid; SFC: >99% ee, Chiralcel AD-3.50×4.6 mm I.D., 3 μm; Isocratic elution: 40% MeOH (0.05% DEA) in CO₂, 3 mL/min, 220 nm, t_{R}: 0.617 min; ¹H NMR (400 MHZ, methanol-d₄) δ=9.21 (s, 1H), 7.72-7.64 (m, 1H), 7.37-7.30 (m, 1H), 7.29-7.20 (m, 2H), 7.08-6.99 (m, 1H), 5.44-5.24 (m, 1H), 5.11-4.99 (m, 2H), 4.97-4.92 (m, 1H), 4.78-4.75 (m, 1H), 4.74-4.70 (m, 1H), 4.60-4.53 (m, 1H), 4.41-4.25 (m, 2H), 3.88-3.75 (m, 2H), 3.65-3.53 (m, 1H), 3.43-3.32 (m, 2H), 3.30-3.28 (m, 1H), 3.27-3.22 (m, 3H), 3.15-3.11 (m, 3H), 3.10-3.04 (m, 1H), 2.50-2.36 (m, 1H), 2.35-2.22 (m, 2H), 2.20-2.10 (m, 2H), 2.09-2.05 (m, 1H), 2.03-1.98 (m, 1H), 1.97-1.86 (m, 1H), 0.82-0.71 (m, 3H); ¹⁹F NMR (400 MHZ, methanol-d₄) δ=−121.115, −138.670, −173.966; LCMS (ESI, M+1): m/z=731.4. the and Example 684 (3.2 mg, 5% yield) as an off-white solid; SFC: >99% ee, Chiralcel OJ-3.50×4.6 mm I.D., 3 μm; Isocratic elution: 40% MeOH+ACN (0.05% DEA) in CO₂, 3 mL/min, 254 nm, t_{R}: 0.481 min; ¹H NMR (400 MHZ, methanol-d₄) δ=9.25-9.17 (m, 1H), 7.74-7.65 (m, 1H), 7.36-7.30 (m, 1H), 7.29-7.20 (m, 2H), 7.06-6.99 (m, 1H), 5.55-5.35 (m, 1H), 5.10-5.03 (m, 2H), 4.66-4.43 (m, 6H), 3.87-3.76 (m, 2H), 3.66-3.53 (m, 4H), 3.27-3.22 (m, 4H), 3.16-3.10 (m, 3H), 2.56-2.36 (m, 3H), 2.32-2.24 (m, 1H), 2.23-2.08 (m, 4H), 0.82-0.73 (m, 3H): ¹⁹F NMR (400 MHZ, methanol-d₄) δ=−76.918, −121.100, −174.161; LCMS (ESI, M+1): m/z=731.4.

### Example 685

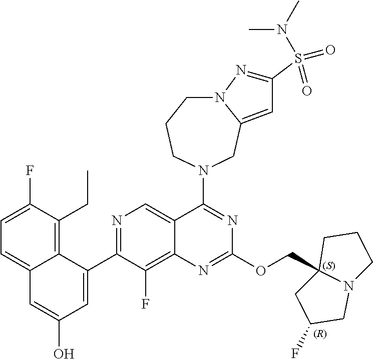

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-sulfonamide

The title compound was synthesized according to the 3-step procedure described for example 676 except for dimethylamine was used in Step A, to produce the desired compound as an off-white solid. ¹H NMR (400 MHZ, methanol-d₄) δ=9.17 (s, 1H), 7.67 (dd, J=6.0, 9.2 Hz, 1H), 7.30 (d, J=2.8 Hz, 1H), 7.25 (t, J=9.2 Hz, 1H), 7.04 (d, J=2.4 Hz, 1H), 6.90-6.83 (m, 1H), 5.41-5.15 (m, 3H), 4.62-4.52 (m, 2H), 4.51-4.37 (m, 2H), 4.33-4.19 (m, 2H), 3.39-3.32 (m, 1H), 3.28-3.17 (m, 2H), 3.09-2.98 (m, 1H), 2.77 (s, 6H), 2.54-2.40 (m, 3H), 2.36-2.25 (m, 1H), 2.24-2.12 (m, 3H), 2.06-1.91 (m, 3H), 0.83-0.74 (m, 3H); LCMS (ESI, M+1): m/z=737.4.

### Example 686

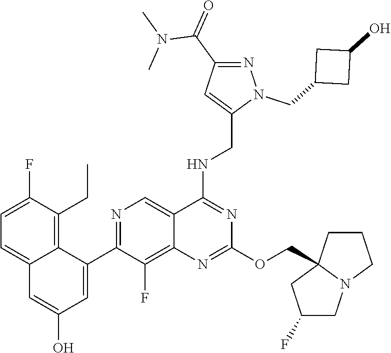

### 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-((trans-3-hydroxycyclobutyl)methyl)-N,N-dimethyl-1H-pyrazole-3-carboxamide

### Example 687

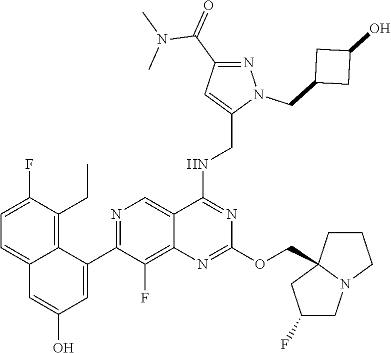

### 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-((cis-3-hydroxycyclobutyl)methyl)-N,N-dimethyl-1H-pyrazole-3-carboxamide

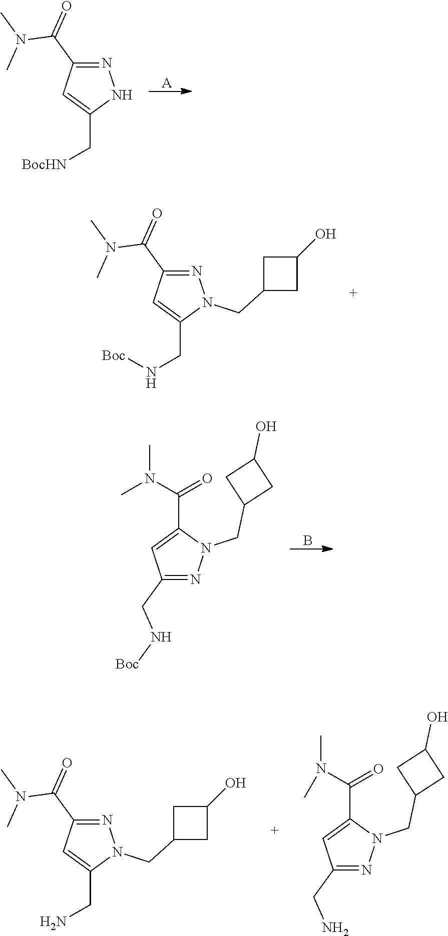

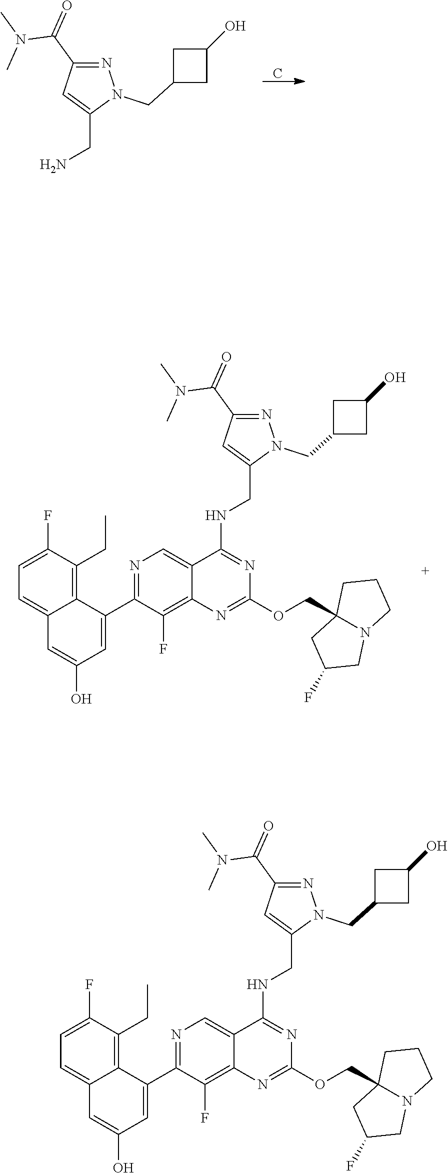

Step A. tert-butyl N-[[5-(dimethylcarbamoyl)-2-[(3-hydroxycyclobutyl)methyl]pyrazol-3-yl]methyl]carbamate and tert-butyl N-[[5-(dimethylcarbamoyl)-1-[(3-hydroxycyclobutyl)methyl]pyrazol-3-yl]methyl]carbamate: A mixture of tert-butyl N-[[3-(dimethylcarbamoyl)-1H-pyrazol-5-yl]methyl]carbamate (1 g, 1.0 equiv), (3-hydroxycyclobutyl)methyl 4-methylbenzenesulfonate (1.2 g, 1.2 equiv) and Cs₂CO₃ (2.4 g, 2.0 equiv) in DMF (10 mL) was degassed and purged with N₂ for 3 times, and then the mixture was stirred at 80° C. for 12 hours under N₂ atmosphere. The reaction mixture was diluted with H₂O (5 mL) and extracted with Ethyl acetate (4 mL×5). The combined organic layers were washed with brine (5 mL), dried over Na₂SO₄, filtered, concentrated and purified with prep-HPLC [C18, 0.1% formic acid condition] to afford the title compounds (340 mg, mixture, 23.2% yield) as a white solid. ¹H NMR (400 MHZ, CHLOROFORM-d) δ=8.04 (s, 1H), 8.08-7.99 (m, 1H), 6.60-6.26 (m, 1H), 5.08-4.79 (m, 2H), 4.38-4.26 (m, 3H), 4.18-4.10 (m, 2H), 3.37-3.04 (m, 6H), 2.83-2.68 (m, 1H), 2.49-2.35 (m, 1H), 2.32-2.20 (m, 1H), 1.77-1.61 (m, 1H), 1.47 (s, 9H); LCMS (ESI, M+1): m/z=353.2.

Step B. 5-(aminomethyl)-2-[(3-hydroxycyclobutyl)methyl]-N,N-dimethyl-pyrazole-3-carboxamide and 5-(aminomethyl)-1-[(3-hydroxycyclobutyl)methyl]-N,N-dimethyl-pyrazole-3-carboxamide: To the mixture of tert-butyl N-[[5-(dimethylcarbamoyl)-2-[(3-hydroxycyclobutyl)methyl]pyrazol-3-yl]methyl]carbamate and tert-butyl N-[[5-(dimethylcarbamoyl)-1-[(3-hydroxycyclobutyl)methyl]pyrazol-3-yl]methyl]carbamate (340 mg, 1.0 equiv) in dioxane (0.5 mL) was added HCl/dioxane (4 M, 2.4 mL, 20 equiv). The mixture was stirred at 20° C. for 4 hours. The reaction mixture was concentrated under reduced pressure to afford the title compounds (300 mg, crude mixture, HCl). The crude mixture (150 mg) was separated by SFC (column: DAICEL CHIRALPAK IC (250 mm*30 mm, 10 um); mobile phase: [0.1% NH₃H₂O MEOH]; B %: 35%-35%, 4.5 min) to afford 5-(aminomethyl)-1-((3-hydroxycyclobutyl)methyl)-N,N-dimethyl-1H-pyrazole-3-carboxamide (50 mg, 33.3% yield) and 3-(aminomethyl)-1-((3-hydroxycyclobutyl)methyl)-N,N-dimethyl-1H-pyrazole-5-carboxamide (100 mg, 66.7% yield).

5-(aminomethyl)-1-((3-hydroxycyclobutyl)methyl)-N,N-dimethyl-1H-pyrazole-3-carboxamide ¹H NMR (400 MHZ, METHANOL-d₄) δ=6.48-6.47 (m, 1H), 4.34-4.20 (m, 3H), 4.08-3.95 (m, 1H), 3.81 (s, 2H), 3.15-3.11 (m, 7H), 2.67 (qdd, J=4.2, 8.5, 12.7 Hz, 1H), 2.35-2.27 (m, 2H), 2.25-2.07 (m, 2H), 2.05-1.96 (m, 1H), 1.68-1.57 (m, 2H).

3-(aminomethyl)-1-((3-hydroxycyclobutyl)methyl)-N,N-dimethyl-1H-pyrazole-5-carboxamide ¹H NMR (400 MHZ, METHANOL-d₄) δ=6.64 (s, 1H), 4.20-4.14 (m, 5H), 4.02-3.92 (m, 1H), 3.25 (d, J=3.2 Hz, 3H), 3.00 (s, 3H), 2.70-2.59 (m, 1H), 2.33-2.06 (m, 4H), 2.02-1.90 (m, 1H), 1.66-1.55 (m, 2H)

Step C. 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-((trans-3-hydroxycyclobutyl)methyl)-N,N-dimethyl-1H-pyrazole-3-carboxamide and 5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-((cis-3-hydroxycyclobutyl)methyl)-N,N-dimethyl-1H-pyrazole-3-carboxamide: A mixture of 5-(aminomethyl)-1-((3-hydroxycyclobutyl)methyl)-N,N-dimethyl-1H-pyrazole-3-carboxamide (50.0 mg, 1.0 equiv), 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (111.5 mg, 0.95 equiv), K₃PO₄ (126 mg, 3 equiv) in DMF (1 mL) was degassed and purged with N₂ for 3 times, and then the mixture was stirred at 40 C for 4 hours. The reaction mixture was filtered, concentrated and purified with prep-HPLC [C18, 0.1% NH₃·H₂O condition] to afford the two title compounds EXAMPLE 686 (6.8 mg, 4.6% yield) and EXAMPLE 687 (3.6 mg, 2.4% yield) and the as white solids.

EXAMPLE 686 ¹H NMR (400 MHZ, METHANOL-d₄) δ=9.22-9.12 (m, 1H), 8.53 (s, 1H), 7.68 (dd, J=5.9, 9.0 Hz, 1H), 7.31 (d, J=2.6 Hz, 1H), 7.25 (t, J=9.4 Hz, 1H), 7.08-7.02 (m, 1H), 6.69 (s, 1H), 5.46-5.24 (m, 1H), 5.04-4.94 (m, 3H), 4.62 (s, 1H), 4.43-4.30 (m, 4H), 4.22-3.99 (m, 1H), 3.37-3.34 (m, 3H), 3.24-3.20 (m, 1H), 3.10-3.06 (m, 4H), 2.85-2.71 (m, 1H), 2.51-1.86 (m, 12H), 1.86-1.60 (m, 1H), 0.78 (t, J=7.2 Hz, 3H); ¹⁹F NMR (376 MHz, methanol-d4) δ=−121, −138, −173; LCMS (ESI, M+1): m/z=745.2.

EXAMPLE 687 ¹H NMR (400 MHZ, METHANOL-d₄) δ=9.23-9.13 (m, 1H), 7.69 (dd, J=5.6, 8.8 Hz, 1H), 7.32 (d, J=2.4 Hz, 1H), 7.26 (t, J=9.3 Hz, 1H), 7.04 (d, J=1.6 Hz, 1H), 6.65 (d, J=5.4 Hz, 1H), 5.71 (quin, J=6.4 Hz, 1H), 5.48 (br t, J=7.3 Hz, 1H), 5.41-5.22 (m, 1H), 4.74-4.50 (m, 1H), 4.42-4.31 (m, 4H), 4.06-4.00 (m, 2H), 3.37-3.34 (m, 4H), 3.21 (br s, 1H), 3.10 (d, J=5.6 Hz, 3H), 3.08-3.00 (m, 2H), 2.83-2.72 (m, 1H), 2.66-2.56 (m, 2H), 2.46-2.35 (m, 1H), 2.34-2.20 (m, 3H), 2.17-2.07 (m, 2H), 2.06-1.96 (m, 3H), 1.96-1.86 (m, 1H), 0.76 (t, J=7.3 Hz, 3H); ¹⁹F NMR (376 MHz, methanol-d4) δ=−121, −138, −173; LCMS (ESI, M+1): m/z=745.2.

### Example 688

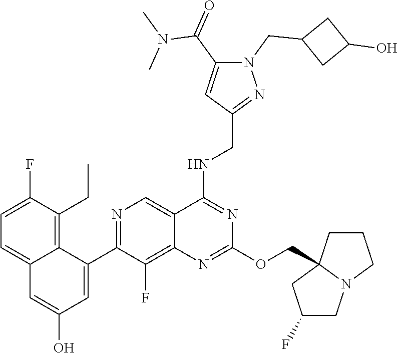 [SMILES: CCc1c(F)ccc2cc(O)cc(-c3ncc4c(NCc5cc(C(=O)N(C)C)n(CC6CC(O)C6)n5)nc(OC[C@@]56CCCN5C[C@H](F)C6)nc4c3F)c12]

### 3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-1-((3-hydroxycyclobutyl)methyl)-N,N-dimethyl-1H-pyrazole-5-carboxamide

The title compound was synthesized using 5-(aminomethyl)-2-[(3-hydroxycyclobutyl)methyl]-N,N-dimethyl-pyrazole-3-carboxamide according to the procedure described for example 622 to produce the desired compound as a yellow oil. ¹H NMR (400 MHZ, METHANOL-d₄) δ=9.17 (s, 1H), 8.55 (s, 1H), 7.67 (dd, J=5.6, 9.1 Hz, 1H), 7.30 (d, J=2.8 Hz, 1H), 7.25 (t, J=9.6 Hz, 1H), 7.04 (d, J=2.8 Hz, 1H), 6.58-6.56 (m, 1H), 5.39-5.21 (m, 1H), 4.90 (s, 2H), 4.32-4.23 (m, 4H), 4.01 (quin, J=7.6 Hz, 1H), 3.29-3.20 (m, 2H), 3.20-3.16 (m, 1H), 3.12-3.09 (m, 6H), 3.04-2.97 (m, 1H), 2.71-2.60 (m, 1H), 2.47 (td, J=7.2, 8.9 Hz, 1H), 2.36-2.08 (m, 8H), 2.04-1.93 (m, 3H), 1.93-1.84 (m, 1H), 1.66 (br d, J=2.8 Hz, 1H), 1.64-1.57 (m, 1H), 0.79 (t, J=7.2 Hz, 3H); LCMS (ESI, M+1): m/z=745.1.

### Example 689

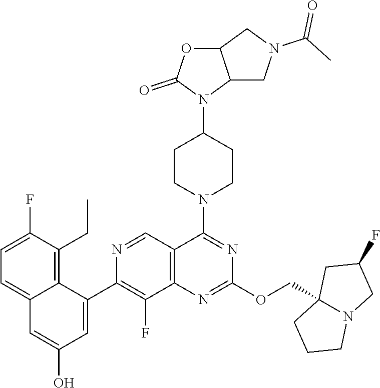 [SMILES: CCc1c(F)ccc2cc(O)cc(-c3ncc4c(N5CCC(N6C(=O)OC7CN(C(C)=O)CC76)CC5)nc(OC[C@@]56CCCN5C[C@H](F)C6)nc4c3F)c12]

### 5-acetyl-3-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperidin-4-yl)hexahydro-2H-pyrrolo[3,4-d]oxazol-2-one

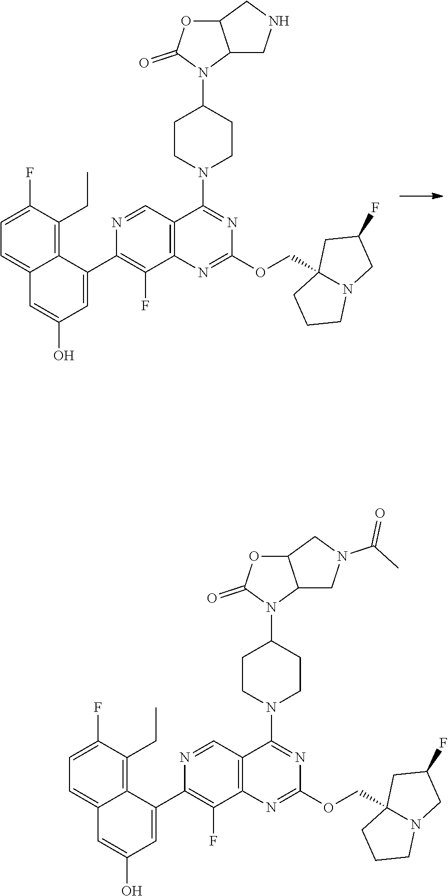 [SMILES: CCc1c(F)ccc2cc(O)cc(-c3ncc4c(N5CCC(N6C(=O)OC7CNCC76)CC5)nc(OC[C@@]56CCCN5C[C@H](F)C6)nc4c3F)c12 ; CCc1c(F)ccc2cc(O)cc(-c3ncc4c(N5CCC(N6C(=O)OC7CN(C(C)=O)CC76)CC5)nc(OC[C@@]56CCCN5C[C@H](F)C6)nc4c3F)c12]

A mixture of 3-(1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl) piperidin-4-yl)hexahydro-2H-pyrrolo[3,4-d]oxazol-2-one (15.0 mg, 1.0 equiv), TEA (6.50 mg, 3.0 equiv), acetyl chloride (2.00 mg, 1.2 equiv) in DCM (0.5 mL) was stirred at 0° C. for 2 hours under N₂ atmosphere. The mixture was concentrated and purified by prep-TLC (MeOH (10% NH₃·H₂O)/DCM 1:5) to afford the title compound (3.4 mg, 20.5% yield). 1H NMR (400 MHz, METHANOL-d₄) δ=9.00 (s, 1H), 7.58 (dd, J=5.9, 9.0 Hz, 1H), 7.21 (d, J=2.8 Hz, 1H), 7.15 (t, J=9.6 Hz, 1H), 6.96 (d, J=2.0 Hz, 1H), 5.42-5.17 (m, 1H), 5.12-4.93 (m, 1H), 4.54-4.24 (m, 3H), 4.04-3.57 (m, 4H), 3.56-3.23 (m, 6H), 3.18-2.98 (m, 2H), 2.59-1.73 (m, 13H), 1.29-1.08 (m, 4H), 0.81-0.63 (m, 3H); ¹⁹F NMR (377 MHZ, METHANOL-d4) δ=−121.09 (br s, 1F), −139.12 (br s, 1F), −173.88 (br s, 1F); LCMS (ESI, M+1): m/z=746.4.

### Example 690

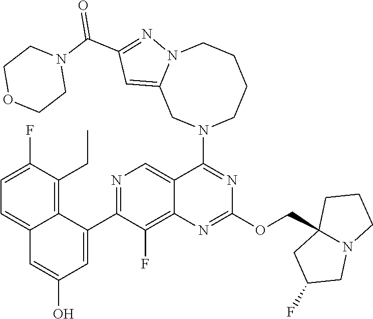 [SMILES: CCc1c(F)ccc2cc(O)cc(-c3ncc4c(N5CCCCn6nc(C(=O)N7CCOCC7)cc6C5)nc(OC[C@@]56CCCN5C[C@H](F)C6)nc4c3F)c12]

### (5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,5,6,7,8,9-hexahydropyrazolo[1,5-a][1,4]diazocin-2-yl)(morpholino)methanone

The title compound was synthesized according to the 3-step procedure described for example 671 to produce the desired compound as a yellow solid; ¹H NMR (400 MHZ, CD₃OD) δ=9.04 (s, 1H), 7.63 (dd, J=6.0, 8.8 Hz, 1H), 7.29-7.16 (m, 2H), 7.02 (d, J=2.4 Hz, 1H), 6.74 (s, 1H), 5.42-5.20 (m, 3H), 4.47 (br s, 2H), 4.31-4.19 (m, 2H), 4.15 (br s, 2H), 4.06 (br s, 2H), 3.79-3.66 (m, 6H), 3.19 (br d, J=19.2 Hz, 2H), 3.06-2.95 (m, 1H), 2.52-2.40 (m, 1H), 2.36-1.86 (m, 12H), 0.79 (t, J=7.2 Hz, 3H); ¹⁹F NMR (376 MHz, CD₃OD) δ=−122.10 (br s, 1F), −138.16 (s, 1F), −173.58 (s, 1F). LCMS (ESI, M+1): m/z=757.2

### Example 691

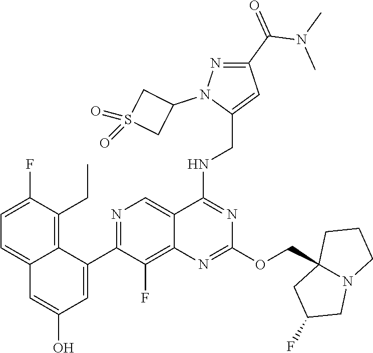 [SMILES: CCc1c(F)ccc2cc(O)cc(-c3ncc4c(NCc5cc(C(=O)N(C)C)nn5C5CS(=O)(=O)C5)nc(OC[C@@]56CCCN5C[C@H](F)C6)nc4c3F)c12]

### 1-(1,1-dioxidothietan-3-yl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide

### Example 692

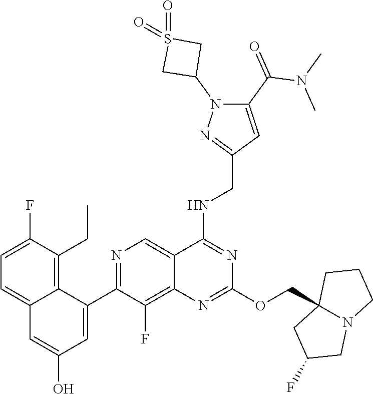 [SMILES: CCc1c(F)ccc2cc(O)cc(-c3ncc4c(NCc5cc(C(=O)N(C)C)n(C6CS(=O)(=O)C6)n5)nc(OC[C@@]56CCCN5C[C@H](F)C6)nc4c3F)c12]

### 1-(1,1-dioxidothietan-3-yl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide

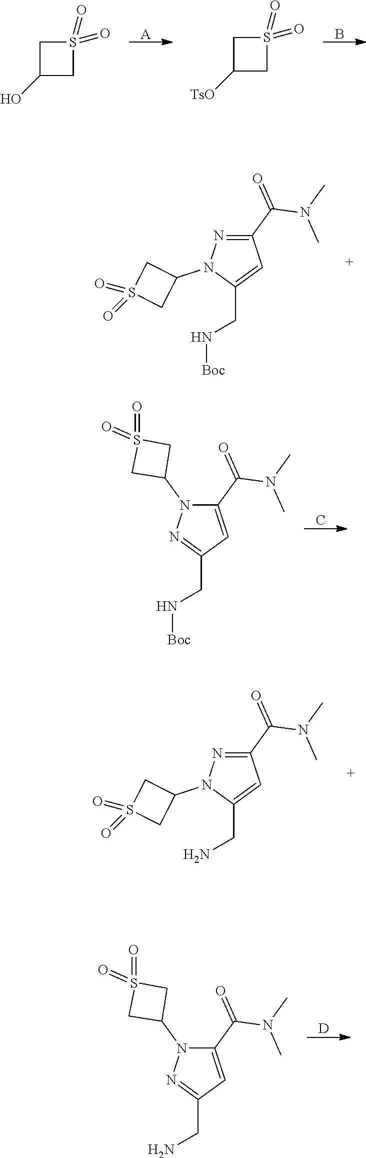

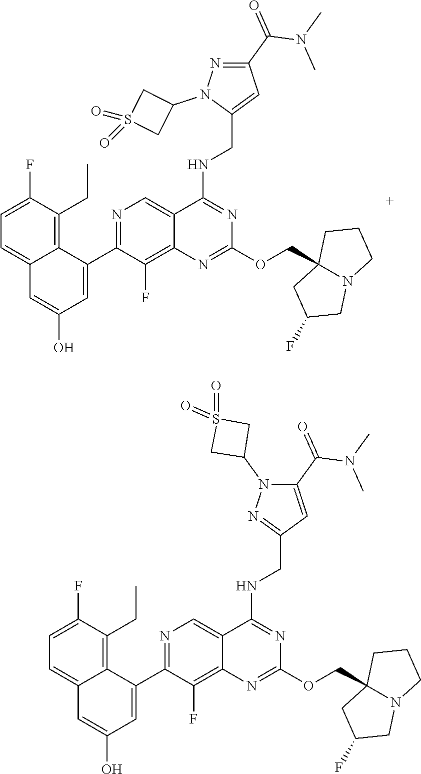 [SMILES: CCc1c(F)ccc2cc(O)cc(-c3ncc4c(NCc5cc(C(=O)N(C)C)nn5C5CS(=O)(=O)C5)nc(OC[C@@]56CCCN5C[C@H](F)C6)nc4c3F)c12 ; CCc1c(F)ccc2cc(O)cc(-c3ncc4c(NCc5cc(C(=O)N(C)C)n(C6CS(=O)(=O)C6)n5)nc(OC[C@@]56CCCN5C[C@H](F)C6)nc4c3F)c12]

Step A 1,1-dioxidothietan-3-yl 4-methylbenzenesulfonate: To a solution of 1,1-dioxothietan-3-ol (200 mg, 1.64 mmol, 1 eq) in THF (10 mL) was added NaH (130.99 mg, 3.27 mmol, 60% purity, 2 eq) at 0° C. and the resulting was stirred for 0.5 hr. Then TosCl (936.51 mg, 4.91 mol, 3 eq) was added and the mixture was stirred at 40° C. for 2.5 hr. The mixture was quenched with H₂O (1 mL), filtered and concentrated under reduced pressure to give a residue. The residue was purified by flash silica gel chromatography (ISCO®; 20 g SepaFlash® Silica Flash Column, Eluent of 0˜30% Ethyl acetate/Petroleum ethergradient @ 36 mL/min) to afford (1,1-dioxothietan-3-yl) 4-methylbenzenesulfonate (100 mg, 361.89 umol, 22.10% yield) as a white solid.

LCMS (ESI, M+1): m/z=277.0

Step B. tert-butyl ((3-(dimethylcarbamoyl)-1-(1,1-dioxidothietan-3-yl)-1H-pyrazol-5-yl)methyl)carbamate and tert-butyl ((5-(dimethylcarbamoyl)-1-(1,1-dioxidothietan-3-yl)-1H-pyrazol-3-yl)methyl)carbamate: A mixture of (1,1-dioxothietan-3-yl) 4-methylbenzenesulfonate (61.8 mg, 1 equiv), tert-butyl N-[[3-(dimethylcarbamoyl)-1H-pyrazol-5-yl]methyl]carbamate (60 mg, 1 equiv) and Cs₂CO₃ (219 mg, 3 equiv) in DMF (4.00 mL) was degassed and stirred at 60° C. for 2 hours under N₂ atmosphere. The mixture was extracted with EtOAc 90 mL (30 mL×3), dried over Na₂SO₄, filtered and concentrated under reduced pressure to give a residue. The residue was purified by prep-TLC (SiO₂, DCM/MeOH 10:1) to afford the title compound (50 mg, 54% yield) as a yellow solid. LCMS (ESI, M+1): m/z=373.1

Step C. 5-(aminomethyl)-1-(1,1-dioxidothietan-3-yl)-N,N-dimethyl-1H-pyrazole-3-carboxamide and 3-(aminomethyl)-1-(1,1-dioxidothietan-3-yl)-N,N-dimethyl-1H-pyrazole-5-carboxamide: A mixture of tert-butyl N-[[5-(dimethylcarbamoyl)-2-(1,1-dioxothietan-3-yl) pyrazol-3-yl]methyl]carbamate (50 mg, 1 equiv) in DCM (1.5 mL) and TFA (0.5 mL) was stirred at 10° C. for 1 hour under N₂ atmosphere. The mixture was concentrated under reduced pressure to give a residue. The residue was purified by prep-HPLC (FA condition; column: Waters Atlantis T3 150*30 mm*5 um; mobile phase: [water (FA)-ACN]; B %: 1%-20%, 10 min) to afford the title compound (25 mg, 68% yield) as a yellow solid. LCMS (ESI, M+1): m/z=273.1

Step D. 1-(1,1-dioxidothietan-3-yl)-5-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-3-carboxamide and 1-(1,1-dioxidothietan-3-yl)-3-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-N,N-dimethyl-1H-pyrazole-5-carboxamide: A mixture of regioisomeric 3- and 5-(aminomethyl)-1-(1,1-dioxothietan-3-yl)-N,N-dimethyl-pyrazole-3-carboxamide (18.4 mg, 2 equiv), 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (20.0 mg, 1 equiv), 4 Å molecular sieves (10 mg) and K₃PO₄ (71.7 mg, 10 equiv) in ACN (1.00 mL) and DMF (1.00 mL) was stirred at 60° C. for 2 hours. The mixture was concentrated under reduced pressure to give a residue. The residue was purified by prep-HPLC (neutral condition; column: Waters Xbridge 150*25 mm*5 um; mobile phase: [water (ammonia hydroxide v/v)-ACN]; B %: 32%-62%, 8.5 min) and following that separated by chiral SFC (condition: column: DAICEL CHIRALCEL OJ (250 mm×30 mm, 10 um); mobile phase: [0.1% NH₃H₂O ETOH]; B %: 40%-40%, 6.5 min) to afford the title compound EXAMPLE 691 (11.2 mg, 93% yield) as a yellow solid and EXAMPLE 692 (2.45 mg, 20% yield) as a yellow solid.

EXAMPLE 691 ¹H NMR (400 MHZ, METHANOL-d4) δ=9.15 (br s, 1H), 7.67 (dd, J=6.0, 9.2 Hz, 1H), 7.30 (d, J=2.4 Hz, 1H), 7.24 (t, J=9.6 Hz, 1H), 7.04 (br s, 1H), 6.77 (s, 1H), 5.83 (br s, 1H), 5.44-5.14 (m, 1H), 4.99-4.93 (m, 2H), 4.80 (br s, 3H), 4.36-4.24 (m, 2H), 3.41 (s, 3H), 3.26 (br d, J=10.3 Hz, 2H), 3.19 (br s, 1H), 3.10 (s, 3H), 3.06-2.98 (m, 1H), 2.49-2.38 (m, 1H), 2.37-2.28 (m, 1H), 2.25-2.20 (m, 1H), 2.18-2.08 (m, 2H), 2.03-1.95 (m, 2H), 1.92-1.82 (m, 1H), 1.34-1.27 (m, 1H), 0.77 (br t, J=7.2 Hz, 3H). LCMS (ESI, M+1): m/z=765.4

EXAMPLE 692 ¹H NMR (400 MHZ, METHANOL-d4) δ=9.19 (s, 1H), 7.67 (dd, J=2.8, 8.8 Hz, 1H), 7.31 (d, J=2.8 Hz, 1H), 7.25 (t, J=9.6 Hz, 1H), 7.04 (d, J=2.8 Hz, 1H), 6.67 (s, 1H), 5.50-5.44 (m, 1H), 4.95 (s, 2H), 4.79-4.70 (m, 2H), 4.67-4.58 (m, 4H), 4.49-4.34 (m, 2H), 3.46-3.36 (m, 3H), 3.35 (s, 1H), 3.15 (s, 3H), 3.09 (s, 3H), 2.48-2.43 (m, 1H), 2.36-2.30 (m, 1H), 2.20 (br d, J=8.7 Hz, 1H), 2.12-2.06 (m, 2H), 2.00-1.92 (m, 1H), 1.29 (s, 1H), 0.78 (t, J=7.2 Hz, 3H). LCMS (ESI, M+1): m/z=765.4

### Example 693

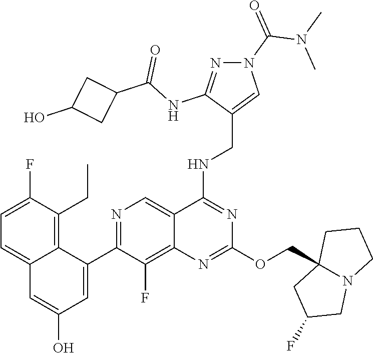 [SMILES: CCc1c(F)ccc2cc(O)cc(-c3ncc4c(NCc5cn(C(=O)N(C)C)nc5NC(=O)C5CC(O)C5)nc(OC[C@@]56CCCN5C[C@H](F)C6)nc4c3F)c12]

### 4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-3-(3-hydroxycyclobutanecarboxamido)-N,N-dimethyl-1H-pyrazole-1-carboxamide

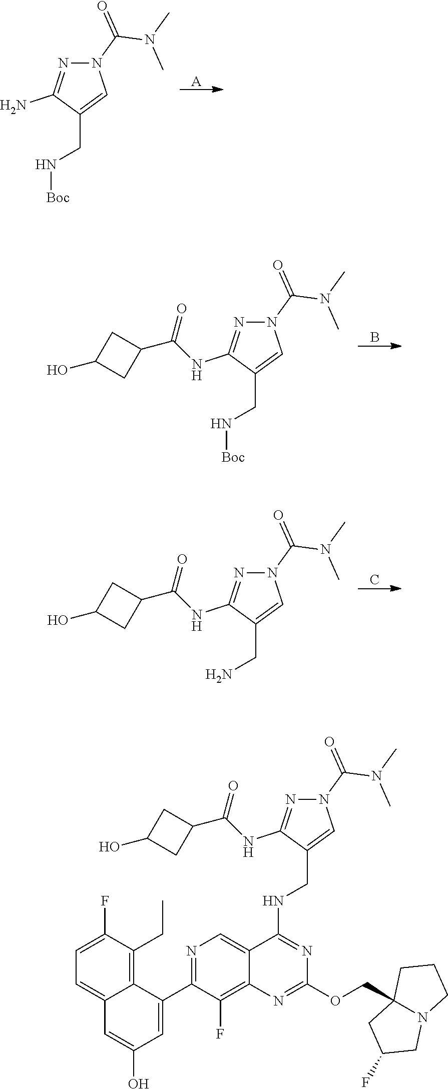

Step A. tert-butyl ((1-(dimethylcarbamoyl)-3-(3-hydroxycyclobutanecarboxamido)-1H-pyrazol-4-yl)methyl)carbamate: To a mixture of tert-butyl ((3-amino-1-(dimethylcarbamoyl)-1H-pyrazol-4-yl)methyl)carbamate (400 mg, 1.0 equiv) and 3-hydroxycyclobutanecarboxylic acid (245 mg, 1.5 equiv) in dichloromethane (4 mL) was added DIPEA (547 mg, 3.0 equiv) and HATU (805 mg, 1.5 equiv). The reaction was stirred at 20° C. for 2 hours. The mixture was diluted with water (20 ml) and extracted with EtOAc (2×30 mL). The combined organic layers were dried over anhydrous sodium sulfate, concentrated, and purified by reversed phase flash chromatography [C18, 0.1% formic acid condition] to afford the title compound (53 mg, 10% yield) as yellow solid; LCMS (ESI, M+1): m/z=382.0.

Step B. 4-(aminomethyl)-3-(3-hydroxycyclobutanecarboxamido)-N,N-dimethyl-1H-pyrazole-1-carboxamide: To a solution of tert-butyl ((1-(dimethylcarbamoyl)-3-(3-hydroxycyclobutanecarboxamido)-1H-pyrazol-4-yl)methyl)carbamate (43 mg, 1.0 equiv) in dichloromethane (0.5 mL) was added TFA (193 mg, 15 equiv). The reaction was stirred at 20° C. for 0.5 hour. The mixture was concentrated to afford the title compound (45 mg, TFA) as a yellow oil.

Step C. 4-(((7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-3-(3-hydroxycyclobutanecarboxamido)-N,N-dimethyl-1H-pyrazole-1-carboxamide: The title compound was synthesized according to the procedure described for example 541 to produce the desired compound as a white solid; SFC: Chiralcel OJ-3 50×4.6 mm I.D., 3 μm, Gradient elution: MeOH (0.05% DEA) in CO₂ from 5% to 40%, 3 mL/min; 220 nm, t_{R}: 1.593 min; ¹H NMR (400 MHZ, dimethylsulfoxide-d₆) δ=10.12-10.02 (m, 1H), 9.92 (br t, J=7.2 Hz, 1H), 9.29 (s, 1H), 9.20 (br d, J=4.4 Hz, 1H), 8.17 (d, J=3.6 Hz, 1H), 7.76 (dd, J=6.0, 9.6 Hz, 1H), 7.38-7.28 (m, 2H), 6.99 (d, J=2.4 Hz, 1H), 5.36-5.17 (m, 1H), 5.16-5.05 (m, 1H), 4.63-4.52 (m, 2H), 4.26-3.91 (m, 3H), 3.11 (br s, 6H), 3.09-2.99 (m, 3H), 2.86-2.78 (m, 1H), 2.35-2.25 (m, 3H), 2.20-2.08 (m, 2H), 2.06-1.97 (m, 4H), 1.96-1.67 (m, 4H), 0.71 (t, J=7.2 Hz, 3H); ¹⁹F NMR (377 MHz, dimethylsulfoxide-d₆) δ=−119.689, −139.615, −172.029; LCMS (ESI, M+1): m/z=774.6.

### Example 694

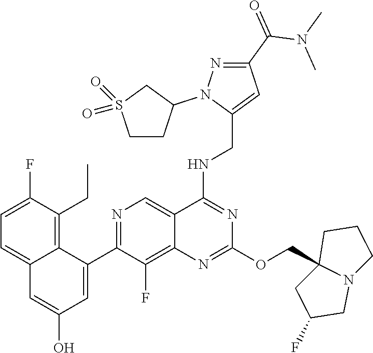 [SMILES: CCc1c(F)ccc2cc(O)cc(-c3ncc4c(NCc5cc(C(=O)N(C)C)nn5C5CCS(=O)(=O)C5)nc(OC[C@@]56CCCN5C[C@H](F)C6)nc4c3F)c12]

### 1-(1,1-dioxidotetrahydrothiophen-3-yl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide

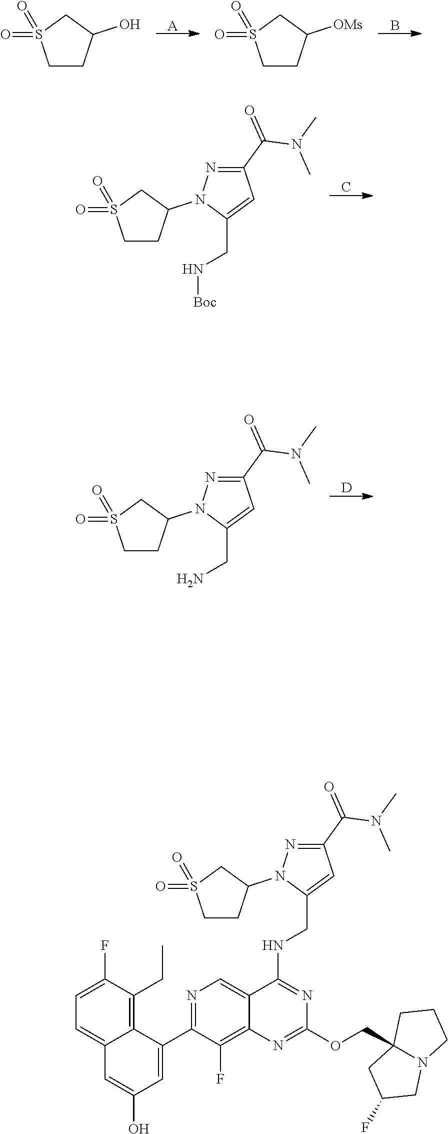

Step A. (1,1-dioxothiolan-3-yl) methanesulfonate: To a solution of 1,1-dioxothiolan-3-ol (400 mg, 1.0 equiv) and TEA (445.9 mg, 1.5 equiv) in DCM (10 mL) was added methanesulfonyl chloride (403.8 mg, 1.2 equiv) dropwise at 0° C. under N₂. The reaction mixture was warmed up to 25° C. and stirred for 2 hours. The reaction was quenched by iced water slowly and then extracted with DCM (25 Ml×3). The combined organic phases were washed with brine (30 mL), dried over anhydrous Na₂SO₄, filtered and concentrated in vacuo to afford the title compound (615 mg, crude) as an off-white solid.

Step B. tert-butyl N-[[5-(dimethylcarbamoyl)-1-(1,1-dioxothiolan-3-yl) pyrazol-3-yl]methyl]carbamate: To a solution of tert-butyl N-[[3-(dimethylcarbamoyl)-1H-pyrazol-5-yl]methyl]carbamate (260 mg, 1.0 equiv) and (1,1-dioxothiolan-3-yl) methanesulfonate (415.2 mg, 2.0 equiv) in DMF (3 mL) was added Cs₂CO₃ (947.2 mg. 3.0 equiv). The mixture was stirred at 40° C. for 4 hours. The residue was purified by prep-TLC (SiO₂, DCM/MeOH 10:1) to afford the title compound (350 mg, 85% yield) as a white solid.

Step C. 5-(aminomethyl)-1-(1,1-dioxothiolan-3-yl)-N,N-dimethyl-pyrazole-3-carboxamide: To a solution of tert-butyl N-[[5-(dimethylcarbamoyl)-1-(1,1-dioxothiolan-3-yl) pyrazol-3-yl]methyl]carbamate (380 mg, 1.0 equiv) in DCM (1.5 mL) was added TFA (770 mg, 6.9 equiv). The mixture was stirred at 25° C. for 1 hour. The residue was concentrated and purified by prep-HPLC [C18, 0.1% NH₃·H₂O condition], which was further separated by SFC to afford the title compound (72 mg, 25% yield) as a white solid. ¹H NMR (400 MHZ, METHANOL-d₄) δ 2.60-2.75 (m, 2H) 3.09 (s, 3H) 3.19-3.27 (m, 1H) 3.37 (s, 3H) 3.48-3.57 (m, 2H) 3.68 (dd, J=13.6, 8.44 Hz, 1H) 3.92 (s, 2H) 5.34-5.46 (m, 1H) 6.61 (s, 1H).

Step D. 1-(1,1-dioxidotetrahydrothiophen-3-yl)-5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1H-pyrazole-3-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (12 mg, equiv) and 5-(aminomethyl)-1-(1,1-dioxothiolan-3-yl)-N,N-dimethyl-pyrazole-3-carboxamide (23.2 mg, 4.0 eq) in DMF (0.5 mL) was added K₃PO₄ (21.5 mg, 5.0 equiv) at 25° C. The mixture was stirred at 40° C. for 12 hours before being filtered and concentrated. The residue was purified by prep-HPLC [C18, 0.1% formic acid condition] to afford the title compound (7.5 mg, 46.6% yield) as a white solid (0.39 formic acid salt). ¹H NMR (400 MHZ, METHANOL-d₄) δ 0.71-0.85 (m, 3H) 1.30 (br s, 1H) 1.95-2.34 (m, 6H) 2.36-2.56 (m, 3H) 2.66-2.81 (m, 2H) 3.10 (s, 3H) 3.40 (s, 2H) 3.53-3.80 (m, 6H) 4.48-4.67 (m, 5H) 5.03-5.10 (m, 1H) 5.34-5.54 (m, 1H) 5.63-5.77 (m, 1H) 6.77 (s, 1H) 7.01 (s, 1H) 7.25 (t, J=9.6 Hz, 1H) 7.29-7.36 (m, 1H) 7.64-7.72 (m, 1H) 8.48 (br d, J=6.8 Hz, 1H) 9.17-9.23 (m, 1H); LCMS (ESI, M+1): m/z=779.4.

### Example 695

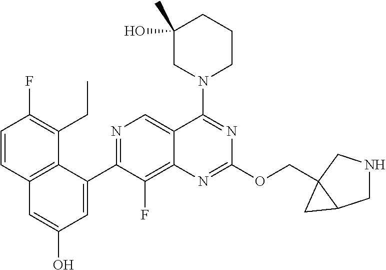 [SMILES: CCc1c(F)ccc2cc(O)cc(-c3ncc4c(N5CCC[C@@](C)(O)C5)nc(OCC56CNCC5C6)nc4c3F)c12]

### (3R)-1-(2-((3-azabicyclo[3.1.0]hexan-1-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

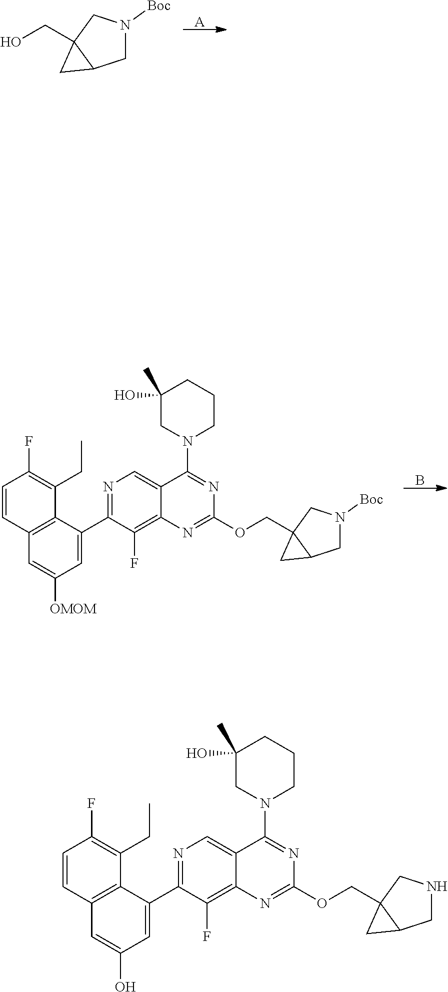

Step A. tert-butyl 1-(((7-(8-ethyl-7-fluoro-3-(methoxymethoxy)naphthalene-1-yl)-8-fluoro-4-((R)-3-hydroxy-3-methylpiperidin-1-yl)pyrido[4,3-d]pyrimidin-2-yl)oxy)methyl)-3-azabicyclo[3.1.0]hexane-3-carboxylate: To a solution of tert-butyl 1-(hydroxymethyl)-3-azabicyclo[3.1.0]hexane-3-carboxylate (200 mg, 2.2 equiv) and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (225 mg, 1.0 equiv) in THF (4.00 mL) was added t-BuOK (1.07 mL, 2.50 equiv). The mixture was stirred at 60° C. for 12 hours. The reaction mixture was quenched by addition of water (20.0 mL) at 20° C., and extracted with ethyl acetate (30.0 mL×3). The combined organic layers were washed with brine (20.0 mL), dried over anhydrous sodium sulfate, filtered, concentrated and purified by prep-TLC (SiO₂, DCM/MeOH 10:1) to afford the title compound (220 mg, 61% yield) as a yellow solid; LCMS (ESI, M+1): m/z=706.4.

Step B. (3R)-1-(2-((3-azabicyclo[3.1.0]hexan-1-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a solution of tert-butyl 1-(((7-(8-ethyl-7-fluoro-3-(methoxymethoxy)naphthalene-1-yl)-8-fluoro-4-((R)-3-hydroxy-3-methylpiperidin-1-yl)pyrido[4,3-d]pyrimidin-2-yl)oxy)methyl)-3-azabicyclo[3.1.0]hexane-3-carboxylate (50.0 mg, 1.0 equiv) in HCl/MeOH (2.00 mL). The mixture was stirred at 0° C. for 1 hour. The reaction mixture was concentrated and purified with prep-HPLC (column: Phenomenex luna C18 150×25 mm×10 um; mobile phase: [water (FA)-ACN]; B %: 16%-46%, 10 min) to afford the title compound (7.13 mg, 18% yield) as a white solid (0.76 formic acid salt); ¹H NMR (400 MHZ, METHANOL-d4) δ=9.24 (s, 1H), 8.55 (s, 1H), 7.74-7.64 (m, 1H), 7.33 (d, J=2.4 Hz, 1H), 7.27 (t, J=9.6 Hz, 1H), 7.07 (s, 1H), 4.75-4.53 (m, 4H), 4.39-4.26 (m, 1H), 3.72-3.56 (m, 1H), 3.54-3.39 (m, 4H), 2.58-2.40 (m, 1H), 2.30-2.11 (m, 2H), 1.96-1.73 (m, 4H), 1.31 (d, J=10.0 Hz, 3H), 1.17-1.07 (m, 1H), 0.83 (q, J=7.2 Hz, 4H); ¹⁹F NMR (376 MHz, METHANOL-d4) δ=−121.15 (br d, J=11.2 Hz, 1F), −135.57-−143.88 (m, 1F); LCMS (ESI, M+1): m/z=562.3.

### Example 696

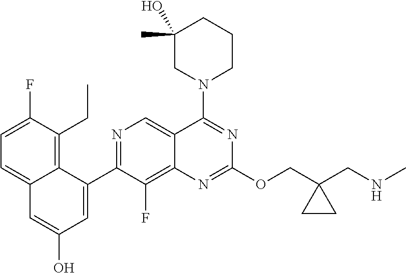 [SMILES: CCc1c(F)ccc2cc(O)cc(-c3ncc4c(N5CCC[C@@](C)(O)C5)nc(OCC5(CNC)CC5)nc4c3F)c12]

### (R)-1-(7-(8-ethyl-7-fluoro-3-hydroxynaphthalen-1-yl)-8-fluoro-2-((1-((methylamino)methyl)cyclopropyl)methoxy)pyrido[4,3-d]pyrimidin-4-yl)-3-methylpiperidin-3-ol

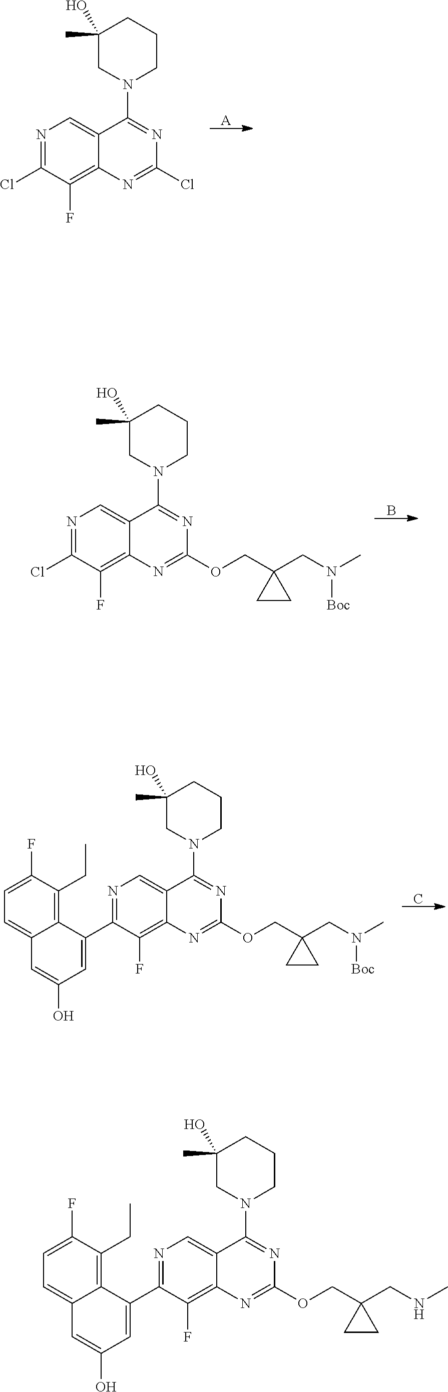

Step A: (R)-tert-butyl ((1-(((7-chloro-8-fluoro-4-(3-hydroxy-3-methylpiperidin-1-yl)pyrido[4,3-d]pyrimidin-2-yl)oxy)methyl)cyclopropyl)methyl)(methyl)carbamate: To a solution of (R)-1-(2,7-dichloro-8-fluoropyrido[4,3-d]pyrimidin-4-yl)-3-methylpiperidin-3-ol (2 g, 1.0 equiv) and tert-butyl ((1-(hydroxymethyl)cyclopropyl)methyl)(methyl)carbamate (1.82 g, 1.4 equiv) in DMSO (10 mL) was added CsF (4.56 g, 5.0 equiv). The mixture was stirred at 100° C. for 2 hours. The mixture was diluted with H₂O (100 mL), extracted with ethyl acetate (4×20 mL). The combined organic layers were dried over Na₂SO₄, concentrated and purified by reversed phase flash chromatography [C18, 0.1% formic acid condition] to afford the title compound (1.1 g, 23% yield) as a yellow solid; LCMS (ESI, M+1): m/z=510.3.

Step B: (R)-tert-butyl ((1-(((7-(8-ethyl-7-fluoro-3-hydroxynaphthalen-1-yl)-8-fluoro-4-(3-hydroxy-3-methylpiperidin-1-yl)pyrido[4,3-d]pyrimidin-2-yl)oxy)methyl)cyclopropyl)methyl)(methyl)carbamate: A mixture of (R)-tert-butyl ((1-(((7-chloro-8-fluoro-4-(3-hydroxy-3-methylpiperidin-1-yl)pyrido[4,3-d]pyrimidin-2-yl)oxy)methyl)cyclopropyl)methyl)(methyl)carbamate (110 mg, 1.0 equiv), 5-ethyl-6-fluoro-4-(4,4,5,5-tetramethyl-1,3,2-dioxaborolan-2-yl)naphthalen-2-ol (88.6 mg, 1.3 equiv), CataCXium A Pd G3 (15.8 mg, 0.1 equiv) and K₃PO₄ (1.5 M, 3.0 equiv) in methoxycyclopentane (1.0 mL) was degassed and stirred at 90° C. for 2 hours under N₂ atmosphere. The mixture was diluted with H₂O (5 mL), extracted with ethyl acetate (3×5 mL). The combined organic layer was dried over Na₂SO₄, concentrated and purified by reversed phase flash chromatography [C18, 0.1% NH₃·H₂O condition] to afford the title compound (20 mg, 13% yield,) as a yellow oil. LCMS (ESI, M+1): m/z=664.3.

Step C: (R)-1-(7-(8-ethyl-7-fluoro-3-hydroxynaphthalen-1-yl)-8-fluoro-2-((1-((methylamino)methyl)cyclopropyl)methoxy)pyrido[4,3-d]pyrimidin-4-yl)-3-methylpiperidin-3-ol: To a solution of (R)-tert-butyl ((1-(((7-(8-ethyl-7-fluoro-3-hydroxynaphthalen-1-yl)-8-fluoro-4-(3-hydroxy-3-methylpiperidin-1-yl)pyrido[4,3-d]pyrimidin-2-yl)oxy)methyl)cyclopropyl)methyl)(methyl)carbamate (100 mg, 1.0 equiv) in DCM (0.5 mL) was added 2,6-dimethylpyridine (161 mg, 175 μL, 10.0 equiv) and trimethylsilyl trifluoromethanesulfonate (335 mg, 10 equiv) at 0° C. The reaction solution was stirred at 20° C. for 0.5 hour. The solution was diluted with water (5 mL) and extracted with ethyl acetate (3×5 mL). The combined organic layer was dried over Na₂SO₄, concentrated and purified by prep HPLC (Phenomenex Synergi, C18 150×25 mm×10 μm, A: water (FA), B: ACN, B %: 15%-45% over 10 min) to afford the title compound (19.6 mg, 0.85 formic acid salt, 21% yield) as an off-white solid; ¹H NMR (400 MHZ, METHANOL-d₄) δ=9.25 (s, 1H), 7.68 (dd, J=6.0, 9.2 Hz, 1H), 7.32 (d, J=2.8 Hz, 1H), 7.26 (t, J=9.2 Hz, 1H), 7.06 (s, 1H), 4.57 (br d, J=12.4 Hz, 1H), 4.47-4.38 (m, 2H), 4.36-4.27 (m, 1H), 3.67-3.55 (m, 1H), 3.47-3.37 (m, 1H), 3.20-3.10 (m, 2H), 2.72 (s, 3H), 2.53-2.40 (m, 1H), 2.27-2.08 (m, 2H), 1.91-1.73 (m, 3H), 1.29 (d, J=8.8 Hz, 3H), 0.93-0.88 (m, 2H), 0.87-0.84 (m, 2H), 0.84-0.76 (m, 3H); LCMS (ESI, M+1): m/z=564.4.

### Example 697

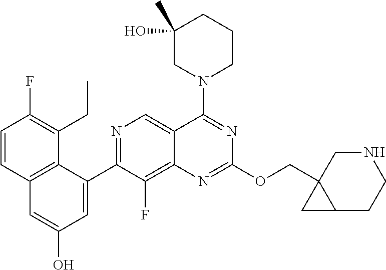 [SMILES: CCc1c(F)ccc2cc(O)cc(-c3ncc4c(N5CCC[C@@](C)(O)C5)nc(OCC56CNCCC5C6)nc4c3F)c12]

### (3R)-1-(2-((3-azabicyclo[4.1.0]heptan-1-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

The title compound was synthesized from tert-butyl 1-(hydroxymethyl)-3-azabicyclo[4.1.0]heptane-3-carboxylate according to the 2-step procedure described for example 695 to produce the desired compound as a white solid (0.66 formic acid salt). ¹H NMR (400 MHZ, CD₃OD) δ=9.22 (s, 1H), 8.55 (s, 1H), 7.68 (dd, J=5.6, 8.8 Hz, 1H), 7.35-7.20 (m, 2H), 7.08-7.02 (m, 1H), 4.55 (br d, J=12.8 Hz, 1H), 4.47-4.39 (m, 1H), 4.35-4.23 (m, 2H), 3.72-3.54 (m, 2H), 3.48-3.39 (m, 1H), 3.24 (br d, J=13.6 Hz, 1H), 3.09-2.98 (m, 1H), 2.77 (m, 1H), 2.47 (m, 1H), 2.32-2.09 (m, 3H), 1.97-1.73 (m, 4H), 1.41-1.33 (m, 1H), 1.29 (d, J=10.0 Hz, 3H), 1.05 (dd, J=5.6, 9.2 Hz, 1H), 0.88-0.74 (m, 4H); ¹⁹F NMR (377 MHz, CD₃OD) δ=−121.17 (s, 1F), −139.53 (s, 1F). LCMS (ESI, M+1): m/z=576.2.

### Example 698

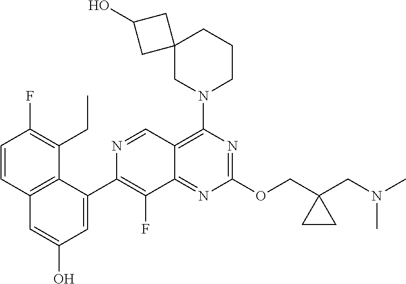 [SMILES: CCc1c(F)ccc2cc(O)cc(-c3ncc4c(N5CCCC6(CC(O)C6)C5)nc(OCC5(CN(C)C)CC5)nc4c3F)c12]

### 6-(2-((1-((dimethylamino)methyl)cyclopropyl)methoxy)-7-(8-ethyl-7-fluoro-3-hydroxynaphthalen-1-yl)-8-fluoropyrido[4,3-d]pyrimidin-4-yl)-6-azaspiro[3.5]nonan-2-ol

The title compound was synthesized according to the 3-step procedure described for example 585 to produce the desired compound as a white solid (0.24FA). ¹H NMR (400 MHZ, METHANOL-d₄) δ=9.07 (d, J=5.8 Hz, 1H), 7.68-7.66 (m, 1H), 7.31 (d, J=2.6 Hz, 1H), 7.25 (t, J=9.6 Hz, 1H), 7.07 (t, J=2.4 Hz, 1H), 4.50-4.38 (m, 2H), 4.33-4.21 (m, 1H), 4.18-4.05 (m, 2H), 4.04-3.90 (m, 2H), 2.94-2.80 (m, 2H), 2.63-2.62 (m, 6H), 2.54-2.44 (m, 1H), 2.34-2.14 (m, 3H), 1.87-1.67 (m, 6H), 0.87-0.78 (m, 5H), 0.70 (s, 2H); LCMS (ESI, M+1): m/z=604.3.

### Example 699

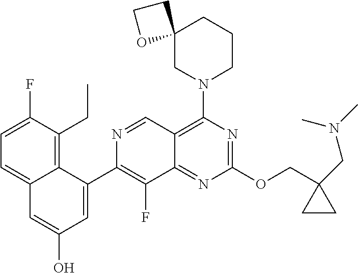 [SMILES: CCc1c(F)ccc2cc(O)cc(-c3ncc4c(N5CCC[C@]6(CCO6)C5)nc(OCC5(CN(C)C)CC5)nc4c3F)c12]

### (S)-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-5-ethyl-6-fluoronaphthalen-2-ol

### Example 700

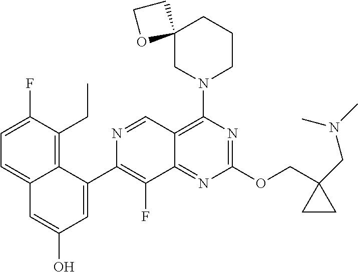 [SMILES: CCc1c(F)ccc2cc(O)cc(-c3ncc4c(N5CCC[C@@]6(CCO6)C5)nc(OCC5(CN(C)C)CC5)nc4c3F)c12]

### (R)-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-5-ethyl-6-fluoronaphthalen-2-ol

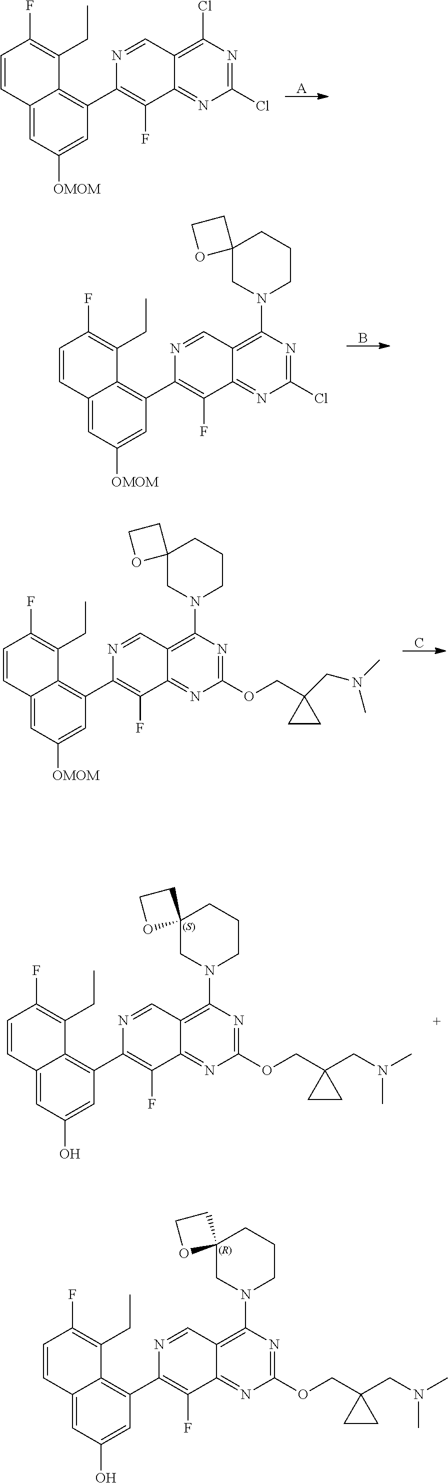

Step A. 6-(2-chloro-7-(8-ethyl-7-fluoro-3-(methoxymethoxy)naphthalen-1-yl)-8-fluoropyrido[4,3-d]pyrimidin-4-yl)-1-oxa-6-azaspiro[3.5]nonane: To a solution of 2,4-dichloro-7-[8-ethyl-7-fluoro-3-(methoxymethoxy)-1-naphthyl]-8-fluoro-pyrido[4,3-d]pyrimidine (500 mg, 1.0 equiv) and DIEA (717 mg, 5.0 equiv) in DCM (5.0 mL) was added 1-oxa-8-azaspiro[3.5]nonane (241 mg, 1.0 equiv). The reaction mixture was stirred at 25° C. for 1 hour under N₂ atmosphere. The mixture was concentrated under reduced pressure and purified with prep-HPLC [C18, 0.1% formic acid condition] to afford the title compound (130 mg, 22% yield) as white solid; ¹H NMR (400 MHZ, CHLOROFORM-d) δ=9.36 (s, 1H), 7.70 (dd, J=6.0, 9.2 Hz, 1H), 7.61-7.47 (m, 1H), 7.31-7.27 (m, 1H), 7.22 (s, 1H), 5.60-5.05 (m, 2H), 4.81-4.51 (m, 3H), 4.49-4.31 (m, 1H), 3.78 (dd, J=13.6, 18.4 Hz, 1H), 3.58-3.39 (m, 4H), 2.62-2.43 (m, 3H), 2.39-2.28 (m, 1H), 2.24-2.02 (m, 2H), 1.99-1.75 (m, 2H), 0.85 (q, J=7.2 Hz, 3H).

Step B. 1-(1-(((7-(8-ethyl-7-fluoro-3-(methoxymethoxy)naphthalen-1-yl)-8-fluoro-4-(1-oxa-6-azaspiro[3.5]nonan-6-yl)pyrido[4,3-d]pyrimidin-2-yl)oxy)methyl)cyclopropyl)-N,N-dimethylmethanamine: A mixture of 8-[2-chloro-7-[8-ethyl-7-fluoro-3-(methoxymethoxy)-1-naphthyl]-8-fluoro-pyrido[4,3-d]pyrimidin-4-yl]-1-oxa-8-azaspiro[3.5]nonane (60.0 mg, 1.0 equiv) and [1-[(dimethylamino)methyl]cyclopropyl]methanol (400 mg, 28.0 equiv) was stirred at 110° C. for 1 hour under N₂ atmosphere. The mixture was poured into ice-water (5 mL) and extracted with ethyl acetate (5 mL×2). The combined organic phases were washed with brine (10 mL), dried over anhydrous Na₂SO₄, concentrated, and purified with prep-HPLC [C18, 0.1% formic acid condition] to afford the title compound (60 mg, 85% yield) as a white solid. ¹H NMR (400 MHZ, CHLOROFORM-d) δ=9.25 (d, J=3.0 Hz, 1H), 7.70 (dd, J=6.0, 8.8 Hz, 1H), 7.53 (d, J=2.8 Hz, 1H), 7.29 (s, 1H), 7.25 (s, 1H), 5.40-5.24 (m, 2H), 4.67-4.58 (m, 2H), 4.52-4.37 (m, 3H), 4.25 (br t, J=12.4 Hz, 1H), 3.97-3.68 (m, 1H), 3.53 (d, J=0.8 Hz, 4H), 2.87-2.67 (m, 2H), 2.64-2.41 (m, 8H), 2.38-2.17 (m, 2H), 2.14-2.00 (m, 1H), 1.99-1.75 (m, 2H), 0.93-0.79 (m, 5H), 0.68 (br s, 2H).

Step C. (S)-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-5-ethyl-6-fluoronaphthalen-2-ol and (R)-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-5-ethyl-6-fluoronaphthalen-2-ol: To solution of 1-[1-[[7-[8-ethyl-7-fluoro-3-(methoxymethoxy)-1-naphthyl]-8-fluoro-4-(1-oxa-8-azaspiro[3.5]nonan-8-yl)pyrido[4,3-d]pyrimidin-2-yl]oxymethyl]cyclopropyl]-N,N-dimethylmethanamine (40.0 mg, 1.0 equiv) in DCM (1.0 mL) was added TFA (108 mg, 15 equiv) at 0° C. The reaction mixture was stirred at 25° C. for 1 hour under N₂ atmosphere. The mixture was poured into sat. NaHCO₃ (5 mL) and extracted with ethyl acetate (5 mL×2). The combined organic phases were concentrated and purified with prep-HPLC [C18, 0.1% formic acid condition] to afford the two title compounds:

EXAMPLE 699 (10.6 mg, 27% yield) as white solid. ¹H NMR (400 MHZ, METHANOL-d₄) δ=9.31 (d, J=6.8 Hz, 1H), 7.71 (dd, J=6.0, 8.8 Hz, 1H), 7.34 (d, J=2.8 Hz, 1H), 7.28 (t, J=9.2 Hz, 1H), 7.10 (d, J=2.4 Hz, 1H), 4.82-4.35 (m, 7H), 3.90 (s, 1H), 3.57-3.37 (m, 1H), 3.11-2.93 (m, 2H), 2.86-2.67 (m, 6H), 2.60-2.44 (m, 3H), 2.37-2.27 (m, 1H), 2.27-2.13 (m, 1H), 2.12-1.71 (m, 3H), 0.92 (s, 2H), 0.87-0.80 (m, 3H), 0.77 (br s, 2H); ¹⁹F NMR (376 MHZ, METHANOL-d₄) δ=−121.06 (br s, 1F), −139.88 (br s, 1F); LCMS (ESI, M+1): m/z=590.5.

EXAMPLE 700 (8.8 mg, 16% yield) as white solid. ¹H NMR (400 MHZ, METHANOL-d₄) δ=9.32 (d, J=7.6 Hz, 1H), 7.71 (dd, J=6.0, 9.2 Hz, 1H), 7.34 (d, J=2.8 Hz, 1H), 7.28 (t, J=9.2 Hz, 1H), 7.10 (d, J=2.0 Hz, 1H), 4.84-4.66 (m, 1H), 4.64-4.40 (m, 5H), 3.83 (dd, J=9.6, 13.6 Hz, 1H), 3.58-3.36 (m, 1H), 3.26-3.10 (m, 2H), 2.89 (s, 6H), 2.63-2.44 (m, 3H), 2.43-2.27 (m, 1H), 2.27-2.12 (m, 1H), 2.11-1.89 (m, 2H), 1.88-1.73 (m, 1H), 1.04-0.91 (m, 2H), 0.90-0.74 (m, 5H); ¹⁹F NMR (376 MHZ, METHANOL-d₄) δ=−121.05 (br s, 1F), −140.12 (br s, 1F); LCMS (ESI, M+1): m/z=590.5.

### Example 701

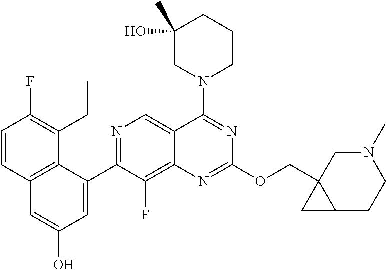 [SMILES: CCc1c(F)ccc2cc(O)cc(-c3ncc4c(N5CCC[C@@](C)(O)C5)nc(OCC56CC5CCN(C)C6)nc4c3F)c12]

### (3R)-1-(7-(8-ethyl-7-fluoro-3-hydroxynaphthalen-1-yl)-8-fluoro-2-((3-methyl-3-azabicyclo[4.1.0]heptan-1-yl)methoxy)pyrido[4,3-d]pyrimidin-4-yl)-3-methylpiperidin-3-ol

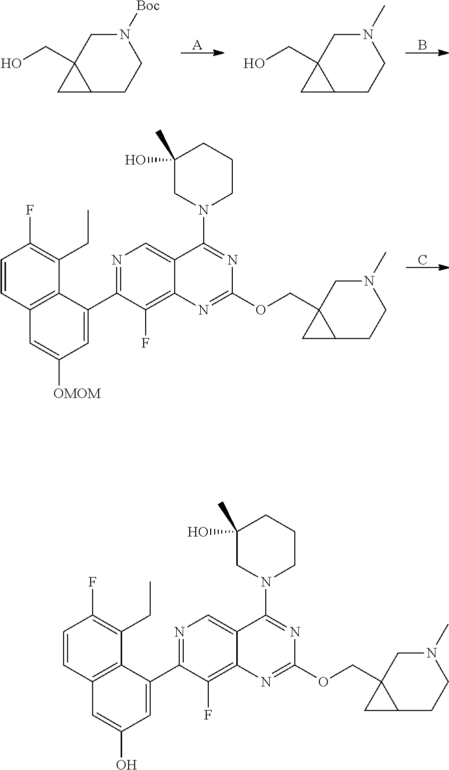

Step A: (3-methyl-3-azabicyclo[4.1.0]heptan-1-yl)methanol: To a solution of tert-butyl 1-(hydroxymethyl)-3-azabicyclo[4.1.0]heptane-3-carboxylate (100 mg, 1.0 equiv) in THF (5.00 mL) was slowly added lithium aluminum hydride (25.1 mg, 1.5 equiv) at 0° C. The mixture was stirred at 60° C. for 4 hours. The reaction mixture was quenched by addition of water (25.0 μL), 15% NaOH solution (25.0 μL) and water (75.0 μL) at 0° C., and then diluted with ethyl acetate (10.0 mL), dried over anhydrous sodium sulfate, filtered and concentrated under reduced pressure to afford the title compound (150 mg, crude) as a colorless oil; MS (ESI, M+1): m/z=142.2.

Step B: (3R)-1-(7-(8-ethyl-7-fluoro-3-(methoxymethoxy)naphthalen-1-yl)-8-fluoro-2-((3-methyl-3-azabicyclo[4.1.0]heptan-1-yl)methoxy)pyrido[4,3-d]pyrimidin-4-yl)-3-methylpiperidin-3-ol: To a solution of (3-methyl-3-azabicyclo[4.1.0]heptan-1-yl)methanol (150 mg, 2.0 equiv) in THF (5.00 mL) was added t-BuOK/THF (1 M, 1.33 mL, 2.5 equiv). The mixture was stirred at 20° C. for 0.5 hour. Then the mixture was added to (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (281 mg, 1.0 equiv) in THF (5.00 mL) The mixture and stirred at 60° C. for 12 hours. The reaction mixture was diluted with water (50.0 mL) and extracted with ethyl acetate (50.0 mL×2). The combined organic layers were washed with brine (50.0 mL), dried over anhydrous sodium sulfate, filtered and concentrated under reduced pressure to give a residue. The residue was purified by prep-TLC (SiO₂, dichloromethane/methyl alcohol 10:1) to afford the title compound (55.0 mg, 16% yield) as a yellow solid; LCMS (ESI, M+1): m/z=634.3.

Step C: (3R)-1-(7-(8-ethyl-7-fluoro-3-hydroxynaphthalen-1-yl)-8-fluoro-2-((3-methyl-3-azabicyclo[4.1.0]heptan-1-yl)methoxy)pyrido[4,3-d]pyrimidin-4-yl)-3-methylpiperidin-3-ol: A solution of (3R)-1-[7-[8-ethyl-7-fluoro-3-(methoxymethoxy)-1-naphthyl]-8-fluoro-2-[(3-methyl-3-azabicyclo[4.1.0]heptan-1-yl)methoxy]pyrido[4,3-d]pyrimidin-4-yl]-3-methyl-piperidin-3-ol (55.0 mg, 1.0 equiv) in HCl/MeOH (2.50 mL) was stirred at 0° C. for 1 hour. The reaction mixture was concentrated and was purified by prep-HPLC (FA condition; column: Phenomenex luna C18 150×25 mm×10 um; mobile phase: [water (FA)-ACN]; B %: 18%-48%, 10 min) and lyophilized to afford the title compound (13.5 mg, 25% yield) as a yellow solid (0.74 formic acid salt). ¹H NMR (400 MHZ, CD₃OD) δ=9.25-9.20 (m, 1H), 8.53 (s, 1H), 7.68 (dd, J=6.0, 8.8 Hz, 1H), 7.35-7.20 (m, 2H), 7.05 (s, 1H), 4.59-4.49 (m, 1H), 4.48-4.21 (m, 3H), 3.68-3.54 (m, 1H), 3.53-3.38 (m, 2H), 3.30-3.23 (m, 1H), 3.09 (br d, J=13.2 Hz, 1H), 2.90-2.76 (m, 1H), 2.62 (s, 3H), 2.54-2.41 (m, 1H), 2.36-2.10 (m, 3H), 2.04-1.93 (m, 1H), 1.91-1.73 (m, 3H), 1.46-1.23 (m, 4H), 1.13-0.98 (m, 1H), 0.86-0.74 (m, 4H); ¹⁹F NMR (376 MHz, CD₃OD) δ=−121.12 (s, 1F), −139.44 (s, 1F). LCMS (ESI, M+1): m/z=590.3.

### Example 702

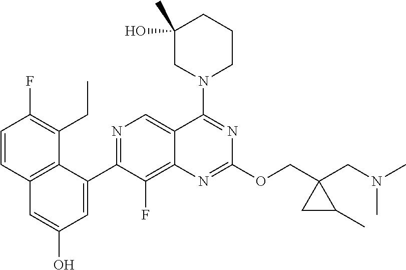 [SMILES: CCc1c(F)ccc2cc(O)cc(-c3ncc4c(N5CCC[C@@](C)(O)C5)nc(OCC5(CN(C)C)CC5C)nc4c3F)c12]

### (3R)-1-(2-((1-((dimethylamino)methyl)-2-methylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

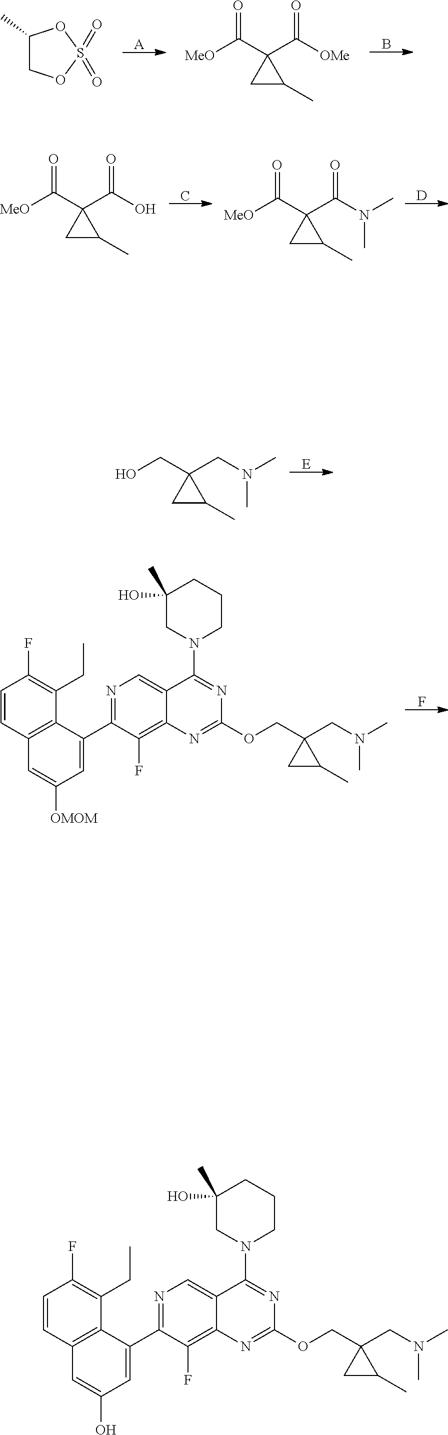

Step A. dimethyl 2-methylcyclopropane-1,1-dicarboxylate: To a solution of dimethyl propanedioate (3.70 g, 1.0 equiv) in DME (40.0 mL) was added NaH (2.30 g, 60% purity, 2.0 equiv) slowly at 0° C. under nitrogen atmosphere. The mixture was stirred at 0° C. for 0.5 hour. Then (S)-4-methyl-1,3,2-dioxathiolane 2,2-dioxide (3.90 g, 1.0 equiv) in DME (40.0 mL) was added dropwise at 0° C. and the resulting mixture warmed to 25° C. and stirred at 25° C. for 2 hours. The reaction mixture was quenched with ammonium chloride solution (80.0 mL) dropwise at 0° C., and extracted with ethyl acetate (40.0 mL×3). The combined organic layer was dried over Na₂SO₄, concentrated and purified with column chromatography [SiO₂, petroleum ether/ethyl acetate 1:0 to 10:1] to afford the title compound (3.00 g, 62% yield) as a colorless oil. ¹H NMR (400 MHZ, CHLOROFORM-d) δ=3.76 (s, 3H), 3.71 (s, 3H), 1.98-1.88 (m, 1H), 1.42 (dd, J=4.4, 9.0 Hz, 1H), 1.34 (dd, J=4.4, 7.6 Hz, 1H), 1.09 (d, J=6.4 Hz, 3H).

Step B. 1-(methoxycarbonyl)-2-methylcyclopropane-1-carboxylic acid: To a solution of dimethyl 2-methylcyclopropane-1,1-dicarboxylate (3.00 g, 1.0 equiv) in MeOH (30.0 mL) was added NaOH (1.7 M, 1.20 equiv) and THF (125 mg). The mixture was stirred at 30° C. for 12 hours. The mixture was concentrated and purified by column chromatography [SiO₂, ethyl acetate/MeOH 1:0 to 10/1] to afford the tittle compound (2.50 g, 91% yield) as a colorless oil; ¹H NMR (400 MHZ, DMSO-d₆) δ=3.59 (s, 3H), 1.53-1.47 (m, 1H), 1.12-1.05 (m, 2H), 0.94 (d, J=6.4 Hz, 3H).

Step C. methyl 1-(dimethylcarbamoyl)-2-methylcyclopropane-1-carboxylate: To a solution of 1-(methoxycarbonyl)-2-methylcyclopropane-1-carboxylic acid (2.50 g. 1.0 equiv) in DMF (40.0 mL) was added HATU (18.0 g, 3.0 equiv) and DIPEA (20.4 g, 10 equiv) at 20° C. After addition, the mixture was stirred at this temperature for 0.5 hour, and then N-methylmethanamine (1.80 g, 40% purity, 1.0 equiv) was added at 20° C. The resulting mixture was stirred at 20° C. for 11.5 hours. The reaction mixture was diluted with water (80.0 mL) and extracted with ethyl acetate (40.0 mL×3). The combined organic layer was dried over Na₂SO₄, concentrated and purified with column chromatography [SiO₂, petroleum ether/ethyl acetate 10:1 to 3:1] to afford the title compound (1.00 g, 34% yield) as a brown solid. ¹H NMR (400 MHZ, CHLOROFORM-d) δ=3.74 (s, 3H), 3.01 (s, 3H), 2.97 (s, 3H), 1.91-1.81 (m, 1H), 1.43 (dd, J=4.4, 7.2 Hz, 1H), 1.33 (dd, J=4.4, 9.2 Hz, 1H), 1.25 (d, J=6.4 Hz, 3H)

Step D. (1-((dimethylamino)methyl)-2-methylcyclopropyl)methanol: To a solution of methyl 1-(dimethylcarbamoyl)-2-methylcyclopropane-1-carboxylate (1.00 g, 1.0 equiv) in THF (10.0 mL) was added LiAlH₄ (819 mg, 4.0 equiv) at 0° C. The mixture was stirred at 20° C. for 1 hour. The reaction mixture was quenched by addition of water (0.8 mL), 15% NaOH solution (0.8 mL) and water (2.4 mL) at 0° C., and then diluted with ethyl acetate. The mixture was filtered, the filtrate was concentrated and purified by reversed phase flash chromatography [column: Waters Xbridge 150×25 mm×5 um; mobile phase: [water (NH₄HCO₃)-ACN]; B %: 1%-30%, 10 minutes] to afford the title compound (240 mg 31% yield) as a colorless oil.

Step E. (3R)-1-(2-((1-((dimethylamino)methyl)-2-methylcyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of (1-((dimethylamino)methyl)-2-methylcyclopropyl)methanol (50.0 mg, 1.0 equiv) and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (184 mg, 1.0 equiv) in dioxane (2.00 mL) was added Cs₂CO₃ (341 mg, 3.0 equiv) at −78° C. The mixture was stirred at 25° C. for 2 hours. The reaction mixture was diluted with water (30.0 mL) and extracted with ethyl acetate (15.0 mL×3). The combined organic layer was dried over Na₂SO₄, concentrated and purified with prep-TLC (SiO₂, dichloromethane/methyl alcohol 10:1) to afford the title compound (6.00 mg, 3% yield) as a white solid.

Step F. (3R)-1-(2-((1-((dimethylamino)methyl)-2-methylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: A solution of (3R)-1-(2-((1-((dimethylamino)methyl)-2-methylcyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (6.00 mg, 1.0 equiv) in HCl/MeOH (0.50 mL) was stirred at 20° C. for 1 hour. The mixture was filtered, concentrated and purified by reversed phase flash chromatography [column: Phenomenex luna C18 150×25 mm×10 um; mobile phase: [water (FA)-ACN]; B %: 18%-48%, 10 minutes] and lyophilized to afford the title compound (2.00 mg, 33% yield) as a white solid (0.69 formic acid salt); ¹H NMR (400 MHZ, METHANOL-d₄) δ=9.24 (d, J=2.0 Hz, 1H), 8.54 (s, 1H), 7.68 (dd, J=6.0, 9.0 Hz, 1H), 7.31 (d, J=2.8 Hz, 1H), 7.26 (t, J=9.2 Hz, 1H), 7.06 (t, J=2.8 Hz, 1H), 4.69 (d, J=12.0 Hz, 1H), 4.60-4.55 (m, 1H), 4.47 (d, J=12.0 Hz, 1H), 4.31 (br t, J=13.2 Hz, 1H), 3.69-3.58 (m, 1H), 3.52-3.40 (m, 1H), 3.05-2.79 (m, 2H), 2.69 (br s, 6H), 2.54-2.39 (m, 1H), 2.26-2.10 (m, 2H), 1.92-1.73 (m, 3H), 1.33-1.23 (m, 6H), 1.14-1.04 (m, 1H), 0.90-0.76 (m, 4H), 0.61 (t, J=5.2 Hz, 1H); LCMS (ESI, M+1): m/z=592.3.

### Example 703

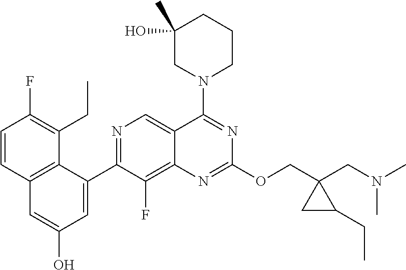 [SMILES: CCc1c(F)ccc2cc(O)cc(-c3ncc4c(N5CCC[C@@](C)(O)C5)nc(OCC5(CN(C)C)CC5CC)nc4c3F)c12]

### (3R)-1-(2-((1-((dimethylamino)methyl)-2-ethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

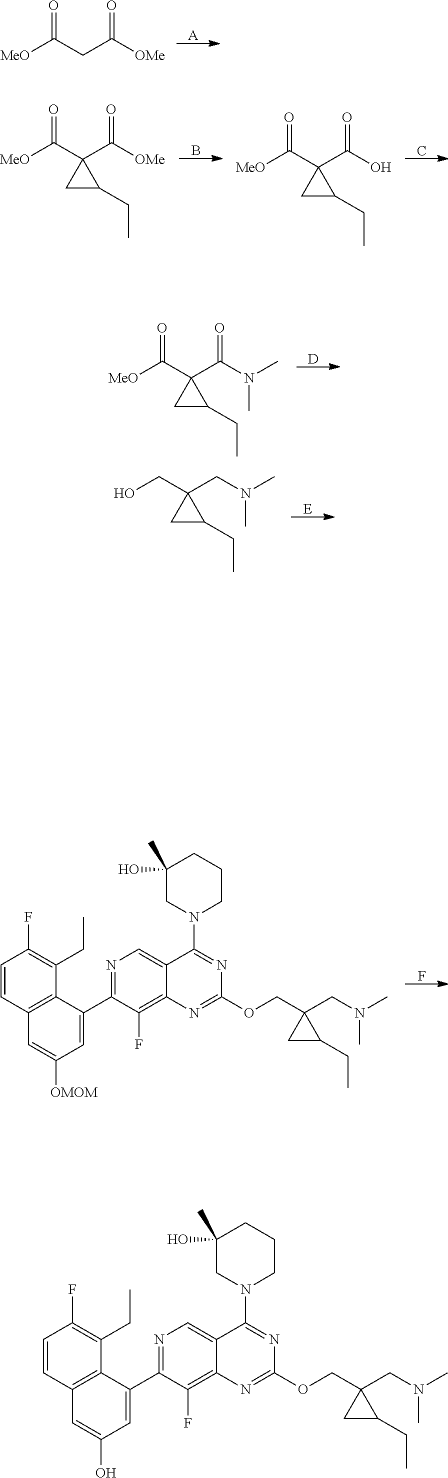

Step A. dimethyl 2-ethylcyclopropane-1,1-dicarboxylate: To a mixture of dimethyl propanedioate (1.00 g, 1.0 equiv) and 1,2-dibromobutane (2.1 g, 1.3 equiv) in DMF (20 mL) was added K₂CO₃ (3.10 g, 3.0 equiv) and TBAB (2.40 g, 1.0 equiv) at 25° C. under N₂. The mixture was stirred at 25° C. for 12 hours. The reaction was poured into ice-water (15 mL) and stirred for 1 min. The aqueous phase was extracted with ethyl acetate (20 mL×3). The combined organic phases were washed with brine (25 mL), dried with anhydrous Na₂SO₄, filtered and concentrated in vacuum. The residue was purified by silica gel chromatography (column height: 250 mm, diameter: 100 mm, 100-200 mesh silica gel, Petroleum ether/Ethyl acetate=20/1, 10/1) to afford the tittle compound (1.20 g, 85% yield) as colorless oil. ¹H NMR (400 MHZ, CHLOROFORM-d) δ=3.76 (s, 3H), 3.72 (s, 3H), 1.88 (quin, J=7.6 Hz, 1H), 1.50-1.35 (m, 3H), 1.30-1.20 (m, 1H), 1.03-0.98 (m, 3H).

Steps B-F. (3R)-1-(2-((1-((dimethylamino)methyl)-2-ethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: The title compound was synthesized from dimethyl 2-ethylcyclopropane-1,1-dicarboxylate according to the 5-step procedure described in Steps B-F for example 702 to produce the desired compound as a white solid (0.38 formic acid salt). ¹H NMR (400 MHZ, METHANOL-d₄) δ=9.23 (dd, J=2.0, 4.0 Hz, 1H), 8.54 (s, 1H), 7.68 (dd, J=5.6, 8.8 Hz, 1H), 7.31 (d, J=2.8 Hz, 1H), 7.25 (t, J=9.2 Hz, 1H), 7.06 (s, 1H), 4.66 (d, J=12.0 Hz, 1H), 4.60-4.45 (m, 2H), 4.29 (br t, J=12.0 Hz, 1H), 3.70-3.56 (m, 1H), 3.52-3.39 (m, 1H), 2.94-2.77 (m, 2H), 2.64 (br s, 6H), 2.53-2.40 (m, 1H), 2.28-2.08 (m, 2H), 1.91-1.74 (m, 3H), 1.66 (td, J=7.2, 14.0 Hz, 1H), 1.44 (td, J=7.2, 14.4 Hz, 1H), 1.29 (d, J=9.6 Hz, 3H), 1.12-1.03 (m, 3H), 1.01-0.93 (m, 1H), 0.87-0.76 (m, 4H), 0.59 (br t, J=5.2 Hz, 1H); LCMS (ESI, M+1): m/z=606.5.

### Example 704

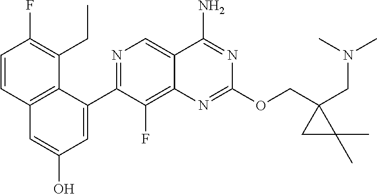 [SMILES: CCc1c(F)ccc2cc(O)cc(-c3ncc4c(N)nc(OCC5(CN(C)C)CC5(C)C)nc4c3F)c12]

### (3R)-1-(2-((1-((dimethylamino)methyl)-2,2-dimethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

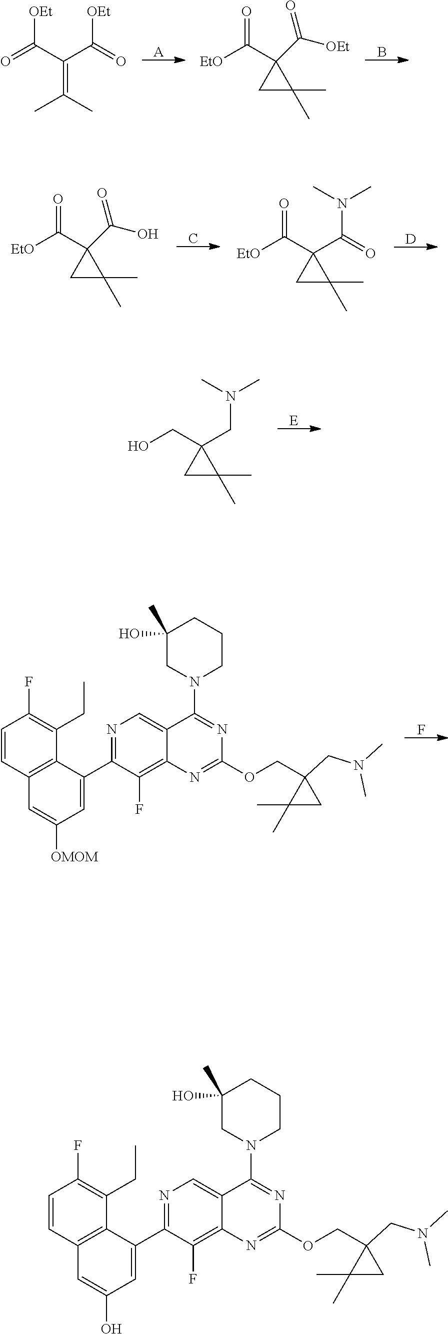

Step A. diethyl 2,2-dimethylcyclopropane-1,1-dicarboxylate: To a solution of trimethylsulfoxonium iodide (4.95 g, 1.5 equiv) in DMSO (15 mL) was added NaH (899 mg, 60% purity, 1.5 equiv). The mixture was stirred at 0° C. for 0.5 hour. Diethyl 2-isopropylidenepropanedioate (3 g, 1.0 equiv) was added. The mixture was stirred at 0° C. for 12 hours. The reaction mixture was quenched by addition NH₄Cl (sat. 10 mL) at 0° C., and then diluted with water (20 mL) and extracted with EtOAc (20 mL×3). The combined organic layers were washed with brine (50 mL×2), dried over Na₂SO₄, filtered and concentrated under reduced pressure to give a residue. The residue was purified by flash silica gel chromatography [ISCO®; 40 g SepaFlash® Silica Flash Column, Eluent of 0˜25% Ethyl acetate/Petroleum ethergradient @ 50 mL/min] to afford the tittle compound (2.3 g, 72% yield) as yellow oil; ¹H NMR (400 MHZ, CHLOROFORM-d) δ=4.13 (ttd, J=3.6, 7.2, 10.8 Hz, 4H), 1.35 (s, 2H), 1.21 (t, J=7.2 Hz, 6H), 1.18 (s, 6H).

Steps B-F. (3R)-1-(2-((1-((dimethylamino)methyl)-2,2-dimethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: The title compound was synthesized from diethyl 2,2-dimethylcyclopropane-1,1-dicarboxylate according to the 5-step procedure described in Steps B-F for example 702 to produce the desired compound as a white solid (0.48 formic acid salt). ¹H NMR (400 MHZ, METHANOL-d₄) δ=9.24 (dd, J=3.6, 8.0 Hz, 1H), 8.56 (s, 1H), 7.70 (dd, J=6.0, 9.2 Hz, 1H), 7.33 (d, J=2.8 Hz, 1H), 7.27 (t, J=9.2 Hz, 1H), 7.08 (s, 1H), 4.83-4.78 (m, 1H), 4.60-4.54 (m, 1H), 4.47-4.38 (m, 1H), 4.31 (br t, J=12.4 Hz, 1H), 3.74-3.58 (m, 1H), 3.56-3.42 (m, 1H), 3.29-3.13 (m, 1H), 2.87-2.73 (m, 1H), 2.60 (br s, 5H), 2.51-2.42 (m, 1H), 2.34-2.11 (m, 2H), 1.96-1.75 (m, 3H), 1.40-1.28 (m, 6H), 1.27-1.15 (m, 3H), 0.93-0.77 (m, 4H), 0.66 (br d, J=4.8 Hz, 1H); ¹⁹F NMR (377 MHz, METHANOL-d₄) δ=−121.11 (br s, 1F), −139.72 (br s, 1F); LCMS (ESI, M+1): m/z=606.4.

### Example 705

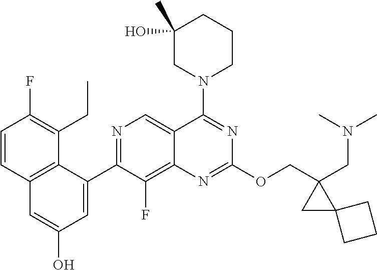 [SMILES: CCc1c(F)ccc2cc(O)cc(-c3ncc4c(N5CCC[C@@](C)(O)C5)nc(OCC5(CN(C)C)CC56CCC6)nc4c3F)c12]

### (3R)-1-[2-[[2-[(dimethylamino)methyl]spiro[2.3]hexan-2-yl]methoxy]-7-(8-ethyl-7-fluoro-3-hydroxy-1-naphthyl)-8-fluoro-pyrido[4,3-d]pyrimidin-4-yl]-3-methyl-piperidin-3-ol

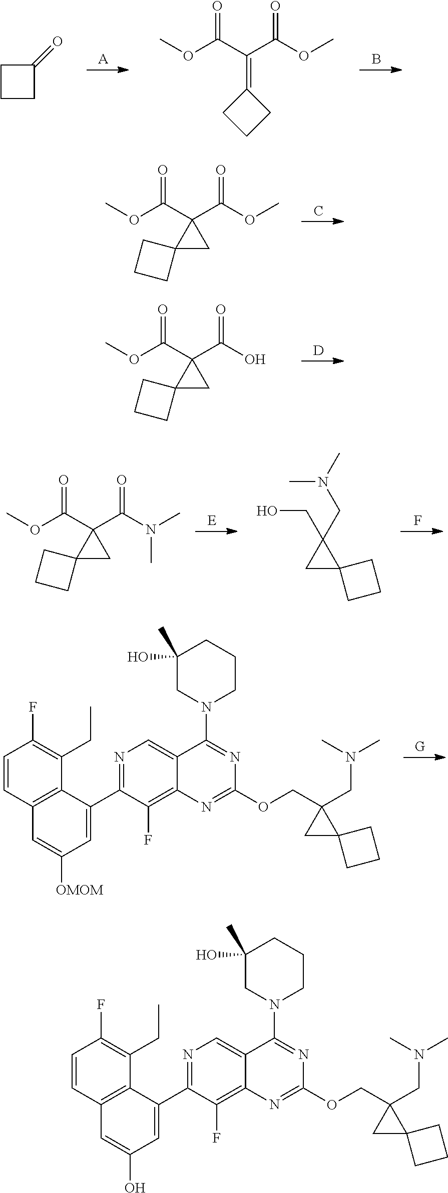

Step A. dimethyl 2-cyclobutylidenepropanedioate: To a solution of cyclobutanone (10 g, 1.0 equiv) and dimethyl propanedioate (18.9 g, 1.0 equiv) in toluene (100 mL) was added TiCl₄ (29.8 g, 1.1 equiv) and pyridine (22.6 g, 2.0 equiv) at 25° C. The mixture was stirred at 25° C. for 12 hours and concentrated. The residue was purified by column chromatography (SiO₂, Petroleum ether/Ethyl acetate 20:1 to 10:1) to afford the title compound (14.3 g, 54% yield) as a yellow oil.

Step B. dimethyl spiro[2.3]hexane-2,2-dicarboxylate: NaH (3.3 g, 60% purity, 1.5 equiv) was suspended in anhydrous DMSO (100 mL) in a flame-dried round-bottom flask under N₂. Trimethylsulfoxonium iodide (17.9 g, 1.5 equiv) was added and the solution stirred at 0° C. for 1 hour. Then dimethyl 2-cyclobutylidenepropanedioate (10.0 g, 1.0 equiv) in anhydrous DMSO (20 mL) was added. The mixture was stirred at 25° C. for 11 hours. The residue was purified by column chromatography (SiO₂, Petroleum ether/Ethyl acetate 20:1 to 10:1) to afford the title compound (5.2 g, 48% yield) as a yellow oil. ¹H NMR (400 MHZ, CHLOROFORM-d) δ1.64 (s, 2H) 1.86-1.97 (m, 1H) 2.02-2.10 (m, 3H) 2.28-2.40 (m, 2H) 3.70 (s, 6H).

Step C. 2-methoxycarbonylspiro[2.3]hexane-2-carboxylic acid: To a solution of dimethyl spiro[2.3]hexane-2,2-dicarboxylate (1.2 g, 1.0 equiv) in EtOH (20 mL) was added a solution of NaOH (1.0 M, 6.7 mL, 1.1 equiv) drop-wise at 0° C. under N₂. The reaction mixture was warmed to 25° C. and stirred at 25° C. for 2 hours. The residue was poured into ice-water (20 mL) and extracted with DCM (20 mL×2). Then HCl (1 M, 5 mL) was added and the resulting was stirred for 2 mins. The aqueous phase was extracted with DCM (20 mL×3). The combined organic layers were washed with brine (50 mL), dried with anhydrous Na₂SO₄, filtered and concentrated under reduced pressure to afford the title compound (840 mg, crude) as a white solid.

Step D. methyl 2-(dimethylcarbamoyl)spiro[2.3]hexane-2-carboxylate: To a solution of 2-methoxycarbonylspiro[2.3]hexane-2-carboxylic acid (840 mg, 1.0 equiv) and N-methylmethanamine (744 mg, 2.0 equiv, HCl) in DCM (10 mL) were added HATU (3.5 g, 2.0 equiv) and DIPEA (3.0 g, 5.0 equiv) at 25° C. The mixture was stirred at 25° C. for 12 hours. The residue was purified by column chromatography (SiO₂, petroleum ether/ethyl acetate 10:1 to 1:4) to afford the title compound (765 mg, 79% yield) as a colorless liquid; ¹H NMR (400 MHZ, CHLOROFORM-d) δ 1.54-1.63 (m, 2H) 1.89-2.01 (m, 2H) 2.07-2.18 (m, 1H) 2.21-2.31 (m, 2H) 2.41-2.52 (m, 1H) 2.99 (s, 3H) 3.05 (s, 3H) 3.69 (s, 3H).

Step E. [2-[(dimethylamino)methyl]spiro[2.3]hexan-2-yl]methanol: To a solution of methyl 2-(dimethylcarbamoyl)spiro[2.3]hexane-2-carboxylate (765 mg, 1.0 equiv) in THF (10 mL) was added LiAlH₄ (687 mg, 5.0 equiv) at 0° C. The mixture was stirred at 25° C. for 2 hours. The residue was poured into Na₂SO₄ (sat., 2 mL) at 0° C. and stirred for 5 minutes. The reaction mixture was filtered and the filterate was concentrated to afford the title compound (552 mg, crude) as a colorless liquid.

Step F. (3R)-1-[2-[[2-[(dimethylamino)methyl]spiro[2.3]hexan-2-yl]methoxy]-7-[8-ethyl-7-fluoro-3-(methoxymethoxy)-1-naphthyl]-8-fluoro-pyrido[4,3-d]pyrimidin-4-yl]-3-methyl-piperidin-3-ol: To a solution of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (200 mg, 1.0 equiv) and [2-[(dimethylamino)methyl]spiro[2.3]hexan-2-yl]methanol (64.0 mg, 1.0 equiv) in THF (1.5 mL) was added NaHMDS (1 M, 567 μL, 1.5 equiv) at 0° C. The mixture was stirred at 25° C. for 12 hours. The residue was concentrated and purified by prep-HPLC [C18, 0.1% formic acid condition] to afford the title compound (81 mg, 32% yield) as a yellow solid.

Step G. (3R)-1-[2-[[2-[(dimethylamino)methyl]spiro[2.3]hexan-2-yl]methoxy]-7-(8-ethyl-7-fluoro-3-hydroxy-1-naphthyl)-8-fluoro-pyrido[4,3-d]pyrimidin-4-yl]-3-methyl-piperidin-3-ol: To a solution of (3R)-1-[2-[[2-[(dimethylamino)methyl]spiro[2.3]hexan-2-yl]methoxy]-7-[8-ethyl-7-fluoro-3-(methoxymethoxy)-1-naphthyl]-8-fluoro-pyrido[4,3-d]pyrimidin-4-yl]-3-methyl-piperidin-3-ol (28 mg, 1.0 equiv) in dioxane (1 mL) was added HCl/dioxane (4 M, 0.5 mL, 47.3 equiv) at 0° C. . . . The mixture was stirred at 25° C. for 1 hour. The mixture was concentrated and purified by prep-HPLC [C18, 0.1% formic acid condition] and lyophilized to afford the title compound (2.7 mg, 9.3% yield) as a yellow solid (0.72 formic acid salt). ¹H NMR (400 MHz, METHANOL-d₄) δ=0.83 (br dd, J=7.2, 3.6 Hz, 3H), 0.95 (br d, J=3.6 Hz, 1H), 1.07 (br d, J=4.8 Hz, 1H), 1.20 (t, J=7.2 Hz, 2H), 1.31 (br d, J=8.4 Hz, 3H), 1.74-1.94 (m, 3H), 1.98-2.13 (m, 3H), 2.13-2.33 (m, 4H), 2.37-2.60 (m, 4H), 3.41-3.52 (m, 1H), 3.53-3.70 (m, 3H), 4.19 (br dd, J=11.2, 6.8 Hz, 1H), 4.28-4.39 (m, 1H), 4.60 (dt, J=7.69, 3.78 Hz, 3H), 7.08 (br s, 1H), 7.28 (br t, J=9.2 Hz, 1H), 7.33 (br d, J=2.0 Hz, 1H), 7.70 (br dd, J=8.8, 5.6 Hz, 1H), 8.47-8.62 (m, 1H), 9.21-9.32 (m, 1H); LCMS (ESI, M+1): m/z=618.4.

### Example 706

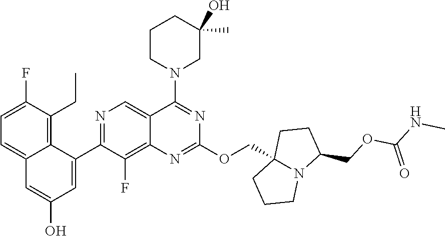 [SMILES: CCc1c(F)ccc2cc(O)cc(-c3ncc4c(N5CCC[C@@](C)(O)C5)nc(OC[C@@]56CCCN5[C@H](COC(=O)NC)CC6)nc4c3F)c12]

### ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl methylcarbamate

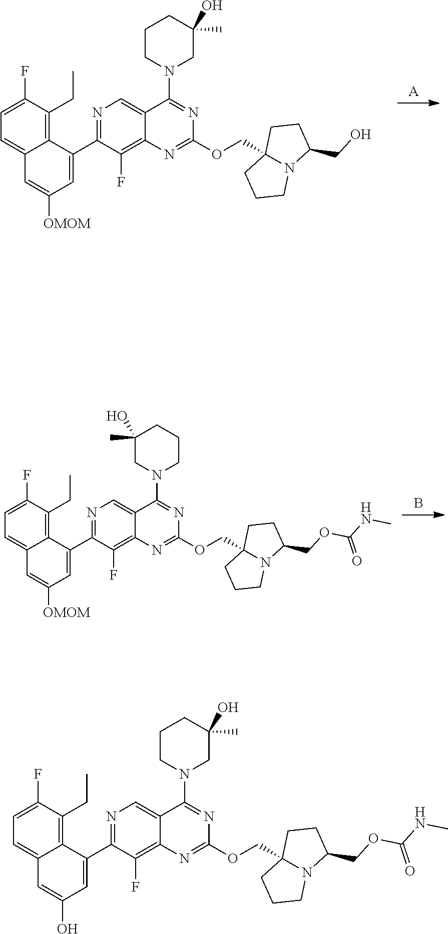

Step A. ((3S,7aS)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl methylcarbamate: To a solution of (R)-1-(7-(8-ethyl-7-fluoro-3-(methoxymethoxy)naphthalen-1-yl)-8-fluoro-2-(((3S,7aS)-3-(hydroxymethyl)hexahydro-1H-pyrrolizin-7a-yl)methoxy)pyrido[4,3-d]pyrimidin-4-yl)-3-methylpiperidin-3-ol (300 mg, 1.0 equiv) in tetrahydrofuran (2 mL) was added NaH (36.1 mg, 60% purity, 2.0 equiv) at 0° C., the mixture was stirred at 0° C. for 0.5 hour. Then N-methylcarbamoyl chloride (84.5 mg, 2.0 equiv) in tetrahydrofuran (2 mL) was added and the mixture was stirred at 0° C. for 2 hours. The reaction mixture was quenched with water (5 mL) and was extracted with ethyl acetate (2×5 mL). The combined organic layers were dried over anhydrous sodium sulfate, concentrated and purified by column chromatography (SiO₂, dichloromethane:methanol 5:1) to afford the title compound (40.0 g, 11% yield) as a colorless oil; LCMS (ESI, M+1): m/z=721.3.

Step B. ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl methylcarbamate: To a solution of ((3S,7aS)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl methylcarbamate (40.0 mg, 1.0 equiv) in acetonitrile (0.5 mL) was added HCl·MeOH (4 M, 36 equiv). The mixture was stirred at 0° C. for 0.5 hour. The reaction mixture was concentrated and diluted with acetonitrile (3 mL) and the pH of the mixture was adjusted to ˜ 7 using DIEA. The mixture was purified by prep-HPLC [column: Waters Xbridge 150×25 mm×5 μm; A: water (NH₄HCO₃); B: ACN, B %: 43%-73%, over 9 min] and lyophilized to afford the title compound (6.61 mg, 17% yield) as a white solid; SFC: Chiralcel OJ-3 50×4.6 mm I.D., 3 μm, MeOH (0.05% DEA) in CO₂ from 5% to 40% Flow rate: 3 mL/min, detector: 220 nm, t_{R1}=1.410 min; ¹H NMR (400 MHZ, CHLOROFORM-d) δ=9.12 (d, J=13.2 Hz, 1H), 7.58 (dd, J=5.6, 8.8 Hz, 1H), 7.24-7.16 (m, 2H), 7.06 (dd, J=2.4, 6.4 Hz, 1H), 4.48-4.16 (m, 4H), 4.12-3.96 (m, 2H), 3.68-3.52 (m, 1H), 3.48-3.28 (m, 2H), 3.12-2.92 (m, 3H), 2.24-2.12 (m, 2H), 1.92-1.84 (m, 3H), 1.76-1.64 (m, 4H), 1.48-1.24 (m, 13H), 0.85 (td, J=7.2, 10.8 Hz, 3H); LCMS (ESI, M+1): m/z=677.5.

### Example 707

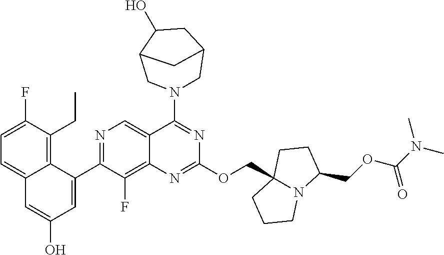 [SMILES: CCc1c(F)ccc2cc(O)cc(-c3ncc4c(N5CC6CC(O)C(C6)C5)nc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-(6-hydroxy-3-azabicyclo[3.2.1]octan-3-yl)pyrido[4,3-d]pyrimidin-2-yl)oxy)methyl)hexahydro-1H-pyrrolizin-3-yl)methyl dimethylcarbamate

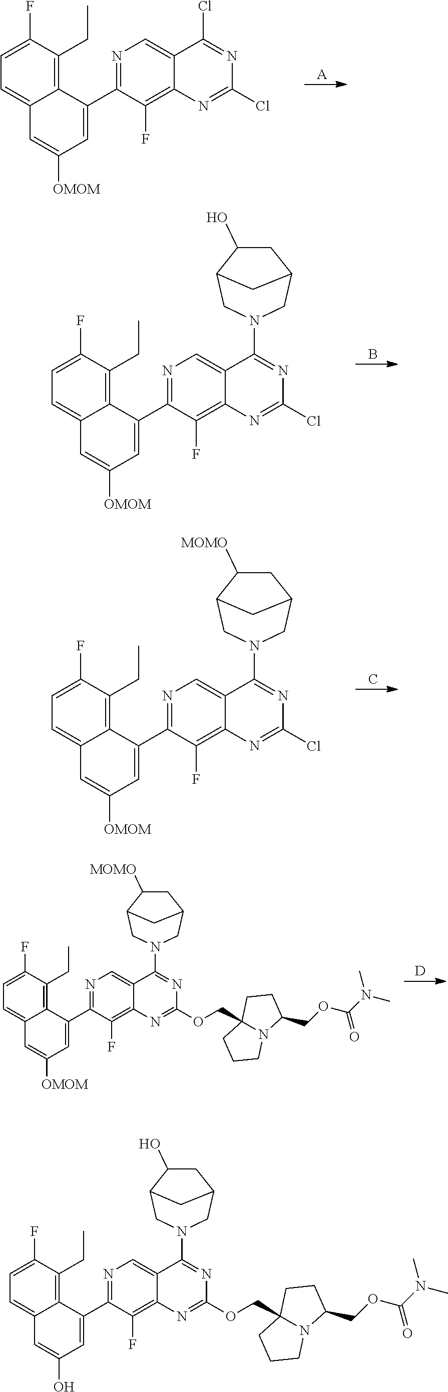

Step A. 3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of 3-azabicyclo[3.2.1]octan-6-ol (1.27 g, 1.5 equiv) and 2,4-dichloro-7-[8-ethyl-7-fluoro-3-(methoxymethoxy)-1-naphthyl]-8-fluoro-pyrido[4,3-d]pyrimidine (3.00 g, 1.0 equiv) in DCM (40 mL) was added DIPEA (2.58 g, 3.0 equiv). The mixture was stirred at 40° C. for 1.5 hr. The reaction mixture was diluted with water (5 mL) and extracted with DCM (2.0 mL×3). The combined organic layers were dried over anhydrous sodium sulfate, concentrated and purified by column chromatography (SiO₂, Petroleum ether/Ethyl acetate=20/1 to 1/1) to afford the title compound (1.90 g, 50% yield) as a white solid; LCMS (ESI, M+1): m/z=541.2.

Step B. 2-chloro-7-(8-ethyl-7-fluoro-3-(methoxymethoxy)naphthalen-1-yl)-8-fluoro-4-(6-(methoxymethoxy)-3-azabicyclo[3.2.1]octan-3-yl)pyrido[4,3-d]pyrimidine: To a solution of 3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (700 mg, 1 equiv) in DCM (10 mL) was added DIPEA (1.00 g, 6.0 equiv) and bromo(methoxy) methane (912 mg, 5.0 eq). The mixture was stirred at 25° C. for 12 hrs. The reaction mixture was filtered. The filtrate was diluted with water (5.0 mL) and extracted with ethyl acetate (5.0 mL×3). The combined organic layers were washed with brine (5.0 mL×3), dried over anhydrous sodium sulfate, filtered, concentrated and purified by reversed-phase HPLC (0.1% FA condition) to afford the title compound (800 mg, 68% yield) as a white solid; LCMS (ESI, M+1): m/z=585.3.

Step C. ((3S,7aR)-7a-(((7-(8-ethyl-7-fluoro-3-(methoxymethoxy)naphthalen-1-yl)-8-fluoro-4-(6-(methoxymethoxy)-3-azabicyclo[3.2.1]octan-3-yl)pyrido[4,3-d]pyrimidin-2-yl)oxy)methyl)hexahydro-1H-pyrrolizin-3-yl)methyl dimethylcarbamate: To a solution of 2-chloro-7-(8-ethyl-7-fluoro-3-(methoxymethoxy)naphthalen-1-yl)-8-fluoro-4-(6-(methoxymethoxy)-3-azabicyclo[3.2.1]octan-3-yl)pyrido[4,3-d]pyrimidine (200 mg, 1.0 equiv) in THF (4 mL) was added t-BuONa (98.6 mg, 3.0 equiv) and ((3S,7aR)-7a-(hydroxymethyl)hexahydro-1H-pyrrolizin-3-yl)methyl dimethylcarbamate (91.1 mg, 1.1 equiv). The mixture was stirred at 0° C. for 0.5 hrs. The reaction mixture was filtered. The filtrate was diluted with water (5.0 mL) and extracted with ethyl acetate (5.0 mL×3). The combined organic layers were washed with brine (5.0 mL×3), dried over anhydrous sodium sulfate, filtered, concentrated and purified by reversed-phase HPLC (0.1% FA condition) to afford the title compound (90.0 mg, 30% yield) was as a yellow oil; LCMS (ESI, M+1): m/z=791.5.

Step D. ((3S,7aR)-7a-(((7-(8-ethyl-7-fluoro-3-hydroxynaphthalen-1-yl)-8-fluoro-4-(6-hydroxy-3-azabicyclo[3.2.1]octan-3-yl)pyrido[4,3-d]pyrimidin-2-yl)oxy)methyl)hexahydro-1H-pyrrolizin-3-yl)methyl dimethylcarbamate: To a solution of ((3S,7aR)-7a-(((7-(8-ethyl-7-fluoro-3-(methoxymethoxy)naphthalen-1-yl)-8-fluoro-4-(6-(methoxymethoxy)-3-azabicyclo[3.2.1]octan-3-yl)pyrido[4,3-d]pyrimidin-2-yl)oxy)methyl)hexahydro-1H-pyrrolizin-3-yl)methyl dimethylcarbamate (90.0 mg, 1.0 equiv) in ACN (0.4 mL) was added HCl/dioxane (4 M, 0.8 mL, 28 equiv). The mixture was stirred at 0° C. for 0.5 hr. The mixture was poured into iced NaHCO₃ (2.0 mL) and filtered. The filtrate was diluted with water (5.0 mL) and extracted with ethyl acetate (5.0 mL×3). The combined organic layers were washed with brine (5.0 mL×3), dried over anhydrous sodium sulfate, concentrated and purified by prep-HPLC (column: YMC Triart C18 150×25 mm×5 um; A: water [(FA)]; B: ACN, B %: 19%-49%, over 10 min) to afford the title compound (30.0 mg. 35% yield) as a yellow solid (0.47 formic acid salt); SFC: Chiralpak AD-3 50×4.6 mm I.D, 3 μm, ACN (0.05% DEA) in CO₂ from 5% to 40% Flow rate: 3 mL/min, detector: 220 nm, t_{R1}=0.745, t_{R2}=0.998, t_{R3}=1.165, t_{R4}=1.727; ¹H NMR (400 MHZ, METHANOL-d4) δ=9.08 (s, 1H), 7.66 (dd, J=6.0, 8.8 Hz, 1H), 7.32-7.29 (m, 1H), 7.26-7.19 (m, 1H), 7.10-7.04 (m, 1H), 5.05-5.0 (m, 1H), 4.85-4.74 (m, 1H), 4.56-4.47 (m, 2H), 4.47-4.10 (m, 3H), 3.85-3.32 (m, 3H), 3.29-3.23 (m, 1H), 3.05 (br s, 6H), 2.59-2.36 (m, 3H), 2.15-1.34 (m, 14H), 0.84 (br t, J=7.2 Hz, 3H); LCMS (ESI, M+1): m/z=703.2.

### Example 708

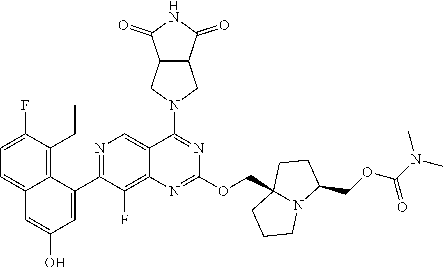 [SMILES: CCc1c(F)ccc2cc(O)cc(-c3ncc4c(N5CC6C(=O)NC(=O)C6C5)nc(OC[C@]56CCCN5[C@H](COC(=O)N(C)C)CC6)nc4c3F)c12]

### ((3S,7aR)-7a-(((4-(4,6-dioxohexahydropyrrolo[3,4-c]pyrrol-2(1H)-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate

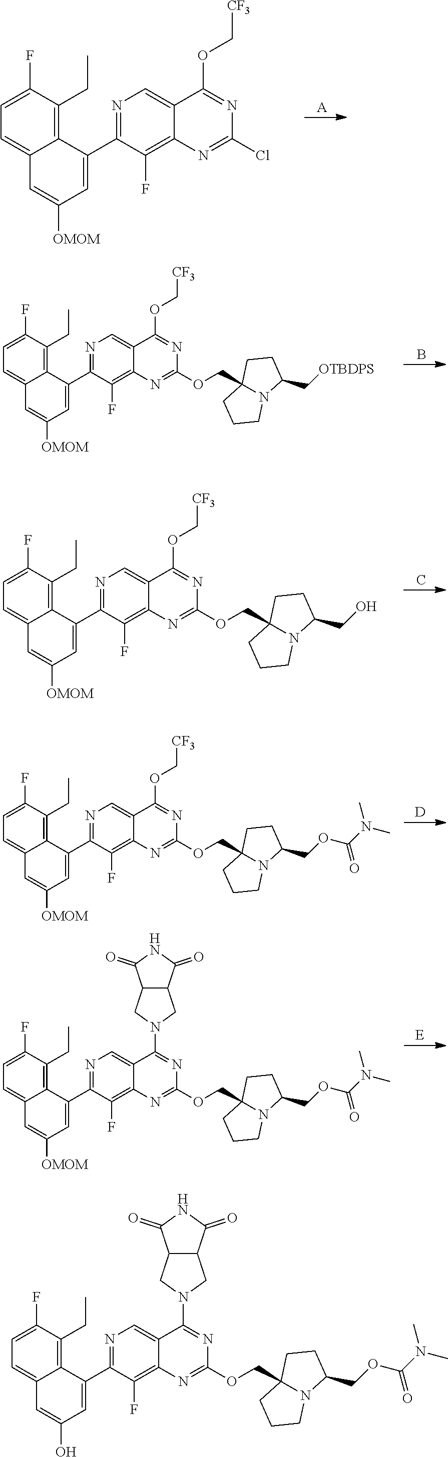

Step A. 2-(((3S,7aR)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoro-4-(2,2,2-trifluoroethoxy)pyrido[4,3-d]pyrimidine: To a mixture of 2-chloro-7-(8-ethyl-7-fluoro-3-(methoxymethoxy)naphthalen-1-yl)-8-fluoro-4-(2,2,2-trifluoroethoxy)pyrido[4,3-d]pyrimidine (10.0 g, 1.0 equiv), ((3S,7aR)-3-(((tert-butyldiphenylsilyl)oxy)methyl)hexahydro-1H-pyrrolizin-7a-yl)methanol (8.77 g, 1.1 equiv) in dioxane (10 mL) was added Na₂CO₃ (6.19 g, 3.0 equiv). The mixture was stirred at 40° C. for 14 hours. The mixture was diluted with water (100 mL) and extracted with ethyl acetate (200 mL). The organic layer was dried over Na₂SO₄ and concentrated to give a residue. The residue was purified by column chromatography [SiO₂, Petroleum ether/Ethyl acetate 5:1 to 2:1] to afford the title compound (7.50 g, 37% yield) as a yellow oil; LCMS (ESI, M+1): m/z=887.5.

Step B. ((3S,7aR)-7a-(((7-(8-ethyl-7-fluoro-3-(methoxymethoxy)naphthalen-1-yl)-8-fluoro-4-(2,2,2-trifluoroethoxy)pyrido[4,3-d]pyrimidin-2-yl)oxy)methyl)hexahydro-1H-pyrrolizin-3-yl)methanol: To a solution of 2-(((3S,7aR)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoro-4-(2,2,2-trifluoroethoxy)pyrido[4,3-d]pyrimidine (6.50 g, 1.0 equiv) in THF (35 mL) was added HF·Py (2.09 g, 70% purity, 10 equiv) solution at 0° C. The mixture was stirred at 0° C. for 0.5 hour. The mixture was quenched by sat. NaHCO₃ (100 mL) and extracted with ethyl acetate (2×50 mL). The organic layer was dried over Na₂SO₄ and concentrated to give a residue. The residue was purified by reversed phase flash chromatography [C18, 0.1% formic acid condition] to afford the title compound (3.80 g, 78% yield) as a yellow oil; LCMS (ESI, M+1): m/z=649.4.

Step C. ((3S,7aR)-7a-(((7-(8-ethyl-7-fluoro-3-(methoxymethoxy)naphthalen-1-yl)-8-fluoro-4-(2,2,2-trifluoroethoxy)pyrido[4,3-d]pyrimidin-2-yl)oxy)methyl)hexahydro-1H-pyrrolizin-3-yl)methyl dimethylcarbamate: To a solution of ((3S,7aR)-7a-(((7-(8-ethyl-7-fluoro-3-(methoxymethoxy)naphthalen-1-yl)-8-fluoro-4-(2,2,2-trifluoroethoxy)pyrido[4,3-d]pyrimidin-2-yl)oxy)methyl)hexahydro-1H-pyrrolizin-3-yl)methanol (2.60 g, 1.0 equiv) and DIPEA (3.11 g, 6.0 equiv) in ACN (26 mL) was added 4-nitrophenyl carbonochloridate (3.23 g, 4.0 equiv) at 0° C. The mixture was stirred at 50° C. for 4 hours. To the mixture was added N-methylmethanamine (1 M, 4.0 mL, 1.0 equiv) at 0° C. and the resulting was stirred at 0° C. for 0.5 hour. The mixture was diluted with water (200 mL) and extracted with ethyl acetate (2×100 mL). The organic layer was dried over Na₂SO₄ and concentrated to give a residue. The residue was purified by column chromatography [SiO₂, Petroleum ether/Ethyl acetate 5:1 to 1:1] and prep-HPLC [column: Unisil 3-100 C18 Ultra 150×50 mm×3 μm; A: water (FA), B: ACN, B %: 25%-55% over 7 min] to afford the title compound (700 mg, 22% yield) as a yellow oil; ¹H NMR (400 MHZ, CHLOROFORM-d) δ=9.25 (d, J=0.8 Hz, 1H), 7.68 (dd, J=5.6, 9.0 Hz, 1H), 7.53 (d, J=2.8 Hz, 1H), 7.30-7.19 (m, 2H), 5.34-5.24 (m, 2H), 5.11-4.83 (m, 2H), 4.45-4.27 (m, 1H), 4.16-3.96 (m, 1H), 3.90-3.71 (m, 2H), 3.52 (s, 3H), 3.13-2.97 (m, 1H), 2.89 (br s, 6H), 2.78-2.68 (m, 1H), 2.51-2.35 (m, 1H), 2.31-2.14 (m, 2H), 2.02-1.85 (m, 4H), 1.68-1.36 (m, 3H), 0.83 (q, J=7.6 Hz, 3H); LCMS (ESI, M−1): m/z=720.4.

Step D. ((3S,7aR)-7a-(((4-(4,6-dioxohexahydropyrrolo[3,4-c]pyrrol-2(1H)-yl)-7-(8-ethyl-7-fluoro-3-(methoxymethoxy)naphthalen-1-yl)-8-fluoropyrido[4,3-d]pyrimidin-2-yl)oxy)methyl)hexahydro-1H-pyrrolizin-3-yl)methyl dimethylcarbamate: To a mixture of ((3S,7aR)-7a-(((7-(8-ethyl-7-fluoro-3-(methoxymethoxy)naphthalen-1-yl)-8-fluoro-4-(2,2,2-trifluoroethoxy)pyrido[4,3-d]pyrimidin-2-yl)oxy)methyl)hexahydro-1H-pyrrolizin-3-yl)methyl dimethylcarbamate (80.0 mg, 1.0 equiv), tetrahydropyrrolo[3,4-c]pyrrole-1,3(2H,3aH)-dione (31.2 mg, 2.0 equiv) and 4 Å molecular sieves (20 mg) in DMF (0.1 mL) was added DIPEA (43.1 mg, 3 equiv). The reaction was stirred at 90° C. for 24 hours. The mixture was filtered and purified by reversed phase flash chromatography [C18, 0.1% formic acid condition] to afford the title compound (50.0 mg, 59% yield) as a yellow solid; LCMS (ESI, M+1): m/z=760.4.

Step E. ((3S,7aR)-7a-(((4-(4,6-dioxohexahydropyrrolo[3,4-c]pyrrol-2(1H)-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate: To a solution of ((3S,7aR)-7a-(((4-(4,6-dioxohexahydropyrrolo[3,4-c]pyrrol-2(1H)-yl)-7-(8-ethyl-7-fluoro-3-(methoxymethoxy)naphthalen-1-yl)-8-fluoropyrido[4,3-d]pyrimidin-2-yl)oxy)methyl)hexahydro-1H-pyrrolizin-3-yl)methyl dimethylcarbamate (50.0 mg, 1.0 equiv) in ACN (0.5 mL) was added HCl·dioxane (4 M, 0.5 mL, 30 equiv) at 0° C. The reaction was stirred at 0° C. for 0.5 hour. The mixture was concentrated, and the pH was adjusted to 8 by sat. NaHCO₃. The mixture was extracted with ethyl acetate (2×10 mL). The organic layer was dried over Na₂SO₄ and concentrated to give a residue. The residue was purified by reversed phase flash chromatography [C18, 0.1% formic acid condition] and prep-HPLC [column: Unisil 3-100 C18 Ultra 150×50 mm×3 μm; A: water (FA), B: ACN, B %: 15%-45% over 7 min] and [column: Waters Xbridge 150×25 mm×5 μm; A: water (NH₄HCO₃), B: ACN, B %: 40%-70% over 8 min] to afford the title compound (5.37 mg, 11% yield) as a white solid; ¹H NMR (400 MHZ, METHANOL-d₄) δ=8.99 (d, J=2.8 Hz, 1H), 7.67 (dd, J=6.0, 9.2 Hz, 1H), 7.30 (d, J=2.8 Hz, 1H), 7.25 (t, J=9.2 Hz, 1H), 7.12-7.01 (m, 1H), 4.45-4.31 (m, 1H), 4.17-4.01 (m, 2H), 3.94 (br t, J=10.4 Hz, 1H), 3.75-3.57 (m, 3H), 3.56-3.44 (m, 3H), 2.93-2.77 (m, 7H), 2.71-2.51 (m, 2H), 2.48-2.33 (m, 1H), 2.28-2.12 (m, 1H), 2.06-1.93 (m, 1H), 1.90-1.49 (m, 6H), 1.38-1.17 (m, 1H), 0.86-0.71 (m, 3H); LCMS (ESI, M+1): m/z=716.4.

### Example 709

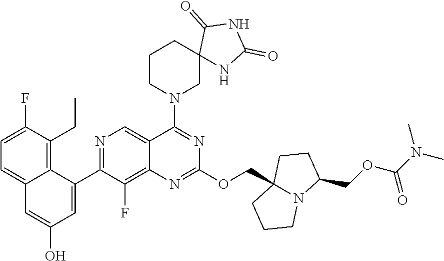 [SMILES: CCc1c(F)ccc2cc(O)cc(-c3ncc4c(N5CCCC6(C5)NC(=O)NC6=O)nc(OC[C@]56CCCN5[C@H](COC(=O)N(C)C)CC6)nc4c3F)c12]

### ((3S,7aR)-7a-(((4-(2,4-dioxo-1,3,7-triazaspiro[4.5]decan-7-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate

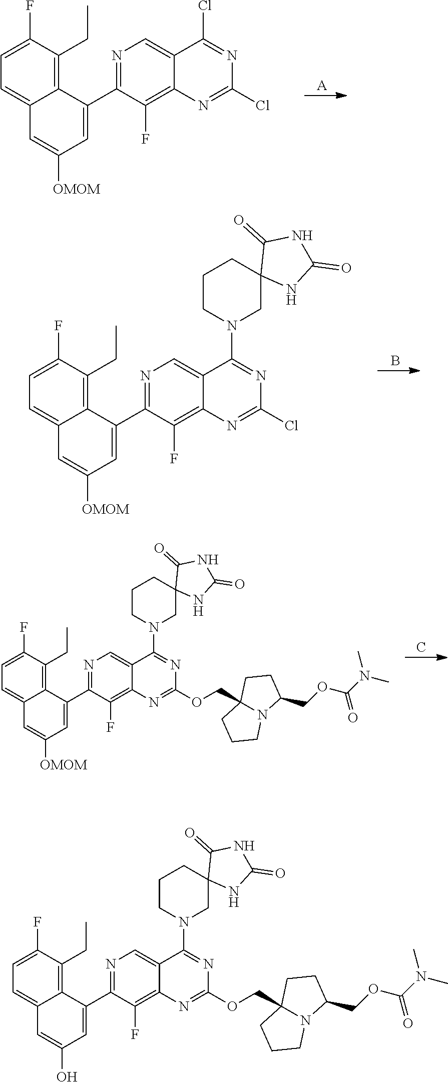

Step A. 7-(2-chloro-7-(8-ethyl-7-fluoro-3-(methoxymethoxy)naphthalen-1-yl)-8-fluoropyrido[4,3-d]pyrimidin-4-yl)-1,3,7-triazaspiro[4.5]decane-2,4-dione: To a solution of 2,4-dichloro-7-(8-ethyl-7-fluoro-3-(methoxymethoxy)naphthalen-1-yl)-8-fluoropyrido[4,3-d]pyrimidine (600 mg, 1.0 equiv) and DIPEA (517 mg, 3.0 equiv) in DCM (10 mL) was added 1,3,7-triazaspiro[4.5]decane-2,4-dione (203 mg, 0.9 equiv) at −40° C. The mixture was stirred at −40° C. for 0.5 hour. The mixture was diluted with water (10 mL) and extracted with dichloromethane (10 mL). The organic layer was dried over Na₂SO₄ and concentrated to give a residue. The residue was purified by reversed phase flash chromatography [C18, 0.1% formic acid condition] to afford the title compound (100 mg, 12% yield) as a yellow solid; LCMS (ESI, M+1): m/z=583.0.

Step B. ((3S,7aR)-7a-(((4-(2,4-dioxo-1,3,7-triazaspiro[4.5]decan-7-yl)-7-(8-ethyl-7-fluoro-3-(methoxymethoxy)naphthalen-1-yl)-8-fluoropyrido[4,3-d]pyrimidin-2-yl)oxy)methyl)hexahydro-1H-pyrrolizin-3-yl)methyl dimethylcarbamate: To a solution of ((3S,7aR)-7a-(hydroxymethyl)hexahydro-1H-pyrrolizin-3-yl)methyl dimethylcarbamate (57.4 mg, 1.5 equiv) in THF (2 mL) was added t-BuONa (2 M, 0.24 mL, 3.0 equiv) at 0° C. The mixture was stirred at 0° C. for 0.5 hour. To the mixture was added 7-(2-chloro-7-(8-ethyl-7-fluoro-3-(methoxymethoxy)naphthalen-1-yl)-8-fluoropyrido[4,3-d]pyrimidin-4-yl)-1,3,7-triazaspiro[4.5]decane-2,4-dione (92.0 mg, 1.0 equiv) at 0° C. The mixture was stirred at 0° C. for 0.5 hour. The mixture was quenched by water (5 mL) and extracted with ethyl acetate (10 mL). The organic phase was dried over Na₂SO₄ and concentrated to give a residue. The residue was purified by reversed phase flash chromatography [C18, 0.1% formic acid condition] to afford the title compound (36.0 mg, 29% yield) as a yellow solid; LCMS (ESI, M+1): m/z=789.4.

Step C. ((3S,7aR)-7a-(((4-(2,4-dioxo-1,3,7-triazaspiro[4.5]decan-7-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate: To a solution of ((3S,7aR)-7a-(((4-(2,4-dioxo-1,3,7-triazaspiro[4.5]decan-7-yl)-7-(8-ethyl-7-fluoro-3-(methoxymethoxy)naphthalen-1-yl)-8-fluoropyrido[4,3-d]pyrimidin-2-yl)oxy)methyl)hexahydro-1H-pyrrolizin-3-yl)methyl dimethylcarbamate (30.0 mg, 1.0 equiv) in ACN (0.5 mL) was added HCl·dioxane (4 M, 1 mL, 105 equiv) at 0° C. The mixture was stirred at 0° C. for 20 minutes. The mixture was concentrated and pH was adjusted to 9 by sat. NaHCO₃. The mixture was extracted with ethyl acetate (2×15 mL). The organic layer was dried over Na₂SO₄, concentrated and purified by prep-HPLC [column: Waters Xbridge 150×25 mm×5 μm; A: water (NH₄HCO₃), B: ACN, B %: 36%-66% over 9 min] to afford the title compound (10.9 mg, 38 yield) as a white solid; ¹H NMR (400 MHZ, METHANOL-d₄) δ=9.15-9.05 (m, 1H), 7.68 (dd, J=6.0, 9.0 Hz, 1H), 7.35-7.18 (m, 2H), 7.07 (d, J=1.8 Hz, 1H), 4.72-4.56 (m, 1H), 4.52-4.20 (m, 3H), 4.11 (br d, J=5.2 Hz, 1H), 4.04-3.91 (m, 1H), 3.89-3.64 (m, 2H), 3.13 (br s, 2H), 2.91 (br d, J=12.8 Hz, 7H), 2.55-2.34 (m, 1H), 2.30-2.02 (m, 6H), 1.99-1.68 (m, 7H), 0.80 (q, J=7.6 Hz, 3H); LCMS (ESI, M−1): m/z=745.4.

### Example 710

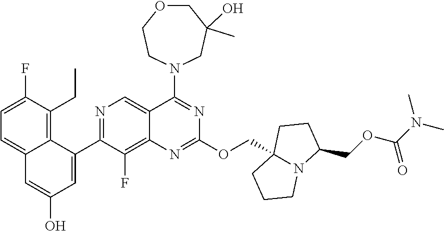 [SMILES: CCc1c(F)ccc2cc(O)cc(-c3ncc4c(N5CCOCC(C)(O)C5)nc(OC[C@@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-(6-hydroxy-6-methyl-1,4-oxazepan-4-yl)pyrido[4,3-d]pyrimidin-2-yl)oxy)methyl)hexahydro-1H-pyrrolizin-3-yl)methyl dimethylcarbamate

The title compound was synthesized using Intermediate 3A according to the 3-step procedure described for example 709 to produce the desired compound as a white solid; ¹H NMR (400 MHZ, METHANOL-d₄) δ=9.54 (br d, J=5.2 Hz, 1H), 7.72-7.61 (m, 1H), 7.30 (br s, 1H), 7.24 (br t, J=9.4 Hz, 1H), 7.13-7.00 (m, 1H), 4.64-4.41 (m, 2H), 4.34-4.14 (m, 3H), 4.10-3.83 (m, 5H), 3.75-3.61 (m, 2H), 3.10-2.77 (m, 9H), 2.57-2.40 (m, 1H), 2.21 (br dd, J=5.2, 11.2 Hz, 2H), 2.08-1.61 (m, 7H), 1.28 (br s, 3H), 0.81 (q, J=7.6 Hz, 3H); LCMS (ESI, M+1): m/z=707.2.

### Example 711

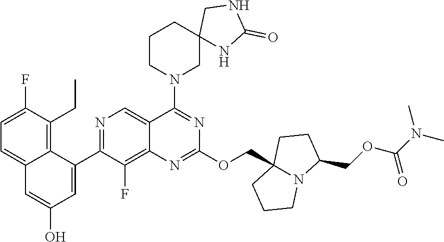 [SMILES: CCc1c(F)ccc2cc(O)cc(-c3ncc4c(N5CCCC6(CNC(=O)N6)C5)nc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-(2-oxo-1,3,7-triazaspiro[4.5]decan-7-yl)pyrido[4,3-d]pyrimidin-2-yl)oxy)methyl)hexahydro-1H-pyrrolizin-3-yl)methyl dimethylcarbamate

The title compound was synthesized using Intermediate 3 according to the 3-step procedure described for example 709, except for in Step A 4 Å MS (200 mg) were added and the mixture was stirred at 0° C. for 10 minutes to produce the desired compound as a white solid (0.65 formic acid salt).column: Chiralpak IC-3 50×4.6 mm I.D., 3 μm, mobile phase: [60% MeOH+ACN (0.05% DEA)] in CO₂, flow rate: 3 mL/min, detector: 220 nm, tR: 0.965 min; ¹H NMR (400 MHz, MeOH-d4) δ 9.22-9.05 (m, 1H), 7.69 (dd, J=5.6, 9.2 Hz, 1H), 7.31 (d, J=2.4 Hz, 1H), 7.29-7.22 (m, 1H), 7.10-7.04 (m, 1H), 4.62-4.43 (m, 2H), 4.39-4.12 (m, 4H), 4.06-3.86 (m, 2H), 3.54-3.41 (m, 2H), 3.26-3.17 (m, 1H), 2.99-2.79 (m, 6H), 2.54-2.31 (m, 2H), 2.25-1.83 (m, 14H), 0.89-0.74 (m, 3H); LCMS (ESI, M+1): m/z=731.5.

### Example 712

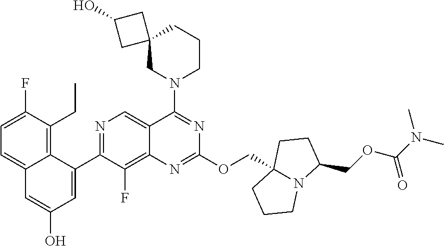 [SMILES: CCc1c(F)ccc2cc(O)cc(-c3ncc4c(N5CCC[C@]6(C5)C[C@H](O)C6)nc(OC[C@@]56CCCN5[C@H](COC(=O)N(C)C)CC6)nc4c3F)c12]

### ((3S,7aS)-7a-(((7-(8-ethyl-7-fluoro-3-hydroxynaphthalen-1-yl)-8-fluoro-4-((2R,4r)-2-hydroxy-6-azaspiro[3.5]nonan-6-yl)pyrido[4,3-d]pyrimidin-2-yl)oxy)methyl)hexahydro-1H-pyrrolizin-3-yl)methyl dimethylcarbamate

### Example 713

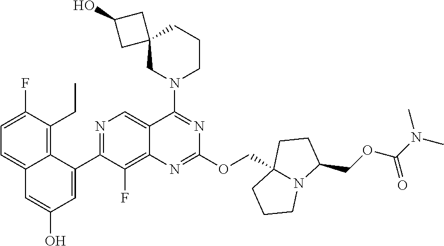 [SMILES: CCc1c(F)ccc2cc(O)cc(-c3ncc4c(N5CCC[C@]6(C5)C[C@@H](O)C6)nc(OC[C@@]56CCCN5[C@H](COC(=O)N(C)C)CC6)nc4c3F)c12]

### ((3S,7aS)-7a-(((7-(8-ethyl-7-fluoro-3-hydroxynaphthalen-1-yl)-8-fluoro-4-((2S,4s)-2-hydroxy-6-azaspiro[3.5]nonan-6-yl)pyrido[4,3-d]pyrimidin-2-yl)oxy)methyl)hexahydro-1H-pyrrolizin-3-yl)methyl dimethylcarbamate

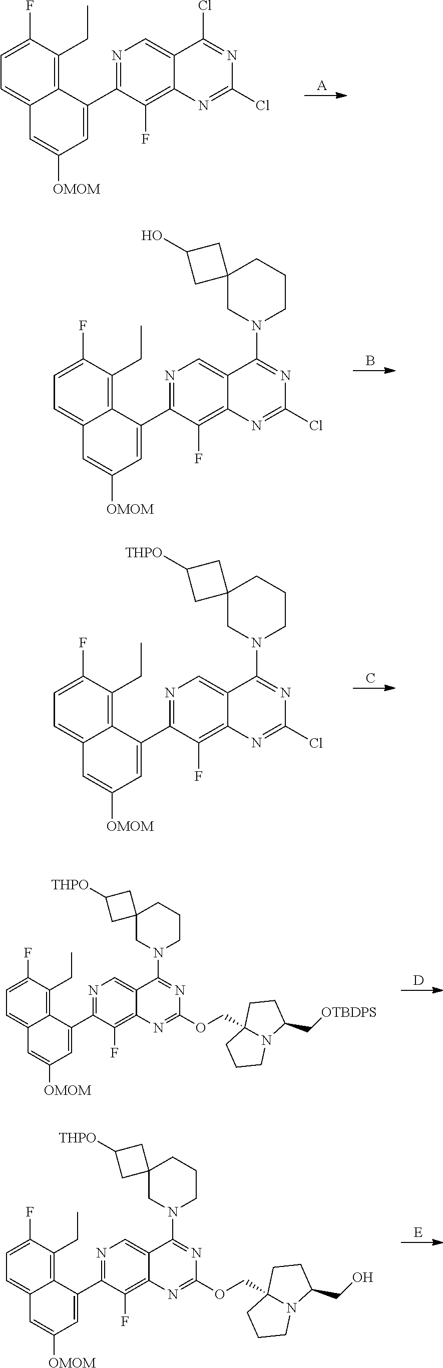

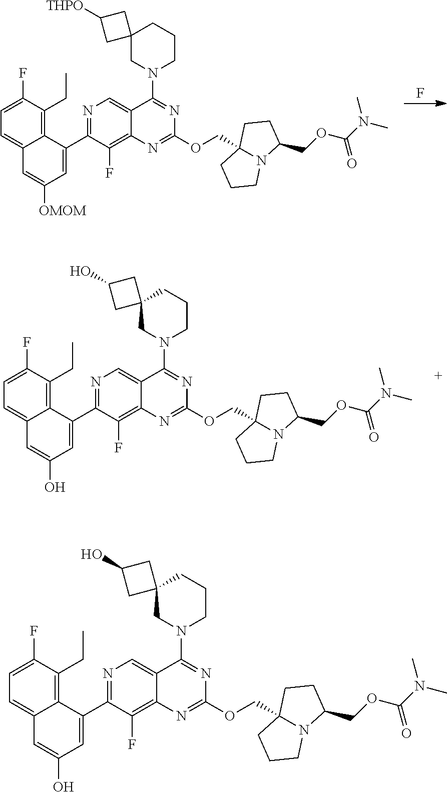

Step A. 6-(2-chloro-7-(8-ethyl-7-fluoro-3-(methoxymethoxy)naphthalen-1-yl)-8-fluoropyrido[4,3-d]pyrimidin-4-yl)-6-azaspiro[3.5]nonan-2-ol: To a solution of 2,4-dichloro-7-(8-ethyl-7-fluoro-3-(methoxymethoxy)naphthalen-1-yl)-8-fluoropyrido[4,3-d]pyrimidine (2.20 g, 1.0 equiv) and DIPEA (1.89 g, 3.0 equiv) in DCM (22 mL) was added 6-azaspiro[3.5]nonan-2-ol (1.03 g, 1.5 equiv) at −40° C. The mixture was stirred at −40° C. for 1.5 hours. The mixture was diluted with water (10 mL) and extracted with EtOAc (3×10 mL). The combined organic layers were dried over anhydrous sodium sulfate, concentrated and purified by reversed phase flash chromatography [C18, 0.1% formic acid condition] to afford the title compound (2.0 g, 73% yield) as a yellow solid; LCMS (ESI, M+1): m/z=555.2.

Step B. 2-chloro-7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidine: To a solution of 6-(2-chloro-7-(8-ethyl-7-fluoro-3-(methoxymethoxy)naphthalen-1-yl)-8-fluoropyrido[4,3-d]pyrimidin-4-yl)-6-azaspiro[3.5]nonan-2-ol (1.0 g, 1.0 equiv), TsOH (15.5 mg, 0.05 equiv) in THF (10 mL) was added DHP (455 mg, 3.0 equiv) at 0° C. The mixture was stirred at 20° C. for 2 hours. The mixture was quenched with saturated Na₂HCO₃ aqueous solution (20 mL) and extracted with EtOAc (3×10 mL). The combined organic layers were dried over anhydrous sodium sulfate, concentrated and purified by reversed phase flash chromatography [C18, 0.1% formic acid condition] to afford the title compound (1.0 g, 86% yield) as a yellow solid; LCMS (ESI, M+1): m/z=639.4.

Step C. 2-(((3S,7aS)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidine: To a mixture of ((3S,7aS)-3-(((tert-butyldiphenylsilyl)oxy)methyl)hexahydro-1H-pyrrolizin-7a-yl)methanol (769 mg, 3.0 equiv), 4 Å molecular sieves (80 mg) in THF (4 mL) was added t-BuONa (2 M, 3.0 equiv) at 0° C. The mixture was stirred at 20° C. for 0.5 hour. Then 2-chloro-7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidine (400 mg, 1.0 equiv) was added to the mixture at 0° C. The mixture was stirred at 20° C. for 0.5 hour. The mixture was diluted with water (10 mL) and extracted with EtOAc (3×10 mL). The combined organic layers were dried over anhydrous sodium sulfate, concentrated and purified by reversed phase flash chromatography [C18, 0.1% formic acid condition] to afford the title compound (150 mg, 21% yield) as a yellow solid; LCMS (ESI, M+1): m/z=1012.6.

Step D. ((3S,7aS)-7a-(((7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl)hexahydro-1H-pyrrolizin-3-yl)methanol: To a solution of 2-(((3S,7aS)-3-(((tert-butyldiphenylsilyl)oxy)methyl)hexahydro-1H-pyrrolizin-7a-yl)methoxy)-7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidine (150 mg, 1.0 equiv) in DMF (1 mL) was added CsF (225 mg, 10 equiv). The mixture was stirred at 40° C. for 12 hours. The mixture was filtered and washed with DMF (1 mL) and purified by reversed phase flash chromatography [C18, 0.1% formic acid condition] to afford the title compound (100 mg, 74% yield)) as a yellow solid; LCMS (ESI, M+1): m/z=774.5.

Step E. ((3S,7aS)-7a-(((7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl)hexahydro-1H-pyrrolizin-3-yl)methyl dimethylcarbamate: To a solution of ((3S,7aS)-7a-(((7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl)hexahydro-1H-pyrrolizin-3-yl)methanol (100 mg, 1.0 equiv) in THF (1 mL) was added NaH (10.3 mg, 60% purity, 2.0 equiv) at 0° C. The mixture was stirred at 0° C. for 0.5 hour. Then dimethylcarbamic chloride (34.7 mg, 2.5 equiv) was added to the mixture. The reaction was stirred at 0° C. for 1 hour. The mixture was diluted with water (10 mL) and extracted with EtOAc (3×10 mL). The combined organic layers were dried over anhydrous sodium sulfate, concentrated and purified by reversed phase flash chromatography [C18, 0.1% formic acid condition] to afford the title compound (40.0 mg, 37% yield) as a yellow solid; LCMS (ESI, M+1): m/z=845.5.

Step F. ((3S,7aS)-7a-(((7-(8-ethyl-7-fluoro-3-hydroxynaphthalen-1-yl)-8-fluoro-4-((2R,4r)-2-hydroxy-6-azaspiro[3.5]nonan-6-yl)pyrido[4,3-d]pyrimidin-2-yl)oxy)methyl)hexahydro-1H-pyrrolizin-3-yl)methyl dimethylcarbamate and ((3S,7aS)-7a-(((7-(8-ethyl-7-fluoro-3-hydroxynaphthalen-1-yl)-8-fluoro-4-((2S,4s)-2-hydroxy-6-azaspiro[3.5]nonan-6-yl)pyrido[4,3-d]pyrimidin-2-yl)oxy)methyl)hexahydro-1H-pyrrolizin-3-yl)methyl dimethylcarbamate: To a solution of ((3S,7aS)-7a-(((7-(8-ethyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl)hexahydro-1H-pyrrolizin-3-yl)methyl dimethylcarbamate (50.0 mg, 1.0 equiv) in MeOH (0.5 mL) was added TsOH (204 mg, 20 equiv). The mixture was stirred at 20° C. for 12 hours. The resulting mixture was filtered, washed with MeOH (1 mL) and the filtrate was purified with prep-HPLC [Waters Xbridge 150×25 mm×5 μm; A: water (10 mM NH₄HCO₃), B: ACN; B %: 48%-78% over 8 min] and lyophilized to afford two peaks. EXAMPLE 712 (5.80 mg, 13% yield) as white solid; ¹H NMR (400 MHZ, METHANOL-d₄) δ=9.13-8.95 (m, 1H), 7.68 (dd, J=5.6, 9.2 Hz, 1H), 7.30 (d, J=2.4 Hz, 1H), 7.25 (t, J=9.2 Hz, 1H), 7.08 (dd, J=2.4, 5.2 Hz, 1H), 4.45-4.19 (m, 3H), 4.18-4.01 (m, 3H), 4.00-3.85 (m, 3H), 3.15-2.99 (m, 2H), 2.92 (br d, J=17.2 Hz, 6H), 2.88-2.77 (m, 1H), 2.57-2.43 (m, 1H), 2.36-2.13 (m, 4H), 2.07-2.00 (m, 1H), 1.98-1.85 (m, 3H), 1.85-1.75 (m, 6H), 1.74-1.63 (m, 3H), 0.81 (t, J=7.2 Hz, 3H); LCMS (ESI, M+1): m/z=717.4. EXAMPLE 713 (5.80 mg, 13% yield) as a white solid; ¹H NMR (400 MHZ, METHANOL-d₄) δ=9.05 (s, 1H), 7.67 (dd, J=5.6, 8.8 Hz, 1H), 7.30 (d, J=2.0 Hz, 1H), 7.25 (t, J=9.2 Hz, 1H), 7.07 (br s, 1H), 4.36-4.18 (m, 3H), 4.15-3.89 (m, 6H), 3.11-3.00 (m, 2H), 2.92 (br d, J=18.4 Hz, 6H), 2.84 (br dd, J=5.2, 10.4 Hz, 1H), 2.57-2.42 (m, 1H), 2.29-2.14 (m, 4H), 2.08-2.00 (m, 1H), 1.97-1.88 (m, 3H), 1.87-1.78 (m, 6H), 1.77-1.63 (m, 3H), 0.82 (t, J=7.2 Hz, 3H); LCMS (ESI, M+1): m/z=717.4.

### Example 714

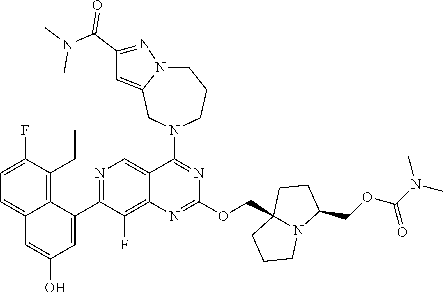 [SMILES: CCc1c(F)ccc2cc(O)cc(-c3ncc4c(N5CCCn6nc(C(=O)N(C)C)cc6C5)nc(OC[C@]56CCCN5[C@H](COC(=O)N(C)C)CC6)nc4c3F)c12]

### ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate

The title compound was synthesized using Intermediate 3 according to the 3-step procedure described for example 709 to produce the desired compound as a white solid (0.16 formic acid salt); ¹H NMR (400 MHZ, METHANOL-d₄) δ=9.17 (s, 1H), 7.67 (dd, J=6.0, 9.0 Hz, 1H), 7.30 (d, J=2.8 Hz, 1H), 7.24 (t, J=9.2 Hz, 1H), 7.05 (t, J=2.4 Hz, 1H), 6.75 (s, 1H), 5.41-5.16 (m, 2H), 4.55 (br d, J=6.0 Hz, 2H), 4.50-4.35 (m, 2H), 4.31 (s, 2H), 4.13 (br dd, J=3.0, 6.4 Hz, 1H), 4.08-3.97 (m, 1H), 3.31 (s, 3H), 3.16 (br d, J=5.6 Hz, 2H), 3.07 (s, 3H), 3.00-2.81 (m, 7H), 2.53-2.35 (m, 3H), 2.29-2.11 (m, 2H), 2.08-1.90 (m, 4H), 1.89-1.70 (m, 3H), 0.78 (t, J=7.4 Hz, 3H); LCMS (ESI, M+1): m/z=784.5.

### Example 715

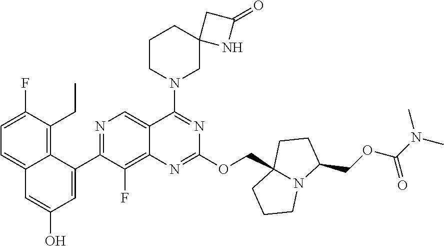 [SMILES: CCc1c(F)ccc2cc(O)cc(-c3ncc4c(N5CCCC6(CC(=O)N6)C5)nc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-(2-oxo-1,6-diazaspiro[3.5]nonan-6-yl)pyrido[4,3-d]pyrimidin-2-yl)oxy)methyl)hexahydro-1H-pyrrolizin-3-yl)methyl dimethylcarbamate

The title compound was synthesized using Intermediate 3 according to the 3-step procedure described for example 709, except that TFA-DCM (1:1) was used in Step C, to produce the desired compound as a white solid (0.25 formic acid salt); ¹H NMR (400 MHZ, METHANOL-d₄) δ=9.12-9.04 (m, 1H), 7.70-7.66 (m, 1H), 7.33-7.20 (m, 2H), 7.06 (br s, 1H), 4.44-4.31 (m, 4H), 4.18-4.11 (m, 1H), 4.09-3.97 (m, 2H), 3.85-3.72 (m, 1H), 3.19 (br d, J=1.2 Hz, 2H), 2.99-2.84 (m, 8H), 2.79-2.72 (m, 1H), 2.51-2.40 (m, 1H), 2.30-2.22 (m, 1H), 2.18-1.99 (m, 6H), 1.95-1.71 (m, 6H), 0.79 (br t, J=7.2 Hz, 3H); LCMS (ESI, M+1): m/z=716.5.

### Example 716

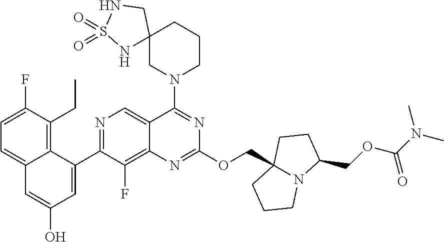 [SMILES: CCc1c(F)ccc2cc(O)cc(-c3ncc4c(N5CCCC6(CNS(=O)(=O)N6)C5)nc(OC[C@]56CCCN5[C@H](COC(=O)N(C)C)CC6)nc4c3F)c12]

### ((3S,7aR)-7a-(((4-(2,2-dioxido-2-thia-1,3,7-triazaspiro[4.5]decan-7-yl)-7-(8-ethyl-7-fluoro-3-hydroxynaphthalen-1-yl)-8-fluoropyrido[4,3-d]pyrimidin-2-yl)oxy)methyl)hexahydro-1H-pyrrolizin-3-yl)methyl dimethylcarbamate

The title compound was synthesized using Intermediate 3 according to the 3-step procedure described for example 709, except that for Step B NaH (3 equiv) in THF and stirring at 25° C. for 12 hours were used, to produce the desired compound as a white solid; ¹H NMR (400 MHz, METHANOL-d₄) δ=9.08 (s, 1H), 7.68-7.65 (m, 1H), 7.29 (d, J=2.8 Hz, 1H), 7.24 (t, J=9.2 Hz, 1H), 7.05 (t, J=2.4 Hz, 1H), 4.52-4.48 (m, 1H), 4.31-4.23 (m, 3H), 4.31 (s, 2H), 4.06-3.99 (m, 1H), 3.81-3.62 (s, 1H), 3.38-3.33 (s, 1H), 3.18-3.16 (m, 1H), 3.10-3.01 (s, 2H), 2.92-2.81 (m, 6H), 2.80-2.72 (m, 1H), 2.52-2.41 (m, 1H), 2.20-2.00 (m, 5H), 1.94-1.69 (m, 7H), 1.67-1.63 (m, 1H), 0.80 (t, J=7.2 Hz, 3H); LCMS (ESI, M+1): m/z=767.2.

### Example 717

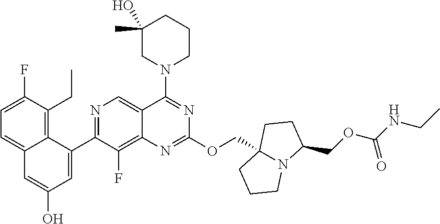 [SMILES: CCNC(=O)OC[C@@H]1CC[C@]2(COc3nc(N4CCC[C@@](C)(O)C4)c4cnc(-c5cc(O)cc6ccc(F)c(CC)c56)c(F)c4n3)CCCN12]

### ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl ethylcarbamate

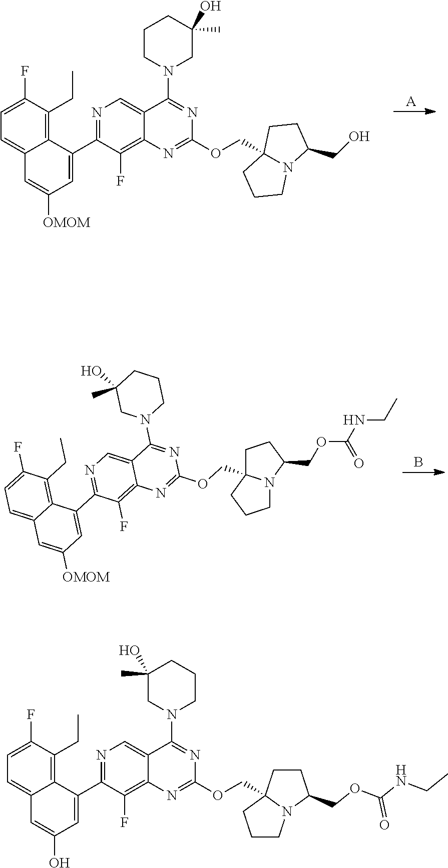

Step A. ((3S,7aS)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl ethylcarbamate: To a solution of (R)-1-(7-(8-ethyl-7-fluoro-3-(methoxymethoxy)naphthalen-1-yl)-8-fluoro-2-(((3S,7aS)-3-(hydroxymethyl)hexahydro-1H-pyrrolizin-7a-yl)methoxy)pyrido[4,3-d]pyrimidin-4-yl)-3-methylpiperidin-3-ol (100 mg, 1.0 equiv) and isocyanatoethane (53.5 mg, 5.0 equiv) in MeCN (2 mL) was added DABCO (5.07 mg, 0.3 equiv). The mixture was stirred at 20° C. for 12 hours. The mixture was filtered and washed with MeCN (1 mL). The filtrate was concentrated and purified by reversed phase flash chromatography [C18, 0.1% formic acid condition] to afford the title compound (90.0 mg, 81% yield) as a yellow solid; LCMS (ESI, M+1): m/z=735.5.

Step B. ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl ethylcarbamate: To a solution of ((3S,7aS)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl ethylcarbamate (90.0 mg, 1.0 equiv) in MeOH (0.25 mL) was added HCl·MeOH (4 M, 4.50 mL, 147 equiv). The mixture was stirred at 0° C. for 0.5 hour. The pH of the mixture was adjusted to 8 with with saturated NaHCO₃ aqueous solution (3 mL) and extracted with ethyl acetate (3×5 mL). The combined organic layers were dried over anhydrous sodium sulfate, concentrated and purified with prep-HPLC [Waters Xbridge 150×25 mm×5 μm; A: water (10 mM NH₄HCO₃), B: ACN; B %: 45%-75% over 9 min] and lyophilized to afford the title compound (22.9 mg, 27% yield) as a white solid; ¹H NMR (400 MHZ, METHANOL-d₄) δ=9.20 (s, 1H), 7.67 (dd, J=6.0, 9.2 Hz, 1H), 7.30 (d, J=2.4 Hz, 1H), 7.25 (t, J=9.2 Hz, 1H), 7.06 (d, J=2.4 Hz, 1H), 4.53 (br d, J=13.6 Hz, 1H), 4.37-4.21 (m, 3H), 4.10-3.93 (m, 2H), 3.71-3.55 (m, 1H), 3.53-3.41 (m, 1H), 3.12 (q, J=7.2 Hz, 2H), 3.08-2.96 (m, 2H), 2.89-2.79 (m, 1H), 2.56-2.38 (m, 1H), 2.27-2.11 (m, 3H), 2.07-1.99 (m, 1H), 1.97-1.84 (m, 4H), 1.83-1.73 (m, 4H), 1.72-1.62 (m, 1H), 1.29 (d, J=10.4 Hz, 3H), 1.09 (t, J=7.2 Hz, 3H), 0.88-0.76 (m, 3H); LCMS (ESI, M+1): m/z=691.5.

### Example 718

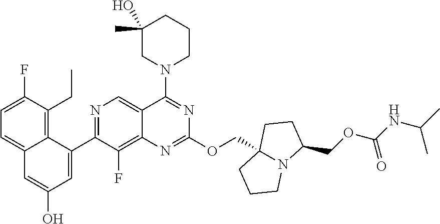 [SMILES: CCc1c(F)ccc2cc(O)cc(-c3ncc4c(N5CCC[C@@](C)(O)C5)nc(OC[C@@]56CCCN5[C@H](COC(=O)NC(C)C)CC6)nc4c3F)c12]

### ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl isopropylcarbamate

The title compound was synthesized from Intermediate 18 according to the 2-step procedure described for example 717 to produce the desired compound as a white solid; ¹H NMR (400 MHZ, METHANOL-d4) δ=9.20 (s, 1H), 7.67 (dd, J=5.6, 8.8 Hz, 1H), 7.30 (d, J=2.4 Hz, 1H), 7.25 (t, J=9.6 Hz, 1H), 7.06 (d, J=2.4 Hz, 1H), 4.53 (br d, J=13.2 Hz, 1H), 4.36-4.20 (m, 3H), 4.07-3.92 (m, 2H), 3.76-3.56 (m, 2H), 3.53-3.41 (m, 1H), 3.11-2.95 (m, 2H), 2.90-2.78 (m, 1H), 2.55-2.41 (m, 1H), 2.27-2.12 (m, 3H), 2.08-1.99 (m, 1H), 1.98-1.84 (m, 4H), 1.83-1.72 (m, 4H), 1.72-1.62 (m, 1H), 1.29 (d, J=10.6 Hz, 3H), 1.12 (d, J=6.4 Hz, 6H), 0.81 (q, J=7.2 Hz, 3H); LCMS (ESI, M+1): m/z=705.5.

### Example 719

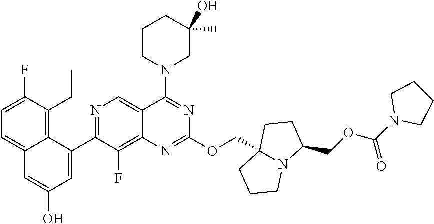 [SMILES: CCc1c(F)ccc2cc(O)cc(-c3ncc4c(N5CCC[C@@](C)(O)C5)nc(OC[C@@]56CCCN5[C@H](COC(=O)N5CCCC5)CC6)nc4c3F)c12]

### ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl pyrrolidine-1-carboxylate

The title compound was synthesized from Intermediate 18 according to the 2-step procedure described for example 706 to produce the desired compound as a white solid; SFC: Chiralcel OJ-3 50×4.6 mm I.D., 3 μm, MeOH (0.05% DEA) in CO₂ from 5% to 40% Flow rate: 3 mL/min, detector: 220 nm, t_{R1}=1.461 min; ¹H NMR (400 MHZ, CHLOROFORM-d) δ=9.20-9.00 (m, 1H), 7.60-7.44 (m, 1H), 7.24-7.12 (m, 2H), 7.04-6.92 (m, 1H), 4.56-4.32 (m, 3H), 4.20 (br d, J=10.4 Hz, 1H), 4.12-3.94 (m, 2H), 3.44-3.24 (m, 6H), 3.24-3.08 (m, 1H), 3.04-2.92 (m, 2H), 2.80-2.68 (m, 1H), 2.64-2.40 (m, 1H), 2.28-1.96 (m, 5H), 1.96-1.84 (m, 3H), 1.80-1.54 (m, 8H), 1.36-1.28 (m, 3H), 0.83 (td, J=7.4, 14.6 Hz, 3H); LCMS (ESI, M+1): m/z=717.5.

### Example 720

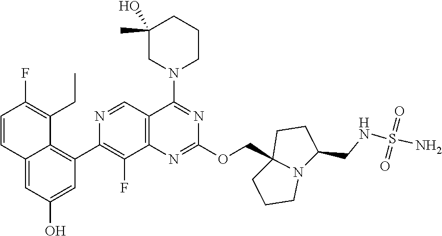 [SMILES: CCc1c(F)ccc2cc(O)cc(-c3ncc4c(N5CCC[C@@](C)(O)C5)nc(OC[C@]56CCCN5[C@H](CNS(N)(=O)=O)CC6)nc4c3F)c12]

### N,N-dimethyl-(((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl)aminosulfonamide

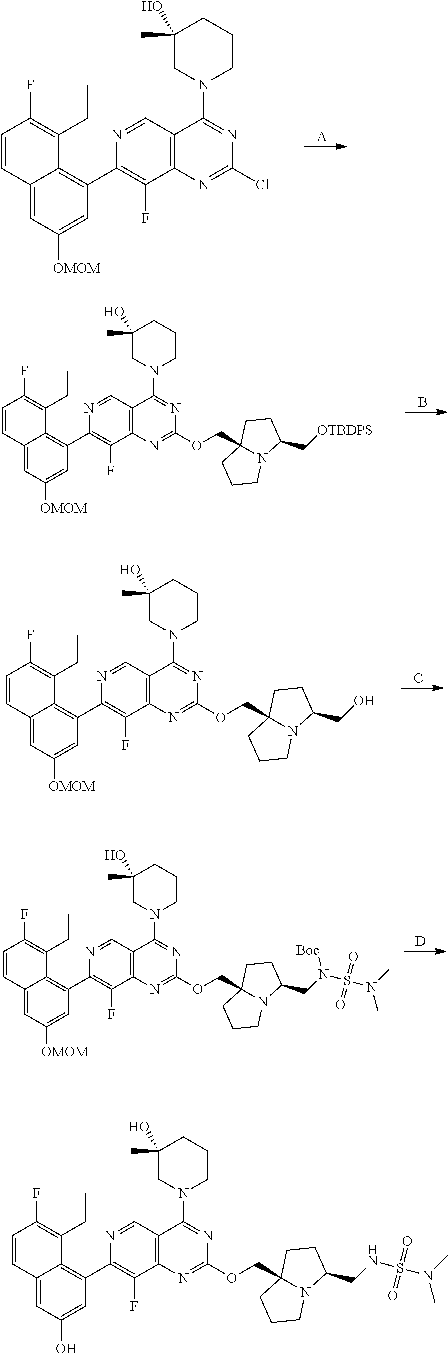

Step A. (R)-1-(2-(((3S,7aR)-3-(((tert-butyldiphenylsilyl)oxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: A solution of ((3S,7aR)-3-(((tert-butyldiphenylsilyl)oxy)methyl)tetrahydro-1H-pyrrolizin-7a(5H)-yl)methanol (851.8 mg, 1.1 equiv), 4 Å MS (100 mg) and t-BuONa (545 mg, 3.0 equiv) in toluene (10 mL) was stirred at 0° C. for 10 mins. Then (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (1.00 g, 1.0 equiv) was added into the mixture and the mixture was stirred at 0° C. for 1 hour. The reaction mixture was quenched with H₂O (30 mL) at 0° C. and extracted with ethyl acetate (3×30 mL). Then the combined organic phases were washed with brine (50 mL), dried over anhydrous sodium sulfate, filtered and concentrated under vacuum. The residue was purified by reversed phase flash chromatography [C8, 0.1% formic acid condition]. The desired fractions were collected and neutralized with solid NaHCO₃ and concentrated under vacuum to remove acetonitrile. The aqueous layer was extracted with ethyl acetate (3×100 mL). The combined organic layers were dried over anhydrous sodium sulfate and concentrated under vacuum to afford the title compound (1.2 g, 69% yield) as a yellow oil. ¹H NMR (400 MHZ, METHANOL-d₄) δ=9.20 (d, J=6.0 Hz, 1H), 7.79 (dd, J=6.0, 9.0 Hz, 1H), 7.74-7.66 (m, 4H), 7.62 (d, J=2.8 Hz, 1H), 7.44-7.34 (m, 6H), 7.31 (t, J=9.6 Hz, 1H), 7.22 (t, J=2.8 Hz, 1H), 5.33 (s, 2H), 4.50 (br d, J=13.2 Hz, 1H), 4.30-4.16 (m, 3H), 3.72-3.52 (m, 3H), 3.50 (s, 3H), 3.43-3.34 (m, 1H), 3.02-2.87 (m, 2H), 2.81-2.68 (m, 1H), 2.56-2.42 (m, 1H), 2.26-2.08 (m, 3H), 1.95-1.59 (m, 10H), 1.28-1.24 (m, 4H), 1.04 (d, J=1.2 Hz, 9H), 0.81 (td, J=7.2, 11.3 Hz, 3H); LCMS (ESI, M+1): m/z=902.4.

Step B. (R)-1-(7-(8-ethyl-7-fluoro-3-(methoxymethoxy)naphthalen-1-yl)-8-fluoro-2-(((3S,7aR)-3-(hydroxymethyl)tetrahydro-1H-pyrrolizin-7a(5H)-yl)methoxy)pyrido[4,3-d]pyrimidin-4-yl)-3-methylpiperidin-3-ol: To a solution of (R)-1-(2-(((3S,7aR)-3-(((tert-butyldiphenylsilyl)oxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (1.10 g, 1.0 equiv) in DMF (11 mL) was added CsF (2.80 g, 15.0 equiv). The mixture was stirred at 40° C. for 12 hours. The reaction mixture was filtered and the filtrate was collected. The crude product was purified by reversed phase flash chromatography [C8, 0.1% formic acid condition] to afford the title compound (690 mg, 82% yield) as a yellow solid. ¹H NMR (400 MHZ, CHLOROFORM-d) δ=9.13 (s, 1H), 8.38 (s, 1H), 7.67 (dd, J=5.6, 8.8 Hz, 1H), 7.6 (d, J=2.8 Hz, 1H), 7.26-7.19 (m, 2H), 5.32-5.27 (m, 2H), 4.77-4.46 (m, 4H), 4.41 (br t, J=12.6 Hz, 1H), 3.95 (d, J=4.0 Hz, 2H), 3.72-3.58 (m, 2H), 3.56-3.41 (m, 4H), 3.33 (dt, J=4.8, 10.1 Hz, 1H), 3.21 (qd, J=5.6, 11.4 Hz, 1H), 2.54 (ddd, J=2.8, 6.8, 13.2 Hz, 2H), 2.25-2.07 (m, 7H), 1.96-1.85 (m, 2H), 1.80-1.62 (m, 2H), 1.38 (s, 3H), 0.85 (dt, J=4.8, 7.3 Hz, 3H); LCMS (ESI, M+1): m/z=664.4.

Step C. tert-butyl (N,N-dimethylsulfamoyl) (((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl)carbamate: To a solution of (R)-1-(7-(8-ethyl-7-fluoro-3-(methoxymethoxy)naphthalen-1-yl)-8-fluoro-2-(((3S,7aR)-3-(hydroxymethyl)tetrahydro-1H-pyrrolizin-7a(5H)-yl)methoxy)pyrido[4,3-d]pyrimidin-4-yl)-3-methylpiperidin-3-ol (50.0 mg, 1.0 equiv) in THF (1.5 mL) was tert-butyl(N,N-dimethylsulfamoyl)carbamate (33.8 mg, 2.0 equiv), PPh₃ (39.5 mg, 2.0 equiv) and DIAD (30.5 mg, 2.0 equiv) at 0° C. under N₂. The mixture was stirred at 20° C. for 12 hours. The reaction mixture was concentrated and purified with prep-HPLC [C18, 0.1% formic acid condition] and lyophilized to afford the title compound (20 mg, 27.5% yield) as a white solid. ¹H NMR (400 MHz, DMSO-d₆) δ=9.24 (d, J=5.2 Hz, 1H), 7.90 (dd, J=6.0, 9.1 Hz, 1H), 7.68 (d, J=2.4 Hz, 1H), 7.44 (t, J=9.6 Hz, 1H), 7.24 (d, J=2.8 Hz, 1H), 5.35 (s, 2H), 4.80-4.70 (m, 1H), 4.35 (br t, J=13.6 Hz, 1H), 4.19-3.98 (m, 3H), 3.67-3.49 (m, 3H), 3.44 (s, 3H), 2.96-2.84 (m, 2H), 2.81 (d, J=1.6 Hz, 6H), 2.69-2.64 (m, 1H), 2.42-2.31 (m, 1H), 2.24-2.10 (m, 1H), 2.07-1.96 (m, 2H), 1.95-1.86 (m, 1H), 1.84-1.76 (m, 3H), 1.75-1.52 (m, 7H), 1.45 (d, J=3.2 Hz, 9H), 1.20-1.15 (m, 4H), 0.75 (q, J=7.2 Hz, 3H); LCMS (ESI, M+1): m/z=870.4.

Step D. N,N-dimethyl-(((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl)aminosulfonamide: To a solution of TFA (0.45 mL) in DCM (0.3 mL) was added a mixture of tert-butyl(N,N-dimethylsulfamoyl) (((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl)carbamate (15.0 mg, 1.0 equiv) in DCM (0.3 mL) at 0° C. The mixture was stirred at 25° C. for 2 hours. The pH of the reaction mixture was adjusted to pH=8 with 2 N NaOH aqueous solution, the resulting was extracted with DCM (3 mL×3). The combined organic layers were concentrated. The residue was purified with prep-HPLC [C18, 0.1% TFA condition] and lyophilized to afford the title compound (10 mg, 78% yield, TFA salt) as a white solid. ¹H NMR (400 MHZ, methanol-d₄) δ=9.25 (d, J=2.4 Hz, 1H), 8.45 (br s, 1H), 7.68 (dd, J=6.0, 8.9 Hz, 1H), 7.31 (d, J=2.4 Hz, 1H), 7.25 (t, J=9.6 Hz, 1H), 7.07 (dd, J=2.4, 11.4 Hz, 1H), 4.64-4.49 (m, 3H), 4.32 (br t, J=13.6 Hz, 1H), 3.69-3.55 (m, 1H), 3.53-3.38 (m, 3H), 3.36-3.32 (m, 1H), 3.30-3.22 (m, 2H), 2.75 (d, J=1.2 Hz, 6H), 2.51-2.32 (m, 2H), 2.27-2.08 (m, 6H), 2.07-1.93 (m, 3H), 1.91-1.73 (m, 3H), 1.35-1.23 (m, 3H), 0.82 (dt, J=4.0, 7.2 Hz, 3H); ¹⁹F NMR (376 MHz, methanol-d₄) δ=−77, −121, −139; LCMS (ESI, M+1): m/z=726.4.

### Example 721

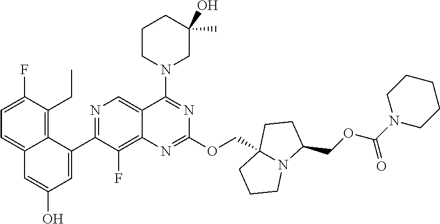 [SMILES: CCc1c(F)ccc2cc(O)cc(-c3ncc4c(N5CCC[C@@](C)(O)C5)nc(OC[C@@]56CCCN5[C@H](COC(=O)N5CCCCC5)CC6)nc4c3F)c12]

### ((3S,7aS)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl piperidine-1-carboxylate

The title compound was synthesized from Intermediate 18 according to the 2-step procedure described for example 706 to produce the desired compound as a white solid; SFC: Chiralpak IC-3 50×4.6 mm I.D., 3 μm, 40% MeOH+ACN (0.05% DEA) in CO₂ Flow rate: 3 mL/min, detector: 220 nm, t_{R1}=1.409 min; ¹H NMR (400 MHZ, CHLOROFORM-d) δ=9.25-9.03 (m, 1H), 7.64-7.44 (m, 1H), 7.24-7.08 (m, 2H), 7.08-6.80 (m, 1H), 4.64-4.28 (m, 3H), 4.24-4.12 (m, 1H), 4.12-3.76 (m, 2H), 3.56-3.24 (m, 7H), 3.20-2.92 (m, 2H), 2.88-2.64 (m, 1H), 2.64-2.40 (m, 1H), 2.32-1.96 (m, 5H), 1.94-1.64 (m, 9H), 1.62-1.56 (m, 2H), 1.36-1.16 (m, 5H), 0.88-0.76 (m, 3H); LCMS (ESI, M+1): m/z=731.5

### Example 722

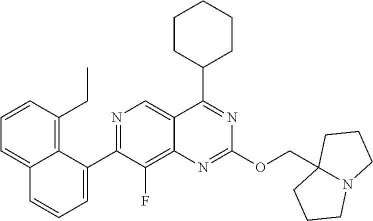 [SMILES: CCc1cccc2cccc(-c3ncc4c(C5CCCCC5)nc(OCC56CCCN5CCC6)nc4c3F)c12]

### 4-cyclohexyl-7-(8-ethylnaphthalen-1-yl)-8-fluoro-2-((hexahydro-1H-pyrrolizin-7a-yl)methoxy)pyrido[4,3-d]pyrimidine

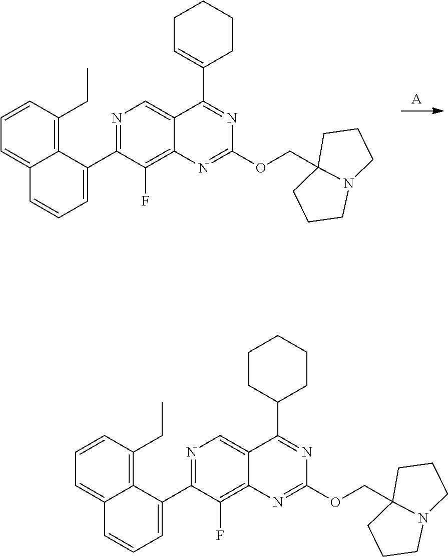

To a solution of 4-(cyclohex-1-en-1-yl)-7-(8-ethylnaphthalen-1-yl)-8-fluoro-2-((tetrahydro-1H-pyrrolizin-7a(5H)-yl)methoxy)pyrido[4,3-d]pyrimidine (synthesized via the same procedure as described for Example 524) (10 mg, 1.0 equiv) in EtOAc (2 mL) was added Pd/C (20 mg) under H₂ atmosphere. The suspension was degassed and purged with H₂ for 3 times. The mixture was stirred under H₂ atmosphere (15 Psi) at 25° C. for 15 min. The reaction mixture was filtered and concentrated under reduced pressure to give a residue. The residue was purified by prep-HPLC (column: Phenomenex luna C18 150×25 mm×10 um; mobile phase: [water (FA)-ACN]; B %: 24%-54%, 10 min) to afford the title compound (5 mg, 49% yield, 99.6% purity) as a yellow solid (0.26 formic acid salt). ¹H NMR (400 MHZ, METHANOL-d₄) δ=9.42 (s, 1H), 8.53 (br s, 1H), 8.06 (d, J=8.0 Hz, 1H), 7.88 (d, J=8.0 Hz, 1H), 7.57 (t, J=7.6 Hz, 1H), 7.53-7.47 (m, 1H), 7.45 (d, J=6.4 Hz, 1H), 7.41 (d, J=7.2 Hz, 1H), 4.46 (s, 2H), 3.88-3.75 (m, 1H), 3.24-3.16 (m, 2H), 2.86-2.79 (m, 2H), 2.40-2.25 (m, 2H), 2.14 (dd, J=6.4, 12.4 Hz, 2H), 2.09-2.05 (m, 1H), 2.02-1.78 (m, 12H), 1.70-1.59 (m, 2H), 1.47-1.37 (m, 1H), 0.89 (t, J=7.2 Hz, 3H). LCMS (ESI, M+1): m/z=525.3.

### Example 723

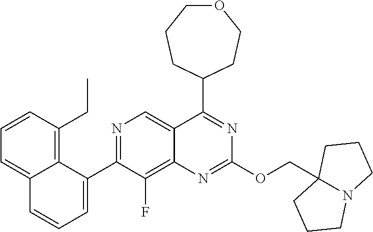 [SMILES: CCc1cccc2cccc(-c3ncc4c(C5CCCOCC5)nc(OCC56CCCN5CCC6)nc4c3F)c12]

### rac-(R)-7-(8-ethylnaphthalen-1-yl)-8-fluoro-4-(oxepan-4-yl)-2-((tetrahydro-1H-pyrrolizin-7a(5H)-yl)methoxy)pyrido[4,3-d]pyrimidine

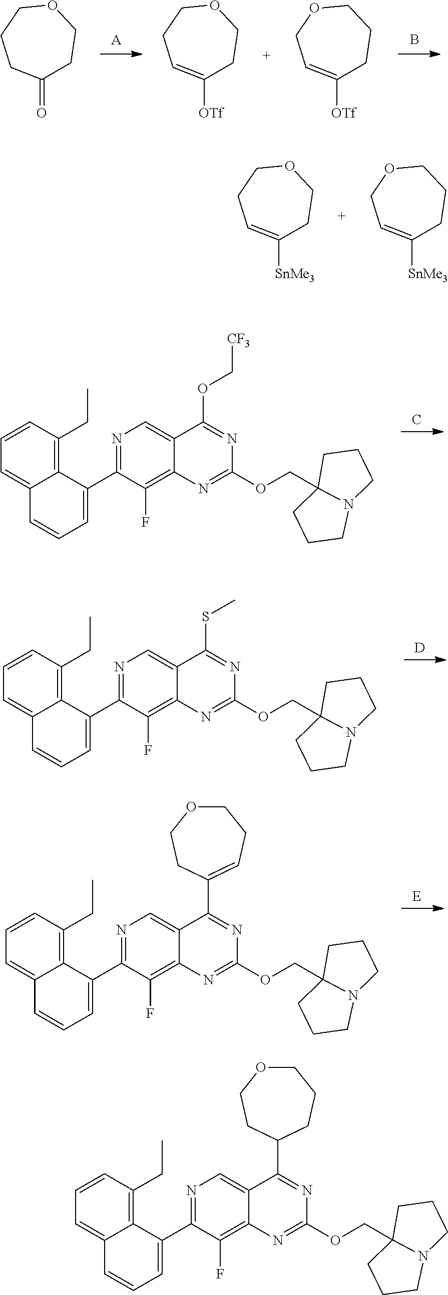

Step A. 2,3,4,7-tetrahydrooxepin-5-yl trifluoromethanesulfonate: To a solution of oxepan-4-one (200 mg, 1.0 equiv) in THF (3 mL) was added LDA (2 M, 1 mL, 1.2 equiv) at −78° C. under nitrogen atmosphere. The mixture was stirred at −78° C. for 0.5 hour. Then a solution of 1,1,1-trifluoro-N-phenyl-N-(trifluoromethylsulfonyl)methanesulfonamide (626 mg, 1.0 equiv) in THF (1 mL) was added. The resulting reaction mixture was stirred at 20° C. for 16 hours. The mixture was quenched with saturated ammonium chloride solution (30 mL) and extracted with EtOAc (10 mL×2). The organic layer was washed with brine (20 mL), dried over anhydrous sodium sulfate and concentrated. The residue was purified by column chromatography (SiO₂, petroleum ether/ethyl acetate 20:1 to 3:1) to afford the title compound (150 mg, mixture) as a light yellow oil; ¹H NMR (400 MHZ, CHLOROFORM-d) δ=7.46-7.38 (m, 2H), 7.36-7.27 (m, 3H), 6.76 (br s, 1H), 5.98 (t, J=6.0 Hz, 1H), 5.83 (t, J=4.4 Hz, 1H), 4.25-4.16 (m, 1H), 3.85 (t, J=5.6 Hz, 1H), 3.79 (t, J=5.2 Hz, 2H), 2.74 (t, J=5.2 Hz, 1H), 2.70-2.62 (m, 1H), 2.37 (q, J=5.4 Hz, 1H), 1.99 (td, J=5.9, 11.8 Hz, 1H)

Step B. trimethyl (2,3,6,7-tetrahydrooxepin-4-yl) stannane: To a solution of 2,3,6,7-tetrahydrooxepin-4-yl trifluoromethanesulfonate (100 mg, 1.0 equiv) and trimethyl(trimethylstannyl) stannane (146 mg, 1.1 equiv) in THF (2 mL) was added Pd(PPh₃)₄ (46.9 mg, 0.1 equiv) and LiCl (51.6 mg, 3.0 equiv). The mixture was stirred at 60° C. for 16 hours under nitrogen atmosphere. The mixture was filtered. The filtrate was diluted with EtOAc (30 mL) and quenched by KF aqueous solution (50 mL). The mixture was filtered and concentrated. The residue was purified by column chromatography (SiO₂, petroleum ether/ethyl acetate 100:1 to 10:1) to afford the title compound (50.0 mg, 47% yield, mixture) as a light yellow oil; ¹H NMR (400 MHZ, CHLOROFORM-d) δ=7.38-7.30 (m, 13H), 6.05-5.85 (m, 1H), 4.20-4.16 (m, 2H), 3.87 (t, J=5.6 Hz, 2H), 3.70-3.60 (m, 2H), 2.54-2.48 (m, 2H), 2.45-2.38 (m, 1H), 1.83 (td, J=5.6, 11.4 Hz, 2H), 0.14-0.09 (m, 8H)

Step C. 7-(8-ethylnaphthalen-1-yl)-8-fluoro-4-(methylthio)-2-((tetrahydro-1H-pyrrolizin-7a(5H)-yl)methoxy)pyrido[4,3-d]pyrimidine: To a solution of 7-(8-ethylnaphthalen-1-yl)-8-fluoro-2-((tetrahydro-1H-pyrrolizin-7a(5H)-yl)methoxy)-4-(2,2,2-trifluoroethoxy)pyrido[4,3-d]pyrimidine (1.00 g, 1.0 equiv) in DCM (20 mL) was added NaSMe (233 mg, 1.8 equiv). The mixture was stirred at 20° C. for 16 hours. The mixture was filtered and concentrated. The residue was purified by column chromatography (SiO₂, petroleum ether/ethyl acetate 10:1 to 0:1) to afford the title compound (650 mg, 72% yield) as a light yellow solid; ¹H NMR (400 MHZ, CHLOROFORM-d) δ=9.23-9.16 (m, 1H), 7.99 (dd, J=1.2, 8.1 Hz, 1H), 7.81 (d, J=8.2 Hz, 1H), 7.56-7.50 (m, 1H), 7.47 (t, J=7.6 Hz, 1H), 7.43 (dd, J=1.2, 7.2 Hz, 1H), 7.37 (d, J=7.2 Hz, 1H), 4.36 (s, 2H), 3.19-3.08 (m, 2H), 2.80 (s, 3H), 2.67 (td, J=6.8, 10.2 Hz, 3H), 2.45-2.25 (m, 3H), 2.09 (br dd, J=6.2, 12.5 Hz, 3H), 1.90 (quin, J=6.5 Hz, 5H), 1.70 (td, J=7.5, 12.6 Hz, 3H), 1.01-0.88 (m, 4H)

Step D. 7-(8-ethylnaphthalen-1-yl)-8-fluoro-2-((tetrahydro-1H-pyrrolizin-7a(5H)-yl)methoxy)-4-(2,3,6,7-tetrahydrooxepin-4-yl)pyrido[4,3-d]pyrimidine: To a solution of 7-(8-ethylnaphthalen-1-yl)-8-fluoro-4-(methylthio)-2-((tetrahydro-1H-pyrrolizin-7a(5H)-yl)methoxy)pyrido[4,3-d]pyrimidine (50.0 mg, 1.0 equiv) and trimethyl (2,3,6,7-tetrahydrooxepin-4-yl) stannane (48.1 mg, 1.8 equiv, mixture) in THF (2 mL) was added tris(2-furyl)phosphane (7.13 mg, 0.3 equiv), thiophene-2-carbonyloxycopper (29.3 mg, 1.5 equiv) and Pd₂(dpa)₃ (9.37 mg, 0.1 equiv). The mixture was stirred at 60° C. for 16 hours under nitrogen atmosphere. The mixture was diluted with water (20 mL) and extracted with EtOAc (10 mL×2). The combined organic layers were washed with brine (20 mL), dried over anhydrous sodium sulfate and concentrated. The residue was purified by column chromatography (SiO₂, petroleum ether/ethyl acetate 10:1 to 0:1) to afford the title compound (45.0 mg, 80.6% yield, 98.8% purity, mixture) as a light yellow oil; LCMS (ESI, M+1): m/z=539.1.

Step E. rac-(R)-7-(8-ethylnaphthalen-1-yl)-8-fluoro-4-(oxepan-4-yl)-2-((tetrahydro-1H-pyrrolizin-7a(5H)-yl)methoxy)pyrido[4,3-d]pyrimidine: To a solution of 7-(8-ethylnaphthalen-1-yl)-8-fluoro-2-((tetrahydro-1H-pyrrolizin-7a(5H)-yl)methoxy)-4-(2,3,6,7-tetrahydrooxepin-4-yl)pyrido[4,3-d]pyrimidine (35.0 mg, 1.0 equiv) in EtOAc (1.5 mL) was added Pd/C (30.0 mg, 10% purity, wet). The mixture was stirred at 20° C. for 3 hours under H₂ atmosphere (15 psi). The mixture was diluted with EtOAc (10 mL) and filtered. The filtrate was concentrated. The residue was purified by prep-HPLC [Phenomenex Synergi C18 150×25 mm×10 um; A: water (FA), B: ACN, B %: 26%-46% over 10 min] and lyophilized to afford the title compound (3.24 mg, 8.8% yield, 95.9% purity) as a white solid (0.45 formic acid salt); ¹H NMR (400 MHZ, CHLOROFORM-d) δ=9.32 (s, 1H), 8.36 (br s, 1H), 8.00 (d, J=8.3 Hz, 1H), 7.83 (d, J=8.0 Hz, 1H), 7.56-7.51 (m, 1H), 7.48 (t, J=7.6 Hz, 1H), 7.41 (dd, J=6.8, 15.3 Hz, 2H), 4.82-4.69 (m, 2H), 4.07-3.88 (m, 4H), 3.82-3.72 (m, 3H), 3.01-2.89 (m, 2H), 2.49-2.31 (m, 5H), 2.23 (br dd, J=6.6, 12.7 Hz, 4H), 2.17-2.05 (m, 3H), 2.04-1.90 (m, 4H), 0.97 (br t, J=7.6 Hz, 3H); LCMS (ESI, M+1): m/z=541.3.

### Example 724

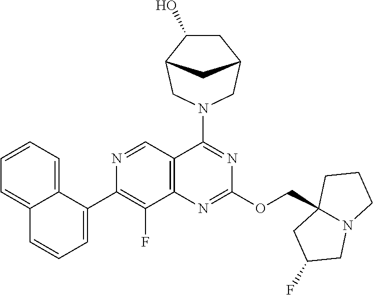 [SMILES: O[C@@H]1C[C@H]2C[C@@H]1CN(c1nc(OC[C@@]34CCCN3C[C@H](F)C4)nc3c(F)c(-c4cccc5ccccc45)ncc13)C2]

### (1R,5R,6R)-3-(8-fluoro-2-(((2R,7aS)-2-fluorohexahydro-1H-pyrrolizin-7a-yl)methoxy)-7-(naphthalen-1-yl)pyrido[4,3-d]pyrimidin-4-yl)-3-azabicyclo[3.2.1]octan-6-ol

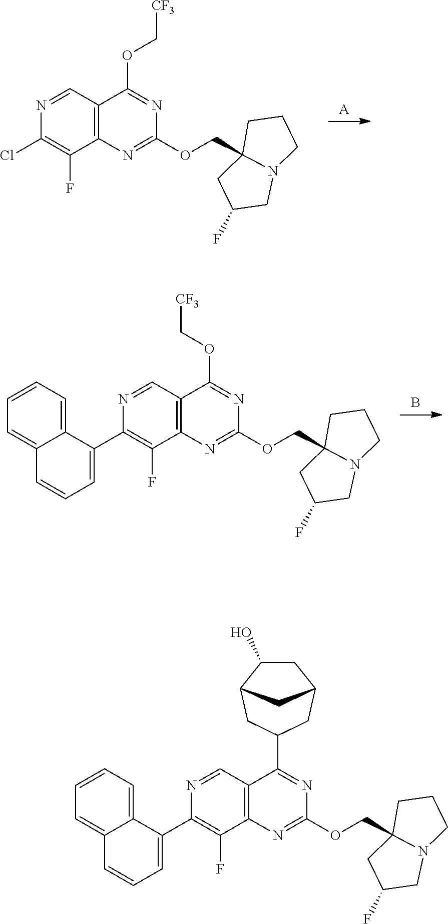

Step A. 8-fluoro-2-(((2R,7aS)-2-fluorohexahydro-1H-pyrrolizin-7a-yl)methoxy)-7-(naphthalen-1-yl)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (300 mg, 1.0 equiv) and Cs₂CO₃ (1.50 M, 1.37 mL, 3.0 equiv) in methoxycyclopentane (8 mL) were added AdanBuP-Pd-G3 (49.8 mg, 0.1 equiv) and 4,4,5,5-tetramethyl-2-(1-naphthyl)-1,3,2-dioxaborolane (261 mg, 1.5 equiv). The mixture was stirred at 100° C. for 3 hours. The reaction mixture was diluted with water (10 mL). The aqueous was extracted with EtOAc (3×10 mL). The combined organic layers were dried over anhydrous sodium sulfate, concentrated, and purified by reversed phase flash chromatography [C18, 0.1% formic acid condition] to afford the title compound (180 mg, 47% yield) as a yellow solid; LCMS (ESI, M+1): m/z=531.2.

Step B. (1R,5R,6R)-3-(8-fluoro-2-(((2R,7aS)-2-fluorohexahydro-1H-pyrrolizin-7a-yl)methoxy)-7-(naphthalen-1-yl)pyrido[4,3-d]pyrimidin-4-yl)-3-azabicyclo[3.2.1]octan-6-ol: To a solution of 8-fluoro-2-(((2R,7aS)-2-fluorohexahydro-1H-pyrrolizin-7a-yl)methoxy)-7-(naphthalen-1-yl)-4-(2,2,2-trifluoroethoxy)pyrido[4,3-d]pyrimidine (100 mg, 1.0 equiv) in DMF (0.3 mL) were added DIPEA (73.1 mg, 3.0 equiv) and (1R,5R,6R)-3-azabicyclo[3.2.1]octan-6-ol (36.0 mg, 1.5 equiv). The mixture was stirred at 40° C. for 12 hours. The reaction mixture was filtered, concentrated and purified by prep-HPLC [Phenomenex luna C18 Ultra 150×25 mm×10 μm; A: water (10 mM FA), B: ACN, B %: 15%-45% over 10 min] and lyophilized to afford the title compound (58.1 mg, 51% yield, 0.8FA) as a yellow solid; ¹H NMR (400 MHZ, METHANOL-d₄) δ=9.32 (s, 1H), 8.08-8.02 (m, 1H), 7.99 (d, J=8.4 Hz, 1H), 7.71-7.61 (m, 3H), 7.57-7.51 (m, 1H), 7.50-7.44 (m, 1H), 5.58-5.39 (m, 1H), 4.98-4.91 (m, 1H), 4.84-4.78 (m, 1H), 4.61-4.48 (m, 2H), 4.38-4.29 (m, 1H), 3.86-3.59 (m, 4H), 3.55-3.45 (m, 1H), 3.36-3.26 (m, 1H), 2.67-2.38 (m, 3H), 2.36-2.18 (m, 5H), 2.14-2.03 (m, 1H), 1.98-1.88 (m, 1H), 1.86-1.74 (m, 1H), 1.44-1.31 (m, 1H); LCMS (ESI, M+1): m/z=558.2.

### Example 725

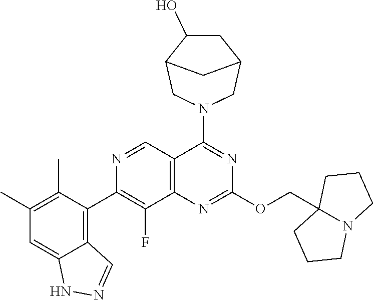 [SMILES: Cc1cc2[nH]ncc2c(-c2ncc3c(N4CC5CC(O)C(C5)C4)nc(OCC45CCCN4CCC5)nc3c2F)c1C]

### 3-(7-(5,6-dimethyl-1H-indazol-4-yl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol

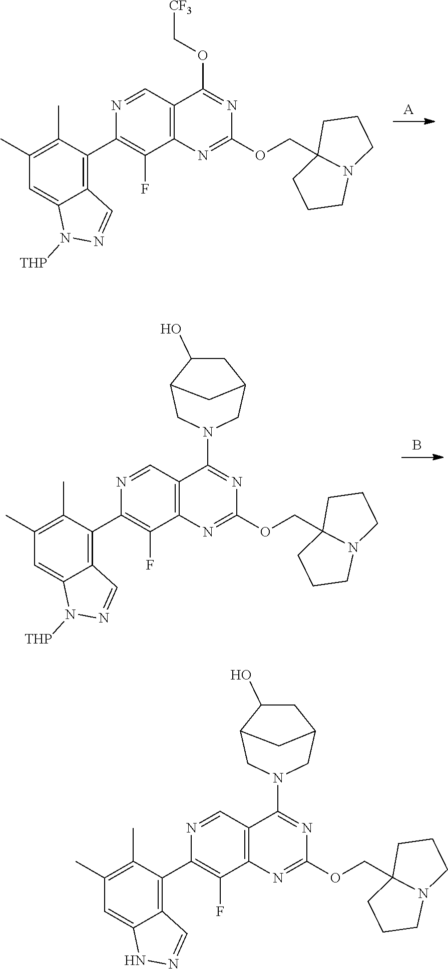

Step A. 3-(7-(5,6-dimethyl-1-(tetrahydro-2H-pyran-2-yl)-1H-indazol-4-yl)-8-fluoro-2-((hexahydro-1H-pyrrolizin-7a-yl)methoxy)pyrido[4,3-d]pyrimidin-4-yl)-3-azabicyclo[3.2.1]octan-6-ol: To a mixture of 7-(5,6-dimethyl-1-(tetrahydro-2H-pyran-2-yl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine (100 mg, 1.0 equiv), 3-azabicyclo[3.2.1]octan-6-ol (52 mg, 2.5 equiv) in DMF (0.6 mL) was added DIPEA (42.0 mg, 2.0 equiv) and 4 Å molecular sieves (10 mg). The mixture was stirred at 40° C. for 13 hours. The mixture was filtered, concentrated and purified with reversed phase flash chromatography [water (FA, 0.1%)/acetonitrile=3/1] to afford the tittle compound (97 mg, 90% yield) as a light yellow solid; LCMS (ESI, M+1): m/z=642.5.

Step B. 3-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-3-azabicyclo[3.2.1]octan-6-ol: To a solution of 3-(7-(5,6-dimethyl-1-(tetrahydro-2H-pyran-2-yl)-1H-indazol-4-yl)-8-fluoro-2-((hexahydro-1H-pyrrolizin-7a-yl)methoxy)pyrido[4,3-d]pyrimidin-4-yl)-3-azabicyclo[3.2.1]octan-6-ol (95 mg, 1.0 equiv) in DCM (1 mL) was added TFA (1.5 mL) dropwise at 0° C. The mixture was stirred at 25° C. for 1 hour. The mixture was poured into DCM (10 mL) and saturated NaHCO₃ aqueous (20 mL) at 0° C. The pH of the mixture was adjusted to 9 with solid Na₂CO₃ below 10° C. The mixture was extracted with DCM (4×5 mL). The combined organic layer was dried over anhydrous Na₂SO₄, concentrated and purified with prep-HPLC [column: Phenomenex Synergi C18 150×25 mm×10 um; mobile phase: water (FA)-ACN; B %: 9%-39%, 10 minutes] to afford the tittle compound (38.3 mg, 46% yield, 0.42FA) as a white solid; ¹H NMR (400 MHZ, methanol-d₄) δ=9.34 (br d, J=5.5 Hz, 1H), 7.59 (br d, J=9.2 Hz, 1H), 7.51 (s, 1H), 5.00-4.92 (m, 2H), 4.85-4.72 (m, 1H), 4.34 (br dd, J=4.8, 10.4 Hz, 1H), 3.80 (br dd, J=13.6, 16.0 Hz, 1H), 3.64-3.43 (m, 3H), 3.23-3.07 (m, 2H), 2.51 (s, 3H), 2.41 (br s, 1H), 2.36-1.88 (m, 15H), 1.87-1.77 (m, 1H), 1.42-1.32 (m, 1H); LCMS (ESI, M+1): m/z=558.4.

### Example 726

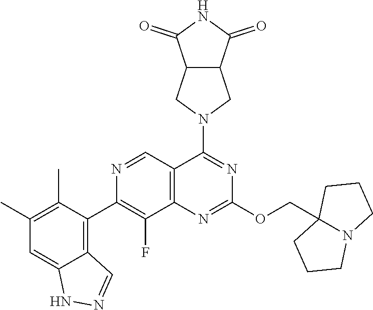 [SMILES: Cc1cc2[nH]ncc2c(-c2ncc3c(N4CC5C(=O)NC(=O)C5C4)nc(OCC45CCCN4CCC5)nc3c2F)c1C]

### 5-(7-(5,6-dimethyl-1H-indazol-4-yl)-8-fluoro-2-((hexahydro-1H-pyrrolizin-7a-yl)methoxy)pyrido[4,3-d]pyrimidin-4-yl)tetrahydropyrrolo[3,4-c]pyrrole-1,3(2H,3aH)-dione

The title compound was synthesized from Intermediate 19 according to the 2-step procedure described for example 725 to produce the desired compound as a white solid (0.52 formic acid salt); ¹H NMR (400 MHZ, methanol-d4) δ=9.37 (s, 1H), 7.60 (s, 1H), 7.52 (s, 1H), 4.71-4.61 (m, 2H), 4.44-4.29 (m, 2H), 3.84-3.69 (m, 2H), 3.58-3.47 (m, 2H), 3.18-3.08 (m, 2H), 2.51 (s, 3H), 2.32-1.95 (m, 13H); LCMS (ESI, M+1): m/z=571.4.

### Example 727

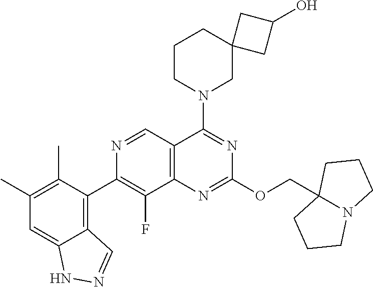 [SMILES: Cc1cc2[nH]ncc2c(-c2ncc3c(N4CCCC5(CC(O)C5)C4)nc(OCC45CCCN4CCC5)nc3c2F)c1C]

### 6-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-6-azaspiro[3.5]nonan-2-ol

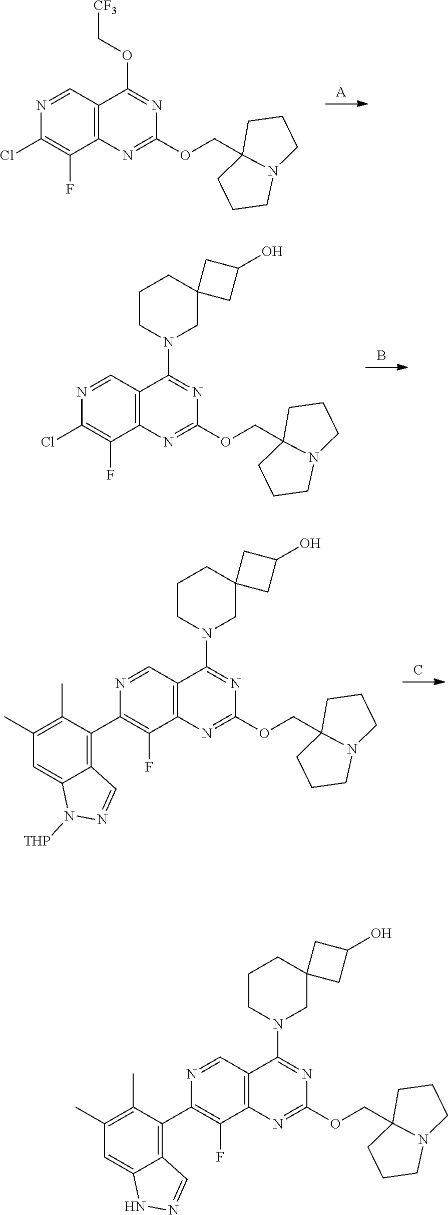

Step A. 6-(7-chloro-8-fluoro-2-((tetrahydro-1H-pyrrolizin-7a(5H)-yl)methoxy)pyrido[4,3-d]pyrimidin-4-yl)-6-azaspiro[3.5]nonan-2-ol: To a mixture of 7-chloro-8-fluoro-2-((hexahydro-1H-pyrrolizin-7a-yl)methoxy)-4-(2,2,2-trifluoroethoxy)pyrido[4,3-d]pyrimidine (0.9 g, 1.0 equiv), 6-azaspiro[3.5]nonan-2-ol (604 mg, 1.6 equiv, HCl) in DMF (5 mL) were added DIPEA (1.93 g, 7.0 equiv) and 4 Å molecular sieves (100 mg). The mixture was stirred at 40° C. for 18 hours under N₂ atmosphere. The mixture was filtered and purified by reversed phase flash chromatography [water (FA, 0.1%)/acetonitrile 4:1 to 3:1] to afford the title compound (380 mg, 38% yield) as a yellow oil.

Step B. 6-(7-(5,6-dimethyl-1-(tetrahydro-2H-pyran-2-yl)-1H-indazol-4-yl)-8-fluoro-2-((hexahydro-1H-pyrrolizin-7a-yl)methoxy)pyrido[4,3-d]pyrimidin-4-yl)-6-azaspiro[3.5]nonan-2-ol: To a solution of 6-(7-chloro-8-fluoro-2-((hexahydro-1H-pyrrolizin-7a-yl)methoxy)pyrido[4,3-d]pyrimidin-4-yl)-6-azaspiro[3.5]nonan-2-ol (135 mg, 1.0 equiv), 5,6-dimethyl-1-(tetrahydro-2H-pyran-2-yl)-4-(4,4,5,5-tetramethyl-1,3,2-dioxaborolan-2-yl)-1H-indazole (115 mg, 1.1 equiv), K₃PO₄ (1.5 M, 585 μL, 3.0 equiv) in CPME (2.5 mL) was added CataCXium A Pd G3 (213 mg, 1.0 equiv) under N₂. The mixture was stirred at 90° C. for 1.5 hours under N₂. The mixture was diluted with water (10 mL) and extracted with ethyl acetate (2×10 mL). The organic layer was washed with brine (10 mL) and dried over Na₂SO₄. The solvent was concentrated and purified by reversed phase flash chromatography [C18, 0.1% formic acid condition] to afford the title compound (30.0 mg, 16% yield) as a yellow oil; LCMS (ESI, M+1): m/z=656.5

Step C. 6-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-6-azaspiro[3.5]nonan-2-ol: To a solution of 6-(7-(5,6-dimethyl-1-(tetrahydro-2H-pyran-2-yl)-1H-indazol-4-yl)-8-fluoro-2-((hexahydro-1H-pyrrolizin-7a-yl)methoxy)pyrido[4,3-d]pyrimidin-4-yl)-6-azaspiro[3.5]nonan-2-ol (95.0 mg, 1.0 equiv) in DCM (1 mL) was added TFA (1.54 g, 93 equiv) at 20° C. The mixture was stirred at 20° C. for 1 hour. The mixture was poured into DCM (10 mL) and saturated NaHCO₃ aqueous (20 mL) at 0° C. The pH of the mixture was adjusted to 9 with solid Na₂CO₃ below 10° C. The mixture was extracted with DCM (2×10 mL). The organic layer was washed with brine (10 mL), dried over Na₂SO₄ and concentrated. The residue was purified by prep-HPLC [column: Unisil 3-100 C18 Ultra 150×50 mm×3 μm; A: water (FA), B: ACN, B %: 8%-38% over 7 minutes] and prep-HPLC [column: Waters Xbridge 150×25 mm×5 μm; A: water (10 mM NH₄HCO₃), B: ACN, B %: 27%-57% over 8 minutes] to afford the title compound (16.1 mg, 19% yield) as a yellow solid. ¹H NMR (400 MHZ, METHANOL-d₄) δ=9.19 (d, J=6.4 Hz, 1H), 7.62 (s, 1H), 7.52 (s, 1H), 4.68 (d, J=7.2 Hz, 1H), 4.58 (s, 1H), 4.74-4.55 (m, 1H), 4.31 (br d, J=8.3 Hz, 1H), 4.13 (s, 1H), 4.11-3.98 (m, 3H), 3.76-3.63 (m, 2H), 3.29-3.23 (m, 1H), 2.52 (s, 3H), 2.43-2.28 (m, 3H), 2.26-1.98 (m, 10H), 1.84 (br s, 6H); LCMS (ESI, M+1): m/z=572.4.

### Example 728

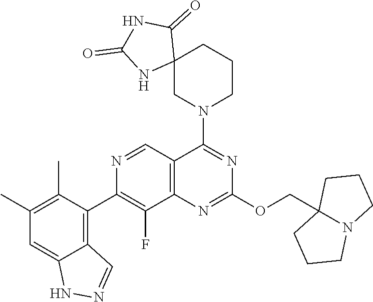 [SMILES: Cc1cc2[nH]ncc2c(-c2ncc3c(N4CCCC5(C4)NC(=O)NC5=O)nc(OCC45CCCN4CCC5)nc3c2F)c1C]

### 7-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione

The title compound was synthesized from Intermediate 19 according to the 2-step procedure described for example 725 to produce the desired compound as a white solid; white solid (0.54 formic acid salt); ¹H NMR (400 MHZ, METHANOL-d4) δ=9.22 (s, 1H), 7.60 (s, 1H), 7.52 (s, 1H), 4.65 (br d, J=13.2 Hz, 1H), 4.60-4.51 (m, 2H), 4.52-4.44 (m, 1H), 3.91-3.77 (m, 2H), 3.62-3.52 (m, 2H), 3.20-3.12 (m, 2H), 2.51 (s, 3H), 2.32-2.25 (m, 2H), 2.22 (s, 3H), 2.19-2.09 (m, 5H), 2.07-1.94 (m, 5H); LCMS (ESI, M+1): m/z=600.3.

### Example 729

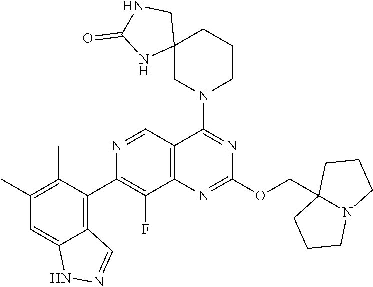 [SMILES: Cc1cc2[nH]ncc2c(-c2ncc3c(N4CCCC5(CNC(=O)N5)C4)nc(OCC45CCCN4CCC5)nc3c2F)c1C]

### 7-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decan-2-one

The title compound was synthesized from Intermediate 19 according to the 2-step procedure described for example 725 to produce the desired compound as a white solid (0.54 formic acid salt); ¹H NMR (400 MHZ, methanol-d4) δ=9.22-9.18 (m, 1H), 7.60 (s, 1H), 7.52 (s, 1H), 4.63-4.55 (m, 1H), 4.52-4.43 (m, 2H), 4.35-4.09 (m, 1H), 4.06-3.78 (m, 2H), 3.58-3.36 (m, 3H), 3.10-3.06 (m, 2H), 2.51 (s, 3H), 2.39-2.15 (m, 5H), 2.14-1.85 (m, 10H); LCMS (ESI, M+1): m/z=586.4.

### Example 730

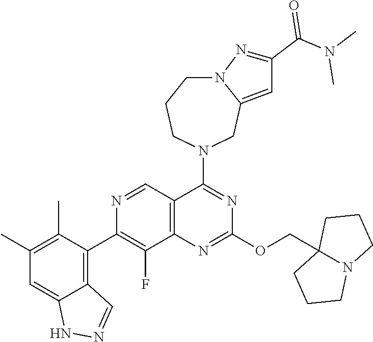 [SMILES: Cc1cc2[nH]ncc2c(-c2ncc3c(N4CCCn5nc(C(=O)N(C)C)cc5C4)nc(OCC45CCCN4CCC5)nc3c2F)c1C]

### 5-(7-(5,6-dimethyl-1H-indazol-4-yl)-8-fluoro-2-((hexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized according to the 3-step procedure described for example 727 to produce the desired compound as a yellow solid (0.21 formic acid salt); ¹H NMR (400 MHZ, METHANOL-d₄) δ=9.31 (s, 1H), 7.61 (s, 1H), 7.52 (s, 1H), 6.78 (s, 1H), 5.28 (s, 2H), 4.61 (s, 2H), 4.55-4.47 (m, 4H), 3.72-3.59 (m, 2H), 3.35 (s, 3H), 3.28-3.21 (m, 2H), 3.09 (s, 3H), 2.51 (s, 3H), 2.47 (br d, J=4.4 Hz, 2H), 2.36-2.28 (m, 2H), 2.22 (s, 4H), 2.20-2.13 (m, 3H), 2.12-2.04 (m, 2H); LCMS (ESI, M+1): m/z=639.5.

### Example 731

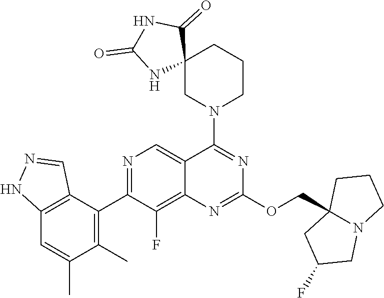 [SMILES: Cc1cc2[nH]ncc2c(-c2ncc3c(N4CCC[C@]5(C4)NC(=O)NC5=O)nc(OC[C@@]45CCCN4C[C@H](F)C5)nc3c2F)c1C]

### (5R)-7-(7-(5,6-dimethyl-1H-indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decane-2,4-dione

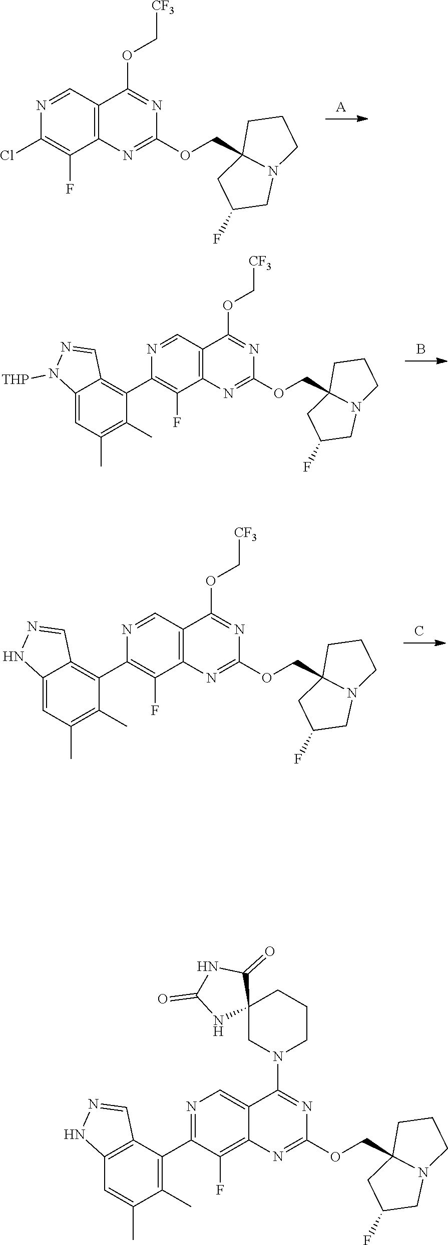

Step A: 7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (0.50 g, 1.14 mmol, 1.0 equiv) in CPME (6.0 mL) was added CataCXium A Pd G3 (83.0 mg, 0.1 equiv), 5,6-dimethyl-1-(tetrahydro-2H-pyran-2-yl)-4-(4,4,5,5-tetramethyl-1,3,2-dioxaborolan-2-yl)-1H-indazole (528 mg, 1.3 equiv) and Cs₂CO₃ (1.0 M, 3.0 mL, 2.6 equiv). The mixture was stirred at 80° C. for 4 hours. The mixture was quenched with H₂O (10 mL) and extracted with EtOAc (3×30 mL). The combined organic layer was washed with brine (3×50 mL), dried over Na₂SO₄, concentrated and purified by reversed phase flash chromatography [C18, 0.1% formic acid condition] to afford the title compound (370 mg, 51% yield) as a yellow solid; LCMS (ESI, M+1): 633.4.

Step B. 7-(5,6-dimethyl-1H-indazol-4-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a solution of 7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (250 mg, 1.0 equiv) in dichloromethane (1.0 mL) was added TFA (1.54 g, 1.0 mL). The mixture was stirred at 0˜15° C. for 16 hours. The mixture was quenched by addition of H₂O (10 mL) and extracted with ethyl acetate (3×30 mL). The combined organic layer was dried over Na₂SO₄ and concentrated to afford the title compound (110 mg, 50%) as a yellow solid; LCMS (ESI, M+1): 549.3.

Step C. (5R)-7-(7-(5,6-dimethyl-1H-indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decane-2,4-dione: To a solution of 7-(5,6-dimethyl-1H-indazol-4-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (110 mg, 1.0 equiv) in DMF (0.2 mL) was added DIPEA (77.8 mg, 3.0 equiv), 4 Å MS (20.0 mg) and (R)-1,3,7-triazaspiro[4.5]decane-2,4-dione (40.7 mg, 1.2 equiv). The mixture was stirred at 40° C. for 16 hours. The mixture was filtered. The filtrate was concentrated. The residue was purified by reversed phase flash chromatography [C18, 0.1% formic acid condition] to afford the title compound (51.4 mg, 39% yield) as a white solid (0.41 formic acid salt); column: Chiralcel OJ-3 50×4.6 mm I.D., 3 μm, mobile phase: 40% [MeOH+0.05% DEA] in CO₂, flow rate: 3 mL/min, detector: 220 nm, tR: 1.429 min; ¹H NMR (400 MHZ, Methanol-d4) δ 9.20 (d, J=1.6 Hz, 1H), 7.60 (s, 1H), 7.52 (s, 1H), 5.52-5.28 (m, 1H), 4.66 (br d, J=13.6 Hz, 1H), 4.54-4.39 (m, 3H), 3.81 (br d, J=13.6 Hz, 2H), 3.65-3.40 (m, 3H), 3.19 (br dd, J=4.0, 9.2 Hz, 1H), 2.51 (s, 3H), 2.49-2.19 (m, 7H), 2.17-1.92 (m, 6H); LCMS (ESI, M+1): m/z=618.4.

### Example 732

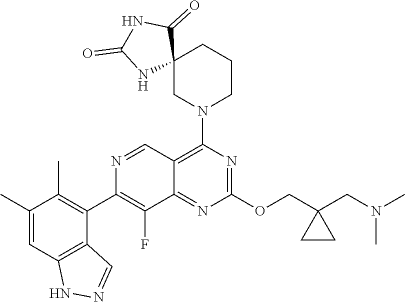

### (5R)-7-(7-(5,6-dimethyl-1H-indazol-4-yl)-2-((1-((dimethylamino)methyl)cyclopropyl)methoxy)-8-fluoropyrido[4,3-d]pyrimidin-4-yl)-1,3,7-triazaspiro[4.5]decane-2,4-dione

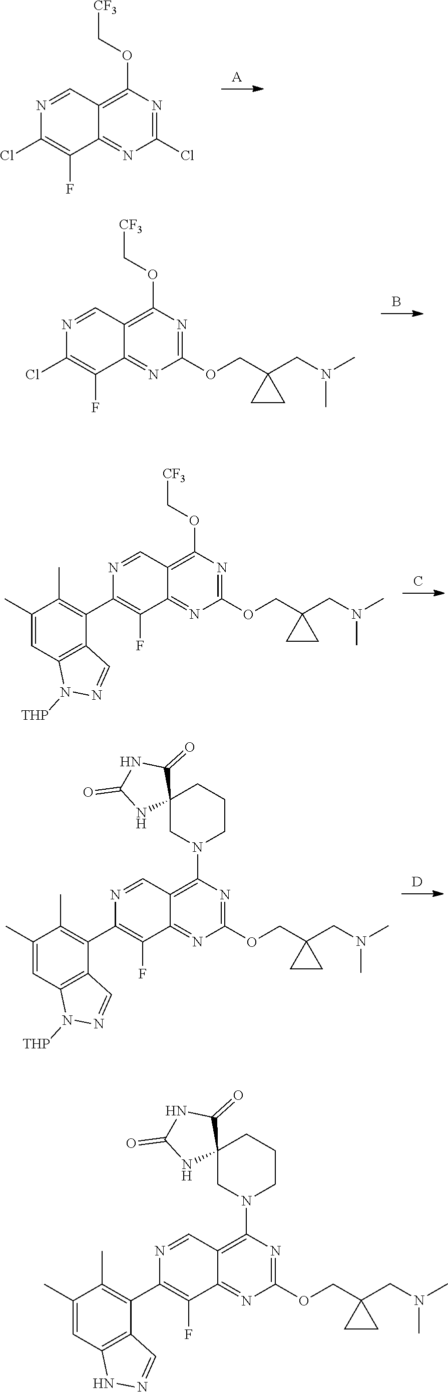

Step A. 1-(1-(((7-chloro-8-fluoro-4-(2,2,2-trifluoroethoxy)pyrido[4,3-d]pyrimidin-2-yl)oxy)methyl)cyclopropyl)-N,N-dimethylmethanamine: To a solution of 2,7-dichloro-8-fluoro-4-(2,2,2-trifluoroethoxy)pyrido[4,3-d]pyrimidine (5.0 g, 1.0 equiv), DIPEA (6.13 g, 3.0 equiv) and 4 Å molecular sieves (1.00 g) in THF (50 mL) was added (1-((dimethylamino)methyl)cyclopropyl)methanol (1.84 g, 0.9 equiv) at −40° C. The mixture was stirred at −40° C. for 0.5 hour. The mixture was diluted with water (50 mL) and extracted with EtOAc (3×50 mL). The combined organic layers were dried over anhydrous sodium sulfate, concentrated and purified by reversed phase flash chromatography [C18, 0.1% formic acid condition] to afford the title compound (2.20 g, 34% yield) as a yellow solid; LCMS (ESI, M+1): m/z=409.1.

Step B. 1-(1-(((7-(5,6-dimethyl-1-(tetrahydro-2H-pyran-2-yl)-1H-indazol-4-yl)-8-fluoro-4-(2,2,2-trifluoroethoxy)pyrido[4,3-d]pyrimidin-2-yl)oxy)methyl)cyclopropyl)-N,N-dimethylmethanamine: To a mixture of 1-(1-(((7-chloro-8-fluoro-4-(2,2,2-trifluoroethoxy)pyrido[4,3-d]pyrimidin-2-yl)oxy)methyl)cyclopropyl)-N,N-dimethylmethanamine (1.0 g, 1.0 equiv), 5,6-dimethyl-1-(tetrahydro-2H-pyran-2-yl)-4-(4,4,5,5-tetramethyl-1,3,2-dioxaborolan-2-yl)-1H-indazole (1.31 g, 1.5 equiv) and Cs₂CO₃ (1.5 M, 4.89 mL, 3.0 equiv) in methoxycyclopentane (10 mL) was added Ad₂nBuP-Pd G3 (178 mg, 0.1 equiv). The mixture was stirred at 90° C. for 3 hours under N₂. The mixture was diluted with water (10 mL) and extracted with EtOAc (3×10 mL). The combined organic layers were dried over anhydrous sodium sulfate, concentrated and purified by reversed phase flash chromatography [C18, 0.1% formic acid condition] to afford the title compound (950 mg, 63% yield) as a light-yellow solid; LCMS (ESI, M+1): m/z=603.3.

Step C. (5R)-7-(7-(5,6-dimethyl-1-(tetrahydro-2H-pyran-2-yl)-1H-indazol-4-yl)-2-((1-((dimethylamino)methyl)cyclopropyl)methoxy)-8-fluoropyrido[4,3-d]pyrimidin-4-yl)-1,3,7-triazaspiro[4.5]decane-2,4-dione: To a mixture of 1-(1-(((7-(5,6-dimethyl-1-(tetrahydro-2H-pyran-2-yl)-1H-indazol-4-yl)-8-fluoro-4-(2,2,2-trifluoroethoxy)pyrido[4,3-d]pyrimidin-2-yl)oxy)methyl)cyclopropyl)-N,N-dimethylmethanamine (150 mg, 1.0 equiv), (R)-1,3,7-triazaspiro[4.5]decane-2,4-dione (84.2 mg, 2.0 equiv) and 4 Å molecular sieves (20 mg) in DMF (1 mL) was added DIPEA (96.5 mg, 3.0 equiv). The mixture was stirred at 50° C. for 12 hours. The residue was filtered and washed with DMF (1 mL) and purified by reversed phase flash chromatography [C18, 0.1% formic acid condition] to afford the title compound (100 mg, 57% yield) as a yellow solid; LCMS (ESI, M/2+1): m/z=672.4.

Step D. (5R)-7-(7-(5,6-dimethyl-1H-indazol-4-yl)-2-((1-((dimethylamino)methyl)cyclopropyl)methoxy)-8-fluoropyrido[4,3-d]pyrimidin-4-yl)-1,3,7-triazaspiro[4.5]decane-2,4-dione: To a solution of (5R)-7-(7-(5,6-dimethyl-1-(tetrahydro-2H-pyran-2-yl)-1H-indazol-4-yl)-2-((1-((dimethylamino)methyl)cyclopropyl)methoxy)-8-fluoropyrido[4,3-d]pyrimidin-4-yl)-1,3,7-triazaspiro[4.5]decane-2,4-dione (50.0 mg, 1.0 equiv) in DCM (0.5 mL) was added TFA (2.31 g, 272 equiv) at 0° C. The mixture was stirred at 20° C. for 12 hours. The pH of the mixture was adjusted to 8 with saturated NaHCO₃ aqueous solution (5 mL) and the resulting was extracted with EtOAc (3×5 mL). The combined organic layers were dried over anhydrous sodium sulfate, concentrated and purified with prep-HPLC [Phenomenex C18 75×30 mm×3 μm; A: water (FA), B: ACN; B %: 8%-38% over 7 min] and lyophilized to afford the title compound (9.28 mg, 21% yield) as a white solid (0.40 formic acid salt); ¹H NMR (400 MHZ, METHANOL-d₄) δ=9.20 (d, J=1.2 Hz, 1H), 7.60 (s, 1H), 7.52 (s, 1H), 4.63 (br d, J=13.6 Hz, 1H), 4.54-4.34 (m, 3H), 3.88-3.73 (m, 2H), 2.96-2.77 (m, 2H), 2.63 (br s, 6H), 2.51 (s, 3H), 2.32-2.24 (m, 1H), 2.22 (s, 3H), 2.15-2.07 (m, 1H), 2.06-1.90 (m, 2H), 0.86 (s, 2H), 0.69 (br s, 2H); LCMS (ESI, M+1): m/z=588.5.

### Example 733

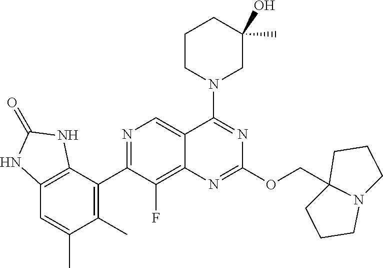 [SMILES: Cc1cc2[nH]c(=O)[nH]c2c(-c2ncc3c(N4CCC[C@@](C)(O)C4)nc(OCC45CCCN4CCC5)nc3c2F)c1C]

### 4-(8-fluoro-4-((R)-3-hydroxy-3-methylpiperidin-1-yl)-2-((tetrahydro-1H-pyrrolizin-7a(5H)-yl)methoxy)pyrido[4,3-d]pyrimidin-7-yl)-5,6-dimethyl-1,3-dihydro-2H-benzo[d]imidazol-2-one

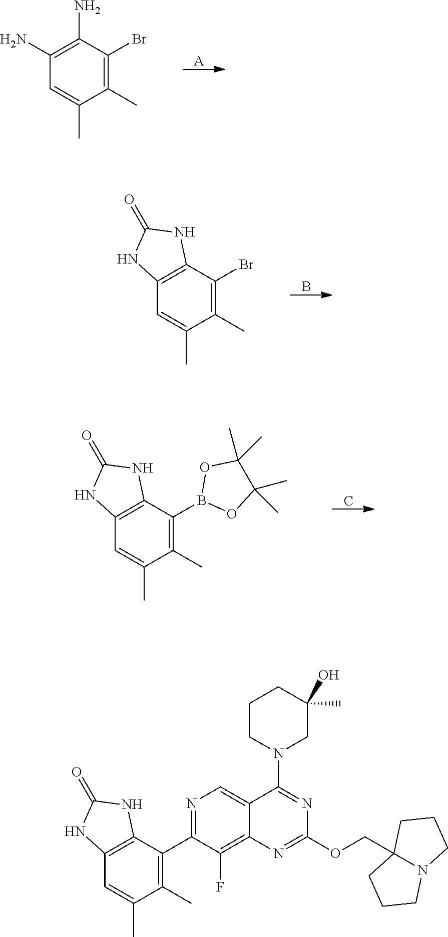

Step A. 4-bromo-5,6-dimethyl-1H-benzo[d]imidazol-2(3H)-one: To a solution of 3-bromo-4,5-dimethylbenzene-1,2-diamine (500 mg, 1.0 equiv) in tetrahydrofuran (10 mL) was added CDI (301 mg, 0.8 equiv). The mixture was stirred at 25° C. for 12 hours. The mixture was filtered. The filter cake was washed with H₂O (20 ml). The solid was dried under reduced pressure to afford the tittle compound (200 mg, 36% yield) as a a white solid; LCMS (ESI, M+1, M+3): m/z=241.0, 243.0.

Step B. 5,6-dimethyl-4-(4,4,5,5-tetramethyl-1,3,2-dioxaborolan-2-yl)-1H-benzo[d]imidazol-2(3H)-one: To a solution of 4-bromo-5,6-dimethyl-1H-benzo[d]imidazol-2(3H)-one (200 mg, 1.0 equiv) and 4,4,4′,4′,5,5,5′,5′-octamethyl-2,2′-bi (1,3,2-dioxaborolane) (527 mg, 2.5 equiv) in dioxane (5 mL) was added KOAc (244 mg, 3.0 equiv) and PCy₃ Pd G2 (49.0 mg, 0.1 equiv). The mixture was stirred at 80° C. for 16 hours. The mixture was quenched by addition of water (2 mL) and then extracted with ethyl acetate (3×5 mL). The combined organic layers were dried over anhydrous sodium sulfate, concentrated and purified by column chromatography (SiO₂, petroleum ether/ethyl acetate 5:1 to 1:1) to afford the title compound (110 mg, 41% yield) as a white solid; LCMS (ESI, M+1): m/z=289.2

Step C. 4-(8-fluoro-4-((R)-3-hydroxy-3-methylpiperidin-1-yl)-2-((tetrahydro-1H-pyrrolizin-7a(5H)-yl)methoxy)pyrido[4,3-d]pyrimidin-7-yl)-5,6-dimethyl-1,3-dihydro-2H-benzo[d]imidazol-2-one: To a solution of (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (50.0 mg, 1.0 equiv) and 5,6-dimethyl-4-(4,4,5,5-tetramethyl-1,3,2-dioxaborolan-2-yl)-1H-benzo[d]imidazol-2(3H)-one (39.6 mg, 1.2 equiv) in tetrahydrofuran (1.6 mL) and H₂O (0.4 mL) was added AdanBup-Pd-G3 (8.35 mg, 0.1 equiv) and K₃PO₄ (73.0 mg 3.0 equiv). The mixture was stirred at 60° C. for 12 hours. The mixture was diluted with water (2 mL) and then extracted with ethyl acetate (3×5 mL). The combined organic layers were dried over anhydrous sodium sulfate, concentrated and purified by prep-HPLC [column: Phenomenex luna C18 150×25 mm×10 μm; A: water (FA); B: ACN, B %: 10%-40%, over 10 min] and lyophilized to afford the title compound (5.38 mg, 9.0% yield) as a yellow solid (0.81 formic acid salt); SFC: Chiralpak IC-3 50×4.6 mm I.D., 3 μm, 40% 50% MeOH+ACN (0.05% DEA) in CO₂ Flow rate: 3 mL/min, detector: 220 nm, t_{R1}=0.900 min, t_{R2}=1.793 min; ¹H NMR (400 MHz, METHANOL-d4) δ=9.29 (br s, 1H), 8.54 (s, 1H), 8.96-8.40 (m, 1H), 7.02 (s, 1H), 4.68-4.52 (m, 3H), 4.40-4.32 (m, 1H), 3.68-3.52 (m, 3H), 3.48-3.36 (m, 1H), 3.28-3.08 (m, 2H), 2.37 (s, 3H), 2.32-2.24 (m, 2H), 2.24-2.12 (m, 4H), 2.12-2.08 (m, 3H), 2.08-1.96 (m, 3H), 1.91 (br s, 3H), 1.30 (s, 3H); LCMS (ESI, M+1): m/z=562.2.

### Example 734

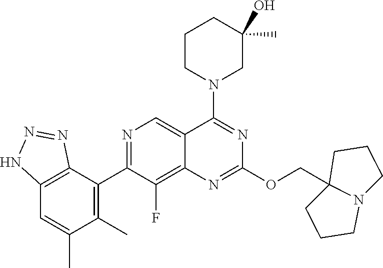 [SMILES: Cc1cc2[nH]nnc2c(-c2ncc3c(N4CCC[C@@](C)(O)C4)nc(OCC45CCCN4CCC5)nc3c2F)c1C]

### (3R)-1-(7-(5,6-dimethyl-1H-benzo[d][1,2,3]triazol-4-yl)-8-fluoro-2-((hexahydro-1H-pyrrolizin-7a-yl)methoxy)pyrido[4,3-d]pyrimidin-4-yl)-3-methylpiperidin-3-ol

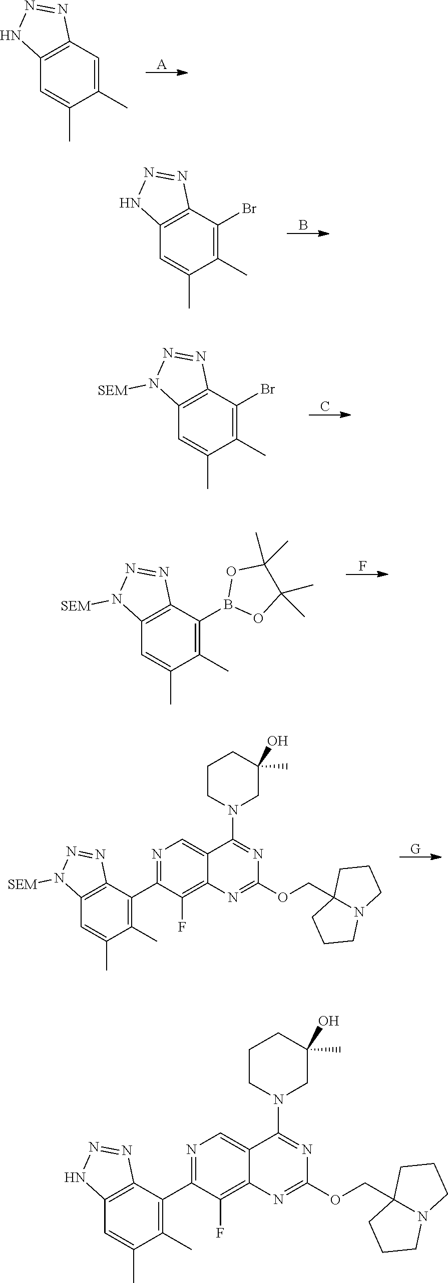

Step A. 4-bromo-5,6-dimethyl-1H-benzo[d][1,2,3]triazole: To a mixture of 5,6-dimethyl-1H-benzo[d][1,2,3]triazole (1.00 g, 1.0 equiv) in AcOH (10 mL) was added a solution of Br₂ (1.52 g, 1.4 equiv) in AcOH (10 mL) at 50° C. The mixture was stirred at 50° C. for 1 hour. The mixture was diluted with water (30 mL) and extracted with ethyl acetate (50 mL). The organic layer was dried over sodium sulfate and concentrated and purified by reversed-phase flash chromatography (C18, 0.1% FA condition) to afford the title compound (500 mg, 32% yield) as a white solid; LCMS (ESI, M+1, M+3): m/z=225.9, 227.9.

Step B. 4-bromo-5,6-dimethyl-1-((2-(trimethylsilyl)ethoxy)methyl)-1H-benzo[d][1,2,3]triazole: To a solution of 4-bromo-5,6-dimethyl-1H-benzo[d][1,2,3]triazole (300 mg, 1 equiv) in THF (9 mL) was added NaH (106 mg, 60% purity, 2.0 equiv) at 0° C. portionwise. The mixture was stirred at 0° C. for 0.5 hour. Then to the mixture was added SEM-Cl (332 mg, 1.5 equiv) at 0° C. The mixture was stirred at 0° C. for 1 hour and then quenched with water (20 mL) at 0° C. and extracted with ethyl acetate (30 mL). The organic layer was dried over sodium sulfate, concentrated to afford the title compound (245 mg, crude) as a yellow oil.

Step C. 5,6-dimethyl-4-(4,4,5,5-tetramethyl-1,3,2-dioxaborolan-2-yl)-1-((2-(trimethylsilyl)ethoxy)methyl)-1H-benzo[d][1,2,3]triazole; A mixture of 4-bromo-5,6-dimethyl-1-((2-(trimethylsilyl)ethoxy)methyl)-1H-benzo[d][1,2,3]triazole (130 mg, 1.0 equiv), BIS(PINACOLATO)DIBORON (232 mg, 2.5 equiv) and KOAc (130 mg, 3.6 equiv) in dioxane (3 mL) was degassed and purged with N₂ for 3 times. PCy₃ Pd G2 (21.5 mg, 0.1 equiv) was added and the mixture was stirred at 80° C. for 12 hours under N₂ atmosphere. The mixture was diluted with sat. aq. NaHCO₃ (12 mL) and extracted with ethyl acetate (20 mL). The organic layer was dried over sodium sulfate, concentrated to afford the title compound (257 mg, crude) as a white solid.

Step F. (3R)-1-(7-(5,6-dimethyl-1-((2-(trimethylsilyl)ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-3-methylpiperidin-3-ol: A mixture of (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (100 mg, 1.0 equiv), 5,6-dimethyl-4-(4,4,5,5-tetramethyl-1,3,2-dioxaborolan-2-yl)-1-((2-(trimethylsilyl)ethoxy)methyl)-1H-benzo[d][1,2,3]triazole (278 mg, 3.0 equiv), cataCXium A Pd G3 (16.7 mg, 0.1 equiv) and K₃PO₄ (1.5 M, 459 μL, 3.0 equiv) in methoxycyclopentane (10 mL) was degassed under vacuum and stirred at 90° C. for 2 hours. The mixture was diluted with H₂O (15 mL) and extracted with ethyl acetate (20 mL). The organic layer was dried over sodium sulfate, concentrated and purified by reversed-phase flash chromatography (C18, 0.1% FA condition) to afford the title compound (25 mg, 7.2% yield) as a yellow solid; LCMS (ESI, M+1): m/z=677.3.

Step G. (3R)-1-(7-(5,6-dimethyl-1H-benzo[d][1,2,3]triazol-4-yl)-8-fluoro-2-((hexahydro-1H-pyrrolizin-7a-yl)methoxy)pyrido[4,3-d]pyrimidin-4-yl)-3-methylpiperidin-3-ol: To a solution of (3R)-1-(7-(5,6-dimethyl-1-((2-(trimethylsilyl)ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-3-methylpiperidin-3-ol (23.0 mg, 1.0 equiv) in DCM (2 mL) was added TFA (1.54 g, 397 equiv) at 25° C. The mixture was stirred at 25° C. for 0.5 hour. The mixture was concentrated under vacuum. The residue was purified by prep-HPLC (Phenomenex Synergi C18 150×25 mm×10 um; mobile phase: A: water (FA), B: ACN; B %: 4% to 34% over 10 min) to afford the title compound (5.80 mg, 30% yield) as a yellow solid (0.80 formic acid salt); ¹H NMR (400 MHz, CHLOROFORM-d) δ=9.48-9.24 (m, 1H), 7.87 (s, 1H), 4.92-4.74 (m, 1H), 4.66 (br s, 2H), 4.34-4.19 (m, 1H), 3.85-3.68 (m, 2H), 3.12-2.81 (m, 4H), 2.66-2.49 (m, 1H), 2.42-2.31 (m, 4H), 2.30-2.07 (m, 5H), 2.05-1.73 (m, 7H), 1.71-1.58 (m, 1H), 1.30 (s, 3H); LCMS (ESI, M+1): m/z=547.4.

### Example 735

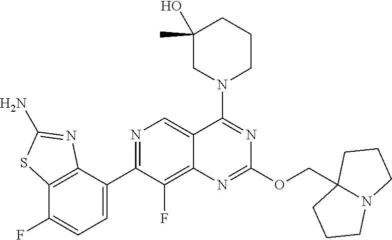 [SMILES: C[C@@]1(O)CCCN(c2nc(OCC34CCCN3CCC4)nc3c(F)c(-c4ccc(F)c5sc(N)nc45)ncc23)C1]

### (R)-1-(7-(2-amino-7-fluorobenzo[d]thiazol-4-yl)-8-fluoro-2-((hexahydro-1H-pyrrolizin-7a-yl)methoxy)pyrido[4,3-d]pyrimidin-4-yl)-3-methylpiperidin-3-ol

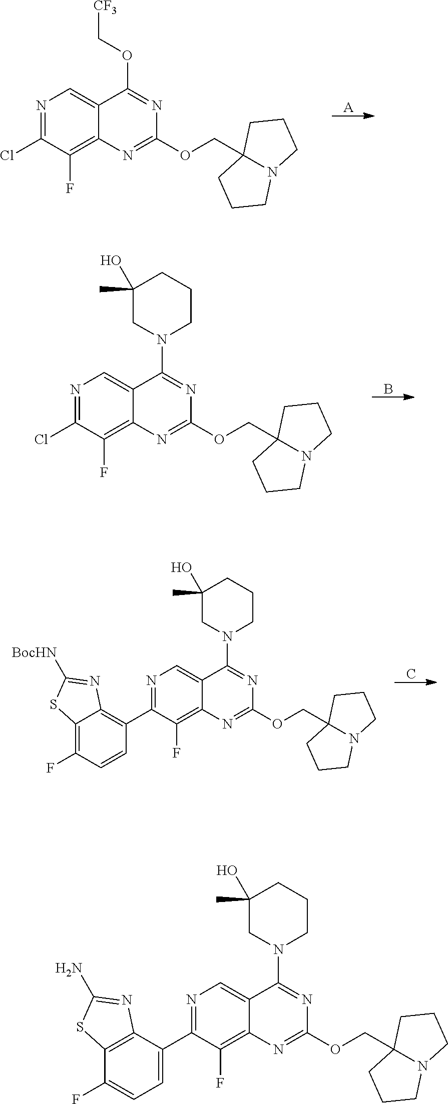

Step A. (R)-1-(7-chloro-8-fluoro-2-((hexahydro-1H-pyrrolizin-7a-yl)methoxy)pyrido[4,3-d]pyrimidin-4-yl)-3-methylpiperidin-3-ol: To a solution of (R)-3-methylpiperidin-3-ol hydrochloride (540 mg, 3.0 equiv) in DMF (3.0 mL) was added DIPEA (768 mg, 5.0 equiv) and 7-chloro-8-fluoro-2-((tetrahydro-1H-pyrrolizin-7a(5H)-yl)methoxy)-4-(2,2,2-trifluoroethoxy)pyrido[4,3-d]pyrimidine (500 mg, 1.0 equiv). The mixture was stirred at 40° C. for 16 hours. The mixture was diluted with water (20 mL) and then extracted with DCM (2×30 mL). The combined organic layers were washed with brine (30 mL), dried over anhydrous sodium sulfate, concentrated and purified by reversed phase flash chromatography (C18, 0.1% formic acid condition) to afford the title compound (177 mg, 92% yield) as a white solid. LCMS (ESI, M+1): m/z=436.2.

Step B. (R)-tert-butyl (7-fluoro-4-(8-fluoro-2-((hexahydro-1H-pyrrolizin-7a-yl)methoxy)-4-(3-hydroxy-3-methylpiperidin-1-yl)pyrido[4,3-d]pyrimidin-7-yl)benzo[d]thiazol-2-yl)carbamate: A mixture of (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (177 mg, 1.0 equiv), [2-(tert-butoxycarbonylamino)-7-fluoro-1,3-benzothiazol-4-yl]boronic acid (152 mg, 1.2 equiv), K₃PO₄ (1.5 M, 3.0 equiv) and Xphos Pd G4(34.9 mg, 0.1 equiv) in DMAc (4 mL) was degassed and stirred at 60° C. for 48 hours under N₂ atmosphere. The mixture was diluted with water (20 mL) and then extracted with DCM (2×30 mL). The combined organic layers were washed with brine (30 mL), dried over anhydrous sodium sulfate, concentrated and purified by reversed phase flash chromatography (C18, 0.1% formic acid condition) to afford the title compound (73.0 mg, 26% yield) as a white solid; LCMS (ESI, M+1): m/z=668.4

Step C. (R)-1-(7-(2-amino-7-fluorobenzo[d]thiazol-4-yl)-8-fluoro-2-((hexahydro-1H-pyrrolizin-7a-yl)methoxy)pyrido[4,3-d]pyrimidin-4-yl)-3-methylpiperidin-3-ol: The mixture of (R)-tert-butyl (7-fluoro-4-(8-fluoro-2-((hexahydro-1H-pyrrolizin-7a-yl)methoxy)-4-(3-hydroxy-3-methylpiperidin-1-yl)pyrido[4,3-d]pyrimidin-7-yl)benzo[d]thiazol-2-yl)carbamate (68.0 mg, 1.0 equiv) in HCl·MeOH (4 M, 6 mL) was stirred at 25° C. for 2.5 hours. The reaction mixture was concentrated and purified by prep-HPLC [column: Unisil 3-100 C18 Ultra 150×50 mm×3 μm; A: water (FA), B: ACN, B %: 13%-43% over 7 min] to afford the title compound (13.0 mg, 22% yield) as a yellow solid (0.75 formic acid salt). SFC analysis: Chiralcel OD-3 50×4.6 mm I.D., 3 μm Mobile phase: Phase A for CO₂, and Phase B for MeOH+ACN (0.05% DEA); Gradient elution: 40% MeOH+ACN (0.05% DEA) in CO₂. Flow rate: 3 mL/min; Detector: PDA; Column Temp: 35C; Back Pressure: 100 Bar”. ¹H NMR (400 MHZ, METHANOL-d₄) δ=9.23 (s, 1H), 7.52-7.44 (m, 1H), 7.09-6.95 (m, 1H), 4.65-4.52 (m, 3H), 4.30 (br d, J=13.2 Hz, 1H), 3.65-3.55 (m, 3H), 3.41 (br t, J=11.2 Hz, 1H), 3.25-3.16 (m, 2H), 2.33-2.24 (m, 2H), 2.23-2.08 (m, 5H), 2.07-1.97 (m, 2H), 1.91-1.82 (m, 1H), 1.82-1.70 (m, 2H), 1.28 (s, 3H); LCMS (ESI, M+1): m/z=568.3.

### Example 736

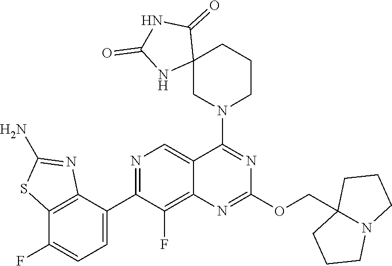 [SMILES: Nc1nc2c(-c3ncc4c(N5CCCC6(C5)NC(=O)NC6=O)nc(OCC56CCCN5CCC6)nc4c3F)ccc(F)c2s1]

### 7-(7-(2-amino-7-fluorobenzo[d]thiazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione

The title compound was synthesized from 7-chloro-8-fluoro-2-((tetrahydro-1H-pyrrolizin-7a(5H)-yl)methoxy)-4-(2,2,2-trifluoroethoxy)pyrido[4,3-d]pyrimidine according to the 3-step procedure described for example 735 to produce the desired compound as a white solid; ¹H NMR (400 MHZ, methanoL-d4) δ=9.14 (s, 1H), 7.49 (dd, J=5.6, 8.4 Hz, 1H), 7.03 (t, J=8.8 Hz, 1H), 4.70-4.59 (m, 3H), 4.50-4.39 (m, 1H), 3.98-3.61 (m, 4H), 3.36-3.32 (m, 1H), 3.29-3.21 (m, 1H), 2.32-1.99 (m, 12H); LCMS (ESI, M+1): m/z=622.3.

### Example 737

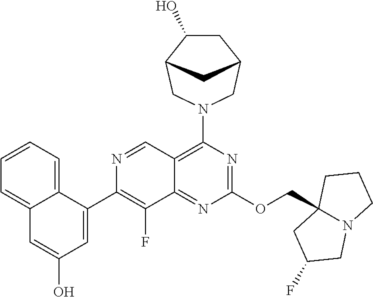 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1]

### (1R,5R,6R)-3-(8-fluoro-2-(((2R,7aS)-2-fluorohexahydro-1H-pyrrolizin-7a-yl)methoxy)-7-(3-hydroxynaphthalen-1-yl)pyrido[4,3-d]pyrimidin-4-yl)-3-azabicyclo[3.2.1]octan-6-ol

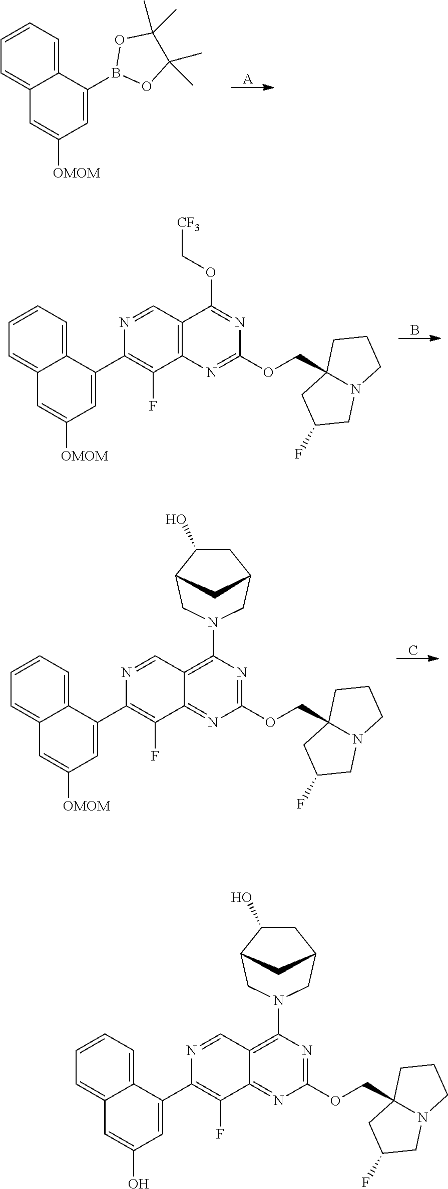

Step A. 8-fluoro-2-(((2R,7aS)-2-fluorohexahydro-1H-pyrrolizin-7a-yl)methoxy)-7-(3-(methoxymethoxy)naphthalen-1-yl)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (1.0 g, 1.0 equiv) and 2-[3-(methoxymethoxy)-1-naphthyl]-4,4,5,5-tetramethyl-1,3,2-dioxaborolane (859 mg, 1.2 equiv) in methoxycyclopentane (50 mL) was added Ad₂nBuP-Pd-G3 (166 mg, 0.1 equiv) and Cs₂CO₃ (1.5 M, 3.0 equiv). Then the mixture was stirred at 100° C. for 6 hours under N₂. The reaction mixture was diluted with H₂O (25 mL) and extracted with EtOAc (3×25 mL). The combined organic layers were washed with brine (20 mL), dried over Na₂SO₄, filtered and concentrated and purified by prep-HPLC [C18, 0.1% NH₃·H₂O condition] to afford the title compound (540 mg, 38% yield) as a yellow solid; ¹H NMR (400 MHZ, DMSO-d₆) δ=9.27 (s, 1H), 7.95 (d, J=8.0 Hz, 1H), 7.65 (d, J=2.0 Hz, 1H), 7.60-7.48 (m, 2H), 7.42 (d, J=2.0 Hz, 1H), 7.39-7.32 (m, 1H), 5.52-5.11 (m, 5H), 4.35-4.12 (m, 2H), 3.45 (s, 3H), 3.17-3.00 (m, 3H), 2.92-2.78 (m, 1H), 2.22-2.00 (m, 3H), 1.93-1.74 (m, 3H); LCMS (ESI, M+1): m/z=591.2.

Step B. (1R,5R,6R)-3-(8-fluoro-2-(((2R,7aS)-2-fluorotetrahydro-1H-pyrrolizin-7a(5H)-yl)methoxy)-7-(3-(methoxymethoxy)naphthalen-1-yl)pyrido[4,3-d]pyrimidin-4-yl)-3-azabicyclo[3.2.1]octan-6-ol: To a solution of 8-fluoro-2-(((2R,7aS)-2-fluorohexahydro-1H-pyrrolizin-7a-yl)methoxy)-7-(3-(methoxymethoxy)naphthalen-1-yl)-4-(2,2,2-trifluoroethoxy)pyrido[4,3-d]pyrimidine (200 mg, 1.0 equiv) and (1R,5R,6R)-3-azabicyclo[3.2.1]octan-6-ol (83.1 mg, 1.5 equiv, HCl) in DMF (0.6 mL) was added DIPEA (219 mg, 5.0 equiv) and 4 Å molecular sieves (80 mg). Then the mixture was stirred at 60° C. for 12 hours. The mixture was filtered and concentrated under reduced pressure. The crude product was purified with prep-HPLC [C18, 0.1% formic acid condition] to afford the title compound (130 mg, 57% yield) as a yellow solid; LCMS (ESI, M+1): m/z=618.3.

Step C. (1R,5R,6R)-3-(8-fluoro-2-(((2R,7aS)-2-fluorohexahydro-1H-pyrrolizin-7a-yl)methoxy)-7-(3-hydroxynaphthalen-1-yl)pyrido[4,3-d]pyrimidin-4-yl)-3-azabicyclo[3.2.1]octan-6-ol: A solution of (1R,5R,6R)-3-(8-fluoro-2-(((2R,7aS)-2-fluorotetrahydro-1H-pyrrolizin-7a(5H)-yl)methoxy)-7-(3-(methoxymethoxy)naphthalen-1-yl)pyrido[4,3-d]pyrimidin-4-yl)-3-azabicyclo[3.2.1]octan-6-ol (20.0 mg, 1.0 equiv) in HCl·MeOH (4 M, 1 mL, 123 equiv) was stirred at 0° C. for 2 hours. The mixture was concentrated under reduced pressure. Then the residue was dissolved in MeCN (5 mL). The pH was adjusted to 7 with solid NaHCO₃. The mixture was filtered and concentrated under reduced pressure. The crude product was purified with prep-HPLC [Phenomenex Synergi C18 150×25 mm×10 μm; A: water (FA), B: ACN; B %: 10%-40% over 10 min] to afford the title compound (7.0 mg, 37% yield, 0.33FA) as a white solid; ¹H NMR (400 MHZ, DMSO-d₆) δ=9.39 (s, 1H), 8.18 (s, 1H), 7.80 (d, J=8.4 Hz, 1H), 7.52 (br d, J=8.0 Hz, 1H), 7.44 (t, J=7.2 Hz, 1H), 7.32-7.19 (m, 3H), 5.42-5.17 (m, 1H), 4.87-4.51 (m, 3H), 4.25-4.09 (m, 2H), 4.03 (dd, J=4.0, 10.4 Hz, 1H), 3.72 (br d, J=12.4 Hz, 1H), 3.40-3.35 (m, 1H), 3.35 (br d, J=12.0 Hz, 1H), 3.13-3.05 (m, 2H), 3.02 (s, 1H), 2.90-2.75 (m, 1H), 2.33 (br d, J=1.6 Hz, 1H), 2.19-2.11 (m, 2H), 2.10-1.97 (m, 3H), 1.89-1.81 (m, 1H), 1.77 (br d, J=9.2 Hz, 3H), 1.70-1.60 (m, 1H), 1.26 (br d, J=14.4 Hz, 1H); LCMS (ESI, M+1): m/z=574.3.

### Example 738

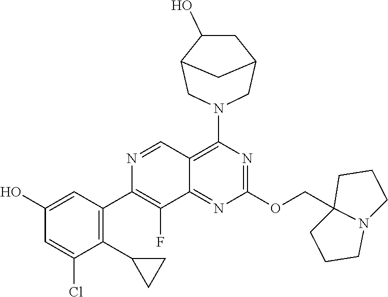 [SMILES: Oc1cc(Cl)c(C2CC2)c(-c2ncc3c(N4CC5CC(O)C(C5)C4)nc(OCC45CCCN4CCC5)nc3c2F)c1]

### (1S,5S,6R)-3-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol

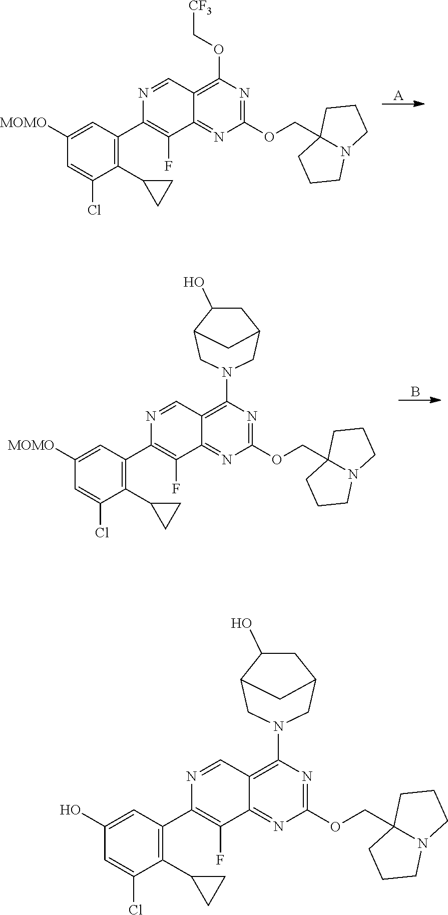

Step A. 3-(7-(3-chloro-2-cyclopropyl-5-(methoxymethoxy)phenyl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol: To a solution of 7-(3-chloro-2-cyclopropyl-5-(methoxymethoxy)phenyl)-8-fluoro-2-((tetrahydro-1H-pyrrolizin-7a(5H)-yl)methoxy)-4-(2,2,2-trifluoroethoxy)pyrido[4,3-d]pyrimidine (100 mg, 1.0 equiv) in DMF (1 mL) and ACN (1 mL) were added 3-azabicyclo[3.2.1]octan-6-ol (25.6 mg, 1.2 equiv) and K₃PO₄ (107 mg, 3.0 equiv). The reaction was stirred at 40° C. for 16 hours under nitrogen atmosphere. The reaction mixture was poured into saturated NH₄Cl aqueous solution (5 mL) and extracted with EtOAc (2 mL×3). The combined organic layers were washed with brine (5 mL), dried over anhydrous sodium sulfate and concentrated to afford the title compound (100 mg, crude) as a yellow oil; LCMS (ESI, M+1): m/z=624.5.

Step B. (1S,5S,6R)-3-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol: To a solution of 3-(7-(3-chloro-2-cyclopropyl-5-(methoxymethoxy)phenyl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol (80.0 mg, 1.0 equiv) in ACN (1 mL) was added HCl/dioxane (4 M, 3 mL, 93.6 equiv). The mixture was stirred at 25° C. for 0.5 hour. The reaction mixture was filtered, concentrated and purified with prep-HPLC [Phenomenex luna C18 150×25 mm×10 um; A: water (FA), B: ACN, B %: 13%-43% over 10 min] to afford the title compound (18.5 mg, 22% yield, (0.7 formic acid salt)) as a white solid; ¹H NMR (400 MHZ, METHANOL-d₄) δ=9.19 (s, 1H), 8.47 (s, 1H), 6.91 (d, J=2.8 Hz, 1H), 6.73 (d, J=2.4 Hz, 1H), 4.73 (d, J=12.4 Hz, 1H), 4.49 (s, 2H), 4.29-4.21 (m, 1H), 3.71 (d, J=12.0 Hz, 1H), 3.56-3.48 (m, 2H), 3.43 (d, J=12.8 Hz, 1H), 3.15-3.07 (m, 2H), 2.37-2.32 (m, 1H), 2.28-2.16 (m, 4H), 2.16-2.00 (m, 5H), 2.00-1.92 (m, 2H), 1.90-1.85 (m, 1H), 1.80-1.73 (m, 2H), 1.33-1.25 (m, 1H), 0.55 (s, 2H), 0.00 (s, 2H); LCMS (ESI, M+1): m/z=580.3.

### Example 739

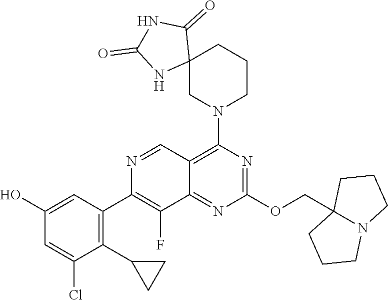 [SMILES: O=C1NC(=O)C2(CCCN(c3nc(OCC45CCCN4CCC5)nc4c(F)c(-c5cc(O)cc(Cl)c5C5CC5)ncc34)C2)N1]

### 7-(7-(3-chloro-2-cyclopropyl-5-hydroxyphenyl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione

The title compound was synthesized according to the 2-step procedure described for example 738 to produce the desired compound as a yellow solid; ¹H NMR (400 MHZ, METHANOL-d₄) δ=9.15 (s, 1H), 6.98 (d, J=2.6 Hz, 1H), 6.82-6.80 (m, 1H), 4.61-4.57 (m, 3H), 4.48-4.41 (m, 1H), 3.89-3.81 (m, 2H), 3.68-3.62 (m, 2H), 3.27-3.20 (m, 2H), 2.34-2.26 (m, 3H), 2.18 (dt, J=6.1, 12.8 Hz, 5H), 2.10-2.06 (m, 2H), 2.02-1.92 (m, 2H), 1.88-1.81 (m, 1H), 0.67-0.56 (m, 2H), 0.07 (br s, 2H); LCMS (ESI, M+1): m/z=622.3.

### Example 740

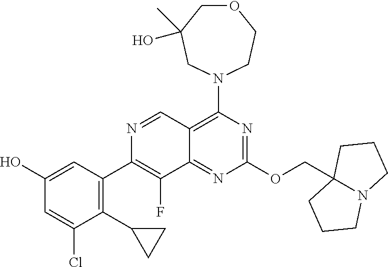 [SMILES: CC1(O)COCCN(c2nc(OCC34CCCN3CCC4)nc3c(F)c(-c4cc(O)cc(Cl)c4C4CC4)ncc23)C1]

### rac-(R)-4-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-6-methyl-1,4-oxazepan-6-ol

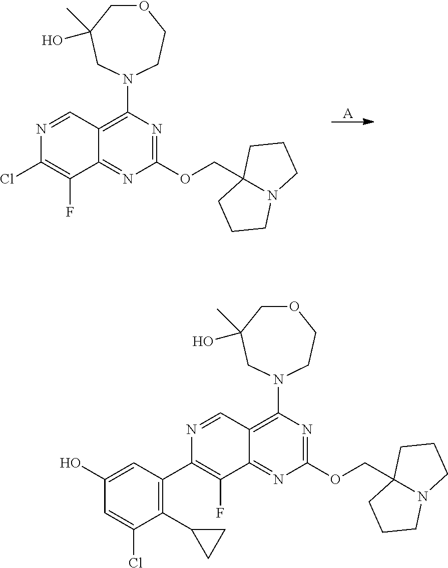

To a solution of 4-(7-chloro-8-fluoro-2-((tetrahydro-1H-pyrrolizin-7a(5H)-yl)methoxy)pyrido[4,3-d]pyrimidin-4-yl)-6-methyl-1,4-oxazepan-6-ol (30.0 mg, 1.0 equiv) and 3-chloro-4-cyclopropyl-5-(4,4,5,5-tetramethyl-1,3,2-dioxaborolan-2-yl) phenol (29.3 mg, 1.5 equiv) in CPME (0.8 mL) were added Ad₂nBuP-Pd-G₃ (9.67 mg, 0.2 equiv) and K₃PO₄ (1.5 M, 0.13 mL, 3.0 equiv). The mixture was stirred at 80° C. for 2 hours under nitrogen atmosphere. The reaction mixture was poured into saturated aqueous NH₄Cl solution (5 mL) and extracted with EtOAc (2 mL×3). The combined organic layers were washed with brine (5 mL), dried over anhydrous sodium sulfate, concentrated and purified with prep-HPLC [Phenomenex Luna C18 150×25 mm×10 um; A: water (FA), B: ACN, B %: 12%-42% over 10 min]. The product was dissolved in saturated aqueous NaHCO₃ solution (2 mL) and extracted with DCM (2 mL×3). The combined organic layers were washed with brine (2 mL), dried over anhydrous sodium sulfate and concentrated to afford the title compound (8.56 mg, 21% yield) as a yellow solid; ¹H NMR (400 MHz, METHANOL-d₄) δ=9.52-9.49 (m, 1H), 6.90 (d, J=2.6 Hz, 1H), 6.73 (d, J=2.4 Hz, 1H), 4.53-4.46 (m, 4H), 4.15-4.09 (m, 1H), 3.98-3.93 (m, 1H), 3.90-3.79 (m, 2H), 3.67-3.58 (m, 2H), 3.52-3.46 (m, 2H), 3.15-3.07 (m, 2H), 2.23-2.17 (m, 2H), 2.12-2.02 (m, 4H), 1.98-1.93 (m, 2H), 1.82-1.75 (m, 1H), 1.21 (s, 3H), 0.55 (br s, 2H), 0.01 (br dd, J=7.1, 10.6 Hz, 2H); LCMS (ESI, M+1): m/z=584.4.

### Example 741

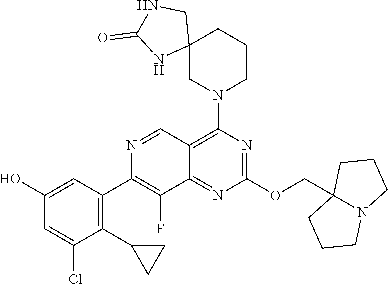 [SMILES: O=C1NCC2(CCCN(c3nc(OCC45CCCN4CCC5)nc4c(F)c(-c5cc(O)cc(Cl)c5C5CC5)ncc34)C2)N1]

### 7-(7-(3-chloro-2-cyclopropyl-5-hydroxyphenyl)-8-fluoro-2-((hexahydro-1H-pyrrolizin-7a-yl)methoxy)pyrido[4,3-d]pyrimidin-4-yl)-1,3,7-triazaspiro[4.5]decan-2-one

The title compound was synthesized according to the 2-step procedure described for example 738 to produce the desired compound as a as yellow solid (0.85 formic acid salt); ¹H NMR (400 MHZ, METHANOL-d₄) δ=9.14 (s, 1H), 8.54 (s, 1H), 6.98 (d, J=2.6 Hz, 1H), 6.81 (d, J=2.5 Hz, 1H), 4.65-4.56 (m, 2H), 4.55-4.48 (m, 1H), 4.35 (br d, J=12.8 Hz, 1H), 4.21 (br dd, J=4.1, 14.3 Hz, 1H), 4.03-3.94 (m, 1H), 3.89 (d, J=13.0 Hz, 1H), 3.63-3.51 (m, 2H), 3.42 (d, J=9.4 Hz, 1H), 3.23-3.11 (m, 2H), 2.33-2.22 (m, 2H), 2.14 (qd, J=6.9, 13.6 Hz, 4H), 2.07-1.94 (m, 5H), 1.93-1.80 (m, 2H), 0.76-0.52 (m, 2H), 0.07 (br d, J=1.5 Hz, 2H); LCMS (ESI, M+1): m/z=608.1.

### Example 742

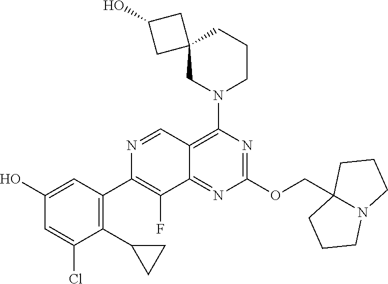 [SMILES: Oc1cc(Cl)c(C2CC2)c(-c2ncc3c(N4CCC[C@]5(C4)C[C@H](O)C5)nc(OCC45CCCN4CCC5)nc3c2F)c1]

### (2s,4r)-6-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-6-azaspiro[3.5]nonan-2-ol

### Example 743

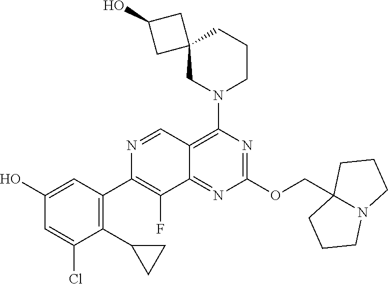 [SMILES: Oc1cc(Cl)c(C2CC2)c(-c2ncc3c(N4CCC[C@]5(C4)C[C@@H](O)C5)nc(OCC45CCCN4CCC5)nc3c2F)c1]

### (2r,4s)-6-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-6-azaspiro[3.5]nonan-2-ol

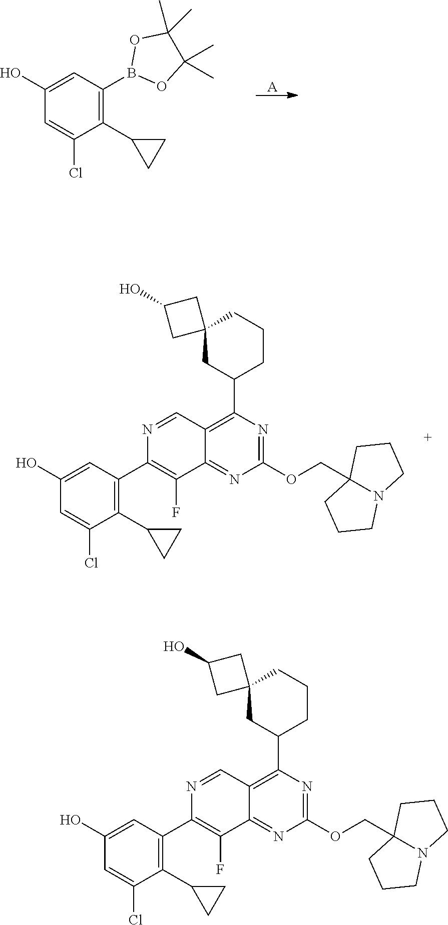

Step A. (2s,4r)-6-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-6-azaspiro[3.5]nonan-2-ol and (2r,4s)-6-(7-(3-chloro-2-cyclopropyl-5-hydroxyphenyl)-8-fluoro-2-((tetrahydro-1H-pyrrolizin-7a(5H)-yl)methoxy)pyrido[4,3-d]pyrimidin-4-yl)-6-azaspiro[3.5]nonan-2-ol: To a solution of 6-(7-chloro-8-fluoro-2-((tetrahydro-1H-pyrrolizin-7a(5H)-yl)methoxy)pyrido[4,3-d]pyrimidin-4-yl)-6-azaspiro[3.5]nonan-2-ol (40.0 mg, 1.0 equiv) and 3-chloro-4-cyclopropyl-5-(4,4,5,5-tetramethyl-1,3,2-dioxaborolan-2-yl) phenol (33.2 mg, 1.3 equiv) in CMPE (0.8 mL) were added Catacium Pd G3 (12.6 mg, 0.2 equiv) and K₃PO₄ (1.5 M, 0.17 mL, 3.0 equiv). The reaction was stirred at 80° C. for 2 hours under nitrogen atmosphere. The reaction mixture was poured into saturated NH₄Cl aqueous solution (5 mL) and extracted with EtOAc (2 mL×3). The combined organic layers were washed with brine (5 mL), dried over anhydrous sodium sulfate, concentrated and purified by prep-HPLC [Phenomenex Luna C18 150×25 mm×10 um; A: water (FA), B: ACN, B %: 13%-43% over 10 min] to afford the title compound EXAMPLE 742 (3.22 mg, 5.5% yield, 0.4FA) as a yellow solid and EXAMPLE 743 (7.44 mg, 13% yield, 0.4FA) as a yellow solid;

EXAMPLE 742: ¹H NMR (400 MHZ, METHANOL-d₄) δ=9.10 (s, 1H), 8.52 (s, 1H), 6.98 (d, J=2.4 Hz, 1H), 6.81 (d, J=2.6 Hz, 1H), 4.63 (s, 2H), 4.32-4.24 (m, 1H), 4.04 (s, 4H), 3.65 (dd, J=6.8, 11.6 Hz, 2H), 3.26-3.23 (m, 2H), 2.31 (br dd, J=6.7, 12.0 Hz, 4H), 2.24 (br s, 4H), 2.12-2.05 (m, 3H), 1.82 (s, 4H), 1.73-1.69 (m, 2H), 0.63 (d, J=6.4 Hz, 2H), 0.07 (br s, 2H); LCMS (ESI, M+1): m/z=594.5.

EXAMPLE 743: ¹H NMR (400 MHZ, METHANOL-d₄) δ=9.05-9.00 (m, 1H), 8.46-8.43 (m, 1H), 6.91 (d, J=2.4 Hz, 1H), 6.74 (d, J=2.4 Hz, 1H), 4.60-4.57 (m, 2H), 4.24-4.19 (m, 1H), 4.05-3.92 (m, 4H), 3.63-3.56 (m, 2H), 3.21-3.15 (m, 2H), 2.25 (dd, J=6.4, 12.4 Hz, 2H), 2.17-2.08 (m, 6H), 2.06-1.96 (m, 3H), 1.77-1.74 (m, 4H), 1.73-1.59 (m, 2H), 0.56 (d, J=6.4 Hz, 2H), 0.05-−0.05 (m, 2H); LCMS (ESI, M+1): m/z=594.3.

### Example 744

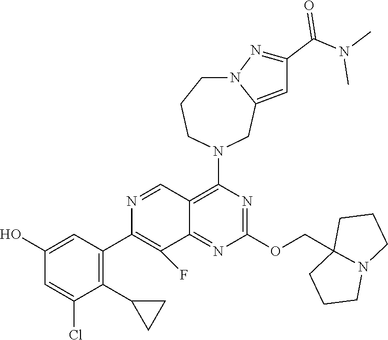 [SMILES: CN(C)C(=O)c1cc2n(n1)CCCN(c1nc(OCC34CCCN3CCC4)nc3c(F)c(-c4cc(O)cc(Cl)c4C4CC4)ncc13)C2]

### 5-(7-(3-chloro-2-cyclopropyl-5-hydroxyphenyl)-8-fluoro-2-((hexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from Intermediate 23 according to the procedure described for example 740 to produce the desired compound as white solid (0.64FA); ¹H NMR (400 MHZ, methanol-d4) δ=9.21 (s, 1H), 6.97 (d, J=2.4 Hz, 1H), 6.80 (d, J=2.4 Hz, 1H), 6.76 (s, 1H), 5.26 (s, 2H), 4.55-4.52 (m, 4H), 4.47 (br t, J=5.2 Hz, 2H), 3.54-3.51 (m, 2H), 3.34 (s, 3H), 3.14-3.11 (m, 2H), 3.10-3.03 (m, 3H), 2.44 (br d, J=3.2 Hz, 2H), 2.24 (dd, J=6.4, 12.4 Hz, 2H), 2.19-2.06 (m, 4H), 2.05-1.95 (m, 2H), 1.84 (dt, J=2.0, 5.6 Hz, 1H), 0.61 (br d, J=6.4 Hz, 2H), 0.06 (br d, J=1.6 Hz, 2H); LCMS (ESI, M+1): m/z=661.3.

### Example 745

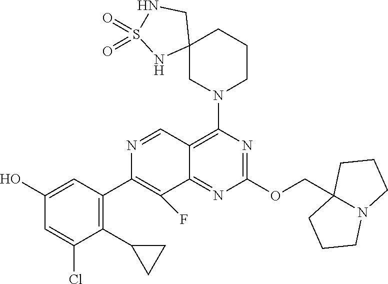 [SMILES: O=S1(=O)NCC2(CCCN(c3nc(OCC45CCCN4CCC5)nc4c(F)c(-c5cc(O)cc(Cl)c5C5CC5)ncc34)C2)N1]

### 7-(7-(3-chloro-2-cyclopropyl-5-hydroxyphenyl)-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide

The title compound was synthesized according to the 2-step procedure described for example 738 to produce the desired compound as a white solid; ¹H NMR (400 MHZ, METHANOL-d₄) δ=9.08 (s, 1H), 6.95 (d, J=2.6 Hz, 1H), 6.79 (d, J=2.6 Hz, 1H), 4.56 (br d, J=13.5 Hz, 1H), 4.40 (br d, J=13.5 Hz, 1H), 4.37-4.26 (m, 2H), 3.81 (d, J=13.3 Hz, 1H), 3.72-3.62 (m, 1H), 3.41 (d, J=11.9 Hz, 1H), 3.22-3.17 (m, 1H), 3.17-3.09 (m, 2H), 2.80-2.70 (m, 2H), 2.16-2.01 (m, 4H), 2.00-1.83 (m, 7H), 1.82-1.72 (m, 2H), 0.64 (br s, 2H), 0.08 (br s, 2H); LCMS (ESI, M+1): m/z=644.3.

### Example 746

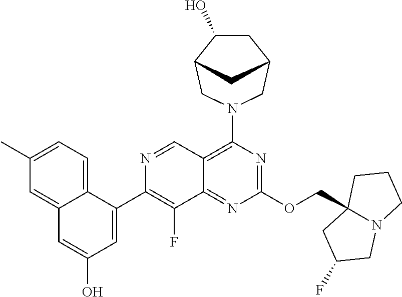 [SMILES: Cc1ccc2c(-c3ncc4c(N5C[C@@H]6C[C@H](C5)[C@H](O)C6)nc(OC[C@@]56CCCN5C[C@H](F)C6)nc4c3F)cc(O)cc2c1]

### (1R,5R,6R)-3-(8-fluoro-2-(((2R,7aS)-2-fluorotetrahydro-1H-pyrrolizin-7a(5H)-yl)methoxy)-7-(3-hydroxy-6-methylnaphthalen-1-yl)pyrido[4,3-d]pyrimidin-4-yl)-3-azabicyclo[3.2.1]octan-6-ol

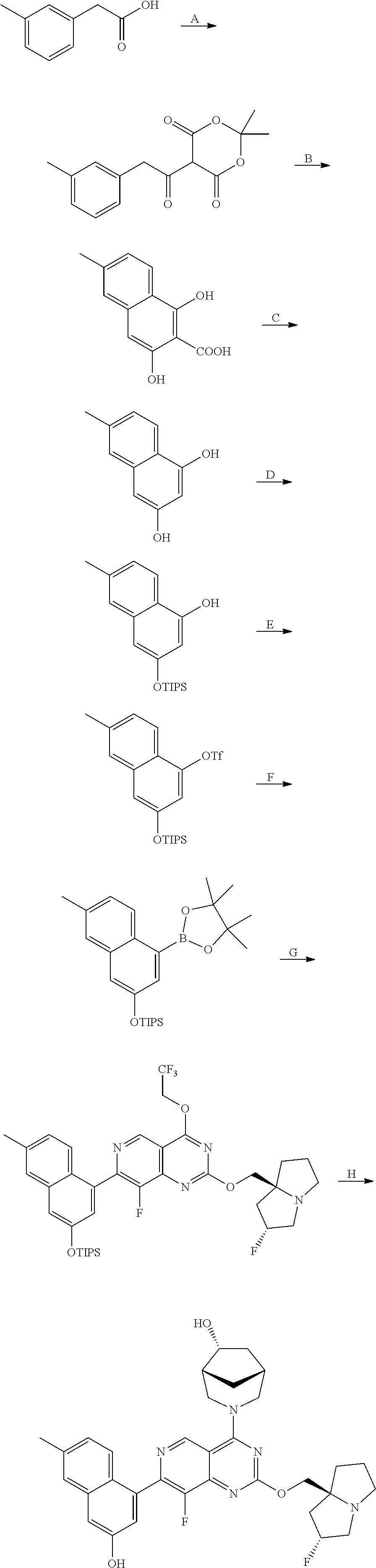

Step A. 2,2-dimethyl-5-(2-(m-tolyl)acetyl)-1,3-dioxane-4,6-dione: To a solution of 2-(m-tolyl) acetic acid (8.60 g, 1.0 equiv) and 2,2-dimethyl-1,3-dioxane-4,6-dione (9.90 g, 1.2 equiv) in DCM (90.0 mL) was added DMAP (8.40 g, 1.2 equiv) and EDCI (13.2 g, 1.2 equiv) at 0° C. The mixture was stirred at 25° C. for 12 hours. The reaction mixture was washed with water (100 mL) and the organic layer was dried over Na₂SO₄, concentrated and purified by column chromatography [SiO₂, Petroleum ether/Ethyl acetate 10:1 to 3:1] to afford the title compound (12.0 g, 72% yield) as a brown oil; ¹H NMR (400 MHZ, DMSO-d₆) δ=7.24-7.18 (m, 1H), 7.14-7.04 (m, 3H), 4.29-4.27 (m, 2H), 2.28 (s, 3H), 1.72-1.68 (s, 6H).

Step B. 1,3-dihydroxy-6-methyl-2-naphthoic acid: 2,2-dimethyl-5-[2-(m-tolyl) acetyl]-1,3-dioxane-4,6-dione (10.0 g, 1.0 equiv) was added into CF₃SO₃H (80.0 mL), then the mixture was stirred at 0° C. for 2 hours. (200 mL) ice water was added to the reaction mixture at 0° C., and then filtered. The filter cake was washed with water (150 mL) and dried to afford the title compound (12.0 g, crude) as a yellow solid; LCMS (ESI, M+1): m/z=219.1.

Step C. 6-methylnaphthalene-1,3-diol: 1,3-dihydroxy-6-methyl-naphthalene-2-carboxylic acid (10.0 g, 1.0 equiv) was dissolved in ACN (60.0 mL) and H₂O (60.0 mL), then stirred at 85° C. for 12 hours. The reaction mixture was extracted with ethyl acetate (3×100 mL). The combined organic layers were washed with brine (50 mL), dried over anhydrous sodium sulfate, concentrated and purified by reversed phase flash chromatography [C18, 0.1% formic acid condition] to afford the title compound (3.30 g, 39% yield) as a yellow solid; ¹H NMR (400 MHz, DMSO-d₆) δ=9.95 (s, 1H), 9.37 (s, 1H), 7.83 (d, J=8.5 Hz, 1H), 7.31 (s, 1H), 6.98 (d, J=8.4 Hz, 1H), 6.48 (s, 1H), 6.42 (s, 1H), 2.37 (s, 3H); LCMS (ESI, M+1): m/z=175.1.

Step D. 6-methyl-3-((triisopropylsilyl)oxy)naphthalen-1-ol: To a solution of 6-methylnaphthalene-1,3-diol (3.00 g, 1.0 equiv) in DCM (30.0 mL) was added DIPEA (4.45 g, 2.0 equiv) and TIPSCl (2.99 g. 0.9 equiv). The mixture was stirred at 0° C. for 0.5 hour. The reaction mixture was diluted with water (50 mL) and extracted with ethyl acetate (3×50 mL). The combined organic layers were washed with brine (50 mL), dried over anhydrous sodium sulfate, concentrated and purified by column chromatography [SiO₂, Petroleum ether/Ethyl acetate 50:1 to 10:1] to afford the title compound (1.40 g, crude) as a brown oil; LCMS (ESI, M+1): m/z=331.3.

Step E. 6-methyl-3-((triisopropylsilyl)oxy)naphthalen-1-yl trifluoromethanesulfonate: To a solution of 6-methyl-3-triisopropylsilyloxy-naphthalen-1-ol (1.30 g, 1.0 equiv) in DCM (15.0 mL) was added DIPEA (1.52 g, 3.0 equiv) and Tf₂O (1.66 g, 1.5 equiv). The mixture was stirred at −40° C. for 0.5 hour. The reaction mixture was filtered, concentrated and purified by column chromatography [SiO₂, Petroleum ether] to afford the title compound (1.40 g, 75% yield) as a colorless oil; LCMS (ESI, M+1): m/z=463.3.

Step F triisopropyl((7-methyl-4-(4,4,5,5-tetramethyl-1,3,2-dioxaborolan-2-yl)naphthalen-2-yl)oxy) silane: To a solution of (6-methyl-3-triisopropylsilyloxy-1-naphthyl)trifluoromethanesulfonate (1.39 g, 1.0 equiv), Bis(pinacolato)diboron (1.14 g, 1.5 equiv) in dioxane (20.0 mL) was added Pd(dppf)Cl₂ (220 mg, 0.1 equiv) and KOAc (885 mg, 3.0 equiv). The mixture was stirred at 100° C. for 6 hours. The reaction mixture was filtered, concentrated and purified by column chromatography [SiO₂, Petroleum ether/Ethyl acetate 100:1 to 50:1] to afford the title compound (1.15 g, 83% yield) as a yellow oil; LCMS (ESI, M+1): m/z=441.4.

Step G. 8-fluoro-2-(((2R,7aS)-2-fluorotetrahydro-1H-pyrrolizin-7a(5H)-yl)methoxy)-7-(6-methyl-3-((triisopropylsilyl)oxy)naphthalen-1-yl)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (900 mg, 1.0 equiv), triisopropyl-[[7-methyl-4-(4,4,5,5-tetramethyl-1,3,2-dioxaborolan-2-yl)-2-naphthyl]oxy]silane (1.08 g, 1.2 equiv) in methoxycyclopentane (20.0 mL) was added AdanBup-Pd-G3 (149 mg, 0.1 equiv) and Cs₂CO₃ (1.5 M, 3.00 equiv). The mixture was stirred at 100° C. for 6 hours. The reaction mixture was diluted with water (50 mL) and extracted with ethyl acetate (3×50 mL). The combined organic layers were washed with brine (50 mL), dried over anhydrous sodium sulfate, concentrated and purified by column chromatography [SiO₂, Petroleum ether/Ethyl acetate=10:1 to 1:1] to afford the title compound (500 mg, crude) as a yellow solid; LCMS (ESI, M+1): m/z=717.6.

Step H. (1R,5R,6R)-3-(8-fluoro-2-(((2R,7aS)-2-fluorotetrahydro-1H-pyrrolizin-7a(5H)-yl)methoxy)-7-(3-hydroxy-6-methylnaphthalen-1-yl)pyrido[4,3-d]pyrimidin-4-yl)-3-azabicyclo[3.2.1]octan-6-ol: To a solution of 8-fluoro-2-(((2R,7aS)-2-fluorotetrahydro-1H-pyrrolizin-7a(5H)-yl)methoxy)-7-(6-methyl-3-((triisopropylsilyl)oxy)naphthalen-1-yl)-4-(2,2,2-trifluoroethoxy)pyrido[4,3-d]pyrimidine (10.0 mg, 1.0 equiv) in DMF (0.5 mL) was added DIPEA (14.4 mg, 8.0 equiv), (1R,5R,6R)-3-azabicyclo[3.2.1]octan-6-ol (1 equiv) and 4 Å molecular sieves (10.0 mg). The mixture was stirred at 60° C. for 12 hours. The reaction mixture was filtered, and concentrated purified by prep-HPLC [Phenomenex Synergi C18 150×25 mm×10 μm; A: water (FA), B: ACN; B %: 13%-43% over 10 min] to afford the title compound (5.00 mg, 59% yield) as a yellow solid (0.30 formic acid salt); ¹H NMR (400 MHZ, DMSO-d6) δ=9.94-9.89 (m, 1H), 9.38 (s, 1H), 7.57 (s, 1H), 7.42 (br d, J=6.4 Hz, 1H), 7.19 (d, J=2.4 Hz, 1H), 7.13 (d, J=2.0 Hz, 1H), 7.09 (dd, J=1.6, 8.8 Hz, 1H), 5.38-5.17 (m, 1H), 4.78-4.57 (m, 2H), 4.12 (dd, J=7.2, 10.5 Hz, 1H), 4.06-3.98 (m, 1H), 3.77-3.68 (m, 1H), 3.13-3.05 (m, 2H), 3.01 (s, 1H), 2.88-2.78 (m, 1H), 2.70-2.63 (m, 3H), 2.42 (s, 3H), 2.37-2.29 (m, 3H), 2.19-2.10 (m, 2H), 2.06-1.98 (m, 2H), 1.87-1.80 (m, 1H), 1.77 (br d, J=9.6 Hz, 2H), 1.71-1.60 (m, 1H), 1.31-1.21 (m, 1H); LCMS (ESI, M+1): m/z=588.4.

### Example 747

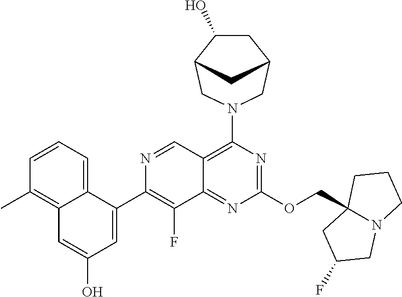 [SMILES: Cc1cccc2c(-c3ncc4c(N5C[C@@H]6C[C@H](C5)[C@H](O)C6)nc(OC[C@@]56CCCN5C[C@H](F)C6)nc4c3F)cc(O)cc12]

### (1R,5R,6R)-3-(8-fluoro-2-(((2R,7aS)-2-fluorohexahydro-1H-pyrrolizin-7a-yl)methoxy)-7-(3-hydroxy-5-methylnaphthalen-1-yl)pyrido[4,3-d]pyrimidin-4-yl)-3-azabicyclo[3.2.1]octan-6-ol

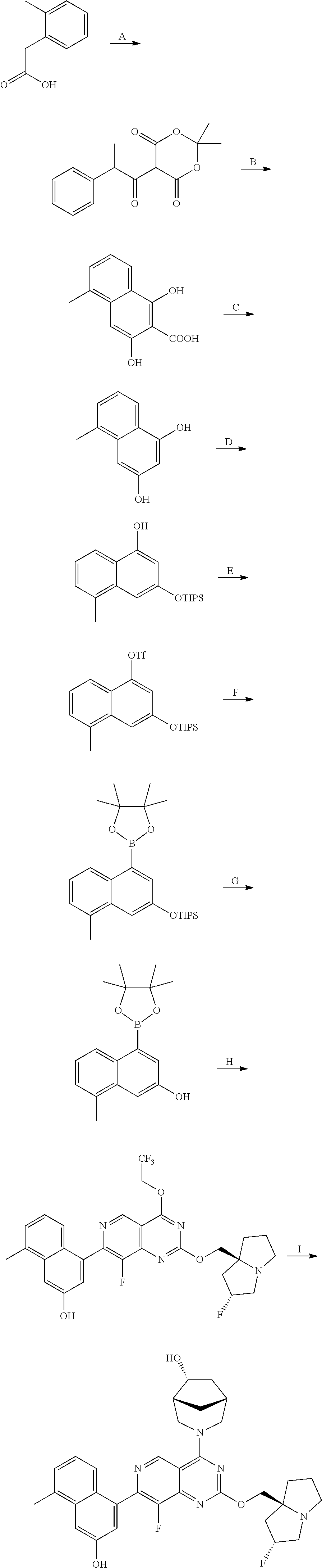

Steps A-F. triisopropyl((8-methyl-4-(4,4,5,5-tetramethyl-1,3,2-dioxaborolan-2-yl)naphthalen-2-yl)oxy) silane: The title compound was synthesized from 2-(o-tolyl) acetic acid according to the 6-step procedure described for example 746 Steps A-F to produce the desired compound (3.5 g) as a light yellow oil; ¹H NMR (400 MHZ, DMSO-d₆) δ=8.40 (d, J=7.6 Hz, 1H), 7.57 (d, J=2.8 Hz, 1H), 7.43 (d, J=2.4 Hz, 1H), 7.34-7.28 (m, 2H), 2.56 (s, 3H), 1.36 (s, 12H), 1.34-1.28 (m, 3H), 1.11 (d, J=7.6 Hz, 18H).

Step G. 8-methyl-4-(4,4,5,5-tetramethyl-1,3,2-dioxaborolan-2-yl)naphthalen-2-ol: To a solution of triisopropyl((8-methyl-4-(4,4,5,5-tetramethyl-1,3,2-dioxaborolan-2-yl)naphthalen-2-yl)oxy) silane (2.0 g, 1.0 equiv) in DMF (10 mL) was added CsF (6.90 g, 10 equiv) and the resulting was stirred for 15 mins. The mixture was poured into H₂O (100 mL) and ethyl acetate (30 mL) maintaining the temperature below 5° C. The mixture was extracted with ethyl acetate (3×10 mL). The combined organic layer was dried over anhydrous Na₂SO₄, concentrated and purified with column chromatography [SiO₂, petroleum ether/ethyl acetate 10:1 to 5:1] to afford the tittle compound (0.95 g, 63% yield, 86% purity) as a brown solid; LCMS (ESI, M+1): m/z=285.1.

Step H. 4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-8-methylnaphthalen-2-ol: To a mixture of 8-methyl-4-(4,4,5,5-tetramethyl-1,3,2-dioxaborolan-2-yl)naphthalen-2-ol (500 mg, 1.5 equiv), 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (500 mg, 1 equiv) and Cs₂CO₃ (1 M in H₂O, 3.42 mL, 3.0 equiv) in methoxycyclopentane (10 mL) was added CataCXium A Pd G3 (83 mg, 0.1 equiv). The mixture was stirred at 80° C. for 5 hours under N₂ atmosphere. The mixture was diluted with H₂O (5 mL) and extracted with ethyl acetate (4×10 mL). The combined organic layer was dried over anhydrous Na₂SO₄, concentrated and purified with reversed phase flash chromatography [water (FA, 0.1%)/acetonitrile=3/1 to 2/1] to afford the tittle compound (0.38 g, 53% yield) as a light-yellow solid; LCMS (ESI, M+1): m/z=561.3.

Step I. (1R,5R,6R)-3-(8-fluoro-2-(((2R,7aS)-2-fluorohexahydro-1H-pyrrolizin-7a-yl)methoxy)-7-(3-hydroxy-5-methylnaphthalen-1-yl)pyrido[4,3-d]pyrimidin-4-yl)-3-azabicyclo[3.2.1]octan-6-ol: To a mixture of 4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-8-methylnaphthalen-2-ol (150 mg, 1.0 equiv) and (1R,5R,6R)-3-azabicyclo[3.2.1]octan-6-ol (55 mg, 1.6 equiv) in DMF (0.7 mL) was added DIPEA (69.2 mg, 2.0 equiv) and 4 Å molecular sieves (20 mg). The mixture was stirred at 40° C. for 40 hours under N₂ atmosphere. The mixture was filtered and purified with prep-HPLC [column: Phenomenex luna C18 150×25 mm×10 um; mobile phase: water (FA)-ACN; B %: 17%-37%, 7 minutes] to afford the tittle compound (66.0 mg, 40% yield, 0.33FA) as a yellow solid; ¹H NMR (400 MHZ, methanol-d4) δ=9.27 (s, 1H), 7.45 (d, J=2.0 Hz, 1H), 7.35 (br d, J=8.4 Hz, 1H), 7.29 (br d, J=6.8 Hz, 1H), 7.21 (d, J=2.0 Hz, 1H), 7.12 (dd, J=7.2, 8.0 Hz, 1H), 5.39 (d, J=53.2 Hz, 1H), 4.78 (br d, J=12.4 Hz, 1H), 4.45-4.42 (m, 1H), 4.36-4.32 (m, 2H), 3.77 (br d, J=12.4 Hz, 1H), 3.64-3.33 (m, 5H), 3.16 (dt, J=5.2, 9.6 Hz, 1H), 2.65 (s, 3H), 2.53-2.32 (m, 3H), 2.30-2.05 (m, 5H), 2.03-1.75 (m, 3H), 1.42-1.34 (m, 1H); LCMS (ESI, M+1): m/z=588.2.

### Example 748

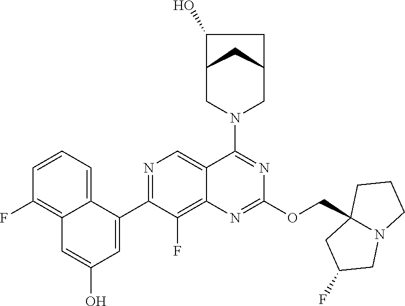 [SMILES: Oc1cc(-c2ncc3c(N4C[C@@H]5C[C@H](C4)[C@H](O)C5)nc(OC[C@@]45CCCN4C[C@H](F)C5)nc3c2F)c2cccc(F)c2c1]

### (1R,5R,6R)-3-(8-fluoro-7-(5-fluoro-3-hydroxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

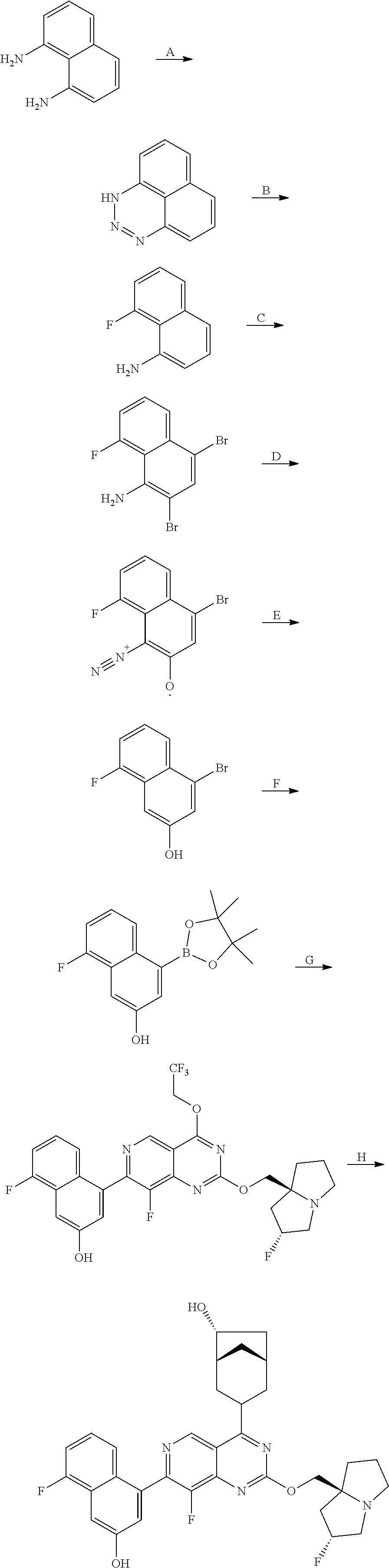

Step A. 1H-naphtho[1,8-de][1,2,3]triazine: To a solution of naphthalene-1,8-diamine (8.50 g, 1.0 equiv) in MeCN (100 mL) was added AcOH (40 mL) and a solution of NaNO₂ (7.41 g, 2.0 equiv) in H₂O (40 mL) at 0° C. Then the mixture was stirred at 0° C. for 1 hour. The reaction mixture was diluted with H₂O (50 mL) and extracted with EtOAc (3×50 mL). The combined organic layers were washed with brine (50 mL), dried over Na₂SO₄, filtered and concentrated to afford the title compound (9.50 g. 97% yield) as a brown solid; LCMS (ESI, M+1): m/z=170.1.

Step B. 8-fluoronaphthalen-1-amine: A solution of 1H-naphtho[1,8-de][1,2,3]triazine (8.50 g, 1.0 equiv) in pyridine hydrofluoride (62.3 g, 12.5 equiv) was stirred at 90° C. for 2 hours. The mixture was poured into aqueous sodium bicarbonate solution (500 mL) and extracted with EtOAc (3×300 mL). The combined organic layer was washed with brine (300 mL), dried over Na₂SO₄, then the mixture was filtered, concentrated and purified by column chromatagraphy [SiO₂, petroleum ether/ethyl acetate 1:0 to 30:1] to afford the title compound 8-fluoronaphthalen-1-amine (5.90 g, 73% yield) as a brown solid. ¹H NMR (400 MHZ, DMSO-d₆) δ=7.52 (d, J=8.4 Hz, 1H), 7.34-7.28 (m, 1H), 7.27-7.21 (m, 1H), 7.12-7.06 (m, 1H), 7.06-6.99 (m, 1H), 6.74 (br d, J=7.6 Hz, 1H), 5.74 (br s, 2H).

Step C. 2,4-dibromo-8-fluoronaphthalen-1-amine: To a solution of Br₂ (12.2 g, 2.2 equiv) in AcOH (44.1 g, 21 equiv) was added a solution of 8-fluoronaphthalen-1-amine (5.60 g, 1.0 equiv) in AcOH (14.7 g, 7.0 equiv) at 25° C. The reaction mixture was stirred at 25° C. for 2 hours. The reaction mixture was filtered. The filter cake was washed with AcOH (60 mL), then added into 20% aqueous of NaOH (50 mL). The mixture was stirred for 20 minutes and filtered. The solid was washed with water (50 mL) and dried to afford the title compound 2,4-dibromo-8-fluoro-naphthalen-1-amine (7.2 g, crude) as a brown solid. ¹H NMR (400 MHZ, DMSO-d₆) δ=7.85 (s, 1H), 7.79 (d, J=8.4 Hz, 1H), 7.57 (dt, J=5.6, 8.0 Hz, 1H), 7.32 (dd, J=7.6, 14.8 Hz, 1H), 5.92 (br d, J=2.8 Hz, 2H).

Step D. 4-bromo-1-diazonio-8-fluoro-naphthalen-2-olate: To a solution of 2,4-dibromo-8-fluoro-naphthalen-1-amine (7.10 g, 1.0 equiv) in AcOH (100 mL) and propionic acid (17.6 g, 10.7 equiv) was added NaNO₂ (1.84 g. 1.2 equiv) portionwise at 5-8° C. The reaction mixture was stirred at 5-8° C. for 1 hour. The reaction mixture was poured into ice-water (500 mL), the generated solid was filtered, washed with water (2×200 mL) and dried to afford the title compound 4-bromo-1-diazonio-8-fluoro-naphthalen-2-olate (6.0 g, 72% yield) as a brown solid. ¹H NMR (400 MHZ, DMSO-d₆) δ=7.77 (d, J=8.0 Hz, 1H), 7.51 (br dd, J=8.0, 11.2 Hz, 1H), 7.43-7.37 (m, 1H), 7.21 (s, 1H).

Step E. 4-bromo-8-fluoronaphthalen-2-ol: To a solution of 4-bromo-1-diazonio-8-fluoro-naphthalen-2-olate (5.50 g, 1.0 equiv) in EtOH (50 mL) was added NaBH₄(2.22 g, 2.8 equiv) at 0° C. The mixture was stirred at 20° C. for 2 hours. The mixture was quenched with saturated NH₄Cl aqueous solution (50 mL), then extracted with EtOAc (3×80 mL). The combined organic layers were washed with brine (50 mL), dried over Na₂SO₄, concentrated and purified by reversed phase flash chromatography [C18, 0.1% formic acid condition] to afford the title compound (1.35 g, 5.42 mmol, 26% yield, 96% purity) as a brown solid. ¹H NMR (400 MHZ, DMSO-d₆) δ=10.44 (br s, 1H), 7.79 (d, J=8.4 Hz, 1H), 7.54 (d, J=2.0 Hz, 1H), 7.41-7.30 (m, 2H), 7.28 (d, J=1.6 Hz, 1H).

Step F. 8-fluoro-4-(4,4,5,5-tetramethyl-1,3,2-dioxaborolan-2-yl)naphthalen-2-ol: To a solution of 4-bromo-8-fluoro-naphthalen-2-ol (0.50 g, 1.0 equiv), Bis(pinacolato)diboron (1.05 g, 2.0 equiv) in DMSO (20 mL) were added KOAc (610 mg, 3.0 equiv) and Pd(dppf)Cl₂ (152 mg, 0.1 equiv). The reaction mixture was stirred at 75° C. for 3 hours under N₂. The reaction mixture was diluted with water (20 mL) and extracted with EtOAc (3×30 mL). The combined organic layers were washed with brine (30 mL), dried over Na₂SO₄, concentrated and purified by column chromatography [SiO₂, Petroleum ether/Ethyl acetate 50:1 to 20:1] to afford the title compound (410 mg, 58% yield) as a white solid. ¹H NMR (400 MHZ, DMSO-d₆) δ=10.01 (s, 1H), 8.32 (d, J=8.4 Hz, 1H), 7.63 (br s, 1H), 7.35 (br s, 1H), 7.31-7.19 (m, 2H), 1.36 (s, 12H).

Step G. 8-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol: To a solution of 8-fluoro-4-(4,4,5,5-tetramethyl-1,3,2-dioxaborolan-2-yl)naphthalen-2-ol (300 mg, 1.2 equiv) and 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (381 mg, 1.0 equiv) in Cyclopentyl Methyl Ether (12 mL) were added Cs₂CO₃ (1.5 M, 3.0 equiv) and AdanBuP Pd G3 (63.2 mg, 0.1 equiv). The mixture was stirred at 100° C. for 3 hours under N₂. The reaction mixture was diluted with water (30 mL) and extracted with EtOAc (3×30 mL). The combined organic layers were washed with brine (40 mL), dried over Na₂SO₄, concentrated and purified by column chromatography [SiO₂, Petroleum ether/Ethyl acetate 3:1 to 1:2] to afford the title compound (188 mg, 30% yield) as a brown solid. ¹H NMR (400 MHZ, DMSO-d₆) δ=10.35 (s, 1H), 9.26 (s, 1H), 7.42 (d, J=2.0 Hz, 1H), 7.35-7.31 (m, 2H), 7.31-7.20 (m, 2H), 5.41-5.35 (m, 2H), 4.30-4.17 (m, 2H), 3.31 (s, 2H), 3.14-3.06 (m, 2H), 2.89-2.80 (m, 1H), 2.10-2.01 (m, 2H), 1.92-1.75 (m, 4H).

Step G. (1R,5R,6R)-3-(8-fluoro-7-(5-fluoro-3-hydroxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 8-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (90.0 mg, 1.0 equiv) and (1S,5S,6R)-3-azabicyclo[3.2.1]octan-6-ol (30.4 mg, 1.2 equiv, HCl) in DMF (1.0 mL) was added DIPEA (103 mg, 5.0 equiv) and 4 Å molecular sieves (90 mg). The mixture was stirred at 60° C. for 5 hours. The reaction mixture was diluted with water (10 mL) and extracted with EtOAc (3×10 mL). The combined organic layers were washed with brine (15 mL), dried over Na₂SO₄, concentrated and purified by prep-HPLC [Waters Xbridge 150×25 mm×5 μm; A: water (NH₄HCO₃), B: ACN; B %: 38%-68% over 9 min] and further re-purified by prep-HPLC [Phenomenex Synergi C18 150×25 mm×10 μm; A: water (FA), B: ACN; B %: 11%-41% over 10 min] to afford the title compound (40.0 mg, 42% yield) as a white solid (0.50 formic acid salt); ¹H NMR (400 MHZ, METHANOL-d₄) δ=9.29 (d, J=0.8 Hz, 1H), 8.51 (s, 1H), 7.47 (d, J=2.4 Hz, 1H), 7.34 (br d, J=8.0 Hz, 1H), 7.29 (d, J=2.8 Hz, 1H), 7.23-7.12 (m, 2H), 5.57-5.30 (m, 1H), 4.98-4.92 (m, 1H), 4.85-4.74 (m, 1H), 4.54-4.38 (m, 2H), 4.33 (td, J=4.8, 10.0 Hz, 1H), 3.78 (br dd, J=4.4, 12.4 Hz, 1H), 3.70-3.46 (m, 4H), 3.27-3.15 (m, 1H), 2.57-2.33 (m, 3H), 2.30-2.11 (m, 5H), 2.07-1.97 (m, 1H), 1.91 (s, 1H), 1.86-1.77 (m, 1H), 1.38 (br d, J=13.6 Hz, 1H). LCMS (ESI, M+1): m/z=592.4.

### Example 749

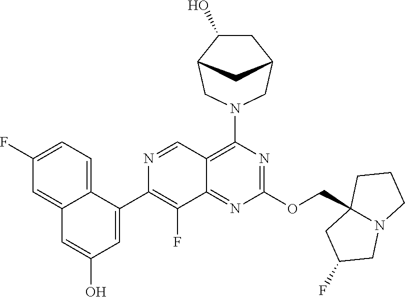 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccc(F)cc2c1]

### (1R,5R,6R)-3-(8-fluoro-7-(6-fluoro-3-hydroxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

The title compound was synthesized from 2-(3-fluorophenyl) acetic acid according to the 8-step procedure described for example 746 to produce the desired compound as a yellow solid (0.41 formic acid salt); ¹H NMR (400 MHZ, DMSO-d₆) δ=10.21 (br s, 1H), 9.39 (s, 1H), 8.21 (s, 1H), 7.69-7.52 (m, 2H), 7.28 (d, J=2.4 Hz, 1H), 7.24-7.08 (m, 2H), 5.43-5.17 (m, 1H), 4.75 (br d, J=13.2 Hz, 2H), 4.66-4.51 (m, 1H), 4.21-4.08 (m, 2H), 4.01 (dd, J=3.2, 10.4 Hz, 1H), 3.77-3.67 (m, 1H), 3.37-3.36 (m, 1H), 3.15-3.06 (m, 2H), 3.01 (s, 1H), 2.90-2.77 (m, 1H), 2.21-1.95 (m, 6H), 1.87-1.74 (m, 4H), 1.70-1.62 (m, 1H), 1.30-1.19 (m, 1H); LCMS (ESI, M+1): m/z=592.3.

### Example 750

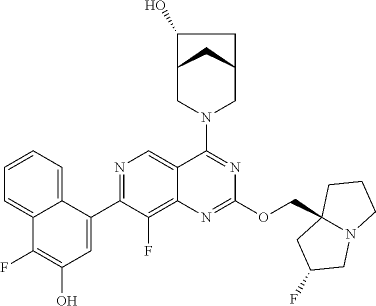 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1F]

### (1R,5R,6R)-3-(8-fluoro-7-(4-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

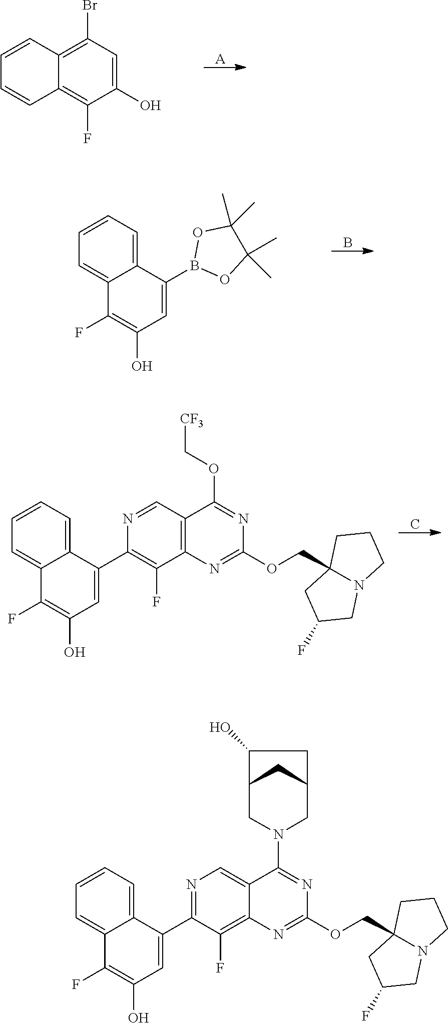

Step A. 1-fluoro-4-(4,4,5,5-tetramethyl-1,3,2-dioxaborolan-2-yl)naphthalen-2-ol: To a mixture of 4-bromo-1-fluoronaphthalen-2-ol (500 mg, 1.0 equiv), Bis(pinacolato)diboron (1.05 g, 2.0 equiv) and KOAc (407 mg, 2.0 equiv) in dioxane (10 mL) was added Pd(dppf)Cl₂ (152 mg, 0.1 equiv). The reaction was degassed and stirred at 90° C. for 2 hours under N₂ atmosphere. The reaction mixture was diluted with H₂O (50 mL) and EtOAc (50 mL), and the aqueous layer was extracted with EtOAc (30 mL×3). The combined organic layers were washed with brine (30 mL×2), dried over Na₂SO₄, filtered, concentrated and purified with column chromatography (SiO₂, Petroleum ether/Ethyl acetate 20:1 to 5:1) to afford the title compound (420 mg, 63% yield) as a white solid; ¹H NMR (400 MHZ, DMSO-d₆) δ=10.05 (s, 1H), 8.57 (br d, J=8.4 Hz, 1H), 7.91 (d, J=8.4 Hz, 1H), 7.77 (d, J=9.6 Hz, 1H), 7.53 (t, J=7.2 Hz, 1H), 7.49-7.38 (m, 1H), 1.36 (s, 12H).

Step B. 1-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol: To a mixture of 1-fluoro-4-(4,4,5,5-tetramethyl-1,3,2-dioxaborolan-2-yl)naphthalen-2-ol (49.2 mg, 1.5 equiv), 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (50.0 mg, 1.0 equiv) and Cs₂CO₃ (1.5 M, 228 μL,3.0 equiv) in CPME (1 mL) was added AdanBuP Pd G3 (16.6 mg, 0.2 equiv). The reaction was degassed and stirred at 100° C. for 2 hours under N₂ atmosphere (15 psi). The reaction mixture was diluted with H₂O (10 mL) and EtOAc (10 mL), then filtered and extracted with EtOAc (10 mL×3). The combined organic layers were washed with brine (10 mL×2), dried over Na₂SO₄, filtered, concentrated and purified by prep-TLC (SiO₂, MeCN) to afford the title compound (55.0 mg, 85% yield) as a yellow solid; ¹H NMR (400 MHZ, DMSO-d₆) δ=10.40 (s, 1H), 9.27 (s, 1H), 8.02 (d, J=8.8 Hz, 1H), 7.63-7.55 (m, 2H), 7.48-7.42 (m, 1H), 7.40-7.33 (m, 1H), 5.40 (br d, J=8.8 Hz, 2H), 4.32-4.16 (m, 2H), 3.13-3.04 (m, 2H), 2.96-2.79 (m, 2H), 2.19-2.09 (m, 2H), 1.93-1.81 (m, 3H), 1.73-1.62 (m, 1H), 1.23 (br s, 1H).

Step C. (1R,5R,6R)-3-(8-fluoro-7-(4-fluoro-3-hydroxynaphthalen-1-yl)-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a mixture of 1-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (50.0 mg, 1.0 equiv) and (1R,5R,6R)-3-azabicyclo[3.2.1]octan-6-ol (29.0 mg, 2.0 equiv, HCl) in DMF (0.25 mL) were added DIPEA (57.2 mg, 5.0 equiv) and 4 Å MS (20.0 mg). The mixture was stirred at 60° C. for 6 hours. The reaction mixture was filtered and purified with prep-HPLC (column: Phenomenex luna C18 150*25 mm*10 um; mobile phase: [water (FA)-ACN]; B %: 15%-45%, 10 min) to afford the title compound (4.83 mg, 9% yield) as a yellow solid (0.18 formic acid salt); ¹H NMR (400 MHZ, DMSO-d₆) δ=11.06-9.84 (m, 1H), 9.39 (s, 1H), 8.20 (s, 1H), 8.00 (d, J=8.4 Hz, 1H), 7.72-7.51 (m, 2H), 7.50-7.29 (m, 2H), 5.43-5.13 (m, 1H), 4.95-4.68 (m, 2H), 4.60 (br d, J=12.0 Hz, 1H), 4.24-4.09 (m, 2H), 4.02 (dd, J=4.0, 10.4 Hz, 1H), 3.72 (br d, J=12.0 Hz, 1H), 3.13-3.00 (m, 3H), 2.86-2.79 (m, 1H), 2.33 (br d, J=0.8 Hz, 1H), 2.21-1.98 (m, 5H), 1.92-1.74 (m, 4H), 1.72-1.61 (m, 1H), 1.25 (br d, J=12.8 Hz, 1H); LCMS (ESI, M+1): m/z=592.4.

### Example 751

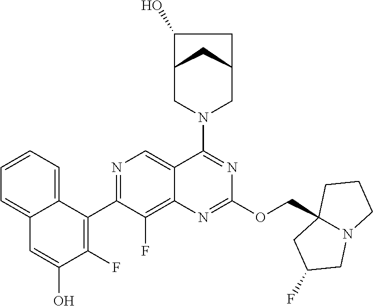 [SMILES: Oc1cc2ccccc2c(-c2ncc3c(N4C[C@@H]5C[C@H](C4)[C@H](O)C5)nc(OC[C@@]45CCCN4C[C@H](F)C5)nc3c2F)c1F]

### (1R,5R,6R)-3-(8-fluoro-7-(2-fluoro-3-hydroxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

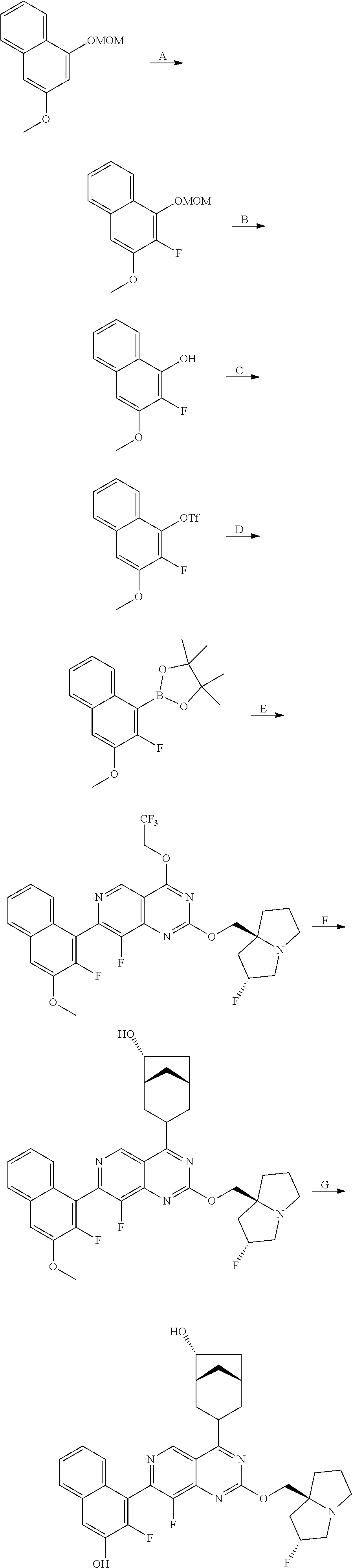

Step A. 2-fluoro-3-methoxy-1-(methoxymethoxy)naphthalene; To a solution of 3-methoxy-1-(methoxymethoxy)naphthalene (7.50 g, 1.0 equiv) in THF (75 mL) was slowly added n-BuLi (2.5 M, 2.0 equiv) and tetramethylethylenediamine (3.99 g, 1.0 equiv) under N₂ and the mixture was stirred at 0° C. for 2 hours. Then a solution of NFSI (21.7 g, 2.0 equiv) in THF (10 mL) was added to the above reaction solution and the mixture was stirred at 20° C. for 0.5 hour. The mixture was slowly quenched with water (200 mL) at 0° C., and extracted with ethyl acetate (3×100 mL). The combined organic layers were dried over anhydrous sodium sulfate and concentrated under vacuum to give a residue. The residue was purified by column chromatography (SiO₂, petroleum ether/ethyl acetate 30:1 to 10:1) to afford the title compound (3.1 g, 39% yield) as a yellow oil; ¹H NMR (400 MHZ, CHLOROFORM-d) δ=8.13-8.07 (m, 1H), 7.74-7.69 (m, 1H), 7.48-7.38 (m, 2H), 7.01 (d, J=8.0 Hz, 1H), 5.36 (d, J=0.8 Hz, 2H), 4.01 (s, 3H), 3.66 (s, 3H).

Step B. 2-fluoro-3-methoxynaphthalen-1-ol: To a solution of 2-fluoro-3-methoxy-1-(methoxymethoxy)naphthalene (3.10 g, 1.0 equiv) in DCM (20 mL) was added TFA (30.8 g, 21 equiv) at 0° C. The reaction mixture was stirred at 0° C. for 0.5 hour. The reaction mixture was diluted with water (30 mL), extracted with EtOAc (10 mL), the combined organic phase was washed with brine (20 mL), dried over anhydrous sodium sulfate, filtered, concentrated and purified by column chromatography (SiO₂, petroleum ether/ethyl acetate 30:1 to 10:1) to afford the title compound (1.50 g, 59% yield) as a red oil; ¹H NMR (400 MHZ, CHLOROFORM-d) δ=8.12-8.08 (m, 1H), 7.68 (d, J=7.6 Hz, 1H), 7.49-7.34 (m, 2H), 6.83 (d, J=7.6 Hz, 1H), 5.63 (d, J=4.8 Hz, 1H), 4.00 (s, 3H).

Step C. 2-fluoro-3-methoxynaphthalen-1-yl trifluoromethanesulfonate: To a solution of 2-fluoro-3-methoxy-naphthalen-1-ol (1.00 g, 1.0 equiv) in DCM (10 mL) at 0° C. was added TEA (1.05 g, 2.0 equiv) and Tf₂O (2.20 g, 1.5 equiv). The mixture was stirred at 0° C. for 0.5 hour. The reaction mixture was diluted with water (40 mL), extracted with EtOAc (20 mL). The combined organic phase was washed with brine (20 mL), dried over anhydrous sodium sulfate, concentrated and purified by column chromatography (SiO₂, petroleum ether/ethyl acetate 30:1 to 10:1) to afford the title compound (1.56 mg, 92% yield) as a yellow oil; ¹H NMR (400 MHZ, CHLOROFORM-d) δ=7.99-7.92 (m, 1H), 7.81-7.74 (m, 1H), 7.59-7.51 (m, 2H), 7.27-7.24 (m, 1H), 4.04 (s, 3H).

Step D. 2-(2-fluoro-3-methoxynaphthalen-1-yl)-4,4,5,5-tetramethyl-1,3,2-dioxaborolane: A mixture of (2-fluoro-3-methoxy-1-naphthyl)trifluoromethanesulfonate (500 mg, 1.0 equiv), bis(pinacolato)diboron (1.57 g, 4.0 equiv), KOAc (454 mg, 3.0 equiv), and Pd(dppf)Cl₂ (113 mg, 0.1 equiv) in dioxane (5 mL) was degassed and stirred at 100° C. for 16 hours under N₂ atmosphere. The reaction mixture was diluted with water (40 mL), extracted with EtOAc (2×30 mL). The combined organic phase was washed with brine (20 mL), dried over anhydrous sodium sulfate, concentrated and purified by reversed phase flash chromatography [C18, 0.1% formic acid condition] to afford the title compound (270 mg, 44% yield) as a black oil; ¹H NMR (400 MHZ, CHLOROFORM-d) δ=8.23-8.18 (m, 1H), 7.74-7.68 (m, 1H), 7.43-7.37 (m, 2H), 3.98 (s, 3H), 1.47 (s, 12H); LCMS (ESI, M+1): m/z=303.1.

Step E. 8-fluoro-7-(2-fluoro-3-methoxynaphthalen-1-yl)-2-(((2R,7aS)-2-fluorohexahydro-1H-pyrrolizin-7a-yl)methoxy)-4-(2,2,2-trifluoroethoxy)pyrido[4,3-d]pyrimidine: A mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (200 mg, 1.0 equiv), 2-(2-fluoro-3-methoxy-1-naphthyl)-4,4,5,5-tetramethyl-1,3,2-dioxaborolane (207 mg, 1.5 equiv), CataCXium A Pd G3 (33.2 mg, 0.1 equiv) and Cs₂CO₃ (1.5 M, 3.0 equiv) in methoxycyclopentane (4 mL) was degassed and stirred at 85° C. for 3 hour under N₂ atmosphere. The reaction mixture was diluted with water (40 mL), extracted with EtOAc (2×30 mL). The combined organic phase was washed with brine (50 mL), dried over anhydrous sodium sulfate, concentrated to give a residue and purified by pre-TLC (Dichloromethane/Methanol 10:1) to afford the title compound (140 mg, 50% yield) as a white solid; LCMS (ESI, M+1): m/z=579.3.

Step F. (1R,5R,6R)-3-(8-fluoro-7-(2-fluoro-3-methoxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 8-fluoro-7-(2-fluoro-3-methoxynaphthalen-1-yl)-2-(((2R,7aS)-2-fluorohexahydro-1H-pyrrolizin-7a-yl)methoxy)-4-(2,2,2-trifluoroethoxy)pyrido[4,3-d]pyrimidine (160 mg, 1.0 equiv) in DMF (1.0 mL) was added (1R,5R,6R)-3-azabicyclo[3.2.1]octan-6-ol (70.4 mg, 2.0 equiv), DIPEA (107 mg, 3.0 equiv) and 4 Å molecular sieves (20 mg). The reaction mixture was stirred at 40° C. for 4 hours. The mixture was filtered and purified by reversed phase flash chromatography [C18, 0.1% formic acid] to afford the title compound (160 mg, 96% yield) as a white solid; LCMS (ESI, M+1): m/z=606.3.

Step G. (1R,5R,6R)-3-(8-fluoro-7-(2-fluoro-3-hydroxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of (1R,5R,6R)-3-(8-fluoro-7-(2-fluoro-3-methoxynaphthalen-1-yl)-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (100 mg, 1.0 equiv) in DCM (1 mL) was added dropwise BBr₃ (207 mg, 5.0 equiv) in DCM (0.5 mL) at 0° C. The resulting mixture was stirred at 0° C. for 5 hours. The mixture was poured into ice-cooled saturated NaHCO₃ solution (10 mL) extracted with EtOAc (2×5 mL), the combined organic phase was washed with brine (10 mL), dried over anhydrous sodium sulfate, concentrated and purified by prep-HPLC [column: Waters Xbridge 150×25 mm×5 μm; A: water (NH₄HCO₃), B: ACN, B %: 32%-62% over 8 min] to afford the title compound (20.9 mg, 21% yield) as a white solid; Column: Chiralpak IC-3 50×4.6 mm I.D., 3 um Mobile phase: Phase A for CO₂, and Phase B for MeOH+CAN (0.05% DEA); Gradient elution: 40% MeOH+ACN (0.05% DEA) in CO₂; Flow rate: 3 mL/min; Detector: PDA; Column Temp: 35C; Back Pressure: 100Bar. ¹H NMR (400 MHZ, METHANOL-d4) δ=9.32 (d, J=2.4 Hz, 1H), 7.79 (d, J=8.0 Hz, 1H), 7.47-7.34 (m, 3H), 7.30-7.23 (m, 1H), 5.43-5.21 (m, 1H), 5.01-4.90 (m, 1H), 4.79-4.74 (m, 1H), 4.39-4.27 (m, 2H), 4.26-4.20 (m, 1H), 3.79-3.76 (m, 1H), 3.48 (br d, J=12.6 Hz, 1H), 3.29-3.12 (m, 3H), 3.07-2.95 (m, 1H), 2.45-2.37 (m, 1H), 2.35-2.10 (m, 5H), 2.05-1.86 (m, 4H), 1.85-1.76 (m, 1H), 1.46-1.36 (m, 1H); LCMS (ESI, M+1): m/z=592.4;

### Example 752

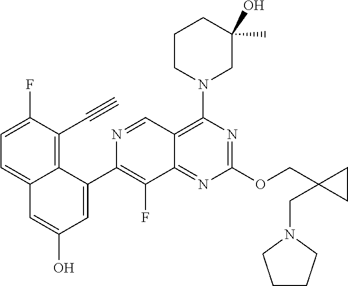 [SMILES: C#Cc1c(F)ccc2cc(O)cc(-c3ncc4c(N5CCC[C@@](C)(O)C5)nc(OCC5(CN6CCCC6)CC5)nc4c3F)c12]

### (R)-1-(7-(8-ethynyl-7-fluoro-3-hydroxynaphthalen-1-yl)-8-fluoro-2-((1-(pyrrolidin-1-ylmethyl)cyclopropyl)methoxy)pyrido[4,3-d]pyrimidin-4-yl)-3-methylpiperidin-3-ol

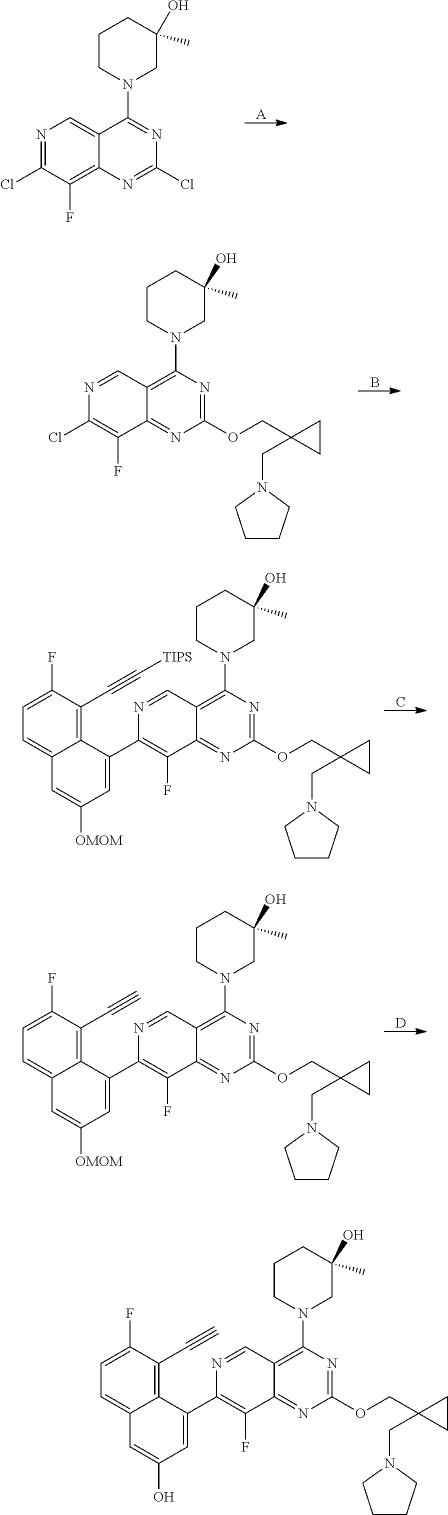

Step A (R)-1-(7-chloro-8-fluoro-2-((1-(pyrrolidin-1-ylmethyl)cyclopropyl)methoxy)pyrido[4,3-d]pyrimidin-4-yl)-3-methylpiperidin-3-ol: To a solution of (R)-1-(2,7-dichloro-8-fluoropyrido[4,3-d]pyrimidin-4-yl)-3-methylpiperidin-3-ol (1.10 g, 1.0 equiv) in MeCN (10 mL) were added (1-(pyrrolidin-1-ylmethyl)cyclopropyl)methanol (567 mg, 1.1 equiv) 4 Å molecular sieves (100 mg) and Cs₂CO₃ (3.25 g, 3.0 equiv). The mixture was stirred at 60° C. for 1 hour. The reaction mixture was partitioned between ethyl acetate (20.0 mL) and water (10.0 mL). The organic phase was separated, concentrated under reduced pressure to afford the title compound (850 mg, 28% yield); LCMS (ESI, M+1): m/z=450.1

Step B (R)-1-(8-fluoro-7-(7-fluoro-3-(methoxymethoxy)-8-((triisopropylsilyl)ethynyl)naphthalen-1-yl)-2-((1-(pyrrolidin-1-ylmethyl)cyclopropyl)methoxy)pyrido[4,3-d]pyrimidin-4-yl)-3-methylpiperidin-3-ol: To a solution of (R)-1-(7-chloro-8-fluoro-2-((1-(pyrrolidin-1-ylmethyl)cyclopropyl)methoxy)pyrido[4,3-d]pyrimidin-4-yl)-3-methylpiperidin-3-ol (850 mg, 1.00 equiv) in dioxane (10.0 mL) and H₂O (2.00 mL) were added K₃PO₄ (802 mg, 2.0 equiv), ((2-fluoro-6-(methoxymethoxy)-8-(4,4,5,5-tetramethyl-1,3,2-dioxaborolan-2-yl)naphthalen-1-yl)ethynyl)triisopropylsilane (968 mg, 1.0 equiv) and CataCXium A Pd G3 (138 mg, 0.1 equiv). The mixture was stirred at 90° C. for 2 hours. The reaction mixture was partitioned between ethyl acetate (50 mL) and water (30 mL). The organic phase was separated, and concentrated under reduced pressure to give a residue. The residue was purified by prep-TLC (SiO₂, dichloromethane/methyl alcohol 10:1) to afford the title compound (310 mg. 20% yield) as a yellow solid; LCMS (ESI, M+1): m/z=800.4

Step C (R)-1-(7-(8-ethynyl-7-fluoro-3-(methoxymethoxy)naphthalen-1-yl)-8-fluoro-2-((1-(pyrrolidin-1-ylmethyl)cyclopropyl)methoxy)pyrido[4,3-d]pyrimidin-4-yl)-3-methylpiperidin-3-ol: To a solution of (R)-1-(8-fluoro-7-(7-fluoro-3-(methoxymethoxy)-8-((triisopropylsilyl)ethynyl)naphthalen-1-yl)-2-((1-(pyrrolidin-1-ylmethyl)cyclopropyl)methoxy)pyrido[4,3-d]pyrimidin-4-yl)-3-methylpiperidin-3-ol (300 mg, 1.0 equiv) in DMF (8.0 mL) was added CsF (570 mg, 10 equiv). The mixture was stirred at 20° C. for 2 hours. The reaction mixture was concentrated under reduced pressure to give a residue. The residue was purified by prep-TLC (SiO₂, dichloromethane/methyl alcohol 10:1) to afford the title compound (110 mg, 46% yield) as a yellow solid; LCMS (ESI, M+1): m/z=644.4

Step D (R)-1-(7-(8-ethynyl-7-fluoro-3-hydroxynaphthalen-1-yl)-8-fluoro-2-((1-(pyrrolidin-1-ylmethyl)cyclopropyl)methoxy)pyrido[4,3-d]pyrimidin-4-yl)-3-methylpiperidin-3-ol: To a solution of (R)-1-(7-(8-ethynyl-7-fluoro-3-(methoxymethoxy)naphthalen-1-yl)-8-fluoro-2-((1-(pyrrolidin-1-ylmethyl)cyclopropyl)methoxy)pyrido[4,3-d]pyrimidin-4-yl)-3-methylpiperidin-3-ol (100 mg, 1.0 equiv) in EtOAc (3.0 mL) was added HCl/EA (4 M, 3.0 mL). The mixture was stirred at 25° C. for 1 hour. The reaction mixture was concentrated and purified by prep-HPLC (column: Phenomenex luna C18 150×25 mm×10 um; mobile phase: [water (FA)-ACN]; B %: 15%-45%, 10 min) and lyophilized to afford the title compound (13.1 mg, 13% yield) as a yellow solid (0.60 formic acid salt); ¹H NMR (400 MHZ, CD₃OD) δ=9.30-9.04 (m, 1H), 8.56 (s, 1H), 7.94-7.85 (m, 1H), 7.42-7.21 (m, 3H), 4.73-4.53 (m, 3H), 4.41-4.26 (m, 2H), 3.72-3.37 (m, 3H), 3.22-2.98 (m, 5H), 2.32-2.11 (m, 1H), 1.99 (br s, 4H), 1.92-1.64 (m, 3H), 1.33-1.21 (m, 3H), 0.88 (br s, 2H), 0.82 (s, 2H); LCMS (ESI, M−1): m/z=600.4

### Example 753

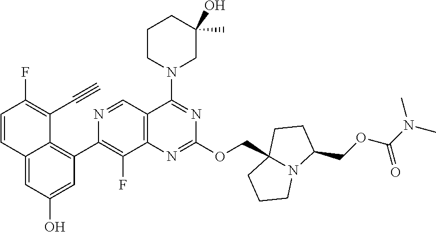 [SMILES: C#Cc1c(F)ccc2cc(O)cc(-c3ncc4c(N5CCC[C@@](C)(O)C5)nc(OC[C@]56CCCN5[C@H](COC(=O)N(C)C)CC6)nc4c3F)c12]

### ((3S,7aR)-7a-(((7-(8-ethynyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate

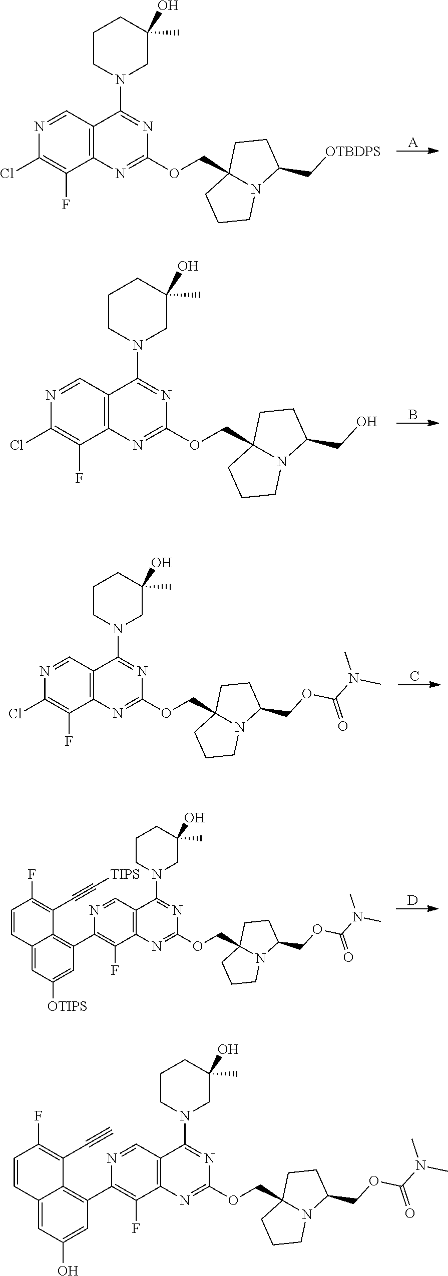

Step A. (R)-1-(7-chloro-8-fluoro-2-(((3S,7aR)-3-(hydroxymethyl)hexahydro-1H-pyrrolizin-7a-yl)methoxy)pyrido[4,3-d]pyrimidin-4-yl)-3-methylpiperidin-3-ol: To a mixture of (R)-1-(2-(((3S,7aR)-3-(((tert-butyldiphenylsilyl)oxy)methyl)hexahydro-1H-pyrrolizin-7a-yl)methoxy)-7-chloro-8-fluoropyrido[4,3-d]pyrimidin-4-yl)-3-methylpiperidin-3-ol (5.00 g, 1.0 equiv) in DMF (50 mL) was added CsF (16.2 g, 15 equiv) at 0° C. under N₂. The mixture was stirred at 25° C. for 1 hour. The residue was poured into ice-water (100 mL) and stirred for 5 minutes. The aqueous phase was extracted with ethyl acetate (100 mL×3). The combined organic phases were washed with brine (100 mL), dried with anhydrous Na₂SO₄, filtered and concentrated under vacuum. The residue was purified by silica gel chromatography [DCM/MeOH 10:1 to 5:1] to afford the title compound (3.00 g, 91% yield) as a colorless oil.

Step B. ((3S,7aR)-7a-(((7-chloro-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: To a mixture of (R)-1-(7-chloro-8-fluoro-2-(((3S,7aR)-3-(hydroxymethyl)hexahydro-1H-pyrrolizin-7a-yl)methoxy)pyrido[4,3-d]pyrimidin-4-yl)-3-methylpiperidin-3-ol (1.00 g, 1.0 equiv), TEA (568 mg, 3.0 equiv) in DCM (10 mL) at 0° C. under N₂ was added (4-nitrophenyl) carbonochloridate (565 mg, 1.5 equiv). The mixture was stirred at 25° C. for 2 hours. Then N-methylmethanamine (213 mg, 1.5 equiv, HCl) was added dropwise at 0° C. under N₂. The reaction mixture was stirred at 25° C. for 2 hours. The residue was poured into ice-water (15 mL) and stirred for 1 min. The aqueous phase was extracted with DCM (25 mL×3). The combined organic phases were washed with brine (35 mL), dried with anhydrous Na₂SO₄, filtered and concentrated under vacuum. The residue was purified by column chromatography (SiO₂, Petroleum ether/Ethyl acetate 3:1 to 1:1) to afford the title compound (3.00 g, 91% yield) as a yellow oil; ¹H NMR (400 MHZ, DMSO-d6) δ=9.04-8.96 (m, 1H), 5.74 (s, 1H), 4.75-4.63 (m, 1H), 4.34 (br d, J=13.2 Hz, 1H), 4.03-3.95 (m, 3H), 3.90 (dd, J=5.2, 10.6 Hz, 1H), 3.79 (dd, J=6.4, 10.5 Hz, 1H), 2.97-2.87 (m, 2H), 2.82 (br d, J=13.6 Hz, 6H), 2.70-2.62 (m, 1H), 1.97-1.88 (m, 2H), 1.85-1.73 (m, 4H), 1.71-1.49 (m, 7H), 1.14 (s, 3H).

Step C.: ((3S,7aR)-7a-(((8-fluoro-7-(7-fluoro-8-((triisopropylsilyl)ethynyl)-3-((triisopropylsilyl)oxy)naphthalen-1-yl)-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: ((3S,7aR)-7a-(((7-chloro-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate (400 mg, 1.0 equiv), [6-fluoro-4-(4,4,5,5-tetramethyl-1,3,2-dioxaborolan-2-yl)-5-(2-triisopropylsilylethynyl)-2-naphthyl]oxy-triisopropyl-silane (698 mg, 1.5 equiv), K₃PO₄(474 mg, 3.0 equiv), and Pd(dppf)Cl₂ (48.5 mg, 74.5 μmol, 0.1 equiv) in dioxane (10 mL) and H₂O (2.5 mL) was degassed and then heated to 80° C. for 2 hours under N₂. The residue was poured into ice-water (5 mL) and stirred for 5 minutes. The aqueous phase was extracted with ethyl acetate (10 mL×3). The combined organic phases were washed with brine (10 mL), dried with anhydrous Na₂SO₄, filtered and concentrated under vacuum. The mixture was filtered and purified by prep-HPLC [C18, 0.1% formic acid condition] to afford the title compound (180 mg, 24% yield) as a yellow solid.

Step D. ((3S,7aR)-7a-(((7-(8-ethynyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate: To a mixture of ((3S,7aR)-7a-(((8-fluoro-7-(7-fluoro-8-((triisopropylsilyl)ethynyl)-3-((triisopropylsilyl)oxy)naphthalen-1-yl)-4-((R)-3-hydroxy-3-methylpiperidin-1-yl)pyrido[4,3-d]pyrimidin-2-yl)oxy)methyl)hexahydro-1H-pyrrolizin-3-yl)methyl dimethylcarbamate (80.0 mg, 1.0 equiv) in DMF (1 mL) was added CsF (182 mg, 15 equiv) at 25° C. under N₂. The mixture was stirred at 40° C. for 12 hours. The residue was poured into ice-water (10 mL) and stirred for 2 minutes. The aqueous phase was extracted with ethyl acetate (15 mL×3). The combined organic phases were filtered and concentrated and purified by prep-HPLC [C18, 0.1% formic acid condition] and lyophilized to afford the title compound (50.6 mg, 85% yield) as a yellow solid (0.25 formic acid salt); ¹H NMR (400 MHZ, METHANOL-d4) δ=9.24-9.08 (m, 1H), 8.54 (s, 1H), 7.92-7.85 (m, 1H), 7.39-7.31 (m, 2H), 7.30-7.23 (m, 1H), 4.72-4.55 (m, 1H), 4.46-4.28 (m, 3H), 4.25-4.17 (m, 1H), 4.11-4.02 (m, 1H), 3.70-3.60 (m, 2H), 3.51-3.38 (m, 1H), 3.28-3.19 (m, 1H), 3.12-3.01 (m, 1H), 2.97-2.84 (m, 6H), 2.36-1.71 (m, 12H), 1.35-1.24 (m, 3H); LCMS (ESI, M+1): m/z=687.5.

### Example 754

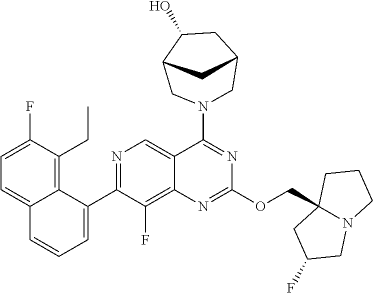 [SMILES: CCc1c(F)ccc2cccc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

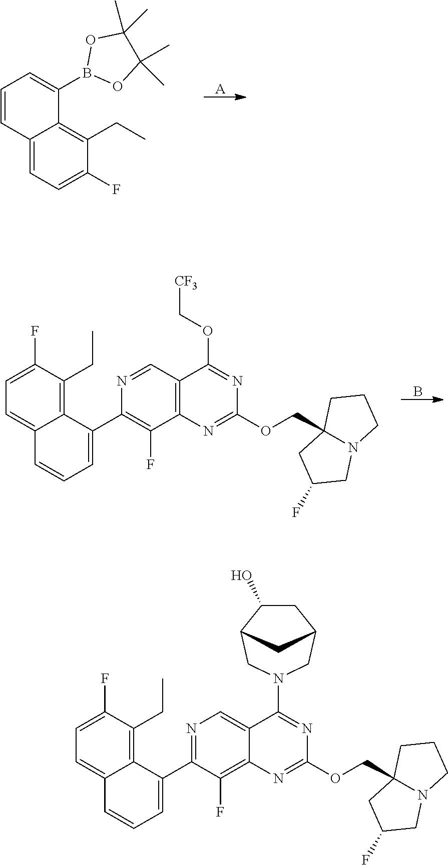

Step A. 7-(8-ethyl-7-fluoronaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: A mixture of 2-(8-ethyl-7-fluoronaphthalen-1-yl)-4,4,5,5-tetramethyl-1,3,2-dioxaborolane (150 mg, 1.3 equiv), 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (175 mg, 1.0 equiv), Ad₂n-BuP Pd G₃ (43.7 mg, 0.15 equiv), Cs₂CO₃ (391 mg, 3.0 equiv) in methoxycyclopentane (5.0 mL) was degassed and stirred at 100° C. for 3 hours under N₂ atmosphere. The reaction mixture was diluted with water (50 mL) and extracted with ethyl acetate (2×50 mL). The combined organic layers were washed with brine (50 mL), dried over anhydrous sodium sulfate, concentrated and purified with prep-TLC [SiO₂, dichloromethane/methanol 10:1] to afford the title compound (50.0 mg, 22% yield) as a yellow oil. LCMS [M+1]=577.2.

Step B. (1R,5R,6R)-3-(7-(8-ethyl-7-fluoronaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 7-(8-ethyl-7-fluoronaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (50.0 mg, 1.0 equiv) in DMF (0.5 mL) and acetonitrile (0.5 mL) was added (1R,5R,6R)-3-azabicyclo[3.2.1]octan-6-ol (13.2 mg, 1.2 equiv), DIPEA (33.6 mg, 3 equiv) and 4 Å molecular sieves (10.0 mg). The mixture was stirred at 25° C. for 5 hours. The reaction mixture was diluted with water (20 mL) and extracted with ethyl acetate (2×30 mL). The combined organic layers were washed with brine (20 mL), dried over anhydrous sodium sulfate, concentrated and purified with prep-HPLC [column: Phenomenex luna C18 150×25 mm×10 um; mobile phase: [water (FA)-ACN]; B %: 18%-48%, 10 minutes] to afford the title compound (4.30 mg, 8.13% yield) as a white solid (0.18 formic acid salt). ¹H NMR (400 MHZ, DMSO-d₆) δ=9.43-9.20 (m, 1H), 8.12 (d, J=8.0 Hz, 1H), 8.03 (dd, J=6.4, 8.8 Hz, 1H), 7.59 (s, 1H), 7.54-7.44 (m, 2H), 5.43-5.15 (m, 1H), 4.91-4.69 (m, 2H), 4.64-4.51 (m, 1H), 4.24-4.07 (m, 2H), 4.01 (d, J=10.0 Hz, 1H), 3.75 (br s, 1H), 3.09 (br d, J=7.6 Hz, 2H), 3.02 (s, 1H), 2.83 (br d, J=6.0 Hz, 1H), 2.69-2.65 (m, 1H), 2.34 (br d, J=1.6 Hz, 3H), 2.26-1.95 (m, 6H), 1.93-1.59 (m, 5H), 1.34-1.17 (m, 2H), 0.75 (dt, J=4.0, 7.2 Hz, 3H); LCMS [M+1]=604.4.

### Example 755

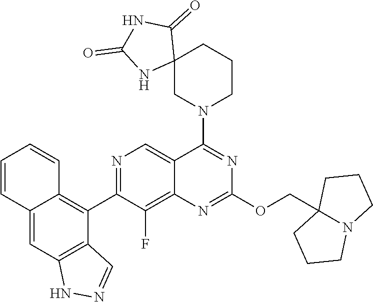 [SMILES: O=C1NC(=O)C2(CCCN(c3nc(OCC45CCCN4CCC5)nc4c(F)c(-c5c6ccccc6cc6[nH]ncc56)ncc34)C2)N1]

### 7-(7-(1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione

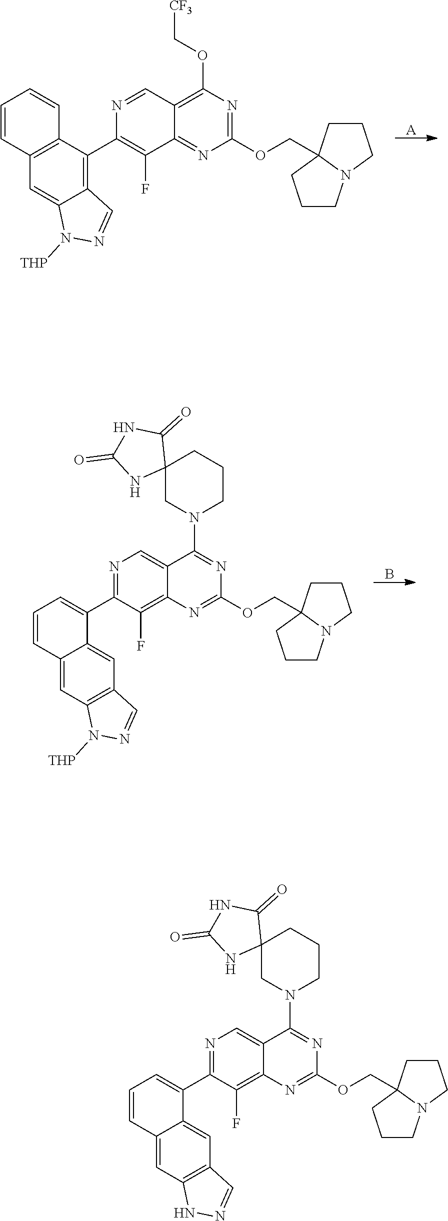

Step A. 7-(8-fluoro-2-((hexahydro-1H-pyrrolizin-7a-yl)methoxy)-7-(1-(tetrahydro-2H-pyran-2-yl)-1H-benzo[f]indazol-4-yl)pyrido[4,3-d]pyrimidin-4-yl)-1,3,7-triazaspiro[4.5]decane-2,4-dione: To a mixture of 4-(8-fluoro-2-((hexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole (50.0 mg, 1.0 equiv), DIPEA (101 mg, 10 equiv) and 4 Å molecular sieves (5 mg) in DMF (0.5 mL) was added 1,3,9-triazaspiro[4.5]decane-2,4-dione (19.9 mg, 1.5 equiv). The reaction was stirred at 40° C. for 12 hrs. The mixture was filtered, concentrated and purified by reversed phase flash chromatography [water (FA 0.1%)/acetonitrile] to afford the title compound (26.9 mg, 46% yield) as a yellow solid; LCMS (ESI, M+1): m/z=706.3.

Step B. 7-(7-(1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: A mixture of 7-(8-fluoro-2-((hexahydro-1H-pyrrolizin-7a-yl)methoxy)-7-(1-(tetrahydro-2H-pyran-2-yl)-1H-benzo[f]indazol-4-yl)pyrido[4,3-d]pyrimidin-4-yl)-1,3,7-triazaspiro[4.5]decane-2,4-dione (25.0 mg, 1.0 equiv) in TFA (962 mg, 238 equiv) was stirred at 20° C. for 0.5 hour. The mixture was concentrated and purified by prep-HPLC (column: Welch Xtimate C18 150*25 mm*5 um; mobile phase: [water (TFA)-ACN]; B %: 12%-42%, 8 min) and then further repurified by prep-HPLC (column: Welch Xtimate C18 150*25 mm*5 um; mobile phase: [water (TFA)-ACN]; B %: 10%-40%, 10 min) to afford the title compound (4.01 mg, 14% yield, TFA) as a yellow solid; ¹H NMR (400 MHZ, METHANOL-d₄) δ=9.35 (s, 1H), 8.25 (s, 1H), 8.15-8.05 (m, 2H), 7.83 (br d, J=8.8 Hz, 1H), 7.56-7.49 (m, 1H), 7.42-7.36 (m, 1H), 4.93-4.90 (m, 1H), 4.86 (br s, 1H), 4.75-4.64 (m, 3H), 4.59-4.51 (m, 1H), 4.04-3.89 (m, 2H), 3.80-3.66 (m, 2H), 2.41-2.00 (m, 12H), LCMS (ESI, M+1): m/z=622.3.

### Example 756

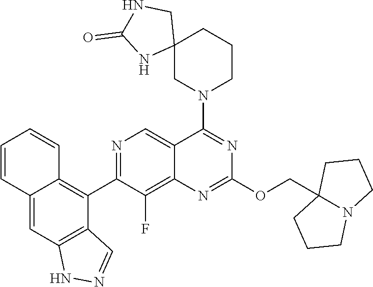 [SMILES: O=C1NCC2(CCCN(c3nc(OCC45CCCN4CCC5)nc4c(F)c(-c5c6ccccc6cc6[nH]ncc56)ncc34)C2)N1]

### 7-(7-(1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decan-2-one

The title compound was synthesized from Intermediate 21 according to the 2-step procedure described for example 755 to produce the desired compound as a yellow solid (TFA salt). ¹H NMR (400 MHZ, METHANOL-d4) δ=9.33 (s, 1H), 8.22 (s, 1H), 8.09 (d, J=8.8 Hz, 1H), 8.05-8.02 (m, 1H), 7.80 (br d, J=9.2 Hz, 1H), 7.52-7.47 (m, 1H), 7.39-7.34 (m, 1H), 4.71-4.61 (m, 2H), 4.49-4.28 (m, 2H), 4.10-3.98 (m, 1H), 3.96-3.88 (m, 1H), 3.77-3.68 (m, 2H), 3.50-3.43 (m, 1H), 3.34 (br s, 2H), 3.29 (br s, 1H), 2.40-2.29 (m, 2H), 2.28-2.17 (m, 4H), 2.16-2.08 (m, 2H), 2.07-1.87 (m, 4H); LCMS (ESI, M+1): m/z=608.3.

### Example 757

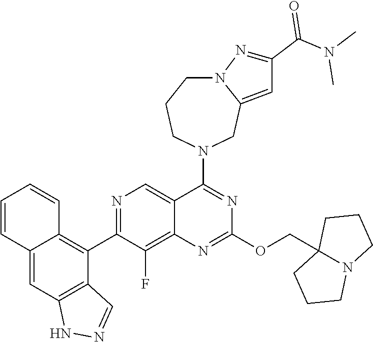 [SMILES: CN(C)C(=O)c1cc2n(n1)CCCN(c1nc(OCC34CCCN3CCC4)nc3c(F)c(-c4c5ccccc5cc5[nH]ncc45)ncc13)C2]

### 5-(7-(1H-benzo[f]indazol-4-yl)-8-fluoro-2-((hexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from Intermediate 21 according to the 2-step procedure described for example 755 to produce the desired compound as a as yellow solid (0.74 formic acid salt); ¹H NMR (400 MHZ, METHANOL-d₄) δ=9.36 (s, 1H), 8.21 (s, 1H), 8.10-8.03 (m, 2H), 7.79 (br d, J=8.0 Hz, 1H), 7.52-7.46 (m, 1H), 7.38-7.32 (m, 1H), 6.81 (s, 1H), 5.30 (s, 2H), 4.58-4.46 (m, 6H), 3.49-3.33 (m, 5H), 3.12-2.98 (m, 5H), 2.48 (br s, 2H), 2.25-2.17 (m, 2H), 2.07 (qd, J=6.4, 17.6 Hz, 4H), 1.99-1.90 (m, 2H); LCMS (ESI, M+1): m/z=661.4.

### Example 758

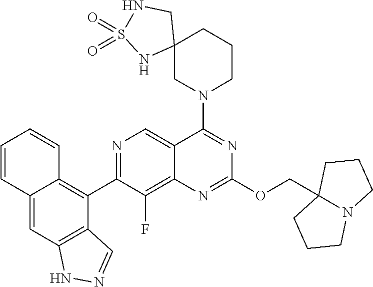 [SMILES: O=S1(=O)NCC2(CCCN(c3nc(OCC45CCCN4CCC5)nc4c(F)c(-c5c6ccccc6cc6[nH]ncc56)ncc34)C2)N1]

### 7-(7-(1H-benzo[f]indazol-4-yl)-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide

The title compound was synthesized from Intermediate 21 according to the 2-step procedure described for example 755 to produce the desired compound as a yellow solid (0.6 formic acid salt); ¹H NMR (400 MHZ, METHANOL-d₄) δ=9.31 (s, 1H), 8.21 (s, 1H), 8.12-7.98 (m, 2H), 7.85-7.76 (m, 1H), 7.49 (t, J=7.6 Hz, 1H), 7.41-7.29 (m, 1H), 4.68 (br d, J=12.8 Hz, 2H), 4.65-4.49 (m, 2H), 3.92-3.81 (m, 1H), 3.81-3.65 (m, 3H), 3.51-3.38 (m, 1H), 3.23 (br d, J=11.6 Hz, 3H), 2.44-2.30 (m, 2H), 2.29-2.16 (m, 4H), 2.16-2.04 (m, 4H), 2.03 (br s, 1H), 1.93-1.85 (m, 1H); LCMS (ESI, M+1): m/z=644.2.

### Example 759

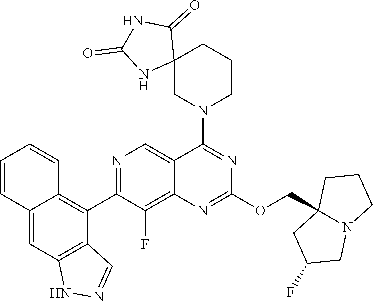 [SMILES: O=C1NC(=O)C2(CCCN(c3nc(OC[C@@]45CCCN4C[C@H](F)C5)nc4c(F)c(-c5c6ccccc6cc6[nH]ncc56)ncc34)C2)N1]

### 7-(7-(1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decane-2,4-dione

The title compound was synthesized from Intermediate 20 according to the 2-step procedure described for example 755 to produce the desired compound as a yellow solid (0.993 FA). ¹H NMR (400 MHZ, METHANOL-d₄) δ=9.31 (s, 1H), 8.43 (s, 1H), 8.22 (s, 1H), 8.13-8.01 (m, 2H), 7.80 (br d, J=9.6 Hz, 1H), 7.54-7.46 (m, 1H), 7.40-7.31 (m, 1H), 5.59-5.38 (m, 1H), 4.73-4.58 (m, 2H), 4.56-4.47 (m, 2H), 3.95-3.81 (m, 2H), 3.80-3.67 (m, 1H), 3.67-3.55 (m, 2H), 3.26 (br s, 1H), 2.64-2.41 (m, 2H), 2.38-2.25 (m, 2H), 2.25 (br s, 2H), 2.17-2.03 (m, 3H), 2.02-1.93 (m, 1H); LCMS (ESI, M+1): m/z=640.4.

### Example 760

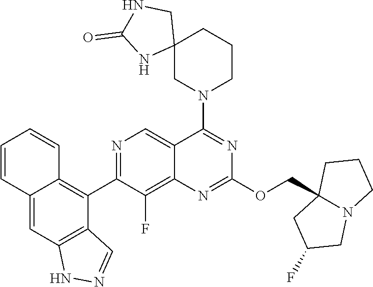 [SMILES: O=C1NCC2(CCCN(c3nc(OC[C@@]45CCCN4C[C@H](F)C5)nc4c(F)c(-c5c6ccccc6cc6[nH]ncc56)ncc34)C2)N1]

### 7-(7-(1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decan-2-one

The title compound was synthesized from Intermediate 20 according to the 2-step procedure described for example 755 to produce the desired compound as a yellow solid; ¹H NMR (400 MHZ, METHANOL-d₄) δ=9.27 (s, 1H), 8.20 (s, 1H), 8.14-7.97 (m, 2H), 7.79 (br d, J=9.2 Hz, 1H), 7.53-7.45 (m, 1H), 7.40-7.32 (m, 1H), 5.46-5.17 (m, 1H), 4.39-4.24 (m, 2H), 4.18-3.98 (m, 4H), 3.52-3.36 (m, 2H), 3.25-3.18 (m, 3H), 3.04-2.97 (m, 1H), 2.35 (br t, J=4.8 Hz, 3H), 2.08-1.87 (m, 7H) LCMS (ESI, M+1): m/z=626.3

### Example 761

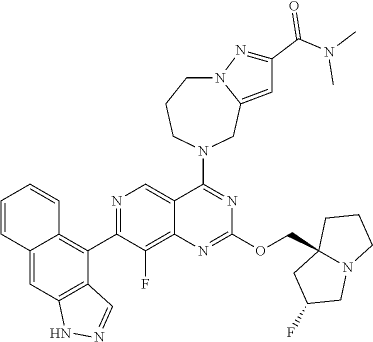 [SMILES: CN(C)C(=O)c1cc2n(n1)CCCN(c1nc(OC[C@@]34CCCN3C[C@H](F)C4)nc3c(F)c(-c4c5ccccc5cc5[nH]ncc45)ncc13)C2]

### 5-(7-(1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

The title compound was synthesized from Intermediate 20 according to the 2-step 31 procedure described for example 755 to produce the desired compound as a yellow solid (0. formic acid salt); ¹H NMR (400 MHZ, METHANOL-d₄) δ=9.34 (s, 1H), 8.20 (s, 1H), 8.10-8.03 (m, 2H), 7.79 (br d, J=8.8 Hz, 1H), 7.51-7.46 (m, 1H), 7.38-7.32 (m, 1H), 6.80 (s, 1H), 5.47-5.31 (m, 1H), 5.29 (s, 2H), 4.58-4.52 (m, 2H), 4.49 (br t, J=5.2 Hz, 2H), 4.39 (q, J=11.2 Hz, 2H), 3.55-3.37 (m, 3H), 3.35 (s, 3H), 3.14 (dt, J=5.8, 9.6 Hz, 1H), 3.08 (s, 3H), 2.50-2.28 (m, 4H), 2.23-1.95 (m, 4H); LCMS (ESI, M+1): m/z=679.4;

### Example 762

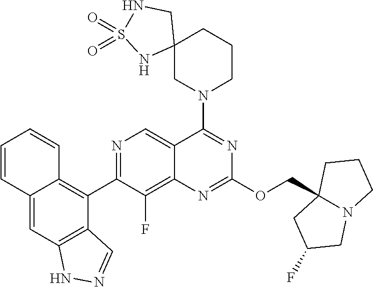 [SMILES: O=S1(=O)NCC2(CCCN(c3nc(OC[C@@]45CCCN4C[C@H](F)C5)nc4c(F)c(-c5c6ccccc6cc6[nH]ncc56)ncc34)C2)N1]

### 7-(7-(1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,7-triazaspiro[4.5]decane 2,2-dioxide

The title compound was synthesized from Intermediate 20 according to the 2-step procedure described for example 755 to produce the desired compound as a white solid (0.64 formic acid salt); ¹H NMR (400 MHZ, METHANOL-d₄) δ=9.29-9.23 (m, 1H), 8.20 (s, 1H), 8.09-8.02 (m, 2H), 7.84-7.77 (m, 1H), 7.51-7.45 (m, 1H), 7.39-7.30 (m, 1H), 5.50-5.25 (m, 1H), 4.67-4.62 (m, 1H), 4.56-4.49 (m, 2H), 4.42 (br d, J=10.8 Hz, 1H), 3.90-3.79 (m, 1H), 3.76-3.64 (m, 1H), 3.62-3.51 (m, 1H), 3.46 (br s, 1H), 3.41 (br d, J=11.6 Hz, 2H), 3.23-3.17 (m, 2H), 2.54-2.40 (m, 1H), 2.40-2.32 (m, 1H), 2.30-2.21 (m, 1H), 2.17-2.07 (m, 3H), 2.06-1.95 (m, 3H), 1.91-1.82 (m, 1H); LCMS (ESI, M+1): m/z=662.3

### Example 763

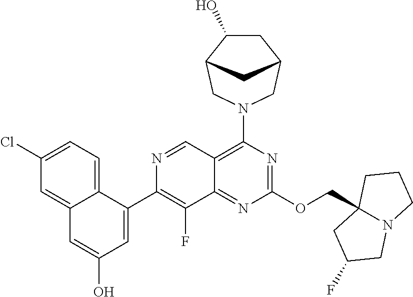 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccc(Cl)cc2c1]

### (1R,5R,6R)-3-(7-(6-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

The title compound was synthesized from 2-(3-chlorophenyl) acetic acid according to the 8-step procedure described for example 746 to produce the desired compound as a white solid (0.40 formic acid salt); ¹H NMR (400 MHZ, DMSO-d₆) δ=9.39 (s, 1H), 8.22 (s, 1H), 7.94 (d, J=2.0 Hz, 1H), 7.56 (br d, J=8.4 Hz, 1H), 7.29 (d, J=2.0 Hz, 1H), 7.28-7.22 (m, 2H), 5.42-5.15 (m, 1H), 4.88-4.51 (m, 3H), 4.22-4.08 (m, 2H), 4.03 (dd, J=4.0, 10.4 Hz, 1H), 3.71 (br d, J=12.0 Hz, 1H), 3.35 (br d, J=12.8 Hz, 1H), 3.12-2.99 (m, 3H), 2.86-2.79 (m, 1H), 2.33 (br d, J=2.0 Hz, 1H), 2.23-1.93 (m, 5H), 1.93-1.58 (m, 5H), 1.25 (br d, J=13.2 Hz, 1H); LCMS (ESI, M+1): m/z=608.2.

### Example 764

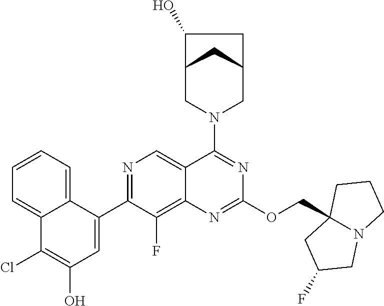 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1Cl]

### (1R,5R,6R)-3-(7-(4-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

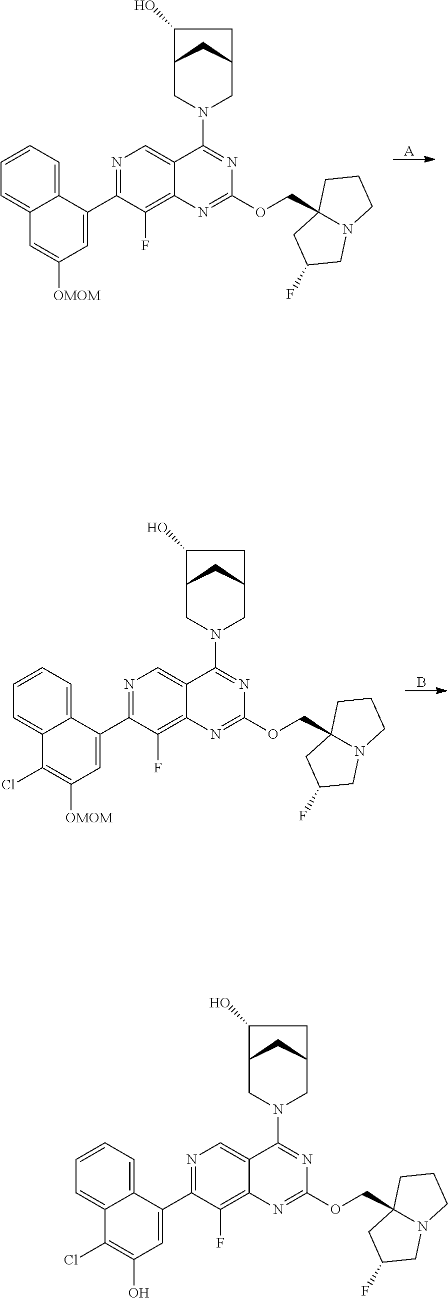

Step A. (1R,5R,6R)-3-(7-(4-chloro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: A mixture of (1R,5R,6R)-3-(8-fluoro-2-(((2R,7aS)-2-fluorotetrahydro-1H-pyrrolizin-7a(5H)-yl)methoxy)-7-(3-(methoxymethoxy)naphthalen-1-yl)pyrido[4,3-d]pyrimidin-4-yl)-3-azabicyclo[3.2.1]octan-6-ol (100 mg, 1.00 eq.) and NCS (28.1 mg, 1.30 eq.) in ACN (1.50 mL) and DMF (0.20 mL) was stirred at 70° C. for 12 hours. The reaction mixture was filtered, concentrated and purified with prep-HPLC (column: 3_Phenomenex Luna C18 75*30 mm*3 um; mobile phase: [water (TFA)-ACN]; B %: 34%-54%, 9 min) to afford title compound (40.0 mg, 36% yield) as a yellow solid; ¹H NMR (400 MHZ, DMSO-d6) δ=10.83-10.67 (m, 1H), 9.48 (s, 1H), 8.28 (d, J=8.4 Hz, 1H), 7.79-7.65 (m, 3H), 7.57-7.48 (m, 1H), 5.65-5.50 (m, 1H), 5.46 (s, 2H), 4.84-4.80 (m, 1H), 4.63 (br d, J=12.4 Hz, 1H), 4.59 (br d, J=2.4 Hz, 2H), 4.18 (td, J=5.2, 10.4 Hz, 1H), 3.92-3.69 (m, 5H), 3.48 (s, 3H), 3.39 (br d, J=13.2 Hz, 1H), 3.35-3.28 (m, 1H), 2.57 (br d, J=5.6 Hz, 1H), 2.35-2.31 (m, 2H), 2.21-2.05 (m, 5H), 1.80 (br d, J=12.0 Hz, 1H), 1.68 (br dd, J=4.4, 7.2 Hz, 1H), 1.27-1.20 (m, 1H). LCMS (ESI, M+1): m/z=652.4.

Step B. (1R,5R,6R)-3-(7-(4-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: A solution of (1R,5R,6R)-3-(7-(4-chloro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (30.0 mg, 46.0 μmol, 1.00 eq.) in HCl/MeOH (4 M, 3.00 mL, 261 eq.) was stirred at 0° C. for 1 hour. The reaction mixture was concentrated and purified with prep-HPLC (column: Phenomenex luna C18 150*25 mm*10 um; mobile phase: [water (FA)-ACN]; B %: 14%-44%, 10 min) to afford title compound (7.81 mg, 28% yield) as a white solid; ¹H NMR (400 MHZ, DMSO-d6) δ=10.74 (s, 1H), 9.41 (s, 1H), 8.16 (d, J=8.4 Hz, 1H), 7.71-7.57 (m, 2H), 7.44 (s, 1H), 7.42-7.35 (m, 1H), 5.44-5.17 (m, 1H), 4.80-4.71 (m, 2H), 4.61 (br d, J=12.4 Hz, 1H), 4.15 (br dd, J=5.4, 8.8 Hz, 2H), 4.10-4.00 (m, 1H), 3.72 (br d, J=11.2 Hz, 1H), 3.38 (br s, 1H), 3.21-2.97 (m, 3H), 2.90-2.80 (m, 1H), 2.22-1.94 (m, 6H), 1.89-1.73 (m, 4H), 1.71-1.61 (m, 1H), 1.25 (br d, J=13.6 Hz, 1H). LCMS (ESI, M+1): m/z=608.3.

### Example 765

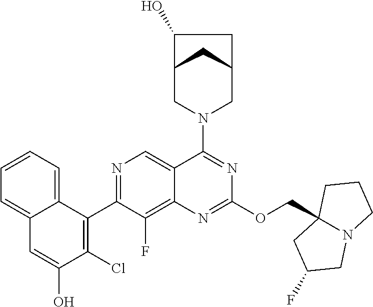 [SMILES: Oc1cc2ccccc2c(-c2ncc3c(N4C[C@@H]5C[C@H](C4)[C@H](O)C5)nc(OC[C@@]45CCCN4C[C@H](F)C5)nc3c2F)c1Cl]

### (1R,5R,6R)-3-(7-(2-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

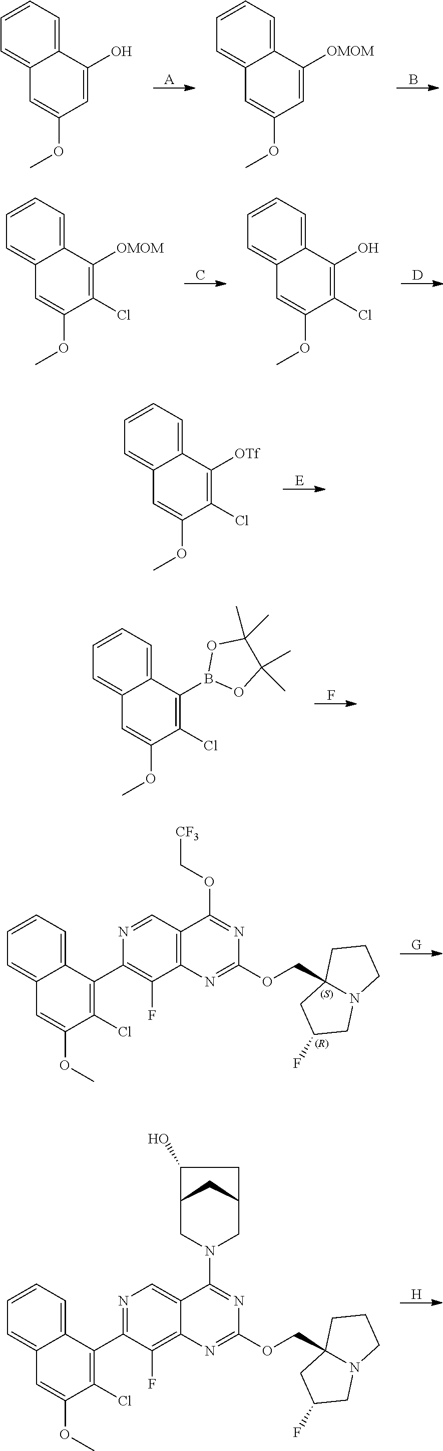

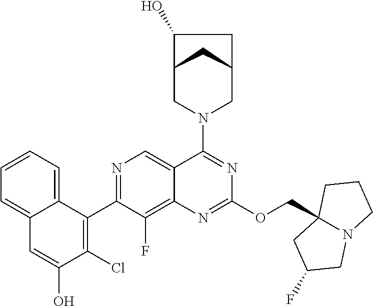 [SMILES: Oc1cc2ccccc2c(-c2ncc3c(N4C[C@@H]5C[C@H](C4)[C@H](O)C5)nc(OC[C@@]45CCCN4C[C@H](F)C5)nc3c2F)c1Cl]

Step A. 3-methoxy-1-(methoxymethoxy)naphthalene: To a solution of 3-methoxynaphthalen-1-ol (10.0 g, 1.0 equiv) in THF (100 mL) was added DIPEA (22.3 g, 3.0 equiv) and bromo(methoxy) methane (8.61 g, 1.2 equiv), the mixture was stirred at 25° C. for 0.5 hour. The reaction mixture was diluted with EtOAc (100 mL) and water (200 mL), extracted with EtOAc (100 mL), the combined organic phase was washed with brine (50 mL), dried over anhydrous sodium sulfate, filtered and concentrated to give a residue. The residue was purified by column chromatography (SiO₂, petroleum ether/ethyl acetate 15:1 to 10:1) to afford the title compound (10.5 g, 82% yield) as a yellow oil; ¹H NMR (400 MHZ, CHLOROFORM-d) δ=8.08 (d, J=8.4 Hz, 1H), 7.63-7.58 (m, 1H), 7.36 (t, J=7.6 Hz, 1H), 7.28-7.21 (m, 1H), 6.72 (s, 2H), 5.28 (s, 2H), 3.82 (s, 3H), 3.46 (s, 3H); LCMS (ESI, M+1): m/z=219.2.

Step B. 2-chloro-3-methoxy-1-(methoxymethoxy)naphthalene: To a solution of 3-methoxy-1-(methoxymethoxy)naphthalene (7.50 g, 1.0 equiv) in THF (75 mL) was slowly added n-BuLi (2.5 M, 27.5 mL, 2.0 equiv) and tetramethylethylenediamine (4.04 g, 1.0 equiv) under nitrogen at 0° C. and the mixture was stirred at 0° C. for 2 hours. Then a solution of 1,1,1,2,2,2-hexachloroethane (16.3 g, 2.0 equiv) in THF (10 mL) was added to the above reaction and the mixture was stirred at 20° C. for 0.5 hour. The mixture was slowly quenched with water (300 mL) and extracted with ethyl acetate (3×200 mL). The combined organic layer was dried over anhydrous sodium sulfate, and concentrated under vacuum to give a residue. The residue was purified by column chromatography (SiO₂, petroleum ether/ethyl acetate 30:1 to 10:1) to afford the title compound (8.00 g, 92% yield) as a yellow oil; ¹H NMR (400 MHZ, CHLOROFORM-d) δ=8.10 (dd, J=0.6, 8.0 Hz, 1H), 7.76-7.71 (m, 1H), 7.51-7.39 (m, 2H), 7.04 (s, 1H), 5.31 (s, 2H), 4.05-3.98 (m, 3H), 3.76-3.68 (m, 3H).

Step C. 2-chloro-3-methoxynaphthalen-1-ol: To a solution of 2-chloro-3-methoxy-1-(methoxymethoxy)naphthalene (4.00 g, 1.0 equiv) in DCM (20 mL) was added TFA (30.8 g, 17 equiv). The reaction mixture was stirred at 0° C. for 0.5 hour. The reaction mixture was diluted with water (100 mL), extracted with EtOAc (2×50 mL), the combined organic phase was washed with brine (100 mL), dried over anhydrous sodium sulfate, filtered and concentrated to give a residue. The residue was purified by reversed phase flash chromatography [C18, 0.1% formic acid] to afford the title compound (1.80 g, 54% yield) as a yellow oil; ¹H NMR (400 MHZ, CHLOROFORM-d) δ=8.17-8.10 (m, 1H), 7.71-7.66 (m, 1H), 7.47 (t, J=7.2 Hz, 1H), 7.41-7.35 (m, 1H), 6.82 (s, 1H), 6.16 (br s, 1H), 4.00 (s, 3H).

Step D. 2-chloro-3-methoxynaphthalen-1-yl trifluoromethanesulfonate: To a solution of 2-chloro-3-methoxynaphthalen-1-ol (1.00 g, 1.0 equiv) in DCM (10 mL) at 0° C. was added TEA (970 mg, 2.0 equiv) and Tf₂O (2.03 g, 1.5 equiv). The mixture was stirred at 0° C. for 0.5 hour. The reaction mixture was diluted with water (40 mL), extracted with EtOAc (2×20 mL), the combined organic phase was washed with brine (20 mL), dried over anhydrous sodium sulfate, filtered and concentrated to give a residue. The residue was purified by column chromatography (SiO₂, petroleum ether/ethyl acetate 50:1 to 30:1) to afford the title compound (1.33 mg, 81% yield) as a yellow solid; ¹H NMR (400 MHZ, CHLOROFORM-d) δ=8.00 (d, J=8.4 Hz, 1H), 7.83-7.77 (m, 1H), 7.61-7.50 (m, 2H), 7.25 (s, 1H), 4.05 (s, 3H).

Step E. 2-(2-chloro-3-methoxynaphthalen-1-yl)-4,4,5,5-tetramethyl-1,3,2-dioxaborolane: A mixture of (2-chloro-3-methoxy-1-naphthyl)trifluoromethanesulfonate (1.33 g, 1.0 equiv), bis(pinacolato)diboron (3.97 g, 4.0 equiv), KOAc (1.15 g, 3.0 equiv), and Pd(dppf)Cl₂ (286 mg, 0.1 equiv) in dioxane (13 mL) was degassed and stirred at 100° C. for 16 hours under N₂ atmosphere. The reaction mixture was diluted with water (40 mL), extracted with EtOAc (2×30 mL), the combined organic phase was washed with brine (50 mL), dried over anhydrous sodium sulfate, filtered and concentrated to give a residue. The residue was purified by column chromatography (SiO₂, petroleum ether/ethyl acetate 20:1 to 5:1) to afford the title compound (590 mg, 47% yield) as a white solid; ¹H NMR (400 MHZ, CHLOROFORM-d) δ=7.79 (d, J=8.0 Hz, 1H), 7.71 (d, J=8.0 Hz, 1H), 7.44-7.36 (m, 1H), 7.39-7.33 (m, 1H), 7.19 (s, 1H), 3.99 (s, 3H), 1.52 (s, 12H).

Step F. 7-(2-chloro-3-methoxynaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: A mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (400 mg, 1.0 equiv), 2-(2-chloro-3-methoxy-1-naphthyl)-4,4,5,5-tetramethyl-1,3,2-dioxaborolane (377 mg, 1.3 equiv), CataCXium A Pd G3 (66.4 mg, 0.1 equiv), and Cs₂CO₃ (1.5 M, 3.0 equiv) in methoxycyclopentane (10 mL) was degassed and purged with N₂ for 3 times, and then the mixture was stirred at 80° C. for 6 hours under N₂ atmosphere. The reaction mixture was diluted with water (40 mL), extracted with EtOAc (2×30 mL), the combined organic phase was washed with brine (60 mL), dried over anhydrous sodium sulfate, filtered and concentrated to give a residue. The residue was purified by reversed phase flash chromatography [C18, 0.1% formic acid condition] to afford the title compound (289 mg, 53% yield) as a yellow solid; LCMS (ESI, M+1): m/z=595.2.

Step G. (1R,5R,6R)-3-(7-(2-chloro-3-methoxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a mixture of 7-(2-chloro-3-methoxynaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (280 mg, 1.0 equiv) in DMF (3 mL) was added (1R,5R,6R)-3-azabicyclo[3.2.1]octan-6-ol (120 mg, 2.0 equiv), DIPEA (182 mg, 3.0 equiv) and 4 Å molecular sieves (20 mg). The reaction mixture was stirred at 40° C. for 16 hours. The mixture was filtered and the filtrate was purified by reversed phase flash chromatography [C18, 0.1% formic acid condition] to afford the title compound (240 mg, 82% yield) as a yellow solid; LCMS (ESI, M+1): m/z=622.3.

Step H. (1R,5R,6R)-3-(7-(2-chloro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of (1R,5R,6R)-3-(7-(2-chloro-3-methoxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (40.0 mg, 1.0 equiv) in DCM (1 mL) was added a solution of BBr₃ (80.5 mg, 5.0 equiv) in DCM (0.5 mL) dropwise at 0° C. The resulting mixture was stirred at 0° C. for 5 hours. The mixture was poured into ice-cooled saturated NaHCO₃ solution (10 mL), and extracted with EtOAc (2×5 mL), the combined organic phase was washed with brine (10 mL), dried over anhydrous Na₂SO₄, filtered, concentrated and purified by prep-HPLC [column: Waters Xbridge 150×25 mm×5 μm; Phase A: water (10 mM NH₄HCO₃), phase B: ACN, B %: 28%-58% over 8 min] to afford the title compound (7.10 mg, 17% yield) as a white solid; ¹H NMR (400 MHZ, METHANOL-d4) δ=9.33 (s, 1H), 7.76 (d, J=8.0 Hz, 1H), 7.47-7.39 (m, 1H), 7.27-7.20 (m, 1H), 7.19-7.14 (m, 1H), 5.41-5.22 (m, 1H), 5.00-4.92 (m, 2H), 4.39-4.28 (m, 2H), 4.28-4.19 (m, 1H), 3.83-3.75 (m, 1H), 3.49 (br d, J=12.4 Hz, 1H), 3.28-3.19 (m, 3H), 3.07-2.97 (m, 1H), 2.43-2.40 (m, 1H), 2.36-2.11 (m, 5H), 2.07-1.88 (m, 4H), 1.86-1.79 (m, 1H), 1.46-1.37 (m, 1H); LCMS (ESI, M+1): m/z=608.4.

### Example 766

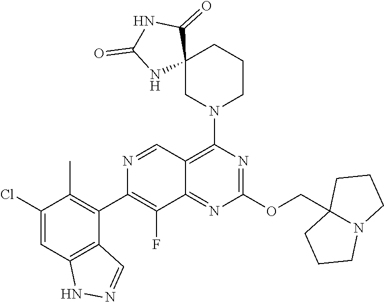 [SMILES: Cc1c(Cl)cc2[nH]ncc2c1-c1ncc2c(N3CCC[C@]4(C3)NC(=O)NC4=O)nc(OCC34CCCN3CCC4)nc2c1F]

### (5R)-7-(7-(6-chloro-5-methyl-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione

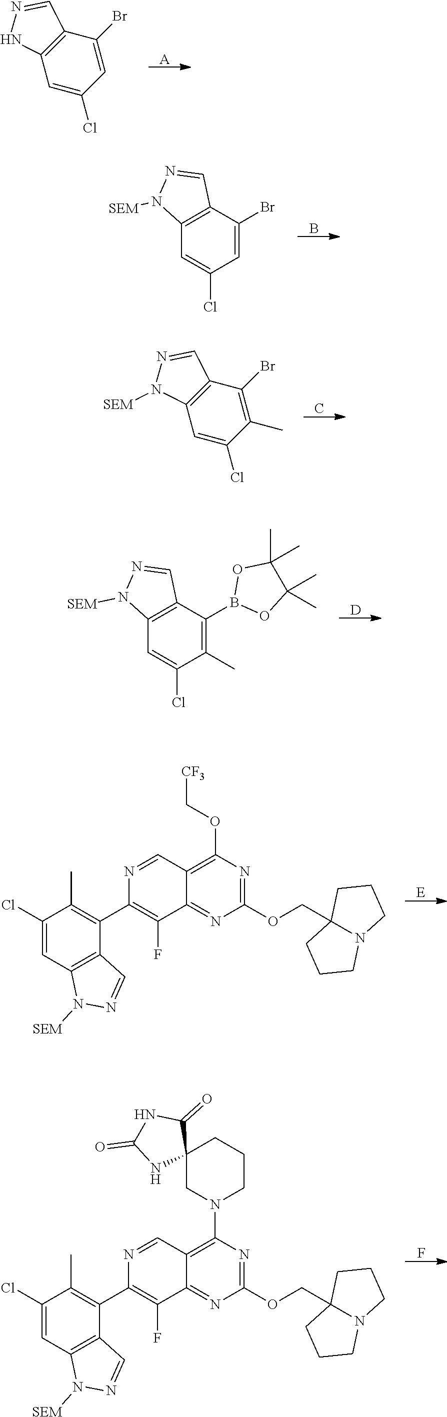

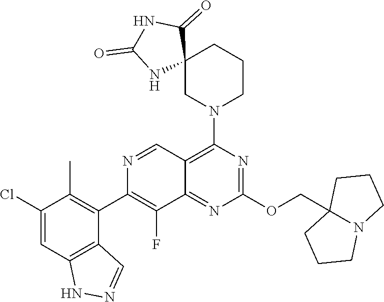 [SMILES: Cc1c(Cl)cc2[nH]ncc2c1-c1ncc2c(N3CCC[C@]4(C3)NC(=O)NC4=O)nc(OCC34CCCN3CCC4)nc2c1F]

Step A. 4-bromo-6-chloro-1-((2-(trimethylsilyl)ethoxy)methyl)-1H-indazole: To a solution of 4-bromo-6-chloro-1H-indazole (15.0 g, 1.0 equiv) in THF (200 mL) was added NaH (3.89 g, 60% purity, 1.5 equiv) at 0° C. The mixture was stirred at 20° C. for 0.5 hour. To the mixture was added SEM-Cl (13.0 g, 1.2 equiv) at 0° C. The mixture was stirred at 20° C. for 2 hours. The mixture was quenched with H₂O (100 mL) and extracted with EtOAc (3×100 mL). The combined organic layers were washed with brine (100 mL), dried over anhydrous sodium sulfate, concentrated and purified by column chromatography [SiO₂, Petroleum ether/Ethyl acetate 50:1 to 10:1] to afford the tittle compound (15.0 g, 64% yield) as a yellow solid; ¹H NMR (400 MHz, CHLOROFORM-d) δ=7.99 (d J=0.8 Hz, 1H), 7.55 (d, J=0.8 Hz, 1H), 7.36 (d, J=1.6 Hz, 1H), 5.69 (s, 2H), 3.57-3.50 (m, 2H), 0.93-0.86 (m, 2H), −0.05 (s, 9H).

Step B. 4-bromo-6-chloro-5-methyl-1-((2-(trimethylsilyl)ethoxy)methyl)-1H-indazole: To a solution of 2-[(4-bromo-6-chloro-indazol-1-yl)methoxy]ethyl-trimethyl-silane (2.0 g, 1.0 equiv) in THF (20 mL) was added LDA (2.0 M, 4.15 mL, 1.5 equiv) at −65° C. under N₂. The mixture was stirred at −65° C. for 1 hour. To the solution was slowly added MeI (3.92 g, 5 equiv) at −65° C. The mixture was stirred at 25° C. for 0.5 hour under N₂. The mixture was quenched with H₂O (20 mL) at 0° C. The mixture was extracted with EtOAc (3×20 mL). The combined organic layers were washed with brine (50 mL), dried over anhydrous sodium sulfate, concentrated and purified by reversed phase flash chromatography [C18, 0.1% formic acid condition] to afford the title compound (1.10 g, 52% yield) as a yellow solid; ¹H NMR (400 MHZ, CHLOROFORM-d) δ=7.99 (s, 1H), 7.43 (s, 1H), 5.88 (s, 2H), 3.65-3.53 (m, 2H), 2.81 (s, 3H), 0.99-0.88 (m, 2H), 0.00 (s, 9H).

Step C. 6-chloro-5-methyl-4-(4,4,5,5-tetramethyl-1,3,2-dioxaborolan-2-yl)-1-((2-(trimethylsilyl)ethoxy)methyl)-1H-indazole: To a solution of 4-bromo-6-chloro-5-methyl-1-((2-(trimethylsilyl)ethoxy)methyl)-1H-indazole (5.0 g, 1.0 equiv) and Pin₂B₂ (5.07 g, 1.5 equiv) in toluene (100 mL) were added Pd(dppf)Cl₂ (974 mg, 0.1 equiv) and KOAc (3.92 mg, 3.0 equiv) under N₂. The mixture was stirred at 110° C. for 12 hours. The reaction mixture was diluted with H₂O (100 mL) and extracted with EtOAc (3×50 mL). The combined organic layers were washed with brine (100 mL), dried over anhydrous sodium sulfate, concentrated and purified by reversed phase flash chromatography [C18, 0.1% formic acid condition] to afford the title compound (3.0 g, 52% yield) as a yellow liquid; ¹H NMR (400 MHZ, CHLOROFORM-d) δ=8.32 (s, 1H), 7.68 (s, 1H), 5.85 (s, 2H), 3.56-3.51 (m, 2H), 2.85 (s, 3H), 1.40 (s, 12H), 0.90-0.86 (m, 2H), −0.05 (s, 9H); LCMS (ESI, M−1): m/z=423.1.

Step D. 7-(6-chloro-5-methyl-1-((2-(trimethylsilyl)ethoxy)methyl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine: To a solution of 7-chloro-8-fluoro-2-((tetrahydro-1H-pyrrolizin-7a(5H)-yl)methoxy)-4-(2,2,2-trifluoroethoxy)pyrido[4,3-d]pyrimidine (800 mg, 1.0 equiv) and K₃PO₄ (1.50 M, 3.80 mL, 3.0 equiv) in toluene (8.0 mL) were added Ad₂nBup-Pd-G3 (138 mg, 0.1 equiv) and 6-chloro-5-methyl-4-(4,4,5,5-tetramethyl-1,3,2-dioxaborolan-2-yl)-1-((2-(trimethylsilyl)ethoxy)methyl)-1H-indazole (1.21 g, 1.5 equiv). The mixture was stirred at 60° C. for 12 hours under N₂. The reaction mixture was diluted with H₂O (10 mL) and extracted with EtOAc (3×20 mL). The combined organic layers were dried over anhydrous sodium sulfate, concentrated and purified by reversed phase flash chromatography [C18, 0.1% formic acid condition] to afford the title compound (300 mg, 22% yield) as a yellow solid; LCMS (ESI, M+1): m/z=681.1.

Step E. (5R)-7-(7-(6-chloro-5-methyl-1-((2-(trimethylsilyl)ethoxy)methyl)-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a solution of 7-(6-chloro-5-methyl-1-((2-(trimethylsilyl)ethoxy)methyl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine (300 mg, 1.0 equiv) in DMF (1 mL) were added DIPEA (171 mg, 3.0 equiv) and (R)-1,3,7-triazaspiro[4.5]decane-2,4-dione (223 mg, 3.0 equiv). The mixture was stirred at 40° C. for 6 hours. The reaction mixture was concentrated and purified by reversed phase flash chromatography [C18, 0.1% formic acid condition] to afford the title compound (200 mg, 58% yield) as an off-white solid; LCMS (ESI, M+1): m/z=750.2.

Step F. (5R)-7-(7-(6-chloro-5-methyl-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a solution of (5R)-7-(7-(6-chloro-5-methyl-1-((2-(trimethylsilyl)ethoxy)methyl)-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione (100 mg, 1.0 equiv) in DCM (2 mL) was added TFA (3.08 g, 203 equiv). The mixture was stirred at 20° C. for 0.5 hour. The reaction mixture was concentrated and purified with prep-HPLC [Phenomenex C18 75×30 mm×3 μm; A: water (10 mM FA), B: ACN, B %: 10%-40% over 7 min] and lyophilized to afford the title compound (135 mg, 75% yield, 0.61 FA) as a white solid; ¹H NMR (400 MHZ, METHANOL-d₄) δ=9.24-9.19 (m, 1H), 8.52 (s, 1H), 8.37 (d, J=1.2 Hz, 1H), 7.71 (s, 1H), 4.67-4.61 (m, 1H), 4.60-4.53 (m, 2H), 4.50-4.41 (m, 1H), 3.88-3.74 (m, 2H), 3.67-3.58 (m, 2H), 3.26-3.15 (m, 2H), 2.68 (s, 3H), 2.35-2.24 (m, 3H), 2.23-2.10 (m, 5H), 2.09-2.01 (m, 3H), 1.99-1.94 (m, 1H); LCMS (ESI, M+1): m/z=620.2.

### Example 767

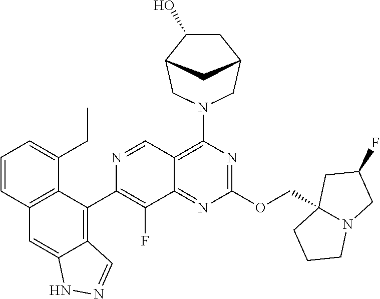 [SMILES: CCc1cccc2cc3[nH]ncc3c(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

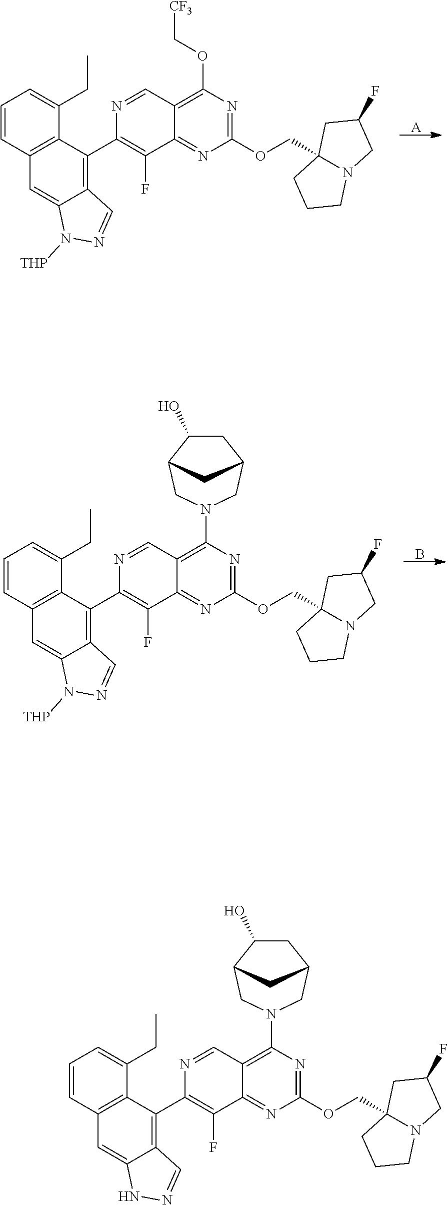

Step A. (1R,5R,6R)-3-(7-(5-ethyl-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a mixture of 5-ethyl-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole (51.0 mg, 1.0 equiv), (1R,5R,6R)-3-azabicyclo[3.2.1]octan-6-ol (14.2 mg, 1.5 equiv) and 4 Å molecular sieves (10 mg) in DMF (1 mL) was added DIPEA (29.0 mg, 3.0 equiv). The mixture was stirred at 40° C. for 10 hours. The mixture was filtered, concentrated and purified by reversed phase flash chromatography [C18, 0.1% formic acid condition] to afford the title compound (43.0 mg, 77% yield) as a yellow solid; LCMS (ESI, M+1): m/z=710.5.

Step B. (1R,5R,6R)-3-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To the solution of (1R,5R,6R)-3-(7-(5-ethyl-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (40.0 mg, 1.0 equiv) in DCM (0.5 mL) was added TFA (1.54 g, 240 equiv) at 0° C. The mixture was stirred at 15° C. for 0.5 hour. The pH of the mixture was adjusted 8 by sat. aq. NaHCO₃. The mixture was extracted with DCM (2×10 mL). The organic layer was dried over Na₂SO₄, concentrated and purified by prep-HPLC [column: YMC Triart C18 150×25 mm×5 μm; A: water (FA), B: ACN, B %: 20%-50% over 10 min] to afford the title compound (6.53 mg, 0.15 FA, 18% yield) as a yellow solid; ¹H NMR (400 MHZ, METHANOL-d₄) δ=9.34 (d, J=11.6 Hz, 1H), 8.20 (s, 1H), 7.94 (br d, J=8.4 Hz, 1H), 7.77-7.70 (m, 1H), 7.41 (br t, J=7.2 Hz, 1H), 7.27 (br d, J=7.2 Hz, 1H), 5.49-5.29 (m, 1H), 4.49-4.31 (m, 3H), 3.82 (br t, J=11.2 Hz, 1H), 3.56-3.36 (m, 4H), 3.21-3.09 (m, 1H), 2.52-2.17 (m, 8H), 2.14-2.06 (m, 2H), 2.00-1.76 (m, 4H), 1.48-1.17 (m, 2H), 1.03-0.91 (m, 3H); LCMS (ESI, M+1): m/z=626.4.

### Example 768

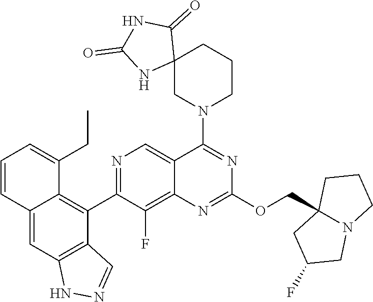 [SMILES: CCc1cccc2cc3[nH]ncc3c(-c3ncc4c(N5CCCC6(C5)NC(=O)NC6=O)nc(OC[C@@]56CCCN5C[C@H](F)C6)nc4c3F)c12]

### 7-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decane-2,4-dione

The title compound was synthesized from Intermediate 22 according to the 2-step procedure described for example 767 to produce the desired compound as a yellow solid; ¹H NMR (400 MHZ, METHANOL-d₄) δ=9.22 (s, 1H), 8.20 (s, 1H), 7.95 (d, J=8.4 Hz, 1H), 7.76-7.67 (m, 1H), 7.45-7.38 (m, 1H), 7.30-7.24 (m, 1H), 5.38-5.23 (m, 1H), 4.71-4.45 (m, 2H), 4.43 (br s, 2H), 3.87-3.67 (m, 2H), 3.30-3.15 (m, 3H), 3.07-2.96 (m, 1H), 2.54-2.35 (m, 2H), 2.34-2.11 (m, 4H), 2.06-1.88 (m, 6H), 1.04-0.89 (m, 3H); LCMS (ESI, M+1): m/z=668.3.

### Example 769

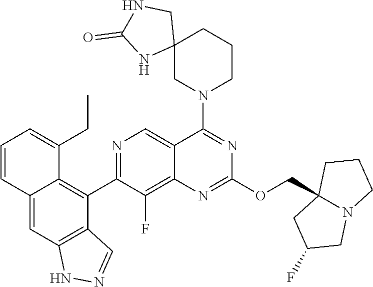 [SMILES: CCc1cccc2cc3[nH]ncc3c(-c3ncc4c(N5CCCC6(CNC(=O)N6)C5)nc(OC[C@@]56CCCN5C[C@H](F)C6)nc4c3F)c12]

### 7-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decan-2-one

The title compound was synthesized from Intermediate 22 according to the 2-step procedure described for example 767 to produce the desired compound as a yellow solid (0.29 formic acid salt); ¹H NMR (400 MHZ, METHANOL-d₄) δ=9.23 (s, 1H), 8.20 (s, 1H), 7.95 (br d, J=8.6 Hz, 1H), 7.71 (br d, J=4.8 Hz, 1H), 7.41 (br t, J=7.6 Hz, 1H), 7.28 (br d, J=2.4 Hz, 1H), 5.49-5.26 (m, 1H), 4.53-4.10 (m, 4H), 4.05-3.85 (m, 2H), 3.62-3.34 (m, 5H), 3.14 (br d, J=5.4 Hz, 1H), 2.60-2.34 (m, 3H), 2.33-2.17 (m, 2H), 2.08 (br d, J=7.6 Hz, 2H), 2.03-1.81 (m, 5H), 1.04-0.87 (m, 3H); LCMS (ESI, M+1): m/z=654.4.

### Example 770

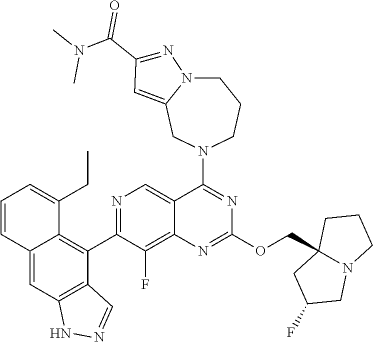 [SMILES: CCc1cccc2cc3[nH]ncc3c(-c3ncc4c(N5CCCn6nc(C(=O)N(C)C)cc6C5)nc(OC[C@@]56CCCN5C[C@H](F)C6)nc4c3F)c12]

### 5-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

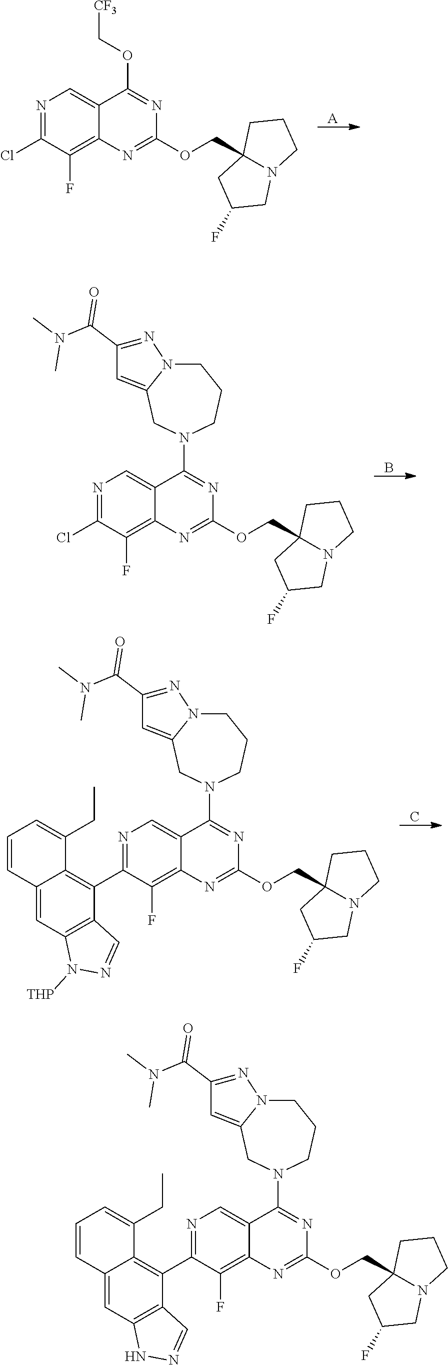

Step A. 5-(7-chloro-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (300 mg, 1.0 equiv) and N,N-dimethyl-5,6,7,8-tetrahydro-4H-pyrazolo[1,5-a][1,4]diazepine-2-carboxamide (171 mg, 1.2 equiv) in DMF (2 mL) was added DIPEA (265 mg, 3.0 equiv). The mixture was stirred at 40° C. for 10 hours. The mixture was filtered and purified by reversed phase flash chromatography [C18, 0.1% formic acid condition] to afford the title compound (180 mg, 45% yield) as a white solid; LCMS (ESI, M+1): m/z=547.3.

Step B. 5-(7-(5-ethyl-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a mixture of 5-(7-chloro-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (160 mg, 1.3 equiv), 5-ethyl-1-(tetrahydro-2H-pyran-2-yl)-4-(4,4,5,5-tetramethyl-1,3,2-dioxaborolan-2-yl)-1H-benzo[f]indazole (90.0 mg, 0.98 equiv) and K₃PO₄ (1.5 M, 0.45 mL, 3.0 equiv) in methoxycyclopentane (1.5 mL) was added CataCXium A Pd G3 (16.4 mg, 0.1 equiv) under N₂. The mixture was stirred at 90° C. for 2 hours. The mixture was diluted with water (10 mL) and extracted with ethyl acetate (10 mL). The organic layer was dried over Na₂SO₄ and concentrated to give a residue. The residue was purified by reversed phase flash chromatography [C18, 0.1% formic acid condition] to afford the title compound (100 mg, 54% yield) as a yellow solid; LCMS (ESI, M+1): m/z=791.5.

Step C. 5-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 5-(7-(5-ethyl-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (95.0 mg, 1.0 equiv) in DCM (0.5 mL) was added TFA (1.54 g, 112 equiv) at 0° C. The mixture was stirred at 15° C. for 1 hour. The mixture was quenched by sat. NaHCO₃ (50 mL) and extracted with ethyl acetate (2×10 mL). The organic layer was dried over Na₂SO₄ and concentrated to give a residue. The residue was purified by prep-HPLC [column: Phenomenex luna C18 150×25 mm×10 μm; A: water (FA), B: ACN, B %: 12%-42% over 10 min] and lyophilized to afford the title compound (12.2 mg, 14% yield) as a yellow solid; ¹H NMR (400 MHZ, METHANOL-d₄) δ=9.29 (s, 1H), 8.20 (s, 1H), 7.94 (d, J=8.4 Hz, 1H), 7.72 (s, 1H), 7.44-7.37 (m, 1H), 7.27 (d, J=6.8 Hz, 1H), 6.77 (s, 1H), 5.53-5.16 (m, 3H), 4.60-4.37 (m, 6H), 3.65-3.43 (m, 3H), 3.33 (s, 3H), 3.25-3.13 (m, 1H), 3.08 (s, 3H), 2.59-2.33 (m, 6H), 2.32-2.22 (m, 1H), 2.19-1.96 (m, 3H), 0.96 (t, J=7.4 Hz, 3H); LCMS (ESI, M+1): m/z=707.5.

### Example 771

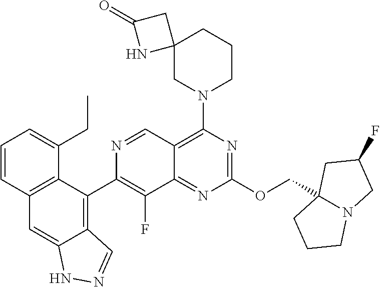

### 6-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-1,6-diazaspiro[3.5]nonan-2-one

The title compound was synthesized according to the 3-step procedure described for example 770 to produce the desired compound as a yellow solid (0.28 FA); ¹H NMR (400 MHz, METHANOL-d₄) δ=9.22 (s, 1H), 8.23 (s, 1H), 7.97 (d, J=8.4 Hz, 1H), 7.74 (s, 1H), 7.47-7.40 (m, 1H), 7.30 (br d, J=6.8 Hz, 1H), 5.51-5.35 (m, 1H), 4.55-4.32 (m, 4H), 4.05 (dd, J=8.8, 13.2 Hz, 1H), 3.98-3.79 (m, 1H), 3.65-3.45 (m, 3H), 3.25-3.16 (m, 1H), 3.00-2.88 (m, 1H), 2.79 (br d, J=14.8 Hz, 1H), 2.57-2.36 (m, 4H), 2.27-2.12 (m, 4H), 2.07-1.98 (m, 4H), 0.99 (br t, J=7.2 Hz, 3H); LCMS (ESI, M+1): m/z=639.4.

### Example 772

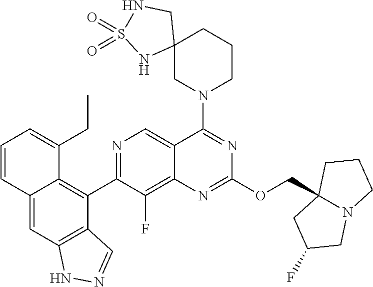 [SMILES: CCc1cccc2cc3[nH]ncc3c(-c3ncc4c(N5CCCC6(CNS(=O)(=O)N6)C5)nc(OC[C@@]56CCCN5C[C@H](F)C6)nc4c3F)c12]

### 7-(7-(5-ethyl-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,7-triazaspiro[4.5]decane 2,2-dioxide

The title compound was synthesized from Intermediate 22 according to the 2-step procedure described for example 767 to produce the desired compound as a yellow solid (0.60 formic acid salt); ¹H NMR (400 MHZ, METHANOL-d₄) δ=9.20 (d, J=8.0 Hz, 1H), 8.20 (d, J=3.6 Hz, 1H), 7.95 (br d, J=8.4 Hz, 1H), 7.85-7.68 (m, 1H), 7.45-7.38 (m, 1H), 7.28 (br d, J=6.4 Hz, 1H), 5.52-5.34 (m, 1H), 4.81-4.66 (m, 1H), 4.63-4.42 (m, 3H), 3.86-3.73 (m, 1H), 3.72-3.58 (m, 2H), 3.58-3.48 (m, 2H), 3.42-3.36 (m, 1H), 3.19 (br dd, J=12.0, 18.2 Hz, 2H), 2.57-2.41 (m, 3H), 2.40-2.24 (m, 2H), 2.22-2.05 (m, 4H), 2.02-1.80 (m, 3H), 0.99 (m, 3H); LCMS (ESI, M+1): m/z=690.1.

### Example 773

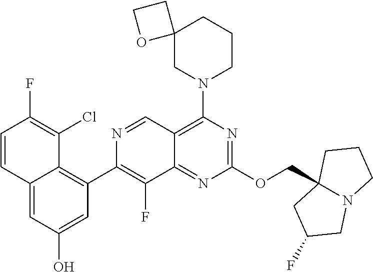 [SMILES: Oc1cc(-c2ncc3c(N4CCCC5(CCO5)C4)nc(OC[C@@]45CCCN4C[C@H](F)C5)nc3c2F)c2c(Cl)c(F)ccc2c1]

### 5-chloro-6-fluoro-4-(8-fluoro-2-(((2R,7aS)-2-fluorotetrahydro-1H-pyrrolizin-7a(5H)-yl)methoxy)-4-(1-oxa-6-azaspiro[3.5]nonan-6-yl)pyrido[4,3-d]pyrimidin-7-yl)naphthalen-2-ol

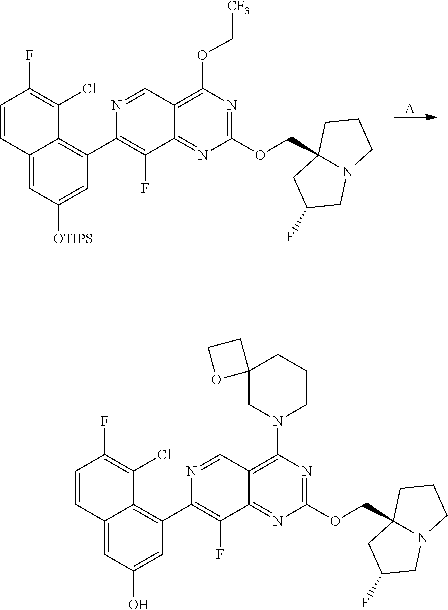

A mixture of 5-chloro-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (30 mg, 1.0 equiv), 1-oxa-8-azaspiro[3.5]nonane (11.6 mg, 2.0 equiv), DIPEA (17.5 mg, 4.0 equiv) and 4 Å molecular sieves (30 mg) in DMF (0.2 mL) was stirred at 40° C. for 12 hours under N₂ atmosphere. The mixture was filtered and the filtrate was concentrated and purified by prep-HPLC [C18, 0.1% formic acid condition] to afford the title compound (10.3 mg, 15.5% yield) as a yellow solid (0.40 formic acid salt). ¹H NMR (400 MHZ, CHLOROFORM-d) δ=9.28 (br d, J=11.0 Hz, 1H), 8.60-8.44 (m, 1H), 7.82 (dd, J=5.6, 9.2 Hz, 1H), 7.49-7.32 (m, 2H), 7.25 (s, 1H), 5.57-5.28 (m, 1H), 4.77-4.58 (m, 2H), 4.57-4.36 (m, 4H), 3.81 (br d, J=13.2 Hz, 1H), 3.73-3.38 (m, 4H), 3.22 (br s, 1H), 2.61-2.33 (m, 4H), 2.28 (br s, 2H), 2.15 (br d, J=2.2 Hz, 2H), 2.08-1.87 (m, 3H), 1.82 (br d, J=8.4 Hz, 1H); LCMS (ESI, M+1): m/z=626.4.

### Example 774

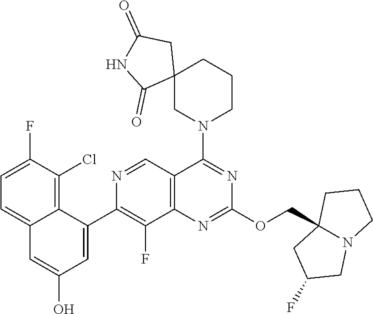 [SMILES: O=C1CC2(CCCN(c3nc(OC[C@@]45CCCN4C[C@H](F)C5)nc4c(F)c(-c5cc(O)cc6ccc(F)c(Cl)c56)ncc34)C2)C(=O)N1]

### 7-(7-(8-chloro-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-2,7-diazaspiro[4.5]decane-1,3-dione

The title compound was synthesized from Intermediate 24 according to the procedure described for example 773 to produce the desired compound as a white solid ¹H NMR (400 MHZ, methanol-d₄) δ=9.08-9.01 (m, 1H), 7.86-7.76 (m, 1H), 7.41-7.34 (m, 2H), 7.24-7.17 (m, 1H), 5.42-5.21 (m, 1H), 4.71-4.54 (m, 3H), 4.45-4.36 (m, 1H), 4.33-4.21 (m, 2H), 3.97-3.75 (m, 2H), 3.20-2.90 (m, 3H), 2.71-2.63 (m, 1H), 2.40-2.06 (m, 5H), 2.03-1.83 (m, 5H); LCMS (ESI, M+1): m/z=667.2.

### Example 775

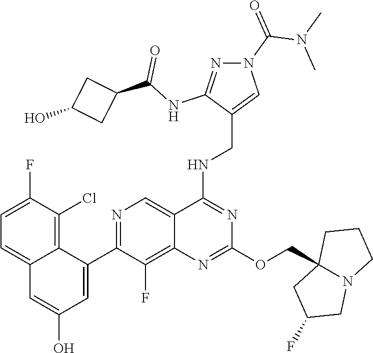 [SMILES: CN(C)C(=O)n1cc(CNc2nc(OC[C@@]34CCCN3C[C@H](F)C4)nc3c(F)c(-c4cc(O)cc5ccc(F)c(Cl)c45)ncc23)c(NC(=O)[C@H]2C[C@H](O)C2)n1]

### 4-(((7-(8-chloro-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-3-((1r,3S)-3-hydroxycyclobutanecarboxamido)-N,N-dimethyl-1H-pyrazole-1-carboxamide

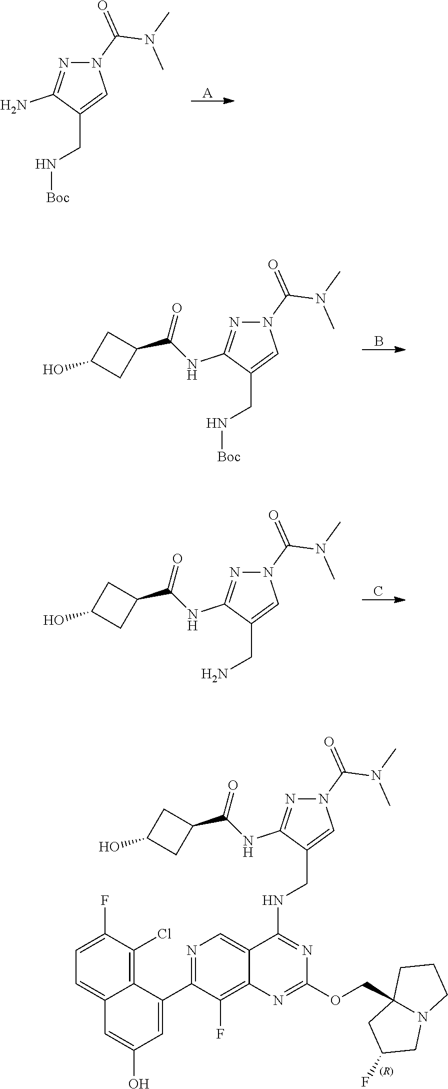

Step A. tert-butyl((1-(dimethylcarbamoyl)-3-((1r,3r)-3-hydroxycyclobutanecarboxamido)-1H-pyrazol-4-yl)methyl)carbamate: To a mixture of tert-butyl ((3-amino-1-(dimethylcarbamoyl)-1H-pyrazol-4-yl)methyl)carbamate (300 mg, 1.0 equiv) and (1r,3r)-3-hydroxycyclobutanecarboxylic acid (184 mg, 1.5 equiv) in DMF (9 mL) were added TEA (321 mg, 3.0 equiv) and T3P (1.01 g, 50% purity in EtOAc, 1.5 equiv). The reaction was stirred at 20° C. for 3 hours. The mixture was filtered and purified by reversed phase flash chromatography [C18, 0.1% formic acid condition] to afford the title compound (130 mg, 32% yield) as a yellow oil; ¹H NMR (400 MHZ, dimethylsulfoxide-d₆) δ=7.94-7.83 (m, 1H), 7.20-7.05 (m, 1H), 5.26-5.10 (m, 1H), 5.10-5.01 (m, 1H), 4.32-4.21 (m, 1H), 3.91-3.86 (m, 2H), 3.84 (br d, J=6.0 Hz, 1H), 3.11 (br s, 6H), 2.40-2.36 (m, 2H), 2.12-2.02 (m, 2H), 1.38-1.37 (m, 9H).

Step B. 4-(aminomethyl)-3-((1r,3r)-3-hydroxycyclobutanecarboxamido)-N,N-dimethyl-1H-pyrazole-1-carboxamide: To a mixture of tert-butyl((1-(dimethylcarbamoyl)-3-((1r,3r)-3-hydroxycyclobutanecarboxamido)-1H-pyrazol-4-yl)methyl)carbamate (130 mg, 1.0 equiv) in ACN (0.6 mL) was added HCl·dioxane (4 M, 1.28 mL, 15 equiv). The reaction was stirred at 20° C. for 0.5 hour. The mixture was concentrated to afford the title compound (108 mg, crude, HCl) as a yellow oil.

Step C. 4-(((7-(8-chloro-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)amino)methyl)-3-((1r,3S)-3-hydroxycyclobutanecarboxamido)-N,N-dimethyl-1H-pyrazole-1-carboxamide: To a mixture of 7-(8-chloro-7-fluoro-3-((triisopropylsilyl)oxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (64 mg, 1.0 equiv) and 4-(aminomethyl)-3-((1r,3r)-3-hydroxycyclobutanecarboxamido)-N,N-dimethyl-1H-pyrazole-1-carboxamide (108 mg, 4.0 equiv, HCl) in ACN (0.5 mL) and DMF (0.5 mL) were added K₃PO₄ (144 mg, 8.0 equiv) and 4 Å molecular sieves (40 mg). The reaction was stirred at 40° C. for 12 hours. The mixture was concentrated and purified by prep-HPLC [Phenomenex luna C 18 150×25 mm×10 μm; A: water (FA), B: ACN; B %: 15%-45% over 10 min] to afford the title compound (30 mg, 43% yield) as a white solid; SFC: >99% ee, Column: Chiralcel OJ-3 50×4.6 mm I.D., 3 μm; Gradient elution: MeOH (0.05% DEA) in CO₂ from 5% to 40%; 3 mL/min; 220 nm; t_{R}: 1.579 min; ¹H NMR (400 MHz, dimethylsulfoxide-d₆ δ=10.29-10.20 (m, 1H), 10.09-9.99 (m, 1H), 9.31-9.26 (m, 1H), 9.25-9.18 (m, 1H), 8.18-8.15 (m, 1H), 7.97-7.90 (m, 1H), 7.59-7.52 (m, 1H), 7.42 (d, J=1.6 Hz, 1H), 7.20-7.15 (m, 1H), 5.42-5.23 (m, 1H), 4.62-4.55 (m, 2H), 4.28-4.19 (m, 2H), 4.18-4.09 (m, 1H), 3.12 (br s, 6H), 2.95-2.87 (m, 1H), 2.38-2.28 (m, 4H), 2.24-2.11 (m, 2H), 2.10-1.97 (m, 4H), 1.96-1.75 (m, 4H); ¹H NMR (400 MHZ, dimethylsulfoxide-d₆+deuterium oxide-d₂) δ=9.26-9.21 (m, 1H), 8.19-8.11 (m, 1H), 7.94-7.87 (m, 1H), 7.58-7.50 (m, 1H), 7.45-7.40 (m, 1H), 7.19-7.15 (m, 1H), 5.42-5.24 (m, 1H), 4.63-4.53 (m, 2H), 4.15 (s, 3H), 3.31-3.20 (m, 2H), 3.20-3.15 (m, 1H), 3.09 (br s, 6H), 2.97-2.89 (m, 1H), 2.36-2.27 (m, 3H), 2.25-2.19 (m, 1H), 2.16-2.12 (m, 1H), 2.09-1.97 (m, 3H), 1.96-1.75 (m, 3H); 19F NMR (376 MHz, dimethylsulfoxide-d₆+deuterium oxide-d₂) δ=−114.879, −140.224, −172.016; LCMS (ESI, M+1): m/z=780.1.

### Example 776

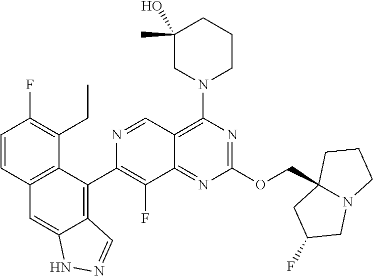 [SMILES: CCc1c(F)ccc2cc3[nH]ncc3c(-c3ncc4c(N5CCC[C@@](C)(O)C5)nc(OC[C@@]56CCCN5C[C@H](F)C6)nc4c3F)c12]

### (3R)-1-(7-(5-ethyl-6-fluoro-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol

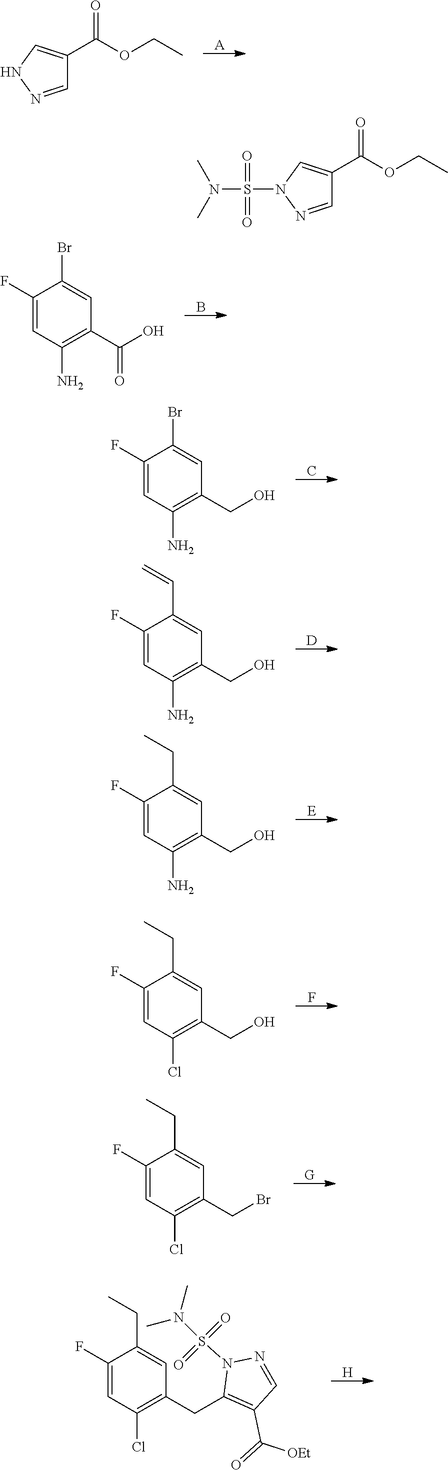

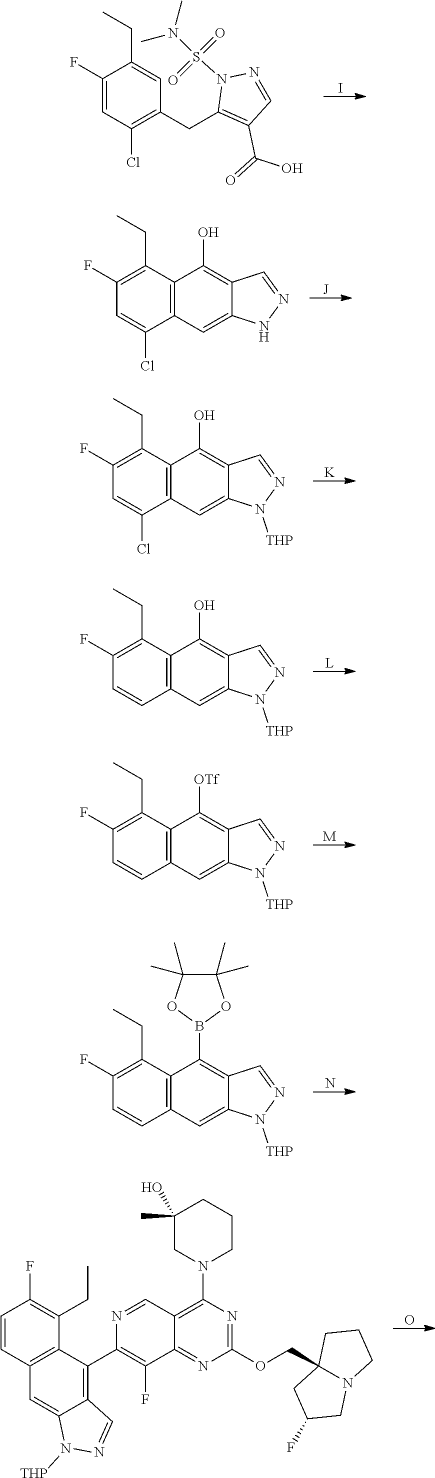

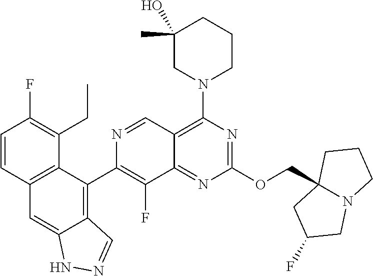 [SMILES: CCc1c(F)ccc2cc3[nH]ncc3c(-c3ncc4c(N5CCC[C@@](C)(O)C5)nc(OC[C@@]56CCCN5C[C@H](F)C6)nc4c3F)c12]

Step A: ethyl 1-(N,N-dimethylsulfamoyl)-1H-pyrazole-4-carboxylate: To a solution of ethyl 1H-pyrazole-4-carboxylate (150 g, 1.0 equiv) and DABCO (132 g, 1.1 equiv) in MeCN (1500 mL) was added N,N-dimethylsulfamoyl chloride (169 g, 1.1 eq) at 20° C. portionwise. The reaction was stirred at 20° C. for 2 hours. The mixture was concentrated under vacuum at 40° C. The mixture was diluted with water (800 mL) and extracted with EtOAc (3×500 mL). The combined organic layers were washed with brine (500 mL), dried over anhydrous sodium sulfate, concentrated, and purified with column chromatography (SiO₂, petroleum ether/ethyl acetate 10:1-3:1) to afford the title compound (550 g, 99% yield) as a white solid; LCMS (ESI, M+1): m/z=248.0.

Step B: (2-amino-5-bromo-4-fluorophenyl)methanol: To a solution of 2-amino-5-bromo-4-fluorobenzoic acid (90.0 g, 1.0 equiv) in THF (1000 mL) was added BH₃·Me₂S (115 mL, 10 M, 3.0 equiv) at 0° C. The reaction was stirred at 20° C. for 12 hours. The mixture was quenched by slow addition of MeOH (700 mL) at 0° C. The mixture was concentrated and diluted with EtOAc (300 mL). The combined organic layers were washed with brine (300 mL), dried over anhydrous sodium sulfate, concentrated, and purified by column chromatography (SiO₂, petroleum ether/ethyl acetate 10:1-3:1) to afford the title compound (92.0 g, crude) as a yellow solid.

Step C. (2-amino-4-fluoro-5-vinylphenyl)methanol: To a mixture of (2-amino-5-bromo-4-fluorophenyl)methanol (170 g, 1.0 equiv), 4,4,5,5-tetramethyl-2-vinyl-1,3,2-dioxaborolane (259 g, 2.5 equiv) and Cs₂CO₃ (755 g, 3.0 equiv) in dioxane (1500 mL), H₂O (500 mL) was added bis-triphenylphosphine-palladium(II) chloride (28.3 g, 0.050 equiv). The reaction was degassed and stirred at 90° C. for 12 hours under nitrogen atmosphere. The mixture was diluted with H₂O (900 mL) and extracted with EtOAc (2×1000 mL). The combined organic layers were washed with brine (400 mL), dried over anhydrous sodium sulfate, concentrated, and purified by column chromatography (SiO₂, petroleum ether/ethyl acetate 1:3) to afford the title compound (124 g, 96% yield) as a yellow solid; LCMS (ESI, M−17): m/z=150.0.

Step D. (2-amino-5-ethyl-4-fluorophenyl)methanol: To a mixture of (2-amino-4-fluoro-5-vinylphenyl)methanol (93.0 g, 1.0 equiv) in MeOH (900 mL) was added Pd/C (4.5 g, 5% purity) under nitrogen atmosphere. The suspension was degassed and purged with H₂ for 3 times. The reaction was stirred under H₂ (15 Psi) at 20° C. for 24 hours. The mixture was filtered and purified by reversed phase flash chromatography [C18, 0.1% formic acid condition] to afford the title compound (52.8 g, 49% yield) as a yellow solid; LCMS (ESI, M−17): m/z=152.0.

Step E. (2-chloro-5-ethyl-4-fluorophenyl)methanol: To a cooled solution of (2-amino-5-ethyl-4-fluorophenyl)methanol (75.0 g, 1.0 equiv) in MeCN (1000 mL), H₂O (500 mL) and HCl (258 mL, 12 M, 7.0 equiv) was added dropwise a solution of NaNO₂ (36.7 g, 1.2 equiv) in H₂O (250 mL). The reaction was stirred at 0° C. for 1 hour. CuCl (263 g, 6.0 equiv) was added to the cooled mixture. The reaction was stirred at 70° C. for another 1 hour. The mixture was diluted with H₂O (100 mL). The mixture was concentrated under reduced pressure to remove MeCN. The mixture was filtered to remove residue. The mixture was diluted with H₂O (50 mL) and extracted with EtOAc (2×1000 mL). The combined organic layers were washed with brine (100 mL), dried over anhydrous sodium sulfate, concentrated, and purified by column chromatography (SiO₂, petroleum ether/ethyl acetate 5:1) to afford the title compound (28.9 g, 32% yield) as yellow solid; LCMS (ESI, M−17): m/z=171.0.

Step F. 1-(bromomethyl)-2-chloro-5-ethyl-4-fluorobenzene: A mixture of (2-chloro-5-ethyl-4-fluorophenyl)methanol (15.0 g, 1.0 equiv) in DCM (100 mL) and PBr₃ (43.1 g, 2.0 equiv) was stirred at 0° C. for 1 hour. The mixture was quenched with water (2×50 mL) and extracted with EtOAc (2×100 mL). The combined organic layers were washed with brine (50 mL), dried over anhydrous sodium sulfate, concentrated, and purified by column chromatography (SiO₂, petroleum ether/ethyl acetate 50:1 to 10:1) to afford the title compound (10.8 g, 51% yield) as a white oil; ¹H NMR (400 MHZ, METHANOL-d₄) δ=7.41 (d, J=8.0 Hz, 1H), 7.15 (d, J=9.6 Hz, 1H), 4.60 (s, 2H), 2.69-2.58 (m, 2H), 1.23-1.18 (m, 3H).

Step G. ethyl 5-(2-chloro-5-ethyl-4-fluorobenzyl)-1-(N,N-dimethylsulfamoyl)-1H-pyrazole-4-carboxylate: To a solution of 1-(bromomethyl)-2-chloro-5-ethyl-4-fluorobenzene (6.89 g, 1.0 equiv) in THF (50 mL) were added LDA (18.1 mL, 2 M, 1.3 equiv) and HMPA (6.49 g, 1.3 equiv) at −78° C. for 0.5 hour. Then 4-(1-ethoxyvinyl)-N,N-dimethyl-1H-pyrazole-1-sulfonamide (5.61 g, 0.80 equiv) was added into the mixture. The reaction was stirred at −78° C. for 0.5 hour. The reaction was warmed to 20° C. and stirred at 20° C. for 0.5 hour. The mixture was quenched with water (50 mL) at 0° C. and extracted with ethyl acetate (100 mL). The organic layer was washed with brine (50 mL), dried over anhydrous sodium sulfate, concentrated, and purified with prep-HPLC (column: YMC Triart C18 250×50 mm×7 μm; mobile phase: [water (FA)-ACN]; B %: 55%-75% over 20 min) to afford the title compound (5.28 g, 43% yield) as a yellow solid; LCMS (ESI, M+1): m/z=418.0.

Step H. 5-(2-chloro-5-ethyl-4-fluorobenzyl)-1-(N,N-dimethylsulfamoyl)-1H-pyrazole-4-carboxylic acid: To a mixture of ethyl 5-(2-chloro-5-ethyl-4-fluorobenzyl)-1-(N,N-dimethylsulfamoyl)-1H-pyrazole-4-carboxylate (5.70 g, 1.0 equiv) in dioxane (50 mL) and H₂O (50 mL) was added NaOH (10.9 g, 20 equiv). The reaction was stirred at 100° C. for 2 hours. The mixture was diluted with ethyl acetate (3×50 mL) and separated. The organic layer was dried over anhydrous sodium sulfate and concentrated to afford the title compound (7.5 g, crude) as a yellow solid and used for the next step without further purification; LCMS (ESI, M+1): m/z=389.9.

Step I. 8-chloro-5-ethyl-6-fluoro-1H-benzo[f]indazol-4-ol: The mixture of 5-(2-chloro-5-ethyl-4-fluorobenzyl)-1-(N,N-dimethylsulfamoyl)-1H-pyrazole-4-carboxylic acid (4.40 g, 1.0 equiv) in CF₃SO₃H (44 mL) was stirred at 90° C. for 2 hours. The mixture was diluted with NaHCO₃ aqueous solution and extracted with ethyl acetate (3×30 mL). The organic layer was dried over anhydrous sodium sulfate, concentrated, and purified with prep-HPLC (column: Phenomenex Luna C18 100×30 mm×5 μm; mobile phase: [water (FA)-ACN]; B %: 19%-49% over 8 min) to afford the title compound (1.17 g, 39% yield) as a yellow solid; LCMS (ESI, M+1): m/z=264.8.

Step J: 8-chloro-5-ethyl-6-fluoro-1-(tetrahydro-2H-pyran-2-yl)-1H-benzo[f]indazol-4-ol: To a solution of 8-chloro-5-ethyl-6-fluoro-1H-benzo[f]indazol-4-ol (1.80 g, 1.0 equiv) and DHP (1.14 g, 2.0 equiv) in THF (20 mL) was added TsOH·H₂O (12.9 mg, 0.010 equiv). The reaction was stirred at 20° C. for 2 hours. The mixture was diluted with water (3×5 mL) and extracted with EtOAc (3×15 mL). The combined organic layers were washed with brine (15 mL), dried over anhydrous sodium sulfate, concentrated, and purified by reversed phase flash chromatography (C18, 0.1% formic acid condition) to afford the title compound (1.0 g, 42% yield) as a yellow solid. LCMS (ESI, M−83): m/z=265.0.

Step K: 5-ethyl-6-fluoro-1-(tetrahydro-2H-pyran-2-yl)-1H-benzo[f]indazol-4-ol: To a solution of 8-chloro-5-ethyl-6-fluoro-1-(tetrahydro-2H-pyran-2-yl)-1H-benzo[f]indazol-4-ol (2.90 g, 1.0 equiv) and NaHCO₃ (2.10 g, 3.0 equiv) in MeOH (50 mL) was added Pd/C (1.0 g, 10% purity) under N₂ atmosphere. The suspension was degassed and purged with H₂ for 3 times. The reaction was stirred under H₂ (15 Psi) at 20° C. for 12 hours. The mixture was diluted with H₂O (40 mL). The mixture was concentrated at 25° C. The mixture was extracted with dichloromethane (3×40 mL) and dried over anhydrous sodium sulfate, concentrated at 25° C. under reduced pressure and purified with reversed phase flash chromatography (C18, 0.1% formic acid condition) to afford the title compound (2.2 g, 84% yield) as a yellow solid. LCMS (ESI, M−83): m/z=231.0.

Step L: 5-ethyl-6-fluoro-1-(tetrahydro-2H-pyran-2-yl)-1H-benzo[f]indazol-4-yl trifluoromethanesulfonate: To a solution of 5-ethyl-6-fluoro-1-(tetrahydro-2H-pyran-2-yl)-1H-benzo[f]indazol-4-ol (1.10 g, 1.0 equiv), DIPEA (1.81 g, 4.0 equiv) and 4 Å molecular sieves (50.0 mg) in DCM (20 mL) was added Tf₂O (1.97 g, 2.0 equiv) at −40° C. The reaction was stirred at −40° C. for 0.5 hour. The mixture was diluted with water (3×30 mL) and extracted with EtOAc dichloromethane (3× 30 mL). The combined organic layers were dried over anhydrous sodium sulfate, concentrated at 25° C. under reduced pressure, and purified with column (Al₂O₃, petroleum ether/ethyl acetate 20:1-5:1) to afford the title compound (1.0 g, crude) as a yellow solid. LCMS (ESI, M−83): m/z=362.7.

Step M: 5-ethyl-6-fluoro-1-(tetrahydro-2H-pyran-2-yl)-4-(4,4,5,5-tetramethyl-1,3,2-dioxaborolan-2-yl)-1H-benzo[f]indazole: To a solution of 5-ethyl-6-fluoro-1-(tetrahydro-2H-pyran-2-yl)-1H-benzo[f]indazol-4-yl trifluoromethanesulfonate (800 mg, 1.0 equiv), 4,4,5,5-tetramethyl-1,3,2-dioxaborolane (2.29 g, 10 equiv) and TEA (544 mg, 3.0 equiv) in MeCN (10 mL) was added bis(triphenylphosphine)palladium(II) chloride (131 mg, 0.10 equiv). The reaction was stirred at 80° C. for 3 hours. The mixture was diluted with water (3×15 mL) and extracted with dichloromethane (3×15 mL). The combined organic layers were dried over anhydrous sodium sulfate, concentrated, and purified with reversed phase flash chromatography (C18, 0.1% formic acid condition) to afford the title compound (120 mg, 16% yield) as a yellow solid. LCMS (ESI, M+1): m/z=425.2.

Step N: (3R)-1-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol: To a solution of (R)-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (100 mg, 1.0 equiv), 5-ethyl-6-fluoro-1-(tetrahydro-2H-pyran-2-yl)-4-(4,4,5,5-tetramethyl-1,3,2-dioxaborolan-2-yl)-1H-benzo[f]indazole (112 mg, 1.2 equiv) and K₃PO₄ (1.5 M, 3.0 equiv) in methoxycyclopentane (2 mL) was added CataCXium A Pd G3 (16.1 mg, 0.10 equiv). The reaction was stirred at 90° C. for 4 hours. The mixture was diluted with water (3×5 mL) and extracted with dichloromethane (3×5 mL). The combined organic layers were dried over anhydrous sodium sulfate, concentrated, and purified with reversed phase flash chromatography (C18, 0.1% formic acid condition) to afford the title compound (68 mg, 42% yield) as a yellow oil. LCMS (ESI, M+1): m/z=716.5.

Step O: (3R)-1-(7-(5-ethyl-6-fluoro-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol: To a solution of (3R)-1-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol (35.0 mg, 1.0 equiv) in DCM (0.6 mL) was added TFA (924 mg, 166 equiv). The mixture was stirred at 20° C. for 10 minutes. The mixture was concentrated at 20° C. The residue was adjusted with saturated NaHCO₃ aqueous solution to pH˜7, extracted with dichloromethane (2×3 mL). The combined organic layer was concentrated and purified with prep-HPLC (column: Welch Xtimate C18 150×25 mm×5 μm; mobile phase: [water (TFA)-ACN]; B %: 18%-48% over 10 min) to afford the title compound (9.78 mg, 31% yield, TFA) as an off-white solid; ¹H NMR (400 MHZ, METHANOL-d₄) δ=9.41 (d, J=3.6 Hz, 1H), 8.26 (s, 1H), 8.02 (dd, J=6.0, 9.2 Hz, 1H), 7.71 (d, J=16.4 Hz, 1H), 7.34 (t, J=9.2 Hz, 1H), 5.66-5.48 (m, 1H), 4.78-4.70 (m, 2H), 4.69 (s, 1H), 4.42 (br t, J=14.8 Hz, 1H), 4.11-3.95 (m, 1H), 3.94-3.80 (m, 2H), 3.74-3.57 (m, 1H), 3.54-3.40 (m, 2H), 2.80-2.56 (m, 3H), 2.49-2.28 (m, 4H), 2.25-2.09 (m, 2H), 1.95-1.76 (m, 3H), 1.32 (d, J=6.8 Hz, 3H), 0.92-0.81 (m, 3H); LCMS (ESI, M+1): m/z=632.3.

### Example 777

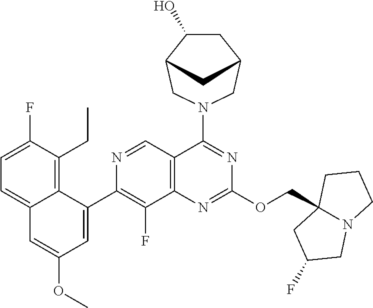 [SMILES: CCc1c(F)ccc2cc(OC)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(8-ethyl-7-fluoro-3-methoxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

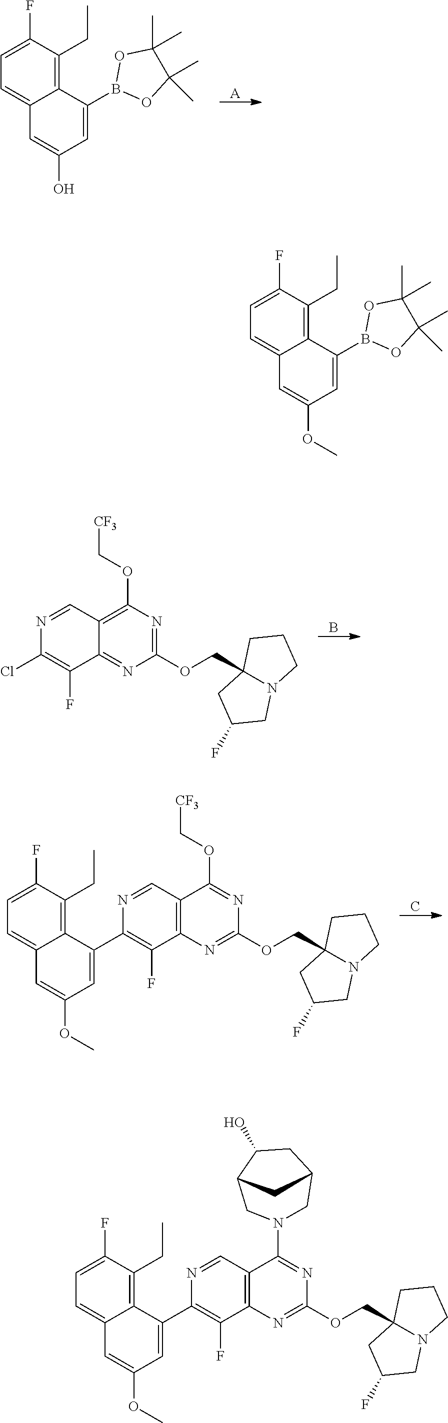

Step A. 2-(8-ethyl-7-fluoro-3-methoxynaphthalen-1-yl)-4,4,5,5-tetramethyl-1,3,2-dioxaborolane: To a solution of 5-ethyl-6-fluoro-4-(4,4,5,5-tetramethyl-1,3,2-dioxaborolan-2-yl)naphthalen-2-ol (5.0 g, 1.0 equiv) in MeOH (50 mL) was added slowly diazomethyl(trimethyl) silane (2 M, 23.7 mL, 3.0 equiv) during period of 30 minutes. The mixture was stirred at 15° C. for 24 hours. The mixture was diluted with water (20 mL) and extracted with ethyl acetate (4× 20 mL). The organic phase was dried over anhydrous sodium sulfate, concentrated and purified by column chromatography [SiO₂, Petroleum ether/Ethyl acetate 100:1 to 50:1] to afford the title compound (4.50 g, 85% yield) as a white solid; ¹H NMR (400 MHZ, METHANOL-d₄) δ=7.72-7.54 (m, 1H), 7.40-6.99 (m, 3H), 3.88 (s, 3H), 3.11-3.01 (m, 2H), 1.42 (br s, 12H), 1.25-1.26 (m, 3H); LCMS (ESI, M+1): m/z=331.3

Step B. 7-(8-ethyl-7-fluoro-3-methoxynaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: A mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (4.50 g, 1.0 equiv), Cs₂CO₃ (1.5 M, 20.5 mL, 3.0 equiv), 2-(8-ethyl-7-fluoro-3-methoxynaphthalen-1-yl)-4,4,5,5-tetramethyl-1,3,2-dioxaborolane (4.06 g, 1.2 equiv) in methoxycyclopentane (100 mL) was degassed and then AdanBuP-Pd-G3 (747 mg, 0.1 equiv) was added. The mixture was stirred at 100° C. for 12 hours under N₂ atmosphere. The mixture was diluted with water (10 mL) and extracted with ethyl acetate (4×10 mL). The organic phase was dried over anhydrous sodium sulfate, concentrated and purified by reversed phase flash chromatography [C18, 0.1% formic acid condition] to afford the title compound (3.0 g, 46% yield) as a yellow oil; LCMS (ESI, M+1): m/z=607.1

Step C. (1R,5R,6R)-3-(7-(8-ethyl-7-fluoro-3-methoxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 7-(8-ethyl-7-fluoro-3-methoxynaphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (1.50 g, 1.0 equiv) and (1R,5R,6R)-3-azabicyclo[3.2.1]octan-6-ol (472 mg, 1.5 equiv) in DMF (5 mL) was added DIPEA (959 mg, 3.0 equiv). The mixture was stirred at 40° C. for 12 hours. The reaction mixture was filtered and purified with prep-HPLC [column: Kromasil Eternity XT 250×80 mm×10 μm; A: water (10 mM NH₄HCO₃), B: ACN, B %: 50%-80% over 20 min] and lyophilized to afford the title compound (1.50 g, 95% yield) as yellow solid; ¹H NMR (400 MHZ, METHANOL-d₄) δ=9.23 (d, J=19.2 Hz, 1H), 7.83-7.79 (m, 1H), 7.45 (d, J=2.8 Hz, 1H), 7.32-7.27 (m, 1H), 7.14-7.10 (m, 1H), 5.37-5.23 (m, 1H), 5.03-4.97 (m, 1H), 4.78-4.75 (m, 1H), 4.35-4.29 (m, 2H), 4.23-4.19 (m, 1H), 3.95 (s, 3H), 3.83-3.76 (m, 1H), 3.50-3.43 (m, 1H), 3.28-3.17 (m, 3H), 3.03-3.00 (m, 1H), 2.55-2.47 (m, 1H), 2.40-2.30 (m, 2H), 2.26-2.10 (m, 5H), 2.02-1.86 (m, 4H), 1.82-1.80 (m, 1H), 1.42-1.38 (m, 1H), 0.83-0.77 (m, 3H); LCMS (ESI, M+1): m/z=634.2.

### Example 778

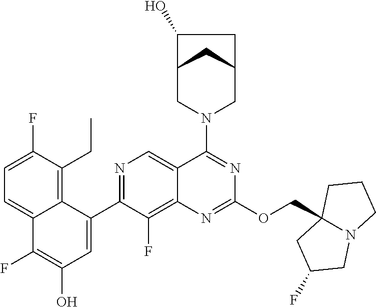 [SMILES: CCc1c(F)ccc2c(F)c(O)cc(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(8-ethyl-4,7-difluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

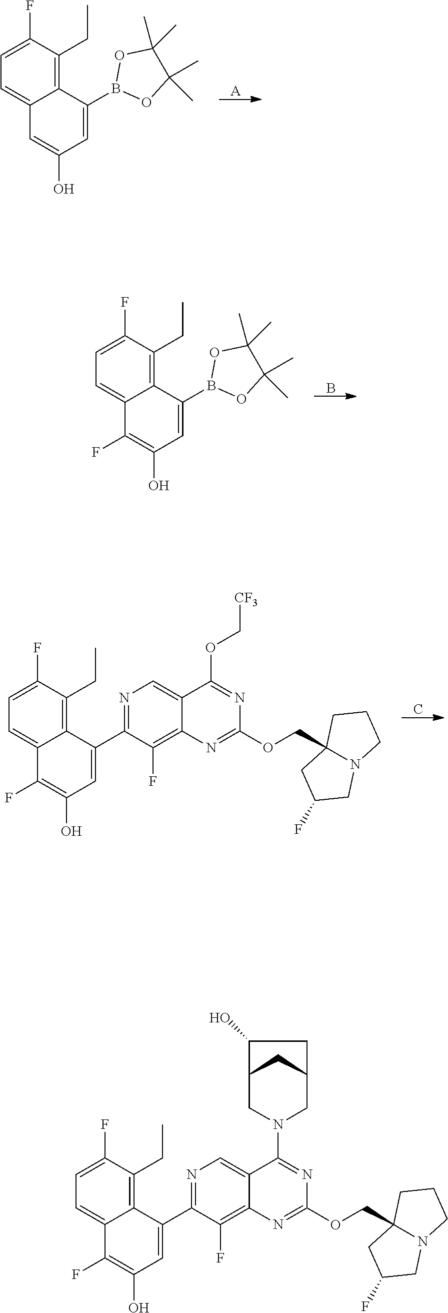

Step A. 5-ethyl-1,6-difluoro-4-(4,4,5,5-tetramethyl-1,3,2-dioxaborolan-2-yl)naphthalen-2-ol: To a solution of 5-ethyl-6-fluoro-4-(4,4,5,5-tetramethyl-1,3,2-dioxaborolan-2-yl)naphthalen-2-ol (1.50 g, 1.0 equiv) in MeCN (15 mL) was added Select F (1.68 g, 1.0 equiv) at 0° C. The reaction was stirred at 0-20° C. for 12 hours. The mixture was filtered and purified with reversed-phase HPLC (0.1% FA condition) to afford the title compound (650 mg, 40% yield) as a brown solid; ¹H NMR (400 MHZ, DMSO-d₆) δ=10.06 (s, 1H), 7.85 (dd, J=6.0, 9.2 Hz, 1H), 7.45-7.35 (m, 2H), 3.03 (dq, J=2.0, 7.6 Hz, 2H), 1.42-1.30 (m, 12H), 1.21-1.15 (m, 3H); LCMS (ESI, M+1): m/z=334.9.

Step B. 5-ethyl-1,6-difluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol: A mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (300 mg, 1.0 equiv), 5-ethyl-1,6-difluoro-4-(4,4,5,5-tetramethyl-1,3,2-dioxaborolan-2-yl)naphthalen-2-ol (343 mg, 1.5 equiv), CataCXium A Pd G3 (49.8 mg, 0.10 equiv) and Cs₂CO₃ (668 mg, 3.0 equiv) in methoxycyclopentane (3 mL) and H₂O (0.75 mL) was degassed and stirred at 90° C. for 2 hours under nitrogen atmosphere. The mixture was diluted with H₂O (3 mL) and extracted with EtOAc (3×5 mL). The combined organic layers were washed with brine (2×5 mL), dried over anhydrous sodium sulfate, concentrated, and purified with reversed-phase HPLC (0.1% FA condition) to afford the title compound (80.0 mg, 12% yield) as a yellow solid; LCMS (ESI, M+1): m/z=611.3.

Step C. (1R,5R,6R)-3-(7-(8-ethyl-4,7-difluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of 5-ethyl-1,6-difluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (80.0 mg, 1.0 equiv) and (1R,5R,6R)-3-azabicyclo[3.2.1]octan-6-ol (25.0 mg, 1.5 equiv) in DMF (0.5 mL) were added DIPEA (371 mg, 22 equiv) and 4 Å molecular sieves (10.0 mg). The reaction was stirred at 40° C. for 12 hours. The mixture was filtered and purified by reversed-phase HPLC (0.1% FA condition) and purified by prep-HPLC (column: Welch Xtimate C18 150×25 mm×5 μm; mobile phase: [water (NH₃·H₂O)-ACN]; B %: 22%-52%, 8 min) to afford the title compound (23.8 mg, 28% yield) as a yellow solid; ¹H NMR (400 MHz, METHANOL-d₄) δ=9.22 (d, J=19.6 Hz, 1H), 7.99 (dd, J=6.0, 9.2 Hz, 1H), 7.35 (t, J=9.2 Hz, 1H), 7.18 (dd, J=8.8, 14.4 Hz, 1H), 5.39-5.22 (m, 1H), 4.68-4.53 (m, 2H), 4.37-4.28 (m, 2H), 4.25-4.17 (m, 1H), 3.85-3.74 (m, 1H), 3.46 (br dd, J=13.2, 16.8 Hz, 1H), 3.30-3.16 (m, 3H), 3.06-2.95 (m, 1H), 2.55-2.11 (m, 8H), 2.04-1.75 (m, 5H), 1.40 (br d, J=13.6 Hz, 1H), 0.91-0.69 (m, 3H); LCMS (ESI, M+1): m/z=638.4.

### Example 779

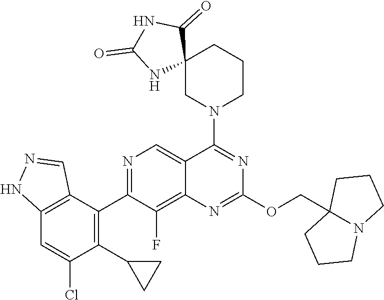 [SMILES: O=C1NC(=O)[C@]2(CCCN(c3nc(OCC45CCCN4CCC5)nc4c(F)c(-c5c(C6CC6)c(Cl)cc6[nH]ncc56)ncc34)C2)N1]

### (5R)-7-(7-(6-chloro-5-cyclopropyl-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione

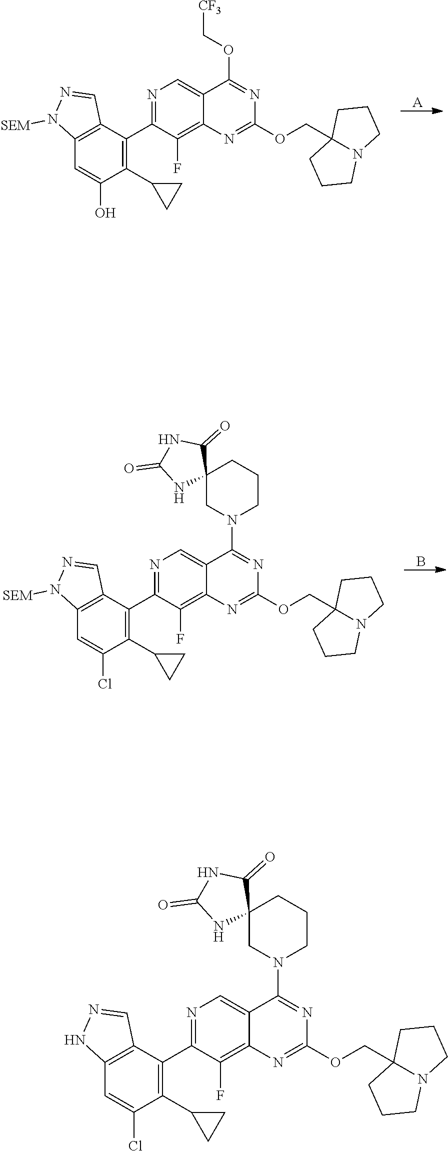

Step A. (5R)-7-(7-(6-chloro-5-cyclopropyl-1-((2-(trimethylsilyl) ethoxy)methyl)-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a solution of 7-(6-chloro-5-cyclopropyl-1-((2-(trimethylsilyl) ethoxy)methyl)-1H-indazol-4-yl)-8-fluoro-2-((hexahydro-1H-pyrrolizin-7a-yl)methoxy)-4-(2,2,2-trifluoroethoxy)pyrido[4,3-d]pyrimidine (60.0 mg, 1.0 equiv) and (5R)-1,3,9-triazaspiro[4.5]decane-2,4-dione (43.1 mg, 3.0 equiv) in DMF (0.5 mL) was added DIPEA (32.9 mg, 3.0 equiv). The mixture was stirred at 40° C. for 12 hours. The mixture was filtered, concentrated and purified by reversed phase flash chromatography [C18, 0.1% formic acid condition] followed by prep-HPLC [Phenomenex C18 75×30 mm×3 μm; A: water (10 mM FA), B: ACN, 2%-32% over 7 min] and lyophilized to afford the title compound (50.0 mg, 76% yield) as an off-white solid; LCMS (ESI, M+1): m/z=776.3.

Step B. (5R)-7-(7-(6-chloro-5-cyclopropyl-1H-indazol-4-yl)-8-fluoro-2-((hexahydro-1H-pyrrolizin-7a-yl)methoxy)pyrido[4.3-d]pyrimidin-4-yl)-1,3,7-triazaspiro[4.5]decane-2,4-dione: To a solution of (5R)-7-(7-(6-chloro-5-cyclopropyl-1-((2-(trimethylsilyl) ethoxy)methyl)-1H-indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione (50.0 mg, 1.0 equiv) in DCM (0.25 mL) was added TFA (1.93 g, 1.25 mL, 262 equiv). The mixture was stirred at 20° C. for 10 minutes. The reaction mixture was concentrated and purified with prep-HPLC [Unisil 3-100 C18 Ultra 150×50 mm×3 μm; A: water (10 mM FA), B: ACN, B %: 12%-42% over 7 min] and lyophilized to afford the title compound (11.4 mg, 26% yield) as an off-white solid (0.61 formic acid salt); ¹H NMR (400 MHZ, METHANOL-d₄) δ=9.26 (d, J=1.6 Hz, 1H), 7.81 (d, J=6.8 Hz, 2H), 4.72-4.63 (m, 1H), 4.57-4.46 (m, 3H), 3.95-3.76 (m, 2H), 3.51-3.42 (m, 2H), 3.13-2.99 (m, 2H), 2.33-2.20 (m, 3H), 2.17-1.93 (m, 10H), 0.97-0.86 (m, 1H), 0.70-0.53 (m, 1H), 0.35-0.24 (m, 1H), 0.17-0.02 (m, 1H); LCMS (ESI, M+1): m/z=646.2.

### Example 780

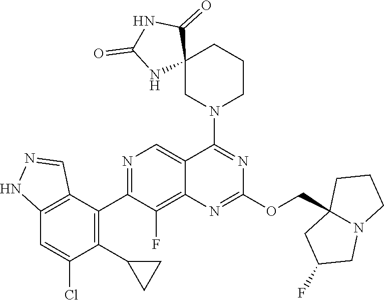 [SMILES: O=C1NC(=O)[C@]2(CCCN(c3nc(OC[C@@]45CCCN4C[C@H](F)C5)nc4c(F)c(-c5c(C6CC6)c(Cl)cc6[nH]ncc56)ncc34)C2)N1]

### (5R)-7-(7-(6-chloro-5-cyclopropyl-1H-indazol-4-yl)-8-fluoro-2-(((2R,7aS)-2-fluorohexahydro-1H-pyrrolizin-7a-yl)methoxy)pyrido[4,3-d]pyrimidin-4-yl)-1,3,7-triazaspiro[4.5]decane-2,4-dione

The title compound was synthesized from Intermediate 25 according to the 2-step procedure described for example 779 to produce the desired compound as yellow solid (0.38FA); ¹H NMR (400 MHz, methanol-d4) δ=9.24 (d, J=2.4 Hz, 1H), 7.80 (d, J=9.6 Hz, 2H), 5.47 (d, J=36.0 Hz, 1H), 4.69-4.62 (m, 1H), 4.49-4.36 (m, 3H), 3.85-3.72 (m, 2H), 3.48-3.35 (m, 4H), 3.25-3.11 (m, 1H), 2.53-2.33 (m, 2H), 2.30-2.20 (m, 2H), 2.17-2.07 (m, 3H), 2.05-1.92 (m, 3H), 1.03-0.80 (m, 1H), 0.69-0.50 (m, 1H), 0.34-0.19 (m, 1H), 0.10-0.06 (m, 1H); LCMS (ESI, M+1, M+3): m/z=664.2.

### Example 781

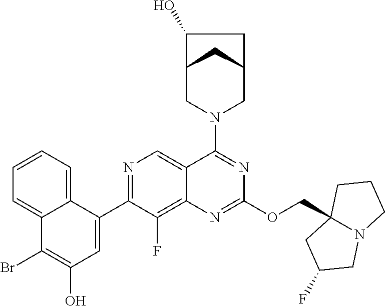 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1Br]

### (1R,5R,6R)-3-(7-(4-bromo-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol

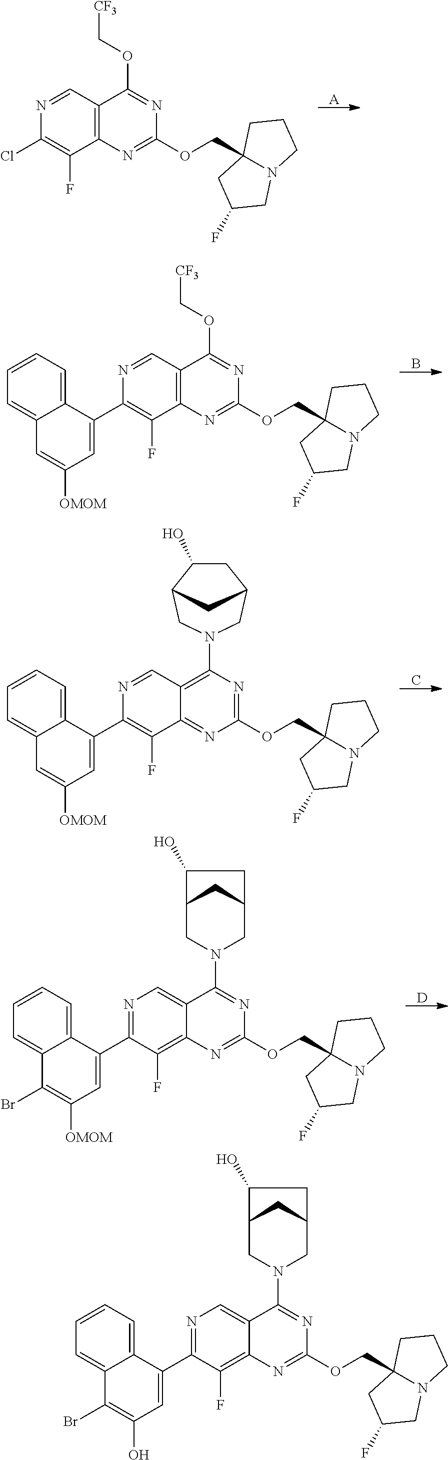

Step A. 8-fluoro-2-(((2R,7aS)-2-fluorohexahydro-1H-pyrrolizin-7a-yl)methoxy)-7-(3-(methoxymethoxy)naphthalen-1-yl)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (1.00 g, 2.28 mmol, 1.0 equiv) and 2-[3-(methoxymethoxy)-1-naphthyl]-4,4,5,5-tetramethyl-1,3,2-dioxaborolane (859 mg, 2.73 mmol, 1.2 equiv) in methoxycyclopentane (50 mL) under N₂ were added CataCxium A Pd G3 (166 mg, 228 μmol, 0.1 equiv) and Cs₂CO₃ (1.5 M, 4.56 mL, 3.0 equiv). The reaction was stirred at 100° C. for 6 hours under N₂. The reaction mixture was diluted with H₂O (25 mL) and extracted with EtOAc (25 mL×3). The combined organic layers were washed with brine (20 mL×1), dried over Na₂SO₄, filtered, concentrated and purified by reversed-phase HPLC [0.1% NH₃·H₂O] to afford the title compound (640 mg, 1.02 mmol, 44% yield, 94% purity) as a yellow solid; LCMS (ESI, M+1): m/z=591.4.

Step B. (1R,5R,6R)-3-(8-fluoro-2-(((2R,7aS)-2-fluorohexahydro-1H-pyrrolizin-7a-yl)methoxy)-7-(3-(methoxymethoxy)naphthalen-1-yl)pyrido[4,3-d]pyrimidin-4-yl)-3-azabicyclo[3.2.1]octan-6-ol: To a solution of 8-fluoro-2-(((2R,7aS)-2-fluorohexahydro-1H-pyrrolizin-7a-yl)methoxy)-7-(3-(methoxymethoxy)naphthalen-1-yl)-4-(2,2,2-trifluoroethoxy)pyrido[4,3-d]pyrimidine (640 mg, 1.0 equiv) and (1R,5R,6R)-3-azabicyclo[3.2.1]octan-6-ol (266 mg, 1.5 equiv, HCl) in DMF (1.92 mL) were added DIPEA (700 mg, 5.0 equiv) and 4 Å MS (256 mg). The reaction was stirred at 60° C. for 12 hours. The mixture was filtered, concentrated and purified by reversed-phase HPLC [0.1% FA condition] to afford the title compound (480 mg, 71% yield) as a yellow solid. LCMS (ESI, M+1): m/z=618.5.

Step C. (1R,5R,6R)-3-(7-(4-bromo-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of (1R,5R,6R)-3-(8-fluoro-2-(((2R,7aS)-2-fluorohexahydro-1H-pyrrolizin-7a-yl)methoxy)-7-(3-(methoxymethoxy)naphthalen-1-yl)pyrido[4,3-d]pyrimidin-4-yl)-3-azabicyclo[3.2.1]octan-6-ol (100 mg, 1.0 equiv) in THF (2 mL) was added 1,3-dibromo-5,5-dimethyl-imidazolidine-2,4-dione (55.5 mg, 1.2 equiv). The reaction was stirred at 20° C. for 2 hours. The reaction mixture was diluted with H₂O (15 mL) and extracted with EtOAc (15 mL×3). The combined organic layers were washed with brine (20 mL×1), dried over Na₂SO₄, filtered, concentrated and purified with prep-HPLC [column: Phenomenex luna C18 150*25 mm*10 μm; mobile phase: [water (FA)-ACN]; B %: 24%-54%, 10 min] to afford the title compound (22 mg, 30.6 μmol, 18% yield, 97% purity) as a white solid; LCMS (ESI, M+1): m/z=696.2.

Step D. (1R,5R,6R)-3-(7-(4-bromo-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: A solution of (1R,5R,6R)-3-(7-(4-bromo-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (22 mg, 31.6 μmol, 1.0 equiv) in HCl/MeOH (2 mL) was stirred at 0° C. for 0.5 hour. The mixture was concentrated and dissolved in MeCN (5 mL). The pH was adjusted to 7 with solid NaHCO₃. The mixture was filtered, concentrated and purified with prep-HPLC [column: Phenomenex Synergi C18 150*25 mm*10 μm; mobile phase: [water (FA)-ACN]; B %: 13%-43%, 10 min] to afford the title compound (8 mg, 11.3 μmol, 35% yield, 99% purity, 0.15 FA) as a yellow solid; ¹H NMR (400 MHz, DMSO-d₆) δ=10.86 (br s, 1H), 9.43 (s, 1H), 8.16 (d, J=8.4 Hz, 1H), 7.72-7.53 (m, 2H), 7.45 (s, 1H), 7.41-7.33 (m, 1H), 5.57-5.30 (m, 1H), 4.87-4.70 (m, 2H), 4.63 (br d, J=11.6 Hz, 1H), 4.46-4.22 (m, 2H), 4.17 (br dd, J=5.2, 10.0 Hz, 1H), 3.74 (br d, J=12.0 Hz, 1H), 3.38 (br d, J=12.8 Hz, 1H), 3.16 (br s, 2H), 3.13-2.96 (m, 1H), 2.89-2.72 (m, 1H), 2.39-2.21 (m, 3H), 2.20-1.87 (m, 6H), 1.78 (br d, J=10.4 Hz, 1H), 1.72-1.60 (m, 1H), 1.25 (br d, J=14.0 Hz, 1H); LCMS (ESI, M+3): m/z=652.0, 654.0.

### Example 782

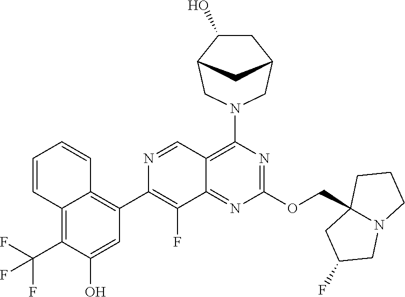 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1C(F)(F)F]

### (1R,5R,6R)-3-(8-fluoro-2-(((2R,7aS)-2-fluorohexahydro-1H-pyrrolizin-7a-yl)methoxy)-7-(3-hydroxy-4-(trifluoromethyl)naphthalen-1-yl)pyrido[4,3-d]pyrimidin-4-yl)-3-azabicyclo[3.2.1]octan-6-ol

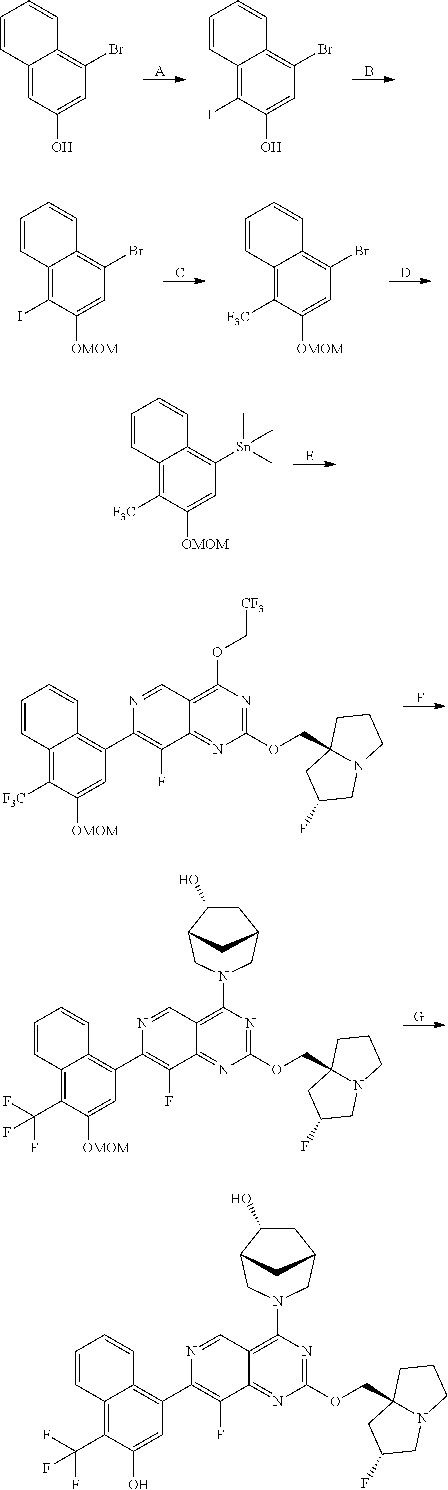

Step A. 4-bromo-1-iodonaphthalen-2-ol: To a solution of 4-bromonaphthalen-2-ol (100 mg, 448.30 μmol, 1 eq) in acetic acid (1.0 mL) was added NIS (151 mg, 1.5 equiv). The mixture was stirred at 25° C. for 3 hours under N₂ atmosphere. The mixture was poured into ice water (10 mL) and extracted with ethyl acetate (3×30 mL). The combined organic layers were washed with brine (50 mL), dried over Na₂SO₄, concentrated and purified by reversed phase flash chromatography [water (0.1% formic acid)/acetonitrile) 0%-100%] to afford 4-bromo-1-iodonaphthalen-2-ol (50.0 mg, 32% yield) as a brown solid; ¹H NMR (400 MHZ, CHLOROFORM-d) δ=7.80 (d, J=8.4 Hz, 1H), 7.61 (d, J=8.4 Hz, 1H), 7.23 (dt, J=1.2, 7.6 Hz, 1H), 7.16-7.10 (m, 1H), 5.43 (s, 1H).

Step B. 4-bromo-1-iodo-2-(methoxymethoxy)naphthalene: To a solution of 4-bromo-1-iodo-naphthalen-2-ol (100 mg, 1.0 equiv) and N-ethyl-N-isopropylpropan-2-amine (111 mg, 3.0 equiv) in dichloromethane (2.0 mL) was added bromo(methoxy) methane (71.6 mg, 2.0 equiv). The mixture was stirred at 25° C. for 1 hour. The mixture was poured into ice water (10 mL) and extracted with ethyl acetate (3×30 mL). The organic layers were washed with brine (50 mL), dried over Na₂SO₄, concentrated and purified by column chromatography (SiO₂, Petroleum ether/Ethyl acetate 50:1 to 5:1) to afford the title compound (100 mg, 89% yield) as a yellow solid; ¹H NMR (400 MHz, CHLOROFORM-d) δ=8.28-8.13 (m, 2H), 7.74 (s, 1H), 7.64-7.46 (m, 2H), 5.36 (s, 2H), 3.59 (s, 3H).

Step C. 4-bromo-2-(methoxymethoxy)-1-(trifluoromethyl)naphthalene: To a solution of 4-bromo-1-iodo-2-(methoxymethoxy)naphthalene (100 mg, 1.0 equiv) and methyl 2,2-difluoro-2-fluorosulfonyl-acetate (978 mg, 20 equiv) in N,N-dimethylacetamide (2.0 mL) was added CuI (485 mg, 10 equiv). The reaction mixture was stirred at 90° C. for 18 hours. The mixture was poured into ice water (10 mL) and extracted with ethyl acetate (3×30 mL). The organic layers were washed with brine (50 mL), dried over Na₂SO₄, concentrated and purified with prep-TLC (SiO₂, Petroleum ether/Ethyl acetate 10:1) to afford the title compound (50.0 mg, 59% yield) as a yellow oil; ¹H NMR (400 MHZ, CHLOROFORM-d) δ=8.27 (d, J=8.4 Hz, 1H), 8.23 (br d, J=8.8 Hz, 1H), 7.85 (s, 1H), 7.64-7.59 (m, 1H), 7.57-7.50 (m, 1H), 5.32 (s, 2H), 3.57 (s, 3H).

Step D. (3-(methoxymethoxy)-4-(trifluoromethyl)naphthalen-1-yl)trimethylstannane: To a mixture of 4-bromo-2-(methoxymethoxy)-1-(trifluoromethyl)naphthalene (50 mg, 1.0 equiv) and Pd(PPh₃)₄ (17.2 mg, 0.10 equiv) in toluene (2.0 mL) was added trimethyl(trimethylstannyl) stannane (0.990 g. 20 equiv) at 25° C. under N₂. The reaction mixture was stirred at 90° C. for 2.5 hours under N₂ atmosphere. The mixture was poured into ice water (5 mL) and extracted with ethyl acetate (3×10 mL). The organic layers were washed with brine (20 mL), dried over Na₂SO₄, concentrated and purified with column chromatography by prep-TLC (SiO₂, DCM:PE 0:1) to afford the title compound (2.00 mg, 3.2% yield) as a yellow oil. ¹H NMR (400 MHZ, CHLOROFORM-d) δ=8.41-8.21 (m, 1H), 7.78 (br d, J=8.0 Hz, 1H), 7.62 (s, 1H), 7.60-7.53 (m, 1H), 7.51-7.43 (m, 1H), 5.58-5.21 (m, 2H), 3.59 (s, 3H), 0.73-0.32 (m, 9H)

Step E. 8-fluoro-2-(((2R,7aS)-2-fluorohexahydro-1H-pyrrolizin-7a-yl)methoxy)-7-(3-(methoxymethoxy)-4-(trifluoromethyl)naphthalen-1-yl)-4-(2,2,2-trifluoroethoxy)pyrido[4,3-d]pyrimidine: To a mixture of [3-(methoxymethoxy)-4-(trifluoromethyl)-1-naphthyl]-trimethyl-stannane (477 mg, 1.0 equiv), 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 1.0 equiv), CuI (86.8 mg, 0.40 equiv) in toluene (20 mL) were added Pd(dppf)Cl₂ (83.4 mg, 0.10 equiv) and (+)-2,2′-bis(diphenylposphino)-1,1′-binaphthalene (142 mg, 228 μmol, 0.20 equiv). The reaction mixture was stirred at 90° C. for 12 hours under N₂ atmosphere. The mixture was poured into ice water (10 mL) and extracted with ethyl acetate (3×20 mL). The organic layers were washed with brine (20 mL), dried over Na₂SO₄, concentrated and purified with reversed phase flash chromatography [water (0.1% formic acid)/acetonitrile) 0% to 100%] to afford the title compound (170 mg, 20% yield) as a yellow solid. LCMS (ESI, M+1): m/z=659.2

Step F. (1R,5R,6R)-3-(8-fluoro-2-(((2R,7aS)-2-fluorohexahydro-1H-pyrrolizin-7a-yl)methoxy)-7-(3-(methoxymethoxy)-4-(trifluoromethyl)naphthalen-1-yl)pyrido[4,3-d]pyrimidin-4-yl)-3-azabicyclo[3.2.1]octan-6-ol: To a solution of 8-fluoro-2-(((2R,7aS)-2-fluorotetrahydro-1H-pyrrolizin-7a(5H)-yl)methoxy)-7-(3-(methoxymethoxy)-4-(trifluoromethyl)naphthalen-1-yl)-4-(2,2,2-trifluoroethoxy)pyrido[4,3-d]pyrimidine (150 mg, 1.0 equiv), (1R,5R,6R)-3-azabicyclo[3.2.1]octan-6-ol (57.9 mg, 2.0 equiv) in N,N-dimethylformamide (1.5 mL) was added N-ethyl-N-isopropylpropan-2-amine (147 mg, 5.0 equiv) and 4 Å molecular sieves (100 mg). The reaction mixture was stirred at 40° C. for 36 hours under N₂ atmosphere. The reaction mixture was poured into ice water (10 mL) and extracted with ethyl acetate (3×20 mL). The organic layers were washed with brine (20 mL), dried over Na₂SO₄, concentrated and purified with reversed phase flash chromatography [water (0.1% formic acid)/acetonitrile) 0% to 100%] to afford the title compound (140 mg, 90% yield) as a yellow solid. LCMS (ESI, M+1): m/z=686.1

Step G. (1R,5R,6R)-3-(8-fluoro-2-(((2R,7aS)-2-fluorohexahydro-1H-pyrrolizin-7a-yl)methoxy)-7-(3-hydroxy-4-(trifluoromethyl)naphthalen-1-yl)pyrido[4,3-d]pyrimidin-4-yl)-3-azabicyclo[3.2.1]octan-6-ol: A mixture of (1R,5R,6R)-3-(8-fluoro-2-(((2R,7aS)-2-fluorotetrahydro-1H-pyrrolizin-7a(5H)-yl)methoxy)-7-(3-(methoxymethoxy)-4-(trifluoromethyl)naphthalen-1-yl)pyrido[4,3-d]pyrimidin-4-yl)-3-azabicyclo[3.2.1]octan-6-ol (110 mg, 160.42 μmol, 1 eq) and HCl·MeOH (4 M, 1.10 mL, 27.43 eq) in MeOH (1 mL) was stirred at 25° C. for 10 min under N₂ atmosphere. The mixture was poured into ice water (10 mL) and extracted with ethyl acetate (3×20 mL). The combined organic layers were washed with brine (20 mL), dried over Na₂SO₄, concentrated and purified with prep-HPLC [column: Unisil 3-100 C18 Ultra 150×50 mm×3 μm; A: water (FA); B: ACN, B %: 20%-50% over 7 min] to afford the title compound (36.4 mg, 35% yield, 0.74 HCOOH salt) as a white solid. ¹H NMR (400 MHZ, CHLOROFORM-d) δ=9.09 (s, 1H), 8.16 (br d, J=8.4 Hz, 1H), 7.60 (d, J=8.4 Hz, 1H), 7.53 (t, J=8.0 Hz, 1H), 7.44 (s, 1H), 7.35-7.28 (m, 1H), 5.50-5.30 (m, 1H), 5.12 (br d, J=12.0 Hz, 1H), 4.85 (br d, J=10.8 Hz, 1H), 4.39 (br d, J=11.2 Hz, 1H), 4.36-4.26 (m, 2H), 3.89 (br dd, J=1.6, 14.0 Hz, 1H), 3.83-3.60 (m, 3H), 3.42-3.28 (m, 2H), 3.20-3.08 (m, 1H), 2.70-2.45 (m, 1H), 2.44-2.35 (m, 1H), 2.33-2.25 (m, 2H), 2.25-2.06 (m, 4H), 1.83-1.69 (m, 2H), 1.21 (br d, J=14.8 Hz, 1H). LCMS (ESI, M+1): m/z=642.2.

### Example 783

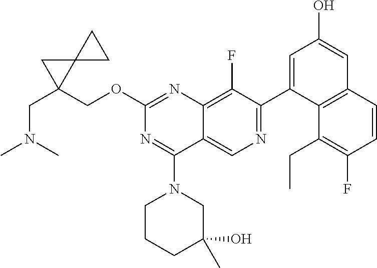 [SMILES: CCc1c(F)ccc2cc(O)cc(-c3ncc4c(N5CCC[C@@](C)(O)C5)nc(OCC5(CN(C)C)CC56CC6)nc4c3F)c12]

### (3R)-1-(2-((1-((dimethylamino)methyl)spiro[2.2]pentan-1-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

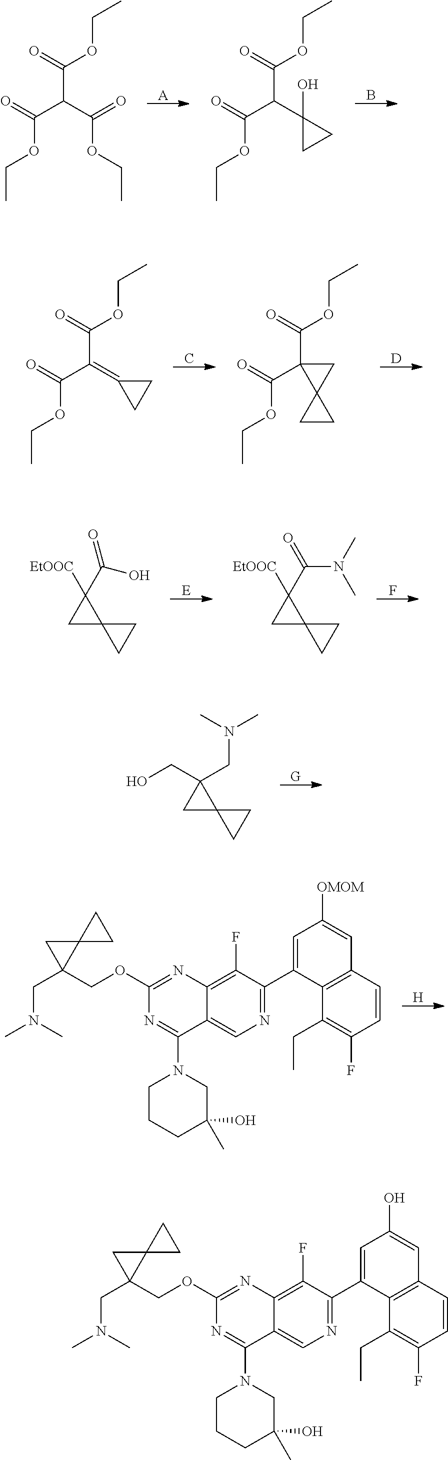

Step A. diethyl 2-(1-hydroxycyclopropyl) malonate: To a mixture of triethyl methanetricarboxylate (25.0 g, 1.0 equiv) and Ti(Oi-Pr)₄ (12.2 g, 0.4 equiv) in THF (200 mL) was added EtMgBr (3 M, 7.0 equiv). The reaction mixture was stirred at 20° C. for 1 hour under N₂ atmosphere. The reaction mixture was quenched with aqueous HCl solution (4 M, 500 mL), extracted with EtOAc (500 mL), washed with brine (500 mL×2), dried over Na₂SO₄, concentrated and purified with column chromatography [SiO₂, petroleum ether/ethyl acetate 1:0 to 3:1] to afford the title compound (6.1 g, 26% yield) as a yellow oil. ¹H NMR (400 MHZ, CHLOROFORM-d) δ=4.26-4.17 (m, 4H), 3.86-3.69 (m, 1H), 2.94 (s, 1H), 1.24 (t, J=7.2 Hz, 6H), 0.92-0.83 (m, 2H), 0.65-0.58 (m, 2H).

Step B. diethyl 2-cyclopropylidenemalonate: To a mixture of diethyl 2-(1-hydroxycyclopropyl) malonate (6.0 g, 1.0 equiv) and TEA (15.7 g, 7.0 equiv) in DCM (60.0 mL) was added methylsulfonyl methanesulfonate (7.7 g, 2.0 equiv). The reaction mixture was stirred at 25° C. for 1 hour under N₂ atmosphere. The mixture was poured into ice-water (50 mL), extracted with DCM (20 mL×2), washed with brine (50 mL), dried over Na₂SO₄, concentrated and purified with column chromatography [SiO₂, petroleum ether/ethyl acetate 1:0 to 4:1] to afford the title compound (1.9 g, 43% yield) as a yellow oil. ¹H NMR (400 MHZ, CHLOROFORM-d) δ=4.31-4.28 (m, 4H), 1.55 (s, 4H), 1.35-1.31 (m, 6H).

Step C. diethyl spiro[2.2]pentane-1,1-dicarboxylate: To a solution of trimethylsulfoxonium iodide (3.2 g, 1.5 equiv) in DMSO (20 mL) was added NaH (575 mg, 60% purity, 1.5 equiv). The mixture was stirred at 0° C. for 0.5 hour. Then diethyl 2-cyclopropylidenemalonate (1.9 g, 1.0 equiv) was added. The mixture was stirred at 25° C. for 12 hours. The reaction mixture was quenched with aqueous NH₄Cl solution (30 mL) at 0° C., diluted with water (30 mL) and extracted with EtOAc (30 mL×3). The combined organic layers were washed with brine (50 mL×2), dried over anhydrous Na₂SO₄, concentrated and purified with column chromatography [SiO₂, petroleum ether/ethyl acetate 1:0 to 4:1] to afford the title compound (680 mg, 33% yield) as a yellow oil. ¹H NMR (400 MHZ, xCHLOROFORM-d) δ=4.29-4.18 (m, 4H), 1.91 (s, 2H), 1.31-1.26 (m, 6H), 1.10-1.01 (m, 4H).

Step D. 1-(ethoxycarbonyl)spiro[2.2]pentane-1-carboxylic acid: To a mixture of diethyl spiro[2.2]pentane-1,1-dicarboxylate (630 mg, 1.0 equiv) in EtOH (6 mL) was added aqueous NaOH solution (1 M, 1.1 equiv). The reaction mixture was stirred at 25° C. for 1 hour under N₂ atmosphere. The reaction mixture was adjusted to pH=2 with 1M HCl and extracted with EtOAc (10 mL). The combined layers were washed with brine (10 mL), dried over anhydrous Na₂SO₄ and concentrated to afford the title compound (480 mg, 88% yield) as a yellow oil. ¹H NMR (400 MHZ, CHLOROFORM-d) δ=4.40-4.20 (m, 2H), 2.35 (d, J=3.2 Hz, 1H), 2.26 (d, J=3.2 Hz, 1H), 1.35-1.29 (m, 3H), 1.22-1.01 (m, 4H).

Step E. ethyl 1-(dimethylcarbamoyl)spiro[2.2]pentane-1-carboxylate: To a mixture of 1-(ethoxycarbonyl)spiro[2.2]pentane-1-carboxylic acid (200 mg, 1.0 equiv), N-methylmethanamine (177 mg, 2.0 equiv, HCl salt) and HATU (826 mg, 2.0 equiv) in DCM (3 mL) was added DIEA (702 mg, 5.0 equiv). The mixture was stirred at 25° C. for 12 hours. The mixture was poured into ice-water (3 mL), extracted with EtOAc (6 mL), washed with brine (3 mL), dried over anhydrous Na₂SO₄, concentrated and purified with column chromatography [SiO₂, petroleum ether/ethyl acetate 10:1 to 1:1] to afford the title compound (200 mg, 87% yield) as a yellow oil. ¹H NMR (400 MHZ, CHLOROFORM-d) δ=4.19 (q, J=7.2 Hz, 2H), 2.98 (s, 3H), 2.83 (s, 3H), 1.89-1.80 (m, 2H), 1.28-1.22 (m, 3H), 1.15-0.88 (m, 4H).

Step F. (1-((dimethylamino)methyl)spiro[2.2]pentan-1-yl)methanol: To a mixture of ethyl 1-(dimethylcarbamoyl)spiro[2.2]pentane-1-carboxylate (150 mg, 1.0 equiv) in THF (1 mL) was added LiAlH₄ (135 mg, 5.0 equiv) at 0° C. The reaction mixture was stirred at 25° C. for 1 hour. The mixture was added sat. Na₂SO₄ (3 mL) at 0° C., dried over anhydrous Na₂SO₄ and concentrated to afford the title compound (100 mg, 91% yield) as a yellow oil.

Step G. (3R)-1-(2-((1-((dimethylamino)methyl)spiro[2.2]pentan-1-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (395 mg, 1.0 equiv) and (1-((dimethylamino)methyl)spiro[2.2]pentan-1-yl)methanol (180 mg, 1.0 equiv) in THF (2 mL) was added NaHMDS (1 M, 897 μL, 1.5 equiv). The reaction mixture stirred at −10° C. for 1 hour under N₂ atmosphere. The reaction mixture was quenched by addition of sat. aqueous NH₄Cl solution (10 mL) at 0° C., extracted with EtOAc (20 mL), washed with brine (10 mL×2), dried over anhydrous Na₂SO₄, concentrated and purified with prep-HPLC [column: Phenomenex luna C18 150×25 mm×10 μm; mobile phase: [water (FA)-ACN]; B %: 23%-53%, 10 minutes] to afford the title compound (38 mg, 10% yield, HCOOH salt) as a white solid. ¹H NMR (400 MHZ, DMSO-d₆) δ=9.23 (s, 1H), 7.90 (dd, J=6.0, 9.2 Hz, 1H), 7.68 (d, J=2.4 Hz, 1H), 7.44 (t, J=9.6 Hz, 1H), 7.24 (s, 1H), 5.35 (s, 2H), 4.92-4.65 (m, 1H), 4.49 (br d, J=9.6 Hz, 1H), 4.42-4.16 (m, 2H), 4.12-3.92 (m, 1H), 2.67 (d, J=1.6 Hz, 1H), 2.33 (br s, 2H), 2.28-2.09 (m, 9H), 2.07-1.92 (m, 2H), 1.82-1.42 (m, 4H), 1.27-1.06 (m, 4H), 0.93-0.41 (m, 9H); LCMS (ESI, M+1): m/z=648.1.

Step H. (3R)-1-(2-((1-((dimethylamino)methyl)spiro[2.2]pentan-1-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: A solution of (3R)-1-(2-((1-((dimethylamino)methyl)spiro[2.2]pentan-1-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (38.0 mg, 1.0 equiv) in HCl·MeOH (4 M, 1 mL, 68.2 equiv) was stirred at 0° C. for 1 hour. The mixture was poured into sat. aqueous NaHCO₃ solution (5 mL), extracted with EtOAc (5 mL×2), washed with brine (5 mL), dried over anhydrous Na₂SO₄, concentrated and purified with prep-HPLC [column: Phenomenex luna C18 150×25 mm×10 μm; mobile phase: [water (FA)-ACN]; B %: 18%-48%, 10 minutes] to afford the title compound (10.7 mg, 29% yield, 0.78 HCOOH salt) as a white solid. ¹H NMR (400 MHZ, METHANOL-d₄) δ=9.26 (dd, J=4.2, 6.9 Hz, 1H), 8.37 (s, 1H), 7.70 (dd, J=6.0, 8.8 Hz, 1H), 7.33 (d, J=2.4 Hz, 1H), 7.27 (t, J=9.2 Hz, 1H), 7.08 (s, 1H), 4.78-4.52 (m, 3H), 4.46-4.26 (m, 2H), 3.74-3.54 (m, 1H), 3.52-3.35 (m, 2H), 2.88-2.63 (m, 7H), 2.62-2.38 (m, 1H), 2.35-2.06 (m, 2H), 1.98-1.69 (m, 3H), 1.42-1.19 (m, 5H), 1.14-0.71 (m, 7H); LCMS (ESI, M+1): m/z=604.4.

### Example 784

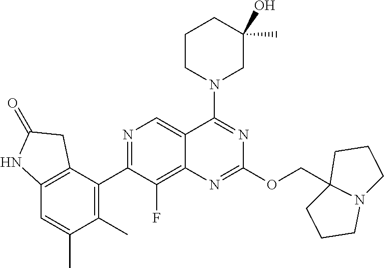 [SMILES: Cc1cc2c(c(-c3ncc4c(N5CCC[C@@](C)(O)C5)nc(OCC56CCCN5CCC6)nc4c3F)c1C)CC(=O)N2]

### 4-(8-fluoro-2-((hexahydro-1H-pyrrolizin-7a-yl)methoxy)-4-((R)-3-hydroxy-3-methylpiperidin-1-yl)pyrido[4,3-d]pyrimidin-7-yl)-5,6-dimethylindolin-2-one

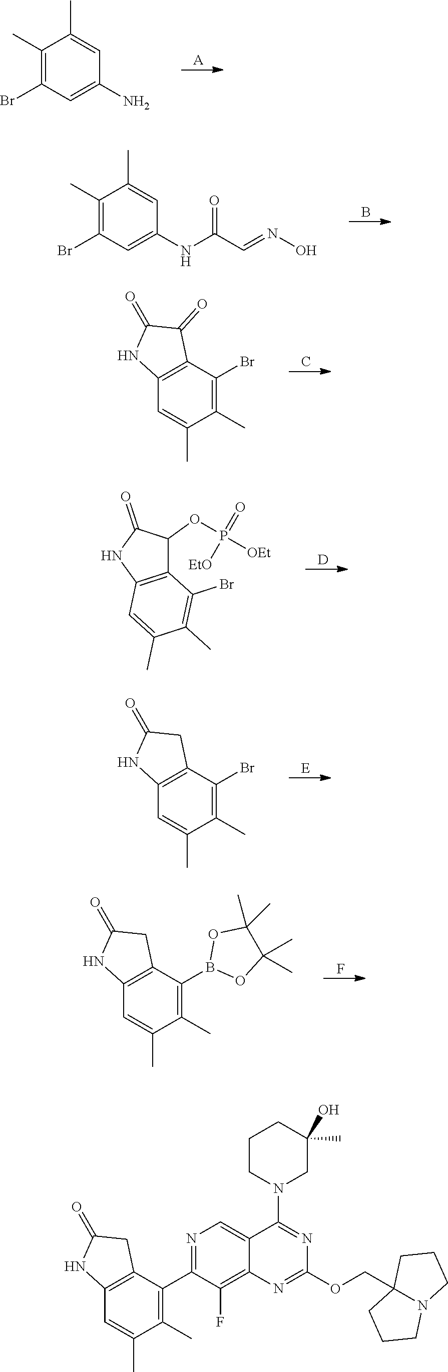

Step A. (E)-N-(3-bromo-4,5-dimethylphenyl)-2-(hydroxyimino)acetamide: A mixture of 3-bromo-4,5-dimethyl-aniline (10.0 g, 1.0 equiv), 2,2,2-trichloroethane-1,1-diol (9.92 g, 1.2 equiv), Na₂SO₄ (28.4 g, 20.3 mL, 4.0 equiv) and NH₂OH·HCl (5.21 g, 1.5 equiv) in ethyl alcohol (200 mL) and H₂O (200 mL) was degassed and stirred at 80° C. for 12 hours under N₂ atmosphere. The reaction was extracted with ethyl acetate (3× 500 mL). The combined organic layers were washed with brine (500 mL), dried over Na₂SO₄, concentrated and purified with reversed phase flash chromatography [C 18, FA condition] to afford the title compound (7.50 g, crude) as a yellow solid.

Step B. 4-bromo-5,6-dimethylindoline-2,3-dione: A mixture of (E)-N-(3-bromo-4,5-dimethylphenyl)-2-(hydroxyimino)acetamide (8.00 g, 1.0 equiv) and H₂SO₄ (18.1 M, 20 equiv) in H₂O (32 mL) was stirred at 80° C. for 12 hours under N₂ atmosphere. The mixture was cooled to room temperature. The pH was adjusted to 7 with ammonium hydroxide. The mixture was extracted with ethyl acetate (3× 500 mL). The combined organic layers were washed with brine (500 mL), dried over Na₂SO₄, concentrated and purified with reversed phase flash chromatography [C 18, 0.1% FA] to afford the title compound (2.00 g, 27% yield) as a yellow solid; LCMS (ESI, M+1): m/z=254.0.

Step C. 4-bromo-5,6-dimethyl-2-oxoindolin-3-yl diethyl phosphate: To a solution of 4-bromo-5,6-dimethyl-indoline-2,3-dione (270 mg, 1.0 equiv), 1-ethoxyphosphonoyloxyethane (176 mg, 1.2 equiv) in acetonitrile (2.0 mL) was added Na₂CO₃ (11.3 mg, 0.10 equiv). The reaction mixture was stirred at 60° C. for 6 hours under N₂ atmosphere. The mixture was diluted with water (20 mL) and extracted with dichloromethane (3×30 mL). The organic layers were washed with brine (500 mL), dried over Na₂SO₄, concentrated and purified by trituration to afford the title compound (290 mg, 70% yield) as a yellow solid; LCMS (ESI, M+1): m/z=392.0.

Step D. 4-bromo-5,6-dimethylindolin-2-one: To a solution of (4-bromo-5,6-dimethyl-2-oxo-indolin-3-yl) diethyl phosphate (200 mg, 1.0 equiv) in acetonitrile (1.0 mL) and H₂O (0.2 mL) was added hydroiodic acid (572 mg, 57% purity, 5.0 equiv). The reaction mixture was stirred at 50° C. for 1 hour under N₂ atmosphere. The reaction mixture was diluted with water (10 mL) and extracted with ethyl acetate (3×20 mL). The organic layers were washed with brine (20 mL), dried over Na₂SO₄, concentrated and purified by trituration to afford the title compound (100 mg, 82% yield) as a yellow solid; LCMS (ESI, M+1): m/z=240.0

Step E. 5,6-dimethyl-4-(4,4,5,5-tetramethyl-1,3,2-dioxaborolan-2-yl) indolin-2-one: To a solution of 4-bromo-5,6-dimethyl-indolin-2-one (500 mg, 1.0 equiv), 4,4,5,5-tetramethyl-2-(4,4,5,5-tetramethyl-1,3,2-dioxaborolan-2-yl)-1,3,2-dioxaborolane (1.06 g, 2.0 equiv), KOAc (613 mg, 3.0 equiv) in dioxane (10 mL) was added PCy3 Pd G2 (123 mg, 0.1 equiv). The reaction mixture was stirred at 80° C. for 12 hours under N₂ atmosphere. The mixture was diluted with water (10 mL) and extracted with ethyl acetate (3×20 mL). The organic layers were washed with brine (20 mL), dried over Na₂SO₄, concentrated and purified by reversed phase flash chromatography [C 18, water (FA); B: ACN, B %: 0%-75%, 25 min] to afford the title compound (100 mg, 82% yield) as a yellow solid; LCMS (ESI, M+1): m/z=288.1

Step F. 4-(8-fluoro-2-((hexahydro-1H-pyrrolizin-7a-yl)methoxy)-4-((R)-3-hydroxy-3-methylpiperidin-1-yl)pyrido[4,3-d]pyrimidin-7-yl)-5,6-dimethylindolin-2-one: To a solution of 5,6-dimethyl-4-(4,4,5,5-tetramethyl-1,3,2-dioxaborolan-2-yl) indolin-2-one (160 mg, 1.5 equiv), (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (162 mg, 1.0 equiv) in toluene (3 mL) were added CataCXium A Pd G3 (27.1 mg, 0.10 equiv) and K₃PO₄ (1.5 M in H₂O, 3.0 equiv). The reaction mixture was stirred at 100° C. for 12 hours under N₂ atmosphere. The reaction mixture was diluted with water (5 mL) and extracted with ethyl acetate (3×30 mL). The organic layers were washed with brine (50 mL), dried over Na₂SO₄, concentrated and purified with prep-HPLC [column: Welch Ultimate XB-SiOH 250×50×10 μm; A: Hexane; B: EtOH, B %: 5%-45% over 15 min] and prep-HPLC [column: Phenomenex luna C18 150×25 mm×10 μm; A: water (FA); B: ACN, B %: 10%-40% over 10 min] to afford the title compound (22.5 mg, 11% yield, 0.84 HCOOH salt) as a yellow solid; ¹H NMR (400 MHZ, METHANOL-d₄) δ=9.29 (d, J=8.8 Hz, 1H), 6.89 (s, 1H), 4.60 (s, 3H), 4.33 (br d, J=13.2 Hz, 1H), 3.74-3.52 (m, 3H), 3.51-3.32 (m, 2H), 3.27-3.10 (m, 2H), 2.58-2.33 (m, 4H), 2.32-2.25 (m, 2H), 2.24-2.08 (m, 6H), 2.08-2.00 (m, 4H), 1.94-1.69 (m, 3H), 1.30 (d, J=1.6 Hz, 3H). LCMS (ESI, M+1): m/z=561.4

### Example 785

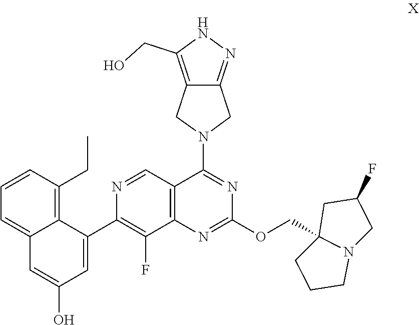 [SMILES: CCc1cccc2cc(O)cc(-c3ncc4c(N5Cc6n[nH]c(CO)c6C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(3-(hydroxymethyl) pyrrolo[3,4-c]pyrazol-5 (2H,4H,6H)-yl)pyrido[4,3-d]pyrimidin-7-yl)naphthalen-2-ol

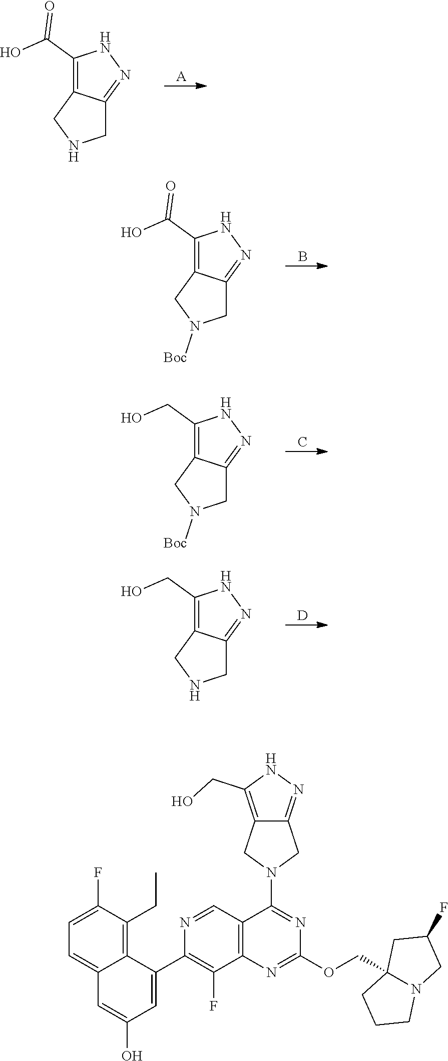

Step A: 5-(tert-butoxycarbonyl)-2,4,5,6-tetrahydropyrrolo[3,4-c]pyrazole-3-carboxylic acid: To a solution of 2,4,5,6-tetrahydropyrrolo[3,4-c]pyrazole-3-carboxylic acid (40.0 mg, 1.0 equiv) and TEA (79.3 mg, 3.0 equiv) in DCM (2.00 mL) was added Boc₂O (85.5 mg, 1.5 equiv). The mixture was stirred at 20° C. for 1 hour. The reaction mixture was concentrated and purified with prep-HPLC [FA condition; column: Phenomenex luna C18 150×25 mm×10 um; mobile phase: [water (FA)-ACN]; B %: 21%-51%, 10 minutes] to afford the title compound (35.0 mg, 53% yield) as a white solid. LCMS (ESI, M+1): m/z=198.0.

Step B: tert-butyl 3-(hydroxymethyl)-4,6-dihydropyrrolo[3,4-c]pyrazole-5 (2H)-carboxylate: To a solution of 5-(tert-butoxycarbonyl)-2,4,5,6-tetrahydropyrrolo[3,4-c]pyrazole-3-carboxylic acid (100 mg, 1.0 equiv) in THF (20.0 mL) was slowly added BH₃·Me₂S (10 M, 197 μL, 5.0 equiv). The mixture was stirred at 20° C. for 12 hours. The reaction mixture was quenched with methanol (1.0 mL) at 0° C., diluted with water (20.0 mL), extracted with ethyl acetate (20.0 mL×2), washed with brine (20.0 mL), dried over anhydrous sodium sulfate, filtered and purified with prep-HPLC [TFA condition; column: 3_Phenomenex Luna C18 75×30 mm×3 um; mobile phase: [water (TFA)-ACN]; B %: 19%-39%, 9 minutes] to afford the title compound (60.0 mg, 64% yield) as a white solid. LCMS (ESI, M+1): m/z=240.1.

Step C: (2,4,5,6-tetrahydropyrrolo[3,4-c]pyrazol-3-yl)methanol: To a solution of tert-butyl 3-(hydroxymethyl)-4,6-dihydropyrrolo[3,4-c]pyrazole-5 (2H)-carboxylate (50.0 mg, 1.0 equiv) in DCM (0.75 mL) was added TFA (0.25 mL). The reaction mixture was stirred at 20° C. for 0.5 hour. The mixture was concentrated to afford the title compound (50.0 mg, crude) as a yellow oil. LCMS (ESI, M+1): m/z=140.1.

Step D: 5-ethyl-6-fluoro-4-(8-fluoro-2-(((2R,7aS)-2-fluorohexahydro-1H-pyrrolizin-7a-yl)methoxy)-4-(3-(hydroxymethyl) pyrrolo[3,4-c]pyrazol-5 (2H,4H,6H)-yl)pyrido[4,3-d]pyrimidin-7-yl)naphthalen-2-ol: To a solution of (2,4,5,6-tetrahydropyrrolo[3,4-c]pyrazol-3-yl)methanol (50.0 mg, 3.0 equiv), K₃PO₄ (254 mg, 10.0 equiv) and 4 Å molecular sieves (100 mg) in DMF (5.0 mL) was added 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (71.0 mg, 1.0 equiv). The mixture was stirred at 60° C. for 2 hours. The reaction mixture was diluted with water (50.0 mL), extracted with ethyl acetate (50.0 mL×2), washed with brine (50.0 mL), dried over anhydrous sodium sulfate, concentrated and purified with prep-HPLC [basic condition; column: Waters Xbridge 150×25 mm×5 μm; mobile phase: [water (ammonia hydroxide v/v)-ACN]; B %: 27%-57%, 9 minutes] to afford the title compound (12.5 mg, 16% yield) as a white solid. ¹H NMR (400 MHZ, CD₃OD) δ=9.43 (s, 1H), 7.67 (dd, J=6.0, 8.8 Hz, 1H), 7.30 (d, J=2.8 Hz, 1H), 7.25 (t, J=9.2 Hz, 1H), 7.07 (s, 1H), 5.41-5.07 (m, 5H), 4.71 (s, 2H), 4.66-4.51 (m, 1H), 4.47-4.25 (m, 2H), 3.26-3.18 (m, 2H), 3.08-2.98 (m, 1H), 2.53-1.90 (m, 8H), 0.80 (br t, J=7.2 Hz, 3H); LCMS (ESI, M+1): m/z=632.3.

### Example 786

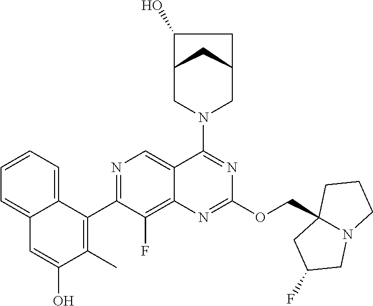 [SMILES: Cc1c(O)cc2ccccc2c1-c1ncc2c(N3C[C@@H]4C[C@H](C3)[C@H](O)C4)nc(OC[C@@]34CCCN3C[C@H](F)C4)nc2c1F]

### (1R,5R,6R)-3-(8-fluoro-2-(((2R,7aS)-2-fluorohexahydro-1H-pyrrolizin-7a-yl)methoxy)-7-(3-hydroxy-2-methylnaphthalen-1-yl)pyrido[4,3-d]pyrimidin-4-yl)-3-azabicyclo[3.2.1]octan-6-ol

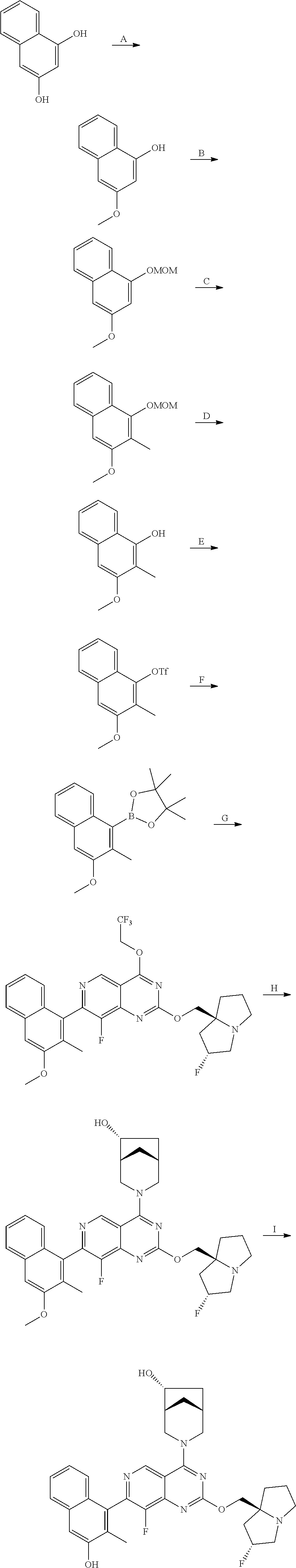

Step A. 3-methoxynaphthalen-1-ol: To a solution of naphthalene-1,3-diol (50.0 g, 1.0 equiv) in methanol (750 mL) was added HCl·MeOH (4 M, 750 mL) dropwise at 0° C. The mixture was stirred at 15° C. for 36 hours. The pH of the mixture was adjusted to 7 with 3M NaOH aqueous at 0° C. The mixture was concentrated, diluted with water (1 L) and extracted with ethyl acetate (3× 500 mL). The combined organic layer dried over Na₂SO₄, concentrated and purified with column chromatography [SiO₂, petroleum ether/ethyl acetate 50:1 to 10:1] to afford the title compound (38.0 g, 70% yield) as a red solid; ¹H NMR (400 MHz, DMSO-d6) δ=10.20 (s, 1H), 8.00 (d, J=8.4 Hz, 1H), 7.69 (d, J=8.4 Hz, 1H), 7.45-7.36 (m, 1H), 7.30-7.19 (m, 1H), 6.77 (d, J=2.0 Hz, 1H), 6.52 (d, J=2.4 Hz, 1H), 3.81 (s, 3H).

Step B. 3-methoxy-1-(methoxymethoxy)naphthalene: To a solution of 3-methoxynaphthalen-1-ol (12.5 g, 1.0 equiv) and N-ethyl-N,N-diisopropylamine (21.7 g, 2.3 equiv) in tetrahydrofuran (100 mL) was added bromo(methoxy) methane (10.5 g, 1.2 equiv) dropwise at 0° C. The reaction mixture was stirred at 0° C. for 1 hour. The mixture was quenched with saturated NaHCO₃ aqueous (50 mL) at 0° C. and extracted with ethyl acetate (3× 40 mL). The organic phase was dried over Na₂SO₄, concentrated and purified with column chromatography [SiO₂, petroleum ether/ethyl acetate 100:1 to 20:1] to afford the title compound (44.2 g, 92% yield) as a colorless oil; ¹H NMR (400 MHZ, DMSO-d6) δ=8.06 (d, J=8.4 Hz, 1H), 7.77 (d, J=8.4 Hz, 1H), 7.47 (dt, J=1.2, 7.6 Hz, 1H), 7.33 (ddd, J=1.2, 7.2, 8.4 Hz, 1H), 6.96 (d, J=2.0 Hz, 1H), 6.76 (d, J=2.4 Hz, 1H), 5.40 (s, 2H), 3.85 (s, 3H), 3.45 (s, 3H).

Step C. 3-methoxy-1-(methoxymethoxy)-2-methylnaphthalene: To a solution of 3-methoxy-1-(methoxymethoxy)naphthalene (3.00 g, 1.0 equiv) and N,N,N′,N′-tetramethylethane-1,2-diamine (1.61 g, 1.0 equiv) in tetrahydrofuran (30 mL) was added n-BuLi (2.5 M, 11 mL, 2.0 equiv) over the period of 5 minutes at 0° C. The mixture was stirred at 0° C. for 2 hours. CH₃I (3.90 g, 2.0 equiv) was added slowly over 2 minutes at 0° C. and the mixture was stirred at 0° C. for 0.5 hour. The mixture was quenched with saturated aqueous NH₄Cl (10 mL) at 0° C. and extracted with ethyl acetate (2×10 mL). The organic layer was washed with brine (10 mL), dried over Na₂SO₄, concentrated and purified with column chromatography [SiO₂, Petroleum ether/Ethyl acetate 0:1 to 20:1] to afford the title compound (2.80 g, crude) as a yellow oil; ¹H NMR (400 MHz, METHANOL-d₄) δ=7.95 (d, J=8.4 Hz, 1H), 7.71 (d, J=8.0 Hz, 1H), 7.39-7.33 (m, 1H), 7.33-7.27 (m, 1H), 7.01 (s, 1H), 5.09 (d, J=1.2 Hz, 2H), 3.90 (d, J=1.2 Hz, 3H), 3.61 (d, J=1.2 Hz, 3H), 2.29 (d, J=0.8 Hz, 3H).

Step D. 3-methoxy-2-methylnaphthalen-1-ol: To a solution of 3-methoxy-1-(methoxymethoxy)-2-methylnaphthalene (1.40 g, 1.0 equiv) in ACN (7 mL) was added HCl·dioxane (4 M, 14 mL, 9.3 equiv) at 0° C. The mixture was stirred at 0° C. for 0.5 hours. The mixture was poured into saturated aqueous NaHCO₃ (15 mL) and extracted with ethyl acetate (2× 10 mL). The organic layer was washed with brine (10 mL), dried over Na₂SO₄, concentrated and purified with column chromatography [SiO₂, petroleum ether/ethyl acetate 0:1 to 10:1] to afford the title compound (500 mg, 44% yield) as a yellow oil; ¹H NMR (400 MHZ, METHANOL-d₄) δ=8.05 (d, J=8.4 Hz, 1H), 7.65 (d, J=8.0 Hz, 1H), 7.32 (dt, J=1.2, 7.6 Hz, 1H), 7.27-7.21 (m, 1H), 6.81 (s, 1H), 3.91 (s, 3H), 2.24 (s, 3H); LCMS (ESI, M+1): m/z=189.2.

Step E. 3-methoxy-2-methylnaphthalen-1-yl trifluoromethanesulfonate: To a solution of 3-methoxy-2-methylnaphthalen-1-ol (1.00 g, 1.0 equiv) and N-ethyl-N,N-diisopropylamine (2.06 g, 3.0 equiv) in dichloromethane (8 mL) was added trifluoromethylsulfonic anhydride (2.25 g, 1.5 equiv) at −40° C. The mixture was stirred at −40° C. for 30 minutes. The mixture was diluted with water (10 mL) and extracted with dichloromethane (2×10 mL). The organic layer was washed with brine (10 mL), dried over Na₂SO₄, concentrated and purified with column chromatography [SiO₂, petroleum ether/ethyl acetate 1:0 to 20:1] to afford the title compound (1.50 g crude) as a yellow oil. ¹H NMR (400 MHZ, METHANOL-d₄) δ=7.89 (dd, J=8.0, 14.0 Hz, 2H), 7.55-7.49 (m, 1H), 7.47 (dd, J=1.2, 8.4 Hz, 1H), 7.37 (s, 1H), 4.01 (s, 3H), 2.39 (s, 3H).

Step F. 2-(3-methoxy-2-methylnaphthalen-1-yl)-4,4,5,5-tetramethyl-1,3,2-dioxaborolane: To a solution of 3-methoxy-2-methylnaphthalen-1-yl trifluoromethanesulfonate (700 mg, 1.0 equiv) and 4,4,4′,4′,5,5,5′,5′-octamethyl-2,2′-bi (1,3,2-dioxaborolane) (2.22 g, 4.0 equiv) in dioxane (8 mL) were added potassium acetate (643 mg, 3.0 equiv) and Pd(dppf) C12 (160 mg, 0.1 equiv). The mixture was stirred at 100° C. for 12 hours. The mixture was diluted with water (10 mL) and extracted with ethyl acetate (2×10 mL). The organic layer was washed with brine (10 mL), dried over Na₂SO₄, concentrated and purified with column chromatography [SiO₂, petroleum ether/ethyl acetate 1:0 to 20:1] to afford the title compound (350 mg, 54% yield) as a yellow oil; ¹H NMR (400 MHZ, METHANOL-d₄) δ=7.87 (d, J=8.4 Hz, 1H), 7.71 (d, J=8.0 Hz, 1H), 7.37-7.30 (m, 1H), 7.29-7.22 (m, 1H), 7.21 (s, 1H), 3.93 (s, 3H), 2.41 (s, 3H), 1.48 (s, 12H).

Step G. 8-fluoro-2-(((2R,7aS)-2-fluorohexahydro-1H-pyrrolizin-7a-yl)methoxy)-7-(3-methoxy-2-methylnaphthalen-1-yl)-4-(2,2,2-trifluoroethoxy)pyrido[4,3-d]pyrimidine: To a solution of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (300 mg, 1.0 equiv) and 2-(3-methoxy-2-methylnaphthalen-1-yl)-4,4,5,5-tetramethyl-1,3,2-dioxaborolane (285 mg, 1.4 equiv) in methoxycyclopentane (0.6 mL) were added CataCXium A Pd G3 (50 mg, 0.1 equiv) and Cs₂CO₃ (1.5 M in H₂O, 1.37 mL, 3.0 equiv). The mixture was stirred at 80° C. for 12 hours. The mixture was diluted with water (10 mL) and extracted with ethyl acetate (2×10 mL). The organic layer was washed with brine (10 mL), dried over Na₂SO₄, concentrated and purified with reversed phase flash chromatography [C18, 0.1% formic acid condition] to afford the title compound (50 mg, 12% yield) as a yellow solid; LCMS (ESI, M+1): m/z=575.2.

Step H. (1R,5R,6R)-3-(8-fluoro-2-(((2R,7aS)-2-fluorohexahydro-1H-pyrrolizin-7a-yl)methoxy)-7-(3-methoxy-2-methylnaphthalen-1-yl)pyrido[4,3-d]pyrimidin-4-yl)-3-azabicyclo[3.2.1]octan-6-ol: To a solution of 8-fluoro-2-(((2R,7aS)-2-fluorohexahydro-1H-pyrrolizin-7a-yl)methoxy)-7-(3-methoxy-2-methylnaphthalen-1-yl)-4-(2,2,2-trifluoroethoxy)pyrido[4,3-d]pyrimidine (100 mg, 1.0 equiv) and (1R,5R,6R)-3-azabicyclo[3.2.1]octan-6-ol (44.3 mg, 2.0 equiv) in N,N-dimethylformamide (0.5 mL) were added N-ethyl-N,N-diisopropylamine (45.0 mg, 2.0 equiv) and 4 Å molecular sieves (20.0 mg). The mixture was stirred at 40° C. for 12 hours. The mixture was filtered and purified with reversed phase flash chromatography [C18, 0.1% formic acid condition] and prep-HPLC [column: Waters Xbridge 150×25 mm×5 μm; A: water (10 mM NH₄HCO₃), B: ACN, B %: 45%-75% over 8 minutes] to afford the title compound (30.0 mg, 23% yield) as a yellow solid; LCMS (ESI, M+1): m/z=602.2.

Step I. (1R,5R,6R)-3-(8-fluoro-2-(((2R,7aS)-2-fluorohexahydro-1H-pyrrolizin-7a-yl)methoxy)-7-(3-hydroxy-2-methylnaphthalen-1-yl)pyrido[4,3-d]pyrimidin-4-yl)-3-azabicyclo[3.2.1]octan-6-ol: To a solution of (1R,5R,6R)-3-(8-fluoro-2-(((2R,7aS)-2-fluorohexahydro-1H-pyrrolizin-7a-yl)methoxy)-7-(3-methoxy-2-methylnaphthalen-1-yl)pyrido[4,3-d]pyrimidin-4-yl)-3-azabicyclo[3.2.1]octan-6-ol (30.0 mg, 1.0 equiv) in dichloromethane (3 mL) was added BBr₃ (100 mg, 8.0 equiv) dropwise at 0° C. for 5 minutes. The mixture was stirred at 0° C. for 1 hour. The mixture was added into saturated aqueous NaHCO₃ (15 mL) at 0° C. The mixture was diluted with water (10 mL) and extracted with ethyl acetate (2×10 mL). The organic layer was washed with brine (10 mL), dried over Na₂SO₄, concentrated and purified with prep-HPLC [column: Waters Xbridge 150×25 mm×5 μm; A: water (10 mM NH₄HCO₃), B: ACN, B %: 37%-67% over 8 minutes] to afford the title compound (5.21 mg, 17% yield) as a white solid; ¹H NMR (400 MHZ, METHANOL-d₄) δ=9.32 (d, J=3.1 Hz, 1H), 7.68 (d, J=8.1 Hz, 1H), 7.34 (br t, J=7.4 Hz, 1H), 7.26 (s, 1H), 7.18-7.08 (m, 2H), 5.37 (br s, 1H), 5.00-4.93 (m, 2H), 4.33 (br d, J=10.5 Hz, 2H), 4.27-4.19 (m, 1H), 3.79 (br d, J=2.0 Hz, 1H), 3.49 (br dd, J=5.1, 12.7 Hz, 1H), 3.27-3.17 (m, 3H), 3.05-2.97 (m, 1H), 2.44-2.36 (m, 1H), 2.30-2.15 (m, 4H), 2.12 (s, 3H), 2.05-1.73 (m, 6H), 1.42 (br d, J=12.6 Hz, 1H); LCMS (ESI, M+1): m/z=588.2.

### Example 787

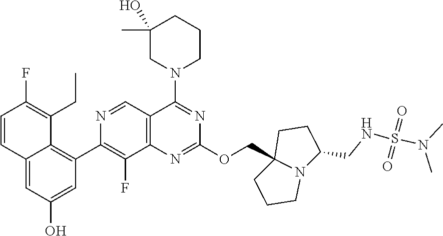 [SMILES: CCc1c(F)ccc2cc(O)cc(-c3ncc4c(N5CCC[C@@](C)(O)C5)nc(OC[C@]56CCCN5[C@@H](CNS(=O)(=O)N(C)C)CC6)nc4c3F)c12]

### (R)-1-(7-(8-ethyl-7-fluoro-3-hydroxynaphthalen-1-yl)-8-fluoro-2-(((3R,7aR)-3-(((dimethylsulfamoyl)amino)methyl)tetrahydro-1H-pyrrolizin-7a(5H)-yl)methoxy)pyrido[4,3-d]pyrimidin-4-yl)-3-methylpiperidin-3-ol

The title compound was synthesized from according to the 4-step procedure described for example 720 to produce the desired compound as a white solid (0.27 formic acid salt). ¹H NMR (400 MHZ, METHANOL-d₄) δ=9.29 (d, J=8.0 Hz, 1H), 8.47-8.40 (m, 1H), 7.73-7.64 (m, 1H), 7.34-7.22 (m, 2H), 7.13-7.03 (m, 1H), 4.75-4.53 (m, 4H), 4.35 (br t, J=11.2 Hz, 1H), 4.12-3.92 (m, 1H), 3.73-3.55 (m, 2H), 3.51-3.34 (m, 3H), 2.78 (d, J=10.0 Hz, 6H), 2.54-2.29 (m, 3H), 2.28-2.13 (m, 4H), 2.13-1.96 (m, 4H), 1.93-1.74 (m, 3H), 1.30 (d, J=9.0 Hz, 3H), 0.81 (t, J=7.2 Hz, 3H); LCMS (ESI, M+1): m/z=726.3.

### Example 788

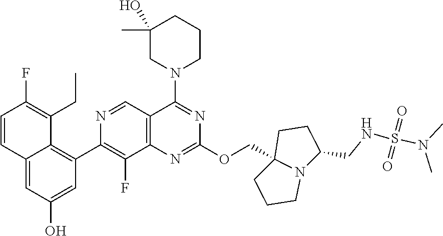 [SMILES: CCc1c(F)ccc2cc(O)cc(-c3ncc4c(N5CCC[C@@](C)(O)C5)nc(OC[C@@]56CCCN5[C@@H](CNS(=O)(=O)N(C)C)CC6)nc4c3F)c12]

### (R)-1-(7-(8-ethyl-7-fluoro-3-hydroxynaphthalen-1-yl)-8-fluoro-2-(((3R,7aS)-3-(((dimethylsulfamoyl)amino)methyl)tetrahydro-1H-pyrrolizin-7a(5H)-yl)methoxy)pyrido[4,3-d]pyrimidin-4-yl)-3-methylpiperidin-3-ol

The title compound was synthesized from according to the 4-step procedure described for example 720 to produce the desired compound as an off-white solid (0.5340 formic acid salt). ¹H NMR (400 MHZ, METHANOL-d₄) δ=9.24 (d, J=2.8 Hz, 1H), 8.52 (br s, 1H), 7.69 (dd, J=6.0, 9.0 Hz, 1H), 7.32 (d, J=2.4 Hz, 1H), 7.27 (t, J=9.2 Hz, 1H), 7.11-7.05 (m, 1H), 4.64-4.53 (m, 1H), 4.52-4.39 (m, 2H), 4.37-4.26 (m, 1H), 3.71-3.57 (m, 1H), 3.54-3.43 (m, 1H), 3.30-3.12 (m, 4H), 3.11-3.00 (m, 1H), 2.77 (d, J=1.2 Hz, 6H), 2.58-2.40 (m, 1H), 2.38-2.11 (m, 4H), 2.10-1.99 (m, 3H), 1.97-1.73 (m, 6H), 1.32 (d, J=9.0 Hz, 3H), 0.83 (q, J=7.4 Hz, 3H); LCMS (ESI, M+1): m/z=726.5.

### Example 789

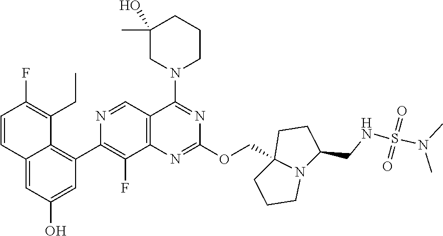 [SMILES: CCc1c(F)ccc2cc(O)cc(-c3ncc4c(N5CCC[C@@](C)(O)C5)nc(OC[C@@]56CCCN5[C@H](CNS(=O)(=O)N(C)C)CC6)nc4c3F)c12]

### (R)-1-(7-(8-ethyl-7-fluoro-3-hydroxynaphthalen-1-yl)-8-fluoro-2-(((3S,7aS)-3-(((dimethylsulfamoyl)amino)methyl)tetrahydro-1H-pyrrolizin-7a(5H)-yl)methoxy)pyrido[4,3-d]pyrimidin-4-yl)-3-methylpiperidin-3-ol

The title compound was synthesized from according to the 4-step procedure described for example 720 to produce the desired compound as a white solid (0.54 formic acid salt). ¹H NMR (400 MHZ, METHANOL-d₄) δ=9.24 (s, 1H), 8.60-8.49 (m, 1H), 7.68 (dd, J=6.0, 8.8 Hz, 1H), 7.31 (d, J=2.4 Hz, 1H), 7.25 (t, J=9.2 Hz, 1H), 7.07 (dd, J=2.4, 8.4 Hz, 1H), 4.64-4.41 (m, 3H), 4.40-4.27 (m, 1H), 3.81-3.69 (m, 1H), 3.68-3.57 (m, 1H), 3.53-3.33 (m, 4H), 3.15-2.99 (m, 1H), 2.78 (d, J=4.0 Hz, 6H), 2.55-2.40 (m, 1H), 2.36-2.27 (m, 1H), 2.26-2.04 (m, 5H), 2.02-1.74 (m, 7H), 1.30 (d, J=8.4 Hz, 3H), 0.87-0.74 (m, 3H); LCMS (ESI, M+1): m/z=726.5.

### Example 790

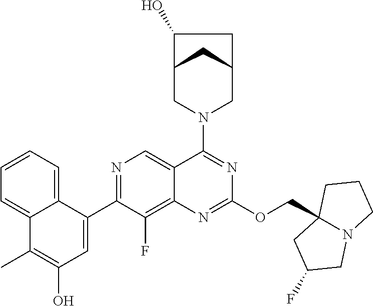 [SMILES: Cc1c(O)cc(-c2ncc3c(N4C[C@@H]5C[C@H](C4)[C@H](O)C5)nc(OC[C@@]45CCCN4C[C@H](F)C5)nc3c2F)c2ccccc12]

### (1R,5R,6R)-3-(8-fluoro-2-(((2R,7aS)-2-fluorohexahydro-1H-pyrrolizin-7a-yl)methoxy)-7-(3-hydroxy-4-methylnaphthalen-1-yl)pyrido[4,3-d]pyrimidin-4-yl)-3-azabicyclo[3.2.1]octan-6-ol

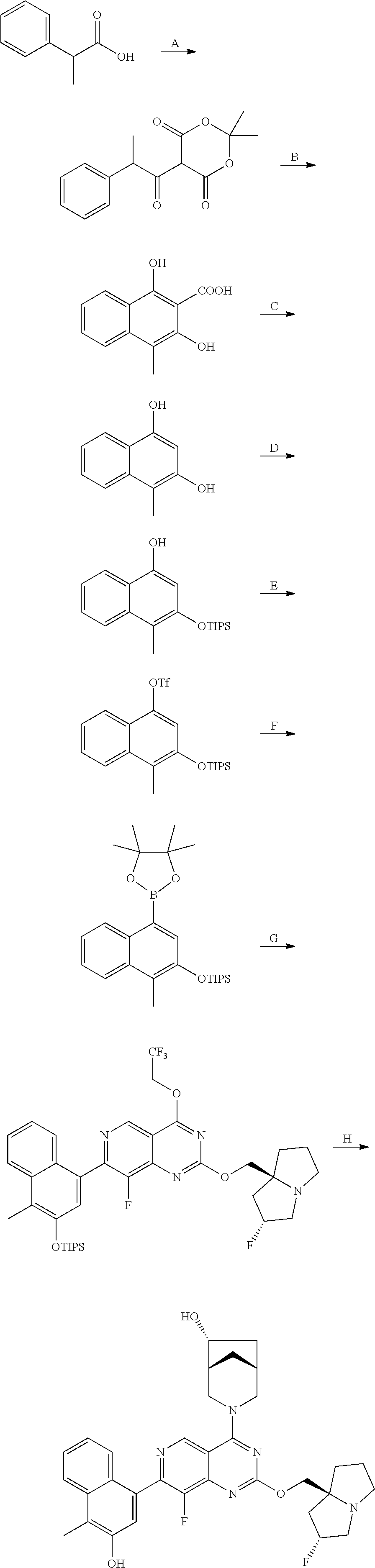

Step A. 2,2-dimethyl-5-(2-phenylpropanoyl)-1,3-dioxane-4,6-dione: To a mixture of 2-phenylpropanoic acid (10.0 g, 1.0 equiv), 2,2-dimethyl-1,3-dioxane-4,6-dione (10.5 g, 1.1 equiv), DMAP (732 mg, 0.10 equiv) and DIEA (18.5 g, 2.1 equiv) in ACN (100 mL) was added pivaloyl chloride (8.83 g, 1.1 equiv) at 20° C. The reaction was stirred at 45° C. for 2 hours. The mixture was extracted with EtOAc (3×50 mL). The combined organic layers were dried over anhydrous sodium sulfate, concentrated to afford the title compound (35.0 g, crude) as a yellow oil.

Step B. 1,3-dihydroxy-4-methyl-2-naphthoic acid: A mixture of 2,2-dimethyl-5-(2-phenylpropanoyl)-1,3-dioxane-4,6-dione (35.0 g, 1.0 equiv) in CF₃SO₃H (595 g, 31 equiv) was stirred at 10° C. for 2 hours. The mixture was quenched with H₂O (50 mL) and filtered to afford the title compound (25.0 g, crude) as a yellow solid. LCMS (ESI, M−44): m/z=174.8.

Step C. 4-methylnaphthalene-1,3-diol: A mixture of 1,3-dihydroxy-4-methyl-2-naphthoic acid (25.0 g, 1.0 equiv) in ACN (125 mL) and H₂O (125 mL) was stirred at 90° C. for 12 hours. The mixture was concentrated, diluted with water (100 mL) and extracted with EtOAc (2×200 mL). The combined organic layers were washed with brine, dried over anhydrous sodium sulfate, concentrated, and purified by prep-HPLC [column: Phenomenex luna C18 150×25 mm×10 um; mobile phase: [water (FA)-ACN]; B %: 24%-54%, 10 min] to afford the title compound (3.00 g, 13% yield) as a yellow solid. LCMS (ESI, M+1): m/z=175.1.

Step D. 4-methyl-3-((triisopropylsilyl)oxy)naphthalen-1-ol: To a mixture of 4-methylnaphthalene-1,3-diol (2.50 g, 1.0 equiv) and DIEA (14.8 g, 8.0 equiv) in DCM (25 mL) was added TIPSCl (2.49 g, 0.90 equiv). The reaction was stirred at 0° C. for 20 min. The mixture was quenched with H₂O (50 mL) and extracted with EtOAc (3×30 mL). The combined organic layers were washed with brine, dried over anhydrous sodium sulfate, concentrated and purified by prep-HPLC [column: Phenomenex Luna C18 200×40 mm×10 um; mobile phase: [water (TFA)-ACN]; B %: 70%-100%, 10 min] to afford the title compound (1.20 g, 25% yield) as a yellow oil. LCMS (ESI, M+1): m/z=331.3.

Step E. 4-methyl-3-((triisopropylsilyl)oxy)naphthalen-1-yl trifluoromethanesulfonate: To a mixture of 4-methyl-3-((triisopropylsilyl)oxy)naphthalen-1-ol (500 mg, 1.0 equiv), DIEA (1.56 g, 8.0 equiv) and 4 Å molecular sieves (100 mg) in DCM (5 mL) was added Tf₂O (85.0 mg, 2.0 equiv) at −40° C. The reaction was stirred at −40° C. for 0.5 hour. The mixture was quenched with H₂O (5 mL) and extracted with DCM (3×10 mL). The combined organic layers were washed with brine, dried over anhydrous sodium sulfate, concentrated, and purified by column chromatography (SiO₂, Petroleum ether) to afford the title compound (450 mg, 51% yield) as a yellow oil. LCMS (ESI, M+1): m/z=463.1.

Step F. triisopropyl((1-methyl-4-(4,4,5,5-tetramethyl-1,3,2-dioxaborolan-2-yl)naphthalen-2-yl)oxy) silane: To a mixture of 4-methyl-3-((triisopropylsilyl)oxy)naphthalen-1-yl trifluoromethanesulfonate (400 mg, 1.0 equiv), 4,4,5,5-tetramethyl-1,3,2-dioxaborolane (1.11 g, 10 equiv) and TEA (262 mg, 3.0 equiv) in ACN (5 mL) was added cis-dichloro-1,1′-bis(diphenylphosphino)ferrocene palladium(II) (63.2 mg, 0.10 equiv). The reaction was degassed and stirred at 80° C. for 3 hours under nitrogen atmosphere. The mixture was concentrated and purified by column chromatography (SiO₂, Petroleum ether) to afford the title compound (500 mg, 90% yield) as a yellow solid. LCMS (ESI, M+1): m/z=441.3.

Step G. 8-fluoro-2-(((2R,7aS)-2-fluorohexahydro-1H-pyrrolizin-7a-yl)methoxy)-7-(4-methyl-3-((triisopropylsilyl)oxy)naphthalen-1-yl)-4-(2,2,2-trifluoroethoxy)pyrido[4,3-d]pyrimidine: To a mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (451 mg, 1.2 equiv) and Cs₂CO₃ (835 mg, 3.0 equiv) in methoxycyclopentane (6 mL) and H₂O (2 mL) was added [2-(2-aminophenyl)phenyl]palladium bis(1-adamantyl)butylphosphane methanesulfonate (62.2 mg, 0.10 equiv). The reaction was degassed and stirred at 90° C. for 2 hours under nitrogen atmosphere. The mixture was filter and purified by reversed-phase HPLC (0.1% FA condition) to afford the title compound (270 mg, 39% yield) as a yellow solid. LCMS (ESI, M+1): m/z=717.3.

Step H. (1R,5R,6R)-3-(8-fluoro-2-(((2R,7aS)-2-fluorohexahydro-1H-pyrrolizin-7a-yl)methoxy)-7-(3-hydroxy-4-methylnaphthalen-1-yl)pyrido[4,3-d]pyrimidin-4-yl)-3-azabicyclo[3.2.1]octan-6-ol: To a solution of 8-fluoro-2-(((2R,7aS)-2-fluorohexahydro-1H-pyrrolizin-7a-yl)methoxy)-7-(4-methyl-3-((triisopropylsilyl)oxy)naphthalen-1-yl)-4-(2,2,2-trifluoroethoxy)pyrido[4,3-d]pyrimidine (100 mg, 1.0 equiv), (1S,5S,6R)-3-azabicyclo[3.2.1]octan-6-ol (35.4 mg, 2.0 equiv) and 4 Å molecular sieves (50 mg) in DMF (1 mL) was added DIEA (108 mg, 6.0 equiv). The reaction was stirred at 60° C. for 12 hours. The mixture was filtered and purified by prep-HPLC (column: Phenomenex luna C18 150×25 mm×10 μm; mobile phase: [water (FA)-ACN]; B %: 15%-45%, 9 min) to afford the title compound (62.4 mg, 75% yield, 0.5 μHCOOH salt) as a white solid. ¹H NMR (400 MHZ, METHANOL-d₄) δ=9.28 (s, 1H), 8.02 (d, J=8.4 Hz, 1H), 7.62-7.43 (m, 2H), 7.34-7.17 (m, 2H), 5.52-5.33 (m, 1H), 4.94 (br d, J=12.4 Hz, 1H), 4.80 (br d, J=12.8 Hz, 1H), 4.57-4.37 (m, 2H), 4.33 (td, J=5.2, 10.4 Hz, 1H), 3.86-3.61 (m, 2H), 3.58-3.47 (m, 3H), 3.27-3.15 (m, 1H), 2.59 (s, 3H), 2.56-2.44 (m, 1H), 2.43-2.35 (m, 2H), 2.31-2.20 (m, 3H), 2.19-2.09 (m, 2H), 2.07-1.96 (m, 1H), 1.96-1.88 (m, 1H), 1.86-1.76 (m, 1H), 1.37 (d, J=6.4 Hz, 1H); LCMS (ESI, M+1): m/z=588.2.

### Example 791

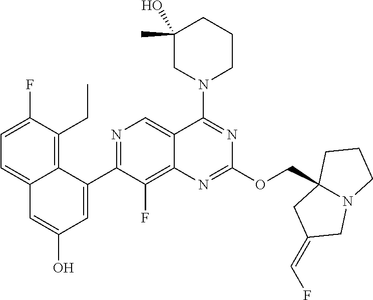 [SMILES: CCc1c(F)ccc2cc(O)cc(-c3ncc4c(N5CCC[C@@](C)(O)C5)nc(OC[C@@]56CCCN5C/C(=C\F)C6)nc4c3F)c12]

### (R)-1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol

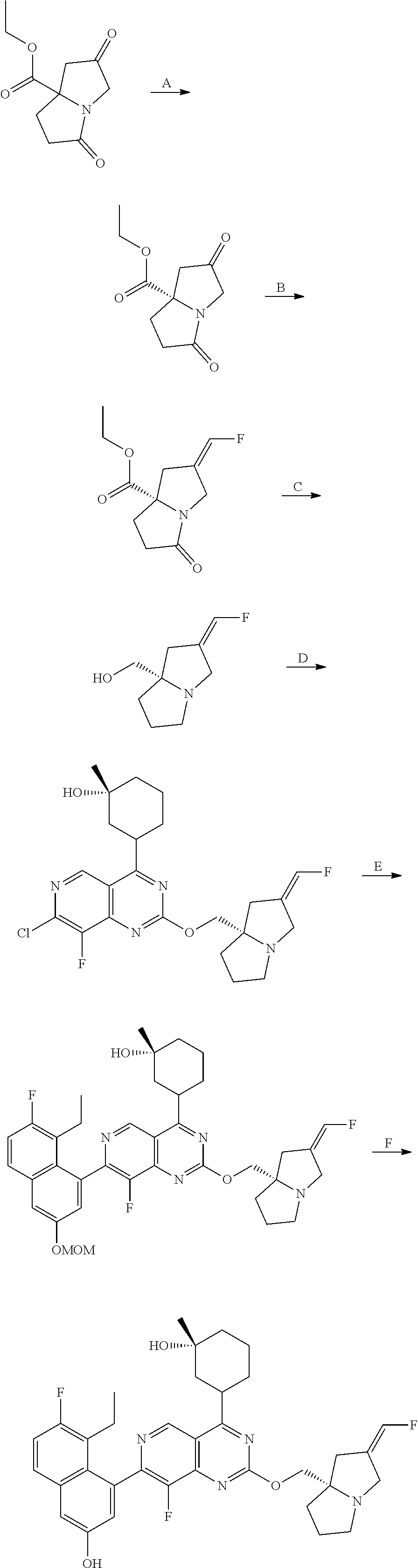

Step A: ethyl(S)-2,5-dioxotetrahydro-1H-pyrrolizine-7a(5H)-carboxylate. Racemic ethyl 2,5-dioxotetrahydro-1H-pyrrolizine-7a(5H)-carboxylate (12.0 g) was separated by SFC (Column: Chiralpak AD-3 50×4.6 mm I.D., 3 um; Mobile phase: Phase A for CO₂, and Phase B for EtOH (0.05% DEA); Gradient elution: B in A from 5% to 40%; Flow rate: 3 mL/min; Detector: DAD; Column Temp: 35C; Back Pressure: 100 Bar) to afford the tile compound (5.00 g, the first peak) as a yellow oil and the other enantiomer (6.0 g, the second peak) as a yellow oil.

Step B: ethyl (S,Z)-2-(fluoromethylene)-5-oxotetrahydro-1H-pyrrolizine-7a(5H)-carboxylate. To a solution of 2-((fluoromethyl) sulfonyl)pyridine (18.0 g, 102 mmol, 1.07 eq) in THF (800 mL) was added KHMDS (1 M, 122 mL, 1.27 eq) at −78° C., and the mixture was stirred at −78° C. for 30 min. Ethyl(S)-2,5-dioxotetrahydro-1H-pyrrolizine-7a(5H)-carboxylate (20.0 g, 96.0 mmol, 1.0 eq) in THF (100 mL) was added dropwise at −78° C., and then the reaction stirred for another 3 hour under N₂. The reaction was quenched with NH₄Cl saturated solution, acidified with HCl (3M, 102 mL) and stirred for 30 mins. The mixture was then diluted with H₂O (1.00 L), extracted with ethyl acetate (300 mL×3), dried over with Na₂SO₄, filtered and concentrated under reduced pressure to give a residue. The residue was purified by column chromatography (SiO₂, Petroleum ether/Ethyl acetate=10/1 to 3/1), and then reversed-phase HPLC.

Step C: (S,Z)-(2-(fluoromethylene)tetrahydro-1H-pyrrolizin-7a(5H)-yl)methanol. To a solution of ethyl (S,Z)-2-(fluoromethylene)-5-oxotetrahydro-1H-pyrrolizine-7a(5H)-carboxylate (2.00 g) in THF (10.0 mL) was added dropwise DIBAL-H (1 M, 96.8 mL, 10.0 eq) at 0° C. Then the reaction was stirred at this temperature for 2 hrs. The reaction was quenched with Na₂SO₄·10·H₂O (5.0 g) and filtered. The filtrate was concentrated under reduced pressure. The residue was purified by column chromatography (SiO₂, Petroleum ether/Ethyl acetate=1/0 to dichloromethane:Methanol=5/1) to give the title compound (1.20 g, 7.01 mmol, 79.6% yield) as yellow solid.

Step D: (R)-1-(7-chloro-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol. To a solution of (S,Z)-(2-(fluoromethylene)tetrahydro-1H-pyrrolizin-7a(5H)-yl)methanol (108 mg, 634 μmol, 1.05 eq) in dioxane (2.00 mL) were added 4A MS (200 mg), DIEA (351 mg, 2.72 mmol, 473 μL, 4.50 eq), and then (R)-1-(2,7-dichloro-8-fluoropyrido[4,3-d]pyrimidin-4-yl)-3-methylpiperidin-3-ol (200 mg, 603.92 μmol, 1.00 eq). The reaction was stirred at 95° C. for 2 hrs. The reaction mixture was extracted with Ethyl acetate 10.0 mL (5.00 mL×2). The combined organic layers were washed with brine (5.00 mL×2), dried over Na₂SO₄, filtered, and concentrated under reduced pressure to give a residue. The residue was purified by prep-TLC (SiO₂, Petroleum ether/Ethyl acetate=1/1, R_{f}=0.50) to give the title compound (80.0 mg, 142 μmol, 83.0% purity) as a white solid. LCMS: Rt=0.396 min, m/z=466.3, M+H⁺.

Step E: (R)-1-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol. A mixture of (R)-1-(7-chloro-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol (60.0 mg, 128 μmol, 1.00 eq), 2-(8-ethyl-7-fluoro-3-(methoxymethoxy)naphthalen-1-yl)-4,4,5,5-tetramethyl-1,3,2-dioxaborolane (69.6 mg, 193 μmol, 1.50 eq), Ad₂nBup-Pd-G3 (28.1 mg, 38.6 μmol, 0.30 eq) and K₃PO₄ (1.50 M, 258 μL, 3.00 eq) in THF (1.00 mL) was degassed and purged with N₂ for 3 times. The reaction was stirred at 60° C. under N₂ atmosphere for 2 hours. The residue was diluted with H₂O (2.00 mL) and extracted with Ethyl acetate 5.00 mL (2.50 mL×2). The combined organic layers were washed with brine 5.00 mL (2.50 mL×2), dried over Na₂SO₄, filtered, and concentrated under reduced pressure to give a residue. The residue was purified by prep-TLC (SiO₂, Petroleum ether/Ethyl acetate=1/1, R_{f}=0.70) to give the tile compound (63.0 mg, 88.5 μmol, 93.2% purity) as a white solid.

Step F: (R)-1-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol. A mixture of (R)-1-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol and HCl/dioxane (4 M, 218 μL, 10.0 eq) in MeCN (0.70 mL) was degassed and purged with N₂ for 3 times, and then the mixture was stirred at 0° C. under N₂ atmosphere for 1 hour. The residue was diluted with saturated NaHCO₃ solution (1.00 mL) and extracted with ethyl acetate 2.00 mL (1.00 mL×2). The combined organic layers were washed with brine 2.00 mL (1.00 mL×2), dried over Na₂SO₄, filtered, and concentrated under reduced pressure to give a residue. The residue was purified by prep-HPLC (column: Phenomenex Luna C18 150*25 mm*10 um; mobile phase: [water (FA)-ACN]; gradient: 15%-45% B over 10 min). Then the fractions were freeze-dried to give the title compound (17.5 mg, 25.6 μmol, FA salt, 97.4% purity) as a white solid. LCMS: Rt=0.460 min, m/z=620.3, M+H⁺.

### Example 792

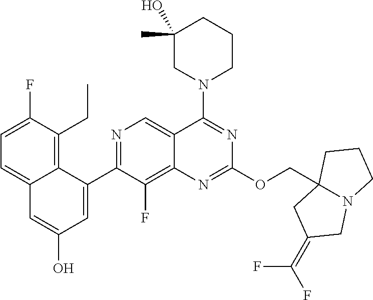 [SMILES: CCc1c(F)ccc2cc(O)cc(-c3ncc4c(N5CCC[C@@](C)(O)C5)nc(OCC56CCCN5CC(=C(F)F)C6)nc4c3F)c12]

### (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

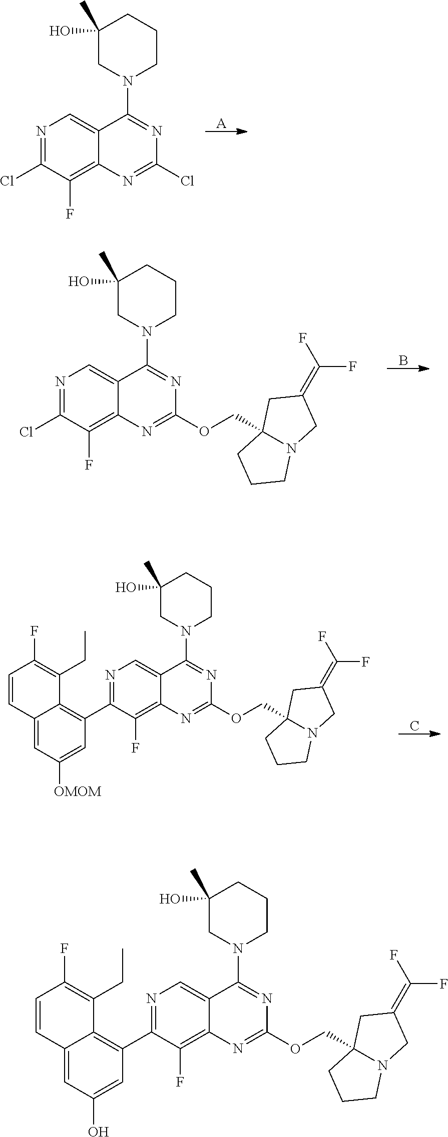

Step A: (3R)-1-(7-chloro-2-((2-(difluoromethylene)tetrahydro-1H-pyrrolizin-7a(5H)-yl)methoxy)-8-fluoropyrido[4,3-d]pyrimidin-4-yl)-3-methylpiperidin-3-ol. A mixture of (R)-1-(2,7-dichloro-8-fluoropyrido[4,3-d]pyrimidin-4-yl)-3-methylpiperidin-3-ol (200 mg, 604 μmol, 1.00 eq), (2-(difluoromethylene)tetrahydro-1H-pyrrolizin-7a(5H)-yl)methanol (114 mg, 6042 μmol, 1.00 eq) in THF (4.00 mL) was degassed and purged with N₂ for 3 times. To the mixture was added dropwise NaHMDS (1 M, 755 μL, 1.25 eq) at −10° C. The reaction was stirred at 20° C. for 3 hrs. The residue was poured into NH₄Cl (5.00 mL) and stirred for 30 min. The aqueous phase was extracted with ethyl acetate (5.00 mL×2). The combined organic phase was washed with brine (5.00 mL), dried with anhydrous Na₂SO₄, filtered, and concentrated in vacuum. The residue was purified by prep-TLC (SiO₂, petroleum ether:ethyl acetate=0:1) to give compound the title compound (130 mg, 268 μmol, 44.5% yield) as yellow solid. LCMS: RT=0.413 min, M/Z=484.3 [M+H]⁻.

Step B: (3R)-1-(2-((2-(difluoromethylene)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of (3R)-1-(7-chloro-2-((2-(difluoromethylene)tetrahydro-1H-pyrrolizin-7a(5H)-yl)methoxy)-8-fluoropyrido[4,3-d]pyrimidin-4-yl)-3-methylpiperidin-3-ol (130 mg, 268 μmol, 1.00 eq), 2-(8-ethyl-7-fluoro-3-(methoxymethoxy)naphthalen-1-yl)-4,4,5,5-tetramethyl-1,3,2-dioxaborolane (116 mg, 322 μmol, 1.20 eq), and K₃PO₄ (171 mg, 806 μmol, 3.00 eq) in dioxane (2.00 mL) H₂O (50.0 uL) was added Pd(dtbpf)Cl₂ (17.5 mg, 26.9 μmol, 0.10 eq) in one portion at 20° C. under N₂. Then the reaction was heated to 100° C. and stirred for 2 hrs. The residue was poured into ice-water (5.00 mL) and stirred for 5 min. The aqueous phase was extracted with ethyl acetate (5.00 mL×2). The combined organic phase was washed with brine (3.00 mL), dried with anhydrous Na₂SO₄, filtered, and concentrated in vacuum. The residue was purified by prep-TLC (SiO₂, petroleum ether:ethyl acetate=0:1) to give the title compound (120 mg, 176 μmol, 66% yield) as yellow solid. LCMS: RT=0.501 min, M/Z=682.6 [M+H]⁺.

Step C: (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of (3R)-1-(2-((2-(difluoromethylene)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (110 mg, 161 μmol, 1.00 eq) in MeCN (1.00 mL) was added HCl/dioxane (4 M, 0.5 mL, 12.4 eq) in one portion at 20° C. under N₂. The mixture was stirred at 20° C. for 1 hour. The mixture was concentrated in vacuum. The residue was purified by prep-HPLC (column: Welch Xtimate C18 150*25 mm*5 um; mobile phase: [water (HCl)-ACN]; gradient: 22%-52% B over 8 min) to give compound the title compound (45.6 mg, 59.3 μmol, 44.3% yield, HCl) as yellow solid. ¹H NMR: 400 MHZ CD₃OD, δ 9.63 (s, 1H), 7.77-7.74 (m, 1H), 7.42-7.41 (d, J=2.40 Hz, 1H), 7.36-7.31 (t, J=9.20 Hz, 1H), 7.21-7.20 (d, J=2.40 Hz, 1H), 5.00-4.94 (m, 2H), 4.63-4.57 (m, 1H), 4.49-4.45 (m, 1H), 4.17-4.14 (d, J=14.0 Hz, 1H), 3.74-3.70 (m, 1H), 3.51-3.50 (m, 1H), 3.41-3.35 (m, 1H), 3.17-3.14 (d, J=12.0 Hz, 1H), 2.99-2.95 (d, J=16.0 Hz, 1H), 2.51-2.19 (m, 8H), 1.92-1.90 (m, 3H), 1.40 (s, 3H), 0.93-0.88 (m, 3H). LCMS: RT=0.464 min, M/Z=638.3 [M+H]⁺.

### Example 793

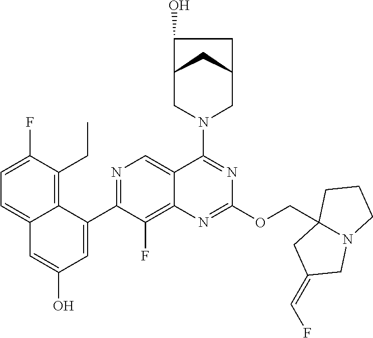 [SMILES: CCc1c(F)ccc2cc(O)cc(-c3ncc4c(N5C[C@@H]6C[C@H](C5)[C@H](O)C6)nc(OCC56CCCN5C/C(=C\F)C6)nc4c3F)c12]

### (1R,5R,6R)-3-(7-(8-ethyl-7-fluoro-3-hydroxynaphthalen-1-yl)-8-fluoro-2-(((Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol

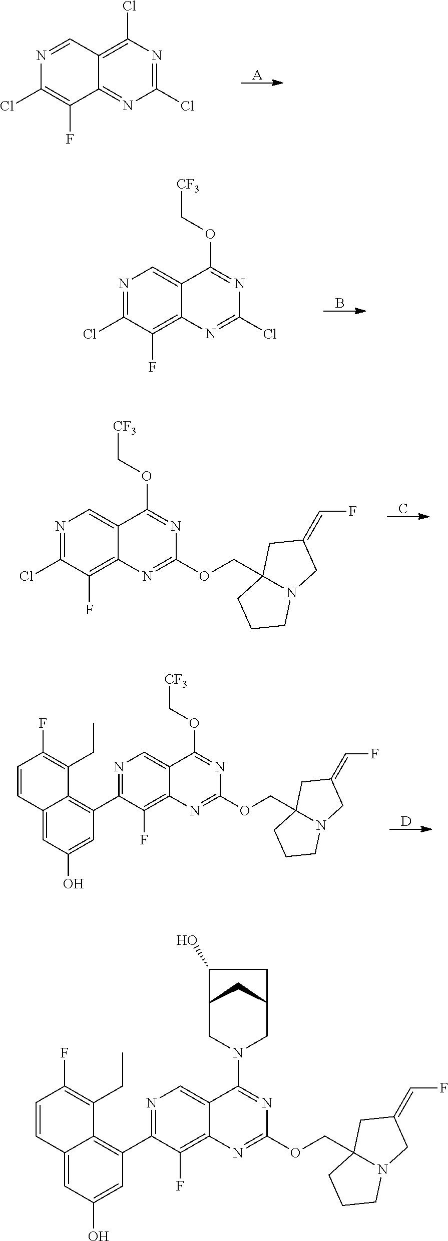

Step A. 2,7-dichloro-8-fluoro-4-(2,2,2-trifluoroethoxy)pyrido[4,3-d]pyrimidine: To a solution of 2,2,2-trifluoroethan-1-ol (872 mg, 1.1 equiv) in THF (10 mL) was added t-BuONa (2 M, 4.36 mL, 1.1 equiv). The reaction was stirred at 25° C. for 1 hour. A solution of 2,4,7-trichloro-8-fluoropyrido[4,3-d]pyrimidine (2.00 g, 1.0 equiv) in THF (15 mL) was added into the reaction mixture at −40° C. The reaction was stirred at 0° C. for 1 hour. The mixture was quenched with water (15 mL) and extracted with ethyl acetate (3×20 mL). The combined organic layers were washed with brine (20 mL), dried over anhydrous sodium sulfate, concentrated, and purified with column chromatography (SiO₂, petroleum ether/ethyl acetate=1/0 to 100/1) to afford the title compound (1.60 g, 57% yield) as white solid; ¹H NMR (400 MHZ, CHLOROFORM-d) δ=9.18 (s, 1H), 5.19-5.02 (m, 2H).

Step B. (Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (250 mg, 1.0 equiv), (Z)-(2-(fluoromethylene)tetrahydro-1H-pyrrolizin-7a(5H)-yl)methanol (203 mg, 1.5 equiv) and 4 Å molecular sieve (30.0 mg) in THF (5.0 mL) was added DIEA (307 mg, 3.0 equiv). The reaction was stirred at 40° C. for 16 hours. The mixture was diluted with water (20 mL) and extracted with ethyl acetate (3×20 mL). The combined organic layers were washed with brine, dried over anhydrous sodium sulfate, concentrated, and purified by reversed phase flash chromatography [C18, 0.1% formic acid condition] to afford the title compound (175 mg, 47% yield) as yellow solid; LCMS (ESI, M+1): m/z=451.0.

Step C. (Z)-5-ethyl-6-fluoro-4-(8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidin-7-yl)naphthalen-2-ol: To a solution of (Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine (175 mg, 1.0 equiv), 5-ethyl-6-fluoro-4-(4,4,5,5-tetramethyl-1,3,2-dioxaborolan-2-yl)naphthalen-2-ol (307 mg, 2.5 equiv) and K₃PO₄ (1.5 M, 0.776 mL, 3.0 equiv) in 1,4-dioxane (3.0 mL) was added cataCXium Pd G4 (28.3 mg, 0.1 equiv). The reaction was degassed and purged with N₂ for 3 times. The reaction was stirred at 90° C. for 1 hour. The mixture was diluted with water (20 mL) and extracted with ethyl acetate (3×20 mL). The combined organic layers were dried over anhydrous sodium sulfate, concentrated, and purified by reversed phase flash chromatography [C18, 0.1% formic acid condition] to afford the title compound (60.0 mg, 23% yield) as yellow solid; LCMS (ESI, M+1): m/z=605.2.

Step D. (1R,5R,6R)-3-(7-(8-ethyl-7-fluoro-3-hydroxynaphthalen-1-yl)-8-fluoro-2-(((Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol: To a solution of (Z)-5-ethyl-6-fluoro-4-(8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidin-7-yl)naphthalen-2-ol (60.0 mg, 1.0 equiv), (1R,5R,6R)-3-azabicyclo[3.2.1]octan-6-ol (48.7 mg, 3.0 equiv, HCl) and 4 Å molecular sieve (30 mg) in DMF (1.0 mL) was added DIEA (64.1 mg, 5.0 equiv). The reaction was stirred at 40° C. for 12 hours. The mixture was filtered and purified with prep-HPLC [Welch Xtimate C18 150×25 mm×5 um; A: water (FA), B: ACN, B %: 11%-41% over 10 min] to afford the title compound (22.0 mg 34% yield, 0.33 HCOOH) as white solid; ¹H NMR (400 MHZ, METHANOL-d₄) δ=9.29-9.16 (m, 1H), 7.72-7.61 (m, 1H), 7.34-7.19 (m, 2H), 7.06 (br d, J=16.0 Hz, 1H), 6.86-6.56 (m, 1H), 5.07-4.91 (m, 1H), 4.85-4.71 (m, 2H), 4.49-4.25 (m, 3H), 4.01 (br d, J=14.4 Hz, 1H), 3.88-3.72 (m, 1H), 3.64 (br d, J=14.4 Hz, 1H), 3.54-3.38 (m, 1H), 2.93-2.75 (m, 2H), 2.61-2.33 (m, 3H), 2.31-2.12 (m, 4H), 2.10-1.89 (m, 4H), 1.87-1.73 (m, 1H), 1.40 (br d, J=13.2 Hz, 1H), 0.87-0.71 (m, 3H); LCMS (ESI, M+1): m/z=632.3.

### Example 794

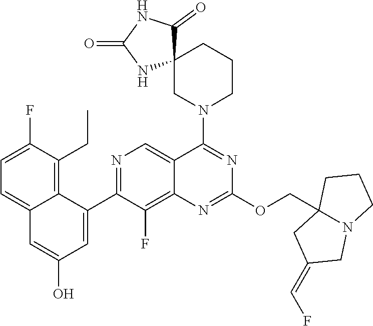 [SMILES: CCc1c(F)ccc2cc(O)cc(-c3ncc4c(N5CCC[C@]6(C5)NC(=O)NC6=O)nc(OCC56CCCN5C/C(=C\F)C6)nc4c3F)c12]

### (5R)-7-(7-(8-ethyl-7-fluoro-3-hydroxynaphthalen-1-yl)-8-fluoro-2-(((Z)-2-(fluoromethylene)tetrahydro-1H-pyrrolizin-7a(5H)-yl)methoxy)pyrido[4,3-d]pyrimidin-4-yl)-1,3,7-triazaspiro[4.5]decane-2,4-dione

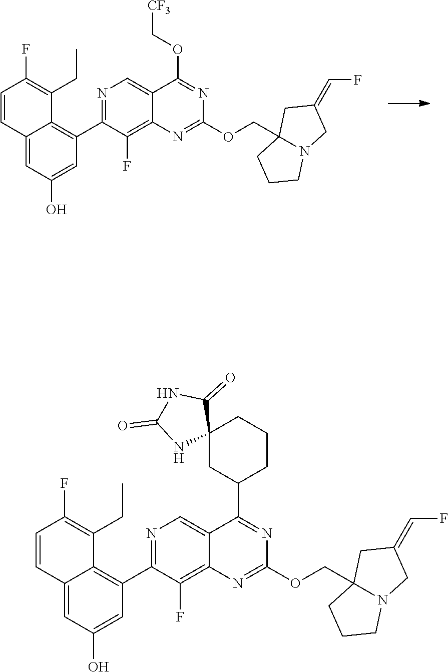 [SMILES: CCc1c(F)ccc2cc(O)cc(-c3ncc4c(OCC(F)(F)F)nc(OCC56CCCN5C/C(=C\F)C6)nc4c3F)c12 ; CCc1c(F)ccc2cc(O)cc(-c3ncc4c(C5CCC[C@]6(C5)NC(=O)NC6=O)nc(OCC56CCCN5C/C(=C\F)C6)nc4c3F)c12]

To a mixture of (Z)-5-ethyl-6-fluoro-4-(8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidin-7-yl)naphthalen-2-ol (45.0 mg, 1.0 equiv) and (R)-1,3,7-triazaspiro[4.5]decane-2,4-dione (25.2 mg, 2.0 equiv) in DMF (0.5 mL) were added DIEA (48.1 mg, 5.0 equiv) and 4 Å molecular sieve (10.0 mg). The reaction was stirred at 40° C. for 12 hours. The mixture was filtered and purified by prep-HPLC [Phenomenex luna C18 150×25 mm×10 μm; A: water (FA); B: ACN; B %: 16%-46% over 10 min] to afford the title compound (7.91 mg, 20% yield, 0.28 HCOOH) as white solid; ¹H NMR (400 MHZ, DMSO-d₆) δ=10.96-10.80 (m, 1H), 9.88 (s, 1H), 9.16-9.04 (m, 1H), 8.70 (d, J=5.2 Hz, 1H), 7.73 (s, 1H), 7.42-7.29 (m, 2H), 7.06 (s, 1H), 6.89-6.58 (m, 1H), 4.56-4.31 (m, 2H), 4.21-4.10 (m, 1H), 4.09-4.01 (m, 1H), 3.77-3.65 (m, 1H), 3.63-3.37 (m, 4H), 3.05-2.97 (m, 1H), 2.63-2.56 (m, 2H), 2.38-2.28 (m, 2H), 2.19-2.00 (m, 3H), 2.00-1.89 (m, 2H), 1.85 (br s, 2H), 1.75 (br s, 1H), 0.80-0.67 (m, 3H); LCMS (ESI, M+1): m/z=674.3.

### Example 795

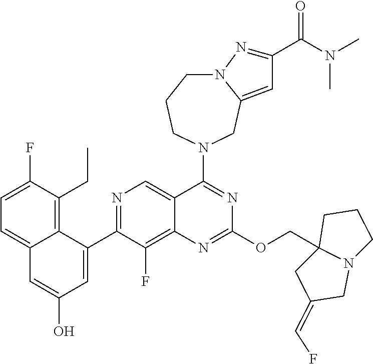 [SMILES: CCc1c(F)ccc2cc(O)cc(-c3ncc4c(N5CCCn6nc(C(=O)N(C)C)cc6C5)nc(OCC56CCCN5C/C(=C\F)C6)nc4c3F)c12]

### (Z)-5-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

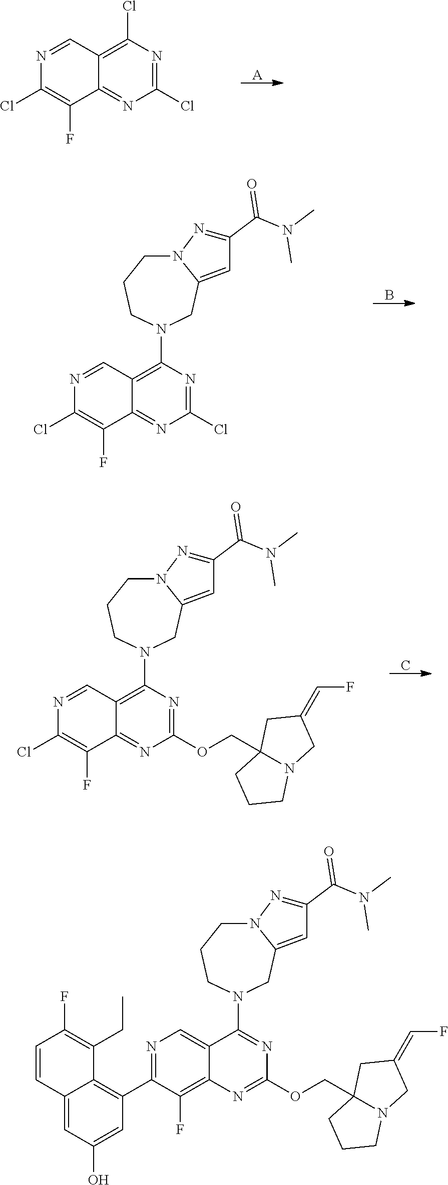

Step A. 5-(2,7-dichloro-8-fluoropyrido[4,3-d]pyrimidin-4-yl)-N,N-dimethyl-5,6,7,8-tetrahydro-4H-pyrazolo[1,5-a][1,4]diazepine-2-carboxamide: To a solution of 2,4,7-trichloro-8-fluoropyrido[4,3-d]pyrimidine (300 mg, 1.0 equiv) and DIEA (768 mg, 5.0 equiv) in DCM (10 mL) was added N,N-dimethyl-5,6,7,8-tetrahydro-4H-pyrazolo[1,5-a][1,4]diazepine-2-carboxamide (291 mg, 1.0 equiv, HCl) at −40° C. The reaction was stirred at −40° C. for 0.5 hours. The mixture was quenched with water (20.0 mL) and extracted with ethyl acetate (3×20.0 mL). The combined organic layers were dried over anhydrous sodium sulfate, filtered and concentrated to afford the title compound (450 mg, 88% yield) as yellow solid; LCMS (ESI, M+1): m/z=423.9.

Step B. (Z)-5-(7-chloro-8-fluoro-2-((2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: A solution of 5-(2,7-dichloro-8-fluoropyrido[4,3-d]pyrimidin-4-yl)-N,N-dimethyl-5,6,7,8-tetrahydro-4H-pyrazolo[1,5-a][1,4]diazepine-2-carboxamide (180 mg, 1.0 equiv) and (Z)-(2-(fluoromethylene)tetrahydro-1H-pyrrolizin-7a(5H)-yl)methanol (145 mg, 2.0 equiv) in DMSO (0.5 mL) was stirred at 80° C. for 5 hours. The mixture was diluted with water (10.0 mL) and extracted with ethyl acetate (3×20.0 mL). The combined organic layers were washed with brine, dried over anhydrous sodium sulfate, concentrated and purified with prep-TLC (SiO₂, dichloromethane/methanol=10/1) to afford the title compound (55.0 mg, 20% yield) as yellow solid; LCMS (ESI, M+1): m/z=559.3.

Step C. (Z)-5-(7-(8-ethyl-7-fluoro-3-hydroxynaphthalen-1-yl)-8-fluoro-2-((2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of (Z)-5-(7-chloro-8-fluoro-2-((2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (50.0 mg, 1.0 equiv), 5-ethyl-6-fluoro-4-(4,4,5,5-tetramethyl-1,3,2-dioxaborolan-2-yl)naphthalen-2-ol (42.4 mg, 1.5 equiv) and K₃PO₄ (1.5 M, 3.0 equiv) in 1,4-dioxane (1.0 mL) was added cataCXium Pd G4 (6.51 mg, 0.10 equiv). The reaction was degassed and purged with N₂ for 3 times. The reaction was stirred at 90° C. for 1 hour. The mixture was filtered, concentrated and purified by prep-HPLC [Welch Xtimate C18 150×25 mm×5 um; A: water (FA), B: ACN; B %: 13%-43% over 10 min] to afford the title compound (19.3 mg, 29% yield, 0.34 HCOOH) as yellow solid; ¹H NMR (400 MHZ, METHANOL-d₄) δ=9.19 (s, 1H), 7.72-7.62 (m, 1H), 7.35-7.20 (m, 2H), 7.05 (d, J=2.4 Hz, 1H), 6.81-6.55 (m, 2H), 5.37-5.29 (m, 1H), 5.27-5.18 (m, 1H), 4.56 (br d, J=5.4 Hz, 2H), 4.48-4.35 (m, 3H), 4.35-4.29 (m, 1H), 3.94 (br d, J=14.8 Hz, 1H), 3.57 (br d, J=14.8 Hz, 1H), 3.32 (br s, 3H), 3.28-3.22 (m, 1H), 3.08 (s, 3H), 2.85-2.68 (m, 2H), 2.56-2.36 (m, 4H), 2.21-2.09 (m, 2H), 2.06-1.86 (m, 3H), 0.79 (t, J=7.4 Hz, 3H); LCMS (ESI, M+1): m/z=713.6.

### Example 796

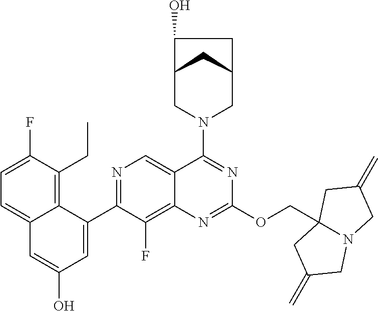 [SMILES: C=C1CN2CC(=C)CC2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(2-((2,6-dimethylenetetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol

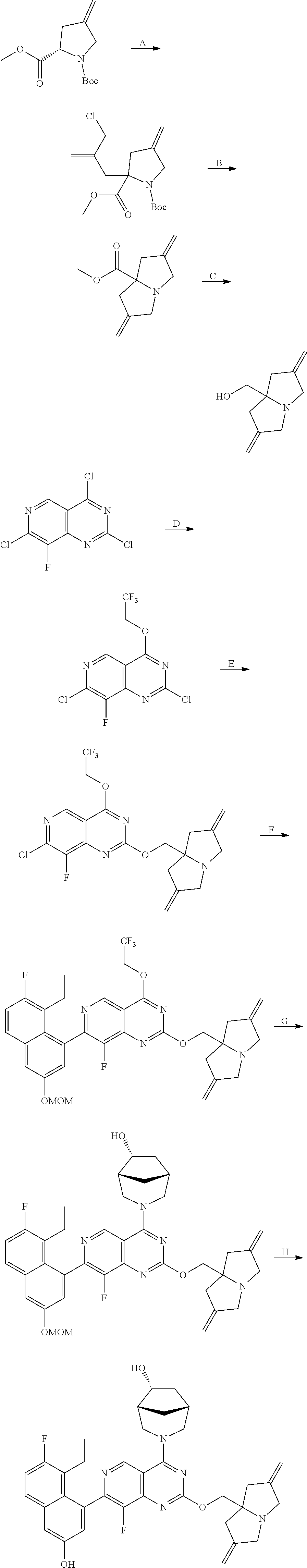

Step A. 1-(tert-butyl) 2-methyl 2-(2-(chloromethyl) allyl)-4-methylenepyrrolidine-1,2-dicarboxylate: To a solution of 1-(tert-butyl) 2-methyl(S)-4-methylenepyrrolidine-1,2-dicarboxylate (5.00 g, 1.00 equiv) in THF (50 mL) was added dropwise LiHMDS (1 M, 41.4 mL, 2.00 equiv) at −78° C. The mixture was stirred at this temperature for 1 hour, and then 3-chloro-2-(chloromethyl) prop-1-ene (6.48 g, 2.50 equiv) was added dropwise at −78° C. The reaction was stirred at 25° C. for 12 hours. The mixture was diluted with water (50 mL) and extracted with EtOAc (3×50 mL). The combined organic layers were washed with brine (100 mL), dried over Na₂SO₄, filtered and concentrated under reduced pressure. The residue was purified with column chromatography (SiO₂, Petroleum ether/Ethyl acetate=0/1 to 10/1) to afford the title compound (3.00 g, 44% yield) as a yellow oil; ¹H NMR (400 MHZ, CDCl₃) δ=5.34 (d, J=7.6 Hz, 1H), 5.07-4.94 (m, 3H), 4.28-3.96 (m, 4H), 3.75 (s, 3H), 3.38-3.21 (m, 1H), 2.91-2.68 (m, 3H), 1.47 (s, 9H); LCMS (ESI, M−1): m/z=230.1.

Step B. methyl 2,6-dimethylenetetrahydro-1H-pyrrolizine-7a(5H)-carboxylate: To a solution of 1-(tert-butyl) 2-methyl 2-(2-(chloromethyl) allyl)-4-methylenepyrrolidine-1,2-dicarboxylate (12.5 g, 1.00 equiv) in DCM (100 mL) was added TFA (64.8 g, 15.0 equiv). The reaction was stirred at 25° C. for 12 hrs. The mixture was concentrated under reduced pressure. The residue was diluted with H₂O (100 mL) and washed with MTBE (2×100 mL). The aqueous phase was adjusted to pH=9 with saturated Na₂CO₃ aqueous at 20° C. for 1 hour and extracted with ethyl acetate (3×100 mL). The combined organic phase was washed with brine (2×100 mL), dried with anhydrous Na₂SO₄, filtered and concentrated in vacuum to afford the title compound (6.00 g, 61% yield, 74% purity) as a yellow oil; ¹H NMR (400 MHZ, CDCl₃) δ=4.89 (d, J=19.2 Hz, 4H), 4.06-3.65 (m, 5H), 3.18 (d, J=16.4 Hz, 2H), 2.93-2.88 (m, 2H), 2.50 (d, J=16.8 Hz, 1H); LCMS (ESI, M−1): m/z=194.2.

Step C. (2,6-dimethylenetetrahydro-1H-pyrrolizin-7a(5H)-yl)methanol: To a solution of LAH (2.5 M, 6.99 mL, 0.75 equiv) in THF (50.0 mL) was added dropwise a solution of methyl 2,6-dimethylenetetrahydro-1H-pyrrolizine-7a(5H)-carboxylate (4.50 g, 1.00 equiv) in THF (10 mL) at 0° C.˜10° C. The reaction was stirred at 10° C. for 2 hours. The mixture was quenched with water (0.81 mL), 15% NaOH (0.81 mL) and water (2.43 mL) in turn at 0° C.˜5° C. and stirred at 25° C. for 30 mins. The mixture was filtered and concentrated in vacuum to afford the title compound (3.00 g, 72% yield) as a colorless oil; ¹H NMR (400 MHZ, CDCl₃) δ=4.96-4.84 (m, 4H), 3.67 (d, J=14.8 Hz, 2H), 3.29-3.23 (m, 4H), 3.11-2.98 (m, 1H), 2.55-2.51 (m, 2H), 2.42-2.37 (m, 2H); LCMS (ESI, M+1): m/z=166.1.

Step D. 2,7-dichloro-8-fluoro-4-(2,2,2-trifluoroethoxy)pyrido[4,3-d]pyrimidine: To a solution of CF₃CH₂OH (2.28 g, 1.15 equiv) in THF (40 mL) was added t-BuONa (2.09 g, 1.10 equiv) at 0° C. The reaction was stirred for 0.5 hours. A solution of 2,4,7-trichloro-8-fluoropyrido[4,3-d]pyrimidine (5.00 g, 1.00 equiv) in THF (25.0 mL) was added dropwise at −50° C. under N₂ atmosphere. The reaction was stirred at this temperature for 1.5 hours. The mixture was diluted with H₂O (50 mL) and extracted with ethyl acetate (3×30 mL). The combined organic layers were washed with brine (50.0 mL), dried over Na₂SO₄, filtered and concentrated under reduced pressure. The crude product was triturated with MTBE (50.0 mL) at 25° C. for 5 hours to afford the title compound (4.00 g, 64% yield) as a white solid; LCMS (ESI, M+1): m/z=315.9.

Step E. 7-chloro-2-((2,6-dimethylenetetrahydro-1H-pyrrolizin-7a(5H)-yl)methoxy)-8-fluoro-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (1.05 g, 1.10 equiv) and (2,6-dimethylenetetrahydro-1H-pyrrolizin-7a(5H)-yl)methanol (500 mg, 1.00 eq) in dioxane (5.00 mL) was added DIEA (1.56 g, 4.00 equiv). The reaction was stirred at 60° C. for 6 hours. The mixture was quenched with saturated NH₄Cl (1 mL) aqueous at 0° C. and extracted with DCM (3×2 mL). The combined organic layers were washed with brine (5 mL), dried over Na₂SO₄, filtered, concentrated under reduced pressure and purified with column chromatography (SiO₂, Petroleum ether/Ethyl acetate=50/1 to 1/1) to afford the title compound (500 mg, 35% yield) as a yellow solid; ¹H NMR (400 MHZ, CDCl₃) δ=8.92 (s, 1H), 4.98-4.94 (m, 3H), 4.91-4.28 (m, 6H), 4.30 (s, 2H), 3.74 (d, J=14.4 Hz, 2H), 3.24 (d, J=14.4 Hz, 2H), 2.69 (d, J=16.4 Hz, 2H), 2.47 (d, J=16.0 Hz, 2H); LCMS (ESI, M+1): m/z=445.0.

Step F. 2-((2,6-dimethylenetetrahydro-1H-pyrrolizin-7a(5H)-yl)methoxy)-7-(8-ethyl-7-fluoro-3-(methoxymethoxy)naphthalen-1-yl)-8-fluoro-4-(2,2,2-trifluoroethoxy)pyrido[4,3-d]pyrimidine: To a solution of 7-chloro-2-((2,6-dimethylenetetrahydro-1H-pyrrolizin-7a(5H)-yl)methoxy)-8-fluoro-4-(2,2,2-trifluoroethoxy)pyrido[4,3-d]pyrimidine (340 mg, 1.00 equiv) and 2-(8-ethyl-7-fluoro-3-(methoxymethoxy)naphthalen-1-yl)-4,4,5,5-tetramethyl-1,3,2-dioxaborolane (330 mg, 1.20 equiv) in methoxy cyclopentane (6.8 mL) were added Cs₂CO₃ aqueous (1 M, 2.29 mL, 3.00 equiv) and AdanBuP-Pd-G₃ (83.5 mg, 0.15 equiv). The reaction was stirred at 90° C. for 3 hours. The mixture was diluted with H₂O (5 mL) and extracted with MTBE (5 mL). The organic phase was washed with brine (5.00 mL), dried over Na₂SO₄, filtered, concentrated under reduced pressure and purified with prep-TLC (Petroleum ether:Ethyl acetate=1:1.5) to afford the title compound (160 mg, 30% yield) was obtained as a brown solid; LCMS (ESI, M+1): m/z=643.3.

Step G. (1R,5R,6R)-3-(2-((2,6-dimethylenetetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of 2-((2,6-dimethylenetetrahydro-1H-pyrrolizin-7a(5H)-yl)methoxy)-7-(8-ethyl-7-fluoro-3-(methoxymethoxy)naphthalen-1-yl)-8-fluoro-4-(2,2,2-trifluoroethoxy)pyrido[4,3-d]pyrimidine (140 mg, 1.00 equiv) and (1R,5R,6R)-3-azabicyclo[3.2.1]octan-6-ol (53.4 mg, 1.50 equiv, HCl salt) in DMF (0.70 mL) and MeCN (0.70 mL) was added K₂CO₃ (90.3 mg, 3.00 equiv). The reaction was stirred at 40° C. for 3 hours. The mixture was diluted with H₂O (10 mL) and extracted with ethyl acetate (10 mL). The organic phase was washed with brine (3×10 mL), dried over Na₂SO₄, filtered, concentrated under reduced pressure and purified with prep-TLC (DCM:MeOH=10:1) to afford the title compound (125 mg, 82% yield) as a yellow solid; LCMS (ESI, M+1): m/z=670.4.

Step H. (1R,5R,6R)-3-(2-((2,6-dimethylenetetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol: To a solution of (1R,5R,6R)-3-(2-((2,6-dimethylenetetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (125 mg, 1.00 equiv) in MeCN (1.25 mL) was added HCl·dioxane (4 M, 699 μL, 15.0 equiv). The reaction was stirred at 0° C. for 0.5 hours. The mixture was quenched with saturated NaHCO₃ aqueous (10 mL) at 0° C. and extracted with ethyl acetate (2×5 mL). The combined organic layers were washed with brine (2×10 mL), dried over Na₂SO₄, filtered, concentrated under reduced pressure and purified with prep-TLC (SiO₂, Petroleum ether:Ethyl acetate=0:1) to afford the title compound (40.0 mg, 31% yield) as a yellow solid; ¹H NMR (400 MHZ, MeOD) δ=9.29-9.16 (m, 1H), 7.67 (m, 1H), 7.30 (d, J=2.8 Hz, 1H), 7.24 (m, 1H), 7.06 (m, 1H), 5.02 (m, 4H), 4.82-4.71 (m, 2H), 4.37-4.28 (m, 3H), 3.84-3.73 (m, 3H), 3.52-3.34 (m, 3H), 2.81 (d, J=16.4 Hz, 2H), 2.59 (d, J=16.8 Hz, 2H), 2.54-2.44 (m, 1H), 2.40 (s, 1H), 2.28-2.09 (m, 3H), 1.92 (m, 1H), 1.84-1.75 (m, 1H), 1.45-1.33 (m, 1H), 0.80 (m, 3H); LCMS (ESI, M+1): m/z=626.2.

### Example 797

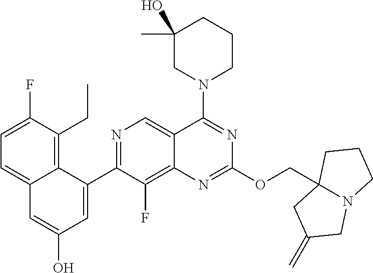 [SMILES: C=C1CN2CCCC2(COc2nc(N3CCC[C@](C)(O)C3)c3cnc(-c4cc(O)cc5ccc(F)c(CC)c45)c(F)c3n2)C1]

### (3R)-1-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-3-methylpiperidin-3-ol

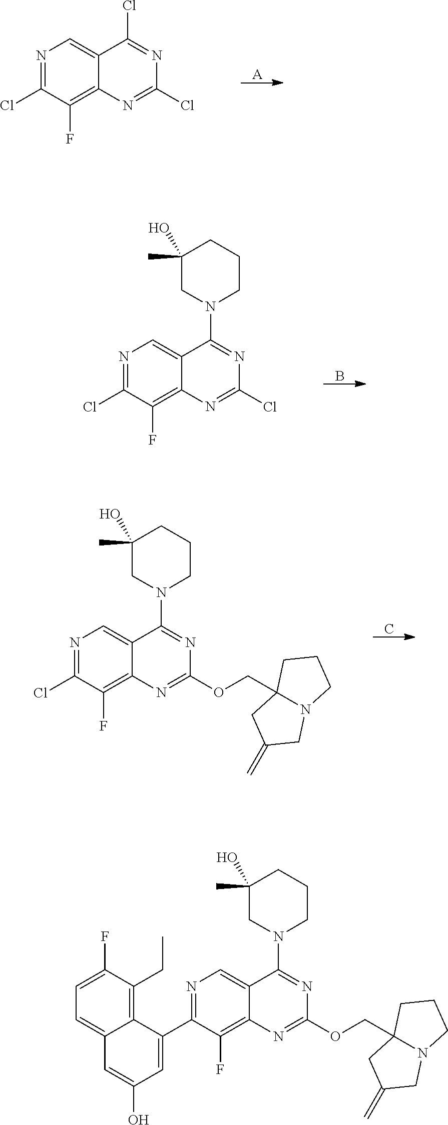

Step A. (R)-1-(2,7-dichloro-8-fluoropyrido[4,3-d]pyrimidin-4-yl)-3-methylpiperidin-3-ol: To a solution of 2,4,7-trichloro-8-fluoropyrido[4,3-d]pyrimidine (200 mg, 1.0 equiv) and DIEA (307.16 mg, 3.0 equiv) in DCM (2.0 mL) was added (R)-3-methylpiperidin-3-ol (96.1 mg, 0.8 equiv, HCl) at −40° C. The reaction was stirred at −40° C. for 1 hour. The mixture was quenched with water (5.0 mL) and extracted with ethyl acetate (3×10.0 mL). The combined organic layers were washed with brine (10.0 mL), dried over anhydrous sodium sulfate, concentrated and purified with column chromatography (SiO₂, petroleum ether/ethyl acetate=30/1 to 1/1) to afford the title compound (95.0 mg, 36% yield) as yellow solid; LCMS (ESI, M+1): m/z=331.0.

Step B. (3R)-1-(7-chloro-8-fluoro-2-((2-methylenetetrahydro-1H-pyrrolizin-7a(5H)-yl)methoxy)pyrido[4,3-d]pyrimidin-4-yl)-3-methylpiperidin-3-ol: To a solution of (R)-1-(2,7-dichloro-8-fluoropyrido[4,3-d]pyrimidin-4-yl)-3-methylpiperidin-3-ol (65.0 mg, 1.0 equiv), (2-methylenetetrahydro-1H-pyrrolizin-7a(5H)-yl)methanol (45.0 mg, 1.5 equiv) and 4 Å molecular sieve (10.0 mg) in THF (1.0 mL) was added DIEA (76.1 mg, 3.0 equiv). The reaction was stirred at 60° C. for 24 hours. The mixture was quenched with water (5.0 mL) and extracted with ethyl acetate (3×10.0 mL). The combined organic layers were washed with brine (15.0 mL), dried over anhydrous sodium sulfate, concentrated and purified by reversed phase flash chromatography [C18, 0.1% formic acid condition] to afford the title compound (50.0 mg, 57% yield) as white solid; LCMS (ESI, M+1): m/z=448.1.

Step C. (3R)-1-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-3-methylpiperidin-3-ol: To a solution of (3R)-1-(7-chloro-8-fluoro-2-((2-methylenetetrahydro-1H-pyrrolizin-7a(5H)-yl)methoxy)pyrido[4,3-d]pyrimidin-4-yl)-3-methylpiperidin-3-ol (50.0 mg, 1.0 equiv), 5-ethyl-6-fluoro-4-(4,4,5,5-tetramethyl-1,3,2-dioxaborolan-2-yl)naphthalen-2-ol (42.3 mg, 1.2 equiv) and K₃PO₄ (1.5 M, 3.0 equiv) in dioxane (1.0 mL) was added cataCXium Pd G4 (8.13 mg, 0.10 equiv). The reaction was degassed and purged with N₂ for 3 times. The reaction was stirred at 90° C. for 1 hour. The mixture was concentrated and purified with prep-HPLC [Phenomenex luna C18 150×25 mm×10 μm; A: water (FA), B: ACN, B %: 18%-48% over 10 min] to afford the title compound (4.29 mg, 5.9% yield, 0.51 HCOOH) as white solid; ¹H NMR (400 MHZ, DMSO-d₆) δ=9.73 (s, 1H), 9.34-9.08 (m, 1H), 7.76 (dd, J=6.0, 8.8 Hz, 1H), 7.42-7.27 (m, 2H), 7.03 (d, J=2.0 Hz, 1H), 4.94-4.85 (m, 2H), 4.82-4.66 (m, 1H), 4.40-4.26 (m, 1H), 4.11-3.99 (m, 3H), 3.65-3.54 (m, 2H), 3.21 (br s, 1H), 3.02-2.97 (m, 1H), 2.62-2.54 (m, 2H), 2.40-2.23 (m, 3H), 2.18-2.08 (m, 1H), 2.04-1.93 (m, 2H), 1.90-1.75 (m, 2H), 1.72-1.62 (m, 4H), 1.16 (br d, J=8.8 Hz, 3H), 0.73 (q, J=7.2 Hz, 3H); LCMS (ESI, M+1): m/z=602.3.

### Example 798

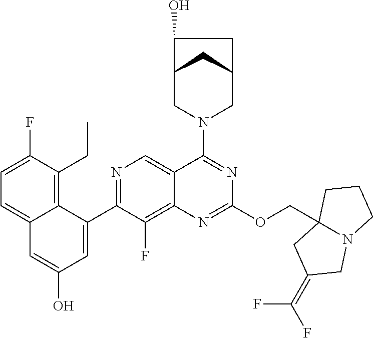 [SMILES: CCc1c(F)ccc2cc(O)cc(-c3ncc4c(N5C[C@@H]6C[C@H](C5)[C@H](O)C6)nc(OCC56CCCN5CC(=C(F)F)C6)nc4c3F)c12]

### (1R,5R,6R)-3-(2-((2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol

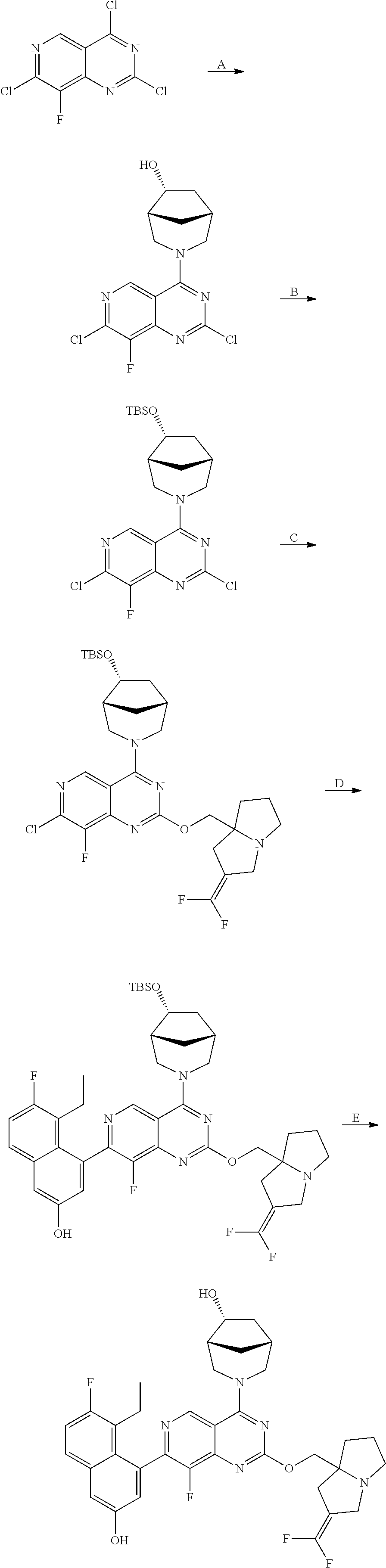

Step A. (1R,5R,6R)-3-(2,7-dichloro-8-fluoropyrido[4,3-d]pyrimidin-4-yl)-3-azabicyclo[3.2.1]octan-6-ol: To a solution of 2,4,7-trichloro-8-fluoropyrido[4,3-d]pyrimidine (2.00 g, 1.0 equiv) and DIEA (3.07 g, 3.0 equiv) in DCM (40 mL) was slowly added (1R,5R,6R)-3-azabicyclo[3.2.1]octan-6-ol (1.01 g, 1.0 equiv) at −40° C. The reaction was stirred at −40° C. for 1 hour. The mixture was quenched with water (10 mL) and extracted with dichloromethane (2× 5 mL). The combined organic layers were dried over anhydrous sodium sulfate, concentrated to afford the title compound (3.50 g, crude) as yellow solid; LCMS (ESI, M+1): m/z=343.1.

Step B. 4-((1R,5R,6R)-6-((tert-butyldimethylsilyl)oxy)-3-azabicyclo[3.2.1]octan-3-yl)-2,7-dichloro-8-fluoropyrido[4,3-d]pyrimidine: To a solution of (1R,5R,6R)-3-(2,7-dichloro-8-fluoropyrido[4,3-d]pyrimidin-4-yl)-3-azabicyclo[3.2.1]octan-6-ol (2.60 g, 1.0 equiv), tert-butylchlorodimethylsilane (2.28 g, 2.0 equiv) and DMAP (463 mg, 0.5 equiv) in DMF (30 mL) was added 1H-imidazole (1.6 g, 3.0 equiv). The reaction was stirred at 40° C. for 2 hours. The mixture was diluted with water (50 mL) and extracted with ethyl acetate (2× 50 mL). The combined organic layers were dried over anhydrous sodium sulfate, concentrated and purified with silica gel chromatography (SiO₂, Petroleum ether/Ethyl acetate=20/1 to 10/1) to afford the title compound (1.00 g, 28% yield) as white solid; LCMS (ESI, M+1): m/z=457.2.

Step C. 4-((1R,5R,6R)-6-((tert-butyldimethylsilyl)oxy)-3-azabicyclo[3.2.1]octan-3-yl)-7-chloro-2-((2-(difluoromethylene)tetrahydro-1H-pyrrolizin-7a(5H)-yl)methoxy)-8-fluoropyrido[4,3-d]pyrimidine: To a mixture of 4-((1R,5R,6R)-6-((tert-butyldimethylsilyl)oxy)-3-azabicyclo[3.2.1]octan-3-yl)-2,7-dichloro-8-fluoropyrido[4,3-d]pyrimidine (500 mg, 1.0 equiv), (2-(difluoromethylene)tetrahydro-1H-pyrrolizin-7a(5H)-yl)methanol (310 mg, 1.5 equiv) and DABCO (123 mg, 1.0 equiv) in THF (5 mL) and DMF (5 mL) was added Cs₂CO₃ (1.07 g, 3.0 equiv). The reaction was stirred at 30° C. for 12 hours. The mixture was diluted with water (10 mL) and extracted with ethyl acetate (2×10 mL). The organic layers were dried over Na₂SO₄, concentrated and purified with reversed phase flash chromatography [C18, 0.1% formic acid condition] to afford the title compound (130 mg, 17% yield) as yellow solid; LCMS (ESI, M+1): m/z=610.3.

Step D. 4-(4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-8-fluoropyrido[4,3-d]pyrimidin-7-yl)-5-ethyl-6-fluoronaphthalen-2-ol: To a solution of 4-((1R,5R,6R)-6-((tert-butyldimethylsilyl)oxy)-3-azabicyclo[3.2.1]octan-3-yl)-7-chloro-2-((2-(difluoromethylene)tetrahydro-1H-pyrrolizin-7a(5H)-yl)methoxy)-8-fluoropyrido[4,3-d]pyrimidine (110 mg, 1.0 equiv), K₃PO₄ (1.5 M, 318 mg, 8.3 equiv) and 5-ethyl-6-fluoro-4-(4,4,5,5-tetramethyl-1,3,2-dioxaborolan-2-yl)naphthalen-2-ol (171 mg, 3.0 equiv) in methoxycyclopentane (1 mL) was added cataCXium-A-Pd-G3 (39.0 mg, 0.3 equiv). The reaction was stirred at 90° C. for 1 hour under N₂ atmosphere. The mixture was diluted with water (20 mL) and extracted with ethyl acetate (2×10 mL). The organic layers were dried over Na₂SO₄, concentrated and purified with reversed phase flash chromatography [C18, 0.1% formic acid condition] to afford the title compound (100 mg, 59% yield) as yellow solid; LCMS (ESI, M+1): m/z=764.4.

Step E. (1R,5R,6R)-3-(2-((2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol: A solution of 4-(4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-8-fluoropyrido[4,3-d]pyrimidin-7-yl)-5-ethyl-6-fluoronaphthalen-2-ol (45 mg, 1.0 equiv) in HCl·MeOH (2 mL) was stirred at 25° C. for 0.5 hours.

The mixture was diluted with water (20 mL), adjusted to pH=7 with saturated NaHCO₃ (10 mL) and extracted with ethyl acetate (2×10 mL). The organic layers were dried over Na₂SO₄, concentrated and purified with prep-HPLC [Welch Xtimate 150×25 mm×5 μm; A: water (0.1% FA); B: ACN; B %: 15%-45% over 10 min] to afford the title compound (22.1 mg, 53% yield) as white solid; ¹H NMR (400 MHZ, METHANOL-d4) δ=9.32-9.17 (d, J=10.8 Hz, 1H), 7.67 (dd, J=6.0, 9.2 Hz, 1H), 7.30 (d, J=2.4 Hz, 1H), 7.28-7.21 (m, 1H), 7.06 (dd, J=2.4, 15.6 Hz, 1H), 5.01 (br d, J=12.4 Hz, 1H), 4.79 (br t, J=11.2 Hz, 1H), 4.54-4.44 (m, 1H), 4.42-4.28 (m, 2H), 4.01 (br d, J=14.8 Hz, 1H), 3.88-3.72 (m, 1H), 3.64 (br d, J=14 Hz, 1H), 3.48 (br dd, J=14, 18.8 Hz, 1H), 3.40-3.33 (m, 1H), 3.01-2.83 (m, 2H), 2.63 (br d, J=16.4 Hz, 1H), 2.56-2.36 (m, 2H), 2.31-1.88 (m, 8H), 1.86-1.73 (m, 1H), 1.46-1.33 (m, 1H), 0.80 (q, J=7.2 Hz, 3H); LCMS (ESI, M+1): m/z=650.3.

### Example 799

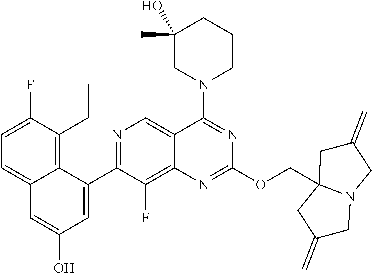 [SMILES: C=C1CN2CC(=C)CC2(COc2nc(N3CCC[C@@](C)(O)C3)c3cnc(-c4cc(O)cc5ccc(F)c(CC)c45)c(F)c3n2)C1]

### (R)-1-(2-((2,6-dimethylenetetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

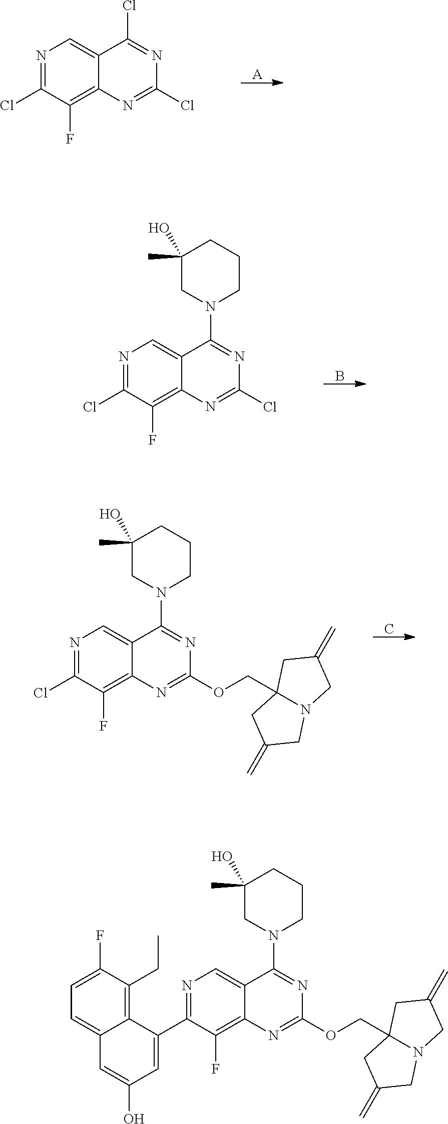

Step A. (R)-1-(2,7-dichloro-8-fluoropyrido[4,3-d]pyrimidin-4-yl)-3-methylpiperidin-3-ol: To a solution of 2,4,7-trichloro-8-fluoropyrido[4,3-d]pyrimidine (1.00 g, 1.0 equiv) and DIEA (1.54 g, 3.0 equiv) in DCM (30 mL) was added (R)-3-methylpiperidin-3-ol (456 mg, 1.0 equiv) at −40° C. The reaction was stirred at −40° C. for 2 hours. The mixture was quenched with water (1.0 mL) at 0° C. and extracted with DCM (3×50 mL). The combined organic layers were washed with brine (50.0 mL), dried over anhydrous sodium sulfate, concentrated and purified by reversed phase flash chromatography [C18, 0.1% formic acid condition] to afford the title compound (1.02 g, 77% yield) as yellow solid; ¹H NMR (400 MHZ, CHLOROFORM-d) δ=9.07 (s, 1H), 4.61-4.40 (m, 2H), 3.43-3.31 (m, 2H), 2.18-2.03 (m, 1H), 1.94-1.87 (m, 1H), 1.82-1.68 (m, 2H), 1.36 (s, 3H); LCMS (ESI, M+1, M+2): m/z=331.2, 333.2.

Step B. (R)-1-(7-chloro-2-((2,6-dimethylenetetrahydro-1H-pyrrolizin-7a(5H)-yl)methoxy)-8-fluoropyrido[4,3-d]pyrimidin-4-yl)-3-methylpiperidin-3-ol: A mixture of (R)-1-(2,7-dichloro-8-fluoropyrido[4,3-d]pyrimidin-4-yl)-3-methylpiperidin-3-ol (120 mg, 1.0 equiv), (2,6-dimethylenetetrahydro-1H-pyrrolizin-7a(5H)-yl)methanol (89.8 mg, 1.5 equiv) and DIEA (140 mg, 3 equiv) in DMF (0.05 mL) was stirred at 80° C. for 17 hours. The mixture was concentrated and purified by reversed phase flash chromatography [C18, 0.1% formic acid condition] to afford the title compound (100 mg, 49% yield) as yellow solid; LCMS (ESI, M+1): m/z=460.2.

Step C. (R)-1-(2-((2,6-dimethylenetetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of (R)-1-(7-chloro-2-((2,6-dimethylenetetrahydro-1H-pyrrolizin-7a(5H)-yl)methoxy)-8-fluoropyrido[4,3-d]pyrimidin-4-yl)-3-methylpiperidin-3-ol (50.0 mg, 1.0 equiv), 5-ethyl-6-fluoro-4-(4,4,5,5-tetramethyl-1,3,2-dioxaborolan-2-yl)naphthalen-2-ol (48.1 mg, 1.4 equiv) and Cs₂CO₃ (1.5 M, 3.0 equiv) in methoxycyclopentane (2.2 mL) was added cataCXium Pd G4 (7.92 mg, 0.1 equiv). The reaction was degassed and purged with nitrogen for 3 times. The reaction was stirred at 90° C. for 2.5 hours. The mixture was diluted with water (5 mL) and extracted with EtOAc (3×5.0 mL). The combined organic layers were dried over anhydrous sodium sulfate, concentrated and purified with prep-HPLC [Phenomenex luna C18 150×25 mm×10 μm; A: water (FA), B: ACN, B %: 19%-49% over 10 min] to afford the title compound (22.2 mg, 30% yield, 0.74 HCOOH) as yellow solid; ¹H NMR (400 MHZ, METHANOL-d₄) δ=9.24 (d, J=2.0 Hz, 1H), 7.68 (dd, J=6.0, 9.2 Hz, 1H), 7.31 (d, J=2.8 Hz, 1H), 7.25 (t, J=9.6 Hz, 1H), 7.06 (t, J=2.4 Hz, 1H), 5.18-5.10 (m, 4H), 4.57 (br d, J=12.4 Hz, 1H), 4.53 (s, 2H), 4.32 (br t, J=12.8 Hz, 1H), 4.07 (br d, J=14.8 Hz, 2H), 3.67-3.56 (m, 3H), 3.49-3.40 (m, 1H), 2.93 (br d, J=17.2 Hz, 2H), 2.71 (br d, J=16.4 Hz, 2H), 2.46 (m, 1H), 2.25-2.11 (m, 2H), 1.87-1.75 (m, 3H), 1.29 (d, J=9.2 Hz, 3H), 0.81 (q, J=7.6 Hz, 3H); LCMS (ESI, M+1): m/z=614.4.

### Example 800

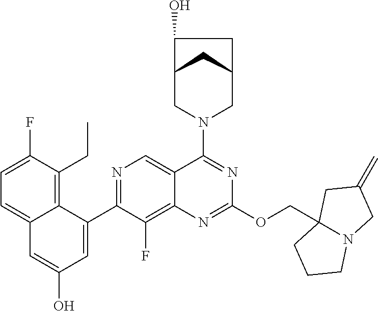 [SMILES: C=C1CN2CCCC2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-3-azabicyclo[3.2.1]octan-6-ol

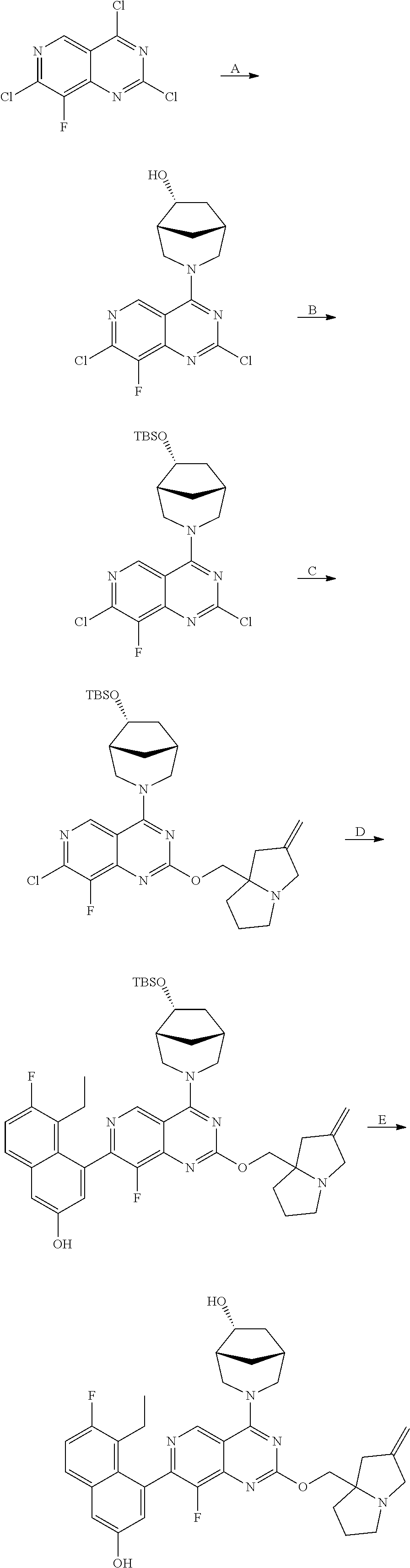

Step A. (1R,5R,6R)-3-(2,7-dichloro-8-fluoropyrido[4,3-d]pyrimidin-4-yl)-3-azabicyclo[3.2.1]octan-6-ol: To a solution of 2,4,7-trichloro-8-fluoropyrido[4,3-d]pyrimidine (200 mg, 1.0 equiv) and DIEA (307 mg, 3.0 equiv) in DCM (2.0 mL) was added (1R,5R,6R)-3-azabicyclo[3.2.1]octan-6-ol (104 mg, 0.8 equiv) at −40° C. The reaction was stirred at −40° C. for 1 hour. The mixture was quenched with water (5.0 mL) and extracted with DCM (3×10 mL). The combined organic layers were dried over anhydrous sodium sulfate, concentrated and triturated with acetonitrile at 25° C. to afford the title compound (170 mg, 63% yield) as brown oil; LCMS (ESI, M+1, M+3): m/z=343.0, 345.0.

Step B. 4-((1R,5R,6R)-6-((tert-butyldimethylsilyl)oxy)-3-azabicyclo[3.2.1]octan-3-yl)-2,7-dichloro-8-fluoropyrido[4,3-d]pyrimidine: To a solution of (1R,5R,6R)-3-(2,7-dichloro-8-fluoropyrido[4,3-d]pyrimidin-4-yl)-3-azabicyclo[3.2.1]octan-6-ol (170 mg, 1.0 equiv), DMAP (30.3 mg, 0.5 equiv) and imidazole (101 mg, 3.0 equiv) in DMF (2.0 mL) was added TBSCl (224 mg, 3.0 equiv). The reaction was stirred at 25° C. for 16 hours. The mixture was quenched with water (10 mL) and extracted with ethyl acetate (3×15 mL). The combined organic layers were washed with brine (10 mL), dried over anhydrous sodium sulfate, concentrated and purified with prep-TLC (SiO₂, petroleum ether/ethyl acetate=5/1) to afford the title compound (196 mg, 86% yield) as off-white solid; LCMS (ESI, M+1): m/z=457.1.

Step C. 4-((1R,5R,6R)-6-((tert-butyldimethylsilyl)oxy)-3-azabicyclo[3.2.1]octan-3-yl)-7-chloro-8-fluoro-2-((2-methylenetetrahydro-1H-pyrrolizin-7a(5H)-yl)methoxy)pyrido[4,3-d]pyrimidine: To a mixture of 4-((1R,5R,6R)-6-((tert-butyldimethylsilyl)oxy)-3-azabicyclo[3.2.1]octan-3-yl)-2,7-dichloro-8-fluoropyrido[4,3-d]pyrimidine (170 mg, 1.0 equiv), (2-methylenetetrahydro-1H-pyrrolizin-7a(5H)-yl)methanol (85.4 mg, 1.5 equiv) and 4 Å molecular sieve (10 mg) in THF (1.0 mL) was added DIEA (192 mg, 4.0 equiv). The reaction was stirred at 60° C. for 16 hours. The mixture was diluted with water (15 mL) and extracted with ethyl acetate (3×10 mL). The combined organic layers were washed with brine (10 mL), dried over anhydrous sodium sulfate, concentrated and purified with reversed phase flash chromatography [C18, 0.1% formic acid condition] to afford the title compound (117 mg, 55% yield) as off-white oil; LCMS (ESI, M+1): m/z=574.3.

Step D. 4-(4-((1R,5R,6R)-6-((tert-butyldimethylsilyl)oxy)-3-azabicyclo[3.2.1]octan-3-yl)-8-fluoro-2-((2-methylenetetrahydro-1H-pyrrolizin-7a(5H)-yl)methoxy)pyrido[4,3-d]pyrimidin-7-yl)-5-ethyl-6-fluoronaphthalen-2-ol: To a mixture of 4-((1R,5R,6R)-6-((tert-butyldimethylsilyl)oxy)-3-azabicyclo[3.2.1]octan-3-yl)-7-chloro-8-fluoro-2-((2-methylenetetrahydro-1H-pyrrolizin-7a(5H)-yl)methoxy)pyrido[4,3-d]pyrimidine (112 mg, 1.0 equiv), 5-ethyl-6-fluoro-4-(4,4,5,5-tetramethyl-1,3,2-dioxaborolan-2-yl)naphthalen-2-ol (92.5 mg, 1.5 equiv) and K₃PO₄ (1.5 M, 3.0 equiv) in dioxane (1.0 mL) was added cataCXium Pd G4 (14.2 mg, 0.1 equiv). The reaction was degassed and purged with N₂ for 3 times. The reaction was stirred at 90° C. for 1 hour. The mixture was diluted with water (10.0 mL) and extracted with ethyl acetate (3×10 mL). The combined organic layers were washed with brine (10.0 mL), dried over anhydrous sodium sulfate, concentrated and purified by reversed phase flash chromatography [C18, 0.1% formic acid condition] to afford the title compound (45.0 mg, 36% yield) as yellow solid; LCMS (ESI, M+1): m/z=728.6.

Step E. (1R,5R,6R)-3-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-3-azabicyclo[3.2.1]octan-6-ol: To a solution of 4-(4-((1R,5R,6R)-6-((tert-butyldimethylsilyl)oxy)-3-azabicyclo[3.2.1]octan-3-yl)-8-fluoro-2-((2-methylenetetrahydro-1H-pyrrolizin-7a(5H)-yl)methoxy)pyrido[4,3-d]pyrimidin-7-yl)-5-ethyl-6-fluoronaphthalen-2-ol (40.0 mg, 1.0 equiv) in MeOH (1.0 mL) was added HCl·MeOH (4 M, 1.0 mL). The reaction was stirred at 25° C. for 1 hour. The mixture was concentrated and purified with prep-HPLC [Phenomenex luna C18 150×25 mm×10 μm; A: water (FA), B: ACN, B %: 20%-50% over 8 min] to afford the title compound (14.4 mg, 41% yield, 0.40 HCOOH) as white solid: ¹H NMR (400 MHZ, DMSO-d₆) δ=10.31-9.59 (m, 1H), 9.39-9.21 (m, 1H), 7.76 (br dd, J=6.4, 8.4 Hz, 1H), 7.39-7.29 (m, 2H), 7.03 (dd, J=2.0, 14.8 Hz, 1H), 4.91 (br s, 2H), 4.86-4.77 (m, 1H), 4.75-4.66 (m, 1H), 4.58 (br t, J=10.4 Hz, 1H), 4.20-4.12 (m, 1H), 4.09-3.97 (m, 2H), 3.78-3.68 (m, 1H), 3.57 (br d, J=14.0 Hz, 1H), 3.21 (br d, J=14.4 Hz, 2H), 3.04-2.99 (m, 1H), 2.63-2.56 (m, 2H), 2.37 (br d, J=16.0 Hz, 3H), 2.17-2.05 (m, 3H), 2.01-1.95 (m, 1H), 1.89-1.75 (m, 3H), 1.71-1.62 (m, 2H), 1.25 (br d, J=11.6 Hz, 1H), 0.77-0.67 (m, 3H); LCMS (ESI, M+1): m/z=614.3.

### Example 801

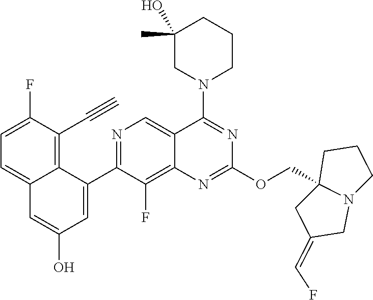 [SMILES: C#Cc1c(F)ccc2cc(O)cc(-c3ncc4c(N5CCC[C@@](C)(O)C5)nc(OC[C@]56CCCN5C/C(=C\F)C6)nc4c3F)c12]

### (3R)-1-(7-(8-ethynyl-7-fluoro-3-hydroxynaphthalen-1-yl)-8-fluoro-2-(((Z)-2-(fluoromethylene)tetrahydro-1H-pyrrolizin-7a(5H)-yl)methoxy)pyrido[4,3-d]pyrimidin-4-yl)-3-methylpiperidin-3-ol

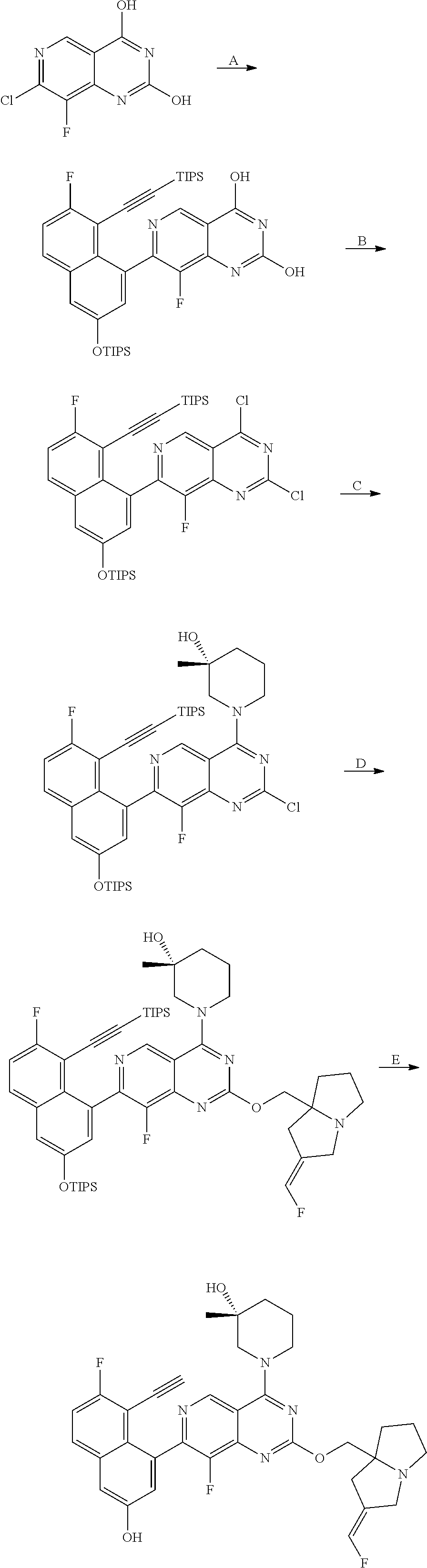

Step A. 8-fluoro-7-(7-fluoro-8-((triisopropylsilyl)ethynyl)-3-(triisopropylsilyl)oxy)naphthalen-1-yl)pyrido[4,3-d]pyrimidine-2,4-diol: To a mixture of 7-chloro-8-fluoropyrido[4,3-d]pyrimidine-2,4-diol (517 mg, 1.0 equiv), ((6-fluoro-4-(4,4,5,5-tetramethyl-1,3,2-dioxaborolan-2-yl)-5-((triisopropylsilyl)ethynyl)naphthalen-2-yl)oxy)triisopropylsilane (1.50 g, 1.0 equiv) and K₃PO₄ (1.5 M in water, 4.80 mL, 3.0 equiv) in EtOH (15 mL) was added CataCXium A Pd G3 (175 mg, 0.1 equiv). The reaction was degassed and purged with N₂ for 3 times and stirred at 80° C. for 4 hours under N₂ atmosphere. The mixture was diluted with water (50 mL) and extracted with ethyl acetate (3×50 mL). The combined organic layers were washed with brine (30 mL), dried over anhydrous sodium sulfate, concentrated and purified with column chromatography (SiO₂, Petroleum ether/Ethyl acetate=1/0 to 8/1) to afford the title compound (1.46 g, 42% yield) as yellow solid; LCMS (ESI, M+1): m/z=678.3.

Step B. 2,4-dichloro-8-fluoro-7-(7-fluoro-8-((triisopropylsilyl)ethynyl)-3-((triisopropylsilyl)oxy)naphthalen-1-yl)pyrido[4,3-d]pyrimidine: To a solution of 8-fluoro-7-(7-fluoro-8-((triisopropylsilyl)ethynyl)-3-((triisopropylsilyl)oxy)naphthalen-1-yl)pyrido[4,3-d]pyrimidine-2,4-diol (1.36 g, 1.0 equiv) and DIEA (778 mg, 3.0 equiv) in toluene (15 mL) was added POCl₃ (1.54 g, 5.0 equiv) dropwise at 0° C. The reaction was stirred at 100° C. for 1 hour. The mixture was concentrated, diluted with ethyl acetate (10 mL), slowly poured into saturated NaHCO₃ solution (20 mL) at 0° C., and extracted with ethyl acetate (2×10 mL). The combined organic layers were dried over Na₂SO₄, filtered, concentrated and purified with column chromatography (SiO₂, Petroleum ether/Ethyl acetate=1/0 to 3/1) to afford the title compound (430 mg, 30% yield) as yellow solid.

Step C. (R)-1-(2-chloro-8-fluoro-7-(7-fluoro-8-((triisopropylsilyl)ethynyl)-3-((triisopropylsilyl)oxy)naphthalen-1-yl)pyrido[4,3-d]pyrimidin-4-yl)-3-methylpiperidin-3-ol: To a solution of 2,4-dichloro-8-fluoro-7-(7-fluoro-8-((triisopropylsilyl)ethynyl)-3-((triisopropylsilyl)oxy)naphthalen-1-yl)pyrido[4,3-d]pyrimidine (430 mg, 1.0 equiv) and (R)-3-methylpiperidin-3-ol hydrochloride (95.8 mg, 1.0 equiv) in DCM (15 mL) was added DIEA (233 mg, 3.0 equiv) slowly. The reaction was stirred at −40° C. for 1 hour. The mixture was diluted with water (10 mL) and extracted with DCM (2×10 mL). The combined organic layers were dried over Na₂SO₄, filtered, concentrated and purified with silica gel column chromatography (SiO₂, Petroleum ether/Ethyl acetate=1/0 to 3/1) to afford the title compound (443 mg, 93% yield) as yellow oil; ¹H NMR (400 MHZ, CHLOROFORM-d) δ=9.41-9.23 (m, 1H), 7.74 (dd, J=5.6, 9.0 Hz, 1H), 7.35-7.28 (m, 2H), 7.21 (dd, J=2.5, 7.0 Hz, 1H), 4.69-4.33 (m, 2H), 3.58-3.25 (m, 2H), 2.27-2.08 (m, 1H), 1.98-1.88 (m, 1H), 1.86-1.62 (m, 2H), 1.38 (d, J=1.6 Hz, 3H), 1.35-1.29 (m, 3H), 1.15 (br s, 36H), 0.69-0.45 (m, 3H).

Step D (3R)-1-(8-fluoro-7-(7-fluoro-8-((triisopropylsilyl)ethynyl)-3-((triisopropylsilyl)oxy)naphthalen-1-yl)-2-(((Z)-2-(fluoromethylene)tetrahydro-1H-pyrrolizin-7a(5H)-yl)methoxy)pyrido[4,3-d]pyrimidin-4-yl)-3-methylpiperidin-3-ol: To a solution of (Z)-(2-(fluoromethylene)tetrahydro-1H-pyrrolizin-7a(5H)-yl)methanol (47.5 mg, 1.1 equiv) in THF (2 mL) was added NaH (18.1 mg, 60% purity, 1.8 equiv) at 0° C. The reaction was stirred at 25° C. for 0.5 hours. (R)-1-(2-chloro-8-fluoro-7-(7-fluoro-8-((triisopropylsilyl)ethynyl)-3-((triisopropylsilyl)oxy)naphthalen-1-yl)pyrido[4,3-d]pyrimidin-4-yl)-3-methylpiperidin-3-ol (200 mg, 1.0 equiv) was added. The reaction was stirred at 25° C. for 0.5 hours. The mixture was quenched by addition of water (10 mL), extracted with ethyl acetate (3×10 mL), dried over Na₂SO₄ and concentrated to afford the title compound (207 mg, crude) as yellow oil.

Step E. (3R)-1-(7-(8-ethynyl-7-fluoro-3-hydroxynaphthalen-1-yl)-8-fluoro-2-(((Z)-2-(fluoromethylene)tetrahydro-1H-pyrrolizin-7a(5H)-yl)methoxy)pyrido[4,3-d]pyrimidin-4-yl)-3-methylpiperidin-3-ol: To a solution of (3R)-1-(8-fluoro-7-(7-fluoro-8-((triisopropylsilyl)ethynyl)-3-((triisopropylsilyl)oxy)naphthalen-1-yl)-2-(((Z)-2-(fluoromethylene)tetrahydro-1H-pyrrolizin-7a(5H)-yl)methoxy)pyrido[4,3-d]pyrimidin-4-yl)-3-methylpiperidin-3-ol (207 mg, 1.0 equiv) in DMF (2 mL) was added CsF (339 mg, 10 equiv). The reaction was stirred at 40° C. for 1 hour. The mixture was diluted with water (10 mL), extracted with ethyl acetate (2×10 mL), concentrated and purified with prep-HPLC [column: Phenomenex luna C18 150×25 mm×10 μm; A: water (FA), B: ACN, B %: 18%-48% B over 9 min] to afford the title compound (31.3 mg, 22% yield) as yellow solid; ¹H NMR (400 MHZ, DMSO-d6) δ=9.26-9.03 (m, 1H), 7.98 (dd, J=6.0, 9.2 Hz, 1H), 7.46 (t, J=9.2 Hz, 1H), 7.39 (s, 1H), 7.21 (dd, J=2.4, 18.0 Hz, 1H), 6.91-6.61 (m, 1H), 4.93-4.62 (m, 1H), 4.43-4.27 (m, 1H), 4.16-3.98 (m, 3H), 3.95 (s, 1H), 3.71 (br d, J=14.6 Hz, 1H), 3.64-3.51 (m, 2H), 3.31 (br s, 1H), 3.01 (ddd, J=4.4, 6.0, 9.6 Hz, 1H), 2.61-2.52 (m, 2H), 2.33 (br d, J=14.8 Hz, 1H), 2.14-1.92 (m, 2H), 1.91-1.75 (m, 2H), 1.74-1.57 (m, 4H), 1.16 (d, J=16.4 Hz, 3H); LCMS (ESI, M+1): m/z=616.3.

### Example 802

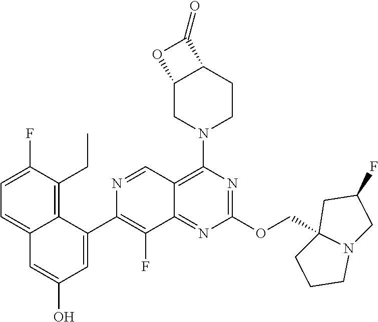 [SMILES: CCc1c(F)ccc2cc(O)cc(-c3ncc4c(N5CC[C@H]6C(=O)O[C@H]6C5)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[4.2.0]octan-7-one

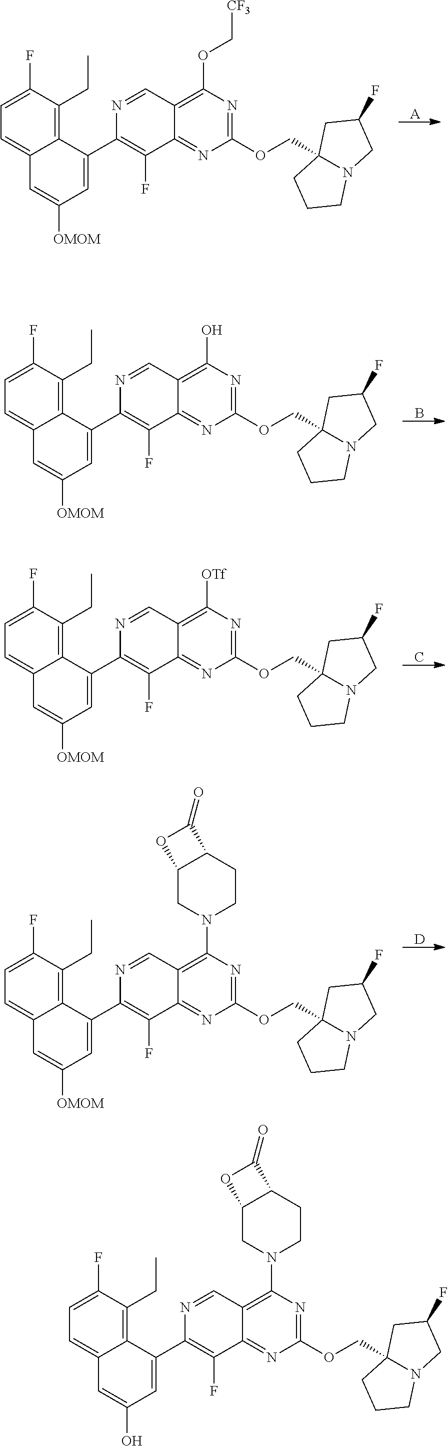

Step A: 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (1.00 g, 1.57 mmol, 1.00 eq) in H₂O (10.00 mL) and THF (10.0 mL) was added LiOH·H₂O (197 mg, 4.71 mmol, 3.00 eq). The reaction was stirred at 25° C. for 16 hrs and then quenched by 10% citric acid. The pH of the mixture was adjusted to 7 at 25° C., and extracted with ethyl acetate (10.0 mL). The organic layer was washed with brine (20.0 mL), dried over Na₂SO₄, filtered, and concentrated under reduced pressure to give the title compound (820 mg, 1.28 mmol, 81.2% yield, 86.3% purity) as a yellow solid. LCMS: Rt=0.468 min, m/z=555.2, M+H⁺

Step B: 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl trifluoromethanesulfonate. To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-ol (50.0 mg, 90.1 umol, 1.00 eq) in dichloromethane (1.00 mL) were added TEA (54.7 mg, 541 umol, 75.3 uL, 6.00 eq) and Tf₂O (61.0 mg, 216 umol, 35.7 uL, 2.40 eq). The mixture was stirred at 0° C. for 15 min. The reaction was quenched by 2 mL aq. NaHCO₃ at 25° C., and extracted with 2 mL dichloromethane twice.

The combined organic layers were washed with brine 2.00 mL, dried over Na₂SO₄, filtered, and concentrated under reduced pressure to give 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl trifluoromethanesulfonate (72 mg, crude) as a yellow oil.

Step C: 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[4.2.0]octan-7-one. A mixture of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl trifluoromethanesulfonate (120 mg, 175 umol, 1.00 eq) and cis-8-oxa-3-azabicyclo[4.2.0]octan-7-one (44.4 mg, 349 umol, 2.00 eq), TEA (53.0 mg, 524 umol, 73.0 uL, 3.00 eq) in DMF (1.00 mL) was degassed and purged with N₂ for 3 times. The mixture was stirred under N₂ atmosphere at 25° C. for 1 hour. The reaction mixture was purified by reversed-phase HPLC (0.1% FA condition) to give the title compound (34.0 mg, 51.2 umol, 14.7% yield) as a yellow oil. LCMS: Rt=0.712 min, m/z=664.5, M+H⁺

Step D: 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[4.2.0]octan-7-one. A mixture of 3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-oxa-3-azabicyclo[4.2.0]octan-7-one (20.0 mg, 30.1 umol, 1.00 eq), HCl/EtOAc (4 M, 22.6 uL, 3.00 eq) in dichloromethane (0.20 mL) was degassed and purged with N₂ for 3 times. The reaction was stirred under N₂ atmosphere at 0° C. for 1 hour. The mixture was concentrated and the residue was purified by reversed-phase HPLC (0.1% FA condition) to give the title compound (8.6 mg, 12.28 umol, 40.75% yield, 89.0% purity) as a white solid; ¹H NMR: EW39947-1-PIE (400 MHZ, CDCl₃): δ: 9.03-9.00 (m, 1H), 7.54-7.51 (m, 1H), 7.24-7.16 (m, 2H), 7.09-7.01 (m, 1H), 5.49-5.26 (m, 1H), 4.93-4.88 (m, 1H), 4.67-4.58 (m, 2H), 4.44-4.35 (m, 2H), 4.09-3.80 (m, 4H). 3.59-3.56 (m, 2H), 3.45-3.28 (m, 2H), 3.15-3.08 (m, 2H), 2.58-2.28 (m, 6H), 1.32-1.26 (m, 4H). LCMS: Rt=0.440 min, m/z=620.3, M+H⁺

### Example 803

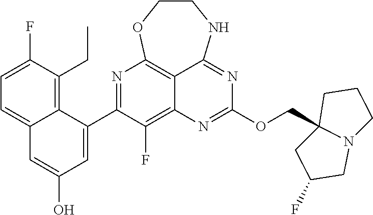 [SMILES: CCc1c(F)ccc2cc(O)cc(-c3nc4c5c(nc(OC[C@@]67CCCN6C[C@H](F)C7)nc5c3F)NCCO4)c12]

### 5-ethyl-6-fluoro-4-(4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)naphthalen-2-ol

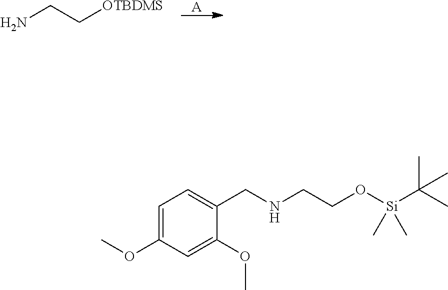

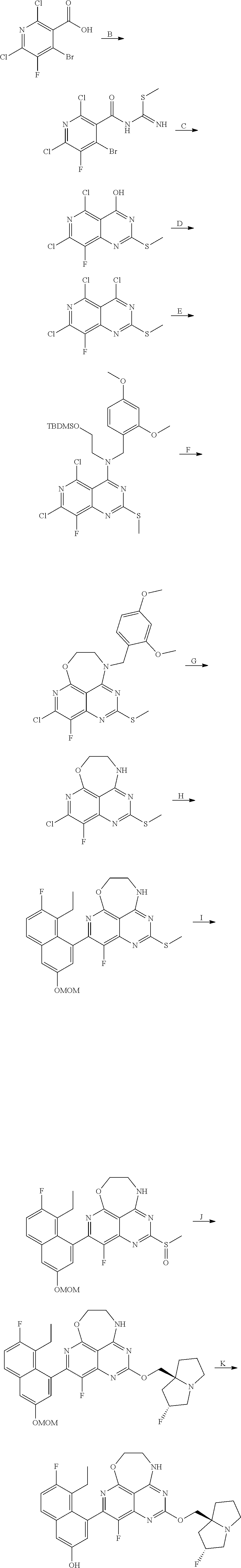

Step A. 2-((tert-butyldimethylsilyl)oxy)-N-(2,4-dimethoxybenzyl) ethan-1-amine: To a solution of 2-((tert-butyldimethylsilyl)oxy) ethan-1-amine (5.00 g, 1.0 equiv) and 2,4-dimethoxybenzaldehyde (2.37 g, 0.50 equiv) in DCE (30 mL) was added NaBH(OAc)₃ (7.25 g, 1.2 equiv) at 0° C. The reaction was stirred at 20° C. for 2 hours. The mixture was diluted with water (30 mL) and extracted with EtOAc (3×30 mL). The combined organic layers were washed with brine (40 mL), dried over anhydrous sodium sulfate, concentrated, and purified with column chromatography [SiO₂, Dichloromethane/Methanol=100/1 to 50/1] to afford the title compound (3.00 g, 31% yield) as white solid: LCMS (ESI, M+1): m/z=326.3.

Step B. methyl (4-bromo-2,6-dichloro-5-fluoronicotinoyl) carbamimidothioate: A solution of 4-bromo-2,6-dichloro-5-fluoronicotinic acid (20.0 g, 1.0 equiv) in SOCl₂ (200 mL) and DMF (1 mL) was stirred at 100° C. for 1 hour. The mixture was concentrated. The residue was dissolved in THF (200 mL). DIEA (25.2 g, 3.0 equiv) and methyl carbamimidothioate (36.2 g, 2.0 equiv) were added at 0° C. The reaction was stirred at 20° C. for 12 hours. The mixture was filtered, diluted with water (200 mL) and extracted with EtOAc (3×200 mL). The combined organic layers were washed with brine (600 mL), dried over anhydrous sodium sulfate, concentrated, and purified with column chromatography [SiO₂, Petroleum ether/Ethyl acetate=20/1 to 5/1] to afford the title compound (8.00 g, 32% yield) as yellow solid; LCMS (ESI, M+1, M+3): m/z=359.8, 361.8.

Step C. 5,7-dichloro-8-fluoro-2-(methylthio)pyrido[4,3-d]pyrimidin-4-ol: To a solution of methyl (4-bromo-2,6-dichloro-5-fluoronicotinoyl) carbamimidothioate (6.00 g, 1.0 equiv) in dioxane (40 mL) was added DIEA (6.44 g, 3.0 equiv). The reaction was stirred at 100° C. for 12 hours. The mixture was diluted with water (50 mL) and extracted with EtOAc (3×50 mL). The combined organic layers were washed with brine (100 mL), dried over anhydrous sodium sulfate, concentrated, and purified with column chromatography [SiO₂, Petroleum ether/Ethyl acetate=10/1 to 3/1] to afford the title compound (5.00 g, 93% yield) as yellow solid; LCMS (ESI, M+1): m/z=280.0.

Step D. 4,5,7-trichloro-8-fluoro-2-(methylthio)pyrido[4,3-d]pyrimidine: To a mixture of 5,7-dichloro-8-fluoro-2-(methylthio)pyrido[4,3-d]pyrimidin-4-ol (1.00 g, 1.0 equiv) and DIEA (1.38 g, 3.0 equiv) in MeCN (10 mL) was added POCl₃ (2.74 g, 5.0 equiv) at 0° C. The reaction was degassed and purged with nitrogen 3 times. The reaction was stirred at 90° C. for 2 hours. The mixture was concentrated to afford the title compound (1.00 g, crude) as yellow solid.

Step E. N-(2-((tert-butyldimethylsilyl)oxy)ethyl)-5,7-dichloro-N-(2,4-dimethoxybenzyl)-8-fluoro-2-(methylthio)pyrido[4,3-d]pyrimidin-4-amine: To a solution of 4,5,7-trichloro-8-fluoro-2-(methylthio)pyrido[4,3-d]pyrimidine (250 mg, 1.0 equiv) and DIEA (325 mg, 3.0 equiv) in DCM (5.0 mL) was added 2-((tert-butyldimethylsilyl)oxy)-N-(2,4-dimethoxybenzyl) ethan-1-amine (409 mg, 1.5 equiv) at −40° C. The reaction was stirred at −40° C. for 0.5 hours. The mixture was diluted with water (10 mL) and extracted with DCM (3×10 mL). The combined organic layers were washed with brine (20 mL), dried over anhydrous sodium sulfate, concentrated, and purified with reversed phase flash chromatography [C18, 0.1% formic acid condition] to afford the title compound (380 mg, 72% yield) as yellow oil; LCMS (ESI, M+1): m/z=587.2.

Step F. 5-chloro-10-(2,4-dimethoxybenzyl)-4-fluoro-2-(methylthio)-9,10-dihydro-8H-7-oxa-1,3,6,10-tetraazacyclohepta[de]naphthalene: To a solution of N-(2-((tert-butyldimethylsilyl)oxy)ethyl)-5,7-dichloro-N-(2,4-dimethoxybenzyl)-8-fluoro-2-(methylthio)pyrido[4,3-d]pyrimidin-4-amine (370 mg, 1.0 equiv) in DMF (8 mL) was added CsF (478 mg, 5.0 equiv). The reaction was stirred at 40° C. for 4 hours. The mixture was diluted with water (10 mL) and extracted with EtOAc (3×10 mL). The combined organic layers were washed with brine (20 mL), dried over anhydrous sodium sulfate, concentrated, and purified with reversed phase flash chromatography [C18, 0.1% formic acid condition] to afford the title compound (200 mg, 72% yield) as yellow solid; LCMS (ESI, M+1): m/z=437.2.

Step G. 5-chloro-4-fluoro-2-(methylthio)-9,10-dihydro-8H-7-oxa-1,3,6,10-tetraazacyclohepta[de]naphthalene: A solution of 5-chloro-10-(2,4-dimethoxybenzyl)-4-fluoro-2-(methylthio)-9,10-dihydro-8H-7-oxa-1,3,6,10-tetraazacyclohepta[de]naphthalene (190 mg, 1.0 equiv) in TFA (2 mL) was stirred at 80° C. for 4 hours. The pH of the mixture was adjusted to >8 with saturated NaHCO₃ aqueous (10 mL) at 0° C. and the mixture was extracted with DCM (3×10 mL). The combined organic layers were washed with brine (10 mL), dried over anhydrous sodium sulfate, and concentrated to afford the title compound (200 mg, crude) as yellow oil; LCMS (ESI, M+1): m/z=286.9.

Step H. 5-(8-ethyl-7-fluoro-3-(methoxymethoxy)naphthalen-1-yl)-4-fluoro-2-(methylthio)-9,10-dihydro-8H-7-oxa-1,3,6,10-tetraazacyclohepta[de]naphthalene: To a mixture of 5-chloro-4-fluoro-2-(methylthio)-9,10-dihydro-8H-7-oxa-1,3,6,10-tetraazacyclohepta[de]naphthalene (170 mg, 1.0 equiv), K₃PO₄ (1.5 M in H₂O, 1.19 mL, 3.0 equiv) and 2-(8-ethyl-7-fluoro-3-(methoxymethoxy)naphthalen-1-yl)-4,4,5,5-tetramethyl-1,3,2-dioxaborolane (214 mg, 1.0 equiv) in DMF (3 mL) was added CataCXium A Pd G3 (43.2 mg, 0.10 equiv). The reaction was degassed and purged with nitrogen 3 times. The reaction was stirred at 90° C. for 2 hours. The mixture was diluted with water (10 mL) and extracted with EtOAc (3× 5 mL). The combined organic layers were washed with brine (10 mL), dried over anhydrous sodium sulfate, concentrated and purified with reversed phase flash chromatography [C18, 0.1% formic acid condition] to afford the title compound (120 mg, 41% yield) as yellow solid; LCMS (ESI, M+1): m/z=485.2.

Step I. 5-(8-ethyl-7-fluoro-3-(methoxymethoxy)naphthalen-1-yl)-4-fluoro-2-(methylsulfinyl)-9,10-dihydro-8H-7-oxa-1,3,6,10-tetraazacyclohepta[de]naphthalene: To a solution of 5-(8-ethyl-7-fluoro-3-(methoxymethoxy)naphthalen-1-yl)-4-fluoro-2-(methylthio)-9,10-dihydro-8H-7-oxa-1,3,6,10-tetraazacyclohepta[de]naphthalene (110 mg, 1.0 equiv) in MeOH (0.3 mL), THF (1 mL) and H₂O (0.3 mL) was added oxone (126 mg, 0.9 equiv) at 0° C. The reaction was stirred at 20° C. for 2 hours. The mixture was diluted with water (3 mL) and extracted with EtOAc (3×3 mL). The combined organic layers were washed with brine (5 mL), dried over anhydrous sodium sulfate, concentrated to afford the title compound (110 mg, crude) as yellow solid; LCMS (ESI, M+1): m/z=501.2.

Step J. 5-(8-ethyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene: A mixture of 5-(8-ethyl-7-fluoro-3-(methoxymethoxy)naphthalen-1-yl)-4-fluoro-2-(methylsulfinyl)-9,10-dihydro-8H-7-oxa-1,3,6,10-tetraazacyclohepta[de]naphthalene (110 mg, 1.0 equiv) and ((2R,7aS)-2-fluorotetrahydro-1H-pyrrolizin-7a(5H)-yl)methanol (175 mg, 5.0 equiv) was stirred at 60° C. for 2 hours. The mixture was diluted with water (5 mL) and extracted with EtOAc (3×5 mL). The combined organic layers were washed with brine (10 mL), dried over anhydrous sodium sulfate, concentrated, and purified with reversed phase flash chromatography [C18, 0.1% formic acid condition] to afford the title compound (110 mg, 83% yield) as yellow solid; LCMS (ESI, M+1): m/z=596.3

Step K. 5-ethyl-6-fluoro-4-(4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalen-5-yl)naphthalen-2-ol: To a solution of 5-(8-ethyl-7-fluoro-3-(methoxymethoxy)naphthalen-1-yl)-4-fluoro-2-(((2R,7aS)-2-fluorotetrahydro-1H-pyrrolizin-7a(5H)-yl)methoxy)-9,10-dihydro-8H-7-oxa-1,3,6,10-tetraazacyclohepta[de]naphthalene (50.0 mg, 1.0 equiv) in MeOH (0.5 mL) was added HCl·MeOH (4 M, 0.5 mL). The reaction was stirred at 0° C. for 0.5 hours. The pH of the mixture was adjusted to pH>8 with saturated NaHCO₃ aqueous (5 mL) at 0° C. and the mixture was extracted with DCM (3×5 mL). The combined organic layers were washed with brine (10 mL), dried over anhydrous sodium sulfate, concentrated and purified with prep-HPLC [Phenomenex luna C18 150×25 mm×10 μm; A: water (FA), B: ACN; B %: 24%-44% over 10 min] to afford the title compound (20.1 mg, 43% yield, 0.41 HCOOH) as white solid; ¹H NMR (400 MHZ, METHANOL-d₄) δ=7.68-7.64 (m, 1H), 7.33-7.19 (m, 2H), 7.15-7.05 (m, 1H), 5.50-5.28 (m, 1H), 4.65-4.58 (m, 2H), 4.49-4.30 (m, 2H), 3.85-3.82 (m, 2H), 3.64-3.41 (m, 3H), 3.24-3.11 (m, 1H), 2.60-2.49 (m, 1H), 2.49-2.26 (m, 3H), 2.25-2.06 (m, 3H), 1.98-1.96 (m, 1H), 0.87 (t, J=7.2 Hz, 3H); LCMS (ESI, M+1): m/z=552.3.

### Example 804

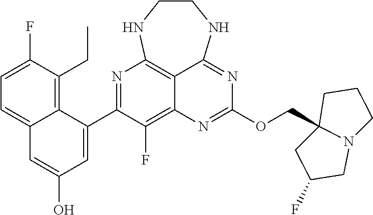 [SMILES: CCc1c(F)ccc2cc(O)cc(-c3nc4c5c(nc(OC[C@@]67CCCN6C[C@H](F)C7)nc5c3F)NCCN4)c12]

### 5-ethyl-6-fluoro-4-(4-fluoro-2-(((2R,7aS)-2-fluorotetrahydro-1H-pyrrolizin-7a(5H)-yl)methoxy)-7,8,9,10-tetrahydro-1,3,6,7,10-pentaazacyclohepta[de]naphthalen-5-yl)naphthalen-2-ol

### Example 805

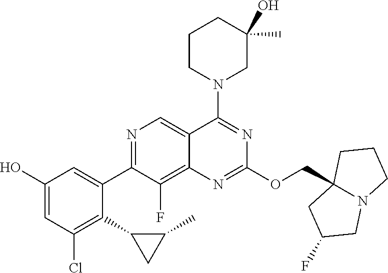 [SMILES: C[C@@H]1C[C@@H]1c1c(Cl)cc(O)cc1-c1ncc2c(N3CCC[C@@](C)(O)C3)nc(OC[C@@]34CCCN3C[C@H](F)C4)nc2c1F]

### (R)-1-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol

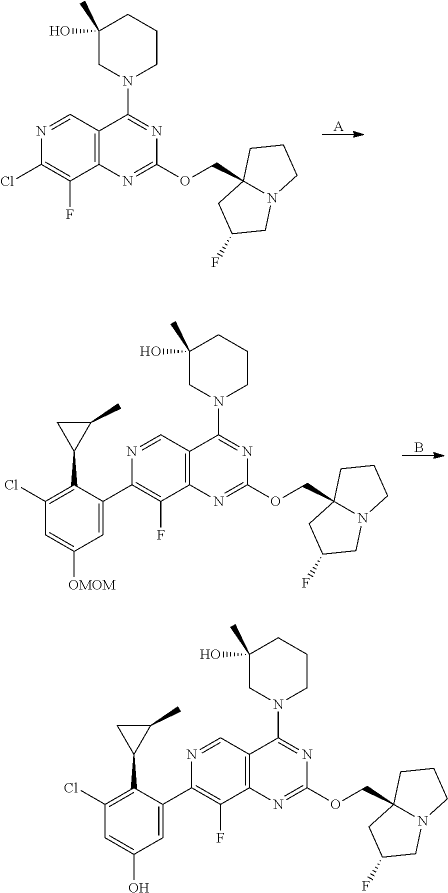

Step A. (R)-1-(7-(3-chloro-5-(methoxymethoxy)-2-((1R,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol: To a solution of (R)-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (200 mg, 1.0 equiv), 2-(3-chloro-5-(methoxymethoxy)-2-((1R,2R)-2-methylcyclopropyl)phenyl)-4,4,5,5-tetramethyl-1,3,2-dioxaborolane (186 mg, 1.2 equiv) and K₃PO₄ (1.5 M in water, 3.0 equiv) in CPME (3.0 mL) was added CataCXium A Pd G3 (32.1 mg, 0.1 equiv). The reaction was degassed and purged with nitrogen 3 times. The reaction was stirred at 90° C. for 3 hours. The mixture was diluted with water (10 mL) and extracted with ethyl acetate (2×10 mL). The combined organic layers were washed with brine (15 mL), dried over anhydrous sodium sulfate, concentrated and purified with reversed phase flash chromatography [C18, 0.1% formic acid condition] to afford the title compound (100 mg, 33% yield) as white solid; LCMS (ESI, M+1): m/z=644.3.

Step B. (R)-1-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol To a solution of (R)-1-(7-(3-chloro-5-(methoxymethoxy)-2-((1R,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (100 mg, 1.0 equiv) in MeCN (1.5 mL) was added HCl·dioxane (4 M, 100 equiv) at 0° C. The reaction was stirred at 0° C. for 0.5 hours. The mixture was concentrated and purified with prep-HPLC [column: Waters Xbridge 150×25 mm×5 μm; A: water (NH₄HCO₃), B: ACN, B %: 51%-81% over 10 min] to afford the title compound (29.0 mg, 31% yield) as off-white solid; ¹H NMR (400 MHZ, METHANOL-d₄) δ=9.19 (d, J=13.2 Hz, 1H), 7.00 (d, J=2.8 Hz, 1H), 6.82 (d, J=2.4 Hz, 1H), 5.41-5.18 (m, 1H), 4.62-4.46 (m, 2H), 4.37-4.18 (m, 3H), 3.61 (br d, J=13.2 Hz, 1H), 3.50-3.39 (m, 1H), 3.30-3.12 (m, 3H), 3.01 (dt, J=5.6, 9.2 Hz, 1H), 2.40-2.06 (m, 4H), 2.05-1.69 (m, 7H), 1.28 (d, J=2.0 Hz, 3H), 1.18-1.02 (m, 1H), 0.74-0.52 (m, 4H); LCMS (ESI, M+1): m/z=600.4.

### Example 806

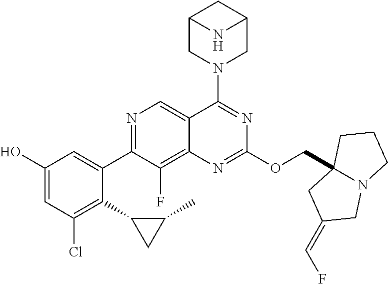

### 3-(4-(3,8-diazabicyclo[3.2.1]octan-3-yl)-8-fluoro-2-(((S,Z)-2-(fluoromethylene)tetrahydro-1H-pyrrolizin-7a(5H)-yl)methoxy)pyrido[4,3-d]pyrimidin-7-yl)-5-chloro-4-(cis-2-methylcyclopropyl)phenol

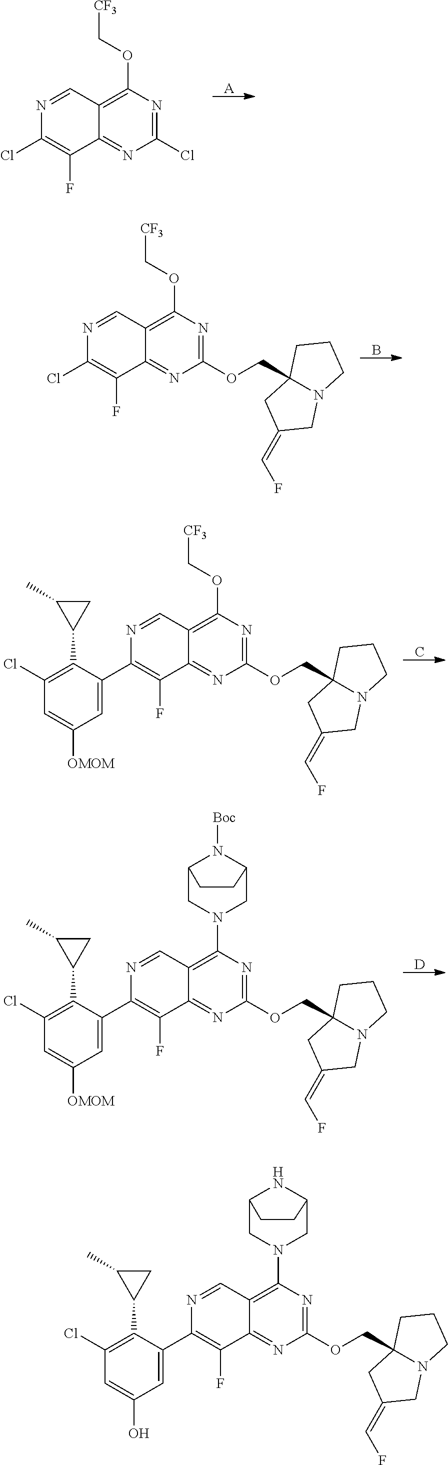

Step A. (S,Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (200 mg, 1.0 equiv) and (S,Z)-(2-(fluoromethylene)tetrahydro-1H-pyrrolizin-7a(5H)-yl)methanol (130 mg, 1.2 equiv) in THF (5 mL) were added DIEA (327 mg, 4.0 equiv) and 4 Å molecular sieves (30.0 mg). The reaction was stirred at 40° C. for 12 hours. The mixture was diluted with water (20 mL) and extracted with ethyl acetate (2×20 mL). The combined organic layers were washed with brine (20 mL), dried over anhydrous sodium sulfate, concentrated and purified with column chromatography [SiO₂, petroleum ether/ethyl acetate=1/0 to 5/1] to afford the title compound (250 mg, 72% yield) as yellow solid; LCMS (ESI, M+1): m/z=450.9.

Step B. 7-(3-chloro-5-(methoxymethoxy)-2-(cis-2-methylcyclopropyl)phenyl)-8-fluoro-2-(((S,Z)-2-(fluoromethylene)tetrahydro-1H-pyrrolizin-7a(5H)-yl)methoxy)-4-(2,2,2-trifluoroethoxy)pyrido[4,3-d]pyrimidine: To a solution of (S,Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine (200 mg, 1.0 equiv) and 2-(3-chloro-5-(methoxymethoxy)-2-((1S,2R)-2-methylcyclopropyl)phenyl)-4,4,5,5-tetramethyl-1,3,2-dioxaborolane (156 mg, 1.0 equiv) in methoxycyclopentane (4 mL) and water (1 mL) were added Cs₂CO₃ (434 mg, 3.0 equiv) and CataCXium A Pd G3 (64.6 mg, 0.2 equiv). The reaction was degassed and purged with N₂ for 3 times. The reaction was stirred at 100° C. for 2 hours under N₂ atmosphere. The mixture was diluted with water (30 mL) and extracted with ethyl acetate (2× 30 mL). The combined organic layers were washed with brine (30 mL), dried over anhydrous sodium sulfate, concentrated and purified with prep-TLC [SiO₂, Petroleum ether/Ethyl acetate=1/1] to afford the title compound (55.0 mg, 16% yield) as yellow solid; LCMS (ESI, M+1): m/z=641.2.

Step C. tert-butyl(1R,5S)-3-(7-(3-chloro-5-(methoxymethoxy)-2-(cis-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3,8-diazabicyclo[3.2.1]octane-8-carboxylate: To a solution of 7-(3-chloro-5-(methoxymethoxy)-2-(cis-2-methylcyclopropyl)phenyl)-8-fluoro-2-(((S,Z)-2-(fluoromethylene)tetrahydro-1H-pyrrolizin-7a(5H)-yl)methoxy)-4-(2,2,2-trifluoroethoxy)pyrido[4,3-d]pyrimidine (50.0 mg, 1.0 equiv) and tert-butyl(1R,5S)-3,8-diazabicyclo[3.2.1]octane-8-carboxylate (24.8 mg, 1.5 equiv) in DMF (1 mL) were added DIEA (30.2 mg, 3.0 equiv) and 4 Å molecular sieves (20.0 mg). The reaction was stirred at 40° C. for 10 hours. The mixture was diluted with water (10 mL) and extracted with ethyl acetate (2×10 mL). The combined organic layers were washed with brine (10 mL), dried over anhydrous sodium sulfate, concentrated and purified with prep-TLC [SiO₂, petroleum ether/ethyl acetate=0/1] to afford the title compound (30.0 mg, 43% yield) as yellow solid; LCMS (ESI, M+1): m/z=753.4.

Step D. 3-(4-(cis-3,8-diazabicyclo[3.2.1]octan-3-yl)-8-fluoro-2-(((S,Z)-2-(fluoromethylene)tetrahydro-1H-pyrrolizin-7a(5H)-yl)methoxy)pyrido[4,3-d]pyrimidin-7-yl)-5-chloro-4-(cis-2-methylcyclopropyl)phenol: To a solution of tert-butyl(1R,5S)-3-(7-(3-chloro-5-(methoxymethoxy)-2-(cis-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3,8-diazabicyclo[3.2.1]octane-8-carboxylate (27.0 mg, 1.0 equiv) in MeCN (2 mL) was added HCl·dioxane (2 mL). The reaction was stirred at 0° C. for 0.5 hours. The mixture was concentrated, dissolved in MeOH (2 mL), adjusted to pH=7 with solid NaHCO₃ and filtered. The filtrate was concentrated and purified with prep-HPLC [column: Phenomenex C18 75×30 mm×3 um; mobile phase: water (NH₄HCO₃)-ACN; gradient: 40%-70% B over 10 minutes] to afford the title compound (9.06 mg, 41% yield) as white solid, ¹H NMR (400 MHZ, METHANOL-d₄) δ=9.04 (s, 1H), 7.00 (d, J=2.4 Hz, 1H), 6.82 (d, J=2.4 Hz, 1H), 6.77-6.50 (m, 1H), 4.67-4.53 (m, 2H), 4.36-4.21 (m, 2H), 3.84 (br d, J=14.8 Hz, 1H), 3.75-3.63 (m, 4H), 3.46 (br d, J=15.2 Hz, 1H), 3.22-3.06 (m, 1H), 2.80-2.64 (m, 2H), 2.44 (br d, J=15.6 Hz, 1H), 2.19-1.67 (m, 10H), 1.23-1.01 (m, 1H), 0.65 (br d, J=6.0 Hz, 4H); LCMS (ESI, M+1): m/z=609.3.

### Example 807

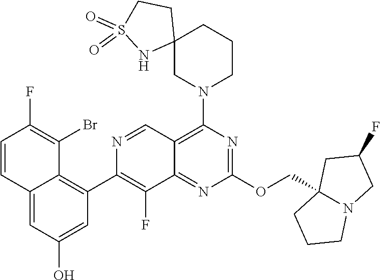 [SMILES: O=S1(=O)CCC2(CCCN(c3nc(OC[C@@]45CCCN4C[C@H](F)C5)nc4c(F)c(-c5cc(O)cc6ccc(F)c(Br)c56)ncc34)C2)N1]

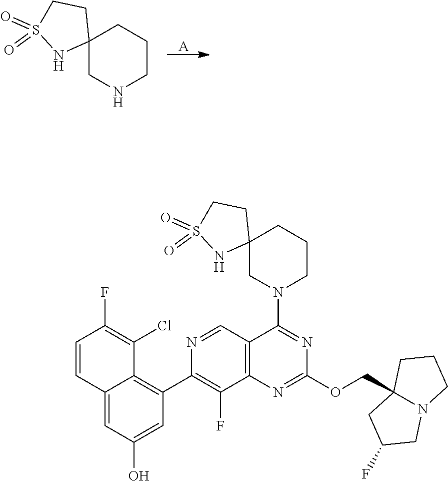

Step A. 7-(7-(8-chloro-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,7-diazaspiro[4.5]decane 2,2-dioxide: To a mixture of 7-(8-chloro-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (50.0 mg, 66.2 umol, 1.00 eq.), 2-thia-1,7-diazaspiro[4.5]decane 2,2-dioxide (37.8 mg, 3.00 equiv), 4 Å molecular sieve (50 mg) in DMF (0.5 mL) was added K₃PO₄ (70.3 mg, 5.00 equiv) The reaction was stirred at 60° C. for 12 hours. The mixture was filtered, concentrated and purified by prep-HPLC [Waters Xbridge 150×25 mm×5 μm; A: water (NH₃·H₂O); B: ACN, B %: 30%-60% over 9 min] followed by prep-HPLC [Phenomenex luna C18 150×25 mm×10 μm; A: water (FA); B: ACN, B %: 13%-43% over 10 min] to give title compound (15.1 mg, 15% yield) as a yellow solid; ¹H NMR (400 MHz, DMSO-d6) δ=9.10 (d, J=7.6 Hz, 1H), 8.17 (d, J=2.8 Hz, 1H), 7.99-7.89 (m, 1H), 7.56 (dt, J=3.6, 8.8 Hz, 1H), 7.43 (d, J=2.0 Hz, 1H), 7.42-7.33 (m, 1H), 7.24-7.15 (m, 1H), 5.40-5.16 (m, 1H), 4.23-3.97 (m, 4H), 3.83-3.70 (m, 1H), 3.69-3.49 (m, 2H), 3.25-3.17 (m, 2H), 3.15-3.06 (m, 2H), 3.06-2.98 (m, 1H), 2.91-2.77 (m, 1H), 2.31-2.21 (m, 1H), 2.20-2.10 (m, 2H), 2.03-1.89 (m, 3H), 1.88-1.68 (m, 5H); LCMS (ESI, M+1): m/z=689.3.

### Example 808

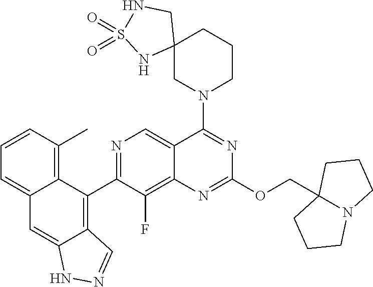 [SMILES: Cc1cccc2cc3[nH]ncc3c(-c3ncc4c(N5CCCC6(CNS(=O)(=O)N6)C5)nc(OCC56CCCN5CCC6)nc4c3F)c12]

### 7-(8-fluoro-7-(5-methyl-1H-benzo[f]indazol-4-yl)-2-((tetrahydro-1H-pyrrolizin-7a(5H)-yl)methoxy)pyrido[4,3-d]pyrimidin-4-yl)-2-thia-1,3,7-triazaspiro[4.5]decane 2,2-dioxide

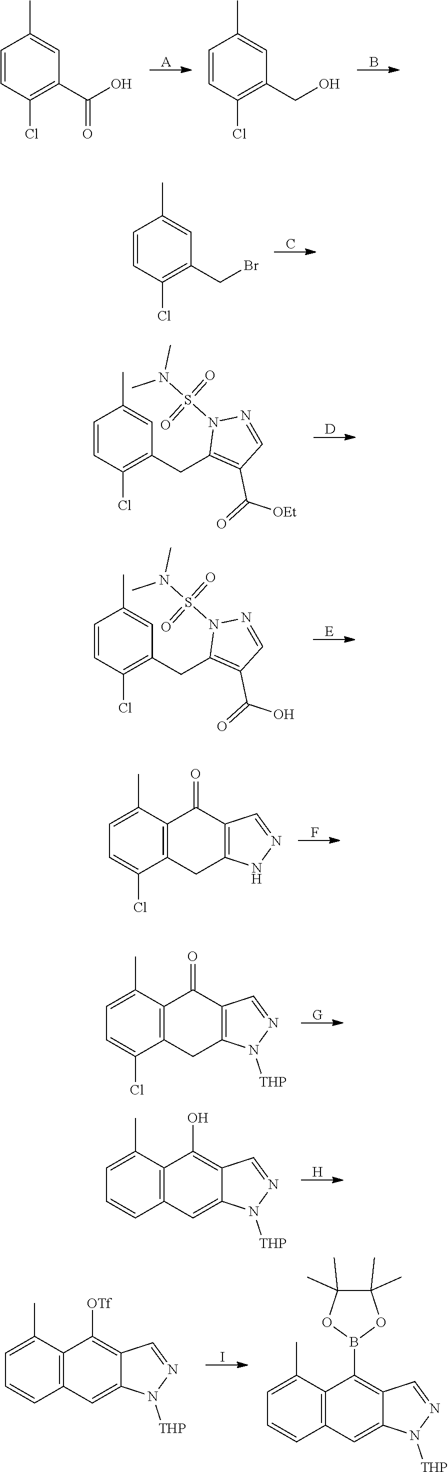

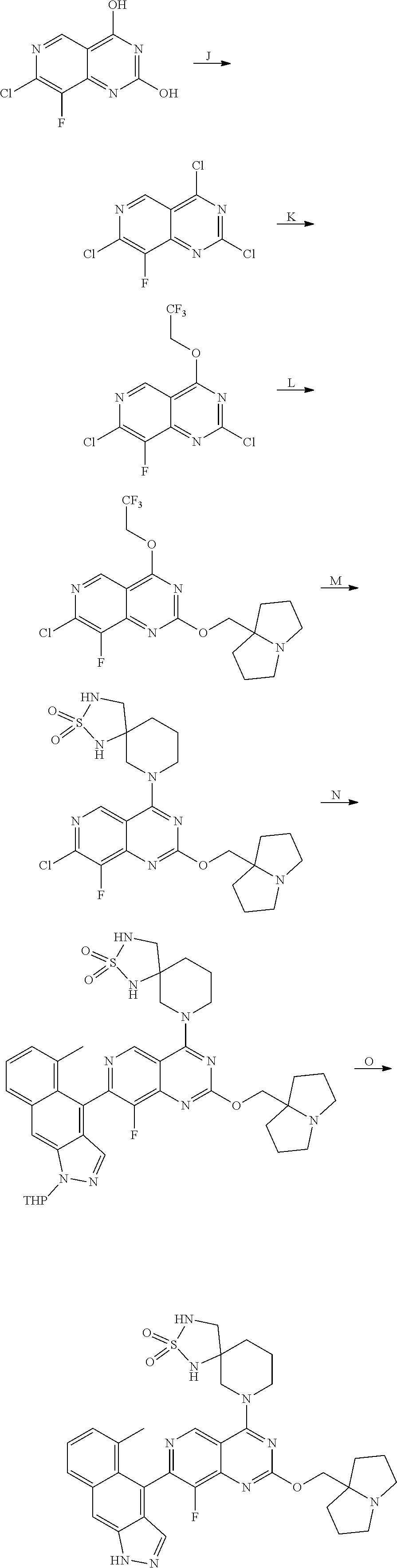

Step A. (2-chloro-5-methylphenyl)methanol: To a mixture of 2-chloro-5-methylbenzoic acid (45.0 g, 1.0 equiv) in THF (500 mL) was added BH₃·Me₂S (10 M, 158 mL, 6.0 equiv) at 0° C. The reaction was stirred at 20° C. for 12 hours. The mixture was quenched with MeOH (500 ml) and concentrated to afford the title compound (40 g, crude) as yellow solid.

Step B. 2-(bromomethyl)-1-chloro-4-methylbenzene: To a mixture of (2-chloro-5-methylphenyl)methanol (40.0 g, 1.0 equiv) in DCM (400 mL) was added PBr₃ (138 g, 2.0 equiv) at 0° C. The reaction was stirred at 0° C. for 1 hour. The mixture was concentrated under vacuum. The mixture was diluted with water (100 mL) at 0° C. The pH value of the mixture was adjusted to 7 with saturated sodium bicarbonate. The mixture was extracted with ethyl acetate (2×300 mL). The combined organic layers were washed with brine (300 mL), dried over anhydrous sodium sulfate, filtered, concentrated and purified with column chromatography (SiO₂, petroleum ether/ethyl acetate=10/1) to afford the title compound (47.1 g, 83% yield) as yellow solid; ¹H NMR (400 MHZ, METHANOL-d4) δ=7.30 (d, J=1.2 Hz, 1H), 7.26 (d, J=8.0 Hz, 1H), 7.10 (dd, J=1.6, 8.0 Hz, 1H), 4.59 (s, 2H), 2.30 (s, 3H).

Step C. ethyl 5-(2-chloro-5-methylbenzyl)-1-(N,N-dimethylsulfamoyl)-1H-pyrazole-4-carboxylate: To a solution of ethyl 1-(N,N-dimethylsulfamoyl)-1H-pyrazole-4-carboxylate (74.6 g, 1.0 equiv) in THF (500 mL) were added LDA (2 M, 196 mL, 1.3 equiv) and HMPA (70.3 g, 1.3 equiv) at −78° C. in 0.5 hour. The reaction was stirred at −78° C. for 0.5 hour. 2-(bromomethyl)-1-chloro-4-methylbenzene (53.0 g, 0.8 equiv) was added into above mixture. The reaction was stirred at −78° C. for 0.5 hour and 20° C. for 3 hours. The mixture was quenched with water (500 mL) at 0° C. and extracted with ethyl acetate (1000 mL). The combined organic layers were washed with brine (500 mL), dried over anhydrous sodium sulfate, filtered, concentrated and purified with reversed phase flash chromatography [water (FA 0.1%)/acetonitrile] to afford the title compound (17.0 g, 14% yield) as yellow solid; LCMS (ESI, M+1): m/z=386.1.

Step D. 5-(2-chloro-5-methylbenzyl)-1-(N,N-dimethylsulfamoyl)-1H-pyrazole-4-carboxylic acid: To a mixture of ethyl 5-(2-chloro-5-methylbenzyl)-1-(N,N-dimethylsulfamoyl)-1H-pyrazole-4-carboxylate (23.0 g, 1.0 equiv) in dioxane (160 mL) was added a solution of NaOH (47.7 g, 20.0 equiv) in H₂O (160 mL). The reaction was stirred at 90° C. for 2 hours. The mixture was extracted with ethyl acetate (2×200 mL). The combined organic layers were washed with brine (200 mL), dried over anhydrous sodium sulfate, filtered, concentrated to afford the title compound (27.0 g, crude) as yellow solid.

Step E. 8-chloro-5-methyl-1H-benzo[f]indazol-4 (9H)-one: A mixture of 5-(2-chloro-5-methylbenzyl)-1-(N,N-dimethylsulfamoyl)-1H-pyrazole-4-carboxylic acid (36.6 g, 1.0 equiv) in CF₃SO₃H (300 mL) was stirred at 90° C. for 2 hours. The mixture was poured into ice water (600 mL) and adjusted to pH˜8 with NaHCO₃ aqueous solution. The mixture was extracted with ethyl acetate (3×1000 ml). The combined organic layers were washed with brine (1000 mL), dried over anhydrous sodium sulfate, filtered, concentrated and purified with reversed phase flash chromatography [water (FA 0.1%)/acetonitrile] to afford the title compound (20.1 g, 83% yield) as yellow solid; LCMS (ESI, M+1): m/z=233.1.

Step F. 8-chloro-5-methyl-1-(tetrahydro-2H-pyran-2-yl)-1H-benzo[f]indazol-4 (9H)-one: To a mixture of 8-chloro-5-methyl-1H-benzo[f]indazol-4 (9H)-one (5.40 g, 1.0 equiv) and TsOH·H₂O (44.1 mg, 0.01 equiv) in THF (50 mL) was added DHP (3.90 g 2.0 equiv). The reaction was stirred at 20° C. for 2 hours. The mixture was quenched with water (40 mL) and extracted with ethyl acetate (2×50 mL). The combined organic layers were washed with brine (50 mL), dried over anhydrous sodium sulfate, filtered, concentrated and purified with reversed phase flash chromatography [water (FA 0.1%)/acetonitrile] to afford the title compound (3.62 g, 49% yield) as yellow solid.

Step G. 5-methyl-1-(tetrahydro-2H-pyran-2-yl)-1H-benzo[f]indazol-4-ol: To a mixture of 8-chloro-5-methyl-1-(tetrahydro-2H-pyran-2-yl)-1H-benzo[f]indazol-4 (9H)-one (3.60 g, 1.0 equiv) and NaHCO₃ (2.86 g, 3.0 equiv) in MeOH (50 mL) was added Pd/C (360 mg, 5% purity) under N₂ atmosphere. The reaction was degassed and purged with H₂ 3 times. The reaction was stirred under H₂ (15 Psi) at 20° C. for 5 hours. The mixture was diluted with water (50 mL) and concentrated under vacuum to remove MeOH. The mixture was extracted with EtOAc (3× 50 mL). The combined organic layers were washed with brine (50 mL), dried over anhydrous sodium sulfate and concentrated under vacuum to afford the title compound (2.29 g, 60% yield) as yellow solid; LCMS (ESI, M+1): m/z=282.8.

Step H. 5-methyl-1-(tetrahydro-2H-pyran-2-yl)-1H-benzo[f]indazol-4-yl trifluoromethanesulfonate: To a mixture of 5-methyl-1-(tetrahydro-2H-pyran-2-yl)-1H-benzo[f]indazol-4-ol (2.7 g, 1.0 equiv), DIEA (4.9 g, 4.0 equiv) and 4 Å molecular sieve (100 mg) in DCM (20 mL) was added Tf₂O (5.40 g, 2.0 equiv) at −40° C. The reaction was stirred at −40° C. for 0.5 hour. The mixture was quenched with water (20 mL) and extracted with dichloromethane (2× 30 mL). The combined organic layers were washed with brine (30 mL), dried over anhydrous sodium sulfate, filtered, concentrated and purified with column chromatography (SiO₂, petroleum ether/ethyl acetate=10/1) to afford the title compound (1.08 g, 27% yield) as yellow solid; LCMS (ESI, M+1): m/z=415.0.

Step I. 5-methyl-1-(tetrahydro-2H-pyran-2-yl)-4-(4,4,5,5-tetramethyl-1,3,2-dioxaborolan-2-yl)-1H-benzo[f]indazole: To a solution of 5-methyl-1-(tetrahydro-2H-pyran-2-yl)-1H-benzo[f]indazol-4-yl trifluoromethanesulfonate (550 mg, 1.0 equiv), 4,4,5,5-tetramethyl-1,3,2-dioxaborolane (1.70 g, 10.0 equiv) and TEA (403 mg, 3.0 equiv) in MeCN (5 mL) was added Pd(dppf)Cl₂ (97.1 mg, 0.1 equiv). The reaction was degassed and purged with nitrogen 3 times. The reaction was stirred at 80° C. for 12 hours. The mixture was concentrated and purified with prep-HPLC (column: Phenomenex luna C18 150*25 mm*10 um; mobile phase: [water (FA)-ACN]; B %: 54%-84%, 8 min) to afford the title compound (229 mg, 36% yield) as yellow solid; LCMS (ESI, M+1): m/z=393.1.

Step J. 2,4,7-trichloro-8-fluoropyrido[4,3-d]pyrimidine; To a mixture of POCl₃ (17.8 g, 5.0 equiv) in toluene (50 mL) were added 7-chloro-8-fluoropyrido[4,3-d]pyrimidine-2,4-diol (5.00 g, 1.0 equiv) and DIEA (6.00 g, 2.0 equiv). The reaction was stirred at 110° C. for 24 hours. The mixture was concentrated under vacuum. The residue was diluted with iced saturated NaHCO₃ solution (50 ml) and brought the pH to 8. The mixture was filtered and filter cake was dissolved in ethyl acetate (100 ml). The mixture washed with saturated NaHCO₃ aqueous solution (50 ml) and brine (50 ml), dried over anhydrous sodium sulfate, concentrated under vacuum to afford the title compound (4.9 g, crude) as yellow solid.

Step K. 2,7-dichloro-8-fluoro-4-(2,2,2-trifluoroethoxy)pyrido[4,3-d]pyrimidine: To a solution of 2,2,2-trifluoroethanol (991 mg, 1.0 equiv) in THF (8 mL) was added t-BuONa (2 M, 4.95 mL, 1.0 equiv) at 25° C. The reaction was stirred at 25° C. for 1 hour. The above mixture was added to a solution of 2,4,7-trichloro-8-fluoropyrido[4,3-d]pyrimidine (2.5 g, 1.0 equiv) in THF (20 mL) at −40° C. The reaction was stirred at −40° C. for 1 hour. The mixture was quenched with water (20 mL) and extracted with ethyl acetate (2× 50 mL). The combined organic layers were washed with brine (20 mL), dried over anhydrous sodium sulfate, filtered, concentrated to afford the title compound (3.28 g, crude) as yellow solid; LCMS (ESI, M+1): m/z=315.7.

Step L. 7-chloro-8-fluoro-2-((hexahydro-1H-pyrrolizin-7a-yl)methoxy)-4-(2,2,2-trifluoroethoxy)pyrido[4,3-d]pyrimidine: To a mixture of 2,7-dichloro-8-fluoro-4-(2,2,2-trifluoroethoxy)pyrido[4,3-d]pyrimidine (4.00 g, 1.0 equiv) and Na₂CO₃ (4.02 g, 3.0 equiv) in THF (40 mL) was added (hexahydro-1H-pyrrolizin-7a-yl)methanol (3.57 g, 2.0 equiv). The reaction was stirred at 60° C. for 3 hours. The mixture was quenched with water (30 mL) and extracted with ethyl acetate (2×50 mL). The combined organic layers were washed with brine (50 mL), dried over anhydrous sodium sulfate, filtered, concentrated and purified with reversed phase flash chromatography [water (FA 0.1%)/acetonitrile] to afford the title compound (800 mg, 15% yield) as yellow solid; LCMS (ESI, M+1): m/z=421.2.

Step M. 7-(7-chloro-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide: To a mixture of 7-chloro-8-fluoro-2-((hexahydro-1H-pyrrolizin-7a-yl)methoxy)-4-(2,2,2-trifluoroethoxy)pyrido[4,3-d]pyrimidine (200 mg, 1.0 equiv), DIEA (614 mg, 10 equiv) and 4 Å molecular sieve (20 mg) in DMF (2 mL) was added 2-thia-1,3,7-triazaspiro[4.5]decane 2,2-dioxide (227 mg, 2.5 equiv). The reaction was stirred at 60° C. for 2 hours. The mixture was filtered and concentrated and purified with reversed phase flash chromatography [water (FA 0.1%)/acetonitrile] to afford the title compound (200 mg, 81% yield) as yellow solid; LCMS (ESI, M+1): m/z=512.1.

Step N. 7-(8-fluoro-2-((hexahydro-1H-pyrrolizin-7a-yl)methoxy)-7-(5-methyl-1-(tetrahydro-2H-pyran-2-yl)-1H-benzo[f]indazol-4-yl)pyrido[4,3-d]pyrimidin-4-yl)-2-thia-1,3,7-triazaspiro[4.5]decane 2,2-dioxide: To a mixture of 7-(7-chloro-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide (50.0 mg, 1.0 equiv), 5-methyl-1-(tetrahydro-2H-pyran-2-yl)-4-(4,4,5,5-tetramethyl-1,3,2-dioxaborolan-2-yl)-1H-benzo[f]indazole (38.3 mg, 1.0 equiv) and K₃PO₄ (62.2 mg, 3.0 equiv) in Methoxycyclopentane (1 mL) and H₂O (0.3 mL) was added cataCXium® A Pd G3 (7.11 mg 0.1 equiv). The reaction was degassed and purged with nitrogen 3 times. The reaction was stirred at 90° C. for 3 hours. The mixture was quenched with water (3 mL) and extracted with ethyl acetate (2×5 mL). The combined organic layers were washed with brine (5 mL), dried over anhydrous sodium sulfate, filtered, concentrated and purified with column chromatography (SiO₂, Dichloromethane:Methanol=0/1) to afford the title compound (17.8 mg, 23% yield) as yellow solid. LCMS (ESI, M+1): m/z=742.8.

Step O. 7-(8-fluoro-2-((hexahydro-1H-pyrrolizin-7a-yl)methoxy)-7-(5-methyl-1H-benzo[f]indazol-4-yl)pyrido[4,3-d]pyrimidin-4-yl)-2-thia-1,3,7-triazaspiro[4.5]decane 2,2-dioxide: A mixture of 7-(8-fluoro-2-((hexahydro-1H-pyrrolizin-7a-yl)methoxy)-7-(5-methyl-1-(tetrahydro-2H-pyran-2-yl)-1H-benzo[f]indazol-4-yl)pyrido[4,3-d]pyrimidin-4-yl)-2-thia-1,3,7-triazaspiro[4.5]decane 2,2-dioxide (25.0 mg 1.0 equiv) in TFA (770 mg, 0.5 mL) was stirred at 25° C. for 0.5 hour. The mixture was concentrated under vacuum. The residue was diluted with water (1 mL). The mixture was adjusted to pH=8 with saturated NaHCO₃ (10 mL) and extracted with ethyl acetate (3×2 ml). The combined organic layers were washed with brine (4 mL), dried over anhydrous sodium sulfate, filtered, concentrated and purified with prep-HPLC (column: Phenomenex luna C18 150*25 mm*10 um; mobile phase: [water (FA)-ACN]; B %: 12%-42%, 10 min) to afford the title compound (3.40 mg 15% yield, HCOOH salt) as yellow solid. ¹H NMR (400 MHZ, METHANOL-d4) δ=9.26-9.15 (m, 1H), 8.54 (br s, 1H), 8.19 (d, J=4.0 Hz, 1H), 7.95 (d, J=8.4 Hz, 1H), 7.88-7.66 (m, 1H), 7.39-7.33 (m, 1H), 7.19 (br d, J=6.4 Hz, 1H), 4.66-4.49 (m, 4H), 3.84 (d, J=13.6 Hz, 1H), 3.67-3.36 (m, 4H), 3.22-3.15 (m, 1H), 3.14-3.01 (m, 2H), 2.32-2.20 (m, 2H), 2.15 (s, 3H), 2.13-2.04 (m, 5H), 2.03-1.76 (m, 5H). LCMS (ESI, M+1): m/z=658.3.

### Example 809

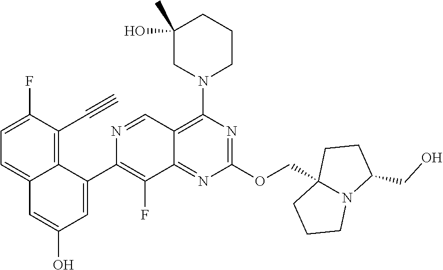 [SMILES: C#Cc1c(F)ccc2cc(O)cc(-c3ncc4c(N5CCC[C@@](C)(O)C5)nc(OC[C@@]56CCCN5[C@@H](CO)CC6)nc4c3F)c12]

### (R)-1-(7-(8-ethynyl-7-fluoro-3-hydroxynaphthalen-1-yl)-8-fluoro-2-(((3R,7aS)-3-(hydroxymethyl)tetrahydro-1H-pyrrolizin-7a(5H)-yl)methoxy)pyrido[4,3-d]pyrimidin-4-yl)-3-methylpiperidin-3-ol

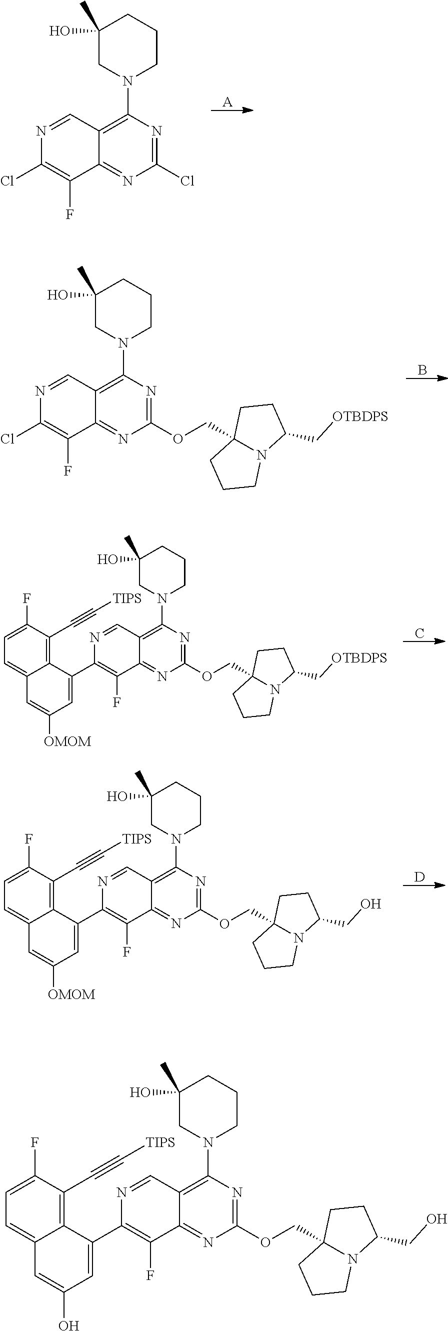

Step A. (R)-1-(2-(((3R,7aS)-3-(((tert-butyldiphenylsilyl)oxy)methyl) hexahydro-1H-pyrrolizin-7a-yl)methoxy)-7-chloro-8-fluoropyrido[4,3-d]pyrimidin-4-yl)-3-methylpiperidin-3-ol: To a mixture of ((3R,7aS)-3-(((tert-butyldiphenylsilyl)oxy)methyl) hexahydro-1H-pyrrolizin-7a-yl)methanol (618 mg, 1.0 equiv), t-BuONa (2 M, 1.5 mL, 2.0 equiv) and 4 Å molecular sieve (100 mg) in toluene (5.0 mL) was added (R)-1-(2,7-dichloro-8-fluoropyrido[4,3-d]pyrimidin-4-yl)-3-methylpiperidin-3-ol (500 mg, 1.0 equiv). The reaction was stirred at 0° C. for 1 hour under N₂ atmosphere. The mixture was diluted with water (30 mL), extracted with ethyl acetate (3×30 mL), washed with brine (2× 30 mL), dried, concentrated and purified with column chromatography [SiO₂, petroleum ether/ethyl acetate=2/1 to methylene chloride/methanol=10/1] to afford the title compound (860 mg, 54% yield,) as yellow solid; LCMS (ESI, M+1): m/z=704.5.

Step B. (R)-1-(2-(((3R,7aS)-3-(((tert-butyldiphenylsilyl)oxy)methyl) hexahydro-1H-pyrrolizin-7a-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3-methylpiperidin-3-ol: To a mixture of (R)-1-(2-(((3R,7aS)-3-(((tert-butyldiphenylsilyl)oxy)methyl) hexahydro-1H-pyrrolizin-7a-yl)methoxy)-7-chloro-8-fluoropyrido[4,3-d]pyrimidin-4-yl)-3-methylpiperidin-3-ol (50.0 mg, 1.0 equiv), Ad₂nBu Pd G₃ (5.17 mg, 0.1 equiv) and Cs₂CO₃ (69.4 mg, 3.0 equiv) in H₂O (0.2 mL) and CPME (1.0 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (43.7 mg, 1.2 equiv). The reaction was stirred at 80° C. for 2 hours under N₂ atmosphere. The mixture was diluted with water (20 mL), extracted with DCM (3×20 mL), washed with brine (10 mL), dried, concentrated and purified with prep-TLC (SiO₂, methylene chloride/methanol=10:1) to afford the title compound (50.0 mg, 67% yield) as white solid; LCMS (ESI, M+1): m/z=1054.5.

Step C. (R)-1-(7-(8-ethynyl-7-fluoro-3-(methoxymethoxy)naphthalen-1-yl)-8-fluoro-2-(((3R,7aS)-3-(hydroxymethyl) hexahydro-1H-pyrrolizin-7a-yl)methoxy)pyrido[4,3-d]pyrimidin-4-yl)-3-methylpiperidin-3-ol: To a solution of (R)-1-(2-(((3R,7aS)-3-(((tert-butyldiphenylsilyl)oxy)methyl) hexahydro-1H-pyrrolizin-7a-yl)methoxy)-8-fluoro-7-(7-fluoro-3-(methoxymethoxy)-8-((triisopropylsilyl)ethynyl)naphthalen-1-yl)pyrido[4,3-d]pyrimidin-4-yl)-3-methylpiperidin-3-ol (50.0 mg, 1.0 equiv) in DMF (1.0 mL) was added CsF (259 mg, 30 equiv). The reaction was stirred at 20° C. for 2 hours. The mixture was diluted with water (20 mL), extracted with DCM (3×20 mL), washed with brine (20 mL), dried and concentrated to afford the title compound (30.0 mg, crude) as white solid; LCMS (ESI, M+1): m/z=660.3.

Step D. (R)-1-(7-(8-ethynyl-7-fluoro-3-hydroxynaphthalen-1-yl)-8-fluoro-2-(((3R,7aS)-3-(hydroxymethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethynyl-7-fluoro-3-(methoxymethoxy)naphthalen-1-yl)-8-fluoro-2-(((3R,7aS)-3-(hydroxymethyl) hexahydro-1H-pyrrolizin-7a-yl)methoxy)pyrido[4,3-d]pyrimidin-4-yl)-3-methylpiperidin-3-ol (30.0 mg, 1.0 equiv) in ACN (1.0 mL) was added HCl·dioxane (4 M, 1.0 mL). The reaction was stirred at 0° C. for 1 hour. The mixture was quenched with NaHCO₃ at 0° cand adjusted pH to 7, filtered and purified with prep-HPLC [column: Phenomenex luna C18 150×25 mm×10 um; mobile phase: water (FA)-ACN; B %: 10%-40%, 10 min] to afford the title compound (10.0 mg, 36% yield,) as orange solid; ¹H NMR (400 MHZ, METHANOL-d4) δ=9.26-9.03 (m, 1H), 8.54 (s, 1H), 7.94-7.79 (m, 1H), 7.40-7.17 (m, 3H), 4.70-4.58 (m, 1H), 4.55-4.44 (m, 2H), 4.40-4.25 (m, 1H), 3.76-3.49 (m, 4H), 3.46-3.33 (m, 2H), 3.28-3.13 (m, 2H), 2.39-2.27 (m, 1H), 2.25-2.15 (m, 1H), 2.14-2.02 (m, 4H), 2.00-1.88 (m, 3H), 1.88-1.70 (m, 3H), 1.38-1.19 (m, 3H); LCMS (ESI, M+1): m/z=616.3.

### Example 810

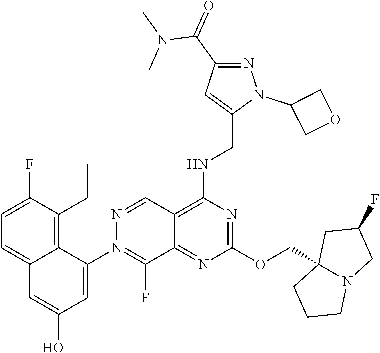

### 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1-(oxetan-3-yl)-1H-pyrazole-3-carboxamide

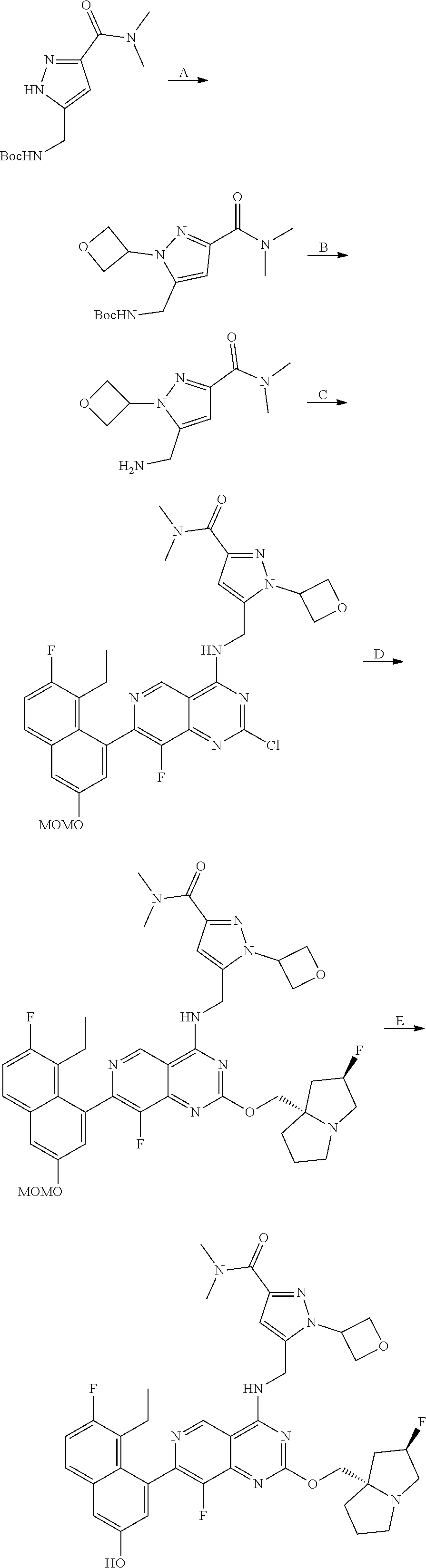

Step A. tert-butyl((3-(dimethylcarbamoyl)-1-(oxetan-3-yl)-1H-pyrazol-5-yl)methyl)carbamate: To a mixture of tert-butyl N-[[3-(dimethylcarbamoyl)-1H-pyrazol-5-yl]methyl]carbamate (686 mg, 2.0 equiv) in ACN (5 mL) was added Cs₂CO₃ (1.21 g, 2.0 equiv). The reaction was stirred at 80° C. for 12 hours. The mixture was filtered and purified by reversed phase flash chromatography [C18, 0.1% formic acid condition] to afford the title compound (500 mg, 82% yield) as yellow solid; LCMS (ESI, M+1): m/z=324.9.

Step B. 5-(aminomethyl)-N,N-dimethyl-1-(oxetan-3-yl)-1H-pyrazole-3-carboxamide: To a solution of tert-butyl((3-(dimethylcarbamoyl)-1-(oxetan-3-yl)-1H-pyrazol-5-yl)methyl)carbamate (70 mg, 1.0 equiv) in DCM (120 μL) was added TFA (369 mg, 15 equiv). The reaction was stirred at 20° C. for 0.5 hours. The mixture was concentrated to afford the title compound (73 mg, CF₃COOH) as yellow oil.

Step C. 5-(((2-chloro-7-(8-ethyl-7-fluoro-3-(methoxymethoxy)naphthalen-1-yl)-8-fluoropyrido[4,3-d]pyrimidin-4-yl)amino)methyl)-N,N-dimethyl-1-(oxetan-3-yl)-1H-pyrazole-3-carboxamide: To a mixture of 5-(aminomethyl)-N,N-dimethyl-1-(oxetan-3-yl)-1H-pyrazole-3-carboxamide (73.0 mg, 0.9 equiv, CF₃COOH) and DIEA (651 mg, 21 equiv) in DCM (1 mL) were added 2,4-dichloro-7-(8-ethyl-7-fluoro-3-(methoxymethoxy)naphthalen-1-yl)-8-fluoropyrido[4,3-d]pyrimidine (108 mg, 1.0 equiv) and 4 Å molecular sieve (50.0 mg, 1.0 equiv) at −40° C. The reaction was stirred at −40° C. for 15 minutes. The mixture was diluted with water (5 mL) and extracted with DCM (3×5 mL). The combined organic layers were dried over anhydrous sodium sulfate, concentrated and purified by prep-HPLC [Welch Xtimate C18 150×25 mm×5 μm; A: water (NH₄HCO₃), B: ACN; B %: 35%-65% over 2 min] to afford the title compound (30.0 mg, 19% yield) as white solid; LCMS (ESI, M+1): m/z=638.6.

Step D. 5-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1-(oxetan-3-yl)-1H-pyrazole-3-carboxamide: To a mixture of 5-(((2-chloro-7-(8-ethyl-7-fluoro-3-(methoxymethoxy)naphthalen-1-yl)-8-fluoropyrido[4,3-d]pyrimidin-4-yl)amino)methyl)-N,N-dimethyl-1-(oxetan-3-yl)-1H-pyrazole-3-carboxamide (200 mg, 1.0 equiv) and ((2R,7aS)-2-fluorohexahydro-1H-pyrrolizin-7a-yl)methanol (100 mg, 2.0 equiv) in dioxane (2 mL) was added DIEA (406 mg, 10 equiv). The reaction was stirred at 90° C. for 12 hours. The mixture was diluted with water (5 mL) and extracted with EtOAc (3×5 mL). The combined organic layers were washed with brine (5 mL), dried over anhydrous sodium sulfate, concentrated and purified by reversed phase flash chromatography [C18, 0.1% formic acid condition] followed by purified by prep-HPLC [Waters xbridge 150×25 mm×10 μm; A: water (NH₄HCO₃), B: ACN; B %: 39%-69% over 9 min] to afford the title compound (30 mg, 12% yield) as yellow solid; LCMS (ESI, M+1): m/z=761.4.

Step E. 5-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1-(oxetan-3-yl)-1H-pyrazole-3-carboxamide: To a mixture of 5-(((7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-N,N-dimethyl-1-(oxetan-3-yl)-1H-pyrazole-3-carboxamide (25.0 mg, 1.0 equiv) in DCM (2.5 mL) was added TFA (770 mg, 205 equiv). The reaction was stirred at 0° C. for 1 hour. The mixture was concentrated by blowing of nitrogen, dissolved in methanol (1 mL), neutralized with solid NaHCO₃, filtered and purified by prep-HPLC [Phenomenex luna C 18×150×25 mm×10 μm; A: water (FA), B: ACN; B %: 15%-45% over 60 min] to afford the title compound (6.25 mg, 24% yield, HCOOH) as off-white solid; ¹H NMR (400 MHZ, methanol-d₄) δ=9.19-9.13 (m, 1H), 8.501 (s, 1H), 7.71-7.64 (m, 1H), 7.35-7.29 (m, 1H), 7.28-7.21 (m, 1H), 7.03 (d, J=2.4 Hz, 1H), 6.78-6.72 (m, 1H), 6.04-5.95 (m, 1H), 5.49-5.30 (m, 1H), 5.20-5.12 (m, 2H), 5.09-5.01 (m, 2H), 4.93 (s, 2H), 4.50-4.36 (m, 2H), 3.58-3.39 (m, 6H), 3.23-3.15 (m, 1H), 3.14-3.08 (m, 3H), 2.53-2.31 (m, 3H), 2.29-2.19 (m, 1H), 2.18-2.06 (m, 3H), 2.05-1.92 (m, 1H), 0.77 (t, J=7.2 Hz, 3H); LCMS (ESI, M+1): m/z=717.5.

### Example 811

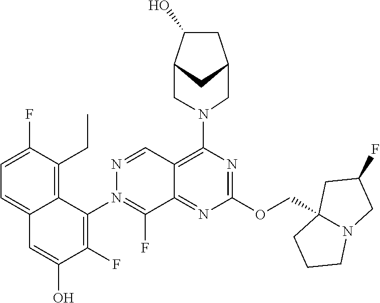

### (1R,5R,6R)-3-(7-(8-ethyl-2,7-difluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

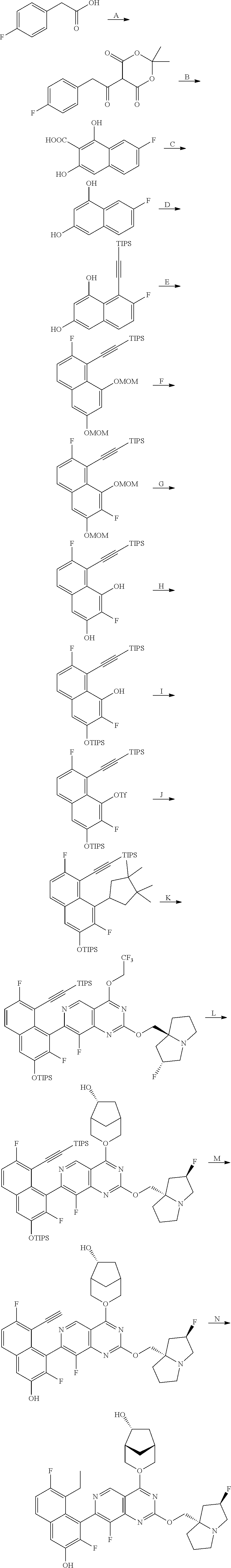

Step A. 5-(2-(4-fluorophenyl) acetyl)-2,2-dimethyl-1,3-dioxane-4,6-dione: To a mixture of 2-(4-fluorophenyl) acetic acid (250 g, 1.0 equiv) and 2,2-dimethyl-1,3-dioxane-4,6-dione (257 g, 1.1 equiv) in acetonitrile (1.25 L) were added DMAP (16.9 g, 0.09 equiv) and DIPEA (451 g, 2.1 equiv) below 30° C. for 1 hour. Pivaloyl chloride (215 g, 1.1 equiv) was added into above mixture dropwise below 40° C. for 1 hour. The reaction was stirred at 45° C. for 3 hours. The mixture was cooled to 0° C. and adjusted pH to 5 with 4 N HCl (5.00 L). The mixture was stirred at 0° C. for 1 hour. The mixture was diluted with H₂O (15 L) and the pH of the mixture was adjusted to 2 with 4N HCl. The mixture was filtered. The filter cake was washed with H₂O until the pH of filtrate was 5˜6. The solid was dried under reduced pressure to afford the tittle compound (500 g, crude) as white solid; ¹H NMR (400 MHZ, chloroform-d) δ=7.36 (dd, J=5.6, 8.4 Hz, 2H), 7.01 (t, J=8.4 Hz, 2H), 4.38 (s, 2H), 1.72 (s, 6H).

Step B. 7-fluoro-1,3-dihydroxy-2-naphthoic acid: To CF₃SO₃H (2.04 kg, 7.8 equiv) in reaction bottle was added 5-(2-(4-fluorophenyl) acetyl)-2,2-dimethyl-1,3-dioxane-4,6-dione (490 g, 1.0 equiv) in portions below 30° C. The reaction was stirred at 20° C. for 2 hours. The mixture was poured into ice water (30 L) and filtered. The filter cake was washed with H₂O until the pH of the filtrate was 3˜4 to afford the tittle compound (500 g, crude) as brown solid.

Step C. 7-fluoronaphthalene-1,3-diol: A mixture of 7-fluoro-1,3-dihydroxy-2-naphthoic acid (375 g, crude) in H₂O (1.8 L) and acetonitrile (1.8 L) was stirred at 78° C. for 13 hours. The mixture was concentrated to remove acetonitrile. The residue was diluted with H₂O (1 L) and saturated NaHCO₃ aqueous (0.3 L) and extracted with ethyl acetate (4× 0.5 L). The combined organic layers were washed with saturated NaHCO₃ aqueous (0.5 L), H₂O (0.5 L) and brine (0.5 L), dried over anhydrous sodium sulfate and concentrated to give a residue. The solid was dispersed in n-heptane (0.8 L) and the mixture was stirred for 1 hour. The mixture was filtered and the solid was dried under reduced pressure to afford the tittle compound (145 g, 60% yield over three steps) as light red solid; ¹H NMR (400 MHz, DMSO-d₆) δ=10.18 (s, 1H), 9.48 (s, 1H), 7.65-7.56 (m, 2H), 7.23 (dt, J=2.8, 8.8 Hz, 1H), 6.64 (d, J=1.6 Hz, 1H), 6.56 (d, J=1.6 Hz, 1H).

Step D. 7-fluoro-8-((triisopropylsilyl)ethynyl)naphthalene-1,3-diol: To a mixture of 7-fluoronaphthalene-1,3-diol (173 g, 1.0 equiv) and (bromoethynyl)triisopropylsilane (266 g, 1.05 equiv) in dioxane (1.5 L) were added KOAc (191 g, 2.0 equiv) and [Ru(p-cymene)Cl₂]₂ (17.8 g, 0.03 equiv). The reaction was degassed and purged with nitrogen 3 times. The reaction was stirred at 100° C. for 3.5 hours. The mixture was filtered through a pad of Celite. The filter cake was washed with ethyl acetate (4×500 mL). The filtrate was concentrated and dissolved in ethyl acetate (3 L). The solution was washed with saturated NaHCO₃ aqueous (0.5 L) and brine (0.2 L), dried over anhydrous sodium sulfate, concentrated, and purified by column chromatography [SiO₂, petroleum ether/ethyl acetate=15/1 to 10/1] to give a residue. The residue was dispersed in n-heptane (0.5 L) and stirred for 1 hour. The mixture was filtered, and the filter cake was washed with n-heptane (0.5 L). The solid was dried under reduced pressure to afford the tittle compound (204 g, 56% yield) as light yellow solid; ¹H NMR (400 MHZ, DMSO-d6) δ=10.04 (s, 1H), 9.58 (s, 1H), 7.63 (dd, J=5.6, 9.2 Hz, 1H), 7.25 (t, J=9.2 Hz, 1H), 6.63 (d, J=2.4 Hz, 1H), 6.58 (d, J=2.0 Hz, 1H), 1.13 (s, 21H).

Step E. ((2-fluoro-6,8-bis(methoxymethoxy)naphthalen-1-yl)ethynyl)triisopropylsilane: To a solution of 7-fluoro-8-((triisopropylsilyl)ethynyl)naphthalene-1,3-diol (50.0 g, 1.0 equiv) and DIEA (90.1 g, 5.0 equiv) in dichloromethane (500 mL) was added bromo(methoxy) methane (69.7 g, 4.0 equiv) at 0° C. The reaction was stirred at 15° C. for 10 hours. The mixture was diluted with water (200 mL). The organic layer was concentrated and purified with column chromatography [SiO₂, Petroleum ether/Ethyl acetate=1/0 to 20/1] to afford the title compound (58.0 g, 93% yield) as yellow oil; ¹H NMR (400 MHZ, CHLOROFORM-d) δ=7.61 (dd, J=5.6, 9.0 Hz, 1H), 7.21 (t, J=8.8 Hz, 1H), 7.04 (s, 2H), 5.33 (s, 2H), 5.26 (s, 2H), 3.55 (s, 3H), 3.52 (s, 3H), 1.20 (s, 21H); LCMS (ESI, M+1): m/z=447.3.

Step F. ((2,7-difluoro-6,8-bis(methoxymethoxy)naphthalen-1-yl)ethynyl)triisopropylsilane: To a solution of ((2-fluoro-6,8-bis(methoxymethoxy)naphthalen-1-yl)ethynyl)triisopropylsilane (6.70 g, 1.0 equiv) and TMEDA (1.92 g, 1.1 equiv) in THF (100 mL) was added n-BuLi (2.5 M in THF, 2.0 equiv) at 0° C. The reaction was stirred at 0° C. for 1 hour. To the mixture was added a mixture of NFSI (5.20 g, 1.1 equiv) in THF (30 mL) at −60° C. The reaction was stirred at 15° C. for 2 hours. The mixture was quenched with water (50 mL) and extracted with ethyl acetate (2×100 mL). The combined organic layers were concentrated and purified by column chromatography [SiO₂, Petroleum ether/Ethyl acetate=100/1 to 15/1] to afford the title compound (1.90 g, 27% yield) as yellow solid; ¹H NMR (400 MHZ, CHLOROFORM-d) 8-7.64 (dd, J=5.6, 8.8 Hz, 1H), 7.32 (d, J=7.6 Hz, 1H), 7.20 (t, J=8.8 Hz, 1H), 5.33 (s, 2H), 5.24 (s, 2H), 3.67 (s, 3H), 3.56 (s, 3H), 1.21-1.18 (m, 21H); LCMS (ESI, M+1): m/z=465.3.

Step G. 2,7-difluoro-8-((triisopropylsilyl)ethynyl)naphthalene-1,3-diol: To a solution of ((2,7-difluoro-6,8-bis(methoxymethoxy)naphthalen-1-yl)ethynyl)triisopropylsilane (3.00 g, 1.0 equiv) in MeOH (8 mL) was added HCl·MeOH (4 M, 10 equiv) at 0° C. The reaction was stirred at 0° C. for 1 hour. The mixture was concentrated. The residue was diluted with water (10 mL). The mixture was adjusted to pH=7 with saturated sat. NaHCO₃ (10 mL). The mixture was extracted with ethyl acetate (2× 30 mL), concentrated, and purified by column chromatography [SiO₂, Petroleum ether/Ethyl acetate=10/1 to 4/1] to afford the title compound (2.10 g, 82% yield) as yellow oil; ¹H NMR (400 MHZ, CHLOROFORM-d) δ=9.11 (s, 1H), 7.60 (dd, J=5.6, 9.2 Hz, 1H), 7.15 (t, J=8.8 Hz, 1H), 6.92 (d, J=8.0 Hz, 1H), 5.87 (br d, J=3.2 Hz, 1H), 1.24-1.16 (m, 21H); LCMS (ESI, M−1): m/z=377.1.

Step H. 2,7-difluoro-8-((triisopropylsilyl)ethynyl)-3-((triisopropylsilyl)oxy)naphthalen-1-ol: To a solution of 2,7-difluoro-8-((triisopropylsilyl)ethynyl)naphthalene-1,3-diol (2.10 g, 1.0 equiv) and DIEA (1.44 g, 2.0 equiv) in dichloromethane (10 mL) was added chlorotriisopropylsilane (1.18 g, 1.1 equiv) at 0° C. The reaction was stirred at 20° C. for 1 hour. The mixture was concentrated and purified with column chromatography [SiO₂, Petroleum ether/Ethyl acetate=100/1 to 15/1] to afford the title compound (2.20 g, 74% yield) as yellow oil; ¹H NMR (400 MHZ, CHLOROFORM-d) δ=8.97 (s, 1H), 7.58 (dd, J=5.6, 9.2 Hz, 1H), 7.13 (t, J=8.8 Hz, 1H), 6.86 (d, J=7.6 Hz, 1H), 1.22-1.11 (m, 42H); LCMS (ESI, M+1): m/z=533.3.

Step I. 2,7-difluoro-8-((triisopropylsilyl)ethynyl)-3-((triisopropylsilyl)oxy)naphthalen-1-yl trifluoromethanesulfonate: To a solution of 2,7-difluoro-8-((triisopropylsilyl)ethynyl)-3-((triisopropylsilyl)oxy)naphthalen-1-ol (2.20 g, 1.0 equiv) and DIEA (1.60 g, 3.0 equiv) in dichloromethane (20 mL) was added Tf₂O (1.63 g, 1.4 equiv) at −40° C. The reaction was stirred at −40° C. for 0.5 hours. The mixture was concentrated and purified by column chromatography [SiO₂, Petroleum ether/Ethyl acetate=1/0 to 20/1] to afford the title compound (2.20 g, 80% yield) as yellow oil; ¹H NMR (400 MHZ, CHLOROFORM-d) δ=7.62 (dd, J=5.2, 9.0 Hz, 1H), 7.31-7.28 (m, 1H), 7.28-7.25 (m, 1H), 1.22-1.10 (m, 42H).

Step J. ((3,6-difluoro-4-(4,4,5,5-tetramethyl-1,3,2-dioxaborolan-2-yl)-5-((triisopropylsilyl)ethynyl)naphthalen-2-yl)oxy)triisopropylsilane: To a solution of 2,7-difluoro-8-((triisopropylsilyl)ethynyl)-3-((triisopropylsilyl)oxy)naphthalen-1-yl trifluoromethanesulfonate (1.20 g, 1.0 equiv), 4,4,5,5-tetramethyl-1,3,2-dioxaborolane (924 mg, 4.0 equiv) and TEA (730 mg, 4.0 equiv) in acetonitrile (20 mL) was added Pd(dppf)Cl₂ (132 mg, 0.1 equiv). The reaction was degassed and purged with nitrogen 3 times. The reaction was stirred at 80° C. for 2 hours. The mixture was quenched with methanol (0.5 mL), concentrated, and purified by column chromatography [SiO₂, Petroleum ether/Ethyl acetate=100/1 to 15/1] to afford the title compound (700 mg, 54% yield) as yellow solid; ¹H NMR (400 MHZ, CHLOROFORM-d) δ=7.57 (dd, J=5.6, 9.2 Hz, 1H), 7.22 (d, J=8.8 Hz, 1H), 7.16 (t, J=8.8 Hz, 1H), 1.48 (s, 12H), 1.19-1.16 (m, 21H), 1.15-1.11 (m, 21H).

Step K. 7-(2,7-difluoro-8-((triisopropylsilyl)ethynyl)-3-((triisopropylsilyl)oxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine: To a mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (246 mg, 1.2 equiv), ((3,6-difluoro-4-(4,4,5,5-tetramethyl-1,3,2-dioxaborolan-2-yl)-5-((triisopropylsilyl)ethynyl)naphthalen-2-yl)oxy)triisopropylsilane (300 mg, 1.0 equiv) and Cs₂CO₃ (1.5 M in water, 3.0 equiv) in methoxycyclopentane (2.5 mL) was added CataCXium A Pd G3 (34.0 mg, 0.1 equiv). The reaction was degassed and purged with nitrogen 3 times. The reaction was stirred at 90° C. for 3 hours. The mixture was diluted with water (10 mL) and extracted with ethyl acetate (2×15 mL). The combined organic layers were concentrated and purified by column chromatography [SiO₂, Petroleum ether/Ethyl acetate=10/1 to 1/1] to afford the title compound (120 mg, 21 yield) as yellow solid; LCMS (ESI, M+1): m/z=919.5.

Step L. (1R,5R,6R)-3-(7-(2,7-difluoro-3-hydroxy-8-((triisopropylsilyl)ethynyl)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a mixture of 7-(2,7-difluoro-8-((triisopropylsilyl)ethynyl)-3-((triisopropylsilyl)oxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (120 mg, 1.0 equiv), (1R,5R,6R)-3-azabicyclo[3.2.1]octan-6-ol (24.9 mg, 1.5 equiv) and 4 Å molecular sieve (30 mg) in DMF (0.8 mL) was added DIEA (50.6 mg, 3.0 equiv). The mixture was stirred at 40° C. for 36 hours. The mixture was filtered. The filtrate was diluted with water (10 mL) and extracted with ethyl acetate (2×15 mL). The combined organic layers were concentrated to afford the title compound (200 mg, crude) as yellow oil; LCMS (ESI, M+1): m/z=790.4.

Step M. (1R,5R,6R)-3-(7-(8-ethynyl-2,7-difluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of (1R,5R,6R)-3-(7-(2,7-difluoro-3-hydroxy-8-((triisopropylsilyl)ethynyl)naphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (200 mg, 1.0 equiv) in DMF (1 mL) was added CsF (384 mg, 10 equiv). The reaction was stirred at 40° C. for 0.5 hours. The mixture was filtered and purified with reversed phase flash chromatography [C18, 0.1% formic acid condition] to afford the title compound (45.0 mg, 56% yield over two steps) as yellow solid; LCMS (ESI, M+1): m/z=634.3.

Step N. (1R,5R,6R)-3-(7-(8-ethyl-2,7-difluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a solution of (1R,5R,6R)-3-(7-(8-ethynyl-2,7-difluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (45.0 mg, 1.0 equiv) in MeOH (5 mL) was added Pd/C (20 mg, 10% purity) under N₂. The reaction was degassed and purged with H₂ 3 times. The reaction was stirred under H₂ (15 psi) at 20° C. for 0.5 hours. The mixture was filtered and concentrated and purified by prep-HPLC [column: Unisil 3-100 C18 Ultra 150×50 mm×3 μm; A: water (FA), B: ACN, B %: 17%-47% over 7 min] to afford the title compound (8.76 mg, 19% yield, HCOOH salt) as white solid; ¹H NMR (400 MHZ, METHANOL-d₄) δ=9.35-9.22 (m, 1H), 7.68 (dd, J=5.6, 8.8 Hz, 1H), 7.47 (d, J=9.2 Hz, 1H), 7.23 (t, J=9.2 Hz, 1H), 5.50-5.29 (m, 1H), 5.07-4.97 (m, 1H), 4.83-4.70 (m, 1H), 4.46-4.41 (m, 1H), 4.37-4.28 (m, 2H), 3.91-3.73 (m, 1H), 3.60-3.36 (m, 4H), 3.21-3.07 (m, 1H), 2.52-2.17 (m, 7H), 2.14-1.88 (m, 5H), 1.82 (br s, 1H), 1.39 (br d, J=13.6 Hz, 1H), 0.78 (td, J=7.2, 10.8 Hz, 3H); LCMS (ESI, M+1): m/z=638.3.

### Example 812

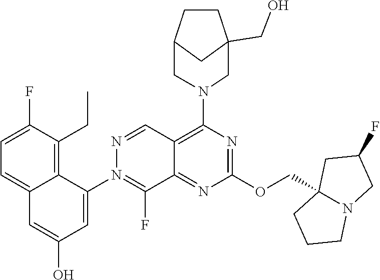

### 5-ethyl-6-fluoro-4-(8-fluoro-2-(((2R,7aS)-2-fluorotetrahydro-1H-pyrrolizin-7a(5H)-yl)methoxy)-4-(1-(hydroxymethyl)-3-azabicyclo[3.2.1]octan-3-yl)pyrido[4,3-d]pyrimidin-7-yl)naphthalen-2-ol

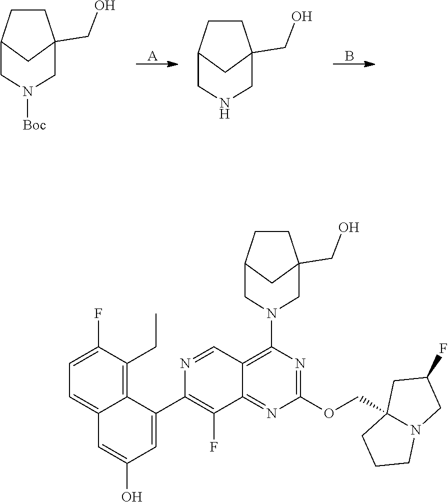

Step A. 3-azabicyclo[3.2.1]octan-1-ylmethanol: To a solution of tert-butyl 1-(hydroxymethyl)-3-azabicyclo[3.2.1]octane-3-carboxylate (100 mg, 1.0 equiv) in MeCN (1 mL) was added HCl·dioxane (4 M, 2.00 mL, 19 equiv). The reaction was stirred at 25° C. for 1 hour. The mixture was concentrated to afford the title compound (50.0 mg, 85.45% yield, hydrochloride).

Step B. 5-ethyl-6-fluoro-4-(8-fluoro-2-(((2R,7aS)-2-fluorohexahydro-1H-pyrrolizin-7a-yl)methoxy)-4-(1-(hydroxymethyl)-3-azabicyclo[3.2.1]octan-3-yl)pyrido[4,3-d]pyrimidin-7-yl)naphthalen-2-ol: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (140 mg, 1.0 equiv) in DMF (2 mL) was added 3-azabicyclo[3.2.1]octan-1-ylmethanol hydrochloride (50.0 mg, 1.5 equiv), DIEA (152 mg, 5.0 equiv) and 4 Å molecular sieve (50.0 mg). The reaction was stirred at 40° C. for 12 hours. The mixture was diluted with water (15 mL) and extracted with ethyl acetate (3×5 mL). The combined organic layers were washed with brine (5 mL), dried over sodium sulfate, concentrated and purified by prep-HPLC [column: Phenomenex luna 150×25 mm×10 μm; A: water (FA), B: ACN; B %: 15%-45% over 10 min] to afford the title compound (43.0 mg, 27% yield, HCOOH); ¹H NMR (400 MHZ, CHLOROFORM-d) δ=8.96-8.84 (m, 1H), 7.45 (br s, 1H), 7.17-6.78 (m, 3H), 5.40-5.16 (m, 1H), 4.62-4.20 (m, 4H), 3.56-2.96 (m, 10H), 2.53-2.09 (m, 6H), 2.06-1.89 (m, 3H), 1.57-1.16 (m, 5H), 0.78 (br d, J=5.6 Hz, 3H); LCMS (ESI, M+1): m/z=634.3.

### Example 813

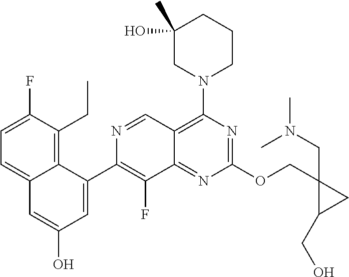 [SMILES: CCc1c(F)ccc2cc(O)cc(-c3ncc4c(N5CCC[C@@](C)(O)C5)nc(OCC5(CN(C)C)CC5CO)nc4c3F)c12]

### (3R)-1-(2-((1-((dimethylamino)methyl)-2-(hydroxymethyl)cyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

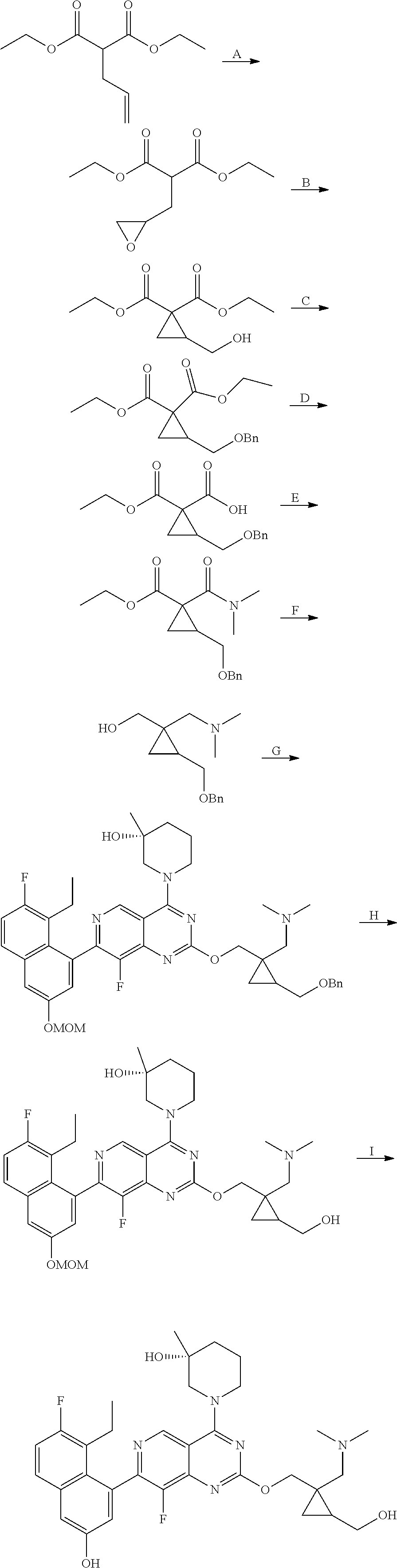

Step A. diethyl 2-(oxiran-2-ylmethyl) malonate: To a mixture of diethyl 2-allylpropanedioate (10.0 g, 1.0 equiv) in DCM (100 mL) was added m-CPBA (12.9 g, 80% purity, 1.2 equiv). The mixture was stirred at 25° C. for 15 hours under N₂ atmosphere. The mixture was filtered and the filtrate was concentrated under vacuum. The residue was re-crystallized from petroleum ether (100 mL) at 25° C. and the solid was triturated with saturated NaHCO₃ aqueous solution (100 mL) to afford the title compound (10.4 g, 85% yield) as a yellow oil. ¹H NMR (400 MHz, CHLOROFORM-d) δ=4.23-4.09 (m, 4H), 3.47 (dd, J=6.0, 8.8 Hz, 1H), 3.01-2.89 (m, 1H), 2.71 (t, J=4.4 Hz, 1H), 2.46 (dd, J=2.4, 4.8 Hz, 1H), 2.28-2.13 (m, 1H), 2.02-1.86 (m, 1H), 1.21 (dt, J=4.0, 7.2 Hz, 6H).

Step B. diethyl 2-(hydroxymethyl)cyclopropane-1,1-dicarboxylate: To a mixture of diethyl 2-(oxiran-2-ylmethyl) malonate (5.00 g, 1.0 equiv) in THF (50 mL) was added Mg(ClO₄)₂ (5.16 g, 1.0 equiv). The mixture was stirred at 60° C. for 2 hours under N₂ atmosphere. The mixture was poured into ice-water (50 mL) and extracted with ethyl acetate (3×50 mL). The combined organic phases were washed with brine (2×50 mL), dried with anhydrous Na₂SO₄, filtered, concentrated under vacuum and purified with flash silica gel chromatography (ISCO®; 80 g SepaFlash® Silica Flash Column, Eluent of 0˜50% Ethyl acetate/Petroleum ethergradient@75 mL/min) to afford the title compound (1.10 g, 22% yield) as a white solid. ¹H NMR (400 MHZ, CHLOROFORM-d) δ=4.32-4.17 (m, 4H), 3.96 (dd, J=5.2, 12.4 Hz, 1H), 3.27 (dd, J=9.6, 12.4 Hz, 1H), 2.15 (ddt, J=5.2, 7.2, 9.2 Hz, 1H), 1.55 (dd, J=5.2, 9.2 Hz, 1H), 1.36-1.21 (m, 9H).

Step C. diethyl 2-((benzyloxy)methyl)cyclopropane-1,1-dicarboxylate: To a mixture of diethyl 2-(hydroxymethyl)cyclopropane-1,1-dicarboxylate (500 mg, 1.0 equiv) and DIEA (598 mg, 2.0 equiv) in DMF (5 mL) was added BnBr (791 mg, 2.0 equiv). The mixture was stirred at 110° C. for 12 hours under N₂ atmosphere. The mixture was poured into ice-water (5 mL) and extracted with ethyl acetate (10 mL). The organic phase was washed with brine (10 mL), dried over anhydrous Na₂SO₄, filtered, concentrated under vacuum and purified with flash silica gel chromatography (ISCO®; 4 g SepaFlash® Silica Flash Column, Eluent of 0˜10% Ethyl acetate/Petroleum ethergradient@20 mL/min) to afford the title compound (490 mg, 69% yield) as a white solid. ¹H NMR (400 MHZ, CHLOROFORM-d) δ=7.36-7.28 (m, 5H), 4.45 (br d, J=8.8 Hz, 2H), 4.28-4.08 (m, 4H), 3.53 (dq, J=6.4, 10.8 Hz, 2H), 2.34-2.19 (m, 1H), 1.55 (dd, J=4.8, 7.6 Hz, 1H), 1.44 (dd, J=4.8, 9.2 Hz, 1H), 1.26 (q, J=7.2 Hz, 6H).

Step D. 2-((benzyloxy)methyl)-1-(ethoxycarbonyl)cyclopropanecarboxylic acid: To a mixture of diethyl 2-((benzyloxy)methyl)cyclopropane-1,1-dicarboxylate (420 mg, 1.0 equiv) in EtOH (6 mL) was added NaOH aqueous solution (1 M, 1.51 mL, 1.1 equiv). The mixture was stirred at 25° C. for 1 hour. The reaction mixture was adjusted to pH=2 with 1M HCl, diluted with H₂O (10 mL) and extracted with EtOAc (10 mL). The organic phase was washed with brine (10 mL), dried over Na₂SO₄, filtered, and concentrated under reduced pressure to afford the crude title compound (350 mg, 92% yield) as a white solid. ¹H NMR (400 MHZ, CHLOROFORM-d) δ=7.38-7.29 (m, 5H), 4.54-4.49 (m, 1H), 4.45-4.39 (m, 1H), 4.23 (qd, J=7.2, 10.8 Hz, 1H), 4.16-4.02 (m, 1H), 3.86 (dd, J=5.6, 10.8 Hz, 1H), 3.49 (dd, J=9.6, 10.8 Hz, 1H), 2.47 (dq, J=5.6, 9.2 Hz, 1H), 2.02 (dd, J=4.4, 9.2 Hz, 1H), 1.81 (dd, J=4.4, 8.4 Hz, 1H), 1.21 (t, J=7.2 Hz, 3H).

Step E. ethyl 2-((benzyloxy)methyl)-1-(dimethylcarbamoyl)cyclopropanecarboxylate: To a mixture of 2-(benzyloxymethyl)-1-ethoxycarbonyl-cyclopropanecarboxylic acid (240 mg, 1.0 equiv) and N-methylmethanamine (141 mg, 2.0 equiv, HCl salt) in DCM (3 mL) were added HATU (656 mg, 2.0 equiv) and DIEA (557 mg, 5.0 equiv). The mixture was stirred at 25° C. for 12 hours under N₂ atmosphere. The mixture was poured into ice-water (3 mL) and extracted with ethyl acetate (6 mL). The organic phase was washed with brine (3 mL), dried over anhydrous Na₂SO₄, filtered, concentrated under vacuum and purified with flash silica gel chromatography (ISCOR; 4 g SepaFlash® Silica Flash Column, Eluent of 0˜50% Ethyl acetate/Petroleum ethergradient@36 mL/min) to afford the title compound (180 mg, 68% yield) as a yellow oil. ¹H NMR (400 MHZ, CHLOROFORM-d) δ=7.38-7.29 (m, 5H), 4.52 (d, J=3.6 Hz, 2H), 4.28-4.09 (m, 2H), 3.86 (dd, J=5.6, 10.4 Hz, 1H), 3.74-3.56 (m, 1H), 3.13 (s, 3H), 2.99 (s, 3H), 2.19-2.07 (m, 1H), 1.60-1.47 (m, 2H), 1.24 (t, J=7.2 Hz, 3H).

Step F. (2-((benzyloxy)methyl)-1-((dimethylamino)methyl)cyclopropyl)methanol: To a mixture of ethyl 2-(benzyloxymethyl)-1-(dimethylcarbamoyl)cyclopropanecarboxylate (3.00 g, 1.0 equiv) in THF (30 mL) was added LAH (1.86 g, 5.0 equiv) at 0° C. The mixture was stirred at 25° C. for 1 hour under N₂ atmosphere. The mixture was cooled to 0° C. and sat. Na₂SO₄ aqueous solution (6 mL) was added to the mixture dropwise carefully. After stirring for 15 mins, the mixture was dried over anhydrous Na₂SO₄, filtered and concentrated under vacuum to afford the crude title compound (1.70 g, 69% yield) as a yellow oil.

Step G. (3R)-1-(2-((2-((benzyloxy)methyl)-1-((dimethylamino)methyl)cyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (683 mg, 1.0 equiv) and (2-((benzyloxy)methyl)-1-((dimethylamino)methyl)cyclopropyl)methanol (500 mg, 0.97 equiv) in THF (5 mL) was added NaHMDS (1 M THF solution, 1.55 mL, 1.5 equiv) at −10° C. The mixture was stirred at −10° C. for 1 hour under N₂ atmosphere. The reaction mixture was quenched with NH₄Cl aqueous solution (sat. 10 mL) at 0° C., diluted with water (10 mL) and extracted with EtOAc (20 mL). The organic layer was washed with brine (2×10 mL), dried over Na₂SO₄, filtered, concentrated under reduced pressure and purified with prep-HPLC (column: YMC Triart C18 150*25 mm*5 um; mobile phase: [water (FA)-ACN]; B %: 13%-43%, 10 min) to afford the title compound (320 mg, 42% yield) as a white solid. ¹H NMR (400 MHZ, CHLOROFORM-d) δ=9.25-9.07 (m, 1H), 8.29 (s, 1H), 7.68 (br dd, J=6.0, 8.8 Hz, 1H), 7.52 (br d, J=2.4 Hz, 1H), 7.35-7.26 (m, 5H), 6.71-6.41 (m, 2H), 5.37-5.23 (m, 2H), 4.89-4.65 (m, 1H), 4.62-4.40 (m, 5H), 3.86-3.68 (m, 1H), 3.52 (s, 4H), 3.46-3.37 (m, 1H), 3.35-3.07 (m, 2H), 2.73-2.61 (m, 6H), 2.60-2.45 (m, 2H), 2.33-2.04 (m, 2H), 1.88 (br d, J=11.6 Hz, 1H), 1.78-1.57 (m, 2H), 1.46 (br d, J=6.8 Hz, 1H), 1.32 (s, 3H), 0.87 (br dd, J=6.8, 14.0 Hz, 5H); LCMS (ESI, M+1): m/z=742.4.

Step H. (3R)-1-(2-((1-((dimethylamino)methyl)-2-(hydroxymethyl)cyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of (3R)-1-(2-((2-((benzyloxy)methyl)-1-((dimethylamino)methyl)cyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (50.0 mg, 1.0 equiv) in EtOAc (1 mL) were added Pd/C (10.0 mg, 10% purity) and Pd(OH)₂/C (10.0 mg, 20% purity) under N₂ atmosphere. The suspension was degassed under vacuum and purged with H₂ several times. The mixture was stirred at 25° C. for 2 hours under H₂ (15 Psi) atmosphere. The reaction mixture was filtered. The filtrate was concentrated under reduced pressure and purified with prep-HPLC (column: Waters Xbridge 150*25 mm*5 um; mobile phase: [water (NH₄HCO₃)-ACN]; B %: 47%-77%, 8 min) to afford the title compound (20.0 mg, 46% yield) as a white solid. ¹H NMR (400 MHZ, CHLOROFORM-d) δ=9.16 (d, J=4.4 Hz, 1H), 7.75-7.65 (m, 1H), 7.57-7.51 (m, 1H), 7.32-7.28 (m, 1H), 7.24 (td, J=2.4, 5.2 Hz, 1H), 5.43-5.24 (m, 2H), 5.14-4.99 (m, 1H), 4.64-4.35 (m, 2H), 4.30-4.14 (m, 1H), 4.08-3.92 (m, 1H), 3.60-3.39 (m, 5H), 3.38-3.20 (m, 1H), 2.98-2.84 (m, 1H), 2.62-2.45 (m, 1H), 2.29 (s, 6H), 2.16-2.08 (m, 1H), 1.98-1.60 (m, 9H), 1.36 (s, 3H), 1.25 (br d, J=7.2 Hz, 1H), 0.96-0.76 (m, 4H), 0.75-0.60 (m, 1H); LCMS (ESI, M+1): m/z=652.3.

Step I. (3R)-1-(2-((1-((dimethylamino)methyl)-2-(hydroxymethyl)cyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a solution of (3R)-1-(2-((1-((dimethylamino)methyl)-2-(hydroxymethyl)cyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (43.9 mg, 1.0 equiv) in MeOH (1 mL) was added HCl/MeOH (4 M, 0.3 mL) at 0° C. The mixture was stirred at 0° C. for 0.5 hour. The mixture was poured into NaHCO₃ aqueous solution (5 mL, sat.) and extracted with ethyl acetate (2×5 mL). The combined organic phases were washed with brine (5 mL), dried over anhydrous Na₂SO₄, filtered, concentrated under vacuum and purified with prep-HPLC (column: Phenomenex luna C18 150*25 mm*10 um; mobile phase: [water (FA)-ACN]; B %: 17%-37%, 10 min) to afford the title compound (6.50 mg, 15% yield) as a white solid. ¹H NMR (400 MHZ, METHANOL-d₄) δ=9.26 (d, J=3.2 Hz, 1H), 8.55 (br s, 1H), 7.70 (dd, J=5.6, 9.2 Hz, 1H), 7.33 (d, J=2.8 Hz, 1H), 7.32-7.23 (m, 1H), 7.08 (s, 1H), 4.77-4.63 (m, 2H), 4.63-4.52 (m, 1H), 4.34 (br t, J=12.0 Hz, 1H), 3.90 (br dd, J=5.6, 11.6 Hz, 1H), 3.72-3.55 (m, 2H), 3.54-3.41 (m, 1H), 3.23 (br s, 1H), 2.80 (br s, 7H), 2.49 (br dd, J=7.6, 14.4 Hz, 1H), 2.33-2.07 (m, 2H), 1.95-1.69 (m, 3H), 1.43 (br d, J=5.2 Hz, 1H), 1.31 (d, J=8.8 Hz, 3H), 1.01-0.75 (m, 5H); LCMS (ESI, M+1): m/z=608.2.

### Example 814

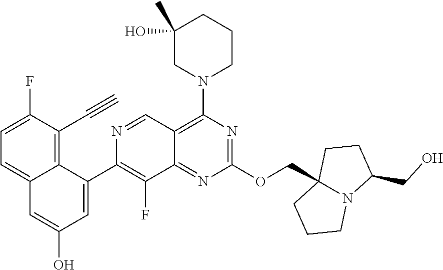 [SMILES: C#Cc1c(F)ccc2cc(O)cc(-c3ncc4c(N5CCC[C@@](C)(O)C5)nc(OC[C@]56CCCN5[C@H](CO)CC6)nc4c3F)c12]

### (R)-1-(7-(8-ethynyl-7-fluoro-3-hydroxynaphthalen-1-yl)-8-fluoro-2-(((3S,7aR)-3-(hydroxymethyl)tetrahydro-1H-pyrrolizin-7a(5H)-yl)methoxy)pyrido[4,3-d]pyrimidin-4-yl)-3-methylpiperidin-3-ol

Synthesized according to Example 809. The title compound was obtained as yellow solid (FA salt). ¹H NMR (400 MHZ, METHANOL-d4) δ=9.27-9.01 (m, 1H), 8.54 (s, 1H), 7.91-7.82 (m, 1H), 7.38-7.29 (m, 2H), 7.28-7.19 (m, 1H), 4.51 (br s, 3H), 4.48-4.40 (m, 1H), 4.39-4.26 (m, 1H), 3.73-3.60 (m, 3H), 3.50-3.35 (m, 2H), 3.28-3.10 (m, 2H), 2.39-2.27 (m, 1H), 2.25-2.15 (m, 1H), 2.14-2.00 (m, 4H), 2.00-1.88 (m, 3H), 1.87-1.68 (m, 3H), 1.28 (d, J=19.6 Hz, 3H); LCMS [ESI, M+1]: m/z=616.3.

### Example 815

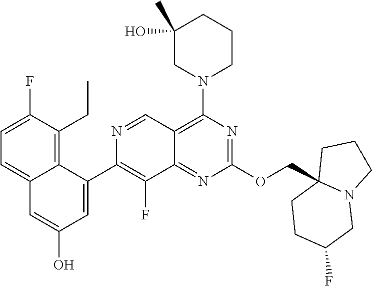 [SMILES: CCc1c(F)ccc2cc(O)cc(-c3ncc4c(N5CCC[C@@](C)(O)C5)nc(OC[C@@]56CCCN5C[C@H](F)CC6)nc4c3F)c12]

### (R)-1-(7-(8-ethyl-7-fluoro-3-hydroxynaphthalen-1-yl)-8-fluoro-2-(((6R,8aS)-6-fluorohexahydroindolizin-8a (1H)-yl)methoxy)pyrido[4,3-d]pyrimidin-4-yl)-3-methylpiperidin-3-ol

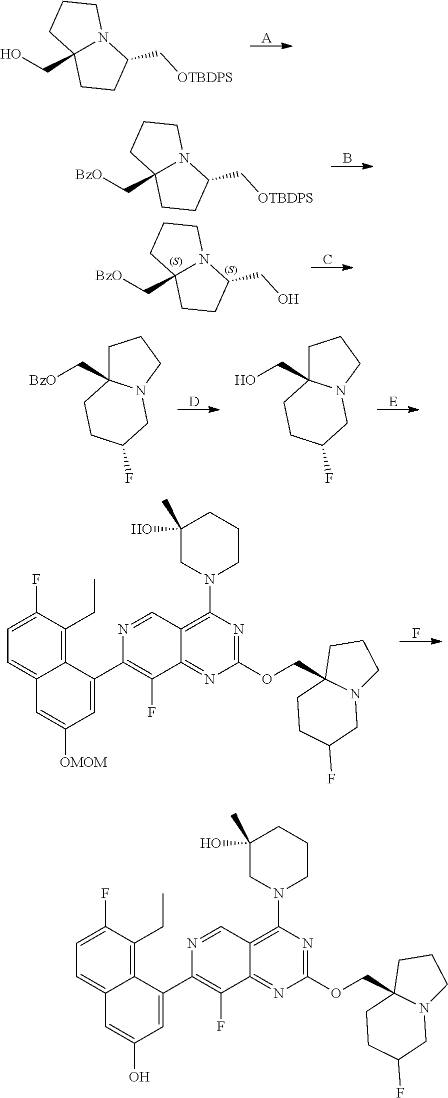

Step A. ((3S,7aS)-3-(((tert-butyldiphenylsilyl)oxy)methyl)tetrahydro-1H-pyrrolizin-7a(5H)-yl)methyl benzoate: To a solution of ((3R,7aR)-3-(((tert-butyldiphenylsilyl)oxy)methyl)tetrahydro-1H-pyrrolizin-7a(5H)-yl)methanol (2.6 g, 1 equiv) in DCM (26 mL) was added TEA (1.28 g, 1.77 mL, 2 equiv) and B₂Cl (1.34 g, 1.5 eq) at 0° C. The reaction was stirred at 25° C. for 12 hours. The mixture was washed with saturated NaHCO₃ (80 mL), extracted with DCM (3× 50 mL), dried over anhydrous Na₂SO₄, concentrated and purified by column chromatography (SiO₂, Petroleum ether/Ethyl acetate=1/0 to 1/1) to afford the tittle compound (2.9 g, 88.94% yield) s a yellow oil; ¹H NMR (400 MHZ, CHLOROFORM-d) δ=8.12-8.03 (m, 2H), 8.02-7.95 (m, 2H), 7.70-7.60 (m, 4H), 7.58-7.49 (m, 1H), 7.44-7.31 (m, 10H), 4.53-4.33 (m, 2H), 3.99 (br s, 2H), 3.76-3.64 (m, 1H), 3.47-3.33 (m, 1H), 3.20-2.98 (m, 1H), 2.20 (br dd, J=3.9, 6.2 Hz, 1H), 2.01-1.82 (m, 5H), 1.80-1.59 (m, 1H), 1.06 (s, 9H).

Step B. ((3S,7aS)-3-(hydroxymethyl)tetrahydro-1H-pyrrolizin-7a(5H)-yl)methyl benzoate: To a solution of ((3S,7aS)-3-(((tert-butyldiphenylsilyl)oxy)methyl)tetrahydro-1H-pyrrolizin-7a(5H)-yl)methyl benzoate (2.9 g, 1 equiv) in DMF (30 mL) was added CsF (8.57 g, 10 eq). The reaction was stirred at 40° C. for 12 hours. The mixture was filtered and purified by reversed phase flash chromatography [C18, 0.1% formic acid condition] to afford the tittle compound (1.5 g, 94% yield) as a yellow oil. ¹H NMR (400 MHZ, CHLOROFORM-d) δ=8.42 (s, 1H), 8.06 (dd, J=1.2, 8.4 Hz, 2H), 7.61-7.54 (m, 1H), 7.51-7.42 (m, 2H), 4.61-4.50 (m, 2H), 4.16-4.04 (m, 1H), 3.98 (dd, J=3.6, 13.2 Hz, 1H), 3.93-3.85 (m, 1H), 3.77-3.70 (m, 1H), 3.04 (dt, J=7.2, 10.7 Hz, 1H), 2.36-2.26 (m, 1H), 2.26-2.16 (m, 1H), 2.16-2.03 (m, 3H), 1.98 (dd, J=8.0, 13.2 Hz, 1H), 1.92-1.83 (m, 1H), 1.82-1.68 (m, 1H).

Step C. ((6R,8aS)-6-fluorohexahydroindolizin-8a (1H)-yl)methyl benzoate: To a solution of ((3S,7aS)-3-(hydroxymethyl)tetrahydro-1H-pyrrolizin-7a(5H)-yl)methyl benzoate (300 mg, 1 eq) in DCM (20 mL) was added DAST (526 mg, 3 equiv) at 0° C. The reaction was stirred at 20° C. for 12 hours. The reaction was set up in four batches. The reaction mixture was quenched by saturated NaHCO₃ (10 mL), extracted with DCM (2× 20 mL), dried over anhydrous Na₂SO₄, concentrated and purified by column chromatography (SiO₂, Petroleum ether/Ethyl acetate=1/0 to 1/1) to afford the tittle compound (330 mg, 35.20% yield) as a yellow oil; ¹H NMR (400 MHZ, CHLOROFORM-d) δ=8.04 (br d, J=8.0 Hz, 2H), 7.63-7.54 (m, 1H), 7.51-7.42 (m, 2H), 4.83-4.60 (m, 1H), 4.47 (d, J=11.2 Hz, 1H), 4.14 (d, J=10.8 Hz, 1H), 3.32-3.18 (m, 2H), 3.13-2.95 (m, 2H), 2.11-1.97 (m, 2H), 1.95-1.77 (m, 4H), 1.76-1.63 (m, 2H)

Step D. ((6R,8aS)-6-fluorohexahydroindolizin-8a (1H)-yl)methanol: To a solution of [(6R,8aS)-6-fluoro-2,3,5,6,7,8-hexahydro-1H-indolizin-8a-yl]methyl benzoate (200 mg, 1 equiv) in MeOH (2 mL) was added NaOMe (5 M, 144 μL, 1.0 equiv) The reaction was stirred at 20° C. for 1 hour. The mixture was quenched by water (0.5 mL), washed with petroleum ether (3×1 mL), concentrated, diluted with DCM (5 mL), dried over anhydrous sodium sulfate and concentrated to afford the tittle compound (107 mg, crude) as a yellow oil. ¹H NMR (400 MHZ, CHLOROFORM-d) δ=4.77-4.50 (m, 1H), 3.58 (d, J=10.0 Hz, 1H), 3.32 (q, J=8.4 Hz, 1H), 3.22-3.08 (m, 3H), 2.91-2.75 (m, 1H), 2.05-1.55 (m, 9H).

Step E. (R)-1-(7-(8-ethyl-7-fluoro-3-(methoxymethoxy)naphthalen-1-yl)-8-fluoro-2-(((6R,8aS)-6-fluorohexahydroindolizin-8a (1H)-yl)methoxy)pyrido[4,3-d]pyrimidin-4-yl)-3-methylpiperidin-3-ol: To a solution of [(6R,8aS)-6-fluoro-2,3,5,6,7,8-hexahydro-1H-indolizin-8a-yl]methanol (50.00 mg, 1.0 equiv) and (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (153 mg, 1.0 equiv) in THF (2 mL) was added NaHMDS (1 M, 433 μL, 1.5 eq) at 0° C. The reaction was stirred at 20° C. for 1 hour. The mixture was concentrated and purified by prep-HPLC [Phenomenex C18 75×30 mm×3 μm; A: water (FA), B: ACN, B %: 18%-48% over 7 min] to afford the tittle compound (80 mg, 39% yield) as a yellow solid. LCMS (ESI, M+1): m/z=666.3

Step F. (R)-1-(7-(8-ethyl-7-fluoro-3-hydroxynaphthalen-1-yl)-8-fluoro-2-(((6R,8aS)-6-fluorohexahydroindolizin-8a (1H)-yl)methoxy)pyrido[4,3-d]pyrimidin-4-yl)-3-methylpiperidin-3-ol: To a solution of (3R)-1-[2-[[(6R,8aS)-6-fluoro-2,3,5,6,7,8-hexahydro-1H-indolizin-8a-yl]methoxy]-7-[8-ethyl-7-fluoro-3-(methoxymethoxy)-1-naphthyl]-8-fluoro-pyrido[4,3-d]pyrimidin-4-yl]-3-methyl-piperidin-3-ol (40 mg, 1.0 equiv) in MeOH (0.5 mL) was added HCl/MeOH (2 M, 0.5 mL, 16.6 equiv). The reaction was stirred at 0° C. for 0.5 hours. The mixture was concentrated and purified by rep-HPLC [Phenomenex C18 150×25 mm×10 μm; A: water (FA), B: ACN, B %: 16%-46% over 10 min] to afford the tittle compound (8.85 mg, 23% yield) as a white solid. ¹H NMR (400 MHZ, METHANOL-d₄) δ=9.23 (s, 1H), 7.68 (dd, J=6.0, 8.8 Hz, 1H), 7.31 (d, J=2.0 Hz, 1H), 7.25 (t, J=9.2 Hz, 1H), 7.06 (s, 1H), 4.74 (br s, 1H), 4.66 (br dd, J=3.2, 11.2 Hz, 1H), 4.56 (br d, J=14.8 Hz, 2H), 4.50 (br d, J=11.2 Hz, 1H), 4.37-4.24 (m, 1H), 3.70-3.54 (m, 1H), 3.52-3.38 (m, 2H), 3.27-3.14 (m, 2H), 2.54-2.39 (m, 1H), 2.26-2.03 (m, 4H), 2.03-1.92 (m, 3H), 1.91-1.73 (m, 6H), 1.29 (d, J=9.4 Hz, 3H), 0.81 (q, J=7.1 Hz, 3H); LCMS (ESI, M+1): m/z=622.3

### Example 816

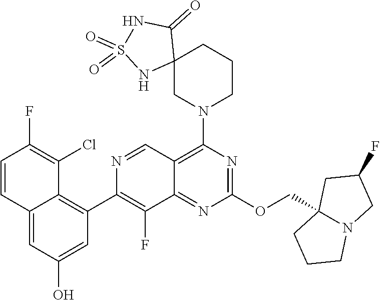 [SMILES: O=C1NS(=O)(=O)NC12CCCN(c1nc(OC[C@@]34CCCN3C[C@H](F)C4)nc3c(F)c(-c4cc(O)cc5ccc(F)c(Cl)c45)ncc13)C2]

### 7-(7-(8-chloro-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decan-4-one 2,2-dioxide

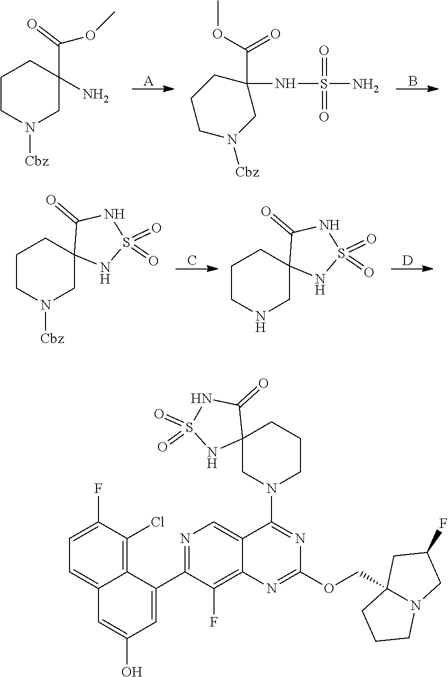

Step A. 1-benzyl 3-methyl 3-(sulfamoylamino) piperidine-1,3-dicarboxylate: To a solution of 1-benzyl 3-methyl 3-aminopiperidine-1,3-dicarboxylate (1.35 g, 1.0 equiv) in THF (10 mL) were added TEA (1.87 g, 4.0 equiv) and sulfamoyl chloride (1.07 g, 2.0 equiv) at 0° C. The reaction was stirred at 0° C. for 10 minutes and 50° C. for 24 hours. The mixture was concentrated and purified by reversed phase flash chromatography [C18, 0.1% formic acid condition] to afford the title compound (400 mg, 11% yield) as yellow oil.

Step B. benzyl 4-oxo-2-thia-1,3,7-triazaspiro[4.5]decane-7-carboxylate 2,2-dioxide: To a solution of NaOMe (349 mg, 6.0 equiv) in MeOH (2 mL) was added into a solution of 1-benzyl 3-methyl 3-(sulfamoylamino) piperidine-1,3-dicarboxylate (400 mg, 1.0 equiv) in MeOH (1.5 mL) slowly. The reaction was stirred at 68° C. for 18 hours. The mixture was concentrated and purified by reversed phase flash chromatography [C18, 0.1% formic acid condition] to afford the title compound (80 mg, 13% yield) as yellow solid; LCMS (ESI, M+23): m/z=362.0.

Step C. 2-thia-1,3,7-triazaspiro[4.5]decan-4-one 2,2-dioxide: To a mixture of Pd/C (25.0 mg, 10% purity) in MeOH (1 mL) was added benzyl 4-oxo-2-thia-1,3,7-triazaspiro[4.5]decane-7-carboxylate 2,2-dioxide (60.0 mg, 1.0 equiv) slowly under N₂ atmosphere. The reaction was degassed and purged with H₂ 3 times. The reaction was stirred under H₂ (15 Psi) at 30° C. for 1 hour. The mixture was filtered and concentrated to afford the title compound (30.0 mg, crude) as white solid.

Step D. 7-(7-(8-chloro-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decan-4-one 2,2-dioxide: To a solution of 7-(8-chloro-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) and 2-thia-1,3,7-triazaspiro[4.5]decan-4-one 2,2-dioxide (45.6 mg, crude) in DMF (0.1 mL) were added DIEA (54.8 mg, 4.0 equiv) and 4 Å molecular sieve (10 mg). The reaction was stirred at 40° C. for 12 hours. The mixture was filtered and purified by prep-HPLC [column: Phenomenex luna C18 150×25 mm×10 um; mobile phase: [water (FA)-ACN]; B %: 16%-46%, 10 min] and prep-HPLC [column: Waters Xbridge C18 150×50 mm×10 um; mobile phase: [water (NH₄HCO₃)-ACN]; B %: 17%-47%, 10 min] to afford the title compound (11.3 mg, 15% yield) as white solid; ¹H NMR (400 MHZ, METHANOL-d4) δ=9.15 (s, 1H), 7.81 (ddd, J=1.2, 5.6, 9.2 Hz, 1H), 7.43-7.35 (m, 2H), 7.26-7.19 (m, 1H), 5.68-5.47 (m, 1H), 4.81-4.55 (m, 4H), 4.54-4.39 (m, 1H), 4.16-3.95 (m, 2H), 3.93-3.80 (m, 2H), 3.49-3.38 (m, 1H), 2.62-2.41 (m, 2H), 2.87-2.40 (m, 1H), 2.37-2.22 (m, 4H), 2.15-2.01 (m, 1H), 1.93 (br dd, J=1.6, 10.8 Hz, 2H); LCMS (ESI, M+1): m/z=704.2.

### Example 817

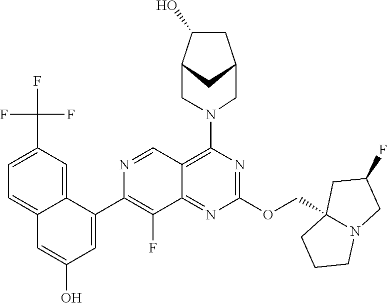 [SMILES: Oc1cc(-c2ncc3c(N4C[C@@H]5C[C@H](C4)[C@H](O)C5)nc(OC[C@@]45CCCN4C[C@H](F)C5)nc3c2F)c2cc(C(F)(F)F)ccc2c1]

### (1R,5R,6R)-3-(8-fluoro-2-(((2R,7aS)-2-fluorotetrahydro-1H-pyrrolizin-7a(5H)-yl)methoxy)-7-(3-hydroxy-7-(trifluoromethyl)naphthalen-1-yl)pyrido[4,3-d]pyrimidin-4-yl)-3-azabicyclo[3.2.1]octan-6-ol

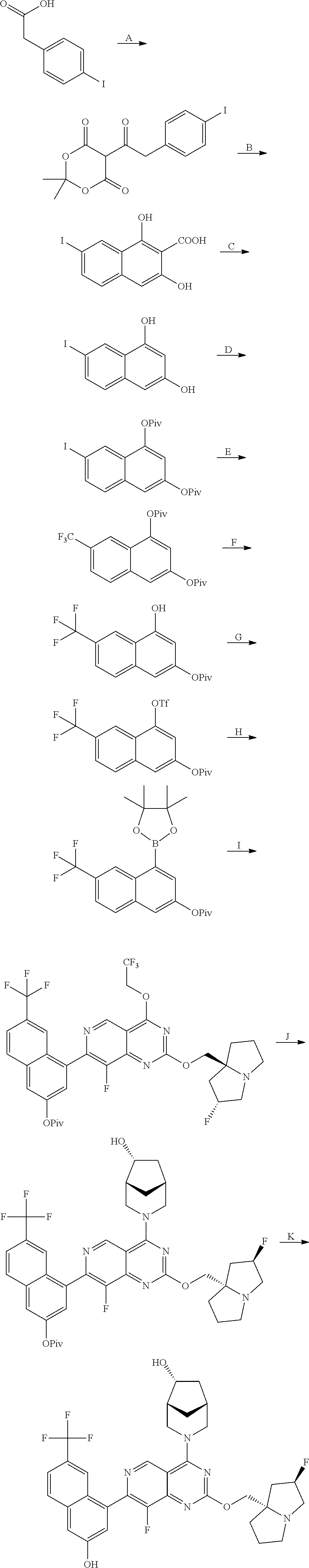

Step A. 5-(2-(4-iodophenyl) acetyl)-2,2-dimethyl-1,3-dioxane-4,6-dione: To a solution of 2-(4-iodophenyl) acetic acid (30.0 g, 1.0 equiv) and 2,2-dimethyl-1,3-dioxane-4,6-dione (18.1 g, 1.1 equiv) in acetonitrile (150 mL) were added DMAP (1.26 g, 0.09 equiv) and DIEA (31.1 g, 2.1 equiv) below 30° C. To the mixture was added pivaloyl chloride (15.2 g, 1.1 equiv) dropwise below 40° C. The reaction was stirred at 45° C. for 3 hours. The mixture was cooled to 0° C. and adjusted the pH to 5 by adding 4 M HCl (500 mL) below 15° C. The mixture was stirred at 0° C. for 1 hour. The mixture was diluted with water (1 L) and the pH of the mixture was adjusted to 2 with 4N HCl. The mixture was filtered. The filter cake was washed with water until the pH of filtrate was 5˜6. The filter cake was the title compound (40.0 g, 89% yield) as white solid; LCMS (ESI, M−1): m/z=386.9.

Step B. 1,3-dihydroxy-7-iodo-2-naphthoic acid: 5-(2-(4-iodophenyl) acetyl)-2,2-dimethyl-1,3-dioxane-4,6-dione (40.0 g, 1.0 equiv) was added into CF₃SO₃H (136 g, 8.8 equiv) in portions below 30° C. The reaction was stirred at 20° C. for 2 hours. The mixture was poured into ice water (500 mL) slowly and filtered. The filter cake was washed with water until the pH of the filtrate was 3˜4. The filter cake was the title compound (20.0 g, 57% yield) as yellow solid; LCMS (ESI, M−1): m/z=328.8.

Step C. 7-iodonaphthalene-1,3-diol: A mixture of 1,3-dihydroxy-7-iodo-2-naphthoic acid (30.0 g, 1.0 equiv) in acetonitrile (600 mL) and water (450 mL) was stirred at 90° C. for 12 hours. The mixture was diluted with water (200 mL) and extracted with ethyl acetate (2× 500 mL). The combined organic layers were washed with brine (500 mL), dried over anhydrous sodium sulfate, concentrated, and purified by column chromatography [SiO₂, Petroleum ether: Ethyl acetate=5/1 to 1/1] to afford the title compound (10.0 g, 46% yield) as brown solid.

Step D. 7-iodonaphthalene-1,3-diyl bis(2,2-dimethylpropanoate); To a solution of 7-iodonaphthalene-1,3-diol (4.00 g, 1.0 equiv), DIEA (9.04 g. 12.2 mL, 5.0 equiv) and DMAP (170 mg, 0.1 equiv) in THF (40 mL) was added pivaloyl chloride (5.90 g, 3.5 equiv) at 0° C. The reaction was stirred at 0° C. for 12 hours. The mixture was diluted with water (100 mL) and extracted with ethyl acetate (3×100 mL). The combined organic layers were washed with brine (150 mL), dried over anhydrous sodium sulfate, concentrated, and purified by column chromatography [SiO₂, Petroleum ether:Ethyl acetate=1/0] to afford the title compound (4.00 g, 59% yield) as white solid.

Step E. 7-(trifluoromethyl)naphthalene-1,3-diyl bis(2,2-dimethylpropanoate): To a mixture of 7-iodonaphthalene-1,3-diyl bis(2,2-dimethylpropanoate) (6.00 g, 1.0 equiv) and CuI (25.1 g, 10 equiv) in DMAC (90 mL) was added methyl 2,2-difluoro-2-(fluorosulfonyl)acetate (53.3 g, 21 equiv) at 25° C. The reaction was stirred at 90° C. for 12 hours. The mixture was diluted with water (100 mL) and extracted with ethyl acetate (3×100 mL). The combined organic layers were washed with brine (100 mL), dried over anhydrous sodium sulfate, concentrated, and purified by column chromatography [SiO₂, Petroleum ether:Ethyl acetate=1/0] to afford the title compound (2.00 g, 38% yield) as white solid.

Step F. 4-hydroxy-6-(trifluoromethyl)naphthalen-2-yl pivalate: To a solution of 7-(trifluoromethyl)naphthalene-1,3-diyl bis(2,2-dimethylpropanoate) (1.00 g, 1.0 equiv) in THF (20 mL) and H₂O (5 mL) was added LiOH·H₂O (233 mg, 2.2 equiv). The reaction was stirred at 0° C. for 1.5 hours. The mixture was diluted with water (10 mL) and adjusted the pH to 2 with 2 M HCl. The mixture was extracted with ethyl acetate (20 mL). The combined organic layers were concentrated and purified by column chromatography [SiO₂, Petroleum ether:Ethyl acetate=1/0 to 4/1] to afford the title compound (260 mg. 30% yield) as white solid; ¹H NMR (400 MHZ, DMSO-d₆) δ=11.57-10.42 (m, 1H), 8.42 (s, 1H), 8.05 (d, J=8.4 Hz, 1H), 7.74 (dd, J=1.6, 8.4 Hz, 1H), 7.25 (d, J=1.6 Hz, 1H), 6.72 (d, J=2.0 Hz, 1H), 1.34 (s, 9H).

Step G. 6-(trifluoromethyl)-4-(((trifluoromethyl) sulfonyl)oxy)naphthalen-2-yl pivalate: To a solution of 4-hydroxy-6-(trifluoromethyl)naphthalen-2-yl pivalate (330 mg, 1.0 equiv) and DIEA (273 mg, 2.0 equiv) in DCM (5 mL) was added Tf₂O (358 mg, 1.2 equiv) at −40° C. The reaction was stirred at −40° C. for 0.5 hours. The mixture was concentrated and purified by column chromatography [SiO₂, Petroleum ether:Ethyl acetate=100/1 to 16/1] to afford the title compound (330 mg, 66% yield) as brown solid, ¹H NMR (400 MHZ, CHLOROFORM-d) δ=8.36 (s, 1H), 8.01 (d, J=8.4 Hz, 1H), 7.79 (dd, J=1.6, 8.8 Hz, 1H), 7.72 (d, J=1.6 Hz, 1H), 7.41 (d, J=2.0 Hz, 1H), 1.46-1.40 (m, 9H).

Step H. 4-(4,4,5,5-tetramethyl-1,3,2-dioxaborolan-2-yl)-6-(trifluoromethyl)naphthalen-2-yl pivalate: To a solution of 6-(trifluoromethyl)-4-(((trifluoromethyl) sulfonyl)oxy)naphthalen-2-yl pivalate (330 mg, 1.0 equiv), 4,4,5,5-tetramethyl-1,3,2-dioxaborolane (380 mg, 4.0 equiv) and TEA (301 mg, 4.0 equiv) in ACN (12 mL) was added Pd(dppf) C12 (54.3 mg, 0.1 equiv). The reaction was degassed and purged with nitrogen 3 times. The reaction was stirred at 80° C. for 2 hours. The mixture was quenched with MeOH (0.5 mL). The mixture was concentrated and purified by column chromatography [SiO₂, Petroleum ether: Ethyl acetate=100/1 to 6/1] to afford the title compound (170 mg, 25% yield) as white solid; ¹H NMR (400 MHZ, CHLOROFORM-d) δ=9.17 (s, 1H), 7.97 (s, 1H), 7.88-7.83 (m, 1H), 7.71-7.67 (m, 1H), 7.66-7.61 (m, 1H), 1.43 (d, J=6.4 Hz, 21H).

Step I. 4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-6-(trifluoromethyl)naphthalen-2-yl pivalate: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (156 mg, 1.0 equiv), 4-(4,4,5,5-tetramethyl-1,3,2-dioxaborolan-2-yl)-6-(trifluoromethyl)naphthalen-2-yl pivalate (150 mg, 1.0 equiv) and Cs₂CO₃ (1.5 M in water, 3.0 equiv) in methoxycyclopentane (2.5 mL) was added CataCXium A Pd G3 (25.9 mg, 0.1 equiv). The reaction was degassed and purged with nitrogen 3 times. The reaction was stirred at 90° C. for 1 hour. The mixture was diluted with water (5 mL) and extracted with ethyl acetate (2×15 mL). The combined organic layer was concentrated to afford the title compound (400 mg, crude) as white solid; LCMS (ESI, M+1): m/z=699.3.

Step J. 4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)-6-(trifluoromethyl)naphthalen-2-yl pivalate: To a mixture of 4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-6-(trifluoromethyl)naphthalen-2-yl pivalate (360 mg, 1.0 equiv), (1R,5R,6R)-3-azabicyclo[3.2.1]octan-6-ol (131 mg, 2.0 equiv) and 4 Å molecular sieve (100 mg) in DMF (1.5 mL) was added DIEA (200 mg, 3.0 equiv). The reaction was stirred at 40° C. for 1 hour. The mixture was filtered. The filtrate was diluted with water (10 mL) and extracted with ethyl acetate (2×15 mL). The combined organic layers were concentrated to give the title compound (600 mg, crude) as yellow oil.

Step K. (1R,5R,6R)-3-(8-fluoro-2-(((2R,7aS)-2-fluorotetrahydro-1H-pyrrolizin-7a(5H)-yl)methoxy)-7-(3-hydroxy-7-(trifluoromethyl)naphthalen-1-yl)pyrido[4,3-d]pyrimidin-4-yl)-3-azabicyclo[3.2.1]octan-6-ol: To a solution of 4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)pyrido[4,3-d]pyrimidin-7-yl)-6-(trifluoromethyl)naphthalen-2-yl pivalate (600 mg, 1.0 equiv) in THF (8 mL) was added LiOH·H₂O (3 M in water, 2.0 equiv). The reaction was stirred at 15° C. for 0.5 hours. The mixture was diluted with water (10 mL) and extracted with ethyl acetate (2×15 mL). The combined organic layers were concentrated and purified by reversed phase flash chromatography [C18, 0.1% formic acid condition] and prep-HPLC [column: Unisil 3-100 C18 Ultra 150×50 mm×3 μm; A: water (FA), B: ACN, B %: 19%-49% over 7 min] and prep-HPLC [column: Waters Xbridge BEH C18 150×25 mm×5 μm; A: water (NH₄HCO₃), B: ACN, B %: 52%-82% over 10 min] to afford the title compound (26.5 mg, 13% yield over three steps) as white solid; ¹H NMR (400 MHZ, METHANOL-d₄) δ=9.33 (s, 1H), 7.94 (d, J=8.8 Hz, 1H), 7.88 (s, 1H), 7.61 (dd, J=1.6, 8.4 Hz, 1H), 7.38 (s, 2H), 5.46-5.21 (m, 1H), 4.94 (br d, J=13.2 Hz, 1H), 4.79 (br d, J=13.2 Hz, 1H), 4.44-4.18 (m, 3H), 3.78 (br d, J=11.6 Hz, 1H), 3.54-3.32 (m, 2H), 3.30-3.20 (m, 2H), 3.10-2.98 (m, 1H), 2.46-2.10 (m, 6H), 2.07-1.97 (m, 2H), 1.96-1.85 (m, 2H), 1.84-1.77 (m, 1H), 1.40 (td, J=3.2, 14.0 Hz, 1H); LCMS (ESI, M+1): m/z=642.3.

### Example 818

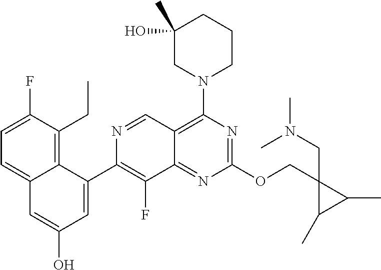 [SMILES: CCc1c(F)ccc2cc(O)cc(-c3ncc4c(N5CCC[C@@](C)(O)C5)nc(OCC5(CN(C)C)C(C)C5C)nc4c3F)c12]

### (3R)-1-(2-((1-((dimethylamino)methyl)-2,3-dimethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol

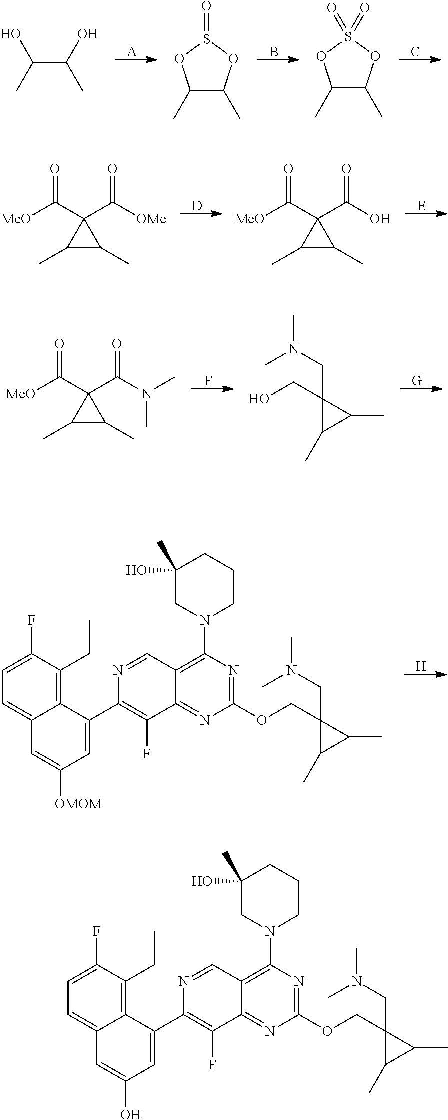

Step A. 4,5-dimethyl-1,3,2-dioxathiolane 2-oxide: To a solution of butane-2,3-diol (1.00 g, 1.0 equiv) in DCM (10 mL) was added SOCl₂ (1.72 g, 1.3 equiv). The reaction was degassed, purged with N₂ for 3 times and stirred at 40° C. for 2 hours. The mixture was poured into ice-water (10 mL) and stirred for 30 minutes, and then extracted with DCM (2×30 mL). The combined organic layers were washed with brine (30 mL), dried over sodium sulfate and concentrated to afford the title compound (1.50 g, crude) as a yellow oil; ¹H NMR (400 MHZ, CHLOROFORM-d) δ=5.16-4.95 (m, 2H), 1.33-1.28 (m, 6H).

Step B. 4,5-dimethyl-1,3,2-dioxathiolane 2,2-dioxide: To a solution of 4,5-dimethyl-1,3,2-dioxathiolane 2-oxide (10.0 g, 1.0 equiv) in a mixed solvent of DCM (30 mL) and ACN (30 mL) was added a solution of RuCl₃ (15.2 mg, 0.001 equiv) in H₂O (60 mL) dropwise, followed by addition of NaIO₄ (17.3 g, 1.1 equiv) in portions at 0° C. The reaction was stirred at 25° C. for 12 hours. The mixture was poured into ice-water (30 mL) and stirred for 30 min, and then extracted with DCM (2×30 mL). The combined organic layers were washed with brine (2×4 mL), dried over sodium sulfate, concentrated under vacuum and purified by flash silica gel chromatography (ISCO®; 12 g SepaFlash® Silica Flash Column, Eluent of 0˜ 50% Ethylacetate/Petroleum ethergradient@48 mL/min) to afford the title compound (10.5 g, 94% yield) as a yellow oil. ¹H NMR (400 MHZ, CHLOROFORM-d) δ=5.26-5.00 (m, 2H), 1.54-1.41 (m, 6H).

Step C. dimethyl 2,3-dimethylcyclopropane-1,1-dicarboxylate; To a solution of dimethyl propanedioate (6.77 g, 1.2 equiv) in DMF (60 mL) was added NaH (4.27 g, 60% purity, 2.5 equiv) at 0° C. The reaction was stirred at 0° C. for 0.5 hour. 4,5-Dimethyl-1,3,2-dioxathiolane 2,2-dioxide (6.50 g, 1.0 equiv) was added at 0° C. The reaction was stirred at 80° C. for 11.5 hours under N₂ atmosphere. The mixture was poured into ice-water (300 mL) and stirred for 30 minutes, and then extracted with ethyl acetate (2×300 mL). The combined organic layers were washed with brine (2×200 mL), dried over sodium sulfate, concentrated under vacuum and purified by flash silica gel chromatography (ISCO®; 80 g SepaFlash® Silica Flash Column, Eluent of 0˜ 20% Ethylacetate/Petroleum ethergradient@150 mL/min) to afford the title compound (3.60 g, 45% yield) as a yellow oil. ¹H NMR (400 MHZ, CHLOROFORM-d) δ=3.75 (s, 3H), 3.71 (s, 3H), 1.85-1.77 (m, 2H), 1.15-1.11 (m, 6H).

Step D. 1-(methoxycarbonyl)-2,3-dimethylcyclopropanecarboxylic acid: To a solution of dimethyl 2,3-dimethylcyclopropane-1,1-dicarboxylate (1.60 g, 1.0 equiv) in MeOH (16 mL) was added NaOH aqueous solution (2 M, 5.16 mL, 1.2 equiv). The reaction was stirred at 25° C. for 1 hour. The mixture was acidified with 1M HCl until pH=2, diluted with H₂O (50 mL) and extracted with ethyl acetate (30 mL). The organic phase was washed with brine (50 mL), dried over sodium sulfate, filtered and concentrated under vacuum to afford the title compound (1.48 g, crude) as a white solid. ¹H NMR (400 MHZ, CHLOROFORM-d) δ=3.87-3.80 (m, 3H), 2.21-2.10 (m, 2H), 1.24-1.20 (m, 6H).

Step E. methyl 1-(dimethylcarbamoyl)-2,3-dimethylcyclopropanecarboxylate; A mixture of 1-(methoxycarbonyl)-2,3-dimethylcyclopropanecarboxylic acid (925 mg, 1.0 equiv), N-methylmethanamine (876 mg, 2.0 equiv, HCl salt), HATU (4.08 g, 2.0 equiv) and DIEA (3.47 g, 5.0 equiv) in DCM (10 mL) was degassed and purged with N₂ for 3 times. The reaction was stirred at 25° C. for 12 hours. The mixture was poured into ice-water (30 mL) and stirred for 5 minutes, and then extracted with ethyl acetate (60 mL). The organic phase was washed with brine (30 mL), dried over sodium sulfate, concentrated under vacuum and purified by flash silica gel chromatography (ISCO®; 12 g SepaFlash® Silica Flash Column, Eluent of 0˜50% Ethylacetate/Petroleum ethergradient@36 mL/min) to afford the title compound (800 mg, 75% yield) as a yellow oil. ¹H NMR (400 MHZ, DMSO-d₆) δ=3.67-3.57 (m, 3H), 2.98-2.87 (m, 3H), 2.86-2.76 (m, 3H), 1.73-1.61 (m, 2H), 1.22-1.12 (m, 6H).

Step F. (1-((dimethylamino)methyl)-2,3-dimethylcyclopropyl)methanol: To a mixture of methyl 1-(dimethylcarbamoyl)-2,3-dimethylcyclopropanecarboxylate (1.00 g, 1.0 equiv) in THF (10 mL) was added LiAlH₄ (890 mg, 5.0 equiv) in portions at 0° C. The reaction was stirred at 25° C. for 1 hour. The mixture was quenched with saturated Na₂SO₄ aqueous solution (6 mL), filtered and the filtrate was dried over sodium sulfate, concentrated to afford the title compound (737 mg, crude) as a yellow oil.

Step G. (3R)-1-(2-((1-((dimethylamino)methyl)-2,3-dimethylcyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (433 mg, 1.0 and (1-((dimethylamino)methyl)-2,3-dimethylcyclopropyl)methanol (200 mg, 0.97 equiv) in THF (4 mL) was added NaHMDS (1 M, 983 μL, 1.5 equiv) dropwise at 0° C. The reaction was stirred at −10° C. for 1 hour under N₂ atmosphere. The mixture was quenched by addition of saturated NH₄Cl aqueous solution (10 mL) at 0° C., diluted with water (10 mL) and extracted with ethyl acetate (20 mL). The combined organic layers were washed with brine (2×10 mL), dried over sodium sulfate, concentrated and purified by prep-HPLC [column: Phenomenex luna C18 150×25 mm×10 μm; A: water (FA), B: ACN, B %: 22%-52% over 10 min] to afford the title compound (152 mg, 36% yield) as a white solid; LCMS (ESI, M+1): m/z=650.3.

Step H. (3R)-1-(2-((1-((dimethylamino)methyl)-2,3-dimethylcyclopropyl)methoxy)-7-(8-ethyl-7-fluoro-3-hydroxynaphthalen-1-yl)-8-fluoropyrido[4,3-d]pyrimidin-4-yl)-3-methylpiperidin-3-ol: A mixture of (3R)-1-(2-((1-((dimethylamino)methyl)-2,3-dimethylcyclopropyl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (120 mg, 1.0 equiv) in HCl/MeOH (4 M, 1 mL) was degassed and purged with N₂ for 3 times. The reaction was stirred at −10° C. for 1 hour under N₂ atmosphere. The mixture was quenched by addition of saturated NH₄Cl aqueous solution (10 mL) at 0° C., diluted with water (10 mL) and extracted with ethyl acetate (20 mL). The combined organic layers were washed with brine (2×10 mL), dried over sodium sulfate, concentrated and purified by prep-HPLC [column: Phenomenex luna C18 150×25 mm×10 μm; A: water (FA), B: ACN, B %: 16%-46% over 10 min] to afford the title compound (54.4 mg, 59% yield) as a white solid. ¹H NMR (400 MHZ, METHANOL-d₄) δ=9.25 (d, J=2.8 Hz, 1H), 8.62-8.52 (m, 1H), 7.70 (dd, J=6.0, 9.2 Hz, 1H), 7.33 (d, J=2.8 Hz, 1H), 7.28 (t, J=9.2 Hz, 1H), 7.08 (s, 1H), 4.69-4.60 (m, 3H), 4.32 (br t, J=12.8 Hz, 1H), 3.72-3.59 (m, 1H), 3.56-3.44 (m, 1H), 2.99-2.82 (m, 2H), 2.79-2.59 (m, 6H), 2.56-2.40 (m, 1H), 2.31-2.11 (m, 2H), 1.97-1.73 (m, 3H), 1.31 (d, J=9.2 Hz, 3H), 1.18 (br d, J=5.2 Hz, 6H), 1.13-1.00 (m, 2H), 0.83 (q, J=7.2 Hz, 3H); ¹⁹F NMR (376 MHZ, METHANOL-d₄) δ=−121.12 (br s, 1F), −139.82 (br s, 1F); LCMS (ESI, M+1): m/z=606.4.

### Example 819

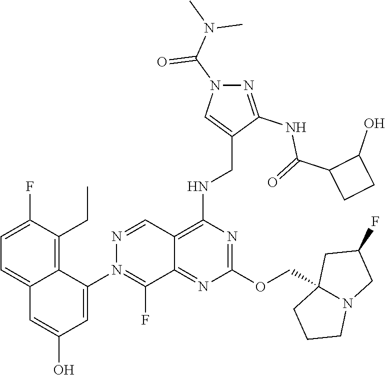

### 4-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-3-(2-hydroxycyclobutane-1-carboxamido)-N,N-dimethyl-1H-pyrazole-1-carboxamide

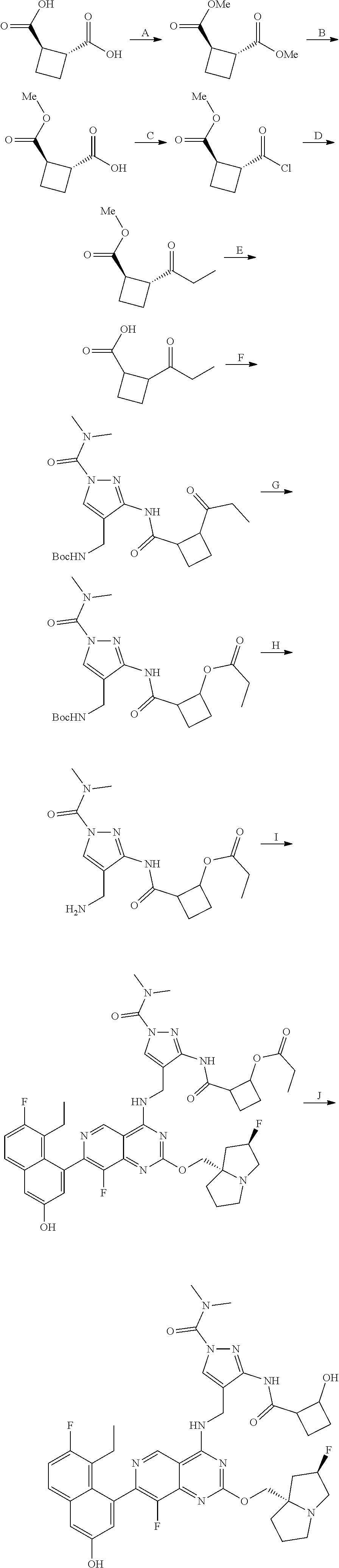

Step A. dimethyl (1R,2R)-cyclobutane-1,2-dicarboxylate: To a solution of (1R,2R)-cyclobutane-1,2-dicarboxylic acid (500 mg, 1.0 equiv) in MeOH (5 mL) was added sulphuric acid (460 mg, 1.4 equiv). The reaction was stirred at 40° C. for 16 hours. The mixture was concentrated under reduced pressure, diluted with water (50 mL) and extracted with ethyl acetate (50 mL×3). The combined organic layers were washed with saturated sodium bicarbonate solution (50 mL), dried over anhydrous sodium sulfate, filtered and concentrated to afford the title compound (515 mg, 86% yield) as yellow oil; ¹H NMR (400 MHZ, CDCl₃) δ=3.69 (s, 6H), 3.33-3.47 (m, 2H), 2.13-2.20 (m, 4H).

Step B. (1R,2R)-2-(methoxycarbonyl)cyclobutane-1-carboxylic acid: To a solution of dimethyl (1R,2R)-cyclobutane-1,2-dicarboxylate (500 mg, 1.0 equiv) in dioxane (8 mL) and H₂O (8 mL) was added NaOH (1 M, 2.90 mL, 1.0 equiv). The reaction was stirred at 25° C. for 3 hours. The mixture was adjusted to pH=2-3 with 1M HCl (5 mL) and extracted with ethyl acetate (40 mL×3). The combined organic layers were dried over Na₂SO₄, filtered, concentrated under reduced pressure and purified by column chromatography (SiO₂, Petroleum ether/Ethyl acetate=5/1 to 1/1) to afford the title compound (360 mg, 78% yield) as yellow oil; ¹H NMR (400 MHz, CDCl₃) δ=3.66-3.78 (m, 3H), 3.39-3.52 (m, 2H), 2.17-2.30 (m, 4H).

Step C. methyl (1R,2R)-2-(chlorocarbonyl)cyclobutane-1-carboxylate: To a solution of (1R,2R)-2-(methoxycarbonyl)cyclobutane-1-carboxylic acid (350 mg, 1.0 equiv) and oxalyl chloride (308 mg, 1.1 equiv) in DCM (6 mL) was added DMF (51.7 mg, 0.32 equiv) at 0° C. The reaction was stirred at 25° C. for 2 hours. The mixture was concentrated under reduced pressure to afford the title compound (350 mg, 89.55% yield) as yellow oil.

Step D. methyl (1R,2R)-2-propionylcyclobutane-1-carboxylate: A mixture of methyl (1R,2R)-2-(chlorocarbonyl)cyclobutane-1-carboxylate (350 mg, 1.0 equiv) and 1,1′-Bis(diphenylphosphino)ferrocenepalladium(II) dichloride (72.51 mg, 0.05 eq) in toluene (5 mL) was degassed and purged with N₂ for 3 times. ZnEt₂ (1 M, 2.38 mL, 1.2 equiv) was added to above mixture at 0° C. The reaction was stirred at 25° C. for 3 hours under N₂ atmosphere. The mixture was quenched with water (5 mL) at 0° C. and extracted with ethyl acetate (5 mL×3). The combined organic layers were dried over Na₂SO₄, filtered, concentrated under reduced pressure and purified by column chromatography (SiO₂, Petroleum ether/Ethyl acetate=5/1) to afford the title compound (150 mg, 44% yield) as a yellow oil; ¹H NMR (400 MHZ, CDCl₃) δ=3.67-3.73 (m, 3H), 3.33-3.57 (m, 2H), 2.37-2.47 (m, 2H), 2.05-2.20 (m, 4H), 1.03-1.08 (m, 3H).

Step E. 2-propionylcyclobutane-1-carboxylic acid: To a solution of methyl (1R,2R)-2-propionylcyclobutane-1-carboxylate (3.00 g, 1.0 equiv) in THF (20 mL) and H₂O (10 mL) was added KOH (2.97 g, 3.0 equiv). The reaction was stirred at 25° C. for 3 hours. The mixture was diluted with water (30 mL) and extracted with ethyl acetate (40 mL×3). The combined organic layers were dried over Na₂SO₄, filtered, concentrated under reduced pressure and purified by column chromatography (SiO₂, Petroleum ether/Ethyl acetate=1/1) to afford the title compound (2.00 g, 73% yield) as a yellow oil; ¹H NMR (400 MHZ, DMSO-d₆) δ=12.08-12.14 (m, 1H), 3.38-3.49 (m, 1H), 3.13-3.23 (m, 1H), 2.41 (dd, J=7.2, 1.2 Hz, 2H), 1.96-2.05 (m, 4H), 0.92 (t, J=7.2 Hz, 3H).

Step F. tert-butyl((1-(dimethylcarbamoyl)-3-(2-propionylcyclobutane-1-carboxamido)-1H-pyrazol-4-yl)methyl)carbamate: To a solution of tert-butyl((3-amino-1-(dimethylcarbamoyl)-1H-pyrazol-4-yl)methyl)carbamate (200 mg, 1.0 equiv) and (2-propionylcyclobutane-1-carboxylic acid (551 mg, 5.0 equiv) in DCM (10 mL) were added DIEA (365 mg, 4.0 equiv) and HATU (537 mg, 2.0 equiv). The reaction was stirred at 25° C. for 3 hours. The mixture was diluted with water (30 mL) and extracted with ethyl acetate (40 mL×3). The combined organic layers were dried over Na₂SO₄, filtered and concentrated under reduced pressure to afford the title compound (200 mg, 67% yield) as yellow oil; LCMS (ESI, M+1): m/z=422.4.

Step G. 2-((4-(((tert-butoxycarbonyl)amino)methyl)-1-(dimethylcarbamoyl)-1H-pyrazol-3-yl)carbamoyl)cyclobutyl propionate: To a solution of tert-butyl((1-(dimethylcarbamoyl)-3-(2-propionylcyclobutane-1-carboxamido)-1H-pyrazol-4-yl)methyl)carbamate (200 mg, 1.0 equiv) and trifluoroacetic anhydride (1.49 g, 15 equiv) in DCM (20 mL) was added H₂O₂ (807 mg, 30% purity, 15 equiv) at 0° C. The reaction was stirred at 25° C. for 3 hours. The mixture was quenched with aqueous sodium sulfite solution (60 mL) at 0° C., adjusted to pH=8 with saturated sodium bicarbonate solution and extracted with ethyl acetate (60 mL×3). The combined organic layers were dried over Na₂SO₄, filtered and concentrated under reduced pressure to afford the title compound (200 mg, 96% yield) as yellow solid; LCMS (ESI, M+1): m/z=438.2.

Step H. 2-((4-(aminomethyl)-1-(dimethylcarbamoyl)-1H-pyrazol-3-yl)carbamoyl)cyclobutyl propionate: To a solution of 2-((4-(((tert-butoxycarbonyl)amino)methyl)-1-(dimethylcarbamoyl)-1H-pyrazol-3-yl)carbamoyl)cyclobutyl propionate (150 mg, 1.0 equiv) in dioxane (3 mL) was added HCl/dioxane (4 M, 4.2 equiv) at 0° C. The reaction was stirred at 25° C. for 1 hour. The mixture was concentrated under reduced pressure to afford the title compound (100 mg, 86% yield) as yellow oil; LCMS (ESI, M+1): m/z=338.0

Step I. 2-((1-(dimethylcarbamoyl)-4-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1H-pyrazol-3-yl)carbamoyl)cyclobutyl propionate: To a mixture of 2-((4-(aminomethyl)-1-(dimethylcarbamoyl)-1H-pyrazol-3-yl)carbamoyl)cyclobutyl propionate (30.0 mg, 1.0 equiv), 5-ethyl-6-fluoro-4-(8-fluoro-2-(((2R,7aS)-2-fluorotetrahydro-1H-pyrrolizin-7a(5H)-yl)methoxy)-4-(trifluoromethoxy)pyrido[4,3-d]pyrimidin-7-yl)naphthalen-2-ol (52.7 mg, 1.0 equiv) in MeCN (1 mL) and DMF (1 mL) were added and K₃PO₄ (189 mg, 10 eq) and 4 Å molecular sieves (300 mg). The reaction was stirred at 60° C. for 1 hour. The mixture was filtered and purified by prep-HPLC [Waters xbridge 150×25 mm 10 um; mobile phase: water (NH₄HCO₃)-ACN; B %: 31%-61%, 8 min] to afford the title compound (20 mg, 27% yield) as yellow solid; LCMS (ESI, M+1): m/z=830.5.

Step J. 4-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-3-(2-hydroxycyclobutane-1-carboxamido)-N,N-dimethyl-1H-pyrazole-1-carboxamide: To a solution of 2-((1-(dimethylcarbamoyl)-4-(((7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)amino)methyl)-1H-pyrazol-3-yl)carbamoyl)cyclobutyl propionate (15.0 mg, 1.0 equiv) in MeOH (1 mL) was added K₂CO₃ (0.1 M, 0.7 mL, 4.0 equiv). The reaction was stirred at 25° C. for 0.5 hours. The mixture was quenched with HCOOH (0.5 M, 0.5 mL, 10 equiv) at 25° C., filtered and purified by prep-HPLC [Phenomenex luna C18 150×25 mm×10 um; mobile phase: water (FA)-ACN; B %: 12%-42%, 8 min] to afford the title compound (1.97 mg, 12% yield, HCOOH) as white solid; ¹H NMR (400 MHZ, METHANOL-d₄) δ=9.12-9.17 (m, 1H), 8.46-8.58 (m, 1H), 8.16-8.27 (m, 1H), 7.63-7.72 (m, 1H), 7.28-7.33 (m, 1H), 7.20-7.28 (m, 1H), 6.99-7.06 (m, 1H), 5.45-5.54 (m, 1H), 5.26-5.44 (m, 1H), 4.65-4.76 (m, 2H), 4.30-4.49 (m, 2H), 3.50-3.68 (m, 1H), 3.35 (br s, 4H), 3.13 (br s, 8H), 2.41-2.53 (m, 3H), 2.27-2.34 (m, 1H), 2.20-2.25 (m, 1H), 2.08 (br d, J=6.4 Hz, 3H), 1.96 (br dd, J=10.8, 6.4 Hz, 1H), 1.73-1.84 (m, 1H), 0.73-0.82 (m, 3H); LCMS (ESI, M+1): m/z=774.8.

### Example 820

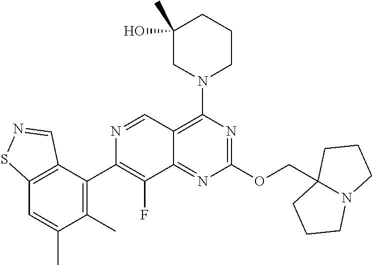 [SMILES: Cc1cc2sncc2c(-c2ncc3c(N4CCC[C@@](C)(O)C4)nc(OCC45CCCN4CCC5)nc3c2F)c1C]

### (3R)-1-(7-(5,6-dimethylbenzo[d]isothiazol-4-yl)-8-fluoro-2-((tetrahydro-1H-pyrrolizin-7a(5H)-yl)methoxy)pyrido[4,3-d]pyrimidin-4-yl)-3-methylpiperidin-3-ol

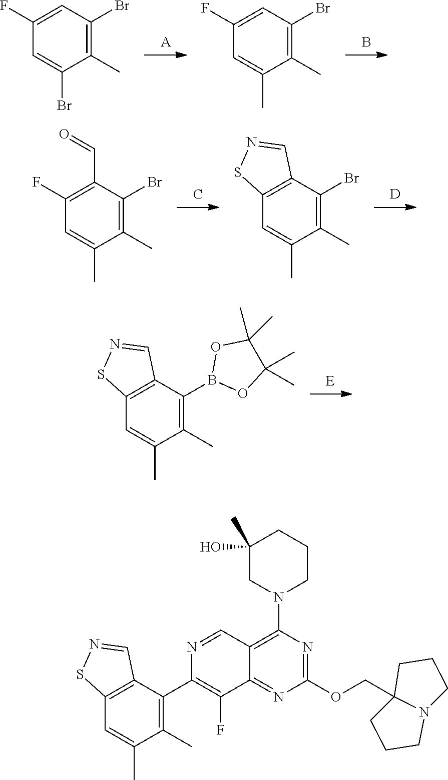

Step A. 1-bromo-5-fluoro-2,3-dimethylbenzene: To a mixture of 1,3-dibromo-5-fluoro-2-methylbenzene (3.5 g, 1 equiv), potassium trifluoro(methyl) borate (2.07 g, 1.3 equiv) in dioxane (50 mL) and H₂O (10 mL) were added 1,1′-Bis(diphenylphosphino)ferrocene-palladium(II) dichloride (956 mg, 0.1 equiv) and K₂CO₃ (3.61 g, 2 equiv). The reaction was degassed and purged with nitrogen 3 times. The reaction was stirred at 90° C. for 12 hours. The mixture diluted with petroleum ether (100 mL) and washed with brine (2×100 mL). The organic layer was dried over anhydrous sodium sulfate, concentrated and purified with column chromatography (SiO₂, petroleum ether/ethyl acetate=1/0) to afford the title compound (1.8 g, crude) as light yellow oil.

Step B. 2-bromo-6-fluoro-3,4-dimethylbenzaldehyde: To a solution of 1-bromo-5-fluoro-2,3-dimethylbenzene (7 g, 1 equiv) in THF (150 mL) was added LDA (2 M, 22.4 mL, 1.3 equiv) at −78° C. The reaction was stirred at −78° C. for 0.5 hours. The mixture was added a solution of DMF (2.77 g, 1.1 equiv) in THF (4 mL) at −78° C. The reaction was stirred at −78° C. for 0.5 hours. The mixture was quenched with aqueous NH₄Cl solution (100 mL) and extracted with ethyl acetate (2×200 mL). The combined organic layers were dried over anhydrous sodium sulfate, concentrated and purified with column chromatography (SiO₂, petroleum ether/ethyl acetate=1/0 to 20/1) to afford the title compound (0.9 g, 11% yield) as yellow solid; LCMS (ESI, M+1, M+3): m/z=231.0, 233.0.

Step C. 4-bromo-5,6-dimethylbenzo[d]isothiazole; A mixture of 2-bromo-6-fluoro-3,4-dimethylbenzaldehyde (0.9 g, 1 equiv) and NH₃·H₂O (5 mL, 28% purity, 9.33 equiv) in 2-methoxyethanol (5 mL) was added Sulfur (125 mg, 1 equiv). The reaction was stirred at 90° C. for 12 hours. The mixture was diluted with ethyl acetate (20 mL) and washed with brine (3×20 mL). The organic layer was dried over anhydrous sodium sulfate, concentrated and purified with column chromatography (SiO₂, petroleum ether/ethyl acetate=1/0 to 10/1) to afford the title compound (600 mg, 34% yield) as yellow solid; LCMS (ESI, M+1, M+3): m/z=242.0, 244.0.

Step D. 5,6-dimethyl-4-(4,4,5,5-tetramethyl-1,3,2-dioxaborolan-2-yl)benzo[d]isothiazol: To a solution of 4-bromo-5,6-dimethyl-1,2-benzothiazole (300 mg, 1.0 equiv) and 4,4,5,5-tetramethyl-2-(4,4,5,5-tetramethyl-1,3,2-dioxaborolan-2-yl)-1,3,2-dioxaborolane (503 mg, 1.6 equiv) in dioxane (5 mL) were added KOAc (365 mg, 3.0 equiv) and PCy₃ Pd G2 (73.2 mg, 0.1 equiv). The reaction was degassed and purged with nitrogen 3 times. The reaction was stirred at 90° C. for 6 hours. The mixture was diluted with H₂O (20 mL) and extracted with EtOAc (3×10 mL). The combined organic layers were washed with brine (10 mL), dried over anhydrous anhydrous sodium sulfate, concentrated and purified with reversed phase flash chromatography [C18, 0.1% formic acid condition] to afford the title compound (120 mg, 31% yield) as red solid; LCMS (ESI, M+1): m/z=290.0.

Step E. (3R)-1-(7-(5,6-dimethylbenzo[d]isothiazol-4-yl)-8-fluoro-2-((hexahydro-1H-pyrrolizin-7a-yl)methoxy)pyrido[4,3-d]pyrimidin-4-yl)-3-methylpiperidin-3-ol: To a solution of (R)-1-(7-chloro-8-fluoro-2-((tetrahydro-1H-pyrrolizin-7a(5H)-yl)methoxy)pyrido[4,3-d]pyrimidin-4-yl)-3-methylpiperidin-3-ol (100 mg, 1.0 equiv) and 5,6-dimethyl-4-(4,4,5,5-tetramethyl-1,3,2-dioxaborolan-2-yl)benzo[d]isothiazol (79.6 mg, 1.2 equiv) in THF (0.5 mL) were added K₃PO₄ (1.5 M in H₂O, 459 μL, 3.0 equiv) and CataCXium A Pd G3 (16.7 mg, 0.1 equiv). The reaction was degassed and purged with nitrogen 3 times. The reaction was stirred at 60° C. for 2 hours. The mixture was diluted with H₂O (10 mL) and extracted with EtOAc (3×10 mL). The combined organic layers were washed with brine (10 mL), dried over anhydrous anhydrous sodium sulfate, concentrated and purified with prep-HPLC [column: Phenomenex luna C18 150×25 mm×10 um; mobile phase: water (FA)-CAN; B %: 14%-44% over 10 minutes] to afford the title compound (62.7 mg, 48% yield, 0.81 HCOOH) as yellow solid; ¹H NMR (400 MHz, METHANOL-d₄) δ=9.35 (d, J=8.8 Hz, 1H), 8.48 (d, J=5.6 Hz, 1H), 8.02 (s, 1H), 4.62 (s, 3H), 4.36-4.33 (m, 1H), 3.69-3.56 (m, 3H), 3.45-3.34 (m, 1H), 3.22-3.18 (m, 2H), 2.54 (s, 3H), 2.32-2.23 (m, 5H), 2.22-2.10 (m, 5H), 2.08-2.00 (m, 2H), 1.90-1.82 (m, 1H), 1.79-1.73 (m, 2H), 1.29 (s, 3H); LCMS (ESI, M+1): m/z=563.2.

### Example 821

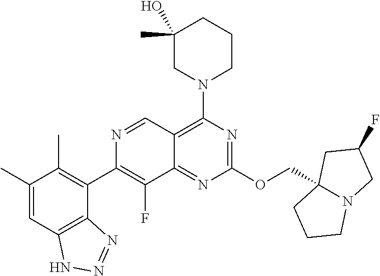 [SMILES: Cc1cc2[nH]nnc2c(-c2ncc3c(N4CCC[C@@](C)(O)C4)nc(OC[C@@]45CCCN4C[C@H](F)C5)nc3c2F)c1C]

### (3R)-1-(7-(5,6-dimethyl-1H-benzo[d][1,2,3]triazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol

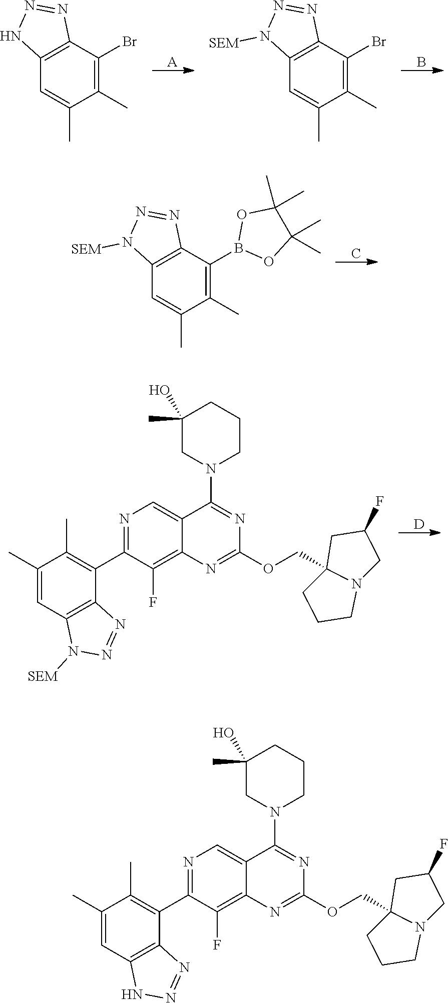

Step A. 4-bromo-5,6-dimethyl-1-((2-(trimethylsilyl) ethoxy)methyl)-1H-benzo[d][1,2,3]triazole: To a mixture of 4-bromo-5,6-dimethyl-1H-benzo[d][1,2,3]triazole (20.0 g, 1.0 equiv) in tetrahydrofuran (200 mL) was added NaH (5.31 g, 60% purity, 1.0 equiv). The reaction was stirred at 0° C. for 1 hour. (2-(chloromethoxy)ethyl)trimethylsilane (29.5 g, 2.0 equiv) was added into above mixture. The reaction was stirred at 0° C. for 3 hours. The mixture was quenched with H₂O (20 mL) at 0° C. and extracted with EtOAc (3×100 mL). The combined organic layers were dried over anhydrous sodium sulfate, concentrated and purified with column chromatography [SiO₂, Petroleum ether/Ethyl acetate=20/1 to 5/1] to afford the title compound (14.5 g, 45% yield) as yellow solid; LCMS (ESI, M+1, M+3): m/z=356.0, 358.0.

Step B. 5,6-dimethyl-4-(4,4,5,5-tetramethyl-1,3,2-dioxaborolan-2-yl)-1-((2-(trimethylsilyl) ethoxy)methyl)-1H-benzo[d][1,2,3]triazole: To a solution of 4-bromo-5,6-dimethyl-1-((2-(trimethylsilyl) ethoxy)methyl)-1H-benzo[d][1,2,3]triazole (12.0 g, 1.0 equiv) and 4,4,4′,4′,5,5,5′,5′-octamethyl-2,2′-bi (1,3,2-dioxaborolane) (12.8 g, 1.5 equiv) in dioxane (120 mL) were added KOAc (9.92 g, 3.0 equiv) and Pd(dppf)Cl₂ (3.70 g, 0.15 equiv). The reaction was degassed and purged with nitrogen 3 times. The mixture was stirred at 100° C. for 12 hours. The mixture was diluted with H₂O (100 mL) and extracted with EtOAc (3×100 mL). The combined organic layers were dried over anhydrous sodium sulfate, concentrated and purified with reversed phase flash chromatography [C18, 0.1% formic acid condition] to afford the title compound (7.00 g, 24% yield) as white solid; LCMS (ESI, M+1): m/z=404.2.

Step C. (3R)-1-(7-(5,6-dimethyl-1-((2-(trimethylsilyl) ethoxy)methyl)-1H-benzo[d][1,2,3]triazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol: To a mixture of (R)-1-(7-chloro-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol (170 mg, 1.0 equiv) and 5,6-dimethyl-4-(4,4,5,5-tetramethyl-1,3,2-dioxaborolan-2-yl)-1-((2-(trimethylsilyl) ethoxy)methyl)-1H-benzo[d][1,2,3]triazole (227 mg, 1.5 equiv) in methoxycyclopentane (2 mL) and H₂O (0.5 mL) were added cataCXium A Pd G3 (40.9 mg, 0.15 equiv) and Cs₂CO₃ (336 mg, 3.0 equiv). The reaction was degassed and purged with nitrogen 3 times. The mixture was stirred at 90° C. for 6 hours. The mixture was diluted with H₂O (5 mL) and extracted with ethyl acetate (3×15 mL). The combined organic layers were dried over anhydrous sodium sulfate, concentrated and purified with reversed phase flash chromatography [C18, 0.1% formic acid condition] to afford the title compound (100 mg, 31% yield) as white solid; LCMS (ESI, M+1): m/z=695.3.

Step D. (3R)-1-(7-(5,6-dimethyl-1H-benzo[d][1,2,3]triazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol: To a solution of (3R)-1-(7-(5,6-dimethyl-1-((2-(trimethylsilyl) ethoxy)methyl)-1H-benzo[d][1,2,3]triazol-4-yl)-8-fluoro-2-(((2R,7aS)-2-fluorohexahydro-1H-pyrrolizin-7a-yl)methoxy)pyrido[4,3-d]pyrimidin-4-yl)-3-methylpiperidin-3-ol (100 mg, 1.0 equiv) in dichloromethane (1 mL) was added 2,2,2-trifluoroacetic acid (164 mg, 10 equiv). The reaction was stirred at 25° C. for 12 hours. The mixture was concentrated. The residue was diluted with water (2 mL). The mixture was adjusted to pH=10 with NaOH solution (10% w/w) at 0° C. and extracted with dichloromethane (3×2 mL). The combined organic layers were dried over anhydrous sodium sulfate, concentrated, and purified with prep-HPLC [Phenomenex luna C18 150×25 mm×10 μm; A: water (FA), B: ACN, B %: 9%-39% over 10 min] to afford the title compound (40.4 mg, 49% yield, 0.78 HCOOH) as white solid; ¹H NMR (400 MHZ, METHANOL-d₄) δ=9.47-9.17 (m, 1H), 8.55-8.28 (m, 1H), 7.92-7.66 (m, 1H), 5.59-5.40 (m, 1H), 4.71-4.48 (m, 3H), 4.40-4.31 (m, 1H), 3.91-3.58 (m, 4H), 3.52-3.33 (m, 1H), 2.68-2.43 (m, 5H), 2.39-2.21 (m, 6H), 2.19-2.06 (m, 2H), 1.91-1.72 (m, 3H), 1.34-1.22 (m, 3H); LCMS (ESI, M+1): m/z=565.3.

### Example 822

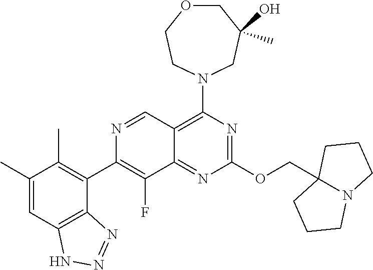 [SMILES: Cc1cc2[nH]nnc2c(-c2ncc3c(N4CCOC[C@@](C)(O)C4)nc(OCC45CCCN4CCC5)nc3c2F)c1C]

### (6S)-4-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-6-methyl-1,4-oxazepan-6-ol

Synthesized according to Example 821. The title compound was obtained as white solid (FA salt). ¹H NMR (400 MHZ, METHANOL-d₄) δ=9.64 (s, 1H), 7.80 (s, 1H), 4.60-4.47 (m, 4H), 4.22-4.12 (m, 1H), 4.05-3.97 (m, 1H), 3.95-3.81 (m, 2H), 3.74-3.63 (m, 2H), 3.51-3.41 (m, 2H), 3.12-3.02 (m, 2H), 2.54 (s, 3H), 2.31-2.26 (m, 3H), 2.26-2.18 (m, 2H), 2.15-2.02 (m, 4H), 2.01-1.92 (m, 2H), 1.29 (s, 3H); LCMS (ESI, M+1): m/z=563.4.

### Example 823

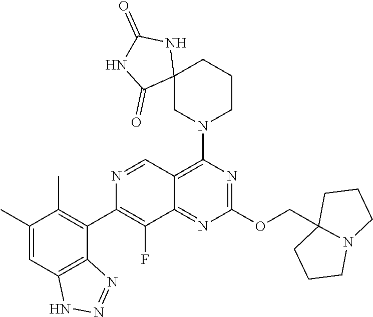 [SMILES: Cc1cc2[nH]nnc2c(-c2ncc3c(N4CCCC5(C4)NC(=O)NC5=O)nc(OCC45CCCN4CCC5)nc3c2F)c1C]

### 7-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione

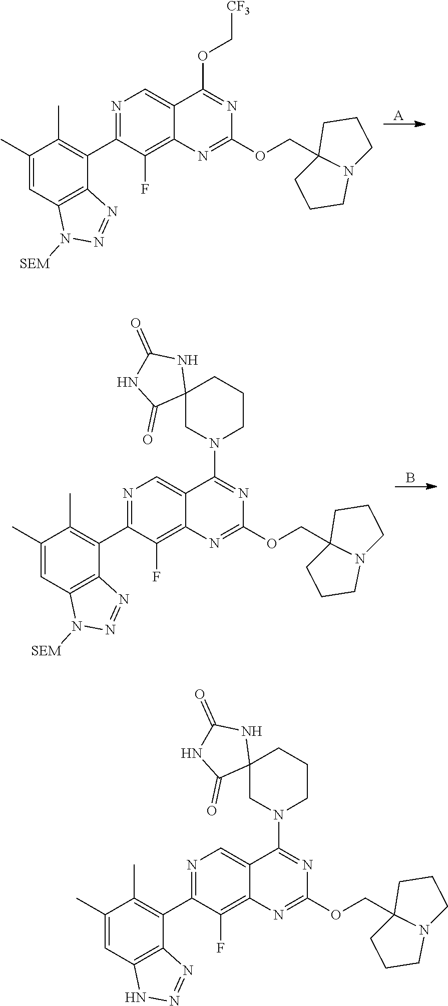

Step A. 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a mixture of 7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)-4-(2,2,2-trifluoroethoxy)pyrido[4,3-d]pyrimidine (Synthesized according to Example 732 step A-B, 150 mg, 1.0 equiv), 1,3,7-triazaspiro[4.5]decane-2,4-dione (76.7 mg, 2.0 equiv) and 4 Å molecular sieve (50.0 mg) in DMF (1 mL) was added DIEA (87.9 mg, 3.0 equiv). The reaction was stirred at 40° C. for 12 hours. The mixture was filtered and purified with reversed phase flash chromatography [C18, 0.1% formic acid condition] to afford the title compound (110 mg, 66% yield) as yellow solid; LCMS (ESI, M+1): m/z=731.1.

Step B. 7-(7-(5,6-dimethyl-1H-benzo[d][1,2,3]triazol-4-yl)-8-fluoro-2-(hexahydro-1H-pyrrolizin-7a-yl)methoxy)pyrido[4,3-d]pyrimidin-4-yl)-1,3,7-triazaspiro[4.5]decane-2,4-dione: To a mixture of 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione (100 mg, 1.0 equiv) in dichloromethane (0.5 mL) was added TFA (1.54 g, 99 equiv) at 0° C. The reaction was stirred at 20° C. for 1 hour. The mixture was adjusted pH to 8 with saturated NaHCO₃ aqueous solution (20 mL) and extracted with dichloromethane (3×10 mL). The combined organic layers were dried over anhydrous sodium sulfate, concentrated and purified with prep-HPLC [Phenomenex C18 75×30 mm×3 μm; A: water (FA), B: ACN, B %: 5%-35% over 7 min] to afford the title compound (13.4 mg, 16% yield, 0.67 HCOOH) as white solid; ¹H NMR (400 MHZ, METHANOL-d₄) δ=9.23 (s, 1H), 7.80 (s, 1H), 4.67-4.56 (m, 3H), 4.51-4.29 (m, 1H), 3.90-3.69 (m, 2H), 3.67-3.56 (m, 2H), 3.25-3.11 (m, 2H), 2.60-2.50 (m, 3H), 2.35-2.22 (m, 6H), 2.22-2.12 (m, 4H), 2.12-2.02 (m, 3H), 2.02-1.91 (m, 2H); LCMS (ESI, M+1): m/z=601.3.

### Example 824

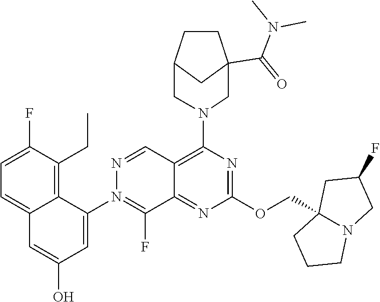

### 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-3-azabicyclo[3.2.1]octane-1-carboxamide

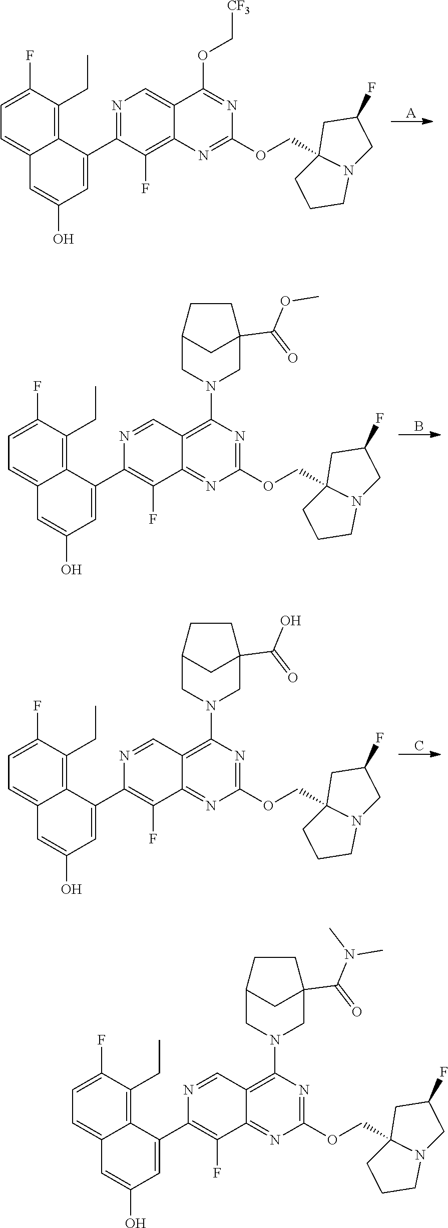

Step A. methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate: To a mixture of methyl 3-azabicyclo[3.2.1]octane-1-carboxylate (320 mg, 1.2 equiv, HCl) and DIEA (670 mg, 4.0 equiv) in DMF (2 mL) was added 4 Å molecular sieve (25 mg). The reaction was stirred at 20° C. for 0.1 hours. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (768 mg, 1.0 equiv) was added into the above mixture. The reaction was stirred at 40° C. for 12 hours. The mixture was filtered and purified by reversed-phase (0.1% FA condition) to afford the title compound (500 mg, 52% yield, 90% purity) as yellow solid; LCMS (ESI, M+1): m/z=662.2.

Step B. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid: To a solution of methyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate (450 mg, 1.0 equiv) in H₂O (0.9 mL) and THF (4.5 mL) was added NaOH (136 mg, 5.0 equiv). The reaction mixture was stirred at 20° C. for 12 hours. The mixture was adjusted pH to 6 by 1 N HCl aqueous at 0° C. and purified by reversed-phase (0.1% formic acid condition) to afford the title compound (130 mg, 29.5% yield) as white solid; LCMS (ESI, M+1): m/z=648.2.

Step C. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-3-azabicyclo[3.2.1]octane-1-carboxamide: To a solution of 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid (30 mg, 1.0 equiv) and TEA (37 mg, 8.0 equiv) in DMF (0.3 mL) were added EDCI (11 mg, 1.2 equiv) and HOBt (12 mg, 2.0 equiv). The reaction was stirred at 20° C. for 0.1 hours. N-methylmethanamine (2 M, 232 μL, 10.0 equiv) was added into the mixture. The reaction was stirred at 40° C. for 12 hours. The mixture was filtered and purified with reversed-phase HPLC (column: Phenomenex Luna C18 150×25 mm×10 um; mobile phase: [water (FA)-ACN]; B %: 16%-46%, 10 minutes) to afford the title compound (20 mg, 63% yield) as white solid; ¹H NMR (400 MHZ, METHANOL-d₄) δ=9.13-9.03 (m, 1H), 7.68 (dd, J=6.0, 9.2 Hz, 1H), 7.30 (d, J=2.8 Hz, 1H), 7.26 (t, J=9.2 Hz, 1H), 7.06 (dd, J=2.8, 8.8 Hz, 1H), 5.52-5.24 (m, 2H), 4.49-4.19 (m, 3H), 3.98-3.84 (m, 1H), 3.69-3.32 (m, 7H), 3.23-2.89 (m, 4H), 2.56-2.35 (m, 3H), 2.35-2.24 (m, 2H), 2.22-2.03 (m, 6H), 2.00-1.81 (m, 3H), 1.59-1.42 (m, 1H), 0.86-0.72 (m, 3H); LCMS (ESI, M+1): m/z=675.5.

### Example 825

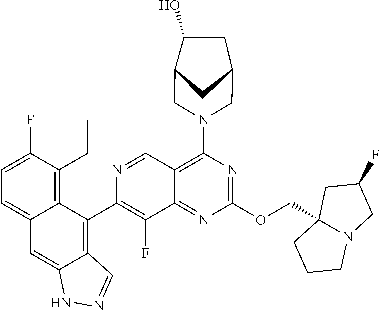 [SMILES: CCc1c(F)ccc2cc3[nH]ncc3c(-c3ncc4c(N5C[C@@H]6C[C@H](C5)[C@H](O)C6)nc(OC[C@@]56CCCN5C[C@H](F)C6)nc4c3F)c12]

### (1R,5R,6R)-3-(7-(5-ethyl-6-fluoro-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

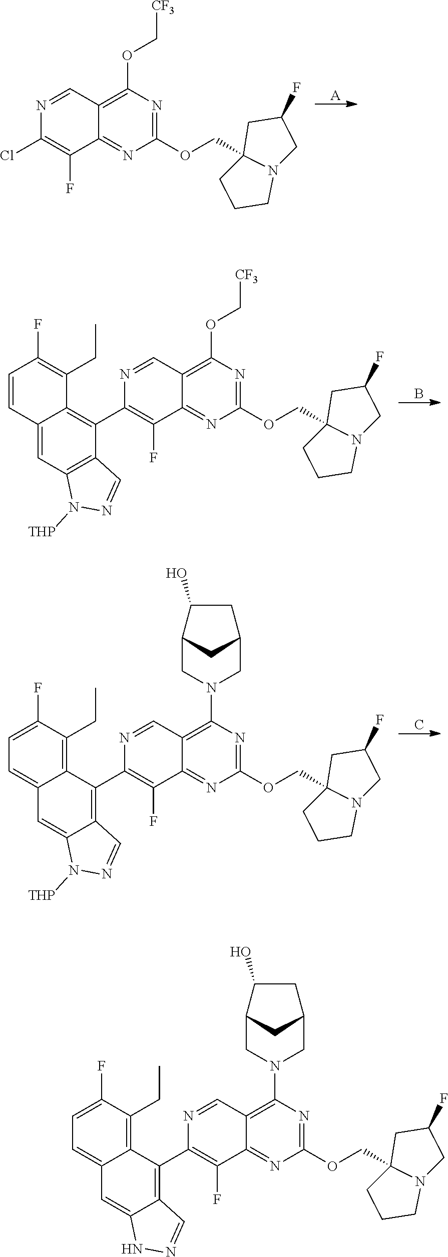

Step A. 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole: To a mixture of 7-chloro-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (20.7 mg, 1.0 equiv) and 5-ethyl-6-fluoro-1-(tetrahydro-2H-pyran-2-yl)-4-(4,4,5,5-tetramethyl-1,3,2-dioxaborolan-2-yl)-1H-benzo[f]indazole (20.0 mg, 1.0 equiv) in Methoxy cyclopentane (4 mL) and H₂O (1.3 mL) were added CataCXium A Pd G3 (3.43 mg, 0.10 equiv) and Cs₂CO₃ (46.1 mg, 3.0 equiv). The reaction was degassed and purged with nitrogen 3 times. The reaction was stirred at 80° C. for 12 hours. The mixture was filtered and purified by reversed-phase HPLC (0.1% FA condition) to afford the title compound (9.00 mg, 24% yield) as yellow solid; LCMS (ESI, M+1): m/z=701.2.

Step B. (1R,5R,6R)-3-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole (20.0 mg, 1.0 equiv) and (1R,5R,6R)-3-azabicyclo[3.2.1]octan-6-ol (7.26 mg, 2.0 equiv) in DMF (1 mL) were added DIEA (22.1 mg, 6.0 equiv) and 4 Å molecular sieve (2.00 mg). The reaction was stirred at 60° C. for 2 hours. The mixture was filtered and purified by reversed-phase HPLC [water (FA, 0.1%)/acetonitrile] to afford the title compound (20.0 mg, 93% yield) as yellow solid; LCMS (ESI, M+1): m/z=728.7.

Step C. (1R,5R,6R)-3-(7-(5-ethyl-6-fluoro-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol: A mixture of (1R,5R,6R)-3-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-6-ol (15.0 mg, 1.0 equiv) in TFA (578 mg, 246 equiv) was stirred at 20° C. for 0.5 hour. The mixture was concentrated. The residue was diluted with water (1 mL). The mixture was adjusted to pH=7 with saturated NaHCO₃ (10 mL) and extracted with ethyl acetate (5 mL×3). The combined organic layers and dried over by anhydrous sodium sulfate, concentrated, and purified with prep-HPLC (column: Phenomenex luna C18 150× 25 mm×10 um; mobile phase: [water (FA)-ACN]; B %: 18%-48%, 10 min) to afford the title compound (6.03 mg, 45% yield, 0.17 HCOOH) as yellow solid. ¹H NMR (400 MHZ, METHANOL-d4) δ=9.39-9.30 (m, 1H), 8.24 (s, 1H), 8.01 (dd, J=6.0, 9.2 Hz, 1H), 7.72 (s, 1H), 7.33 (dt, J=1.6, 9.2 Hz, 1H), 5.44-5.23 (m, 1H), 5.09-5.00 (m, 1H), 4.79 (br s, 1H), 4.42-4.23 (m, 3H), 3.90-3.76 (m, 1H), 3.57-3.36 (m, 2H), 3.27 (br d, J=7.6 Hz, 1H), 3.12-3.00 (m, 1H), 2.65 (br dd, J=7.2, 14.8 Hz, 1H), 2.46-2.37 (m, 2H), 2.36-2.14 (m, 5H), 2.06-1.79 (m, 5H), 1.43 (br d, J=13.6 Hz, 1H), 0.92-0.80 (m, 3H); LCMS (ESI, M+1): m/z=644.2.

### Example 826

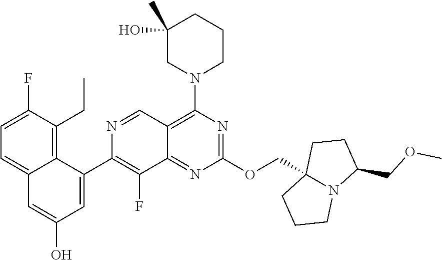 [SMILES: CCc1c(F)ccc2cc(O)cc(-c3ncc4c(N5CCC[C@@](C)(O)C5)nc(OC[C@@]56CCCN5[C@H](COC)CC6)nc4c3F)c12]

### (R)-1-(7-(8-ethyl-7-fluoro-3-hydroxynaphthalen-1-yl)-8-fluoro-2-(((3S,7aS)-3-(methoxymethyl)tetrahydro-1H-pyrrolizin-7a(5H)-yl)methoxy)pyrido[4,3-d]pyrimidin-4-yl)-3-methylpiperidin-3-ol

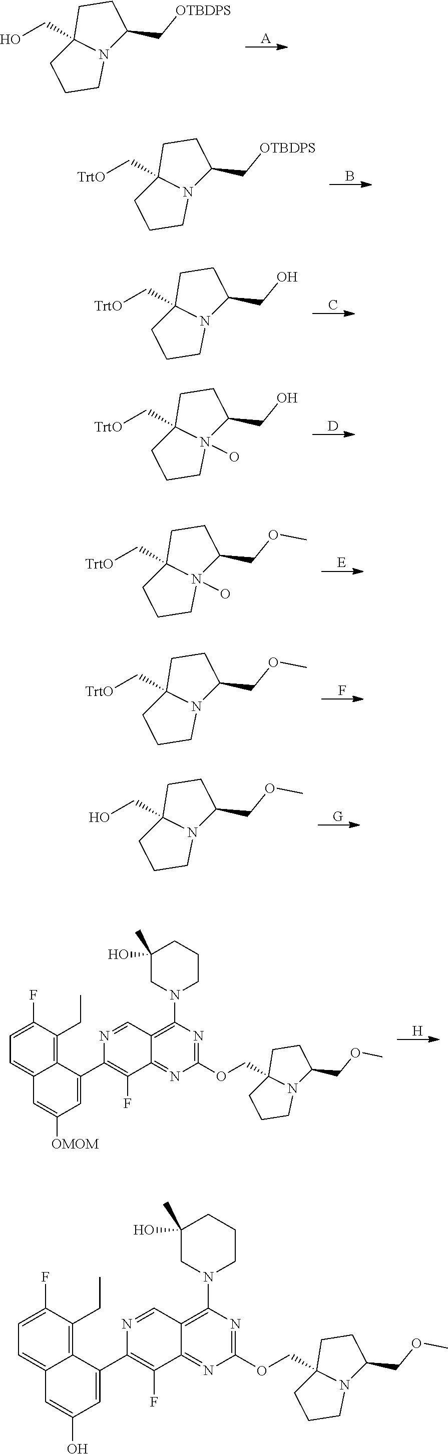

Step A. (3S,7aS)-3-(((tert-butyldiphenylsilyl)oxy)methyl)-7a-((trityloxy)methyl) hexahydro-1H-pyrrolizine: To a solution of ((3S,7aS)-3-(((tert-butyldiphenylsilyl)oxy)methyl) hexahydro-1H-pyrrolizin-7a-yl)methanol (6.00 g, 1.0 equiv) and TEA (2.96 g, 2.0 equiv) in DCM (100 mL) was added TrtCl (6.12 g, 1.5 equiv). The reaction was stirred at 25° C. for 12 hours. The mixture was diluted with water (50 mL) and extracted with DCM (3× 50 mL). The combined organic layers were washed with brine (3×50 mL), dried over sodium sulfate, concentrated and purified with reversed phase flash chromatography [C18, 0.1% formic acid condition] to afford the title compound (3.60 g, 38% yield) as a yellow oil.

Step B. ((3S,7aS)-7a-((trityloxy)methyl) hexahydro-1H-pyrrolizin-3-yl)methanol: To a solution of (3S,7aS)-3-(((tert-butyldiphenylsilyl)oxy)methyl)-7a-((trityloxy)methyl) hexahydro-1H-pyrrolizine (2.00 g, 1.0 equiv) in DMF (20 mL) was added CsF (6.99 g, 15 equiv). The reaction was stirred at 40° C. for 12 hours. The mixture was filtered, the filtrate was concentrated and purified by reversed phase flash chromatography [C18, 0.1% formic acid condition] to afford the title compound (0.80 g, 63% yield) as a yellow oil; LCMS (ESI, M+1): m/z=414.2.

Step C. (3S,7aS)-3-(hydroxymethyl)-7a-((trityloxy)methyl) octahydropyrrolizine 4-oxide: To a solution of ((3S,7aS)-7a-((trityloxy)methyl) hexahydro-1H-pyrrolizin-3-yl)methanol (250 mg, 1.0 equiv) in DCM (3 mL) was added m-CPBA (147 mg, 1.2 equiv). The reaction was stirred at 25° C. for 2 hours. The mixture was quenched by addition of saturated NaHCO₃ aqueous solution (2.5 mL) and extracted with DCM (10 mL). The combined organic layers were washed with saturated sodium bicarbonate aqueous solution (10 mL) and brine (10 mL), dried over sodium sulfate, filtered and concentrated to afford the title compound (230 mg, crude) as a white solid; LCMS (ESI, M+1): m/z=430.2.

Step D. (3S,7aS)-3-(methoxymethyl)-7a-((trityloxy)methyl) octahydropyrrolizine 4-oxide: To a solution of (3S,7aS)-3-(hydroxymethyl)-7a-((trityloxy)methyl) octahydropyrrolizine 4-oxide (460 mg, 1.0 equiv) in DMF (5 mL) was added NaH (64.3 mg, 60% purity, 1.5 equiv). The mixture was stirred at 0° C. for 30 minutes. MeI (228 mg, 1.5 equiv) was added. The reaction was stirred at 0° C. for 2 hours. The mixture was quenched by addition of ice-water (15 mL) and extracted with ethyl acetate (2×15 mL). The combined organic layers were washed with brine (15 mL), dried over sodium sulfate, concentrated and purified by prep-HPLC [column: Waters Xbridge 150×25 mm×5 μm; A: water (NH₃·H₂O), B: ACN, B %: 44%-74% over 9 min] to afford the title compound (130 mg, 26% yield) as a yellow solid; LCMS (ESI, M+1): m/z=444.2.

Step E. (3S,7aS)-3-(methoxymethyl)-7a-((trityloxy)methyl) hexahydro-1H-pyrrolizine: To a solution of (3S,7aS)-3-(methoxymethyl)-7a-((trityloxy)methyl) octahydropyrrolizine 4-oxide (30.0 mg, 1.0 equiv) in MeOH (0.6 mL) was added Pd/C (5.00 mg, 10% purity) under N₂ atmosphere. The suspension was degassed, purged with H₂ for 3 times and stirred at 25° C. for 12 hours under H₂ (15 Psi) atmosphere. The mixture was filtered through Celite and washed with MeOH (10 mL). The filtrate was concentrated to afford the title compound (25.0 mg, crude) as a yellow solid; LCMS (ESI, M+1): m/z=428.2.

Step F. ((3S,7aS)-3-(methoxymethyl) hexahydro-1H-pyrrolizin-7a-yl)methanol: To a solution of (3S,7aS)-3-(methoxymethyl)-7a-((trityloxy)methyl) hexahydro-1H-pyrrolizine (25.0 mg, 1.0 equiv) in DCM (0.3 mL) was added TFA (156 uL). The reaction was stirred at 20° C. for 2 hours. The mixture was concentrated to afford the title compound (25.0 mg, crude) as a yellow solid, which was used directly for the next step without further purification.

Step G. (R)-1-(7-(8-ethyl-7-fluoro-3-(methoxymethoxy)naphthalen-1-yl)-8-fluoro-2-(((3S,7aS)-3-(methoxymethyl) hexahydro-1H-pyrrolizin-7a-yl)methoxy)pyrido[4,3-d]pyrimidin-4-yl)-3-methylpiperidin-3-ol: A mixture of ((3S,7aS)-3-(methoxymethyl) hexahydro-1H-pyrrolizin-7a-yl)methanol (21.0 mg, 1.2 equiv), 4 Å molecular sieve (5 mg) and t-BuONa (36.3 mg, 4.0 equiv) in toluene (0.5 mL) was stirred at 0° C. for 10 minutes. (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (50.0 mg, 1.0 equiv) was added. The reaction was stirred at 0° C. for 1 hour. The mixture was quenched with H₂O (10 mL) at 0° C. and extracted with ethyl acetate (3×10 mL). The combined organic layers were washed with brine (20 mL), dried over sodium sulfate, concentrated and purified with prep-HPLC [column: Phenomenex luna C18 75×30 mm×3 um; A: water (FA), B: ACN, B %: 24%-44% over 8 min] to afford the title compound (15.0 mg, 23% yield) as a white solid; LCMS (ESI, M+1): m/z=678.5.

Step H. (R)-1-(7-(8-ethyl-7-fluoro-3-hydroxynaphthalen-1-yl)-8-fluoro-2-(((3S,7aS)-3-(methoxymethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethyl-7-fluoro-3-(methoxymethoxy)naphthalen-1-yl)-8-fluoro-2-(((3S,7aS)-3-(methoxymethyl) hexahydro-1H-pyrrolizin-7a-yl)methoxy)pyrido[4,3-d]pyrimidin-4-yl)-3-methylpiperidin-3-ol (10.0 mg, 1 equiv) in MeOH (0.2 mL) was added HCl/MeOH (4 M, 200 uL) at 0° C. The reaction was stirred at 25° C. for 0.5 hour. The mixture was concentrated, diluted with MeCN (0.5 mL) and adjusted to PH˜7 with 10% NH₃·H₂O. The mixture was directly purified by prep-HPLC [column: Phenomenex luna C18 150×25 mm×10 μm; A: water (FA), B: ACN, B %: 19%-49% over 10 min] to afford the title compound (6.00 mg, 62% yield) as a yellow solid. ¹H NMR (400 MHZ, METHANOL-d₄) δ=9.29-9.17 (m, 1H), 8.55 (s, 1H), 7.75-7.62 (m, 1H), 7.31 (t, J=2.8 Hz, 1H), 7.25 (dt, J=1.6, 9.2 Hz, 1H), 7.06 (s, 1H), 4.64-4.42 (m, 3H), 4.39-4.24 (m, 2H), 3.69-3.56 (m, 2H), 3.50-3.41 (m, 2H), 3.39 (d, J=1.6 Hz, 1H), 3.34 (d, J=4.8 Hz, 2H), 3.21-2.92 (m, 2H), 2.54-2.40 (m, 1H), 2.35-1.71 (m, 13H), 1.34-1.25 (m, 3H), 0.88-0.75 (m, 3H); LCMS (ESI, M+1): m/z=634.3.

### Example 827

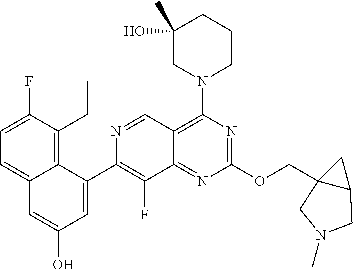 [SMILES: CCc1c(F)ccc2cc(O)cc(-c3ncc4c(N5CCC[C@@](C)(O)C5)nc(OCC56CC5CN(C)C6)nc4c3F)c12]

### (3R)-1-(7-(8-ethyl-7-fluoro-3-hydroxynaphthalen-1-yl)-8-fluoro-2-((3-methyl-3-azabicyclo[3.1.0]hexan-1-yl)methoxy)pyrido[4,3-d]pyrimidin-4-yl)-3-methylpiperidin-3-ol

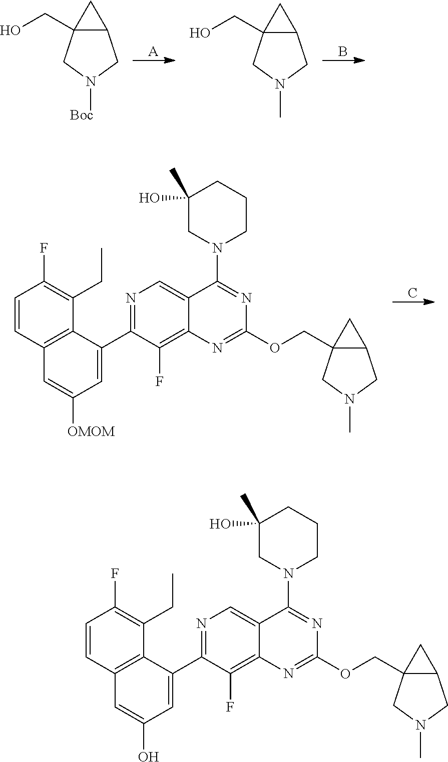

Step A. (3-methyl-3-azabicyclo[3.1.0]hexan-1-yl)methanol: To a solution of tert-butyl 1-(hydroxymethyl)-3-azabicyclo[3.1.0]hexane-3-carboxylate (500 mg, 1.0 equiv) in THF (8.0 mL) was added LAH (222 mg, 2.5 equiv) at 0° C. The reaction was stirred at 60° C. for 1 hour. The mixture was quenched with water (0.2 mL), 15% NaOH solution (0.2 mL) and water (0.6 mL) at 0° C. The mixture was diluted with EtOAc (20 mL) and filtered. The filtrate was dried, filtered, concentrated and purified with prep-TLC (SiO₂, methylene chloride/methanol=5:1) to afford the title compound (200 mg, 67% yield)) as colorless oil; ¹H NMR (400 MHZ, CHLOROFORM-d) δ=4.67 (br s, 1H), 3.76-3.69 (m, 1H), 3.65-3.59 (m, 1H), 3.07 (d, J=8.8 Hz, 1H), 2.96 (d, J=8.8 Hz, 1H), 2.43-2.36 (m, 2H), 2.32 (s, 3H), 1.27 (td, J=4.0, 8.0 Hz, 1H), 1.02 (t, J=4.4 Hz, 1H), 0.49 (dd, J=4.4, 8.0 Hz, 1H).

Step B. (3R)-1-(7-(8-ethyl-7-fluoro-3-(methoxymethoxy)naphthalen-1-yl)-8-fluoro-2-((3-methyl-3-azabicyclo[3.1.0]hexan-1-yl)methoxy)pyrido[4,3-d]pyrimidin-4-yl)-3-methylpiperidin-3-ol: To a solution of (3-methyl-3-azabicyclo[3.1.0]hexan-1-yl)methanol (50.0 mg, 1.0 equiv) in THF (2.0 mL) was added t-BuOK (1.0 M, 2.2 equiv). The reaction was stirred at 20° C. for 0.5 hours. The mixture was added to the solution of (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (145 mg, 0.7 equiv) in THF (2.0 mL). The reaction was stirred at 60° C. for 5 hours. The mixture was quenched with water (0.2 mL), concentrated and purified with prep-HPLC [column: Phenomenex C18 75×30 mm×3 um; mobile phase: water (FA)-ACN; B %: 15%-45%, 7 min] to afford the title compound (40.0 mg, 16% yield) as white solid; LCMS (ESI, M+1): m/z=620.0

Step C. (3R)-1-(7-(8-ethyl-7-fluoro-3-hydroxynaphthalen-1-yl)-8-fluoro-2-((3-methyl-3-azabicyclo[3.1.0]hexan-1-yl)methoxy)pyrido[4,3-d]pyrimidin-4-yl)-3-methylpiperidin-3-ol: To a solution of (3R)-1-[7-[8-ethyl-7-fluoro-3-(methoxymethoxy)-1-naphthyl]-8-fluoro-2-[(3-methyl-3-azabicyclo[3.1.0]hexan-1-yl)methoxy]pyrido[4,3-d]pyrimidin-4-yl]-3-methyl-piperidin-3-ol (20.0 mg, 1.0 equiv) in ACN (1 mL) was added HCl·dioxane (4 M, 1.0 equiv). The reaction was stirred at 0° C. for 1 hour. The mixture was concentrated and purified with prep-HPLC [column: Phenomenex C18 75×30 mm×3 um; mobile phase: [water (FA)-ACN]; B %: 12%-42%, 5 min] to afford the title compound (4.2 mg, 22% yield, HCOOH salt) as white solid; ¹H NMR (400 MHZ, METHANOL-d4) δ=9.22 (s, 1H), 8.52 (s, 1H), 7.68 (dd, J=6.0, 8.8 Hz, 1H), 7.31 (d, J=2.8 Hz, 1H), 7.25 (t, J=9.6 Hz, 1H), 7.06 (s, 1H), 4.68-4.50 (m, 4H), 4.38-4.21 (m, 1H), 3.68-3.50 (m, 2H), 3.50-3.36 (m, 2H), 3.26 (br d, J=10.4 Hz, 1H), 3.20-3.10 (m, 1H), 2.71 (s, 3H), 2.57-2.39 (m, 1H), 2.30-2.07 (m, 2H), 1.91-1.68 (m, 4H), 1.29 (d, J=10.0 Hz, 3H), 1.15-0.91 (m, 2H), 0.81 (q, J=7.6 Hz, 3H); LCMS (ESI, M+1): m/z=576.4.

### Example 828

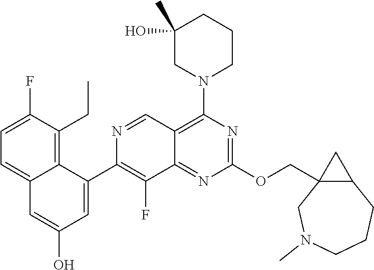 [SMILES: CCc1c(F)ccc2cc(O)cc(-c3ncc4c(N5CCC[C@@](C)(O)C5)nc(OCC56CC5CCCN(C)C6)nc4c3F)c12]

### (3R)-1-(7-(8-ethyl-7-fluoro-3-hydroxynaphthalen-1-yl)-8-fluoro-2-((3-methyl-3-azabicyclo[5.1.0]octan-1-yl)methoxy)pyrido[4,3-d]pyrimidin-4-yl)-3-methylpiperidin-3-ol

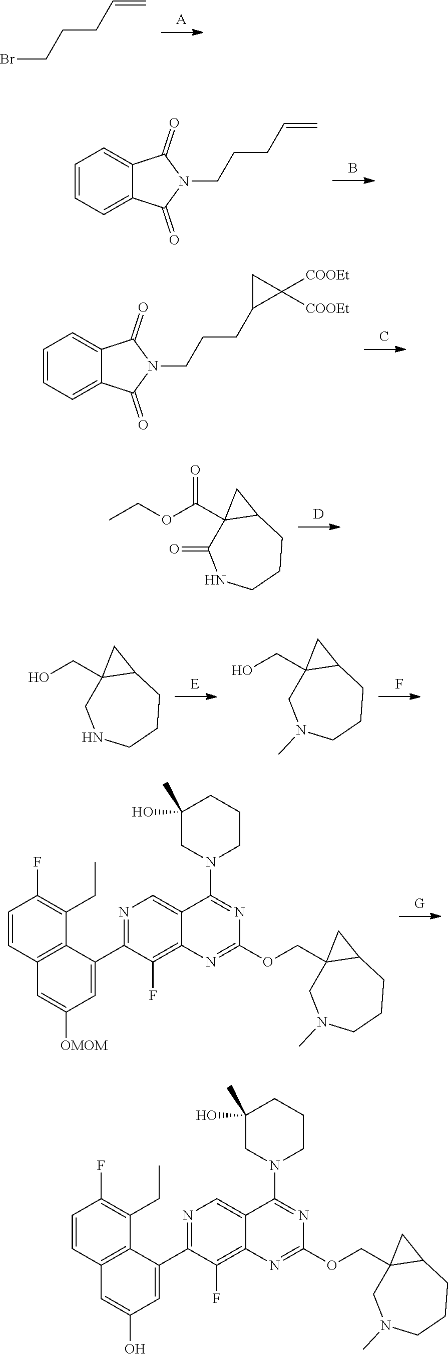

Step A. 2-(pent-4-en-1-yl) isoindoline-1,3-dione: To a solution of 5-bromopent-1-ene (4.0 g, 1.0 equiv) in DMF (30 mL) were added K₂CO₃ (11.1 g, 3.0 equiv) and isoindoline-1,3-dione (4.74 g, 1.2 equiv). The reaction was stirred at 20° C. for 12 hours. The mixture was diluted with water (400 mL) and extracted with ethyl acetate (2× 300 mL). The combined organic layers were purified with column chromatography [SiO₂, petroleum ether/ethyl acetate=20/1 to 3/1] to afford the title compound (5.6 g, 96% yield) as yellow oil; LCMS (ESI, M+1): m/z=216.1.

Step B. diethyl 2-(3-(1,3-dioxoisoindolin-2-yl) propyl)cyclopropane-1,1-dicarboxylate: To a solution of 2-(pent-4-en-1-yl) isoindoline-1,3-dione (1.5 g, 1.0 equiv) in DCM (20 mL) were added dropwise PhCl (1.57 g, 1.41 mL, 2.0 equiv) and Rh₂(OAc)₄ (308 mg, 0.1 equiv) at 25° C., and then diethyl 2-diazopropanedioate (1.56 g, 1.2 equiv) was added dropwise at 60° C. The reaction was stirred at 60° C. for 1 hour. Then diethyl 2-diazopropanedioate (1.56 g, 1.2 equiv) was added dropwise at 60° C. The resulting mixture was stirred at 60° C. for 11 hours. The mixture was diluted with water (50 mL) and extracted with ethyl acetate (2×100 mL). The combined layer was purified with column chromatography [SiO₂, petroleum ether/ethyl acetate=10/1 to 1/1] to afford the title compound (2.4 g, 69% yield) as white solid; LCMS (ESI, M+1): m/z=374.1.

Step C. ethyl 2-oxo-3-azabicyclo[5.1.0]octane-1-carboxylate: To a solution of diethyl 2-(3-(1,3-dioxoisoindolin-2-yl) propyl)cyclopropane-1,1-dicarboxylate (1.0 g, 1.0 equiv) in MeOH (10 mL) was added N₂H₄·H₂O (410 mg, 98% purity, 3.0 equiv). The reaction was stirred at 60° C. for 12 hours. The mixture was diluted with water (40 mL) and extracted with ethyl acetate (2×30 mL). The combined layers were washed with brine (40 mL), dried, concentrated and purified with column chromatography [SiO₂, petroleum ether/ethyl acetate=5/1 to 1/1] to afford the title compound (200 mg, 37% yield) as yellow oil; LCMS (ESI, M+1): m/z=198.2.

Step D. (3-azabicyclo[5.1.0]octan-1-yl)methanol: To a solution of ethyl 2-oxo-3-azabicyclo[5.1.0]octane-1-carboxylate (200 mg, 1.0 equiv) in THF (5 mL) was added LiAlH₄ (57.7 mg, 3.0 equiv). The reaction was stirred at 40° C. for 3 hours. The mixture was quenched with water (10 mL) at 25° C., and purified with prep-TLC [SiO₂, dichloromethane/methanol=10:1] to afford the title compound (120 mg, 82% yield) as white solid; LCMS (ESI, M+1): m/z=142.1.

Step E. (3-methyl-3-azabicyclo[5.1.0]octan-1-yl)methanol: To a solution of 3-azabicyclo[5.1.0]octan-1-ylmethanol (60.0 mg, 1.0 equiv) in DMF (1 mL) were added K₂CO₃ (29.4 mg, 1 equiv) and MeI (30.2 mg, 1.0 equiv). The reaction was stirred at 25° C. for 2 hours. The mixture was concentrated and purified with prep-TLC [SiO₂, dichloromethane/methanol=10:1] to afford the title compound (64 mg, 95% yield) as white solid; LCMS (ESI, M+1): m/z=156.0.

Step F. (3R)-1-(7-(8-ethyl-7-fluoro-3-(methoxymethoxy)naphthalen-1-yl)-8-fluoro-2-((3-methyl-3-azabicyclo[5.1.0]octan-1-yl)methoxy)pyrido[4,3-d]pyrimidin-4-yl)-3-methylpiperidin-3-ol: To a solution of (3-methyl-3-azabicyclo[5.1.0]octan-1-yl)methanol (32 mg, 1.0 equiv) in THF (3 mL) was added dropwise t-BuONa (1 M, 600 μL, 3 equiv) at 0° C. After addition, the mixture was stirred at this temperature for 0.5 hours, and then (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (109 mg, 1.0 equiv) was added. The reaction was stirred at 0° C. for 0.5 hours. The mixture was concentrated and purified with prep-TLC [SiO₂, petroleum ether/ethyl acetate=0:1] to afford the title compound (30 mg, 22% yield) as yellow solid; LCMS (ESI, M+1): m/z=648.4.

Step G. (3R)-1-(7-(8-ethyl-7-fluoro-3-hydroxynaphthalen-1-yl)-8-fluoro-2-((3-methyl-3-azabicyclo[5.1.0]octan-1-yl)methoxy)pyrido[4,3-d]pyrimidin-4-yl)-3-methylpiperidin-3-ol: To a solution of (3R)-1-(7-(8-ethyl-7-fluoro-3-(methoxymethoxy)naphthalen-1-yl)-8-fluoro-2-((3-methyl-3-azabicyclo[5.1.0]octan-1-yl)methoxy)pyrido[4,3-d]pyrimidin-4-yl)-3-methylpiperidin-3-ol (30.0 mg, 1.0 equiv) in HCl·dioxane (2.0 mL). The reaction was stirred at 0° C. for 1 hour. The mixture was filtered and concentrated and purified with prep-HPLC [column: 3_Phenomenex Luna C18 75×30 mm×3 um; mobile phase: [water (FA)-ACN]; B %: 20%-40%, 8 min] to afford the title compound (3.8 mg, 12% yield) as white solid; ¹H NMR (400 MHZ, DMSO-d₆) δ=9.25 (d, J=4.4 Hz, 1H), 8.48 (s, 1H), 7.76 (dd, J=5.6, 9.2 Hz, 1H), 7.43-7.27 (m, 2H), 7.03 (s, 1H), 4.85-4.63 (m, 1H), 4.57-4.33 (m, 3H), 4.22-4.00 (m, 2H), 3.18 (s, 4H), 2.70-2.63 (m, 3H), 2.32 (br d, J=2.0 Hz, 3H), 2.26-1.84 (m, 7H), 1.76-1.58 (m, 4H), 1.17 (d, J=10.0 Hz, 3H), 0.80-0.69 (m, 3H); LCMS (ESI, M+1): m/z=604.4.

### Example 829

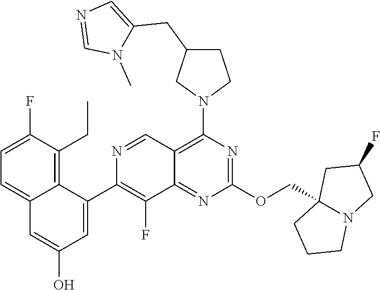 [SMILES: CCc1c(F)ccc2cc(O)cc(-c3ncc4c(N5CCC(Cc6cncn6C)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(3-((1-methyl-1H-imidazol-5-yl)methyl) pyrrolidin-1-yl)pyrido[4,3-d]pyrimidin-7-yl)naphthalen-2-ol

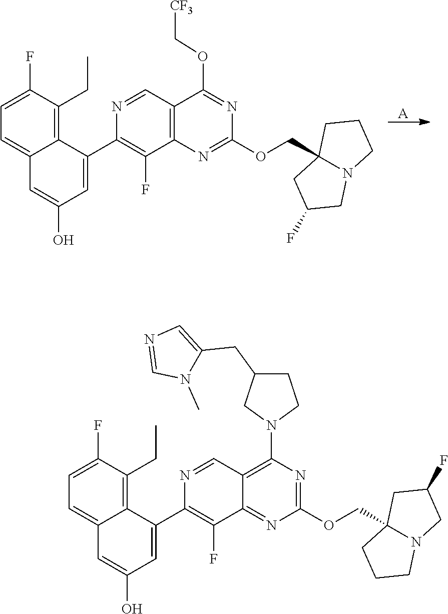

Step A. 5-ethyl-6-fluoro-4-(8-fluoro-2-(((2R,7aS)-2-fluorotetrahydro-1H-pyrrolizin-7a(5H)-yl)methoxy)-4-(3-((1-methyl-1H-imidazol-5-yl)methyl) pyrrolidin-1-yl)pyrido[4,3-d]pyrimidin-7-yl)naphthalen-2-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (60.0 mg, 1.0 equiv) and 1-methyl-5-(pyrrolidin-3-ylmethyl)-1H-imidazole (41.8 mg, 2.5 equiv) in DMF (1 mL) were added 4 Å molecular sieve (10.0 mg, 1.0 equiv) and K₃PO₄ (215 mg, 10.0 equiv). The reaction was stirred at 60° C. for 2 hours. The mixture was filtered and purified with prep-HPLC [column: Waters Xbridge 150×25 mm×5 um; mobile phase: [water (ammonia hydroxide v/v)-ACN]; B %: 35%-65%, 9 min] to afford the title compound (13.4 mg, 20% yield) as off-white solid; ¹H NMR (400 MHZ, METHANOL-d4) δ=9.31 (br s, 1H), 8.25 (br s, 1H), 7.69 (dd, J=5.6, 8.8 Hz, 1H), 7.32 (d, J=2.0 Hz, 1H), 7.26 (t, J=9.2 Hz, 1H), 7.21 (br s, 1H), 7.04 (br d, J=4.8 Hz, 1H), 5.69-5.42 (m, 1H), 4.76-4.60 (m, 3H), 4.45-4.11 (m, 3H), 3.92-3.77 (m, 6H), 3.50-3.39 (m, 1H), 3.03-2.91 (m, 2H), 2.88-2.77 (m, 1H), 2.77-2.63 (m, 1H), 2.63-2.55 (m, 1H), 2.54-2.23 (m, 6H), 2.21-2.09 (m, 2H), 2.03-1.88 (m, 1H), 0.84-0.73 (m, 3H); LCMS (ESI, M+1): m/z=658.4.

### Example 830

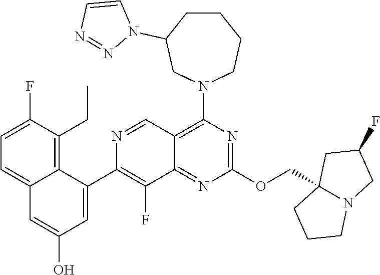 [SMILES: CCc1c(F)ccc2cc(O)cc(-c3ncc4c(N5CCCCC(n6ccnn6)C5)nc(OC[C@@]56CCCN5C[C@H](F)C6)nc4c3F)c12]

### 4-(4-(3-(1H-1,2,3-triazol-1-yl) azepan-1-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

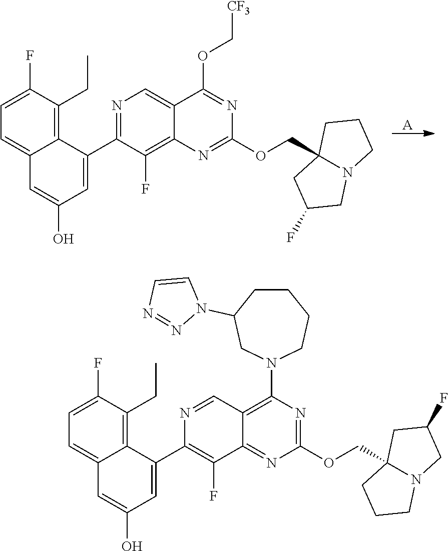

Synthesized according to Example 829. The title compound was obtained as white solid. ¹H NMR (400 MHZ, METHANOL-d₄) δ=9.26 (d, J=2.0 Hz, 1H), 8.69-8.40 (m, 1H), 8.23 (d, J=1.6 Hz, 1H), 7.82 (s, 1H), 7.68 (dd, J=6.0, 8.8 Hz, 1H), 7.31 (d, J=2.4 Hz, 1H), 7.26 (br t, J=8.8 Hz, 1H), 7.09-7.01 (m, 1H), 5.51-5.22 (m, 2H), 4.74-4.58 (m, 4H), 4.51-4.31 (m, 2H), 4.29-4.08 (m, 3H), 3.18-3.02 (m, 1H), 2.56-2.36 (m, 2H), 2.33-1.89 (m, 11H), 1.64-1.43 (m, 1H), 0.87-0.72 (m, 3H); LCMS (ESI, M+1): m/z=659.4.

### Example 831

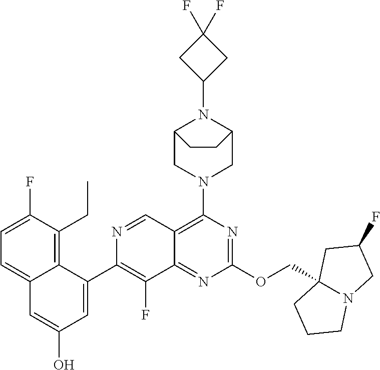 [SMILES: CCc1c(F)ccc2cc(O)cc(-c3ncc4c(N5CC6CCC(C5)N6C5CC(F)(F)C5)nc(OC[C@@]56CCCN5C[C@H](F)C6)nc4c3F)c12]

### 4-(4-((1R,5S)-8-(3,3-difluorocyclobutyl)-3,8-diazabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

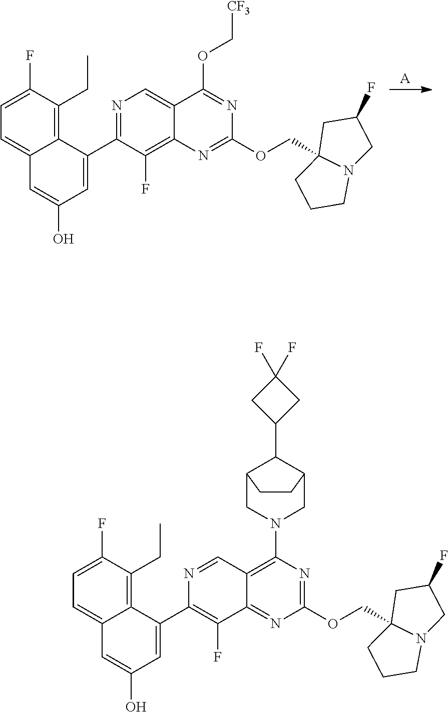

Synthesized according to Example 829. The title compound was obtained as white solid. ¹H NMR (400 MHZ, METHANOL-d₄) δ=9.08 (s, 1H), 8.51 (br s, 1H), 7.68 (dd, J=5.6, 9.2 Hz, 1H), 7.44-7.16 (m, 2H), 7.05 (d, J=2.4 Hz, 1H), 5.56-5.21 (m, 1H), 4.59 (s, 5H), 4.46-4.30 (m, 2H), 3.75 (br d, J=6.4 Hz, 2H), 3.42 (br d, J=2.4 Hz, 4H), 3.18-3.09 (m, 1H), 3.08-2.97 (m, 1H), 2.85-2.69 (m, 2H), 2.54-2.43 (m, 3H), 2.24-2.13 (m, 2H), 2.08 (br d, J=4.4 Hz, 2H), 2.04-1.93 (m, 3H), 1.68 (br d, J=8.8 Hz, 2H), 0.85-0.73 (m, 3H); LCMS (ESI, M+1): m/z=695.4.

### Example 832

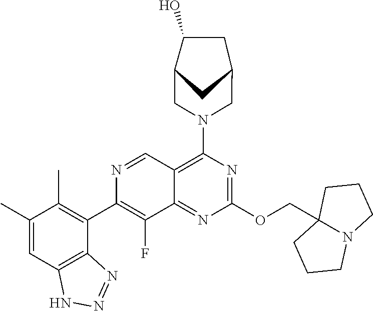 [SMILES: Cc1cc2[nH]nnc2c(-c2ncc3c(N4C[C@@H]5C[C@H](C4)[C@H](O)C5)nc(OCC45CCCN4CCC5)nc3c2F)c1C]

### (1R,5R,6R)-3-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol

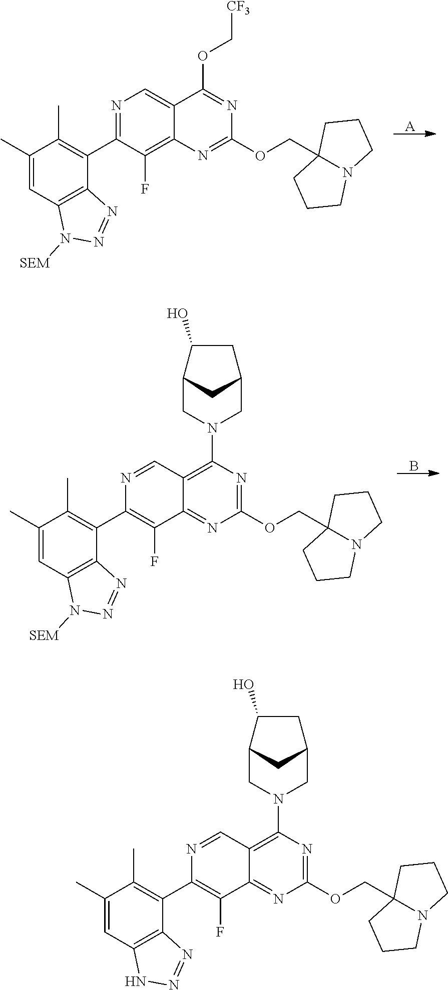

Step A. (1R,5R,6R)-3-(7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol: To a solution of 7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)-4-(2,2,2-trifluoroethoxy)pyrido[4,3-d]pyrimidine (200 mg, 1.0 equiv) and (1R,5R,6R)-3-azabicyclo[3.2.1]octan-6-ol (76.9 mg, 2.0 equiv) in DMF (0.5 mL) was added DIEA (117 mg, 3.0 equiv). The reaction was stirred at 40° C. for 12 hours. The mixture was filtered and purified with reversed-phase HPLC [C18, 0.1% formic acid condition] to afford the title compound (140 mg, 67% yield) as white solid; LCMS (ESI, M+1): m/z=689.4.

Step B. (1R,5R,6R)-3-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-3-azabicyclo[3.2.1]octan-6-ol (130 mg, 1.0 equiv) in DCM (0.5 mL) was added TFA (770 mg, 35 equiv). The reaction was stirred at 0° C. for 1 hours. The mixture was concentrated and diluted with H₂O (5 mL). The mixture was adjusted pH to 8 with saturated Na₂CO₃ aqueous solution (3 mL) and extracted with DCM (2×10 mL), dried over anhydrous sodium sulfate, concentrated and purified with prep-HPLC [Phenomenex Luna C18 150×25 mm×10 um; mobile phase: water (FA)-ACN; B %: 8%-38%, 9 min] to afford the title compound (46.7 mg, 43% yield) as white solid; ¹H NMR (400 MHZ, METHANOL-d₄) δ=9.36 (s, 1H), 8.53 (s, 1H), 7.81 (s, 1H), 4.97-4.92 (m, 1H), 4.84-4.81 (m, 1H), 4.60 (s, 2H), 4.41-4.28 (m, 1H), 3.88-3.72 (m, 1H), 3.67-3.48 (m, 3H), 3.26-3.14 (m, 2H), 2.55 (s, 3H), 2.42 (br s, 1H), 2.35-2.22 (m, 7H), 2.21-2.00 (m, 6H), 1.98-1.90 (m, 1H), 1.87-1.78 (m, 1H), 1.45-1.32 (m, 1H); LCMS (ESI, M+1): m/z=559.4.

### Example 833

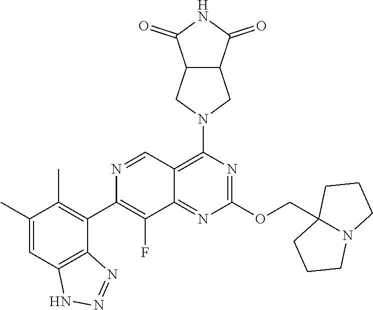 [SMILES: Cc1cc2[nH]nnc2c(-c2ncc3c(N4CC5C(=O)NC(=O)C5C4)nc(OCC45CCCN4CCC5)nc3c2F)c1C]

### 5-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)tetrahydropyrrolo[3,4-c]pyrrole-1,3(2H,3aH)-dione

Synthesized according to Example 832. The title compound was obtained as white solid (FA salt). ¹H NMR (400 MHZ, METHANOL-d₄) δ=9.38 (s, 1H), 7.81 (s, 1H), 4.68-4.63 (m, 4H), 4.40-4.32 (m, 2H), 3.83-3.74 (m, 2H), 3.66-3.60 (m, 2H), 3.27-3.16 (m, 2H), 2.55 (s, 3H), 2.34-2.29 (m, 2H), 2.29-2.27 (s, 3H), 2.24-2.17 (m, 2H), 2.16-2.11 (m, 2H), 2.10-2.03 (m, 2H); LCMS (ESI, M+1): m/z=572.2.

### Example 834

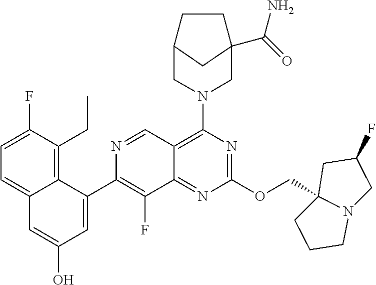 [SMILES: CCc1c(F)ccc2cc(O)cc(-c3ncc4c(N5CC6CCC(C(N)=O)(C6)C5)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxamide

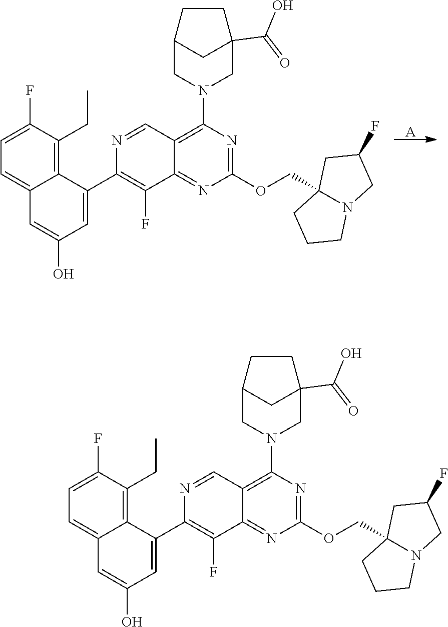

Step A. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxamide: To a solution of 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid (30.0 mg, 1.0 equiv) and NH₄Cl (24.8 mg, 10 equiv) in DMF (0.3 mL) were added EDCI (10.6 mg, 1.2 equiv), HOBt (12.5 mg, 2.0 equiv) and TEA (37.5 mg, 8.0 equiv). The reaction was stirred at 40° C. for 12 hours. The mixture was filtered and purified by prep-HPLC [column: Waters Xbridge 150×25 mm×5 um; mobile phase: A: water (NH₄HCO₃), B: ACN, B %: 40%-70%, 8 min] to afford the title compound (12.9 mg, 42% yield) as white solid; ¹H NMR (400 MHZ, METHANOL-d4) δ=9.11 (d, J=1.2 Hz, 1H), 7.68 (dd, J=5.6, 9.2 Hz, 1H), 7.33-7.20 (m, 2H), 7.06 (t, J=2.4 Hz, 1H), 5.50-5.28 (m, 1H), 5.11-4.99 (m, 1H), 4.68-4.59 (m, 1H), 4.51-4.33 (m, 2H), 3.78-3.47 (m, 3H), 3.46-3.36 (m, 2H), 3.20-3.07 (m, 1H), 2.62-2.30 (m, 4H), 2.28-2.04 (m, 6H), 2.01-1.85 (m, 4H), 1.68-1.52 (m, 1H), 0.84-0.75 (m, 3H); LCMS (ESI, M+1): m/z=647.2.

### Example 835

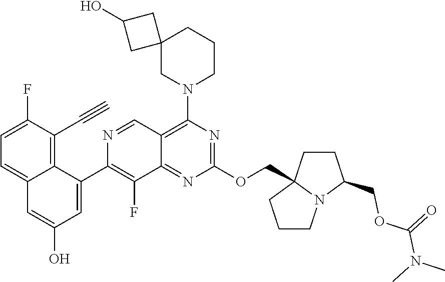 [SMILES: C#Cc1c(F)ccc2cc(O)cc(-c3ncc4c(N5CCCC6(CC(O)C6)C5)nc(OC[C@]56CCCN5[C@H](COC(=O)N(C)C)CC6)nc4c3F)c12]

### ((3S,7aR)-7a-(((7-(8-ethynyl-7-fluoro-3-hydroxynaphthalen-1-yl)-8-fluoro-4-(2-hydroxy-6-azaspiro[3.5]nonan-6-yl)pyrido[4,3-d]pyrimidin-2-yl)oxy)methyl) hexahydro-1H-pyrrolizin-3-yl)methyl dimethylcarbamate

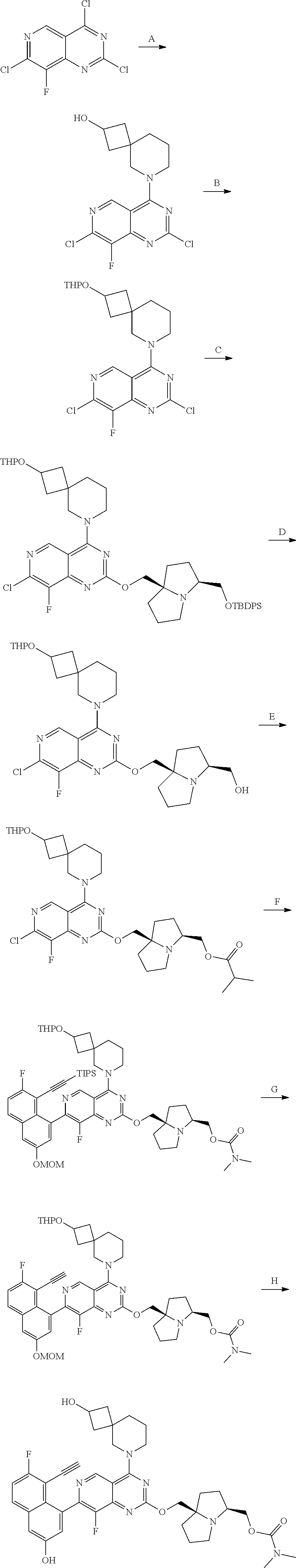

Step A. 6-(2,7-dichloro-8-fluoropyrido[4,3-d]pyrimidin-4-yl)-6-azaspiro[3.5]nonan-2-ol: To a solution of 2,4,7-trichloro-8-fluoropyrido[4,3-d]pyrimidine (1.00 g, 1.0 equiv) and DIEA (1.54 g, 3.0 equiv) in DCM (10 mL) was added 6-azaspiro[3.5]nonan-2-ol (559 mg, 1.0 equiv). The reaction was stirred at −40° C. for 1 hour. The mixture was diluted with water (50 mL), extracted with ethyl acetate (3×50 mL), washed with brine (50 mL), dried, concentrated and purified with column chromatography [SiO₂, petroleum ether/ethyl acetate=5/1 to 2/1] to afford the title compound (800 mg, 56% yield) as a white solid; LCMS (ESI, M+1): m/z=357.1.

Step B. 2,7-dichloro-8-fluoro-4-(2-((tetrahydro-2H-pyran-2-yl)oxy)-6-azaspiro[3.5]nonan-6-yl)pyrido[4,3-d]pyrimidine: To a solution of 6-(2,7-dichloro-8-fluoropyrido[4,3-d]pyrimidin-4-yl)-6-azaspiro[3.5]nonan-2-ol (800 mg, 1.0 equiv) and TsOH (77.1 mg, 0.2 equiv) in DCM (10 mL) was added DHP (282 mg, 1.5 equiv). The reaction was stirred at 20° C. for 16 hours. The mixture was quenched by saturated sodium bicarbonate solution (50 mL) at 0° C., extracted with DCM (50× 3 mL), washed with brine (50 mL), dried, concentrated and purified with prep-TLC [SiO₂, petroleum ether/ethyl acetate=1/1] to afford the title compound (700 mg, 71% yield) as white solid; LCMS (ESI, M+1): m/z=441.1.

Step C. 2-(((3S,7aR)-3-(((tert-butyldiphenylsilyl)oxy)methyl)tetrahydro-1H-pyrrolizin-7a(5H)-yl)methoxy)-7-chloro-8-fluoro-4-(2-((tetrahydro-2H-pyran-2-yl)oxy)-6-azaspiro[3.5]nonan-6-yl)pyrido[4,3-d]pyrimidine: To a solution of ((3S,7aR)-3-(((tert-butyldiphenylsilyl)oxy)methyl) hexahydro-1H-pyrrolizin-7a-yl)methanol (650 mg, 1.0 equiv), 4 Å molecular sieve (50 mg) in toluene (10 mL) was added t-BuONa (2 M, 2.38 mL, 3.0 equiv) at 0° C. for 0.5 hours, and then mixture was added 2,7-dichloro-8-fluoro-4- (2-((tetrahydro-2H-pyran-2-yl)oxy)-6-azaspiro[3.5]nonan-6-yl)pyrido[4,3-d]pyrimidine (700 mg, 1.0 equiv) at 0° C. The reaction was stirred at 0° C. for 0.5 hour under N₂ atmosphere. The mixture was quenched by water (50 mL at 0° C.), extracted with DCM (3× 50 mL), washed with brine (50 mL), dried, concentrated and purified with column chromatography [SiO₂, methylene chloride: methyl alcohol=10:1] to afford the title compound (700 mg, 54% yield) as a yellow solid; LCMS (ESI, M+1): m/z=814.3.

Step D. ((3S,7aR)-7a-(((7-chloro-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methanol: To a solution of t 2-(((3S,7aR)-3-(((tert-butyldiphenylsilyl)oxy)methyl) hexahydro-1H-pyrrolizin-7a-yl)methoxy)-7-chloro-8-fluoro-4-(2-((tetrahydro-2H-pyran-2-yl)oxy)-6-azaspiro[3.5]nonan-6-yl)pyrido[4,3-d]pyrimidine (700 mg, 1.0 equiv) in DMF (8 mL) was added CsF (2.61 g, 20.0 equiv). The reaction was stirred at 40° C. for 12 hours. The mixture was diluted with water (20 mL), extracted with ethyl acetate (3×20 mL), washed with brine (20 mL), dried and concentrated to afford the title compound (300 mg, crude) as a yellow solid; LCMS (ESI, M+1): m/z=576.2.

Step E. ((3S,7aR)-7a-(((7-chloro-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate: To a solution of ((3S,7aR)-7a-(((7-chloro-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methanol (280 mg, 1.0 equiv) and TEA (148 mg, 3.0 equiv) in DCM (5 mL) was added (4-nitrophenyl) carbonochloridate (196 mg, 2.0 equiv) at 0° C. The reaction was stirred at 25° C. for 2 hours, then TEA (98.4 mg, 2.0 equiv) and N-methylmethanamine (2 M, 729 μL, 3.0 equiv) was added at 0° C. The reaction was stirred at 25° C. for 2 hours. The mixture was diluted with water (30 mL), extracted with DCM (3×30 mL), washed with brine (30 mL), dried, concentrated and purified with prep-TLC [SiO₂, methylene dichloride:methyl alcohol=10:1] to afford the title compound (300 mg, 95% yield) as a yellow oil; LCMS (ESI, M+1): m/z=647.4.

Step F. ((3S,7aR)-7a-(((8-fluoro-7-(7-fluoro-3-(methoxymethoxy)-8-((triisopropylsilyl)ethynyl)naphthalen-1-yl)-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate: To a solution of ((3S,7aR)-7a-(((7-chloro-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate (300 mg, 1.0 equiv), Ad₂nBuP Pd G₃ (33.8 mg, 0.1 equiv) and Cs₂CO₃ (453, 3.0 equiv) in water (1 mL) and CPME (5 mL) was added ((2-fluoro-6-(methoxymethoxy)-8-(4,4,5,5-tetramethyl-1,3,2-dioxaborolan-2-yl)naphthalen-1-yl)ethynyl)triisopropylsilane (285 mg, 1.2 equiv), The reaction was stirred at 100° C. for 2 hours under N₂ atmosphere. The mixture was diluted with water (30 mL), extracted with DCM (3×30 mL), washed with brine (30 mL), dried, concentrated and purified with prep-TLC [SiO₂, methylene dichloride:methyl alcohol=10:1] to afford the title compound (200 mg, 43% yield) as yellow solid;

Step G. ((3S,7aR)-7a-(((7-(8-ethynyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate: To a solution of ((3S,7aR)-7a-(((8-fluoro-7-(7-fluoro-3-(methoxymethoxy)-8-((triisopropylsilyl)ethynyl)naphthalen-1-yl)-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate (50.0 mg, 1.0 equiv) in DMF (2 mL) was added CsF (228 mg, 30 equiv). The mixture was stirred at 20° C. for 2 hours. The reaction mixture was diluted with water (20 mL), extracted with DCM (3×20 mL), washed with brine (20 mL), dried, and concentrated to afford the title compound (50 mg, crude) as a yellow oil; LCMS (ESI, M+1): m/z=841.4.

Step H. ((3S,7aR)-7a-(((7-(8-ethynyl-7-fluoro-3-hydroxynaphthalen-1-yl)-8-fluoro-4-(2-hydroxy-6-azaspiro[3.5]nonan-6-yl)pyrido[4,3-d]pyrimidin-2-yl)oxy)methyl) hexahydro-1H-pyrrolizin-3-yl)methyl dimethylcarbamate: To a solution of ((3S,7aR)-7a-(((7-(8-ethynyl-7-fluoro-3-(methoxymethoxy)naphthalen-1-yl)-8-fluoro-4-(2-((tetrahydro-2H-pyran-2-yl)oxy)-6-azaspiro[3.5]nonan-6-yl)pyrido[4,3-d]pyrimidin-2-yl)oxy)methyl) hexahydro-1H-pyrrolizin-3-yl)methyl dimethylcarbamate (60.0 mg, 1.0 equiv) in ACN (2 mL) was added HCl·dioxane (4 M, 2 mL). The reaction was stirred at 0° C. for 0.5 hours. The reaction mixture was quenched by NaHCO₃, adjusted pH to 7 at 0° C., diluted with water (20 mL), extracted with methylene dichloride (3×20 mL), washed with brine (20 mL), dried, concentrated and purified with prep-HPLC [column: Waters Xbridge 150×25 mm×5 um; mobile phase: [water (NH₄HCO₃)-ACN]; B %: 38%-68%, 8 min] to afford the title compound (7.80 mg, 15% yield) as yellow solid; ¹H NMR (400 MHZ, METHANOL-d₄) δ=8.99 (d, J=7.2 Hz, 1H), 7.86 (dd, J=5.6, 9.2 Hz, 1H), 7.37-7.29 (m, 2H), 7.25-7.20 (m, 1H), 4.68-4.55 (m, 1H), 4.38-4.16 (m, 3H), 4.15-3.86 (m, 6H), 3.51-3.37 (m, 1H), 3.04 (td, J=5.2, 10.0 Hz, 2H), 2.92 (br d, J=18.4 Hz, 6H), 2.83 (br dd, J=5.2, 10.4 Hz, 1H), 2.41-2.27 (m, 1H), 2.25-2.18 (m, 2H), 2.07-1.99 (m, 1H), 1.97-1.87 (m, 3H), 1.82 (br s, 5H), 1.71 (br dd, J=7.2, 11.6 Hz, 3H); LCMS (ESI, M+1): m/z=713.3.

### Example 836

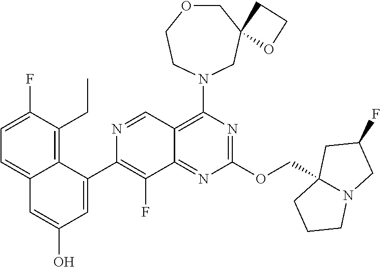 [SMILES: CCc1c(F)ccc2cc(O)cc(-c3ncc4c(N5CCOC[C@@]6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((R)-1,6-dioxa-9-azaspiro[3.6]decan-9-yl)pyrido[4,3-d]pyrimidin-7-yl)naphthalen-2-ol

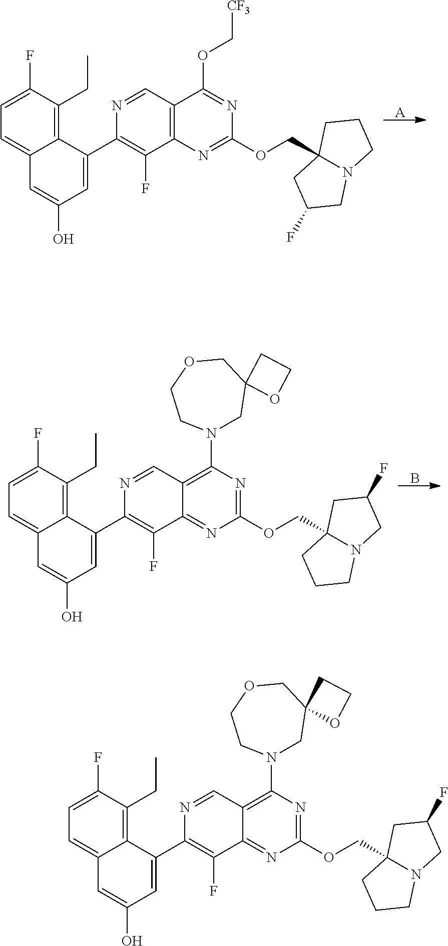

Step A. 5-ethyl-6-fluoro-4-(8-fluoro-2-(((2R,7aS)-2-fluorohexahydro-1H-pyrrolizin-7a-yl)methoxy)-4-(1,6-dioxa-9-azaspiro[3.6]decan-9-yl)pyrido[4,3-d]pyrimidin-7-yl)naphthalen-2-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (150 mg, 1.0 equiv) and 1,6-dioxa-9-azaspiro[3.6]decane (109 mg, 3.0 equiv) in DMF (1 mL) were added 4 Å molecular sieve (10.0 mg, 1.0 equiv) and K₃PO₄ (537 mg, 10 equiv). The reaction was stirred at 60° C. for 2 hours. The mixture was filtered and purified with prep-HPLC [column: Waters Xbridge 150×25 mm×5 um; mobile phase: [water (ammonia hydroxide v/v)-ACN]; B %: 30%-60%, 9 min] to afford the title compound (41.6 mg, 29% yield) as off-white solid; LCMS (ESI, M+1): m/z=636.3.

Step B. 5-ethyl-6-fluoro-4-(8-fluoro-2-(((2R,7aS)-2-fluorotetrahydro-1H-pyrrolizin-7a(5H)-yl)methoxy)-4-((R)-1,6-dioxa-9-azaspiro[3.6]decan-9-yl)pyrido[4,3-d]pyrimidin-7-yl)naphthalen-2-ol: 5-ethyl-6-fluoro-4-(8-fluoro-2-(((2R,7aS)-2-fluorotetrahydro-1H-pyrrolizin-7a(5H)-yl)methoxy)-4-(1,6-dioxa-9-azaspiro[3.6]decan-9-yl)pyrido[4,3-d]pyrimidin-7-yl)naphthalen-2-ol (41.6 mg) was separated by SFC [Column: Cellulose-4 50×4.6 mm I.D., 3 um Mobile phase: Phase A for CO₂, and Phase B for EtOH (0.05% DEA); Gradient elution: 40% EtOH (0.05% DEA) in CO₂ Flow rate: 3 mL/min; Detector: PDA Column Temp: 35C; Back Pressure: 100 Bar] to afford the title compound (6.44 mg, 4% yield) as a white solid; ¹H NMR (400 MHZ, METHANOL-d4) δ=9.54 (s, 1H), 7.68 (dd, J=5.8, 9.2 Hz, 1H), 7.31 (d, J=2.4 Hz, 1H), 7.25 (t, J=9.6 Hz, 1H), 7.06 (dd, J=2.5, 10.4 Hz, 1H), 5.43-5.20 (m, 1H), 4.69-4.53 (m, 4H), 4.40-4.32 (m, 1H), 4.30-4.16 (m, 2H), 4.13-3.95 (m, 4H), 3.92-3.80 (m, 1H), 3.29-3.17 (m, 3H), 3.03 (dt, J=5.6, 9.2 Hz, 1H), 2.77-2.65 (m, 1H), 2.62-2.43 (m, 2H), 2.39-2.20 (m, 2H), 2.20-2.11 (m, 2H), 2.05-1.88 (m, 3H), 0.80 (td, J=7.2, 17.2 Hz, 3H); LCMS (ESI, M+1): m/z=636.5.

### Example 837

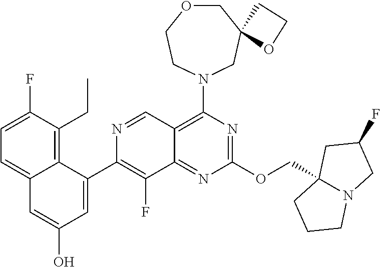 [SMILES: CCc1c(F)ccc2cc(O)cc(-c3ncc4c(N5CCOC[C@]6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((S)-1,6-dioxa-9-azaspiro[3.6]decan-9-yl)pyrido[4,3-d]pyrimidin-7-yl)naphthalen-2-ol

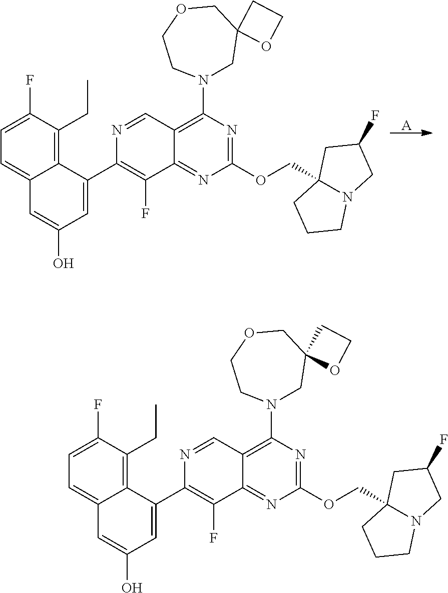

Step A. 5-ethyl-6-fluoro-4-(8-fluoro-2-(((2R,7aS)-2-fluorotetrahydro-1H-pyrrolizin-7a(5H)-yl)methoxy)-4-((S)-1,6-dioxa-9-azaspiro[3.6]decan-9-yl)pyrido[4,3-d]pyrimidin-7-yl)naphthalen-2-ol: 5-ethyl-6-fluoro-4-(8-fluoro-2-(((2R,7aS)-2-fluorotetrahydro-1H-pyrrolizin-7a(5H)-yl)methoxy)-4-(1,6-dioxa-9-azaspiro[3.6]decan-9-yl)pyrido[4,3-d]pyrimidin-7-yl)naphthalen-2-ol (41.6 mg) was separated by SFC [Column: Cellulose-4 50×4.6 mm I.D., 3 um Mobile phase: Phase A for CO₂, and Phase B for EtOH (0.05% DEA); Gradient elution: 40% EtOH (0.05% DEA) in CO₂ Flow rate: 3 mL/min; Detector: PDA Column Temp: 35C; Back Pressure: 100 Bar] to afford the title compound (5.70 mg, 3% yield) as a yellow solid; ¹H NMR (400 MHZ, METHANOL-d4) δ=9.62 (s, 1H), 7.69 (dd, J=6.0, 9.2 Hz, 1H), 7.32 (d, J=2.4 Hz, 1H), 7.26 (t, J=9.6 Hz, 1H), 7.09-7.03 (m, 1H), 5.65-5.40 (m, 1H), 4.73-4.52 (m, 6H), 4.27-4.14 (m, 1H), 4.13-3.99 (m, 4H), 3.94-3.71 (m, 4H), 3.38 (br dd, J=4.0, 9.6 Hz, 1H), 2.78-2.67 (m, 1H), 2.66-2.43 (m, 4H), 2.42-2.34 (m, 1H), 2.33-2.23 (m, 2H), 2.22-2.07 (m, 2H), 0.86-0.74 (m, 3H); LCMS (ESI, M+1): m/z=636.5.

### Example 838

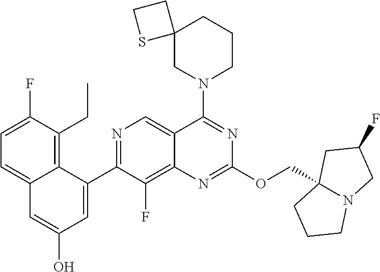 [SMILES: CCc1c(F)ccc2cc(O)cc(-c3ncc4c(N5CCCC6(CCS6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-thia-6-azaspiro[3.5]nonan-6-yl)pyrido[4,3-d]pyrimidin-7-yl)naphthalen-2-ol

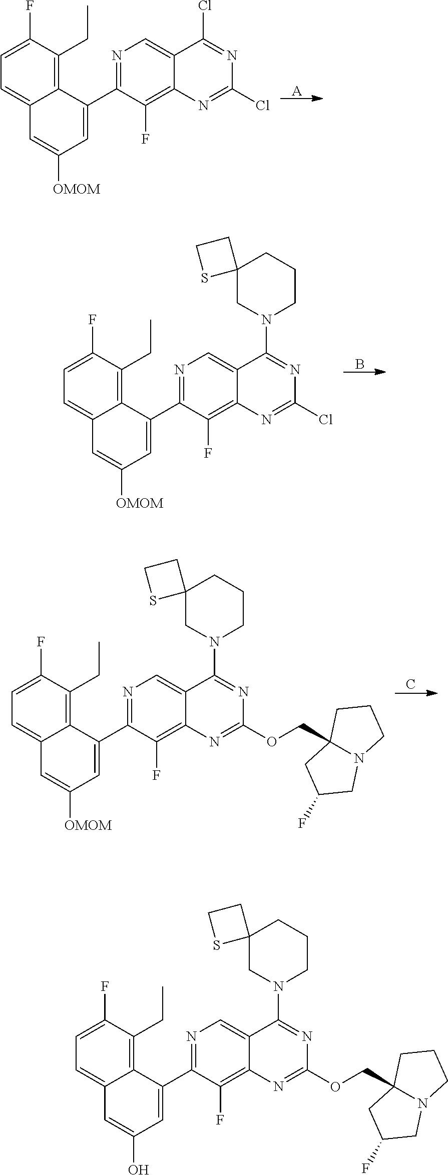

Step A. 6-(2-chloro-7-(8-ethyl-7-fluoro-3-(methoxymethoxy)naphthalen-1-yl)-8-fluoropyrido[4,3-d]pyrimidin-4-yl)-1-thia-6-azaspiro[3.5]nonane: To a mixture of 2,4-dichloro-7-(8-ethyl-7-fluoro-3-(methoxymethoxy)naphthalen-1-yl)-8-fluoropyrido[4,3-d]pyrimidine (66.0 mg, 1.0 equiv) and 1-thia-6-azaspiro[3.5]nonane (23.7 mg, 0.9 equiv, HCl salt) in DCM (0.6 mL) were added DIEA (75.8 mg, 4.0 equiv) and 4 Å molecular sieve (50 mg). The reaction was stirred at 0° C. for 30 minutes. The mixture was quenched with water (5 mL) and extracted with DCM (2×5 mL). The combined organic layers were dried over anhydrous sodium sulfate, concentrated and purified with column chromatography (SiO₂, Petroleum ether/Ethyl acetate=2:1) to afford the title compound (32.0 mg, 39% yield) as yellow solid. ¹H NMR (400 MHZ, CHLOROFORM-d) δ=9.47 (d, J=6.4 Hz, 1H), 7.70 (dd, J=5.6, 8.8 Hz, 1H), 7.55 (d, J=2.4 Hz, 1H), 7.29 (s, 1H), 7.24 (d, J=2.4 Hz, 1H), 5.36-5.25 (m, 2H), 4.75-4.50 (m, 1H), 4.41-4.22 (m, 1H), 4.02-3.82 (m, 1H), 3.52 (d, J=0.8 Hz, 3H), 3.50-3.38 (m, 1H), 3.24-3.05 (m, 2H), 2.82-2.63 (m, 2H), 2.59-2.44 (m, 1H), 2.38-2.26 (m, 1H), 2.23-2.11 (m, 1H), 2.04-1.99 (m, 1H), 1.89 (br d, J=4.4 Hz, 2H), 0.85 (q, J=7.2 Hz, 3H); LCMS (ESI, M+1): m/z=557.1.

Step B. 6-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-1-thia-6-azaspiro[3.5]nonane: To a mixture of 6-(2-chloro-7-(8-ethyl-7-fluoro-3-(methoxymethoxy)naphthalen-1-yl)-8-fluoropyrido[4,3-d]pyrimidin-4-yl)-1-thia-6-azaspiro[3.5]nonane (50.0 mg, 1.0 equiv) and 4 Å molecular sieve (50 mg, 1.0 equiv) in toluene (0.5 mL) was added t-BuONa (25.9 mg, 3.0 equiv). The reaction was stirred at 0° C. for 10 minutes. To the mixture was added ((2R,7aS)-2-fluorotetrahydro-1H-pyrrolizin-7a(5H)-yl)methanol (15.7 mg, 1.1 equiv). The reaction was stirred at 0° C. for 1 hour. The mixture was quenched with H₂O (10 mL) at 0° C. and extracted with ethyl acetate (3×10 mL). The combined organic layers were dried over anhydrous sodium sulfate, concentrated and purified with prep-HPLC (column: Phenomenex Luna C18 150*25 mm*10 um; mobile phase: [water (FA)-ACN]; B %: 24%-57%, 9 min) to afford the title compound (18.0 mg, 30% yield) as white solid; LCMS (ESI, M+1): m/z=680.3.

Step C. 5-ethyl-6-fluoro-4-(8-fluoro-2-(((2R,7aS)-2-fluorohexahydro-1H-pyrrolizin-7a-yl)methoxy)-4-(1-thia-6-azaspiro[3.5]nonan-6-yl)pyrido[4,3-d]pyrimidin-7-yl)naphthalen-2-ol: To a solution of 6-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-1-thia-6-azaspiro[3.5]nonane (21.0 mg, 1.0 equiv) in DCM (1 mL) was added TFA (500 uL). The reaction was stirred at 0° C. for 2 hours. The mixture was concentrated under vacuum. The residue was diluted with water (1 mL). The mixture was adjusted to pH=7 with saturated NaHCO₃ (3 mL) and extracted with ethyl acetate (3× 3 mL). The combined organic layers were dried over anhydrous sodium sulfate, concentrated and purified with prep-HPLC (column: Phenomenex luna C18 150*25 mm*10 um; mobile phase: [water (FA)-ACN]; B %: 24%-44%, 10 min) to afford the title compound (6.00 mg, 30% yield) as yellow solid. ¹H NMR (400 MHZ, METHANOL-d₄) δ=9.44-9.28 (m, 1H), 8.53 (br d, J=2.8 Hz, 1H), 7.68 (dd, J=6.0, 8.8 Hz, 1H), 7.40-7.19 (m, 2H), 7.10 (br d, J=2.4 Hz, 1H), 5.56-5.24 (m, 1H), 4.64 (br d, J=13.2 Hz, 1H), 4.51-4.27 (m, 3H), 4.05-3.88 (m, 1H), 3.58-3.37 (m, 4H), 3.21-2.99 (m, 3H), 2.82-2.63 (m, 2H), 2.54-2.32 (m, 3H), 2.31-1.94 (m, 7H), 1.82 (br d, J=3.6 Hz, 2H), 0.80 (q, J=7.2 Hz, 3H); LCMS (ESI, M+1): m/z=636.3.

### Example 839

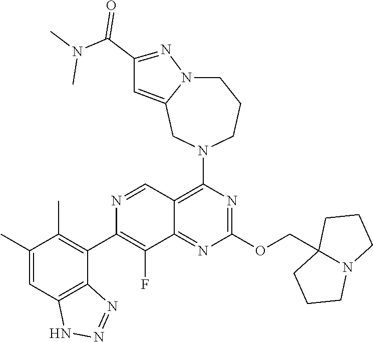 [SMILES: Cc1cc2[nH]nnc2c(-c2ncc3c(N4CCCn5nc(C(=O)N(C)C)cc5C4)nc(OCC45CCCN4CCC5)nc3c2F)c1C]

### 5-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

Synthesized according to Example 832. The title compound was obtained as white solid (FA salt). ¹H NMR (400 MHz, METHANOL-d₄) δ=9.37-9.23 (m, 1H), 8.57-8.52 (m, 1H), 7.93-7.71 (m, 1H), 6.86-6.76 (m, 1H), 5.32-5.24 (m, 2H), 4.57 (s, 2H), 4.56-4.46 (m, 4H), 3.64-3.51 (m, 2H), 3.37-3.34 (m, 3H), 3.22-3.14 (m, 2H), 3.12-3.05 (m, 3H), 2.58-2.52 (m, 3H), 2.51-2.41 (m, 2H), 2.34-2.23 (m, 5H), 2.22-1.99 (m, 6H); LCMS (ESI, M+1): m/z=640.4.

### Example 840

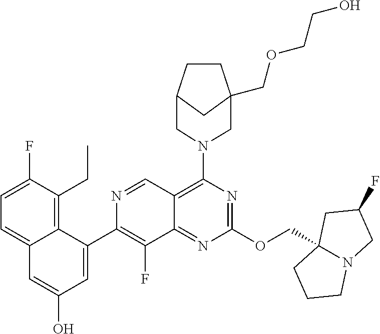 [SMILES: CCc1c(F)ccc2cc(O)cc(-c3ncc4c(N5CC6CCC(COCCO)(C6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-((2-hydroxyethoxy)methyl)-3-azabicyclo[3.2.1]octan-3-yl)pyrido[4,3-d]pyrimidin-7-yl)naphthalen-2-ol

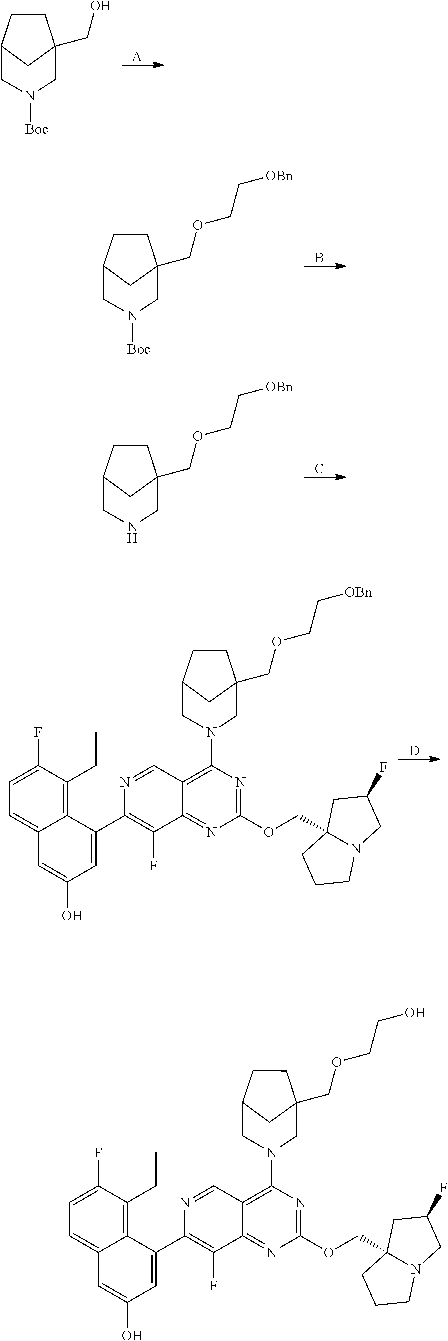

Step A. tert-butyl 1-((2-(benzyloxy) ethoxy)methyl)-3-azabicyclo[3.2.1]octane-3-carboxylate: To a solution of tert-butyl 1-(hydroxymethyl)-3-azabicyclo[3.2.1]octane-3-carboxylate (300 mg, 1.0 equiv) in DMAC (3.00 mL) was added NaH (149 mg, 60% purity, 3.0 equiv) at 0° C. The mixture was stirred at 0° C. for 0.5 hours. Then ((2-iodoethoxy)methyl)benzene (977 mg, 3.0 equiv) was added. The reaction was stirred at 25° C. for 2 hours. The mixture was quenched with water (20 mL) and extracted with ethyl acetate (2×10 mL). The combined organic layers were dried over anhydrous sodium sulfate, concentrated, and purified with reversed phase flash chromatography [C18, 0.1% formic acid condition] to afford the title compound (137 mg, 28% yield) as yellow oil; ¹H NMR (400 MHZ, CHLOROFORM-d) δ=7.38-7.28 (m, 5H), 4.58 (br s, 2H), 3.89 (br d, J=11.2 Hz, 1H), 3.82-3.70 (m, 1H), 3.62 (br d, J=4.4 Hz, 4H), 3.42-3.35 (m, 2H), 2.88-2.69 (m, 2H), 2.31-2.16 (m, 1H), 1.77-1.63 (m, 2H), 1.63-1.49 (m, 3H), 1.46 (s, 9H), 1.43-1.38 (m, 1H); LCMS (ESI, M−55): m/z=320.2.

Step B. 1-((2-(benzyloxy) ethoxy)methyl)-3-azabicyclo[3.2.1]octane: To a solution of tert-butyl 1-((2-(benzyloxy) ethoxy)methyl)-3-azabicyclo[3.2.1]octane-3-carboxylate (130 mg, 1.0 equiv) in acetonitrile (1.00 mL) was added HCl·dioxane (4 M, 2.00 mL). The reaction mixture was stirred at 20° C. for 1 hour. The mixture was concentrated under vacuum to afford the title compound (110 mg, crude, HCl salt) as yellow oil; LCMS (ESI, M+1): m/z=276.3.

Step C. 4-(4-(1-((2-(benzyloxy) ethoxy)methyl)-3-azabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (150 mg, 1.0 equiv), DIEA (164 mg, 5.0 equiv), and 4 Å molecular sieve (30.0 mg) in DMF (1.50 mL) 1-((2-(benzyloxy) ethoxy)methyl)-3-azabicyclo[3.2.1]octane (103 mg, 1.3 equiv, HCl). The reaction mixture was stirred at 40° C. for 16 hours. The mixture was filtered and the filtrate was purified with reversed phase flash chromatography [C18, 0.1% formic acid condition] to afford the title compound (103 mg, 50% yield) as yellow solid; LCMS (ESI, M+1): m/z=768.4.

Step D. 5-ethyl-6-fluoro-4-(8-fluoro-2-(((2R,7aS)-2-fluorohexahydro-1H-pyrrolizin-7a-yl)methoxy)-4-(1-((2-hydroxyethoxy)methyl)-3-azabicyclo[3.2.1]octan-3-yl)pyrido[4,3-d]pyrimidin-7-yl)naphthalen-2-ol: To a solution of 4-(4-(1-((2-(benzyloxy) ethoxy)methyl)-3-azabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol (80 mg, 1.0 equiv) in MeOH (5.00 mL) was added Pd(OH)₂/C (50.0 mg, 20% purity) under N₂ atmosphere. The suspension was degassed and purged with H₂ for 3 times. The reaction was stirred at 20° C. for 5 hours under H₂ (15 Psi). The mixture was filtered and the filtrate was concentrated under vacuum to give a residue. The residue was purified with prep-HPLC [Waters Xbridge 150×25 mm×5 μm: A: water (NH₄HCO₃), B: CAN, B %: 48%-78% over 8 min] to afford the title compound (5.52 mg, 8.0% yield) as yellow solid; ¹H NMR (400 MHz, METHANOL-d4) δ=9.09 (d, 1H), 7.73-7.62 (m, 1H), 7.34-7.20 (m, 2H), 7.07 (br s, 1H), 5.42-5.25 (m, 1H), 4.78-4.61 (m, 2H), 4.41-4.20 (m, 2H), 3.75-3.61 (m, 3H), 3.59-3.47 (m, 5H), 3.29-3.18 (m, 2H), 3.16-2.99 (m, 1H), 2.55-2.40 (m, 2H), 2.39-2.23 (m, 2H), 2.22-2.12 (m, 2H), 2.08-1.97 (m, 2H), 1.97-1.43 (m, 8H), 0.87-0.75 (m, 3H); LCMS (ESI, M+1): m/z=678.5.

### Example 841

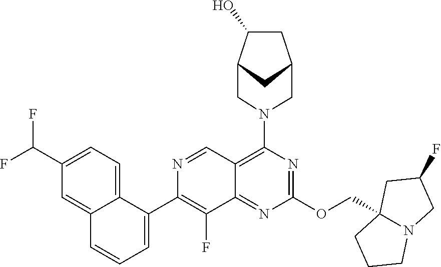 [SMILES: O[C@@H]1C[C@H]2C[C@@H]1CN(c1nc(OC[C@@]34CCCN3C[C@H](F)C4)nc3c(F)c(-c4cccc5cc(C(F)F)ccc45)ncc13)C2]

### (1R,5R,6R)-3-(7-(6-(difluoromethyl)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

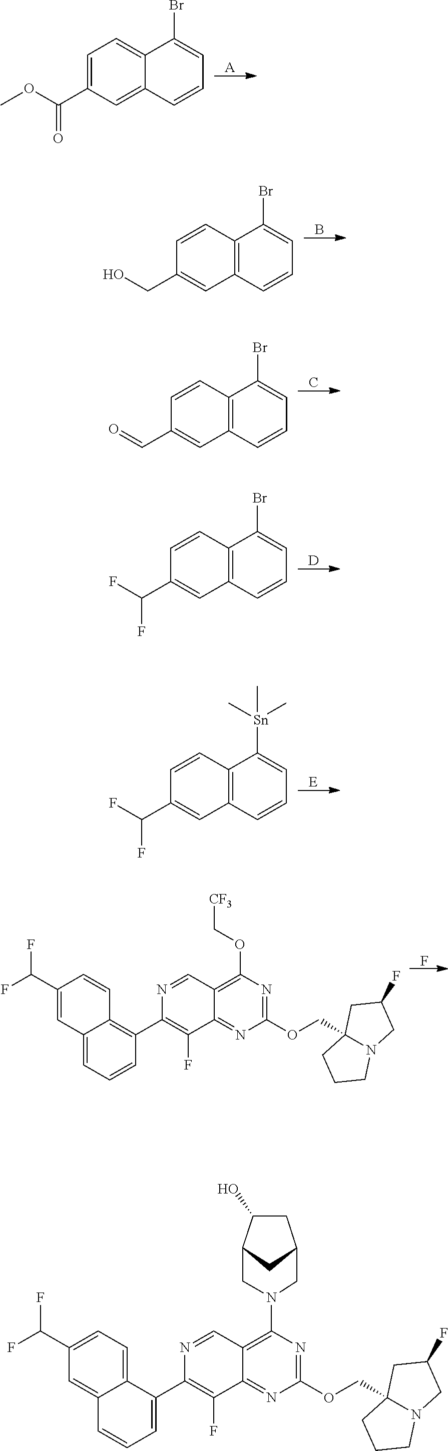

Step A. (5-bromonaphthalen-2-yl)methanol: To a solution of methyl 5-bromo-2-naphthoate (100 mg, 1.0 equiv) in THF (2 mL) was added LAH (28.6 mg, 2.0 equiv) at −40° C. in portions. The reaction was stirred at −40° C. for 1 hour. The mixture was quenched with H₂O (0.2 mL), 15% NaOH solution (0.2 mL), H₂O (0.6 mL), The residue was filtered and the filtrate was concentrated under vacuum to afford the title compound (50.0 mg, crude) as white solid.

Step B: 5-bromo-2-naphthaldehyde: To a solution of (5-bromonaphthalen-2-yl)methanol (1.16 g, 1.0 equiv) in dichloromethane (20 mL) was added Dess-Martin (2.49 g, 1.2 equiv) at 25° C. The reaction was stirred at 25° C. for 1 hour. The mixture was quenched with saturated Na₂SO₃ (15 mL) and extracted with dichloromethane (20 mL). The organic layer was dried over sodium sulfate, concentrated and purified by column chromatography (SiO₂, petroleum ether/ethyl acetate=50/1 to 10/1) to afford the title compound (476 mg, 41% yield) as a yellow solid. ¹H NMR (400 MHZ, DMSO-d6) δ=10.20 (s, 1H), 8.67 (s, 1H), 8.26 (dd, J=8.8, 15.2 Hz, 2H), 8.10-8.03 (m, 2H), 7.59 (t, J=8.0 Hz, 1H).

Step C: 1-bromo-6-(difluoromethyl)naphthalene: A mixture of 5-bromo-2-naphthaldehyde (470 mg, 1.0 equiv) and BAST (6.41 g, 14 equiv) was stirred at 25° C. for 1 hour. The reaction was diluted with water (6 mL) and extracted with ethyl acetate (8 mL). The organic layer was dried over sodium sulfate, concentrated and purified by column chromatography (SiO₂, petroleum ether/ethyl acetate=50/1 to 10/1) to afford the title compound (370 mg, 72% yield) as a yellow oil. ¹H NMR (400 MHZ, DMSO-d6) δ=8.29-8.22 (m, 2H), 8.12 (d, J=8.0 Hz, 1H), 7.99 (d, J=7.6 Hz, 1H), 7.82 (d, J=8.8 Hz, 1H), 7.54 (t, J=7.6 Hz, 1H), 7.25 (t, J=55.6 Hz, 1H).

Step D: (6-(difluoromethyl)naphthalen-1-yl)trimethylstannane: A mixture of 1-bromo-6-(difluoromethyl)naphthalene (311 mg, 1.0 equiv), trimethyl(trimethylstannyl) stannane (3.96 g, 10 equiv) and Pd(PPh₃)₄ (279 mg, 0.2 equiv) in toluene (20 mL) was degassed under vacuum and purged with N₂ three times. The reaction was stirred at 100° C. for 14 hours. The mixture was diluted with water (60 mL) and extracted with ethyl acetate (100 mL). The organic layer was dried over sodium sulfate, concentrated and purified by column chromatography (SiO₂, petroleum ether) to afford the title compound (380 mg, 92% yield) as colourless oil.

Step E 7-(6-(difluoromethyl)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (140 mg, 1.0 equiv), [6-(difluoromethyl)-1-naphthyl]-trimethyl-stannane (217 mg, 2.0 equiv), BINAP (39.7 mg, 0.2 equiv), CuI (24.3 mg, 0.4 equiv) in toluene (10 mL) was added Pd(dppf)Cl₂ (23.3 mg, 0.1 equiv). The mixture was degassed and purged with N₂ for 3 times. The reaction was stirred at 100° C. for 16 hours under N₂ atmosphere. The mixture was diluted with water (15 mL) and extracted with ethyl acetate (15 mL). The organic layer was dried over sodium sulfate, concentrated and purified by reversed-phase HPLC (0.1% FA condition) to afford the title compound (49.0 mg, 20% yield) as a brown solid. LCMS (ESI, M+1): m/z=581.3.

Step F (1R,5R,6R)-3-(7-(6-(difluoromethyl)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(6-(difluoromethyl)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (49 mg, 1.0 equiv) and (1R,5R,6R)-3-azabicyclo[3.2.1]octan-6-ol (21.5 mg, 2.0 equiv) in DMF (2.0 mL) was added DIEA (32.7 mg, 3.0 equiv) and 4 Å molecular sieve (40.0 mg) at 40° C. The mixture was warmed to 40° C. and stirred for 14 hours. The mixture was diluted with water (3 mL) and extracted with ethyl acetate (5 mL).

The organic layer was dried over sodium sulfate, filtered and concentrated and purified by prep-HPLC [column: Waters Xbridge 150×50 mm×10 μm; A: water (NH₄HCO₃), B: ACN; B %: 50%-80% over 10 min] to afford the title compound (9.20 mg, 18% yield) as a white solid. ¹H NMR (400 MHZ, METHANOL-d4) δ=9.33 (s, 1H), 8.22-8.14 (m, 2H), 7.83 (br d, J=9.2 Hz, 1H), 7.79-7.71 (m, 2H), 7.62 (d, J=8.8 Hz, 1H), 6.95 (t, J=56.8 Hz, 1H), 5.52-5.29 (m, 1H), 4.97 (br s, 1H), 4.51-4.42 (m, 1H), 4.40-4.30 (m, 2H), 3.80 (br d, J=12.4 Hz, 1H), 3.60-3.40 (m, 4H), 3.22-3.08 (m, 1H), 2.52-2.32 (m, 3H), 2.30-2.19 (m, 3H), 2.17-2.06 (m, 2H), 2.05-1.89 (m, 2H), 1.87-1.77 (m, 1H), 1.45-1.35 (m, 1H); LCMS (ESI, M+1): m/z=608.4.

### Example 842

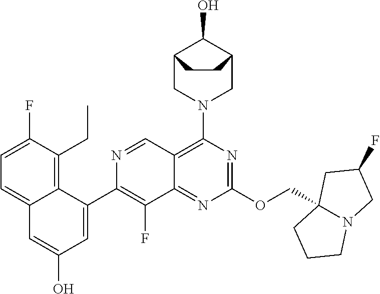 [SMILES: CCc1c(F)ccc2cc(O)cc(-c3ncc4c(N5C[C@H]6CC[C@@H](C5)[C@@H]6O)nc(OC[C@@]56CCCN5C[C@H](F)C6)nc4c3F)c12]

### (1R,5S,8R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-ol

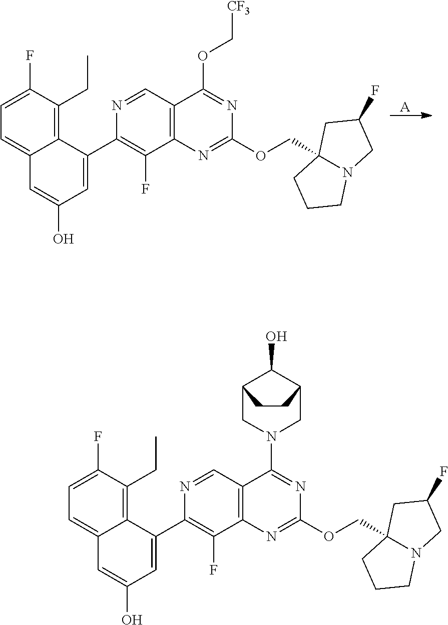

Step A. (1R,5S,8R)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-ol: The mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) and (1R,5S,8s)-3-azabicyclo[3.2.1]octan-8-ol (21.5 mg, 1.5 equiv, HCl) in DMF (0.5 mL) were added DIEA (109 mg, 10 equiv) and 4 Å molecular sieve (10.0 mg, 1.0 equiv). The reaction was stirred at 60° C. for 12 hours. The mixture was filtered and purified by prep-HPLC (column: Waters xbridge 150*25 mm 10 um; mobile phase: [water (NH₄HCO₃)-ACN]; B %: 30%-60%, 11 min) to afford the title compound (11.8 mg, 22% yield) as yellow solid. ¹H NMR (400 MHZ, METHANOL-d4) δ=9.04 (s, 1H), 7.67 (dd, J=5.6, 8.8 Hz, 1H), 7.37-7.16 (m, 2H), 7.06 (d, J=2.4 Hz, 1H), 5.48-5.18 (m, 1H), 4.74-4.68 (m, 2H), 4.36-4.12 (m, 3H), 3.64 (br t, J=9.6 Hz, 2H), 3.26-3.11 (m, 3H), 3.07-2.94 (m, 1H), 2.48 (br s, 1H), 2.40-2.07 (m, 6H), 2.05-1.82 (m, 5H), 1.53 (br d, J=9.6 Hz, 2H), 0.87-0.71 (m, 3H); LCMS (ESI, M+1): m/z=620.3.

### Example 843

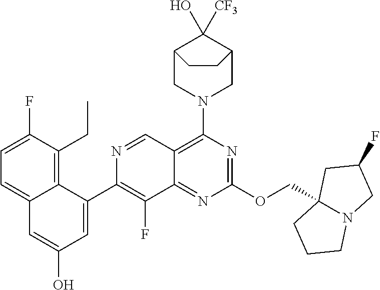 [SMILES: CCc1c(F)ccc2cc(O)cc(-c3ncc4c(N5CC6CCC(C5)C6(O)C(F)(F)F)nc(OC[C@@]56CCCN5C[C@H](F)C6)nc4c3F)c12]

### (1R,5S,8R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-(trifluoromethyl)-3-azabicyclo[3.2.1]octan-8-ol

Synthesized according to Example 842. The title compound was obtained as white solid. ¹H NMR (400 MHZ, METHANOL-d₄) δ=9.94 (br s, 1H), 9.15 (s, 1H), 7.76 (dd, J=6.4, 8.8 Hz, 1H), 7.42-7.26 (m, 2H), 7.02 (d, J=2.0 Hz, 1H), 6.85 (s, 1H), 5.41-5.12 (m, 1H), 4.50 (br t, J=13.6 Hz, 2H), 4.23-3.93 (m, 4H), 3.14-2.97 (m, 3H), 2.89-2.77 (m, 1H), 2.45-2.30 (m, 3H), 2.19-1.98 (m, 4H), 1.91-1.72 (m, 5H), 1.63 (br d, J=8.0 Hz, 2H), 0.72 (t, J=7.2 Hz, 3H); LCMS (ESI, M+1): m/z=688.0.

### Example 844

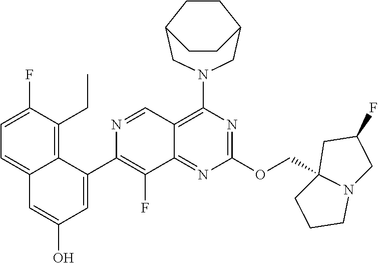 [SMILES: CCc1c(F)ccc2cc(O)cc(-c3ncc4c(N5CC6CCC(CC6)C5)nc(OC[C@@]56CCCN5C[C@H](F)C6)nc4c3F)c12]

### 4-(4-(3-azabicyclo[3.2.2]nonan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

Synthesized according to Example 842. The title compound was obtained as orange solid; ¹H NMR (400 MHz, METHANOL-d₄) δ=9.05 (s, 1H), 7.74-7.61 (m, 1H), 7.37-7.19 (m, 2H), 7.06 (d, J=2.4 Hz, 1H), 5.44-5.19 (m, 1H), 4.36-4.31 (m, 1H), 4.30-4.17 (m, 4H), 3.27-3.12 (m, 3H), 3.06-2.96 (m, 1H), 2.55-2.42 (m, 1H), 2.39-2.08 (m, 6H), 2.06-1.94 (m, 2H), 1.93-1.73 (m, 9H), 0.94-0.68 (m, 3H); LCMS (ESI, M+1): m/z=618.4.

### Example 845

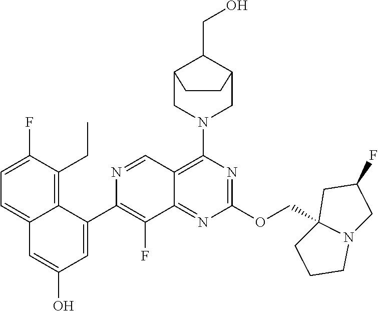 [SMILES: CCc1c(F)ccc2cc(O)cc(-c3ncc4c(N5CC6CCC(C5)C6CO)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5S,8S)-8-(hydroxymethyl)-3-azabicyclo[3.2.1]octan-3-yl)pyrido[4,3-d]pyrimidin-7-yl)naphthalen-2-ol

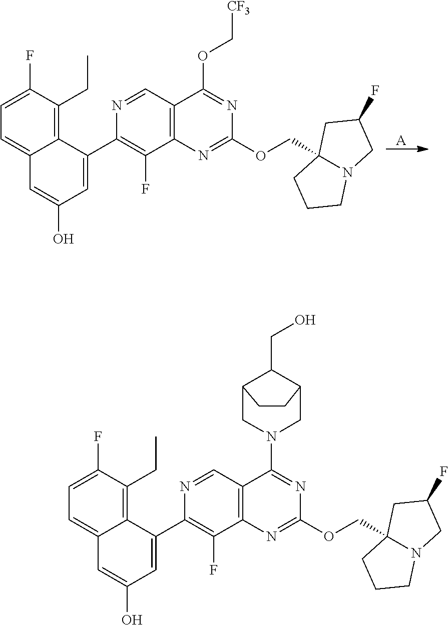

Synthesized according to Example 842. The title compound was obtained as yellow solid (FA salt). ¹H NMR (400 MHZ, METHANOL-d4) δ=9.09 (s, 1H), 8.51 (s, 1H), 7.68 (dd, J=6.0, 9.2 Hz, 1H), 7.34-7.20 (m, 2H), 7.06 (d, J=2.4 Hz, 1H), 5.51-5.27 (m, 1H), 4.78 (br d, J=12 Hz, 2H), 4.49-4.41 (m, 1H), 4.40-4.33 (m, 1H), 3.69-3.58 (m, 2H), 3.56-3.36 (m, 5H), 3.17 (br d, J=5.2 Hz, 1H), 2.34 (br s, 5H), 2.28-2.05 (m, 5H), 2.02-1.91 (m, 1H), 1.87-1.73 (m, 2H), 1.55 (br d, J=10.4 Hz, 2H), 0.79 (dt, J=2.4, 7.2 Hz, 3H). LCMS (ESI, M+1): m/z=634.3

### Example 846

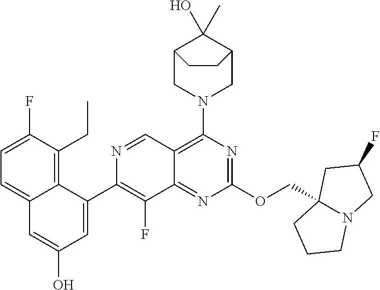 [SMILES: CCc1c(F)ccc2cc(O)cc(-c3ncc4c(N5CC6CCC(C5)C6(C)O)nc(OC[C@@]56CCCN5C[C@H](F)C6)nc4c3F)c12]

### (1R,5S,8R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8-methyl-3-azabicyclo[3.2.1]octan-8-ol

Synthesized according to Example 842. The title compound was obtained as yellow solid. ¹H NMR (400 MHZ, DMSO-d₆) δ=10.16-9.69 (m, 1H), 9.15 (s, 1H), 7.76 (dd, J=6.0, 9.2 Hz, 1H), 7.39-7.30 (m, 2H), 7.02 (d, J=2.0 Hz, 1H), 5.36-5.18 (m, 1H), 5.07 (s, 1H), 4.40-4.30 (m, 2H), 4.13 (dd, J=6.8, 10.4 Hz, 1H), 4.09-3.99 (m, 3H), 3.08 (br d, J=9.6 Hz, 2H), 3.01 (s, 1H), 2.87-2.77 (m, 1H), 2.39-2.29 (m, 1H), 2.19-2.09 (m, 2H), 2.05 (br d, J=2.0 Hz, 1H), 2.01 (br s, 1H), 1.91-1.74 (m, 5H), 1.74-1.66 (m, 2H), 1.43 (br d, J=12.4 Hz, 2H), 1.23 (s, 3H), 0.72 (t, J=7.6 Hz, 3H); LCMS (ESI, M+1): m/z=634.7.

### Example 847

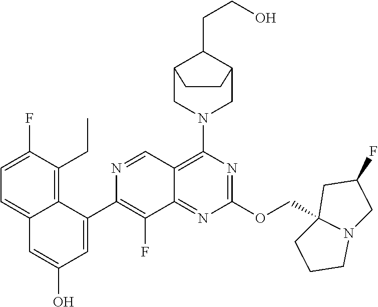 [SMILES: CCc1c(F)ccc2cc(O)cc(-c3ncc4c(N5CC6CCC(C5)C6CCO)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-((1R,5S,8S)-8-(2-hydroxyethyl)-3-azabicyclo[3.2.1]octan-3-yl)pyrido[4,3-d]pyrimidin-7-yl)naphthalen-2-ol

Synthesized according to Example 842. The title compound was obtained as yellow solid (FA salt). ¹H NMR (400 MHZ, METHANOL-d₄) δ=9.15 (s, 1H), 8.53 (br s, 1H), 7.67 (dd, J=6.0, 8.8 Hz, 1H), 7.30 (d, J=2.4 Hz, 1H), 7.25 (t, J=9.2 Hz, 1H), 7.06 (d, J=2.4 Hz, 1H), 5.49-5.25 (m, 1H), 4.50 (br t, J=11.2 Hz, 2H), 4.42-4.37 (m, 1H), 4.35-4.28 (m, 1H), 3.91 (br dd, J=8.8, 11.6 Hz, 2H), 3.76 (t, J=6.4 Hz, 2H), 3.37 (br d, J=6.4 Hz, 3H), 3.16-3.06 (m, 1H), 2.55-2.26 (m, 5H), 2.24-2.13 (m, 2H), 2.07 (q, J=6.4 Hz, 4H), 1.95 (br dd, J=4.0, 6.8 Hz, 2H), 1.89-1.80 (m, 2H), 1.61 (br d, J=10.0 Hz, 2H), 0.79 (dt, J=2.4, 7.2 Hz, 3H); LCMS (ESI, M+1): m/z=648.4.

### Example 848

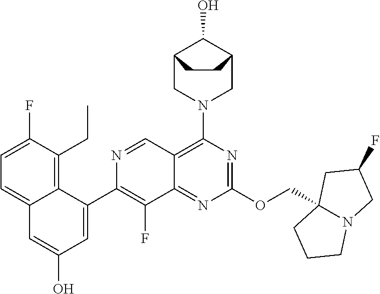 [SMILES: CCc1c(F)ccc2cc(O)cc(-c3ncc4c(N5C[C@H]6CC[C@@H](C5)[C@H]6O)nc(OC[C@@]56CCCN5C[C@H](F)C6)nc4c3F)c12]

### (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-ol

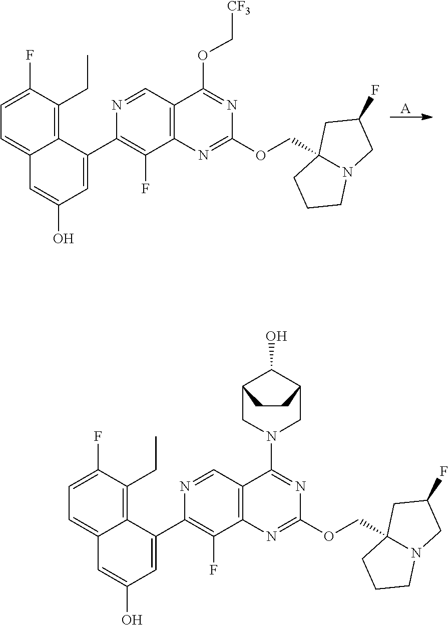

Synthesized according to Example 842. The title compound was obtained as yellow solid (FA salt). ¹H NMR (400 MHZ, DMSO-d6) δ=9.15 (s, 1H), 7.75 (dd, J=6.0, 9.2 Hz, 1H), 7.44-7.28 (m, 2H), 7.02 (d, J=2.0 Hz, 1H), 5.36-5.18 (m, 1H), 4.33 (br s, 2H), 4.13 (dd, J=6.0, 10.0 Hz, 1H), 4.08-3.88 (m, 4H), 3.04-3.04 (m, 1H), 3.14-3.03 (m, 2H), 3.00 (s, 1H), 2.85-2.78 (m, 1H), 2.39-2.29 (m, 1H), 2.20-1.94 (m, 6H), 1.89-1.61 (m, 5H), 1.54-1.34 (m, 2H), 0.72 (t, J=7.2 Hz, 3H); LCMS (ESI, M+1): m/z=620.2

### Example 849

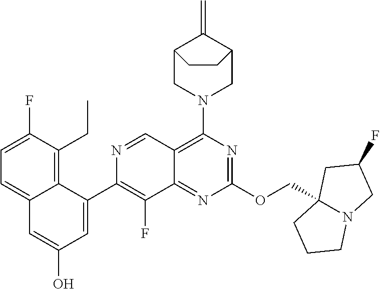 [SMILES: C=C1C2CCC1CN(c1nc(OC[C@@]34CCCN3C[C@H](F)C4)nc3c(F)c(-c4cc(O)cc5ccc(F)c(CC)c45)ncc13)C2]

### 5-ethyl-6-fluoro-4-(8-fluoro-2-(((2R,7aS)-2-fluorotetrahydro-1H-pyrrolizin-7a(5H)-yl)methoxy)-4-((1R,5S)-8-methylene-3-azabicyclo[3.2.1]octan-3-yl)pyrido[4,3-d]pyrimidin-7-yl)naphthalen-2-ol

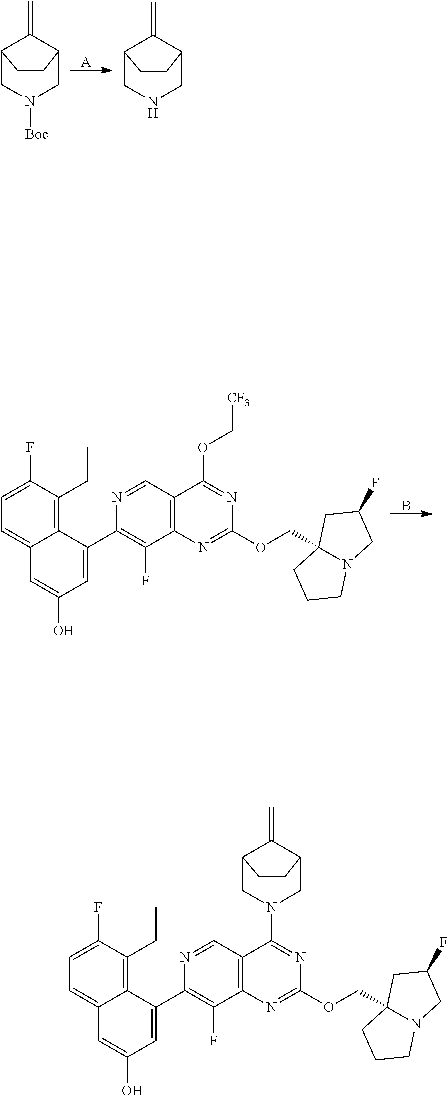

Step A. (1R,5S)-8-methylene-3-azabicyclo[3.2.1]octane: A mixture of (1R,5S)-tert-butyl 8-methylene-3-azabicyclo[3.2.1]octane-3-carboxylate (300 mg, 1.34 mmol, 1.0 equiv) and HCl·dioxane (4 M, 1.0 equiv) in MeCN (0.5 mL) was stirred at 20° C. for 0.5 hour. The mixture was concentrated to afford the title compound (200 mg, crude) as yellow solid.

Step B. 5-ethyl-6-fluoro-4-(8-fluoro-2-(((2R,7aS)-2-fluorohexahydro-1H-pyrrolizin-7a-yl)methoxy)-4-((1R,5S)-8-methylene-3-azabicyclo[3.2.1]octan-3-yl)pyrido[4,3-d]pyrimidin-7-yl)naphthalen-2-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) and (1R,5S)-8-methylene-3-azabicyclo[3.2.1]octane (10.4 mg, 1.0 equiv) in DMF (0.5 mL) were added DIEA (65.4 mg, 6.0 equiv) and 4 Å molecular sieve (10 mg). The reaction was stirred 40° C. for 12 hours. The mixture was filtered and purified by prep-HPLC (column: Phenomenex luna C18 150*25 mm*10 um; mobile phase: [water (FA)-ACN]; B %: 25%-55%, 10 min) to afford the title compound (21.7 mg, 94% yield, 0.41 HCOOH) as yellow solid ¹H NMR (400 MHz, DMSO-d6) δ=9.09 (s, 1H), 8.24 (s, 1H), 7.76 (dd, J=6.0, 9.2 Hz, 1H), 7.44-7.21 (m, 2H), 7.11-6.89 (m, 1H), 5.38-5.16 (m, 1H), 4.87-4.66 (m, 3H), 4.22-4.00 (m, 2H), 3.67-3.59 (m, 2H), 3.14-2.89 (m, 4H), 2.86-2.78 (m, 1H), 2.77-2.68 (m, 2H), 2.41-1.96 (m, 6H), 1.89-1.56 (m, 7H), 0.72 (t, J=7.2 Hz, 3H); LCMS (ESI, M+1): m/z=616.7.

### Example 850

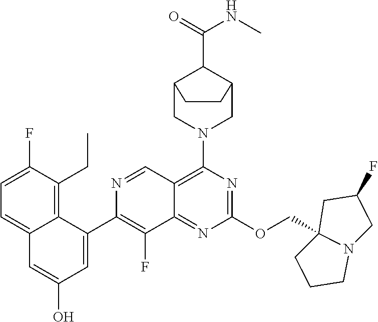 [SMILES: CCc1c(F)ccc2cc(O)cc(-c3ncc4c(N5CC6CCC(C5)C6C(=O)NC)nc(OC[C@@]56CCCN5C[C@H](F)C6)nc4c3F)c12]

### (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.1]octane-8-carboxamide

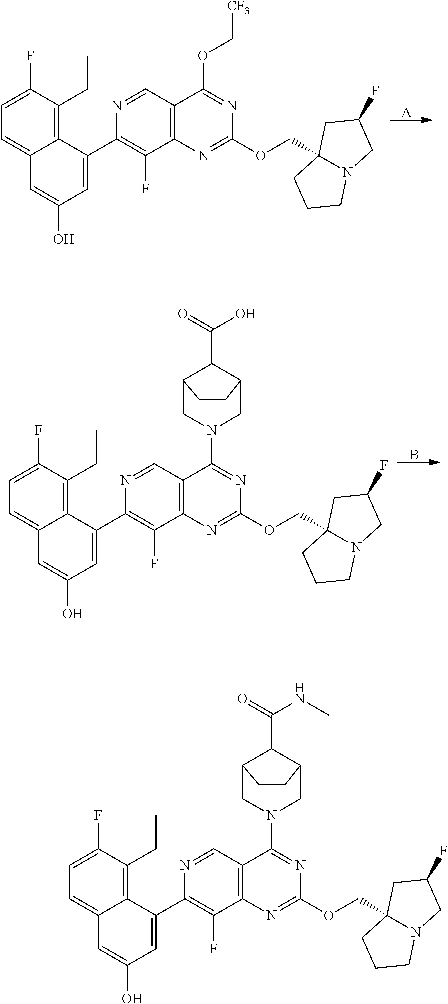

Step A. (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxylic acid: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (300 mg, 1.0 equiv) and (1R,5S,8r)-3-azabicyclo[3.2.1]octane-8-carboxylic acid (194. mg, 2.0 equiv, HCl) in DMF (0.1 mL) were added DIEA (654 mg, 10 equiv) and 4 Å molecular sieve (10.0 mg). The reaction was stirred at 60° C. for 12 hours. The mixture was filtered and purified by prep-HPLC (column: Welch Xtimate C18 150× 25 mm×5 um; mobile phase: [water (NH₃H₂O)-ACN]; B %: 10%-40%, 8 min) to afford the title compound (115 mg, 33% yield) as yellow solid; LCMS (ESI, M+1): m/z=648.4.

Step B. (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N-methyl-3-azabicyclo[3.2.1]octane-8-carboxamide: To a solution of (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxylic acid (50.0 mg, 1.0 equiv) and methanamine (10.4 mg, 2.0 equiv, HCl) in DMF (0.5 mL) were added HATU (88.1 mg, 3.0 equiv) and DIEA (59.9 mg, 6.0 equiv). The reaction was stirred at 25° C. for 0.5 hour. The mixture was filtered and purified by prep-HPLC (column: Phenomenex luna C18 150×25 mm×10 μm; mobile phase: [water (FA)-ACN]; B %: 20%-50%, 10 min) to afford the title compound (15.9 mg, 30% yield, 0.5 HCOOH) as off-white solid; ¹H NMR (400 MHZ, METHANOL-d4) δ=9.09 (s, 1H), 7.68 (dd, J=6.0, 9.2 Hz, 1H), 7.35-7.20 (m, 2H), 7.05 (d, J=2.4 Hz, 1H), 5.59-5.34 (m, 1H), 4.78 (br d, J=3.6 Hz, 2H), 4.55-4.41 (m, 2H), 3.76-3.47 (m, 5H), 3.27-3.20 (m, 1H), 2.82 (s, 1H), 2.76-2.66 (m, 5H), 2.59-2.36 (m, 3H), 2.34-2.25 (m, 1H), 2.24-2.13 (m, 3H), 2.11-1.96 (m, 1H), 1.92-1.82 (m, 2H), 1.55 (br d, J=8.8 Hz, 2H), 0.79 (dt, J=2.8, 7.2 Hz, 3H); LCMS (ESI, M+1): m/z=661.4.

### Example 851

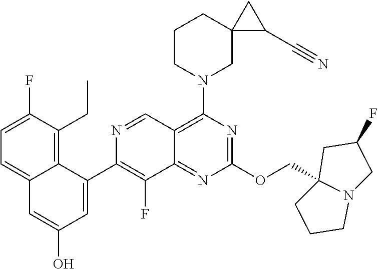 [SMILES: CCc1c(F)ccc2cc(O)cc(-c3ncc4c(N5CCCC6(CC6C#N)C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-5-azaspiro[2.5]octane-1-carbonitrile

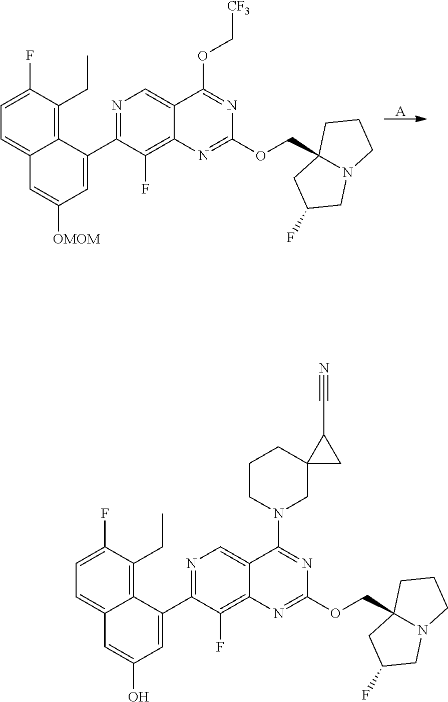

Synthesized according to Example 842. The title compound was obtained as yellow solid. ¹HNMR (400 MHZ, METHANOL-d₄) δ=0.81 (br t, J=6.8 Hz, 3H), 1.13-1.21 (m, 1H), 1.32-1.46 (m, 1H), 1.81-1.89 (m, 1H), 1.90-2.00 (m, 1H), 2.02-2.15 (m, 4H), 2.16-2.31 (m, 3H), 2.33-2.41 (m, 1H), 2.42-2.70 (m, 3H), 3.36 (br s, 1H), 3.64-4.14 (m, 6H), 4.27-4.44 (m, 1H), 4.48-4.68 (m, 2H), 5.37-5.65 (m, 1H), 7.08 (br s, 1H), 7.28 (t, J=9.2 Hz, 1H), 7.33 (d, J=2.8 Hz, 1H), 7.70 (dd, J=9.2, 5.6 Hz, 1H), 8.42 (br s, 1H), 9.11 (d, J=3.6 Hz, 1H); LCMS (ESI, M+1): m/z=629.4.

### Example 852

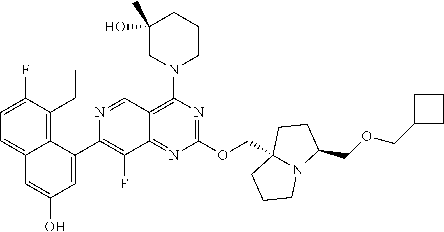 [SMILES: CCc1c(F)ccc2cc(O)cc(-c3ncc4c(N5CCC[C@@](C)(O)C5)nc(OC[C@@]56CCCN5[C@H](COCC5CCC5)CC6)nc4c3F)c12]

### (R)-1-(2-(((3S,7aS)-3-((cyclobutylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

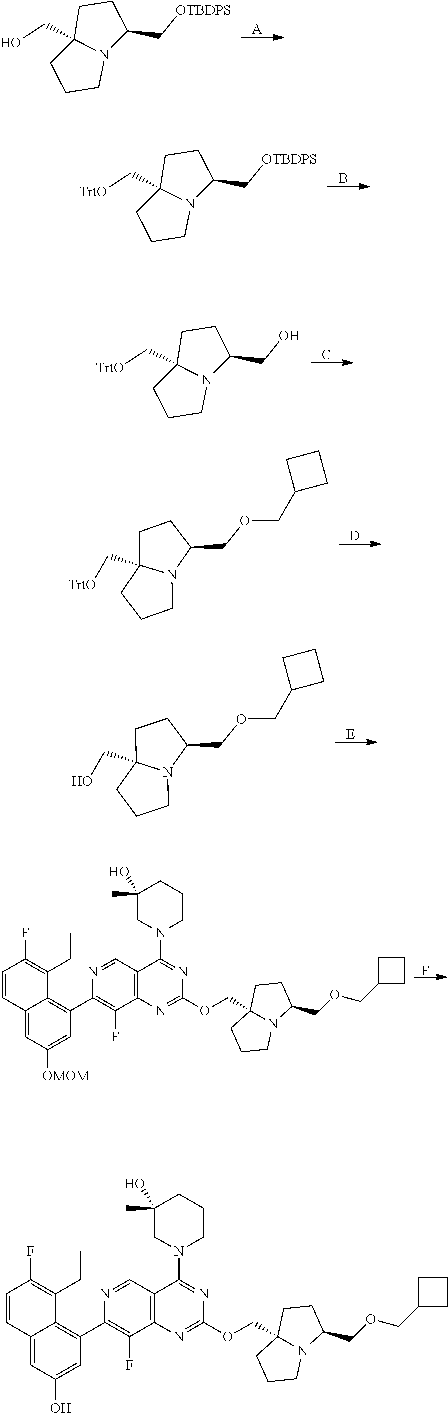

Step A. (3S,7aS)-3-(((tert-butyldiphenylsilyl)oxy)methyl)-7a-((trityloxy)methyl) hexahydro-1H-pyrrolizine: To a solution of ((3S,7aS)-3-(((tert-butyldiphenylsilyl)oxy)methyl)tetrahydro-1H-pyrrolizin-7a(5H)-yl)methanol (16.0 g, 1 equiv) and TEA (7.90 g, 2.0 equiv) in DCM (160 mL) was added [chloro(diphenyl)methyl]benzene (16.3 g, 1.5 equiv). The reaction was stirred at 25° C. for 12 hours. The mixture was diluted with water (20 mL) and extracted with DCM (3×30 mL). The combined organic layers were washed with brine (30 mL), dried over Na₂SO₄, filtered and concentrated under reduced pressure to afford the title compound (25.0 g, 98% yield) as yellow oil liquid.

Step B. ((3S,7aS)-7a-((trityloxy)methyl) hexahydro-1H-pyrrolizin-3-yl)methanol: To a solution of (3S,7aS)-3-(((tert-butyldiphenylsilyl)oxy)methyl)-7a-((trityloxy)methyl) hexahydro-1H-pyrrolizine (24.0 g, 1.0 equiv) in DMF (300 mL) was added CsF (83.9 g, 15 equiv) in one portion under N₂. The reaction was stirred at 40° C. for 6 hours. The mixture was concentrated to remove DMF and purified by reversed phase flash chromatography [C18, 0.1% formic acid condition] to afford the title compound (3.30 g, 20% yield) as white solid; ¹H NMR (400 MHZ, METHANOL-d₄) δ=7.61-7.17 (m, 15H), 3.94-3.72 (m, 3H), 3.59-3.43 (m, 3H), 3.41-3.32 (m, 1H), 3.31 (s, 1H), 2.14-1.79 (m, 8H); LCMS (ESI, M+1): m/z=414.5.

Step C. (3S,7aS)-3-((cyclobutylmethoxy)methyl)-7a-((trityloxy)methyl) hexahydro-1H-pyrrolizine: To a solution of ((3S,7aS)-7a-((trityloxy)methyl) hexahydro-1H-pyrrolizin-3-yl)methanol (470 mg, 1.0 equiv) in THF (2.5 mL) and DMF (2.5 mL) was added NaH (282 mg, 60% purity, 6.2 equiv) at 0° C. under N₂. The reaction was stirred at 0° C. for 1 hour. Then (bromomethyl)cyclobutane (508 mg, 3 equiv) was added. The reaction was stirred at 0-25° C. for 47 hours. The mixture was quenched by water (5.0 mL) at 0° C. and extracted with ethyl acetate (3×3.0 mL). The combined organic layers were washed with brine, dried over anhydrous sodium sulfate, filtered, concentrated and purified by reversed phase flash chromatography [C18, 0.1% formic acid condition] to afford the title compound (85 mg, 16% yield) as yellow oil. ¹H NMR (400 MHZ, CDCl₃-d) δ=7.46 (br d, J=7.6 Hz, 6H), 7.37-7.28 (m, 6H), 7.27-7.20 (m, 3H), 3.88-3.73 (m, 2H), 3.71-3.55 (m, 2H), 3.54-3.43 (m, 2H), 3.22-3.07 (m, 2H), 3.05-2.86 (m, 1H), 2.61-2.48 (m, 1H), 2.13-1.53 (m, 14H).

Step D. ((3S,7aS)-3-((cyclobutylmethoxy)methyl)tetrahydro-1H-pyrrolizin-7a(5H)-yl)methanol: To a solution of (3S,7aS)-3-((cyclobutylmethoxy)methyl)-7a-((trityloxy)methyl) hexahydro-1H-pyrrolizine (110 mg, 1.0 equiv) in DCM (1.1 mL) was added TFA (260 mg, 10 equiv). The reaction was stirred at 0-25° C. for 12 hours. The mixture was diluted with water (2.0 mL) and extracted with ethyl acetate (3×2.0 mL). The combined organic layers were washed with brine, dried over anhydrous sodium sulfate, filtered, concentrated and purified by column chromatography (Al₂O₃, petroleum ether/ethyl acetate=10/1 to 1/1, dichloromethane/methanol=15:1) to afford the title compound (55 mg, crude) as yellow oil.

Step E. (R)-1-(2-(((3S,7aS)-3-((cyclobutylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (80.0 mg, 1.0 equiv) and ((3S,7aS)-3-((cyclobutylmethoxy)methyl)tetrahydro-1H-pyrrolizin-7a(5H)-yl)methanol (43.4 mg, 1.2 equiv) in toluene (0.80 mL) was added t-BuONa (2.0 M, 151 μL, 2.0 equiv) at 0° C. The reaction was stirred at 0° C. for 1 hour. The mixture was diluted with water (2.0 mL) and extracted with ethyl acetate (3×2.0 mL). The combined organic layers were washed with brine, dried over anhydrous sodium sulfate, filtered and concentrated to afford the title compound (95 mg, crude) as yellow oil; LCMS (ESI, M+1): m/z=732.3.

Step F. (R)-1-(2-(((3S,7aS)-3-((cyclobutylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a solution of (R)-1-(2-(((3S,7aS)-3-((cyclobutylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (95.0 mg, 1.0 equiv) in MeCN (0.95 mL) was added HCl·dioxane (4.0 M, 1.9 mL, 59 equiv) at 0° C. The reaction was stirred at 0° C. for 1 hour. The mixture was quenched with iced saturated NaHCO₃ solution (5.0 mL) dropwise at 0° C. and extracted with ethyl acetate (3×3.0 mL). The combined organic layers were washed with brine, dried over anhydrous sodium sulfate, filtered, concentrated under vacuum and purified by reversed-phase HPLC [column: Phenomenex luna C18 150×25 mm×10 um; mobile phase: water (FA)-ACN; B %: 24%-54%, 10 min] to afford the title compound (12.5 mg, 13% yield, HCOOH) as an off-white solid; ¹H NMR (400 MHZ, METHANOL-d₄) δ=9.26 (d, J=1.2 Hz, 1H), 7.70 (dd, J=6.0, 9.2 Hz, 1H), 7.32 (d, J=2.4 Hz, 1H), 7.27 (t, J=9.6 Hz, 1H), 7.07 (d, J=2.4 Hz, 1H), 5.06 (br s, 1H), 4.71-4.62 (m, 1H), 4.57-4.49 (m, 1H), 4.35 (br dd, J=9.2, 12.8 Hz, 1H), 4.07-3.95 (m, 1H), 3.76-3.67 (m, 2H), 3.66-3.59 (m, 1H), 3.53-3.48 (m, 2H), 3.46 (br s, 1H), 3.36 (br s, 2H), 2.67-2.54 (m, 1H), 2.52-2.42 (m, 1H), 2.37-2.23 (m, 3H), 2.20-2.10 (m, 3H), 2.08-1.93 (m, 7H), 1.89-1.71 (m, 6H), 1.31 (d, J=8.8 Hz, 3H), 0.88-0.75 (m, 3H); LCMS (ESI, M+1): m/z=688.7.

### Example 853

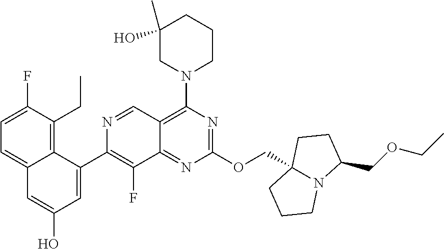 [SMILES: CCOC[C@@H]1CC[C@]2(COc3nc(N4CCC[C@@](C)(O)C4)c4cnc(-c5cc(O)cc6ccc(F)c(CC)c56)c(F)c4n3)CCCN12]

### (R)-1-(2-(((3S,7aS)-3-(ethoxymethyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

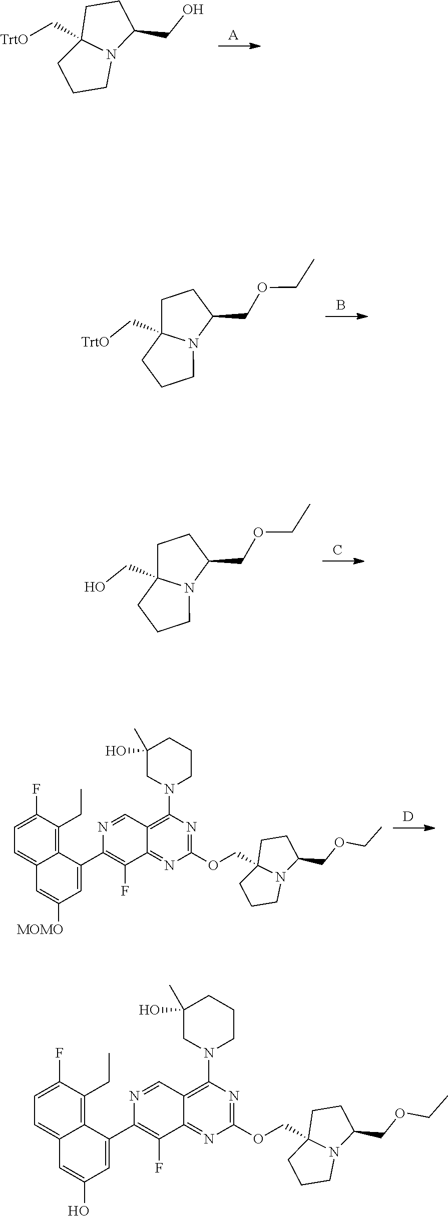

Step A. (3S,7aS)-3-(ethoxymethyl)-7a-((trityloxy)methyl) hexahydro-1H-pyrrolizine: To a suspension of NaH (58.03 mg, 1.45 mmol, 60% purity, 1.2 equiv) in THF (5 mL) and DMF (5 mL) was added ((3S,7aS)-7a-((trityloxy)methyl) hexahydro-1H-pyrrolizin-3-yl)methanol (500 mg, 1.0 equiv) in one portion at 0° C. under N₂. The reaction was stirred at 0° C. for 30 minutes. To above mixture was added iodoethane (283 mg, 1.5 equiv). The reaction was warmed to 20° C. and stirred for 12 hours. The mixture was diluted with ice water (4 mL), adjusted to pH=1 with con. HCl, extracted with ethyl acetate (15 mL×3), washed with brine (15 mL), dried over Na₂SO₄, filtered and purified by reversed phase flash chromatography [C18, 0.1% formic acid condition] to afford the title compound (200 mg, 36% yield) as white solid; LCMS (ESI, M+1): m/z=442.3.

Step B. ((3S,7aS)-3-(ethoxymethyl)tetrahydro-1H-pyrrolizin-7a(5H)-yl)methanol: To a mixture of (3S,7aS)-3-(ethoxymethyl)-7a-((trityloxy)methyl) hexahydro-1H-pyrrolizine (200 mg, 1.0 equiv) in DCM (2 mL) was added TFA (516 mg, 10 equiv) in one portion under N₂. The reaction was stirred at 20° C. for 12 hours. The mixture was concentrated, dissolved in MeOH (5 mL), neutralized with solid NaHCO₃, filtered and purified by column chromatography (Al₂O₃, Petroleum ether/Ethyl acetate=1/0 to 0:1) to afford the title compound (90 mg, 99% yield) as yellow solid; ¹H NMR (400 MHZ, DMSO-d₆) δ=3.60-3.54 (m, 1H), 3.52-3.40 (m, 4H), 3.29-3.19 (m, 3H), 2.96-2.74 (m, 2H), 1.98-1.91 (m, 1H), 1.83-1.54 (m, 6H), 1.53-1.40 (m, 1H), 1.15-1.07 (m, 3H).

Step C. (R)-1-(2-(((3S,7aS)-3-(ethoxymethyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of ((3S,7aS)-3-(ethoxymethyl)tetrahydro-1H-pyrrolizin-7a(5H)-yl)methanol and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (239 mg, 1.0 equiv) in toluene (1 mL) was added t-BuONa (2 M, 2.0 equiv) in one portion at 0° C. under N₂. The reaction was stirred at 0° C. for 1 hour. The mixture was diluted with water (20 mL) and extracted with ethyl acetate (3×30 mL). The combined organic layers were washed with brine (30 mL), dried over Na₂SO₄, filtered, concentrated under reduced pressure and purified by prep-HPLC [column: Phenomenex luna C18 150×25 mm×10 um; mobile phase: water (FA)-ACN; B %: 25%-45%, 58 min] to afford the title compound (90 mg. 27% yield) as yellow solid; LCMS (ESI, M+1): m/z=692.8.

Step D. (R)-1-(2-(((3S,7aS)-3-(ethoxymethyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of (R)-1-(2-(((3S,7aS)-3-(ethoxymethyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (80.0 mg, 1.0 equiv) in ACN (0.8 mL) was added HCl/dioxane (4 M, 6.9 equiv) in one portion at 0° C. under N₂. The reaction was stirred at 20° C. for 1 hours. The mixture was diluted with water (20 mL) and extracted with ethyl acetate (3×30 mL). The combined organic layers were washed with brine (30 mL), dried over Na₂SO₄, filtered, concentrated under reduced pressure and purified by prep-HPLC [column: Phenomenex luna C18 150×25 mm×10 um; mobile phase: water (FA)-ACN; B %: 20%-50%, 10 min] to afford the title compound (15.9 mg, 21% yield, HCOOH) as pink solid; ¹H NMR (400 MHZ, METHANOL-d₄) δ=9.25 (s, 1H), 8.55 (s, 1H), 7.71-7.65 (m, 1H), 7.31 (d, J=2.8 Hz, 1H), 7.29-7.22 (m, 1H), 7.06 (s, 1H), 4.65-4.60 (m, 1H), 4.55 (br s, 1H), 4.50-4.44 (m, 1H), 4.34 (br t, J=11.6 Hz, 1H), 3.99-3.85 (m, 1H), 3.75-3.67 (m, 2H), 3.63 (br d, J=11.2 Hz, 1H), 3.59-3.51 (m, 2H), 3.49-3.41 (m, 1H), 3.22-3.16 (m, 1H), 2.53-2.41 (m, 1H), 2.36-2.28 (m, 1H), 2.27-2.13 (m, 3H), 2.12-2.00 (m, 3H), 1.99-1.85 (m, 4H), 1.84-1.75 (m, 2H), 1.30 (d, J=9.2 Hz, 3H), 1.24-1.14 (m, 3H), 0.86-0.75 (m, 3H); LCMS (ESI, M+1): m/z=648.7.

### Example 854

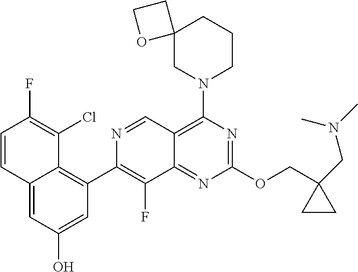 [SMILES: CN(C)CC1(COc2nc(N3CCCC4(CCO4)C3)c3cnc(-c4cc(O)cc5ccc(F)c(Cl)c45)c(F)c3n2)CC1]

### 5-chloro-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-6-fluoronaphthalen-2-ol

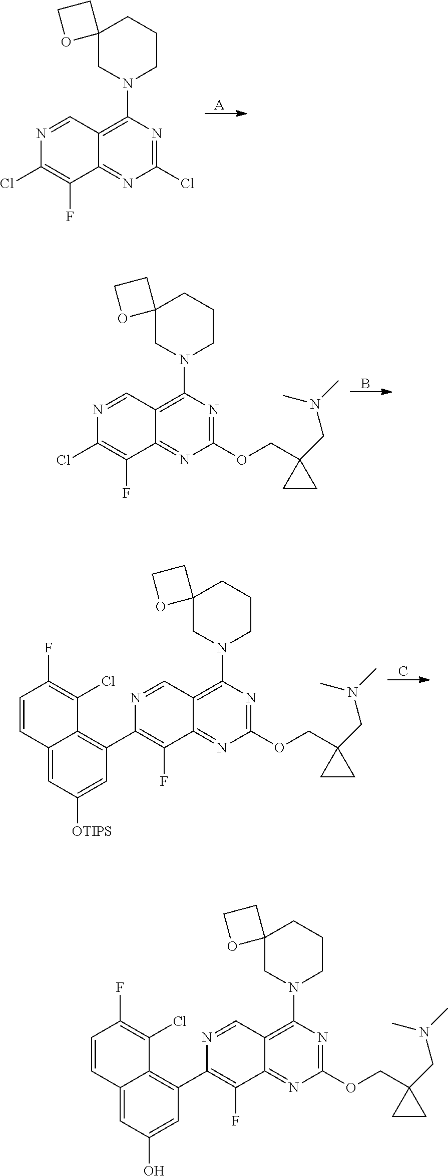

Step A. 1-(1-(((7-chloro-8-fluoro-4-(1-oxa-6-azaspiro[3.5]nonan-6-yl)pyrido[4,3-d]pyrimidin-2-yl)oxy)methyl)cyclopropyl)-N,N-dimethylmethanamine: To a mixture of 6-(2,7-dichloro-8-fluoropyrido[4,3-d]pyrimidin-4-yl)-1-oxa-6-azaspiro[3.5]nonane (2.00 g, 1.0 equiv) and [1-[(dimethylamino)methyl]cyclopropyl]methanol (753 mg, 1.0 equiv) in dioxane (20 mL) was added DIEA (2.26 g, 3.0 equiv). The reaction was stirred at 90° C. for 2 hours. The mixture was concentrated and purified by reversed phase flash chromatography [C18, 0.1% formic acid condition] to afford the title compound (436 mg, 12% yield, FA salt) as yellow oil; ¹H NMR (400 MHz, DMSO-d6) δ=9.01 (s, 1H), 4.42-4.29 (m, 4H), 4.26 (s, 2H), 4.18-4.12 (m, 1H), 3.81 (br d, J=13.6 Hz, 1H), 2.35-2.29 (m, 4H), 2.22 (s, 6H), 2.13-2.02 (m, 2H), 1.88-1.77 (m, 2H), 0.69-0.63 (m, 2H), 0.47-0.42 (m, 2H); LCMS (ESI, M+1): m/z=436.2.

Step B. 1-(1-(((7-(8-chloro-7-fluoro-3-((triisopropylsilyl)oxy)naphthalen-1-yl)-8-fluoro-4-(1-oxa-6-azaspiro[3.5]nonan-6-yl)pyrido[4,3-d]pyrimidin-2-yl)oxy)methyl)cyclopropyl)-N,N-dimethylmethanamine: To a mixture of 1-(1-(((7-chloro-8-fluoro-4-(1-oxa-6-azaspiro[3.5]nonan-6-yl)pyrido[4,3-d]pyrimidin-2-yl)oxy)methyl)cyclopropyl)-N,N-dimethylmethanamine (200 mg, 1.0 equiv), CuI (26.2 mg, 0.3 equiv) and ((5-chloro-6-fluoro-4-(trimethylstannyl)naphthalen-2-yl)oxy)triisopropylsilane (592 mg, 2.5 equiv) in toluene (3 mL) were added BINAP (57.1 mg, 0.2 equiv) and [1,1-Bis(diphenylphosphino)ferrocene]dichloropalladium(II) (33.6 mg, 0.1 equiv). The reaction was stirred at 90° C. for 16 hours under N₂. The mixture was diluted with water (10 mL) and extracted with ethyl acetate (2×20 mL). The combined organic layers were dried over anhydrous sodium sulfate, filtered, concentrated and purified by reversed phase flash chromatography [C18, 0.1% formic acid condition] to afford the title compound (151 mg, 28.4% yield) as yellow solid; ¹H NMR (400 MHZ, CHLOROFORM-d) δ=9.28-9.17 (m, 1H), 7.73 (br d, J=6.0 Hz, 2H), 7.40 (br s, 2H), 4.63-4.54 (m, 2H), 4.46-4.35 (m, 3H), 4.31-4.18 (m, 3H), 3.63-3.45 (m, 2H), 2.66-2.59 (m, 2H), 2.51-2.46 (m, 2H), 2.10-2.04 (m, 2H), 1.54 (s, 6H), 1.35-1.30 (m, 3H), 1.13 (d, J=7.2 Hz, 18H), 0.98-0.81 (m, 4H); LCMS (ESI, M+1): m/z=752.4.

Step C. 5-chloro-4-(2-((1-((dimethylamino)methyl)cyclopropyl)methoxy)-8-fluoro-4-(1-oxa-6-azaspiro[3.5]nonan-6-yl)pyrido[4,3-d]pyrimidin-7-yl)-6-fluoronaphthalen-2-ol: To a solution of 1-(1-(((7-(8-chloro-7-fluoro-3-((triisopropylsilyl)oxy)naphthalen-1-yl)-8-fluoro-4-(1-oxa-6-azaspiro[3.5]nonan-6-yl)pyrido[4,3-d]pyrimidin-2-yl)oxy)methyl)cyclopropyl)-N,N-dimethylmethanamine (130 mg, 1.0 equiv) in DMF (1 mL) was added CsF (105 mg, 4.0 equiv). The reaction was stirred at 25° C. for 1 hour. The mixture was filtered and purified by prep-HPLC [column: Phenomenex luna C18 150×25 mm×10 um; mobile phase: water (FA)-ACN; B %: 16%-46%, 10 min] to afford the title compound (6.35 mg. 5.9% yield) as yellow solid; ¹H NMR (400 MHZ, METHANOL-d4) δ=9.27 (d, J=10.4 Hz, 1H), 7.82 (dd, J=5.6, 9.2 Hz, 1H), 7.47-7.39 (m, 1H), 7.38 (d, J=2.4 Hz, 1H), 7.24 (d, J=2.4 Hz, 1H), 4.73-4.64 (m, 1H), 4.63-4.58 (m, 2H), 4.57-4.49 (m, 2H), 4.46-4.37 (m, 2H), 3.82 (dd, J=5.2, 13.6 Hz, 1H), 3.51-3.42 (m, 1H), 2.94-2.75 (m, 2H), 2.71-2.53 (m, 6H), 2.52-2.45 (m, 1H), 2.36-2.24 (m, 1H), 2.09-1.91 (m, 2H), 1.89-1.76 (m, 1H), 0.90-0.82 (m, 2H), 0.69 (br s, 2H); LCMS (ESI, M+1): m/z=596.5.

### Example 855

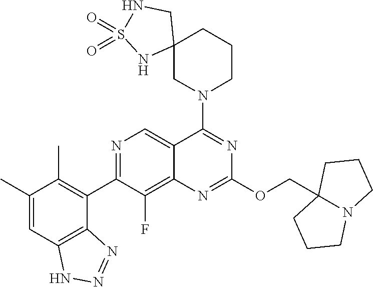 [SMILES: Cc1cc2[nH]nnc2c(-c2ncc3c(N4CCCC5(CNS(=O)(=O)N5)C4)nc(OCC45CCCN4CCC5)nc3c2F)c1C]

### 7-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-2-thia-1,3,7-triazaspiro[4.5]decane 2,2-dioxide

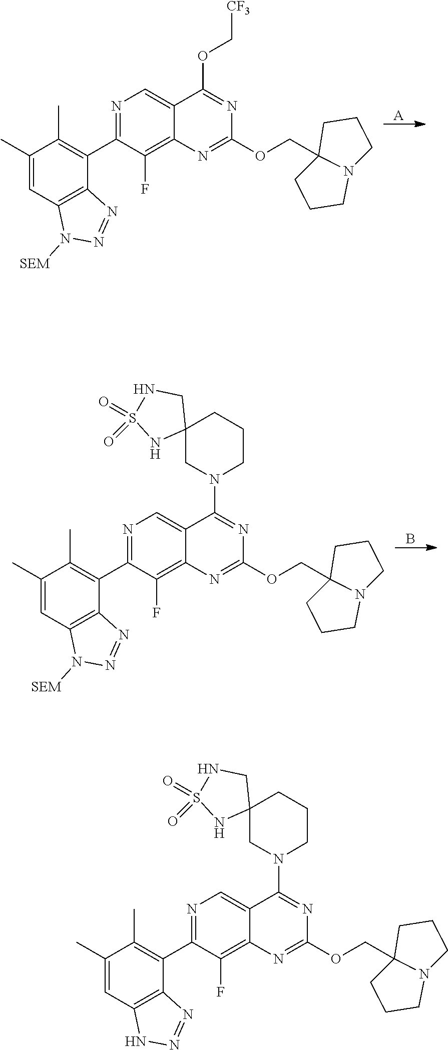

Step A. 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide: To a solution of 7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)-4-(2,2,2-trifluoroethoxy)pyrido[4,3-d]pyrimidine (300 mg, 1.0 equiv) and 2-thia-1,3,7-triazaspiro[4.5]decane 2,2-dioxide (130 mg, 1.5 equiv) in DMF (0.5 mL) was added DIEA (176 mg, 3.0 equiv). The reaction was stirred at 40° C. for 12 hours. The mixture was filtered and purified with reversed phase flash chromatography [C18, 0.1% formic acid condition] to afford the title compound (300 mg, 83% yield) as yellow solid; LCMS (ESI, M+1): m/z=753.4.

Step B. 7-(7-(5,6-dimethyl-1H-benzo[d][1,2,3]triazol-4-yl)-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide: To a solution of 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-2-thia-1,3,7-triazaspiro[4.5]decane 2,2-dioxide (100 mg, 1.0 equiv) in THF (1 mL) was added TBAF (1M, 0.7 mL, 5.0 equiv). The reaction was stirred at 50° C. for 12 hours. The mixture was concentrated and purified with prep-HPLC [column: Waters Xbridge 150×25 mm×5 um; A: water (NH₄HCO₃), B: ACN, B %: 22%-52% over 8 min] to afford the title compound (5.01 mg, 5.6% yield) as white solid; ¹H NMR (400 MHZ, METHANOL-d₄) δ=9.19 (s, 1H), 7.79 (s, 1H), 4.54-4.31 (m, 4H), 3.48 (s, 2H), 3.30-3.13 (m, 4H), 2.93-2.80 (m, 2H), 2.55-2.49 (m, 3H), 2.29-2.22 (m, 3H), 2.20-2.10 (m, 2H), 2.08-1.90 (m, 7H), 1.89-1.79 (m, 3H); LCMS (ESI, M+1): m/z=623.5.

### Example 856

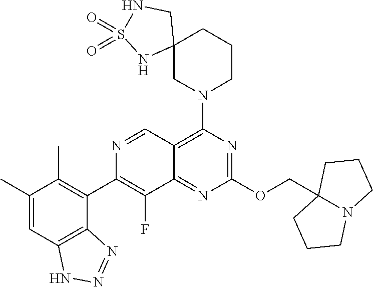 [SMILES: Cc1cc2[nH]nnc2c(-c2ncc3c(N4CCCC5(CNS(=O)(=O)N5)C4)nc(OCC45CCCN4CCC5)nc3c2F)c1C]

### 7-(7-(5,6-dimethyl-1H-benzo[d][1,2,3]triazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decan-2-one

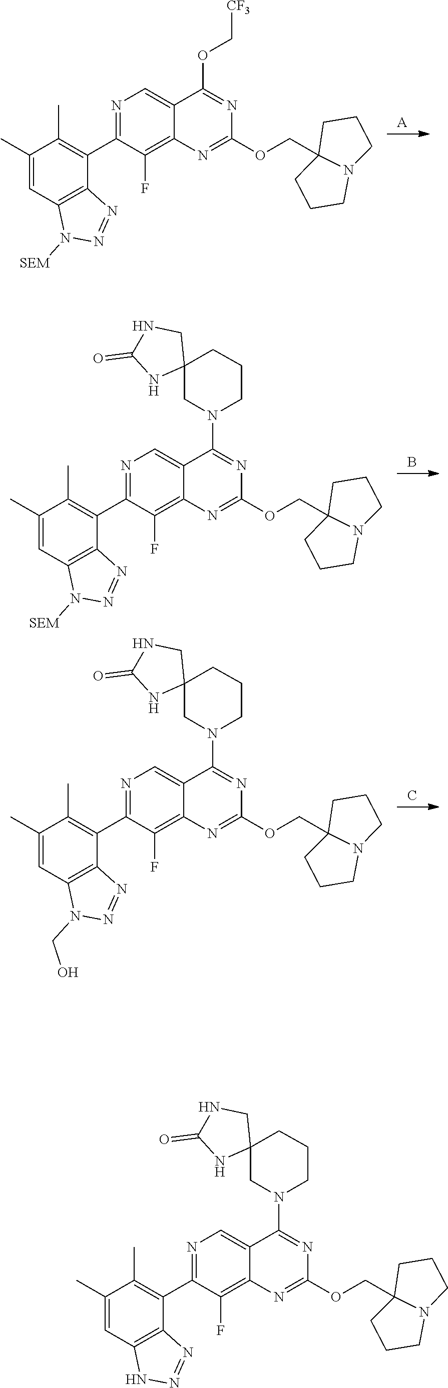

Step A. 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-1,3,7-triazaspiro[4.5]decan-2-one: To a mixture of 7-(5,6-dimethyl-1-((2-(trimethylsilyl) ethoxy)methyl)-1H-benzo[d][1,2,3]triazol-4-yl)-8-fluoro-2-((tetrahydro-1H-pyrrolizin-7a(5H)-yl)methoxy)-4-(2,2,2-trifluoroethoxy)pyrido[4,3-d]pyrimidine (300 mg, 1.0 equiv) and 1,3,9-triazaspiro[4.5]decan-2-one (141 mg, 2.0 equiv) in DMF (1 mL) were added 4 Å molecular sieve (50 mg) and DIEA (176 mg, 3.0 equiv). The reaction was stirred at 50° C. for 12 hours. The mixture was filtered and purified with reversed phase flash chromatography [C18, 0.1% formic acid condition] to afford the title compound (150 mg, 45% yield) as yellow solid; LCMS (ESI, M+1): m/z=717.5.

Step B. 7-(8-fluoro-2-((hexahydro-1H-pyrrolizin-7a-yl)methoxy)-7-(1-(hydroxymethyl)-5,6-dimethyl-1H-benzo[d][1,2,3]triazol-4-yl)pyrido[4,3-d]pyrimidin-4-yl)-1,3,7-triazaspiro[4.5]decan-2-one: To a mixture of 7-(7-(5,6-dimethyl-1-((2-(trimethylsilyl) ethoxy)methyl)-1H-benzo[d][1,2,3]triazol-4-yl)-8-fluoro-2-((hexahydro-1H-pyrrolizin-7a-yl)methoxy)pyrido[4,3-d]pyrimidin-4-yl)-1,3,7-triazaspiro[4.5]decan-2-one (60.0 mg, 1.0 equiv) in THF (0.5 mL) was added TBAF (1 M, 418 μL, 5.0 equiv) at 0° C. The reaction was stirred at 50° C. for 12 hours. The mixture was diluted with water (1 mL) and extracted with dichloromethane/methanol=10/1 (3×2 mL). The combined organic layers were dried over anhydrous sodium sulfate, concentrated and purified with reversed phase flash chromatography [C18, 0.1% formic acid condition] to afford the title compound (60.0 mg, 96% yield); LCMS (ESI, M+1): m/z=617.2.

Step C. 7-(7-(5,6-dimethyl-1H-benzo[d][1,2,3]triazol-4-yl)-8-fluoro-2-((hexahydro-1H-pyrrolizin-7a-yl)methoxy)pyrido[4,3-d]pyrimidin-4-yl)-1,3,7-triazaspiro[4.5]decan-2-one: To a solution of 7-(8-fluoro-2-((hexahydro-1H-pyrrolizin-7a-yl)methoxy)-7-(1-(hydroxymethyl)-5,6-dimethyl-1H-benzo[d][1,2,3]triazol-4-yl)pyrido[4,3-d]pyrimidin-4-yl)-1,3,7-triazaspiro[4.5]decan-2-one (60.0 mg, 1.0 equiv) in THF (0.2 mL) was added NH₃·H₂O (455 mg, 25% purity, 33 equiv). The reaction was stirred at 20° C. for 24 hours. The mixture was diluted with water (2 mL) and extracted with dichloromethane (3×3 mL). The combined organic layers were dried over anhydrous sodium sulfate, concentrated and purified with prep-HPLC [Waters Xbridge 150×25 mm×5 μm; A: water (NH₄HCO₃), B: ACN, B %: 19%-49% over 8 min] and prep-HPLC [Phenomenex Luna C18 150×25 mm×10 μm; A: water (FA), B: ACN, B %: 5%-35% over 9 min] to afford the title compound (7.73 mg, 13% yield, 0.72 HCOOH) as green solid; ¹H NMR (400 MHZ, METHANOL-d₄) δ=9.22 (s, 1H), 7.80 (s, 1H), 4.65-4.48 (m, 3H), 4.36-4.06 (m, 2H), 4.03-3.81 (m, 2H), 3.60-3.50 (m, 2H), 3.44 (br d, J=9.6 Hz, 1H), 3.14 (td, J=5.6, 11.2 Hz, 2H), 2.54 (s, 3H), 2.28 (s, 3H), 2.27-2.18 (m, 2H), 2.18-2.05 (m, 4H), 2.05-1.80 (m, 6H); LCMS (ESI, M+1): m/z=587.4.

### Example 857

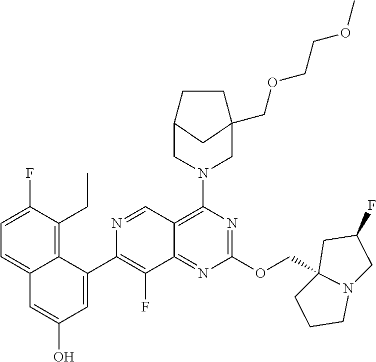 [SMILES: CCc1c(F)ccc2cc(O)cc(-c3ncc4c(N5CC6CCC(COCCOC)(C6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-((2-methoxyethoxy)methyl)-3-azabicyclo[3.2.1]octan-3-yl)pyrido[4,3-d]pyrimidin-7-yl)naphthalen-2-ol

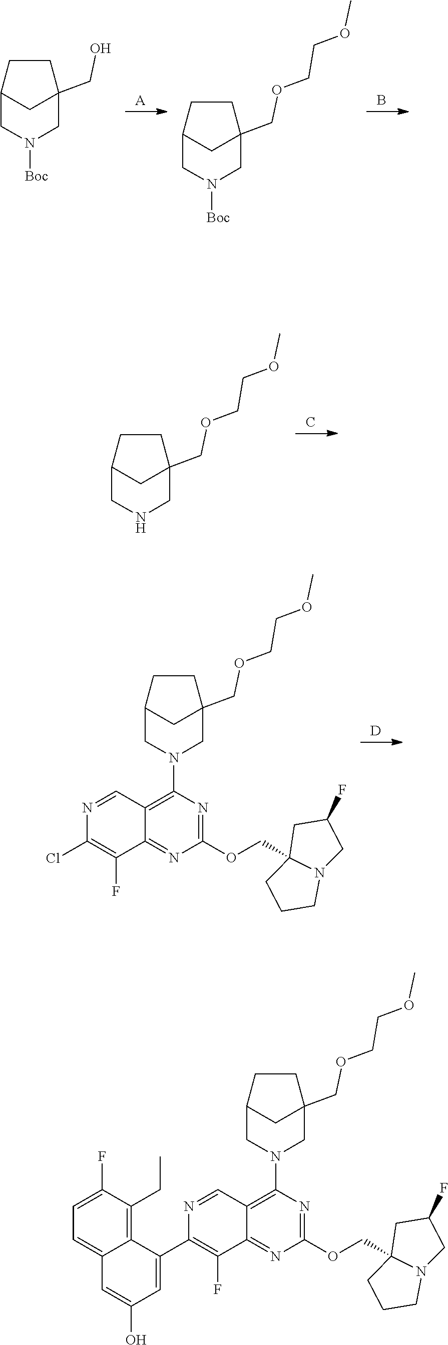

Step A. tert-butyl 1-((2-methoxyethoxy)methyl)-3-azabicyclo[3.2.1]octane-3-carboxylate: To a solution of tert-butyl 1-(hydroxymethyl)-3-azabicyclo[3.2.1]octane-3-carboxylate (500 mg, 1.0 equiv) in DMAC (10 mL) was added NaH (249 mg, 60% purity, 3.0 equiv) portionwise at 0° C. The reaction was stirred at 0° C. for 0.5 hours. 1-bromo-2-methoxyethane (576 mg, 2.0 equiv) was added at 0° C. The reaction was stirred at 25° C. for 3 hours. The mixture was quenched with water (25 mL) and extracted with ethyl acetate (50 mL). The organic layer was dried over sodium sulfate, concentrated and purified with column chromatography (SiO₂, Petroleum ether/Ethyl acetate=15/1 to 3/1) to afford the title compound (470 mg, 76% yield) as colourless oil.

Step B. 1-((2-methoxyethoxy)methyl)-3-azabicyclo[3.2.1]octane: To a solution of tert-butyl 1-((2-methoxyethoxy)methyl)-3-azabicyclo[3.2.1]octane-3-carboxylate (200 mg, 1.0 equiv) in MeCN (3 mL) was added HCl/dioxane (4 M, 7 mL, 42 equiv) at 0° C. The reaction was stirred at 0° C. for 0.5 hours. The mixture was concentrated to afford the title compound (157 mg, crude, HCl) as white solid.

Step C. 7-chloro-8-fluoro-2-(((2R,7aS)-2-fluorotetrahydro-1H-pyrrolizin-7a(5H)-yl)methoxy)-4-(1-((2-methoxyethoxy)methyl)-3-azabicyclo[3.2.1]octan-3-yl)pyrido[4,3-d]pyrimidine: To a solution of 1-((2-methoxyethoxy)methyl)-3-azabicyclo[3.2.1]octane (153 mg, 1.9 equiv, HCl) in DMF (0.5 mL) was added DIEA (221 mg, 5.0 equiv) and 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (150 mg, 1.0 equiv) at 25° C. The reaction was stirred at 40° C. for 14 hours. The mixture was diluted with water (5 mL) and extracted with ethyl acetate (10 mL). The organic layer was dried over sodium sulfate and concentrated to afford the title compound (183 mg, crude) as yellow oil.

Step D. 5-ethyl-6-fluoro-4-(8-fluoro-2-(((2R,7aS)-2-fluorotetrahydro-1H-pyrrolizin-7a(5H)-yl)methoxy)-4-(1-((2-methoxyethoxy)methyl)-3-azabicyclo[3.2.1]octan-3-yl)pyrido[4,3-d]pyrimidin-7-yl)naphthalen-2-ol: To a solution of 7-chloro-8-fluoro-2-(((2R,7aS)-2-fluorotetrahydro-1H-pyrrolizin-7a(5H)-yl)methoxy)-4-(1-((2-methoxyethoxy)methyl)-3-azabicyclo[3.2.1]octan-3-yl)pyrido[4,3-d]pyrimidine (183 mg, 1.0 equiv) and 5-ethyl-6-fluoro-4-(4,4,5,5-tetramethyl-1,3,2-dioxaborolan-2-yl)naphthalen-2-ol (161 mg, 1.5 equiv) in methoxycyclopentane (10 mL) was added cataCXium A Pd G3 (49.5 mg, 0.2 equiv) and K₃PO₄ (1.5 M, 680 μL, 3.0 equiv). The mixture was degassed and purged with N₂ 3 times. The reaction was stirred at 80° C. for 3 hours under N₂ atmosphere. The mixture was diluted with water (20 mL) and extracted with ethyl acetate (50 mL). The organic layer was dried over sodium sulfate, concentrated and purified with column chromatography (SiO₂, Dichloromethane:Methanol=50/1 to 10/1) followed by prep-HPLC (column: Waters Xbridge 150×25 mm×5 um; mobile phase: A: water (NH₄HCO₃; B: ACN; B %: 60% to 90% over 8 min). to afford the title compound (105 mg, 44% yield) as yellow solid; SFC: column: Chiralcel OD-3 50× 4.6 mm I.D., 3 μm, mobile phase 60% EtOH (0.05% DEA) in CO2, flow rate: 3 mL/min, detector: 220 nm, tR₁: 0.511 min, tR₂: 1.603 min; ¹H NMR (400 MHZ, methanol-d4) δ=9.07 (d, J=3.2 Hz, 1H), 7.67 (dd, J=6.0, 9.2 Hz, 1H), 7.30 (d, J=2.8 Hz, 1H), 7.24 (t, J=9.6 Hz, 1H), 7.06 (s, 1H), 5.43-5.19 (m, 1H), 4.77-4.65 (m, 2H), 4.37-4.18 (m, 2H), 3.65-3.52 (m, 7H), 3.51-3.45 (m, 1H), 3.36 (s, 3H), 3.27-3.12 (m, 3H), 3.06-2.96 (m, 1H), 2.54-2.41 (m, 2H), 2.40-2.09 (m, 4H), 2.05-1.95 (m, 2H), 1.94-1.75 (m, 3H), 1.70-1.48 (m, 4H), 0.85-0.76 (m, 3H); LCMS (ESI, M+1): m/z=692.4.

### Example 858

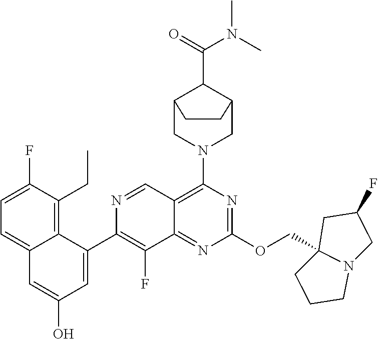 [SMILES: CCc1c(F)ccc2cc(O)cc(-c3ncc4c(N5CC6CCC(C5)C6C(=O)N(C)C)nc(OC[C@@]56CCCN5C[C@H](F)C6)nc4c3F)c12]

### (1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-3-azabicyclo[3.2.1]octane-8-carboxamide

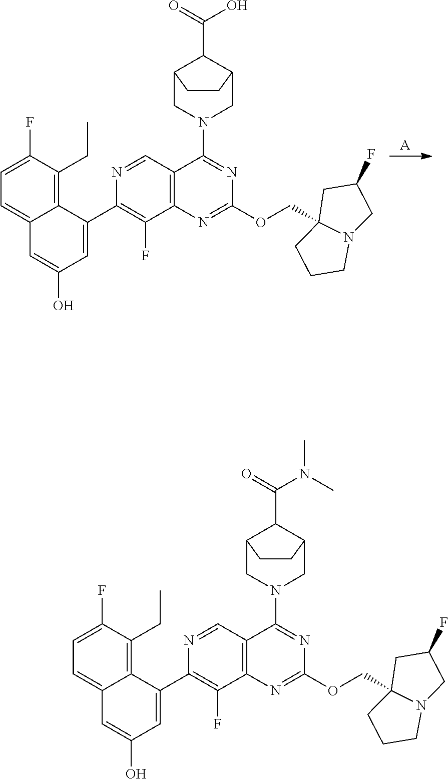

Step A. (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-N,N-dimethyl-3-azabicyclo[3.2.1]octane-8-carboxamide: To a solution of (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxylic acid (50.0 mg, 1.0 equiv), dimethylamine (6.96 mg, 2.0 equiv, 2M in THF) in DMF (0.5 mL) were added HATU (88.0 mg, 3.0 equiv), and DIEA. The reaction was stirred at 20° C. for 12 hours. The mixture was filtered and purified by prep-HPLC (column: Phenomenex luna C18 150×25 mm×10 um; mobile phase: [water (FA)-ACN]; B %: 15%-45%, 0 min). to afford the title compound (14.6 mg, 27% yield, 0.19 HCOOH) as yellow solid; ¹H NMR (400 MHZ, METHANOL-d4) δ=9.10 (s, 1H), 8.55-8.43 (m, 1H), 7.74-7.62 (m, 1H), 7.35-7.18 (m, 2H), 7.06 (br s, 1H), 5.54-5.31 (m, 1H), 4.81-4.73 (m, 2H), 4.53-4.36 (m, 2H), 3.85-3.71 (m, 2H), 3.67-3.42 (m, 3H), 3.22 (s, 5H), 2.94 (s, 3H), 2.64 (br s, 2H), 2.56-2.34 (m, 3H), 2.31-2.09 (m, 4H), 1.98 (br d, J=5.2 Hz, 3H), 1.57 (br d, J=8.4 Hz, 2H), 0.85-0.75 (m, 3H); LCMS (ESI, M+1): m/z=675.5.

### Example 859

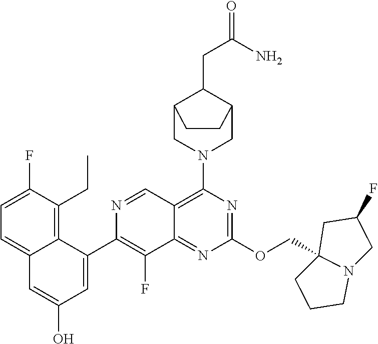 [SMILES: CCc1c(F)ccc2cc(O)cc(-c3ncc4c(N5CC6CCC(C5)C6CC(N)=O)nc(OC[C@@]56CCCN5C[C@H](F)C6)nc4c3F)c12]

### 2-((1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-yl)acetamide

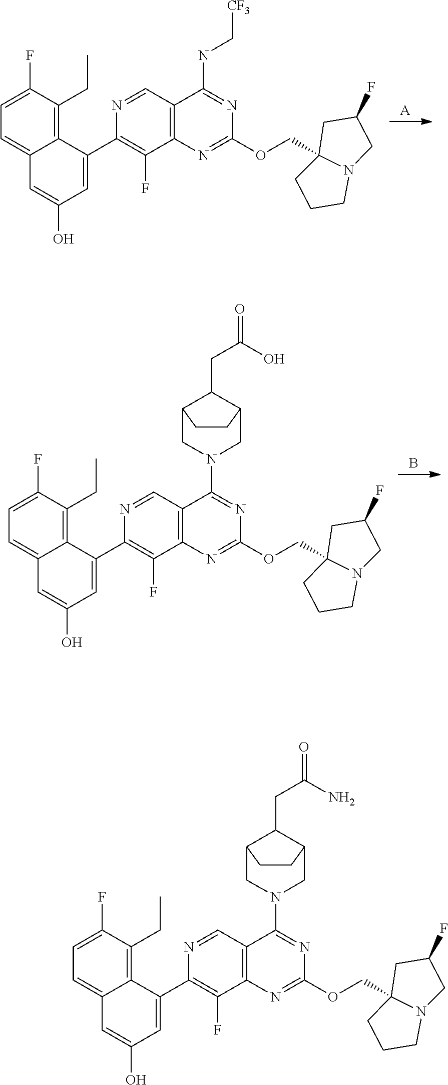

Step A. 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl) acetic acid: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (300 mg, 1.0 equiv) and 2-((1R,5S,8r)-3-azabicyclo[3.2.1]octan-8-yl) acetic acid (208 mg, 2.0 equiv, HCl) in DMF (0.1 mL) were added DIEA (196 mg, 3.0 equiv) and 4 Å molecular sieve (10 mg). The reaction was stirred at 60° C. for 12 hours. The mixture was filtered and purified by prep-HPLC (column: Waters xbridge 150*25 mm 10 um; mobile phase: [water (NH₄HCO₃)-ACN]; B %: 18%-48%, 11 min) to afford title compound (90.0 mg, 26% yield) as yellow solid. LCMS (ESI, M+1): m/z=662.6.

Step B. 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl)acetamide: To a mixture of 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl) acetic acid (50.0 mg, 1.0 equiv) and NH₄Cl (40.4 mg, 10 equiv) in DMF (0.3 mL) were added HATU (86.2 mg, 3.0 equiv) and DIEA (97.7 mg, 10 equiv). The reaction was stirred at 25° C. for 0.5 hour. The mixture was filtered and purified by prep-HPLC (column: Phenomenex luna C18 150*25 mm*10 um; mobile phase: [water (FA)-ACN]; B %: 15%-45%, 10 min) to afford the title compound (17.1 mg, 32% yield, 0.51 HCOOH) as yellow solid. ¹H NMR (400 MHZ, METHANOL-d₄) δ=9.29-9.05 (m, 1H), 7.68 (dd, J=5.6, 9.2 Hz, 1H), 7.41-7.19 (m, 2H), 7.06 (d, J=2.0 Hz, 1H), 5.62-5.35 (m, 1H), 4.65-4.44 (m, 5H), 3.98-3.78 (m, 2H), 3.76-3.63 (m, 3H), 2.75 (d, J=7.6 Hz, 1H), 2.67-2.54 (m, 1H), 2.52-2.45 (m, 2H), 2.40-2.15 (m, 8H), 2.13-2.02 (m, 1H), 1.93-1.81 (m, 2H), 1.68-1.52 (m, 2H), 0.79 (dt, J=2.8, 7.2 Hz, 3H); LCMS (ESI, M+1): m/z=661.0.

### Example 860

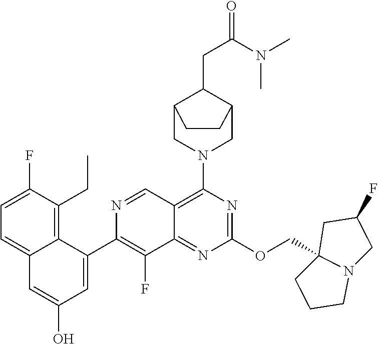 [SMILES: CCc1c(F)ccc2cc(O)cc(-c3ncc4c(N5CC6CCC(C5)C6CC(=O)N(C)C)nc(OC[C@@]56CCCN5C[C@H](F)C6)nc4c3F)c12]

### 2-((1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-yl)-N,N-dimethylacetamide

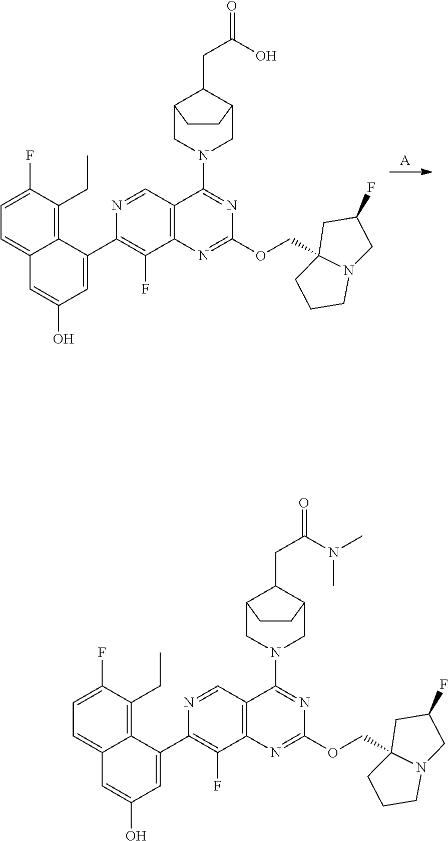

Step A. 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl)-N,N-dimethylacetamide: To a mixture of 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl) acetic acid (30.0 mg, 1.0 equiv) and dimethylamine (20.4 mg, 2M in THF) in DMF (0.1 mL) were added HATU (51.7 mg, 3.0 equiv) and DIEA (58.6 mg, 10 equiv). The reaction was stirred at 25° C. for 0.5 hour. The mixture was filtered and purified by prep-HPLC (column: Phenomenex luna C18 150*25 mm*10 um; mobile phase: [water (FA)-ACN]; B %: 18%-48%, 0 min) to afford the title compound (10.7 mg, 33% yield, 0.3 HCOOH) as yellow solid. ¹H NMR (400 MHZ, METHANOL-d4) δ=9.26-9.08 (m, 1H), 7.68 (dd, J=5.6, 9.2 Hz, 1H), 7.35-7.20 (m, 2H), 7.06 (d, J=2.4 Hz, 1H), 5.58-5.35 (m, 1H), 4.62-4.43 (m, 5H), 4.00-3.88 (m, 1H), 3.76 (br s, 3H), 3.21-3.06 (m, 4H), 3.00-2.91 (m, 4H), 2.65-2.41 (m, 3H), 2.41-2.12 (m, 8H), 2.12-1.99 (m, 1H), 1.94-1.80 (m, 2H), 1.68-1.51 (m, 2H), 0.79 (dt, J=2.8, 7.2 Hz, 3H); LCMS (ESI, M+1): m/z=689.7.

### Example 861

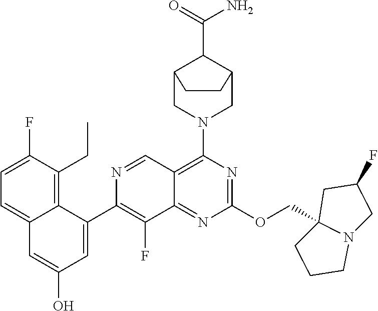 [SMILES: CCc1c(F)ccc2cc(O)cc(-c3ncc4c(N5CC6CCC(C5)C6C(N)=O)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxamide

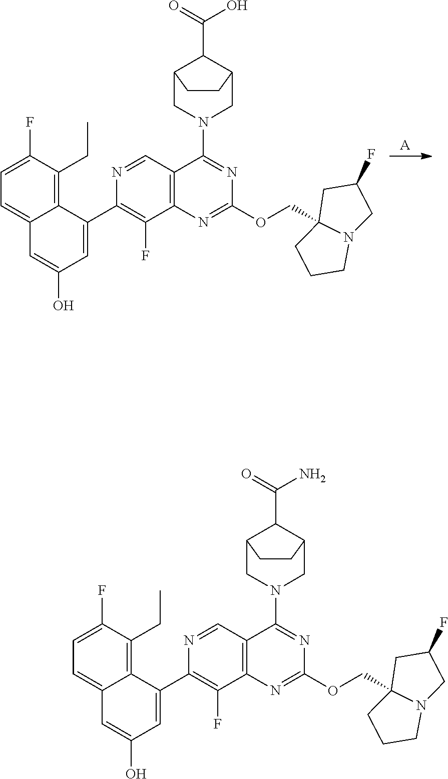

Step A. 3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxamide: To a solution of (1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-8-carboxylic acid (50 mg, 1 equiv) and DIEA (59.8 mg, 6 equiv) in DMF (0.5 mL) were added HATU (88 mg, 3 equiv) and NH₄Cl (6.19 mg, 1.5 equiv). The reaction was stirred at 20° C. for 0.5 hour. The mixture was filtered and purified by prep-HPLC (column: Phenomenex luna C18 150×25 mm×10 um; mobile phase: [water (FA)-ACN]; B %: 15%-45%, 10 min) to afford the title compound (16.1 mg, 30% yield) as white solid; ¹H NMR (400 MHZ, METHANOL-d4) δ=9.06 (s, 1H), 7.68 (dd, J=5.6, 8.8 Hz, 1H), 7.40-7.17 (m, 2H), 7.06 (d, J=2.4 Hz, 1H), 5.40-5.22 (m, 1H), 4.81-4.74 (m, 2H), 4.38-4.20 (m, 2H), 3.69 (br t, J=8.4 Hz, 2H), 3.27-3.17 (m, 2H), 3.10-2.96 (m, 1H), 2.85 (s, 1H), 2.73 (br s, 2H), 2.55-2.42 (m, 1H), 2.39-2.12 (m, 4H), 2.03-1.96 (m, 2H), 1.89 (br s, 2H), 1.57 (br d, J=8.8 Hz, 2H), 1.38-1.23 (m, 2H), 0.86-0.76 (m, 3H); LCMS (ESI, M+1): m/z=647.6.

### Example 862

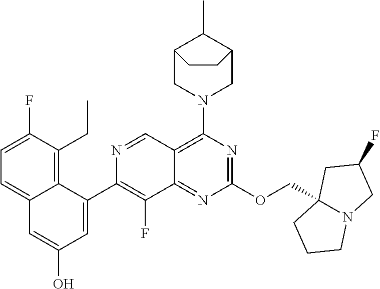 [SMILES: CCc1c(F)ccc2cc(O)cc(-c3ncc4c(N5CC6CCC(C5)C6C)nc(OC[C@@]56CCCN5C[C@H](F)C6)nc4c3F)c12]

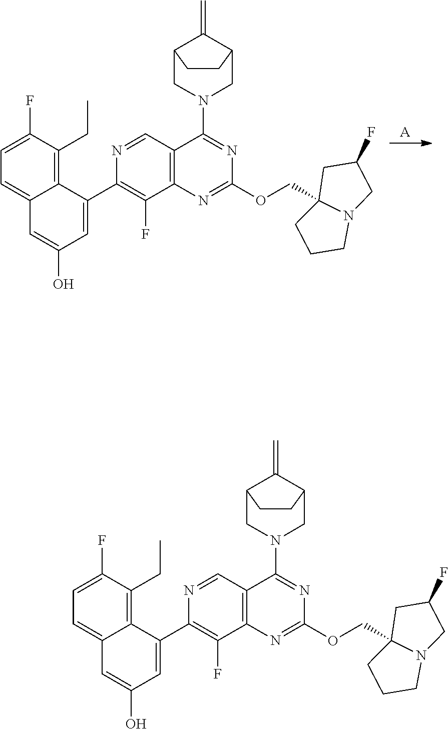

Step A. 5-ethyl-6-fluoro-4-(8-fluoro-2-(((2R,7aS)-2-fluorohexahydro-1H-pyrrolizin-7a-yl)methoxy)-4-((1R,5S,8S)-8-methyl-3-azabicyclo[3.2.1]octan-3-yl)pyrido[4,3-d]pyrimidin-7-yl)naphthalen-2-ol: To a solution of 5-ethyl-6-fluoro-4-(8-fluoro-2-(((2R,7aS)-2-fluorohexahydro-1H-pyrrolizin-7a-yl)methoxy)-4-((1R,5S)-8-methylene-3-azabicyclo[3.2.1]octan-3-yl)pyrido[4,3-d]pyrimidin-7-yl)naphthalen-2-ol (150 mg, 1 equiv) in MeOH (5 mL) was added Pd/C (10% purity, 10 mg). The reaction was degassed and purged with H₂ 3 times. The reaction was stirred at 20° C. for 10 min under H₂ at 15 psi. The mixture was filtered and purified by prep-HPLC (column: Welch Ultimate C18 150*25 mm*5 um; mobile phase: [water (FA)-ACN]; B %: 27%-57%, 10 min) to afford the title compound (54.4 mg, 35% yield, 0.2 HCOOH) as yellow solid. ¹H NMR (400 MHZ, DMSO-d6) δ=10.26-9.52 (m, 1H), 9.34-9.02 (m, 1H), 8.19 (s, 1H), 7.76 (dd, J=6.0, 9.2 Hz, 1H), 7.47-7.22 (m, 2H), 7.02 (d, J=2.0 Hz, 1H), 5.45-5.15 (m, 1H), 4.59-4.23 (m, 2H), 4.17-3.96 (m, 2H), 3.82 (br t, J=13.2 Hz, 1H), 3.60-3.53 (m, 1H), 3.16-2.97 (m, 4H), 2.88-2.76 (m, 1H), 2.42-2.28 (m, 1H), 2.19-1.98 (m, 6H), 1.91-1.66 (m, 6H), 1.54-1.25 (m, 4H), 0.72 (br t, J=7.2 Hz, 3H); LCMS (ESI, M+1): m/z=618.5.

### Example 863

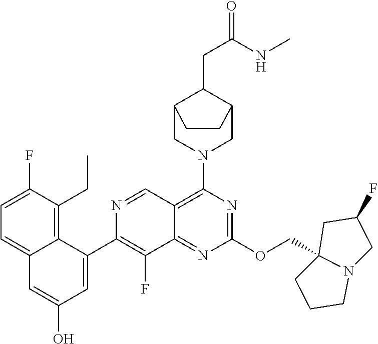 [SMILES: CCc1c(F)ccc2cc(O)cc(-c3ncc4c(N5CC6CCC(C5)C6CC(=O)NC)nc(OC[C@@]56CCCN5C[C@H](F)C6)nc4c3F)c12]

### 2-((1R,5S,8S)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-8-yl)-N-methylacetamide

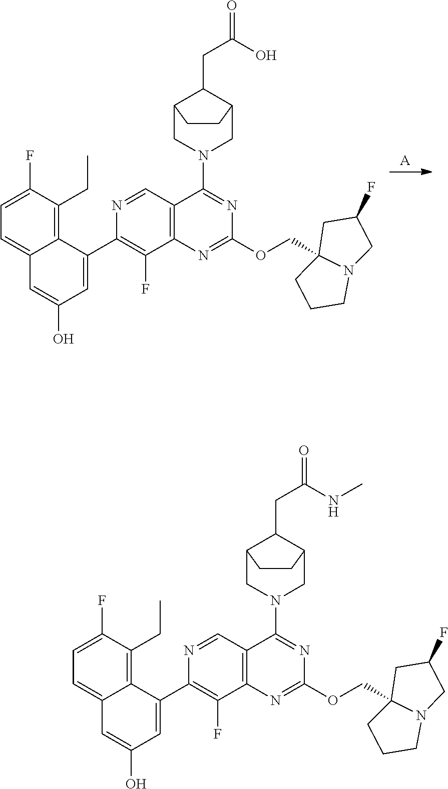

Step A. 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl)-N-methylacetamide: To a mixture of 2-((1R,5S,8S)-3-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octan-8-yl) acetic acid (60.0 mg, 1.0 equiv) and methanamine (61.2 mg, 10 equiv, HCl) in DMF (0.6 mL) were added HATU (345 mg, 10 equiv) and DIEA (35.2 mg, 3.0 equiv). The reaction was stirred at 25° C. for 0.5 hour. The mixture was filtered and purified by prep-HPLC (column: Phenomenex luna C18 150×25 mm×10 um; mobile phase: [water (FA)-ACN]; B %: 15%-45%, 10 min) to afford the title compound (5.70 mg, 9% yield, 0.38 HCOOH) as white solid. ¹H NMR (400 MHZ, METHANOL-d4) δ=9.20-9.08 (m, 1H), 7.68 (dd, J=6.0, 9.2 Hz, 1H), 7.36-7.16 (m, 2H), 7.06 (d, J=2.4 Hz, 1H), 5.55-5.36 (m, 1H), 4.55 (br s, 5H), 3.95-3.89 (m, 1H), 3.69-3.53 (m, 3H), 2.81-2.74 (m, 3H), 2.72 (br d, J=7.6 Hz, 2H), 2.61-2.41 (m, 3H), 2.34-2.15 (m, 7H), 2.14-1.98 (m, 2H), 1.88 (br d, J=6.0 Hz, 2H), 1.63 (br d, J=8.4 Hz, 2H), 0.87-0.72 (m, 3H); LCMS (ESI, M+1): m/z=675.5.

### Example 864

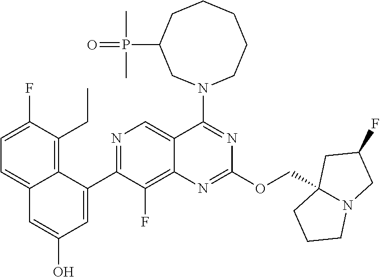 [SMILES: CCc1c(F)ccc2cc(O)cc(-c3ncc4c(N5CCCCCC(P(C)(C)=O)C5)nc(OC[C@@]56CCCN5C[C@H](F)C6)nc4c3F)c12]

### (1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)azocan-3-yl)dimethylphosphine oxide

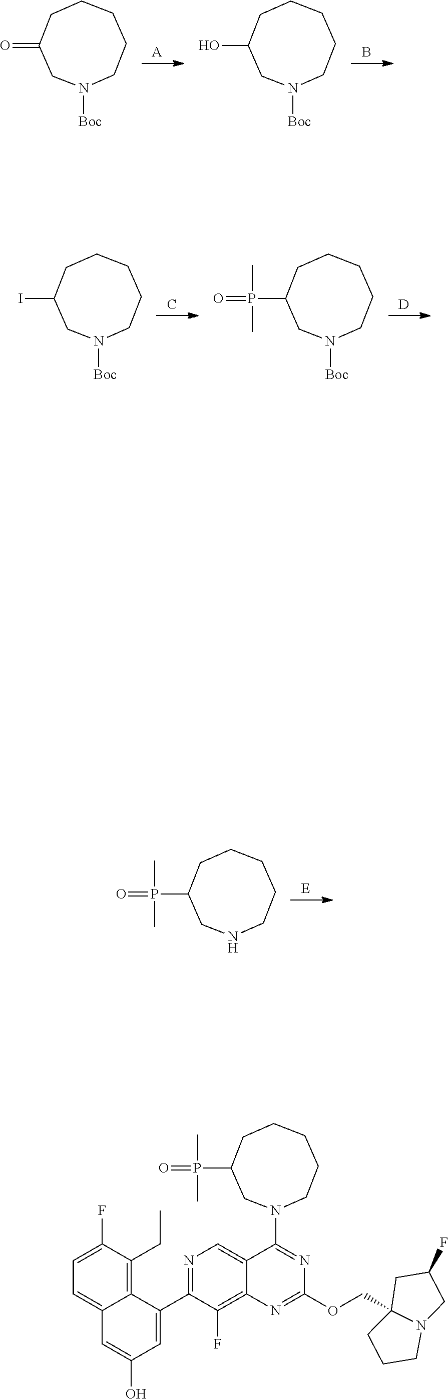

Step A. tert-butyl 3-hydroxyazocane-1-carboxylate: To a solution of tert-butyl 3-oxoazocane-1-carboxylate (800 mg, 1.0 equiv) in MeOH (8 mL) was added NaBH₄ (266 mg, 2.0 equiv) at 0° C. slowly. The reaction was stirred at 0° C. for 2 hours. The mixture was quenched with saturated NH₄Cl (5 mL), concentrated and extracted with ethyl acetate (3×3 mL). The combined organic layers were dried over sodium sulfate, filtered and concentrated to afford the title compound (800 mg, crude) as a yellow oil. ¹H NMR (400 MHZ, CHLOROFORM-d) δ=3.99-3.81 (m, 1H), 3.68-3.50 (m, 1H), 3.49-3.13 (m, 3H), 2.03-1.80 (m, 3H), 1.79-1.56 (m, 5H), 1.49 (s, 9H).

Step B. tert-butyl 3-iodoazocane-1-carboxylate: To a solution of tert-butyl 3-hydroxyazocane-1-carboxylate (800 mg, 1.0 equiv), imidazole (356 mg, 1.5 equiv) and PPh₃ (1.37 g, 1.5 equiv) in DCM (16 mL) was added I₂ (1.33 g, 1.5 equiv) in portions. The reaction was stirred at 25° C. for 5 hours. The mixture was concentrated and purified by flash silica gel chromatography (ISCO®; 12 g SepaFlash® Silica Flash Column, Eluent of 0˜25% Ethyl acetate/Petroleum ethergradient@15 mL/min) to afford the title compound (750 mg, 63% yield) as a yellow oil. ¹H NMR (400 MHZ, CHLOROFORM-d) δ=4.70-4.48 (m, 1H), 3.63-3.13 (m, 4H), 2.52-2.11 (m, 4H), 1.82-1.63 (m, 4H), 1.50 (d, J=11.7 Hz, 9H).

Step C. tert-butyl 3-(dimethylphosphoryl) azocane-1-carboxylate: To a solution of methylphosphonoylmethane (345 mg. 2.0 equiv) in THF (3 mL) was added NaHMDS (1 M, 4.42 mL, 2.0 equiv) dropwise at 0° C. The mixture was stirred at 0° C. for 1 hour. tert-butyl 3-iodoazocane-1-carboxylate (750 mg, 1.0 equiv) was added. The reaction was stirred at 20° C. for 11 hours. The mixture was concentrated and purified by prep-HPLC [column: Phenomenex luna C18 150×25 mm×10 μm; A: water (FA), B: ACN, B %: 18%-48% over 10 min] to afford the title compound (176 mg, 28% yield) as a yellow oil. ¹H NMR (400 MHZ, CHLOROFORM-d) δ=3.68-3.49 (m, 1H), 3.44-3.36 (m, 1H), 3.35-3.24 (m, 2H), 2.19-1.89 (m, 2H), 1.77-1.57 (m, 7H), 1.47 (s, 12H), 1.45-1.41 (m, 3H).

Step D. azocan-3-yldimethylphosphine oxide: To a solution of tert-butyl 3-(dimethylphosphoryl) azocane-1-carboxylate (160 mg, 1.0 equiv) in dioxane (0.8 mL) was added HCl/dioxane (4 M, 800 μL, 5.79 equiv). The reaction was stirred at 25° C. for 2 hours. The mixture was concentrated to afford the title compound (120 mg, crude, HCl salt) as a yellow oil.

Step E. (1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl) azocan-3-yl)dimethylphosphine oxide: A mixture of azocan-3-yldimethylphosphine oxide (50.0 mg, 1.0 equiv, HCl salt), 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (84.0 mg, 0.8 equiv), K₃PO₄ (113 mg, 3.0 equiv) and 4 Å molecular sieve (50 mg) in DMF (0.5 mL) was degassed and purged with N₂ for 3 times. The reaction was stirred at 60° C. for 2 hours under N₂ atmosphere. The mixture was filtered, and then the filtrate was concentrated and purified by prep-HPLC [column: Waters Xbridge 150×25 mm×5 μm; A: water (ammonia hydroxide v/v), B: ACN, B %: 30%-60% over 9 min] to afford the title compound (21.0 mg, 17% yield) as a yellow solid. ¹H NMR (400 MHZ, METHANOL-d₄) δ=9.13 (br d, J=5.4 Hz, 1H), 7.67 (br dd, J=6.1, 8.7 Hz, 1H), 7.30 (br s, 1H), 7.25 (br t, J=9.3 Hz, 1H), 7.05 (br d, J=12.2 Hz, 1H), 5.39-5.21 (m, 1H), 4.42-4.17 (m, 4H), 4.17-3.98 (m, 2H), 3.29-3.12 (m, 3H), 3.07-2.95 (m, 1H), 2.48 (td, J=7.1, 14.5 Hz, 2H), 2.37-2.10 (m, 5H), 2.09-1.93 (m, 6H), 1.86 (br d, J=1.1 Hz, 3H), 1.70-1.56 (m, 1H), 1.50 (br d, J=12.3 Hz, 6H), 0.80 (br d, J=6.5 Hz, 3H); LCMS (ESI, M+1): m/z=682.5.

### Example 865

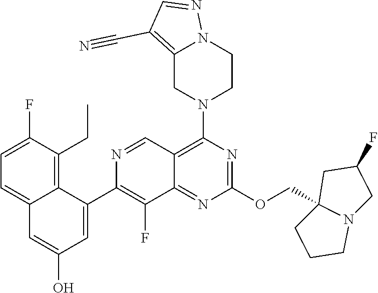 [SMILES: CCc1c(F)ccc2cc(O)cc(-c3ncc4c(N5CCn6ncc(C#N)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,5,6,7-tetrahydropyrazolo[1,5-a]pyrazine-3-carbonitrile

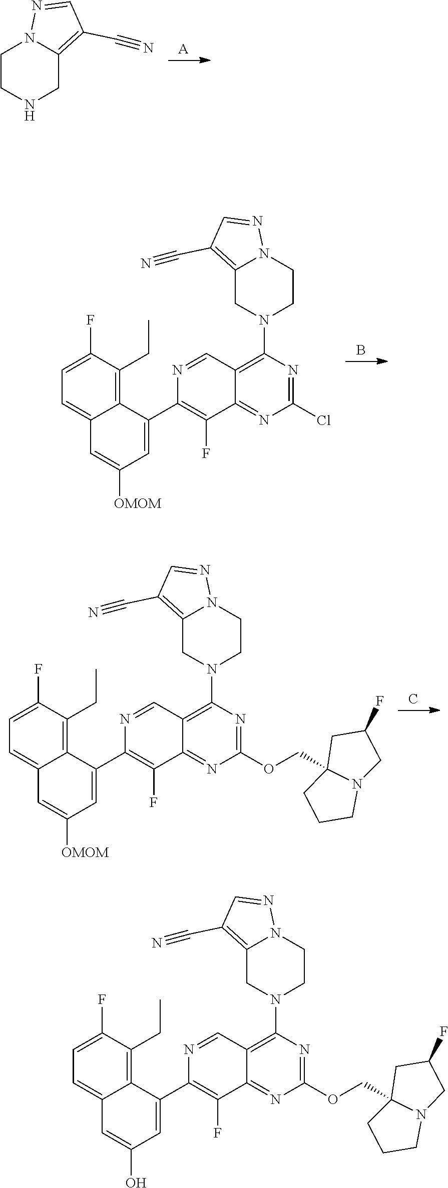

Step A. 5-(2-chloro-7-(8-ethyl-7-fluoro-3-(methoxymethoxy)naphthalen-1-yl)-8-fluoropyrido[4,3-d]pyrimidin-4-yl)-4,5,6,7-tetrahydropyrazolo[1,5-a]pyrazine-3-carbonitrile: To a mixture of 2,4-dichloro-7-(8-ethyl-7-fluoro-3-(methoxymethoxy)naphthalen-1-yl)-8-fluoropyrido[4,3-d]pyrimidine (45.6 mg, 1.0 equiv) and 4 Å molecular sieve (10.0 mg, 1.0 equiv) in DMF (1 mL) was added DIEA (39.2 mg, 3.0 equiv) at −40° C. The reaction mixture was stirred for 5 minutes. Then 4,5,6,7-tetrahydropyrazolo[1,5-a]pyrazine-3-carbonitrile (15.0 mg, 1.0 equiv) was added to the mixture and stirred at −40° C. for 30 minutes. The mixture was diluted with H₂O (30 mL) and extracted with ethyl acetate (2×30 mL), washed with brine (10 mL), dried, concentrated and purified with prep-TLC [SiO₂, dichloromethane/methanol=10/1] to afford the title compound (40.0 mg, 70% yield) as a white solid; LCMS (ESI, M+1): m/z=562.3

Step B. 5-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-4,5,6,7-tetrahydropyrazolo[1,5-a]pyrazine-3-carbonitrile: To a mixture of 5-(2-chloro-7-(8-ethyl-7-fluoro-3-(methoxymethoxy)naphthalen-1-yl)-8-fluoropyrido[4,3-d]pyrimidin-4-yl)-4,5,6,7-tetrahydropyrazolo[1,5-a]pyrazine-3-carbonitrile (40.0 mg, 1.0 equiv), ((2R,7aS)-2-fluorotetrahydro-1H-pyrrolizin-7a(5H)-yl)methanol (28.3 mg, 2.5 equiv) and 4 Å molecular sieve (10.0 mg, 1.0 equiv) in DMF (1 mL) was added DIEA (27.6 mg, 3.0 equiv). The reaction mixture was stirred at 60° C. for 12 hours. The mixture was diluted with H₂O (30 mL) and extracted with ethyl acetate (2× 30 mL), washed with brine (10 mL), dried, concentrated and purified with prep-TLC [SiO₂, dichloromethane/methanol=10/1] to afford the title compound (30.0 mg, 61% yield) as a white solid; LCMS (ESI, M+1): m/z=685.4.

Step C. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4,5,6,7-tetrahydropyrazolo[1,5-a]pyrazine-3-carbonitrile: To a solution of 5-(7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)pyrido[4,3-d]pyrimidin-4-yl)-4,5,6,7-tetrahydropyrazolo[1,5-a]pyrazine-3-carbonitrile (30 mg, 1.0 equiv) in MeCN (1 mL) was added HCl·dioxane (4M, 1 mL). The reaction mixture was stirred at 0° C. for 30 minutes. The mixture was adjusted with NaHCO₃ to PH˜7. The mixture was filtered and purified with prep-HPLC [column: Waters Xbridge 150×25 mm×5 um; mobile phase: [water (ammonia hydroxide v/v)-ACN]; B %: 76%-76%, 9 min] to afford the title compound (4.95 mg, 20% yield) as off-white solid; ¹H NMR (400 MHZ, METHANOL-d4) δ=9.22 (s, 1H), 7.95 (s, 1H), 7.66 (br dd, J=5.2, 8.8 Hz, 1H), 7.33-7.19 (m, 2H), 7.04 (br d, J=1.2 Hz, 1H), 5.42-5.22 (m, 3H), 4.54 (br d, J=3.2 Hz, 4H), 4.35 (q, J=10.8 Hz, 2H), 3.19 (s, 2H), 3.09-2.95 (m, 1H), 2.52-1.85 (m, 9H), 0.85-0.74 (m, 3H); LCMS (ESI, M+1): m/z=641.4.

### Example 866

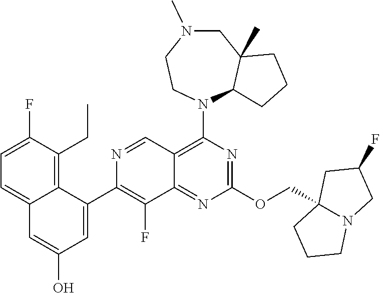 [SMILES: CCc1c(F)ccc2cc(O)cc(-c3ncc4c(N5CCN(C)C[C@]6(C)CCC[C@@H]56)nc(OC[C@@]56CCCN5C[C@H](F)C6)nc4c3F)c12]

### 4-(4-((5aS,8aR)-4,5a-dimethyloctahydrocyclopenta[e][1,4]diazepin-1(2H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

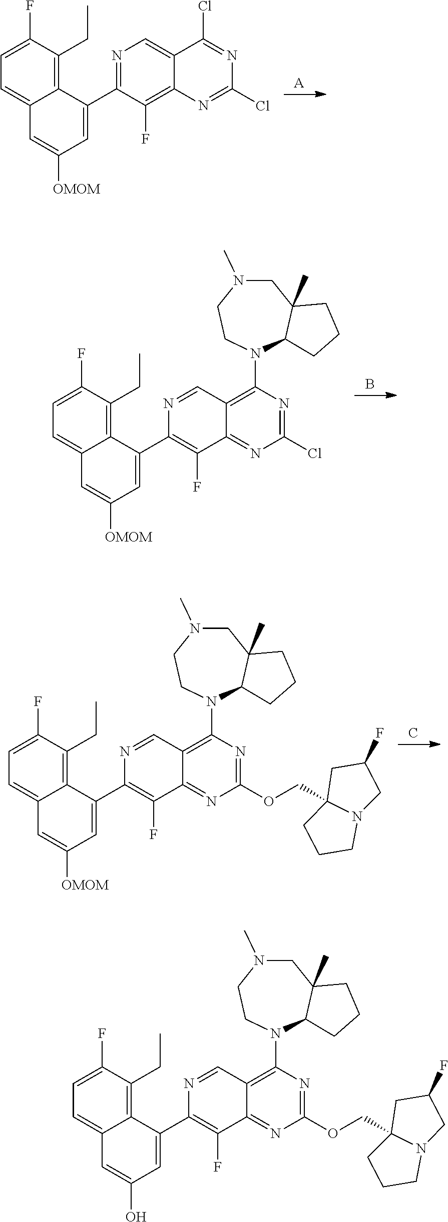

Step A. 2-chloro-4-((5aS,8aR)-4,5a-dimethyloctahydrocyclopenta[e][1,4]diazepin-1(2H)-yl)-7-(8-ethyl-7-fluoro-3-(methoxymethoxy)naphthalen-1-yl)-8-fluoropyrido[4,3-d]pyrimidine: To a solution of 2,4-dichloro-7-(8-ethyl-7-fluoro-3-(methoxymethoxy)naphthalen-1-yl)-8-fluoropyrido[4,3-d]pyrimidine (64.2 mg, 0.8 equiv) and (5aS,8aR)-4,5a-dimethyldecahydrocyclopenta[e][1,4]diazepine (30.0 mg, 1.0 equiv) in ACN (2.0 mL) were added DIEA (69.1 mg, 3.0 equiv) and 4 Å molecular sieve (10.0 mg). The mixture was stirred at 0° C. for 0.5 hours. The mixture was filtered and extracted with ethyl acetate (2×30 mL). The combined organic layers were washed with brine (10 mL), dried over sodium sulfate, concentrated and purified with prep-HPLC [column: Phenomenex Luna C18 150×25 mm×10 um; mobile phase: [water (FA)-ACN]; B %: 22%-52%, 9 min] to afford the title compound (20.0 mg, 19% yield) as a white solid.

Step B. 4-((5aS,8aR)-4,5a-dimethyloctahydrocyclopenta[e][1,4]diazepin-1(2H)-yl)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine: To a solution of 2-chloro-4-((5aS,8aR)-4,5a-dimethyloctahydrocyclopenta[e][1,4]diazepin-1(2H)-yl)-7-(8-ethyl-7-fluoro-3-(methoxymethoxy)naphthalen-1-yl)-8-fluoropyrido[4,3-d]pyrimidine (20.0 mg, 1.0 equiv) and ((2R,7aS)-2-fluorotetrahydro-1H-pyrrolizin-7a(5H)-yl)methanol (16.4 mg, 3.0 equiv) in THF (2 mL) were added DIEA (22.2 mg, 5.0 equiv) and 4 Å molecular sieve (20.0 mg). The mixture was stirred at 60° C. for 12 hours. The mixture was filtered and extracted with ethyl acetate (2×30 mL). The combined organic layers were washed with brine (10 mL), dried, concentrated and purified by prep-HPLC [column: Waters Xbridge 150×25 mm×5 um, mobile phase: [water (ammonia hydroxide v/v)-ACN]; B %: 75%-100%, 9 min] to afford the title compound (10 mg, 41% yield) as a white solid.

Step C. 4-(4-((5aS,8aR)-4,5a-dimethyloctahydrocyclopenta[e][1,4]diazepin-1(2H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a solution of 4-((5aS,8aR)-4,5a-dimethyloctahydrocyclopenta[e][1,4]diazepin-1(2H)-yl)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidine (10.0 mg, 1.0 equiv) in ACN (2.4 mL) was added HCl·dioxane (4 M, 0.5 mL). The mixture was stirred at 0° C. for 0.5 hours. The mixture was quenched with saturated NaHCO₃ aqueous solution (20 mL), extracted with ethyl acetate (3×10 mL), dried, concentrated and purified with prep-HPLC [column: Waters Xbridge 150×25 mm×5 um; mobile phase: [water (ammonia hydroxide v/v)-ACN]; B %: 60%-90%, 9 min] to afford the title compound (2.50 mg, 26% yield, 96% purity) as a white solid; ¹H NMR (400 MHZ, METHANOL-d₄) δ=9.07 (d, J=2.4 Hz, 1H), 7.69 (dd, J=5.6, 8.8 Hz, 1H), 7.32 (d, J=2.4 Hz, 1H), 7.26 (t, J=9.6 Hz, 1H), 7.04 (dd, J=2.4, 19.6 Hz, 1H), 5.60-5.42 (m, 1H), 4.74-4.50 (m, 8H), 4.32-4.26 (m, 1H), 3.79-3.65 (m, 2H), 3.23-2.92 (m, 3H), 2.57-2.23 (m, 10H), 1.91-1.65 (m, 3H), 1.46 (d, J=4.4 Hz, 4H), 1.36-1.22 (m, 2H), 0.85-0.74 (m, 3H); LCMS (ESI, M+1): m/z=661.5.

### Example 867

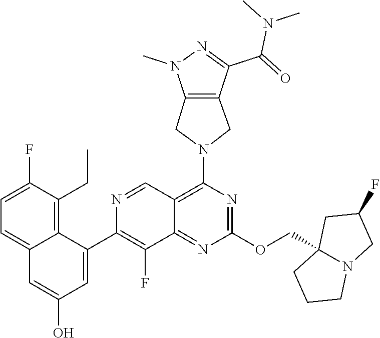 [SMILES: CCc1c(F)ccc2cc(O)cc(-c3ncc4c(N5Cc6c(C(=O)N(C)C)nn(C)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N, 1-trimethyl-1,4,5,6-tetrahydropyrrolo[3,4-c]pyrazole-3-carboxamide

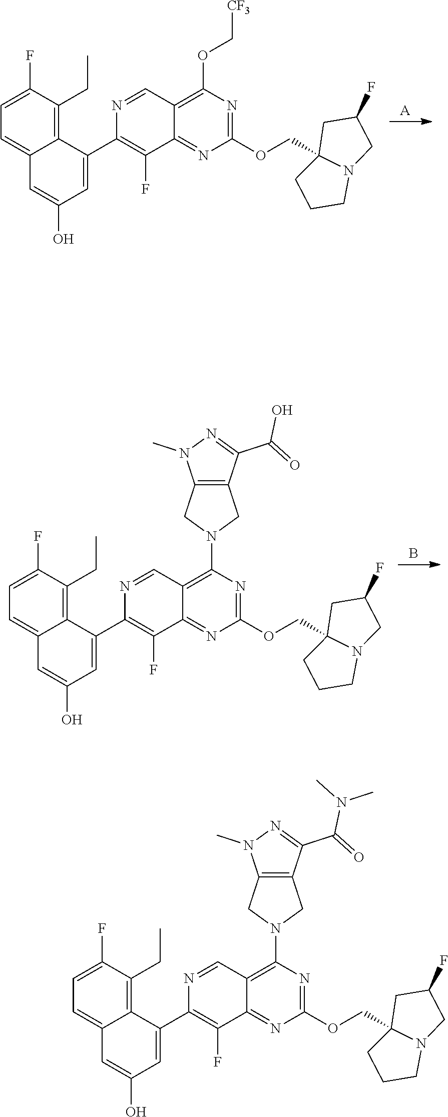

Step A. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-methyl-1,4,5,6-tetrahydropyrrolo[3,4-c]pyrazole-3-carboxylic acid: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (255 mg, 0.6 equiv) and methyl 1-methyl-5,6-dihydro-4H-pyrrolo[3,4-c]pyrazole-3-carboxylate (130 mg, 1.0 equiv) in DMF (2.0 mL) were added K₃PO₄ (1.52 g, 10 equiv) and 4 Å molecular sieve (100 mg, 10 equiv). The reaction was stirred at 60° C. for 2 hours. The mixture was filtered and extracted with ethyl acetate (2×30 mL). The combined organic layers were washed with brine (10 mL), dried, concentrated and purified with prep-HPLC [column: Phenomenex luna C18 150×25 mm×10 um; mobile phase: [water (FA)-ACN]; B %: 17%-47%, 10 min] to afford the title compound (50.0 mg, 10% yield) as a white solid.

Step B. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N,N,1-trimethyl-1,4,5,6-tetrahydropyrrolo[3,4-c]pyrazole-3-carboxamide: To a solution of 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-1-methyl-1,4,5,6-tetrahydropyrrolo[3,4-c]pyrazole-3-carboxylic acid (20.0 mg, 1.0 equiv) in DMF (1 mL) were added HATU (17.3 mg, 1.5 equiv) and TEA (9.20 mg, 3.0 equiv). The mixture was stirred at 20° C. for 2 hours. Then to the reaction was added dimethylamine (2 M, 75.8 μL) and the mixture was stirred at 20° C. for 2 hours. The mixture was filtered and extracted with ethyl acetate (2×30 mL). The combined organic layers were washed with brine (10 mL), dried, concentrated and purified by prep-HPLC [column: 3_Phenomenex Luna C18 75×30 mm×3 um; mobile phase: [water (FA)-ACN]; B %: 21%-41%, 8 min] to afford the title compound (4 mg, 19% yield) as a white solid; ¹H NMR (400 MHZ, METHANOL-d₄) δ=11.54 (br t, J=7.2 Hz, 3H), 10.28-10.12 (m, 4H), 10.09-9.83 (m, 4H), 9.19 (br s, 4H), 8.89-8.75 (m, 6H), 8.46-8.26 (m, 3H), 7.81-7.64 (m, 4H), 7.01-6.76 (m, 3H), 5.37-5.20 (m, 1H), 5.14-4.95 (m, 2H), 4.66 (dd, J=6.0, 8.8 Hz, 1H), 3.90-3.76 (m, 1H), 2.87 (br s, 1H); LCMS (ESI, M+1): m/z=687.5.

### Example 868

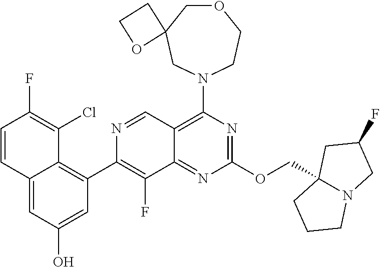 [SMILES: Oc1cc(-c2ncc3c(N4CCOCC5(CCO5)C4)nc(OC[C@@]45CCCN4C[C@H](F)C5)nc3c2F)c2c(Cl)c(F)ccc2c1]

### 5-chloro-6-fluoro-4-(8-fluoro-2-(((2R,7aS)-2-fluorotetrahydro-1H-pyrrolizin-7a(5H)-yl)methoxy)-4-(1,6-dioxa-9-azaspiro[3.6]decan-9-yl)pyrido[4,3-d]pyrimidin-7-yl)naphthalen-2-ol

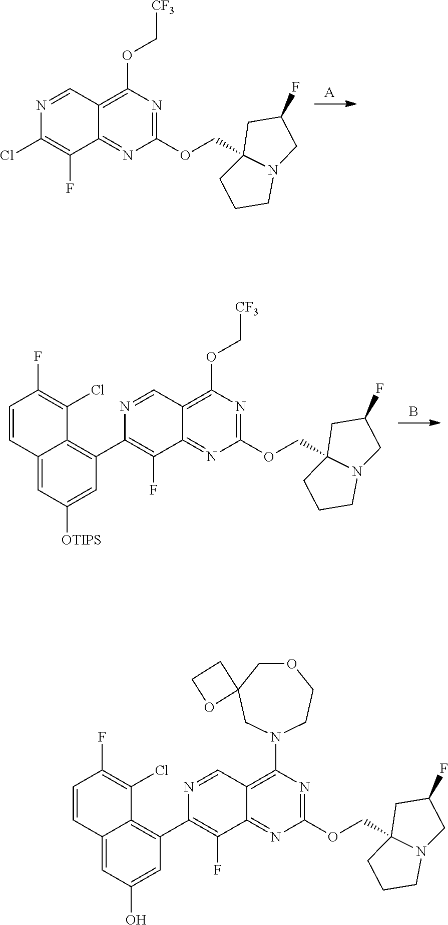

Step A. 7-(8-chloro-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (200 mg, 1.0 equiv), CuI (26.0 mg, 0.3 equiv) and ((5-chloro-6-fluoro-4-(trimethylstannyl)naphthalen-2-yl)oxy)triisopropylsilane (353 mg, 1.5 equiv) in toluene (3 mL) were added BINAP (56.8 mg, 0.2 equiv) and [1,1-Bis(diphenylphosphino)ferrocene]dichloropalladium(II) (33.4 mg, 0.1 equiv). The reaction was stirred at 90° C. for 16 hours under N₂. The mixture was diluted with water (10 mL) and extracted with ethyl acetate (2×10 mL). The combined organic layers were dried over anhydrous sodium sulfate, filtered, concentrated and purified by reversed phase flash chromatography [Phenomenex luna C18 150×25 mm×10 um; water (0.1% FA)/acetonitrile] to afford the title compound (171 mg, 46.2% yield) as yellow oil; ¹H NMR (400 MHZ, CHLOROFORM-d) δ=9.25 (s, 1H), 7.70 (dd, J=5.6, 8.8 Hz, 1H), 7.37-7.30 (m, 2H), 7.27 (s, 2H), 7.24 (s, 1H), 5.42-5.21 (m, 1H), 5.17-4.97 (m, 2H), 3.43-3.13 (m, 3H), 3.07-2.95 (m, 1H), 2.36-2.09 (m, 3H), 2.03-1.89 (m, 3H), 1.38 (br s, 3H), 1.13 (d, J=7.4 Hz, 18H); LCMS (ESI, M+1): m/z=755.3.

Step B. 5-chloro-6-fluoro-4-(8-fluoro-2-(((2R,7aS)-2-fluorotetrahydro-1H-pyrrolizin-7a(5H)-yl)methoxy)-4-(1,6-dioxa-9-azaspiro[3.6]decan-9-yl)pyrido[4,3-d]pyrimidin-7-yl)naphthalen-2-ol: To a mixture of 7-(8-chloro-7-fluoro-3-((triisopropylsilyl)oxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (80.0 mg, 1.0 equiv) and 1,6-dioxa-9-azaspiro[3.6]decane (38.1 mg, 2.0 equiv, HCl) in DMF (1 mL) were added K₃PO₄ (112 mg, 5.0 equiv) and 4 Å molecular sieve (80 mg). The reaction was stirred at 100° C. for 7 hours. The mixture was filtered and the filtrate was purified by prep-HPLC [column: Phenomenex Luna C18 150×25 mm×10 um; mobile phase: water (FA)-ACN; B %: 13%-43%, 9 min] to afford the title compound (7.9 mg, 11% yield, HCOOH) as yellow solid; ¹H NMR (400 MHZ, METHANOL-d4) δ=9.65-9.52 (m, 1H), 7.88-7.79 (m, 1H), 7.48-7.34 (m, 2H), 7.24 (dd, J=2.4, 9.6 Hz, 1H), 5.58-5.35 (m, 1H), 4.71-4.36 (m, 6H), 4.30-4.15 (m, 1H), 4.12 (br s, 4H), 3.95-3.80 (m, 1H), 3.77-3.45 (m, 4H), 2.79-2.67 (m, 1H), 2.63-2.36 (m, 3H), 2.29 (br d, J=8.8 Hz, 1H), 2.24-2.14 (m, 2H), 2.12-1.96 (m, 1H); LCMS (ESI, M+1): m/z=642.6.

### Example 869

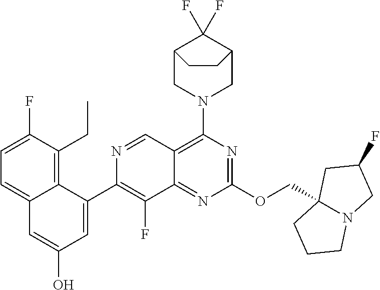 [SMILES: CCc1c(F)ccc2cc(O)cc(-c3ncc4c(N5CC6CCC(C5)C6(F)F)nc(OC[C@@]56CCCN5C[C@H](F)C6)nc4c3F)c12]

### 4-(4-(8,8-difluoro-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

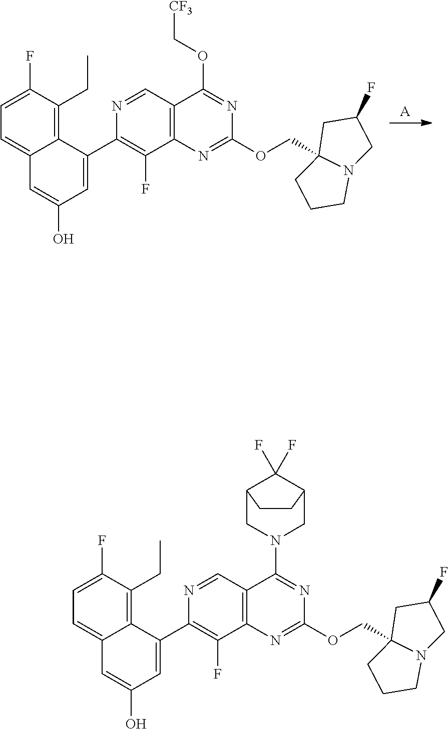

Step A. 4-(4-(8,8-difluoro-3-azabicyclo[3.2.1]octan-3-yl)-8-fluoro-2-(((2R,7aS)-2-fluorohexahydro-1H-pyrrolizin-7a-yl)methoxy)pyrido[4,3-d]pyrimidin-7-yl)-5-ethyl-6-fluoronaphthalen-2-ol: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (50.0 mg, 1.0 equiv) and 8,8-difluoro-3-azabicyclo[3.2.1]octane (31.0 mg, 2.0 equiv, HCl) in DMF (0.2 mL) were added DIEA (109 mg, 10 equiv) and 4 Å molecular sieve (5.00 mg, 1.0 equiv). The reaction was stirred at 40° C. for 12 hours. The mixture was filtered and purified by reversed-phase HPLC (column: Phenomenex luna C18 150×25 mm×10 um; mobile phase: [water (FA)-ACN]; B %: 22%-52%, 10 min) and purified by reversed-phase HPLC (column: Welch Xtimate C18 150×25 mm×5 um; mobile phase: [water(NH₃H₂O)-ACN]; B %: 45%-75%, 8 min) to afford the title compound (3.67 mg, 6.7% yield) as blue solid; ¹HNMR (400 MHz, CHLOROFORM-d) δ=9.06-8.92 (m, 1H), 7.65-7.44 (m, 1H), 7.25-7.17 (m, 2H), 7.12-6.93 (m, 1H), 5.45-5.18 (m, 1H), 4.77-4.61 (m, 2H), 4.33-4.25 (m, 1H), 3.90-3.72 (m, 2H), 3.41-3.17 (m, 2H), 3.09-2.95 (m, 1H), 2.52-2.33 (m, 3H), 2.31-2.11 (m, 3H), 2.06-1.89 (m, 5H), 1.34-1.26 (m, 4H), 0.92-0.78 (m, 4H); LCMS (ESI, M+1): m/z=640.0.

### Example 870

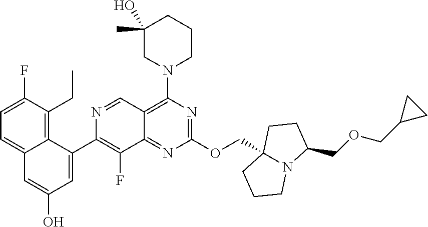 [SMILES: CCc1c(F)ccc2cc(O)cc(-c3ncc4c(N5CCC[C@@](C)(O)C5)nc(OC[C@@]56CCCN5[C@H](COCC5CC5)CC6)nc4c3F)c12]

### (R)-1-(2-(((3S,7aS)-3-((cyclopropylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

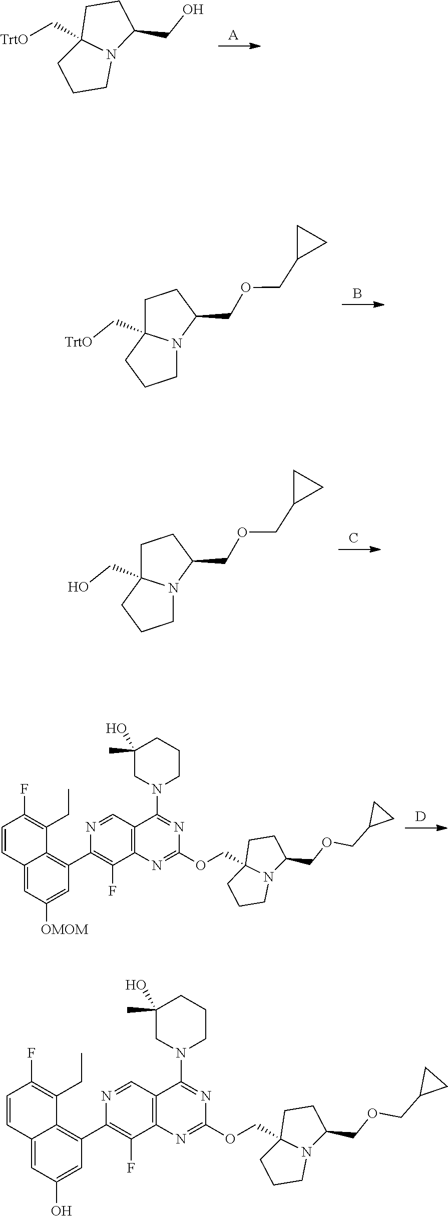

Step A. (3S,7aS)-3-((cyclopropylmethoxy)methyl)-7a-((trityloxy)methyl) hexahydro-1H-pyrrolizine: To a mixture of ((3S,7aS)-7a-((trityloxy)methyl) hexahydro-1H-pyrrolizin-3-yl)methanol (500 mg, 1.0 equiv) in THF (2 mL) and DMF (2 mL) was added NaH (96.7 mg, 60% purity, 2.0 equiv). The reaction was stirred at 0-25° C. for 0.5 hours. To the mixture was added (iodomethyl)cyclopropane (330 mg, 1.5 equiv). The reaction was stirred at 25° C. for 11.5 hours. The mixture was quenched with H₂O (5 mL) and extracted with ethyl acetate (2×10 mL). The combined organic layers were dried over Na₂SO₄, filtered, concentrated and purified by reversed phase flash chromatography [Phenomenex luna C18 150×25 mm×10 um; water (0.1% FA)/acetonitrile] to afford the title compound (415 mg, 71% yield) as yellow oil; ¹H NMR (400 MHZ, CHLOROFORM-d) δ=7.27 (s, 5H), 7.14-7.06 (m, 7H), 7.06-6.99 (m, 3H), 3.50 (dd, J=6.4, 9.6 Hz, 1H), 3.35 (br s, 1H), 3.09 (d, J=6.8 Hz, 2H), 3.05-2.82 (m, 2H), 2.73 (br d, J=6.0 Hz, 2H), 2.48 (br s, 1H), 1.93 (br dd, J=6.8, 12.0 Hz, 1H), 1.65-1.56 (m, 2H), 1.53 (br d, J=5.6 Hz, 2H), 1.43 (br s, 2H), 1.32-1.22 (m, 1H), 0.91-0.79 (m, 1H), 0.37-0.28 (m, 2H), 0.01 (q, J=4.8 Hz, 2H); LCMS (ESI, M+1): m/z=468.4.

Step B. ((3S,7aS)-3-((cyclopropylmethoxy)methyl)tetrahydro-1H-pyrrolizin-7a(5H)-yl)methanol: To a mixture of (3S,7aS)-3-((cyclopropylmethoxy)methyl)-7a-((trityloxy)methyl) hexahydro-1H-pyrrolizine (415 mg, 1.0 equiv) in DCM (4 mL) was added TFA (1.01 g, 10 equiv). The reaction was stirred at 0-25° C. for 12 hours. The mixture was concentrated, dissolved in MeOH (2 mL), neutralized with solid NaHCO₃ and filtered. The filtrate was concentrated and purified by column chromatography (Al₂O₃, Petroleum ether:Ethyl acetate=5:1 to 0:1 to Dichloromethane:Methanol=20:1) to afford the title compound (210 mg, 95% yield) as yellow oil; ¹H NMR (400 MHZ, CHLOROFORM-d) δ=3.67 (dd, J=7.6, 10.0 Hz, 1H), 3.40-3.34 (m, 2H), 3.33-3.29 (m, 3H), 2.94-2.89 (m, 1H), 2.76-2.65 (m, 2H), 2.07-1.96 (m, 3H), 1.80-1.71 (m, 3H), 1.65-1.60 (m, 2H), 1.12-1.01 (m, 1H), 0.58-0.49 (m, 2H), 0.20 (q, J=4.8 Hz, 2H).

Step C. (R)-1-(2-(((3S,7aS)-3-((cyclopropylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of ((3S,7aS)-3-((cyclopropylmethoxy)methyl)tetrahydro-1H-pyrrolizin-7a(5H)-yl)methanol (101 mg, 1.1 equiv) and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (215 mg, 1.0 equiv) in toluene (1 mL) was added t-BuONa (2 M, 4.0 equiv). The reaction was stirred at 0° C. for 1 hour. The mixture was quenched with H₂O (5 mL) and extracted with ethyl acetate (2×10 mL). The combined organic layers were dried over Na₂SO₄, filtered and concentrated to afford the title compound (460 mg, 95% yield) as yellow solid;

Step D. (R)-1-(2-(((3S,7aS)-3-((cyclopropylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of (R)-1-(2-(((3S,7aS)-3-((cyclopropylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (450 mg, 1.0 equiv) in ACN (3 mL) was added HCl/dioxane (4 M, 38 equiv). The reaction was stirred at 25° C. for 0.5 hours. The mixture was concentrated, dissolved in MeOH (3 mL), neutralized with solid NaHCO₃ and filtered. The filtrate was concentrated and purified by prep-HPLC [column: Phenomenex luna C18 150×25 mm×10 um; mobile phase: [water (FA)-ACN]; B %: 21%-51%, 10 min] to afford the title compound (84.9 mg, 19% yield, HCOOH) as yellow solid; ¹H NMR (400 MHZ, METHANOL-d4) δ=9.27 (d, J=3.2 Hz, 1H), 7.69 (dd, J=6.0, 8.8 Hz, 1H), 7.32 (d, J=2.4 Hz, 1H), 7.26 (t, J=9.2 Hz, 1H), 7.05 (br s, 1H), 4.70-4.57 (m, 4H), 4.42-4.31 (m, 1H), 4.24-4.11 (m, 1H), 3.86-3.77 (m, 1H), 3.75-3.60 (m, 2H), 3.60-3.52 (m, 1H), 3.50-3.37 (m, 3H), 2.50-2.33 (m, 3H), 2.27-2.13 (m, 4H), 2.11-1.98 (m, 4H), 1.93-1.74 (m, 3H), 1.31 (d, J=8.4 Hz, 3H), 1.10-0.98 (m, 1H), 0.81 (td, J=7.2, 14.0 Hz, 3H), 0.49 (br d, J=7.6 Hz, 2H), 0.19 (br t, J=3.2 Hz, 2H); LCMS (ESI, M+1): m/z=674.6.

### Example 871

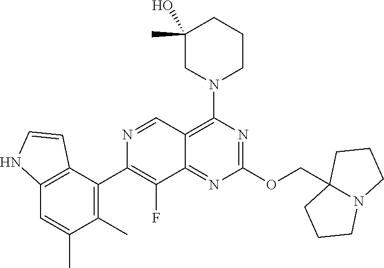 [SMILES: Cc1cc2[nH]ccc2c(-c2ncc3c(N4CCC[C@@](C)(O)C4)nc(OCC45CCCN4CCC5)nc3c2F)c1C]

### (3R)-1-(7-(5,6-dimethyl-1H-indol-4-yl)-8-fluoro-2-((tetrahydro-1H-pyrrolizin-7a(5H)-yl)methoxy)pyrido[4,3-d]pyrimidin-4-yl)-3-methylpiperidin-3-ol

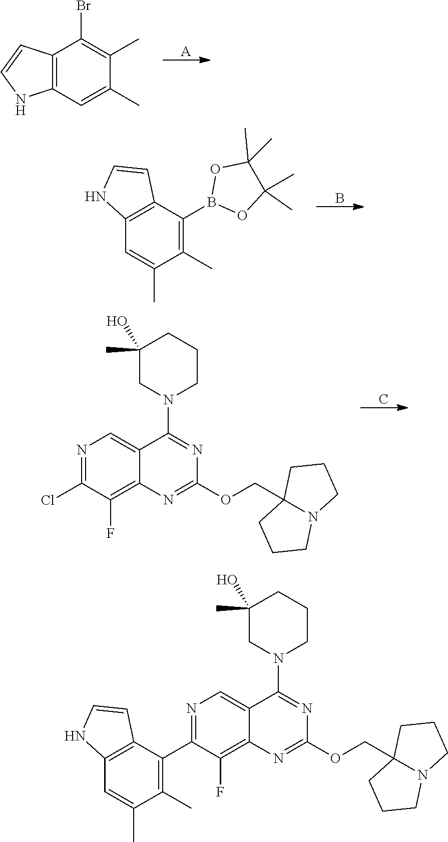

Step A. 5,6-dimethyl-4-(4,4,5,5-tetramethyl-1,3,2-dioxaborolan-2-yl)-1H-indole: To a mixture of 4-bromo-5,6-dimethyl-1H-indole (100 mg, 1.0 equiv), 4,4,5,5-tetramethyl-1,3,2-dioxaborolane (571 mg, 10 equiv) and (1,1′-bis(diphenylphosphino)ferrocene)palladium(II) dichloride (32.6 mg, 0.1 equiv) in MeCN (1.0 mL) was added TEA (135 mg, 3.0 equiv). The reaction was degassed and purged with nitrogen 3 times. The reaction was stirred at 90° C. for 12 hours. The mixture was filtered and purified by reversed phase flash chromatography [water (FA, 0.1%)/acetonitrile] to afford the title compound (96 mg, 73% yield) as yellow oil; LCMS (ESI, M+1): m/z=272.1.

Step B. (R)-1-(7-chloro-8-fluoro-2-((hexahydro-1H-pyrrolizin-7a-yl)methoxy)pyrido[4,3-d]pyrimidin-4-yl)-3-methylpiperidin-3-ol: To a solution of 7-chloro-8-fluoro-2-((hexahydro-1H-pyrrolizin-7a-yl)methoxy)-4-(2,2,2-trifluoroethoxy)pyrido[4,3-d]pyrimidine (200 mg, 1.0 equiv) and (R)-3-methylpiperidin-3-ol (109 mg, 2.0 equiv) in DMF (1.0 mL) were added DIEA (1.23 g, 20 equiv) and 4 Å molecular sieve (20.0 mg, 1.0 equiv). The reaction was stirred 60° C. for 12 hours. The mixture was filtered and purified by reversed phase flash chromatography [water (FA, 0.1%)/acetonitrile] to afford the title compound (160 mg, 62% yield) as yellow solid. LCMS (ESI, M+1): m/z=436.1.

Step C. (3R)-1-(7-(5,6-dimethyl-1H-indol-4-yl)-8-fluoro-2-((hexahydro-1H-pyrrolizin-7a-yl)methoxy)pyrido[4,3-d]pyrimidin-4-yl)-3-methylpiperidin-3-ol: To a mixture of (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (120 mg, 1.0 equiv) and 5,6-dimethyl-4-(4,4,5,5-tetramethyl-1,3,2-dioxaborolan-2-yl)-1H-indole (90.0 mg, 1.2 equiv) in H₂O (3.0 mL) and THF (10 mL) were added CataCXium A Pd G3 (20.1 mg, 0.1 equiv) and K₃PO₄ (176 mg, 3.0 equiv). The reaction was degassed and purged with nitrogen 3 times. The reaction was stirred 60° C. for 2 hours. The mixture was washed with water (2 mL) and extracted with EtOAc (2 mL×3). The combined organic layers were dried over anhydrous sodium sulfate, concentrated, and purified with HPLC (column: Phenomenex luna C18 150*25 mm*10 um; mobile phase: [water (FA)-ACN]; B %: 8%-38%, 10 min) to afford the title compound (23.1 mg, 14% yield, 0.59 HCOOH) as yellow solid. ¹H NMR (400 MHz, METHANOL-d4) δ=9.27 (d, J=5.6 Hz, 1H), 7.36 (s, 1H), 7.13 (d, J=3.2 Hz, 1H), 5.97 (dd, J=3.2, 7.6 Hz, 1H), 4.66-4.56 (m, 3H), 4.33 (br d, J=13.2 Hz, 1H), 3.64 (br dd, J=1.6, 13.6 Hz, 1H), 3.53 (td, J=5.6, 11.2 Hz, 2H), 3.48-3.40 (m, 1H), 3.13 (td, J=6.4, 11.2 Hz, 2H), 2.45 (s, 3H), 2.31-2.31 (m, 1H), 2.31-2.22 (m, 1H), 2.20-2.14 (m, 4H), 2.14-2.06 (m, 3H), 2.06-1.93 (m, 3H), 1.90-1.74 (m, 3H), 1.29 (s, 3H); LCMS (ESI, M+1): m/z=545.3.

### Example 872

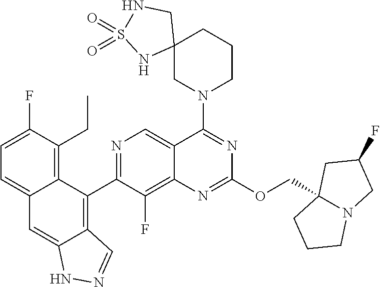 [SMILES: CCc1c(F)ccc2cc3[nH]ncc3c(-c3ncc4c(N5CCCC6(CNS(=O)(=O)N6)C5)nc(OC[C@@]56CCCN5C[C@H](F)C6)nc4c3F)c12]

### 7-(7-(5-ethyl-6-fluoro-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decane

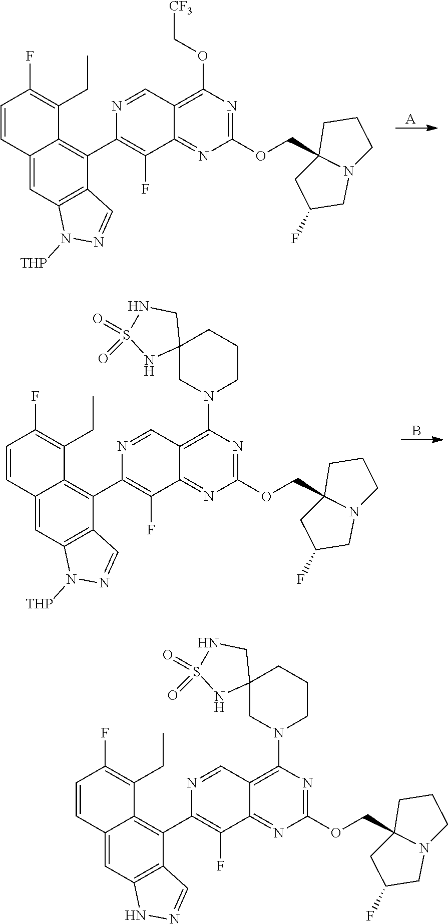

Step A. 7-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,7-triazaspiro[4.5]decane 2,2-dioxide: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole (9.00 mg, 1.0 equiv) and 2-thia-1,3,7-triazaspiro[4.5]decane 2,2-dioxide (4.91 mg, 2.0 equiv) in DMF (0.2 mL) were added DIEA (16.6 mg, 10 equiv) and 4 Å molecular sieve (1.00 mg, 1.0 equiv). The mixture was stirred at 60° C. for 12 hours. The mixture was filtered and purified by reversed phase flash chromatography [water (FA, 0.1%)/acetonitrile] to afford the title compound (6.00 mg, 52% yield) as yellow solid. LCMS (ESI, M+1): m/z=792.5.

Step B. 7-(7-(5-ethyl-6-fluoro-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,7-triazaspiro[4.5]decane 2,2-dioxide: A mixture of 7-(7-(5-ethyl-6-fluoro-1-(tetrahydro-2H-pyran-2-yl)-1H-benzo[f]indazol-4-yl)-8-fluoro-2-(((2R,7aS)-2-fluorohexahydro-1H-pyrrolizin-7a-yl)methoxy)pyrido[4,3-d]pyrimidin-4-yl)-2-thia-1,3,7-triazaspiro[4.5]decane 2,2-dioxide (12.0 mg, 1.0 equiv) in TFA (308 mg, 0.2 mL, 178 equiv) was stirred at 25° C. for 0.5 hour. The mixture was concentrated. The residue was diluted with water (1 mL). The mixture was adjusted to pH=7 with saturated NaHCO₃ (10 mL) and extracted with EtOAc (2 mL×3). The combined organic layers were dried over anhydrous sodium sulfate, concentrated, and purified with prep-HPLC (column: Phenomenex luna C18 150*25 mm*10 um; mobile phase: [water (FA)-ACN]; B %: 15%-45%, 10 min) to afford the title compound (2.09 mg, 18% yield, 0.34 HCOOH) as yellow solid. ¹H NMR (400 MHZ, METHANOL-d4) δ=9.21 (d, J=7.2 Hz, 1H), 8.24 (d, J=3.2 Hz, 1H), 8.01 (br dd, J=5.6, 8.8 Hz, 1H), 7.87-7.62 (m, 1H), 7.33 (t, J=9.6 Hz, 1H), 5.41-5.33 (m, 1H), 4.41 (br t, J=11.2 Hz, 2H), 3.95-3.78 (m, 2H), 3.75-3.57 (m, 2H), 3.46-3.38 (m, 2H), 3.26-3.06 (m, 3H), 2.72-2.60 (m, 1H), 2.52-2.17 (m, 5H), 2.15-1.78 (m, 7H), 0.95-0.78 (m, 3H); LCMS (ESI, M+1): m/z=708.6.

### Example 873

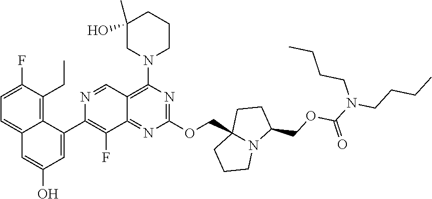 [SMILES: CCCCN(CCCC)C(=O)OC[C@@H]1CC[C@@]2(COc3nc(N4CCC[C@@](C)(O)C4)c4cnc(-c5cc(O)cc6ccc(F)c(CC)c56)c(F)c4n3)CCCN12]

### ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl dibutylcarbamate

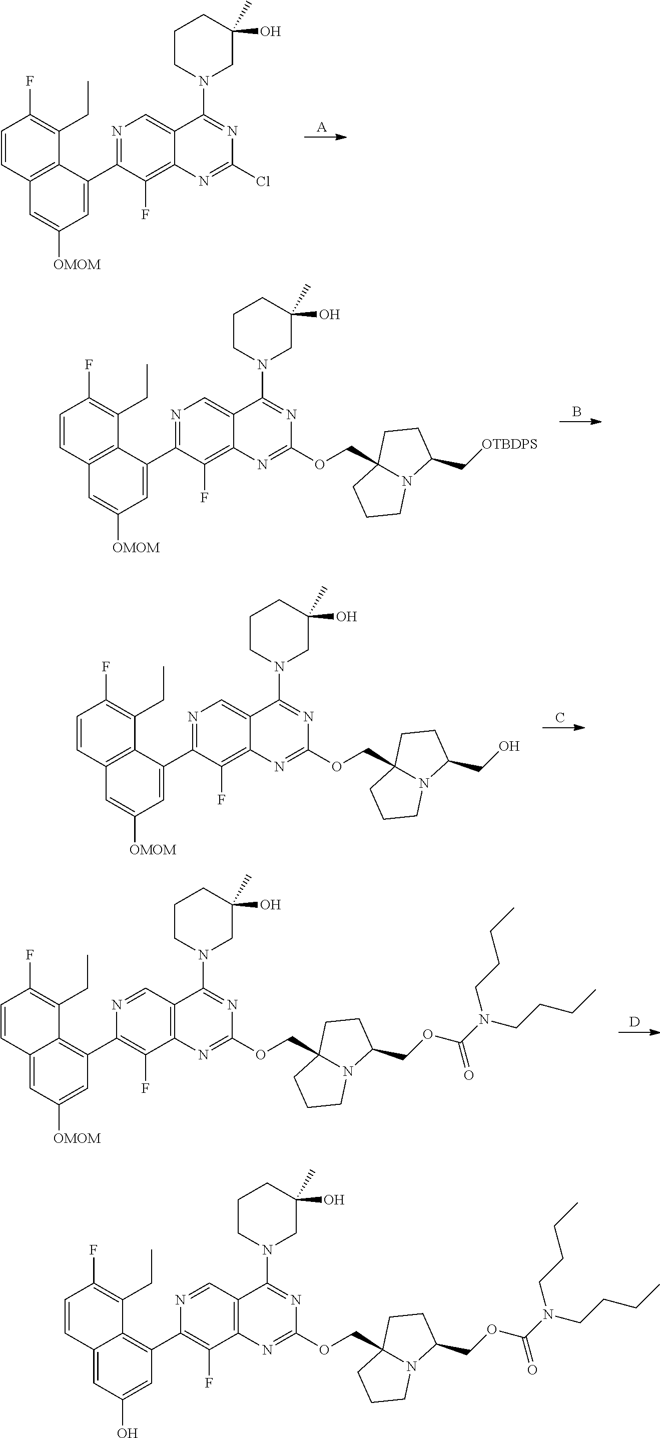

Step A. (R)-1-(2-(((3S,7aR)-3-(((tert-butyldiphenylsilyl)oxy)methyl) hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of ((3S,7aR)-3-(((tert-butyldiphenylsilyl)oxy)methyl) hexahydro-1H-pyrrolizin-7a-yl)methanol (302 mg, 1.3 equiv) and 4 Å molecular sieve (10.0 mg, 1.0 equiv) in DMF (1 mL) was added t-BuONa (2 M, 2.0 equiv). The reaction mixture was stirred at 0° C. for 40 minutes. (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (300 mg, 1.0 equiv) was added into the mixture. The reaction was stirred at 0° C. for 40 minutes. The mixture was diluted with water (10 mL) and extracted with EtOAc (3×30 mL). The combined organic layers were dried over anhydrous sodium sulfate, concentrated and purified with prep-TLC [SiO₂, DCM/MeOH=10/1] to afford the title compound (400 mg, 70% yield) as yellow solid. LCMS (ESI, M+1): m/z=902.3

Step B. (R)-1-(7-(8-ethyl-7-fluoro-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3-methylpiperidin-3-ol: To a solution of (R)-1-(2-(((3S,7aR)-3-(((tert-butyldiphenylsilyl)oxy)methyl) hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (400 mg, 1.0 equiv) in DMF (5 mL) was added CsF (1.01 g, 15 equiv). The reaction was stirred at 25° C. for 24 hours. The mixture was diluted with EtOAc (20 mL), washed with brine (20 mL×3). The organic layer was dried over anhydrous sodium sulfate, concentrated and purified with prep-TLC [SiO₂, DCM/MeOH=10/1] to afford the title compound (180 mg, 61% yield) as off-red solid. LCMS (ESI, M+1): m/z=664.3

Step C. ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl dibutylcarbamate: To a mixture of (R)-1-(7-(8-ethyl-7-fluoro-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3-methylpiperidin-3-ol (90.0 mg, 1.0 equiv) in THF (1.5 mL) was added TEA (137 mg, 0.2 mL, 10 equiv). The reaction was stirred at 25° C. for 4 minutes. 4-nitrophenyl carbonochloridate in THF (0.5 mL) was added into the mixture at 0° C. The reaction was stirred at 40° C. for 5 hours. TEA (137 mg, 0.2 mL, 10 equiv) and N-butylbutan-1-amine (52.6 mg, 0.7 mL, 3.0 equiv) were added into above mixture. The reaction was stirred at 80° C. for 12 hours. The mixture was diluted with water (10 mL) and extracted with ethyl acetate (2×10 ml). The organic layer was dried over anhydrous sodium sulfate, concentrated and purified with prep-TLC [SiO₂, DCM/MeOH=10/1] to afford the title compound (30 mg, 27% yield) as yellow oil; LCMS (ESI, M+1): m/z=819.3.

Step D. ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl dibutylcarbamate: To a solution of ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl dibutylcarbamate (30 mg, 1.0 equiv) in MeCN (1 mL) was added HCl·dioxane (4 M, 1.0 mL). The reaction was stirred at 0° C. for 30 minutes. The mixture was adjusted pH-7 with NaHCO₃ solution. The mixture was filtered and purified with prep-HPLC (column: Waters Xbridge 150*25 mm*5 um; mobile phase: [water (NH₄HCO₃)-ACN]; B %: 80%-100%, 8 min) to afford the title compound (12.5 mg, 44% yield) as yellow solid; ¹HNMR (400 MHZ, METHANOL-d4) δ=9.22 (d, J=2.4 Hz, 1H), 7.69 (dd, J=6.0, 9.2 Hz, 1H), 7.32 (d, J=2.8 Hz, 1H), 7.27 (t, J=9.6 Hz, 1H), 7.08 (t, J=2.4 Hz, 1H), 4.61 (br s, 1H), 4.55 (br d, J=13.2 Hz, 1H), 4.37-4.21 (m, 3H), 4.10-4.04 (m, 1H), 4.03-3.96 (m, 1H), 3.71-3.57 (m, 1H), 3.54-3.43 (m, 1H), 3.26 (br s, 4H), 3.11-3.02 (m, 2H), 2.88-2.78 (m, 1H), 2.58-2.41 (m, 1H), 2.30-2.12 (m, 3H), 2.11-2.01 (m, 1H), 2.00-1.86 (m, 4H), 1.85-1.75 (m, 4H), 1.74-1.65 (m, 1H), 1.60-1.49 (m, 4H), 1.36-1.27 (m, 7H), 0.94 (t, J=7.2 Hz, 6H), 0.87-0.78 (m, 3H); LCMS (ESI, M+1): m/z=775.3.

### Example 874

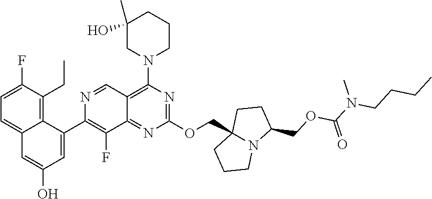 [SMILES: CCCCN(C)C(=O)OC[C@@H]1CC[C@@]2(COc3nc(N4CCC[C@@](C)(O)C4)c4cnc(-c5cc(O)cc6ccc(F)c(CC)c56)c(F)c4n3)CCCN12]

### ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl butyl(methyl)carbamate

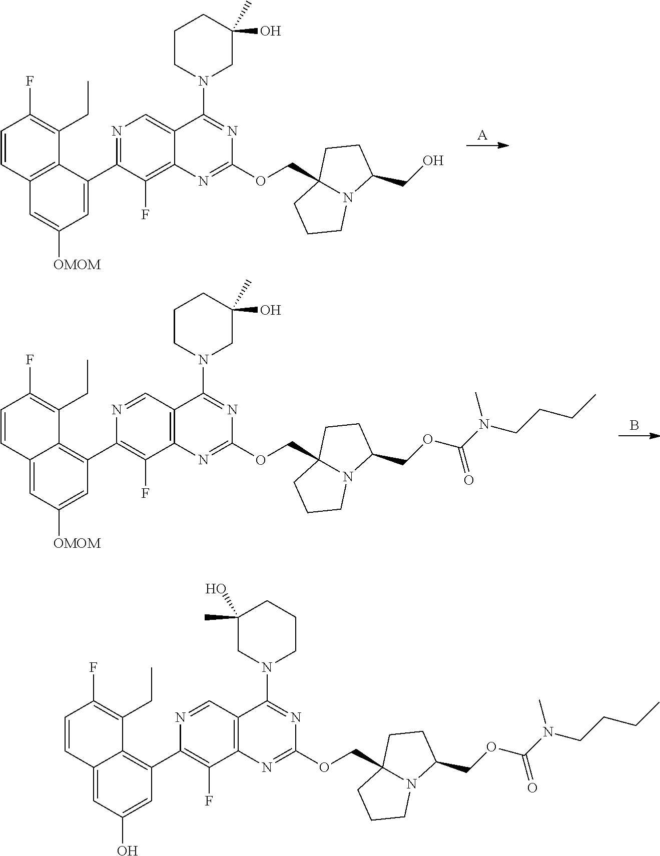

Step A. ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl butyl(methyl)carbamate: To a mixture of (R)-1-(7-(8-ethyl-7-fluoro-3-(methoxymethoxy)naphthalen-1-yl)-8-fluoro-2-(((3S,7aR)-3-(hydroxymethyl) hexahydro-1H-pyrrolizin-7a-yl)methoxy)pyrido[4,3-d]pyrimidin-4-yl)-3-methylpiperidin-3-ol (90.0 mg, 1.0 equiv) in THF (1.5 mL) was added TEA (137 mg, 0.2 mL, 10 equiv). The reaction was stirred at 25° C. for 4 minutes. 4-nitrophenyl carbonochloridate in THF (0.5 mL) was added into the mixture at 0° C. The reaction was stirred at 40° C. for 5 hours. TEA (137 mg, 0.2 mL, 10 equiv) and N-methylbutan-1-amine (35.5 mg, 3.0 equiv) were added into above mixture. The reaction was stirred at 80° C. for 12 hours. The mixture was diluted with water (10 mL) and extracted with ethyl acetate (2×10 ml). The combined organic layers were dried over anhydrous sodium sulfate, concentrated and purified with prep-TLC [SiO₂, DCM/MeOH=10/1] to afford the title compound (30.0 mg, 28% yield) as yellow oil; LCMS (ESI, M+1): m/z=777.3.

Step B. ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl butyl(methyl)carbamate: To a solution of ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl butyl(methyl)carbamate (30.0 mg, 1.0 equiv) in MeCN (1 mL) was added HCl·dioxane (4M, 1 mL). The reaction mixture was stirred at 0° C. for 30 minutes. The mixture was adjusted pH=7 with NaHCO₃ solution. The mixture was filtered and purified with prep-HPLC (column: Phenomenex C18 75*30 mm*3 um; mobile phase: [water (FA)-ACN]; B %: 2%-32%, 7 min) to afford the title compound (20 mg, 71% yield) as white solid; ¹H NMR (400 MHz, METHANOL-d4) δ=9.24 (d, J=2.4 Hz, 1H), 7.70 (dd, J=5.6, 9.2 Hz, 1H), 7.32 (d, J=2.8 Hz, 1H), 7.27 (t, J=9.2 Hz, 1H), 7.08 (d, J=2.4 Hz, 1H), 4.57 (br d, J=15.6 Hz, 2H), 4.44-4.27 (m, 3H), 4.25-4.12 (m, 1H), 4.07 (br dd, J=6.4, 10.8 Hz, 1H), 3.73-3.57 (m, 1H), 3.54-3.45 (m, 1H), 3.26 (br d, J=6.4 Hz, 3H), 3.08-2.97 (m, 1H), 2.95-2.84 (m, 3H), 2.48 (br dd, J=7.2, 14.8 Hz, 1H), 2.36-2.09 (m, 4H), 2.07-1.96 (m, 3H), 1.95-1.73 (m, 6H), 1.51 (br s, 2H), 1.37-1.22 (m, 5H), 0.93 (t, J=7.2 Hz, 3H), 0.87-0.78 (m, 3H); LCMS (ESI, M+1): m/z=733.2.

### Example 875

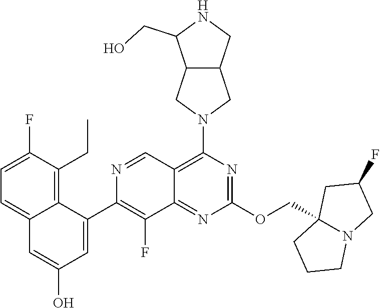 [SMILES: CCc1c(F)ccc2cc(O)cc(-c3ncc4c(N5CC6CNC(CO)C6C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(4-(hydroxymethyl)hexahydropyrrolo[3,4-c]pyrrol-2 (1H)-yl)pyrido[4,3-d]pyrimidin-7-yl)naphthalen-2-ol

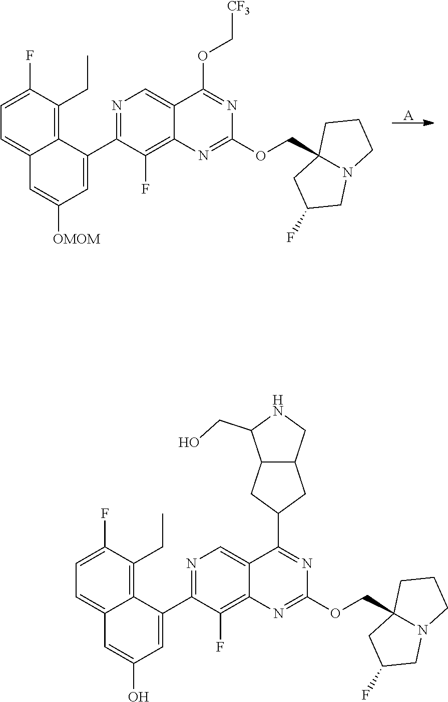

Step A. 5-ethyl-6-fluoro-4-(8-fluoro-2-(((2R,7aS)-2-fluorohexahydro-1H-pyrrolizin-7a-yl)methoxy)-4-(4-(hydroxymethyl)hexahydropyrrolo[3,4-c]pyrrol-2 (1H)-yl)pyrido[4,3-d]pyrimidin-7-yl)naphthalen-2-ol: To a mixture of 7-(8-ethyl-7-fluoro-3-(methoxymethoxy)naphthalen-1-yl)-8-fluoro-2-(((2R,7aS)-2-fluorohexahydro-1H-pyrrolizin-7a-yl)methoxy)-4-(2,2,2-trifluoroethoxy)pyrido[4,3-d]pyrimidine (59.2 mg, 1.0 equiv) and (octahydropyrrolo[3,4-c]pyrrol-1-yl)methanol (20.0 mg, 1.0 equiv, 2 HCl salt) in DMF (0.5 mL) were added DIEA (60.1 mg, 5.0 equiv) and 4 Å molecular sieve (50 mg). The reaction was stirred at 40° C. for 12 hours. The mixture was filtered and purified by prep-HPLC (column: Phenomenex luna C18 150*25 mm*10 um; mobile phase: [water (FA)-ACN]; B %: 3%-33%, 10 min) to afford the title compound (15.0 mg, 25% yield) as a yellow solid. ¹H NMR (400 MHZ, METHANOL-d₄) δ=9.28 (s, 1H), 8.49 (br s, 2H), 7.68 (br dd, J=6.0, 8.8 Hz, 1H), 7.32 (br d, J=2.0 Hz, 1H), 7.25 (br t, J=9.2 Hz, 1H), 7.05 (br d, J=2.0 Hz, 1H), 5.59-5.34 (m, 1H), 4.54 (br s, 2H), 4.33 (br s, 2H), 4.27-4.08 (m, 2H), 3.97-3.54 (m, 7H), 3.44-3.33 (m, 2H), 3.27 (br s, 1H), 3.12 (br s, 1H), 2.68-2.37 (m, 3H), 2.35-2.05 (m, 5H), 0.89-0.62 (m, 3H); LCMS (ESI, M+1): m/z=635.1.

### Example 876

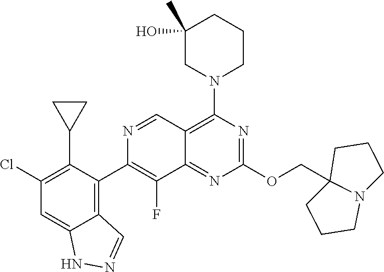 [SMILES: C[C@@]1(O)CCCN(c2nc(OCC34CCCN3CCC4)nc3c(F)c(-c4c(C5CC5)c(Cl)cc5[nH]ncc45)ncc23)C1]

### (3R)-1-(7-(6-chloro-5-cyclopropyl-1H-indazol-4-yl)-8-fluoro-2-((tetrahydro-1H-pyrrolizin-7a(5H)-yl)methoxy)pyrido[4,3-d]pyrimidin-4-yl)-3-methylpiperidin-3-ol

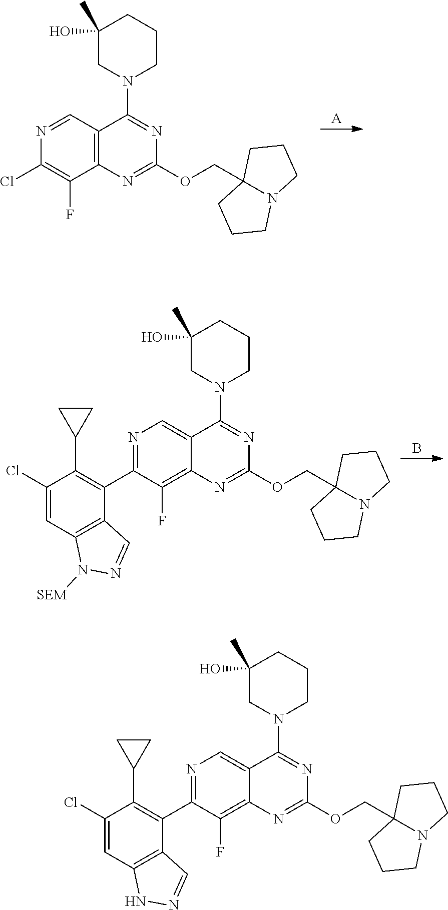

Step A. (3R)-1-(7-(6-chloro-5-cyclopropyl-1-((2-(trimethylsilyl) ethoxy)methyl)-1H-indazol-4-yl)-8-fluoro-2-((hexahydro-1H-pyrrolizin-7a-yl)methoxy)pyrido[4,3-d]pyrimidin-4-yl)-3-methylpiperidin-3-ol: To a solution of (R)-1-(7-chloro-8-fluoro-2-((hexahydro-1H-pyrrolizin-7a-yl)methoxy)pyrido[4,3-d]pyrimidin-4-yl)-3-methylpiperidin-3-ol (200 mg, 1.0 equiv) and 6-chloro-5-cyclopropyl-4-(4,4,5,5-tetramethyl-1,3,2-dioxaborolan-2-yl)-1-((2-(trimethylsilyl) ethoxy)methyl)-1H-indazole (268 mg, 1.3 equiv) in toluene (5 mL) were added K₃PO₄ (1.5 M, 917 μL, 3.0 equiv) and APhos Pd G3 (29.1 mg, 0.1 equiv). The reaction was degassed and purged with nitrogen 3 times. The mixture was stirred at 60° C. for 12 hours. The mixture was diluted with H₂O (20 mL) and extracted with EtOAc (3×20 mL). The combined organic layers were dried over anhydrous sodium sulfate, concentrated and purified by column chromatography with prep-TLC [SiO₂, DCM:MeOH=10:1] to afford the title compound (150 mg, 38% yield) as a yellow solid; LCMS (ESI, M+1): m/z=722.4.

Step B. (3R)-1-(7-(6-chloro-5-cyclopropyl-1H-indazol-4-yl)-8-fluoro-2-((hexahydro-1H-pyrrolizin-7a-yl)methoxy)pyrido[4,3-d]pyrimidin-4-yl)-3-methylpiperidin-3-ol: To a solution of (3R)-1-(7-(6-chloro-5-cyclopropyl-1-((2-(trimethylsilyl) ethoxy)methyl)-1H-indazol-4-yl)-8-fluoro-2-((hexahydro-1H-pyrrolizin-7a-yl)methoxy)pyrido[4,3-d]pyrimidin-4-yl)-3-methylpiperidin-3-ol (112 mg, 1.0 equiv) in DCM (0.5 mL) was added TFA (1.54 g, 1.0 mL, 97 equiv) at 0° C. The reaction was stirred at 25° C. for 5 hours. The mixture was concentrated. The residue was diluted with water (1 mL). The mixture was adjusted to pH=7 with saturated NaHCO₃ (5 mL) and extracted with DCM (3×10 mL). The combined organic layers were dried over anhydrous sodium sulfate, concentrated and purified with prep-HPLC [Phenomenex Synergi C18 150×25 mm×10 μm; A: water (NH₄HCO₃), B: ACN; B %: 25%-55% over 8 min] to afford the title compound (36.9 mg, 43% yield) as a light yellow solid; ¹H NMR (400 MHZ, METHANOL-d₄) δ=9.31 (d, J=5.8 Hz, 1H), 7.83-7.79 (m, 1H), 7.77 (s, 1H), 4.59-4.49 (m, 1H), 4.39-4.23 (m, 3H), 3.70-3.57 (m, 1H), 3.53-3.42 (m, 1H), 3.16-3.04 (m, 2H), 2.77-2.65 (m, 2H), 2.24-2.00 (m, 4H), 1.99-1.70 (m, 9H), 1.31 (d, J=8.6 Hz, 3H), 0.96-0.81 (m, 1H), 0.71-0.57 (m, 1H), 0.33-0.20 (m, 1H), 0.14-0.02 (m, 1H); LCMS (ESI, M+1): m/z=592.3.

### Example 877

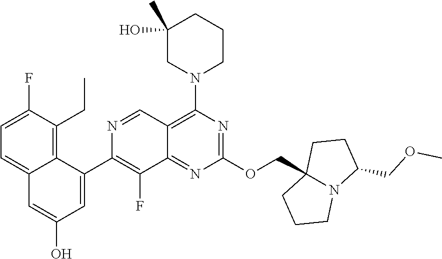 [SMILES: CCc1c(F)ccc2cc(O)cc(-c3ncc4c(N5CCC[C@@](C)(O)C5)nc(OC[C@]56CCCN5[C@@H](COC)CC6)nc4c3F)c12]

### (R)-1-(7-(8-ethyl-7-fluoro-3-hydroxynaphthalen-1-yl)-8-fluoro-2-(((3R,7aR)-3-(methoxymethyl)tetrahydro-1H-pyrrolizin-7a(5H)-yl)methoxy)pyrido[4,3-d]pyrimidin-4-yl)-3-methylpiperidin-3-ol

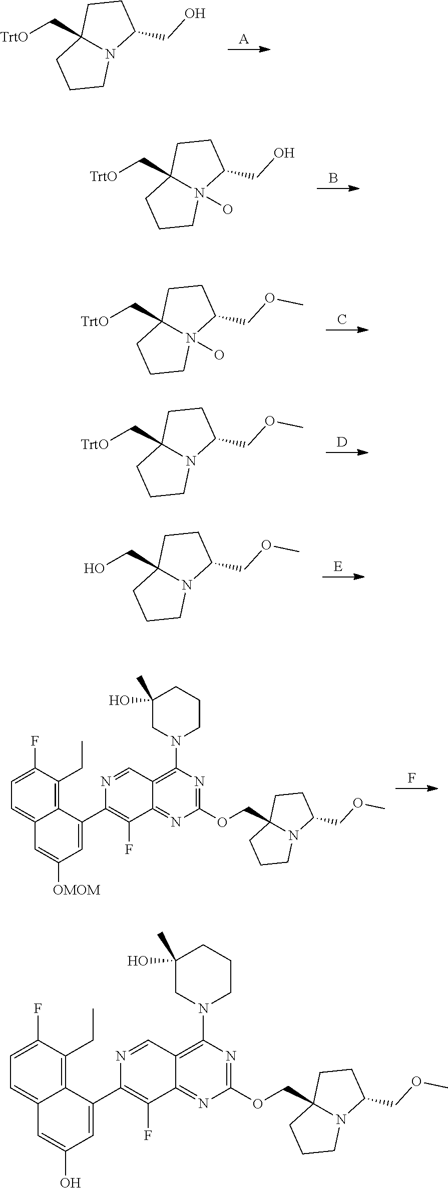

Step A. (3R,7aR)-3-(hydroxymethyl)-7a-((trityloxy)methyl) octahydropyrrolizine 4-oxide: To a solution of ((3R,7aR)-7a-((trityloxy)methyl) hexahydro-1H-pyrrolizin-3-yl)methanol (500 mg, 1.0 equiv) in DCM (6 mL) was added m-CPBA (295 mg, 85% purity, 1.2 equiv). The reaction was stirred at 25° C. for 2 hours. The mixture was quenched by addition of saturated NaHCO₃ aqueous solution (0.5 mL) and extracted with DCM (10 mL). The organic layer was washed with saturated NaHCO₃ aqueous solution (10 mL) and brine (10 mL), dried over sodium sulfate and concentrated to afford the title compound (520 mg, crude) as a white solid; LCMS (ESI, M+1): m/z=430.1.

Step B. (3R,7aR)-3-(methoxymethyl)-7a-((trityloxy)methyl) octahydropyrrolizine 4-oxide: To a solution of (3R,7aR)-3-(hydroxymethyl)-7a-((trityloxy)methyl) octahydropyrrolizine 4-oxide (520 mg, 1.0 equiv) in DMF (5 mL) was added NaH (72.6 mg, 60% purity, 1.5 equiv). The mixture was stirred at 0° C. for 30 minutes. MeI (258 mg, 1.5 equiv) was added. The reaction was stirred at 0° C. for 2 hours. The mixture was quenched by addition of ice-water (15 mL) and extracted with ethyl acetate (2×15 mL). The combined organic layers were washed with brine (15 mL), dried over sodium sulfate, concentrated and purified by prep-HPLC [column: Waters Xbridge 150×25 mm×5 μm; A: water (NH₃·H₂O), B: ACN, B %: 45%-75% over 9 min] to afford the title compound (80.0 mg, 15% yield) as a yellow solid; LCMS (ESI, M+1): m/z=444.2.

Step C. (3R,7aR)-3-(methoxymethyl)-7a-((trityloxy)methyl) hexahydro-1H-pyrrolizine: To a solution of (3R,7aR)-3-(methoxymethyl)-7a-((trityloxy)methyl) octahydropyrrolizine 4-oxide (80.0 mg, 1.0 equiv) in MeOH (1 mL) was added Pd/C (10.0 mg, 10% purity) under N₂ atmosphere. The reaction was degassed and purged with H₂ for 3 times and stirred at 25° C. for 12 hours under H₂ (15 Psi) atmosphere. The mixture was filtered through Celite and the filter cake was washed with MeOH (10 mL). The filtrate was concentrated to afford the title compound (75.0 mg, crude) as a yellow solid; LCMS (ESI, M+1): m/z=428.3.

Step D. ((3R,7aR)-3-(methoxymethyl) hexahydro-1H-pyrrolizin-7a-yl)methanol: To a solution of (3R,7aR)-3-(methoxymethyl)-7a-((trityloxy)methyl) hexahydro-1H-pyrrolizine (75.0 mg, 1.0 equiv) in DCM (0.6 mL) was added TFA (0.3 mL), and then the mixture was stirred at 20° C. for 2 hours. The mixture was concentrated under reduced pressure to afford the title compound (75.0 mg, crude) as a yellow solid.

Step E. (R)-1-(7-(8-ethyl-7-fluoro-3-(methoxymethoxy)naphthalen-1-yl)-8-fluoro-2-(((3R,7aR)-3-(methoxymethyl) hexahydro-1H-pyrrolizin-7a-yl)methoxy)pyrido[4,3-d]pyrimidin-4-yl)-3-methylpiperidin-3-ol: A mixture of ((3R,7aR)-3-(methoxymethyl) hexahydro-1H-pyrrolizin-7a-yl)methanol (38.5 mg, 1.1 equiv), 4 Å molecular sieve (50 mg) and t-BuONa (72.7 mg, 4.0 equiv) in toluene (1 mL) was stirred at 0° C. for 10 minutes. (S)-2-chloro-7-(8-ethyl-7-fluoro-3-(methoxymethoxy)naphthalen-1-yl)-8-fluoro-4-(3-methylpiperidin-1-yl)pyrido[4,3-d]pyrimidine (100 mg, 1.0 equiv) was added. The reaction was stirred at 0° C. for additional 1 hour. The mixture was quenched with H₂O (10 mL) at 0° C. and extracted with ethyl acetate (3×10 mL). The combined organic layers were washed with brine (20 mL), dried over sodium sulfate, concentrated and purified by prep-HPLC [column: Phenomenex Luna C18 75×30 mm×3 μm; A: water (FA), B: ACN, B %: 25%-45% over 8 min] to afford the title compound (25.0 mg, 19% yield) as a white solid; LCMS (ESI, M+1): m/z=678.4.

Step F. (R)-1-(7-(8-ethyl-7-fluoro-3-hydroxynaphthalen-1-yl)-8-fluoro-2-(((3R,7aR)-3-(methoxymethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethyl-7-fluoro-3-(methoxymethoxy)naphthalen-1-yl)-8-fluoro-2-(((3R,7aR)-3-(methoxymethyl) hexahydro-1H-pyrrolizin-7a-yl)methoxy)pyrido[4,3-d]pyrimidin-4-yl)-3-methylpiperidin-3-ol (20.0 mg, 1.0 equiv) in MeOH (200 uL) was added HCl/MeOH (4 M, 200 uL) at 0° C. The mixture was stirred at 25° C. for 0.5 hour. The mixture was concentrated, diluted with MeCN (0.5 mL) and adjusted to pH=˜ 7 with 10% NH₃·H₂O. The mixture was directly purified by prep-HPLC [column: Phenomenex Luna C18 75×30 mm×3 μm; A: water (FA), B: ACN, B %: 18%-48% over 7 min] to afford the title compound (15.0 mg, 78% yield) as a yellow solid. ¹H NMR (400 MHZ, METHANOL-d₄) δ=9.29-9.15 (m, 1H), 8.55 (s, 1H), 7.72-7.61 (m, 1H), 7.33-7.29 (m, 1H), 7.25 (br t, J=9.2 Hz, 1H), 7.06 (s, 1H), 4.66-4.43 (m, 2H), 4.39-4.23 (m, 2H), 3.70-3.50 (m, 2H), 3.49-3.41 (m, 2H), 3.38 (d, J=3.6 Hz, 1H), 3.34 (d, J=2.8 Hz, 2H), 3.17-2.90 (m, 2H), 2.52-2.40 (m, 1H), 2.36-1.67 (m, 14H), 1.32-1.25 (m, 3H), 0.87-0.75 (m, 3H); LCMS (ESI, M+1): m/z=634.4.

### Example 878

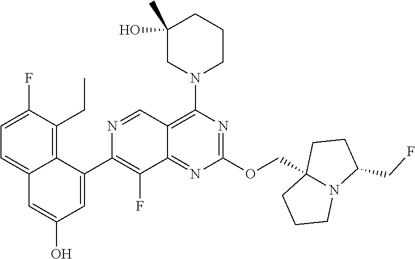 [SMILES: CCc1c(F)ccc2cc(O)cc(-c3ncc4c(N5CCC[C@@](C)(O)C5)nc(OC[C@@]56CCCN5[C@@H](CF)CC6)nc4c3F)c12]

### (R)-1-(7-(8-ethyl-7-fluoro-3-hydroxynaphthalen-1-yl)-8-fluoro-2-(((3R,7aS)-3-(fluoromethyl)tetrahydro-1H-pyrrolizin-7a(5H)-yl)methoxy)pyrido[4,3-d]pyrimidin-4-yl)-3-methylpiperidin-3-ol

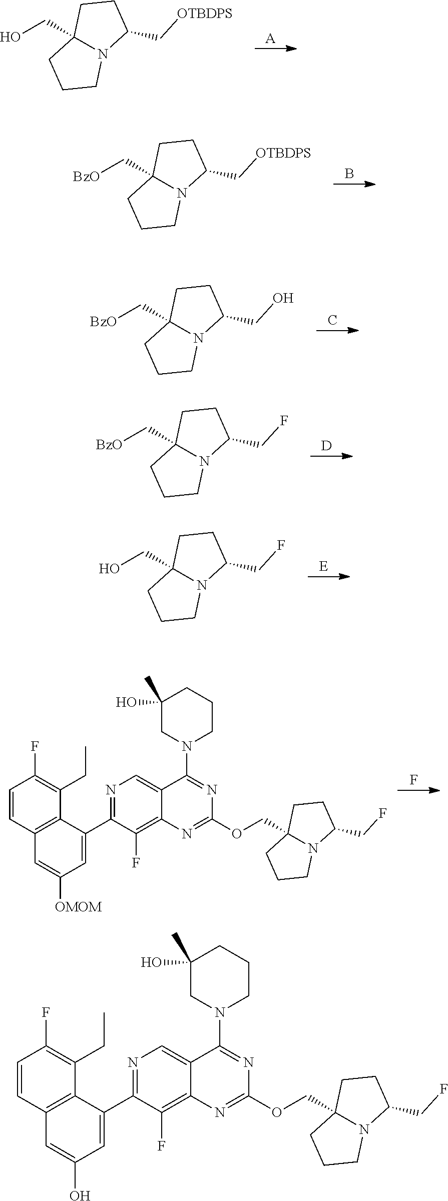

Step A. ((3R,7aS)-3-(((tert-butyldiphenylsilyl)oxy)methyl) hexahydro-1H-pyrrolizin-7a-yl)methyl benzoate: To a solution of ((3R,7aS)-3-(((tert-butyldiphenylsilyl)oxy)methyl) hexahydro-1H-pyrrolizin-7a-yl)methanol (2.60 g, 1.0 equiv) and TEA (1.28 g, 2.0 equiv) in DCM (26 mL) was added B₂Cl (1.34 g, 1.5 equiv) at 0° C. The reaction was stirred at 25° C. for 12 hours. The mixture was diluted with DCM (100 mL), dried over sodium sulfate, concentrated and purified by flash silica gel chromatography (ISCO®; 40 g SepaFlash® Silica Flash Column, Eluent of 0˜30% Ethyl acetate/Petroleum ethergradient@80 mL/min) to afford the title compound (2.30 g, 71% yield) as a yellow oil.

Step B. ((3R,7aS)-3-(hydroxymethyl) hexahydro-1H-pyrrolizin-7a-yl)methyl benzoate: To a solution of ((3R,7aS)-3-(((tert-butyldiphenylsilyl)oxy)methyl) hexahydro-1H-pyrrolizin-7a-yl)methyl benzoate (8.00 g, 1.0 equiv) in DMF (100 mL) was added CsF (35.5 g, 15.0 equiv). The reaction was stirred at 40° C. for 12 hours. The mixture was concentrated and purified by reversed phase flash chromatography [C18, 0.1% formic acid condition] to afford the title compound (2.60 g, 61% yield) as a yellow oil; LCMS (ESI, M+1): m/z=276.1.

Step C. ((3R,7aS)-3-(fluoromethyl) hexahydro-1H-pyrrolizin-7a-yl)methyl benzoate: To a solution of ((3R,7aS)-3-(hydroxymethyl) hexahydro-1H-pyrrolizin-7a-yl)methyl benzoate (500 mg, 1.0 equiv) in DCM (15 mL) was added MOST (954 mg, 3.0 equiv) at 0° C. carefully. The reaction was stirred at 20° C. for 72 hours. The mixture was diluted with ice-cold sat. NaHCO₃ aqueous solution (100 mL) and extracted with DCM (2×50 mL). The combined organic layers were dried over sodium sulfate, concentrated and purified by SFC separation [column: DAICEL CHIRALPAK AD, 250×30 mm, 10 μm; A: CO₂, B: IPA (0.1% NH3H2O), B %: 20% B over 5.4 min] to afford the title compound (75.0 mg, 27% yield) as a yellow oil. ¹H NMR (400 MHZ, CHLOROFORM-d) δ=8.05 (d, J=8.0 Hz, 2H), 7.61-7.52 (m, 1H), 7.49-7.40 (m, 2H), 4.40 (d, J=5.6 Hz, 1H), 4.28 (d, J=5.6 Hz, 1H), 4.09 (s, 2H), 3.14-2.92 (m, 2H), 2.78 (td, J=5.6, 10.8 Hz, 1H), 2.13 (br dd, J=6.4, 11.6 Hz, 1H), 2.07-1.97 (m, 1H), 1.92-1.83 (m, 4H), 1.78-1.58 (m, 5H); LCMS (ESI, M+1): m/z=278.1.

Step D. ((3R,7aS)-3-(fluoromethyl) hexahydro-1H-pyrrolizin-7a-yl)methanol: To a solution of ((3R,7aS)-3-(fluoromethyl) hexahydro-1H-pyrrolizin-7a-yl)methyl benzoate (70.0 mg, 1.0 equiv) in MeOH (1 mL) was added NaOMe methanol solution (5 M, 101 μL, 2.0 equiv). The reaction was stirred at 0° C. for 0.5 hour. The mixture was quenched with water (1 mL) and concentrated to afford the title compound (40.0 mg, crude) as a yellow oil.

Step E. (R)-1-(7-(8-ethyl-7-fluoro-3-(methoxymethoxy)naphthalen-1-yl)-8-fluoro-2-(((3R,7aS)-3-(fluoromethyl) hexahydro-1H-pyrrolizin-7a-yl)methoxy)pyrido[4,3-d]pyrimidin-4-yl)-3-methylpiperidin-3-ol: To a solution of ((3R,7aS)-3-(fluoromethyl) hexahydro-1H-pyrrolizin-7a-yl)methanol (40.0 mg, 1.0 equiv) and (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (73.3 mg, 0.6 equiv) in toluene (1 mL) was added t-BuONa (44.4 mg, 2.0 equiv) at 0° C. The reaction was stirred at 0° C. for 1 hour. The mixture was concentrated and purified by prep-HPLC [column: Phenomenex luna C18 75×30 mm×3 μm; A: water (FA), B: ACN, B %: 20%-50% over 7 min] to afford the title compound (27.0 mg, 18% yield) as a yellow solid; LCMS (ESI, M+1): m/z=666.2.

Step F. (R)-1-(7-(8-ethyl-7-fluoro-3-hydroxynaphthalen-1-yl)-8-fluoro-2-(((3R,7aS)-3-(fluoromethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethyl-7-fluoro-3-(methoxymethoxy)naphthalen-1-yl)-8-fluoro-2-(((3R,7aS)-3-(fluoromethyl) hexahydro-1H-pyrrolizin-7a-yl)methoxy)pyrido[4,3-d]pyrimidin-4-yl)-3-methylpiperidin-3-ol (27.0 mg, 1.0 equiv) in MeOH (0.2 mL) was added HCl/MeOH (2 M, 180 μL, 8.9 equiv). The reaction was stirred at 0° C. for 2 hours. The mixture was concentrated and purified by prep-HPLC [column: Phenomenex luna C18 75×30 mm×3 μm; A: water (FA), B: ACN, B %: 15%-45% over 7 min] to afford the title compound (8.39 mg, 32% yield) as a white solid. ¹H NMR (400 MHZ, METHANOL-d₄) δ=9.23 (br s, 1H), 7.68 (dd, J=6.0, 8.8 Hz, 1H), 7.31 (d, J=2.8 Hz, 1H), 7.25 (t, J=9.2 Hz, 1H), 7.06 (s, 1H), 4.67-4.52 (m, 3H), 4.46-4.38 (m, 2H), 4.31 (br t, J=12.8 Hz, 1H), 3.72-3.55 (m, 1H), 3.51-3.36 (m, 2H), 3.29-3.18 (m, 1H), 3.17-3.02 (m, 1H), 2.56-2.40 (m, 1H), 2.38-2.24 (m, 1H), 2.23-2.13 (m, 2H), 2.13-1.97 (m, 4H), 1.97-1.83 (m, 4H), 1.82-1.73 (m, 2H), 1.29 (d, J=10.0 Hz, 3H), 0.81 (q, J=7.6 Hz, 3H); LCMS (ESI, M+1): m/z=622.4.

### Example 879

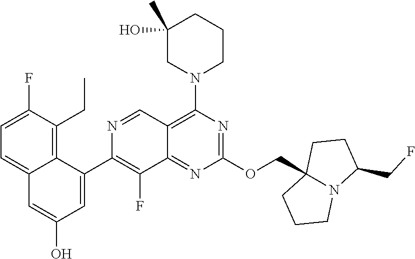 [SMILES: CCc1c(F)ccc2cc(O)cc(-c3ncc4c(N5CCC[C@@](C)(O)C5)nc(OC[C@]56CCCN5[C@H](CF)CC6)nc4c3F)c12]

### (R)-1-(7-(8-ethyl-7-fluoro-3-hydroxynaphthalen-1-yl)-8-fluoro-2-(((3S,7aR)-3-(fluoromethyl)tetrahydro-1H-pyrrolizin-7a(5H)-yl)methoxy)pyrido[4,3-d]pyrimidin-4-yl)-3-methylpiperidin-3-ol

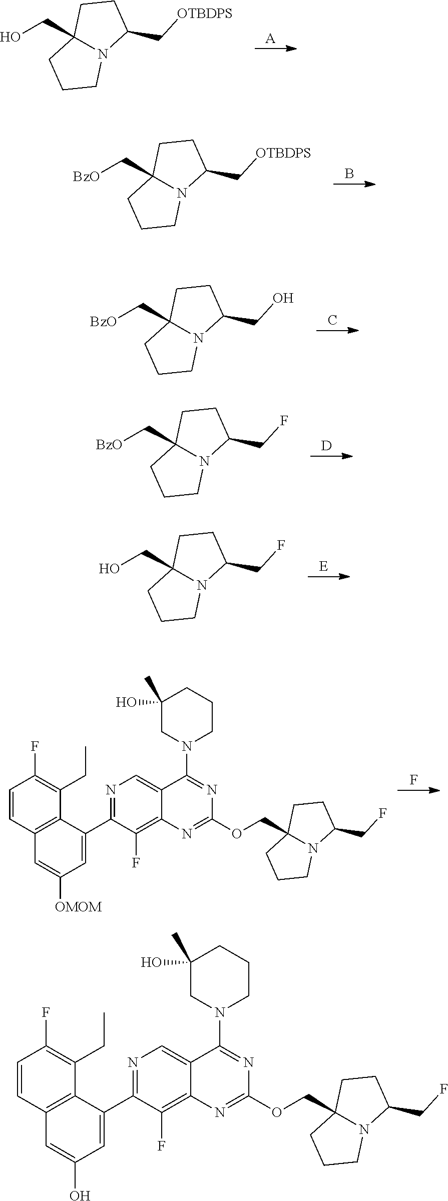

Step A. ((3S,7aR)-3-(((tert-butyldiphenylsilyl)oxy)methyl) hexahydro-1H-pyrrolizin-7a-yl)methyl benzoate: To a solution of ((3S,7aR)-3-(((tert-butyldiphenylsilyl)oxy)methyl) hexahydro-1H-pyrrolizin-7a-yl)methanol (3.00 g, 1.0 equiv) and TEA (1.48 g, 2.0 equiv) in DCM (30 mL) was added B₂Cl (1.55 g, 1.5 equiv) at 0° C. The reaction was stirred at 25° C. for 12 hours. The mixture was diluted with DCM (100 mL), dried over sodium sulfate, concentrated and purified by flash silica gel chromatography (ISCO®; 40 g SepaFlash® Silica Flash Column, Eluent of 0˜30% Ethyl acetate/Petroleum ethergradient@80 mL/min) to afford the title compound (2.40 g, 64% yield) as a yellow oil.

Step B. ((3S,7aR)-3-(hydroxymethyl) hexahydro-1H-pyrrolizin-7a-yl)methyl benzoate: To a solution of ((3S,7aR)-3-(((tert-butyldiphenylsilyl)oxy)methyl) hexahydro-1H-pyrrolizin-7a-yl)methyl benzoate (2.40 g, 1.0 equiv) in DMF (240 mL) was added CsF (10.6 g, 15.0 equiv). The reaction was stirred at 40° C. for 12 hours. The mixture was concentrated and purified by reversed phase flash chromatography [C18, 0.1% formic acid condition] to afford the title compound (1.04 g, 66% yield) as a yellow oil.

Step C. ((3S,7aR)-3-(fluoromethyl) hexahydro-1H-pyrrolizin-7a-yl)methyl benzoate: To a solution of ((3S,7aR)-3-(hydroxymethyl) hexahydro-1H-pyrrolizin-7a-yl)methyl benzoate (500 mg, 1.0 equiv) in DCM (15 mL) was added MOST (954 mg, 3.0 equiv) at 0° C. carefully. The reaction was stirred at 20° C. for 72 hours. The mixture was diluted with ice-cold sat. NaHCO₃ aqueous solution (20 mL) and extracted with DCM (3×10 mL). The combined organic layers were dried over sodium sulfate, concentrated and purified by reversed phase flash chromatography [C18, 0.1% formic acid condition], followed by SFC separation [column: DAICEL CHIRALPAK AD, 250×30 mm, 10 μm; A: CO2, B: MeOH (0.1% NH₃H₂O), B %: 20% B over 4.6 min] to afford the title compound (75.0 mg, 27% yield) as a yellow oil; LCMS (ESI, M+1): m/z=278.2.

Step D. ((3S,7aR)-3-(fluoromethyl) hexahydro-1H-pyrrolizin-7a-yl)methanol: To a solution of ((3S,7aR)-3-(fluoromethyl) hexahydro-1H-pyrrolizin-7a-yl)methyl benzoate (160 mg, 1.0 equiv) in MeOH (1.5 mL) was added NaOMe methanol solution (5 M, 231 μL, 2.0 equiv). The reaction was stirred at 0° C. for 0.5 hour. The mixture was quenched with water (1 mL), and then concentrated to afford the title compound (40.0 mg, crude) as a yellow oil.

Step E. (R)-1-(7-(8-ethyl-7-fluoro-3-(methoxymethoxy)naphthalen-1-yl)-8-fluoro-2-(((3S,7aR)-3-(fluoromethyl) hexahydro-1H-pyrrolizin-7a-yl)methoxy)pyrido[4,3-d]pyrimidin-4-yl)-3-methylpiperidin-3-ol: To a solution of ((3S,7aR)-3-(fluoromethyl) hexahydro-1H-pyrrolizin-7a-yl)methanol (20.0 mg, 1.0 equiv) and (3R)-1-[2-chloro-7-[8-ethyl-7-fluoro-3-(methoxymethoxy)-1-naphthyl]-8-fluoro-pyrido[4,3-d]pyrimidin-4-yl]-3-methyl-piperidin-3-ol (48.9 mg, 0.8 equiv) in toluene (0.5 mL) was added t-BuONa (22.2 mg, 2.0 equiv). The reaction was stirred at 0° C. for 1 hour. The mixture was concentrated to afford the title compound (75.0 mg, crude) as a yellow solid.

Step F. (R)-1-(7-(8-ethyl-7-fluoro-3-hydroxynaphthalen-1-yl)-8-fluoro-2-(((3S,7aR)-3-(fluoromethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethyl-7-fluoro-3-(methoxymethoxy)naphthalen-1-yl)-8-fluoro-2-(((3S,7aR)-3-(fluoromethyl) hexahydro-1H-pyrrolizin-7a-yl)methoxy)pyrido[4,3-d]pyrimidin-4-yl)-3-methylpiperidin-3-ol (75.0 mg, 1.0 equiv) in MeOH (0.5 mL) was added HCl/MeOH (2 M, 0.5 mL, 8.9 equiv). The reaction was stirred at 0° C. for 2 hours. The mixture was concentrated under reduced pressure and purified by prep-HPLC [column: Phenomenex luna C18 75×30 mm×3 μm; A: water (FA), B: ACN, B %: 15%-45% over 7 min] to afford the title compound (3.62 mg, 5% yield) as a yellow solid. ¹H NMR (400 MHZ, METHANOL-d₄) δ=9.22 (s, 1H), 7.68 (dd, J=6.0, 9.2 Hz, 1H), 7.31 (d, J=2.4 Hz, 1H), 7.25 (t, J=9.6 Hz, 1H), 7.06 (s, 1H), 4.92 (br s, 1H), 4.65-4.46 (m, 3H), 4.44-4.25 (m, 3H), 3.70-3.58 (m, 1H), 3.53-3.40 (m, 1H), 3.29-3.07 (m, 2H), 3.07-2.91 (m, 1H), 2.57-2.36 (m, 1H), 2.35-2.09 (m, 3H), 2.09-1.90 (m, 4H), 1.89-1.75 (m, 5H), 1.29 (d, J=10.4 Hz, 3H), 0.81 (q, J=7.2 Hz, 3H); LCMS (ESI, M+1): m/z=622.4.

### Example 880

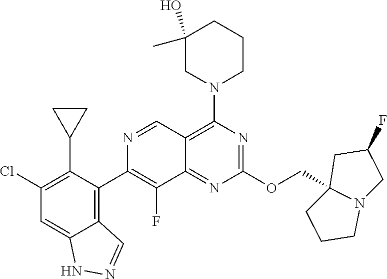 [SMILES: C[C@@]1(O)CCCN(c2nc(OC[C@@]34CCCN3C[C@H](F)C4)nc3c(F)c(-c4c(C5CC5)c(Cl)cc5[nH]ncc45)ncc23)C1]

### (3R)-1-(7-(6-chloro-5-cyclopropyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol

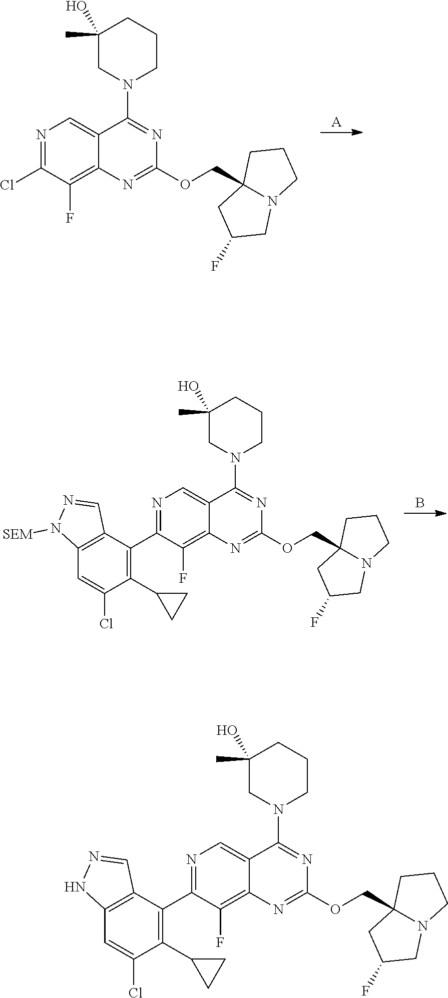

Step A. (3R)-1-(7-(6-chloro-5-cyclopropyl-1-((2-(trimethylsilyl) ethoxy)methyl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol: To a mixture of (R)-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (200 mg, 1.0 equiv) and 6-chloro-5-cyclopropyl-4-(4,4,5,5-tetramethyl-1,3,2-dioxaborolan-2-yl)-1-((2-(trimethylsilyl) ethoxy)methyl)-1H-indazole (237 mg, 1.2 equiv) in toluene (2 mL) and water (0.5 mL) were added APhos Pd G3 (42.0 mg, 0.15 equiv) and K₂CO₃ (280 mg, 3.0 equiv). The reaction was degassed and purged with nitrogen 3 times. The reaction was stirred at 60° C. for 12 hours. The mixture was diluted with H₂O (5 mL) and extracted with ethyl acetate (3×15 mL). The combined organic layers were dried over anhydrous sodium sulfate, concentrated and purified with reversed phase flash chromatography [C18, 0.1% formic acid condition] to afford the title compound (150 mg, 45% yield) as white solid; LCMS (ESI, M+1): m/z=740.5.

Step B. (3R)-1-(7-(6-chloro-5-cyclopropyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol: To a solution of (3R)-1-(7-(6-chloro-5-cyclopropyl-1-((2-(trimethylsilyl) ethoxy)methyl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (120 mg, 1.0 equiv) in dichloromethane (1 mL) was added 2,2,2-trifluoroacetic acid (164 mg, 10 equiv). The reaction was stirred at 25° C. for 12 hours. The mixture was concentrated. The residue as diluted with water (2 mL). The mixture was adjusted to pH=10 with NaOH solution (10% w/w) at 0° C. and extracted with dichloromethane (3×2 mL). The combined organic layers were dried over anhydrous sodium sulfate, concentrated, and purified with prep-HPLC [Phenomenex luna C18 150×25 mm×10 μm; A: water (FA), B: ACN, B %: 13%-43% over 10 min] to afford the title compound (26.5 mg, 27% yield, HCOOH salt) as white solid; ¹H NMR (400 MHZ, METHANOL-d₄) δ=9.33 (d, J=6.4 Hz, 1H), 7.79 (d, J=12.4 Hz, 2H), 5.49-5.27 (m, 1H), 5.44-4.57 (m, 1H), 4.46-4.29 (m, 3H), 3.64 (dd, J=13.6, 17.2 Hz, 1H), 3.53-3.34 (m, 4H), 3.13, (J=5.4, 9.8 Hz, 1H), 2.55-1.95 (m, 8H), 1.94-1.75 (m, 3H), 1.31 (d, J=8.0 Hz, 3H), 0.95-0.83 (m, 1H), 0.69-0.56 (m, 1H), 0.27-0.24 (m, 1H), 0.13-0.03 (m, 1H); LCMS (ESI, M+1): m/z=610.4.

### Example 881

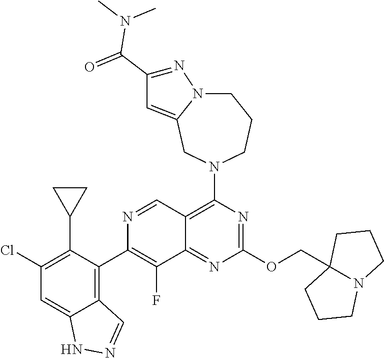 [SMILES: CN(C)C(=O)c1cc2n(n1)CCCN(c1nc(OCC34CCCN3CCC4)nc3c(F)c(-c4c(C5CC5)c(Cl)cc5[nH]ncc45)ncc13)C2]

### 5-(7-(6-chloro-5-cyclopropyl-1H-indazol-4-yl)-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

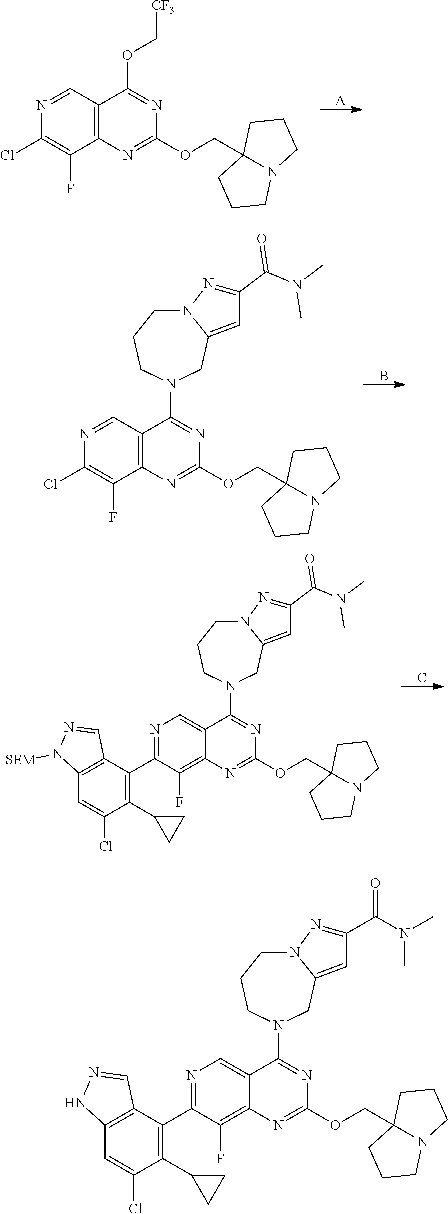

Step A. 5-(7-chloro-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 7-chloro-8-fluoro-2-((tetrahydro-1H-pyrrolizin-7a(5H)-yl)methoxy)-4-(2,2,2-trifluoroethoxy)pyrido[4,3-d]pyrimidine (1.00 g, 1.0 equiv) and N,N-dimethyl-5,6,7,8-tetrahydro-4H-pyrazolo[1,5-a][1,4]diazepine-2-carboxamide (742 mg, 1.5 equiv) in DMF (3 mL) was added DIEA (921 mg, 3.0 equiv). The reaction was stirred at 40° C. for 12 hours. The mixture was filtered and purified with reversed phase flash chromatography [C18, 0.1% formic acid condition] to afford the title compound (400 mg, 32% yield) as yellow solid; LCMS (ESI, M+1): m/z=529.3.

Step B. 5-(7-(6-chloro-5-cyclopropyl-1-((2-(trimethylsilyl) ethoxy)methyl)-1H-indazol-4-yl)-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a mixture of 5-(7-chloro-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (200 mg, 1.0 equiv) and 6-chloro-5-cyclopropyl-4-(4,4,5,5-tetramethyl-1,3,2-dioxaborolan-2-yl)-1-((2-(trimethylsilyl) ethoxy)methyl)-1H-indazole (204 mg, 1.2 equiv) in toluene (2.0 mL) and H₂O (756 μL) was added Aphos-Pd-G3 (27.5 mg, 0.10 equiv) and K₃PO₄ (241 mg, 3 equiv). The reaction was degassed and purged with nitrogen 3 times. The reaction was stirred at 60° C. for 12 hours. The mixture was diluted with water (5 mL) and extracted with EtOAc (4×10 mL). The combined organic layers were washed with brine (10 mL), dried over anhydrous sodium sulfate, concentrated and purified with column chromatography [SiO₂, petroleum ether/ethyl acetate=5/1 to dichloromethane/methanol=5/1] and reversed phase flash chromatography [C18, 0.1% formic acid condition] to afford the title compound (100 mg, 29% yield) as yellow solid; LCMS (ESI, M+1): m/z=815.3.

Step C 5-(7-(6-chloro-5-cyclopropyl-1H-indazol-4-yl)-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 5-(7-(6-chloro-5-cyclopropyl-1-((2-(trimethylsilyl) ethoxy)methyl)-1H-indazol-4-yl)-8-fluoro-2-((tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (80 mg, 1.0 equiv) in DCM (0.5 mL) was added TFA (0.5 mL). The reaction was stirred at 0° C. for 0.5 hours. The mixture was quenched with saturated NaHCO₃ aqueous (5 mL) at 0° C. and extracted with EtOAc (4×5 mL). The combined organic layers were washed with brine (10 mL), dried over anhydrous sodium sulfate and concentrated to give a residue. The residue was dissolved in THF (0.2 mL). NH₃·H₂O (0.4 mL, 25% purity) was added. The reaction was stirred at 20° C. for 4 hours. The mixture was diluted with water (2 mL) and extracted with EtOAc (3×3 mL). The combined organic layers were dried over anhydrous sodium sulfate, concentrated and purified with prep-HPLC [Phenomenex luna C18 150×25 mm×10 μm; A: water (FA), B: ACN; B %: 10%-40%, over 10 min] to afford the title compound (29.5 mg, 50% yield, 0.50 HCOOH) as white solid; ¹H NMR (400 MHZ, METHANOL-d₄) δ=9.31 (s, 1H), 7.81 (s, 1H), 7.78 (s, 1H), 6.79 (s, 1H), 5.38-5.22 (m, 2H), 4.60 (br s, 2H), 4.53-4.43 (m, 4H), 3.45-3.37 (m, 2H), 3.34 (s, 3H), 3.08 (s, 3H), 3.06-2.97 (m, 2H), 2.47 (br d, J=5.2 Hz, 2H), 2.26-2.15 (m, 2H), 2.12-1.99 (m, 5H), 1.98-1.89 (m, 2H), 0.93-0.86 (m, 1H), 0.64-0.57 (m, 1H), 0.30-0.20 (m, 1H), 0.11-0.01 (m, 1H); LCMS (ESI, M+1): m/z=685.2.

### Example 882

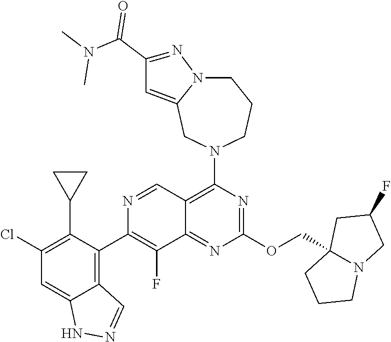 [SMILES: CN(C)C(=O)c1cc2n(n1)CCCN(c1nc(OC[C@@]34CCCN3C[C@H](F)C4)nc3c(F)c(-c4c(C5CC5)c(Cl)cc5[nH]ncc45)ncc13)C2]

### 5-(7-(6-chloro-5-cyclopropyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

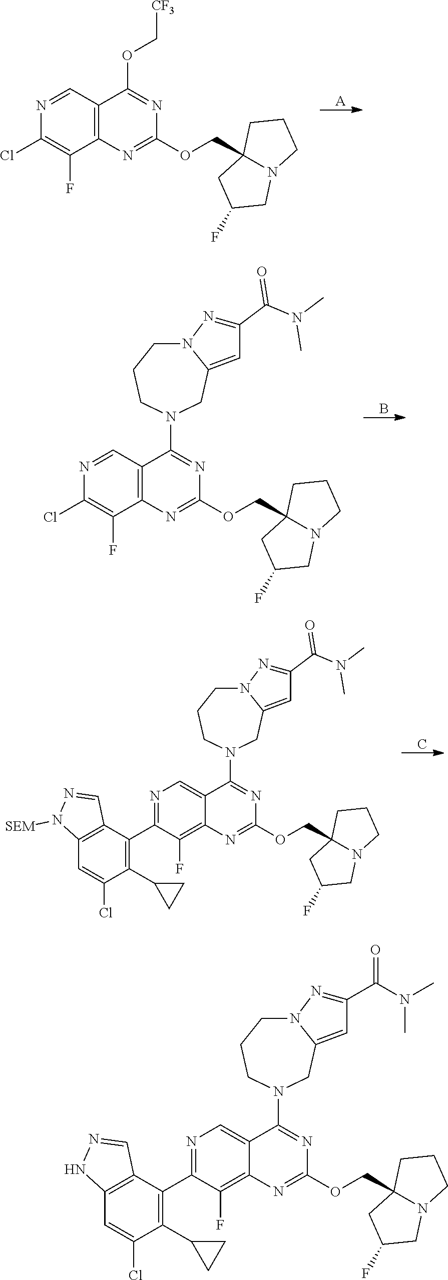

Step A. 5-(7-chloro-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (500 mg, 1.0 equiv) and N,N-dimethyl-5,6,7,8-tetrahydro-4H-pyrazolo[1,5-a][1,4]diazepine-2-carboxamide (356 mg, 1.5 equiv) in DMF (1.5 mL) was added DIEA (442 mg, 3.0 equiv). The reaction was stirred at 40° C. for 12 hours. The mixture was filtered and purified with reversed phase flash chromatography [C18, 0.1% formic acid condition]. to afford the title compound (500 mg, 80% yield) as yellow solid; LCMS (ESI, M+1): m/z=547.3.

Step B. 5-(7-(6-chloro-5-cyclopropyl-1-((2-(trimethylsilyl) ethoxy)methyl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a mixture of 5-(7-chloro-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (200 mg, 1.0 equiv) and 6-chloro-5-cyclopropyl-4-(4,4,5,5-tetramethyl-1,3,2-dioxaborolan-2-yl)-1-((2-(trimethylsilyl) ethoxy)methyl)-1H-indazole (197 mg, 1.2 equiv) in toluene (2 mL) and H₂O (731 μL) were added Aphos-Pd-G3 (26.6 mg, 0.10 equiv) and K₃PO₄ (233 mg, 3 equiv). The reaction was degassed and purged with nitrogen 3 times. The reaction was stirred at 60° C. for 12 hours. The mixture was diluted with water (5 mL) and extracted with EtOAc (4×10 mL). The combined organic layers were washed with brine (10 mL), dried over anhydrous sodium sulfate, concentrated and purified with column chromatography [SiO₂, petroleum ether/ethyl acetate=5/1 to dichloromethane/methanol=5/1] and reversed phase flash chromatography [C18, 0.1% formic acid condition] to afford the title compound (100 mg, 30% yield) as yellow solid; LCMS (ESI, M+1): m/z=833.4.

Step C. 5-(7-(6-chloro-5-cyclopropyl-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 5-(7-(6-chloro-5-cyclopropyl-1-((2-(trimethylsilyl) ethoxy)methyl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (80 mg, 1.0 equiv) in DCM (0.5 mL) was added TFA (0.5 mL). The reaction was stirred at 0° C. for 0.5 hours. The mixture was quenched with saturated NaHCO₃ aqueous (5 mL) at 0° C. and extracted with EtOAc (4×5 mL). The organic phase was dried over anhydrous sodium sulfate and concentrated. The residue was diluted with THF (0.2 mL) and NH₃·H₂O (0.4 mL 25% purity). The reaction was stirred at 20° C. for 12 hours. The mixture was diluted with water (2 mL) and extracted with EtOAc (3×3 mL). The combined organic layers were washed with brine (5 mL), dried over anhydrous sodium sulfate, concentrated and purified with prep-HPLC [Phenomenex luna C18 150×25 mm×10 μm; A: water (NH₄HCO₃), B: ACN; B %: 34%-64%, over 10 min] to afford the title compound (58.5 mg, 76% yield) as white solid; ¹H NMR (400 MHZ, METHANOL-d₄) δ=9.27 (s, 1H), 7.81 (s, 1H), 7.77 (d, J=1.2 Hz, 1H), 6.78 (s, 1H), 5.41-5.20 (m, 3H), 4.59-4.54 (m, 2H), 4.53-4.38 (m, 2H), 4.34-4.20 (m, 2H), 3.34 (s, 3H), 3.30-3.14 (m, 3H), 3.08 (s, 3H), 3.03-2.99 (m, 1H), 2.52-2.40 (m, 2H), 2.36-2.24 (m, 1H), 2.23-2.17 (m, 1H), 2.17-2.10 (m, 1H), 2.07-1.95 (m, 3H), 1.95-1.83 (m, 1H), 0.96-0.81 (m, 1H), 0.66-0.53 (m, 1H), 0.32-0.18 (m, 1H), 0.13-0.02 (m, 1H); LCMS (ESI, M+1): m/z=703.2.

### Example 883

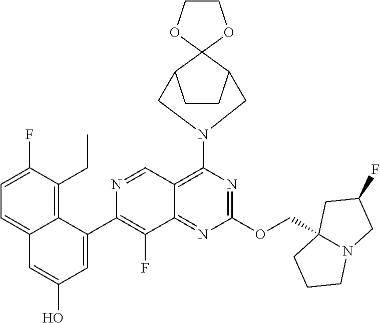 [SMILES: CCc1c(F)ccc2cc(O)cc(-c3ncc4c(N5CC6CCC(C5)C65OCCO5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(3-azaspiro[bicyclo[3.2.1]octane-8,2′-[1,3]dioxolan]-3-yl)pyrido[4,3-d]pyrimidin-7-yl)naphthalen-2-ol

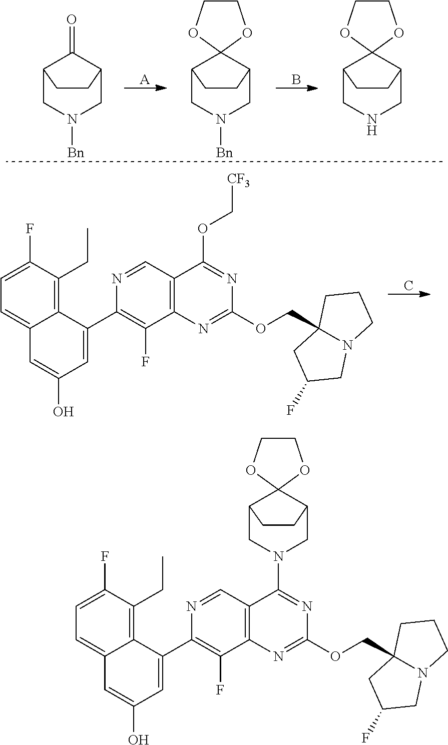

Step A. (1R,5S)-3-benzyl-3-azaspiro[bicyclo[3.2.1]octane-8,2′-[1,3]dioxolane]; To a solution of (1R,5S)-3-benzyl-3-azabicyclo[3.2.1]octan-8-one (300 mg, 1 equiv) and ethane-1,2-diol (86.5 mg, 1.0 equiv) in toluene (3 mL) was added TsOH·H₂O (26.5 mg, 0.1 equiv). The reaction was stirred at 100° C. for 12 hours. The mixture was diluted with H₂O (5 mL) and extracted with EtOAc (3×5 mL). The combined organic layers were washed with brine (10 mL), dried over anhydrous sodium sulfate, concentrated to afford the title compound (200 mg, 55% yield) as yellow oil.

Step B. (1R,5S)-3-azaspiro[bicyclo[3.2.1]octane-8,2′-[1,3]dioxolane]: To a solution of (1R,5S)-3-benzyl-3-azaspiro[bicyclo[3.2.1]octane-8,2′-[1,3]dioxolane] (200 mg, 1.0 equiv) and NH₃·MeOH (4 M, 0.2 mL) in MeOH (2 mL) was added Pd/C (50.0 mg, 10% purity) under nitrogen atmosphere. The reaction was degassed and purged with H₂ 3 times. The reaction was stirred under H₂ (15 Psi) at 20° C. for 1 hour. The mixture was filtered and concentrated under reduced pressure to afford the title compound (80 mg, 60% yield) as white solid.

Step C. 5-ethyl-6-fluoro-4-(8-fluoro-2-(((2R,7aS)-2-fluorotetrahydro-1H-pyrrolizin-7a(5H)-yl)methoxy)-4-((1R,5S)-3-azaspiro[bicyclo[3.2.1]octane-8,2′-[1,3]dioxolan]-3-yl)pyrido[4,3-d]pyrimidin-7-yl)naphthalen-2-ol: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (100 mg, 1.0 equiv) and (1R,5S)-3-azaspiro[bicyclo[3.2.1]octane-8,2′-[1,3]dioxolane] (42.8 mg, 1.5 equiv) in what solvent? were added DIEA (218 mg, 10 equiv) and 4 Å molecular sieve (10 mg). The reaction was stirred at 60° C. for 12 hours. The mixture was filtered and purified by prep-HPLC (column: Waters xbridge 150×25 mm×10 um; mobile phase: [water (ammonia hydroxide v/v)-ACN]; B %: 50%-80%, 10 min) to afford the title compound (5.48 mg, 51% yield) as white solid; ¹H NMR (400 MHZ, METHANOL-d₄) δ=9.21-9.03 (m, 1H), 7.67 (dd, J=6.0, 9.2 Hz, 1H), 7.32-7.21 (m, 2H), 7.08-7.02 (m, 1H), 5.39-5.22 (m, 1H), 4.75-4.64 (m, 3H), 4.34-4.29 (m, 1H), 4.27-4.21 (m, 1H), 4.11-3.96 (m, 5H), 3.26-3.16 (m, 3H), 3.01 (dt, J=6.0, 9.2 Hz, 1H), 2.55-2.44 (m, 1H), 2.35-2.05 (m, 6H), 2.03-1.84 (m, 5H), 1.56 (br d, J=9.6 Hz, 2H), 0.78-0.78 (m, 1H), 0.80 (dt, J=2.0, 7.2 Hz, 2H); LCMS (ESI, M−1): m/z=662.2.

### Example 884

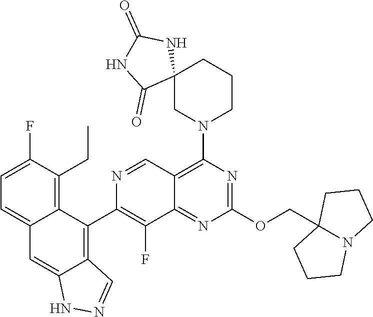 [SMILES: CCc1c(F)ccc2cc3[nH]ncc3c(-c3ncc4c(N5CCC[C@]6(C5)NC(=O)NC6=O)nc(OCC56CCCN5CCC6)nc4c3F)c12]

### (5R)-7-(7-(5-ethyl-6-fluoro-1H-benzo[f]indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione

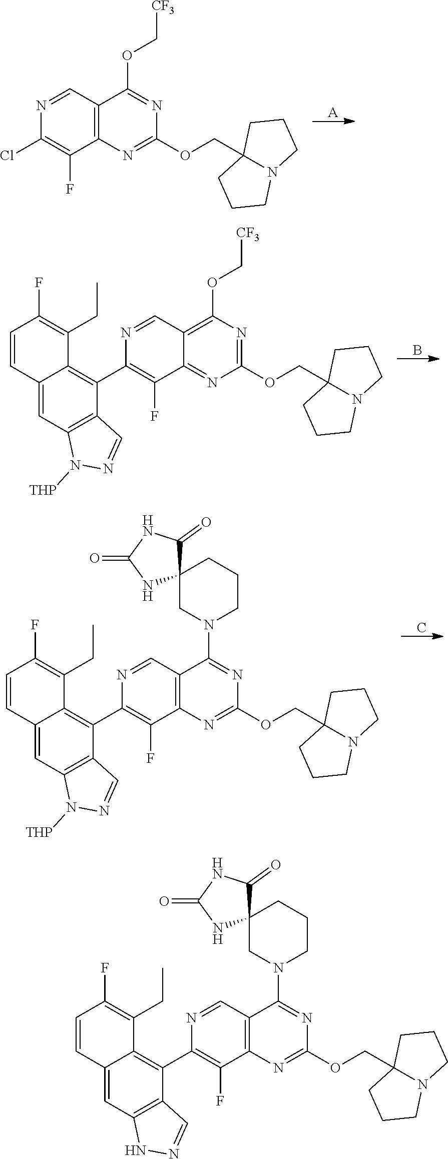

Step A. 5-ethyl-6-fluoro-4-(8-fluoro-2-((hexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole: To a solution of 7-chloro-8-fluoro-2-((hexahydro-1H-pyrrolizin-7a-yl)methoxy)-4-(2,2,2-trifluoroethoxy)pyrido[4,3-d]pyrimidine (50 mg, 1.0 equiv), 5-ethyl-6-fluoro-1-(tetrahydro-2H-pyran-2-yl)-4-(4,4,5,5-tetramethyl-1,3,2-dioxaborolan-2-yl)-1H-benzo[f]indazole (50.4 mg, 1.0 equiv) and Cs₂CO₃ (1.5 M, 3.0 equiv) in Methoxy cyclopentane (2 mL) was added CataCXium A Pd G3 (8.65 mg, 0.1 equiv). The reaction was degassed and purged with nitrogen 3 times. The reaction was stirred at 80° C. for 5 hours. The mixture was added H₂O (5 mL) and extracted with ethyl acetate (5 mL×3). The organic layer was dried over anhydrous sodium sulfate, concentrated, and purified with reversed phase flash chromatography (C18, 0.1% formic acid condition) to afford the title compound (17.0 mg, 19% yield) as yellow oil; LCMS (ESI, M+1): m/z=683.3.

Step B. (5R)-7-(7-(5-ethyl-6-fluoro-1-(tetrahydro-2H-pyran-2-yl)-1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a solution of 5-ethyl-6-fluoro-4-(8-fluoro-2-((hexahydro-1H-pyrrolizin-7a-yl)methoxy)-4-(2,2,2-trifluoroethoxy)pyrido[4,3-d]pyrimidin-7-yl)-1-(tetrahydro-2H-pyran-2-yl)-1H-benzo[f]indazole (15.0 mg, 1.0 equiv) and (R)-1,3,7-triazaspiro[4.5]decane-2,4-dione (15 mg, 4.04 equiv) in DMAc (1.5 mL) were added 4 Å molecular sieve (2.0 mg) and CsF (33.4 mg, 10.0 equiv). The reaction was stirred at 40° C. for 1.5 hours. The mixture was filtered and purified by reversed phase flash chromatography (C18, 0.1% formic acid condition) to afford the title compound (15 mg, 91% yield) as yellow oil; LCMS (ESI, M+1): m/z=752.6.

Step C. (5R)-7-(7-(5-ethyl-6-fluoro-1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione: To a solution of (5R)-7-(7-(5-ethyl-6-fluoro-1-(tetrahydro-2H-pyran-2-yl)-1H-benzo[f]indazol-4-yl)-8-fluoro-2-((hexahydro-1H-pyrrolizin-7a-yl)methoxy)pyrido[4,3-d]pyrimidin-4-yl)-1,3,7-triazaspiro[4.5]decane-2,4-dione (15.0 mg, 1.0 equiv) in DCM (0.5 mL) was added TFA (770 mg, 0.5 mL). The reaction was stirred at 20° C. for 0.5 hour. The mixture was concentrated at 25° C. The residue was diluted with water (1 mL). The mixture was adjusted to pH=7 with saturated NaHCO₃ (10 mL) and extracted with ethyl acetate (5 mL×3). The organic layer was dried over anhydrous sodium sulfate, concentrated, and purified with prep-HPLC (column: Phenomenex Luna C18 150×25 mm×10 um; mobile phase: [water (FA)-ACN]; B %: 11%-41% over 12 min) to afford the title compound (2.89 mg, 21% yield, 0.52 HCOOH) as yellow solid; ¹H NMR (400 MHZ, METHANOL-d₄) δ=9.25 (s, 1H), 8.26 (s, 1H), 8.03 (dd, J=6.0, 9.6 Hz, 1H), 7.70 (d, J=2.8 Hz, 1H), 7.34 (t, J=9.6 Hz, 1H), 4.74-4.63 (m, 2H), 4.53-4.47 (m, 2H), 3.96-3.79 (m, 2H), 3.46-3.40 (m, 2H), 3.10-2.96 (m, 2H), 2.70-2.56 (m, 1H), 2.43-2.34 (m, 1H), 2.32-2.16 (m, 4H), 2.14-2.03 (m, 5H), 2.01-1.92 (m, 3H), 0.92-0.80 (m, 3H); LCMS (ESI, M+1): m/z=668.3.

### Example 885

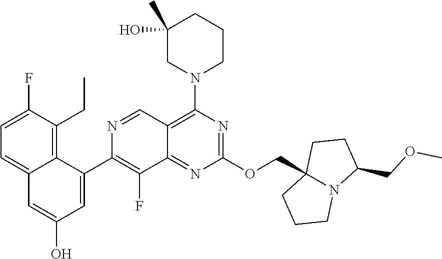 [SMILES: CCc1c(F)ccc2cc(O)cc(-c3ncc4c(N5CCC[C@@](C)(O)C5)nc(OC[C@]56CCCN5[C@H](COC)CC6)nc4c3F)c12]

### (R)-1-(7-(8-ethyl-7-fluoro-3-hydroxynaphthalen-1-yl)-8-fluoro-2-(((3S,7aR)-3-(methoxymethyl)tetrahydro-1H-pyrrolizin-7a(5H)-yl)methoxy)pyrido[4,3-d]pyrimidin-4-yl)-3-methylpiperidin-3-ol

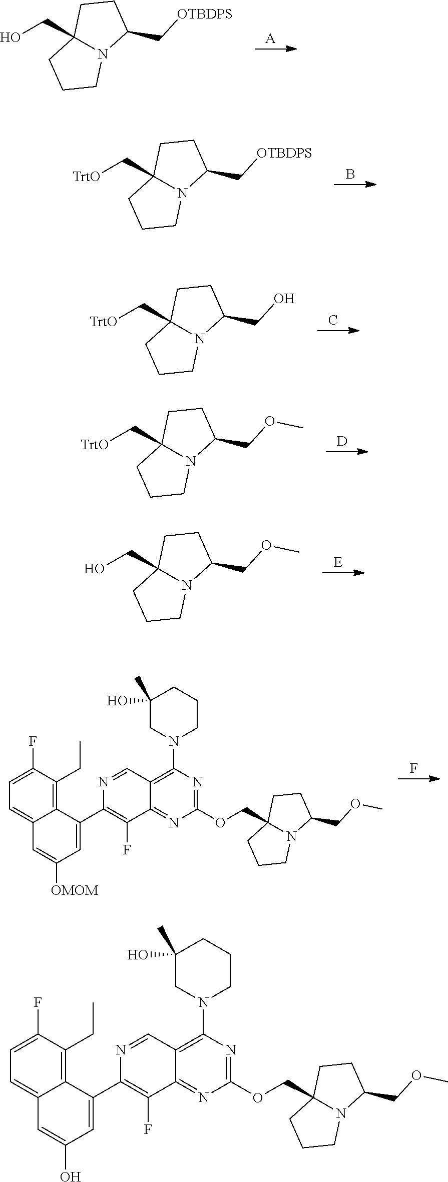

Step A. (3S,7aR)-3-(((tert-butyldiphenylsilyl)oxy)methyl)-7a-((trityloxy)methyl) hexahydro-1H-pyrrolizin: To a solution of ((3S,7aR)-3-(((tert-butyldiphenylsilyl)oxy)methyl) hexahydro-1H-pyrrolizin-7a-yl)methanol (9.00 g, 1.0 equiv) and TEA (4.45 g, 2.0 equiv) in DCM (100 mL) was added TrtCl (9.19 g, 1.5 equiv). The reaction was stirred at 25° C. for 12 hours. The mixture was diluted with water (50 mL) and extracted with DCM (3× 50 mL). The combined organic layers were washed with brine (3×50 mL), dried over sodium sulfate and concentrated to afford the title compound (12.0 g, crude) as a yellow oil.

Step B. ((3S,7aR)-7a-((trityloxy)methyl) hexahydro-1H-pyrrolizin-3-yl)methanol: To a solution of (3S,7aR)-3-(((tert-butyldiphenylsilyl)oxy)methyl)-7a-((trityloxy)methyl) hexahydro-1H-pyrrolizin (1.00 g, 1.0 equiv) in DMF (10 mL) was added CsF (2.33 g, 10 equiv). The reaction was stirred at 40° C. for 12 hours. The mixture was diluted with water (50 mL) and extracted with ethyl acetate (3×50 mL). The combined organic layers were washed with brine (3×50 mL), dried over sodium sulfate, concentrated and purified by reversed phase flash chromatography [C18, 0.1% formic acid condition] to afford the title compound (500 mg, 79% yield) as a yellow oil.

Step C. (3S,7aR)-3-(methoxymethyl)-7a-((trityloxy)methyl) hexahydro-1H-pyrrolizine: To a solution of ((3S,7aR)-7a-((trityloxy)methyl) hexahydro-1H-pyrrolizin-3-yl)methanol (200 mg, 1.0 equiv) in DMF (2 mL) was added NaH (38.7 mg, 60% purity, 2.0 equiv) at 0° C. The mixture was stirred at 0° C. for 30 minutes. MeI (103 mg, 1.5 equiv) was added. The reaction was stirred at 0° C. for 2 hours. The reaction was quenched by addition of ice-water (10 mL) and extracted with ethyl acetate (2×10 mL). The combined organic layers were washed with brine (10 mL), dried over sodium sulfate, concentrated and purified by prep-HPLC [column: Phenomenex luna C18 75×30 mm×3 μm; A: water (FA), B: ACN, B %: 27%-47% over 8 min] to afford the title compound (120 mg, 56% yield) as a yellow oil; LCMS (ESI, M+1): m/z=428.3.

Step D. ((3S,7aR)-3-(methoxymethyl) hexahydro-1H-pyrrolizin-7a-yl)methanol: To a solution of (3S,7aR)-3-(methoxymethyl)-7a-((trityloxy)methyl) hexahydro-1H-pyrrolizine (100 mg, 1.0 equiv) in DCM (0.8 mL) was added TFA (400 uL, 23.1 equiv). The reaction was stirred at 20° C. for 2 hours. The mixture was concentrated to afford the title compound (100 mg, crude) as a yellow solid.

Step E. (R)-1-(7-(8-ethyl-7-fluoro-3-(methoxymethoxy)naphthalen-1-yl)-8-fluoro-2-(((3S,7aR)-3-(methoxymethyl) hexahydro-1H-pyrrolizin-7a-yl)methoxy)pyrido[4,3-d]pyrimidin-4-yl)-3-methylpiperidin-3-ol: A mixture of ((3S,7aR)-3-(methoxymethyl) hexahydro-1H-pyrrolizin-7a-yl)methanol (29.8 mg, 1.0 equiv), 4 Å molecular sieve (5 mg) and t-BuONa (61.8 mg, 4.0 equiv) in toluene (1 mL) was stirred at 0° C. for 10 minutes. (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (85.0 mg, 1.0 equiv) was added. The reaction was stirred at 0° C. for 1 hour. The mixture was quenched with water (10 mL) at 0° C. and extracted with ethyl acetate (3×10 mL). The combined organic layers were washed with brine (20 mL), dried over sodium sulfate, concentrated under vacuum and purified by prep-HPLC [column: YMC Triart C18 150×25 mm×5 μm; A: water (FA), B: ACN, B %: 40%-60% over 8 min] to afford the title compound (30.0 mg, 27% yield) as a white solid; LCMS (ESI, M+1): m/z=678.5.

Step F. (R)-1-(7-(8-ethyl-7-fluoro-3-hydroxynaphthalen-1-yl)-8-fluoro-2-(((3S,7aR)-3-(methoxymethyl) hexahydro-1H-pyrrolizin-7a-yl)methoxy)pyrido[4,3-d]pyrimidin-4-yl)-3-methylpiperidin-3-ol: To of (R)-1-(7-(8-ethyl-7-fluoro-3-a solution (methoxymethoxy)naphthalen-1-yl)-8-fluoro-2-(((3S,7aR)-3-(methoxymethyl) hexahydro-1H-pyrrolizin-7a-yl)methoxy)pyrido[4,3-d]pyrimidin-4-yl)-3-methylpiperidin-3-ol (20.0 mg, 1.0 equiv) in MeOH (0.2 mL) was added HCl/MeOH (4 M, 0.2 mL) at 0° C. The reaction was stirred at 25° C. for 0.5 hours. The mixture was concentrated, diluted with MeCN (0.5 mL) and adjusted to pH=˜ 7 with 10% NH₃·H₂O. The mixture was directly purified by prep-HPLC [column: YMC Triart C18 150×25 mm×5 μm; A: water (FA), B: ACN, B %: 20%-50% over 8.5 min] to afford the title compound (14.0 mg, 74% yield) as a white solid. ¹H NMR (400 MHZ, METHANOL-d₄) δ=9.26 (s, 1H), 8.56 (br d, J=2.0 Hz, 1H), 7.70 (dd, J=5.9, 9.0 Hz, 1H), 7.33 (d, J=2.7 Hz, 1H), 7.27 (t, J=9.4 Hz, 1H), 7.08 (s, 1H), 4.61 (br s, 5H), 4.44 (br s, 1H), 4.33 (br t, J=14.8 Hz, 1H), 3.72-3.58 (m, 1H), 3.57-3.42 (m, 3H), 3.34 (br s, 3H), 2.54-2.42 (m, 1H), 2.39-1.74 (m, 13H), 1.31 (d, J=10.0 Hz, 3H), 0.88-0.77 (m, 3H); LCMS (ESI, M+1): m/z=634.3.

### Example 886

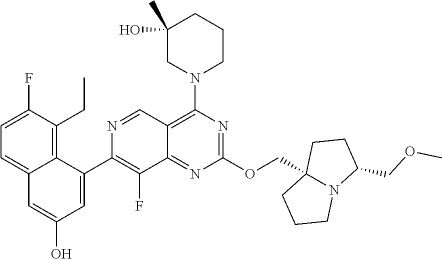 [SMILES: CCc1c(F)ccc2cc(O)cc(-c3ncc4c(N5CCC[C@@](C)(O)C5)nc(OC[C@@]56CCCN5[C@@H](COC)CC6)nc4c3F)c12]

### (R)-1-(7-(8-ethyl-7-fluoro-3-hydroxynaphthalen-1-yl)-8-fluoro-2-(((3R,7aS)-3-(methoxymethyl)tetrahydro-1H-pyrrolizin-7a(5H)-yl)methoxy)pyrido[4,3-d]pyrimidin-4-yl)-3-methylpiperidin-3-ol

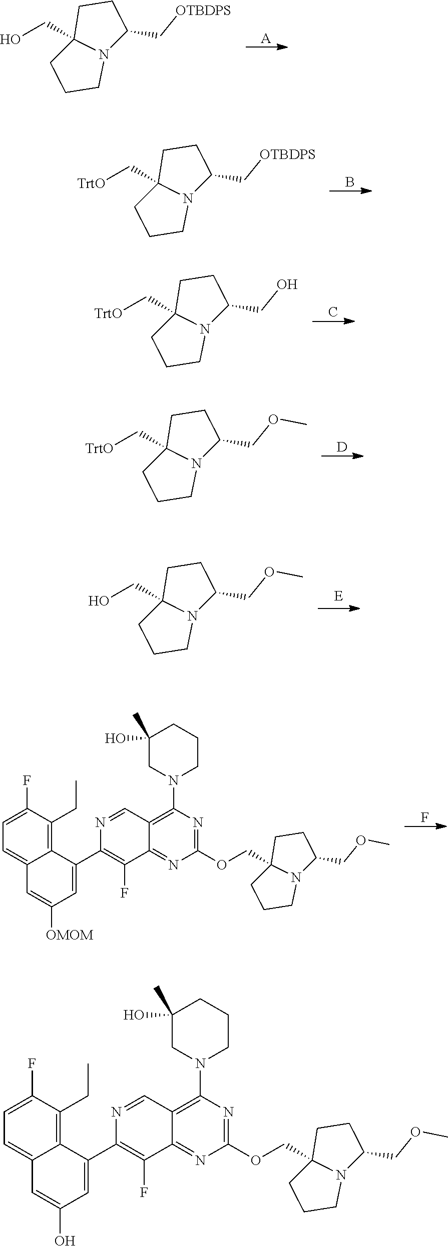

Step A. (3R,7aS)-3-(((tert-butyldiphenylsilyl)oxy)methyl)-7a-((trityloxy)methyl) hexahydro-1H-pyrrolizine: To a solution of ((3R,7aS)-3-(((tert-butyldiphenylsilyl)oxy)methyl) hexahydro-1H-pyrrolizin-7a-yl)methanol (6.00 g, 1.0 equiv) and TEA (2.96 g, 2.0 equiv) in DCM (60 mL) was added TrtCl (6.12 g, 1.5 equiv). The reaction was stirred at 25° C. for 12 hours. The mixture was washed with water (50 mL) and extracted with DCM (2× 30 mL). The combined organic layers were dried over sodium sulfate and concentrated to afford the title compound (9.00 g, crude) as a yellow oil.

Step B. ((3R,7aS)-7a-((trityloxy)methyl) hexahydro-1H-pyrrolizin-3-yl)methanol: To a solution of (3R,7aS)-3-(((tert-butyldiphenylsilyl)oxy)methyl)-7a-((trityloxy)methyl) hexahydro-1H-pyrrolizine (9.00 g, 1.0 equiv) in DMF (90 mL) was added CsF (31.5 g, 15 equiv). The reaction was stirred at 40° C. for 12 hours. The mixture was filtered and the filtrate was directly purified by reversed-phase HPLC (0.1% FA condition) to afford the title compound (4.00 g, 70% yield) as a yellow oil.

Step C. (3R,7aS)-3-(methoxymethyl)-7a-((trityloxy)methyl) hexahydro-1H-pyrrolizine: To a solution of ((3R,7aS)-7a-((trityloxy)methyl) hexahydro-1H-pyrrolizin-3-yl)methanol (100 mg, 1.0 equiv) in DMF (1 mL) was added NaH (19.3 mg, 60% purity, 2.0 equiv). The mixture was stirred at 0° C. for 30 minutes. MeI (34.3 mg, 1.0 equiv) was added. The reaction was stirred at 25° C. for 12 hours. The mixture was quenched by addition of ice-water (10 mL) and extracted with ethyl acetate (2×10 mL). The combined organic layers were washed with brine (10 mL), dried over sodium sulfate, concentrated and purified by prep-HPLC [column: Phenomenex luna C18 75×30 mm×3 μm; A: water (FA), B: ACN, B %: 22%-52% over 7 min] to afford the title compound (50.0 mg, 47% yield) as a white solid; LCMS (ESI, M+1): m/z=428.4.

Step D. ((3R,7aS)-3-(methoxymethyl) hexahydro-1H-pyrrolizin-7a-yl)methanol: To a solution of (3R,7aS)-3-(methoxymethyl)-7a-((trityloxy)methyl) hexahydro-1H-pyrrolizine (50.0 mg, 1.0 equiv) in DCM (0.4 mL) was added TFA (200 uL, 23.1 equiv). The reaction was stirred at 20° C. for 12 hours. The mixture was concentrated to afford the title compound (50.0 mg, crude) as a yellow solid.

Step E. (R)-1-(7-(8-ethyl-7-fluoro-3-(methoxymethoxy)naphthalen-1-yl)-8-fluoro-2-(((3R,7aS)-3-(methoxymethyl) hexahydro-1H-pyrrolizin-7a-yl)methoxy)pyrido[4,3-d]pyrimidin-4-yl)-3-methylpiperidin-3-ol: A mixture of ((3R,7aS)-3-(methoxymethyl) hexahydro-1H-pyrrolizin-7a-yl)methanol (38.5 mg, 1.0 equiv), 4 Å molecular sieve (2 mg) and t-BuONa (79.9 mg, 4.0 equiv) in toluene (1 mL) was stirred at 0° C. for 10 minutes. (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (110 mg, 1.0 equiv) was added. The reaction was stirred at 0° C. for 1 hour. The mixture was quenched with H₂O (10 mL) at 0° C. and extracted with ethyl acetate (3×10 mL). The combined organic layers were washed with brine (20 mL), dried over sodium sulfate, concentrated and purified by prep-HPLC [column: YMC Triart C18 150×25 mm×5 μm; A: water (FA), B: ACN, B %: 24%-54% over 8 min] to afford the title compound (25.0 mg, 17% yield) as a white solid; LCMS (ESI, M+1): m/z=678.5.

Step F. (R)-1-(7-(8-ethyl-7-fluoro-3-hydroxynaphthalen-1-yl)-8-fluoro-2-(((3R,7aS)-3-(methoxymethyl) hexahydro-1H-pyrrolizin-7a-yl)methoxy)pyrido[4,3-d]pyrimidin-4-yl)-3-methylpiperidin-3-ol: To a solution of (R)-1-(7-(8-ethyl-7-fluoro-3-(methoxymethoxy)naphthalen-1-yl)-8-fluoro-2-(((3R,7aS)-3-(methoxymethyl) hexahydro-1H-pyrrolizin-7a-yl)methoxy)pyrido[4,3-d]pyrimidin-4-yl)-3-methylpiperidin-3-ol (15.0 mg, 1.0 equiv) in MeOH (0.15 mL) was added HCl/MeOH (4 M, 300 uL) at 0° C. The mixture was stirred at 25° C. for 0.5 hour. The mixture was concentrated under reduced pressure at 30° C., diluted with MeCN (0.5 mL) and adjusted to pH=˜ 7 with 10% NH₃·H₂O. The mixture was directly purified by prep-HPLC [column: Phenomenex luna C18 150×25 mm×5 μm; A: water (FA), B: ACN, B %: 47%-54% over 8 min] prep-HPLC (column: Phenomenex luna C18 150*25 mm*10 um; mobile phase: [water (FA)-ACN]; B %: 17%-47%, min) to afford the title compound (8.00 mg, 55% yield) as a white solid. ¹H NMR (400 MHZ, METHANOL-d₄) δ=9.24 (d, J=5.6 Hz, 1H), 8.64-8.48 (m, 1H), 7.70 (dd, J=6.0, 8.8 Hz, 1H), 7.33 (d, J=2.8 Hz, 1H), 7.27 (t, J=9.2 Hz, 1H), 7.13-7.00 (m, 1H), 4.61 (br s, 3H), 4.45-4.28 (m, 3H), 3.71-3.57 (m, 1H), 3.50 (br d, J=5.2 Hz, 3H), 3.36 (d, J=6.8 Hz, 3H), 3.24-3.05 (m, 2H), 2.48 (qd, J=7.2, 14.4 Hz, 1H), 2.35-2.13 (m, 3H), 2.12-1.95 (m, 4H), 1.94-1.75 (m, 6H), 1.31 (d, J=9.2 Hz, 3H), 0.90-0.77 (m, 3H); LCMS (ESI, M+1): m/z=634.3.

### Example 887

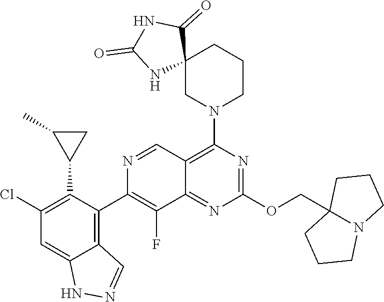 [SMILES: C[C@@H]1C[C@@H]1c1c(Cl)cc2[nH]ncc2c1-c1ncc2c(N3CCC[C@]4(C3)NC(=O)NC4=O)nc(OCC34CCCN3CCC4)nc2c1F]

### (R)-7-(7-((R)-6-chloro-5-((1S,2R)-2-methylcyclopropyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione

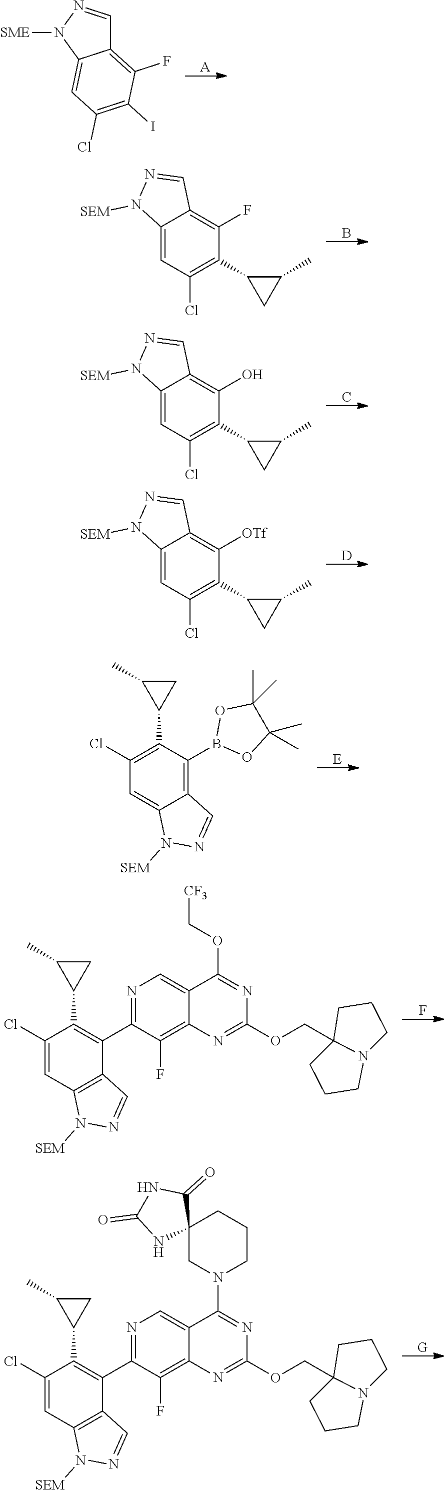

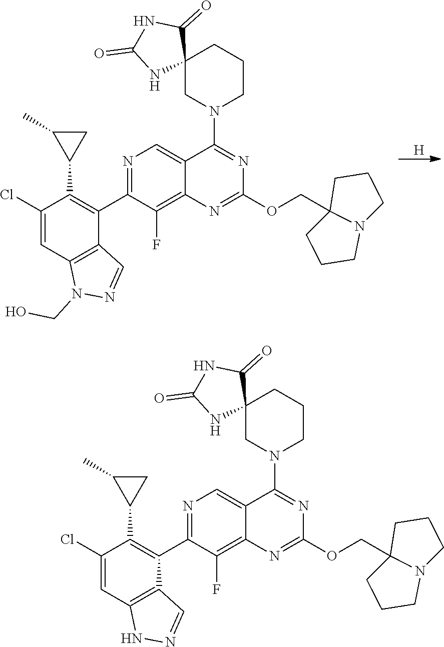

Step A. 6-chloro-4-fluoro-5-((1S,2R)-2-methylcyclopropyl)-1-((2-(trimethylsilyl) ethoxy)methyl)-1H-indazole: To a mixture of 6-chloro-4-fluoro-5-iodo-1-((2-(trimethylsilyl) ethoxy)methyl)-1H-indazole (10.0 g, 1.0 equiv) and 4,4,5,5-tetramethyl-2-((1S,2R)-2-methylcyclopropyl)-1,3,2-dioxaborolane (8.53 g, 2.0 equiv) in dioxane (75 mL) and H₂O (25 mL) were added K₃PO₄ (14.9 g, 3.0 equiv) and Pd(dppf)Cl₂ (1.71 g, 0.1 equiv). The reaction was degassed and purged with nitrogen 3 times. The reaction was stirred at 100° C. for 12 hours. The mixture was diluted with water (40 mL) and extracted with ethyl acetate (3×30 mL). The combined organic layers were dried over anhydrous sodium sulfate, concentrated and purified by reversed phase flash chromatography [C18, 0.1% formic acid condition] to afford the title compound (4.70 g, 54% yield) as yellow liquid; LCMS (ESI, M+1): m/z=355.0.

Step B. 6-chloro-5-((1S,2R)-2-methylcyclopropyl)-1-((2-(trimethylsilyl) ethoxy)methyl)-1H-indazol-4-ol: To a solution of 6-chloro-4-fluoro-5-((1S,2R)-2-methylcyclopropyl)-1-((2-(trimethylsilyl) ethoxy)methyl)-1H-indazole (3.00 g, 1.0 equiv) and 2-(methylsulfonyl) ethan-1-ol (4.20 g, 4.0 equiv) in DMAc (30 mL) was added NaH (2.03 g, 60% purity, 6.0 equiv) portion-wise at 0° C. The reaction was stirred at 40° C. for 12 hours. The mixture was diluted with water (40 mL) and extracted with ethyl acetate (3×30 mL). The combined organic layers were washed with brine (3×50 mL), dried over anhydrous sodium sulfate, concentrated and purified by column chromatography [SiO₂, Petroleum ether/Ethyl acetate=0/1 to 10/1] to afford the title compound (1.50 g, 50% yield) as yellow liquid; LCMS (ESI, M+1): m/z=353.1.

Step C. 6-chloro-5-((1S,2R)-2-methylcyclopropyl)-1-((2-(trimethylsilyl) ethoxy)methyl)-1H-indazol-4-yl trifluoromethanesulfonate: To a solution of 6-chloro-5-((1S,2R)-2-methylcyclopropyl)-1-((2-(trimethylsilyl) ethoxy)methyl)-1H-indazol-4-ol (4.10 g, 1.0 equiv) and DIEA (6.07 mL, 3.0 equiv) in DCM (40 mL) was added Tf₂O (3.83 mL, 2.0 equiv) at −70° C. The reaction was stirred at −70° C. for 0.5 hours. The mixture was diluted with water (40 mL) and extracted with DCM (3×30 mL). The combined organic layers were dried over anhydrous sodium sulfate, concentrated and purified by column chromatography [SiO₂, Petroleum ether/Ethyl acetate=1/0 to 5/1] to afford the title compound (4.50 g, 78% yield) as yellow liquid; ¹H NMR (400 MHZ, CHLOROFORM-d) δ−7.97 (d, J=6.8 Hz, 1H), 7.71 (dd, J=0.8, 9.2 Hz, 1H), 5.70 (d, J=5.6 Hz, 2H), 3.63-3.50 (m, 2H), 1.61-1.55 (m, 1H), 1.54-1.38 (m, 1H), 1.38-1.32 (m, 2H), 1.01-0.97 (m, 1H), 0.93-0.86 (m, 2H), 0.84-0.62 (m, 2H), −0.05 (d, J=4.0 Hz, 9H); LCMS (ESI, M+1): m/z=485.0.

Step D. 6-chloro-5-((1S,2R)-2-methylcyclopropyl)-4-(4,4,5,5-tetramethyl-1,3,2-dioxaborolan-2-yl)-1-((2-(trimethylsilyl) ethoxy)methyl)-1H-indazole: To a mixture of 6-chloro-5-((1S,2R)-2-methylcyclopropyl)-1-((2-(trimethylsilyl) ethoxy)methyl)-1H-indazol-4-yl trifluoromethanesulfonate (500 mg, 1.0 equiv), 4,4,4′,4′,5,5,5′,5′-octamethyl-2,2′-bi (1,3,2-dioxaborolane) (314 mg, 1.2 equiv) and KOAc (202 mg, 2.0 equiv) in MeCN (10 mL) was added P(Cy₃)-Pd-G3 (75.7 mg, 0.1 equiv). The reaction was degassed and purged with nitrogen 3 times. The reaction was stirred at 90° C. for 6 hours. The mixture was diluted with ethyl acetate (40 mL) and filtered, concentrated and purified with reversed phase flash chromatography [C18, 0.1% formic acid condition] to afford the title compound (850 mg, 44% yield) as yellow liquid; ¹H NMR (400 MHZ, CHLOROFORM-d) δ=8.12 (d, J=0.8 Hz, 1H), 7.65 (d, J=0.8 Hz, 1H), 5.67 (s, 2H), 3.55-3.45 (m, 2H), 1.47 (d, J=10.8 Hz, 12H), 1.32 (d, J=5.6 Hz, 3H), 1.27-1.26 (m, 1H), 0.92-0.78 (m, 5H), 0.01-−0.10 (m, 9H); LCMS (ESI, M+1): m/z=463.2.

Step E. 7-((R)-6-chloro-5-((1S,2R)-2-methylcyclopropyl)-1-((2-(trimethylsilyl) ethoxy)methyl)-1H-indazol-4-yl)-8-fluoro-2-((tetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-((tetrahydro-1H-pyrrolizin-7a(5H)-yl)methoxy)-4-(2,2,2-trifluoroethoxy)pyrido[4,3-d]pyrimidine (100 mg, 1.0 equiv), 6-chloro-5-((1S,2R)-2-methylcyclopropyl)-4-(4,4,5,5-tetramethyl-1,3,2-dioxaborolan-2-yl)-1-((2-(trimethylsilyl) ethoxy)methyl)-1H-indazole (121 mg, 1.1 equiv) and K₃PO₄ (1.5 M, 475 μL, 3.0 equiv) in toluene (2 mL) was added APhos-Pd-G3 (15.1 mg, 0.1 equiv). The reaction was degassed and purged with nitrogen 3 times. The reaction was stirred at 70° C. for 4 hours. The mixture was diluted with water (5 mL) and extracted with ethyl acetate (3×5 mL). The combined organic layers were dried over anhydrous sodium sulfate, concentrated and purified by column chromatography [Al₂O₃, Petroleum ether/Ethyl acetate=20/1 to 0/1] to afford the title compound (85.0 mg, 16% yield) as yellow solid; LCMS (ESI, M+1): m/z=721.6.

Step F. (R)-7-(7-((R)-6-chloro-5-((1S,2R)-2-methylcyclopropyl)-1-((2-(trimethylsilyl) ethoxy)methyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione: To a mixture of 7-((R)-6-chloro-5-((1S,2R)-2-methylcyclopropyl)-1-((2-(trimethylsilyl) ethoxy)methyl)-1H-indazol-4-yl)-8-fluoro-2-((tetrahydro-1H-pyrrolizin-7a(5H)-yl)methoxy)-4-(2,2,2-trifluoroethoxy)pyrido[4,3-d]pyrimidine (85 mg, 1.0 equiv), (R)-1,3,7-triazaspiro[4.5]decane-2,4-dione (39.9 mg, 2.0 equiv) and 4 Å molecular sieve (50 mg) in DMF (1 mL) was added DIEA (61.6 L, 3.0 equiv). The reaction was stirred at 40° C. for 12 hours. The mixture was filtered and purified with reversed phase flash chromatography [C18, 0.1% formic acid condition] to afford the title compound (50 mg, 53% yield) as yellow solid; LCMS (ESI, M+1): m/z=790.4.

Step G. (R)-7-(7-((R)-6-chloro-1-(hydroxymethyl)-5-((1S,2R)-2-methylcyclopropyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione: To a solution of (R)-7-(7-((R)-6-chloro-5-((1S,2R)-2-methylcyclopropyl)-1-((2-(trimethylsilyl) ethoxy)methyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione (40.0 mg, 1.0 equiv) in DCM (0.25 mL) was added TFA (0.5 mL, 133 equiv) at 0° C. The reaction was stirred at 0° C. for 0.5 hours. The mixture was adjusted pH to 8 with saturated NaHCO₃ aqueous solution (5 mL) and extracted with ethyl acetate (3×5 mL). The combined organic layers were dried over anhydrous sodium sulfate, concentrated to afford the title compound (35 mg, 99% yield) as yellow solid; LCMS (ESI, M+1): m/z=690.4.

Step H. (R)-7-(7-((R)-6-chloro-5-((1S,2R)-2-methylcyclopropyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione: To a solution of (R)-7-(7-((R)-6-chloro-1-(hydroxymethyl)-5-((1S,2R)-2-methylcyclopropyl)-1H-indazol-4-yl)-8-fluoro-2-((tetrahydro-1H-pyrrolizin-7a(5H)-yl)methoxy)pyrido[4,3-d]pyrimidin-4-yl)-1,3,7-triazaspiro[4.5]decane-2,4-dione (30 mg, 1.0 equiv) in THF (0.01 mL) was added NH₃·H₂O (5.46 g, 6.00 mL, 25% purity, 896 equiv). The reaction was stirred at 20° C. for 2 hours. The mixture was diluted with water (2 mL) and extracted with ethyl acetate (3×3 mL). The combined organic layers were dried over anhydrous sodium sulfate, concentrated and purified with prep-HPLC [Phenomenex C18 75×30 mm×3 μm; A: water (FA), B: ACN, B %: 12%-42% over 7 min] to afford the title compound (9.32 mg, 32% yield, HCOOH salt) as yellow solid; ¹H NMR (400 MHZ, METHANOL-d4) δ=9.34-9.12 (m, 1H), 7.99-7.59 (m, 2H), 4.69-4.57 (m, 2H), 4.50 (br d, J=8.0 Hz, 1H), 4.01-3.69 (m, 3H), 3.58-3.46 (m, 2H), 3.13 (br d, J=5.6 Hz, 2H), 2.36-1.90 (m, 12H), 1.81-1.65 (m, 1H), 1.10-0.72 (m, 3H), 0.64 (br d, J=4.4 Hz, 1H), 0.54-0.11 (m, 2H); LCMS (ESI, M+1): m/z=660.3.

### Example 888

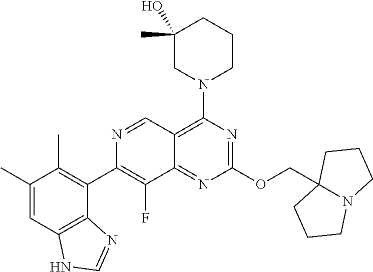 [SMILES: Cc1cc2[nH]cnc2c(-c2ncc3c(N4CCC[C@@](C)(O)C4)nc(OCC45CCCN4CCC5)nc3c2F)c1C]

### (3R)-1-(7-(5,6-dimethyl-1H-benzo[d]imidazol-4-yl)-8-fluoro-2-((tetrahydro-1H-pyrrolizin-7a(5H)-yl)methoxy)pyrido[4,3-d]pyrimidin-4-yl)-3-methylpiperidin-3-ol

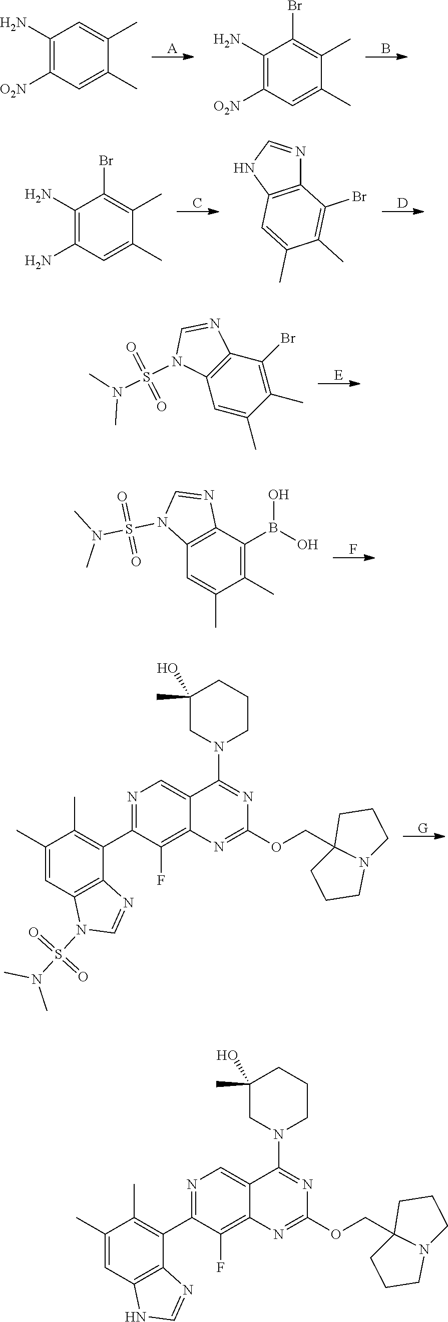

Step A. 2-bromo-3,4-dimethyl-6-nitroaniline: To a solution of 4,5-dimethyl-2-nitroaniline (10.0 g, 1.0 equiv) in acetonitrile (200 mL) was added NBS (11.8 g, 1.1 equiv). The reaction was stirred at 20° C. for 2 hours. The mixture was concentrated. The residue was diluted with water (100 mL) and extracted with EtOAc (4×100 mL). The combined organic layers were washed with brine (100 mL), dried over anhydrous sodium sulfate and concentrated to afford the title compound (15.0 g, crude) as yellow solid.

Step B. 3-bromo-4,5-dimethylbenzene-1,2-diamine: To a solution of 2-bromo-3,4-dimethyl-6-nitroaniline (15.0 g, 1.0 equiv) and NH₄Cl (13.1 g, 4.0 equiv) in ethyl alcohol (300 mL) and H₂O (150 mL) was added Zn powder (16.2 g, 4.1 equiv) slowly. The reaction was stirred at 60° C. for 30 minutes and 80° C. for 2 hours. The mixture was filtered. The filtrate was adjusted to pH=8 with saturated NaHCO₃ aqueous (100 mL) at 0° C. and extracted with EtOAc (4×80 mL). The combined organic layers were washed with brine (80 mL), dried over anhydrous sodium sulfate, concentrated and purified with column chromatography [SiO₂, Petroleum ether/Ethyl acetate=3/1 to 1/1] to afford the title compound (10.0 g, 76% yield) as yellow solid; ¹H NMR (400 MHz, CHLOROFORM-d) δ=6.50 (s, 1H), 3.56 (br s, 4H), 2.29 (s, 3H), 2.21 (s, 3H).

Step C. 4-bromo-5,6-dimethyl-1H-benzo[d]imidazole: A mixture of 3-bromo-4,5-dimethylbenzene-1,2-diamine (4.00 g, 1.0 equiv) in HCOOH (40 mL) was stirred at 100° C. for 2 hours. The mixture was concentrated. The mixture was adjusted to pH=8 with saturated Na₂CO₃ aqueous (100 mL) and extracted with EtOAc (4×80 mL). The combined organic layers were washed with brine (100 mL), dried over anhydrous sodium sulfate, concentrated and purified with column chromatography [SiO₂, Petroleum ether/Ethyl acetate=1/1 to 0/1] to afford the title compound (3.20 g, 73% yield) as yellow solid; LCMS (ESI, M+1, M+3): m/z=224.9, 226.9.

Step D. 4-bromo-N,N,5,6-tetramethyl-1H-benzo[d]imidazole-1-sulfonamide: To a solution of 4-bromo-5,6-dimethyl-1H-benzo[d]imidazole (2.50 g, 1.0 equiv) in DMAc (30 mL) was added NaH (1.33 g, 60% purity, 3.0 equiv) slowly at 0° C. for 0.5 hours. The reaction was stirred at 0° C. for 0.5 hours. Then dimethylsulfamoyl chloride (2.39 g, 1.5 equiv) was added into the mixture at 0° C. The reaction was stirred at 0° C. for 0.5 hours. The mixture was diluted with water (30 mL) at 0° C. and extracted with EtOAc (4×30 mL). The combined organic layers were washed with brine (30 mL), dried over anhydrous sodium sulfate, concentrated and purified with reversed phase flash chromatography [C18, 0.1% formic acid condition] to afford the title compound (3.50 g, 93% yield) as yellow solid; LCMS (ESI, M+1, M+3): m/z=331.9, 333.9.

Step E. (1-(N,N-dimethylsulfamoyl)-5,6-dimethyl-1H-benzo[d]imidazol-4-yl) boronic acid: To a mixture of 4,4,4′,4′,5,5,5′,5′-octamethyl-2,2′-bi (1,3,2-dioxaborolane) (4.59 g, 3.0 equiv), 4-bromo-N,N,5,6-tetramethyl-1H-benzo[d]imidazole-1-sulfonamide (2.00 g, 1.0 equiv) and KOAc (1.18 g, 2.0 equiv) in toluene (30 mL) was added P(Cy₃) Pd G3 (391 mg, 0.10 equiv). The reaction was degassed and purged with nitrogen 3 times. The reaction was stirred at 100° C. for 12 hours. The mixture was diluted with water (30 mL) and extracted with EtOAc (4×30 mL). The combined organic layers were dried over anhydrous sodium sulfate, concentrated and purified with reversed phase flash chromatography [C18, 0.1% NH₃·H₂O condition] to afford the title compound (500 mg, 25% yield) as yellow solid; LCMS (ESI, M+1): m/z=298.0.

Step F. 4-(8-fluoro-4-((R)-3-hydroxy-3-methylpiperidin-1-yl)-2-((tetrahydro-1H-pyrrolizin-7a(5H)-yl)methoxy)pyrido[4,3-d]pyrimidin-7-yl)-N,N,5,6-tetramethyl-1H-benzo[d]imidazole-1-sulfonamide: To a mixture of (R)-1-(7-chloro-8-fluoro-2-((tetrahydro-1H-pyrrolizin-7a(5H)-yl)methoxy)pyrido[4,3-d]pyrimidin-4-yl)-3-methylpiperidin-3-ol (100 mg, 1.0 equiv) and (1-(N,N-dimethylsulfamoyl)-5,6-dimethyl-1H-benzo[d]imidazol-4-yl) boronic acid (102 mg, 1.5 equiv) in toluene (2 mL) and H₂O (459 μL) were added K₃PO₄ (146 mg, 3.0 equiv) and Aphos-Pd-G3 (14.6 mg, 0.10 equiv). The reaction was degassed and purged with nitrogen 3 times. The reaction was stirred at 60° C. for 12 hours. The mixture was diluted with water (5 mL) and extracted with EtOAc (4×5 mL). The organic phase was dried over anhydrous sodium sulfate, concentrated and purified with reversed phase flash chromatography [C18, 0.1% formic acid condition] to afford the title compound (100 mg, 63% yield) as yellow solid; LCMS (ESI, M+1): m/z=653.2.

Step G. (3R)-1-(7-(5,6-dimethyl-1H-benzo[d]imidazol-4-yl)-8-fluoro-2-((tetrahydro-1H-pyrrolizin-7a(5H)-yl)methoxy)pyrido[4,3-d]pyrimidin-4-yl)-3-methylpiperidin-3-ol: To a solution of 4-(8-fluoro-4-((R)-3-hydroxy-3-methylpiperidin-1-yl)-2-((tetrahydro-1H-pyrrolizin-7a(5H)-yl)methoxy)pyrido[4,3-d]pyrimidin-7-yl)-N,N,5,6-tetramethyl-1H-benzo[d]imidazole-1-sulfonamide (130 mg, 1.0 equiv) in EtOH (0.5 mL) was added HCl (12 M, 2 mL). The reaction was stirred at 50° C. for 2 hours. The mixture was quenched with saturated Na₂CO₃ aqueous (10 mL) at 0° C. and extracted with EtOAc (4×5 mL). The combined organic layers was dried over anhydrous sodium sulfate, concentrated and purified with prep-TLC [SiO₂, Dichloromethane:Methanol=10/1] and prep-HPLC [Phenomenex luna C18 150×25 mm×10 μm; A: water (FA), B: ACN; B %: 0%-30%, over 10 min] twice to afford the title compound (6.57 mg, 5.5% yield, 0.73 HCOOH) as white solid; ¹H NMR (400 MHZ, METHANOL-d₄) δ=9.30 (s, 1H), 8.05 (s, 1H), 7.57 (s, 1H), 4.60 (br s, 3H), 4.34 (br d, J=13.2 Hz, 1H), 3.67-3.55 (m, 3H), 3.50-3.38 (m, 1H), 3.24-3.12 (m, 2H), 2.49 (s, 3H), 2.34-2.24 (m, 2H), 2.23-2.20 (m, 3H), 2.20-2.13 (m, 3H), 2.13-1.99 (m, 4H), 1.93-1.84 (m, 1H), 1.84-1.74 (m, 2H), 1.30 (s, 3H); LCMS (ESI, M+1): m/z=546.4.

### Example 889

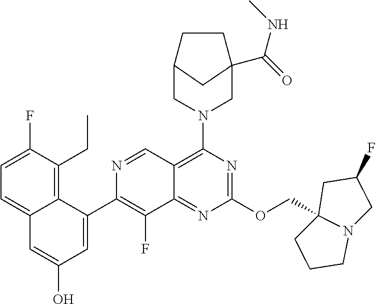 [SMILES: CCc1c(F)ccc2cc(O)cc(-c3ncc4c(N5CC6CCC(C(=O)NC)(C6)C5)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.1]octane-1-carboxamide

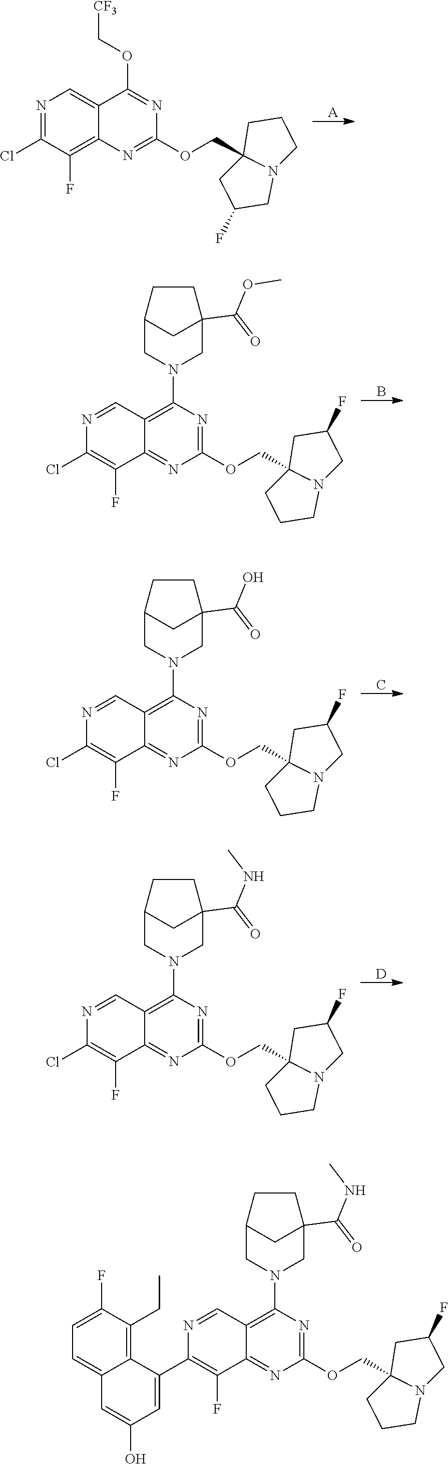

Step A. methyl 3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate: To a mixture of methyl 3-azabicyclo[3.2.1]octane-1-carboxylate (609 mg, 1.3 equiv, HCl) and DIEA (1.60 mL, 4.0 equiv) in DMF (4 mL) was added 4 Å molecular sieve (50 mg). The reaction was stirred at 20° C. for 10 minutes. 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (1 g, 1.0 equiv) was added into the mixture. The reaction was stirred at 40° C. for 12 hours. The mixture was filtered and purified with reversed phase (0.1% FA condition) to afford the title compound (900 mg, 76% yield) as yellow solid; LCMS (ESI, M+1): m/z=508.2.

Step B. 3-(7-chloro-8-fluoro-2-(((2R,7aS)-2-fluorohexahydro-1H-pyrrolizin-7a-yl)methoxy)pyrido[4,3-d]pyrimidin-4-yl)-3-azabicyclo[3.2.1]octane-1-carboxylic acid: To a solution of methyl 3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylate (400 mg, 1.0 equiv) in THF (5 mL) and H₂O (1 mL) was added NaOH (157 mg, 5.0 equiv) at 0° C. The reaction was stirred at 20° C. for 1 hour. The mixture was adjusted pH to 6 by 1 N HCl aqueous at 0° C. and purified with reversed phase (0.1% FA condition) to afford the title compound (235 mg, 56% yield) as yellow solid; LCMS (ESI, M+1): m/z=494.1.

Step C. 3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.1]octane-1-carboxamide: To a solution of 3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octane-1-carboxylic acid (135 mg, 1.0 equiv) in DMF (2 mL) were added TEA (442 mg, 16.0 equiv) and T3P (1.64 g, 50% purity, 9.4 equiv) dropwise at 0° C. The reaction was stirred at 0° C. for 0.1 hours. methanamine (284 mg, 10.0 equiv, HCl) was added into above mixture. The reaction was stirred at 20° C. for 1 hour. The mixture was added saturated NaHCO₃ aqueous (16 mL) at 0° C. and extracted with EtOAc (8×4 mL). The combined organic layers were dried over anhydrous sodium sulfate and concentrated to afford the title compound (220 mg, crude) as yellow solid; LCMS (ESI, M+1): m/z=507.2.

Step D. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.1]octane-1-carboxamide: To a solution of 3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-N-methyl-3-azabicyclo[3.2.1]octane-1-carboxamide (200 mg, 1.0 equiv) and 5-ethyl-6-fluoro-4-(4,4,5,5-tetramethyl-1,3,2-dioxaborolan-2-yl)naphthalen-2-ol (162 mg, 1.3 equiv) in methoxycyclopentane (4 mL) were added CataCXium A Pd G3 and K₃PO₄ (1.5 M, 789 μL, 3.0 equiv). The reaction was degassed and purged with nitrogen 3 times. The reaction was stirred at 80° C. for 4 hours. The mixture was diluted with water (3 mL) and extracted with EtOAc (4×4 mL). The combined organic layers were dried over anhydrous sodium sulfate, concentrated and purified with reversed-phase (0.1% FA condition) and prep-HPLC (column: Waters Xbridge 150×25 mm×5 um; mobile phase: [water (NH₄HCO₃)-ACN]; B %: 43%-73%, 8 minutes) to afford the title compound (95 mg, 36% yield) as white solid; 1H NMR (400 MHZ, METHANOL-d4) δ=9.08 (d, J=1.6 Hz, 1H), 7.67 (dd, J=6.0, 9.2 Hz, 1H), 7.32-7.20 (m, 2H), 7.06 (t, J=2.4 Hz, 1H), 5.43-5.19 (m, 1H), 5.06-4.90 (m, 2H), 4.58 (br s, 1H), 4.38-4.21 (m, 2H), 3.73-3.50 (m, 2H), 3.26-3.17 (m, 2H), 3.01 (dt, J=5.6, 9.2 Hz, 1H), 2.77 (d, J=2.8 Hz, 3H), 2.59-2.41 (m, 2H), 2.40-2.10 (m, 4H), 2.09-1.94 (m, 4H), 1.88 (br s, 4H), 1.70-1.51 (m, 1H), 0.80 (br t, J=7.2 Hz, 3H); LCMS (ESI, M+1): m/z=661.4.

### Example 890

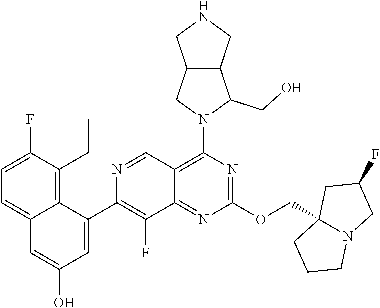 [SMILES: CCc1c(F)ccc2cc(O)cc(-c3ncc4c(N5CC6CNCC6C5CO)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-(hydroxymethyl)hexahydropyrrolo[3,4-c]pyrrol-2 (1H)-yl)pyrido[4,3-d]pyrimidin-7-yl)naphthalen-2-ol

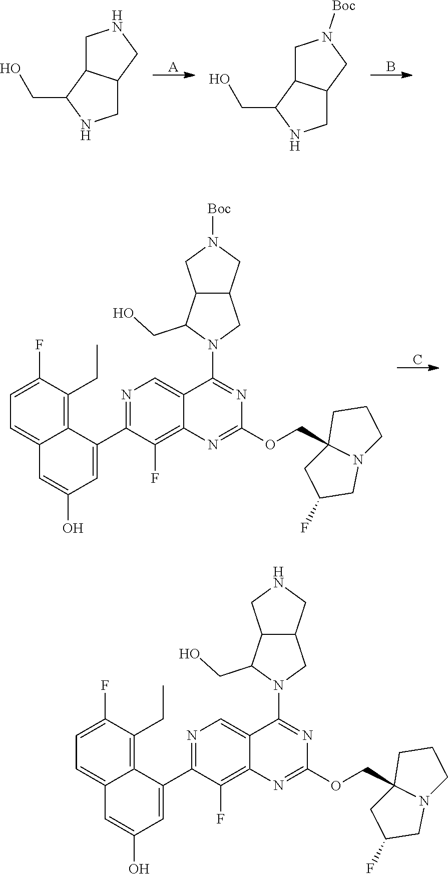

Step A. tert-butyl 4-(hydroxymethyl)hexahydropyrrolo[3,4-c]pyrrole-2 (1H)-carboxylate: To a solution of (octahydropyrrolo[3,4-c]pyrrol-1-yl)methanol (200 mg, 1.0 equiv, 2 HCl salt) and TEA (564 mg, 6.0 equiv) in DCM (2 mL) and MeOH (2 mL) were added Boc₂O (223 mg, 1.1 equiv). The reaction was stirred at 25° C. for 12 hours. The mixture was concentrated to afford the title compound (220 mg, crude) as a yellow solid.

Step B. tert-butyl 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-4-(hydroxymethyl)hexahydropyrrolo[3,4-c]pyrrole-2 (1H)-carboxylate: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (289 mg, 1.0 equiv) and tert-butyl 4-(hydroxymethyl)hexahydropyrrolo[3,4-c]pyrrole-2 (1H)-carboxylate (220 mg, 2.0 equiv) in DMF (2 mL) were added DIEA (293 mg, 5.0 equiv) and 4 Å molecular sieve (200 mg). The reaction was stirred at 40° C. for 12 hours. The mixture was concentrated and purified by prep-HPLC (column: Waters Xbridge 150*25 mm*5 um; mobile phase: [water (ammonia hydroxide v/v)-ACN]; B %: 47%-77%, 9 min) to afford the title compound (18.0 mg, 45% yield) as a white solid; LCMS (ESI, M+1): m/z=735.4.

Step C. 5-ethyl-6-fluoro-4-(8-fluoro-2-(((2R,7aS)-2-fluorohexahydro-1H-pyrrolizin-7a-yl)methoxy)-4-(1-(hydroxymethyl)hexahydropyrrolo[3,4-c]pyrrol-2 (1H)-yl)pyrido[4,3-d]pyrimidin-7-yl)naphthalen-2-ol: To a solution of tert-butyl 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-4-(hydroxymethyl)hexahydropyrrolo[3,4-c]pyrrole-2 (1H)-carboxylate (15.0 mg, 1.0 equiv) in DCM (0.1 mL) was added HCl·dioxane (4 M, 75.00 uL). The reaction was stirred at 25° C. for 0.5 hours. The mixture was concentrated and purified by prep-HPLC (column: Phenomenex C18 75*30 mm*3 um; mobile phase: [water (FA)-ACN]; B %: 2%-32%, 7 min) to afford the title compound (2.70 mg, 21% yield) as off-white solid. ¹HNMR (400 MHZ, METHANOL-d4) δ=9.34-9.19 (m, 1H), 8.48 (br d, J=1.2 Hz, 1H), 7.69 (dd, J=6.0, 9.2 Hz, 1H), 7.39-7.20 (m, 2H), 7.08-6.95 (m, 1H), 5.52-5.29 (m, 2H), 5.14-5.00 (m, 3H), 4.47-4.28 (m, 2H), 4.25-4.09 (m, 1H), 4.08-3.97 (m, 1H), 3.88 (br dd, J=1.6, 13.2 Hz, 1H), 3.74-3.52 (m, 3H), 3.49-3.38 (m, 2H), 3.22-3.05 (m, 2H), 2.62-2.40 (m, 1H), 2.39-2.28 (m, 1H), 2.25-2.06 (m, 4H), 2.01-1.87 (m, 1H), 1.18 (t, J=7.2 Hz, 2H), 0.85-0.75 (m, 3H): LCMS (ESI, M+1): m/z=635.4.

### Example 891

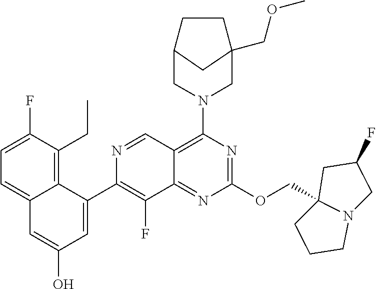 [SMILES: CCc1c(F)ccc2cc(O)cc(-c3ncc4c(N5CC6CCC(COC)(C6)C5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(1-(methoxymethyl)-3-azabicyclo[3.2.1]octan-3-yl)pyrido[4,3-d]pyrimidin-7-yl)naphthalen-2-ol

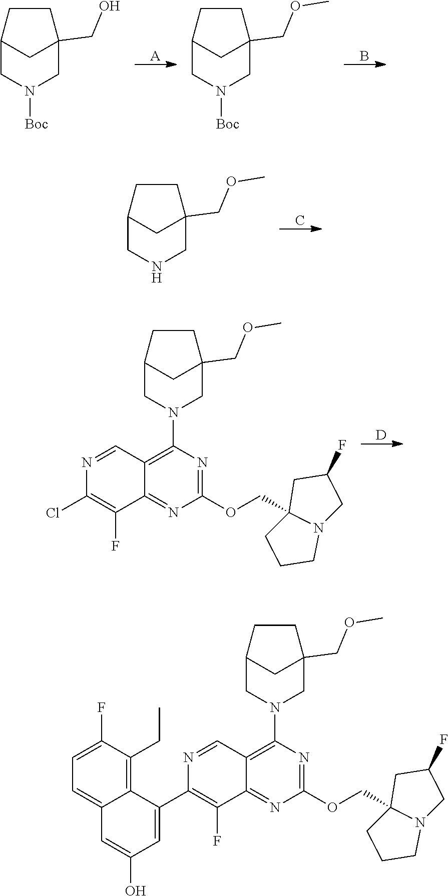

Step A. tert-butyl 1-(methoxymethyl)-3-azabicyclo[3.2.1]octane-3-carboxylate: To a solution of tert-butyl 1-(hydroxymethyl)-3-azabicyclo[3.2.1]octane-3-carboxylate (100 mg, 1.0 equiv) in DMAc (2 mL) was added NaH (49.7 mg, 60% purity, 3.0 equiv) portionwise at 0° C. The reaction was stirred at 0° C. for 0.5 hours. MeI (118 mg, 2.0 equiv) was added at 0° C. The mixture was stirred at 25° C. for 3 hours. The mixture was quenched with water (5 mL) at 0° C. and extracted with ethyl acetate (10 mL). The organic layer was dried over sodium sulfate and concentrated to afford the title compound (105 mg, crude) as yellow oil.

Step B. 1-(methoxymethyl)-3-azabicyclo[3.2.1]octane: To a solution of tert-butyl 1-(methoxymethyl)-3-azabicyclo[3.2.1]octane-3-carboxylate (105 mg, 1.0 equiv) in MeCN (2 mL) was added HCl/dioxane (4 M, 4 mL, 39 equiv) at 0° C. The reaction was stirred at 0° C. for 0.5 hours. The mixture was concentrated to afford the title compound (63.0 mg, crude, HCl) as white solid.

Step C. 7-chloro-8-fluoro-2-(((2R,7aS)-2-fluorotetrahydro-1H-pyrrolizin-7a(5H)-yl)methoxy)-4-(1-(methoxymethyl)-3-azabicyclo[3.2.1]octan-3-yl)pyrido[4,3-d]pyrimidine: To a solution of 1-(methoxymethyl)-3-azabicyclo[3.2.1]octane (52.4 mg, 1.5 equiv, HCl) and 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (80.0 mg, 1.0 equiv) in DMF (1 mL) was added DIEA (118 mg, 5.0 equiv) at 25° C. The reaction was stirred at 40° C. for 14 hours. The mixture was diluted with water (5 mL) and extracted with ethyl acetate (10 mL). The organic layer was dried over sodium sulfate and concentrated to afford the title compound (90.0 mg, crude) as yellow oil.

Step D. 5-ethyl-6-fluoro-4-(8-fluoro-2-(((2R,7aS)-2-fluorotetrahydro-1H-pyrrolizin-7a(5H)-yl)methoxy)-4-(1-(methoxymethyl)-3-azabicyclo[3.2.1]octan-3-yl)pyrido[4,3-d]pyrimidin-7-yl)naphthalen-2-ol: A mixture of 7-chloro-8-fluoro-2-(((2R,7aS)-2-fluorotetrahydro-1H-pyrrolizin-7a(5H)-yl)methoxy)-4-(1-(methoxymethyl)-3-azabicyclo[3.2.1]octan-3-yl)pyrido[4,3-d]pyrimidine (90.0 mg, 1.0 equiv), 5-ethyl-6-fluoro-4-(4,4,5,5-tetramethyl-1,3,2-dioxaborolan-2-yl)naphthalen-2-ol (86.4 mg, 1.5 equiv), cataCXium A Pd G3 (26.5 mg, 0.2 equiv) and K₃PO₄ (1.5 M, 364 μL, 3.0 equiv) in methoxycyclopentane (5 mL) was degassed and purged with N₂ 3 times. The reaction was stirred at 80° C. for 3 hours under N₂ atmosphere. The mixture was diluted with water (20 mL) and extracted with ethyl acetate (30 mL). The organic layer was dried over sodium sulfate, concentrated. The residue was purified by column chromatography (SiO₂, Dichloromethane:Methanol=100/1 to 10/1), and further purified by prep-HPLC (column: Phenomenex luna C18 150×25 mm×10 um; mobile phase: A: water (FA), B: ACN; B %: 28% to 48% over 9 min). to afford the title compound (27.6 mg, 23% yield) as white solid; ¹H NMR (400 MHz, methanol-d4) δ=9.09 (d, J=4.4 Hz, 1H), 7.68 (dd, J=6.0, 9.2 Hz, 1H), 7.31 (d, J=2.8 Hz, 1H), 7.25 (t, J=9.6 Hz, 1H), 7.07 (d, J=2.0 Hz, 1H), 5.55-5.24 (m, 1H), 4.75-4.71 (m, 1H), 4.50-4.34 (m, 2H), 3.66-3.38 (m, 8H), 3.36 (d, J=4.8 Hz, 3H), 3.21-3.12 (m, 1H), 2.54-2.45 (m, 2H), 2.44-2.29 (m, 2H), 2.28-2.17 (m, 2H), 2.16-2.05 (m, 2H), 2.04-1.93 (m, 1H), 1.89-1.73 (m, 2H), 1.69-1.46 (m, 4H), 0.86-0.74 (m, 3H); LCMS (ESI, M+1, M/2+1): m/z=648.3, 324.7.

### Example 892

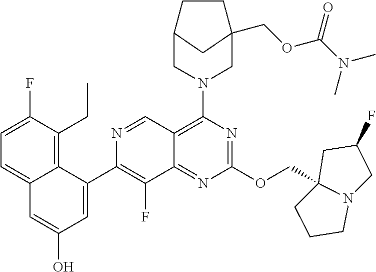 [SMILES: CCc1c(F)ccc2cc(O)cc(-c3ncc4c(N5CC6CCC(COC(=O)N(C)C)(C6)C5)nc(OC[C@@]56CCCN5C[C@H](F)C6)nc4c3F)c12]

### (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)methyl dimethylcarbamate

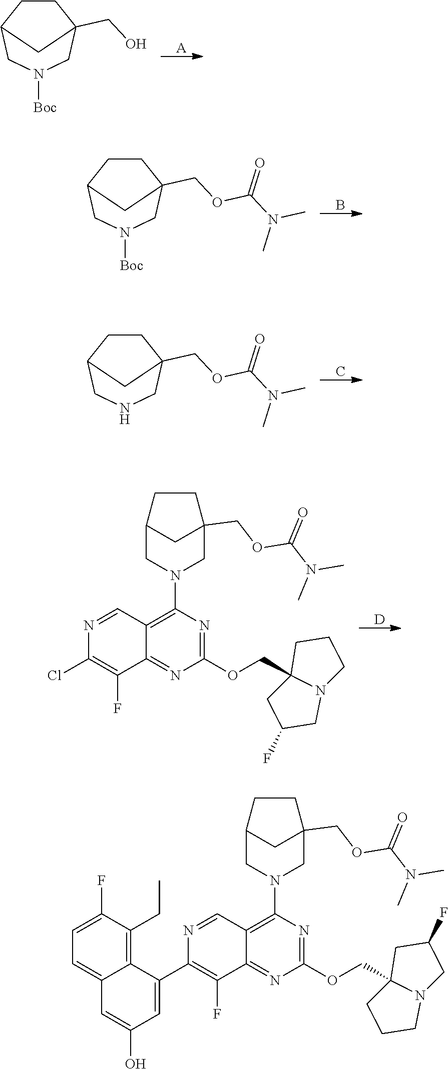

Step A. tert-butyl 1-(((dimethylcarbamoyl)oxy)methyl)-3-azabicyclo[3.2.1]octane-3-carboxylate: To a solution of tert-butyl 1-(hydroxymethyl)-3-azabicyclo[3.2.1]octane-3-carboxylate (100 mg, 1.0 equiv) in DMAc (2.00 mL) was added NaH (49.7 mg, 60% purity, 3.0 equiv) portionwise at 0° C. The mixture was stirred at 0° C. for 0.5 hours. N,N-dimethylcarbamoyl chloride (89.1 mg, 2.0 equiv) was added at 0° C. The reaction was stirred at 25° C. for 3 hours. The mixture was quenched by water (5 mL) at 0° C. and extracted with ethyl acetate (10 mL). The organic layer was dried over sodium sulfate, concentrated to afford the title compound (150 mg, crude) as yellow solid.

Step B. (3-azabicyclo[3.2.1]octan-1-yl)methyl dimethylcarbamate: To a solution of tert-butyl 1-(dimethylcarbamoyloxymethyl)-3-azabicyclo[3.2.1]octane-3-carboxylate (100 mg, 1.0 equiv) in ACN (1.5 mL) was added HCl/dioxane (4 M, 80.0 uL, 1.0 equiv) at 0° C. The reaction was stirred at 25° C. for 0.5 hours. The mixture was concentrated to afford the title compound (77.0 mg, crude) as a yellow solid.

Step C. (3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)methyl dimethylcarbamate: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (90 mg, 1.0 equiv) and 3-azabicyclo[3.2.1]octan-1-ylmethyl N,N-dimethylcarbamate (76.5 mg, 1.5 equiv, HCl) in DMF (1.00 mL) was added DIEA (79.5 mg, 3.0 equiv). The reaction was stirred at 40° C. for 14 hours. The mixture was diluted with water (1 mL) and extracted with ethyl acetate (3 mL). The organic layer was dried over sodium sulfate, concentrated to afford the title compound (130 mg, crude) as yellow oil. LCMS (ESI, M+1): m/z=551.1.

Step D. (3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)methyl dimethylcarbamate: To a solution of (3-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)methyl dimethylcarbamate (130 mg, 61% purity, 1.0 equiv) and 5-ethyl-6-fluoro-4-(4,4,5,5-tetramethyl-1,3,2-dioxaborolan-2-yl)naphthalen-2-ol (68.25 mg, 1.5 equiv) in methoxycyclopentane (3.00 mL) was added cataCXium® A Pd G3 (21.0 mg, 0.2 equiv) and aqueous K₃PO₄ (1.5 M, 288 μL, 3.0 equiv). The mixture was degassed and purged with N₂ for 3 times. The mixture was stirred at 80° C. for 3 hours under N₂ atmosphere. The mixture was diluted with water (3 mL) and extracted with ethyl acetate (4 mL). The organic layer was dried over sodium sulfate, concentrated and purified by prep-HPLC [column: Phenomenex lun 150×25 mm×10 μm; A: water (FA), B: ACN; B %: 24%-54% over 10 min] to afford the title compound (32.1 mg, 31% yield, HCOOH) as a white solid. ¹H NMR (400 MHZ, METHANOL-d₄) δ=9.10 (d, J=3.6 Hz, 1H), 7.67 (dd, J=5.6, 9.2 Hz, 1H), 7.30 (d, J=2.8 Hz, 1H), 7.25 (t, J=9.2 Hz, 1H), 7.08-7.02 (m, 1H), 5.47-5.20 (m, 1H), 4.79-4.66 (m, 2H), 4.41-4.25 (m, 2H), 4.22-4.05 (m, 2H), 3.63 (td, J=5.6, 11.6 Hz, 1H), 3.53 (td, J=7.8, 12.3 Hz, 1H), 3.46-3.32 (m, 2H), 3.30-3.23 (m, 1H), 3.08 (dt, J=5.8, 9.6 Hz, 1H), 2.93 (br d, J=12.4 Hz, 6H), 2.55-2.44 (m, 2H), 2.43-2.31 (m, 1H), 2.31-2.24 (m, 1H), 2.23-2.12 (m, 2H), 2.09-2.00 (m, 2H), 1.98-1.79 (m, 3H), 1.74-1.64 (m, 2H), 1.63-1.52 (m, 2H), 0.84-0.74 (m, 3H). LCMS (ESI, M+1): m/z=705.3.

### Example 893

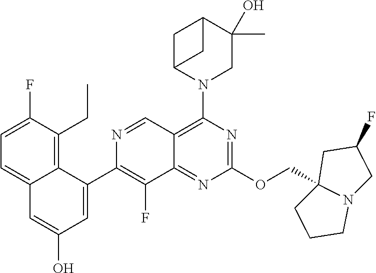 [SMILES: CCc1c(F)ccc2cc(O)cc(-c3ncc4c(N5CC(C)(O)C6CC5C6)nc(OC[C@@]56CCCN5C[C@H](F)C6)nc4c3F)c12]

### 2-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-4-methyl-2-azabicyclo[3.1.1]heptan-4-ol

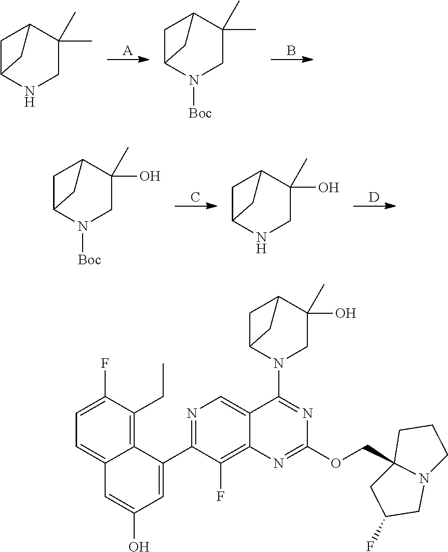

Step A. tert-butyl 4-oxo-2-azabicyclo[3.1.1]heptane-2-carboxylate: To a solution of 2-azabicyclo[3.1.1]heptan-4-one (100 mg, 1.0 equiv) and TEA (376 uL, 3 equiv) in DCM (1.5 mL) were added N,N-dimethylpyridin-4-amine (21.3 mg, 0.2 equiv) and Boc₂O (393 mg, 2 equiv). The reaction was stirred at 25° C. for 1 hour. The mixture was diluted with H₂O (5 mL) and extracted with DCM (10 mL×3). The combined organic layers were washed with brine (10 mL), dried over anhydrous sodium sulfate, filtered and concentrated to afford the title compound (160 mg, 84% yield) as yellow solid; ¹H NMR (400 MHZ, DMSO-d₆) δ=4.67-4.63 (m, 1H), 4.12-4.08 (m, 2H), 2.90 (q, J=5.6 Hz, 1H), 2.68-2.61 (m, 2H), 1.84 (br s, 2H), 1.41 (s, 9H).

Step B. tert-butyl 4-hydroxy-4-methyl-2-azabicyclo[3.1.1]heptane-2-carboxylate: To a solution of tert-butyl 4-oxo-2-azabicyclo[3.1.1]heptane-2-carboxylate (80 mg, 1 equiv) in THF (1 mL) was added MeMgBr (1 M, 568 μL, 1.5 equiv) at 0° C. The reaction was stirred at 25° C. for 2 hours. The mixture was quenched with saturated NH₄Cl solution (5 mL) and extracted with DCM (10 mL×3). The combined organic layers were washed with brine (10 mL), dried over anhydrous sodium sulfate, concentrated and purified with flash silica gel chromatography (ISCO®; 4 g SepaFlash® Silica Flash Column, Eluent of 0˜50% Ethylacetate/Petroleum ethergradient@20 mL/min) to afford the title compound (47 mg, crude) as light yellow oil.

Step C. 4-methyl-2-azabicyclo[3.1.1]heptan-4-ol: To a solution of tert-butyl 4-hydroxy-4-methyl-2-azabicyclo[3.1.1]heptane-2-carboxylate (40 mg, 1 equiv) in DCM (1 mL) was added TFA (0.5 mL, 38 equiv). The reaction was stirred at 25° C. for 2 hours. The mixture was concentrated under reduced pressure to afford the title compound (23 mg, crude) as white oil.

Step D. 2-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)pyrido[4,3-d]pyrimidin-4-yl)-4-methyl-2-azabicyclo[3.1.1]heptan-4-ol: To a mixture of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30 mg, 1 equiv) and 4-methyl-2-azabicyclo[3.1.1]heptan-4-ol (6.44 mg, 1 equiv) in DMF (1 mL) was added K₃PO₄ (21.5 mg, 2 equiv). The reaction was stirred at 60° C. for 2 hours. The mixture was filtered and purified by prep-HPLC (column: Boston Green ODS 150*30 mm*5 um; mobile phase: [water (FA)-ACN]; B %: 20%-50%, 6 min) to afford the title compound (4.25 mg, 13% yield, 0.39HCOOH) as white solid. ¹H NMR (400 MHZ, CD₃OD) δ=8.94 (br s, 1H), 7.71-7.65 (m, 1H), 7.31 (d, J=2.4 Hz, 1H), 7.29-7.21 (m, 1H), 7.09-7.02 (m, 1H), 5.45-5.26 (m, 1H), 4.48-4.13 (m, 5H), 3.50-3.37 (m, 1H), 3.11-3.04 (m, 1H), 2.67-1.81 (m, 15H), 1.48-1.27 (m, 3H), 0.86-0.75 (m, 3H); LCMS (ESI, M+1): m/z=620.3.

### Example 894

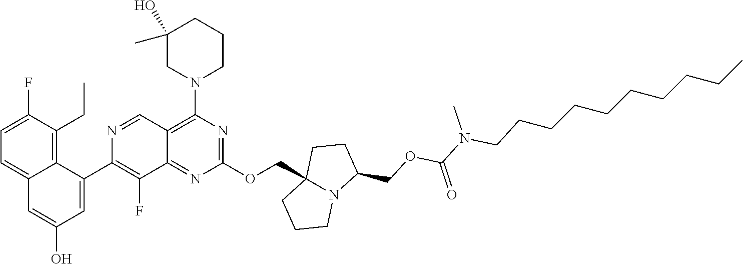 [SMILES: CCCCCCCCCCN(C)C(=O)OC[C@@H]1CC[C@@]2(COc3nc(N4CCC[C@@](C)(O)C4)c4cnc(-c5cc(O)cc6ccc(F)c(CC)c56)c(F)c4n3)CCCN12]

### ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl decyl(methyl)carbamate

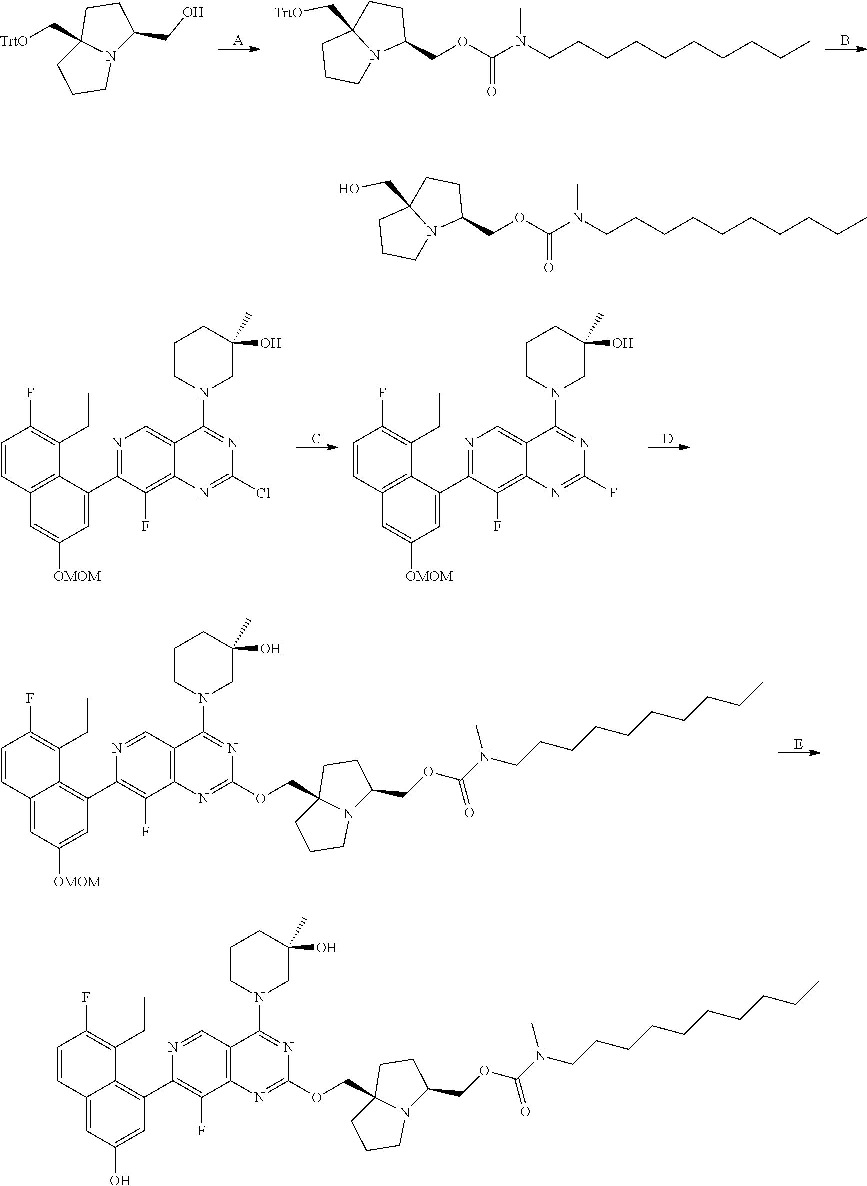

Step A. ((3S,7aR)-7a-((trityloxy)methyl) hexahydro-1H-pyrrolizin-3-yl)methyl decyl(methyl)carbamate: To a solution of ((3S,7aR)-7a-((trityloxy)methyl) hexahydro-1H-pyrrolizin-3-yl)methanol (200 mg, 1.0 equiv) and TEA (245 mg, 5.0 equiv) in THF (10 mL) was added (4-nitrophenyl) carbonochloridate (195 mg, 2.0 equiv) dropwise at 40° C. After stirring at 40° C. for 3 hours, a solution of N-methyldecan-1-amine; hydrochloride (110 mg, 1.1 equiv) in DMF (0.5 mL) was added to the mixture. The reaction was stirred at 80° C. for 9 hours. The mixture was diluted with H₂O (30 mL) and extracted with ethyl acetate (2× 30 mL). Combined organic layers were washed with brine (10 mL), dried over anhydrous sodium sulfate, concentrated, and purified with prep-TLC (SiO₂, Petroleum ether/Ethyl acetate=5/1) to afford the title compound (150 mg, 51% yield) as yellow solid; LCMS (ESI, M+1): m/z=611.6.

Step B. ((3S,7aR)-7a-(hydroxymethyl) hexahydro-1H-pyrrolizin-3-yl)methyl decyl(methyl)carbamate: To a solution of ((3S,7aR)-7a-((trityloxy)methyl) hexahydro-1H-pyrrolizin-3-yl)methyl decyl(methyl)carbamate (196 mg, 1 equiv) in DCM (5 mL) was added TFA (4.62 g, 126 equiv). The reaction was stirred at 25° C. for 18 hours. The mixture was concentrated under reduced pressure to afford the title compound (189 mg, crude) as yellow oil which used for the next step without further purification; LCMS (ESI, M+1): m/z=369.3.

Step C. (R)-1-(7-(8-ethyl-7-fluoro-3-(methoxymethoxy)naphthalen-1-yl)-2,8-difluoropyrido[4,3-d]pyrimidin-4-yl)-3-methylpiperidin-3-ol: To a solution of (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (100 mg, 1.0 equiv) in DMSO (2 mL) was added KF (220 mg, 3.79 mmol, 20 equiv). The reaction was stirred at 120° C. for 0.5 hours. The mixture was diluted with EtOAc (20 mL). The combined organic layers were washed with brine (20 mL×3), dried over anhydrous sodium sulfate, concentrated, and purified with flash silica gel chromatography (ISCO®; 4 g SepaFlash® Silica Flash Column, Eluent of 0˜10% Dichloromethane/Methanol @15 ml/min) to afford the title compound (92 mg, 74% yield) as yellow oil; (ESI, M+1): m/z=513.1

Step D. ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl decyl(methyl)carbamate: To a solution of (R)-1-(7-(8-ethyl-7-fluoro-3-(methoxymethoxy)naphthalen-1-yl)-2,8-difluoropyrido[4,3-d]pyrimidin-4-yl)-3-methylpiperidin-3-ol (92.0 mg, 1 equiv) and ((3S,7aR)-7a-(hydroxymethyl) hexahydro-1H-pyrrolizin-3-yl)methyl decyl(methyl)carbamate (86.0 mg, 1.0 equiv) in THF (1 mL) was added t-BuOK (1 M, 420 μL, 3 equiv). The reaction was stirred at 0° C. for 1 hour. The mixture was quenched with addition H₂O (10 mL) and extracted with DCM (3×10 mL). The combined organic layers were dried over anhydrous sodium sulfate, concentrated and purified with flash silica gel chromatography ((ISCO®: 4 g SepaFlash® Silica Flash Column, Eluent of 0˜10% Dichloromethane/Methanol @15 ml/min) to afford the title compound (69 mg, 43% yield) as a yellow oil; LCMS (ESI, M+1): m/z=861.3.

Step E. ((3S,7aR)-7a-(((7-(8-ethyl-7-fluoro-3-hydroxynaphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl decyl(methyl)carbamate: To a mixture of ((3S,7aR)-7a-(((7-(8-ethyl-7-fluoro-3-(methoxymethoxy)naphthalen-1-yl)-8-fluoro-4-((R)-3-hydroxy-3-methylpiperidin-1-yl)pyrido[4,3-d]pyrimidin-2-yl)oxy)methyl) hexahydro-1H-pyrrolizin-3-yl)methyl decyl(methyl)carbamate (69 mg, 1.0 equiv) in dioxane (1 mL) was added HCl·dioxane (4 M, 0.5 mL). The mixture was stirred at 25° C. for 2 hours. The mixture was concentrated and purified by prep-HPLC (column: O-Welch C18 150*30 mm*5 um; mobile phase: [water (FA)-ACN]; B %: 25%-65%, 10 min) to afford the title compound (4.07 mg, 6.1% yield, 0.23 HCOOH) as a white solid; 1H NMR (400 MHZ, METHANOL-d4) δ=9.27-9.17 (m, 1H), 8.56-8.47 (m, 1H), 7.71-7.64 (m, 1H), 7.33-7.29 (m, 1H), 7.28-7.22 (m, 1H), 7.12-7.02 (m, 1H), 4.55-4.50 (m, 1H), 4.46-4.25 (m, 3H), 4.24-4.09 (m, 1H), 4.08-3.96 (m, 1H), 3.71-3.57 (m, 1H), 3.53-3.41 (m, 1H), 3.29-3.08 (m, 4H), 3.06-2.79 (m, 4H), 2.53-2.39 (m, 1H), 2.33-1.74 (m, 13H), 1.61-1.43 (m, 2H), 1.37-1.19 (m, 17H), 1.02-0.68 (m, 6H). LCMS (ESI, M+1): m/z=817.5.

### Example 895

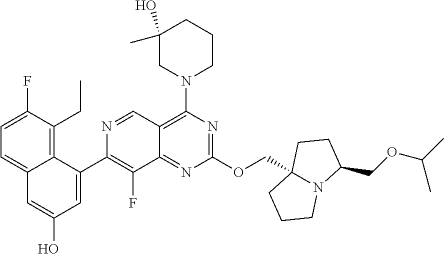 [SMILES: CCc1c(F)ccc2cc(O)cc(-c3ncc4c(N5CCC[C@@](C)(O)C5)nc(OC[C@@]56CCCN5[C@H](COC(C)C)CC6)nc4c3F)c12]

### (R)-1-(7-(8-ethyl-7-fluoro-3-hydroxynaphthalen-1-yl)-8-fluoro-2-(((3S,7aS)-3-(isopropoxymethyl)tetrahydro-1H-pyrrolizin-7a(5H)-yl)methoxy)pyrido[4,3-d]pyrimidin-4-yl)-3-methylpiperidin-3-ol

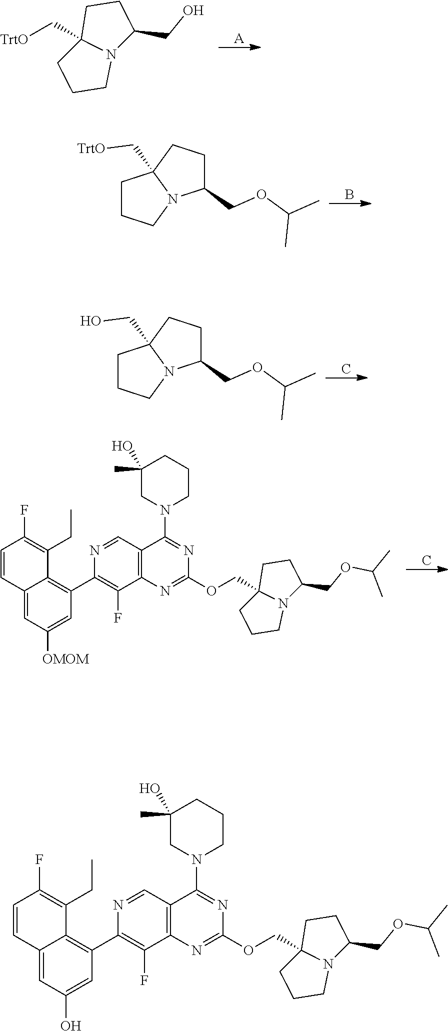

Step A. (3S,7aS)-3-(isopropoxymethyl)-7a-((trityloxy)methyl) hexahydro-1H-pyrrolizine: To a mixture of ((3S,7aS)-7a-((trityloxy)methyl) hexahydro-1H-pyrrolizin-3-yl)methanol (500 mg, 1.0 equiv) in THF (10 mL) was added NaH (483 mg, 60% purity, 10 equiv) at 0° C. The reaction was stirred at 0° C. for 0.5 hour. 2-iodopropane (4.11 g, 20 equiv) was added into above mixture. The reaction was stirred at 60° C. for 12 hours. The mixture was diluted with water (15 mL) and extracted with EtOAc (8 mL×3). The combined organic layers were dried over anhydrous sodium sulfate, concentrated, and purified with reversed phase flash chromatography [water (FA, 0.1%)/acetonitrile] to afford the title compound (90.0 mg, 16% yield) as yellow oil; LCMS (ESI, M+1): m/z=456.2.

Step B. ((3S,7aS)-3-(isopropoxymethyl) hexahydro-1H-pyrrolizin-7a-yl)methanol: A mixture of (3S,7aS)-3-(isopropoxymethyl)-7a-((trityloxy)methyl) hexahydro-1H-pyrrolizine (50.0 mg, 1.0 equiv) in TFA (1.54 g, 123. equiv) was stirred at 0° C. for 0.5 hour. The mixture was concentrated. The residue was diluted with MeOH. To the mixture was added NaHCO₃ (200 mg). The mixture was stirred at 25° C. for 0.5 hour. The mixture was filtered and concentrated to afford the title compound (125 mg, crude) as white solid.

Step C. (R)-1-(7-(8-ethyl-7-fluoro-3-(methoxymethoxy)naphthalen-1-yl)-8-fluoro-2-(((3S,7aS)-3-(isopropoxymethyl) hexahydro-1H-pyrrolizin-7a-yl)methoxy)pyrido[4,3-d]pyrimidin-4-yl)-3-methylpiperidin-3-ol: To a mixture of ((3S,7aS)-3-(isopropoxymethyl) hexahydro-1H-pyrrolizin-7a-yl)methanol (120 mg, 1.5 equiv) and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (198 mg, 1.0 equiv) in THF (1.0 mL) was added t-BuONa (1 M, 750 μL, 2.0 equiv) at −40° C. The reaction was stirred at 25° C. for 12 hours. The mixture was diluted with water (2 mL) and extracted with EtOAc (2 mL×3). The combined organic layers were dried over anhydrous sodium sulfate, concentrated, and purified with reversed phase flash chromatography [water (FA, 0.1%)/acetonitrile] to afford the title compound (32.0 mg, 12% yield) as yellow solid; LCMS (ESI, M+1): m/z=706.6.

Step D. (R)-1-(7-(8-ethyl-7-fluoro-3-hydroxynaphthalen-1-yl)-8-fluoro-2-(((3S. 7aS)-3-(isopropoxymethyl) hexahydro-1H-pyrrolizin-7a-yl)methoxy)pyrido[4,3-d]pyrimidin-4-yl)-3-methylpiperidin-3-ol: A mixture of (R)-1-(7-(8-ethyl-7-fluoro-3-(methoxymethoxy)naphthalen-1-yl)-8-fluoro-2-(((3S,7aS)-3-(isopropoxymethyl) hexahydro-1H-pyrrolizin-7a-yl)methoxy)pyrido[4,3-d]pyrimidin-4-yl)-3-methylpiperidin-3-ol (30.0 mg, 1.0 equiv) in HCl·MeOH (2 M, 1.5 mL, 70 equiv) was stirred at 0° C. for 0.5 hour. The mixture was concentrated. The residue was diluted with water (1 mL). The mixture was adjusted to pH=7 with saturated NaHCO₃ (10 mL) and extracted with ethyl acetate (2 mL×3). The combined organic layers were dried over anhydrous sodium sulfate, concentrated and purified with prep-HPLC (column: Phenomenex luna C18 150*25 mm*10 um; mobile phase: [water (FA)-ACN]; B %: 23%-53%, 10 min) to afford the title compound (3.03 mg, 10% yield, 0.27 HCOOH) as white solid. ¹H NMR (400 MHZ, METHANOL-d4) δ=9.22 (s, 1H), 7.68 (dd, J=5.2, 8.8 Hz, 1H), 7.37-7.19 (m, 2H), 7.06 (t, J=2.4 Hz, 1H), 4.61-4.44 (m, 2H), 4.42-4.25 (m, 2H), 3.67 (br d, J=4.8 Hz, 2H), 3.65-3.57 (m, 2H), 3.52-3.34 (m, 2H), 3.19-2.96 (m, 2H), 2.53-2.37 (m, 1H), 2.31-2.07 (m, 4H), 2.04-1.68 (m, 9H), 1.29 (d, J=9.6 Hz, 3H), 1.17 (dt, J=2.4, 4.0 Hz, 6H), 0.81 (td, J=7.2, 11.2 Hz, 3H); LCMS (ESI, M+1): m/z=662.3.

### Example 896

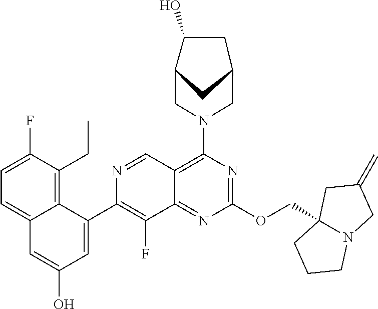 [SMILES: C=C1CN2CCC[C@@]2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-8-fluoro-2-(((S)-2-methylenetetrahydro-1H-pyrrolizin-7a(5H)-yl)methoxy)pyrido[4,3-d]pyrimidin-4-yl)-3-azabicyclo[3.2.1]octan-6-ol

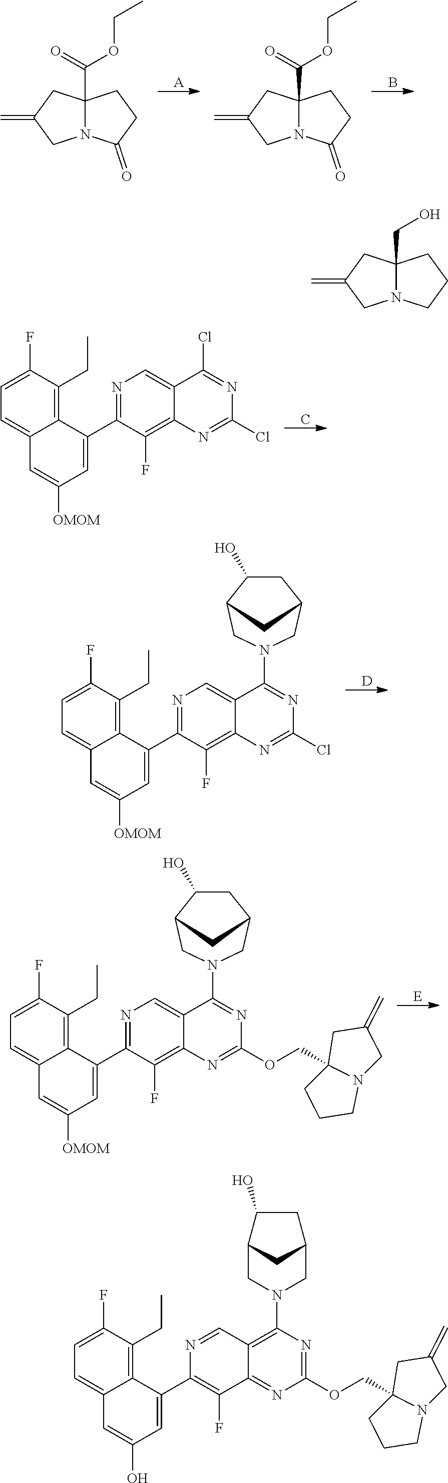

Step A. ethyl(S)-2-methylene-5-oxotetrahydro-1H-pyrrolizine-7a(5H)-carboxylate: Ethyl 2-methylene-5-oxotetrahydro-1H-pyrrolizine-7a(5H)-carboxylate (10 g) was separated by SFC [column: DAICEL CHIRALPAK IK (250 mm×50 mm, 10 um); mobile phase: CO₂-i-PrOH (0.1% NH₃H₂O); B %: 0%, isocratic elution mode] to afford the title compound (4.4 g, 41% yield) as light yellow gum; LCMS (ESI, M+1): m/z=210.1.

Step B. (S)-(2-methylenetetrahydro-1H-pyrrolizin-7a(5H)-yl)methanol: To a solution of ethyl(S)-2-methylene-5-oxotetrahydro-1H-pyrrolizine-7a(5H)-carboxylate (3.40 g, 1.0 equiv) in THF (34 mL) was added dropwise DIBAL-H (1M, 162 mL) at 0° C. The reaction was stirred at 0° C. for 5 hours. The mixture was quenched with water (8 mL), 15% NaOH (8 mL) and water (24 mL) at 0° C., and then diluted with EtOAc (500 mL). NaSO₄ (60 g) was added to the mixture. The mixture was stirred for 0.16 hours and filtered. The filter cake was washed with EtOAc (200 mL). The filtrate was concentrated and purified with column chromatography [Al₂O₃, petroleum ether/ethyl acetate=1/0 to 0/1] to afford the title compound (1.02 g, 41% yield) as light yellow oil; ¹H NMR (400 MHZ, CHLOROFORM-d) δ=4.92 (br d, J=10.4 Hz, 2H), 3.66 (br d, J=14.4 Hz, 1H), 3.36-3.22 (m, 3H), 3.17-3.06 (m, 1H), 2.73-2.61 (m, 1H), 2.51-2.45 (m, 1H), 2.36-2.30 (m, 1H), 1.96-1.85 (m, 2H), 1.85-1.66 (m, 2H).

Step C (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of 2,4-dichloro-7-(8-ethyl-7-fluoro-3-(methoxymethoxy)naphthalen-1-yl)-8-fluoropyrido[4,3-d]pyrimidine (1.26 g, 1.0 equiv) and DIEA (1.09 g, 3.0 equiv) in DCM (13 mL) was added a solution of (1R,5R,6R)-3-azabicyclo[3.2.1]octan-6-ol (356 mg, 1.0 equiv) in DCM (2 mL) dropwise at −40° C. The reaction was stirred at −40° C. for 1 hour. The mixture was quenched with water (40 mL) at 20° C. and extracted with DCM (2× 40 mL). The combined organic layers were washed with brine (40 mL), dried over anhydrous sodium sulfate, concentrated and purified with column chromatography [SiO₂, petroleum ether/ethyl acetate=10/1 to 1/1] to afford the title compound (1.38 g, 91% yield) as yellow solid; LCMS (ESI, M+1): m/z=541.2.

Step D. (1R,5R,6R)-3-(7-(8-ethyl-7-fluoro-3-(methoxymethoxy)naphthalen-1-yl)-8-fluoro-2-(((S)-2-methylenetetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (690 mg, 1.0 equiv) and(S)-(2-methylenetetrahydro-1H-pyrrolizin-7a(5H)-yl)methanol (586 mg, 3.0 equiv) in DMF (1 mL) was added DIEA (824 mg, 5.0 equiv). The reaction was stirred at 80° C. for 12 hours. The mixture was quenched with water (20 mL) at 20° C. and extracted with EtOAc (2×20 mL). The combined organic layers were washed with brine (20 mL), dried over anhydrous sodium sulfate, concentrated and purified with prep-HPLC [column: Phenomenex luna C18 150×40 mm×15 um; mobile phase: water (FA)-ACN; gradient: 22%-52% B over 15 min] to afford the title compound (400 mg, 47% yield) as white solid; LCMS (ESI, M+1): m/z=658.5.

Step E. (1R,5R,6R)-3-(7-(8-ethyl-7-fluoro-3-hydroxynaphthalen-1-yl)-8-fluoro-2-(((S)-2-methylenetetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((S)-2-methylenetetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) in MeOH (5 mL) was added HCl·MeOH (4M, 5 mL) at 0° C. The reaction was stirred at 0° C. for 6 hours. The mixture was concentrated, dissolved in MeOH (3 mL), adjusted to pH-7 with solid NaHCO₃ and filtered. The filtrate was concentrated and purified with prep-HPLC [column: C18 150×30 mm; mobile phase: water (FA)-ACN; gradient: 12%-42% B over 7 min] to afford the title compound (71.5 mg, 76% yield, 0.75 HCOOH) as white solid; ¹H NMR (400 MHZ, DMSO-d₆) δ=10.12-9.73 (m, 1H), 9.42-9.20 (m, 1H), 7.77 (br dd, J=6.0, 8.0 Hz, 1H), 7.44-7.27 (m, 2H), 7.13-6.98 (m, 1H), 4.93 (br s, 2H), 4.89-4.77 (m, 1H), 4.76-4.66 (m, 1H), 4.63-4.54 (m, 1H), 4.24-4.09 (m, 2H), 4.05-3.96 (m, 1H), 3.80-3.70 (m, 1H), 3.63-3.56 (m, 1H), 3.28-3.15 (m, 2H), 3.08-2.97 (m, 1H), 2.65-2.57 (m, 2H), 2.42-2.30 (m, 3H), 2.21-2.04 (m, 3H), 2.03-1.95 (m, 1H), 1.93-1.75 (m, 3H), 1.75-1.60 (m, 2H), 1.32-1.21 (m, 1H), 0.80-0.68 (m, 3H); LCMS (ESI, M+1): m/z=614.4.

### Example 897

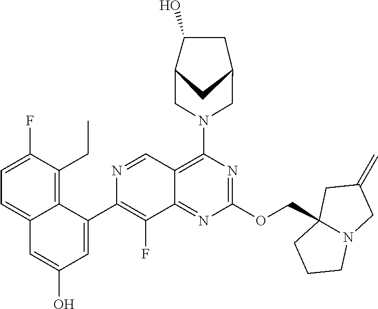 [SMILES: C=C1CN2CCC[C@]2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-8-fluoro-2-(((R)-2-methylenetetrahydro-1H-pyrrolizin-7a(5H)-yl)methoxy)pyrido[4,3-d]pyrimidin-4-yl)-3-azabicyclo[3.2.1]octan-6-ol

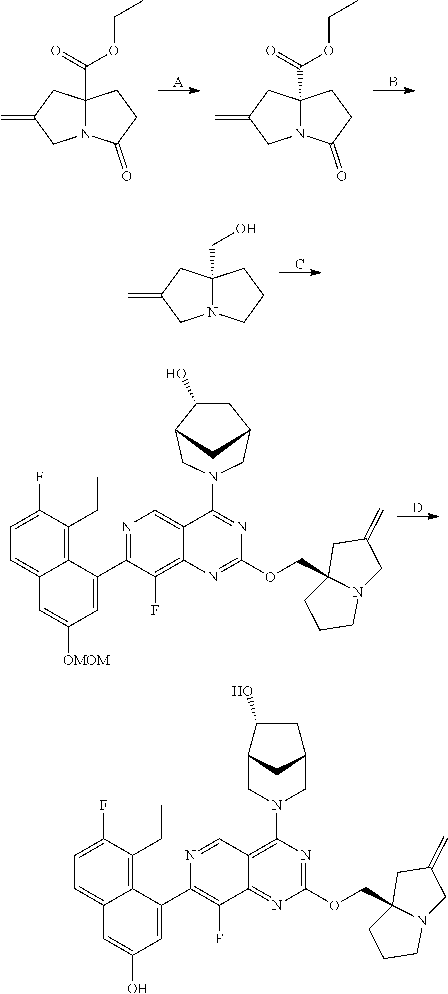

Step A. ethyl (R)-2-methylene-5-oxotetrahydro-1H-pyrrolizine-7a(5H)-carboxylate: Ethyl 2-methylene-5-oxotetrahydro-1H-pyrrolizine-7a(5H)-carboxylate (10 g) was separated by SFC [column: DAICEL CHIRALPAK IK (250 mm×50 mm, 10 μm); mobile phase: CO₂-i-PrOH (0.1% NH₃·H₂O); B %: 0%, isocratic elution mode] to afford the title compound (4.40 g, 42% yield) as light yellow gum; LCMS (ESI, M+1): m/z=210.1.

Step B. (R)-(2-methylenetetrahydro-1H-pyrrolizin-7a(5H)-yl)methanol: To a solution of ethyl (R)-2-methylene-5-oxotetrahydro-1H-pyrrolizine-7a(5H)-carboxylate (3.40 g, 1.0 equiv) in THF (34 mL) was added dropwise DIBAL-H (1M, 162 mL) at 0° C. The reaction was stirred at 0° C. for 5 hours. The mixture was quenched with water (8 mL), 15% NaOH (8 mL) and water (24 mL) at 0° C., and then diluted with EtOAc (500 mL). anhydrous NaSO₄ (60 g) was added to the mixture. The mixture stirred for 0.16 hours and filtered. The filter cake was washed with EtOAc (200 mL). The filtrate was concentrated and purified with column chromatography [Al₂O₃, petroleum ether/ethyl acetate=1/0 to 0/1] to afford the title compound (790 mg, 32% yield) as light yellow oil; ¹H NMR (400 MHZ, CHLOROFORM-d) δ=4.91 (br d, J=10.8 Hz, 2H), 3.64 (br d, J=14.4 Hz, 1H), 3.33-3.23 (m, 3H), 3.14-3.04 (m, 1H), 2.69-2.62 (m, 1H), 2.50-2.43 (m, 1H), 2.37-2.29 (m, 1H), 1.96-1.84 (m, 2H), 1.83-1.64 (m, 2H).

Step C. (1R,5R,6R)-3-(7-(8-ethyl-7-fluoro-3-(methoxymethoxy)naphthalen-1-yl)-8-fluoro-2-(((R)-2-methylenetetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (690 mg, 1.0 equiv) and (R)-(2-methylenetetrahydro-1H-pyrrolizin-7a(5H)-yl)methanol (586 mg, 3.0 equiv) in DMF (1 mL) was added DIEA (824 mg, 5.0 equiv). The reaction was stirred at 80° C. for 12 hours. The mixture was quenched with water (20 mL) at 20° C. and extracted with EtOAc (2×20 mL). The combined organic layers were washed with brine (20 mL), dried over anhydrous sodium sulfate, concentrated and purified with prep-HPLC [column: Phenomenex luna C18 150×40 mm×15 um; mobile phase: water (FA)-ACN; gradient: 22%-52% B over 15 min] to afford the title compound (500 mg, 59% yield) as a white solid; LCMS (ESI, M+1): m/z=658.4.

Step D. (1R,5R,6R)-3-(7-(8-ethyl-7-fluoro-3-hydroxynaphthalen-1-yl)-8-fluoro-2-(((R)-2-methylenetetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((R)-2-methylenetetrahydro-1H-pyrrolizin-7a(5H)-yl)methoxy)pyrido[4,3-d]pyrimidin-4-yl)-3-azabicyclo[3.2.1]octan-6-ol (100 mg, 1.0 equiv) in MeOH (5 mL) was added HCl·MeOH (4M, 5 mL) at 0° C. The reaction was stirred at 0° C. for 6 hours. The mixture was concentrated, dissolved in MeOH (3 mL), adjusted to pH=7 with solid NaHCO₃ and filtered. The filtrate was concentrated and purified with prep-HPLC [column: C18 150×30 mm; mobile phase: water (FA)-ACN; gradient: 12%-42% B over 7 min] to afford the title compound (37.0 mg, 39% yield, 0.60 HCOOH) as white solid; ¹H NMR (400 MHZ, DMSO-d₆) δ=10.23-9.72 (m, 1H), 9.36-9.23 (m, 1H), 7.76 (dd, J=6.0, 9.2 Hz, 1H), 7.38-7.30 (m, 2H), 7.03 (dd, J=2.4, 14.4 Hz, 1H), 4.90 (br s, 2H), 4.86-4.76 (m, 1H), 4.75-4.66 (m, 1H), 4.62-4.54 (m, 1H), 4.20-4.11 (m, 1H), 4.08-3.97 (m, 2H), 3.78-3.68 (m 1H), 3.57 (br d, J=14.0 Hz, 1H), 3.21 (br d, J=14.4 Hz, 2H), 3.04-2.98 (m, 1H), 2.64-2.56 (m, 2H), 2.36 (br d, J=18.4 Hz, 3H), 2.19-2.02 (m, 3H), 2.01-1.94 (m, 1H), 1.91-1.73 (m, 3H), 1.72-1.61 (m, 2H), 1.24 (br d, J=11.6 Hz, 1H), 0.77-0.65 (m, 3H); LCMS (ESI, M+1): m/z=614.4.

### Example 898

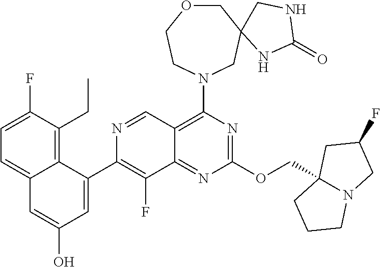 [SMILES: CCc1c(F)ccc2cc(O)cc(-c3ncc4c(N5CCOCC6(CNC(=O)N6)C5)nc(OC[C@@]56CCCN5C[C@H](F)C6)nc4c3F)c12]

### 10-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-7-oxa-1,3,10-triazaspiro[4.6]undecan-2-one

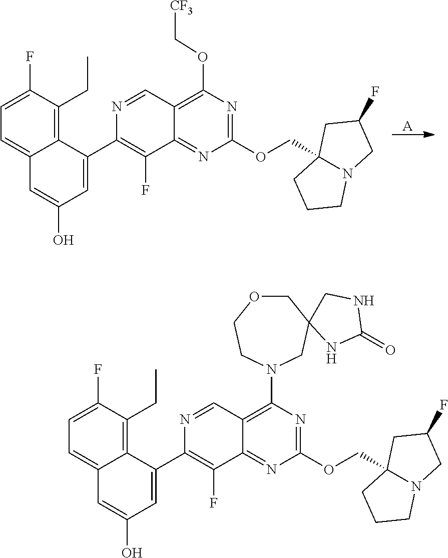

Step A. 10-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-7-oxa-1,3,10-triazaspiro[4.6]undecan-2-one: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (80 mg, 1.0 equiv) and 7-oxa-1,3,10-triazaspiro[4.6]undecan-2-one (34.7 mg, 1.5 equiv) in DMF (1 mL) were added K₃PO₄ (86 mg, 3.0 equiv) and 4 Å molecular sieve (80 mg). The reaction was stirred at 60° C. for 16 hours. The mixture was filtered and purified by prep-HPLC [column: Phenomenex luna C18×150×25 mm×10 um; mobile phase: water (FA)-ACN; B %: 10%-40%, 10 min] to afford the title compound (25.9 mg, 28% yield, HCOOH) as yellow solid; ¹H NMR (400 MHZ, METHANOL-d₄) δ=9.34-9.22 (m, 1H), 7.72-7.64 (m, 1H), 7.32 (d, J=2.4 Hz, 1H), 7.29-7.21 (m, 1H), 7.05 (dd, J=2.4, 5.6 Hz, 1H), 5.60-5.38 (m, 1H), 5.24-5.11 (m, 1H), 4.65-4.58 (m, 1H), 4.52-4.43 (m, 1H), 4.42-4.37 (m, 1H), 4.29-4.18 (m, 2H), 4.16-4.09 (m, 1H), 4.01 (ddd, J=5.6, 8.4, 14.0 Hz, 1H), 3.86-3.73 (m, 3H), 3.72-3.54 (m, 3H), 3.43-3.34 (m, 2H), 2.59-2.40 (m, 3H), 2.34-2.27 (m, 1H), 2.25-2.15 (m, 3H), 2.13-2.05 (m, 1H), 0.87-0.76 (m, 3H); LCMS (ESI, M+1): m/z=664.4.

### Example 899

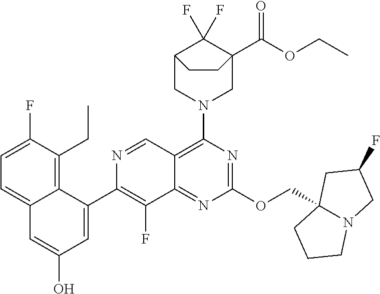 [SMILES: CCOC(=O)C12CCC(CN(c3nc(OC[C@@]45CCCN4C[C@H](F)C5)nc4c(F)c(-c5cc(O)cc6ccc(F)c(CC)c56)ncc34)C1)C2(F)F]

### ethyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-3-azabicyclo[3.2.1]octane-1-carboxylate

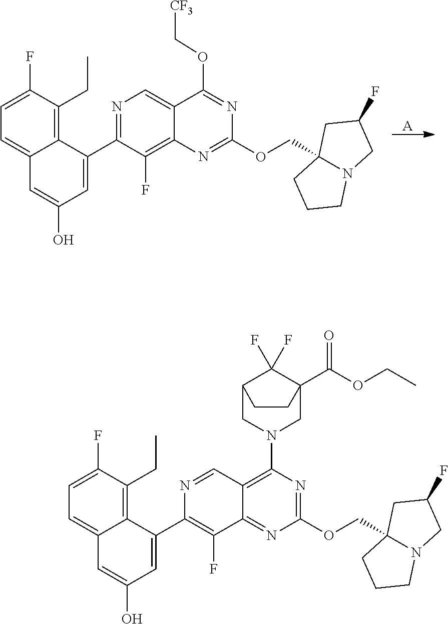

Step A. ethyl 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-3-azabicyclo[3.2.1]octane-1-carboxylate: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (130 mg, 1.0 equiv) and ethyl 8,8-difluoro-3-azabicyclo[3.2.1]octane-1-carboxylate (168 mg, 3.0 equiv, HCl) in DMF (1.3 mL) were added 4 Å molecular sieve (75.0 mg) and K₃PO₄ (140 mg, 3.0 equiv) in one portion under N₂. The reaction was stirred at 40° C. for 12 hours. The mixture was diluted with water (20 mL) and extracted with ethyl acetate (3×30 mL). The combined organic layers were washed with brine (30 mL), dried over Na₂SO₄, filtered, concentrated under reduced pressure and purified by prep-HPLC [column: Phenomenex Luna C18 150×25 mm×10 um; mobile phase: water (FA)-ACN; B %: 26%-56%, 10 min] to afford the title compound (14.2 mg, 8.6% yield, HCOOH) as an off-white solid; ¹H NMR (400 MHZ, METHANOL-d₄) δ=9.12 (s, 1H), 7.72-7.65 (m, 1H), 7.31 (d, J=2.6 Hz, 1H), 7.25 (t, J=9.4 Hz, 1H), 7.08-7.02 (m, 1H), 5.45-5.26 (m, 1H), 5.04-4.95 (m, 1H), 4.77-4.67 (m, 1H), 4.43-4.34 (m, 2H), 4.31-4.24 (m, 2H), 4.16-4.01 (m, 2H), 3.45-3.37 (m, 1H), 3.15-3.06 (m, 1H), 2.70-2.70 (m, 1H), 2.65-2.55 (m, 1H), 2.49-2.39 (m, 2H), 2.37-2.27 (m, 2H), 2.23-2.14 (m, 2H), 2.11-1.89 (m, 6H), 1.71-1.59 (m, 1H), 1.32 (t, J=7.2 Hz, 3H), 0.83-0.76 (m, 3H); LCMS (ESI, M+1): m/z=712.2.

### Example 900

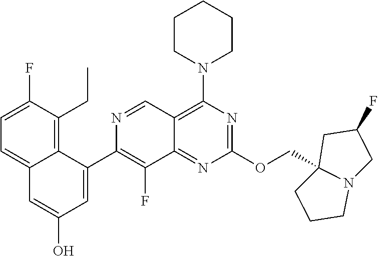 [SMILES: CCc1c(F)ccc2cc(O)cc(-c3ncc4c(N5CCCCC5)nc(OC[C@@]56CCCN5C[C@H](F)C6)nc4c3F)c12]

### 5-ethyl-6-fluoro-4-(8-fluoro-2-(((2R,7aS)-2-fluorotetrahydro-1H-pyrrolizin-7a(5H)-yl)methoxy)-4-(piperidin-1-yl)pyrido[4,3-d]pyrimidin-7-yl)naphthalen-2-ol

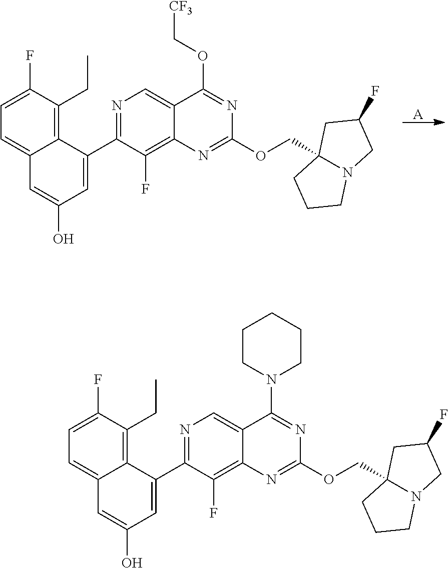

Step A. 5-ethyl-6-fluoro-4-(8-fluoro-2-(((2R,7aS)-2-fluorotetrahydro-1H-pyrrolizin-7a(5H)-yl)methoxy)-4-(piperidin-1-yl)pyrido[4,3-d]pyrimidin-7-yl)naphthalen-2-ol: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (260 mg, 1.0 equiv) and 4 Å molecular sieve (100 mg) in DMF (1 mL) was added piperidine (299 mg, 8.0 equiv). The reaction was stirred at 25° C. for 1 hour. The mixture was filtered and purified by reversed phase flash chromatography [C18, 0.1% formic acid condition] and prep-HPLC [column: Phenomenex luna C18 150×25 mm×10 μm; A: water (FA), B: ACN, B %: 23%-53% over 10 min] to afford the title compound (62.8 mg, 24% yield, 0.26 HCOOH) as off-white solid; ¹H NMR (400 MHZ, METHANOL-d₄) δ=9.03 (s, 1H), 7.68 (dd, J=6.0, 9.2 Hz, 1H), 7.33-7.18 (m, 2H), 7.06 (d, J=2.8 Hz, 1H), 5.47-5.27 (m, 1H), 4.47-4.28 (m, 2H), 4.06 (br s, 4H), 3.55-3.35 (m, 3H), 3.12 (dt, J=6.0, 9.6 Hz, 1H), 2.54-2.28 (m, 3H), 2.26-2.13 (m, 2H), 2.12-2.03 (m, 2H), 2.02-1.91 (m, 1H), 1.85 (br s, 6H), 0.80 (dt, J=1.2, 7.2 Hz, 3H); LCMS (ESI, M+1): m/z=578.4.

### Example 901

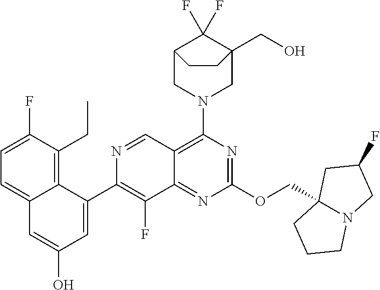 [SMILES: CCc1c(F)ccc2cc(O)cc(-c3ncc4c(N5CC6CCC(CO)(C5)C6(F)F)nc(OC[C@@]56CCCN5C[C@H](F)C6)nc4c3F)c12]

### 4-(4-(8,8-difluoro-1-(hydroxymethyl)-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

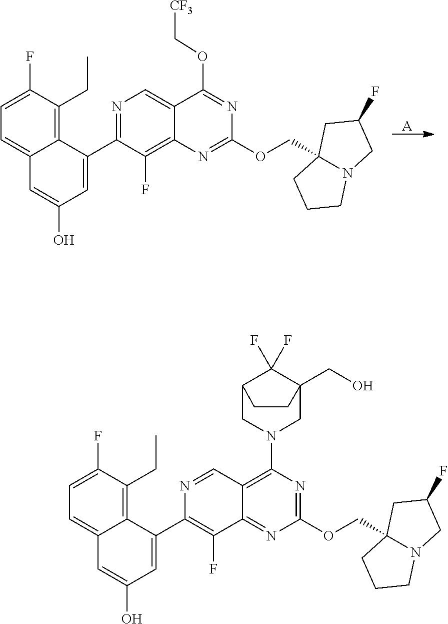

Step A. 4-(4-(8,8-difluoro-1-(hydroxymethyl)-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (200 mg, 1.0 equiv) and (8,8-difluoro-3-azabicyclo[3.2.1]octan-1-yl)methanol (179 mg, 3.0 equiv) in DMF (2 mL) were added 4 Å molecular sieve (50.0 mg) and K₃PO₄ (215 mg, 3.0 equiv) in one portion under N₂. The reaction was stirred at 40° C. for 12 hours. The mixture was filtered and purified by prep-HPLC [column: Phenomenex luna C18 150× 25 mm×10 um; mobile phase: water (FA)-ACN; B %: 25%-45%, 58 min] to afford the title compound (57.6 mg, 24.21% yield, HCOOH) as yellow solid; ¹H NMR (400 MHZ, METHANOL-d₄) δ=9.16 (d, J=4.0 Hz, 1H), 7.71-7.66 (m, 1H), 7.31 (d, J=2.8 Hz, 1H), 7.26 (t, J=9.6 Hz, 1H), 7.06 (d, J=2.4 Hz, 1H), 5.56-5.37 (d, J=52.8 Hz, 1H), 5.01-4.89 (m, 2H), 4.59-4.47 (m, 2H), 4.03-3.93 (m, 1H), 3.92-3.81 (m, 1H), 3.79 (s, 2H), 3.75 (m, 1H), 3.68-3.63 (m, 1H), 3.61-3.55 (m, 1H), 3.28 (br s, 1H), 2.63-2.51 (m, 2H), 2.48 (br s, 1H), 2.44-2.37 (m, 1H), 2.36-2.28 (m, 1H), 2.24-2.15 (m, 3H), 2.12-2.01 (m, 1H), 2.00-1.88 (m, 1H), 1.85-1.74 (m, 2H), 1.72-1.59 (m, 1H), 0.84-0.76 (m, 3H); F NMR (400 MHZ, METHANOL-d₄) δ=−72.48, −123.05, −173.72; LCMS (ESI, M+1): m/z=670.1.

### Example 902

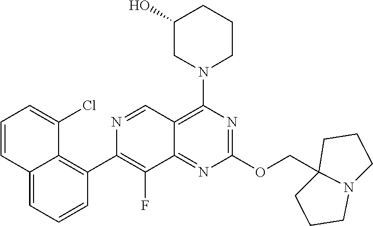 [SMILES: O[C@@H]1CCCN(c2nc(OCC34CCCN3CCC4)nc3c(F)c(-c4cccc5cccc(Cl)c45)ncc23)C1]

### (R)-1-(7-(8-chloronaphthalen-1-yl)-8-fluoro-2-((tetrahydro-1H-pyrrolizin-7a(5H)-yl)methoxy)pyrido[4,3-d]pyrimidin-4-yl) piperidin-3-ol

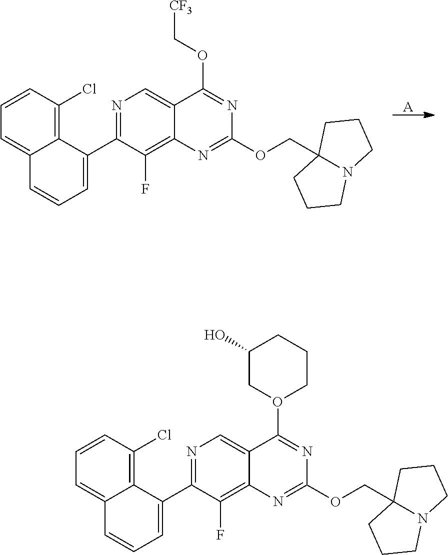

Step A. (R)-1-(7-(8-chloronaphthalen-1-yl)-8-fluoro-2-((tetrahydro-1H-pyrrolizin-7a(5H)-yl)methoxy)pyrido[4,3-d]pyrimidin-4-yl) piperidin-3-ol: To a mixture of 7-(8-chloronaphthalen-1-yl)-8-fluoro-2-((tetrahydro-1H-pyrrolizin-7a(5H)-yl)methoxy)-4-(2,2,2-trifluoroethoxy)pyrido[4,3-d]pyrimidine (70.0 mg, 1.0 equiv), (R)-piperidin-3-ol (38.8 mg, 3.0 equiv) and 4 Å molecular sieve (30 mg) in DMF (1 mL) was added DIEA (49.6 mg, 3.0 equiv). The reaction was stirred at 40° C. for 1 hour. The mixture was filtered and purified by reversed phase flash chromatography [C18, 0.1% formic acid condition] and prep-HPLC [column: Phenomenex luna C18 150×25 mm×10 μm; A: water (FA), B: ACN, B %: 12%-42% over 10 min] to afford the title compound (12.5 mg, 17% yield, 0.42HCOOH) as white solid; ¹H NMR (400 MHZ, METHANOL-d₄) δ=9.15 (d, J=6.0 Hz, 1H), 8.18-8.07 (m, 1H), 8.01 (d, J=8.4 Hz, 1H), 7.75-7.65 (m, 1H), 7.63-7.55 (m, 2H), 7.55-7.44 (m, 1H), 4.46 (s, 2H), 4.25 (br d, J=13.2 Hz, 1H), 4.10-3.94 (m, 3H), 3.92-3.79 (m, 1H), 3.43-3.34 (m, 2H), 3.04-2.92 (m, 2H), 2.24-2.14 (m, 2H), 2.13-1.97 (m, 6H), 1.96-1.87 (m, 2H), 1.81-1.71 (m, 2H); LCMS (ESI, M+1): m/z=548.3.

### Example 903

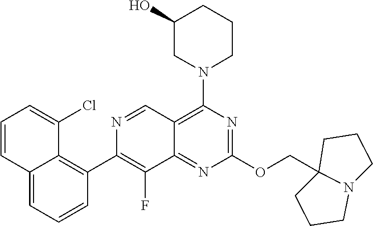 [SMILES: O[C@H]1CCCN(c2nc(OCC34CCCN3CCC4)nc3c(F)c(-c4cccc5cccc(Cl)c45)ncc23)C1]

### (S)-1-(7-(8-chloronaphthalen-1-yl)-8-fluoro-2-((tetrahydro-1H-pyrrolizin-7a(5H)-yl)methoxy)pyrido[4,3-d]pyrimidin-4-yl) piperidin-3-ol

Synthesized according to Example 902. The title compound was obtained as white solid (FA salt). ¹H NMR (400 MHZ, METHANOL-d₄) δ=9.15 (d, J=6.0 Hz, 1H), 8.20-8.08 (m, 1H), 8.04-7.96 (m, 1H), 7.73-7.65 (m, 1H), 7.63-7.57 (m, 2H), 7.54-7.48 (m, 1H), 4.43 (s, 2H), 4.31-4.18 (m, 1H), 4.10-3.94 (m, 3H), 3.91-3.77 (m, 1H), 3.38-3.32 (m, 2H), 3.01-2.88 (m, 2H), 2.25-2.13 (m, 2H), 2.12-1.94 (m, 6H), 1.94-1.83 (m, 2H), 1.81-1.70 (m, 2H); LCMS (ESI, M+1): m/z=548.3.

### Example 904

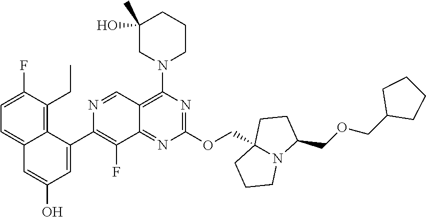 [SMILES: CCc1c(F)ccc2cc(O)cc(-c3ncc4c(N5CCC[C@@](C)(O)C5)nc(OC[C@@]56CCCN5[C@H](COCC5CCCC5)CC6)nc4c3F)c12]

### (R)-1-(2-(((3S,7aS)-3-((cyclopentylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

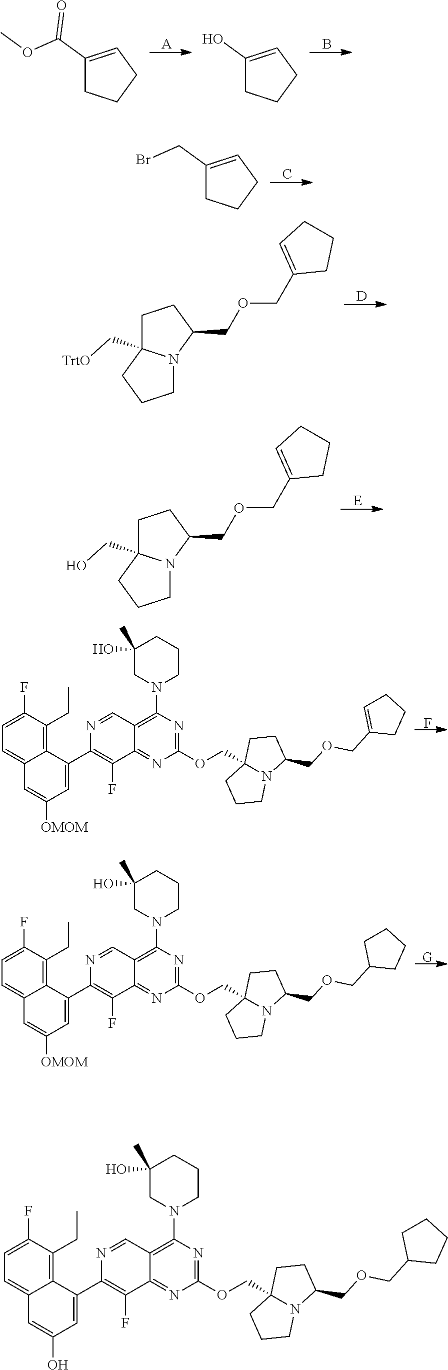

Step A. cyclopent-1-en-1-ylmethanol: To a solution of methyl cyclopent-1-ene-1-carboxylate (5.0 g, 1.0 equiv) in THF (50 mL) was added DIBAL-H (1 M, 43.6 mL, 1.1 equiv). The reaction was stirred at −78° C. for 3 hours. After completion, the mixture was diluted with THF (100 mL), warmed up to 0° C., quenched with H₂O (1.75 mL), 15% NaOH aqueous solution (1.75 mL) and H₂O (4.36 mL). The mixture was dried over anhydrous magnesium sulfate, filtered, concentrated under reduced pressure and purified by silica gel column chromatography (petroleum ether/ethyl acetate 10:1 to 5:1) to afford the title compound (1.1 g, 27% yield) as colorless oil; ¹H NMR (400 MHZ, CHLOROFORM-d) δ=5.61 (br d, J=1.6 Hz, 1H), 4.18 (s, 2H), 2.39-2.27 (m, 4H), 1.97-1.86 (m, 2H), 1.72 (br s, 1H).

Step B. 1-(bromomethyl)cyclopent-1-ene: To a solution of cyclopent-1-en-1-ylmethanol (500 mg, 1.0 equiv) in DCM (5 mL) was added PBr₃ (5.52 g, 4.0 equiv). The reaction was stirred at 0° C. for 2 hours. The mixture was quenched with ice water (5 mL), saturated NaHCO₃ (10 mL) and extracted with dichloromethane (2×10 mL). The combined organic layers were dried over Na₂SO₄, filtered and concentrated to afford the title compound (300 mg, crude) as green oil.

Step C. (3S,7aS)-3-((cyclopent-1-en-1-ylmethoxy)methyl)-7a-((trityloxy)methyl) hexahydro-1H-pyrrolizine: To a solution of ((3S,7aS)-7a-((trityloxy)methyl) hexahydro-1H-pyrrolizin-3-yl)methanol (514 mg, 1.0 equiv) in THF (3 mL) and DMF (3 mL) was added NaH (99.4 mg, 60% purity, 2.0 equiv). The reaction was stirred at 0° C. for 0.5 hours. 1-(bromomethyl)cyclopentene (300 mg, 1.5 equiv) was added and the reaction was stirred at 0-25° C. for 11.5 hours. The mixture was quenched with H₂O (5 mL) and extracted with ethyl acetate (2×15 mL). The combined organic layers were dried over Na₂SO₄, filtered, concentrated in vacuum and purified by reversed phase flash chromatography [C18, 0.1% formic acid condition] to afford the title compound (260 mg, 41% yield) as yellow oil; LCMS (ESI, M+1): m/z=494.4.

Step D. ((3S,7aS)-3-((cyclopent-1-en-1-ylmethoxy)methyl)tetrahydro-1H-pyrrolizin-7a(5H)-yl)methanol: To a solution of (3S,7aS)-3-((cyclopent-1-en-1-ylmethoxy)methyl)-7a-((trityloxy)methyl) hexahydro-1H-pyrrolizine (260 mg, 1.0 equiv) in DCM (3 mL) was added TFA (601 mg, 10 equiv). The reaction was stirred at 0-25° C. for 16 hours. The mixture was concentrated, dissolved in MeOH (3 mL), neutralized with solid NaHCO₃.and filtered. The filtrate was concentrated under vacuum and purified by column chromatography (Al₂O₃, Petroleum ether:Ethyl acetate=10:1 to 0:1 to Dichloromethane:Methanol=20:1) to afford the title compound (60 mg, 41% yield) as yellow oil; ¹H NMR (400 MHZ, CHLOROFORM-d) δ=5.64 (br s, 1H), 4.06 (s, 2H), 3.72-3.62 (m, 2H), 3.09-3.01 (m, 1H), 2.88-2.73 (m, 2H), 2.10-1.95 (m, 6H), 1.91 (br d, J=7.6 Hz, 2H), 1.83 (br dd, J=4.0, 8.8 Hz, 6H), 1.61-1.57 (m, 2H).

Step E. (R)-1-(2-(((3S,7aS)-3-(cyclopent-1-en-1-ylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of ((3S,7aS)-3-((cyclopent-1-en-1-ylmethoxy)methyl)tetrahydro-1H-pyrrolizin-7a(5H)-yl)methanol (60 mg, 1.0 equiv) and (R)-1-(2-chloro-7-(8-ethyl-7-fluoro-3-(methoxymethoxy)naphthalen-1-yl)-8-fluoropyrido[4,3-d]pyrimidin-4-yl)-3-methylpiperidin-3-ol (126 mg, 1.0 equiv) in toluene (1 mL) was added t-BuONa (2 M, 477 μL, 4.0 equiv). The reaction was stirred at 0° C. for 2 hours. The mixture was diluted with water (3 mL) and extracted with ethyl acetate (2×5 mL). The combined organic layers were dried over anhydrous sodium sulfate, filtered, concentrated under vacuum and purified by reversed phase flash chromatography [C18, 0.1% formic acid condition] to afford the title compound (60 mg, 24% yield) as yellow solid; LCMS (ESI, M+1): m/z=744.5.

Step F. (R)-1-(2-(((3S,7aS)-3-((cyclopentylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a solution of (R)-1-(2-(((3S,7aS)-3-((cyclopent-1-en-1-ylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (60 mg, 1.0 equiv) in MeOH (1 mL) was added Pd/C (10 mg, 10% purity) under N₂ atmosphere. The suspension was degassed and purged with H₂ for 3 times. The reaction was stirred under H₂ (15 Psi) at 25° C. for 1 hour. The mixture was filtered and concentrated to afford the title compound (30 mg, 29% yield.) as white solid; LCMS (ESI, M+1): m/z=746.5.

Step G. (R)-1-(2-(((3S,7aS)-3-((cyclopentylmethoxy)methyl)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a solution of (R)-1-(2-(((3S,7aS)-3-((cyclopentylmethoxy)methyl)tetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (30 mg, 1.0 equiv) in acetonitrile (0.5 mL) was added HCl·dioxane (4 M, 1 mL, 99 equiv). The reaction was stirred at 25° C. for 1 hour. The mixture was concentrated, dissolved in MeOH (1 mL), neutralized with solid NaHCO₃ and filtered. The filtrate was concentrated under vacuum and purified by prep-HPLC [column: Waters xbridge 150×25 mm×10 um; mobile phase: water (NH₄HCO₃)-ACN; B %: 52%-82%, 10 min] to afford the title compound (3.3 mg, 11% yield) as yellow solid; ¹H NMR (400 MHz, METHANOL-d₄) δ=9.24-9.13 (m, 1H), 7.72-7.60 (m, 1H), 7.35-7.19 (m, 2H), 7.09-6.99 (m, 1H), 5.03-4.97 (m, 1H), 4.71-4.62 (m, 2H), 4.43-4.22 (m, 3H), 3.72-3.53 (m, 3H), 3.52-3.40 (m, 2H), 3.38-3.33 (m, 1H), 3.26 (br s, 1H), 3.02-2.79 (m, 2H), 2.53-2.40 (m, 1H), 2.23-2.14 (m, 3H), 2.01 (br dd, J=2.8, 7.6 Hz, 1H), 1.86 (br dd, J=6.0, 11.2 Hz, 4H), 1.80-1.66 (m, 5H), 1.63-1.42 (m, 4H), 1.28 (br dd, J=3.6, 9.6 Hz, 6H), 0.88-0.71 (m, 3H); LCMS (ESI, M+1): m/z=702.2.

### Example 905

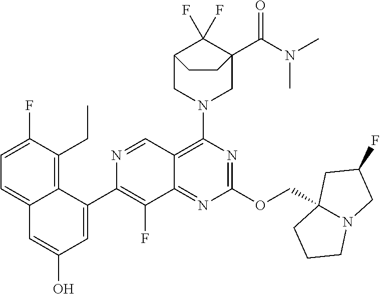 [SMILES: CCc1c(F)ccc2cc(O)cc(-c3ncc4c(N5CC6CCC(C(=O)N(C)C)(C5)C6(F)F)nc(OC[C@@]56CCCN5C[C@H](F)C6)nc4c3F)c12]

### 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-N,N-dimethyl-3-azabicyclo[3.2.1]octane-1-carboxamide

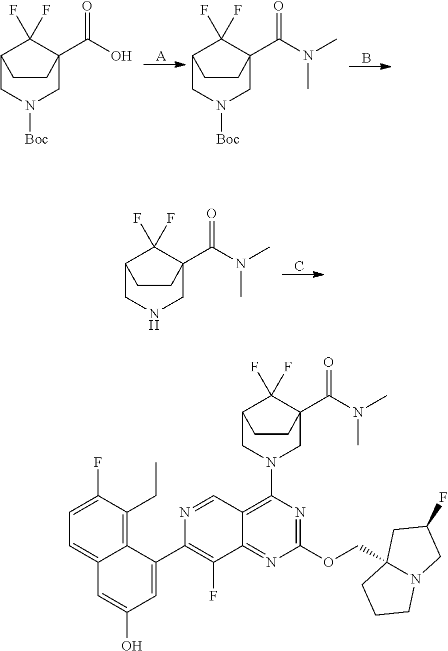

Step A. tert-butyl 1-(dimethylcarbamoyl)-8,8-difluoro-3-azabicyclo[3.2.1]octane-3-carboxylate: To a mixture of 3-(tert-butoxycarbonyl)-8,8-difluoro-3-azabicyclo[3.2.1]octane-1-carboxylic acid (200 mg, 1.0 equiv) and DIEA (266 mg, 3.0 equiv) in DMF (1 mL) was added HATU (392 mg, 1.5 equiv). After stirring at 25° C. for 0.5 hours, Me₂NH (2 M, 1.03 mL, 3.0 equiv) was added. The reaction was stirred for 2 hours under N₂ atmosphere. The mixture was concentrated under reduced pressure and purified by reversed phase flash chromatography [C18, 0.1% formic acid condition] to afford the title compound (140 mg, 64% yield) as a yellow solid; LCMS (ESI, M+1): m/z=318.9

Step B. 8,8-difluoro-N,N-dimethyl-3-azabicyclo[3.2.1]octane-1-carboxamide: To a solution of tert-butyl 1-(dimethylcarbamoyl)-8,8-difluoro-3-azabicyclo[3.2.1]octane-3-carboxylate (130 mg, 1.0 equiv) in MeOH (2.0 mL) was added HCl·MeOH (4 M, 2 mL, 19.6 equiv) at 0° C. The reaction was stirred at 0° C. for 1 hour. The mixture was concentrated under reduced pressure, diluted with MeOH (6 mL), adjusted to pH=10 by solid NaHCO₃, filtered and concentrated to afford the title compound (100 mg, crude) as yellow solid; LCMS (ESI, M+1): m/z=219.2.

Step C. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-N,N-dimethyl-3-azabicyclo[3.2.1]octane-1-carboxamide: To a mixture of 7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl 4-methylbenzenesulfonate (35.0 mg, 1.0 equiv) and 8,8-difluoro-N,N-dimethyl-3-azabicyclo[3.2.1]octane-1-carboxamide (38.7 mg, 3.0 equiv) in DMF (0.5 mL) were added K₃PO₄ (125 mg, 10 equiv) and 4 Å molecular sieve (10 mg, 1.0 equiv). The reaction was stirred at 60° C. for 16 hours. The mixture was concentrated under reduced pressure and purified by prep-HPLC [neutral condition; column: Phenomenex Luna C18 150×25 mm×10 um; mobile phase: water (NH₄HCO₃)-ACN; B %: 40%-70%, 10 min] to afford the title compound (15.1 mg, 35.4% yield) as white solid; ¹H NMR (400 MHZ, METHANOL-d4) δ=9.13-9.05 (m, 1H), 7.68 (dd, J=6.0, 9.2 Hz, 1H), 7.30 (d, J=2.8 Hz, 1H), 7.25 (t, J=9.6 Hz, 1H), 7.05 (dd, J=2.4, 12.8 Hz, 1H), 5.42-5.22 (m, 2H), 4.51-4.38 (m, 1H), 4.35-4.27 (m, 2H), 4.27-4.20 (m, 1H), 3.74 (br d, J=14.1 Hz, 1H), 3.29-3.22 (m, 3H), 3.18 (br d, J=6.0 Hz, 3H), 3.02 (dt, J=5.6, 9.2 Hz, 3H), 2.65-2.53 (m, 1H), 2.53-2.41 (m, 2H), 2.38-2.19 (m, 2H), 2.18-2.02 (m, 4H), 2.01-1.84 (m, 4H), 1.53-1.40 (m, 1H), 0.79 (td, J=7.6, 20.0 Hz, 3H); F NMR (400 MHZ, METHANOL-d4) δ=−114.08, −121.10, −124.04, −138.65, −173.55; LCMS (ESI, M+1): m/z=711.1.

### Example 906

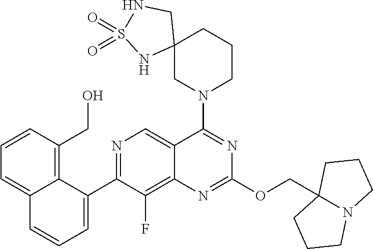 [SMILES: O=S1(=O)NCC2(CCCN(c3nc(OCC45CCCN4CCC5)nc4c(F)c(-c5cccc6cccc(CO)c56)ncc34)C2)N1]

### 7-(8-fluoro-7-(8-(hydroxymethyl)naphthalen-1-yl)-2-((tetrahydro-1H-pyrrolizin-7a(5H)-yl)methoxy)pyrido[4,3-d]pyrimidin-4-yl)-2-thia-1,3,7-triazaspiro[4.5]decane 2,2-dioxide

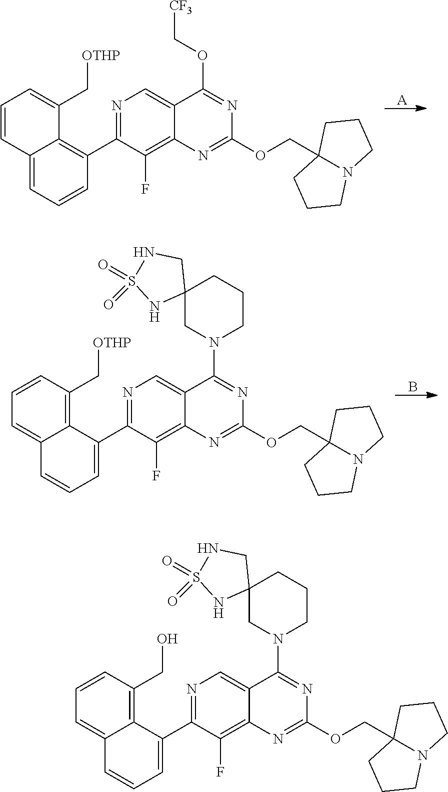

Step A. 7-(8-fluoro-2-((tetrahydro-1H-pyrrolizin-7a(5H)-yl)methoxy)-7-(8-(((tetrahydro-2H-pyran-2-yl)oxy)methyl)naphthalen-1-yl)pyrido[4,3-d]pyrimidin-4-yl)-2-thia-1,3,7-triazaspiro[4.5]decane 2,2-dioxide: To a mixture of 8-fluoro-2-((tetrahydro-1H-pyrrolizin-7a(5H)-yl)methoxy)-7-(8-(((tetrahydro-2H-pyran-2-yl)oxy)methyl)naphthalen-1-yl)-4-(2,2,2-trifluoroethoxy)pyrido[4,3-d]pyrimidine (180 mg, 1.0 equiv) in DMF (0.1 mL) were added rac-(R)-2-thia-1,3,7-triazaspiro[4.5]decane 2,2-dioxide (65.9 mg, 1.2 equiv), 4 Å molecular sieve (50 mg) and DIEA (371 mg, 500 μL, 10 equiv). The reaction was stirred at 40° C. for 12 hours. The mixture was filtered and purified by reversed phase flash chromatography [C18, 0.1% formic acid condition] to afford the title compound (80 mg, 37% yield) as a yellow solid; ¹H NMR (400 MHZ, CHLOROFORM-d) δ=9.10-9.00 (m, 1H), 8.00 (d, J=8.0 Hz, 1H), 7.96-7.88 (m, 1H), 7.75-7.39 (m, 4H), 4.55-4.22 (m, 5H), 4.11-4.03 (m, 1H), 4.02-3.85 (m, 1H), 3.87-3.59 (m, 2H), 3.58-3.39 (m, 2H), 3.34-3.12 (m, 4H), 2.73-2.64 (m, 2H), 2.20-2.07 (m, 3H), 2.02-1.85 (m, 5H), 1.82-1.59 (m, 5H), 1.55-1.31 (m, 5H); LCMS (ESI, M+1): m/z=718.3.

Step B. 7-(8-fluoro-7-(8-(hydroxymethyl)naphthalen-1-yl)-2-((tetrahydro-1H-pyrrolizin-7a(5H)-yl)methoxy)pyrido[4,3-d]pyrimidin-4-yl)-2-thia-1,3,7-triazaspiro[4.5]decane 2,2-dioxide: A solution of 7-(8-fluoro-2-((tetrahydro-1H-pyrrolizin-7a(5H)-yl)methoxy)-7-(8-(((tetrahydro-2H-pyran-2-yl)oxy)methyl)naphthalen-1-yl)pyrido[4,3-d]pyrimidin-4-yl)-2-thia-1,3,7-triazaspiro[4.5]decane 2,2-dioxide (60 mg, 1.0 equiv) and TFA (0.3 mL) was stirred at 0° C. for 1 hour. The mixture was added and diluted with acetonitrile (1.0 ml) and neutralized with solid NaHCO₃, filtered, and purified by prep-HPLC [column: UniSil 3-100 C18 Ultra 150×25 mm×3 μm; A: water (FA), B: ACN, B %: 17%-37% over 10 min], followed by prep-HPLC [column: Waters Xbridge BEH C18 150×25 mm×5 μm; A: water (NH₄HCO₃), B: ACN, B %: 25%-55% over 9 min] to afford the title compound (10 mg, 18.1% yield) as white solid; ¹H NMR (400 MHZ, METHANOL-d₄) δ=9.09 (s, 1H), 8.07 (d, J=8.4 Hz, 1H), 7.95 (d, J=8.4 Hz, 1H), 7.78-7.76 (m, 1H), 7.63-7.52 (m, 2H), 7.51-7.44 (m, 1H), 4.63-4.57 (m, 2H), 4.47-4.40 (m, 1H), 4.39-4.32 (m, 2H), 4.31-4.26 (m, 1H), 3.88-3.77 (m, 1H), 3.74-3.65 (m, 1H), 3.45-3.39 (m, 1H), 3.20 (dd, J=5.2, 12.0 Hz, 1H), 3.17-3.08 (m, 2H), 2.81-2.69 (m, 2H), 2.02-2.15 (m, 4H), 2.00-1.84 (m, 6H), 1.82-1.71 (m, 2H); LCMS (ESI, M+1): m/z=634.3.

### Example 907

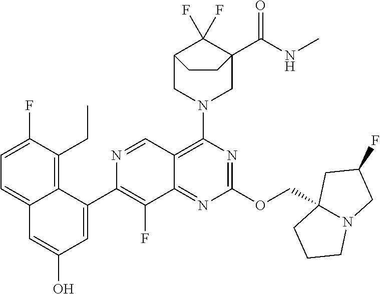 [SMILES: CCc1c(F)ccc2cc(O)cc(-c3ncc4c(N5CC6CCC(C(=O)NC)(C5)C6(F)F)nc(OC[C@@]56CCCN5C[C@H](F)C6)nc4c3F)c12]

### (1S,5R)-3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-N-methyl-3-azabicyclo[3.2.1]octane-1-carboxamide

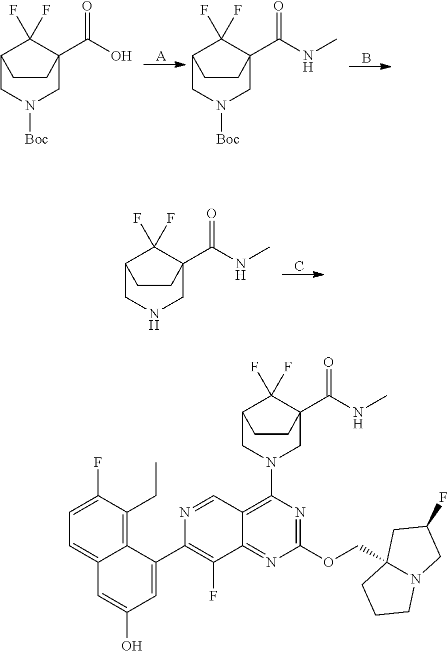

Step A. tert-butyl 8,8-difluoro-1-(methylcarbamoyl)-3-azabicyclo[3.2.1]octane-3-carboxylate: To a solution of 3-(tert-butoxycarbonyl)-8,8-difluoro-3-azabicyclo[3.2.1]octane-1-carboxylic acid (200 mg, 1.0 equiv) and DIEA (266 mg, 3.0 equiv) in DMF (2.0 mL) was added HATU (392 mg, 1.5 equiv) at 0-5° C. The reaction was stirred at 0-25° C. for 0.5 hours. To the mixture was added methanamine (2.0 M, 1.0 mL, 3.0 equiv) at 0° C. The reaction was stirred at 0-25° C. for 4 hours. The mixture was diluted with water (6.0 mL) and extracted with ethyl acetate (3× 3.0 mL). The combined organic layers were washed with brine, dried over anhydrous sodium sulfate, filtered, concentrated and purified by reversed phase flash chromatography [C18, 0.1% formic acid condition] to afford the title compound (100 mg, 41% yield) as light yellow solid; LCMS (ESI, M−55): m/z=249.0.

Step B. 8,8-difluoro-N-methyl-3-azabicyclo[3.2.1]octane-1-carboxamide: To a solution of tert-butyl 8,8-difluoro-1-(methylcarbamoyl)-3-azabicyclo[3.2.1]octane-3-carboxylate (100 mg, 1.0 equiv) in MeOH (1.0 mL) was added HCl·MeOH (4.0 M, 1.0 mL, 12 equiv) at 0-5° C. The reaction was stirred at 0-5° C. for 5 hours. The mixture was concentrated under reduced pressure, dissolved in MeOH (1.0 mL), adjusted to pH=9 with solid NaHCO₃ and filtered. The filtrate was concentrated under reduced pressure to afford the title compound (80 mg, crude) as yellow solid.

Step C. 3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-8,8-difluoro-N-methyl-3-azabicyclo[3.2.1]octane-1-carboxamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (40.0 mg, 1.0 equiv) and 8,8-difluoro-N-methyl-3-azabicyclo[3.2.1]octane-1-carboxamide (27.6 mg, 2.0 equiv) in DMF (1.0 mL) were added K₃PO₄ (71.7 mg, 5.0 equiv) and 4 Å molecular sieve (10.0 mg). The reaction was stirred at 40° C. for 12 hours. The mixture was filtered and purified by reversed-phase HPLC [column: Phenomenex luna C18 150×25 mm×10 um; mobile phase: water (FA)-ACN; B %: 16%-46%, 10 min] to afford the title compound (11.0 mg, 21% yield, HCOOH) as yellow solid; ¹H NMR (400 MHZ, METHANOL-d₄) δ=9.13 (s, 1H), 7.68 (dd, J=6.0, 9.2 Hz, 1H), 7.31 (d, J=2.4 Hz, 1H), 7.29-7.22 (m, 1H), 7.05 (t, J=3.2 Hz, 1H), 5.53-5.32 (m, 1H), 5.08-4.93 (m, 1H), 4.75 (dt, J=1.6, 10.8 Hz, 1H), 4.55-4.41 (m, 2H), 4.14-3.96 (m, 2H), 3.62-3.44 (m, 1H), 3.63-3.44 (m, 2H), 3.21 (dt, J=5.6, 10 Hz, 1H), 2.82 (d, J=3.6 Hz, 3H), 2.68-2.57 (m, 1H), 2.55-2.34 (m, 3H), 2.34-2.20 (m, 3H), 2.20-2.09 (m, 3H), 2.03 (br d, J=5.6 Hz, 3H), 0.85-0.74 (m, 3H); F NMR (400 MHZ, METHANOL-d4) δ=−112.440, −121.071, −128.148, −138.836, −173.900; LCMS (ESI, M+1): m/z=697.4.

### Example 908

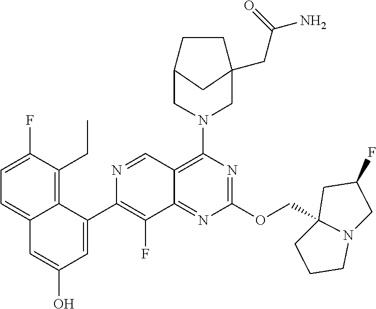 [SMILES: CCc1c(F)ccc2cc(O)cc(-c3ncc4c(N5CC6CCC(CC(N)=O)(C6)C5)nc(OC[C@@]56CCCN5C[C@H](F)C6)nc4c3F)c12]

### 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)acetamide

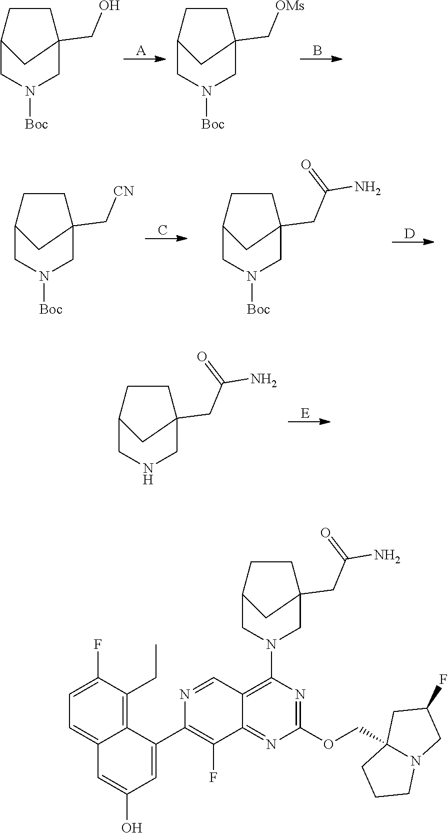

Step A. tert-butyl 1-(((methylsulfonyl)oxy)methyl)-3-azabicyclo[3.2.1]octane-3-carboxylate: To a solution of tert-butyl 1-(hydroxymethyl)-3-azabicyclo[3.2.1]octane-3-carboxylate (300 mg, 1.0 equiv) and TEA (3.77 g, 30 equiv) in THF (5.0 mL) was added methylsulfonyl methanesulfonate (2.17 g, 10 equiv). The reaction was stirred at 25° C. for 2 hours. The mixture was diluted with cold water (15 mL) and extracted with ethyl acetate (3×5.0 mL). The combined organic layers were washed with brine, dried over anhydrous sodium sulfate and concentrated to afford the title compound (397 mg, crude) as a yellow oil.

Step B. tert-butyl 1-(cyanomethyl)-3-azabicyclo[3.2.1]octane-3-carboxylate: To a solution of tert-butyl 1-(((methylsulfonyl)oxy)methyl)-3-azabicyclo[3.2.1]octane-3-carboxylate (397 mg, 1.0 equiv) in DMSO (6.0 mL) were added KI (309 mg, 1.5 equiv) and KCN (425 mg, 5.3 equiv). The reaction was stirred at 100° C. for 5 hours. The mixture was quenched with water (24 mL) at 25° C. and extracted with ethyl acetate (3×10 mL). The combined organic layers were dried over anhydrous sodium sulfate, filtered, concentrated and purified by column chromatography (SiO₂, Petroleum ether/Ethyl acetate=20/1 to 5/1) to afford the title compound (120 mg, 38% yield) as a yellow oil; ¹H NMR (400 MHZ, CDCl₃) δ=3.99-3.67 (m, 2H), 2.90-2.64 (m, 2H), 2.41 (s, 2H), 2.36-2.22 (m, 1H), 1.91-1.75 (m, 1H), 1.75-1.60 (m, 3H), 1.57-1.51 (m, 2H), 1.46 (s, 9H).

Step C. tert-butyl 1-(2-amino-2-oxoethyl)-3-azabicyclo[3.2.1]octane-3-carboxylate: To a solution of tert-butyl 1-(cyanomethyl)-3-azabicyclo[3.2.1]octane-3-carboxylate (50.0 mg, 1.0 equiv) and K₂CO₃ (55.2 mg, 2.0 equiv) in DMSO (1.3 mL) was added H₂O₂ (212 mg, 9.4 equiv). The reaction was stirred at 60° C. for 6 hours. The mixture was diluted with cold water (6.0 mL) and extracted with EtOAc (3×4.0 mL). The combined organic layers were washed with saturated Na₂SO₃ solution (2 mL), brine (2 mL), dried over Na₂SO₄, filtered and concentrated to afford the title compound (50.0 mg, crude) as a colorless oil.

Step D. 2-(3-azabicyclo[3.2.1]octan-1-yl)acetamide: To a solution of tert-butyl 1-(2-amino-2-oxoethyl)-3-azabicyclo[3.2.1]octane-3-carboxylate (50.0 mg, 1.0 equiv) in MeOH (0.50 mL) was added HCl·MeOH (4.0 M, 0.50 mL, 11 equiv). The reaction was stirred at 0-5° C. for 6 hours. The mixture was concentrated under reduced pressure, diluted with MeOH (2.0 mL), adjusted to pH=7-8 with solid NaHCO₃ and filtered. The filtrate was concentrated to afford the title compound (60 mg, crude) as a light yellow solid.

Step E. 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)acetamide: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)pyrido[4,3-d]pyrimidin-7-yl)naphthalen-2-ol (30.0 mg, 1.0 equiv) and 2-(3-azabicyclo[3.2.1]octan-1-yl)acetamide (17.7 mg, 2.0 equiv) in DMF (0.30 mL) were added 4 Å molecular sieve (20.0 mg) and K₃PO₄ (33.5 mg, 3.0 equiv). The reaction was stirred at 40° C. for 12 hours. The mixture was filtered and the filtrate was purified by prep-HPLC [column: Waters xbridge 150×25 mm 10 um; mobile phase: water (NH₄HCO₃)-ACN; B %: 37%-67%, 10 min] to afford the title compound (5.92 mg, 7.3% yield) as an off-white solid; ¹H NMR (400 MHZ, METHANOL-d₄) δ=9.08 (s, 1H), 7.75-7.62 (m, 1H), 7.30 (d, J=2.8 Hz, 1H), 7.25 (t, J=9.6 Hz, 1H), 7.12-7.03 (m, 1H), 5.41-5.24 (m, 1H), 5.07-4.98 (m, 2H), 4.39-4.22 (m, 2H), 3.72-3.61 (m, 1H), 3.52-3.40 (m, 2H), 3.26-3.16 (m, 2H), 3.08-2.97 (m, 1H), 2.53-2.45 (m, 2H), 2.42-2.34 (m, 2H), 2.25 (br d, J=16.4 Hz, 1H), 2.21-2.13 (m, 2H), 2.07-1.93 (m, 3H), 1.88 (br d, J=12.0 Hz, 2H), 1.79-1.71 (m, 2H), 1.69-1.60 (m, 1H), 1.59-1.48 (m, 1H), 1.35-1.28 (m, 1H), 0.86-0.74 (m, 3H); LCMS (ESI, M+1): m/z=661.2.

### Example 909

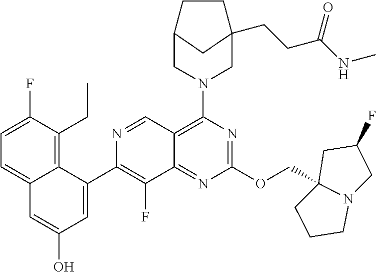 [SMILES: CCc1c(F)ccc2cc(O)cc(-c3ncc4c(N5CC6CCC(CCC(=O)NC)(C6)C5)nc(OC[C@@]56CCCN5C[C@H](F)C6)nc4c3F)c12]

### 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylpropanamide

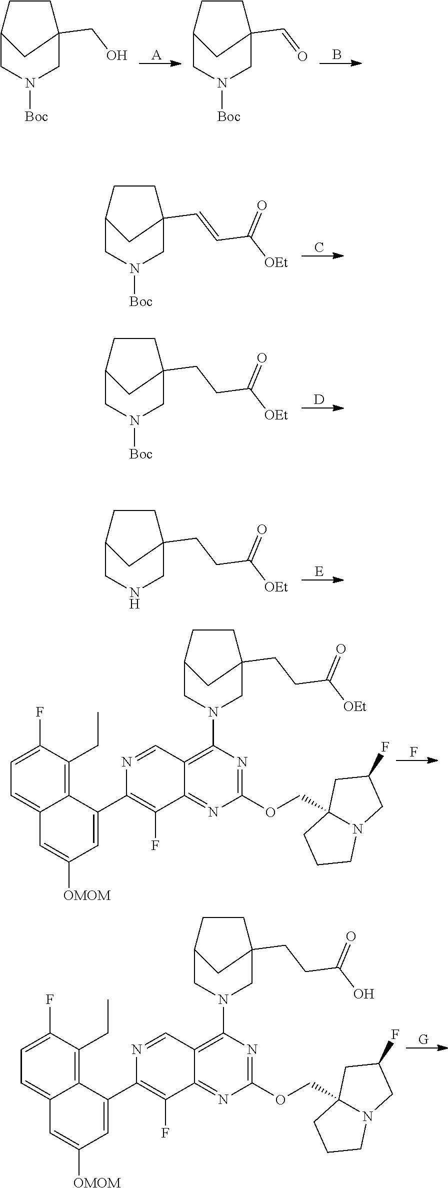

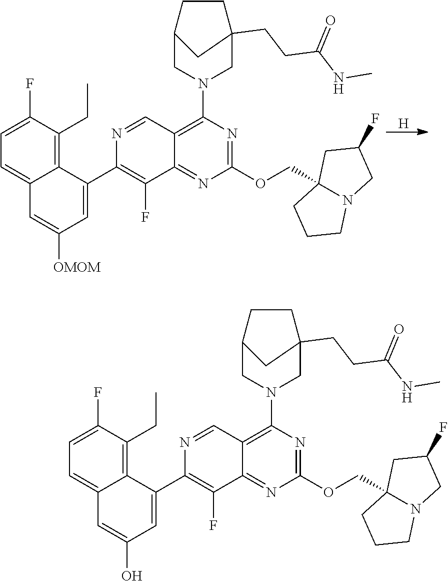

Step A. tert-butyl 1-formyl-3-azabicyclo[3.2.1]octane-3-carboxylate: To a solution of tert-butyl 1-(hydroxymethyl)-3-azabicyclo[3.2.1]octane-3-carboxylate (2.0 g, 1.0 equiv) in DCM (40 mL) was added DMP (5.27 g, 1.5 equiv). The reaction was stirred at 0° C. for 2 hours. The mixture was quenched with saturated aqueous NaHCO₃ (2 mL) and extracted with DCM (3×2 mL). The combined organic layers were dried over Na₂SO₄, concentrated and purified by silica gel chromatography (Petroleum ether/Ethyl acetate 50/1, 3/1) to afford the title compound (2 g, crude) as colorless oil.

Step B. tert-butyl(E)-1-(3-ethoxy-3-oxoprop-1-en-1-yl)-3-azabicyclo[3.2.1]octane-3-carboxylate: To a solution of ethyl 2-diethoxyphosphorylacetate (2.25 g, 1.2 equiv) in THF (20 mL) was added NaH (501 mg, 60% purity, 1.5 equiv) at 0° C. The reaction was stirred at 0° C. for 0.5 hours. tert-butyl 1-formyl-3-azabicyclo[3.2.1]octane-3-carboxylate (2.0 g, 1.0 equiv) was added. The reaction was stirred at 25° C. for 2 hours. The mixture was diluted with water (20 mL) and extracted with ethyl acetate (3×20 mL). The combined organic layers were dried over Na₂SO₄ and concentrated to afford the title compound (2.23 g, 86% yield) as yellow oil.

Step C. tert-butyl 1-(3-ethoxy-3-oxopropyl)-3-azabicyclo[3.2.1]octane-3-carboxylate: To a solution of tert-butyl(E)-1-(3-ethoxy-3-oxoprop-1-en-1-yl)-3-azabicyclo[3.2.1]octane-3-carboxylate (2.23 g, 1.0 equiv) in MeOH (20 mL) was added Pd/C (500 mg, 10% purity) under N₂ atmosphere. The suspension was degassed and purged with H₂ for 3 times. The reaction was stirred under H₂ (15 Psi) for 0.5 hours at 25° C. The mixture was filtered and the filtrate was concentrated in vacuum to afford the title compound (1.60 g, 71% yield) as colorless oil; ¹H NMR (400 MHZ, chloroform-d) δ=4.20-4.06 (m, 2H), 3.94-3.58 (m, 2H), 2.88-2.51 (m, 2H), 2.39-2.25 (m, 2H), 2.25-2.14 (m, 1H), 1.79-1.60 (m, 5H), 1.52 (br s, 1H), 1.46 (s, 9H), 1.39-1.30 (m, 2H), 1.26 (br t, J=7.2 Hz, 3H).

Step D. ethyl 3-(3-azabicyclo[3.2.1]octan-1-yl) propanoate: To a solution of tert-butyl 1-(3-ethoxy-3-oxopropyl)-3-azabicyclo[3.2.1]octane-3-carboxylate (200 mg, 1.0 equiv) in acetonitrile (1 mL) was added HCl·dioxane (4 M, 2.0 mL, 12 equiv) at 0° C. The reaction was stirred at 0° C. for 0.5 hours. The mixture was concentrated in vacuum to afford the title compound (150 mg, crude, HCl) as colorless oil.

Step E. ethyl 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoate: To a solution of 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (150 mg, 1.0 equiv), DIEA (152 mg, 5.0 equiv) and ethyl 3-(3-azabicyclo[3.2.1]octan-1-yl) propanoate (146 mg, 2.5 equiv, HCl) in DMF (0.5 mL) was added 4 Å molecular sieve (50 mg). The reaction was stirred at 40° C. for 12 hours. The mixture was filtered and purified by reversed phase flash chromatography [C18, 0.1% formic acid condition] to afford the title compound (110 mg, 62% yield) as a yellow solid; LCMS (ESI, M+1): m/z=748.4.

Step F. 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoic acid: To a solution of ethyl 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoate (110 mg, 1.0 equiv) in MeOH (1 mL) was added LiOH (2 M, 294 μL, 4.0 equiv). The reaction was stirred at 25° C. for 3 hours. The mixture was acidified with 1M HCl (0.5 mL) and extracted with ethyl acetate (3×1 mL). The combined organic layers were dried over Na₂SO₄ and concentrated to afford the title compound (110 mg, crude) as a yellow solid.

Step G. 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylpropanamide: To a mixture of 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoic acid (80 mg, 1.0 equiv) and methanamine hydrochloride (15 mg, 2.0 equiv) in DMF (1 mL) were added HATU (63.4 mg, 1.5 equiv) and DIEA (71.8 mg, 5.0 equiv). The reaction was stirred at 25° C. for 0.5 hours. The mixture was purified by reversed phase flash chromatography [C18, 0.1% formic acid condition] to afford the title compound (40 mg, 46% yield) as a yellow solid; LCMS (ESI, M+1): m/z=733.5.

Step H. 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylpropanamide: To a solution of 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylpropanamide (35 mg, 1.0 equiv) in MeCN (0.2 mL) was added HCl·dioxane (4 M, 0.4 mL, 33 equiv) at 0° C. The reaction was stirred at 0° C. for 0.5 hours. The mixture was concentrated, diluted with acetonitrile (1.0 ml), neutralized with solid NaHCO₃, concentrated and purified by prep-HPLC [column: Waters Xbridge 150×25 mm×5 μm; A: water (ammonia hydroxide v/v), B: ACN, B %: 37%-67% over 8 min] to afford the title compound (7.21 mg, 21% yield) as a white solid; ¹H NMR (400 MHZ, METHANOL-d₄) δ=9.05 (d, J=2.0 Hz, 1H), 7.65 (dd, J=5.6, 8.8 Hz, 1H), 7.28 (d, J=2.4 Hz, 1H), 7.23 (t, J=9.2 Hz, 1H), 7.05 (t, J=2.4 Hz, 1H), 5.39-5.22 (m, 1H), 4.69-4.57 (m, 2H), 4.37-4.19 (m, 2H), 3.71-3.61 (m, 1H), 3.44-3.34 (m, 2H), 3.29-3.16 (m, 3H), 3.07-2.95 (m, 1H), 2.72 (d, J=2.4 Hz, 3H), 2.54-2.42 (m, 2H), 2.35-2.22 (m, 3H), 2.21-2.10 (m, 2H), 2.05-1.95 (m, 2H), 1.94-1.73 (m, 5H), 1.70-1.43 (m, 4H), 0.85-0.75 (m, 3H); LCMS (ESI, M+1): m/z=689.5.

### Example 910

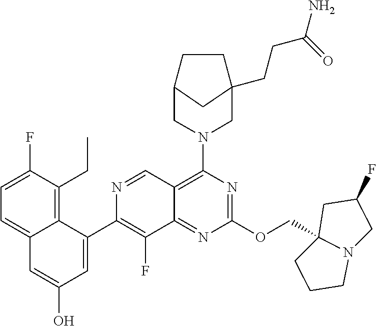 [SMILES: CCc1c(F)ccc2cc(O)cc(-c3ncc4c(N5CC6CCC(CCC(N)=O)(C6)C5)nc(OC[C@@]56CCCN5C[C@H](F)C6)nc4c3F)c12]

### 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)propanamide

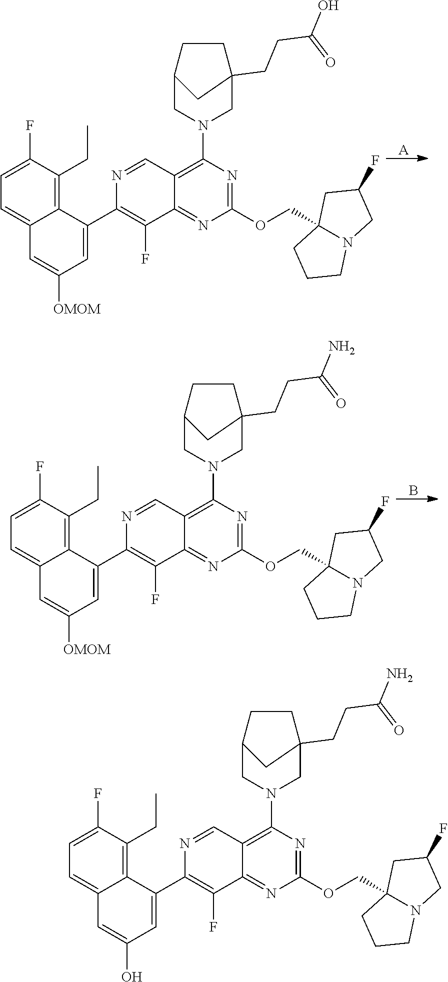

Step A. 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanamide: To a mixture of 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoic acid (50 mg, 1.0 equiv) in DMF (0.5 mL) were added NH₄Cl (7.43 mg, 2.0 equiv), HATU (39.6 mg, 1.5 equiv) and DIEA (44.9 mg, 5.0 equiv). The reaction was stirred at 25° C. for 0.5 hours. The mixture was purified by reversed phase flash chromatography [C18, 0.1% formic acid condition] to afford the title compound (20 mg, 37% yield) as yellow solid; LCMS (ESI, M+1): m/z=719.4.

Step B. 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanamide: To a solution of 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanamide (15 mg, 1.0 equiv) in acetonitrile (0.2 mL) was added HCl·dioxane (4.0 M, 306 μL, 59 equiv) at 0° C. The reaction was stirred at 0° C. for 0.5 hours. The mixture was concentrated, diluted with MeCN (1 ml) and neutralized with solid NaHCO₃. The mixture was filtered, concentrated and purified by prep-HPLC [column: Waters Xbridge 150×25 mm×5 μm; A: water (NH₄HCO₃), B: ACN, B %: 38%-68% over 9 min]. The desired fractions were collected and concentrated under vacuum to remove acetonitrile. The aqueous layers was lyophilized to afford the title compound (3.21 mg, 22% yield) as white solid; ¹H NMR (400 MHZ, METHANOL-d₄) δ=9.07 (d, J=2.4 Hz, 1H), 7.68 (dd, J=6.0, 9.2 Hz, 1H), 7.30 (d, J=2.4 Hz, 1H), 7.25 (t, J=9.6 Hz, 1H), 7.07 (t, J=2.8 Hz, 1H), 5.41-5.21 (m, 1H), 4.74-4.60 (m, 2H), 4.36-4.20 (m, 2H), 3.73-3.62 (m, 1H), 3.44-3.34 (m, 2H), 3.30-3.18 (m, 3H), 3.08-2.97 (m, 1H), 2.55-2.43 (m, 2H), 2.38-2.23 (m, 3H), 2.22-2.12 (m, 2H), 2.07-1.95 (m, 2H), 1.95-1.74 (m, 5H), 1.72-1.60 (m, 2H), 1.59-1.45 (m, 2H), 0.87-0.74 (m, 3H); LCMS (ESI, M+1): m/z=675.5.

### Example 911

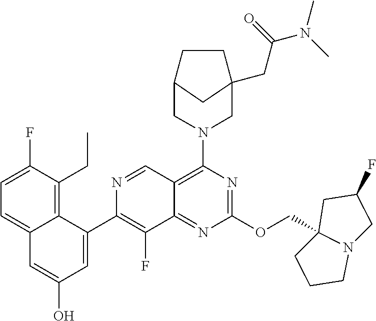 [SMILES: CCc1c(F)ccc2cc(O)cc(-c3ncc4c(N5CC6CCC(CC(=O)N(C)C)(C6)C5)nc(OC[C@@]56CCCN5C[C@H](F)C6)nc4c3F)c12]

### 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylacetamide

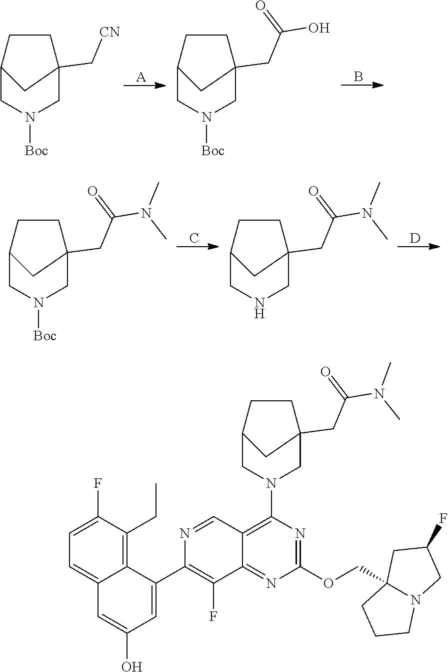

Step A. 2-(3-(tert-butoxycarbonyl)-3-azabicyclo[3.2.1]octan-1-yl) acetic acid: A mixture of tert-butyl 1-(cyanomethyl)-3-azabicyclo[3.2.1]octane-3-carboxylate (350 mg, 1.0 equiv), NaOH (10 M, 1.50 mL, 10.7 equiv) in EtOH (1.5 mL) was stirred at 80° C. for 48 hours under N₂ atmosphere. The mixture was adjusted to pH=5 with HCl (2 M), extracted with EtOAc (50 mL×3), washed with brine (50 mL), dried over Na₂SO₄, filtered and concentrated to afford the title compound (300 mg, crude) as a yellow oil. LCMS (ESI, M−1): m/z=268.0

Step B. tert-butyl 1-(2-(dimethylamino)-2-oxoethyl)-3-azabicyclo[3.2.1]octane-3-carboxylate: To a solution of 2-(3-(tert-butoxycarbonyl)-3-azabicyclo[3.2.1]octan-1-yl) acetic acid (40 mg, 1.0 equiv) and DIEA (86.4 mg, 4.50 equiv) in DMF (1.0 mL) was added HATU (169 mg, 3.0 equiv). The reaction mixture was stirred at 25° C. for 1 hour. N-methylmethanamine (2 M, 446 μL, 6.0 equiv) was added. The reaction was stirred at 25° C. for 12 hours. The mixture was extracted with EtOAc (20 mL×3), washed with brine (30 mL), dried over Na₂SO₄, filtered, concentrated and purified by reversed-phase HPLC (0.1% FA condition) to afford the title compound (30 mg, 62% yield) as a yellow oil. ¹H NMR (400 MHZ, CHLOROFORM-d) δ=3.92-3.71 (m, 2H), 3.03 (s, 3H), 2.94 (s, 3H), 2.87-2.72 (m, 2H), 2.50-2.33 (m, 2H), 2.20 (br d, J=18.8 Hz, 1H), 1.72-1.53 (m, 6H), 1.46 (s, 9H); LCMS (ESI, M+1): m/z=297.1.

Step C. 2-(3-azabicyclo[3.2.1]octan-1-yl)-N,N-dimethylacetamide: To a solution of tert-butyl 1-(2-(dimethylamino)-2-oxoethyl)-3-azabicyclo[3.2.1]octane-3-carboxylate (35 mg, 1.0 equiv) in MeOH (0.2 mL) was added HCl·MeOH (4.0 M, 13 equiv) at 0° C. The reaction was stirred at 0° C. for 0.5 hours. The mixture was concentrated in vacuum to afford the title compound (27.0 mg, crude, HCl) as yellow solid; LCMS (ESI, M+1): m/z=196.7.

Step D. 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylacetamide: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (30 mg, 1.0 equiv) and 2-(3-azabicyclo[3.2.1]octan-1-yl)-N,N-dimethylacetamide (23.6 mg, 2.0 equiv, HCl) in DMF (0.5 mL) were added DIEA (32.7 mg, 5.0 equiv) and 4 Å molecular sieve (10.0 mg, 1.0 equiv). The reaction was stirred at 40° C. for 12 hours. K₃PO₄ (21.5 mg, 2.0 equiv) was added. The reaction was stirred at 40° C. for 12 hours. The mixture was filtered and purified with prep-HPLC [column: Waters Xbridge 150×25 mm×5 um; A: water (NH₄HCO₃), B: ACN, B %: 48%-78% over 9 min] to afford the title compound (9.18 mg, 25% yield) as a white solid; ¹H NMR (400 MHZ, METHANOL-d₄) δ=9.09 (d, J=5.2 Hz, 1H), 7.67 (dd, J=6.0, 8.8 Hz, 1H), 7.30 (d, J=2.8 Hz, 1H), 7.24 (t, J=9.6 Hz, 1H), 7.07 (dd, J=2.4, 7.2 Hz, 1H), 5.44-5.20 (m, 1H), 4.73-4.62 (m, 2H), 4.41-4.18 (m, 2H), 3.67-3.56 (m, 1H), 3.56-3.44 (m, 1H), 3.27-3.15 (m, 3H), 3.11 (s, 3H), 3.06-2.98 (m, 1H), 2.93 (s, 3H), 2.73-2.54 (m, 2H), 2.52-2.41 (m, 2H), 2.25-2.10 (m, 3H), 2.05-1.96 (m, 2H), 1.95-1.79 (m, 4H), 1.78-1.70 (m, 1H), 1.65-1.50 (m, 2H), 1.36-1.26 (m, 1H), 0.86-0.75 (m, 3H); LCMS (ESI, M+1): m/z=689.4.

### Example 912

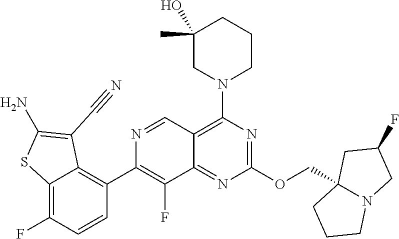 [SMILES: C[C@@]1(O)CCCN(c2nc(OC[C@@]34CCCN3C[C@H](F)C4)nc3c(F)c(-c4ccc(F)c5sc(N)c(C#N)c45)ncc23)C1]

### amino-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)pyrido[4,3-d]pyrimidin-7-yl)benzo[b]thiophene-3-

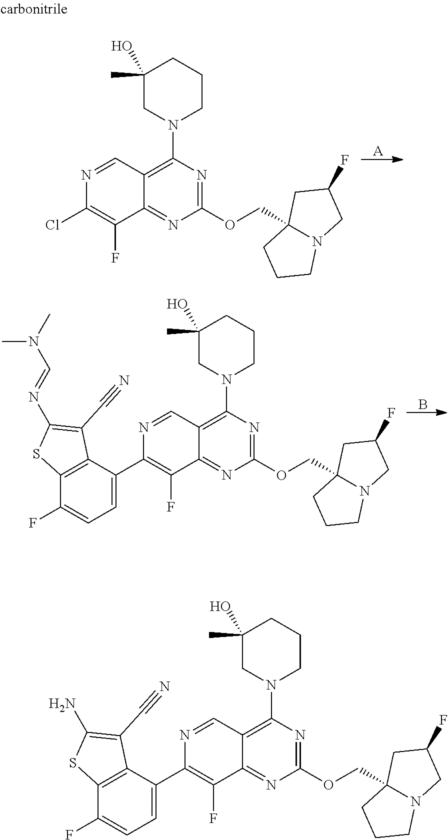

Step A. (E)-N′-(3-cyano-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)pyrido[4,3-d]pyrimidin-7-yl)benzo[b]thiophen-2-yl)-N,N-dimethylformimidamide: To a solution of (R)-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (160 mg, 1.0 equiv) and (E)-N′-(3-cyano-7-fluoro-4-(4,4,5,5-tetramethyl-1,3,2-dioxaborolan-2-yl)benzo[b]thiophen-2-yl)-N,N-dimethylformimidamide (150 mg, 1.2 equiv) in DMAc (4 mL) were added K₃PO₄ (1.5 M in H₂O, 660 μL, 3.0 equiv) and Brettphos Pd G4 (30.4 mg, 0.1 equiv). The reaction was degassed and purged with nitrogen 3 times. The reaction was stirred at 80° C. for 4.5 hours. The mixture was diluted with H₂O (10 mL) and extracted with EtOAc (4×10 mL). The combined organic layers were washed with brine (5 mL), dried over anhydrous sodium sulfate, concentrated, and purified with reversed phase flash chromatography [water (FA, 0.1%)/acetonitrile=3/2] to afford the title compound (40.0 mg, 18% yield) as light yellow solid; LCMS (ESI, M+1): m/z=665.2.

Step B 2-amino-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)pyrido[4,3-d]pyrimidin-7-yl)benzo[b]thiophene-3-carbonitrile: To a solution of (E)-N′-(3-cyano-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)pyrido[4,3-d]pyrimidin-7-yl)benzo[b]thiophen-2-yl)-N,N-dimethylformimidamide (40 mg, 1.0 equiv) in DMAc (1 mL) was added K₃PO₄ (1.5 M in H₂O, 1 mL). The reaction was stirred at 80° C. for 1 hour. The mixture was diluted with H₂O (6 mL) and extracted with EtOAc (4×3 mL). The combined organic layers were washed with brine (5 mL), dried over anhydrous sodium sulfate, concentrated and purified with prep-HPLC [column: Waters xbridge 150×25 mm×10 um; mobile phase: water (NH₄HCO₃)-ACN; B %: 37%-67% over 10 minutes] to afford the title compound (5.63 mg, 35% yield) as green solid; ¹H NMR (400 MHZ, METHANOL-d4) δ=9.20 (s, 1H), 7.41 (dd, J=5.2, 8.4 Hz, 1H), 7.05 (t, J=8.8 Hz, 1H), 5.38-5.23 (m, 1H), 4.51-4.47 (m, 1H), 4.34-4.22 (m, 3H), 3.65-3.61 (m, 1H), 3.49-3.39 (m, 1H), 3.28-3.17 (m, 3H), 3.05-2.95 (m, 1H), 2.25-2.12 (m, 4H), 2.01-1.74 (m, 6H), 1.27 (s, 3H); LCMS (ESI, M+1): m/z=610.2.

### Example 913

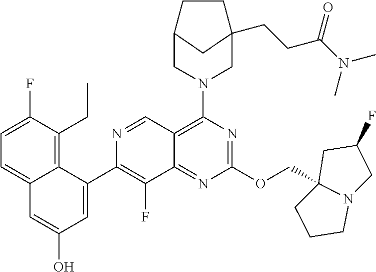 [SMILES: CCc1c(F)ccc2cc(O)cc(-c3ncc4c(N5CC6CCC(CCC(=O)N(C)C)(C6)C5)nc(OC[C@@]56CCCN5C[C@H](F)C6)nc4c3F)c12]

### 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylpropanamide

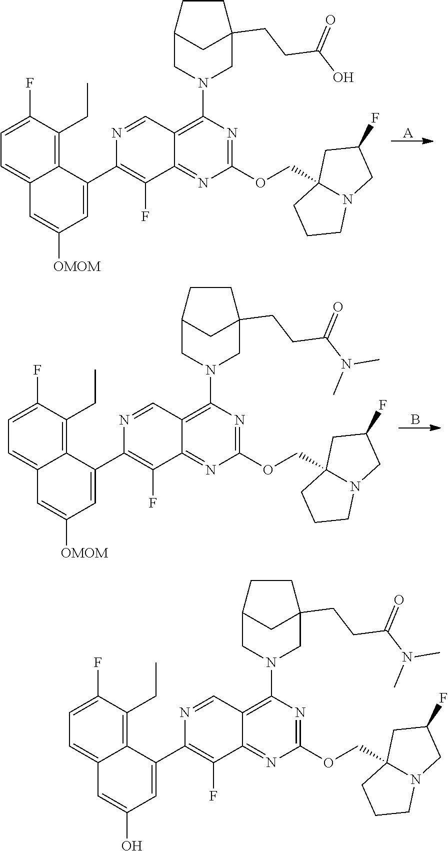

Step A. 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylpropanamide: To a solution of 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl) propanoic acid (60 mg, 1.0 equiv) and N-methylmethanamine (2.0 M, 2.0 equiv) in DMF (0.5 mL) was added HATU (47.5 mg, 1.5 equiv) and DIEA (32.3 mg, 3.0 equiv). The reaction was stirred at 25° C. for 0.5 hours. The mixture was purified by reversed phase flash chromatography [C18, 0.1% formic acid condition] to afford the title compound (27 mg, 43% yield) as a white solid; LCMS (ESI, M+1): m/z=747.8.

Step B. 3-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylpropanamide: To a solution of 3-(3-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N,N-dimethylpropanamide (22 mg, 1.0 equiv) in MeCN (0.2 mL) was added HCl·dioxane (4.0 M, 68 equiv) at 0° C. The reaction was stirred at 0° C. for 0.5 hours. The mixture was basified with saturated aqueous NaHCO₃ (0.5 mL) and extracted with ethyl acetate (3×3 mL). The combined organic layers were dried over anhydrous sodium sulfate, concentrated and purified by prep-HPLC [column: Waters Xbridge 150×25 mm×5 μm; A: water (NH₄HCO₃), B: ACN, B %: 45%-75% over 9 min] to afford the title compound (2.52 mg, 12% yield) as white solid; ¹H NMR (400 MHZ, METHANOL-d₄) δ=9.11-9.03 (m, 1H), 7.68 (dd, J=5.6, 9.2 Hz, 1H), 7.30 (d, J=2.4 Hz, 1H), 7.25 (t, J=9.2 Hz, 1H), 7.06 (d, J=2.8 Hz, 1H), 5.40-5.20 (m, 1H), 4.66 (br d, J=11.2 Hz, 2H), 4.35-4.22 (m, 2H), 3.71-3.62 (m, 1H), 3.50-3.38 (m, 2H), 3.25-3.14 (m, 3H), 3.10 (s, 3H), 3.04-2.99 (m, 1H), 2.95 (s, 3H), 2.55-2.43 (m, 4H), 2.28-2.12 (m, 3H), 2.04-1.96 (m, 2H), 1.92-1.76 (m, 5H), 1.71-1.50 (m, 4H), 0.86-0.75 (m, 3H); LCMS (ESI, M+1): m/z=703.4.

### Example 914

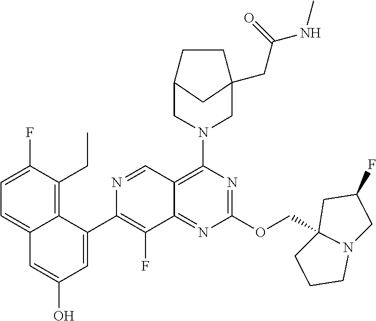 [SMILES: CCc1c(F)ccc2cc(O)cc(-c3ncc4c(N5CC6CCC(CC(=O)NC)(C6)C5)nc(OC[C@@]56CCCN5C[C@H](F)C6)nc4c3F)c12]

### 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylacetamide

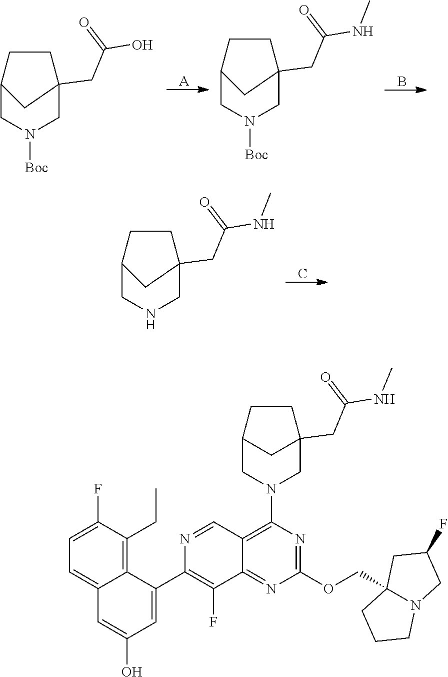

Step A. tert-butyl 1-(2-(methylamino)-2-oxoethyl)-3-azabicyclo[3.2.1]octane-3-carboxylate: To a solution of 2-(3-(tert-butoxycarbonyl)-3-azabicyclo[3.2.1]octan-1-yl) acetic acid (150 mg, 1.0 equiv) and methanamine;hydrochloride (75.2 mg, 2.0 equiv) in DMF (1.0 mL) were added DIEA (360 mg, 5.0 equiv) and HATU (318 mg, 1.5 equiv). The reaction was stirred at 25° C. for 0.5 hours. The mixture was quenched with water (3 mL) and extracted with ethyl acetate (3× 3 mL). The combined the organic layers were dried over anhydrous Na₂SO₄, concentrated and purified with reversed phase flash chromatography [water (0.1% FA)/acetonitrile] to afford the title compound (97 mg. 62% yield,) as a yellow solid. LCMS (ESI, M−1): m/z=283.3.

Step B. 2-(3-azabicyclo[3.2.1]octan-1-yl)-N-methylacetamide: To a solution of tert-butyl 1-(2-(methylamino)-2-oxoethyl)-3-azabicyclo[3.2.1]octane-3-carboxylate (80 mg, 1.0 equiv) in MeOH (0.5 mL) was added HCl/MeOH (4.0 M, 14 equiv) at 0° C. The reaction was stirred at 25° C. for 0.5 hours. The reaction was concentrated to afford the title compound (61 mg, crude, HCl) as a yellow solid; LCMS (ESI, M+1): m/z=182.7.

Step C. 2-(3-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-1-yl)-N-methylacetamide: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (80 mg, 1.0 equiv) and 2-(3-azabicyclo[3.2.1]octan-1-yl)-N-methylacetamide (51 mg, 1.7 equiv, HCl) in DMF (1 mL) was added 4 Å molecular sieve (40 mg, 1.0 equiv) and K₃PO₄ (143 mg, 5.0 equiv). The reaction was stirred at 40° C. for 12 hours. The mixture was filtered and purified by prep-HPLC [column: Waters Xbridge 150×25 mm×5 μm; A: water (NH₄HCO₃), B: ACN, B %: 38%-68% over 9 min] to afford the title compound (9.45 mg, 10% yield) as a white solid; ¹H NMR (400 MHZ, METHANOL-d₄) δ=9.07 (s, 1H), 7.67 (dd, J=5.6, 8.8 Hz, 1H), 7.34-7.19 (m, 2H), 7.06 (dd, J=2.4, 5.2 Hz, 1H), 5.44-5.20 (m, 1H), 5.09-4.95 (m, 2H), 4.38-4.21 (m, 2H), 3.69-3.56 (m, 1H), 3.47-3.39 (m, 1H), 3.30-3.12 (m, 3H), 3.09-2.97 (m, 1H), 2.73 (s, 3H), 2.55-2.44 (m, 2H), 2.43-2.33 (m, 2H), 2.33-2.11 (m, 4H), 2.06-1.96 (m, 2H), 1.95-1.79 (m, 3H), 1.77-1.67 (m, 2H), 1.66-1.49 (m, 2H), 0.89-0.71 (m, 3H); LCMS (ESI, M+1): m/z=675.5.

### Example 915

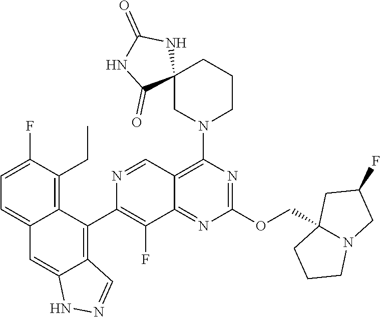 [SMILES: CCc1c(F)ccc2cc3[nH]ncc3c(-c3ncc4c(N5CCC[C@]6(C5)NC(=O)NC6=O)nc(OC[C@@]56CCCN5C[C@H](F)C6)nc4c3F)c12]

### (5R)-7-(7-(5-ethyl-6-fluoro-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione

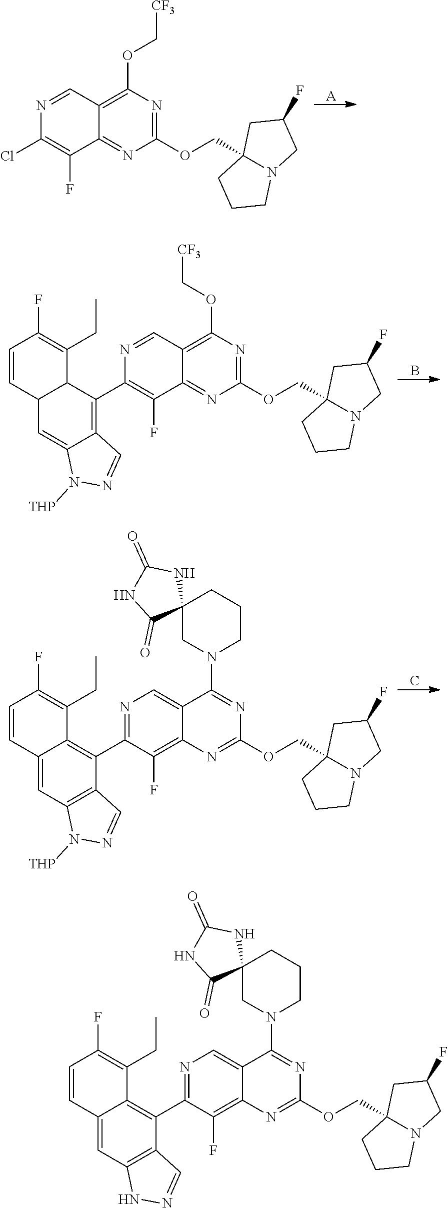

Step A. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-1-(tetrahydro-2H-pyran-2-yl)-1H-benzo[f]indazole: To a solution of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (300 mg, 1.0 equiv), 5-ethyl-6-fluoro-1-(tetrahydro-2H-pyran-2-yl)-4-(4,4,5,5-tetramethyl-1,3,2-dioxaborolan-2-yl)-1H-benzo[f]indazole (348 mg, 1.2 equiv) and K₃PO₄ (1.5 M in water, 3.0 equiv) in toluene (7 mL) was added and Aphos Pd G3 (45.2 mg, 0.1 equiv). The reaction was degassed and purged with nitrogen 3 times. The reaction was stirred at 70° C. for 16 hours. The mixture was diluted with H₂O (10 mL) and extracted with ethyl acetate (2×15 mL). The combined organic layers were washed with brine (10 mL), dried over anhydrous sodium sulfate, concentrated, and purified with reversed phase flash chromatography [C18, 0.1% formic acid condition] to afford the title compound (160 mg, 29% yield) as a yellow solid; LCMS (ESI, M+1): m/z=701.2.

Step B. (5R)-7-(7-(5-ethyl-6-fluoro-1-(tetrahydro-2H-pyran-2-yl)-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-1-(tetrahydro-2H-pyran-2-yl)-1H-benzo[f]indazole (150 mg, 1.0 equiv) and (R)-1,3,7-triazaspiro[4.5]decane-2,4-dione (72.0 mg, 2 equiv) in DMF (0.5 mL) was added DIEA (55.3 mg, 2.0 equiv). The reaction was stirred at 40° C. for 12 hours. The mixture was diluted with H₂O (10 mL) and extracted with ethyl acetate (2×15 mL). The combined organic layers were washed with brine (3×5 mL), dried over anhydrous sodium sulfate, concentrated and purified with reversed phase flash chromatography [C18, 0.1% formic acid condition] to afford the title compound (100 mg, 61% yield) a yellow oil; LCMS (ESI, M+1): m/z=770.3.

Step C. (5R)-7-(7-(5-ethyl-6-fluoro-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione: To a solution of (5R)-7-(7-(5-ethyl-6-fluoro-1-(tetrahydro-2H-pyran-2-yl)-1H-benzo[f]indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione (90.0 mg, 1.0 equiv) in DCM (2 mL) was added TFA (4 ml) at 0° C. The reaction was stirred at 20° C. for 0.5 hours. The mixture was diluted with NaHCO₃ aqueous (10 mL) and extracted with ethyl acetate (2×15 mL). The combined organic layer was concentrated and purified with prep-HPLC [column: Phenomenex luna C18 150×25 mm×10 μm; A: water (FA), B: ACN, B %: 11%-41% over 10 min] and lyophilized to afford the title compound (17.9 mg, 21% yield, 0.45 HCOOH) as yellow solid; ¹H NMR (400 MHZ, METHANOL-d4) δ=9.25 (s, 1H), 8.26 (s, 1H), 8.02 (dd, J=6.0, 9.2 Hz, 1H), 7.70 (d, J=7.2 Hz, 1H), 7.34 (t, J=9.6 Hz, 1H), 5.53-5.32 (m, 1H), 4.73-4.49 (m, 3H), 4.48-4.40 (m, 2H), 3.93-3.74 (m, 2H), 3.66-3.45 (m, 3H), 3.25-3.16 (m, 1H), 2.73-2.53 (m, 1H), 2.46-2.34 (m, 2H), 2.32-2.22 (m, 2H), 2.19-1.93 (m, 6H), 0.86 (q, J=7.2 Hz, 3H); LCMS (ESI, M+1): m/z=686.2.

### Example 916

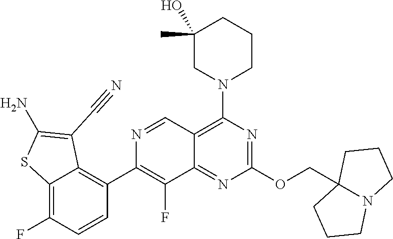 [SMILES: C[C@@]1(O)CCCN(c2nc(OCC34CCCN3CCC4)nc3c(F)c(-c4ccc(F)c5sc(N)c(C#N)c45)ncc23)C1]

### (R)-2-amino-7-fluoro-4-(8-fluoro-4-(3-hydroxy-3-methylpiperidin-1-yl)-2-((tetrahydro-1H-pyrrolizin-7a(5H)-yl)methoxy)pyrido[4,3-d]pyrimidin-7-yl)benzo[b]thiophene-3-carbonitrile

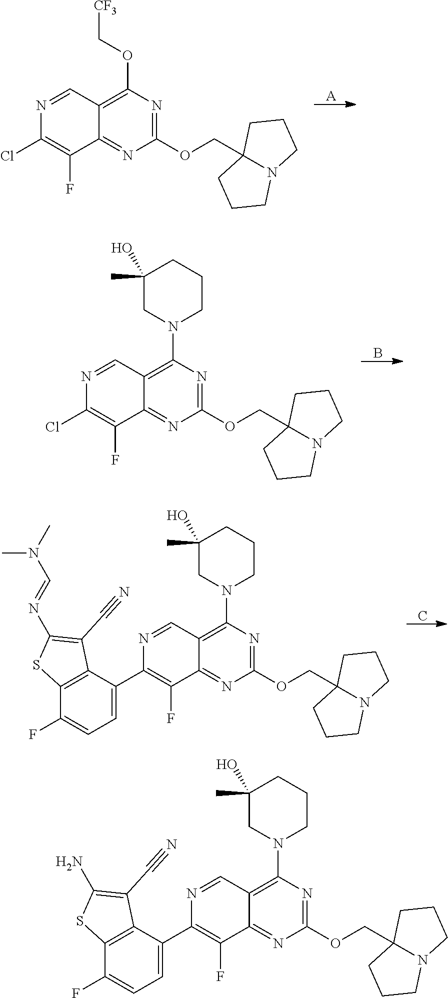

Step A. (R)-1-(7-chloro-8-fluoro-2-((tetrahydro-1H-pyrrolizin-7a(5H)-yl)methoxy)pyrido[4,3-d]pyrimidin-4-yl)-3-methylpiperidin-3-ol: To a solution of 7-chloro-8-fluoro-2-((tetrahydro-1H-pyrrolizin-7a(5H)-yl)methoxy)-4-(2,2,2-trifluoroethoxy)pyrido[4,3-d]pyrimidine (350 mg, 1.0 equiv) and DIEA (215 mg, 2.0 equiv) in DMF (3 mL) was added (3R)-3-methylpiperidin-3-ol (144 mg, 1.5 equiv) at 40° C. The reaction was stirred at 40° C. for 14 hours. The mixture was diluted with H₂O (20 mL) and extracted with EtOAc (3×10 mL). The combined organic layers were washed with brine (5 mL), dried over anhydrous sodium sulfate, concentrated, and purified with reversed phase flash chromatography [C18, 0.1% formic acid condition] to afford the title compound (180 mg, 48% yield) as light yellow solid; LCMS (ESI, M+1): m/z=436.2.

Step B. (R,E)-N′-(3-cyano-7-fluoro-4-(8-fluoro-4-(3-hydroxy-3-methylpiperidin-1-yl)-2-((tetrahydro-1H-pyrrolizin-7a(5H)-yl)methoxy)pyrido[4,3-d]pyrimidin-7-yl)benzo[b]thiophen-2-yl)-N,N-dimethylformimidamide: To a solution of (R)-1-(7-chloro-8-fluoro-2-((tetrahydro-1H-pyrrolizin-7a(5H)-yl)methoxy)pyrido[4,3-d]pyrimidin-4-yl)-3-methylpiperidin-3-ol (160 mg, 1.0 equiv) and (E)-N′-(3-cyano-7-fluoro-4-(4,4,5,5-tetramethyl-1,3,2-dioxaborolan-2-yl)benzo[b]thiophen-2-yl)-N,N-dimethylformimidamide (160 mg, 1.2 equiv) in DMAc (4 mL) were added K₃PO₄ (1.5 M in H₂O, 3.0 equiv) and Brettphos Pd G4 (33.8 mg, 0.1 equiv). The reaction was degassed and purged with nitrogen 3 times. The reaction was stirred at 80° C. for 4.5 hours. The mixture was diluted with H₂O (10 mL) and extracted with EtOAc (4×10 mL). The combined organic layers were washed with brine (5 mL), dried over anhydrous sodium sulfate, concentrated, and purified with reversed phase flash chromatography [C18, 0.1% formic acid condition] to afford the title compound (160 mg, 55% yield) as brown solid; LCMS (ESI, M+1): m/z=647.3.

Step C. (R)-2-amino-7-fluoro-4-(8-fluoro-4-(3-hydroxy-3-methylpiperidin-1-yl)-2-((tetrahydro-1H-pyrrolizin-7a(5H)-yl)methoxy)pyrido[4,3-d]pyrimidin-7-yl)benzo[b]thiophene-3-carbonitrile: To a solution of (R,E)-N′-(3-cyano-7-fluoro-4-(8-fluoro-4-(3-hydroxy-3-methylpiperidin-1-yl)-2-((tetrahydro-1H-pyrrolizin-7a(5H)-yl)methoxy)pyrido[4,3-d]pyrimidin-7-yl)benzo[b]thiophen-2-yl)-N,N-dimethylformimidamide (150 mg, 1.0 equiv) in DMAc (2 mL) was added K₃PO₄ (1.5 M in H₂O, 1.50 mL). The reaction was stirred at 80° C. for 0.5 hours. The mixture was diluted with H₂O (6 mL) and extracted with EtOAc (4×5 mL). The combined organic layers were washed with brine (5 mL), dried over anhydrous sodium sulfate, concentrated, and purified with prep-HPLC [column: Phenomenex luna C18 150× 25 mm×10 μm; A: water (FA), B: ACN, B %: 11%-41% over 10 minutes] to afford the title compound (50.1 mg, 35% yield, 0.88 HCOOH) as yellow solid; ¹H NMR (400 MHZ, METHANOL-d4) δ=9.27 (s, 1H), 7.43 (dd, J=5.2, 8.4 Hz, 1H), 7.07 (t, J=8.8 Hz, 1H), 4.64-4.60 (s, 2H), 4.60-4.56 (m, 1H), 4.34-4.30 (m, 1H), 3.70-3.58 (m, 3H), 3.49-3.38 (m, 1H), 3.28-3.17 (m, 2H), 2.33-2.29 (m, 2H), 2.25-2.01 (m, 7H), 1.92-1.71 (m, 3H), 1.29 (s, 3H); LCMS (ESI, M+1): m/z=592.2.

### Example 917

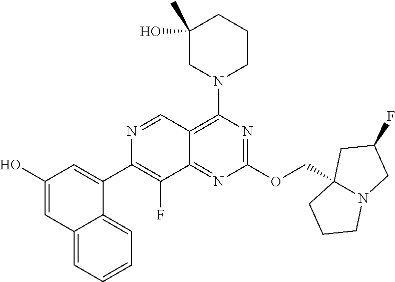 [SMILES: C[C@@]1(O)CCCN(c2nc(OC[C@@]34CCCN3C[C@H](F)C4)nc3c(F)c(-c4cc(O)cc5ccccc45)ncc23)C1]

### (R)-1-(8-fluoro-2-(((2R,7aS)-2-fluorotetrahydro-1H-pyrrolizin-7a(5H)-yl)methoxy)-7-(3-hydroxynaphthalen-1-yl)pyrido[4,3-d]pyrimidin-4-yl)-3-methylpiperidin-3-ol

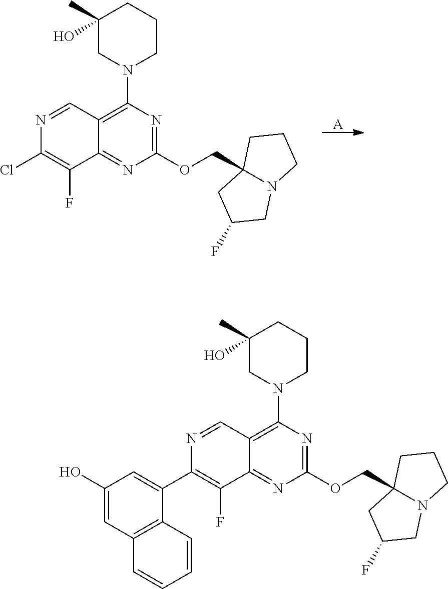

Step A. (R)-1-(8-fluoro-2-(((2R,7aS)-2-fluorotetrahydro-1H-pyrrolizin-7a(5H)-yl)methoxy)-7-(3-hydroxynaphthalen-1-yl)pyrido[4,3-d]pyrimidin-4-yl)-3-methylpiperidin-3-ol: To a suspension of (R)-1-(7-chloro-8-fluoro-2-(((2R,7aS)-2-fluorotetrahydro-1H-pyrrolizin-7a(5H)-yl)methoxy)pyrido[4,3-d]pyrimidin-4-yl)-3-methylpiperidin-3-ol (700 mg, 1.0 equiv), 4-(4,4,5,5-tetramethyl-1,3,2-dioxaborolan-2-yl)naphthalen-2-ol (500 mg, 1.2 equiv) and K₃PO₄ (1.5 M, 3.08 mL, 3.0 equiv) in methoxycyclopentane (10 mL) was added AdanBuP Pd G3 (196 mg, 0.20 equiv). The reaction was degassed and purged with nitrogen for 3 times. The reaction was stirred at 70° C. for 16 hours. The mixture was diluted with water (10 mL) and extracted with dichloromethane (4×15 mL). The combined organic layers were washed with brine (3×10 mL), dried over anhydrous sodium sulfate, concentrated and purified with prep-HPLC [Waters Xbridge Prep OBD C18 150×40 mm×10 um; mobile phase: A: water (NH₄HCO₃), B: ACN, 25%-55% over 10 min] and prep-HPLC [Phenomenex luna C18 150×40 mm×15 μm, A: water (FA), B: ACN, 12%-42% over 15 min] to afford the title compound (130 mg, 14% yield) as yellow solid; ¹H NMR (400 MHZ, DMSO) ¿=9.30 (s, 1H), 8.18 (s, 1H), 7.82-7.80 (m, J=8.4 Hz, 1H), 7.57-7.54 (m, J=12.0 Hz, 1H), 7.45 (m, 1H), 7.30-7.23 (m, 3H), 5.36-5.22 (d, 1H), 4.39-4.35 (d, 1H), 4.17-4.04 (m, 3H), 3.62-2.75 (m, 6H), 2.07 (m, J=2.0 Hz, 1H), 2.02 (m, 3H), 1.78-1.66 (m, 6H), 1.18 (s, 3H); LCMS (ESI, M+1): m/z=562.4.

### Example 918

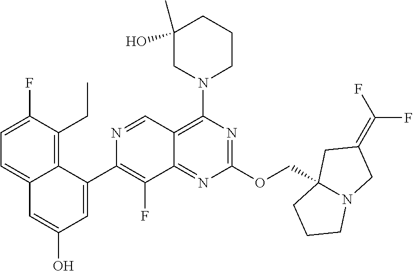 [SMILES: CCc1c(F)ccc2cc(O)cc(-c3ncc4c(N5CCC[C@@](C)(O)C5)nc(OC[C@@]56CCCN5CC(=C(F)F)C6)nc4c3F)c12]

### (R)-1-(2-(((S)-2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

### Example 919

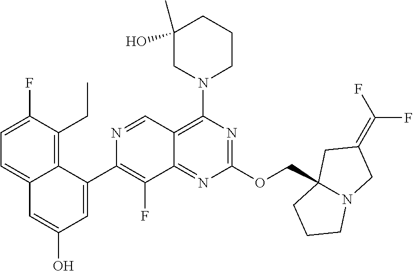 [SMILES: CCc1c(F)ccc2cc(O)cc(-c3ncc4c(N5CCC[C@@](C)(O)C5)nc(OC[C@]56CCCN5CC(=C(F)F)C6)nc4c3F)c12]

### (R)-1-(2-(((R)-2-(difluoromethylene)tetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-methylpiperidin-3-ol

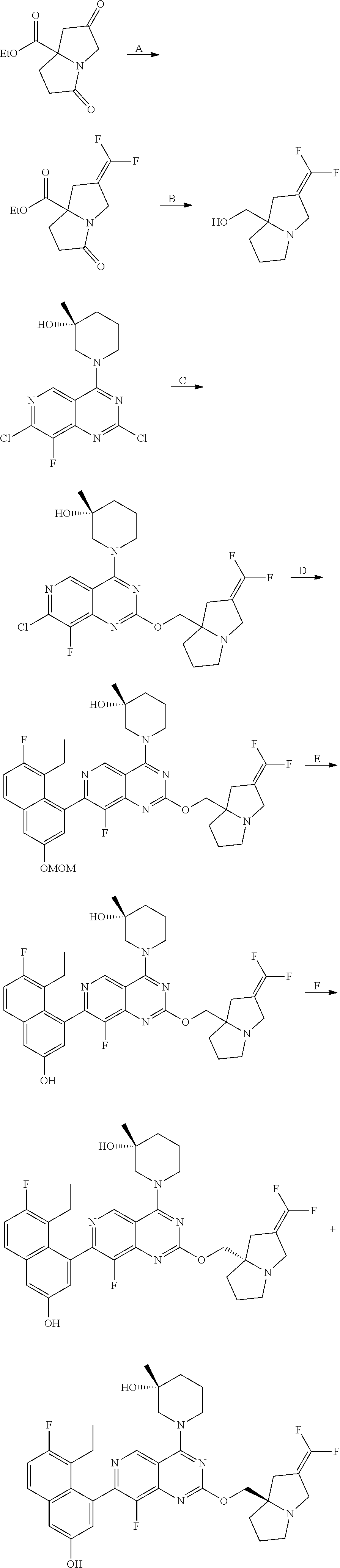

Step A: ethyl 2-(difluoromethylene)-5-oxohexahydro-1H-pyrrolizine-7a-carboxylate: To a mixture of ethyl 2,5-dioxohexahydro-1H-pyrrolizine-7a-carboxylate and 2-((difluoromethyl) sulfonyl)pyridine (7.13 g, 1.3 equiv) in DMF (60 mL) was added a solution of t-BuOK (5.74 g, 1.8 equiv) in DMF (10 mL) at −40° C. under N₂. The reaction was stirred at −40° C. for 2 hours and 20° C. for 1 hour. The mixture was quenched with NH₄Cl (50 mL) and 3M HCl (60 mL). The reaction was stirred at 20° C. for 30 minutes. The mixture was extracted with ethyl acetate (3×20 mL). The combined organic phase was washed with brine (20 mL), dried over anhydrous sodium sulfate, concentrated, and purified with silica gel chromatography and reversed phase flash chromatography to afford the title compound (1.30 g, 44% yield) as yellow oil, ¹H NMR (400 MHZ, DMSO-d₆) δ=4.35-4.31 (m, 1H), 4.24-4.18 (m, 2H), 3.74 (d, J=14.4 Hz, 1H), 3.13 (d, J=15.6 Hz, 1H), 2.81-2.72 (m, 1H), 2.63 (t, J=12.0 Hz, 1H), 2.48-2.36 (m, 2H), 2.71-2.08 (m, 1H), 1.29-1.25 (m, 3H).

Step B: (2-(difluoromethylene) hexahydro-1H-pyrrolizin-7a-yl)methanol: To a solution of ethyl 2-(difluoromethylene)-5-oxohexahydro-1H-pyrrolizine-7a-carboxylate (1.00 g, 1.0 equiv) in THF (10 mL) was added DIBAL-H (1 M, 10.0 equiv) at −70° C. The reaction was stirred at −70° C. for 1 hour. The mixture was quenched with Na₂SO₄·10H₂O at 0° C. and stirred at 20° C. for 1 hour. The mixture was filtered, concentrated and purified by silica gel chromatography to afford the title compound (300 mg, 39% yield) as yellow oil. ¹H NMR (400 MHz, DMSO-d₆) δ=3.69 (d, J=12.0 Hz, 1H), 3.40-3.29 (m, 3H), 3.15-3.13 (m, 1H), 2.67-2.64 (m, 1H), 2.49 (d, J=12.0 Hz, 1H), 2.38 (d, J=12.0 Hz, 1H), 2.00-1.95 (m, 1H), 1.89-1.84 (m, 2H), 1.71-1.76 (m, 1H).

Step C. (3R)-1-(7-chloro-2-((2-(difluoromethylene) hexahydro-1H-pyrrolizin-7a-yl)methoxy)-8-fluoropyrido[4,3-d]pyrimidin-4-yl)-3-methylpiperidin-3-ol: A mixture of (R)-1-(2,7-dichloro-8-fluoropyrido[4,3-d]pyrimidin-4-yl)-3-methylpiperidin-3-ol (200 mg, 1.00 equiv) and (2-(difluoromethylene) hexahydro-1H-pyrrolizin-7a-yl)methanol (114 mg, 1.0 equiv) in THF (4 mL) was added NaHMDS (1 M, 1.25 equiv) dropwise at −10° C. The reaction was stirred at −10° C. for 3 hours. The mixture was quenched with NH₄Cl (5 mL) and extracted with ethyl acetate (2× 5 mL). The combined organic layers were washed with brine (5 mL), dried over anhydrous sodium sulfate, concentrated, and purified with silica gel chromatography to afford the title compound (130 mg, 45% yield) as yellow solid; LCMS (ESI, M+1): m/z=484.3.

Step D. (3R)-1-(2-((2-(difluoromethylene) hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol: To a mixture of (3R)-1-(7-chloro-2-((2-(difluoromethylene) hexahydro-1H-pyrrolizin-7a-yl)methoxy)-8-fluoropyrido[4,3-d]pyrimidin-4-yl)-3-methylpiperidin-3-ol (130 mg, 1.0 equiv), 2-(8-ethyl-7-fluoro-3-(methoxymethoxy)naphthalen-1-yl)-4,4,5,5-tetramethyl-1,3,2-dioxaborolane (116 mg, 1.2 equiv) and K₃PO₄ (171 mg, 3.0 equiv) in dioxane (2 mL) and H₂O (0.5 mL) was added Pd(dtbpf) C12 (17.5 mg, 0.1 equiv). The reaction was degassed and purged with nitrogen 3 times. The reaction was stirred at 100° C. for 2 hrs. The mixture was diluted with ice-water (5 mL) and extracted with ethyl acetate (5 mL×2). The combined organic phase was washed with brine (3 mL), dried over anhydrous sodium sulfate, concentrated, and purified with silica gel chromatography to afford the title compound (120 mg, 66% yield) as yellow solid; LCMS (ESI, M+1): m/z=682.6.

Step E. (3R)-1-(2-((2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: To a mixture of compound (3R)-1-(2-((2-(difluoromethylene) hexahydro-1H-pyrrolizin-7a-yl)methoxy)-7-(8-ethyl-7-fluoro-3-(methoxymethoxy)naphthalen-1-yl)-8-fluoropyrido[4,3-d]pyrimidin-4-yl)-3-methylpiperidin-3-ol (110 mg, 1.0 equiv) in MeCN (1 mL) was added HCl·dioxane (4 M, 12.4 equiv). The reaction was stirred at 20° C. for 1 hr. The mixture was concentrated and purified by prep-HPLC [column: Welch Xtimate C18 150*25 mm*5 um; mobile phase: [water (HCl)-ACN]; gradient: 22%-52% B over 8 min] to afford the title compound (45.6 mg, 44% yield, HCl) as yellow solid; ¹H NMR (400 MHz, METHANOL-d₄) δ=9.63 (s, 1H), 7.77-7.74 (m, 1H), 7.42 (d, J=2.40 Hz, 1H), 7.33 (t, J=9.20 Hz, 1H), 7.21 (d, J=2.40 Hz, 1H), 5.00-4.94 (m, 2H), 4.63-4.57 (m, 1H), 4.49-4.45 (m, 1H), 4.17-4.14 (d, J=14.0 Hz, 1H), 3.74-3.70 (m, 1H), 3.51-3.50 (m, 1H), 3.41-3.35 (m, 1H), 3.17 (d, J=12.0 Hz, 1H), 2.99 (d, J=16.0 Hz, 1H), 2.51-2.19 (m, 8H), 1.92-1.90 (m, 3H), 1.40 (s, 3H), 0.93-0.88 (m, 3H); LCMS (ESI, M+1): m/z=638.3.

Step F. (R)-1-(2-(((S)-2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol and (R)-1-(2-(((R)-2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol: (3R)-1-(2-((2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol (40 mg) was separated by SFC [column: Chiralpak IG-3 50*4.6 mm I.D., 3 um Mobile phase: Phase A for CO₂, and Phase B for EtOH (0.05% DEA); Gradient elution: 30% EtOH (0.05% DEA) in CO₂, Flow rate: 3 mL/min; Detector: PDA; Colum Temp: 35C; Back Pressure: 100Bar] to give two peaks

Peak 1 Example 918: (R)-1-(2-(((S)-2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol (peak1) (12.0 mg, 30% yield) ¹H NMR (400 MHZ, METHANOL-d₄) δ=9.11 (s, 1H), 7.59-7.55 (m, 1H), 7.19 (d, J=4.00 Hz, 1H), 7.14 (t, J=12.0 Hz, 1H), 6.95 (s, 1H), 4.50-4.42 (m, 1H), 4.31-4.28 (m, 1H), 4.22-4.16 (m, 2H), 3.68 (d, J=24.0 Hz, 1H), 3.57-3.47 (m, 1H), 3.39-3.32 (m, 1H), 3.07-3.03 (m, 1H), 2.69 (d, J=16.0 Hz, 1H), 2.64-2.58 (m, 1H), 2.43-2.35 (m, 2H), 2.13-2.02 (m, 3H), 1.92-1.89 (m, 1H), 1.85-1.78 (m, 2H), 1.77-175 (m, 2H); 1.72-1.66 (m, 2H), 1.20-1.17 (m, 3H), 0.74-0.69 (m, 3H); LCMS (ESI, M+1): m/z=638.4; SFC: Rt=1.338

Peak 2 Example 919: (R)-1-(2-(((R)-2-(difluoromethylene) hexahydro-1H-pyrrolizin-7a-yl)methoxy)-7-(8-ethyl-7-fluoro-3-hydroxynaphthalen-1-yl)-8-fluoropyrido[4,3-d]pyrimidin-4-yl)-3-methylpiperidin-3-ol (8.20 mg, 20% yield); ¹H NMR (400 MHZ, METHANOL-d₄) δ=9.24 (s, 1H), 7.72-7.68 (m, 1H), 7.33 (d, J=4.00 Hz, 1H), 7.27 (t, J=8.00 Hz, 1H), 7.08 (s, 1H), 4.59-4.55 (m, 1H), 4.49 (d, J=12.0 Hz, 1H), 4.40-4.29 (m, 2H), 3.96-3.92 (m, 2H), 3.69-3.62 (m, 1H), 3.55-3.45 (m, 2H), 3.28-3.25 (m, 1H), 2.89-2.83 (m, 2H), 2.62 (d, J=12.0 Hz, 1H), 2.52-2.47 (m, 1H), 2.25-2.16 (m, 3H), 2.08-1.97 (m, 3H), 1.91-1.78 (m, 3H), 1.33 (d, J=8.00 Hz, 3H); 0.86-0.80 (m, 3H); LCMS (ESI, M+1): m/z=638.4; SFC: Rt=1.815

### Example 920

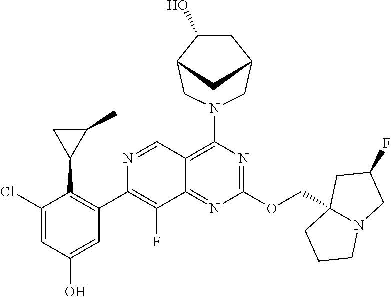 [SMILES: C[C@@H]1C[C@@H]1c1c(Cl)cc(O)cc1-c1ncc2c(N3C[C@@H]4C[C@H](C3)[C@H](O)C4)nc(OC[C@@]34CCCN3C[C@H](F)C4)nc2c1F]

### (1R,5R,6R)-3-(7-(3-chloro-5-hydroxy-2-(cis-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol

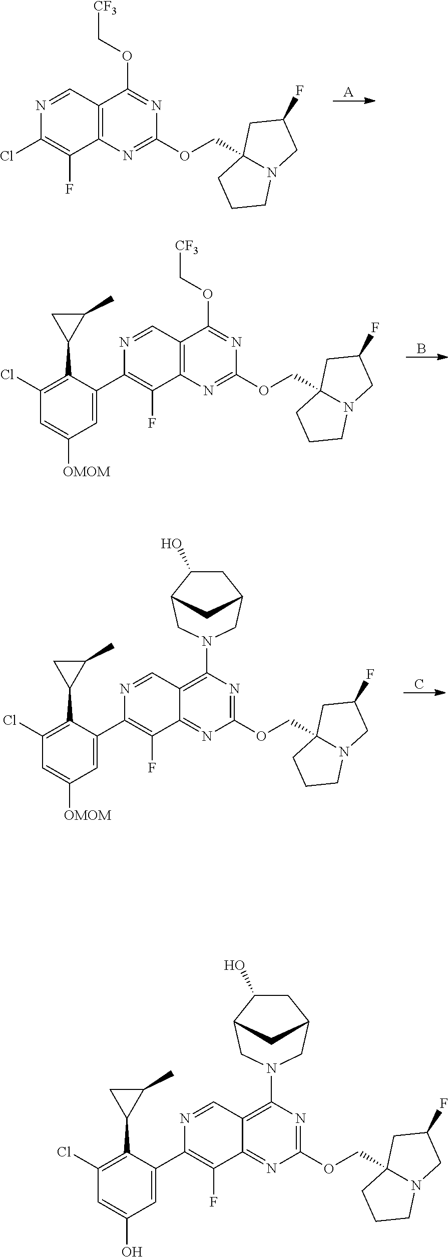

Step A. 7-(3-chloro-5-(methoxymethoxy)-2-(cis-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a mixture of 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (450 mg, 1.0 equiv) and 2-(3-chloro-5-(methoxymethoxy)-2-(cis-2-methylcyclopropyl)phenyl)-4,4,5,5-tetramethyl-1,3,2-dioxaborolane (362 mg, 1.0 equiv) in methoxycyclopentane (5 mL) and water (1.3 mL) were added CataCXium A Pd G3 (149 mg, 0.2 equiv) and Cs₂CO₃ (1.00 g, 3.0 equiv). The reaction was degassed and purged with N₂ for 3 times. The reaction was stirred at 90° C. for 10 hours under N₂ atmosphere. The mixture was diluted with water (50 mL) and extracted with EtOAc (2×50 mL). The combined organic layers were washed with brine (40 mL), dried over anhydrous sodium sulfate, concentrated and purified with column chromatography [SiO₂, petroleum ether/ethyl acetate=10/1 to 0/1] to afford the title compound (320 mg, 40% yield) as yellow solid; LCMS (ESI, M+1): m/z=629.2.

Step B. (1R,5R,6R)-3-(7-(3-chloro-5-(methoxymethoxy)-2-(cis-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of 7-(3-chloro-5-(methoxymethoxy)-2-(cis-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (320 mg, 1.0 equiv) in DMF (6 mL) were added (1R,5R,6R)-3-azabicyclo[3.2.1]octan-6-ol (104 mg, 2.0 equiv), DIEA (105 mg, 2.0 equiv) and 4 Å molecular sieve (50.0 mg). The reaction was stirred at 40° C. for 24 hours. The mixture was diluted with water (30 mL) and extracted with EtOAc (3×30 mL). The combined organic layers were washed with brine (80 mL), dried over anhydrous sodium sulfate, concentrated and purified with column chromatography [SiO₂, ethyl acetate/ethanol=1/0 to 1/1] to afford the title compound (277 mg, 77% yield) as yellow solid; LCMS (ESI, M+1): m/z=656.4.

Step C. (1R,5R,6R)-3-(7-(3-chloro-5-hydroxy-2-(cis-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(3-chloro-5-(methoxymethoxy)-2-(cis-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (220 mg, 1.0 equiv) in acetonitrile (2 mL) was added HCl·dioxane (4 M, 2 mL) at 0° C. The reaction was stirred at 0° C. for 3 hours. The mixture was concentrated, dissolved in MeOH (2 mL), adjusted to pH=7 with solid NaHCO₃ and filtered. The filtrate was concentrated and purified with prep-HPLC [neutral condition; column: Waters Xbridge 150×25 mm×5 μm; mobile phase: water (NH₄HCO₃)-ACN; gradient: 40%-70% B over 9 min] to afford the title compound (73.8 mg, 48% yield) as white solid; 1HNMR (400 MHZ, DMSO-d₆) δ=9.97 (s, 1H), 9.35-9.25 (m, 1H), 6.98 (d, J=2.4 Hz, 1H), 6.85-6.77 (m, 1H), 5.42-5.15 (m, 1H), 4.91-4.47 (m, 3H), 4.24-4.07 (m, 2H), 3.99 (dd, J=6.4, 10.0 Hz, 1H), 3.83-3.58 (m, 1H), 3.29 (d, J=12.4 Hz, 1H), 3.15-2.97 (m, 3H), 2.88-2.76 (m, 1H), 2.32 (s, 1H), 2.16-1.98 (m, 5H), 1.90-1.63 (m, 6H), 1.23 (d, J=13.2 Hz, 1H), 1.14-0.92 (m, 1H), 0.55 (s, 4H); LCMS (ESI, M+1): m/z=612.3.

### Example 921

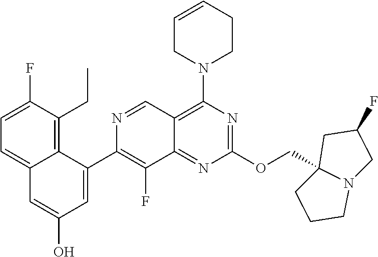 [SMILES: CCc1c(F)ccc2cc(O)cc(-c3ncc4c(N5CC=CCC5)nc(OC[C@@]56CCCN5C[C@H](F)C6)nc4c3F)c12]

### 4-(4-(3,6-dihydropyridin-1(2H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

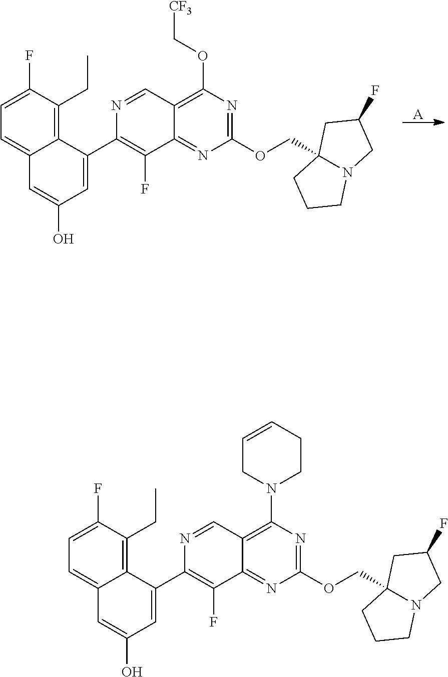

Step A. 4-(4-(3,6-dihydropyridin-1(2H)-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a mixture of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (100 mg, 1.0 equiv) and 1,2,3,6-tetrahydropyridine (60.6 mg, 2.3 equiv, HCl) in DMF (1 mL) were added DIEA (109 mg, 5.0 equiv) and 4 Å molecular sieve (10.0 mg). The reaction was stirred at 40° C. for 1.5 hours. The mixture was filtered and purified by prep-HPLC [Phenomenex luna C18 150×25 mm×10 μm; A: water (FA); B: ACN; B %: 17%-47% over 10 min] to afford the title compound (21.8 mg, 21% yield, HCOOH) as light yellow solid; ¹H NMR (400 MHz, dimethyl sulfoxide-d₆) δ=9.11 (s, 1H), 7.76 (dd, J=6.0, 9.2 Hz, 1H), 7.38-7.31 (m, 2H), 7.03 (d, J=2.4 Hz, 1H), 6.03-5.78 (m, 2H), 5.39-5.16 (m, 1H), 4.48 (br s, 2H), 4.22-4.12 (m, 1H), 4.11-3.97 (m, 3H), 3.13-2.99 (m, 3H), 2.86-2.80 (m, 1H), 2.46-2.29 (m, 3H), 2.21-2.08 (m, 2H), 2.08-1.95 (m, 2H), 1.89-1.71 (m, 3H), 0.73 (t, J=7.2 Hz, 3H); LCMS (ESI, M+1): m/z=576.2.

### Example 922

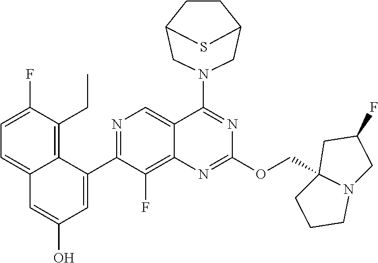 [SMILES: CCc1c(F)ccc2cc(O)cc(-c3ncc4c(N5CC6CCC(C5)S6)nc(OC[C@@]56CCCN5C[C@H](F)C6)nc4c3F)c12]

### 4-(4-(8-thia-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol

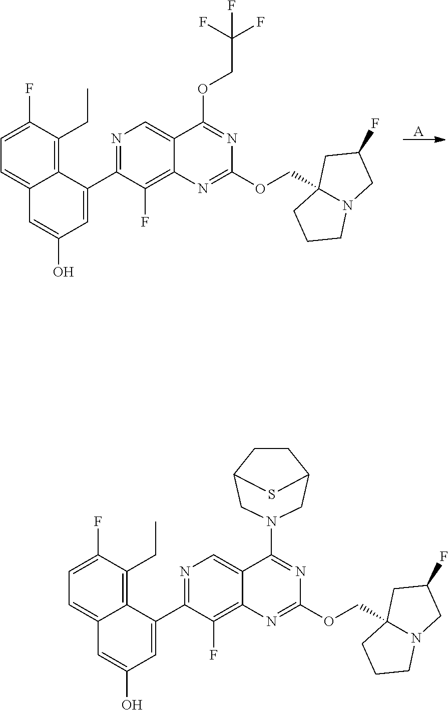

Step A. 4-(4-(8-thia-3-azabicyclo[3.2.1]octan-3-yl)-8-fluoro-2-(((2R,7aS)-2-fluorotetrahydro-1H-pyrrolizin-7a(5H)-yl)methoxy)pyrido[4,3-d]pyrimidin-7-yl)-5-ethyl-6-fluoronaphthalen-2-ol: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)naphthalen-2-ol (100 mg, 1.0 equiv) and 8-thia-3-azabicyclo[3.2.1]octane (31.7 mg, 1.5 equiv) in DMF (2 mL) was added K₄PO₃ (120 mg, 3.4 equiv). The reaction was stirred at 60° C. for 0.5 hours. The mixture was filtered and purified with prep-HPLC [Phenomenex luna C18 150×25 mm×10 μm; A: water (FA); B: ACN; B %: 17%-47% over 10 min] to afford the title compound (38.4 mg, 35% yield) as off-white solid; ¹H NMR (400 MHZ, METHANOL-d₄) δ=9.20 (br d, J=4.4 Hz, 1H), 7.68 (dd, J=5.6, 9.2 Hz, 1H), 7.35-7.21 (m, 2H), 7.06 (d, J=2.8 Hz, 1H), 5.51-5.25 (m, 2H), 4.66-4.50 (m, 1H), 4.48-4.24 (m, 3H), 3.57-3.37 (m, 4H), 3.19-3.03 (m, 3H), 2.59-2.39 (m, 2H), 2.38-2.06 (m, 9H), 2.02-1.87 (m, 1H), 0.91-0.71 (m, 3H); LCMS (ESI, M+1): m/z=622.3.

### Example 923

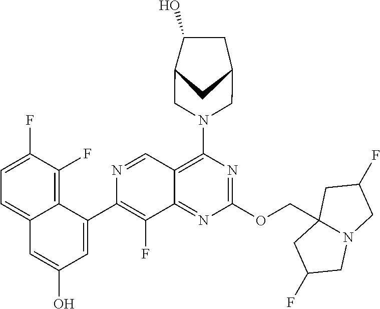 [SMILES: Oc1cc(-c2ncc3c(N4C[C@@H]5C[C@H](C4)[C@H](O)C5)nc(OCC45CC(F)CN4CC(F)C5)nc3c2F)c2c(F)c(F)ccc2c1]

### (1R,5R,6R)-3-(2-((2,6-difluorotetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol

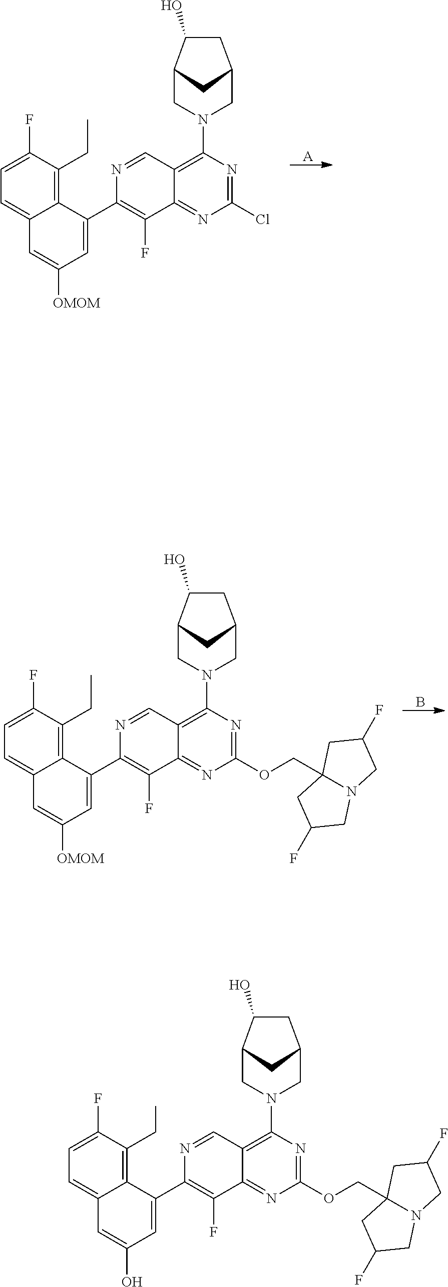

Step A. (1R,5R,6R)-3-(2-((2,6-difluorotetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (200 mg, 1.0 equiv) and (2,6-difluorotetrahydro-1H-pyrrolizin-7a(5H)-yl)methanol (98.3 mg, 1.5 equiv) in THF (1 mL) and DMF (1 mL) were added Cs₂CO₃ (361 mg, 3.0 equiv) and DABCO (41.5 mg, 1.0 equiv). The reaction was stirred at 20° C. for 12 hours. The mixture was diluted with water (15 mL) and extracted with DCM (3×15 mL). The combined organic layers were washed with brine (30 mL), dried over anhydrous sodium sulfate, concentrated, and purified with reversed phase flash chromatography [C18, 0.1% formic acid condition] to afford the title compound (100 mg, 40% yield) as yellow solid; LCMS (ESI, M+1): m/z=682.2.

Step B. (1R,5R,6R)-3-(2-((2,6-difluorotetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol: To a solution of (1R,5R,6R)-3-(2-((2,6-difluorotetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (100 mg, 1.0 equiv) in DCM (1 mL) was added TFA (1.5 mL). The reaction was stirred at 20° C. for 1 hour. The mixture was poured into saturated NaHCO₃ aqueous (10 mL) at 0° C. and extracted with DCM (3×5 mL). The combined organic layers were washed with brine (5 mL), dried over anhydrous sodium sulfate, concentrated, and purified with prep-HPLC [column: Phenomenex luna C18 150×25 mm×10 μm; A: water (FA), B: ACN, B %: 20%-40% over 10 minutes] to afford the title compound (22.1 mg, 25% yield) as white solid; ¹H NMR (400 MHZ, METHANOL-d₄) δ=9.21-9.07 (m, 1H), 7.58 (dd, J=5.6, 9.2 Hz, 1H), 7.25-7.09 (m, 2H), 6.97 (dd, J=2.4, 15.6 Hz, 1H), 5.32 (d, J=15.6 Hz, 1H), 5.19 (d, J=14.8 Hz, 1H), 4.74-4.61 (m, 1H), 4.40-4.16 (m, 3H), 3.80-3.61 (m, 1H), 3.53-3.33 (m, 2H), 3.33-3.23 (m, 2H), 3.20-3.08 (m, 2H), 2.48-2.35 (m, 2H), 2.33-2.21 (m, 3H), 2.20-1.94 (m, 4H), 1.87-1.79 (m, 1H), 1.75-1.68 (br d, J=12.0 Hz, 1H), 1.36-1.26 (m, 1H), 0.77-0.65 (m, 3H); LCMS (ESI, M+1): m/z=638.2.

### Example 924

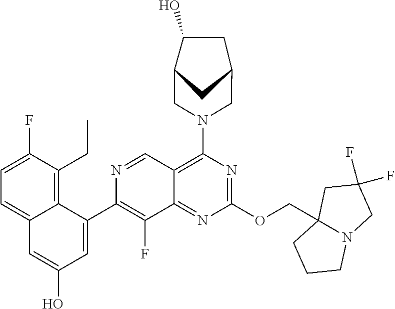 [SMILES: CCc1c(F)ccc2cc(O)cc(-c3ncc4c(N5C[C@@H]6C[C@H](C5)[C@H](O)C6)nc(OCC56CCCN5CC(F)(F)C6)nc4c3F)c12]

### (1R,5R,6R)-3-(2-((2,2-difluorotetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol

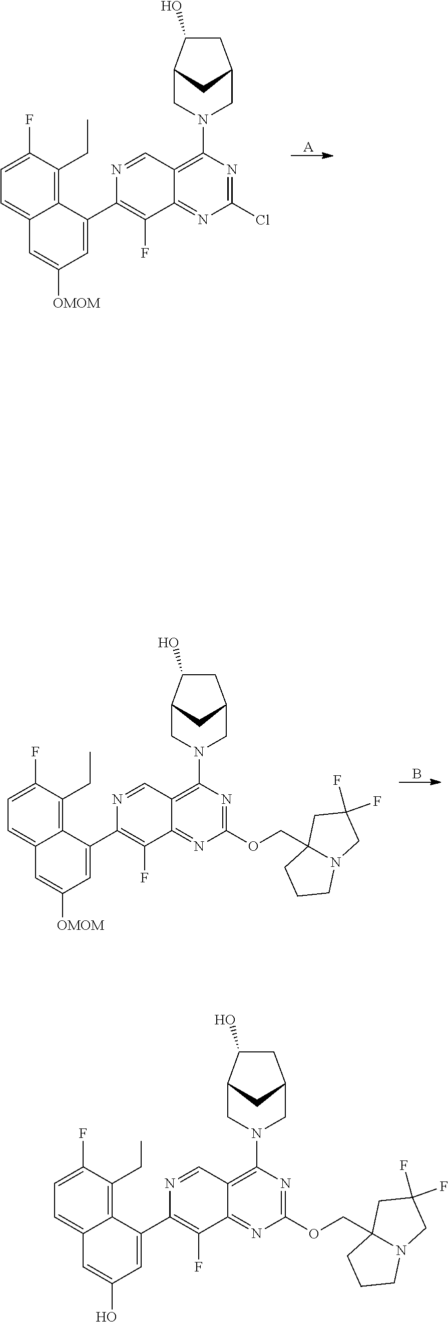

Step A. (1R,5R,6R)-3-(2-((2,2-difluorotetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (370 mg, 1.0 equiv) and (2,2-difluorotetrahydro-1H-pyrrolizin-7a(5H)-yl)methanol (182 mg, 1.5 equiv) in DMF (2 mL) and THF (2 mL) were added Cs₂CO₃ (223 mg, 1.0 equiv) and 1,4-diazabicyclo[2,2,2]octane (76.7 mg, 1.0 equiv). The reaction was stirred at 25° C. for 4 hours and 40° C. for 14 hours. The mixture was diluted with water (10 mL) and extracted with EtOAc (5×8 mL). The combined organic layers were washed with brine (10 mL), dried over anhydrous sodium sulfate, concentrated, and purified by reversed phase flash chromatography [C18, 0.1% formic acid condition] to afford the title compound (253 mg, 53% yield) as white solid; LCMS (ESI, M+1): m/z=682.3.

Step B. (1R,5R,6R)-3-(2-((2,2-difluorotetrahydro-1H-pyrrolizin-7a(5H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol: To a solution of (1R,5R,6R)-3-(2-((2,2-difluorotetrahydro-1H-pyrrolizin-7a(5H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (100 mg, 1.0 equiv) in DCM (1 mL) was added TFA (727 mg, 49.0 equiv) at 0° C. The reaction was stirred at 20° C. for 1 hour. The mixture was dropped into saturated NaHCO₃ aqueous (10 mL) at 0° C. and extracted with DCM (4×10 mL). The combined organic layers were washed with brine (10 mL), dried over anhydrous sodium sulfate, concentrated, and purified with prep-HPLC [column: Waters Xbridge 150×25 mm×5 μm; A: water (NH₄HCO₃), B: ACN, B %: 30%-60% over 8 minutes] to afford the title compound (26.9 mg, 28% yield) as white solid; ¹H NMR (400 MHZ, METHANOL-d4) δ=9.29-9.16 (m, 1H), 7.67 (dd, J=5.6, 8.8 Hz, 1H), 7.30 (d, J=2.4 Hz, 1H), 7.24 (dt, J=2.0, 9.2 Hz, 1H), 7.06 (dd, J=2.4, 15.2 Hz, 1H), 5.06-4.92 (m, 1H), 4.84-4.73 (m, 1H), 4.40-4.21 (m, 3H), 3.86-3.72 (m, 1H), 3.53-3.38 (m, 2H), 3.23-3.07 (m, 2H), 2.85 (q, J=8.0 Hz, 1H), 2.67-2.54 (m, 1H), 2.53-2.44 (m, 1H), 2.41 (br s, 1H), 2.33 (br d, J=14.0 Hz, 1H), 2.29-2.09 (m, 4H), 2.05-78 (m, 5H), 1.44-1.42 (m, 1H), 0.80 (q, J=7.2 Hz, 3H); LCMS (ESI, M+1): m/z=638.6.

### Example 925

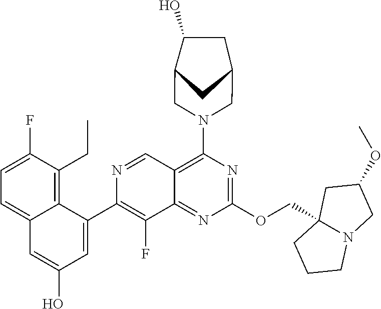 [SMILES: CCc1c(F)ccc2cc(O)cc(-c3ncc4c(N5C[C@@H]6C[C@H](C5)[C@H](O)C6)nc(OC[C@@]56CCCN5C[C@@H](OC)C6)nc4c3F)c12]

### (1R,5R,6R)-3-(7-(8-ethyl-7-fluoro-3-hydroxynaphthalen-1-yl)-8-fluoro-2-(((2S,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol

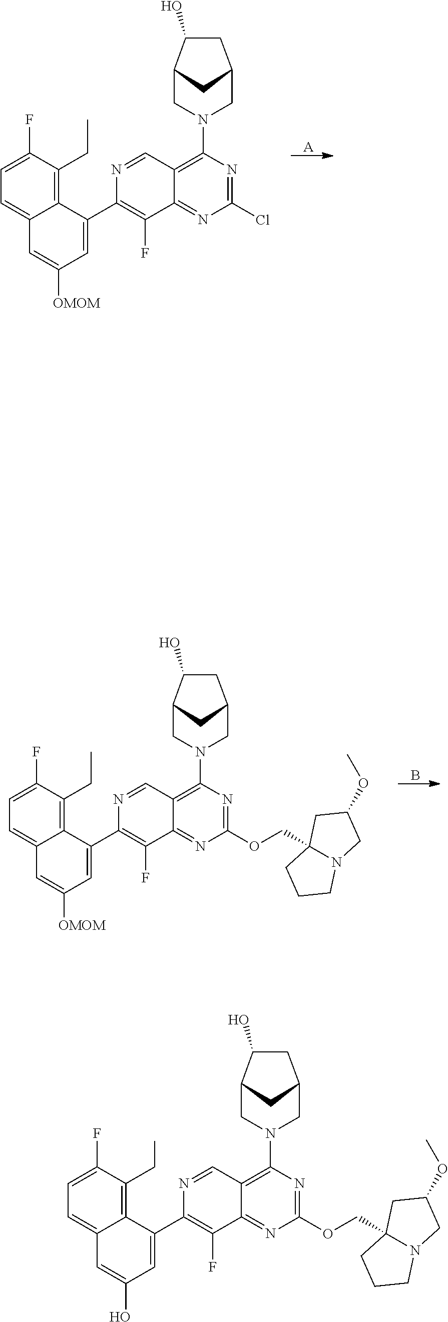

Step A. (1R,5R,6R)-3-(7-(8-ethyl-7-fluoro-3-(methoxymethoxy)naphthalen-1-yl)-8-fluoro-2-(((2S,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol: To a mixture of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (300 mg, 1.0 equiv) and ((2S,7aS)-2-methoxytetrahydro-1H-pyrrolizin-7a(5H)-yl)methanol (142 mg, 1.5 equiv) in DMF (2 mL) and THF (2 mL) were added Cs₂CO₃ (542 mg, 3.0 equiv) and DABCO (62.2 mg, 1.0 equiv). The reaction was stirred at 25° C. for 3 hours. The mixture was diluted with water (15 mL) and extracted with ethyl acetate (2×15 mL). The combined organic layers were washed with brine (10 mL), dried over anhydrous sodium sulfate, concentrated, and purified with reversed phase flash chromatography [C18, 0.1% formic acid condition] to afford the title compound (60.0 mg, 15% yield) as white solid; LCMS (ESI, M+1): m/z=676.3.

Step B. (1R,5R,6R)-3-(7-(8-ethyl-7-fluoro-3-hydroxynaphthalen-1-yl)-8-fluoro-2-(((2S,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((2S,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol (50.0 mg, 1.0 equiv) in DCM (1 mL) was added TFA (1 mL, 182 equiv) at 0° C. The reaction was stirred at 25° C. for 0.5 hours. The mixture was diluted with saturated NaHCO₃ aqueous (10 mL) and extracted with ethyl acetate (2×10 mL). The combined organic layers were washed with brine (10 mL), dried over anhydrous sodium sulfate, concentrated, and purified with prep-HPLC [column: Phenomenex luna C18 150×25 mm×10 μm; A: water (FA), B: ACN, B %: 15%-45% over 10 min] to afford the title compound (25.3 mg, 53% yield, 0.13 HCOOH) as white solid; ¹H NMR (400 MHZ, METHANOL-d₄) δ=9.31-9.22 (m, 1H), 7.69 (dd, J=6.0, 8.8 Hz, 1H), 7.32 (d, J=2.8 Hz, 1H), 7.29-7.21 (m, 1H), 7.12-7.00 (m, 1H), 5.10-4.97 (m, 1H), 4.82-4.62 (m, 3H), 4.40-4.28 (m, 1H), 4.21 (br d, J=2.8 Hz, 1H), 3.92-3.78 (m, 2H), 3.64 (br dd, J=7.2, 11.2 Hz, 1H), 3.58-3.44 (m, 1H), 3.37 (dd, J=2.0, 6.8 Hz, 4H), 3.29-3.23 (m, 1H), 2.58-2.38 (m, 4H), 2.30-2.19 (m, 5H), 2.08 (br dd, J=3.2, 12.8 Hz, 2H), 2.01-1.90 (m, 1H), 1.88-1.76 (m, 1H), 1.45-1.32 (m, 1H), 0.86-0.74 (m, 3H); LCMS (ESI, M+1): m/z=632.3

### Example 926

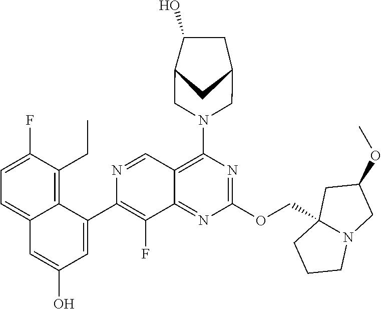 [SMILES: CCc1c(F)ccc2cc(O)cc(-c3ncc4c(N5C[C@@H]6C[C@H](C5)[C@H](O)C6)nc(OC[C@@]56CCCN5C[C@H](OC)C6)nc4c3F)c12]

### (1R,5R,6R)-3-(7-(8-ethyl-7-fluoro-3-hydroxynaphthalen-1-yl)-8-fluoro-2-(((2R,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol

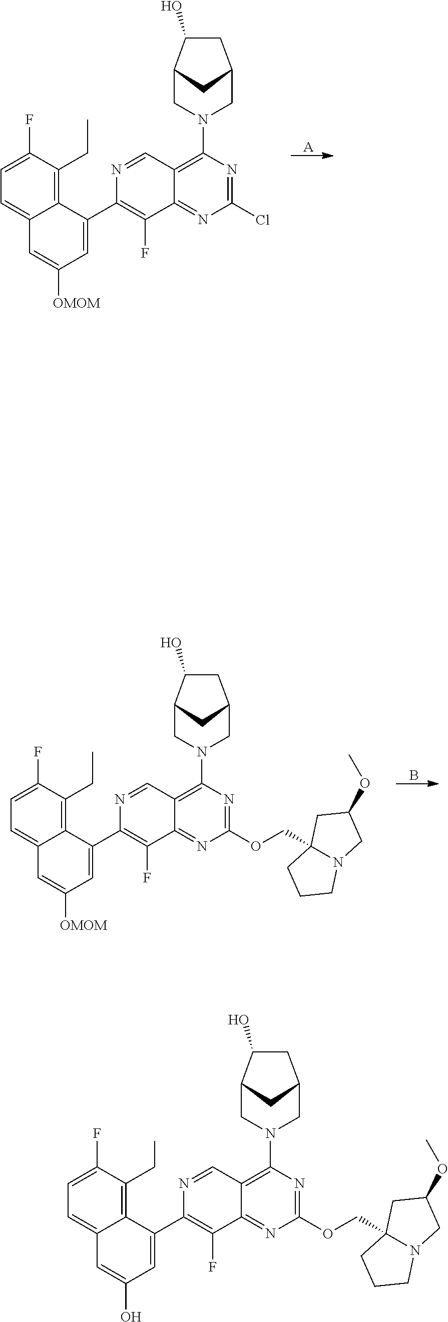

Step A. (1R,5R,6R)-3-(7-(8-ethyl-7-fluoro-3-(methoxymethoxy)naphthalen-1-yl)-8-fluoro-2-(((2R,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (207 mg, 1.0 equiv) and ((2R,7aS)-2-methoxytetrahydro-1H-pyrrolizin-7a(5H)-yl)methanol (98.3 mg, 1.5 eq) in THF (1 mL) and DMF (1 mL) were added Cs₂CO₃ (374 mg, 3.0 equiv) and DABCO (42.9 mg, 1.0 equiv). The reaction was stirred at 25° C. for 3 hours. The mixture was diluted with water (10 mL) and extracted with ethyl acetate (2×10 mL). The combined organic layers were washed with brine (10 mL), dried over anhydrous sodium sulfate, concentrated, and purified with reversed phase flash chromatography [C18, 0.1% formic acid condition] to afford the title compound (100 mg, 30% yield) as white solid; LCMS (ESI, M+1): m/z=676.4.

Step B. (1R,5R,6R)-3-(7-(8-ethyl-7-fluoro-3-hydroxynaphthalen-1-yl)-8-fluoro-2-(((2R,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((2R,7aS)-2-methoxytetrahydro-1H-pyrrolizin-7a(5H)-yl)methoxy)pyrido[4,3-d]pyrimidin-4-yl)-3-azabicyclo[3.2.1]octan-6-ol (50.0 mg, 1.0 equiv) in MeOH (0.5 mL) was added HCl·MeOH (4 M, 0.5 mL, 27 equiv) at 0° C. The reaction was stirred at 0° C. for 0.3 hours. The mixture was adjusted to pH=7 with saturated NaHCO₃ (10 mL) and extracted with DCM (2×10 mL). The combined organic layers were dried over anhydrous sodium sulfate, concentrated, and purified with [column: Phenomenex luna C18 150×25 mm×10 um; mobile phase: A: water (FA), B: ACN, B %: 16%-46% over 10 min] to afford the title compound (20.9 mg, 43% yield, 0.3HCOOH) as white solid; ¹H NMR (400 MHZ, METHANOL-d4) δ=9.33-9.15 (m, 1H), 7.68 (br dd, J=6.0, 8.4 Hz, 1H), 7.37-7.19 (m, 2H), 7.06 (dd, J=2.4, 15.2 Hz, 1H), 4.48-4.27 (m, 3H), 4.17 (br s, 1H), 3.94-3.72 (m, 1H), 3.58-3.39 (m, 3H), 3.35 (s, 3H), 3.25-3.10 (m, 4H), 2.53-2.31 (m, 3H), 2.30-2.21 (m, 2H), 2.20-2.07 (m, 5H), 1.94 (br d, J=11.6 Hz, 2H), 1.86-1.78 (m, 1H), 1.44-1.32 (m, 1H), 0.79-0.79 (m, 1H), 0.80 (br d, J=5.6 Hz, 2H); LCMS (ESI, M+1): m/z=632.3.

### Example 927

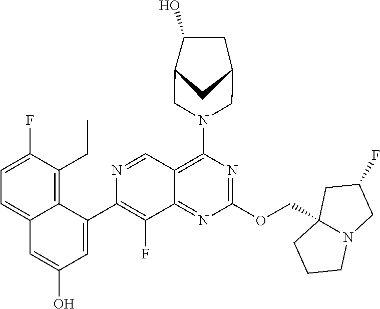 [SMILES: CCc1c(F)ccc2cc(O)cc(-c3ncc4c(N5C[C@@H]6C[C@H](C5)[C@H](O)C6)nc(OC[C@@]56CCCN5C[C@@H](F)C6)nc4c3F)c12]

### (1R,5R,6R)-3-(7-(8-ethyl-7-fluoro-3-hydroxynaphthalen-1-yl)-8-fluoro-2-(((2S,7aS)-2-fluorotetrahydro-1H-pyrrolizin-7a(5H)-yl)methoxy)pyrido[4,3-d]pyrimidin-4-yl)-3-azabicycle[3.2.1]octan-6-ol

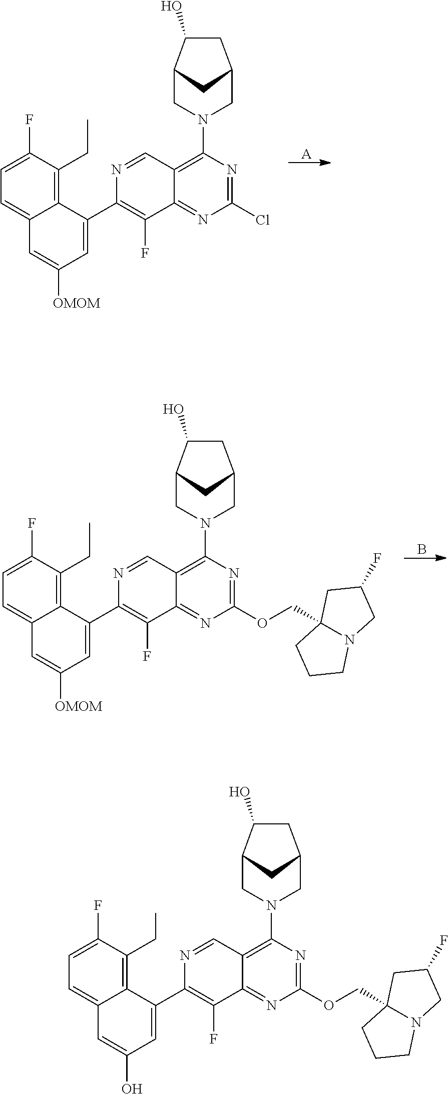

Step A. (1R,5R,6R)-3-(7-(8-ethyl-7-fluoro-3-(methoxymethoxy)naphthalen-1-yl)-8-fluoro-2-(((2S,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (300 mg, 1.0 equiv) and ((2S,7aS)-2-fluorotetrahydro-1H-pyrrolizin-7a(5H)-yl)methanol (132 mg, 1.5 equiv) in THF (1.5 mL) and DMF (1.5 mL) were added Cs₂CO₃ (542 mg, 3.0 equiv) and DABCO (62.2 mg, 1.0 equiv). The reaction was stirred at 20° C. for 12 hours. The mixture was diluted with water (5 mL) and extracted with EtOAc (3×5 mL). The combined organic layers were washed with brine (20 mL), dried over anhydrous sodium sulfate, concentrated, and purified with reversed phase flash chromatography [C18, 0.1% formic acid condition] to afford the title compound (130 mg, 34% yield) as yellow solid; LCMS (ESI, M+1): m/z=664.3.

Step B. (1R,5R,6R)-3-(7-(8-ethyl-7-fluoro-3-hydroxynaphthalen-1-yl)-8-fluoro-2-(((2S,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((2S,7aS)-2-fluorotetrahydro-1H-pyrrolizin-7a(5H)-yl)methoxy)pyrido[4,3-d]pyrimidin-4-yl)-3-azabicyclo[3.2.1]octan-6-ol (120 mg, 1.0 equiv) in MeOH (0.5 mL) was added HCl·MeOH (4 M, 1.0 mL). The reaction was stirred at 0° C. for 0.5 hours. The mixture was adjusted to pH=8 with saturated NaHCO₃ aqueous (5.0 mL) at 0° C. and extracted with EtOAc (3×5 mL). The combined organic layers were washed with brine (20 mL), dried over anhydrous sodium sulfate, concentrated, and purified with prep-HPLC [Phenomenex luna C18 150×25 mm×10 μm; A: water (FA), B: ACN; B %: 24%-45% over 7 min] and lyophilized to afford the title compound (66.6 mg, 59% yield, 0.21 HCOOH) as white solid. ¹H NMR (400 MHZ, METHANOL-d₄) δ=9.31-9.15 (m, 1H), 7.70-7.66 (m, 1H), 7.35-7.18 (m, 2H), 7.16-7.04 (m, 1H), 5.47-5.27 (m, 1H), 5.04 (br d, J=12.0 Hz, 1H), 4.81-4.72 (m, 1H), 4.52-4.40 (m, 2H), 4.32 (br dd, J=5.2, 10.4 Hz, 1H), 3.88-3.73 (m, 1H), 3.62-3.44 (m, 2H), 3.28-3.20 (m, 1H), 3.12-2.96 (m, 1H), 2.95-2.84 (m, 1H), 2.63-2.44 (m, 2H), 2.44-2.36 (m, 1H), 2.29-2.08 (m, 5H), 2.06-1.90 (m, 4H), 1.85-1.77 (m, 1H), 1.46-1.36 (m, 1H), 0.80 (dt, J=4.0, 7.2 Hz, 3H); LCMS (ESI, M+1): m/z=620.3.

### Example 928

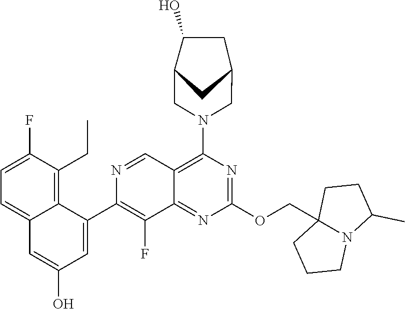 [SMILES: CCc1c(F)ccc2cc(O)cc(-c3ncc4c(N5C[C@@H]6C[C@H](C5)[C@H](O)C6)nc(OCC56CCCN5C(C)CC6)nc4c3F)c12]

### (1R,5R,6R)-3-(7-(8-ethyl-7-fluoro-3-hydroxynaphthalen-1-yl)-8-fluoro-2-((3-methyltetrahydro-1H-pyrrolizin-7a(5H)-yl)methoxy)pyrido[4,3-d]pyrimidin-4-yl)-3-azabicyclo[3.2.1]octan-6-ol

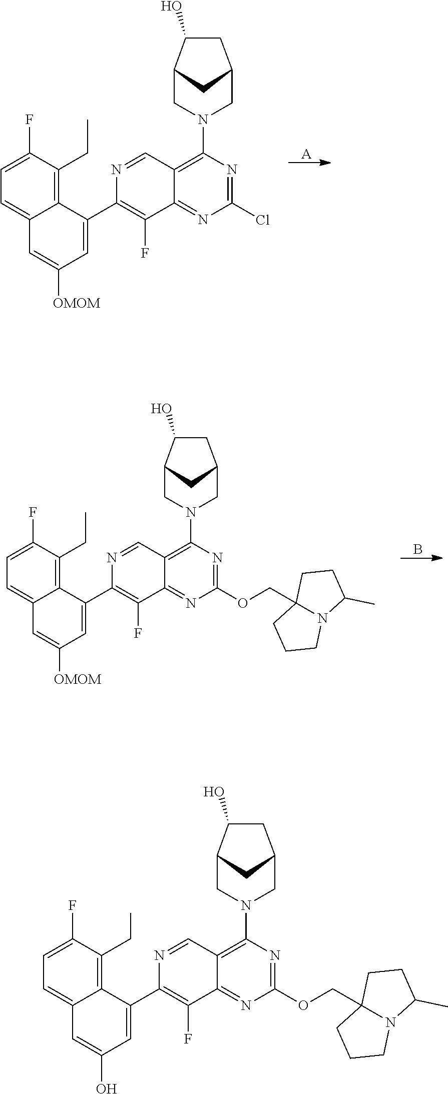

Step A. (1R,5R,6R)-3-(7-(8-ethyl-7-fluoro-3-(methoxymethoxy)naphthalen-1-yl)-8-fluoro-2-((3-methyltetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (300 mg, 1.0 equiv) and (3-methyltetrahydro-1H-pyrrolizin-7a(5H)-yl)methanol (129 mg, 1.5 equiv) in THF (1.5 mL) and DMF (1.5 mL) were added Cs₂CO₃ (542 mg, 3.0 equiv) and DABCO (62.2 mg, 1.0 equiv). The reaction was stirred at 20° C. for 12 hours. The mixture was diluted with water (5 mL) and extracted with EtOAc (3×5 mL). The combined organic layers were washed with brine (20 mL), dried over anhydrous sodium sulfate, concentrated and purified with reversed phase flash chromatography [C18, 0.1% formic acid condition] to afford the title compound (130 mg, 34% yield) as yellow solid; LCMS (ESI, M+1): m/z=660.4.

Step B. (1R,5R,6R)-3-(7-(8-ethyl-7-fluoro-3-hydroxynaphthalen-1-yl)-8-fluoro-2-((3-methyltetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-((3-methyltetrahydro-1H-pyrrolizin-7a(5H)-yl)methoxy)pyrido[4,3-d]pyrimidin-4-yl)-3-azabicyclo[3.2.1]octan-6-ol (120 mg, 1.0 equiv) in MeOH (0.5 mL) was added HCl·MeOH (4 M, 1.0 mL). The reaction was stirred at 0° C. for 0.5 hours. The mixture was adjusted to pH to 8 with saturated NaHCO₃ aqueous (5.0 mL) at 0° C. and extracted with EtOAc (3×5 mL). The combined organic layers were washed with brine (20 mL), dried over anhydrous sodium sulfate, concentrated and purified with prep-HPLC [Phenomenex luna C18 150× 25 mm×10 μm; A: water (FA), B: ACN; B %: 26%-47% over 7 min] and lyophilized to afford the title compound (61.8 mg, 54% yield, 0.51 HCOOH) as white solid. ¹H NMR (400 MHZ, METHANOL-d₄) δ=9.32-9.19 (m, 1H), 7.70-7.66 (m, 1H), 7.35-7.19 (m, 2H), 7.06 (br d, J=14.8 Hz, 1H), 5.08-4.99 (m, 1H), 4.83-4.74 (m, 1H), 4.46 (s, 2H), 4.36-4.30 (m, 1H), 3.89-3.73 (m, 1H), 3.56-3.42 (m, 1H), 3.30-3.09 (m, 3H), 2.53-2.31 (m, 3H), 2.29-2.03 (m, 7H), 2.00-1.77 (m, 5H), 1.44-1.29 (m, 4H), 0.80 (q, J=7.2 Hz, 3H); LCMS (ESI, M+1): m/z=616.3.

### Example 929

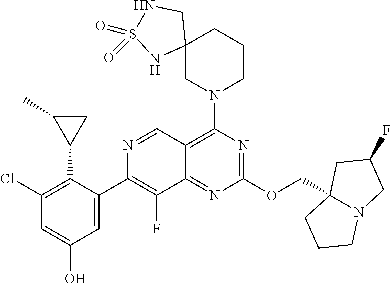 [SMILES: C[C@@H]1C[C@@H]1c1c(Cl)cc(O)cc1-c1ncc2c(N3CCCC4(CNS(=O)(=O)N4)C3)nc(OC[C@@]34CCCN3C[C@H](F)C4)nc2c1F]

### 7-(7-(3-chloro-5-hydroxy-2-(cis-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decane 2,2-dioxide

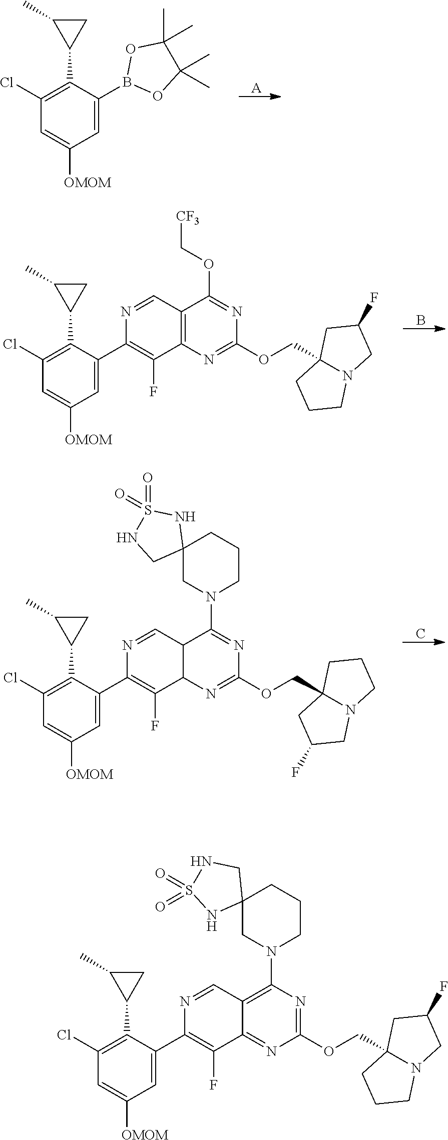

Step A. 7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine: To a solution of 2-(3-chloro-5-(methoxymethoxy)-2-((1S,2R)-2-methylcyclopropyl)phenyl)-4,4,5,5-tetramethyl-1,3,2-dioxaborolane (48.0 mg, 1.0 equiv) and 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (59.7 mg, 1.0 equiv) in methoxycyclopentane (1 mL) and water (0.25 mL) were added Cs₂CO₃ (44.3 mg, 1.0 equiv) and CataCXium A Pd G3 (19.8 mg, 0.2 equiv). The reaction was degassed and purged with N₂ for 3 times. The reaction was stirred at 90° C. for 10 hours under N₂ atmosphere. The mixture was diluted with water (30 mL) and extracted with ethyl acetate (2×30 mL). The combined organic layers were washed with brine (40 mL), dried over anhydrous sodium sulfate, concentrated and purified with prep-TLC [SiO₂, petroleum ether/ethyl acetate=0/1] to afford the title compound (16.0 mg, 17% yield) as yellow solid; LCMS (ESI, M+1): m/z=629.3.

Step B. 7-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decane 2,2-dioxide: To a solution of 7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (13.0 mg, 1.0 equiv) and 2-thia-1,3,7-triazaspiro[4.5]decane 2,2-dioxide (5.93 mg, 1.5 equiv) in DMF (1 mL) were added DIEA (8.01 mg, 3.0 equiv) and 4 Å molecular sieve (5 mg). The reaction was stirred at 45° C. for 12 hours. The mixture was diluted with water (10 mL) and extracted with ethyl acetate (2×10 mL). The combined organic layers were washed with brine (10 mL), dried over anhydrous sodium sulfate, concentrated and purified with prep-TLC [SiO₂, DCM/MeOH=10/1] to afford the title compound (12.0 mg, 77% yield) as yellow solid; LCMS (ESI, M+1): m/z=720.2.

Step C. 7-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decane 2,2-dioxide: To a solution of 7-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-2-thia-1,3,7-triazaspiro[4.5]decane 2,2-dioxide (10.0 mg, 1.0 equiv) in MeOH (2 mL) was added HCl·MeOH (4M, 2 mL). The reaction was stirred at 0° C. for 2 hours. The mixture was concentrated, dissolved in MeOH (2 mL), adjusted to pH=7 with solid NaHCO₃ and filtered. The filtration was concentrated and purified with prep-HPLC [column: Waters Xbridge 150×25 mm×5 um; mobile phase: water (NH₄HCO₃)-ACN; gradient: 35%-65% B over 9 min] to afford the title compound (6.58 mg, 70% yield) as off-white solid; ¹H NMR (400 MHZ, METHANOL-d₄) δ=9.09 (d, J=10.4 Hz, 1H), 7.02 (d, J=2.0 Hz, 1H), 6.84 (br d, J=4.0 Hz, 1H), 5.47-5.20 (m, 1H), 4.64-4.48 (m, 1H), 4.46-4.24 (m, 3H), 3.93-3.64 (m, 2H), 3.48-3.39 (m, 1H), 3.38-3.34 (m, 1H), 3.31-3.13 (m, 4H), 3.09-2.98 (m, 1H), 2.43-1.84 (m, 12H), 1.13 (br s, 1H), 0.67 (br s, 4H); LCMS (ESI, M+1): m/z=676.3.

### Example 930

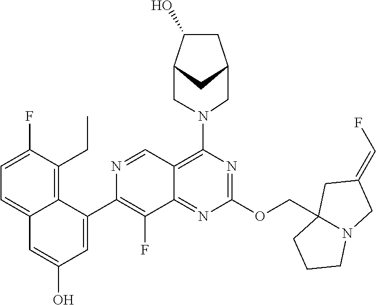 [SMILES: CCc1c(F)ccc2cc(O)cc(-c3ncc4c(N5C[C@@H]6C[C@H](C5)[C@H](O)C6)nc(OCC56CCCN5C/C(=C/F)C6)nc4c3F)c12]

### (1R,5R,6R)-3-(7-(8-ethyl-7-fluoro-3-hydroxynaphthalen-1-yl)-8-fluoro-2-(((E)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol

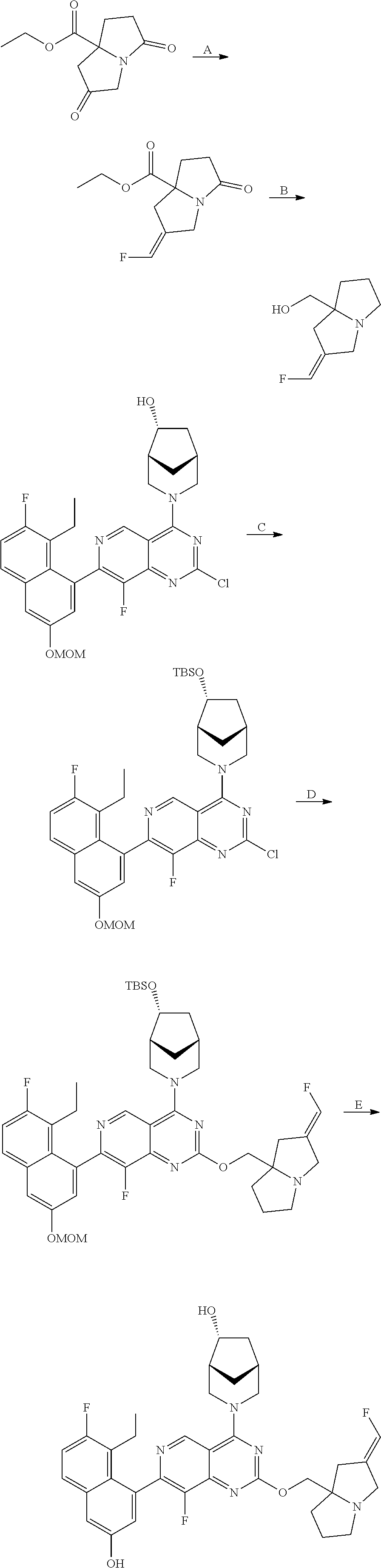

Step A. ethyl (E)-2-(fluoromethylene)-5-oxotetrahydro-1H-pyrrolizine-7a(5H)-carboxylate: To a solution of 2-((fluoromethyl) sulfonyl)pyridine (9.12 g, 1.1 equiv) in THF (100 mL) was added KHMDS (1 M, 60.1 mL, 1.2 equiv) dropwise at −65° C. The reaction was stirred at −65° C. for 1 hour. Ethyl 2,5-dioxotetrahydro-1H-pyrrolizine-7a(5H)-carboxylate (10 g, 1.0 equiv) in THF (50 mL) was added dropwise at −65° C. The reaction was stirred at −65° C. for 2 hours and 20° C. for 2 hours. The mixture was quenched with 0.1N HCl (30 mL) at 0° C. The pH of the mixture was adjusted to 3 with 2N HCl. The mixture was extracted with EtOAc (4×100 mL). The combined organic layers were washed with brine (50 mL), dried over anhydrous Na₂SO₄, concentrated and purified with column chromatography [SiO2, PE/EtOAc=2/1 to 1/1] followed by purified with prep-HPLC [column: UniSil 10-120 C18 70×250 mm; A: water (FA), B: ACN; B %: 15%-45% over 20 min] to afford the title compound (860 mg, 8.6% yield) as yellow oil; ¹H NMR (400 MHZ, CHLOROFORM-d) δ=6.75-6.48 (m, 1H), 4.35 (br d, J=14.8 Hz, 1H), 4.23 (q, J=7.2 Hz, 2H), 3.73 (br d, J=14.8 Hz, 1H), 3.32 (br d, J=16.4 Hz, 1H), 2.87-2.74 (m, 1H), 2.73-2.62 (m, 1H), 2.53-2.39 (m, 2H), 2.15 (td, J=10.0, 13.2 Hz, 1H), 1.36-1.26 (m, 3H); LCMS (ESI, M+1): m/z=228.0.

Step B. (E)-(2-(fluoromethylene)tetrahydro-1H-pyrrolizin-7a(5H)-yl)methanol: To a solution of ethyl (E)-2-(fluoromethylene)-5-oxotetrahydro-1H-pyrrolizine-7a(5H)-carboxylate (300 mg, 1.0 equiv) in THF (6.00 mL) was added DIBAL-H (1 M, 10 equiv) at 0° C. The reaction was stirred at 0° C. for 2 hours. The mixture was quenched with water (0.6 mL), then 15% NaOH (0.6 mL) and water (1.8 mL). The mixture was dried over anhydrous sodium sulfate, concentrated to afford the title compound (130 mg, crude) as yellow oil; ¹H NMR (400 MHZ, CHLOROFORM-d) δ=6.67-6.40 (m, 1H), 3.61-3.51 (m, 1H), 3.49-3.32 (m, 1H), 3.31-3.20 (m, 2H), 3.13-3.00 (m, 1H), 2.61 (td, J=7.6, 10.0 Hz, 1H), 2.56-2.35 (m, 2H), 2.03-1.65 (m, 4H); LCMS (ESI, M+1): m/z=172.1.

Step C. 4-((1R,5R,6R)-6-((tert-butyldimethylsilyl)oxy)-3-azabicyclo[3.2.1]octan-3-yl)-2-chloro-7-(8-ethyl-7-fluoro-3-(methoxymethoxy)naphthalen-1-yl)-8-fluoropyrido[4,3-d]pyrimidine: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (690 mg, 1.0 equiv), TBSCl (384 mg, 2.0 equiv) and imidazole (260 mg, 3.0 equiv) in DMF (5.00 mL) was added DMAP (77.9 mg, 0.5 equiv). The reaction was stirred at 40° C. for 5 hours. The mixture was diluted with water (20 mL) and extracted with ethyl acetate (2×10 mL). The combined organic layers were dried over anhydrous sodium sulfate, concentrated and purified with reversed phase flash chromatography [C18, 0.1% formic acid condition] to afford the title compound (510 mg, 49% yield) as yellow solid; LCMS (ESI, M+1): m/z=655.3.

Step D. 4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-8-fluoro-2-(((E)-2-(fluoromethylene)tetrahydro-1H-pyrrolizin-7a(5H)-yl)methoxy)pyrido[4,3-d]pyrimidine: To a solution of (E)-(2-(fluoromethylene)tetrahydro-1H-pyrrolizin-7a(5H)-yl)methanol (39.2 mg, 1.5 equiv) in THF (2.00 mL) was added NaH (18.3 mg, 60% purity, 3.0 equiv) at 0° C. The reaction was stirred at 0° C. for 10 minutes. 4-((1R,5R,6R)-6-((tert-butyldimethylsilyl)oxy)-3-azabicyclo[3.2.1]octan-3-yl)-2-chloro-7-(8-ethyl-7-fluoro-3-(methoxymethoxy)naphthalen-1-yl)-8-fluoropyrido[4,3-d]pyrimidine (100 mg, 1.0 equiv) was added. The reaction was stirred at 0-20° C. for 2 hours. The mixture quenched with water (20 mL) and extracted with ethyl acetate (2×10 mL). The combined organic layers were dried over anhydrous sodium sulfate, concentrated and purified with reversed phase flash chromatography [C18, 0.1% formic acid condition] to afford the title compound (65.0 mg, 52% yield) as yellow solid; LCMS (ESI, 1/2M+1,M+1): m/z=395.9, 790.4.

Step E. (1R,5R,6R)-3-(7-(8-ethyl-7-fluoro-3-hydroxynaphthalen-1-yl)-8-fluoro-2-(((E)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol: To a solution of 4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-8-fluoro-2-(((E)-2-(fluoromethylene)tetrahydro-1H-pyrrolizin-7a(5H)-yl)methoxy)pyrido[4,3-d]pyrimidine (55.0 mg, 1.0 equiv) in MeOH (2.00 mL) was added HCl·MeOH (4 M, 2.00 mL) at 0° C. The reaction was stirred at 20° C. for 1.5 hours. The mixture was concentrated, basified by saturated NaHCO₃ solution (20 mL) and extracted with ethyl acetate (2×10 mL) The combined organic layers were washed with brine (10 mL), dried over anhydrous sodium sulfate, concentrated, and purified with prep-HPLC [column: C18 150×30 mm; A: water (FA),B: ACN; B %: 15%-45% 12 min] to afford the title compound (12.8 mg, 26% yield) as white solid; ¹H NMR (400 MHZ, DMSO-d6) δ=10.07-9.79 (m, 1H), 9.38-9.22 (m, 1H), 8.14 (s, 1H), 7.76 (dd, J=6.4, 8.4 Hz, 1H), 7.41-7.27 (m, 2H), 7.03 (dd, J=2.0, 14.4 Hz, 1H), 6.96-6.69 (m, 1H), 4.93-4.50 (m, 3H), 4.24-3.95 (m, 3H), 3.80-3.67 (m, 1H), 3.56 (br d, J=13.6 Hz, 1H), 3.24 (br s, 1H), 3.05-2.94 (m, 1H), 2.76-2.62 (m, 1H), 2.59-2.52 (m, 1H), 2.47-2.25 (m, 4H), 2.18-2.03 (m, 3H), 2.02-1.82 (m, 2H), 1.81-1.59 (m, 4H), 1.25 (br d, J=12.8 Hz, 1H), 0.78-0.64 (m, 3H); LCMS (ESI, 1/2M+1, M+1): m/z=316.8, 632.3.

### Example 931

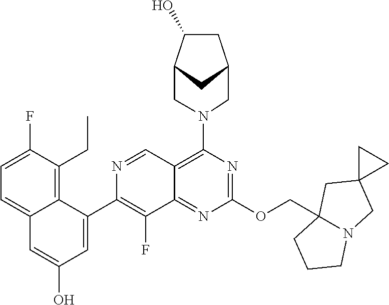 [SMILES: CCc1c(F)ccc2cc(O)cc(-c3ncc4c(N5C[C@@H]6C[C@H](C5)[C@H](O)C6)nc(OCC56CCCN5CC5(CC5)C6)nc4c3F)c12]

### (1R,5R,6R)-3-(2-((dihydro-1′H,3′H-spiro[cyclopropane-1,2′-pyrrolizin]-7a′ (5′H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol

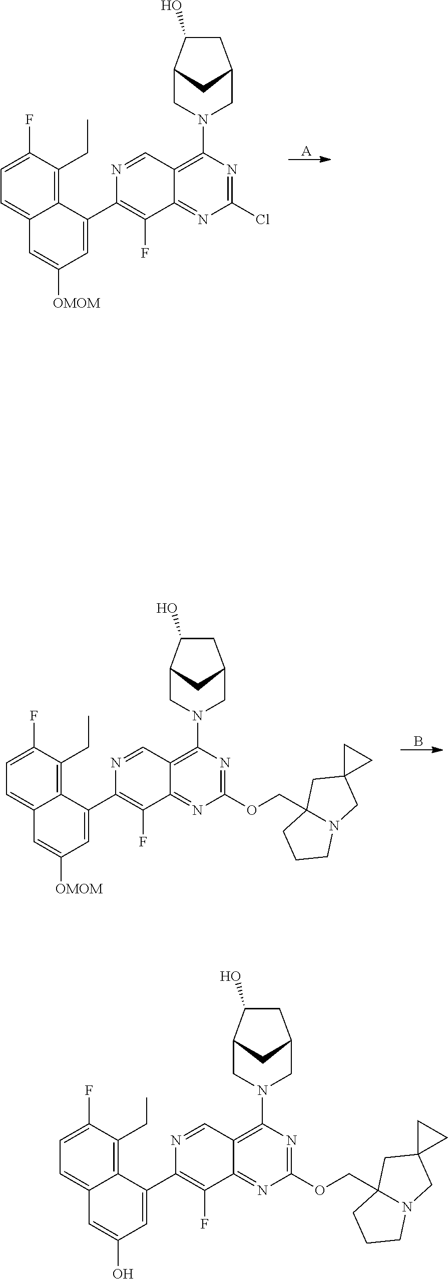

Step A. (1R,5R,6R)-3-(2-((dihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (300 mg, 1.0 equiv) and (dihydro-1′H,3′H-spiro[cyclopropane-1,2′-pyrrolizin]-7a′ (5′H)-yl)methanol (139 mg, 1.5 equiv) in THF (3 mL) and DMF (3 ml) were added Cs₂CO₃ (542 mg, 3.0 equiv) and DABCO (62.0 mg, 1.0 equiv). The reaction was stirred at 25° C. for 3 hours. The mixture was diluted with water (15 mL) and extracted with EtOAc (2×15 mL). The combined organic layers were dried over anhydrous sodium sulfate, concentrated and purified with reversed phase flash chromatography [0.1% formic acid condition] to afford the title compound (80.0 mg, 12% yield) as yellow solid; LCMS (ESI, M+1): m/z=672.3.

Step B. (1R,5R,6R)-3-(2-((dihydro-1′H,3′H-spiro[cyclopropane-1,2′-pyrrolizin]-7a′ (5′H)-yl)methoxy)-7-(8-ethyl-7-fluoro-3-hydroxynaphthalen-1-yl)-8-fluoropyrido[4,3-d]pyrimidin-4-yl)-3-azabicyclo[3.2.1]octan-6-ol: To a solution of (1R,5R,6R)-3-(2-((dihydro-1′H,3′H-spiro[cyclopropane-1,2′-pyrrolizin]-7a′ (5′H)-yl)methoxy)-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (70.0 mg, 1.0 equiv) in DCM (1 mL) was added TFA (3.07 g, 258 equiv) at 0° C. The reaction was stirred at 25° C. for 0.5 hours. The mixture was concentrated under vacuum. The residue was diluted with water (1 mL). The mixture was adjusted to pH=7 with NaHCO₃ aqueous (10 mL) and extracted with EtOAc (2×10 mL). The combined organic layers were washed with brine (10 mL), dried over anhydrous sodium sulfate, concentrated and purified with HPLC [column: Phenomenex luna C18 150×25 mm×10 μm; A: water (FA), B: ACN, B %: 17%-47% over 10 min] to afford the title compound (5.53 mg, 8% yield, 0.70 HCOOH) as off-white solid; ¹H NMR (400 MHZ, METHANOL-d4) δ=9.26 (d, J=19.6 Hz, 1H), 7.68 (dd, J=6.0, 9.2 Hz, 1H), 7.31 (d, J=2.8 Hz, 1H), 7.28-7.22 (m, 1H), 7.06 (dd, J=2.4, 16.8 Hz, 1H), 4.60 (br d, J=12.0 Hz, 4H), 4.38-4.30 (m, 1H), 3.89-3.77 (m, 1H), 3.59-3.38 (m, 3H), 3.28-3.22 (m, 1H), 3.15-3.09 (m, 1H), 2.51-2.39 (m, 2H), 2.30-2.02 (m, 9H), 1.98-1.92 (m, 1H), 1.86-1.79 (m, 1H), 1.43-1.35 (m, 1H), 0.80 (q, J=7.2 Hz, 5H), 0.74-0.67 (m, 2H); LCMS (ESI, M+1): m/z=628.3.

### Example 932

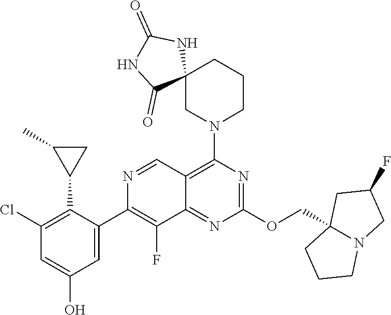 [SMILES: C[C@@H]1C[C@@H]1c1c(Cl)cc(O)cc1-c1ncc2c(N3CCC[C@]4(C3)NC(=O)NC4=O)nc(OC[C@@]34CCCN3C[C@H](F)C4)nc2c1F]

### (R)-7-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione

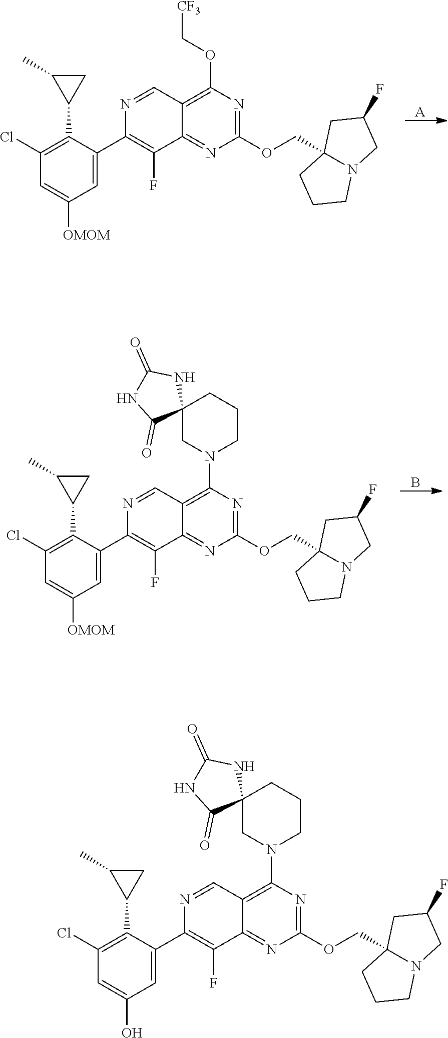

Step A. (R)-7-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione: To a solution of 7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) and (R)-1,3,7-triazaspiro[4.5]decane-2,4-dione (80.7 mg, 3.0 equiv) in DMF (3 mL) were added 4 Å molecular sieves (50.0 mg) and DIEA (103 mg, 5 equiv). The reaction was stirred at 40° C. for 12 hours. The mixture was diluted with water (20 mL) and extracted with ethyl acetate (2×30 mL). The combined organic layers were washed with brine (20 mL), dried over anhydrous sodium sulfate, concentrated and purified with prep-HPLC [0.1% FA condition] to afford the title compound (50.0 mg, 44% yield) as white solid; LCMS (ESI, M+1): m/z=698.1.

Step B. (R)-7-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione: To a solution of (R)-7-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-1,3,7-triazaspiro[4.5]decane-2,4-dione (50.0 mg, 1.0 equiv) in MeCN (2 mL) was added HCl·dioxane (4M, 2 mL). The reaction was stirred at 0° C. for 1 hour. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 um; mobile phase: water (NH₄HCO₃)-ACN; gradient: 34%-64% B over 9 min] to afford the title compound (29.0 mg, 62% yield) as white solid; ¹H NMR (400 MHZ, DMSO-d₆) δ=10.87 (s, 1H), 9.98 (s, 1H), 9.07 (d, J=6.4 Hz, 1H), 8.70 (s, 1H), 6.99 (d, J=2.4 Hz, 1H), 6.82 (s, 1H), 5.51-5.02 (m, 1H), 4.65-4.26 (m, 2H), 4.14-4.02 (m, 2H), 3.57-3.44 (m, 2H), 3.15-2.98 (m, 3H), 2.88-2.75 (m, 1H), 2.13 (s, 12H), 1.12-0.97 (m, 1H), 0.65-0.45 (m, 4H); LCMS (ESI, M+1): m/z=654.2.

### Example 933

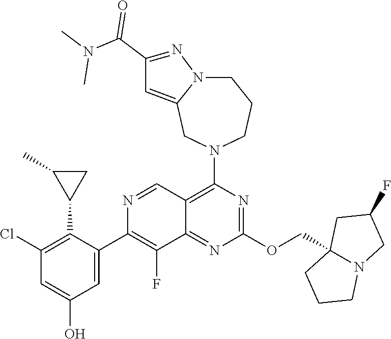 [SMILES: C[C@@H]1C[C@@H]1c1c(Cl)cc(O)cc1-c1ncc2c(N3CCCn4nc(C(=O)N(C)C)cc4C3)nc(OC[C@@]34CCCN3C[C@H](F)C4)nc2c1F]

### 5-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

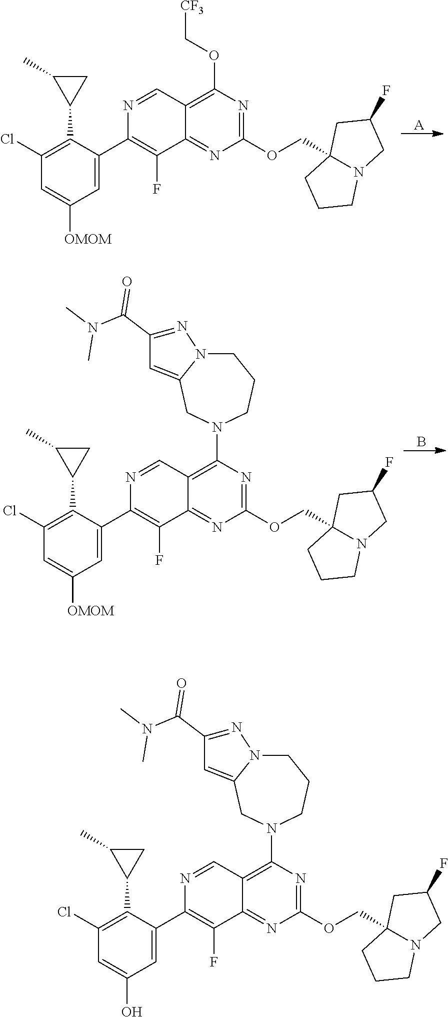

Step A. 5-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (100 mg, 1.0 equiv) and N,N-dimethyl-5,6,7,8-tetrahydro-4H-pyrazolo[1,5-a][1,4]diazepine-2-carboxamide (66.2 mg, 2.0 equiv) in DMF (3 mL) were added 4 Å molecular sieve (50.0 mg) and K₃PO₄ (103 mg, 5.0 equiv).

The reaction was stirred at 60° C. for 12 hours. The mixture was diluted with water (15 mL) and extracted with ethyl acetate (3× 15 mL). The combined organic layers were washed with brine (20 mL), dried over anhydrous sodium sulfate, concentrated and purified with prep-HPLC [0.1% FA condition] to afford the title compound (35.0 mg, 30% yield) as white solid; LCMS (ESI, M+1): m/z=737.1.

Step B. 5-(7-(3-chloro-5-hydroxy-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 5-(7-(3-chloro-5-(methoxymethoxy)-2-((1S,2R)-2-methylcyclopropyl)phenyl)-8-fluoro-2-(((2R,7aS)-2-fluorotetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (50.0 mg, 1.0 equiv) in MeCN (1.5 mL) was added HCl·dioxane (4M, 1.5 mL). The reaction was stirred at 0° C. for 1 hour. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 um; mobile phase: water (NH₄HCO₃)-ACN; gradient: 38%-68% B over 9 min] to afford the title compound (6.38 mg, 13% yield) as white solid; ¹H NMR (400 MHZ, DMSO-d₆)=9.15 (s, 1H), 7.00 (d, J=2.4 Hz, 1H), 6.82 (d, J=2.0 Hz, 1H), 6.76 (s, 1H), 5.43-5.20 (m, 3H), 4.55 (dd, J=2.8, 6.8 Hz, 2H), 4.42 (s, 2H), 4.31-4.20 (m, 2H), 3.33 (s, 3H), 3.29-3.15 (m, 3H), 3.08 (s, 3H), 3.04-2.98 (m, 1H), 2.51-1.82 (m, 10H), 1.17-1.01 (m, 1H), 0.70-0.55 (m, 4H); LCMS (ESI, M+1): m/z=693.1.

### Example 934

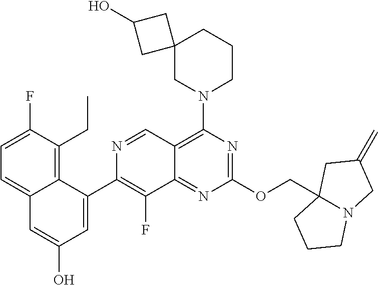 [SMILES: C=C1CN2CCCC2(COc2nc(N3CCCC4(CC(O)C4)C3)c3cnc(-c4cc(O)cc5ccc(F)c(CC)c45)c(F)c3n2)C1]

### 6-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-6-azaspiro[3.5]nonan-2-ol

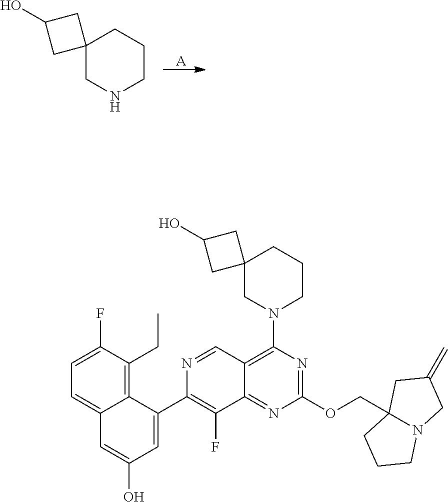

Step A. 6-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-6-azaspiro[3.5]nonan-2-ol: To a solution of 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol (50.0 mg, 1.0 equiv) and 6-azaspiro[3.5]nonan-2-ol (18.1 mg, 1.5 equiv) in DMF (1 mL) were added 4 Å molecular sieves (50 mg) and DIEA (33.1 mg, 3.0 equiv). The reaction was stirred at 40° C. for 10 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 40%-70% B over 9 minutes] to afford the title compound (9.48 mg, 16% yield) as white solid; ¹H NMR (400 MHZ, METHANOL-d₄) δ=9.05 (d, J=6.4 Hz, 1H), 7.67 (dd, J=6.0, 9.2 Hz, 1H), 7.33-7.20 (m, 2H), 7.07 (t, J=2.4 Hz, 1H), 4.99 (br s, 2H), 4.37-4.19 (m, 3H), 4.14-3.87 (m, 4H), 3.73 (br d, J=14.0 Hz, 1H), 3.15 (td, J=4.8, 10.0 Hz, 1H), 2.84-2.66 (m, 2H), 2.55-2.43 (m, 2H), 2.33-2.11 (m, 4H), 2.04-1.66 (m, 10H), 0.86-0.75 (m, 3H); LCMS (ESI, M+1): m/z=628.3.

### Example 935

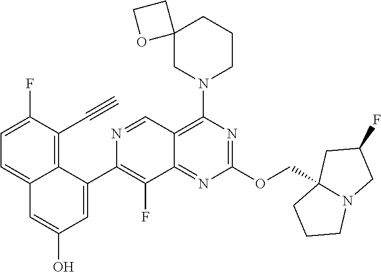 [SMILES: C#Cc1c(F)ccc2cc(O)cc(-c3ncc4c(N5CCCC6(CCO6)C5)nc(OC[C@@]56CCCN5C[C@H](F)C6)nc4c3F)c12]

### 5-ethynyl-6-fluoro-4-(8-fluoro-2-(((2R,7aS)-2-fluorotetrahydro-1H-pyrrolizin-7a(5H)-yl)methoxy)-4-(1-oxa-6-azaspiro[3.5]nonan-6-yl)pyrido[4,3-d]pyrimidin-7-yl)naphthalen-2-ol

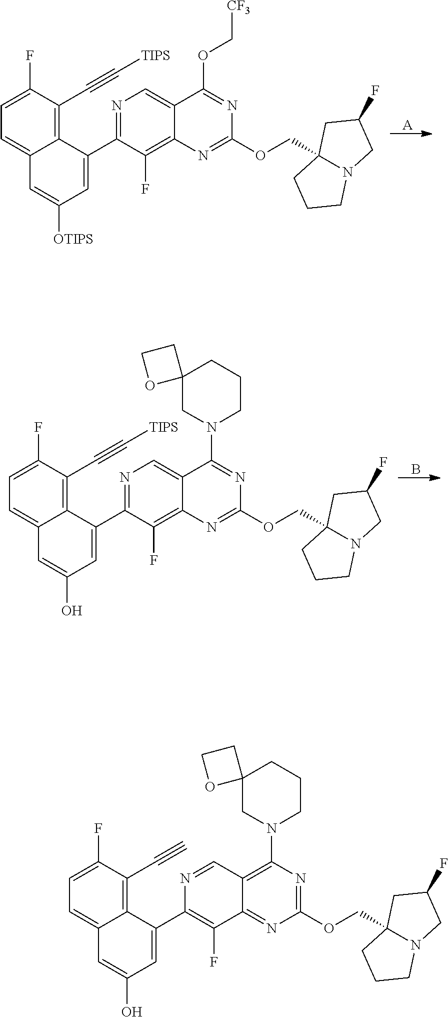

Step A. 6-fluoro-4-(8-fluoro-2-(((2R,7aS)-2-fluorotetrahydro-1H-pyrrolizin-7a(5H)-yl)methoxy)-4-(1-oxa-6-azaspiro[3.5]nonan-6-yl)pyrido[4,3-d]pyrimidin-7-yl)-5-((triisopropylsilyl)ethynyl)naphthalen-2-ol: To a mixture of 8-fluoro-7-(7-fluoro-8-((triisopropylsilyl)ethynyl)-3-((triisopropylsilyl)oxy)naphthalen-1-yl)-2-(((2R,7aS)-2-fluorotetrahydro-1H-pyrrolizin-7a(5H)-yl)methoxy)-4-(2,2,2-trifluoroethoxy)pyrido[4,3-d]pyrimidine (60.0 mg, 1.0 equiv), 1-oxa-8-azaspiro[3.5]nonane (68.8 mg, 3.0 equiv, oxalic) and 4 Å molecular sieve (10.0 mg, 1.0 equiv) in DMF (1 mL) was added DIEA (25.8 mg, 3.0 equiv). The reaction was stirred at 60° C. for 12 hours. The mixture was diluted with H₂O (2 mL) and extracted with EtOAc (3×2 mL). The combined organic layers were washed with brine (2 mL), dried over anhydrous sodium sulfate, filtered and purified with reversed phase flash chromatography [C18, 0.1% formic acid condition] to afford the title compound (50 mg, 78% yield) as yellow solid; LCMS (ESI, M+1): m/z=772.5.

Step B. 5-ethynyl-6-fluoro-4-(8-fluoro-2-(((2R,7aS)-2-fluorotetrahydro-1H-pyrrolizin-7a(5H)-yl)methoxy)-4-(1-oxa-6-azaspiro[3.5]nonan-6-yl)pyrido[4,3-d]pyrimidin-7-yl)naphthalen-2-ol: To a solution of 6-fluoro-4-(8-fluoro-2-(((2R,7aS)-2-fluorotetrahydro-1H-pyrrolizin-7a(5H)-yl)methoxy)-4-(1-oxa-6-azaspiro[3.5]nonan-6-yl)pyrido[4,3-d]pyrimidin-7-yl)-5-((triisopropylsilyl)ethynyl)naphthalen-2-ol (40.0 mg, 1.0 equiv) in DMF (1 mL) was added CsF (65.5 mg, 10 equiv). The reaction was stirred at 40° C. for 12 hours. The mixture was diluted with H₂O (2 mL) and extracted with EtOAc (3×2 mL). The combined organic layers were washed with brine (2 mL), dried over anhydrous sodium sulfate, filtered and purified by prep-HPLC (Waters Xbridge C18 150×25 mm×5 μm; A: water (10 mM NH₄HCO₃); B: ACN, B %: 35%-65% over 9 min) to afford the title compound (17.0 mg, 63% yield) as yellow solid. ¹H NMR (400 MHz, CHLOROFORM-d) δ=9.24-8.95 (m, 1H), 7.61 (br t, J=6.4 Hz, 1H), 7.25-7.07 (m, 3H), 5.41-5.17 (m, 1H), 4.65-4.13 (m, 6H), 3.62 (br t, J=14.4 Hz, 1H), 3.50-3.14 (m, 4H), 3.05-2.94 (m, 1H), 2.84 (d, J=16.0 Hz, 1H), 2.47-2.37 (m, 2H), 2.32-2.20 (m, 3H), 2.12 (br d, J=8.0 Hz, 2H), 1.94 (dt, J=6.0, 11.6 Hz, 3H), 1.82-1.70 (m, 2H); LCMS (ESI, M+1): m/z=616.4.

### Example 936

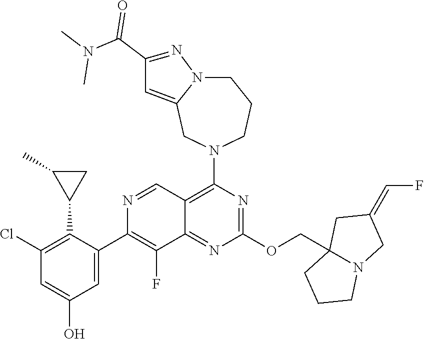 [SMILES: C[C@@H]1C[C@@H]1c1c(Cl)cc(O)cc1-c1ncc2c(N3CCCn4nc(C(=O)N(C)C)cc4C3)nc(OCC34CCCN3C/C(=C\F)C4)nc2c1F]

### 5-(7-(3-chloro-5-hydroxy-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide

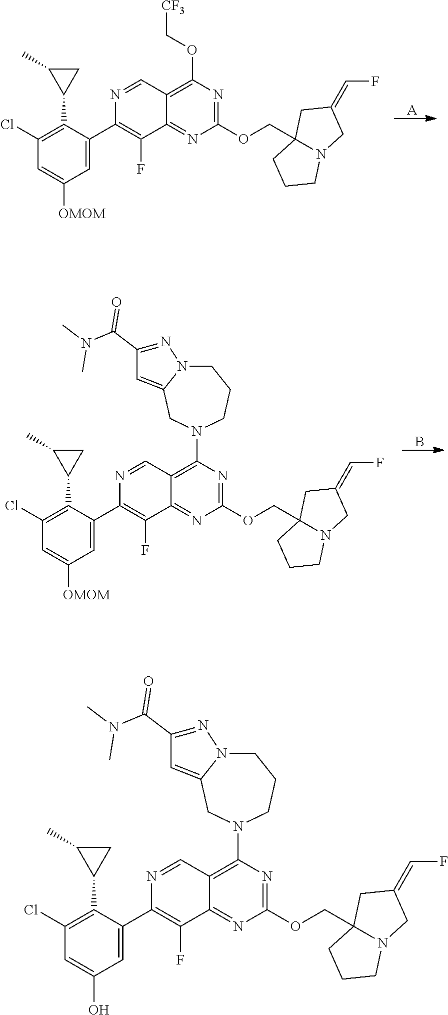

Step A. 5-(7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)-4-(2,2,2-trifluoroethoxy)pyrido[4,3-d]pyrimidine (90.0 mg, 1.0 equiv) and N,N-dimethyl-5,6,7,8-tetrahydro-4H-pyrazolo[1,5-a][1,4]diazepine-2-carboxamide (87.7 mg, 3.0 equiv) in DMF (2 mL) were added K₃PO₄ (447 mg, 15 equiv) and 4 Å molecular sieve (50.0 mg). The reaction was stirred at 80° C. for 12 hours. The mixture was diluted with water (10 mL) and extracted with ethyl acetate (3×10 mL). The combined organic layers were washed with brine (20 mL), dried over anhydrous sodium sulfate, concentrated and purified with prep-TLC [SiO₂, dichloromethane/methanol=10/1] to afford the title compound (40.0 mg, 35% yield) as yellow solid; LCMS (ESI, M+1): m/z=749.3.

Step B. 5-(7-(3-chloro-5-hydroxy-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide: To a solution of 5-(7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-N,N-dimethyl-5,6,7,8-tetrahydro-4H-pyrazolo[1,5-a][1,4]diazepine-2-carboxamide (33.0 mg, 1.0 equiv) in MeCN (1 mL) was added HCl·dioxane (4M, 1 mL) at 0° C. The reaction was stirred at 0° C. for 1 hour. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 um; mobile phase: water (NH₄HCO₃)-ACN; gradient: 40%-70% B over 9 minutes] to afford the title compound (4.62 mg, 14% yield) as white solid; ¹H NMR (400 MHZ, DMSO-d₆) δ=9.97 (s, 1H), 9.17 (s, 1H), 6.99 (d, J=2.4 Hz, 1H), 6.89-6.53 (m, 3H), 5.20 (br s, 2H), 4.56-4.43 (m, 2H), 4.39-4.22 (m, 2H), 4.15-4.00 (m, 2H), 3.71 (br d, J=14.8 Hz, 1H), 3.25 (s, 3H), 3.00 (br dd, J=5.2, 9.2 Hz, 1H), 2.94 (s, 3H), 2.58-2.52 (m, 2H), 2.32 (br dd, J=4.4, 10.8 Hz, 3H), 2.05-1.55 (m, 6H), 1.30-0.93 (m, 2H), 0.73-0.46 (m, 4H); LCMS (ESI, M+1): m/z=705.4.

### Example 937

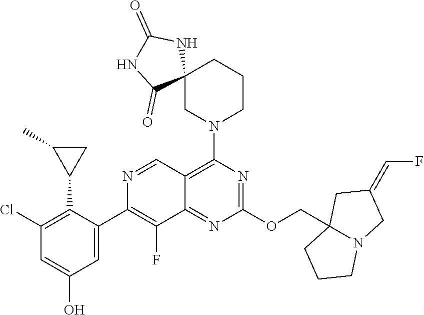 [SMILES: C[C@@H]1C[C@@H]1c1c(Cl)cc(O)cc1-c1ncc2c(N3CCC[C@]4(C3)NC(=O)NC4=O)nc(OCC34CCCN3C/C(=C\F)C4)nc2c1F]

### (5R)-7-(7-(3-chloro-5-hydroxy-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-1,3,7-triazaspiro[4.5]decane-2,4-dione

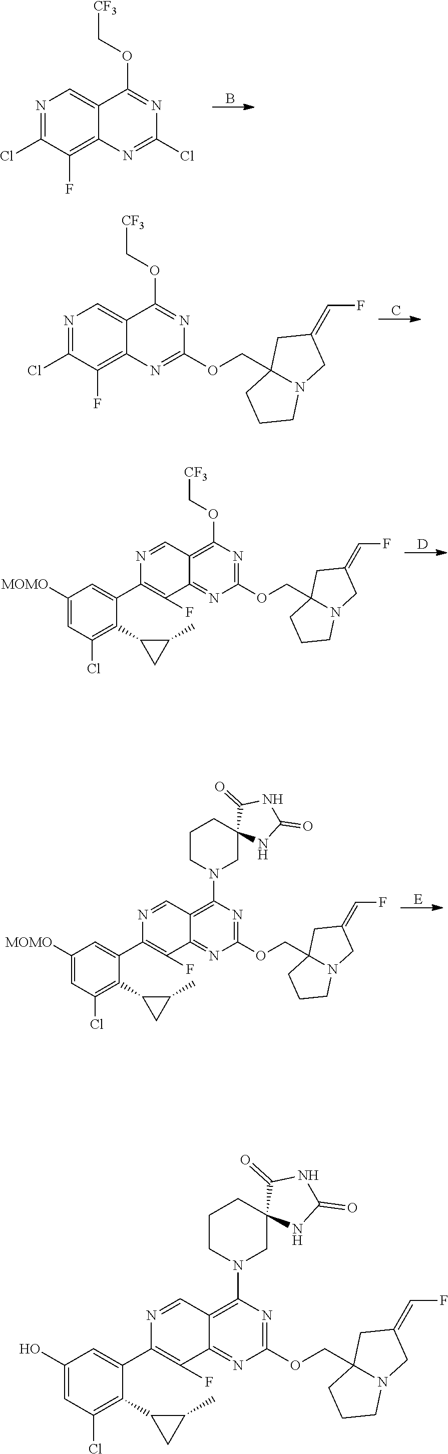

Step A. (Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (400 mg, 1.0 equiv) and (Z)-(2-(fluoromethylene)tetrahydro-1H-pyrrolizin-7a(5H)-yl)methanol (260 mg, 1.2 equiv) in THF (6 mL) were added DIEA (654 mg, 4.0 equiv) and 4 Å molecular sieves (50 mg). The reaction was stirred at 40° C. for 12 hours. The mixture was diluted with water (50 mL) and extracted with ethyl acetate (2×50 mL). The combined organic layers were washed with brine (50 mL), dried over anhydrous sodium sulfate, concentrated and purified with column chromatography [SiO₂, petroleum ether/ethyl acetate=1/0 to 5/1] to afford the title compound (410 mg, 67% yield) as yellow solid; LCMS (ESI, M+1): m/z=451.1.

Step B. 7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)-4-(2,2,2-trifluoroethoxy)pyrido[4,3-d]pyrimidine: To a solution of (Z)-7-chloro-8-fluoro-2-((2-(fluoromethylene)tetrahydro-1H-pyrrolizin-7a(5H)-yl)methoxy)-4-(2,2,2-trifluoroethoxy)pyrido[4,3-d]pyrimidine (200 mg, 1.0 equiv) and 2-(3-chloro-5-(methoxymethoxy)-2-((1S,2R)-2-methylcyclopropyl)phenyl)-4,4,5,5-tetramethyl-1,3,2-dioxaborolane (156 mg, 1.0 equiv) in methoxycyclopentane (4 mL) and water (1 mL) were added Cs₂CO₃ (434 mg, 3.0 equiv) and CataCXium A Pd G3 (64.6 mg, 0.2 equiv). The reaction was degassed and purged with N₂ for 3 times. The reaction was stirred at 100° C. for 2 hours under N₂ atmosphere. The mixture was diluted with water (30 mL) and extracted with ethyl acetate (2× 30 mL). The combined organic layers were washed with brine (30 mL), dried over anhydrous sodium sulfate, concentrated and purified with prep-TLC [SiO₂, petroleum ether/ethyl acetate=1/1] to afford the title compound (68.0 mg, 19% yield) as yellow solid; LCMS (ESI, M+1): m/z=641.3.

Step C. (5R)-7-(7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-1,3,7-triazaspiro[4.5]decane-2,4-dione: To a solution of 7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)-4-(2,2,2-trifluoroethoxy)pyrido[4,3-d]pyrimidine (68.0 mg, 1.0 equiv) and (R)-1,3,7-triazaspiro[4.5]decane-2,4-dione (26.9 mg, 1.5 equiv) in DMF (2 mL) were added DIEA (41.1 mg, 3.0 equiv) and 4 Å molecular sieves (20 mg) The reaction was stirred at 40° C. for 10 hours. The mixture was diluted with water (20 mL) and extracted with ethyl acetate (2× 20 mL). The combined organic layers were washed with brine (20 mL), dried over anhydrous sodium sulfate, concentrated and purified with prep-TLC [SiO₂, petroleum ether/ethyl acetate=1/1] to afford the title compound (44.0 mg, 47% yield) as yellow solid; LCMS (ESI, M+1): m/z=710.3.

Step D. (5R)-7-(7-(3-chloro-5-hydroxy-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-1,3,7-triazaspiro[4.5]decane-2,4-dione: To a solution of (5R)-7-(7-(3-chloro-5-(methoxymethoxy)-2-((1S,2R)-2-methylcyclopropyl)phenyl)-8-fluoro-2-(((Z)-2-(fluoromethylene)tetrahydro-1H-pyrrolizin-7a(5H)-yl)methoxy)pyrido[4,3-d]pyrimidin-4-yl)-1,3,7-triazaspiro[4.5]decane-2,4-dione (40.0 mg, 1.0 equiv) in MeOH (2 mL) was added HCl·MeOH (4M, 2 mL). The reaction was stirred at 25° C. for 2 hours. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 μm; mobile phase: water (NH₄HCO₃)-ACN]; gradient: 35%-65% B over 9 minutes] to afford the title compound (4.32 mg, 11% yield) as white solid, ¹H NMR (400 MHZ, METHANOL-d4) δ=9.11-9.06 (m, 1H), 7.00 (d, J=2.8 Hz, 1H), 6.83 (d, J=2.8 Hz, 1H), 6.75-6.50 (m, 1H), 4.58 (br t, J=12.4 Hz, 1H), 4.44 (br d, J=13.6 Hz, 1H), 4.37-4.20 (m, 2H), 3.86-3.66 (m, 3H), 3.45 (br d, J=14.8 Hz, 1H), 3.18-3.11 (m, 1H), 2.78-2.65 (m, 2H), 2.44 (br d, J=15.6 Hz, 1H), 2.31-2.18 (m, 1H), 2.17-1.77 (m, 9H), 1.19-1.04 (m, 1H), 0.64 (br d, J=5.6 Hz, 4H); LCMS (ESI, M+1): m/z=666.3.

### Example 938

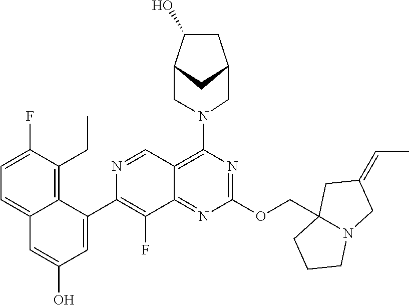 [SMILES: C/C=C1\CN2CCCC2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-2-(((Z)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol

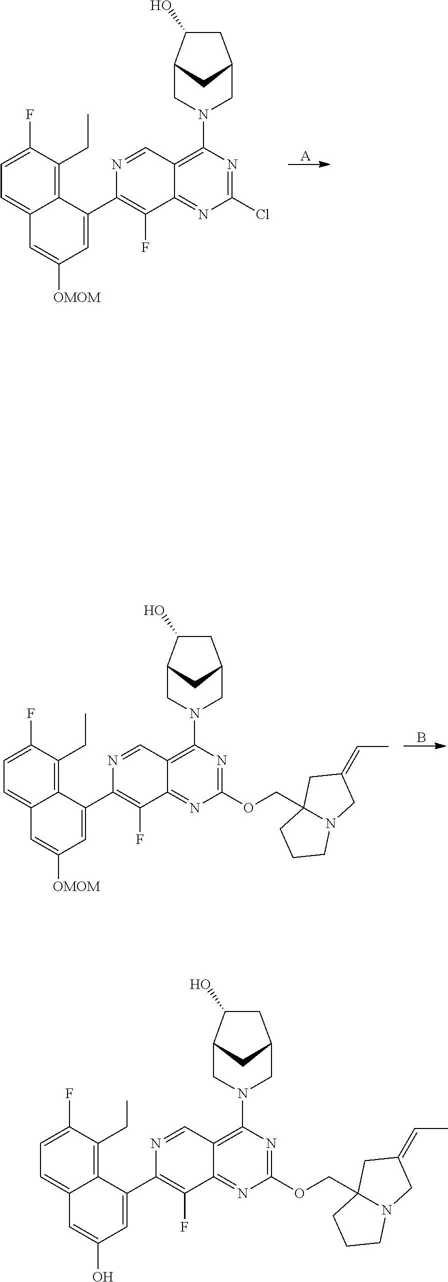

Step A. (1R,5R,6R)-3-(7-(8-ethyl-7-fluoro-3-(methoxymethoxy)naphthalen-1-yl)-2-(((Z)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (120 mg, 1.0 equiv) and (Z)-(2-ethylidenetetrahydro-1H-pyrrolizin-7a(5H)-yl)methanol (55.6 mg, 1.5 equiv) in DMF (0.5 mL) and THF (0.5 mL) were added Cs₂CO₃ (217 mg, 3.0 equiv) and DABCO (24.9 mg, 1.0 equiv). The reaction was stirred at 25° C. for 3 hours. The mixture was diluted with water (10 mL) and extracted with ethyl acetate (2×10 mL). The combined organic layers were washed with brine (10 mL), dried over anhydrous sodium sulfate, concentrated, and purified with reversed phase flash chromatography [C18, 0.1% formic acid condition] to afford the title compound (35.0 mg, 21% yield) as yellow solid; LCMS (ESI, M+1): m/z=672.4.

Step B. (1R,5R,6R)-3-(7-(8-ethyl-7-fluoro-3-hydroxynaphthalen-1-yl)-2-(((Z)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol: solution To a of (1R,5R,6R)-3-(7-(8-ethyl-7-fluoro-3-(methoxymethoxy)naphthalen-1-yl)-2-(((Z)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol (30.0 mg, 1.0 equiv) in DCM (0.1 mL) was added TFA (307 mg, 60 equiv) at 0° C. The reaction was stirred at 0° C. for 0.5 hours. The mixture was adjusted to pH=7 with saturated NaHCO₃ (10 mL) and extracted with DCM (2×10 mL). The combined organic layers were dried over anhydrous sodium sulfate, concentrated, and purified by prep-HPLC [column: Phenomenex luna C18 150×25 mm×10 μm; A: water (FA), B: ACN, B %: 19%-49% over 10 min] to afford the title compound (10.9 mg, 36% yield, 0.49 HCOOH) as white solid; ¹H NMR (400 MHZ, METHANOL-d4) δ=9.30-9.19 (m, 1H), 7.68 (dd, J=6.0, 8.8 Hz, 1H), 7.36-7.19 (m, 2H), 7.06 (dd, J=2.4, 15.6 Hz, 1H), 5.58-5.51 (m, 1H), 4.80-4.74 (m, 1H), 4.52-4.41 (m, 2H), 4.33 (qd, J=5.2, 10.8 Hz, 1H), 4.10-3.96 (m, 1H), 3.81 (br dd, J=12.4, 20.0 Hz, 1H), 3.65 (br d, J=13.6 Hz, 1H), 3.56-3.41 (m, 2H), 3.07-2.92 (m, 1H), 2.86 (br d, J=14.0 Hz, 1H), 2.60 (br d, J=16.0 Hz, 1H), 2.54-2.36 (m, 2H), 2.34-1.88 (m, 9H), 1.86-1.76 (m, 1H), 1.66 (br d, J=6.8 Hz, 3H), 1.44-1.33 (m, 1H), 0.79-0.79 (m, 1H), 0.80 (q, J=7.2 Hz, 2H); LCMS (ESI, M−1): m/z=628.3.

### Example 939

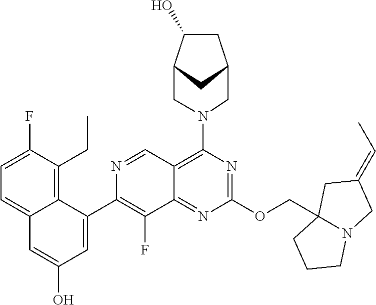 [SMILES: C/C=C1/CN2CCCC2(COc2nc(N3C[C@@H]4C[C@H](C3)[C@H](O)C4)c3cnc(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-2-(((E)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol

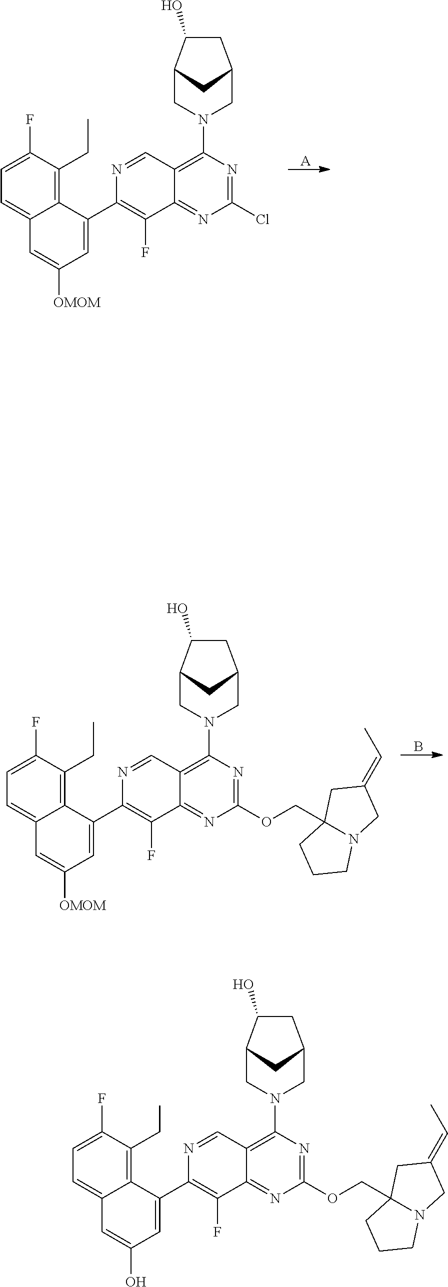

Step A. (1R,5R,6R)-3-(7-(8-ethyl-7-fluoro-3-(methoxymethoxy)naphthalen-1-yl)-2-(((E)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol: To a solution of (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (150 mg, 1.0 equiv) and (E)-(2-ethylidenetetrahydro-1H-pyrrolizin-7a(5H)-yl)methanol (69.6 mg, 1.5 equiv) in THF (0.5 mL) and DMF (0.5 mL) were added DABCO (31.1 mg, 1.0 equiv) and Cs₂CO₃ (271 mg, 3.0 equiv). The reaction was stirred at 20° C. for 12 hours. The mixture was diluted with water (5.0 mL) and extracted with EtOAc (3×5 mL). The combined organic layers were washed with brine (20 mL), dried over anhydrous sodium sulfate, concentrated and purified with reversed phase flash chromatography [C18, 0.1% formic acid condition] to afford the title compound (70 mg, 32% yield) as yellow solid; LCMS (ESI, M+1): m/z=672.4.

Step B. (1R,5R,6R)-3-(7-(8-ethyl-7-fluoro-3-hydroxynaphthalen-1-yl)-2-(((E)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-2-(((E)-2-ethylidenetetrahydro-1H-pyrrolizin-7a(5H)-yl)methoxy)-8-fluoropyrido[4,3-d]pyrimidin-4-yl)-3-azabicyclo[3.2.1]octan-6-ol (65.0 mg, 1.0 equiv) in MeOH (0.3 mL) was added HCl·MeOH (4 M, 1.3 mL). The reaction was stirred at 0° C. for 0.5 hours. The mixture was adjusted to pH=8 with saturated NaHCO₃ aqueous (5.0 mL) at 0° C. and extracted with DCM (3×5 mL). The combined organic layers were washed with brine (10 mL), dried over anhydrous sodium sulfate, concentrated, and purified with prep-HPLC [Phenomenex luna C18 150×25 mm×10 μm; A: water (FA), B: ACN; B %: 18%-48% over 10 min] and lyophilized to afford the title compound (14.0 mg, 23% yield, 0.25 HCOOH) as white solid; ¹H NMR (400 MHZ, METHANOL-d₄) δ=9.32-9.21 (m, 1H), 7.70-7.66 (m, 1H), 7.31 (d, J=2.4 Hz, 1H), 7.25 (br t, J=9.2 Hz, 1H), 7.06 (dd, J=2.4, 15.6 Hz, 1H), 5.73-5.58 (m, 1H), 4.62-4.54 (m, 4H), 4.36-4.32 (m, 1H), 4.21-4.12 (m, 1H), 3.91-3.66 (m, 2H), 3.65-3.42 (m, 2H), 3.16-3.04 (m, 1H), 2.90 (br d, J=16.8 Hz, 1H), 2.70 (br d, J=16.0 Hz, 1H), 2.54-2.39 (m, 2H), 2.36-2.33 (m, 1H), 2.31-2.21 (m, 2H), 2.20 (br s, 4H), 1.98-1.90 (m, 1H), 1.87-1.77 (m, 1H), 1.70 (br s, 3H), 1.38 (br dd, J=2.0, 14.4 Hz, 1H), 0.80 (q, J=7.2 Hz, 3H); LCMS (ESI, M+1): m/z=628.4.

### Example 940

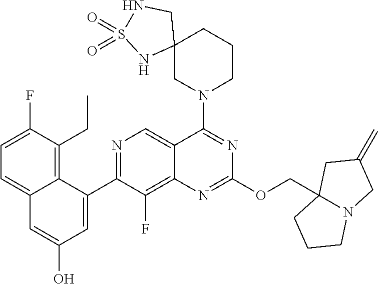 [SMILES: C=C1CN2CCCC2(COc2nc(N3CCCC4(CNS(=O)(=O)N4)C3)c3cnc(-c4cc(O)cc5ccc(F)c(CC)c45)c(F)c3n2)C1]

### 7-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-2-thia-1,3,7-triazaspiro[4.5]decane

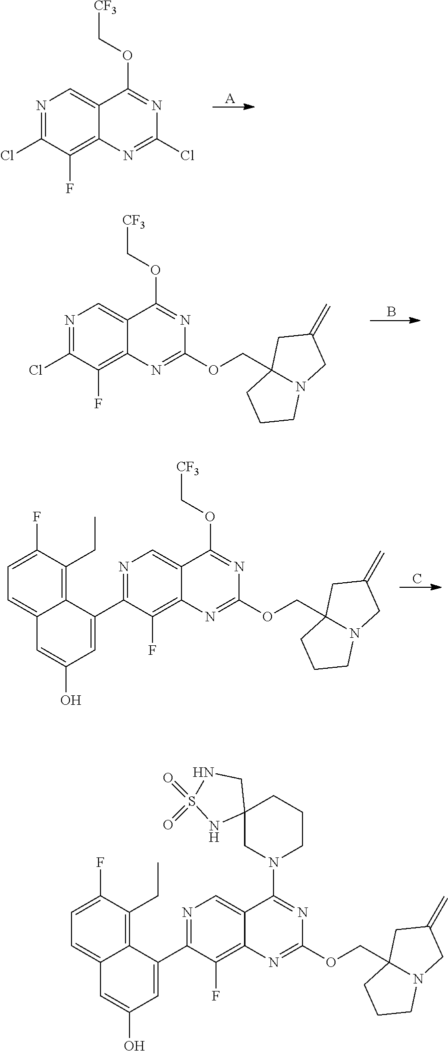

Step A. 7-chloro-8-fluoro-2-((2-methylenetetrahydro-1H-pyrrolizin-7a(5H)-yl)methoxy)-4-(2,2,2-trifluoroethoxy)pyrido[4,3-d]pyrimidine: To a solution of 2,7-dichloro-8-fluoro-4-(2,2,2-trifluoroethoxy)pyrido[4,3-d]pyrimidine (4.00 g, 1.0 equiv) and (2-methylenetetrahydro-1H-pyrrolizin-7a(5H)-yl)methanol (2.09 g, 1.2 equiv) in THF (20 mL) were added DIEA (5.89 g, 4.0 equiv) and 4 Å molecular sieves (500 mg). The reaction was stirred at 40° C. for 10 hours. The mixture was diluted with water (50 mL) and extracted with ethyl acetate (2× 50 mL). The combined organic layers were washed with brine (50 mL), dried over anhydrous sodium sulfate, concentrated and purified with column chromatography [SiO₂, petroleum ether/ethyl acetate=1/0 to 5/1] to afford the title compound (3.00 g, 50% yield) as yellow solid; LCMS (ESI, M+1): m/z=433.1.

Step B. 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol: To a solution of 7-chloro-8-fluoro-2-((2-methylenetetrahydro-1H-pyrrolizin-7a(5H)-yl)methoxy)-4-(2,2,2-trifluoroethoxy)pyrido[4,3-d]pyrimidine (3.00 g, 1.0 equiv) and 5-ethyl-6-fluoro-4-(4,4,5,5-tetramethyl-1,3,2-dioxaborolan-2-yl)naphthalen-2-ol (2.19 g, 1.0 equiv) in methoxycyclopentane (20 mL) and water (5 mL) were added Cs₂CO₃ (6.78 g, 3.0 equiv) and CataCXium A Pd G3 (1.01 g, 0.2 equiv). The reaction was degassed and purged with N₂ for 3 times. The reaction was stirred at 100° C. for 2 hours under N₂ atmosphere. The mixture was diluted with water (100 mL) and extracted with ethyl acetate (2×100 mL). The combined organic layers were washed with brine (50 mL), dried over anhydrous sodium sulfate, concentrated and purified with prep-HPLC [column: C18 150×30 mm; mobile phase: water (FA)-ACN; gradient: 30%-60% B over 7 minutes] to afford the title compound (500 mg, 9.2% yield) as yellow solid; LCMS (ESI, M+1): m/z=587.2.

Step C. 7-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-2-thia-1,3,7-triazaspiro[4.5]decane 2,2-dioxide: To a solution of 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol (50.0 mg, 1.0 equiv) and 2-thia-1,3,7-triazaspiro[4.5]decane 2,2-dioxide (24.5 mg, 1.5 equiv) in DMF (1 mL) were added 4 Å molecular sieve (20.0 mg) and K₃PO₄ (54.3 mg, 3.0 equiv). The reaction was stirred at 60° C. for 4 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge 150×25 mm×5 um; mobile phase: water (NH₄HCO₃)-ACN; gradient: 38%-68% B over 9 minutes] to afford the title compound (30.5 mg, 50% yield) as white solid; ¹H NMR (400 MHZ, METHANOL-d₄) δ=9.09 (s, 1H), 7.67 (ddd, J=3.2, 5.6, 8.8 Hz, 1H), 7.31-7.21 (m, 2H), 7.04 (dd, J=2.8, 4.8 Hz, 1H), 4.98 (br s, 2H), 4.59-4.50 (m, 1H), 4.41-4.25 (m, 3H), 3.87-3.72 (m, 2H), 3.68-3.59 (m, 1H), 3.40-3.35 (m, 1H), 3.21-3.14 (m, 2H), 2.83-2.68 (m, 2H), 2.55-2.44 (m, 2H), 2.21-1.81 (m, 10H), 0.81 (dt, J=4.0, 7.2 Hz, 3H); LCMS (ESI, M+1): m/z=678.3.

### Example 941

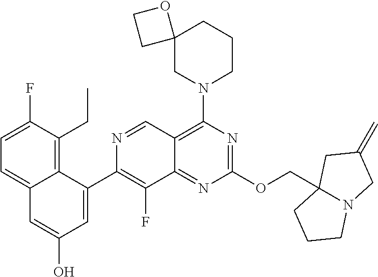 [SMILES: C=C1CN2CCCC2(COc2nc(N3CCCC4(CCO4)C3)c3cnc(-c4cc(O)cc5ccc(F)c(CC)c45)c(F)c3n2)C1]

### 5-ethyl-6-fluoro-4-(8-fluoro-2-((2-methylenetetrahydro-1H-pyrrolizin-7a(5H)-yl)methoxy)-4-(1-oxa-6-azaspiro[3.5]nonan-6-yl)pyrido[4,3-d]pyrimidin-7-yl)naphthalen-2-ol

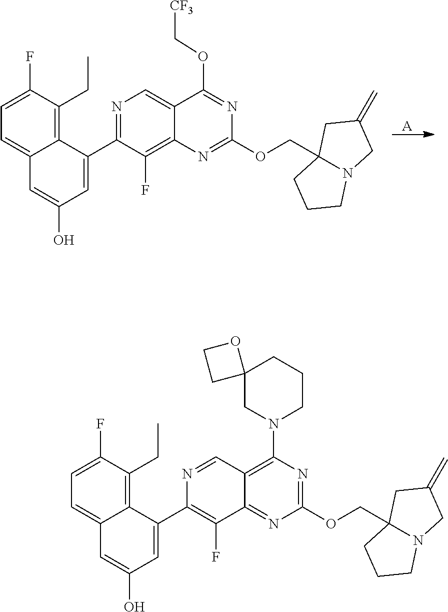

Step A. 5-ethyl-6-fluoro-4-(8-fluoro-2-((2-methylenetetrahydro-1H-pyrrolizin-7a(5H)-yl)methoxy)-4-(1-oxa-6-azaspiro[3.5]nonan-6-yl)pyrido[4,3-d]pyrimidin-7-yl)naphthalen-2-ol: To a solution of 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol (45.0 mg, 1.0 equiv) in DMF (0.5 mL) were added 1-oxa-8-azaspiro[3.5]nonane (20.3 mg, 0.8 equiv, oxalate), 4 Å molecular sieve (30.0 mg) and K₃PO₄ (50.0 mg, 3.0 equiv). The reaction was stirred at 60° C. for 3 hours. The mixture was diluted with water (4 mL) and extracted with ethyl acetate (3×5 mL). The combined organic layers were washed with brine (10 mL), dried over anhydrous sodium sulfate, concentrated and purified with prep-HPLC [column: Waters Xbridge 150×25 mm×5 um; mobile phase: water (NH₄HCO₃)-ACN; gradient: 45%-75% B over 9 minutes] to afford the title compound (4.38 mg, 8.8% yield) as white solid; 1HNMR (400 MHZ, METHANOL-d₄) δ=9.26 (d, J=5.2 Hz, 1H), 7.68 (dd, J=6.0, 9.2 Hz, 1H), 7.31 (d, J=2.8 Hz, 1H), 7.25 (t, J=9.6 Hz, 1H), 7.08 (d, J=2.4 Hz, 1H), 4.98 (s, 2H), 4.70-4.52 (m, 4H), 4.50-4.38 (m, 1H), 4.36-4.26 (m, 2H), 3.90-3.71 (m, 2H), 3.54-3.41 (m, 1H), 3.18-3.11 (m, 1H), 2.83-2.68 (m, 2H), 2.57-2.44 (m, 4H), 2.33-2.24 (m, 1H), 2.23-2.12 (m, 2H), 2.06-1.79 (m, 6H), 0.86-0.79 (m, 3H); LCMS (ESI, M+1): m/z=614.3.

### Example 942

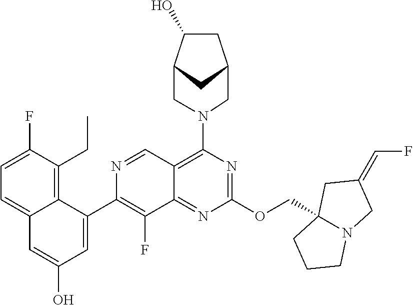 [SMILES: CCc1c(F)ccc2cc(O)cc(-c3ncc4c(N5C[C@@H]6C[C@H](C5)[C@H](O)C6)nc(OC[C@@]56CCCN5C/C(=C\F)C6)nc4c3F)c12]

### (1R,5R,6R)-3-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol

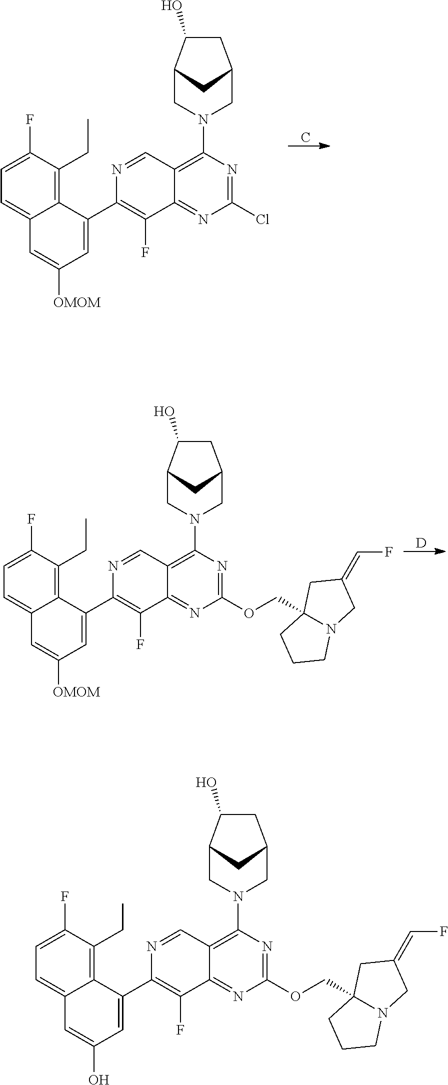

Step A. (1R,5R,6R)-3-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol: solution of To a (S,Z)-(2-(fluoromethylene)tetrahydro-1H-pyrrolizin-7a(5H)-yl)methanol (31.6 mg, 2.0 equiv) in THF (2.0 mL) was added NaH (11.1 mg, 60% purity, 3.0 equiv) at 0° C. After stirring for 0.5 hours, (1R,5R,6R)-3-(2-chloro-7-(8-ethyl-7-fluoro-3-(methoxymethoxy)naphthalen-1-yl)-8-fluoropyrido[4,3-d]pyrimidin-4-yl)-3-azabicyclo[3.2.1]octan-6-ol (50 mg, 1.0 equiv) was added. The reaction was stirred at 0° C. for 2 hours. The mixture was quenched by NH₄Cl solution (10 mL) and extracted by ethyl acetate (3×10 mL). The combined organic layers were dried by Na₂SO₄, filtered, concentrated and purified by reversed phase flash chromatography [C18, 0.1% formic acid condition] to afford the title compound (190 mg, 48% yield) as yellow solid; LCMS (ESI, M+1): m/z=676.4.

Step B. (1R,5R,6R)-3-(7-(8-ethyl-7-fluoro-3-hydroxynaphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol: To a solution of (1R,5R,6R)-3-(7-(8-ethyl-7-fluoro-3-(methoxymethoxy)naphthalen-1-yl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-azabicyclo[3.2.1]octan-6-ol (80.0 mg, 1.0 equiv) in DCM (2.0 mL) was added TFA (0.60 mL, 68 equiv). The reaction was stirred at 0° C. for 2 hours. The mixture was diluted with acetonitrile, adjusted to pH=8 with NaHCO₃ aqueous solution, filtered, concentrated and purified by prep-HPLC [Daisogel SP ODS RPS 150×25 mm×5 um; A: water (NH₄HCO₃)-ACN; B: CAN, 44%-74% over 10 min] to afford the title compound (54.0 mg, 71% yield) as white solid; ¹H NMR (400 MHZ, METHANOL-d₄) δ=9.30-9.16 (m, 1H), 7.67 (dd, J=5.8, 8.8 Hz, 1H), 7.32-7.17 (m, 2H), 7.06 (dd, J=2.8, 14.7 Hz, 1H), 6.80-6.49 (m, 1H), 5.07-4.89 (m, 1H), 4.82-4.72 (m, 2H), 4.37-4.28 (m, 2H), 4.27-4.21 (m, 1H), 3.89-3.72 (m, 2H), 3.53-3.38 (m, 2H), 3.21-3.10 (m, 1H), 2.78-2.67 (m, 2H), 2.53-2.37 (m, 3H), 2.29-2.19 (m, 2H), 2.18-2.07 (m, 2H), 2.04-1.96 (m, 1H), 1.94-1.86 (m, 2H), 1.85-1.77 (m, 1H), 1.48-1.37 (m, 1H), 0.80 (q, J=7.4 Hz, 3H); LCMS (ESI, M+1): m/z=632.3.

### Example 943

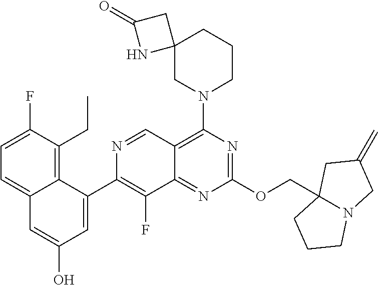 [SMILES: C=C1CN2CCCC2(COc2nc(N3CCCC4(CC(=O)N4)C3)c3cnc(-c4cc(O)cc5ccc(F)c(CC)c45)c(F)c3n2)C1]

### 6-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-1,6-diazaspiro[3.5]nonan-2-one

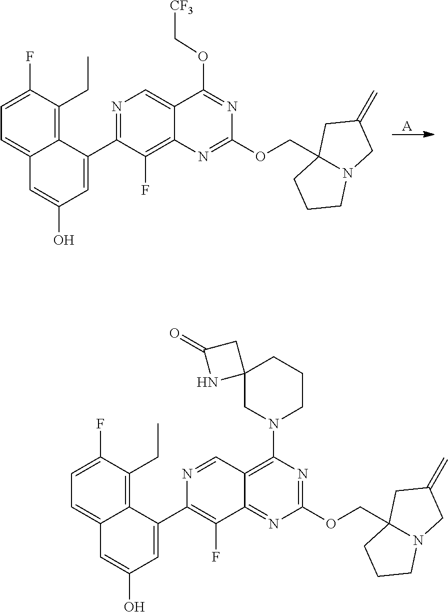

Step A. 6-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)-1,6-diazaspiro[3.5]nonan-2-one: To a solution of 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol (40.0 mg, 1.0 equiv) and 1,8-diazaspiro[3.5]nonan-2-one (14.3 mg, 1.5 equiv) in DMF (1 mL) were added 4 Å molecular sieve (10.0 mg) and K₃PO₄ (43.4 mg, 3.0 equiv). The reaction was stirred at 60° C. for 2 hours. The mixture was diluted with water (10 mL) and extracted with ethyl acetate (2×10 mL). The combined organic layers were washed with brine (2×10 mL), dried over anhydrous sodium sulfate, concentrated and purified with prep-HPLC [column: Waters Xbridge 150×25 mm×5 um; mobile phase: water (NH₄HCO₃)-ACN; gradient: 35%-65% B over 9 minutes] to afford the title compound (3.00 mg, 6.9% yield) as white solid; ¹H NMR (400 MHZ, DMSO-d₆) δ=9.93 (s, 1H), 9.08 (d, J=2.0 Hz, 1H), 8.56 (br s, 1H), 7.77 (dd, J=6.0, 9.2 Hz, 1H), 7.38-7.31 (m, 2H), 7.03 (t, J=2.8 Hz, 1H), 4.89 (br s, 2H), 4.32-3.98 (m, 4H), 3.94-3.76 (m, 1H), 3.70-3.49 (m, 2H), 3.18 (br d, J=14.0 Hz, 1H), 3.04-2.94 (m, 1H), 2.86-2.73 (m, 1H), 2.69-2.55 (m, 3H), 2.35 (br d, J=15.6 Hz, 2H), 2.19-2.05 (m, 1H), 2.03-1.93 (m, 2H), 1.91-1.64 (m, 6H), 0.73 (t, J=7.2 Hz, 3H); LCMS (ESI, M+1): m/z=627.3.

### Example 944

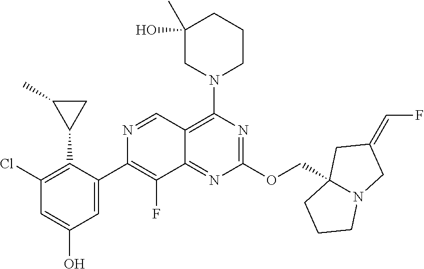 [SMILES: C[C@@H]1C[C@@H]1c1c(Cl)cc(O)cc1-c1ncc2c(N3CCC[C@@](C)(O)C3)nc(OC[C@@]34CCCN3C/C(=C\F)C4)nc2c1F]

### (R)-1-(7-(3-chloro-5-hydroxy-2-((1S,2R)-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol

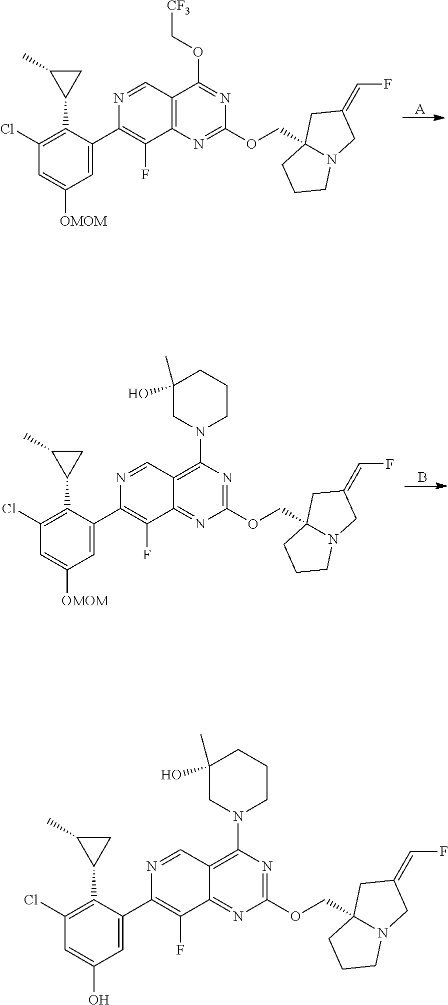

Step A. (R)-1-(7-(3-chloro-5-(methoxymethoxy)-2-((1S,2R)-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol: To a solution of 7-(3-chloro-5-(methoxymethoxy)-2-((1S,2R)-2-methylcyclopropyl)phenyl)-8-fluoro-2-(((S,Z)-2-(fluoromethylene)tetrahydro-1H-pyrrolizin-7a(5H)-yl)methoxy)-4-(2,2,2-trifluoroethoxy)pyrido[4,3-d]pyrimidine (70.0 mg, 1.0 equiv) and (R)-3-methylpiperidin-3-ol (18.9 mg, 1.1 equiv, HCl) in DMF (1 mL) were added DIEA (70.6 mg, 5.0 equiv) and 4 Å molecular sieve (30 mg). The reaction was stirred at 40° C. for 10 hours. The mixture was diluted with water (10 mL) and extracted with ethyl acetate (2×10 mL). The combined organic layers were washed with brine (10 mL), dried over anhydrous sodium sulfate, concentrated, and purified with prep-TLC [SiO₂, DCM/MeOH=10/1] to afford the title compound (75.0 mg, 98% yield) as white solid; LCMS (ESI, M+1): m/z=656.3.

Step B. (R)-1-(7-(3-chloro-5-hydroxy-2-((1S,2R)-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol: To a solution of (R)-1-(7-(3-chloro-5-(methoxymethoxy)-2-((1S,2R)-2-methylcyclopropyl)phenyl)-8-fluoro-2-(((S,Z)-2-(fluoromethylene)tetrahydro-1H-pyrrolizin-7a(5H)-yl)methoxy)pyrido[4,3-d]pyrimidin-4-yl)-3-methylpiperidin-3-ol (70.0 mg, 1.0 equiv) in MeCN (2 mL) was added HCl·dioxane (4M, 2 mL). The reaction was stirred at 0° C. for 1 hour. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 um; mobile phase: water (NH₄HCO₃)-ACN; gradient: 45%-75% B over 9 minutes] to afford the title compound (30.5 mg, 46% yield) as white solid, ¹H NMR (400 MHZ, METHANOL-d₄) δ=9.19 (d, J=13.6 Hz, 1H), 7.00 (d, J=2.4 Hz, 1H), 6.82 (br s, 1H), 6.78-6.50 (m, 1H), 4.60-4.45 (m, 1H), 4.37-4.22 (m, 3H), 3.84 (br d, J=14.8 Hz, 1H), 3.63-3.57 (m, 1H), 3.49-3.40 (m, 2H), 3.19-3.12 (m, 1H), 2.77-2.66 (m, 2H), 2.44 (br d, J=16.0 Hz, 1H), 2.21-1.72 (m, 10H), 1.28 (d, J=2.0 Hz, 3H), 1.19-1.06 (m, 1H), 0.66 (br d, J=5.6 Hz, 4H); LCMS (ESI, M+1): m/z=612.3.

### Example 945

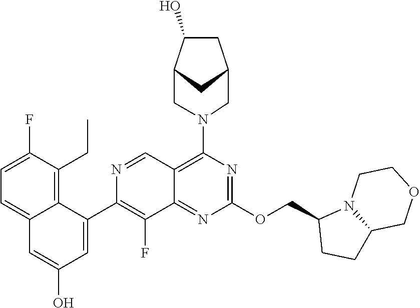 [SMILES: CCc1c(F)ccc2cc(O)cc(-c3ncc4c(N5C[C@@H]6C[C@H](C5)[C@H](O)C6)nc(OC[C@@H]5CC[C@H]6COCCN65)nc4c3F)c12]

### (1R,5R,6R)-3-(7-(8-ethyl-7-fluoro-3-hydroxynaphthalen-1-yl)-8-fluoro-2-(((6S,8aS)-hexahydro-1H-pyrrolo[2,1-c][1,4]oxazin-6-yl)methoxy)pyrido[4,3-d]pyrimidin-4-yl)-3-azabicyclo[3.2.1]octan-6-ol

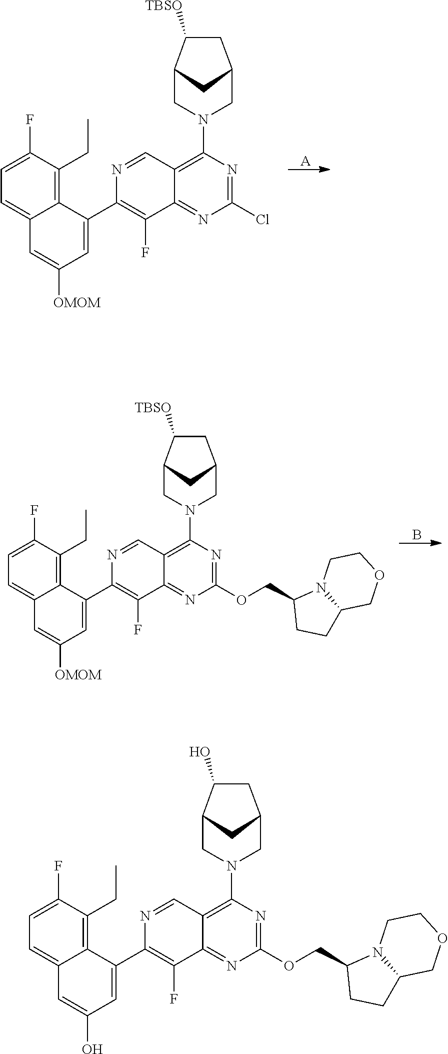

Step A. (6S,8aS)-6-(((4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-8-fluoropyrido[4,3-d]pyrimidin-2-yl)oxy)methyl) hexahydro-1H-pyrrolo[2,1-c][1,4]oxazine: To a solution of 4-((1R,5R,6R)-6-((tert-butyldimethylsilyl)oxy)-3-azabicyclo[3.2.1]octan-3-yl)-2-chloro-7-(8-ethyl-7-fluoro-3-(methoxymethoxy)naphthalen-1-yl)-8-fluoropyrido[4,3-d]pyrimidine (50.0 mg, 1.0 equiv) in THF (1.00 mL) was added NaH (11.1 mg, 60% purity, 3.0 equiv) at 0° C. The reaction was stirred at 0° C. for 10 minutes. ((6S,8aS)-hexahydro-1H-pyrrolo[2,1-c][1,4]oxazin-6-yl)methanol (22 mg, 1.5 equiv) in THF (1.00 mL) was added. The reaction was stirred at 0° C.-20° C. for 3 hours. The mixture was diluted with water (10 mL) and extracted with ethyl acetate (2×5 mL). The combined organic layers were dried over anhydrous sodium sulfate and concentrated under vacuum to afford the title compound (35.0 mg, crude) as yellow oil; LCMS (ESI, 1/2M+1, M+1): m/z=388.8, 776.4.

Step B. (1R,5R,6R)-3-(7-(8-ethyl-7-fluoro-3-hydroxynaphthalen-1-yl)-8-fluoro-2-(((6S,8aS)-hexahydro-1H-pyrrolo[2,1-c][1,4]oxazin-6-yl)methoxy)pyrido[4,3-d]pyrimidin-4-yl)-3-azabicyclo[3.2.1]octan-6-ol: To a solution of (6S,8aS)-6-(((4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-8-fluoropyrido[4,3-d]pyrimidin-2-yl)oxy)methyl) hexahydro-1H-pyrrolo[2,1-c][1,4]oxazine (25.0 mg, 1.0 equiv) in MeOH (2.00 mL) was added HCl·MeOH (4 M, 2.00 mL) at 0° C. The reaction was stirred at 0-20° C. for 3 hours. The mixture was concentrated, basified by saturated NaHCO₃ solution (20 mL) and extracted with ethyl acetate (2×10 mL) The combined organic layers were washed with brine (30 mL), dried over anhydrous sodium sulfate, concentrated and purified with prep-HPLC [column: Waters Xbridge 150×25 mm×5 μm; A: water (NH₄HCO₃), B: ACN; B %: 35%-65% B over 9 min] to afford the title compound (10.2 mg, 50% yield) as white solid; ¹H NMR (400 MHZ, METHANOL-d4) δ=9.30-9.18 (m, 1H), 7.67 (dd, J=6.0, 8.8 Hz, 1H), 7.30 (d, J=2.8 Hz, 1H), 7.24 (dt, J=2.0, 9.6 Hz, 1H), 7.10-7.02 (m, 1H), 5.03-4.89 (m, 1H), 4.82-4.74 (m, 1H), 4.62-4.46 (m, 1H), 4.42-4.27 (m, 2H), 3.86-3.61 (m, 5H), 3.54-3.42 (m, 1H), 3.39-3.34 (m, 1H), 3.20-2.97 (m, 3H), 2.57-2.36 (m, 2H), 2.31-2.06 (m, 4H), 1.99-1.86 (m, 2H), 1.84-1.68 (m, 2H), 1.54-1.37 (m, 2H), 0.80 (q, J=7.2 Hz, 3H); LCMS (ESI, M+1): m/z=618.3.

### Example 946

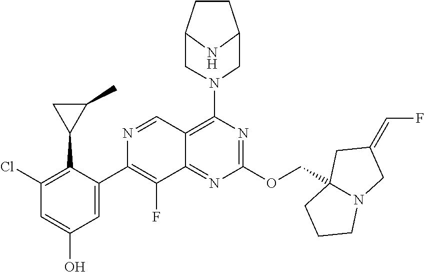

### 3-(4-((1R,5S)-3,8-diazabicyclo[3.2.1]octan-3-yl)-8-fluoro-2-(((S,Z)-2-(fluoromethylene)tetrahydro-1H-pyrrolizin-7a(5H)-yl)methoxy)pyrido[4,3-d]pyrimidin-7-yl)-5-chloro-4-((1S,2R)-2-methylcyclopropyl)phenol

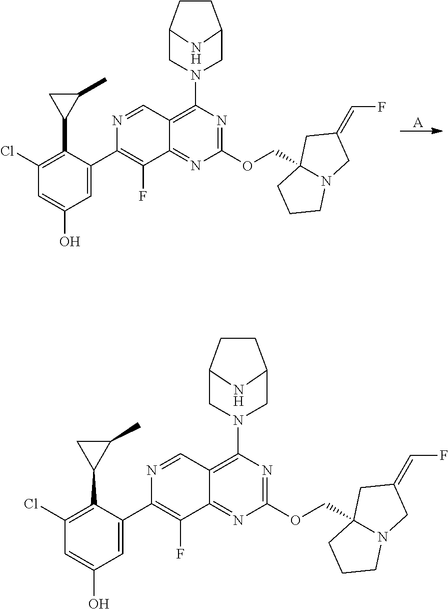

Step A. 3-(4-((1R,5S)-3,8-diazabicyclo[3.2.1]octan-3-yl)-8-fluoro-2-(((S,Z)-2-(fluoromethylene)tetrahydro-1H-pyrrolizin-7a(5H)-yl)methoxy)pyrido[4,3-d]pyrimidin-7-yl)-5-chloro-4-((1S,2R)-2-methylcyclopropyl)phenol: 3-(4-((1R,5S)-3,8-diazabicyclo[3.2.1]octan-3-yl)-8-fluoro-2-(((S,Z)-2-(fluoromethylene)tetrahydro-1H-pyrrolizin-7a(5H)-yl)methoxy)pyrido[4,3-d]pyrimidin-7-yl)-5-chloro-4-((1R,2S)-2-methylcyclopropyl)phenol (200 mg) was purified with SFC [column: DAICEL CHIRALPAK IC 250 mm×30 mm, 10 um; mobile phase: CO₂-ACN/i-PrOH (0.1% NH₃H₂O); B %: 50%, isocratic elution mode] to afford the title compound (58.8 mg, 29% yield) as white solid; ¹H NMR (400 MHZ, METHANOL-d₄) δ=9.04 (s, 1H), 7.00 (d, J=2.6 Hz, 1H), 6.81 (d, J=2.4 Hz, 1H), 6.77-6.51 (m, 1H), 4.67-4.53 (m, 2H), 4.36-4.20 (m, 2H), 3.85 (br d, J=14.8 Hz, 1H), 3.75-3.64 (m, 4H), 3.47 (br d, J=14.8 Hz, 1H), 3.22-3.11 (m, 1H), 2.78-2.66 (m, 2H), 2.44 (br d, J=15.2 Hz, 1H), 2.20-2.09 (m, 1H), 2.03-1.72 (m, 9H), 1.19-1.02 (m, 1H), 0.65 (br d, J=5.6 Hz, 4H); LCMS (ESI, M+1): m/z=609.2.

### Example 947

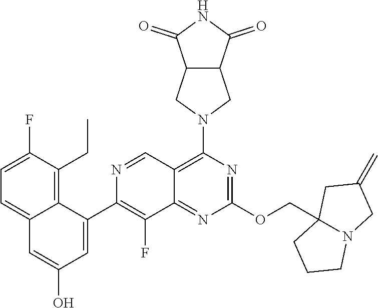 [SMILES: C=C1CN2CCCC2(COc2nc(N3CC4C(=O)NC(=O)C4C3)c3cnc(-c4cc(O)cc5ccc(F)c(CC)c45)c(F)c3n2)C1]

### 5-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)tetrahydropyrrolo[3,4-c]pyrrole-1,3(2H,3aH)-dione

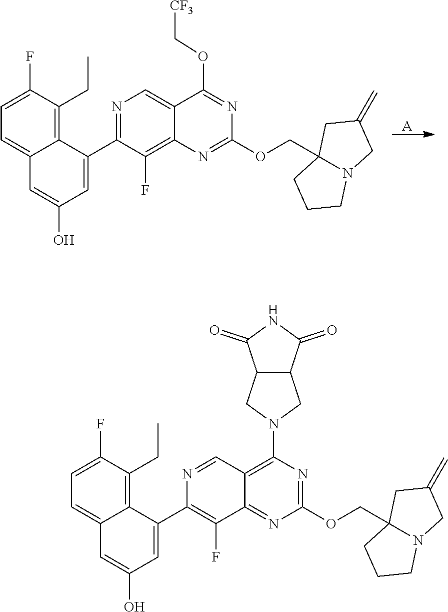

Step A. 5-(7-(8-ethyl-7-fluoro-3-hydroxynaphthalen-1-yl)-8-fluoro-2-((2-methylenetetrahydro-1H-pyrrolizin-7a(5H)-yl)methoxy)pyrido[4,3-d]pyrimidin-4-yl)tetrahydropyrrolo[3,4-c]pyrrole-1,3(2H,3aH)-dione: To a solution of 5-ethyl-6-fluoro-4-(8-fluoro-2-((2-methylenetetrahydro-1H-pyrrolizin-7a(5H)-yl)methoxy)-4-(2,2,2-trifluoroethoxy)pyrido[4,3-d]pyrimidin-7-yl)naphthalen-2-ol (50.0 mg, 1.0 equiv) and tetrahydropyrrolo[3,4-c]pyrrole-1,3(2H,3aH)-dione (17.9 mg, 1.5 equiv) in DMF (1 mL) were added 4 Å molecular sieve (20 mg) and K₃PO₄ (54.3 mg, 3.0 equiv). The reaction was stirred at 60° C. for 4 hours. The mixture was diluted with water (10 mL) and extracted with ethyl acetate (2×10 mL). The combined organic layers were washed with brine (10 mL), dried over anhydrous sodium sulfate, concentrated and purified with prep-HPLC [column: Waters Xbridge 150×25 mm×5 μm; mobile phase: water (NH₄HCO₃)-ACN; gradient: 30%-60% B over 9 minutes] to afford the title compound (13.1 mg, 22% yield) as yellow gum; ¹H NMR (400 MHZ, DMSO-d₆) δ=11.76-10.98 (m, 1H), 10.15-9.83 (m, 1H), 9.26 (s, 1H), 7.76 (dd, J=6.0, 9.2 Hz, 1H), 7.46-7.25 (m, 2H), 7.02 (d, J=2.4 Hz, 1H), 4.90 (br s, 2H), 4.41 (br d, J=11.6 Hz, 2H), 4.28 (br d, J=2.8 Hz, 2H), 4.14-4.00 (m, 2H), 3.76-3.65 (m, 2H), 3.56 (br d, J=14.0 Hz, 1H), 3.19 (br d, J=14.0 Hz, 1H), 3.06-2.95 (m, 1H), 2.62-2.54 (m, 2H), 2.41-2.26 (m, 2H), 2.20-2.05 (m, 1H), 1.99-1.94 (m, 1H), 1.89-1.75 (m, 2H), 1.72-1.63 (m, 1H), 0.79-0.66 (m, 3H); LCMS (ESI, M+1): m/z=627.3.

### Example A

### KRas Binding Assay

This Example illustrates that exemplary compounds of the present invention bind to KRas and are capable of displacing a labeled tracer ligand occupying the KRas binding site. KRas^{WT}, KRas^{G12A}, KRas^{G12C}, KRas^{G12D}, KRas^{G12R}, KRas^{G12S}, KRas^{G12V}, KRas^{G13D}, or KRas^{Q61H} was used in the assay.

The ability of a compound to bind to KRas was measured using a TR-FRET displacement assay. Biotinylated KRas (corresponding to amino acids 1-169, produced at Accelegan Inc.) was incubated with custom made Cy5 labelled tracer, terbium streptavidin (Cisbio Inc.) and compound (1% DMSO final) in buffer (50 mM HEPES, pH 7.5, 5 mM MgCl₂, 0.005% Tween-20 and 1 mM DTT). After a 60-minute incubation at room temperature, the reaction was measured using a BMG LABTECH CLARIO star Plus via TR-FRET. 100 percent of control (POC) is determined by using a DMSO control and 0 POC is determined using a concentration of control compound that completely inhibits binding of the tracer to KRas. The POC values were fit to a 4-parameter IC₅₀ equation and the IC₅₀ values were reported (Table 1). The single point inhibition values at 10 nM were reported in Table 2.

TABLE 1
Binding to KRas (IC₅₀ nM) by Exemplary Compounds of Formula (I)

| Ex. No. | G12D | G12V | G12R | WT | G13D | Q61H | G12A | G12C | G12S |
|---|---|---|---|---|---|---|---|---|---|
| 524 | >100000 | 70875 | >100000 | | | | | | |
| 525 | 50680 | 55933 | 45416 | >100000 | >100000 | >100000 | 93754 | >100000 | >100000 |
| 526 | >100000 | >100000 | 67708 | | | | | | |
| 527 | 116 | 150 | 193 | | | | | | |
| 528 | 8 | 3 | 3 | | | | | | |
| 529 | 14 | 21 | 36 | | | | | | |
| 530 | 59 | 66 | 91 | | | | | | |
| 531 | 5 | <2 | <2 | | | | | | |
| 532 | 9 | 15 | 24 | | | | | | |
| 533 | 72 | 105 | 119 | | | | | | |
| 534 | 7 | 5 | 2 | | | | | | |
| 535 | 17 | 27 | 44 | | | | | | |
| 536 | 5 | 5 | 9 | | | | | | |
| 537 | 407 | 244 | 263 | | | | | | |
| 538 | 2 | <2 | <2 | | | | | | |
| 539 | 29 | 9 | 7 | | | | | | |
| 540 | <2 | <2 | <2 | | | | | | |
| 542 | 60 | 9 | 6 | | | | | | |
| 543 | 1689 | 152 | 354 | | | | | | |
| 544 | 22 | 3 | 4 | | | | | | |
| 545 | 4969 | 431 | 656 | | | | | | |
| 546 | 72 | 35 | 37 | | | | | | |
| 547 | 4 | 7 | 10 | | | | | | |
| 548 | 2 | 3 | 5 | | | | | | |
| 549 | 7 | 4 | 8 | | | | | | |
| 550 | 47 | 14 | 8 | | | | | | |
| 551 | 127 | 32 | 27 | | | | | | |
| 552 | 810 | 244 | 71 | | | | | | |
| 553 | 864 | 77 | 79 | | | | | | |
| 554 | 79 | 9 | 10 | | | | | | |
| 555 | 2027 | 512 | 2031 | | | | | | |
| 556 | 10 | 2 | <2 | | | | | | |
| 557 | 12 | 6 | 4 | | | | | | |
| 558 | <2 | <2 | <2 | | | | | | |
| 559 | 15 | <2 | <2 | | | | | | |
| 560 | 262 | 85 | 182 | | | | | | |
| 561 | 7 | <2 | <2 | | | | | | |
| 562 | <2 | 3 | 3 | | | | | | |
| 563 | 85 | 100 | 238 | | | | | | |
| 564 | 640 | 16 | 31 | | | | | | |
| 565 | 1155 | 908 | 1032 | | | | | | |
| 566 | 3 | 3 | <2 | | | | | | |
| 567 | 682 | 2430 | 1458 | | | | | | |
| 568 | <2 | <2 | <2 | | | | | | |
| 569 | 5 | <2 | <2 | | | | | | |
| 570 | 12 | 3 | <2 | | | | | | |
| 571 | 300 | 62 | 59 | | | | | | |
| 572 | 1966 | 266 | 162 | | | | | | |
| 574 | 7 | 5 | 7 | | | | | | |
| 575 | 25 | 7 | 27 | | | | | | |
| 576 | 45 | 10 | 37 | | | | | | |
| 577 | 29 | 606 | 727 | | | | | | |
| 578 | 5 | 8 | 2 | | | | | | |
| 579 | 5 | 6 | 3 | | | | | | |
| 580 | 296 | 81 | 30 | | | | | | |
| 581 | 160 | 39 | 41 | | | | | | |
| 582 | 3 | 11 | 10 | | | | | | |
| 583 | 33 | 14 | 3 | | | | | | |
| 584 | <2 | 6 | 12 | | | | | | |
| 585 | 434 | 103 | 76 | | | | | | |
| 586 | 6 | 3 | 4 | | | | | | |
| 587 | 323 | 64 | 74 | | | | | | |
| 588 | 1775 | 314 | 621 | | | | | | |
| 589 | 38 | 16 | 12 | | | | | | |
| 590 | 2 | 3 | 2 | | | | | | |
| 592 | 790 | 119 | 281 | | | | | | |
| 593 | 72 | 10 | 7 | | | | | | |
| 594 | 91 | 80 | 53 | | | | | | |
| 595 | 194 | 38 | 85 | | | | | | |
| 596 | 805 | 94 | 434 | | | | | | |
| 597 | 867 | 82 | 194 | | | | | | |
| 598 | 820 | 84 | 66 | | | | | | |
| 599 | 2125 | 85 | 23 | | | | | | |
| 600 | 5043 | 880 | 886 | | | | | | |
| 601 | 14 | 12 | 7 | | | | | | |
| 602 | 484 | 243 | 161 | | | | | | |
| 603 | 1627 | 590 | 453 | | | | | | |
| 604 | <2 | <2 | <2 | <2 | <2 | <2 | 2 | <2 | <2 |
| 605 | 119 | 71 | 47 | | | | | | |
| 606 | 26 | 43 | 71 | | | | | | |
| 607 | 151 | 53 | 99 | | | | | | |
| 608 | 1247 | 88 | 267 | | | | | | |
| 609 | <2 | <2 | <2 | | | | | | |
| 610 | 66 | 28 | 48 | | | | | | |
| 611 | 1529 | 121 | 127 | | | | | | |
| 612 | 4253 | 474 | 911 | | | | | | |
| 613 | 54840 | 21772 | 27461 | | | | | | |
| 614 | 805 | 398 | 701 | | | | | | |
| 615 | 1988 | 222 | 421 | | | | | | |
| 617 | 9 | 4 | 4 | | | | | | |
| 618 | 644 | 168 | 195 | | | | | | |
| 619 | 33 | 21 | 4 | | | | | | |
| 620 | 495 | 248 | 376 | | | | | | |
| 621 | 891 | 1290 | 3492 | | | | | | |
| 622 | 377 | 68 | 139 | | | | | | |
| 624 | 19 | 17 | 41 | | | | | | |
| 625 | 21 | 10 | 17 | | | | | | |
| 626 | 98785 | 42955 | 23588 | | | | | | |
| 627 | 35 | 12 | 27 | | | | | | |
| 628 | 78 | 107 | 180 | | | | | | |
| 629 | 65 | 15 | 37 | | | | | | |
| 630 | 796 | 93 | 34 | | | | | | |
| 631 | 9 | 12 | 33 | | | | | | |
| 632 | 2185 | 1196 | 1872 | | | | | | |
| 633 | 8846 | 2817 | 6611 | | | | | | |
| 634 | 2238 | 262 | 839 | | | | | | |
| 635 | 1643 | 454 | 766 | | | | | | |
| 636 | 10 | 3 | <2 | | | | | | |
| 637 | 1218 | 3788 | 2246 | | | | | | |
| 638 | 41 | 34 | 16 | | | | | | |
| 639 | 13 | 2 | 2 | | | | | | |
| 640 | 83 | 16 | 17 | | | | | | |
| 641 | 3117 | 632 | 540 | | | | | | |
| 642 | 864 | 235 | 195 | | | | | | |
| 643 | <2 | <2 | <2 | | | | | | |
| 644 | 3 | <2 | <2 | | | | | | |
| 645 | 313 | 139 | 257 | | | | | | |
| 647 | 1988 | 581 | 582 | | | | | | |
| 648 | 9120 | 6083 | 7370 | | | | | | |
| 649 | 85 | 9 | 38 | | | | | | |
| 650 | 14 | <2 | 3 | | | | | | |
| 651 | 30 | 10 | 15 | | | | | | |
| 652 | 125 | 40 | 51 | | | | | | |
| 653 | 123 | 20 | 16 | | | | | | |
| 654 | 19 | 25 | 18 | | | | | | |
| 655 | 2878 | 291 | 633 | | | | | | |
| 656 | 5 | 6 | 10 | | | | | | |
| 657 | 4 | <2 | <2 | | | | | | |
| 658 | 224 | 7 | 65 | | | | | | |
| 659 | 462 | 582 | 1605 | | | | | | |
| 660 | 2 | 7 | 2 | | | | | | |
| 661 | 680 | 1743 | 699 | | | | | | |
| 662 | 3 | <2 | 3 | | | | | | |
| 663 | 32 | 10 | 26 | | | | | | |
| 664 | 7 | 6 | 2 | | | | | | |
| 665 | 1981 | 966 | 1246 | | | | | | |
| 666 | <2 | 2 | <2 | | | | | | |
| 667 | 445 | 203 | 323 | | | | | | |
| 668 | 34 | 12 | 16 | | | | | | |
| 670 | <2 | <2 | <2 | | | | | | |
| 671 | <2 | <2 | <2 | | | | | | |
| 672 | 8 | 5 | 4 | | | | | | |
| 674 | <2 | <2 | <2 | | | | | | |
| 675 | 32 | 3 | <2 | | | | | | |
| 676 | 2 | 6 | 5 | | | | | | |
| 677 | 13 | <2 | <2 | | | | | | |
| 678 | 89 | 11 | 16 | | | | | | |
| 679 | 124 | 105 | 190 | | | | | | |
| 680 | 473 | 43 | 234 | | | | | | |
| 681 | 497 | 51 | 167 | | | | | | |
| 682 | 710 | 192 | 541 | | | | | | |
| 685 | <2 | 8 | 6 | | | | | | |
| 686 | 21 | 6 | 5 | | | | | | |
| 687 | 405 | 145 | 175 | | | | | | |
| 688 | 357 | 42 | 160 | | | | | | |
| 689 | 1651 | 98 | 349 | | | | | | |
| 690 | <2 | <2 | <2 | | | | | | |
| 691 | 5 | 2 | 6 | | | | | | |
| 693 | 509 | 25 | 38 | | | | | | |
| 694 | 106 | 17 | 38 | | | | | | |
| 695 | 4 | 5 | 8 | | | | | | |
| 696 | <2 | <2 | 3 | | | | | | |
| 697 | 4 | 6 | 7 | | | | | | |
| 698 | 2 | <2 | <2 | | | | | | |
| 701 | 4 | 5 | 9 | | | | | | |
| 703 | 5 | 7 | 14 | | | | | | |
| 704 | 5 | 5 | 11 | | | | | | |
| 705 | 12 | 13 | 30 | | | | | | |
| 706 | 3 | <2 | 2 | | | | | | |
| 707 | 9 | 4 | <2 | | | | | | |
| 708 | 19888 | 42889 | 39806 | | | | | | |
| 709 | <2 | <2 | <2 | | | | | | |
| 710 | 2 | 3 | 4 | | | | | | |
| 711 | <2 | <2 | <2 | | | | | | |
| 712 | 2 | <2 | <2 | | | | | | |
| 713 | 3 | 2 | 4 | | | | | | |
| 714 | <2 | <2 | 2 | | | | | | |
| 715 | 4 | 2 | 4 | | | | | | |
| 716 | <2 | <2 | <2 | | | | | | |
| 717 | <2 | <2 | <2 | | | | | | |
| 718 | <2 | <2 | <2 | | | | | | |
| 719 | <2 | <2 | <2 | | | | | | |
| 720 | <2 | <2 | <2 | | | | | | |
| 721 | <2 | <2 | <2 | | | | | | |
| 722 | >100000 | 42887 | 30027 | | | | | | |
| 723 | >100000 | 42824 | 27518 | | | | | | |
| 724 | 1588 | 404 | 720 | 419 | 585 | 612 | 301 | 185 | 170 |
| 725 | 1614 | 410 | 441 | 965 | 1119 | 1207 | 763 | 812 | 494 |
| 726 | 58 | 131 | 344 | 145 | 138 | 177 | 242 | 208 | 68 |
| 727 | 634 | 333 | 520 | 657 | 888 | 947 | 516 | 421 | 540 |
| 728 | 60 | 23 | 34 | 34 | 37 | 40 | 35 | 31 | 31 |
| 729 | 26 | 36 | 61 | 45 | 46 | 53 | 48 | 40 | 40 |
| 730 | 35 | 75 | 203 | 343 | 513 | 444 | 251 | 131 | 113 |
| 731 | 15 | 8 | 10 | 12 | 13 | 13 | 13 | 11 | 12 |
| 732 | 113 | 31 | 47 | 50 | 60 | 79 | 61 | 43 | 63 |
| 733 | 933 | 1048 | 2671 | 1373 | 1411 | 2284 | 1525 | 1522 | 1399 |
| 734 | 8112 | 11627 | 20598 | 10547 | 10475 | 11448 | 10304 | 10604 | 11558 |
| 735 | 1981 | 2586 | 3958 | 1840 | 2136 | 3526 | 2684 | 3126 | 2771 |
| 736 | 2331 | 664 | 640 | 139 | 93 | 26 | 173 | 111 | 75 |
| 737 | 13 | 5 | 7 | 13 | 12 | 14 | 10 | 10 | 8 |
| 738 | 17 | 3 | 2 | 11 | 10 | 9 | 11 | 9 | 9 |
| 739 | 3 | <2 | <2 | <2 | <2 | <2 | 2 | <2 | <2 |
| 740 | 4 | 5 | 11 | 9 | 8 | 6 | 10 | 7 | 9 |
| 741 | <2 | <2 | <2 | <2 | 3 | <2 | 2 | <2 | <2 |
| 742 | <2 | <2 | <2 | 3 | 3 | <2 | <2 | <2 | 2 |
| 743 | 8 | 3 | 20 | 9 | 9 | 7 | 8 | 6 | 8 |
| 744 | <2 | <2 | 2 | | | | | | |
| 745 | <2 | <2 | <2 | 4 | 3 | <2 | <2 | 3 | 5 |
| 746 | 226 | 59 | 68 | 117 | 107 | 166 | 82 | 106 | 64 |
| 747 | 272 | 67 | 76 | 107 | 94 | 180 | 36 | 107 | 61 |
| 748 | 32 | 12 | 11 | 20 | 21 | 26 | 16 | 18 | 12 |
| 749 | 54 | 19 | 16 | 34 | 37 | 44 | 25 | 29 | 19 |
| 750 | 98 | 29 | 42 | 84 | 74 | 184 | 63 | 74 | 43 |
| 751 | 372 | 93 | 139 | 167 | 172 | 185 | 136 | 165 | 91 |
| 752 | <2 | <2 | <2 | | | | | | |
| 753 | <2 | <2 | 2 | | | | | | |
| 755 | 16 | 11 | 9 | 13 | 11 | 9 | 14 | 11 | 11 |
| 756 | 11 | 10 | 17 | 13 | 13 | 13 | 14 | 12 | 13 |
| 757 | 12 | 42 | 101 | 141 | 238 | 203 | 82 | 46 | 43 |
| 758 | 5 | <2 | <2 | <2 | <2 | <2 | <2 | <2 | <2 |
| 759 | 8 | 4 | 6 | 8 | 7 | 5 | 8 | 7 | 7 |
| 760 | 76 | 67 | 170 | 88 | 72 | 66 | 91 | 73 | 76 |
| 761 | 8 | 23 | 74 | 106 | 177 | 151 | 65 | 41 | 33 |
| 762 | 2 | <2 | <2 | <2 | <2 | <2 | <2 | <2 | <2 |
| 763 | 70 | 23 | 18 | 36 | 36 | 45 | 25 | 33 | 20 |
| 764 | 5528 | 1660 | 3115 | 3094 | 2407 | 3931 | 2412 | 2394 | 1689 |
| 765 | 268 | 83 | 132 | 156 | 146 | 164 | 139 | 144 | 100 |
| 766 | 2204 | 988 | 1880 | 980 | 1206 | 954 | 1163 | 927 | 824 |
| 767 | 17 | 5 | 3 | | | | | | |
| 768 | 3 | <2 | <2 | <2 | <2 | <2 | <2 | <2 | <2 |
| 769 | <2 | <2 | <2 | <2 | <2 | <2 | <2 | <2 | <2 |
| 770 | <2 | 3 | 7 | 15 | 25 | 21 | 12 | 6 | 6 |
| 771 | 5 | 4 | 4 | 12 | 11 | 7 | 10 | 8 | 9 |
| 772 | 7 | 5 | 12 | 4 | 4 | 3 | 5 | 7 | 5 |
| 773 | 3 | <2 | 2 | | | | | | |
| 774 | 2 | <2 | <2 | | | | | | |
| 775 | 23 | 4 | 3 | | | | | | |
| 776 | 2 | 2 | 4 | | | | | | |
| 777 | 4193 | 1054 | 1324 | | | | | | |
| 778 | 6 | <2 | 4 | | | | | | |
| 779 | 2 | <2 | 2 | <2 | <2 | <2 | <2 | <2 | <2 |
| 780 | <2 | <2 | <2 | <2 | <2 | <2 | <2 | <2 | <2 |
| 781 | 2446 | 727 | 778 | 754 | 551 | 891 | 716 | 635 | 414 |
| 782 | 4650 | 2042 | 3741 | 4215 | 2548 | 4046 | 4370 | 2782 | 2573 |
| 783 | 2 | 2 | 5 | | | | | | |
| 784 | 1322 | 1924 | 4490 | 665 | 471 | 746 | 717 | 480 | 475 |
| 785 | 1300 | 435 | 787 | | | | | | |
| 786 | 423 | 110 | 160 | | | | | | |
| 787 | 4 | 3 | 6 | | | | | | |
| 788 | 2 | <2 | 4 | | | | | | |
| 789 | <2 | <2 | <2 | | | | | | |
| 790 | 11666 | 3744 | 5647 | 10643 | 7765 | 11001 | 8015 | 9217 | 5659 |
| 791 | <2 | <2 | <2 | | | | | | |
| 792 | 2 | <2 | 2 | | | | | | |
| 793 | <2 | <2 | | <2 | | | | <2 | <2 |
| 794 | <2 | <2 | | <2 | | | | <2 | <2 |
| 795 | <2 | <2 | | <2 | | | | <2 | <2 |
| 796 | <2 | <2 | | <2 | | | | <2 | <2 |
| 797 | <2 | <2 | | <2 | | | | <2 | <2 |
| 798 | <2 | <2 | | <2 | | | | <2 | <2 |
| 799 | <2 | <2 | | <2 | | | | <2 | <2 |
| 800 | <2 | <2 | | <2 | | | | <2 | <2 |
| 801 | <2 | <2 | | <2 | | | | <2 | <2 |
| 802 | 189 | 116 | 35 | | | | | 2 | 2 |
| 803 | 14 | 9 | | 20 | | | | 11 | 14 |
| 805 | <2 | <2 | 3 | | | | | | |
| 806 | <2 | 4 | | 4 | | | | 3 | 2 |
| 807 | <2 | 3 | <2 | | | | | | |
| 808 | <2 | <2 | <2 | | | | | | |
| 809 | 3 | 4 | 5 | 5 | 3 | 5 | 5 | 3 | 4 |
| 810 | 13 | 4 | 4 | | | | | | |
| 811 | 8 | 2 | 4 | | | | | | |
| 812 | 12 | 4 | 6 | | | | | | |
| 813 | <2 | <2 | 4 | | | | | | |
| 814 | <2 | <2 | 3 | | | | | | |
| 815 | 2 | <2 | 4 | | | | | | |
| 816 | 8 | 3 | 3 | | | | | | |
| 817 | 5 | <2 | 2 | | | | | | |
| 818 | 3 | 3 | 8 | | | | | | |
| 819 | 1127 | 24 | 86 | | | | | | |
| 820 | 22878 | 51952 | 76178 | 1401 | 470 | 2126 | 634 | 591 | 1506 |
| 821 | 3890 | 5854 | 11995 | 151 | 146 | 196 | 137 | 146 | 121 |
| 822 | 2178 | 3426 | 9674 | 5417 | 5406 | 5656 | 4979 | 4076 | 4662 |
| 823 | 2223 | 922 | 1378 | 1417 | 1266 | 1390 | 1428 | 1305 | 1370 |
| 824 | 11 | 4 | 8 | | | | | | |
| 825 | 6 | <2 | 3 | | | | | | |
| 826 | <2 | <2 | <2 | | | | | | |
| 827 | <2 | <2 | 3 | | | | | | |
| 828 | 11399 | 12737 | 21440 | | | | | | |
| 829 | 939 | 195 | 460 | | | | | | |
| 830 | 5 | 5 | 12 | | | | | | |
| 831 | 42 | 36 | 38 | | | | | | |
| 832 | 17125 | 4554 | 6406 | 4589 | 3884 | 4607 | 3769 | 3204 | 2942 |
| 833 | 1083 | 3545 | 9157 | 3835 | 2823 | 3670 | 5126 | 4491 | 1861 |
| 834 | 8 | 3 | 5 | | | | | | |
| 835 | 3 | <2 | <2 | | | | | | |
| 836 | 24 | 6 | 4 | | | | | | |
| 837 | <2 | <2 | <2 | | | | | | |
| 838 | 16 | 10 | 15 | | | | | | |
| 839 | 153 | 743 | 2908 | 3784 | 4063 | 3815 | 2382 | 1629 | 1259 |
| 840 | 9 | 4 | 7 | | | | | | |
| 841 | 66871 | 34006 | 62883 | | | | | | |
| 842 | 5 | <2 | <2 | | | | | | |
| 843 | 265 | 79 | 26 | | | | | | |
| 844 | 103 | 35 | 46 | | | | | | |
| 845 | 41 | 10 | 11 | | | | | | |
| 846 | 159 | 28 | 38 | | | | | | |
| 847 | 140 | 20 | 39 | | | | | | |
| 848 | 39 | 9 | 12 | | | | | | |
| 849 | 65 | 9 | 24 | | | | | | |
| 850 | 6 | 8 | 10 | | | | | | |
| 851 | 36 | 12 | 9 | | | | | | |
| 852 | <2 | <2 | <2 | | | | | | |
| 853 | <2 | <2 | <2 | | | | | | |
| 854 | <2 | <2 | <2 | | | | | | |
| 855 | 10 | 16 | 31 | 26 | 12 | 23 | 21 | 25 | 18 |
| 856 | 1191 | 2197 | 4265 | 2823 | 2469 | 2830 | 2229 | 2060 | 2507 |
| 857 | 19 | 6 | 9 | | | | | | |
| 858 | 6 | 7 | 10 | | | | | | |
| 859 | 51 | 17 | 13 | | | | | | |
| 860 | 91 | 13 | 68 | | | | | | |
| 861 | 4 | <2 | <2 | | | | | | |
| 862 | 311 | 60 | 95 | | | | | | |
| 863 | 145 | 32 | 34 | | | | | | |
| 864 | 2133 | 946 | 1793 | | | | | | |
| 865 | 809 | 310 | 282 | | | | | | |
| 866 | 102 | 3525 | 7346 | | | | | | |
| 867 | 527 | 97 | 253 | | | | | | |
| 868 | <2 | <2 | <2 | | | | | | |
| 869 | 210 | 116 | 26 | | | | | | |
| 870 | <2 | <2 | <2 | | | | | | |
| 871 | 2665 | 4413 | 8950 | 3423 | 2715 | 4527 | 3690 | 3825 | 4437 |
| 873 | 7 | 7 | 12 | | | | | | |
| 874 | <2 | 2 | 3 | | | | | | |
| 875 | 17 | 34 | 37 | | | | | | |
| 876 | 10 | 11 | 22 | 17 | 10 | 18 | 14 | 9 | 13 |
| 877 | 3 | 3 | 5 | | | | | | |
| 878 | 3 | 3 | 6 | | | | | | |
| 879 | 2 | <2 | 3 | | | | | | |
| 880 | 5 | 6 | 12 | 11 | 7 | 12 | 9 | 7 | 8 |
| 881 | 2 | 4 | 11 | 22 | 25 | 22 | 14 | 7 | 7 |
| 882 | 2 | 3 | 8 | 13 | 15 | 15 | 9 | 5 | 5 |
| 883 | 53 | 7 | 12 | | | | | | |
| 884 | <2 | <2 | <2 | | | | | | |
| 885 | <2 | <2 | <2 | | | | | | |
| 886 | 2 | 2 | 4 | | | | | | |
| 887 | 5 | 3 | 2 | | | | | | |
| 888 | 40718 | 58272 | 62260 | 579 | 567 | 539 | 375 | 199 | 201 |
| 889 | 4 | <2 | 5 | | | | | | |
| 891 | 22 | 6 | 9 | | | | | | |
| 892 | 120 | 26 | 26 | | | | | | |
| 893 | 92 | 43 | 40 | | | | | | |
| 894 | 121 | 119 | 177 | | | | | | |
| 895 | <2 | <2 | <2 | | | | | | |
| 898 | <2 | <2 | <2 | | | | | | |
| 899 | 692 | 240 | 66 | | | | | | |
| 900 | 4 | 3 | 3 | | | | | | |
| 901 | 99 | 60 | 20 | | | | | | |
| 902 | 834 | 902 | 876 | | | | | | |
| 903 | 9826 | 15134 | 11194 | | | | | | |
| 904 | <2 | <2 | <2 | | | | | | |
| 905 | 297 | 394 | 94 | | | | | | |
| 906 | 66 | 247 | 646 | | | | | | |
| 907 | 88 | 23 | 18 | | | | | | |
| 908 | 14 | 6 | 8 | | | | | | |
| 909 | 24 | 7 | 7 | | | | | | |
| 910 | 22 | 8 | 9 | | | | | | |
| 911 | 11 | 3 | 10 | | | | | | |
| 912 | 45 | 69 | 85 | | | | | | |
| 914 | 12 | 3 | 5 | | | | | | |
| 915 | <2 | <2 | <2 | | | | | | |
| 916 | 78 | 120 | 132 | | | | | | |
| 917 | 4 | 7 | 12 | | | | | | |
| 918 | <2 | <2 | | <2 | | | | <2 | <2 |
| 919 | <2 | <2 | | <2 | | | | <2 | <2 |
| 920 | 4 | <2 | | 2 | | | | <2 | <2 |
| 921 | 4 | 2 | | 4 | | | | 2 | 2 |
| 922 | 74 | 13 | | 21 | | | | 18 | 9 |
| 923 | 18 | 4 | | 8 | | | | 7 | 5 |
| 924 | 14 | 4 | | 7 | | | | 7 | 5 |
| 925 | 6 | 2 | | 3 | | | | 4 | 3 |
| 926 | 2 | <2 | | <2 | | | | <2 | <2 |
| 927 | 13 | 4 | | 6 | | | | 7 | 5 |
| 928 | 2 | <2 | | <2 | | | | <2 | <2 |
| 929 | 2 | <2 | | <2 | | | | <2 | <2 |
| 930 | <2 | <2 | | <2 | | | | <2 | <2 |
| 931 | <2 | <2 | | <2 | | | | <2 | <2 |
| 932 | <2 | <2 | | <2 | | | | <2 | <2 |
| 933 | <2 | <2 | | 4 | | | | <2 | <2 |
| 934 | <2 | <2 | | <2 | | | | <2 | <2 |
| 935 | 4 | 4 | | 7 | | | | 7 | 5 |
| 936 | <2 | <2 | | 3 | | | | 2 | <2 |
| 937 | <2 | <2 | | <2 | | | | <2 | <2 |
| 938 | <2 | <2 | | <2 | | | | <2 | <2 |
| 939 | <2 | <2 | | <2 | | | | <2 | <2 |
| 940 | <2 | <2 | | <2 | | | | <2 | <2 |
| 941 | <2 | <2 | | <2 | | | | <2 | <2 |
| 943 | <2 | <2 | | <2 | | | | <2 | <2 |
| 944 | <2 | <2 | | <2 | | | | <2 | <2 |
| 945 | 6 | 3 | | 3 | | | | 4 | <2 |
| 947 | <2 | <2 | | <2 | | | | <2 | <2 |

TABLE 2
Binding to KRas (% inhibition at 10 nM)
by Exemplary Compounds of Formula (I)

| Ex. No. | G12A | G13D | G12C | Q61H | G12S | WT |
|---|---|---|---|---|---|---|
| 538 | 78 | 91 | 94 | 94 | 89 | 81 |
| 540 | 98 | 98 | 98 | 98 | 99 | 98 |
| 547 | 49 | 76 | 56 | 70 | 58 | 68 |
| 548 | 79 | 86 | 84 | 79 | 79 | 79 |
| 559 | 70 | 82 | 89 | 82 | 82 | 73 |
| 562 | 59 | 69 | 76 | 67 | 81 | 62 |
| 568 | 94 | 98 | 96 | 98 | 98 | 97 |
| 586 | 91 | 93 | 93 | 90 | 93 | 91 |
| 643 | 88 | 99 | 99 | 100 | 98 | 98 |
| 670 | 98 | 98 | 99 | 97 | 100 | 98 |
| 671 | 97 | 93 | 98 | 94 | 98 | 93 |
| 674 | 93 | 92 | 98 | 95 | 97 | 91 |
| 690 | 93 | 90 | 97 | 92 | 96 | 91 |
| 698 | 77 | 84 | 91 | 88 | 86 | 82 |
| 699 | 87 | 96 | 96 | 96 | 93 | 91 |
| 700 | 83 | 96 | 97 | 96 | 93 | 91 |
| 706 | 82 | 89 | 89 | 88 | 85 | 84 |
| 707 | 76 | 85 | 84 | 85 | 85 | 76 |
| 709 | 80 | 97 | 95 | 99 | 94 | 91 |
| 710 | 74 | 86 | 88 | 88 | 83 | 80 |
| 712 | 88 | 90 | 94 | 90 | 90 | 86 |
| 713 | 74 | 67 | 81 | 69 | 76 | 66 |
| 714 | 89 | 85 | 94 | 87 | 95 | 85 |
| 716 | 83 | 98 | 97 | 98 | 95 | 93 |
| 717 | 90 | 94 | 93 | 92 | 92 | 92 |
| 718 | 94 | 95 | 96 | 96 | 94 | 94 |
| 719 | 95 | 98 | 97 | 98 | 97 | 96 |
| 721 | 90 | 93 | 92 | 92 | 90 | 89 |
| 737 | 47 | 53 | 53 | 46 | 60 | 44 |
| 752 | 96 | 98 | 99 | 99 | 97 | 97 |
| 767 | 54 | 55 | 58 | 48 | 66 | 48 |
| 773 | 86 | 94 | 95 | 95 | 92 | 90 |
| 776 | 80 | 84 | 85 | 80 | 83 | 78 |

### Example B

Inhibition of KRas Phosphorylation of ERK (HTRF) by Exemplary Compounds of Formula (I)

Cisbio HTRF Advanced PERK Assay Catalog #64AERPEH

Cells: MKN1, PSN1

### Procedure:

Day 1: Seed 6,000 cells/well-25 μl/well in 384-well white solid bottom plate; RPM1_10% FBS. Incubate overnight at 37° C./5% CO2.

Day 2: Echo transfer 25 nl of 10 mM compound 10 point dilution at 1:3 (Cf-10 uM) and incubate for 3 hour at 37° C./5% CO2.

Add 8.5 μl/well of 4× Lysis Buffer/25× Blocking reagent (do not dump media) and incubate for 30 min at room temperature on shaker.

Add conjugate mixture of 4.25 ul/well 1X-pERK-D2 and 1X-pERK-K diluted in Detection Buffer for a total of 8.5 μl/well.

Incubate for 4 hours at room temperature covered.

Read HTRF using ClarioStar

Cells: ASPC1, H727, A549, H460, HCT116, H358, H2009

Culture/Assay media: RPMI-1640+10% FBS

### Procedure:

### Cell Seeding

1. To harvest cells from flask using 0.05% Trypsin/EDTA solution. Add 10 mL of media to stop trypsinizing. Pipette the cells into a conical bottom 50 mL centrifuge tube and centrifuge 5 min×1000 rpm.

2. Re-suspend the cell pellet in media, take a cell count, and then adjust the cell density using fresh media.

3. Seed 6,000 cells into cell culture plate with 50 μL media. The

4. Incubate cell plate overnight in a 37° C., 5% CO2 incubator.

### Compound Titrations

1. Use Tecan to complete the compound addition. Compounds start from 10 uM top, 3-fold dilution, and 10 doses. The final DMSO concentration is 0.8%. Dispensed 0.2 uM Trametinib as Min control.

2. Incubate cell plate for 3 hrs in the incubator.

Detection with Cisbio pERK HTRF Kit

1. Dilute 1 volume of 4× lysis buffer with 3 volumes of deionized water. Then, add 100× the blocking reagent. Keep lysis buffer on the ice.

2. At the end of the compound treatment, flick-off the media.

3. Add 35 μL of lysis buffer per well using a Multidrop Combi. Then place on a plate agitator shaking at 300 rpm at 4° C. for 40 mins.

4. Make up the HTRF antibody buffer. For each assay plate, mix 50 μL of d2-conjugate antibody with 950 μL of detection buffer. Similarly, mix 50 μL of Cryptate antibody with 950 μL of detection buffer. Then mix the two diluted antibodies together.

5. Dispense 3.4 μL the antibody buffer to wells of an empty assay plate. Seal the plate and centrifuge plate 30 sec×1000 rpm.

6. At the end of the 4° C. lysis, centrifuge the lysate plates 3 mins×1500 rpm.

7. Use the Bravo to transfer 13.6 μL of lysate from cell culture plate to assay plate. Then incubate assay plate for 2 hrs at room temperature.

8. At the end of incubation, read plate on the Envision after centrifuging plate 30 sec×1000 rpm.

pERK IC_{50S} were reported in Table 3 and single point % inhibitions at 100 nM were reported in Table 4.

TABLE 3
Inhibition (HTRF IC₅₀ nM) of KRas -mediated Phosphorylation
of ERK by Exemplary Compounds of Formula (I)

| Ex. No. | AsPC-1 | H727 | MKN1 | HCT116 | A549 | H460 | H358 | H2009 | PSN1 |
|---|---|---|---|---|---|---|---|---|---|
| 526 | >10000 | >10000 | | | | | | | |
| 527 | >10000 | >10000 | | | | | | | |
| 528 | >10000 | >10000 | | | | | | | |
| 529 | >10000 | >10000 | | | | | | | |
| 530 | 2771 | 4394 | | | | | | | |
| 531 | >10000 | >10000 | | | | | | | |
| 532 | >10000 | >10000 | | | | | | | |
| 533 | 1499 | 1967 | | | | | | | |
| 534 | 5812 | 1624 | | | | | | | |
| 535 | 1618 | 2192 | | | | | | | |
| 536 | 4551 | 3439 | | | | | | | |
| 537 | 2021 | 1178 | 1483 | | | | | | >10000 |
| 538 | 3 | 6 | 4 | 13 | 6 | 5 | 3 | | 1626 |
| 539 | 129 | 179 | | | | | | | |
| 540 | 1 | 4 | 1 | 12 | 7 | 2 | 2 | | 692 |
| 541 | 198 | 240 | | 208 | 216 | 107 | | | |
| 542 | >10000 | 2330 | | | | | | | |
| 543 | >10000 | 1608 | 5952 | | | | | | >10000 |
| 544 | 455 | 79 | 25 | | | | | | >10000 |
| 546 | 1829 | 1012 | | | | | | | |
| 547 | 49 | 263 | 16 | | | 51 | | | 7525 |
| 548 | 55 | 88 | | | | | | | |
| 549 | 328 | 208 | | | | | | | |
| 550 | 590 | 293 | 645 | 1372 | 423 | | | | >10000 |
| 551 | 2500 | 814 | 1465 | | | | | | >10000 |
| 552 | 9328 | 5299 | | | | | | | |
| 553 | 5156 | 1627 | | | | | | | |
| 554 | 2383 | 410 | | | 742 | | | | |
| 555 | >10000 | >10000 | 4020 | | | | | | >10000 |
| 556 | >10000 | 2513 | | | | | | | |
| 557 | 617 | 216 | 22 | 517 | 174 | 165 | | | >10000 |
| 558 | 312 | 603 | | | 822 | | | | |
| 559 | 105 | 20 | 5 | | | | | | 9493 |
| 560 | 3097 | 724 | | | | | | | |
| 561 | 80 | 13 | 1 | 24 | 4 | 5 | 3 | | >10000 |
| 562 | 76 | 217 | 35 | 1065 | 496 | 438 | | | >10000 |
| 563 | >10000 | >10000 | >10000 | | | | | | >10000 |
| 564 | >10000 | >10000 | | | | | | | |
| 565 | >10000 | >10000 | | | | | | | |
| 566 | 760 | 138 | | 72 | 130 | 47 | | | |
| 567 | >10000 | >10000 | | | | | | | |
| 568 | 62 | 33 | 2 | 29 | 19 | 17 | | | 1376 |
| 569 | 5112 | 444 | 69 | | | | | | >10000 |
| 570 | 1774 | 280 | 56 | | 542 | | | | >10000 |
| 571 | >10000 | >10000 | | | | | | | |
| 572 | >10000 | 4192 | | | | | | | |
| 573 | 12 | 15 | | | | | | | |
| 574 | 3420 | 1127 | 78 | | | | | | >10000 |
| 575 | >10000 | >10000 | 417 | | | | | | >10000 |
| 576 | | >10000 | | | | | | | |
| 577 | 8440 | >10000 | | | | | | | |
| 578 | 294 | 258 | 1 | | 467 | 125 | | | >10000 |
| 579 | 155 | 369 | 37 | 1776 | 357 | 481 | | | >10000 |
| 580 | >10000 | >10000 | 476 | | | | | | >10000 |
| 581 | 1367 | 1172 | 527 | | | | | | >10000 |
| 582 | 130 | 328 | | | | | | | |
| 583 | 5378 | 2498 | 312 | | | | | | >10000 |
| 584 | 231 | 981 | | | | | | | |
| 585 | 4611 | 2056 | | | | | | | |
| 586 | 94 | 85 | 25 | | 65 | | | | 3771 |
| 587 | 2072 | 3086 | 926 | | | | | | >10000 |
| 588 | >10000 | >10000 | | | | | | | |
| 589 | 1540 | 731 | | | | | | | |
| 590 | 505 | 833 | | | 494 | | | | |
| 591 | 1541 | 245 | | | 357 | 138 | | | |
| 592 | >10000 | >10000 | >10000 | | | | | | >10000 |
| 593 | 1227 | 391 | 293 | | | | | | >10000 |
| 594 | 2814 | 2294 | 2347 | | | | | | >10000 |
| 595 | 1573 | 1193 | | | | | | | |
| 596 | >10000 | 2451 | | | | | | | |
| 597 | 4805 | 2356 | | | | | | | |
| 598 | >10000 | >10000 | | | | | | | |
| 599 | >10000 | >10000 | | | | | | | |
| 600 | >10000 | >10000 | | | | | | | |
| 601 | 376 | 300 | 26 | | 344 | 133 | | | >10000 |
| 602 | 5455 | 3870 | | | | | | | |
| 603 | >10000 | >10000 | | | | | | | |
| 604 | 81 | 71 | 2 | | | | | | 425 |
| 605 | 1556 | 946 | | | | | | | |
| 606 | >10000 | >10000 | | | | | | | |
| 607 | 1738 | 1771 | | | | | | | |
| 608 | >10000 | >10000 | >10000 | | | | | | >10000 |
| 609 | 1393 | 880 | 92 | | | | | | >10000 |
| 610 | 884 | 660 | | 1010 | 1303 | 256 | 662 | | |
| 611 | 4468 | 1948 | | | | | | | |
| 612 | >10000 | >10000 | | | | | | | |
| 613 | >10000 | >10000 | | | | | | | |
| 614 | >10000 | >10000 | | | | | | | |
| 615 | 7207 | >10000 | 1529 | | | | | | >10000 |
| 616 | 3625 | 1135 | | | | | | | |
| 617 | 1553 | 763 | | | | | | | |
| 618 | >10000 | >10000 | | | | | | | |
| 619 | 3621 | 1807 | | | | | | | |
| 620 | 9681 | 6797 | | | | | | | |
| 621 | >10000 | >10000 | | | | | | | |
| 622 | >10000 | >10000 | | | | | | | |
| 623 | >10000 | >10000 | | | | | | | |
| 624 | 4030 | 3993 | 834 | | | | | | >10000 |
| 625 | 4739 | 2852 | 195 | | | | | | >10000 |
| 626 | >10000 | >10000 | | | | | | | |
| 627 | >10000 | 4555 | | | | | | | |
| 628 | 3261 | 2809 | | | | | | | |
| 629 | >10000 | >10000 | | | | | | | |
| 630 | >10000 | 4423 | | | | | | | |
| 631 | 3130 | 1599 | | | | | | | |
| 632 | >10000 | >10000 | | | | | | | |
| 633 | >10000 | >10000 | >10000 | | | | | | >10000 |
| 634 | >10000 | 6210 | 2980 | | | | | | >10000 |
| 635 | >10000 | >10000 | | | | | | | |
| 636 | 4193 | 1810 | | | | | | | |
| 637 | >10000 | >10000 | >10000 | | | | | | >10000 |
| 638 | 930 | 1268 | | | | | | | |
| 639 | 1037 | 718 | | | | | | | |
| 640 | 5131 | 889 | 208 | | | | | | >10000 |
| 641 | >10000 | >10000 | | | | | | | |
| 642 | >10000 | >10000 | 2868 | | | | | | >10000 |
| 643 | 6 | 21 | 4 | 13 | 16 | 5 | 2 | | 625 |
| 644 | 147 | 340 | 10 | 411 | 299 | 160 | | | 2616 |
| 645 | 4637 | 3720 | | | | | | | |
| 646 | 8484 | >10000 | | | | | | | |
| 647 | 9344 | >10000 | | | | | | | |
| 648 | >10000 | >10000 | | | | | | | |
| 650 | 1321 | 162 | 256 | 2411 | 847 | | | | >10000 |
| 651 | 1465 | 1791 | 546 | | | | | | >10000 |
| 652 | 2279 | 1392 | | | | | | | |
| 653 | >10000 | >10000 | | | | | | | |
| 654 | 1413 | 1792 | | | | | | | |
| 655 | >10000 | >10000 | | | | | | | |
| 656 | 1896 | 2378 | 943 | | | | | | 3243 |
| 657 | | 2660 | | | | | | | |
| 659 | >10000 | >10000 | | | | | | | |
| 660 | 8033 | >10000 | | | | | | | |
| 661 | >10000 | >10000 | | | | | | | |
| 662 | 837 | 274 | 57 | | | | | | >10000 |
| 663 | 3654 | 936 | | | | | | | |
| 664 | 567 | 351 | 5 | | 152 | | | | >10000 |
| 665 | 1242 | 3125 | | | | | | | |
| 666 | 184 | 320 | 18 | | 207 | | | | >10000 |
| 667 | 6148 | 6053 | | | | | | | |
| 668 | >10000 | >10000 | | | | | | | |
| 669 | >10000 | 4511 | | | | | | | |
| 670 | 2 | 5 | 2 | 53 | 9 | 17 | 1 | | 4685 |
| 671 | 28 | 6 | 7 | | | | | | 4108 |
| 672 | 469 | 1262 | | | 541 | | | | |
| 673 | 115 | 21 | | | | | | | |
| 674 | 9 | 21 | 6 | 191 | 30 | 69 | 2 | | >10000 |
| 675 | 9493 | 2039 | | | | | | | |
| 676 | 996 | 5133 | | | | | | | |
| 677 | | 4584 | | | | | | | |
| 678 | >10000 | >10000 | | | | | | | |
| 679 | >10000 | 9493 | | | | | | | |
| 680 | >10000 | >10000 | | | | | | | |
| 681 | >10000 | >10000 | | | | | | | |
| 682 | >10000 | >10000 | | | | | | | |
| 683 | >10000 | >10000 | | | | | | | |
| 684 | >10000 | >10000 | | | | | | | |
| 685 | 245 | 1674 | 9 | 359 | 1089 | 188 | | | >10000 |
| 686 | >10000 | 8835 | | | | | | | |
| 687 | >10000 | >10000 | | | | | | | |
| 688 | >10000 | >10000 | | | | | | | |
| 689 | >10000 | >10000 | | | | | | | |
| 690 | 81 | 37 | 16 | | | | | | 7622 |
| 691 | >10000 | >10000 | | | | | | | |
| 692 | >10000 | 7028 | | | | | | | |
| 693 | >10000 | >10000 | | | | | | | |
| 694 | 985 | 605 | | | | | | | |
| 695 | 1110 | 2692 | | | | | | | |
| 696 | 690 | 948 | 123 | | | | | | >10000 |
| 697 | 1865 | 4749 | | | | | | | |
| 698 | 87 | 68 | 17 | 188 | 141 | 72 | | | 4151 |
| 699 | 2 | 6 | | 14 | 2 | 5 | 3 | | |
| 700 | 412 | 948 | 4 | 784 | 468 | 348 | 209 | | 1318 |
| 701 | 101 | 476 | | | | | | | |
| 702 | 32 | 149 | | | | | | | |
| 703 | 342 | 982 | | | | | | | |
| 704 | 53 | 223 | | | | | | | |
| 705 | 598 | 1222 | | | | | | | |
| 706 | 45 | 83 | 9 | 85 | 112 | 39 | | | 2775 |
| 707 | 79 | 24 | 2 | | | | | | 4412 |
| 708 | >10000 | >10000 | | | | | | | |
| 709 | 63 | 53 | 3 | | | | | | 253 |
| 710 | 22 | 49 | 25 | | 47 | | | | 2608 |
| 711 | 121 | 196 | 21 | 247 | | 109 | | | 6215 |
| 712 | 15 | 30 | 12 | | | 22 | | | 2708 |
| 713 | 73 | 59 | 22 | | | 102 | | | 3585 |
| 714 | 26 | 89 | 95 | 760 | | 193 | | | >10000 |
| 715 | 97 | 153 | 43 | | 344 | | | | 3623 |
| 716 | 39 | 273 | 9 | | | | | | 1019 |
| 717 | 22 | 44 | 3 | 84 | 47 | 30 | | | 1816 |
| 718 | 17 | 43 | 2 | 58 | 37 | 29 | | | 1096 |
| 719 | 7 | 23 | 1 | 23 | 7 | 8 | | | 518 |
| 720 | 19 | 47 | 6 | | | | | | 1756 |
| 721 | 15 | 28 | 2 | 48 | 19 | 12 | | | 956 |
| 725 | >10000 | >10000 | | | | | | | |
| 726 | >10000 | >10000 | | | | | | | |
| 727 | >10000 | >10000 | | | | | | | |
| 728 | 2512 | 2512 | >10000 | | | | | | >10000 |
| 729 | >10000 | >10000 | | | | | | | |
| 730 | >10000 | >10000 | | | | | | | |
| 731 | >10000 | >10000 | | | | | | | |
| 732 | >10000 | >10000 | | | | | | | |
| 737 | 420 | 79 | 9 | | | | | | 7192 |
| 738 | 5962 | 1510 | | | | | | | |
| 739 | >10000 | >10000 | | | | | | | |
| 740 | 2626 | 3160 | | | | | | | |
| 741 | >10000 | >10000 | | | | | | | |
| 742 | 1595 | 292 | 624 | 1794 | 2608 | | | | >10000 |
| 743 | 3693 | 1099 | | | | | | | |
| 744 | 2726 | 5730 | >10000 | | | | | | >10000 |
| 745 | 867 | 701 | 511 | 1712 | 815 | 516 | 185 | | 4278 |
| 746 | 2257 | 1344 | | | | | | | |
| 747 | 3848 | 1265 | | | | | | | |
| 748 | 788 | 275 | | | | | | | |
| 749 | 1405 | 246 | | | | | | | |
| 750 | 1905 | 1675 | | | | | | | |
| 751 | 5218 | 3827 | | | | | | | |
| 752 | 55 | 53 | | | | | | | |
| 753 | 69 | 83 | 14 | | | | | | 3082 |
| 754 | 747 | 516 | | | | | | | |
| 755 | >10000 | >10000 | | | | | | | |
| 756 | >10000 | >10000 | | | | | | | |
| 757 | 9835 | >10000 | | | | | | | |
| 758 | 1273 | 1784 | | | | | | | |
| 759 | >10000 | >10000 | | | | | | | |
| 760 | >10000 | >10000 | | | | | | | |
| 761 | 4321 | 1278 | >10000 | | | | | | >10000 |
| 762 | 704 | 220 | 139 | 134 | 234 | 327 | 40 | | 2550 |
| 763 | 1247 | 498 | | | | | | | |
| 764 | >10000 | >10000 | | | | | | | |
| 765 | 5910 | 2910 | | | | | | | |
| 766 | >10000 | >10000 | | | | | | | |
| 767 | 193 | 87 | 32 | 216 | 76 | 64 | | | >10000 |
| 768 | 434 | 275 | 22 | 397 | 708 | 184 | 38 | | >10000 |
| 769 | 510 | 567 | 226 | 1240 | 2188 | 661 | 88 | | >10000 |
| 770 | 444 | 654 | 5 | 3437 | 3332 | 1717 | 258 | | >10000 |
| 771 | 1370 | 897 | | | | | | | |
| 772 | 2234 | 2667 | | | | | | | |
| 773 | 7 | 8 | 4 | 18 | 8 | 10 | 3 | | 2676 |
| 774 | 852 | 138 | 123 | 506 | 924 | 434 | 121 | | 2344 |
| 775 | >10000 | >10000 | | | | | | | |
| 776 | 18 | 61 | 16 | 75 | 18 | 29 | 18 | | 9009 |
| 778 | 84 | 45 | | | | | | | |
| 779 | >10000 | 9387 | | | | | | | |
| 780 | 784 | 340 | | 557 | 412 | 246 | 33 | | |
| 781 | 8947 | >10000 | | | | | | | |
| 783 | 52 | 187 | 19 | | | | | | 9777 |
| 785 | | >10000 | | | | | | | |
| 786 | 4957 | 2821 | | | | | | | |
| 787 | 1668 | 2079 | | | | | | | |
| 788 | 73 | 211 | 13 | | | | | | 2844 |
| 789 | 32 | 116 | 6 | | | | | | 1747 |
| 791 | 1 | 2 | 0.1 | 2 | 1 | 1 | 1 | 1 | 80 |
| 792 | 4 | 32 | 2 | 13 | 9 | 4 | 8 | | 744 |
| 793 | 3 | 3 | 0.3 | 9 | 1 | 2 | 1 | | 246 |
| 794 | 32 | 27 | 1 | 26 | 12 | 6 | 5 | | 37 |
| 795 | 2 | 11 | 4 | 130 | 41 | 28 | 4 | | 4793 |
| 796 | 21 | 12 | 2 | 37 | 7 | 8 | 5 | | |
| 797 | 1 | 4 | 0.0 | 10 | 3 | 3 | 1 | | 296 |
| 798 | 17 | 19 | 0.3 | 25 | 3 | 4 | 3 | | 946 |
| 799 | 7 | 20 | 0.9 | 30 | 9 | 8 | 4 | | 988 |
| 800 | 6 | 3 | 0.3 | 13 | 3 | 3 | 1 | | 587 |
| 801 | 3 | 12 | 0.1 | 15 | 7 | 4 | 1 | | 117 |
| 802 | >10000 | >10000 | >10000 | >10000 | 12 | >10000 | 32 | | >10000 |
| 805 | 13 | 29 | 1 | 24 | 23 | 10 | 8 | | 812 |
| 806 | 2 | 364 | 19 | 129 | 139 | 107 | 43 | 29 | |
| 807 | 32 | 14 | 3 | | | | | | 125 |
| 808 | 3068 | 1687 | 653 | | | | | | >10000 |
| 809 | 871 | 809 | 57 | 1506 | 2560 | 918 | 228 | | >10000 |
| 810 | >10000 | 4982 | 110 | | | | | | >10000 |
| 811 | 149 | 68 | 37 | | | | | | 5667 |
| 812 | 743 | 363 | 68 | 324 | 922 | 166 | 177 | | >10000 |
| 813 | 1077 | 2548 | | | | | | | |
| 814 | 485 | 402 | 176 | | | | | | >10000 |
| 815 | 32 | 28 | 22 | | | | | | 2292 |
| 816 | >10000 | >10000 | | | | | | | |
| 817 | 188 | 43 | 50 | | | | | | 5106 |
| 818 | 135 | 347 | 61 | 345 | 212 | 167 | 121 | | >10000 |
| 824 | 165 | 174 | 167 | 502 | 143 | 136 | 69 | | >10000 |
| 825 | 180 | 56 | 36 | 369 | 83 | 98 | 50 | | 5916 |
| 826 | 12 | 51 | 7 | 74 | 44 | 30 | 9 | | 2394 |
| 827 | 67 | 242 | 41 | 180 | 159 | 92 | 68 | | >10000 |
| 828 | >10000 | >10000 | | | | | | | |
| 829 | >10000 | >10000 | | | | | | | |
| 830 | 272 | 267 | 188 | | | | | | 7859 |
| 831 | 331 | 1081 | 13 | | | | | | >10000 |
| 834 | 3447 | 1138 | 87 | 1204 | 2753 | 797 | 186 | | >10000 |
| 835 | 146 | 72 | 20 | 137 | 192 | 72 | 31 | | 1621 |
| 836 | 386 | 231 | 19 | 119 | 69 | 32 | 22 | | 9789 |
| 837 | 4 | 23 | 3 | 45 | 11 | 14 | 6 | | 2994 |
| 838 | 508 | 1030 | 222 | | | | | | >10000 |
| 840 | 575 | 299 | 38 | 239 | 422 | 145 | 180 | | >10000 |
| 842 | 479 | 97 | 1 | 41 | 56 | 20 | 9 | | >10000 |
| 843 | 6226 | 5295 | 55 | 305 | 2512 | 162 | 478 | | >10000 |
| 844 | 3329 | 2902 | 198 | | | | | | >10000 |
| 845 | 1566 | 1146 | 64 | 159 | 1191 | 80 | 241 | | 3462 |
| 846 | 4473 | 2852 | 102 | 572 | 2678 | 238 | 746 | | >10000 |
| 847 | 4515 | 1642 | 270 | 1427 | 1820 | 582 | 494 | | >10000 |
| 848 | 2063 | 896 | 100 | 496 | 809 | 275 | 310 | | >10000 |
| 849 | 3093 | 1000 | 189 | 1779 | 1271 | 802 | 286 | | >10000 |
| 850 | 1454 | 2282 | 4 | 171 | 862 | 83 | 150 | | >10000 |
| 851 | 1210 | 932 | | 475 | 397 | 159 | 264 | | |
| 852 | 35 | 109 | 5 | 38 | 68 | 42 | 12 | | 923 |
| 853 | 33 | 67 | 1 | 36 | 39 | 23 | 10 | | 1071 |
| 854 | 20 | 48 | 6 | 40 | 36 | 21 | 11 | | 3691 |
| 855 | >10000 | >10000 | | | | | | | |
| 857 | 967 | 352 | 6 | 410 | 611 | 144 | 369 | | >10000 |
| 858 | 138 | 375 | 3 | 21 | 19 | 7 | 43 | | >10000 |
| 859 | >10000 | 5479 | 56 | 1259 | >10000 | 892 | 1135 | | >10000 |
| 860 | 2601 | 1118 | 7 | 600 | 2763 | 395 | 455 | | >10000 |
| 861 | 1453 | 592 | 1 | 254 | 1339 | 173 | 45 | | >10000 |
| 862 | 6213 | 3704 | 196 | 2873 | 7210 | 1107 | 1683 | | >10000 |
| 863 | >10000 | 4002 | 208 | 3887 | >10000 | 1992 | 1365 | | >10000 |
| 864 | >10000 | >10000 | | | | | | | |
| 865 | 7701 | >10000 | | | | | | | |
| 867 | >10000 | 3410 | | | | | | | |
| 868 | 17 | 24 | 21 | 53 | 61 | 22 | 12 | | 3417 |
| 869 | 3597 | 4522 | 290 | 1204 | 2761 | 271 | 922 | | >10000 |
| 870 | 51 | 98 | 18 | 49 | 66 | 25 | 10 | | 1300 |
| 872 | 328 | 1206 | | 636 | 581 | 318 | 197 | | |
| 873 | 93 | 408 | 198 | 325 | 135 | 194 | 149 | | >10000 |
| 874 | 12 | 81 | 22 | 48 | 22 | 21 | 16 | | 2070 |
| 875 | >10000 | >10000 | | >10000 | >10000 | >10000 | >10000 | | |
| 876 | 661 | 1557 | 112 | 1132 | 1036 | 561 | 234 | | >10000 |
| 877 | 79 | 189 | 104 | 176 | 92 | 86 | 40 | | 2153 |
| 878 | 43 | 83 | 16 | 186 | 125 | 53 | 52 | | 4002 |
| 879 | 12 | 35 | 1 | 65 | 30 | 25 | 14 | | 889 |
| 880 | 153 | 196 | 24 | 520 | 176 | 176 | 100 | | >10000 |
| 881 | >10000 | >10000 | >10000 | | | | | | >10000 |
| 882 | 677 | 1295 | 521 | 7114 | >10000 | 4464 | 429 | | >10000 |
| 883 | 1167 | 252 | 94 | 772 | 822 | 211 | 95 | | >10000 |
| 884 | 3182 | 2807 | | | | | | | |
| 885 | 14 | 39 | 5 | 16 | 30 | 8 | 7 | | 1325 |
| 886 | 40 | 135 | 22 | 59 | 73 | 27 | 18 | | 2563 |
| 887 | >10000 | >10000 | 1241 | >10000 | >10000 | 7041 | 2535 | | >10000 |
| 889 | 600 | 129 | 55 | 188 | 365 | 134 | 24 | | >10000 |
| 890 | >10000 | >10000 | | >10000 | >10000 | >10000 | >10000 | | |
| 891 | 621 | 295 | 73 | 568 | 658 | 150 | 382 | | >10000 |
| 892 | 2177 | 1630 | 174 | 992 | 2141 | 382 | 960 | | >10000 |
| 893 | 2098 | 3019 | | | | | | | |
| 895 | 27 | 119 | 11 | 79 | 73 | 42 | 11 | | 905 |
| 896 | 6 | 2 | | | 1 | | 1 | | |
| 897 | 32 | 12 | | | 5 | | 3 | | |
| 898 | 250 | 836 | 196 | 1012 | 355 | 252 | 56 | | >10000 |
| 899 | 5367 | 4412 | | 6694 | 3534 | 1291 | 1414 | | |
| 900 | 213 | 240 | 3 | 504 | 136 | 96 | 82 | | >10000 |
| 901 | 2449 | 2416 | 19 | 1401 | 873 | 368 | 623 | | >10000 |
| 904 | 30 | 121 | 4 | 167 | 112 | 53 | 21 | | 2510 |
| 905 | 4146 | 7438 | 1142 | 6306 | 2610 | 1347 | 2705 | | >10000 |
| 907 | 2045 | 922 | 137 | 1577 | 827 | 590 | 325 | | >10000 |
| 908 | 5796 | 2306 | | 1731 | 1627 | 630 | 707 | | |
| 909 | 5161 | 1312 | | | | | | | |
| 910 | 8165 | 1670 | | | | | | | |
| 911 | 526 | 527 | 17 | 231 | 226 | 108 | 114 | | >10000 |
| 912 | 2308 | 3312 | 504 | 1829 | 2822 | 2052 | 800 | | >10000 |
| 913 | 580 | 571 | | 211 | 246 | 112 | 146 | | |
| 914 | 2382 | 1041 | | | | | 461 | | |
| 915 | 94 | 74 | 14 | 60 | 51 | 36 | 9 | | 1552 |
| 916 | >10000 | >10000 | | | | | 8968 | | |
| 917 | 149 | 223 | | 151 | 233 | 198 | 54 | | |
| 918 | 3 | 17 | 1 | 12 | 4 | 5 | 3 | | 490 |
| 919 | 14 | 43 | 3 | 37 | 19 | 10 | 14 | | 2480 |
| 920 | 77 | 42 | 4 | 44 | 19 | 15 | 12 | | 2056 |
| 921 | 92 | 86 | 0.4 | 309 | 68 | 75 | 33 | | 7628 |
| 922 | 1380 | 576 | | | | | | | |
| 923 | 365 | 125 | 9 | | | | | | >10000 |
| 924 | 262 | 118 | 14 | | | | | | >10000 |
| 925 | 826 | 302 | 9 | | | | | | >10000 |
| 926 | 66 | 29 | 0.7 | | | | | | 2999 |
| 927 | 468 | 202 | 17 | | | | | | >10000 |
| 928 | 74 | 33 | 3 | | | | | | 2841 |
| 929 | 60 | 163 | 17 | 137 | 84 | 41 | 14 | | 397 |
| 930 | 13 | 4 | 0.0 | | 3 | | 1 | | |
| 931 | 58 | 59 | 0.9 | 72 | 17 | 15 | 5 | | 2282 |
| 932 | 139 | 225 | 3 | 130 | 69 | 33 | 23 | | |
| 933 | 29 | 70 | 11 | 526 | 202 | 287 | 18 | | |
| 934 | 11 | 6 | 0.2 | | 7 | | 1 | | |
| 935 | 70 | 73 | | | | | | | |
| 936 | 17 | 25 | 2 | 262 | 71 | 104 | 11 | | |
| 937 | 116 | 116 | 1 | 88 | 55 | 36 | 12 | | |
| 938 | 9 | 4 | 0.2 | 7 | 3 | 4 | 1 | 1 | |
| 939 | 32 | 28 | 0.1 | 27 | 11 | 18 | 5 | | |
| 940 | 42 | 72 | 2 | 43 | 33 | 17 | 10 | | |
| 941 | 1 | 2 | 0.1 | 3 | 2 | 2 | 1 | 1 | |
| 942 | 2 | 1 | | 1 | 1 | 1 | 0.4 | 1 | |
| 943 | 32 | 50 | 0.4 | | 27 | | 4 | | |
| 944 | 2 | 6 | 0.0 | 6 | 6 | 3 | 1 | 1 | |
| 945 | 79 | 29 | 0.2 | 40 | 46 | 29 | 12 | | |
| 946 | 8 | 847 | | 628 | 984 | 494 | 212 | 229 | |
| 947 | 157 | 352 | 4 | | 119 | | 29 | | |

TABLE 4
Inhibition (HTRF % at 100 nM) of KRas -mediated Phosphorylation
of ERK by Exemplary Compounds of Formula (I)

| Ex. No. | AsPC-1 | H727 | H460 | H358 | A549 | HCT116 |
|---|---|---|---|---|---|---|
| 530 | | | 18 | 6 | 15 | −6 |
| 533 | | | 8 | −7 | 27 | 8 |
| 534 | | | −6 | −9 | −19 | −6 |
| 535 | | | 16 | −8 | −9 | −3 |
| 536 | | | −9 | | | 9 |
| 537 | | | 29 | | | 2 |
| 538 | | | 100 | | | 86 |
| 539 | 7 | 1 | 37 | 18 | 50 | 13 |
| 540 | 101 | 88 | 97 | 103 | 108 | 95 |
| 543 | | | −1 | | | 5 |
| 544 | −13 | 30 | 46 | 67 | 59 | 5 |
| 547 | | | | 20 | 33 | |
| 548 | | | 38 | 59 | 38 | 29 |
| 549 | | | | 3 | −6 | |
| 553 | | | 0 | 3 | 0 | 6 |
| 559 | | | 89 | 98 | 75 | 55 |
| 560 | 5 | 8 | 10 | 4 | 15 | 12 |
| 561 | 30 | 74 | 99 | 99 | 103 | 63 |
| 570 | | | 38 | 69 | 26 | 26 |
| 573 | | | 92 | 94 | 62 | 53 |
| 574 | | | 19 | | | 18 |
| 576 | 1 | −2 | −3 | | | −15 |
| 581 | | | −24 | | | 21 |
| 582 | | | 3 | 15 | 32 | 13 |
| 584 | −20 | −18 | 11 | 15 | 14 | 3 |
| 586 | | | 56 | | | 42 |
| 587 | | | 6 | | | −12 |
| 593 | | | 0 | | | −4 |
| 595 | | | 12 | −1 | 3 | −9 |
| 604 | 53 | 47 | 69 | 94 | 27 | 64 |
| 605 | | | 7 | 3 | 20 | −12 |
| 607 | | | 62 | 9 | 34 | 24 |
| 610 | | | 31 | 1 | 10 | −5 |
| 643 | | | 100 | | | 87 |
| 645 | | | 4 | | | 6 |
| 646 | −12 | −6 | −9 | 2 | −24 | −9 |
| 657 | −24 | 19 | −18 | 27 | −2 | −9 |
| 660 | | | | −20 | −41 | |
| 662 | | | | 22 | 22 | |
| 666 | | | 59 | | | 24 |
| 671 | | | 60 | 98 | 60 | 35 |
| 673 | 8 | 53 | 8 | 88 | 43 | 16 |
| 674 | | | 63 | 97 | 74 | 17 |
| 676 | | | 11 | −4 | 19 | 4 |
| 677 | −8 | 2 | | | | |
| 690 | | | 37 | 79 | 4 | 20 |
| 694 | | | −3 | 9 | −8 | −10 |
| 695 | | | −2 | −1 | 16 | 13 |
| 696 | | | 21 | | | 5 |
| 697 | | | 19 | 5 | −4 | 1 |
| 699 | | | 95 | 101 | 80 | 82 |
| 700 | | | 96 | 103 | 83 | 86 |
| 701 | | | 15 | 6 | 20 | 0 |
| 702 | 56 | 7 | 61 | | | 37 |
| 703 | | | 4 | −10 | 47 | 0 |
| 704 | 25 | 21 | 40 | 47 | 26 | 19 |
| 705 | 6 | 3 | 0 | 34 | 29 | −3 |
| 707 | | | 84 | 88 | 71 | 47 |
| 709 | | | 88 | 101 | 62 | 65 |
| 710 | | | 66 | | | 37 |
| 711 | | | | 78 | 43 | |
| 712 | | | | 95 | 52 | |
| 713 | | | | 60 | 25 | |
| 714 | | | | 73 | 16 | |
| 715 | | | 42 | | | 8 |
| 716 | | | 56 | 71 | 37 | 34 |
| 720 | 70 | 43 | 80 | 85 | 64 | 51 |
| 733 | −27 | −10 | | −2 | 7 | |
| 734 | −18 | −3 | −3 | | | −13 |
| 735 | 14 | −31 | 5 | | | −2 |
| 736 | −3 | −4 | | 5 | 2 | |
| 737 | | | 27 | 8 | 17 | 39 |
| 746 | | | 2 | −17 | −19 | 10 |
| 747 | | | −6 | −6 | 4 | 5 |
| 748 | | | −6 | 5 | −7 | 21 |
| 749 | | | −3 | −5 | 15 | 20 |
| 750 | −1 | 1 | 16 | 15 | −1 | 2 |
| 751 | −2 | −50 | | −17 | −10 | |
| 752 | 27 | 31 | 44 | 76 | 31 | 32 |
| 753 | | | 42 | 76 | 2 | 23 |
| 757 | | | | 10 | 2 | |
| 758 | | | 9 | 23 | 11 | 20 |
| 761 | | | 11 | | | −13 |
| 762 | | | 22 | | | 36 |
| 763 | | | 13 | −25 | −11 | 5 |
| 765 | −6 | −13 | | −5 | 28 | |
| 768 | | | 23 | 59 | 30 | 8 |
| 770 | | | −1 | 12 | −1 | 10 |
| 772 | | | 19 | −4 | 22 | 3 |
| 773 | | | 96 | 99 | 97 | 90 |
| 776 | | | | 84 | 60 | |
| 778 | 16 | 36 | 74 | 64 | 69 | 49 |
| 780 | −12 | 30 | −8 | 42 | 21 | −2 |
| 781 | | | | 10 | −24 | |
| 782 | 5 | −11 | | | | |
| 783 | 47 | 12 | 47 | 62 | 34 | 31 |
| 784 | 19 | 0 | 9 | | | 9 |
| 785 | −6 | 2 | 0 | 9 | 8 | 5 |
| 786 | 1 | 15 | 8 | −3 | 19 | 0 |
| 787 | 23 | 33 | 11 | 8 | 14 | 9 |
| 788 | 45 | 41 | 47 | 75 | 38 | 33 |
| 789 | 77 | 43 | 76 | 97 | 57 | 55 |
| 790 | 9 | 15 | | | | |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of Formula (I): 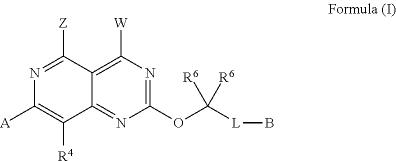
or a pharmaceutically acceptable salt thereof, wherein:
W is: 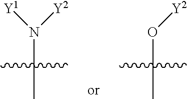
A is aryl or heteroaryl, wherein the aryl or the heteroaryl is optionally substituted with 1-4 R¹;
B is: 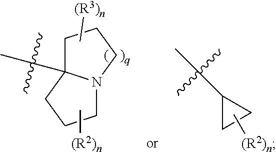
Y¹ is hydrogen, L-hydroxy optionally substituted with 1-4 R⁸, L-alkoxy optionally substituted with 1-4 R⁸, halogen, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle substituted with 1-2 oxo (═O) or oxo-containing substituent, and optionally further substituted with 1-2 R⁸;
Y² is hydrogen or C1-C4 alkyl;
or Y¹ and Y² join to form: 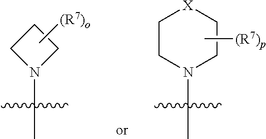
where X is selected from: a bond, —S—, —O—, —N<bound to a fused ring, —CH₂—, —CH₂—N—, —CH₂—N—CH₂—, —CH₂—CH₂—CH₂—, —CH₂—CH₂—, —O—CH₂— and —S—CH₂—;
or Y² and Z join to form V, where V is: 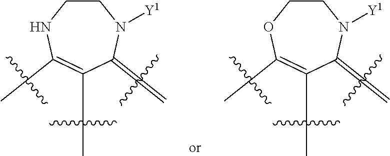
optionally substituted with 1-4 R⁸;
Z is hydrogen or joins with Y²;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
each R³ is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
wherein if V is not present at least one of R² and R³ are ═CH₂, ═CHR¹¹ or ═C(R¹¹)₂;
each R⁴ is independently hydrogen, halogen or C1-C3 alkyl;
each R⁵ is independently hydrogen or C1-C3 alkyl, or two R⁵ join to form cycloalkyl or heterocycle;
each R⁶ is independently hydrogen, hydroxy, C1-C4 hydroxyalkyl or heteroaryl,
or two R⁶ join to form C3-C6 cycloalkyl or heterocycle;
each R⁷ is independently hydrogen, C1-C3 alkyl, C2 alkenyl, hydroxy, halogen, C1-C3 haloalkyl, -L-NH₂,
—NH(C1-C3 alkyl), —N(C1-C3 alkyl)₂, oxo (═O), L-O—(C1-C3 alkyl), L-O—(C1-C3 alkyl)-OR⁵, —(C1-C3 alkyl)-OH, —C(O)OH, —C(O)O(C1-C3 alkyl), L-C(O)N(R¹⁰)₂, —NHC(O)H, —CN, aryl, —(CH₂)₁₋₂S(O)₂N(R¹⁰)₂, —NH—S(O)₂N(R¹⁰)₂, —O—S(O)₂N(R¹⁰)₂, S(O)₂R¹⁰, —P(O)(R⁵)₂ or L-heteroaryl or L-heterocycle optionally independently substituted with 1-2 substituents independently selected from C1-C3 alkyl, —CN and C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl, cyano and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a 1-2 carbon bridge;
each R⁸ is independently C1-C3 alkyl, hydroxy, halogen, —N(R¹⁰)₂, —N(R¹⁰)C(O)R¹⁰, oxo (═O), —O—(C1-C3 alkyl), —(C1-C3 alkyl)-OH, —C(O)OH, —C(O)N(R¹⁰)₂, —C(O)O(C1-C3 alkyl), —C(O)N(R¹⁰)₂, heteroaryl, heterocycle or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
each R¹⁰ is independently hydrogen, halogen, C1-C3 alkyl, C3-C4 cycloalkyl optionally substituted with 1-2 substituents independently selected from halogen and hydroxy, or two R¹⁰ join to form cycloalkyl or heterocycle optionally substituted with 1-2 C1-C3 alkyl;
each R¹¹ is independently halogen or methyl;
each L is independently a bond, —O—, —C1-C4 alkyl-, —C1-C4 alkyl-NH—, —NH—, —N(C1-C3 alkyl)- or cyclopropyl-CH₂—;
each n is 0-3;
o is 1-6;
p is 1-8; and
q is 1-2.

2. A compound of Formula (IA): 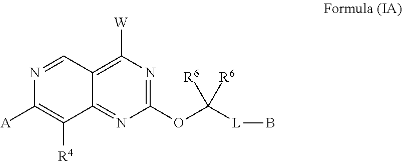
or a pharmaceutically acceptable salt thereof, wherein:
W is: 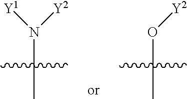
A is aryl or heteroaryl, wherein the aryl or the heteroaryl is optionally substituted with 1-4 R¹;
B is: 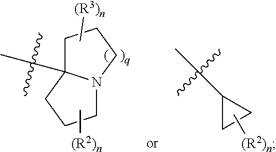
Y¹ is hydrogen, L-hydroxy optionally substituted with 1-4 R⁸, L-alkoxy optionally substituted with 1-4 R⁸, halogen, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle substituted with 1-2 oxo (═O) or oxo-containing substituent, and optionally further substituted with 1-2 R⁸;
Y² is hydrogen or C1-C4 alkyl;
or Y¹ and Y² join to form: 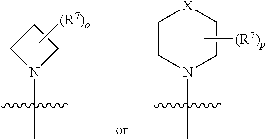
where X is selected from: a bond, —S—, —O—, —N<bound to a fused ring, —CH₂—, —CH₂—N—, —CH₂—N—CH₂—, —CH₂—CH₂—CH₂—, —CH₂—CH₂—, —O—CH₂— and —S—CH₂—;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
each R³ is independently hydrogen, hydroxy, halogen, C1-C3 haloalkyl, cyano, C1-C3 alkyl, C1-C3 cyanoalkyl, C1-C3 hydroxyalkyl, —C1-C3-N(R⁵)₂, —O—(C1-C3 alkyl), —(C1-C3 alkyl)-O—(C1-C3 alkyl), —(C1-C3 alkyl)-O—(C1-C3 alkyl)-C3-C4 cycloalkyl, HC(═O)—, -L-OC(O)N(C1-C10 alkyl)₂, —CO₂R⁵, —CO₂N(R⁵)₂, ═CH₂, ═CHR¹¹ or ═C(R¹¹)₂, or two R² join to form a heterocycle or cycloalkyl optionally substituted with C1-C3 alkyl;
each R⁴ is independently hydrogen, halogen or C1-C3 alkyl;
each R⁵ is independently hydrogen or C1-C3 alkyl, or two R⁵ join to form cycloalkyl or heterocycle;
each R⁶ is independently hydrogen, hydroxy, C1-C4 hydroxyalkyl or heteroaryl,
or two R⁶ join to form C3-C6 cycloalkyl or heterocycle;
each R⁷ is independently hydrogen, C1-C3 alkyl, C2 alkenyl, hydroxy, halogen, C1-C3 haloalkyl, -L-NH₂,
—NH(C1-C3 alkyl), —N(C1-C3 alkyl)₂, oxo (═O), L-O—(C1-C3 alkyl), L-O—(C1-C3 alkyl)-OR⁵, —(C1-C3 alkyl)-OH, —C(O)OH, —C(O)O(C1-C3 alkyl), L-C(O)N(R¹⁰)₂, —NHC(O)H—CN, aryl, —(CH₂)₁₋₂S(O)₂N(R¹⁰)₂, —NH—S(O)₂N(R¹⁰)₂, —O—S(O)₂N(R¹⁰)₂, S(O)₂R¹⁰, —P(O)(R⁵), or L-heteroaryl or L-heterocycle optionally independently substituted with 1-2 substituents independently selected from C1-C3 alkyl, —CN and C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl, cyano and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a 1-2 carbon bridge;
each R⁸ is independently C1-C3 alkyl, hydroxy, halogen, —N(R¹⁰)₂, —N(R¹⁰)C(O)R¹⁰, oxo (═O), —O—(C1-C3 alkyl), —(C1-C3 alkyl)-OH, —C(O)OH, —C(O)N(R¹⁰)₂, —C(O)O(C1-C3 alkyl), —C(O)N(R¹⁰)₂, heteroaryl, heterocycle or —CN;
each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
each R¹⁰ is independently hydrogen, halogen, C1-C3 alkyl, C3-C4 cycloalkyl optionally substituted with 1-2 substituents independently selected from halogen and hydroxy, or two R¹⁰ join to form cycloalkyl or heterocycle optionally substituted with 1-2 C1-C3 alkyl;
each R¹¹ is independently halogen or methyl;
each L is independently a bond, —O—, —C1-C4 alkyl-, —C1-C4 alkyl-NH—, —NH—, —N(C1-C3 alkyl)- or cyclopropyl-CH₂—;
each n is 0-3;
o is 1-6;
p is 1-8; and
q is 1-2.

3. The compound or salt of claim 2, wherein each R¹ is independently selected from halogen, hydroxy, C1-C3 alkoxy and C1-C4 alkyl.

4. The compound or salt of claim 2, wherein each R², if present, is selected from ═CH₂, ═CHR¹¹ or ═C(R¹¹)₂, and wherein each R³, if present, is selected from ═CH₂, ═CHR¹¹ or ═C(R¹¹)₂.

5. The compound or salt of claim 2, wherein each R⁷ is independently selected from hydrogen, C1-C4 alkyl, hydroxy, C1-C3 alkoxy, and wherein two R⁷ on non-adjacent atoms optionally join to form a 1-2 carbon bridge.

6. The compound or salt of claim 2, wherein each R⁶ is independently hydrogen or hydroxy.

7. The compound or salt of any of claims 1-6, wherein Y¹ and Y² join to form:

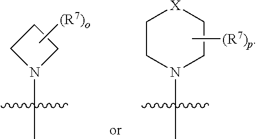

8. The compound or salt of any of claims 1-6, wherein Y¹ and Y² join to form:

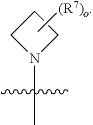

9. The compound or salt of any of claims 1-6, wherein Y¹ and Y² join to form:

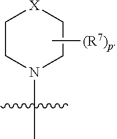

10. The compound or salt of any of claims 1-9, wherein A is naphthyl.

11. The compound or salt of any of claims 1-10, wherein at least one R¹ is C1-C4 alkyl.

12. The compound or salt of any of claims 1-10, wherein at least one R¹ is halogen

13. The compound or salt of claim 12, wherein said halogen is a fluorine.

14. The compound or salt of any of claims 1-10, wherein at least one R¹ is hydroxy.

15. The compound or salt of any of claims 1-14, wherein at least one R² is ═CH₂, ═CHR¹¹ or ═C(R¹¹)₂.

16. The compound or salt of claim 15, wherein R¹¹ is fluorine.

17. The compound or salt of any of claims 1-14, wherein at least one R² is halogen.

18. The compound or salt of claim 16, wherein said halogen is a fluorine.

19. The compound or salt of any of claims 1-14, wherein at least one R² is hydroxy.

20. The compound or salt of any of claims 1-14, wherein at least one R³ is ═CH₂, —CHR¹¹ or ═C(R¹¹)₂.

21. The compound or salt of claim 20, wherein R¹¹ is fluorine.

22. The compound or salt of any of claims 1-21, wherein at least one R³ is C1-C4 alkyl.

23. The compound or salt of any of claims 1-21, wherein at least one R³ is halogen.

24. The compound or salt of claim 23, wherein said halogen is fluorine.

25. The compound or salt of any of claims 1-21, wherein at least one R³ is hydroxy.

26. The compound or salt of any of claims 1-25, wherein R⁴ is halogen.

27. The compound or salt of claim 26, wherein said halogen is fluorine.

28. The compound or salt of any of claims 1-27, wherein at least one R⁵ is C1-C4 alkyl.

29. The compound or salt of any of claims 1-27, wherein at least one R⁵ is hydrogen.

30. The compound or salt of any of claims 1-29, wherein one or both R⁶ are C1-C4 alkyl.

31. The compound or salt of any of claims 1-29, wherein one or both R⁶ are hydrogen.

32. The compound or salt of any of claims 1-29, wherein two R⁶ join to form C3-C6 cycloalkyl or heterocycle.

33. The compound or salt of any of claims 1-6, wherein Y¹ is L-C3-C6 cycloalkyl, L-heteroaryl, L-aryl, or L-heterocycle, where L is a bond, C1-C4 alkyl, NH or N(C1-C3)alkyl.

34. The compound or salt of claim 33, wherein Y¹ is L-heteroaryl, where said heteroaryl is thietane dioxide, iso-thiazolidine dioxide, imidazopyrazine, pyridine or pyrimidine.

35. The compound or salt of claim 33, wherein Y¹ is L-C3-C6 cycloalkyl.

36. The compound or salt of claim 35, wherein the cycloalkyl is cyclobutane, cyclopentane, cyclohexane or cycloheptane.

37. The compound or salt of claim 33, wherein Y¹ is L-heterocycle.

38. The compound or salt of claim 37, wherein the heterocycle is pyrrolidinone.

39. The compound or salt of claim 1, wherein Y² is hydrogen.

40. The compound or salt of any of claims 1-6, wherein Y² is C1-C4 alkyl;

41. The compound or salt of any of claims 1-6, wherein at least one R⁸ is C1-C4 alkyl.

42. The compound or salt of any of claims 1-6, wherein at least one R⁸ is hydroxy or C1-C3 alkyl-hydroxy.

43. The compound or salt of any of claims 1-6, wherein one or two R⁸ are oxo (═O).

44. The compound or salt of any of claims 1-6, wherein at least one R⁸ is aryl or heteroaryl.

45. The compound or salt of any of claims 1-6, wherein at least one R⁸ is C(O)OH.

46. The compound or salt of any of claims 1-6, wherein at least one R⁸ is —C(O)NH₂, —C(O)NH(C1-C3 alkyl) or —C(O)N(C1-C3 alkyl)₂.

47. The compound or salt of any of claims 1-6, wherein at least one R⁸ is —NH₂, —NH(C1-C3 alkyl); —N(C1-C3 alkyl)₂.

49. The compound or salt of any of claims 1-6, wherein at least one R⁹ is C1-C4 alkyl.

50. The compound or salt of any of claims 1-6, wherein at least one R⁹ is hydroxy or C1-C3 alkyl-hydroxy.

51. The compound or salt of any of claims 1-6, wherein one or two R⁹ is oxo (═O).

52. The compound or salt of any of claims 1-6, wherein at least one R⁹ is aryl or heteroaryl.

53. The compound or salt of any of claims 1-6, wherein at least one R⁹ is C(O)OH.

54. The compound or salt of any of claims 1-6, wherein at least one R⁹ is —C(O)NH₂, —C(O)NH(C1-C3 alkyl) or —C(O)N(C1-C3 alkyl)₂.

55. The compound or salt of any of claims 1-6, wherein Y¹ and Y² join to form piperidine, azepane, azocane, thiazepine, diazepane, oxazepane, azetidine, pyrrolidine, piperazine bound to a fused ring via nitrogen or thiomorpholine.

56. The compound or salt of any of claims 1-6, wherein two R⁷ on the same atom join to form a spirocyclic ring selected from C3-C6 cycloalkyl and heterocycle, where said spirocyclic ring is optionally substituted with one or more substituents selected from oxo (═O), halogen, hydroxy, C1-C3 alkyl and —O—(C1-C3 alkyl).

57. The compound or salt of any of claims 1-6, wherein two R⁷ on adjacent atoms join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R⁸.

58. The compound or salt of any of claims 1-6, wherein two R⁷ on non-adjacent atoms join to form a 1-2 carbon bridge.

59. A compound selected from: 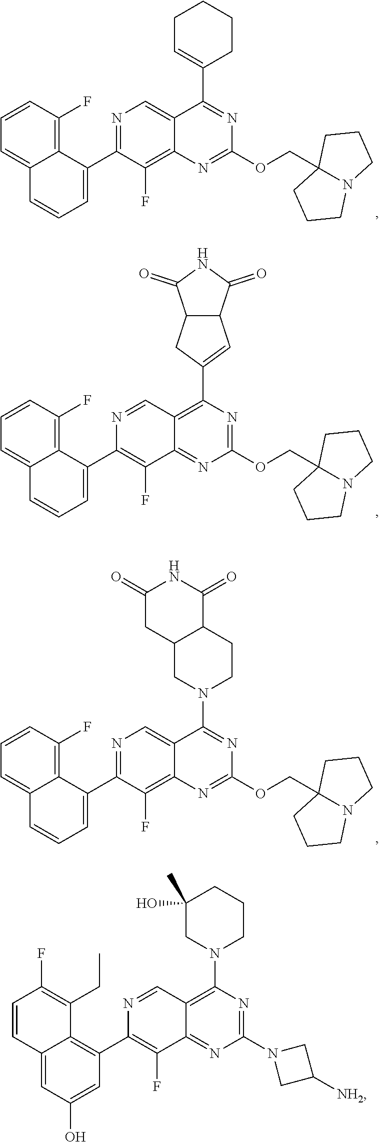 [SMILES: Fc1c(-c2cccc3cccc(F)c23)ncc2c(C3=CCCCC3)nc(OCC34CCCN3CCC4)nc12 ; O=C1NC(=O)C2CC(c3nc(OCC45CCCN4CCC5)nc4c(F)c(-c5cccc6cccc(F)c56)ncc34)=CC12 ; O=C1CC2CN(c3nc(OCC45CCCN4CCC5)nc4c(F)c(-c5cccc6cccc(F)c56)ncc34)CCC2C(=O)N1 ; CCc1c(F)ccc2cc(O)cc(-c3ncc4c(N5CCC[C@@](C)(O)C5)nc(N5CC(N)C5)nc4c3F)c12] 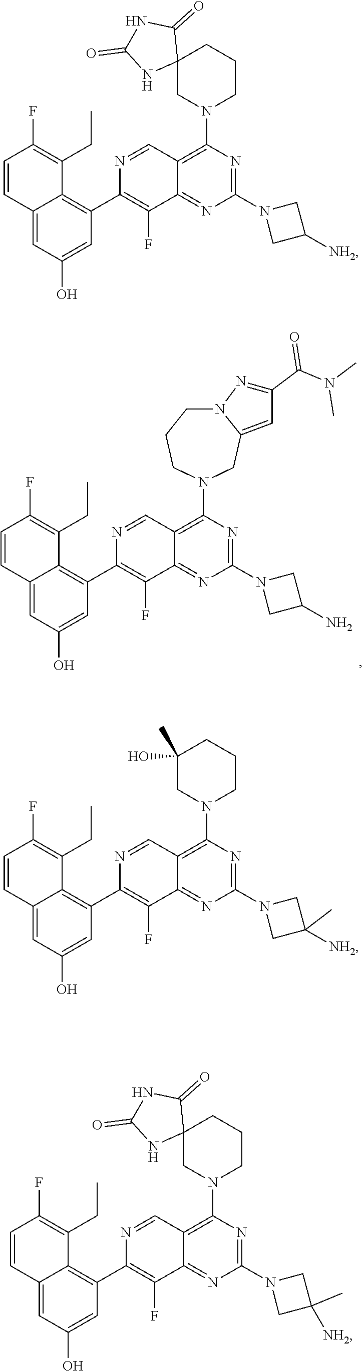 [SMILES: CCc1c(F)ccc2cc(O)cc(-c3ncc4c(N5CCCC6(C5)NC(=O)NC6=O)nc(N5CC(N)C5)nc4c3F)c12 ; CCc1c(F)ccc2cc(O)cc(-c3ncc4c(N5CCCn6nc(C(=O)N(C)C)cc6C5)nc(N5CC(N)C5)nc4c3F)c12 ; CCc1c(F)ccc2cc(O)cc(-c3ncc4c(N5CCC[C@@](C)(O)C5)nc(N5CC(C)(N)C5)nc4c3F)c12 ; CCc1c(F)ccc2cc(O)cc(-c3ncc4c(N5CCCC6(C5)NC(=O)NC6=O)nc(N5CC(C)(N)C5)nc4c3F)c12] 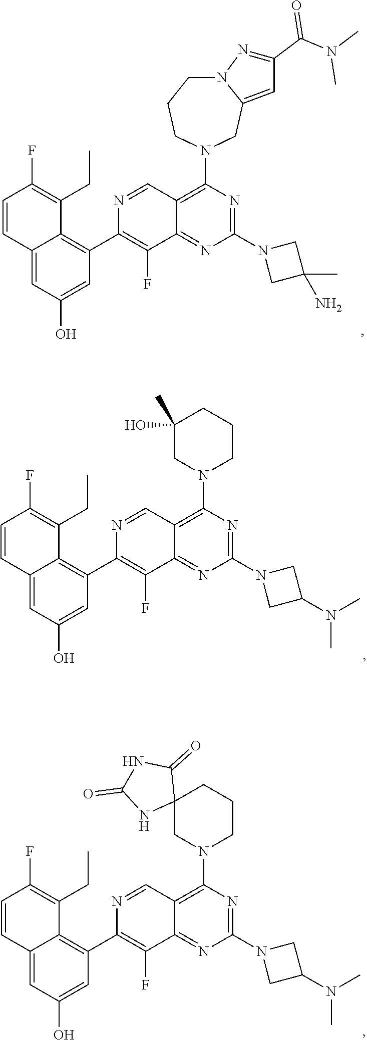 [SMILES: CCc1c(F)ccc2cc(O)cc(-c3ncc4c(N5CCCn6nc(C(=O)N(C)C)cc6C5)nc(N5CC(C)(N)C5)nc4c3F)c12 ; CCc1c(F)ccc2cc(O)cc(-c3ncc4c(N5CCC[C@@](C)(O)C5)nc(N5CC(N(C)C)C5)nc4c3F)c12 ; CCc1c(F)ccc2cc(O)cc(-c3ncc4c(N5CCCC6(C5)NC(=O)NC6=O)nc(N5CC(N(C)C)C5)nc4c3F)c12] 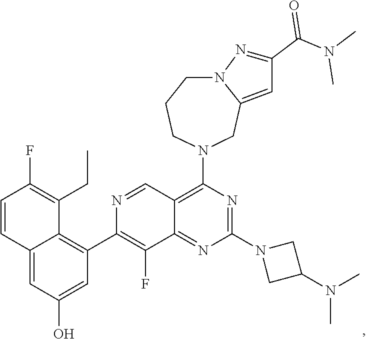 [SMILES: CCc1c(F)ccc2cc(O)cc(-c3ncc4c(N5CCCn6nc(C(=O)N(C)C)cc6C5)nc(N5CC(N(C)C)C5)nc4c3F)c12] 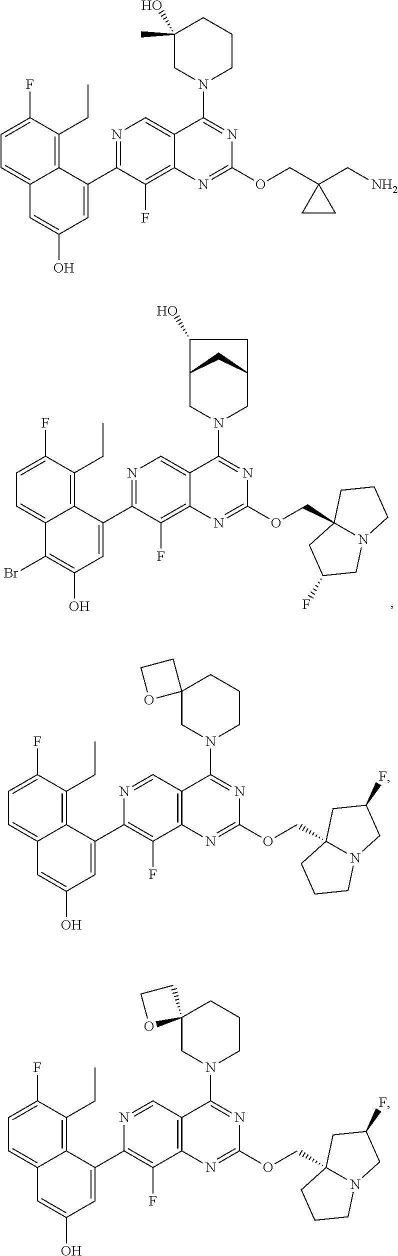 [SMILES: CCc1c(F)ccc2cc(O)cc(-c3ncc4c(N5CCC[C@@](C)(O)C5)nc(OCC5(CN)CC5)nc4c3F)c12 ; CCc1c(F)ccc2c(Br)c(O)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(O)cc(-c3ncc4c(N5CCCC6(CCO6)C5)nc(OC[C@@]56CCCN5C[C@H](F)C6)nc4c3F)c12 ; CCc1c(F)ccc2cc(O)cc(-c3ncc4c(N5CCC[C@@]6(CCO6)C5)nc(OC[C@@]56CCCN5C[C@H](F)C6)nc4c3F)c12] 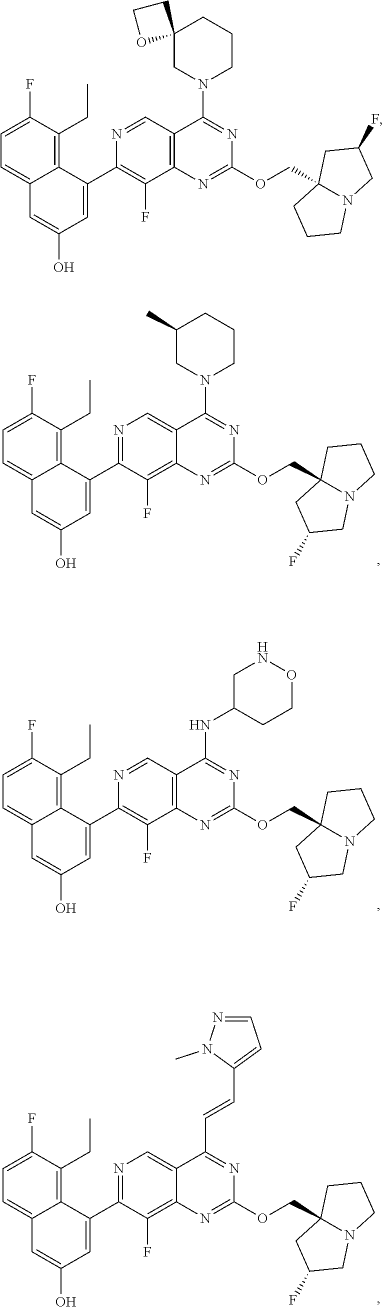 [SMILES: CCc1c(F)ccc2cc(O)cc(-c3ncc4c(N5CCC[C@]6(CCO6)C5)nc(OC[C@@]56CCCN5C[C@H](F)C6)nc4c3F)c12 ; CCc1c(F)ccc2cc(O)cc(-c3ncc4c(N5CCC[C@H](C)C5)nc(OC[C@@]56CCCN5C[C@H](F)C6)nc4c3F)c12 ; CCc1c(F)ccc2cc(O)cc(-c3ncc4c(NC5CCONC5)nc(OC[C@@]56CCCN5C[C@H](F)C6)nc4c3F)c12 ; CCc1c(F)ccc2cc(O)cc(-c3ncc4c(/C=C/c5ccnn5C)nc(OC[C@@]56CCCN5C[C@H](F)C6)nc4c3F)c12] 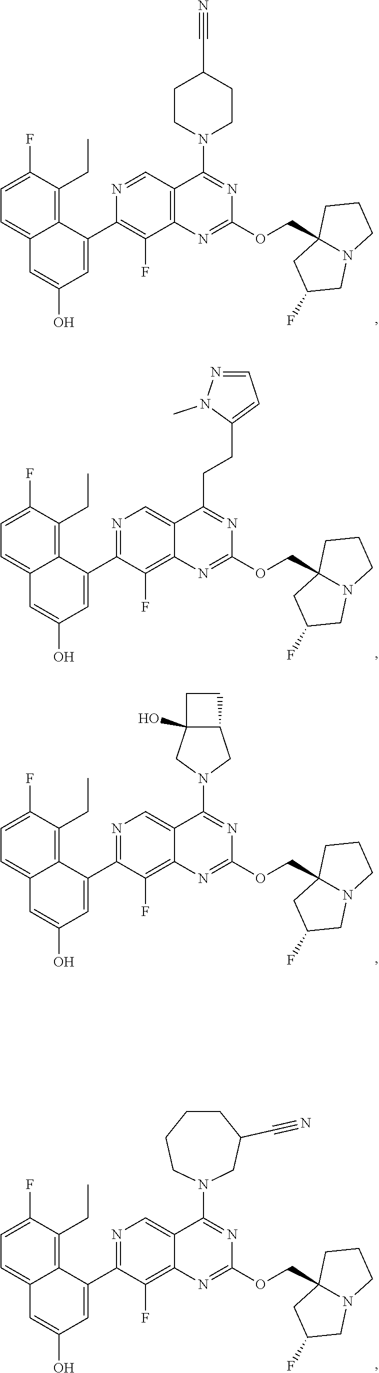 [SMILES: CCc1c(F)ccc2cc(O)cc(-c3ncc4c(N5CCC(C#N)CC5)nc(OC[C@@]56CCCN5C[C@H](F)C6)nc4c3F)c12 ; CCc1c(F)ccc2cc(O)cc(-c3ncc4c(CCc5ccnn5C)nc(OC[C@@]56CCCN5C[C@H](F)C6)nc4c3F)c12 ; CCc1c(F)ccc2cc(O)cc(-c3ncc4c(N5C[C@H]6CC[C@@]6(O)C5)nc(OC[C@@]56CCCN5C[C@H](F)C6)nc4c3F)c12 ; CCc1c(F)ccc2cc(O)cc(-c3ncc4c(N5CCCCC(C#N)C5)nc(OC[C@@]56CCCN5C[C@H](F)C6)nc4c3F)c12] 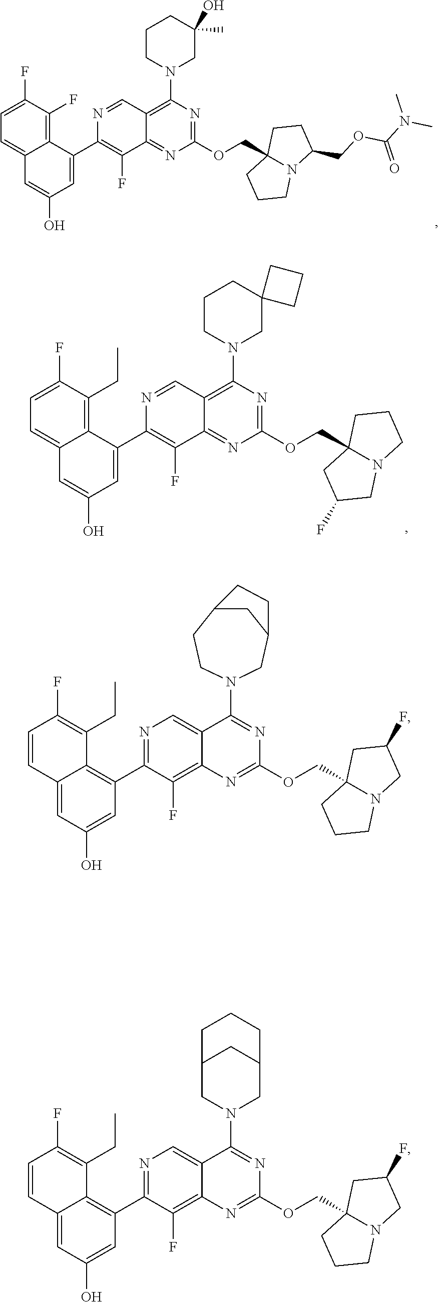 [SMILES: CN(C)C(=O)OC[C@@H]1CC[C@@]2(COc3nc(N4CCC[C@@](C)(O)C4)c4cnc(-c5cc(O)cc6ccc(F)c(F)c56)c(F)c4n3)CCCN12 ; CCc1c(F)ccc2cc(O)cc(-c3ncc4c(N5CCCC6(CCC6)C5)nc(OC[C@@]56CCCN5C[C@H](F)C6)nc4c3F)c12 ; CCc1c(F)ccc2cc(O)cc(-c3ncc4c(N5CCC6CCC(C6)C5)nc(OC[C@@]56CCCN5C[C@H](F)C6)nc4c3F)c12 ; CCc1c(F)ccc2cc(O)cc(-c3ncc4c(N5CC6CCCC(C6)C5)nc(OC[C@@]56CCCN5C[C@H](F)C6)nc4c3F)c12] 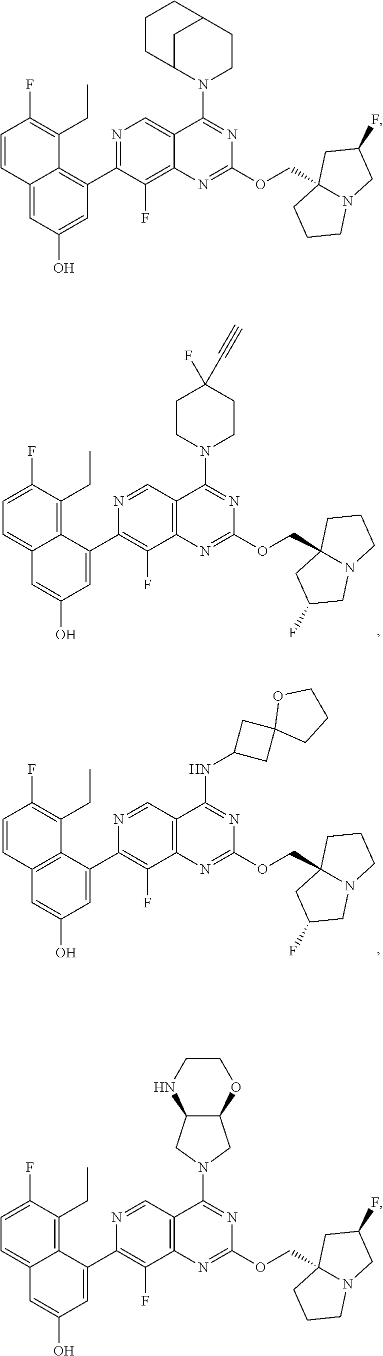 [SMILES: CCc1c(F)ccc2cc(O)cc(-c3ncc4c(N5CCC6CCCC5C6)nc(OC[C@@]56CCCN5C[C@H](F)C6)nc4c3F)c12 ; C#CC1(F)CCN(c2nc(OC[C@@]34CCCN3C[C@H](F)C4)nc3c(F)c(-c4cc(O)cc5ccc(F)c(CC)c45)ncc23)CC1 ; CCc1c(F)ccc2cc(O)cc(-c3ncc4c(NC5CC6(CCCO6)C5)nc(OC[C@@]56CCCN5C[C@H](F)C6)nc4c3F)c12 ; CCc1c(F)ccc2cc(O)cc(-c3ncc4c(N5C[C@@H]6OCCN[C@@H]6C5)nc(OC[C@@]56CCCN5C[C@H](F)C6)nc4c3F)c12] 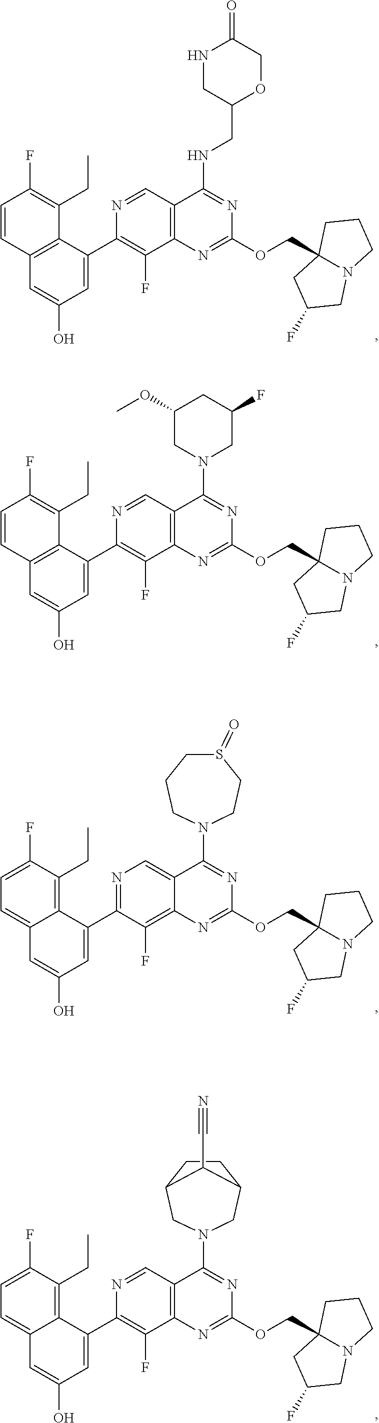 [SMILES: CCc1c(F)ccc2cc(O)cc(-c3ncc4c(NCC5CNC(=O)CO5)nc(OC[C@@]56CCCN5C[C@H](F)C6)nc4c3F)c12 ; CCc1c(F)ccc2cc(O)cc(-c3ncc4c(N5C[C@H](F)C[C@@H](OC)C5)nc(OC[C@@]56CCCN5C[C@H](F)C6)nc4c3F)c12 ; CCc1c(F)ccc2cc(O)cc(-c3ncc4c(N5CCCS(=O)CC5)nc(OC[C@@]56CCCN5C[C@H](F)C6)nc4c3F)c12 ; CCc1c(F)ccc2cc(O)cc(-c3ncc4c(N5CC6CCC(C5)C6C#N)nc(OC[C@@]56CCCN5C[C@H](F)C6)nc4c3F)c12] 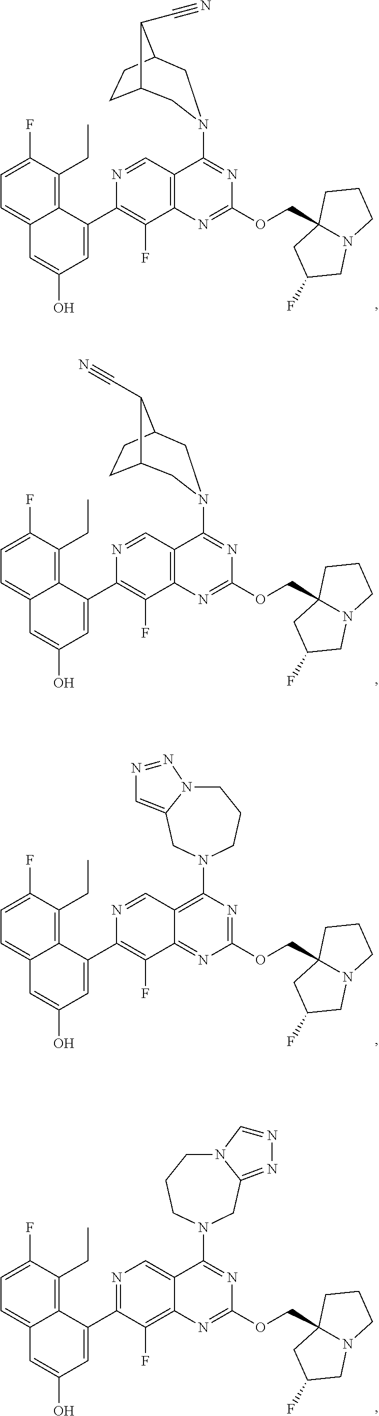 [SMILES: CCc1c(F)ccc2cc(O)cc(-c3ncc4c(N5CC6CCC(C5)C6C#N)nc(OC[C@@]56CCCN5C[C@H](F)C6)nc4c3F)c12 ; CCc1c(F)ccc2cc(O)cc(-c3ncc4c(N5CC6CCC(C5)C6C#N)nc(OC[C@@]56CCCN5C[C@H](F)C6)nc4c3F)c12 ; CCc1c(F)ccc2cc(O)cc(-c3ncc4c(N5CCCn6nncc6C5)nc(OC[C@@]56CCCN5C[C@H](F)C6)nc4c3F)c12 ; CCc1c(F)ccc2cc(O)cc(-c3ncc4c(N5CCCn6cnnc6C5)nc(OC[C@@]56CCCN5C[C@H](F)C6)nc4c3F)c12] 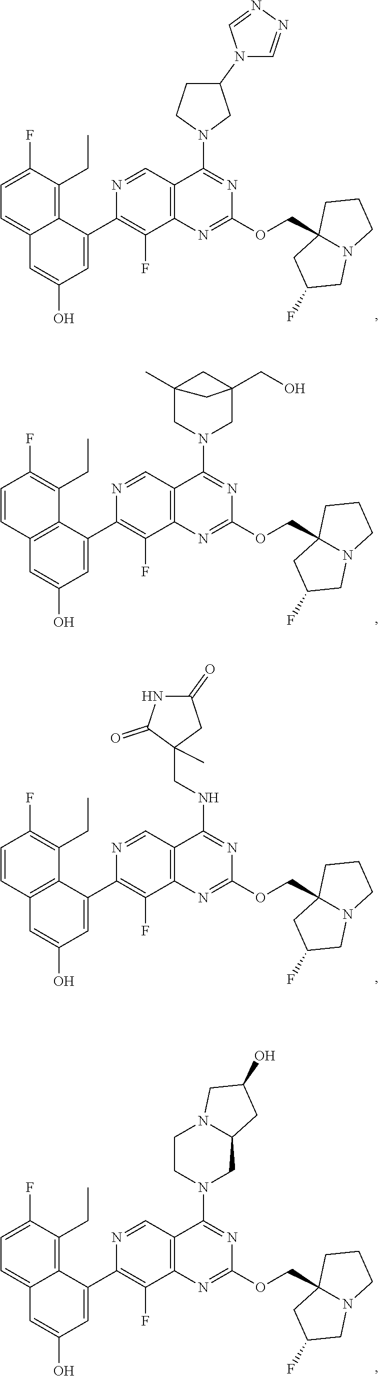 [SMILES: CCc1c(F)ccc2cc(O)cc(-c3ncc4c(N5CCC(n6cnnc6)C5)nc(OC[C@@]56CCCN5C[C@H](F)C6)nc4c3F)c12 ; CCc1c(F)ccc2cc(O)cc(-c3ncc4c(N5CC6(C)CC(CO)(C5)C6)nc(OC[C@@]56CCCN5C[C@H](F)C6)nc4c3F)c12 ; CCc1c(F)ccc2cc(O)cc(-c3ncc4c(NCC5(C)CC(=O)NC5=O)nc(OC[C@@]56CCCN5C[C@H](F)C6)nc4c3F)c12 ; CCc1c(F)ccc2cc(O)cc(-c3ncc4c(N5CCN6C[C@@H](O)C[C@H]6C5)nc(OC[C@@]56CCCN5C[C@H](F)C6)nc4c3F)c12] 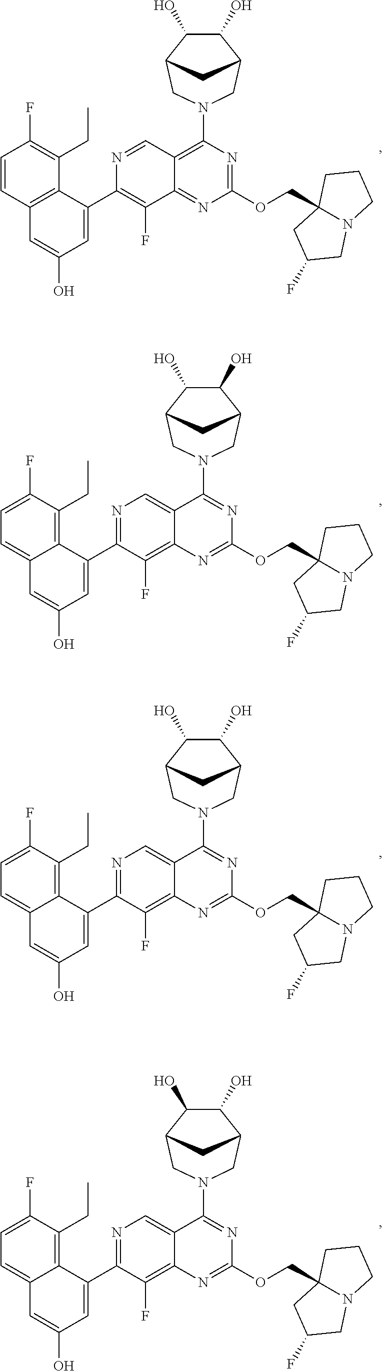 [SMILES: CCc1c(F)ccc2cc(O)cc(-c3ncc4c(N5C[C@@H]6C[C@H](C5)[C@H](O)[C@@H]6O)nc(OC[C@@]56CCCN5C[C@H](F)C6)nc4c3F)c12 ; CCc1c(F)ccc2cc(O)cc(-c3ncc4c(N5C[C@H]6C[C@@H](C5)[C@H](O)[C@H]6O)nc(OC[C@@]56CCCN5C[C@H](F)C6)nc4c3F)c12 ; CCc1c(F)ccc2cc(O)cc(-c3ncc4c(N5C[C@@H]6C[C@H](C5)[C@H](O)[C@@H]6O)nc(OC[C@@]56CCCN5C[C@H](F)C6)nc4c3F)c12 ; CCc1c(F)ccc2cc(O)cc(-c3ncc4c(N5C[C@H]6C[C@@H](C5)[C@@H](O)[C@@H]6O)nc(OC[C@@]56CCCN5C[C@H](F)C6)nc4c3F)c12] 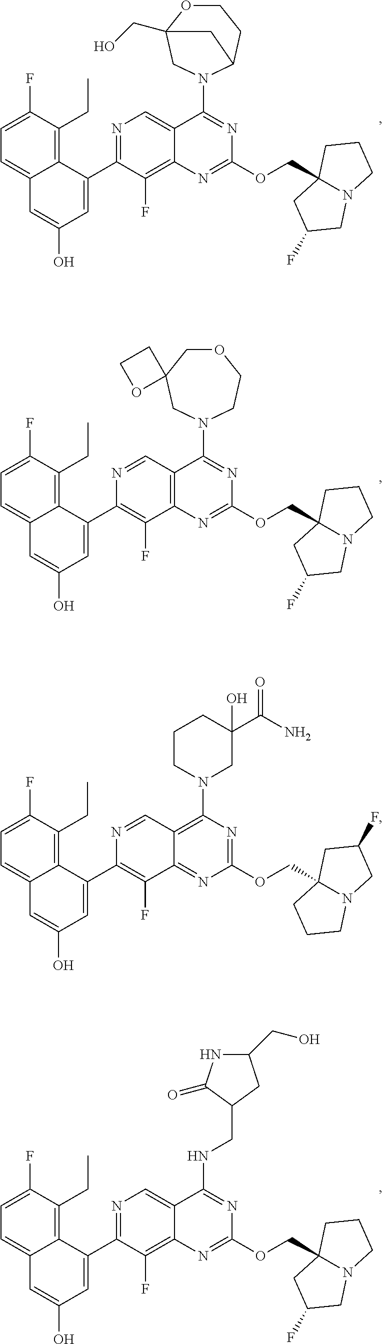 [SMILES: CCc1c(F)ccc2cc(O)cc(-c3ncc4c(N5CC6(CO)CC5CCO6)nc(OC[C@@]56CCCN5C[C@H](F)C6)nc4c3F)c12 ; CCc1c(F)ccc2cc(O)cc(-c3ncc4c(N5CCOCC6(CCO6)C5)nc(OC[C@@]56CCCN5C[C@H](F)C6)nc4c3F)c12 ; CCc1c(F)ccc2cc(O)cc(-c3ncc4c(N5CCCC(O)(C(N)=O)C5)nc(OC[C@@]56CCCN5C[C@H](F)C6)nc4c3F)c12 ; CCc1c(F)ccc2cc(O)cc(-c3ncc4c(NCC5CC(CO)NC5=O)nc(OC[C@@]56CCCN5C[C@H](F)C6)nc4c3F)c12] 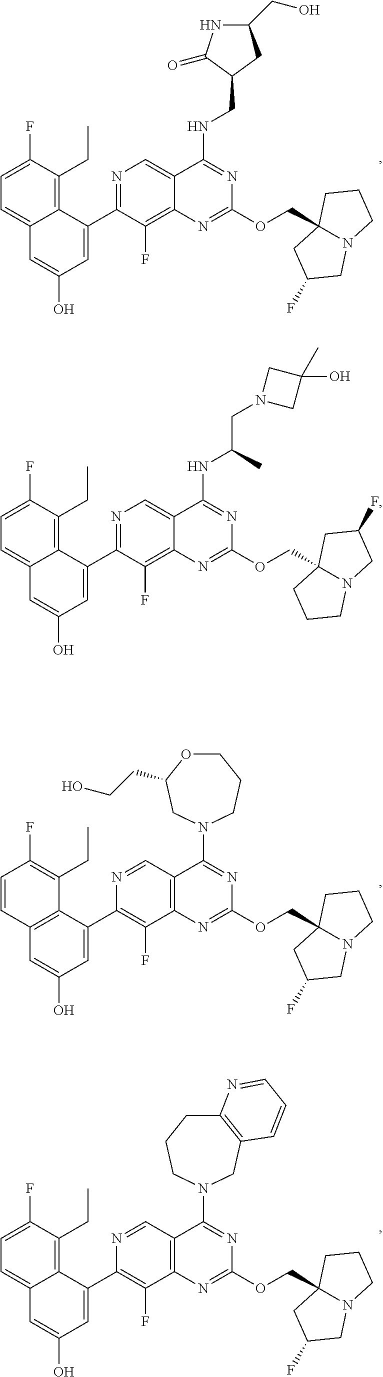 [SMILES: CCc1c(F)ccc2cc(O)cc(-c3ncc4c(NC[C@@H]5C[C@H](CO)NC5=O)nc(OC[C@@]56CCCN5C[C@H](F)C6)nc4c3F)c12 ; CCc1c(F)ccc2cc(O)cc(-c3ncc4c(N[C@H](C)CN5CC(C)(O)C5)nc(OC[C@@]56CCCN5C[C@H](F)C6)nc4c3F)c12 ; CCc1c(F)ccc2cc(O)cc(-c3ncc4c(N5CCCO[C@@H](CCO)C5)nc(OC[C@@]56CCCN5C[C@H](F)C6)nc4c3F)c12 ; CCc1c(F)ccc2cc(O)cc(-c3ncc4c(N5CCCc6ncccc6C5)nc(OC[C@@]56CCCN5C[C@H](F)C6)nc4c3F)c12] 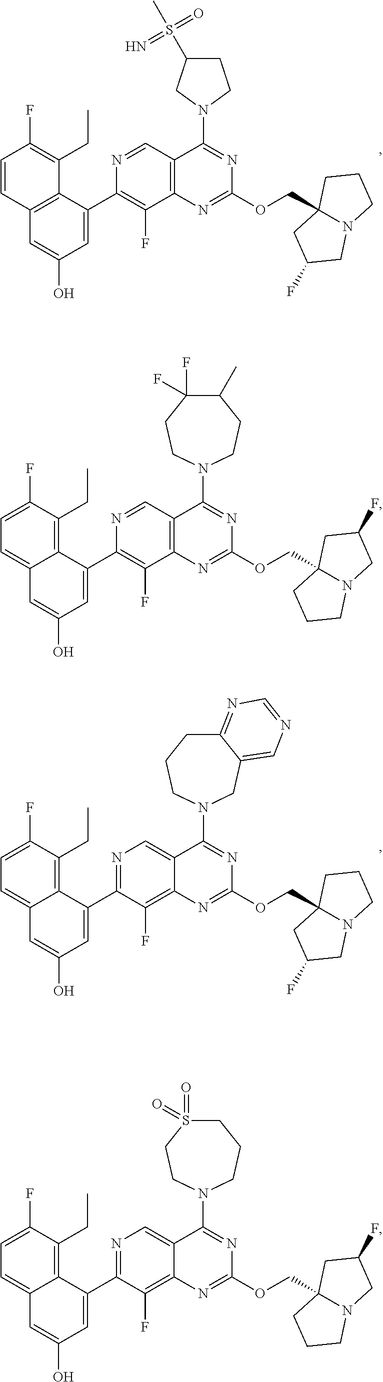 [SMILES: CCc1c(F)ccc2cc(O)cc(-c3ncc4c(N5CCC(S(C)(=N)=O)C5)nc(OC[C@@]56CCCN5C[C@H](F)C6)nc4c3F)c12 ; CCc1c(F)ccc2cc(O)cc(-c3ncc4c(N5CCC(C)C(F)(F)CC5)nc(OC[C@@]56CCCN5C[C@H](F)C6)nc4c3F)c12 ; CCc1c(F)ccc2cc(O)cc(-c3ncc4c(N5CCCc6ncncc6C5)nc(OC[C@@]56CCCN5C[C@H](F)C6)nc4c3F)c12 ; CCc1c(F)ccc2cc(O)cc(-c3ncc4c(N5CCCS(=O)(=O)CC5)nc(OC[C@@]56CCCN5C[C@H](F)C6)nc4c3F)c12] 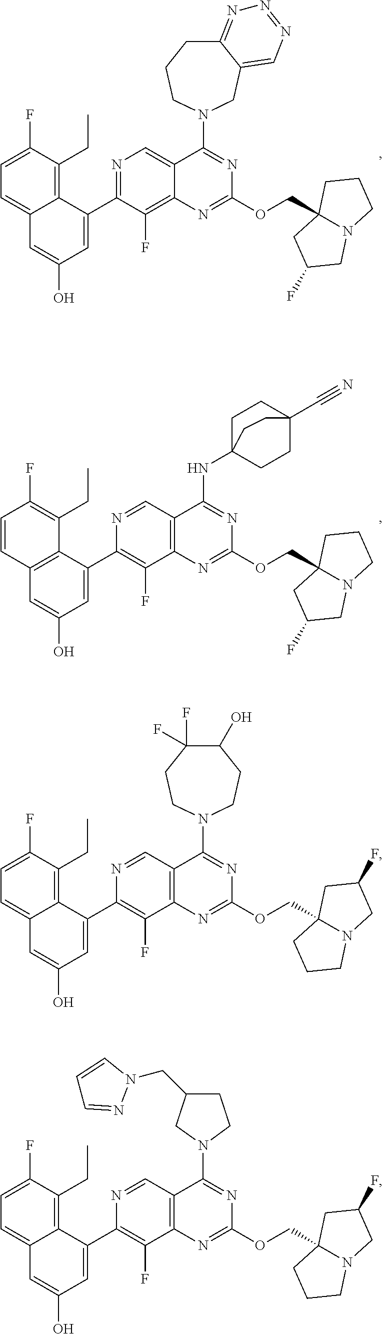 [SMILES: CCc1c(F)ccc2cc(O)cc(-c3ncc4c(N5CCCc6nnncc6C5)nc(OC[C@@]56CCCN5C[C@H](F)C6)nc4c3F)c12 ; CCc1c(F)ccc2cc(O)cc(-c3ncc4c(NC56CCC(C#N)(CC5)CC6)nc(OC[C@@]56CCCN5C[C@H](F)C6)nc4c3F)c12 ; CCc1c(F)ccc2cc(O)cc(-c3ncc4c(N5CCC(O)C(F)(F)CC5)nc(OC[C@@]56CCCN5C[C@H](F)C6)nc4c3F)c12 ; CCc1c(F)ccc2cc(O)cc(-c3ncc4c(N5CCC(Cn6cccn6)C5)nc(OC[C@@]56CCCN5C[C@H](F)C6)nc4c3F)c12] 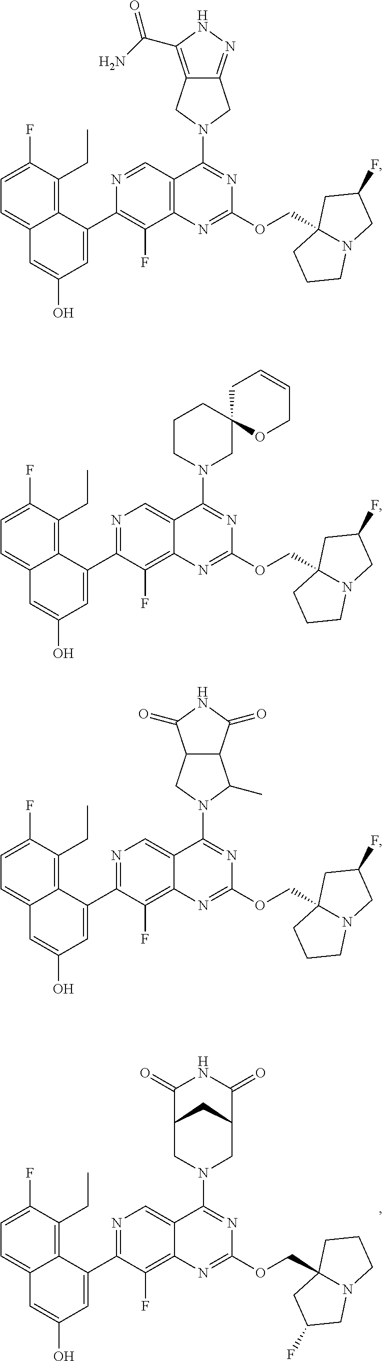 [SMILES: CCc1c(F)ccc2cc(O)cc(-c3ncc4c(N5Cc6n[nH]c(C(N)=O)c6C5)nc(OC[C@@]56CCCN5C[C@H](F)C6)nc4c3F)c12 ; CCc1c(F)ccc2cc(O)cc(-c3ncc4c(N5CCC[C@]6(CC=CCO6)C5)nc(OC[C@@]56CCCN5C[C@H](F)C6)nc4c3F)c12 ; CCc1c(F)ccc2cc(O)cc(-c3ncc4c(N5CC6C(=O)NC(=O)C6C5C)nc(OC[C@@]56CCCN5C[C@H](F)C6)nc4c3F)c12 ; CCc1c(F)ccc2cc(O)cc(-c3ncc4c(N5C[C@H]6C[C@@H](C5)C(=O)NC6=O)nc(OC[C@@]56CCCN5C[C@H](F)C6)nc4c3F)c12] 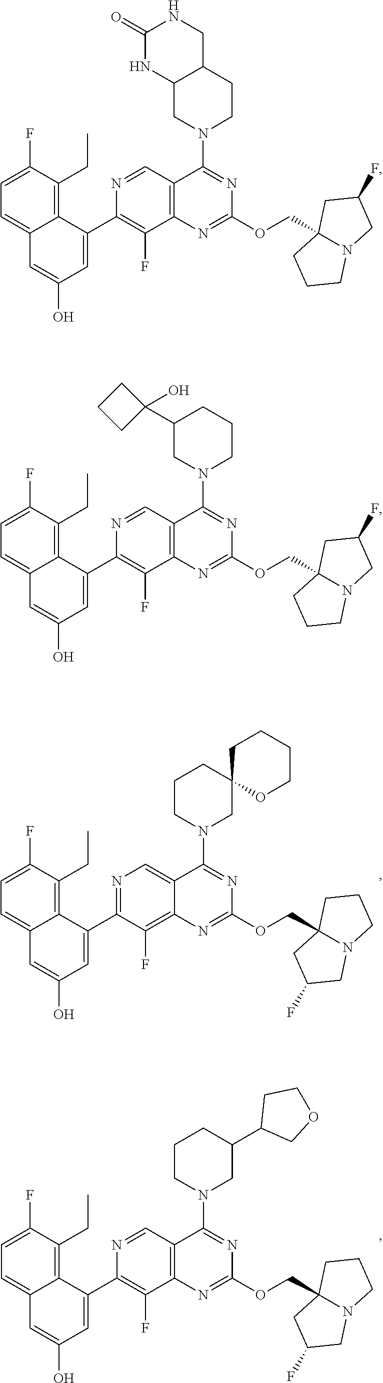 [SMILES: CCc1c(F)ccc2cc(O)cc(-c3ncc4c(N5CCC6CNC(=O)NC6C5)nc(OC[C@@]56CCCN5C[C@H](F)C6)nc4c3F)c12 ; CCc1c(F)ccc2cc(O)cc(-c3ncc4c(N5CCCC(C6(O)CCC6)C5)nc(OC[C@@]56CCCN5C[C@H](F)C6)nc4c3F)c12 ; CCc1c(F)ccc2cc(O)cc(-c3ncc4c(N5CCC[C@@]6(CCCCO6)C5)nc(OC[C@@]56CCCN5C[C@H](F)C6)nc4c3F)c12 ; CCc1c(F)ccc2cc(O)cc(-c3ncc4c(N5CCCC(C6CCOC6)C5)nc(OC[C@@]56CCCN5C[C@H](F)C6)nc4c3F)c12] 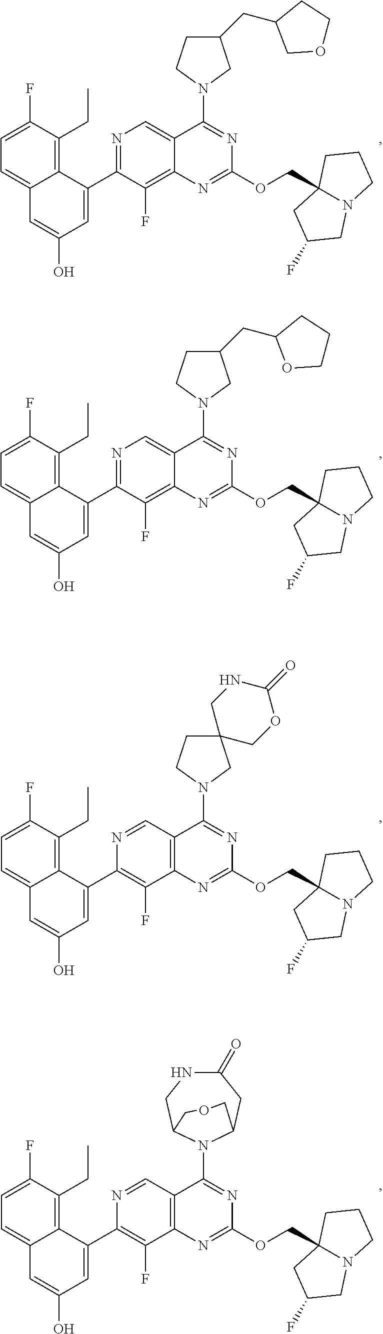 [SMILES: CCc1c(F)ccc2cc(O)cc(-c3ncc4c(N5CCC(CC6CCOC6)C5)nc(OC[C@@]56CCCN5C[C@H](F)C6)nc4c3F)c12 ; CCc1c(F)ccc2cc(O)cc(-c3ncc4c(N5CCC(CC6CCCO6)C5)nc(OC[C@@]56CCCN5C[C@H](F)C6)nc4c3F)c12 ; CCc1c(F)ccc2cc(O)cc(-c3ncc4c(N5CCC6(CNC(=O)OC6)C5)nc(OC[C@@]56CCCN5C[C@H](F)C6)nc4c3F)c12 ; CCc1c(F)ccc2cc(O)cc(-c3ncc4c(N5C6CNC(=O)CC5COC6)nc(OC[C@@]56CCCN5C[C@H](F)C6)nc4c3F)c12] 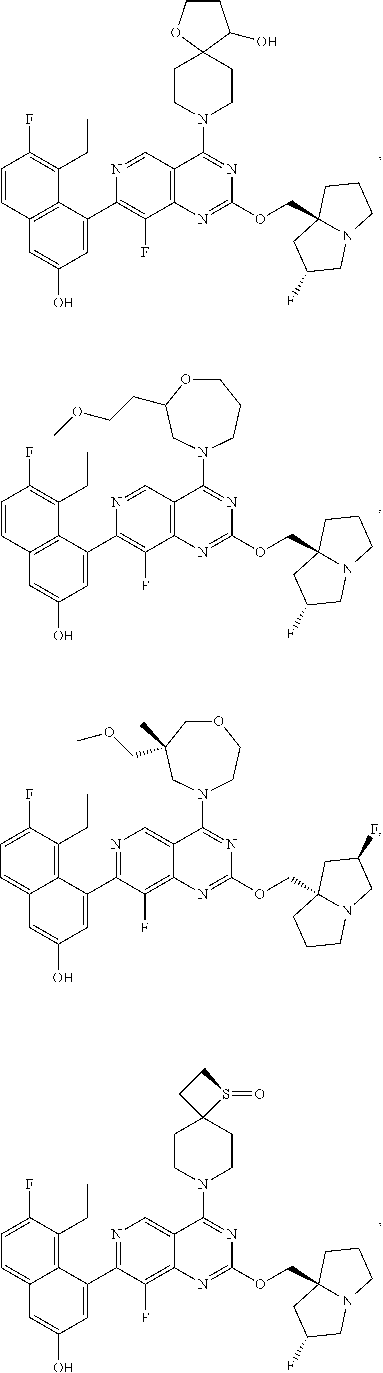 [SMILES: CCc1c(F)ccc2cc(O)cc(-c3ncc4c(N5CCC6(CC5)OCCC6O)nc(OC[C@@]56CCCN5C[C@H](F)C6)nc4c3F)c12 ; CCc1c(F)ccc2cc(O)cc(-c3ncc4c(N5CCCOC(CCOC)C5)nc(OC[C@@]56CCCN5C[C@H](F)C6)nc4c3F)c12 ; CCc1c(F)ccc2cc(O)cc(-c3ncc4c(N5CCOC[C@@](C)(COC)C5)nc(OC[C@@]56CCCN5C[C@H](F)C6)nc4c3F)c12 ; CCc1c(F)ccc2cc(O)cc(-c3ncc4c(N5CCC6(CC5)CC[S@]6=O)nc(OC[C@@]56CCCN5C[C@H](F)C6)nc4c3F)c12] 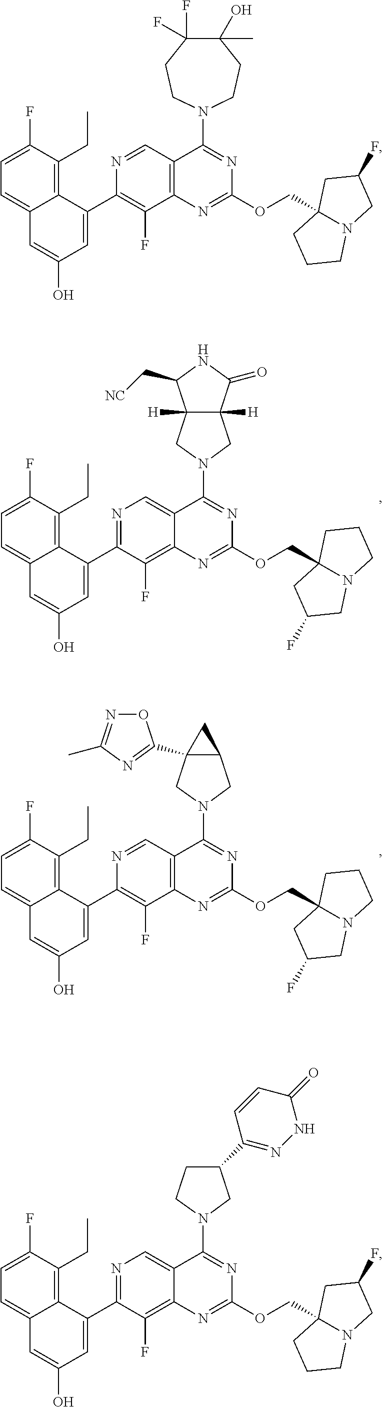 [SMILES: CCc1c(F)ccc2cc(O)cc(-c3ncc4c(N5CCC(C)(O)C(F)(F)CC5)nc(OC[C@@]56CCCN5C[C@H](F)C6)nc4c3F)c12 ; CCc1c(F)ccc2cc(O)cc(-c3ncc4c(N5C[C@@H]6[C@@H](CC#N)NC(=O)[C@@H]6C5)nc(OC[C@@]56CCCN5C[C@H](F)C6)nc4c3F)c12 ; CCc1c(F)ccc2cc(O)cc(-c3ncc4c(N5C[C@@H]6C[C@]6(c6nc(C)no6)C5)nc(OC[C@@]56CCCN5C[C@H](F)C6)nc4c3F)c12 ; CCc1c(F)ccc2cc(O)cc(-c3ncc4c(N5CC[C@H](c6ccc(=O)[nH]n6)C5)nc(OC[C@@]56CCCN5C[C@H](F)C6)nc4c3F)c12] 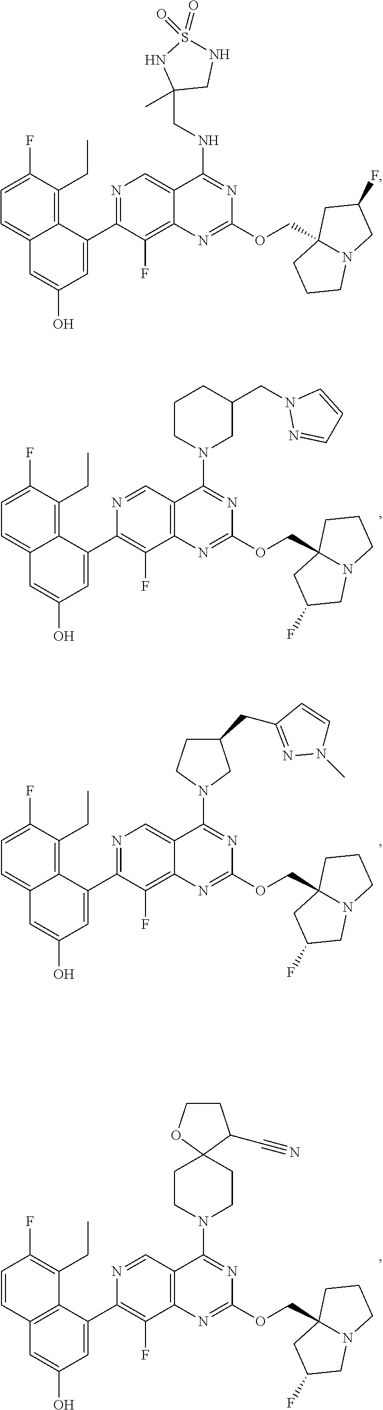 [SMILES: CCc1c(F)ccc2cc(O)cc(-c3ncc4c(NCC5(C)CNS(=O)(=O)N5)nc(OC[C@@]56CCCN5C[C@H](F)C6)nc4c3F)c12 ; CCc1c(F)ccc2cc(O)cc(-c3ncc4c(N5CCCC(Cn6cccn6)C5)nc(OC[C@@]56CCCN5C[C@H](F)C6)nc4c3F)c12 ; CCc1c(F)ccc2cc(O)cc(-c3ncc4c(N5CC[C@@H](Cc6ccn(C)n6)C5)nc(OC[C@@]56CCCN5C[C@H](F)C6)nc4c3F)c12 ; CCc1c(F)ccc2cc(O)cc(-c3ncc4c(N5CCC6(CC5)OCCC6C#N)nc(OC[C@@]56CCCN5C[C@H](F)C6)nc4c3F)c12] 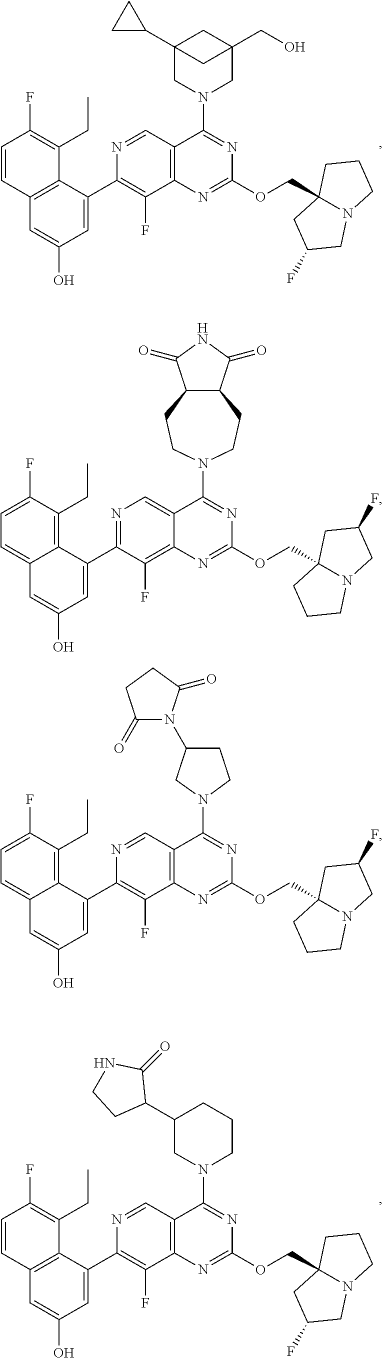 [SMILES: CCc1c(F)ccc2cc(O)cc(-c3ncc4c(N5CC6(CO)CC(C7CC7)(C5)C6)nc(OC[C@@]56CCCN5C[C@H](F)C6)nc4c3F)c12 ; CCc1c(F)ccc2cc(O)cc(-c3ncc4c(N5CC[C@@H]6C(=O)NC(=O)[C@@H]6CC5)nc(OC[C@@]56CCCN5C[C@H](F)C6)nc4c3F)c12 ; CCc1c(F)ccc2cc(O)cc(-c3ncc4c(N5CCC(N6C(=O)CCC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12] 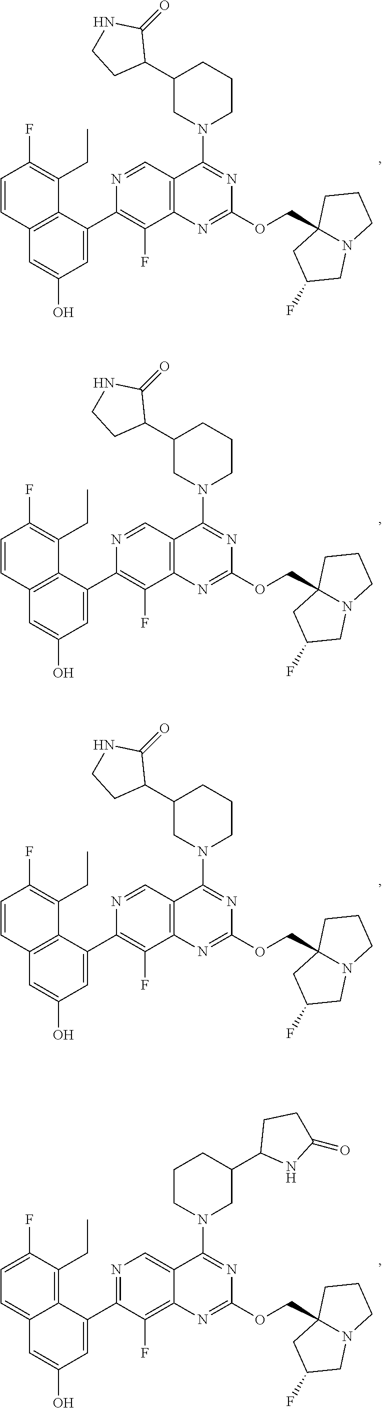 [SMILES: CCc1c(F)ccc2cc(O)cc(-c3ncc4c(N5CCCC(C6CCNC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12 ; CCc1c(F)ccc2cc(O)cc(-c3ncc4c(N5CCCC(C6CCNC6=O)C5)nc(OC[C@@]56CCCN5C[C@H](F)C6)nc4c3F)c12 ; CCc1c(F)ccc2cc(O)cc(-c3ncc4c(N5CCCC(C6CCC(=O)N6)C5)nc(OC[C@@]56CCCN5C[C@H](F)C6)nc4c3F)c12] 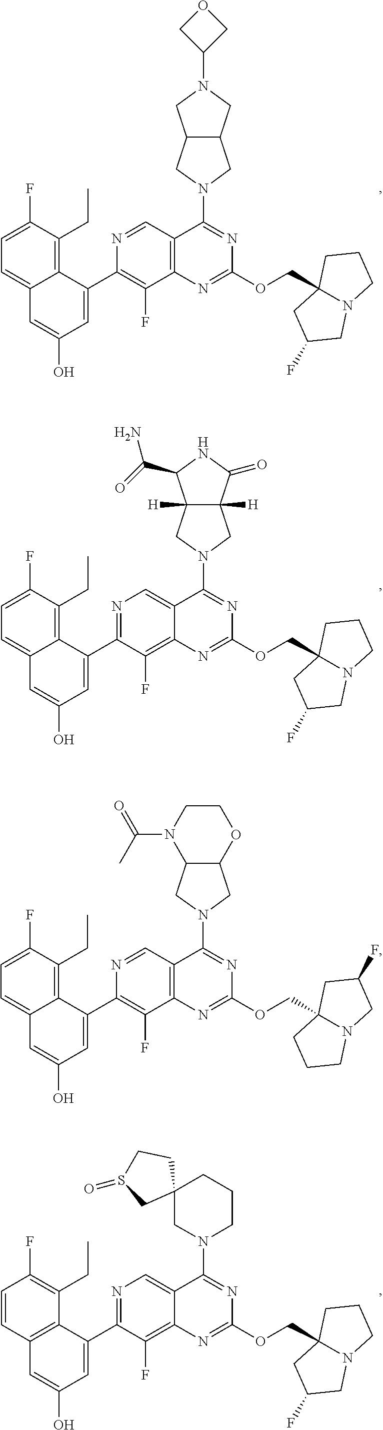 [SMILES: CCc1c(F)ccc2cc(O)cc(-c3ncc4c(N5CC6CN(C7COC7)CC6C5)nc(OC[C@@]56CCCN5C[C@H](F)C6)nc4c3F)c12 ; CCc1c(F)ccc2cc(O)cc(-c3ncc4c(N5C[C@@H]6[C@@H](C(N)=O)NC(=O)[C@@H]6C5)nc(OC[C@@]56CCCN5C[C@H](F)C6)nc4c3F)c12 ; CCc1c(F)ccc2cc(O)cc(-c3ncc4c(N5CC6OCCN(C(C)=O)C6C5)nc(OC[C@@]56CCCN5C[C@H](F)C6)nc4c3F)c12 ; CCc1c(F)ccc2cc(O)cc(-c3ncc4c(N5CCC[C@@]6(CC[S@](=O)C6)C5)nc(OC[C@@]56CCCN5C[C@H](F)C6)nc4c3F)c12] 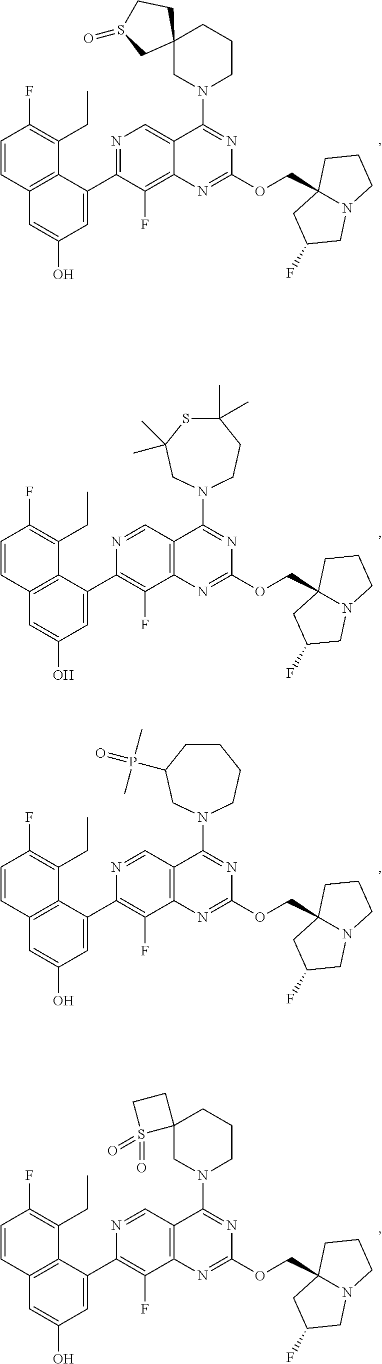 [SMILES: CCc1c(F)ccc2cc(O)cc(-c3ncc4c(N5CCC[C@]6(CC[S@](=O)C6)C5)nc(OC[C@@]56CCCN5C[C@H](F)C6)nc4c3F)c12 ; CCc1c(F)ccc2cc(O)cc(-c3ncc4c(N5CCC(C)(C)SC(C)(C)C5)nc(OC[C@@]56CCCN5C[C@H](F)C6)nc4c3F)c12 ; CCc1c(F)ccc2cc(O)cc(-c3ncc4c(N5CCCCC(P(C)(C)=O)C5)nc(OC[C@@]56CCCN5C[C@H](F)C6)nc4c3F)c12 ; CCc1c(F)ccc2cc(O)cc(-c3ncc4c(N5CCCC6(CCS6(=O)=O)C5)nc(OC[C@@]56CCCN5C[C@H](F)C6)nc4c3F)c12] 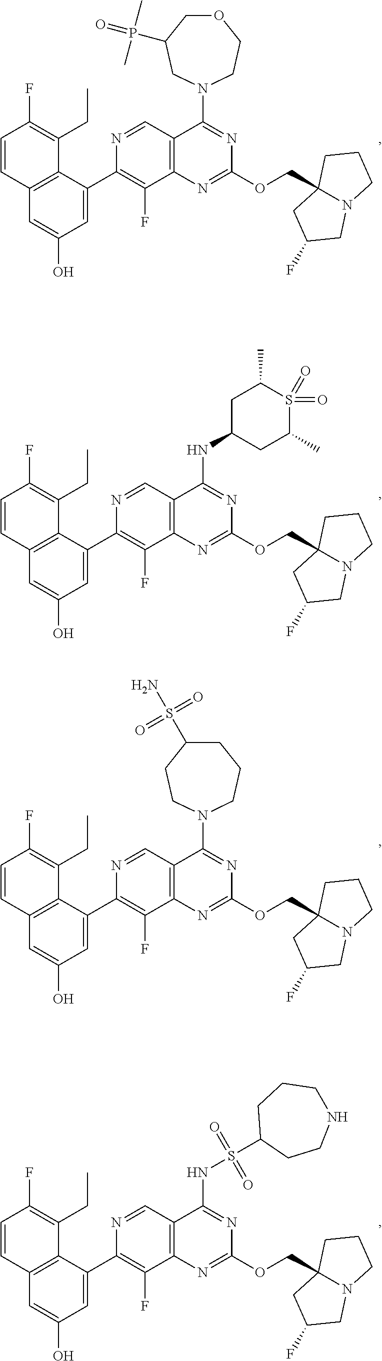 [SMILES: CCc1c(F)ccc2cc(O)cc(-c3ncc4c(N5CCOCC(P(C)(C)=O)C5)nc(OC[C@@]56CCCN5C[C@H](F)C6)nc4c3F)c12 ; CCc1c(F)ccc2cc(O)cc(-c3ncc4c(N[C@@H]5C[C@@H](C)S(=O)(=O)[C@@H](C)C5)nc(OC[C@@]56CCCN5C[C@H](F)C6)nc4c3F)c12 ; CCc1c(F)ccc2cc(O)cc(-c3ncc4c(N5CCCC(S(N)(=O)=O)CC5)nc(OC[C@@]56CCCN5C[C@H](F)C6)nc4c3F)c12 ; CCc1c(F)ccc2cc(O)cc(-c3ncc4c(NS(=O)(=O)C5CCCNCC5)nc(OC[C@@]56CCCN5C[C@H](F)C6)nc4c3F)c12] 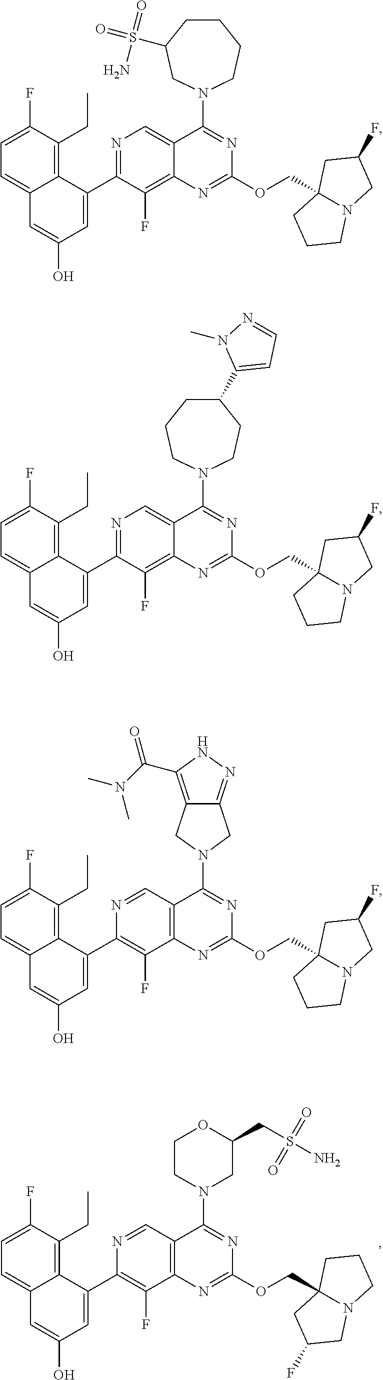 [SMILES: CCc1c(F)ccc2cc(O)cc(-c3ncc4c(N5CCCCC(S(N)(=O)=O)C5)nc(OC[C@@]56CCCN5C[C@H](F)C6)nc4c3F)c12 ; CCc1c(F)ccc2cc(O)cc(-c3ncc4c(N5CCC[C@H](c6ccnn6C)CC5)nc(OC[C@@]56CCCN5C[C@H](F)C6)nc4c3F)c12 ; CCc1c(F)ccc2cc(O)cc(-c3ncc4c(N5Cc6n[nH]c(C(=O)N(C)C)c6C5)nc(OC[C@@]56CCCN5C[C@H](F)C6)nc4c3F)c12 ; CCc1c(F)ccc2cc(O)cc(-c3ncc4c(N5CCO[C@@H](CS(N)(=O)=O)C5)nc(OC[C@@]56CCCN5C[C@H](F)C6)nc4c3F)c12] 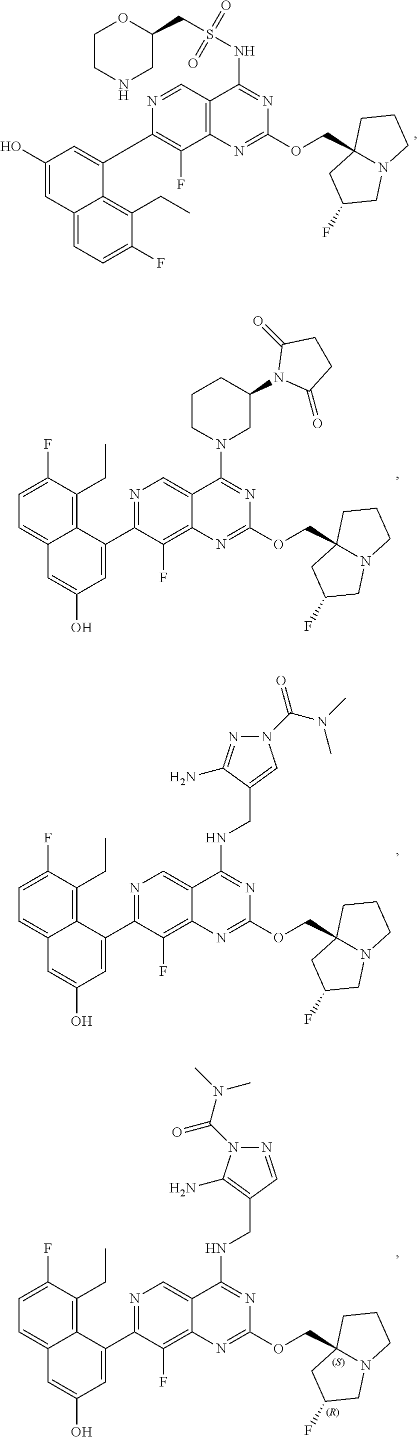 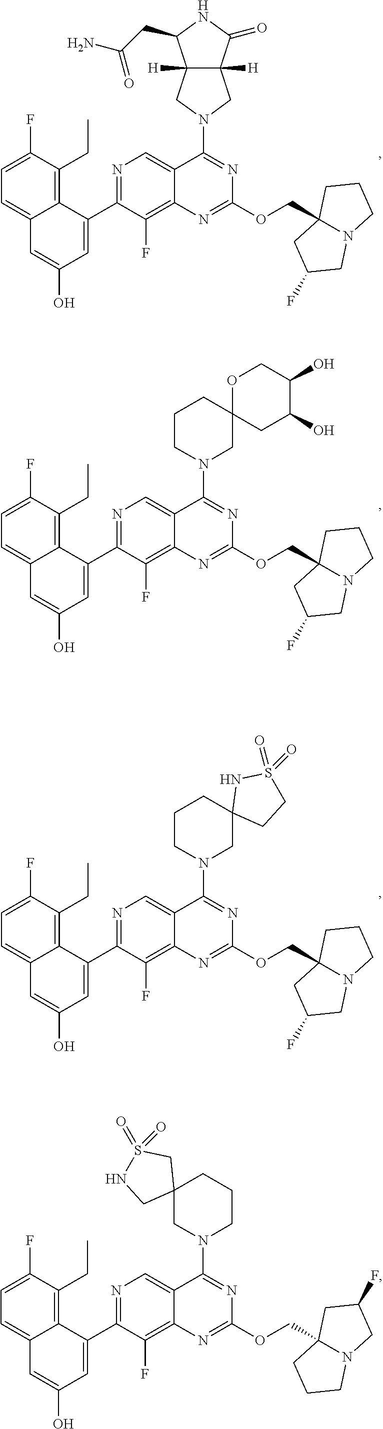 [SMILES: CCc1c(F)ccc2cc(O)cc(-c3ncc4c(N5C[C@@H]6[C@@H](CC(N)=O)NC(=O)[C@@H]6C5)nc(OC[C@@]56CCCN5C[C@H](F)C6)nc4c3F)c12 ; CCc1c(F)ccc2cc(O)cc(-c3ncc4c(N5CCCC6(C[C@H](O)[C@H](O)CO6)C5)nc(OC[C@@]56CCCN5C[C@H](F)C6)nc4c3F)c12 ; CCc1c(F)ccc2cc(O)cc(-c3ncc4c(N5CCCC6(CCS(=O)(=O)N6)C5)nc(OC[C@@]56CCCN5C[C@H](F)C6)nc4c3F)c12 ; CCc1c(F)ccc2cc(O)cc(-c3ncc4c(N5CCCC6(CNS(=O)(=O)C6)C5)nc(OC[C@@]56CCCN5C[C@H](F)C6)nc4c3F)c12] 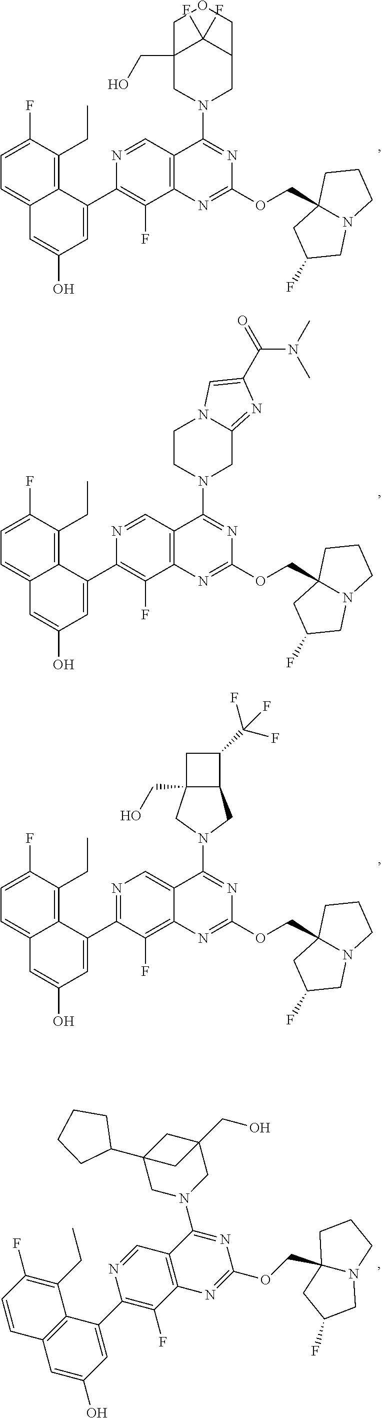 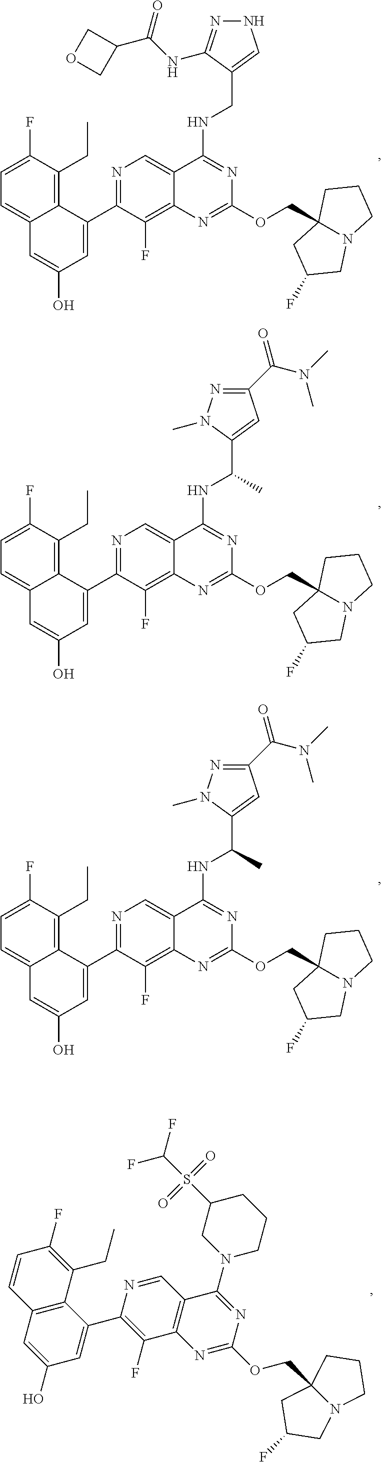 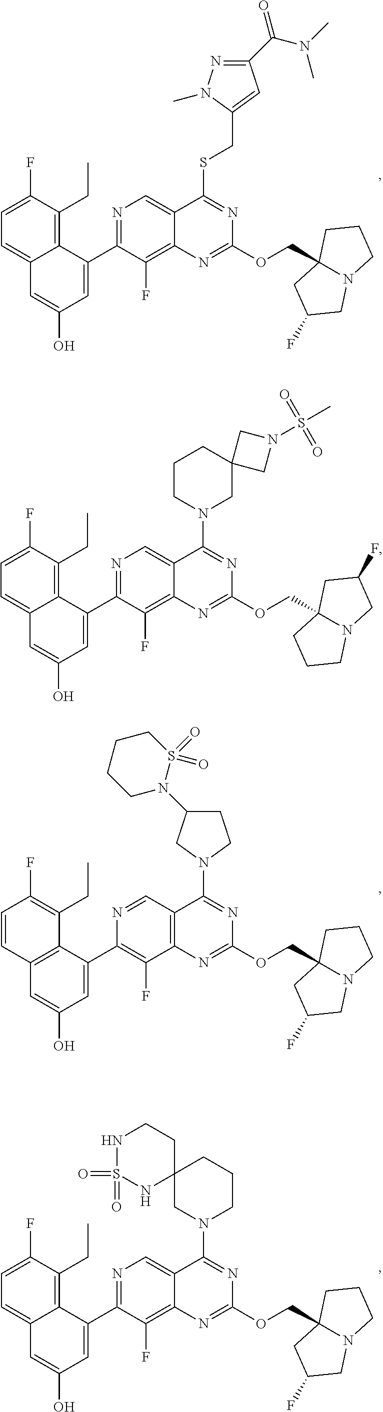 [SMILES: CCc1c(F)ccc2cc(O)cc(-c3ncc4c(SCc5cc(C(=O)N(C)C)nn5C)nc(OC[C@@]56CCCN5C[C@H](F)C6)nc4c3F)c12 ; CCc1c(F)ccc2cc(O)cc(-c3ncc4c(N5CCCC6(C5)CN(S(C)(=O)=O)C6)nc(OC[C@@]56CCCN5C[C@H](F)C6)nc4c3F)c12 ; CCc1c(F)ccc2cc(O)cc(-c3ncc4c(N5CCC(N6CCCCS6(=O)=O)C5)nc(OC[C@@]56CCCN5C[C@H](F)C6)nc4c3F)c12 ; CCc1c(F)ccc2cc(O)cc(-c3ncc4c(N5CCCC6(CCNS(=O)(=O)N6)C5)nc(OC[C@@]56CCCN5C[C@H](F)C6)nc4c3F)c12] 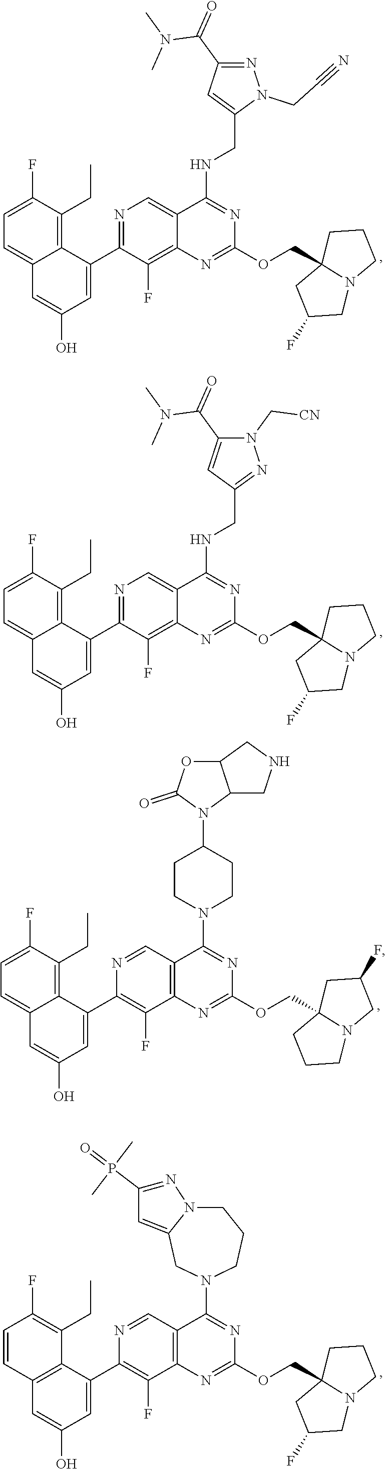 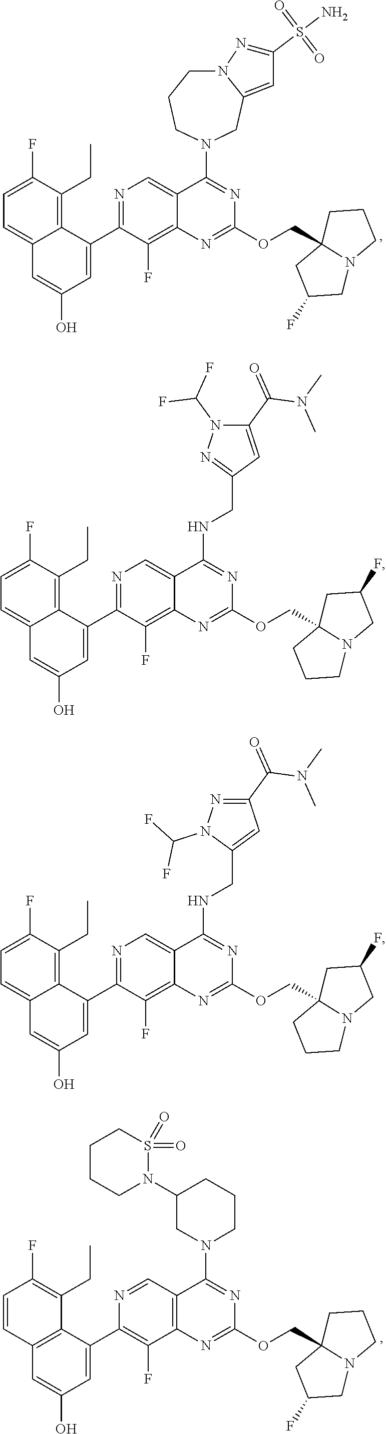 [SMILES: CCc1c(F)ccc2cc(O)cc(-c3ncc4c(N5CCCn6nc(S(N)(=O)=O)cc6C5)nc(OC[C@@]56CCCN5C[C@H](F)C6)nc4c3F)c12 ; CCc1c(F)ccc2cc(O)cc(-c3ncc4c(NCc5cc(C(=O)N(C)C)n(C(F)F)n5)nc(OC[C@@]56CCCN5C[C@H](F)C6)nc4c3F)c12 ; CCc1c(F)ccc2cc(O)cc(-c3ncc4c(NCc5cc(C(=O)N(C)C)nn5C(F)F)nc(OC[C@@]56CCCN5C[C@H](F)C6)nc4c3F)c12 ; CCc1c(F)ccc2cc(O)cc(-c3ncc4c(N5CCCC(N6CCCCS6(=O)=O)C5)nc(OC[C@@]56CCCN5C[C@H](F)C6)nc4c3F)c12] 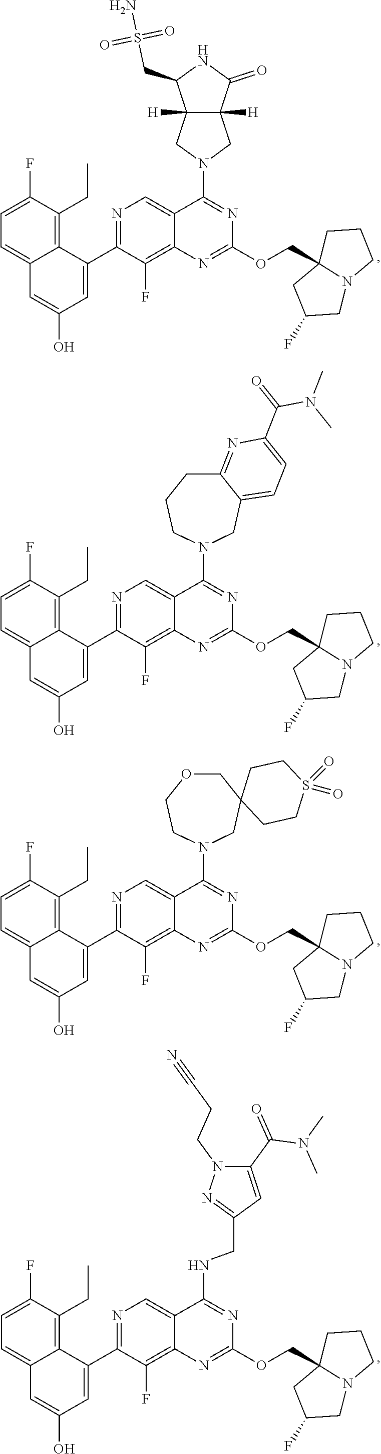 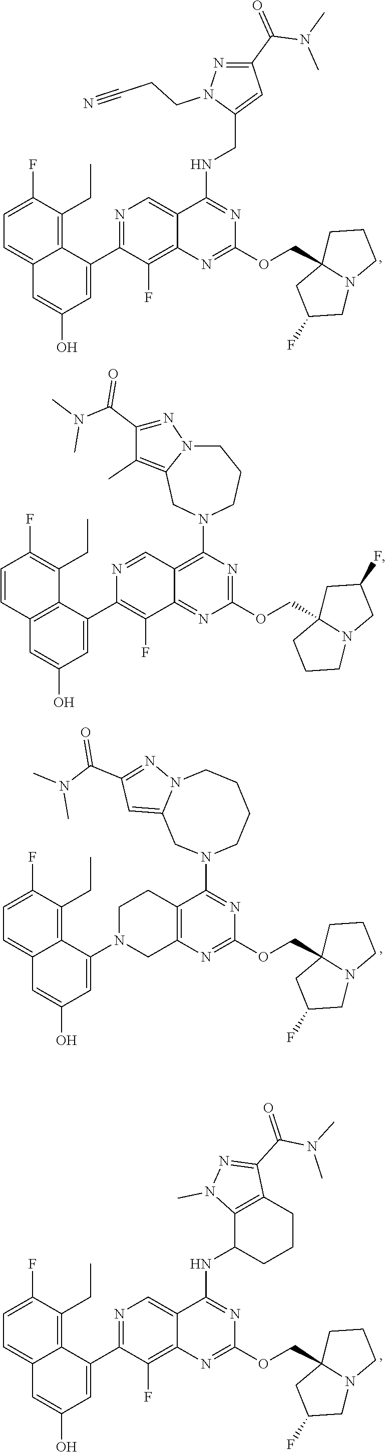 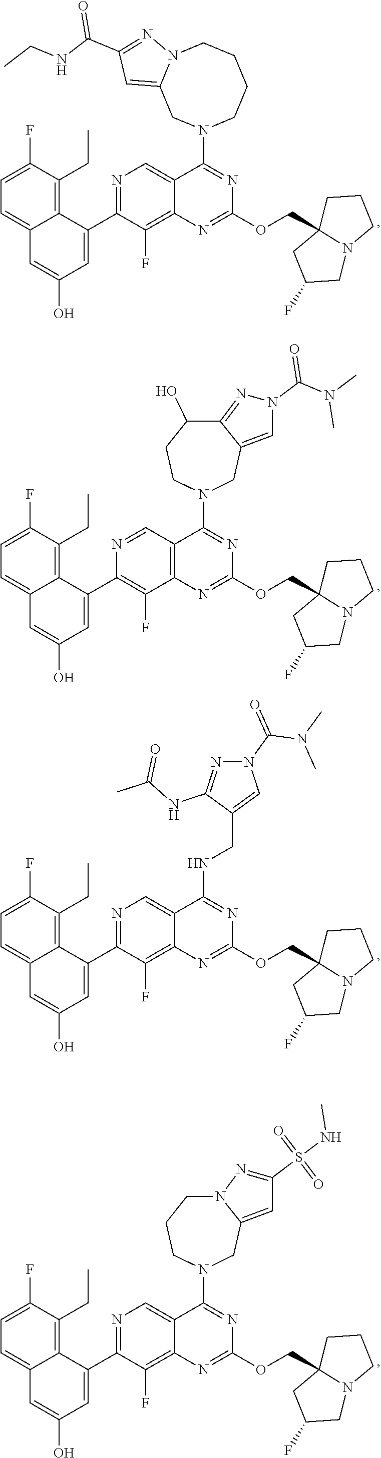 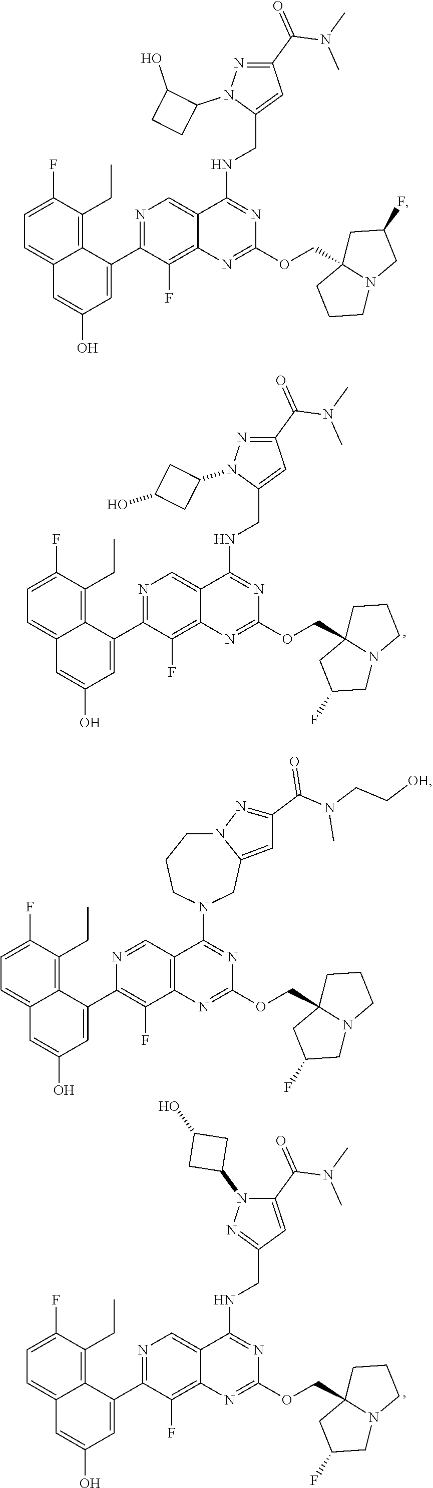 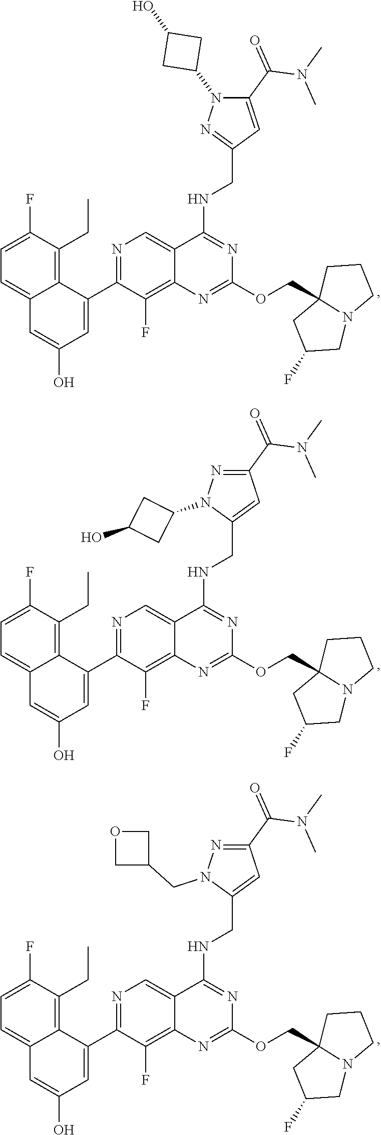 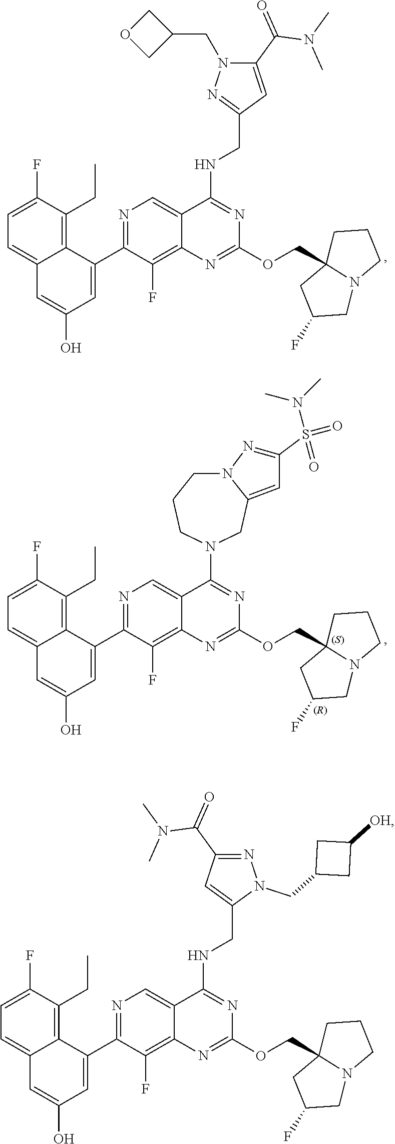 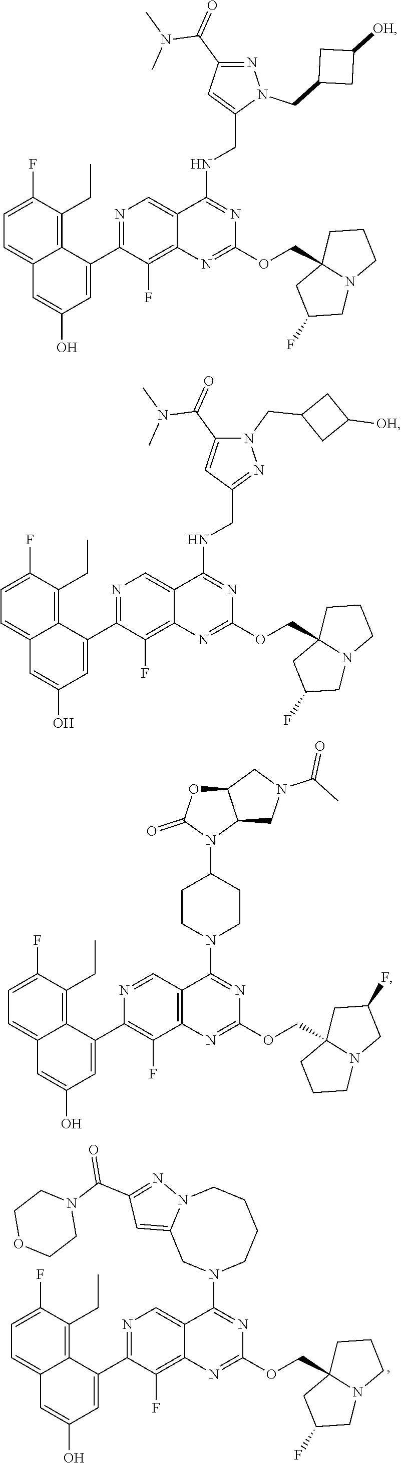 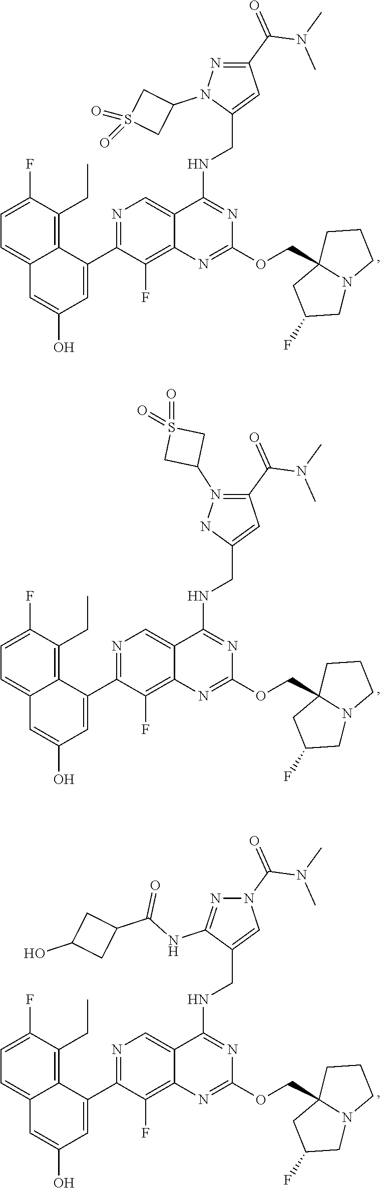 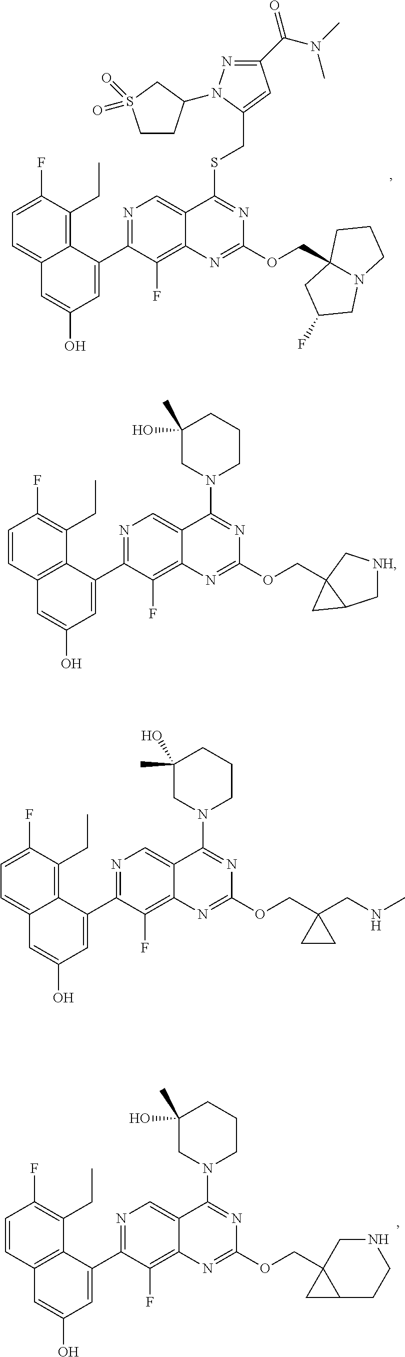 [SMILES: CCc1c(F)ccc2cc(O)cc(-c3ncc4c(SCc5cc(C(=O)N(C)C)nn5C5CCS(=O)(=O)C5)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OCC56CNCC5C6)nc4c3F)c12 ; CCc1c(F)ccc2cc(O)cc(-c3ncc4c(N5CCC[C@@](C)(O)C5)nc(OCC5(CNC)CC5)nc4c3F)c12 ; CCc1c(F)ccc2cc(O)cc(-c3ncc4c(N5CCC[C@@](C)(O)C5)nc(OCC56CNCCC5C6)nc4c3F)c12] 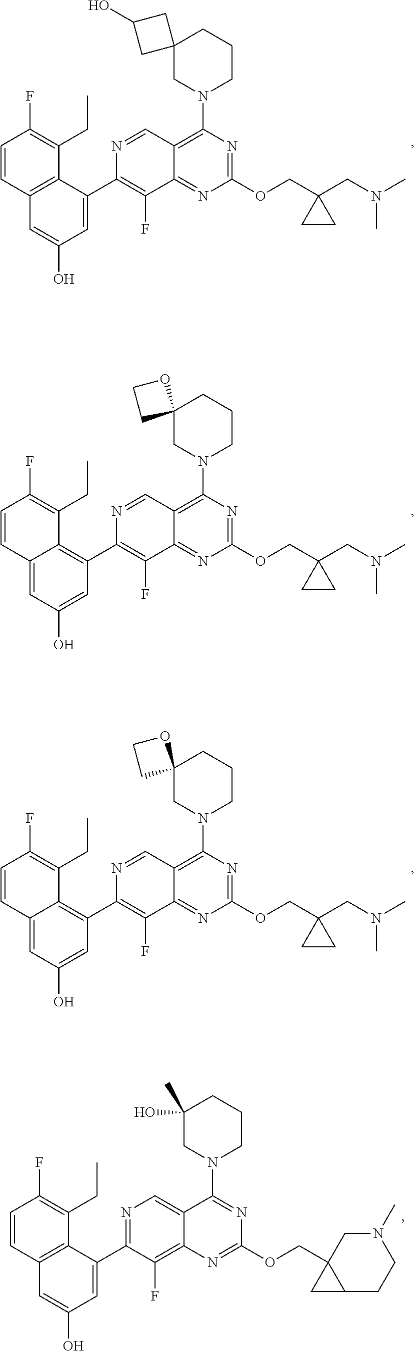 [SMILES: CCc1c(F)ccc2cc(O)cc(-c3ncc4c(N5CCCC6(CC(O)C6)C5)nc(OCC5(CN(C)C)CC5)nc4c3F)c12 ; CCc1c(F)ccc2cc(O)cc(-c3ncc4c(N5CCC[C@]6(CCO6)C5)nc(OCC5(CN(C)C)CC5)nc4c3F)c12 ; CCc1c(F)ccc2cc(O)cc(-c3ncc4c(N5CCC[C@@]6(CCO6)C5)nc(OCC5(CN(C)C)CC5)nc4c3F)c12 ; CCc1c(F)ccc2cc(O)cc(-c3ncc4c(N5CCC[C@@](C)(O)C5)nc(OCC56CC5CCN(C)C6)nc4c3F)c12] 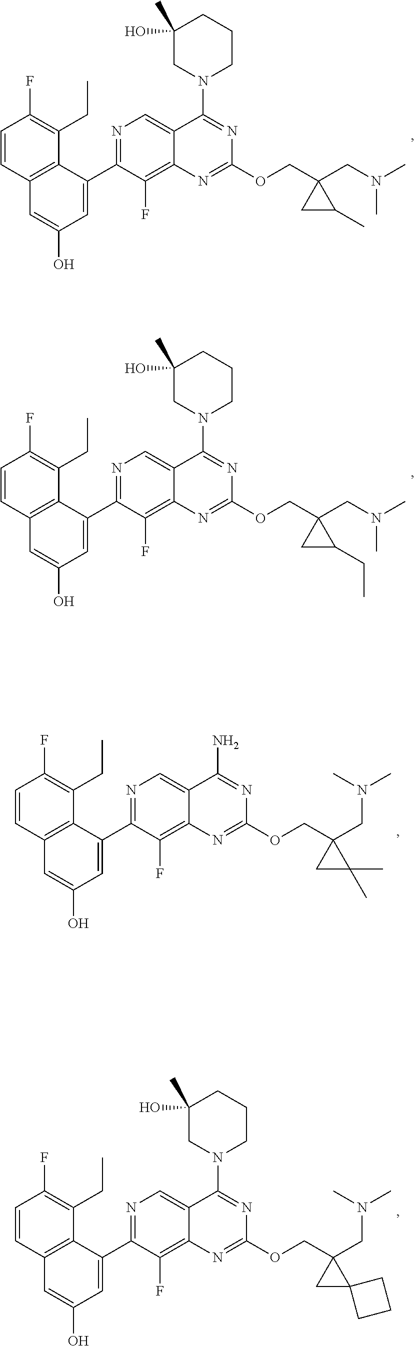 [SMILES: CCc1c(F)ccc2cc(O)cc(-c3ncc4c(N5CCC[C@@](C)(O)C5)nc(OCC5(CN(C)C)CC5C)nc4c3F)c12 ; CCc1c(F)ccc2cc(O)cc(-c3ncc4c(N5CCC[C@@](C)(O)C5)nc(OCC5(CN(C)C)CC5CC)nc4c3F)c12 ; CCc1c(F)ccc2cc(O)cc(-c3ncc4c(N)nc(OCC5(CN(C)C)CC5(C)C)nc4c3F)c12 ; CCc1c(F)ccc2cc(O)cc(-c3ncc4c(N5CCC[C@@](C)(O)C5)nc(OCC5(CN(C)C)CC56CCC6)nc4c3F)c12] 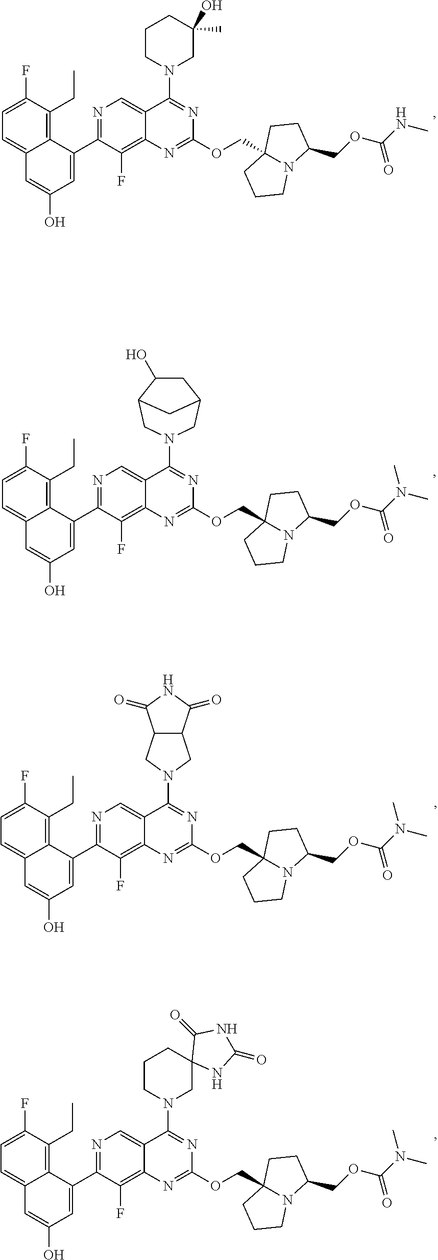 [SMILES: CCc1c(F)ccc2cc(O)cc(-c3ncc4c(N5CCC[C@@](C)(O)C5)nc(OC[C@@]56CCCN5[C@H](COC(=O)NC)CC6)nc4c3F)c12 ; CCc1c(F)ccc2cc(O)cc(-c3ncc4c(N5CC6CC(O)C(C6)C5)nc(OC[C@]56CCCN5[C@H](COC(=O)N(C)C)CC6)nc4c3F)c12 ; CCc1c(F)ccc2cc(O)cc(-c3ncc4c(N5CC6C(=O)NC(=O)C6C5)nc(OC[C@]56CCCN5[C@H](COC(=O)N(C)C)CC6)nc4c3F)c12 ; CCc1c(F)ccc2cc(O)cc(-c3ncc4c(N5CCCC6(C5)NC(=O)NC6=O)nc(OC[C@]56CCCN5[C@H](COC(=O)N(C)C)CC6)nc4c3F)c12] 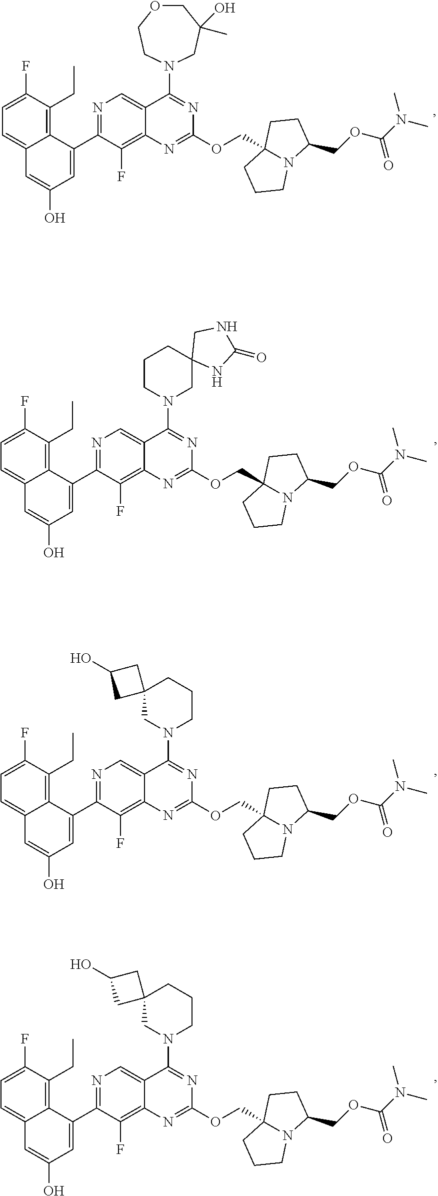 [SMILES: CCc1c(F)ccc2cc(O)cc(-c3ncc4c(N5CCOCC(C)(O)C5)nc(OC[C@@]56CCCN5[C@H](COC(=O)N(C)C)CC6)nc4c3F)c12 ; CCc1c(F)ccc2cc(O)cc(-c3ncc4c(N5CCCC6(CNC(=O)N6)C5)nc(OC[C@]56CCCN5[C@H](COC(=O)N(C)C)CC6)nc4c3F)c12 ; CCc1c(F)ccc2cc(O)cc(-c3ncc4c(N5CCC[C@]6(C5)C[C@H](O)C6)nc(OC[C@@]56CCCN5[C@H](COC(=O)N(C)C)CC6)nc4c3F)c12 ; CCc1c(F)ccc2cc(O)cc(-c3ncc4c(N5CCC[C@]6(C5)C[C@@H](O)C6)nc(OC[C@@]56CCCN5[C@H](COC(=O)N(C)C)CC6)nc4c3F)c12] 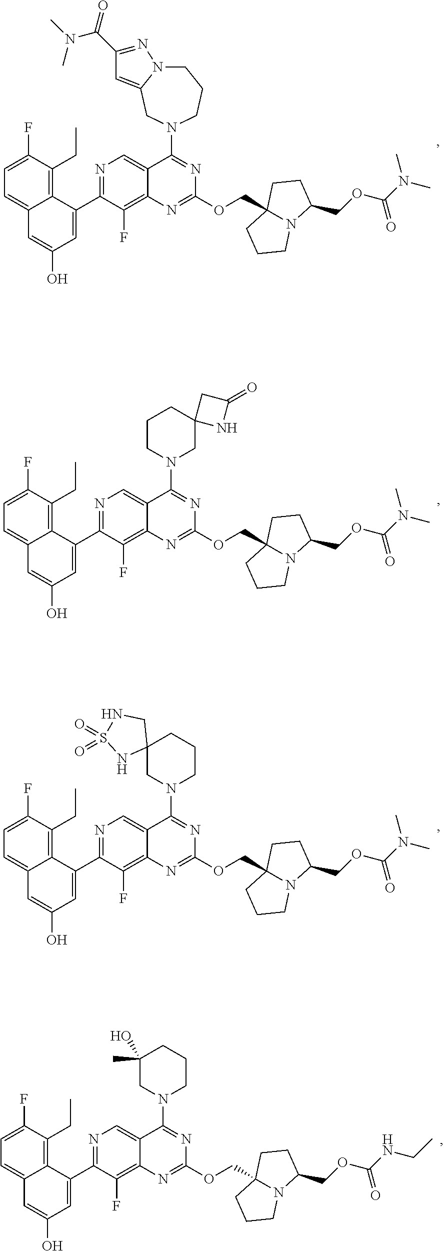 [SMILES: CCc1c(F)ccc2cc(O)cc(-c3ncc4c(N5CCCn6nc(C(=O)N(C)C)cc6C5)nc(OC[C@]56CCCN5[C@H](COC(=O)N(C)C)CC6)nc4c3F)c12 ; CCc1c(F)ccc2cc(O)cc(-c3ncc4c(N5CCCC6(CC(=O)N6)C5)nc(OC[C@]56CCCN5[C@H](COC(=O)N(C)C)CC6)nc4c3F)c12 ; CCc1c(F)ccc2cc(O)cc(-c3ncc4c(N5CCCC6(CNS(=O)(=O)N6)C5)nc(OC[C@]56CCCN5[C@H](COC(=O)N(C)C)CC6)nc4c3F)c12 ; CCNC(=O)OC[C@@H]1CC[C@]2(COc3nc(N4CCC[C@@](C)(O)C4)c4cnc(-c5cc(O)cc6ccc(F)c(CC)c56)c(F)c4n3)CCCN12] 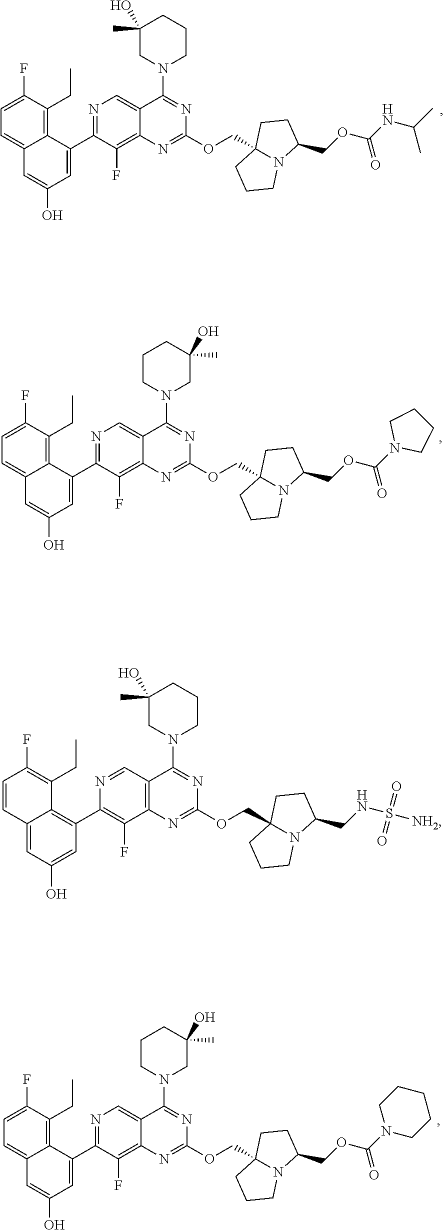 [SMILES: CCc1c(F)ccc2cc(O)cc(-c3ncc4c(N5CCC[C@@](C)(O)C5)nc(OC[C@@]56CCCN5[C@H](COC(=O)NC(C)C)CC6)nc4c3F)c12 ; CCc1c(F)ccc2cc(O)cc(-c3ncc4c(N5CCC[C@@](C)(O)C5)nc(OC[C@@]56CCCN5[C@H](COC(=O)N5CCCC5)CC6)nc4c3F)c12 ; CCc1c(F)ccc2cc(O)cc(-c3ncc4c(N5CCC[C@@](C)(O)C5)nc(OC[C@]56CCCN5[C@H](CNS(N)(=O)=O)CC6)nc4c3F)c12 ; CCc1c(F)ccc2cc(O)cc(-c3ncc4c(N5CCC[C@@](C)(O)C5)nc(OC[C@@]56CCCN5[C@H](COC(=O)N5CCCCC5)CC6)nc4c3F)c12] 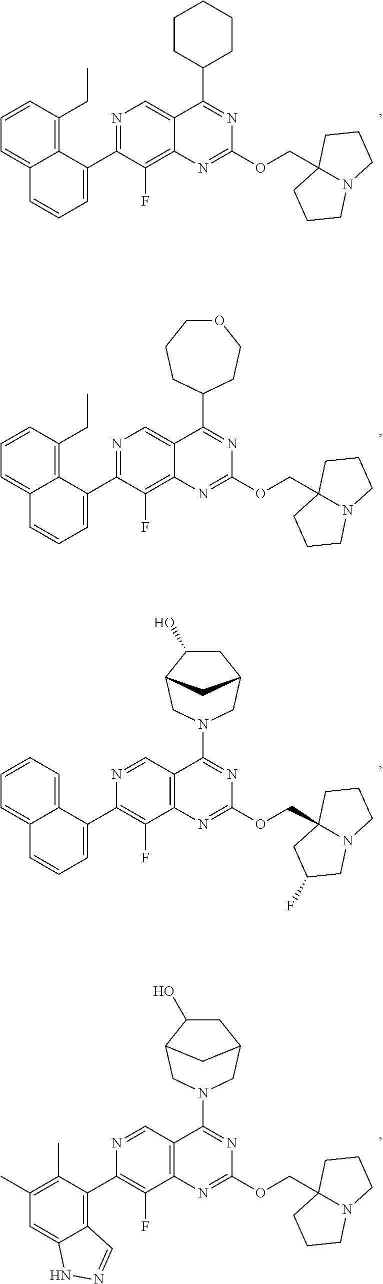 [SMILES: CCc1cccc2cccc(-c3ncc4c(C5CCCCC5)nc(OCC56CCCN5CCC6)nc4c3F)c12 ; CCc1cccc2cccc(-c3ncc4c(C5CCCOCC5)nc(OCC56CCCN5CCC6)nc4c3F)c12 ; O[C@@H]1C[C@H]2C[C@@H]1CN(c1nc(OC[C@@]34CCCN3C[C@H](F)C4)nc3c(F)c(-c4cccc5ccccc45)ncc13)C2 ; Cc1cc2[nH]ncc2c(-c2ncc3c(N4CC5CC(O)C(C5)C4)nc(OCC45CCCN4CCC5)nc3c2F)c1C] 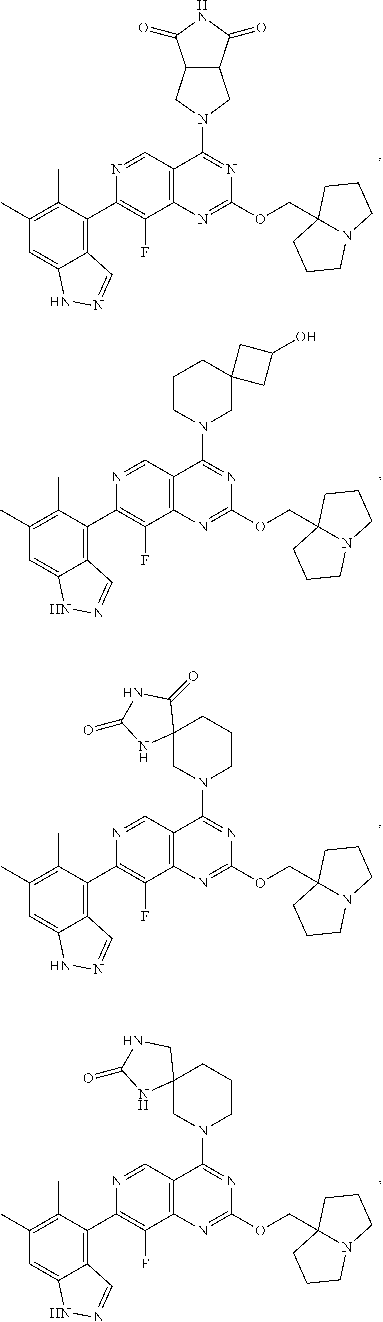 [SMILES: Cc1cc2[nH]ncc2c(-c2ncc3c(N4CC5C(=O)NC(=O)C5C4)nc(OCC45CCCN4CCC5)nc3c2F)c1C ; Cc1cc2[nH]ncc2c(-c2ncc3c(N4CCCC5(CC(O)C5)C4)nc(OCC45CCCN4CCC5)nc3c2F)c1C ; Cc1cc2[nH]ncc2c(-c2ncc3c(N4CCCC5(C4)NC(=O)NC5=O)nc(OCC45CCCN4CCC5)nc3c2F)c1C ; Cc1cc2[nH]ncc2c(-c2ncc3c(N4CCCC5(CNC(=O)N5)C4)nc(OCC45CCCN4CCC5)nc3c2F)c1C] 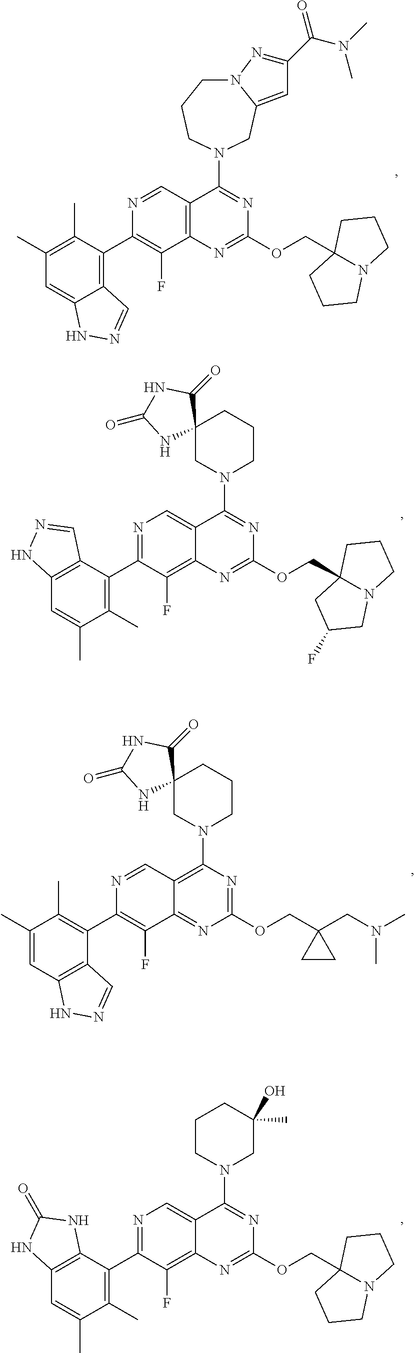 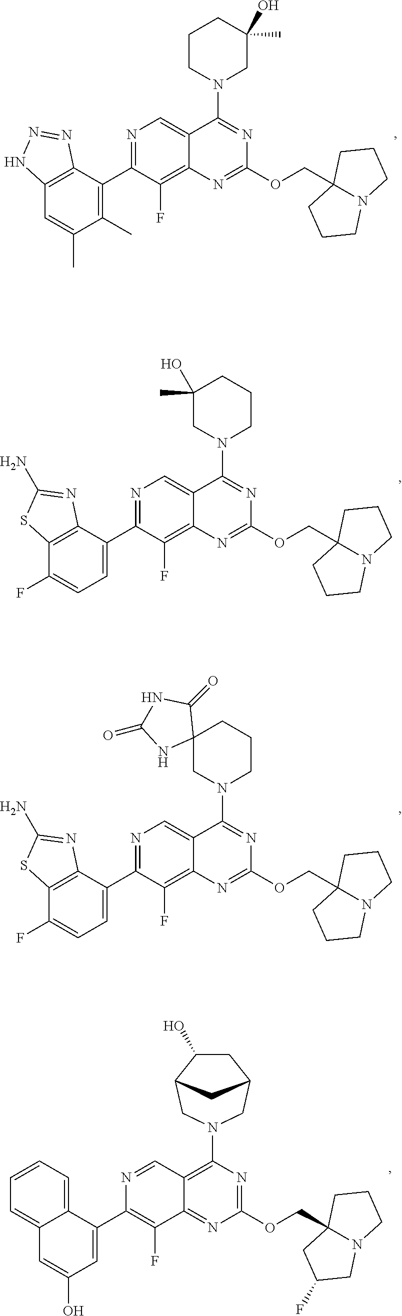 [SMILES: Cc1cc2[nH]nnc2c(-c2ncc3c(N4CCC[C@@](C)(O)C4)nc(OCC45CCCN4CCC5)nc3c2F)c1C ; C[C@@]1(O)CCCN(c2nc(OCC34CCCN3CCC4)nc3c(F)c(-c4ccc(F)c5sc(N)nc45)ncc23)C1 ; Nc1nc2c(-c3ncc4c(N5CCCC6(C5)NC(=O)NC6=O)nc(OCC56CCCN5CCC6)nc4c3F)ccc(F)c2s1 ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1] 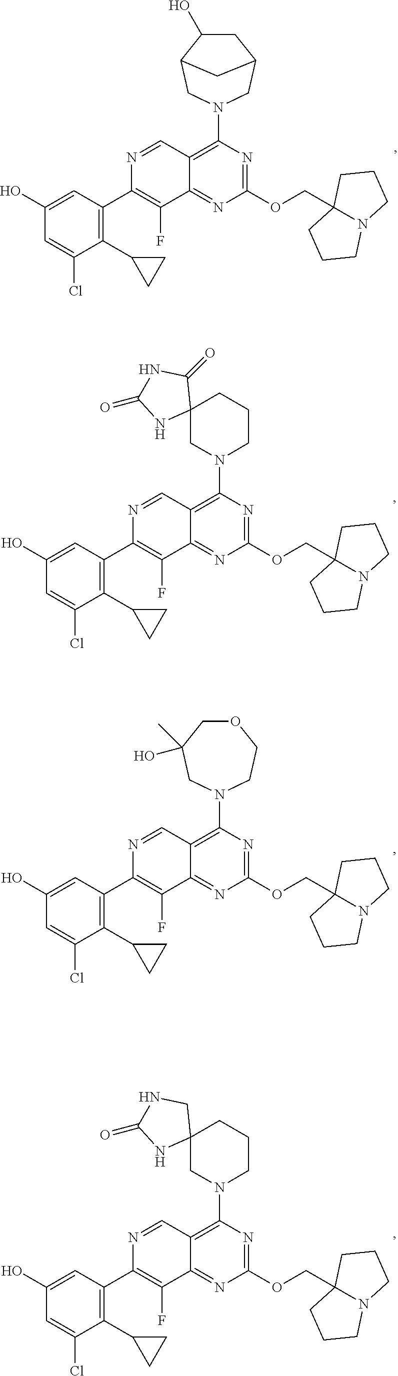 [SMILES: Oc1cc(Cl)c(C2CC2)c(-c2ncc3c(N4CC5CC(O)C(C5)C4)nc(OCC45CCCN4CCC5)nc3c2F)c1 ; O=C1NC(=O)C2(CCCN(c3nc(OCC45CCCN4CCC5)nc4c(F)c(-c5cc(O)cc(Cl)c5C5CC5)ncc34)C2)N1 ; CC1(O)COCCN(c2nc(OCC34CCCN3CCC4)nc3c(F)c(-c4cc(O)cc(Cl)c4C4CC4)ncc23)C1 ; O=C1NCC2(CCCN(c3nc(OCC45CCCN4CCC5)nc4c(F)c(-c5cc(O)cc(Cl)c5C5CC5)ncc34)C2)N1] 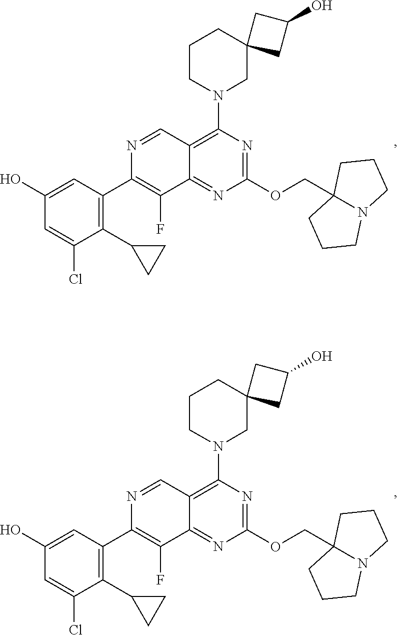 [SMILES: Oc1cc(Cl)c(C2CC2)c(-c2ncc3c(N4CCC[C@]5(C4)C[C@H](O)C5)nc(OCC45CCCN4CCC5)nc3c2F)c1 ; Oc1cc(Cl)c(C2CC2)c(-c2ncc3c(N4CCC[C@]5(C4)C[C@@H](O)C5)nc(OCC45CCCN4CCC5)nc3c2F)c1] 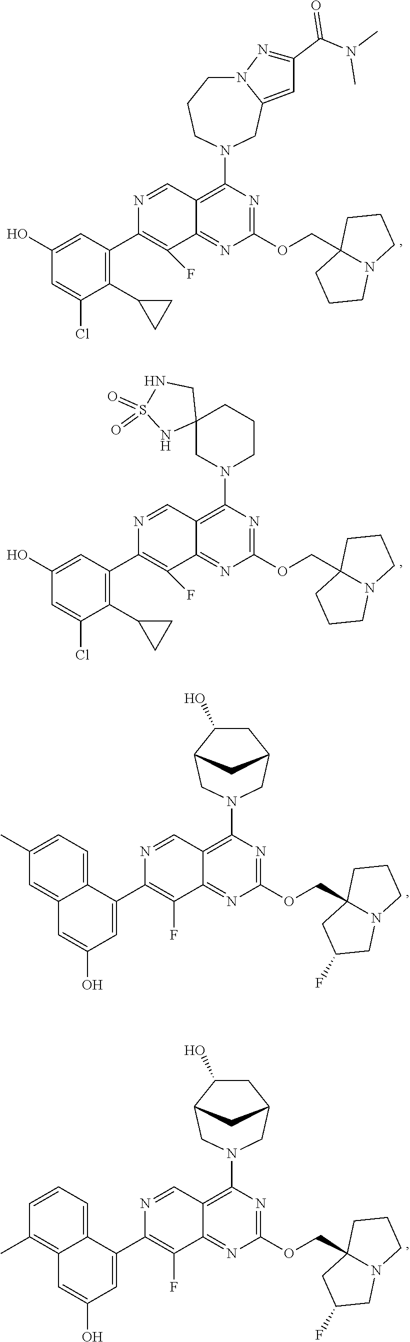 [SMILES: CN(C)C(=O)c1cc2n(n1)CCCN(c1nc(OCC34CCCN3CCC4)nc3c(F)c(-c4cc(O)cc(Cl)c4C4CC4)ncc13)C2 ; O=S1(=O)NCC2(CCCN(c3nc(OCC45CCCN4CCC5)nc4c(F)c(-c5cc(O)cc(Cl)c5C5CC5)ncc34)C2)N1 ; Cc1ccc2c(-c3ncc4c(N5C[C@@H]6C[C@H](C5)[C@H](O)C6)nc(OC[C@@]56CCCN5C[C@H](F)C6)nc4c3F)cc(O)cc2c1 ; Cc1cccc2c(-c3ncc4c(N5C[C@@H]6C[C@H](C5)[C@H](O)C6)nc(OC[C@@]56CCCN5C[C@H](F)C6)nc4c3F)cc(O)cc12] 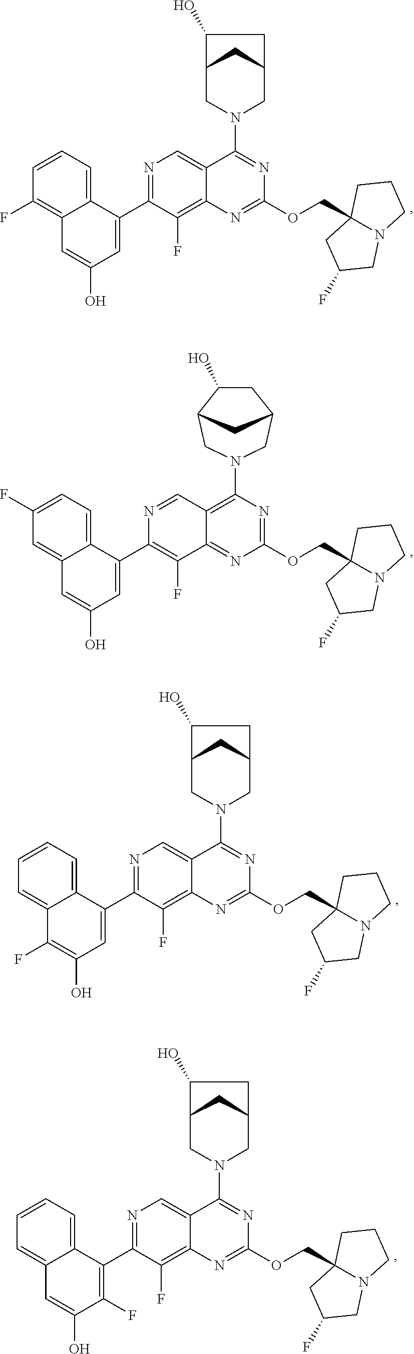 [SMILES: Oc1cc(-c2ncc3c(N4C[C@@H]5C[C@H](C4)[C@H](O)C5)nc(OC[C@@]45CCCN4C[C@H](F)C5)nc3c2F)c2cccc(F)c2c1 ; Oc1cc(-c2ncc3c(N4C[C@@H]5C[C@H](C4)[C@H](O)C5)nc(OC[C@@]45CCCN4C[C@H](F)C5)nc3c2F)c2ccc(F)cc2c1 ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1F ; Oc1cc2ccccc2c(-c2ncc3c(N4C[C@@H]5C[C@H](C4)[C@H](O)C5)nc(OC[C@@]45CCCN4C[C@H](F)C5)nc3c2F)c1F] 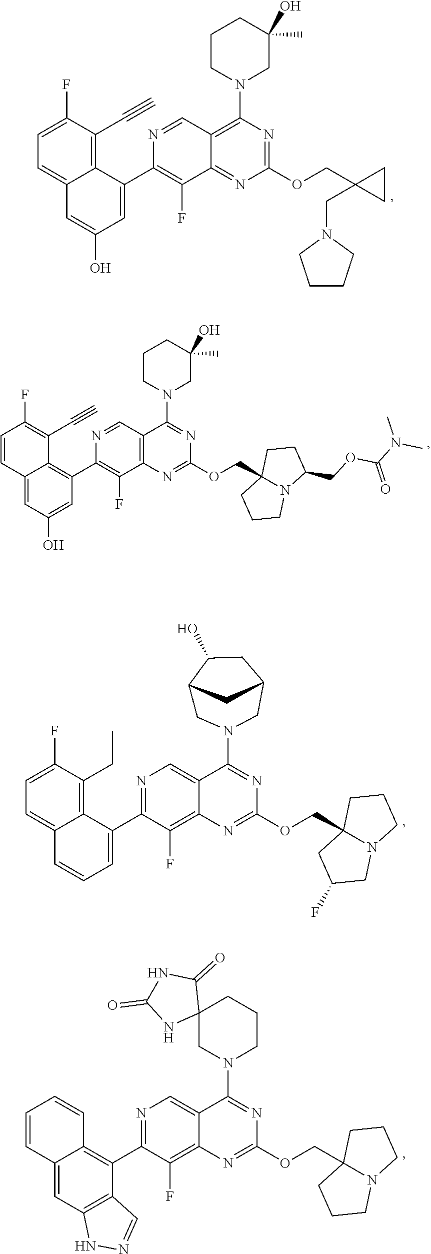 [SMILES: C#Cc1c(F)ccc2cc(O)cc(-c3ncc4c(N5CCC[C@@](C)(O)C5)nc(OCC5(CN6CCCC6)CC5)nc4c3F)c12 ; C#Cc1c(F)ccc2cc(O)cc(-c3ncc4c(N5CCC[C@@](C)(O)C5)nc(OC[C@]56CCCN5[C@H](COC(=O)N(C)C)CC6)nc4c3F)c12 ; CCc1c(F)ccc2cccc(-c3ncc4c(N5C[C@@H]6C[C@H](C5)[C@H](O)C6)nc(OC[C@@]56CCCN5C[C@H](F)C6)nc4c3F)c12 ; O=C1NC(=O)C2(CCCN(c3nc(OCC45CCCN4CCC5)nc4c(F)c(-c5c6ccccc6cc6[nH]ncc56)ncc34)C2)N1] 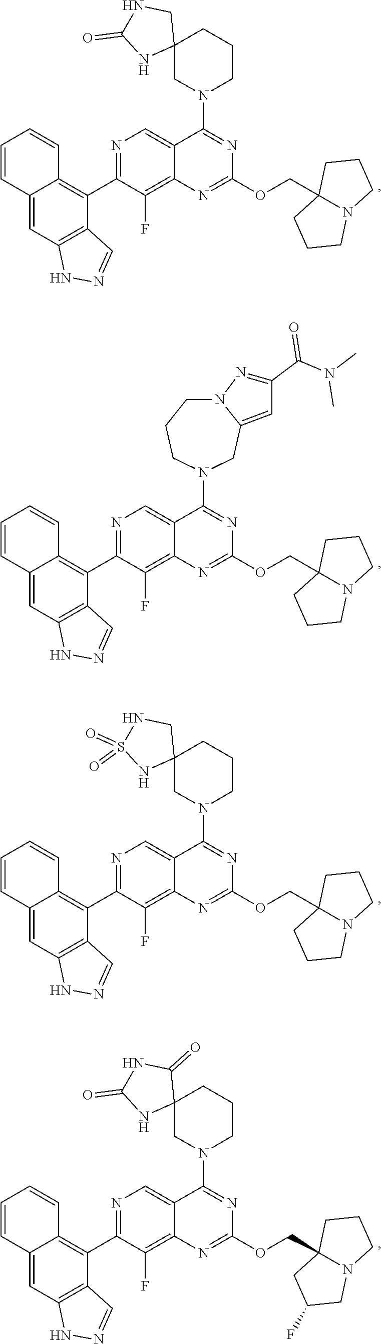 [SMILES: O=C1NCC2(CCCN(c3nc(OCC45CCCN4CCC5)nc4c(F)c(-c5c6ccccc6cc6[nH]ncc56)ncc34)C2)N1 ; CN(C)C(=O)c1cc2n(n1)CCCN(c1nc(OCC34CCCN3CCC4)nc3c(F)c(-c4c5ccccc5cc5[nH]ncc45)ncc13)C2 ; O=S1(=O)NCC2(CCCN(c3nc(OCC45CCCN4CCC5)nc4c(F)c(-c5c6ccccc6cc6[nH]ncc56)ncc34)C2)N1 ; O=C1NC(=O)C2(CCCN(c3nc(OC[C@@]45CCCN4C[C@H](F)C5)nc4c(F)c(-c5c6ccccc6cc6[nH]ncc56)ncc34)C2)N1] 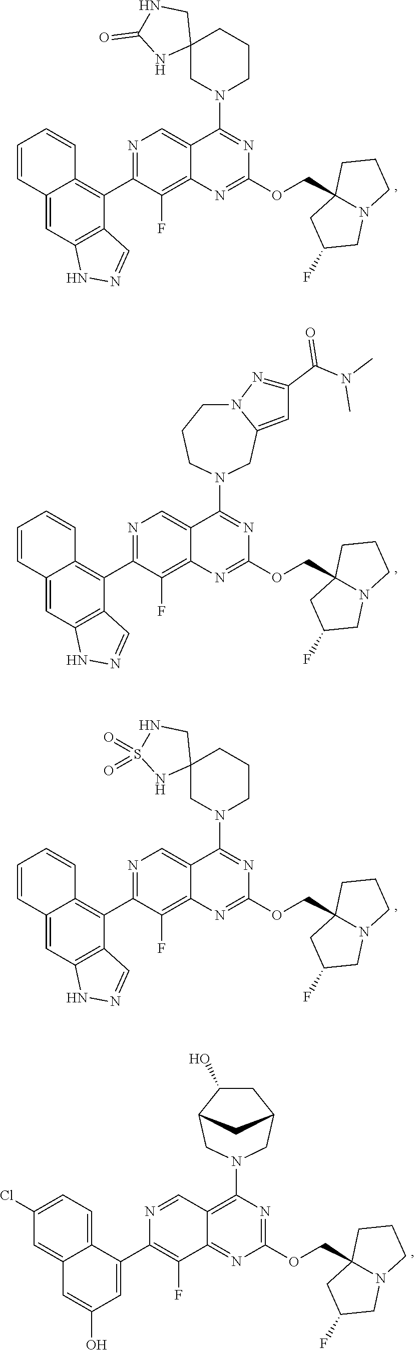 [SMILES: O=C1NCC2(CCCN(c3nc(OC[C@@]45CCCN4C[C@H](F)C5)nc4c(F)c(-c5c6ccccc6cc6[nH]ncc56)ncc34)C2)N1 ; CN(C)C(=O)c1cc2n(n1)CCCN(c1nc(OC[C@@]34CCCN3C[C@H](F)C4)nc3c(F)c(-c4c5ccccc5cc5[nH]ncc45)ncc13)C2 ; O=S1(=O)NCC2(CCCN(c3nc(OC[C@@]45CCCN4C[C@H](F)C5)nc4c(F)c(-c5c6ccccc6cc6[nH]ncc56)ncc34)C2)N1 ; Oc1cc(-c2ncc3c(N4C[C@@H]5C[C@H](C4)[C@H](O)C5)nc(OC[C@@]45CCCN4C[C@H](F)C5)nc3c2F)c2ccc(Cl)cc2c1] 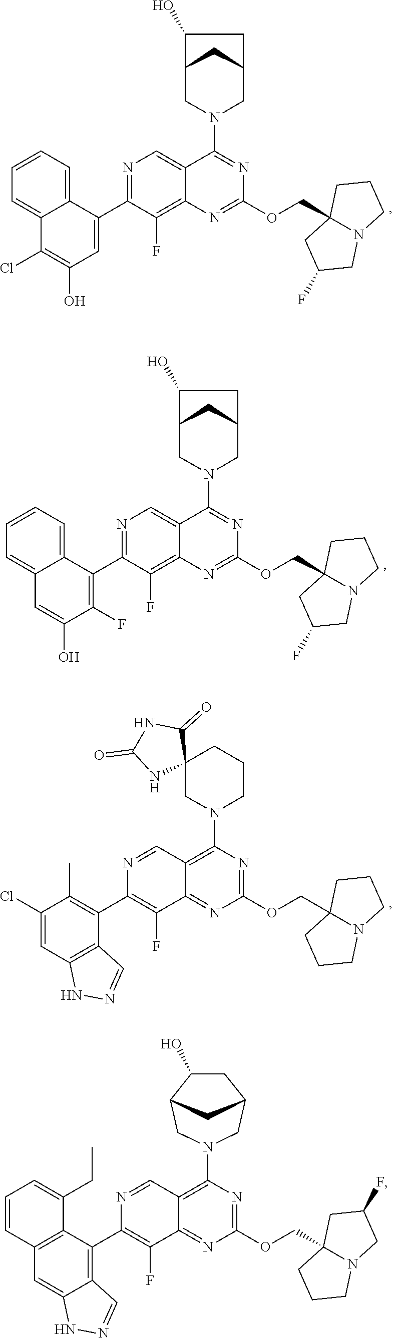 [SMILES: Oc1cc(-c2ncc3c(N4C[C@@H]5C[C@H](C4)[C@H](O)C5)nc(OC[C@@]45CCCN4C[C@H](F)C5)nc3c2F)c2ccccc2c1Cl ; Oc1cc2ccccc2c(-c2ncc3c(N4C[C@@H]5C[C@H](C4)[C@H](O)C5)nc(OC[C@@]45CCCN4C[C@H](F)C5)nc3c2F)c1F ; Cc1c(Cl)cc2[nH]ncc2c1-c1ncc2c(N3CCC[C@]4(C3)NC(=O)NC4=O)nc(OCC34CCCN3CCC4)nc2c1F ; CCc1cccc2cc3[nH]ncc3c(-c3ncc4c(N5C[C@@H]6C[C@H](C5)[C@H](O)C6)nc(OC[C@@]56CCCN5C[C@H](F)C6)nc4c3F)c12] 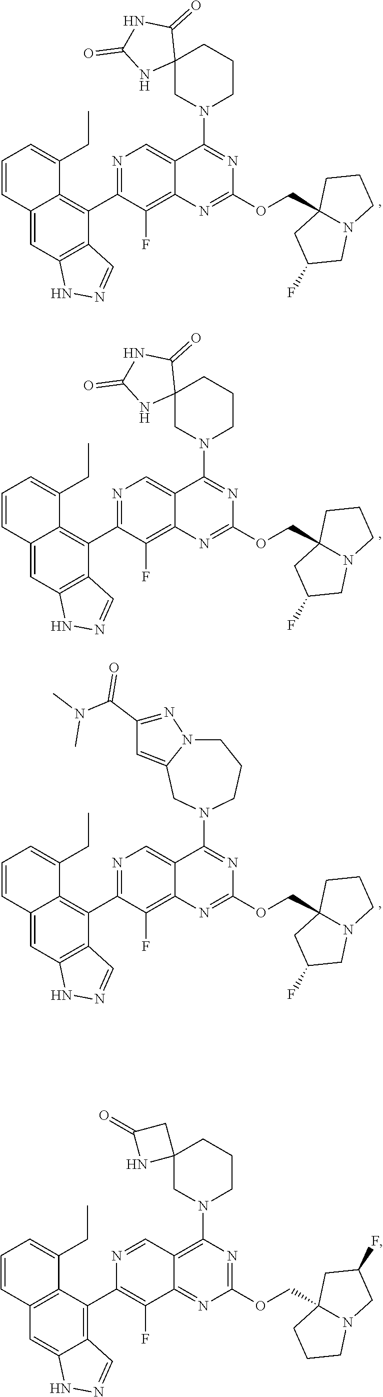 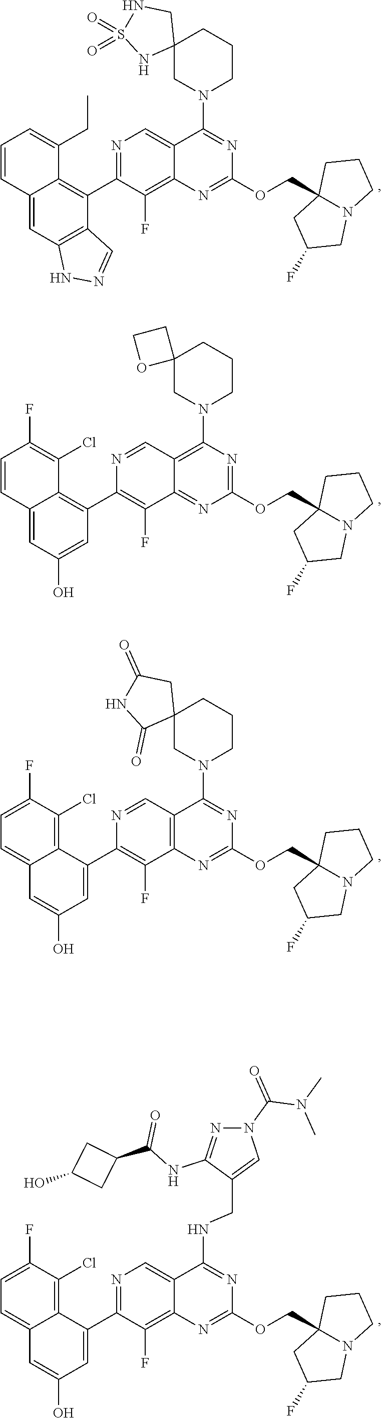 [SMILES: CCc1cccc2cc3[nH]ncc3c(-c3ncc4c(N5CCCC6(CNS(=O)(=O)N6)C5)nc(OC[C@@]56CCCN5C[C@H](F)C6)nc4c3F)c12 ; Oc1cc(-c2ncc3c(N4CCCC5(CCO5)C4)nc(OC[C@@]45CCCN4C[C@H](F)C5)nc3c2F)c2c(Cl)c(F)ccc2c1 ; O=C1CC2(CCCN(c3nc(OC[C@@]45CCCN4C[C@H](F)C5)nc4c(F)c(-c5cc(O)cc6ccc(F)c(Cl)c56)ncc34)C2)C(=O)N1 ; CN(C)C(=O)n1cc(CNc2nc(OC[C@@]34CCCN3C[C@H](F)C4)nc3c(F)c(-c4cc(O)cc5ccc(F)c(Cl)c45)ncc23)c(NC(=O)[C@H]2C[C@H](O)C2)n1] 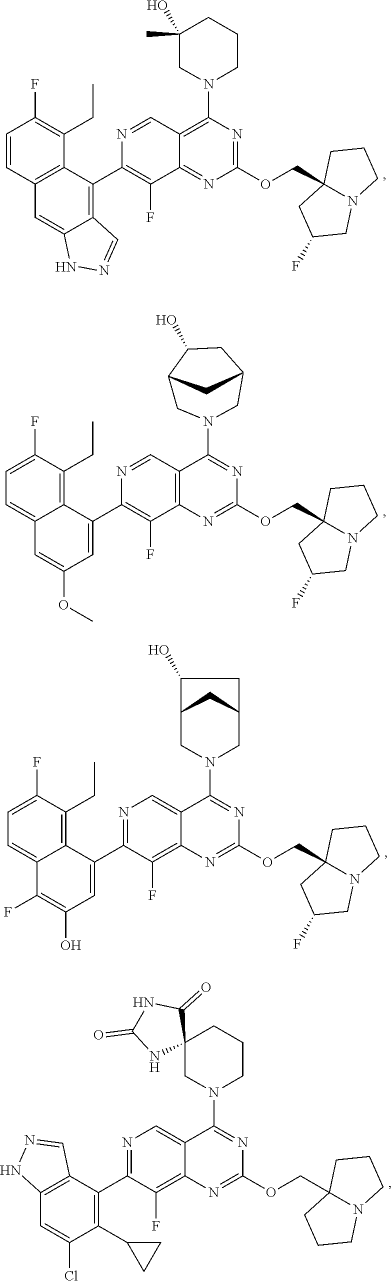 [SMILES: CCc1c(F)ccc2cc3[nH]ncc3c(-c3ncc4c(N5CCC[C@@](C)(O)C5)nc(OC[C@@]56CCCN5C[C@H](F)C6)nc4c3F)c12 ; CCc1c(F)ccc2cc(OC)cc(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2c(F)c(O)cc(-c3ncc4c(N5C[C@@H]6C[C@H](C5)[C@H](O)C6)nc(OC[C@@]56CCCN5C[C@H](F)C6)nc4c3F)c12 ; O=C1NC(=O)[C@]2(CCCN(c3nc(OCC45CCCN4CCC5)nc4c(F)c(-c5c(C6CC6)c(Cl)cc6[nH]ncc56)ncc34)C2)N1] 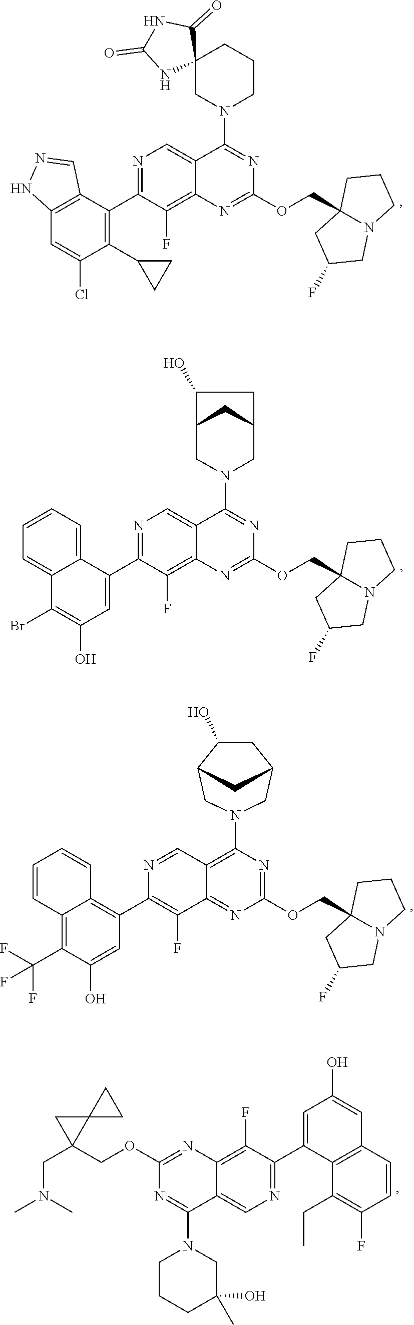 [SMILES: O=C1NC(=O)[C@]2(CCCN(c3nc(OC[C@@]45CCCN4C[C@H](F)C5)nc4c(F)c(-c5c(C6CC6)c(Cl)cc6[nH]ncc56)ncc34)C2)N1 ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1Br ; Oc1cc(-c2ncc3c(N4C[C@@H]5C[C@H](C4)[C@H](O)C5)nc(OC[C@@]45CCCN4C[C@H](F)C5)nc3c2F)c2ccccc2c1C(F)(F)F ; CCc1c(F)ccc2cc(O)cc(-c3ncc4c(N5CCC[C@@](C)(O)C5)nc(OCC5(CN(C)C)CC56CC6)nc4c3F)c12] 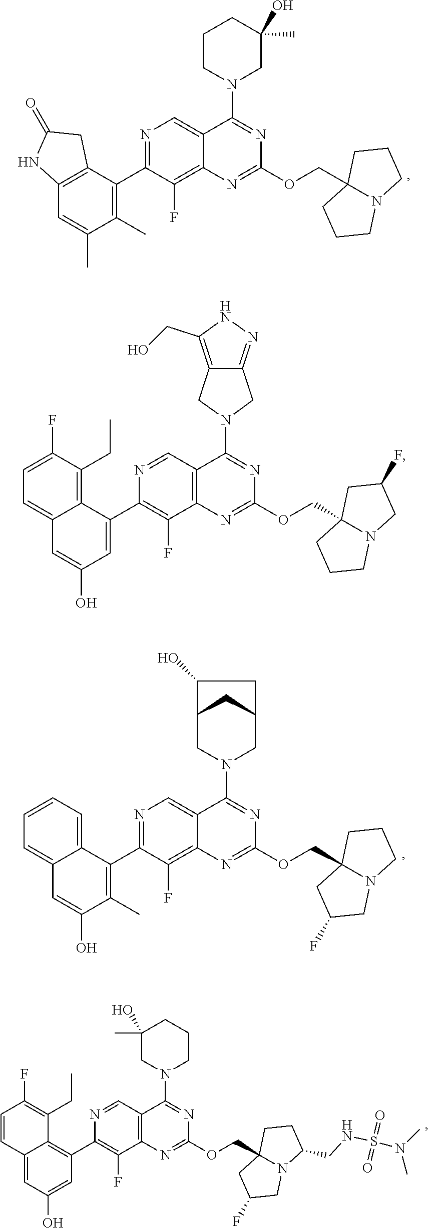 [SMILES: Cc1cc2c(c(-c3ncc4c(N5CCC[C@@](C)(O)C5)nc(OCC56CCCN5CCC6)nc4c3F)c1C)CC(=O)N2 ; CCc1c(F)ccc2cc(O)cc(-c3ncc4c(N5Cc6n[nH]c(CO)c6C5)nc(OC[C@@]56CCCN5C[C@H](F)C6)nc4c3F)c12 ; Cc1c(O)cc2ccccc2c1-c1ncc2c(N3C[C@@H]4C[C@H](C3)[C@H](O)C4)nc(OC[C@@]34CCCN3C[C@H](F)C4)nc2c1F ; CCc1c(F)ccc2cc(O)cc(-c3ncc4c(N5CCC[C@@](C)(O)C5)nc(OC[C@@]56CC[C@H](CNS(=O)(=O)N(C)C)N5C[C@H](F)C6)nc4c3F)c12] 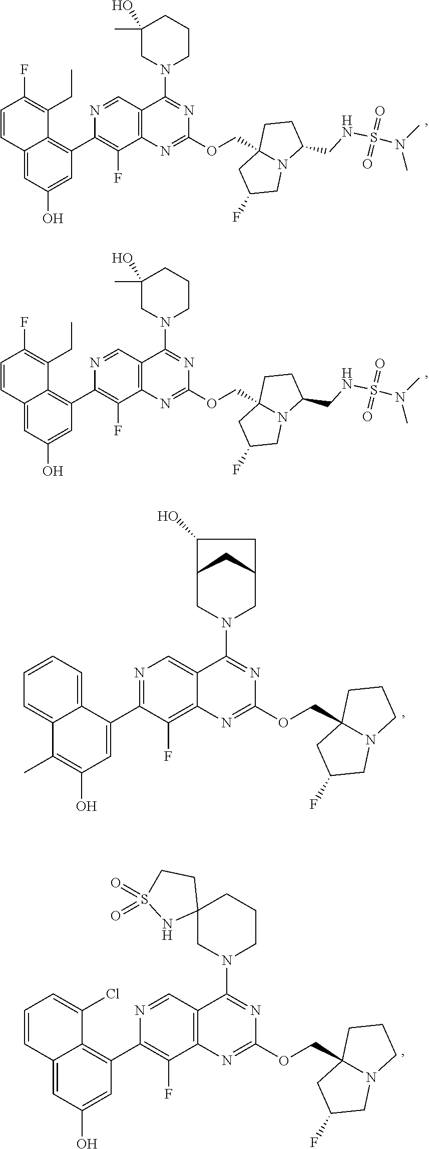 [SMILES: CCc1c(F)ccc2cc(O)cc(-c3ncc4c(N5CCC[C@@](C)(O)C5)nc(OC[C@]56CC[C@H](CNS(=O)(=O)N(C)C)N5C[C@H](F)C6)nc4c3F)c12 ; CCc1c(F)ccc2cc(O)cc(-c3ncc4c(N5CCC[C@@](C)(O)C5)nc(OC[C@]56CC[C@@H](CNS(=O)(=O)N(C)C)N5C[C@H](F)C6)nc4c3F)c12 ; Cc1c(O)cc(-c2ncc3c(N4C[C@@H]5C[C@H](C4)[C@H](O)C5)nc(OC[C@@]45CCCN4C[C@H](F)C5)nc3c2F)c2ccccc12 ; O=S1(=O)CCC2(CCCN(c3nc(OC[C@@]45CCCN4C[C@H](F)C5)nc4c(F)c(-c5cc(O)cc6cccc(Cl)c56)ncc34)C2)N1] 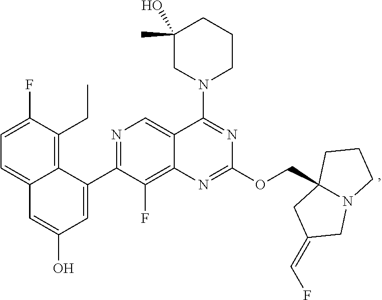 [SMILES: CCc1c(F)ccc2cc(O)cc(-c3ncc4c(N5CCC[C@@](C)(O)C5)nc(OC[C@@]56CCCN5C/C(=C\F)C6)nc4c3F)c12] 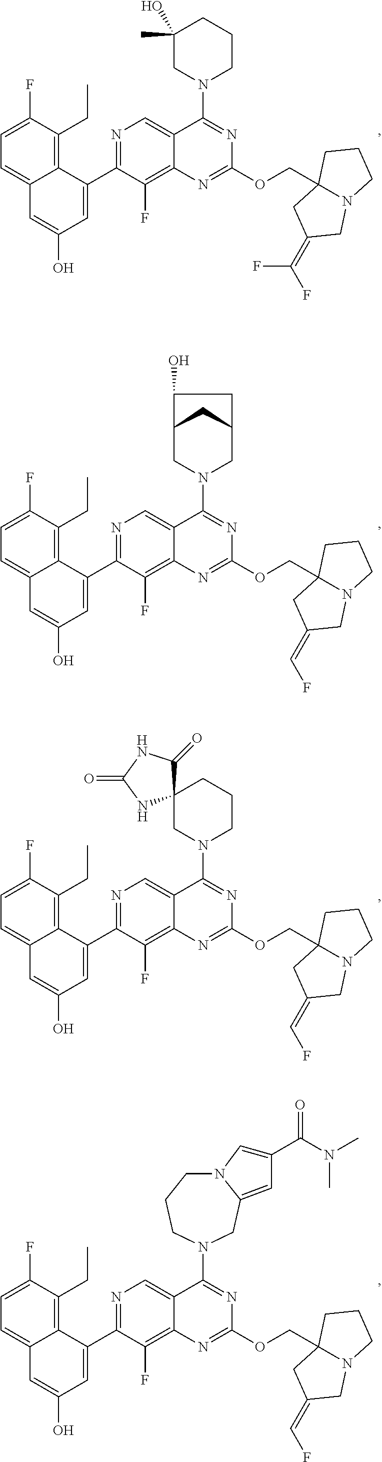 [SMILES: CCc1c(F)ccc2cc(O)cc(-c3ncc4c(N5CCC[C@@](C)(O)C5)nc(OCC56CCCN5CC(=C(F)F)C6)nc4c3F)c12 ; CCc1c(F)ccc2cc(O)cc(-c3ncc4c(N5C[C@@H]6C[C@H](C5)[C@H](O)C6)nc(OCC56CCCN5C/C(=C\F)C6)nc4c3F)c12 ; CCc1c(F)ccc2cc(O)cc(-c3ncc4c(N5CCC[C@]6(C5)NC(=O)NC6=O)nc(OCC56CCCN5C/C(=C\F)C6)nc4c3F)c12 ; CCc1c(F)ccc2cc(O)cc(-c3ncc4c(N5CCCn6cc(C(=O)N(C)C)cc6C5)nc(OCC56CCCN5C/C(=C\F)C6)nc4c3F)c12] 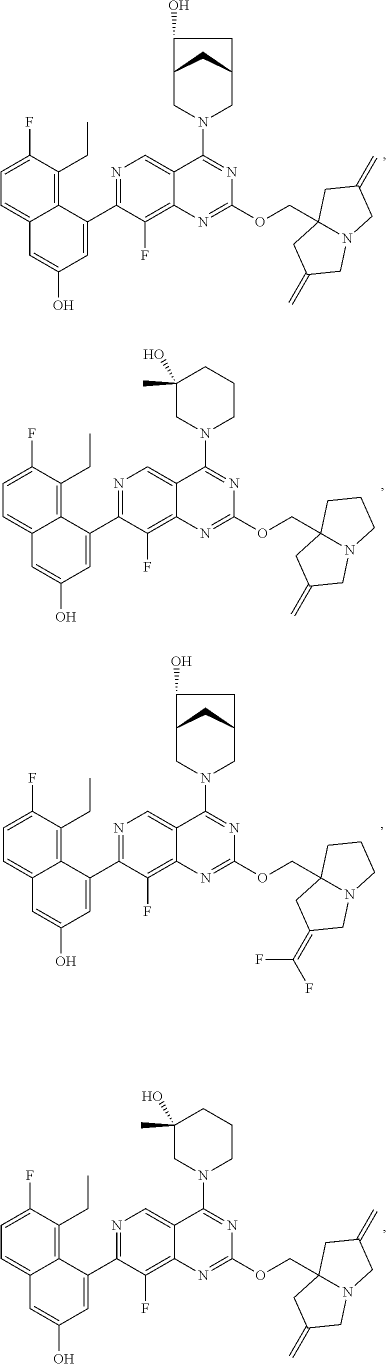 [SMILES: C=C1CN2CC(=C)CC2(COc2nc(N3C[C@@H]4C[C@H](C3)[C@H](O)C4)c3cnc(-c4cc(O)cc5ccc(F)c(CC)c45)c(F)c3n2)C1 ; C=C1CN2CCCC2(COc2nc(N3CCC[C@@](C)(O)C3)c3cnc(-c4cc(O)cc5ccc(F)c(CC)c45)c(F)c3n2)C1 ; CCc1c(F)ccc2cc(O)cc(-c3ncc4c(N5C[C@@H]6C[C@H](C5)[C@H](O)C6)nc(OCC56CCCN5CC(=C(F)F)C6)nc4c3F)c12 ; C=C1CN2CC(=C)CC2(COc2nc(N3CCC[C@@](C)(O)C3)c3cnc(-c4cc(O)cc5ccc(F)c(CC)c45)c(F)c3n2)C1] 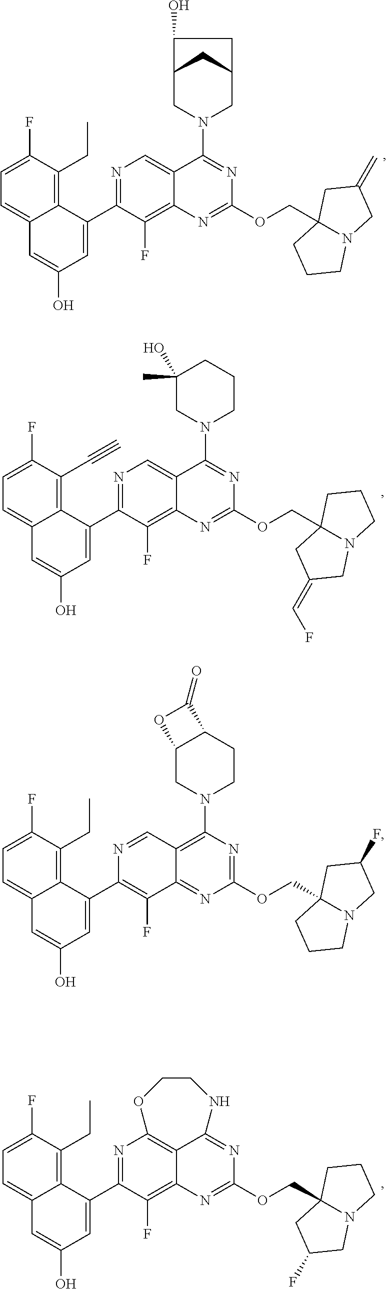 [SMILES: C=C1CN2CCCC2(COc2nc(N3C[C@@H]4C[C@H](C3)[C@H](O)C4)c3cnc(-c4cc(O)cc5ccc(F)c(CC)c45)c(F)c3n2)C1 ; C#Cc1c(F)ccc2cc(O)cc(-c3ncc4c(N5CCC[C@@](C)(O)C5)nc(OCC56CCCN5C/C(=C\F)C6)nc4c3F)c12 ; CCc1c(F)ccc2cc(O)cc(-c3ncc4c(N5CC[C@H]6C(=O)O[C@H]6C5)nc(OC[C@@]56CCCN5C[C@H](F)C6)nc4c3F)c12 ; CCc1c(F)ccc2cc(O)cc(-c3nc4c5c(nc(OC[C@@]67CCCN6C[C@H](F)C7)nc5c3F)NCCO4)c12] 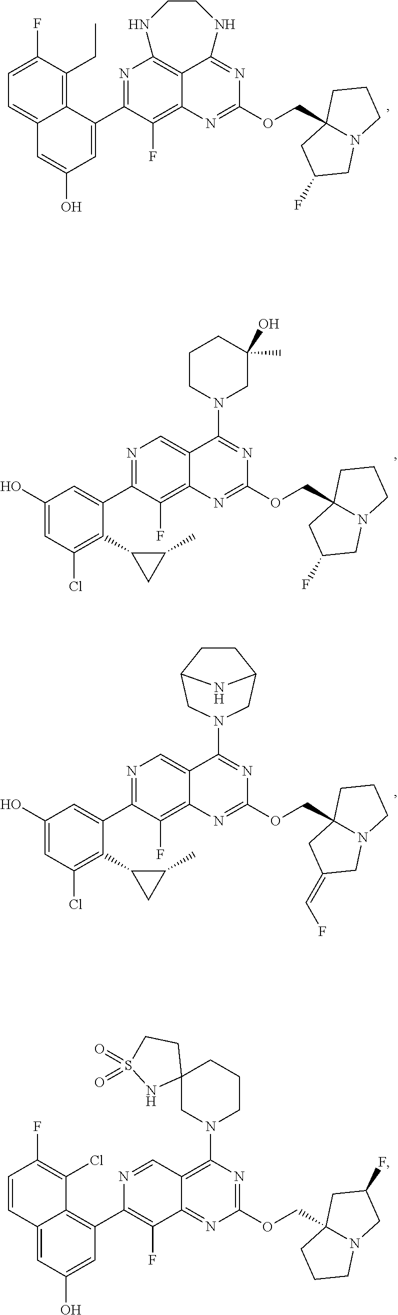 [SMILES: CCc1c(F)ccc2cc(O)cc(-c3nc4c5c(nc(OC[C@@]67CCCN6C[C@H](F)C7)nc5c3F)NCCN4)c12 ; C[C@@H]1C[C@@H]1c1c(Cl)cc(O)cc1-c1ncc2c(N3CCC[C@@](C)(O)C3)nc(OC[C@@]34CCCN3C[C@H](F)C4)nc2c1F ; C[C@@H]1C[C@@H]1c1c(Cl)cc(O)cc1-c1ncc2c(N3CC4CCC(C3)N4)nc(OC[C@@]34CCCN3C/C(=C\F)C4)nc2c1F ; O=S1(=O)CCC2(CCCN(c3nc(OC[C@@]45CCCN4C[C@H](F)C5)nc4c(F)c(-c5cc(O)cc6ccc(F)c(Cl)c56)ncc34)C2)N1] 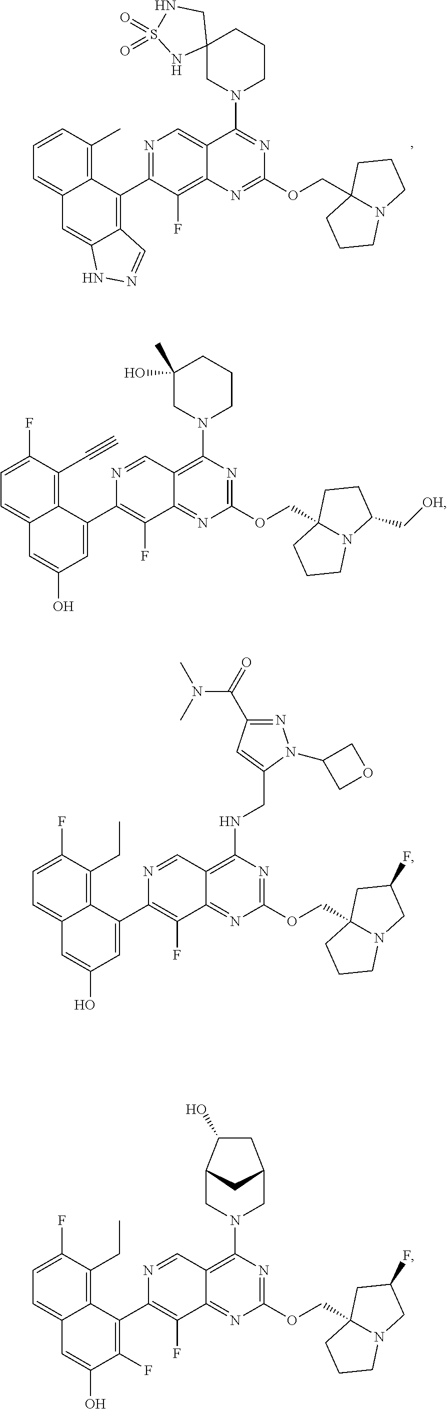 [SMILES: Cc1cccc2cc3[nH]ncc3c(-c3ncc4c(N5CCCC6(CNS(=O)(=O)N6)C5)nc(OCC56CCCN5CCC6)nc4c3F)c12 ; C#Cc1c(F)ccc2cc(O)cc(-c3ncc4c(N5CCC[C@@](C)(O)C5)nc(OC[C@@]56CCCN5[C@@H](CO)CC6)nc4c3F)c12 ; CCc1c(F)ccc2cc(O)cc(-c3ncc4c(NCc5cc(C(=O)N(C)C)nn5C5COC5)nc(OC[C@@]56CCCN5C[C@H](F)C6)nc4c3F)c12 ; CCc1c(F)ccc2cc(O)c(F)c(-c3ncc4c(N5C[C@@H]6C[C@H](C5)[C@H](O)C6)nc(OC[C@@]56CCCN5C[C@H](F)C6)nc4c3F)c12] 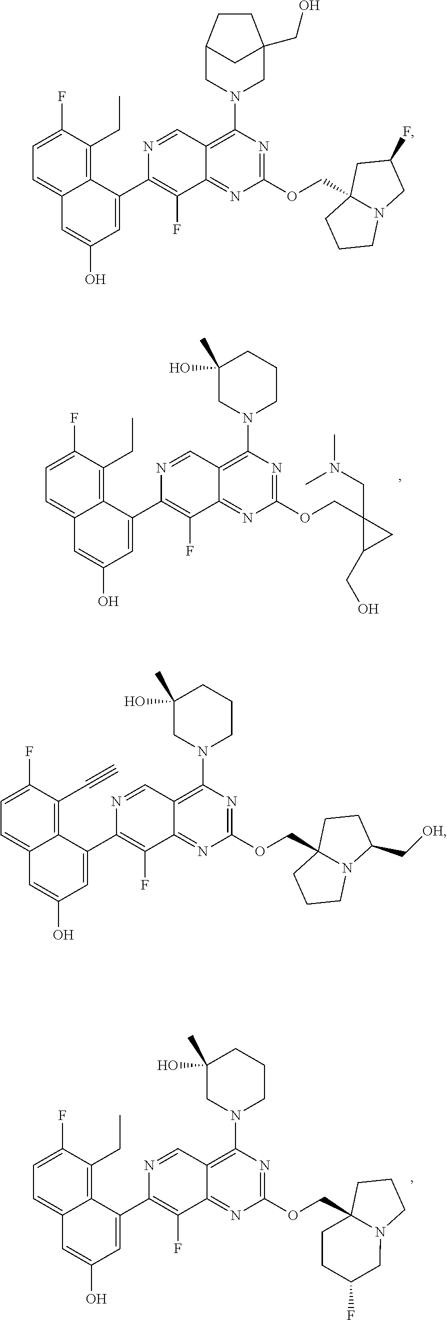 [SMILES: CCc1c(F)ccc2cc(O)cc(-c3ncc4c(N5CC6CCC(CO)(C6)C5)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OCC5(CN(C)C)CC5CO)nc4c3F)c12 ; C#Cc1c(F)ccc2cc(O)cc(-c3ncc4c(N5CCC[C@@](C)(O)C5)nc(OC[C@]56CCCN5[C@H](CO)CC6)nc4c3F)c12 ; CCc1c(F)ccc2cc(O)cc(-c3ncc4c(N5CCC[C@@](C)(O)C5)nc(OC[C@@]56CCCN5C[C@H](F)CC6)nc4c3F)c12] 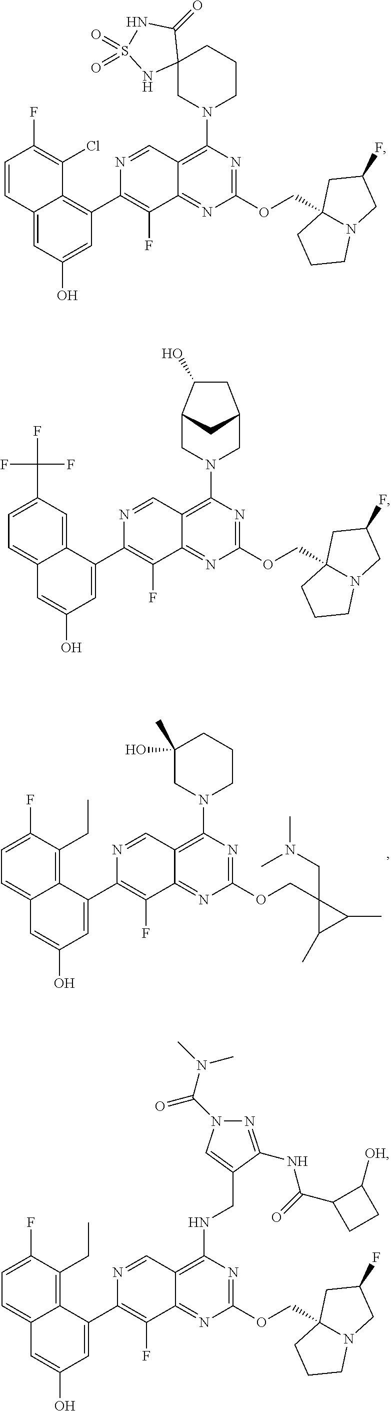 [SMILES: O=C1NS(=O)(=O)NC12CCCN(c1nc(OC[C@@]34CCCN3C[C@H](F)C4)nc3c(F)c(-c4cc(O)cc5ccc(F)c(Cl)c45)ncc13)C2 ; Oc1cc(-c2ncc3c(N4C[C@@H]5C[C@H](C4)[C@H](O)C5)nc(OC[C@@]45CCCN4C[C@H](F)C5)nc3c2F)c2cc(C(F)(F)F)ccc2c1 ; CCc1c(F)ccc2cc(O)cc(-c3ncc4c(N5CCC[C@@](C)(O)C5)nc(OCC5(CN(C)C)C(C)C5C)nc4c3F)c12 ; CCc1c(F)ccc2cc(O)cc(-c3ncc4c(NCc5cn(C(=O)N(C)C)nc5NC(=O)C5CCC5O)nc(OC[C@@]56CCCN5C[C@H](F)C6)nc4c3F)c12] 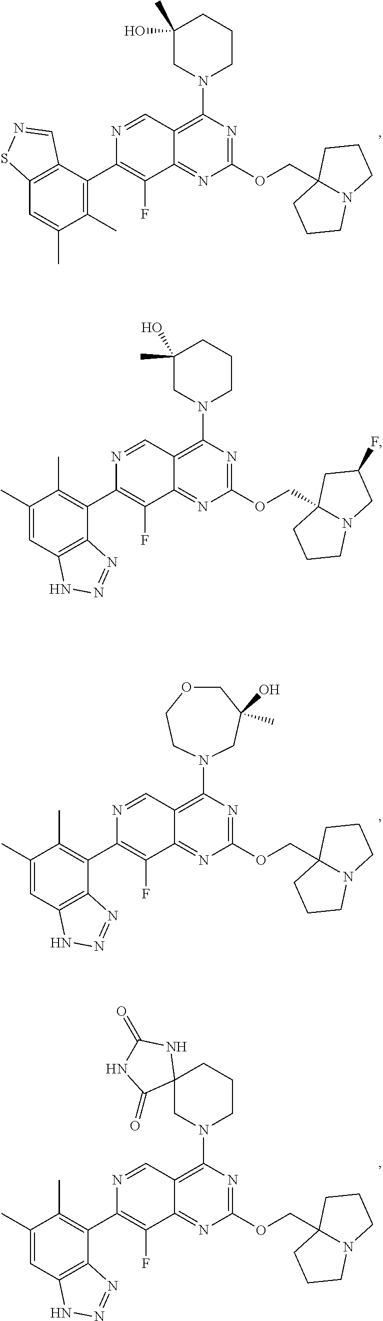 [SMILES: Cc1cc2sncc2c(-c2ncc3c(N4CCC[C@@](C)(O)C4)nc(OCC45CCCN4CCC5)nc3c2F)c1C ; Cc1cc2[nH]nnc2c(-c2ncc3c(N4CCC[C@@](C)(O)C4)nc(OC[C@@]45CCCN4C[C@H](F)C5)nc3c2F)c1C ; Cc1cc2[nH]nnc2c(-c2ncc3c(N4CCOC[C@@](C)(O)C4)nc(OCC45CCCN4CCC5)nc3c2F)c1C ; Cc1cc2[nH]nnc2c(-c2ncc3c(N4CCCC5(C4)NC(=O)NC5=O)nc(OCC45CCCN4CCC5)nc3c2F)c1C] 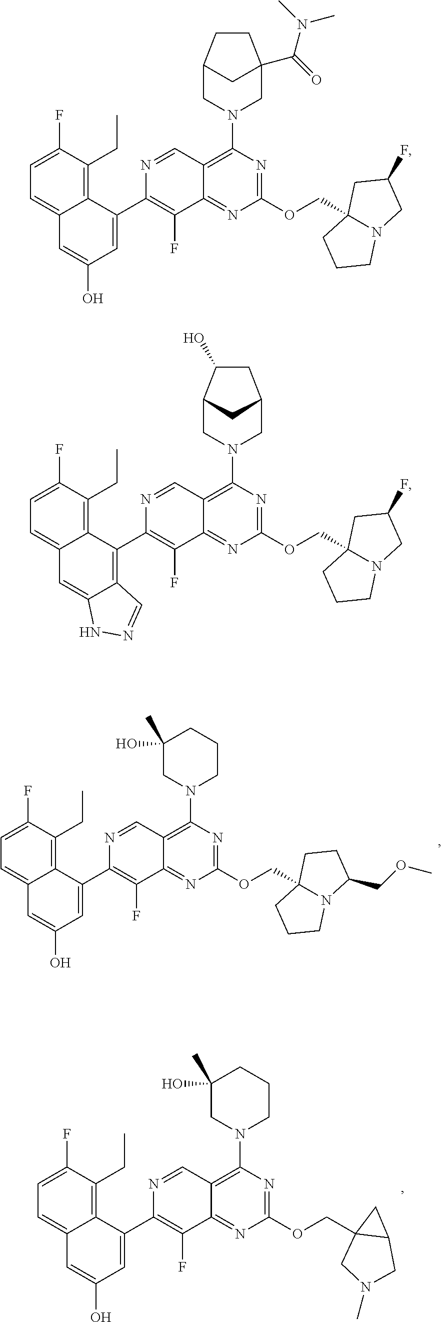 [SMILES: CCc1c(F)ccc2cc(O)cc(-c3ncc4c(N5CC6CCC(C(=O)N(C)C)(C6)C5)nc(OC[C@@]56CCCN5C[C@H](F)C6)nc4c3F)c12 ; CCc1c(F)ccc2cc3[nH]ncc3c(-c3ncc4c(N5C[C@@H]6C[C@H](C5)[C@H](O)C6)nc(OC[C@@]56CCCN5C[C@H](F)C6)nc4c3F)c12 ; CCc1c(F)ccc2cc(O)cc(-c3ncc4c(N5CCC[C@@](C)(O)C5)nc(OC[C@@]56CCCN5[C@H](COC)CC6)nc4c3F)c12 ; CCc1c(F)ccc2cc(O)cc(-c3ncc4c(N5CCC[C@@](C)(O)C5)nc(OCC56CC5CN(C)C6)nc4c3F)c12] 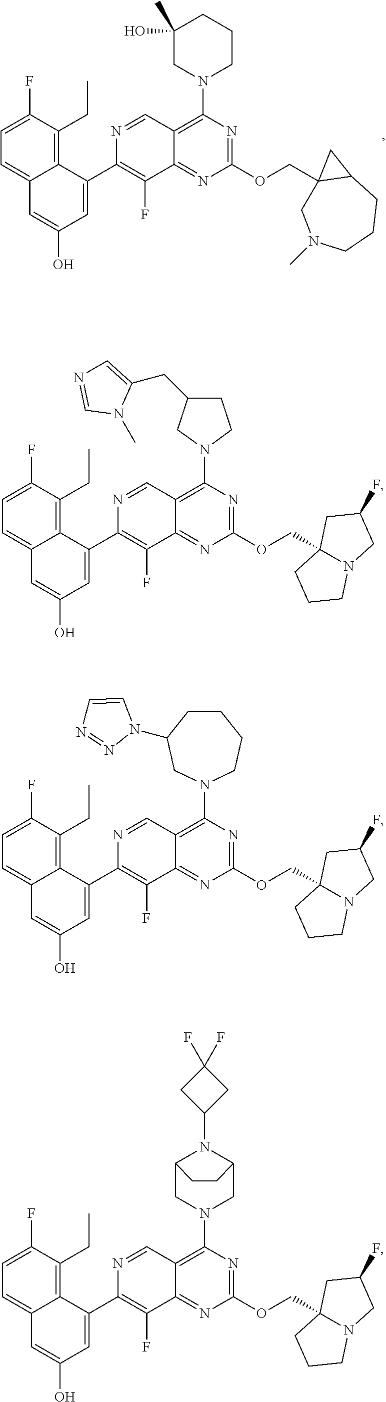 [SMILES: CCc1c(F)ccc2cc(O)cc(-c3ncc4c(N5CCC[C@@](C)(O)C5)nc(OCC56CC5CCCN(C)C6)nc4c3F)c12 ; CCc1c(F)ccc2cc(O)cc(-c3ncc4c(N5CCC(Cc6cncn6C)C5)nc(OC[C@@]56CCCN5C[C@H](F)C6)nc4c3F)c12 ; CCc1c(F)ccc2cc(O)cc(-c3ncc4c(N5CCCCC(n6ccnn6)C5)nc(OC[C@@]56CCCN5C[C@H](F)C6)nc4c3F)c12 ; CCc1c(F)ccc2cc(O)cc(-c3ncc4c(N5CC6CCC(C5)N6C5CC(F)(F)C5)nc(OC[C@@]56CCCN5C[C@H](F)C6)nc4c3F)c12] 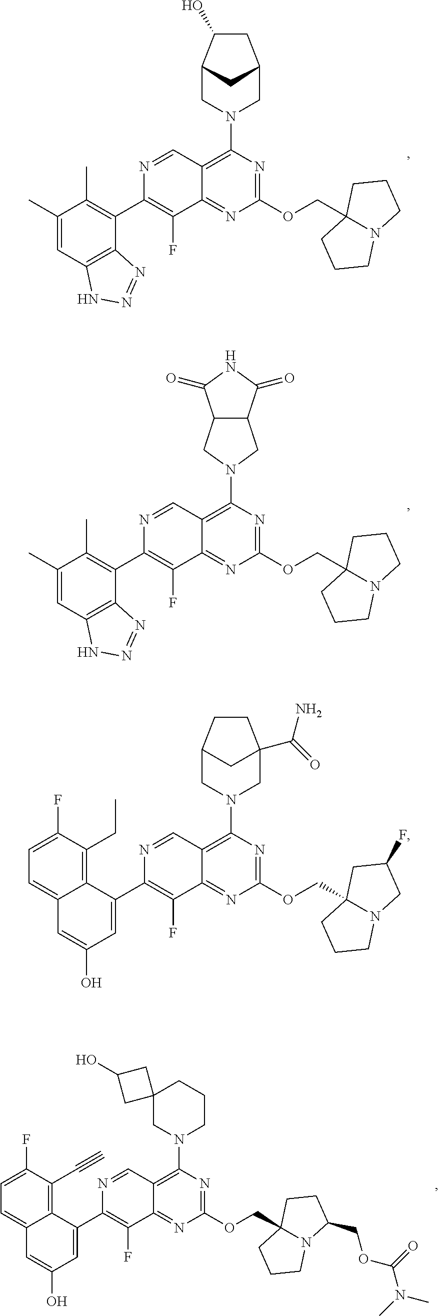 [SMILES: Cc1cc2[nH]nnc2c(-c2ncc3c(N4C[C@@H]5C[C@H](C4)[C@H](O)C5)nc(OCC45CCCN4CCC5)nc3c2F)c1C ; Cc1cc2[nH]nnc2c(-c2ncc3c(N4CC5C(=O)NC(=O)C5C4)nc(OCC45CCCN4CCC5)nc3c2F)c1C ; CCc1c(F)ccc2cc(O)cc(-c3ncc4c(N5CC6CCC(C(N)=O)(C6)C5)nc(OC[C@@]56CCCN5C[C@H](F)C6)nc4c3F)c12 ; C#Cc1c(F)ccc2cc(O)cc(-c3ncc4c(N5CCCC6(CC(O)C6)C5)nc(OC[C@]56CCCN5[C@H](COC(=O)N(C)C)CC6)nc4c3F)c12] 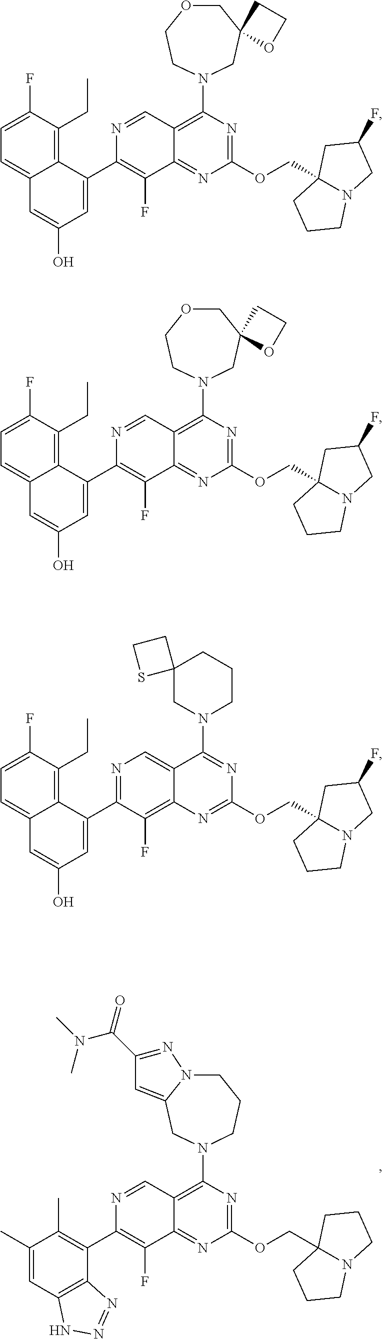 [SMILES: CCc1c(F)ccc2cc(O)cc(-c3ncc4c(N5CCOC[C@@]6(CCO6)C5)nc(OC[C@@]56CCCN5C[C@H](F)C6)nc4c3F)c12 ; CCc1c(F)ccc2cc(O)cc(-c3ncc4c(N5CCOC[C@]6(CCO6)C5)nc(OC[C@@]56CCCN5C[C@H](F)C6)nc4c3F)c12 ; CCc1c(F)ccc2cc(O)cc(-c3ncc4c(N5CCCC6(CCS6)C5)nc(OC[C@@]56CCCN5C[C@H](F)C6)nc4c3F)c12 ; Cc1cc2[nH]nnc2c(-c2ncc3c(N4CCCn5nc(C(=O)N(C)C)cc5C4)nc(OCC45CCCN4CCC5)nc3c2F)c1C] 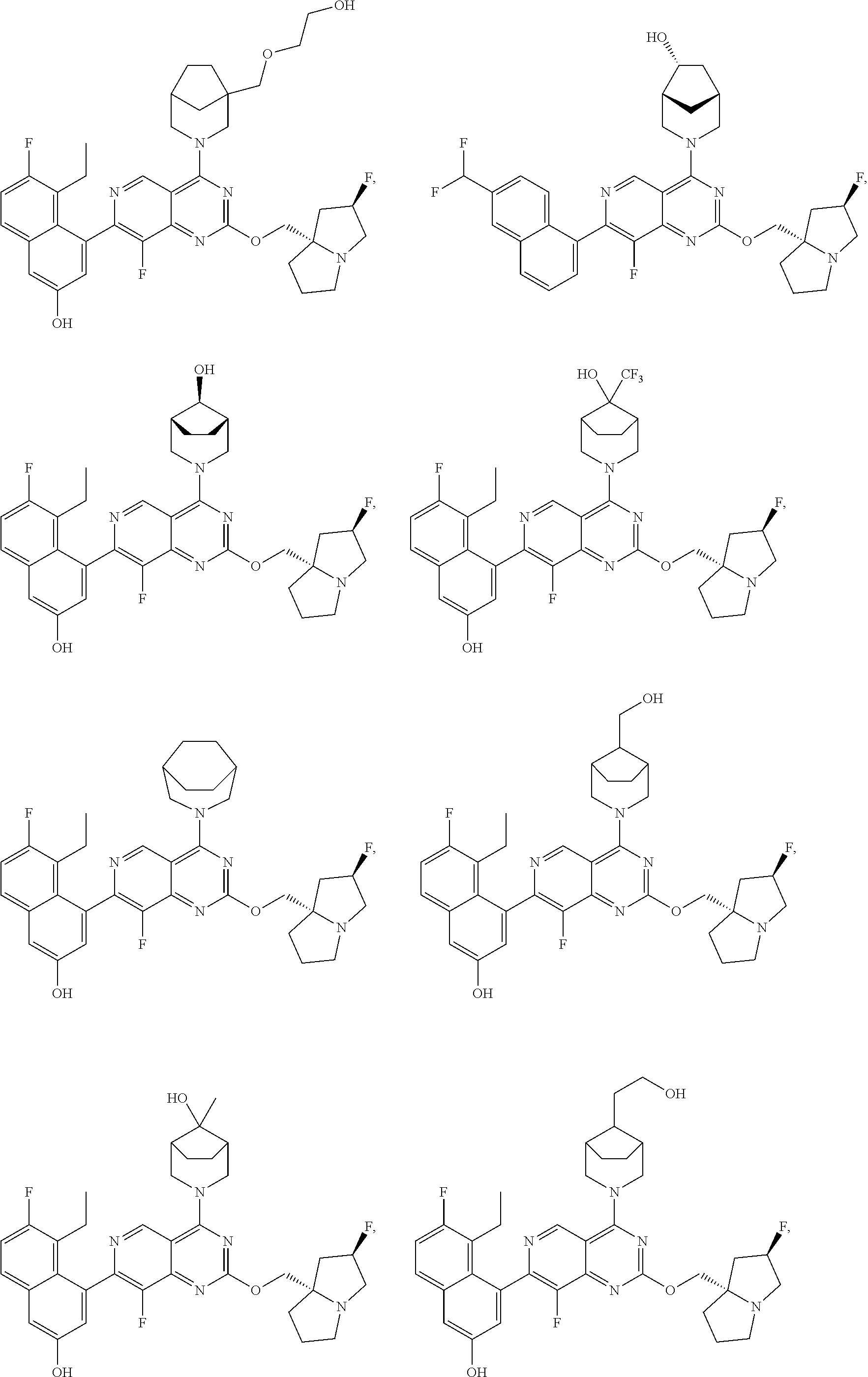 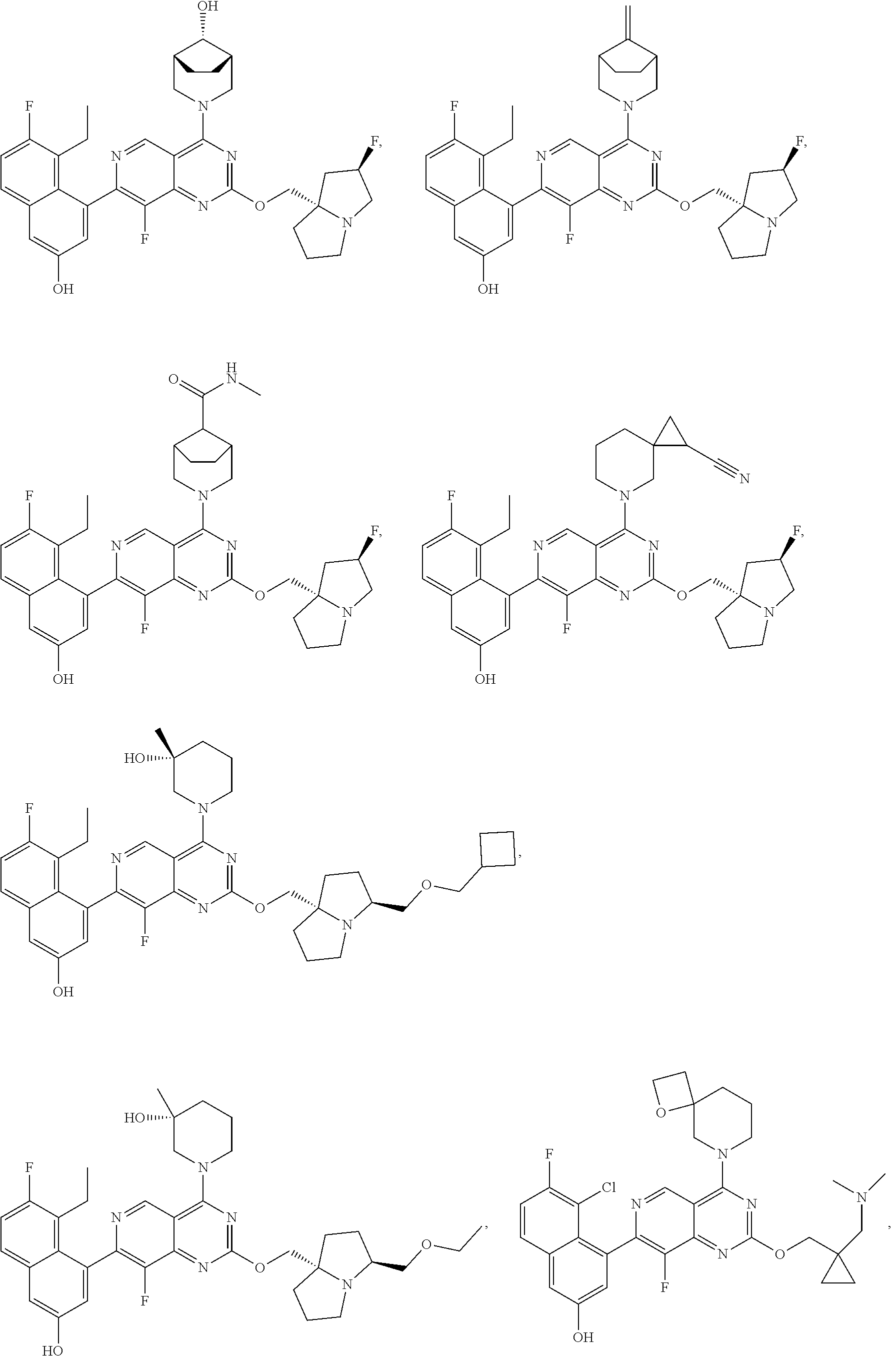 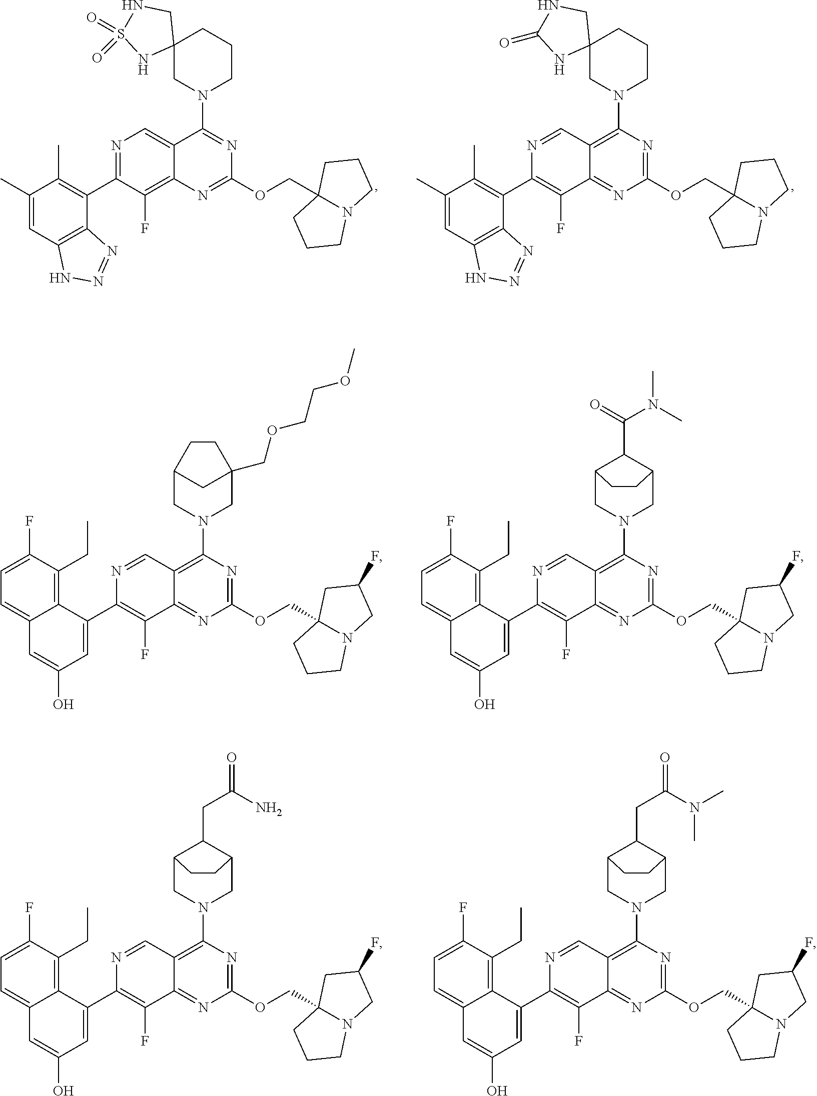 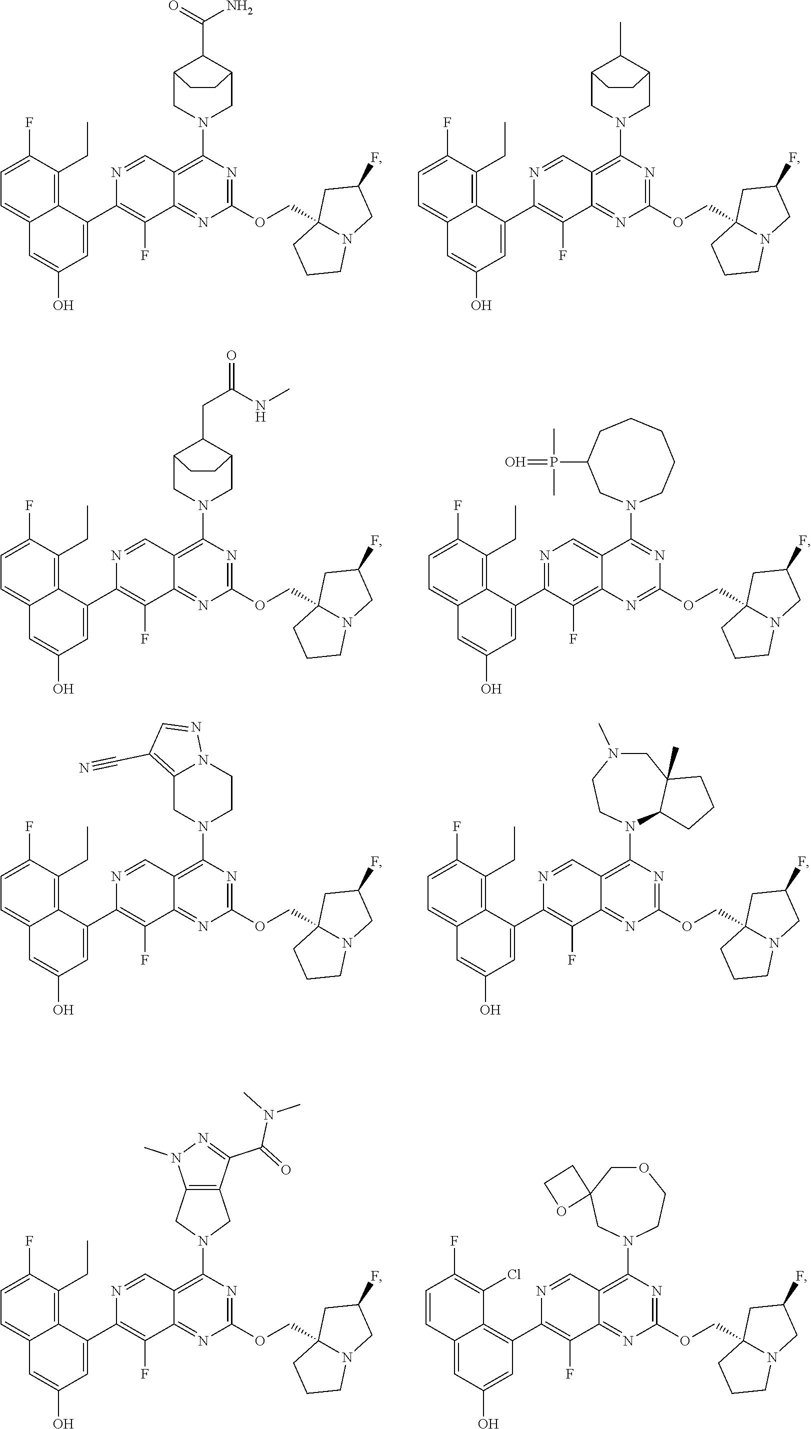 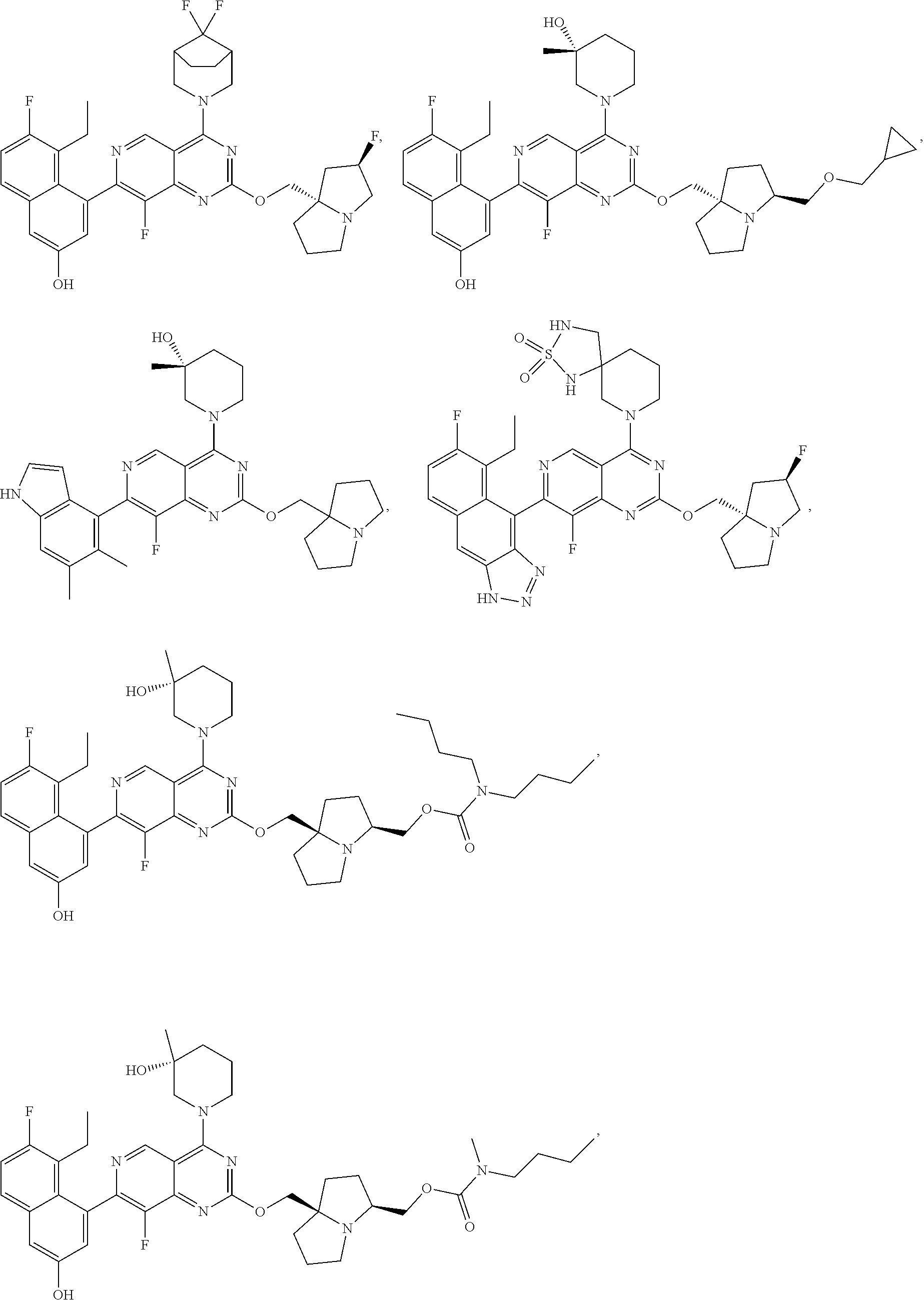 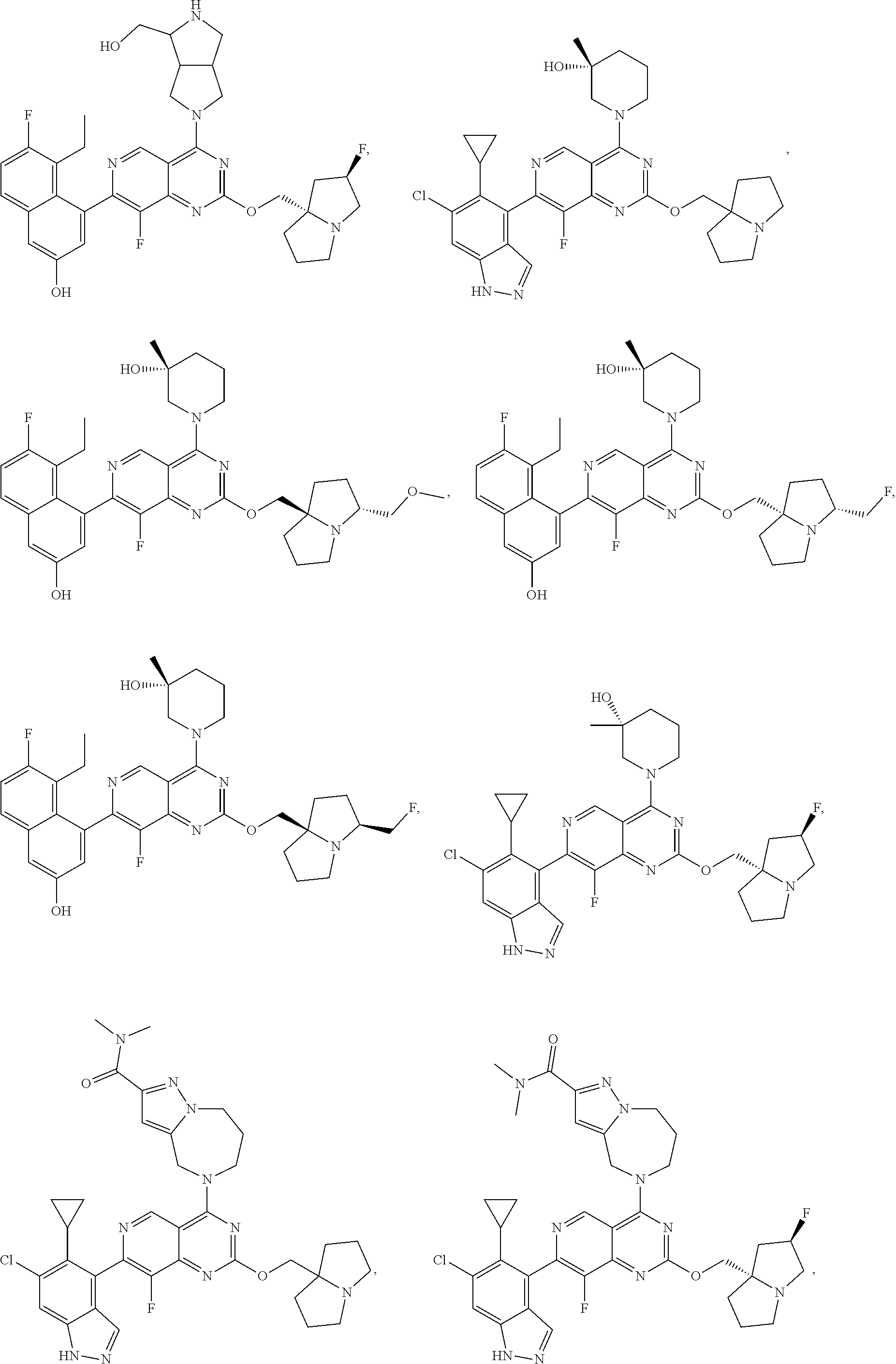 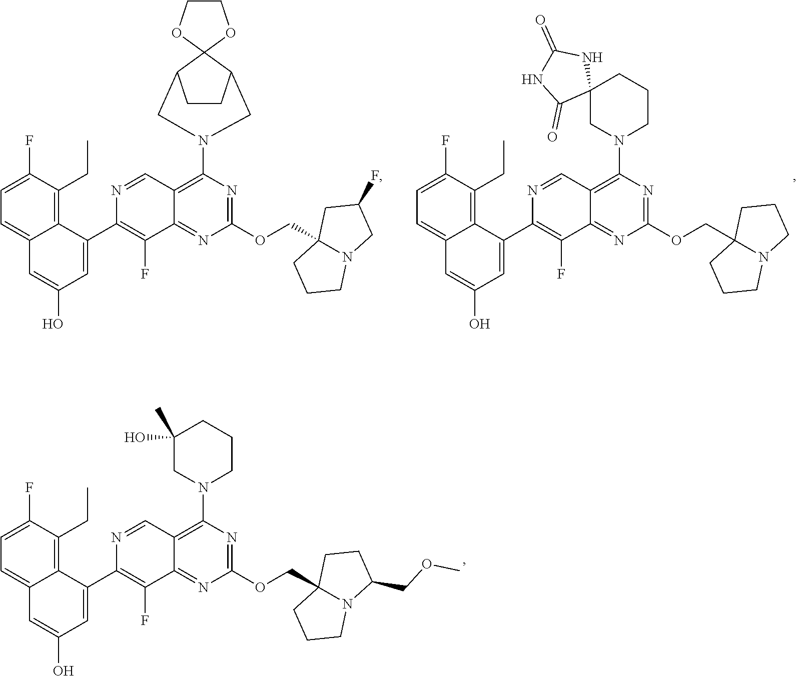 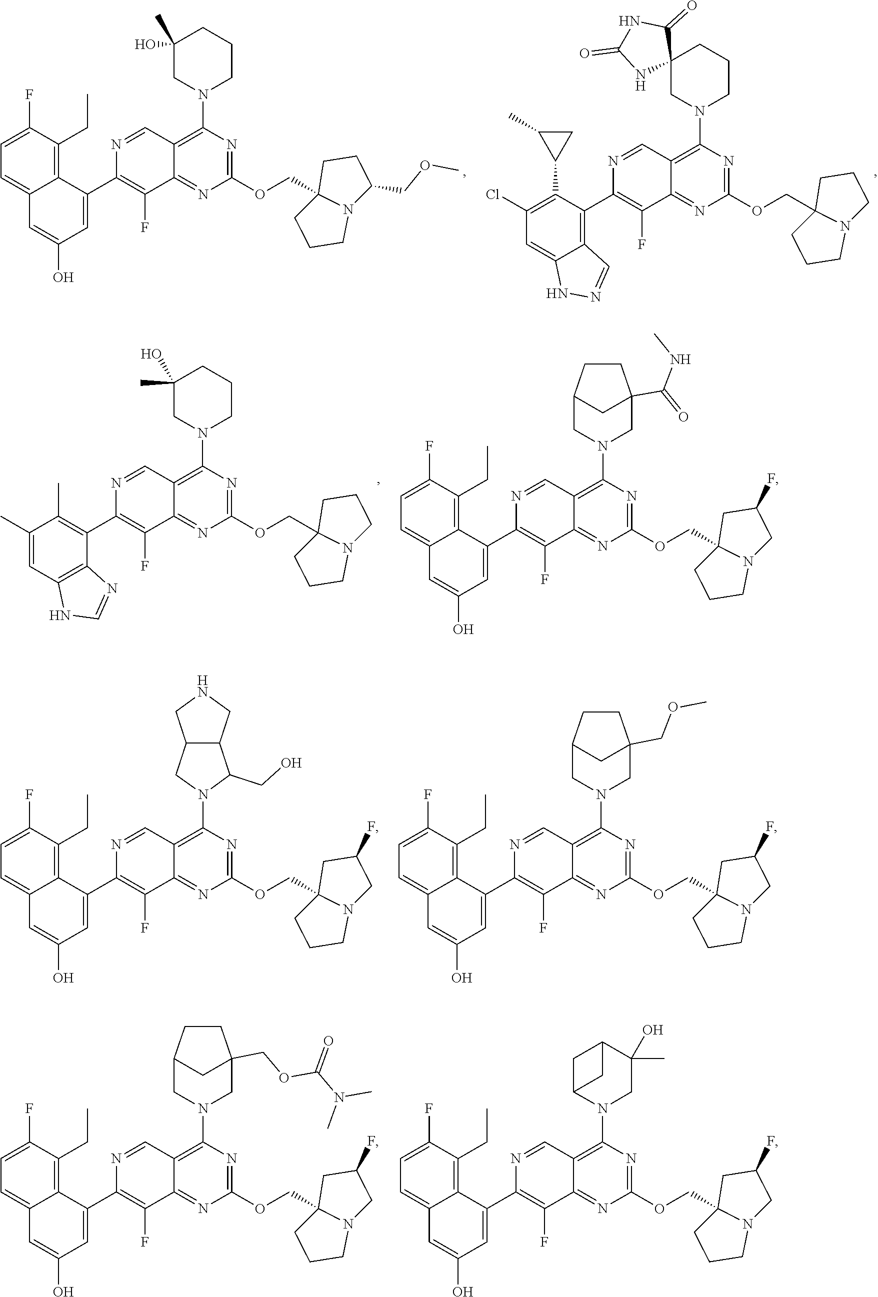 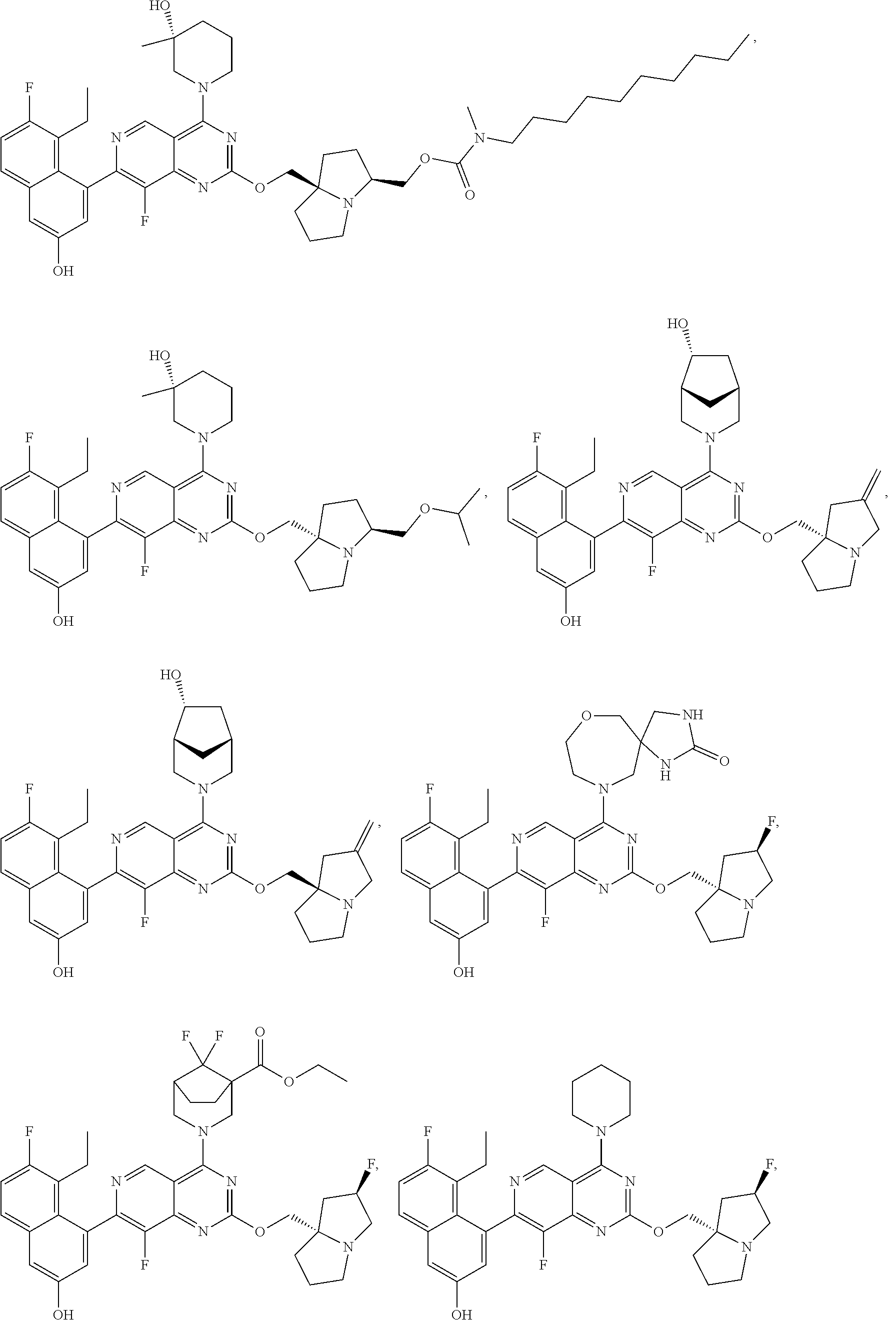 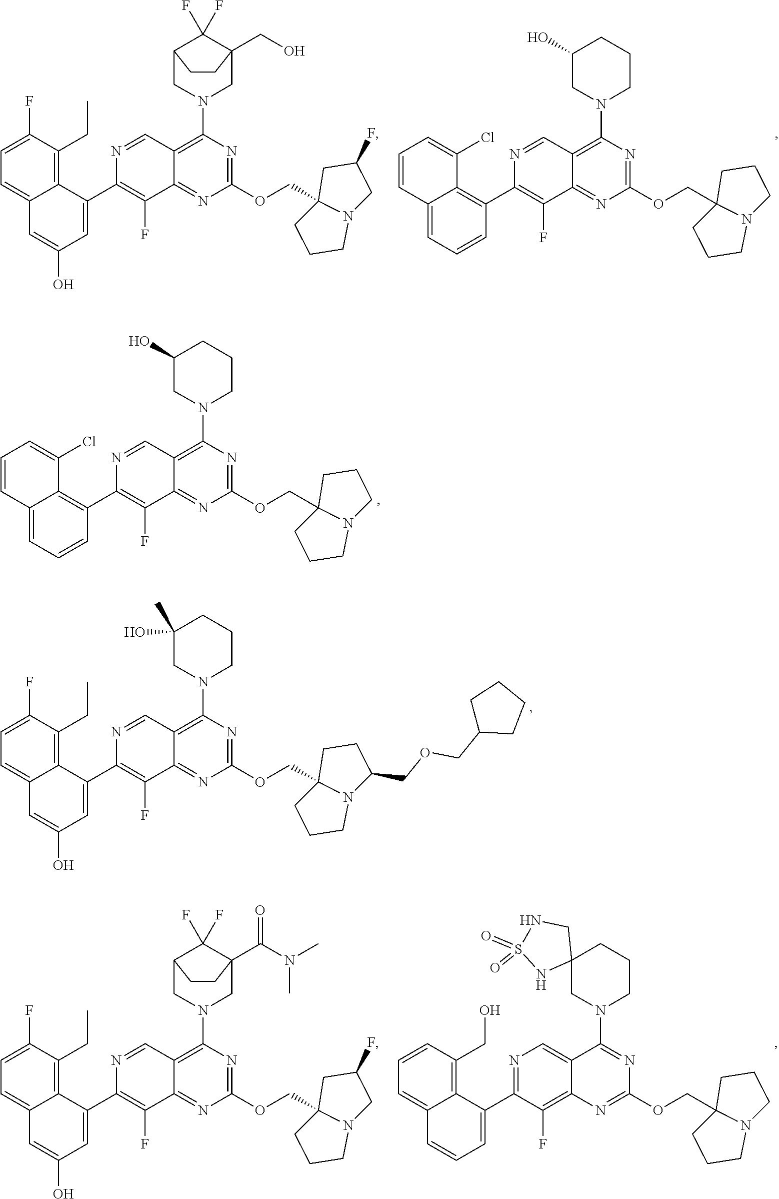 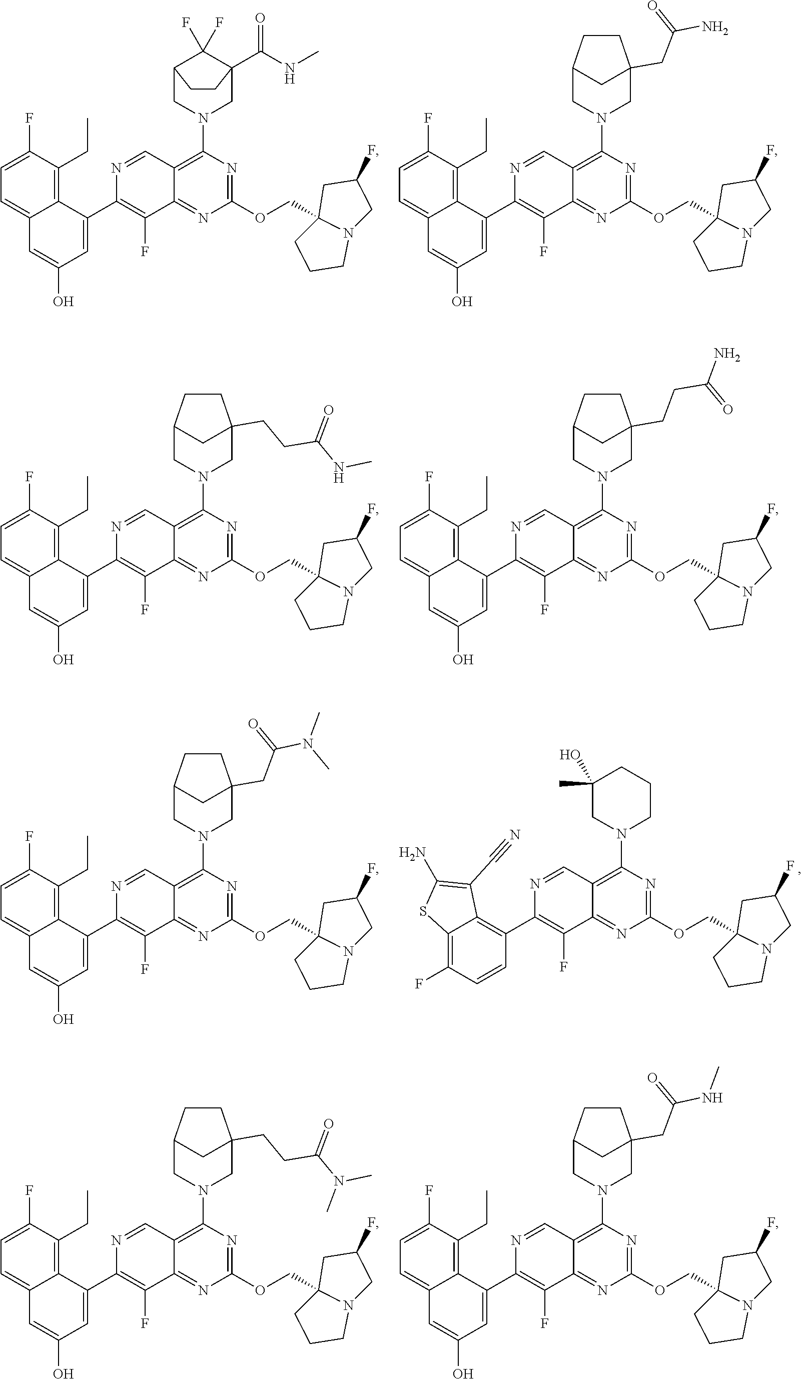 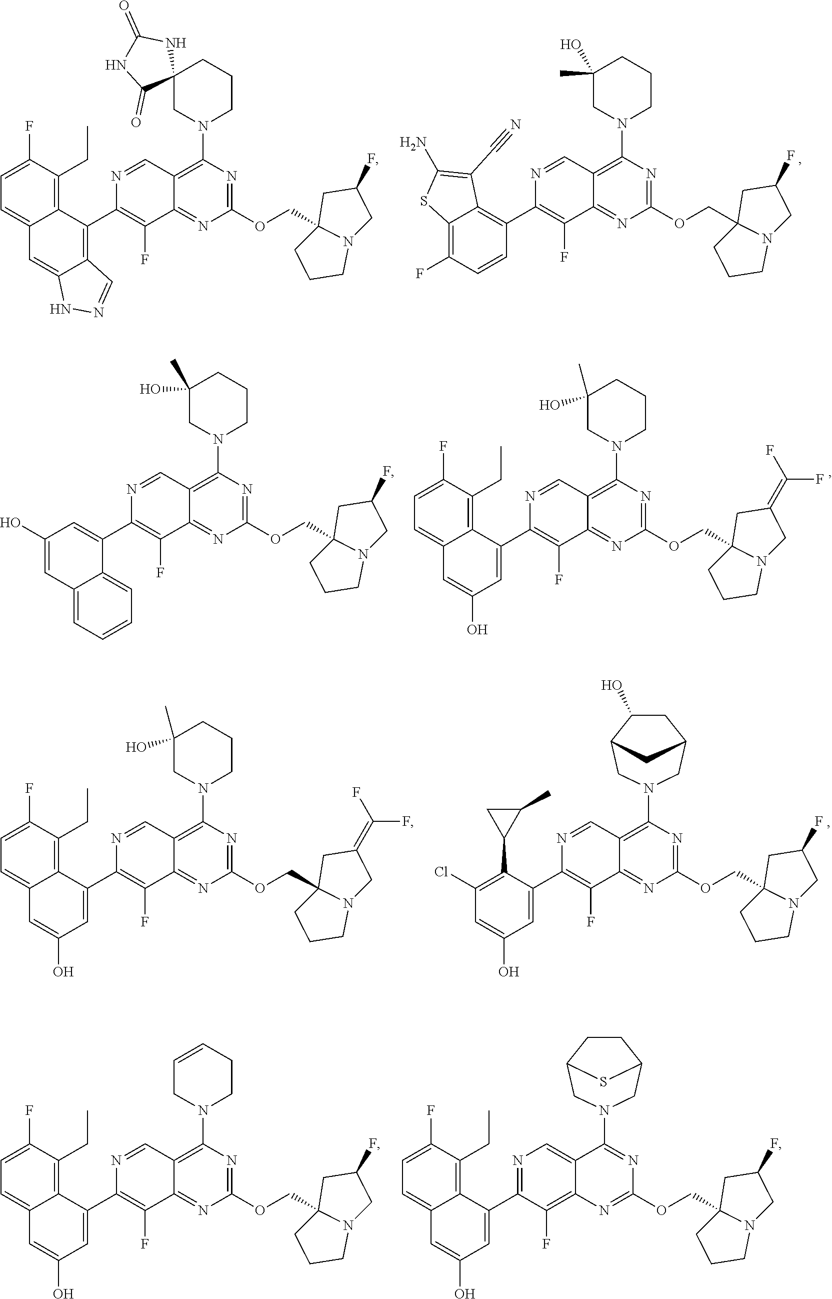 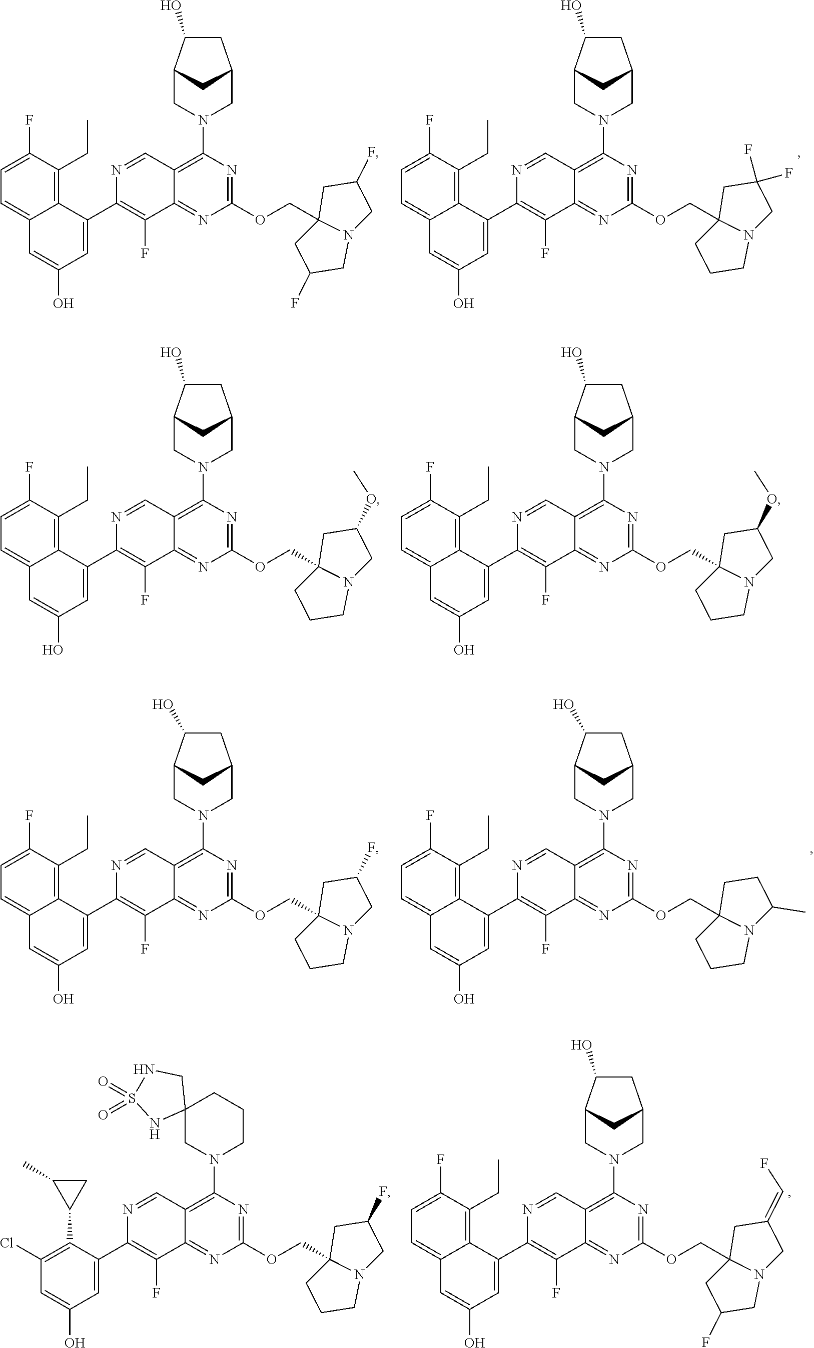 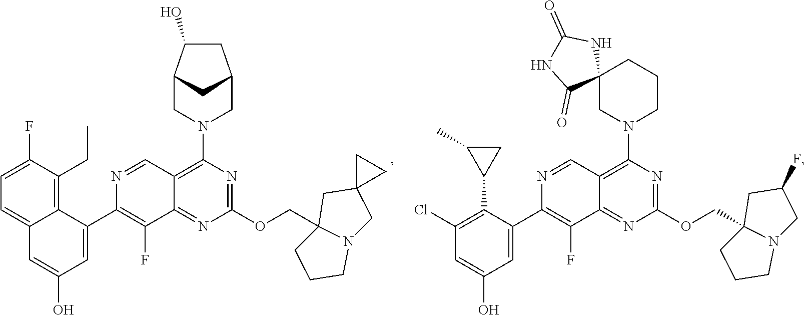 [SMILES: CCc1c(F)ccc2cc(O)cc(-c3ncc4c(N5C[C@@H]6C[C@H](C5)[C@H](O)C6)nc(OCC56CCCN5CC5(CC5)C6)nc4c3F)c12 ; C[C@@H]1C[C@@H]1c1c(Cl)cc(O)cc1-c1ncc2c(N3CCC[C@]4(C3)NC(=O)NC4=O)nc(OC[C@@]34CCCN3C[C@H](F)C4)nc2c1F] 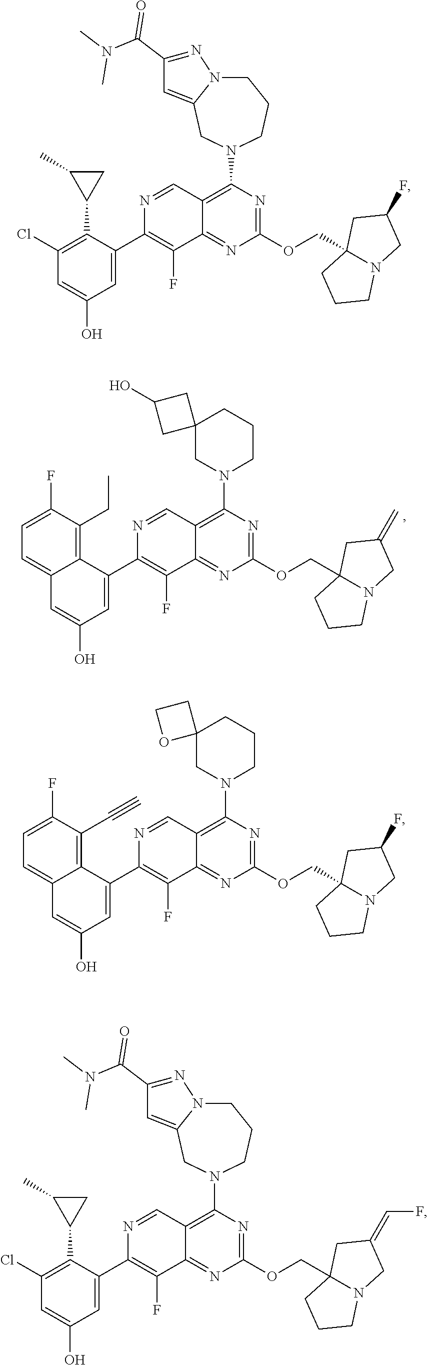 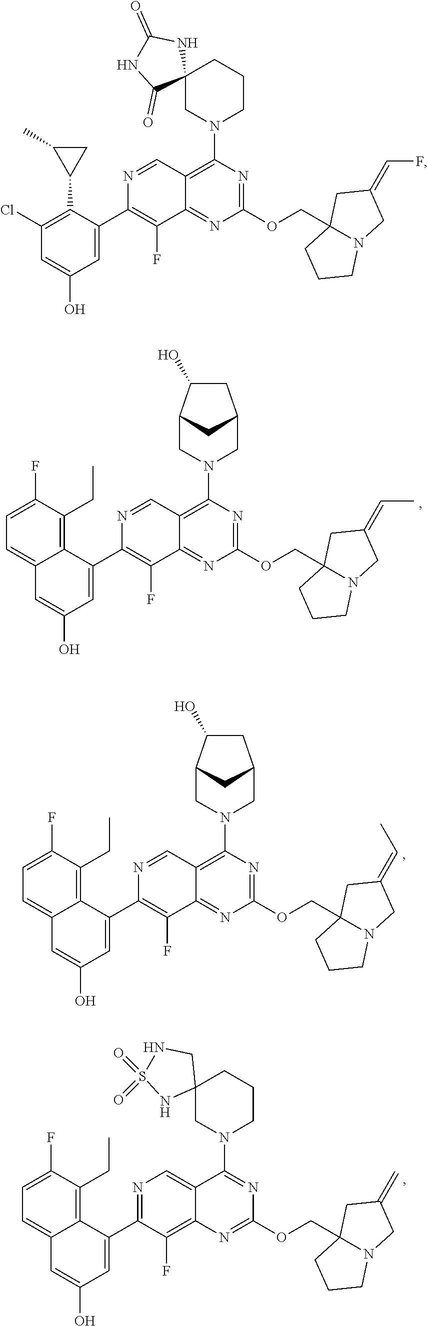 [SMILES: C[C@@H]1C[C@@H]1c1c(Cl)cc(O)cc1-c1ncc2c(N3CCC[C@]4(C3)NC(=O)NC4=O)nc(OCC34CCCN3C/C(=C\F)C4)nc2c1F ; C/C=C1\CN2CCCC2(COc2nc(N3C[C@@H]4C[C@H](C3)[C@H](O)C4)c3cnc(-c4cc(O)cc5ccc(F)c(CC)c45)c(F)c3n2)C1 ; C/C=C1/CN2CCCC2(COc2nc(N3C[C@@H]4C[C@H](C3)[C@H](O)C4)c3cnc(-c4cc(O)cc5ccc(F)c(CC)c45)c(F)c3n2)C1 ; C=C1CN2CCCC2(COc2nc(N3CCCC4(CNS(=O)(=O)N4)C3)c3cnc(-c4cc(O)cc5ccc(F)c(CC)c45)c(F)c3n2)C1] 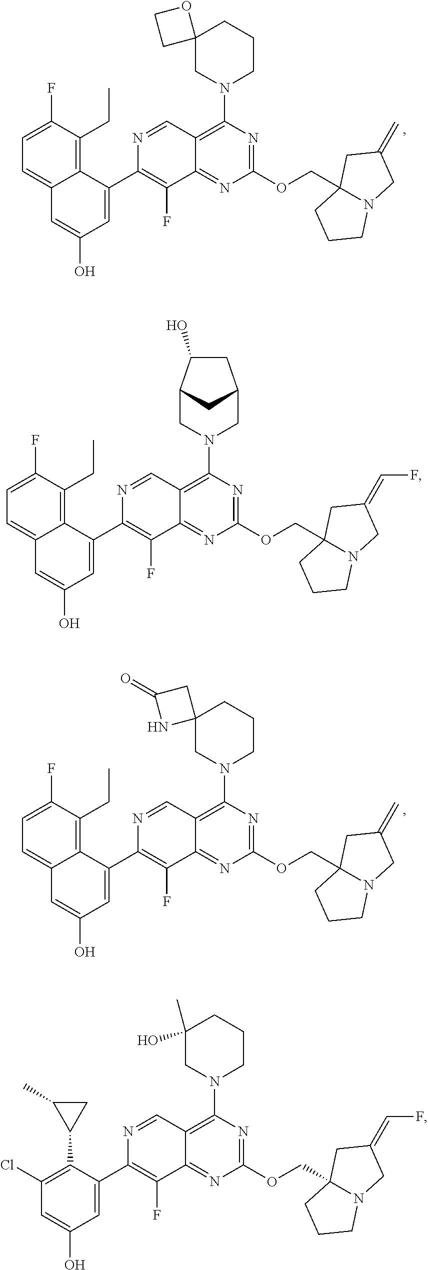 [SMILES: C=C1CN2CCCC2(COc2nc(N3CCCC4(CCO4)C3)c3cnc(-c4cc(O)cc5ccc(F)c(CC)c45)c(F)c3n2)C1 ; CCc1c(F)ccc2cc(O)cc(-c3ncc4c(N5C[C@@H]6C[C@H](C5)[C@H](O)C6)nc(OCC56CCCN5C/C(=C\F)C6)nc4c3F)c12 ; C=C1CN2CCCC2(COc2nc(N3CCCC4(CC(=O)N4)C3)c3cnc(-c4cc(O)cc5ccc(F)c(CC)c45)c(F)c3n2)C1 ; C[C@@H]1C[C@@H]1c1c(Cl)cc(O)cc1-c1ncc2c(N3CCC[C@@](C)(O)C3)nc(OC[C@@]34CCCN3C/C(=C\F)C4)nc2c1F] 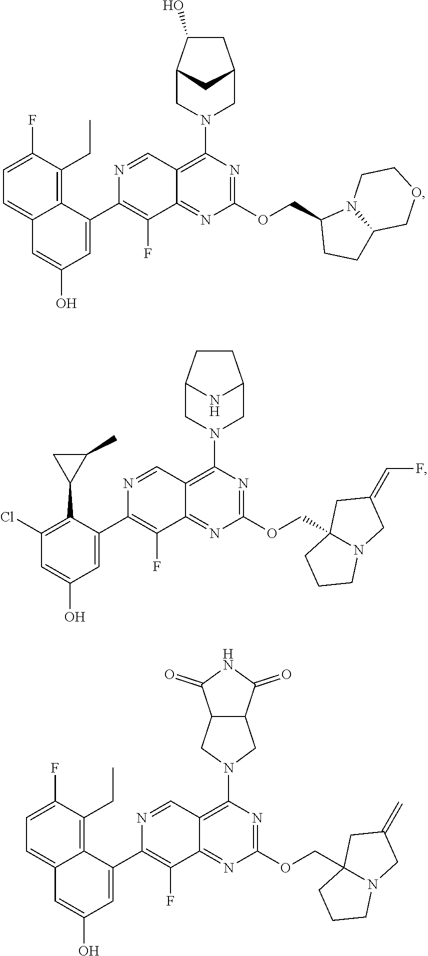 [SMILES: CCc1c(F)ccc2cc(O)cc(-c3ncc4c(N5C[C@@H]6C[C@H](C5)[C@H](O)C6)nc(OC[C@@H]5CC[C@H]6COCCN65)nc4c3F)c12 ; C[C@@H]1C[C@@H]1c1c(Cl)cc(O)cc1-c1ncc2c(N3CC4CCC(C3)N4)nc(OC[C@@]34CCCN3C/C(=C\F)C4)nc2c1F ; C=C1CN2CCCC2(COc2nc(N3CC4C(=O)NC(=O)C4C3)c3cnc(-c4cc(O)cc5ccc(F)c(CC)c45)c(F)c3n2)C1]
and pharmaceutically acceptable salts thereof.

60. A pharmaceutical composition, comprising a therapeutically effective amount of a compound of any of claims 1-59 or a pharmaceutically acceptable salt thereof, and a pharmaceutically acceptable excipient.

61. A method for inhibiting the wild type KRas, KRas G12A, KRas G12C, KRas G12D, KRas G12R, KRas G12S, KRas G12V, KRas G13D or KRas Q61H activity in a cell, comprising contacting the cell in which inhibition of KRas activity is desired with an effective amount of a compound of according to any of claims 1-59 or a pharmaceutically acceptable salt thereof, or the pharmaceutical composition of claim 60.

62. A method for treating cancer comprising administering to a patient having cancer a therapeutically effective amount of a compound according to any of claims 1-59 or a pharmaceutically acceptable salt thereof, or the pharmaceutical composition of claim 60.

63. The method of claim 62, wherein the therapeutically effective amount of the compound is between about 0.01 to 100 mg/kg per day.

64. The method of claim 63, wherein the therapeutically effective amount of the compound is between about 0.1 to 50 mg/kg per day.

65. The method of claim 62, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma): Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

66. The method of claim 65, wherein the cancer is a KRas G12A-associated cancer.

67. The method of claim 65, wherein the cancer is a KRas G12C-associated cancer.

68. The method of claim 65, wherein the cancer is a KRas G12D-associated cancer.

69. The method of claim 65, wherein the cancer is a KRas G12R-associated cancer.

70. The method of claim 65, wherein the cancer is a KRas G12S-associated cancer.

71. The method of claim 65, wherein the cancer is a KRas G12V-associated cancer.

72. The method of claim 65, wherein the cancer is a KRas G13D-associated cancer.

73. The method of claim 65, wherein the cancer is a KRas Q61H-associated cancer.

74. The method of claim 65, wherein the cancer is a KRas G12A-associated cancer.

75. The method of claim 65, wherein the cancer is associated with at least one of wild type KRas, KRas G12A, KRas G12C, KRas G12D, KRas G12R, KRas G12S, KRas G12V, KRas G13D or KRas Q61H.

76. The method of any of claims 65-75, wherein the cancer is non-small cell lung cancer, small cell lung cancer, colorectal cancer, rectal cancer or pancreatic cancer.

77. A method for treating cancer in a patient in need thereof, the method comprising (a) determining that the cancer is associated with wild type KRas or a KRas G12A, KRas G12C, KRas G12D, KRas G12R, KRas G12S, KRas G12V, KRas G13D or KRas Q61H mutation; and (b) administering to the patient a therapeutically effective amount of a compound according to any of claims 1-59 or a pharmaceutically acceptable salt thereof, or the pharmaceutical composition of claim 60.

78. The method of any one of claims 65-77, wherein the administering is done via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

79. The method of claim 78, wherein the administration route is oral.

80. The method of claim 78, wherein the administration is intravenous injection.

81. The method of claim 78, wherein the administration route is intramuscular injection.

82. The method of claim 78, wherein the administration route utilizes a delivery device.

83. The method of claim 78, wherein administration is done in a hospital setting.
